(19) United States
(12) Patent Application Publication
(10) Pub. No.: US 2024/0147851 A1
(43) Pub. Date: May 2, 2024

(54) LIGHT EMITTING DEVICE AND NITROGEN-CONTAINING COMPOUND FOR LIGHT EMITTING DEVICE

(71) Applicant: Samsung Display Co., Ltd., Yongin-si (KR)

(72) Inventors: YESEUL LEE, Yongin-si (KR); HYEONGMIN KIM, Yongin-si (KR); HEECHOON AHN, Yongin-si (KR); HYUNAH UM, Yongin-si (KR); Juhui YUN, Yongin-si (KR); MORAN HA, Yongin-si (KR)

(21) Appl. No.: 18/355,313

(22) Filed: Jul. 19, 2023

(30) Foreign Application Priority Data
Sep. 19, 2022 (KR) 10-2022-0117715
Jan. 27, 2023 (KR) 10-2023-0010668

(51) Int. Cl. H10K 85/60; C07D 209/86; C07D 405/14; C07F 7/08; C09K 11/02; C09K 11/06; H10K 85/30; H10K 85/40

(52) U.S. Cl. CPC H10K 85/6572; C07D 209/86; C07D 405/14; C07F 7/0812; C09K 11/02; C09K 11/06; H10K 85/346; H10K 85/40; H10K 85/6574; H10K 50/12

(57) ABSTRACT

A light emitting device of one or more embodiments includes a first electrode, a second electrode oppositely provided to the first electrode, and an emission layer provided between the first electrode and the second electrode, wherein the emission layer includes a first compound represented by Formula 1 below.

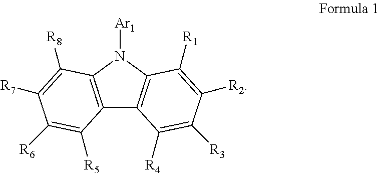

### CROSS-REFERENCE TO RELATED APPLICATIONS

This application claims priority to and the benefit of Korean Patent Application No. 10-2022-0117715, filed on Sep. 19, 2022, and Korean Patent Application No. 10-2023-0010668, filed on Jan. 27, 2023, the entire content of each of which is hereby incorporated by reference.

### BACKGROUND

### 1. Field

One or more aspects of embodiments of the present disclosure herein relate to a light emitting device and a nitrogen-containing compound utilized in the light emitting device.

### 2. Description of the Related Art

Recently, the development of an organic electroluminescence display as an image display has been actively conducted. The organic electroluminescence display is different from a liquid crystal display and is a display of a self-luminescent type or kind in which holes and electrons injected from a first electrode and a second electrode recombine in an emission layer so that a light emitting material in the emission layer emits light to achieve display of images.

In the application of an organic electroluminescence device to a display, the decrease of a driving voltage and the increase of the emission efficiency and lifetime of the organic electroluminescence device are required or desired, and development of materials for an organic electroluminescence device, capable of suitably achieving these characteristics is being continuously required or desired.

### SUMMARY

One or more aspects of embodiments of the present disclosure are directed toward a light emitting device having improved emission efficiency and device life.

One or more aspects of embodiments of the present disclosure are also directed toward a nitrogen-containing compound which may improve the emission efficiency and device life of a light emitting device.

One or more embodiments of the present disclosure provide a light emitting device including a first electrode, a second electrode oppositely provided to the first electrode, and an emission layer provided between the first electrode and the second electrode, wherein the emission layer includes a first compound represented by Formula 1.

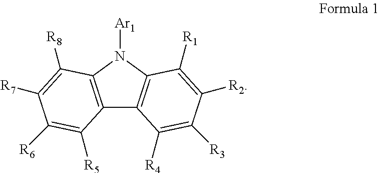

In Formula 1, Ar₁ may be a substituted or unsubstituted aryl group of 6 to 30 ring-forming carbon atoms, or a substituted or unsubstituted heteroaryl group of 2 to 30 ring-forming carbon atoms, R₁ to R₈ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and any one among R₅ to R₈ may be a substituent represented by Formula 2.

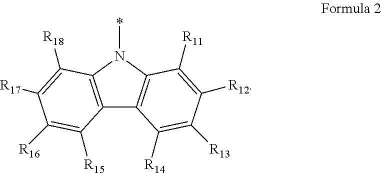

In Formula 2, R₁₁ to R₁₈ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and in Formula 1 and Formula 2, at least one of R₄ or R₁₄ may be a substituted or unsubstituted phenyl group or a substituent represented by Formula 3.

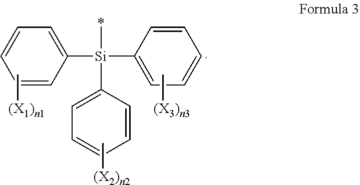

In Formula 3, X₁ to X₃ may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and n1 to n3 may each independently be an integer of 0 to 5.

In one or more embodiments, the first compound represented by Formula 1 may be represented by Formula 1-1.

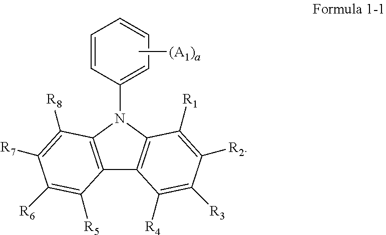

In Formula 1-1, A₁ is a hydrogen atom, a deuterium atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl, a substituted or unsubstituted biphenyl group, or combined with an adjacent group to form a ring, “a” is an integer of 0 to 5, and R₁ to R₈ may each independently be the same as defined in Formula 1.

In Formula 1-1, A₁ may be a substituent represented by any one among Formula 4-1 to Formula 4-11.

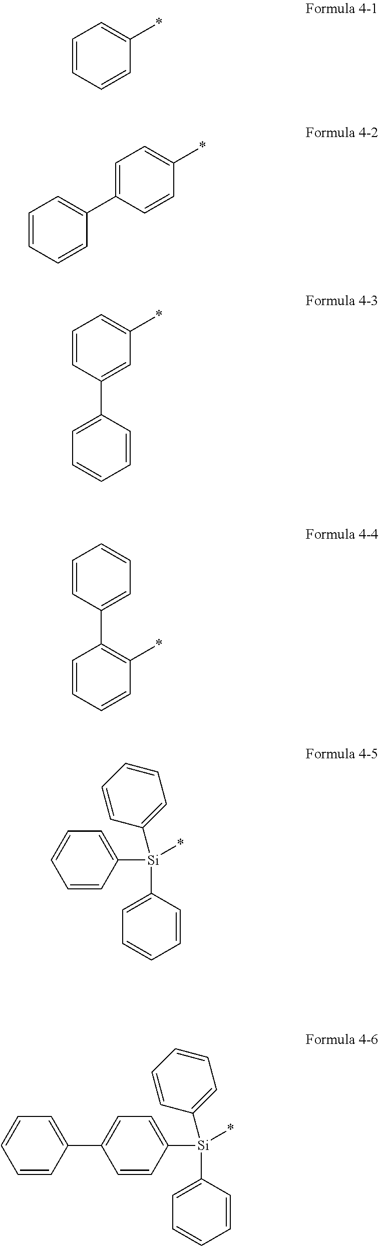 [SMILES: *c1ccccc1 ; *c1ccc(-c2ccccc2)cc1 ; *c1cccc(-c2ccccc2)c1 ; *c1ccccc1-c1ccccc1 ; *[Si](c1ccccc1)(c1ccccc1)c1ccccc1 ; *[Si](c1ccccc1)(c1ccccc1)c1ccc(-c2ccccc2)cc1]

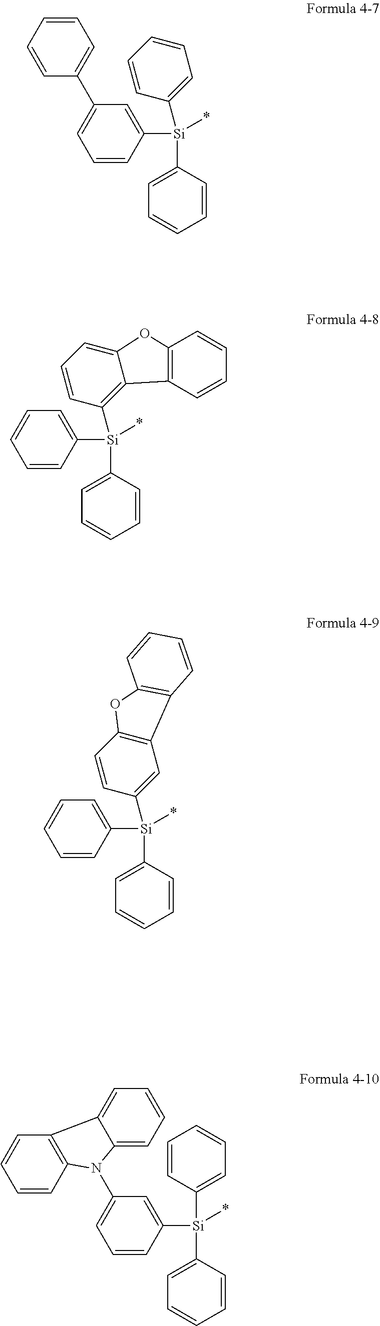 [SMILES: *[Si](c1ccccc1)(c1ccccc1)c1cccc(-c2ccccc2)c1 ; *[Si](c1ccccc1)(c1ccccc1)c1cccc2oc3ccccc3c12 ; *[Si](c1ccccc1)(c1ccccc1)c1ccc2oc3ccccc3c2c1 ; *[Si](c1ccccc1)(c1ccccc1)c1cccc(-n2c3ccccc3c3ccccc32)c1]

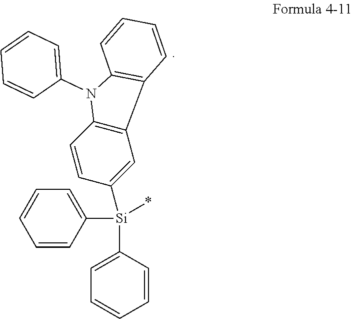 [SMILES: *[Si](c1ccccc1)(c1ccccc1)c1ccc2c(c1)c1ccccc1n2-c1ccccc1]

In Formula 4-1 to Formula 4-11, “ [symbol] ” is a position connected with Formula 1-1.

In one or more embodiments, the first compound represented by Formula 1 may be represented by Formula 1-2.

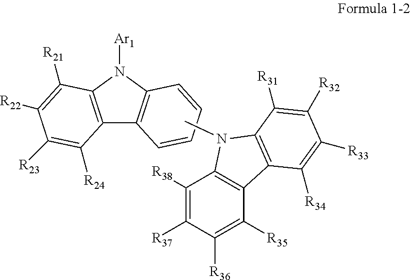

In Formula 1-2, R₂₁ to R₃₈ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, at least one of R₂₄ or R₃₄ is a substituted or unsubstituted phenyl group, or a substituent represented by Formula 3, and Ar₁ is the same as defined in Formula 1.

In one or more embodiments, the substituent represented by Formula 3 may be represented by Formula 3-1.

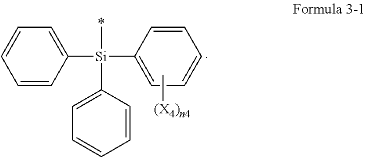

In Formula 3-1, X₄ may be a hydrogen atom, a deuterium atom, a cyano group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl group, a substituted or unsubstituted biphenyl group, or a substituted or unsubstituted heteroaryl group of 2 to 10 ring-forming carbon atoms, and/or combined with an adjacent group to form a ring, and n4 is an integer of 0 to 5.

In one or more embodiments, the first compound represented by Formula 1 may be represented by Formula 1-3 or Formula 1-4.

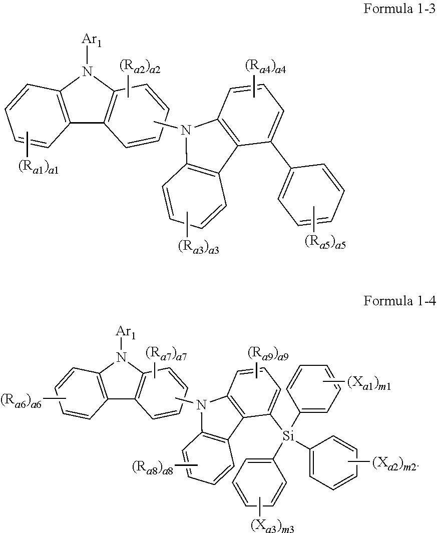

In Formula 1-3 and Formula 1-4, Rₐ₁ to Rₐ₉ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, Xₐ₁ to Xₐ₃ may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, a1, a3, a6 and a8 may each independently be an integer of 0 to 4, a2, a4, a7 and a9 may each independently be an integer of 0 to 3, a5 and m1 to m3 may each independently be an integer of 0 to 5, and Ar is the same as defined in Formula 1. Rₐ₅ and Xₐ₁ to Xₐ₃ may each independently be a hydrogen atom, a deuterium atom or a substituted or unsubstituted phenyl group.

In one or more embodiments, the first compound represented by Formula 1 may be represented by Formula 1-5 or Formula 1-6.

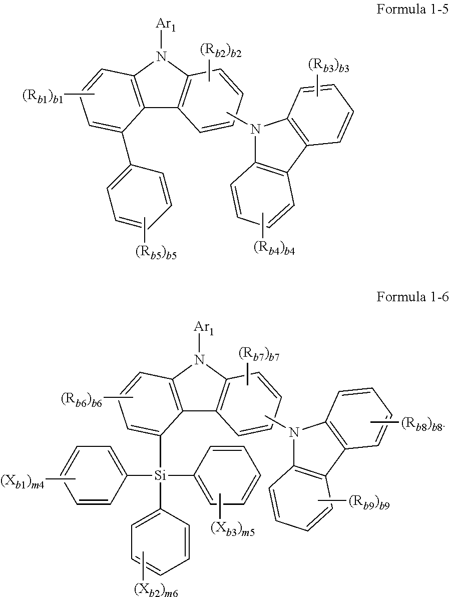

In Formula 1-5 and Formula 1-6, R_{b1} to R_{b9} may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, X_{b1} to X_{b3} may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, b1, b2, b6 and b7 may each independently be an integer of 0 to 3, b3, b4, b8 and b9 may each independently be an integer of 0 to 4, b5 and m4 to m6 may each independently be an integer of 0 to 5, and Ar₁ may be the same as defined in Formula 1. R_{b3}, R_{b4}, R_{b8} and R_{b9} may each independently be a hydrogen atom, a deuterium atom, a substituted or unsubstituted phenyl group, or a substituted or unsubstituted silyl group.

The nitrogen-containing compound according to one or more embodiments of the present disclosure may be represented by Formula 1.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIG. 1 is a plan view of a display apparatus according to one or more embodiments of the present disclosure;

FIG. 2 is a cross-sectional view of a display apparatus according to one or more embodiments of the present disclosure;

FIG. 3 is a cross-sectional view schematically showing a light emitting device according to one or more embodiments of the present disclosure;

FIG. 4 is a cross-sectional view schematically showing a light emitting device according to one or more embodiments of the present disclosure;

FIG. 5 is a cross-sectional view schematically showing a light emitting device according to one or more embodiments of the present disclosure;

FIG. 6 is a cross-sectional view schematically showing a light emitting device according to one or more embodiments of the present disclosure;

FIG. 7 and FIG. 8 are each a cross-sectional view of a display apparatuses according to embodiments of the present disclosure;

FIG. 9 is a cross-sectional view showing a display apparatus according to one or more embodiments of the present disclosure;

FIG. 10 is a cross-sectional view showing a display apparatus according to one or more embodiments of the present disclosure; and

FIG. 11 is a perspective view schematically showing an electronic apparatus including a display apparatus according to one or more embodiments.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

Like reference numerals refer to like elements throughout, and duplicative descriptions thereof may not be provided. In the drawings, the dimensions of structures are exaggerated for clarity of illustration. It will be understood that, although the terms first, second, etc. may be utilized herein to describe one or more suitable elements, these elements should not be limited by these terms. These terms are only utilized to distinguish one element from another element. Thus, a first element could be termed a second element without departing from the teachings of the present disclosure. Similarly, a second element could be termed a first element. As utilized herein, the singular forms are intended to include the plural forms as well, unless the context clearly indicates otherwise.

In the description, it will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

In the description, when a layer, a film, a region, a plate, etc. is referred to as being “on” or “above” another part, it can be “directly on” the other part (e.g., without any intervening layers therebetween), or intervening layers may also be present. In contrast, when a layer, a film, a region, a plate, etc. is referred to as being “under” or “below” another part, it can be “directly under” the other part (e.g., without any intervening layers therebetween), or intervening layers may also be present. Also, when an element is referred to as being provided “on” another element, it can be provided under the other element.

As used herein, the singular forms “a,” “an,” and “the” are intended to include the plural forms as well, unless the context clearly indicates otherwise.

As used herein, expressions such as “at least one of”, “one of”, and “selected from”, when preceding a list of elements, modify the entire list of elements and do not modify the individual elements of the list. For example, “at least one selected from (among) a, b and c”, “at least one of a, b or c”, “at least one of a, b and/or c”, “at least one of (among) a-b”, etc., may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” includes any and all combinations of one or more of the associated listed items.

Further, the use of “may” when describing embodiments of the present disclosure refers to “one or more embodiments of the present disclosure”.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic device and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure

In the description, the term “substituted or unsubstituted” corresponds to a group that is unsubstituted or that is substituted with at least one substituent selected from the group consisting of a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the exemplified substituents may be substituted or unsubstituted. For example, a biphenyl group may be interpreted as an aryl group or a phenyl group substituted with a phenyl group.

In the description, the term “forming a ring via the combination with an adjacent group” may refer to forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In some embodiments, the ring formed via the combination with an adjacent group may be combined with another ring to form a spiro structure.

In the description, the term “adjacent group” may refer to a pair of substituent groups where the first substituent is connected to an atom which is directly connected to another atom substituted with the second substituent; a pair of substituent groups connected to the same atom; or a pair of substituent groups where the first substituent is sterically positioned at the nearest position to the second substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In some embodiments, in 4,5-dimethylphenanthrene, two methyl groups may be interpreted as “adjacent groups” to each other.

In the description, a halogen atom may be a fluorine atom, a chlorine atom, a bromine atom, and/or an iodine atom.

In the description, an alkyl group may be a linear, branched or cyclic group. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, an alkenyl group refers to a hydrocarbon group including one or more carbon double bonds in the middle and/or at the terminal of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the description, an alkynyl group refers to a hydrocarbon group including one or more carbon triple bonds in the middle and/or at the terminal of an alkyl group having a carbon number of 2 or more. The alkynyl group may be a linear chain or a branched chain. The carbon number is not specifically limited, but may be 2 to 30, 2 to 20, or 2 to 10. Particular examples of the alkynyl group may include an ethynyl group, a propynyl group, etc., without limitation.

In the description, a hydrocarbon ring group refers to an optional functional group or substituent derived from an aliphatic hydrocarbon ring. The hydrocarbon ring group may be a saturated hydrocarbon ring group of 5 to 20 ring-forming carbon atoms.

In the description, an aryl group refers to an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, a fluorenyl group may be substituted, and two substituents may be combined with each other to form a spiro structure. Examples of a substituted fluorenyl group are as follows, but one or more embodiments of the present disclosure is not limited thereto.

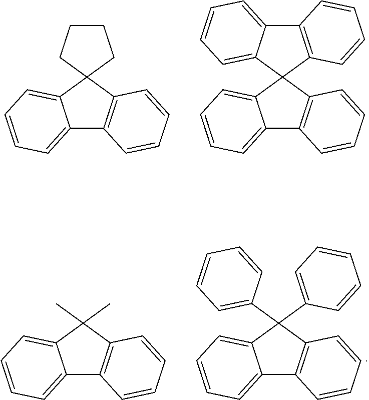

In the description, a heterocyclic group refers to an optional functional group or substituent derived from a ring including one or more selected from among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, when the heterocyclic group includes two or more heteroatoms, two or more heteroatoms may be the same or different. The heterocyclic group may be a monocyclic heterocyclic group or a polycyclic heterocyclic group, and has the concept including a heteroaryl group. The carbon number for forming rings of the heterocyclic group may be 2 to 30, 2 to 20, and 2 to 10.

In the description, an aliphatic heterocyclic group may include one or more selected from among B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, a heteroaryl group may include one or more selected from among B, O, N, P, Si, and S as heteroatoms. When the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 60, 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the same explanation as for the above-described aryl group may be applied to an arylene group except that the arylene group is a divalent group. The same explanation as for the above-described heteroaryl group may be applied to a heteroarylene group except that the heteroarylene group is a a divalent group. Depending on context, a divalent group may refer or be a polyvalent group (e.g., trivalent, tetravalent, etc., and not just divalent) per, e.g., the structure of a formula in connection with which of the terms are utilized.

In the description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, the carbon number of a carbonyl group is not specifically limited, but the carbon number may be 1 to 40, 1 to 30, or 1 to 20. For example, the carbonyl group may have the structures below, but is not limited thereto:

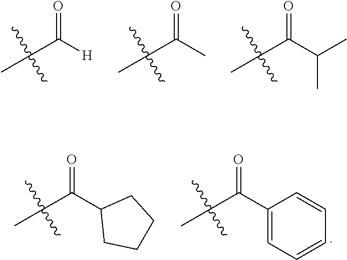

In the description, the carbon number of a sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, a thio group may include an alkyl thio group and an aryl thio group. The thio group may refer to the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may refer to the above-defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, phenoxy, benzyloxy, etc. However, one or more embodiments of the present disclosure is not limited thereto.

In the description, a boron group may refer to the above-defined alkyl group or aryl group, combined with a boron atom. The boron group includes an alkyl boron group and an aryl boron group. Examples of the boron group may include a dimethylboron group, a diethylboron group, a t-butylmethylboron group, a diphenylboron group, a phenylboron group, etc., without limitation.

In the description, an alkenyl group may be a linear chain or a branched chain. The carbon number is not specifically limited, but may be 2 to 30, 2 to 20 or 2 to 10. Examples of the alkenyl group includes a vinyl group, a 1-butenyl group, a 1-pentenyl group, a 1,3-butadienyl aryl group, a styrenyl group, a styrylvinyl group, etc., without limitation.

In the description, the carbon number of the amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation. In some embodiments, the amine group of the description may not refer to a fused ring type or kind of group, but may refer to a chain-type or kind of amine. For example, a ring-type or kind amine group such as a pyrrole group, an imidazole group, a pyridine group, a bipyridine group, a pyrimidine group, a triazine group, an indole group, and/or a carbazole group is defined as a heteroaryl group in the description, and the amine group may refer to only an amine group not forming a ring in the description.

In the description, alkyl groups in an alkylthio group, alkylsulfoxy group, alkylaryl group, alkylamino group, alkyl boron group, alkyl silyl group, and alkyl amine group may be the same as the examples of the above-described alkyl group.

In the description, aryl groups in an aryloxy group, arylthio group, arylsulfoxy group, arylamino group, aryl boron group, aryl silyl group, and aryl amine group may be the same as the examples of the above-described aryl group.

In the description, a direct linkage may refer to a chemical bond (e.g., a single bond).

In some embodiments, in the description,

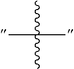

and “ [symbol] ” refer to positions to be connected (e.g., binding site to a corresponding formula).

Hereinafter, embodiments of the present disclosure will be explained referring to the drawings.

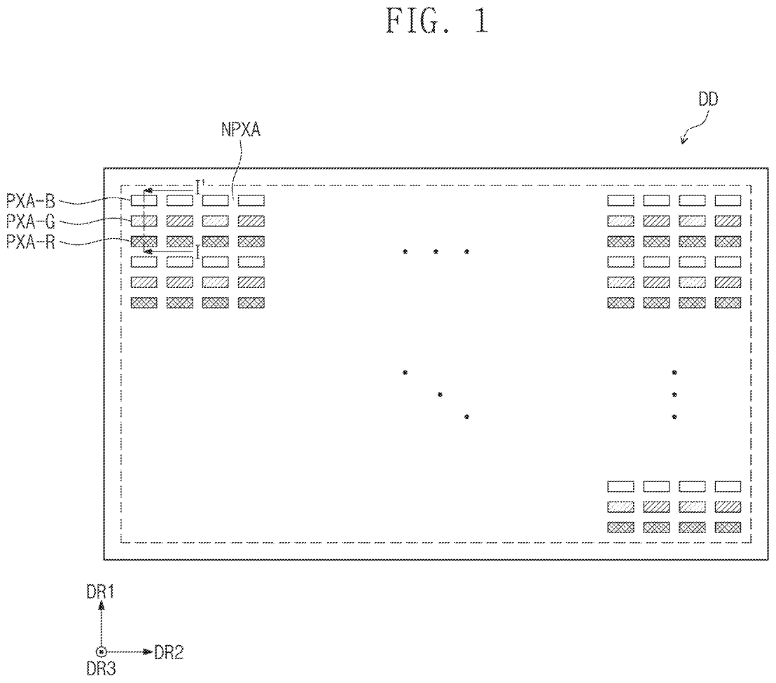

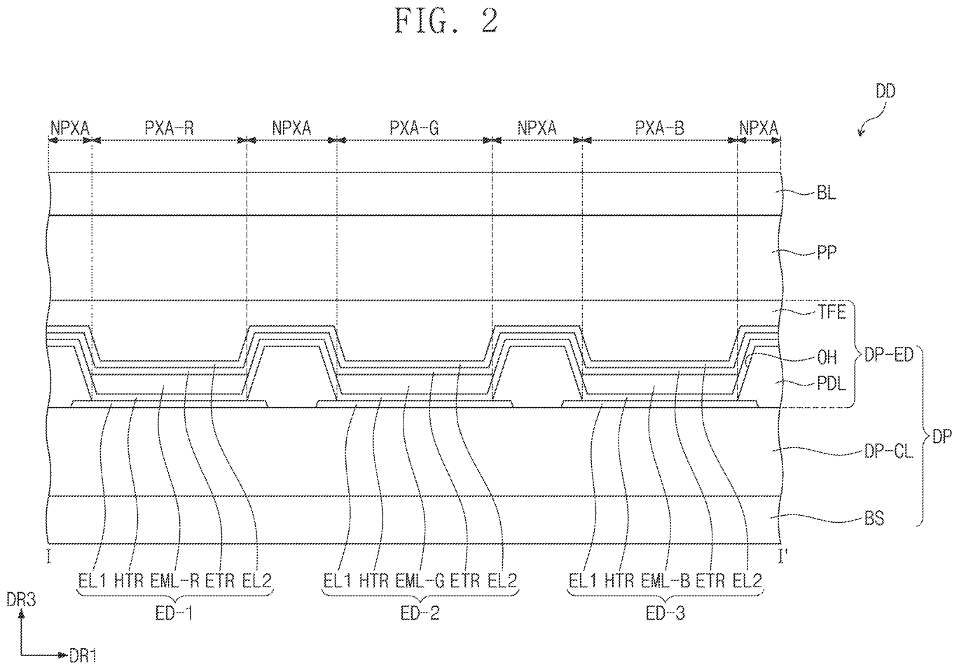

FIG. 1 is a plan view showing one or more embodiments of a display apparatus DD. FIG. 2 is a cross-sectional view of a display apparatus DD of one or more embodiments. FIG. 2 is a cross-sectional view showing a part corresponding to line I-I′ of FIG. 1.

The display apparatus DD may include a display panel DP and an optical layer PP provided on the display panel DP. The display panel DP includes light emitting devices ED-1, ED-2 and ED-3. The display apparatus DD may include multiple light emitting devices ED-1, ED-2 and ED-3. The optical layer PP may be provided on the display panel DP and control reflected light by external light at the display panel DP. The optical layer PP may include, for example, a polarization layer and/or a color filter layer. In some embodiments, different from the drawings, the optical layer PP may not be provided in the display apparatus DD of one or more embodiments.

On the optical layer PP, a base substrate BL may be provided. The base substrate BL may be a member providing a base surface where the optical layer PP is provided. The base substrate BL may be a glass substrate, a metal substrate, a plastic substrate, etc. However, one or more embodiments of the present disclosure is not limited thereto, and the base substrate BL may be an inorganic layer, an organic layer or a composite material layer (e.g., including an organic material and an inorganic material). In some embodiments, the base substrate BL may not be provided.

The display apparatus DD according to one or more embodiments may further include a plugging layer. The plugging layer may be provided between a display device layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL provided on the base layer BS and a display device layer DP-ED. The display device layer DP-ED may include a pixel definition layer PDL, light emitting devices ED-1, ED-2 and ED-3 provided in the pixel definition layer PDL, and an encapsulating layer TFE provided on the light emitting devices ED-1, ED-2 and ED-3.

The base layer BS may be a member providing a base surface where the display device layer DP-ED is provided. The base layer BS may be a glass substrate, a metal substrate, a plastic substrate, etc. However, one or more embodiments of the present disclosure is not limited thereto, and the base layer BS may be an inorganic layer, an organic layer or a composite material layer.

In one or more embodiments, the circuit layer DP-CL is provided on the base layer BS, and the circuit layer DP-CL may include one or more transistors. Each of the transistors may include a control electrode, an input electrode, and an output electrode. For example, the circuit layer DP-CL may include switching transistor(s) and driving transistor(s) for driving the light emitting devices ED-1, ED-2 and ED-3 of the display device layer DP-ED.

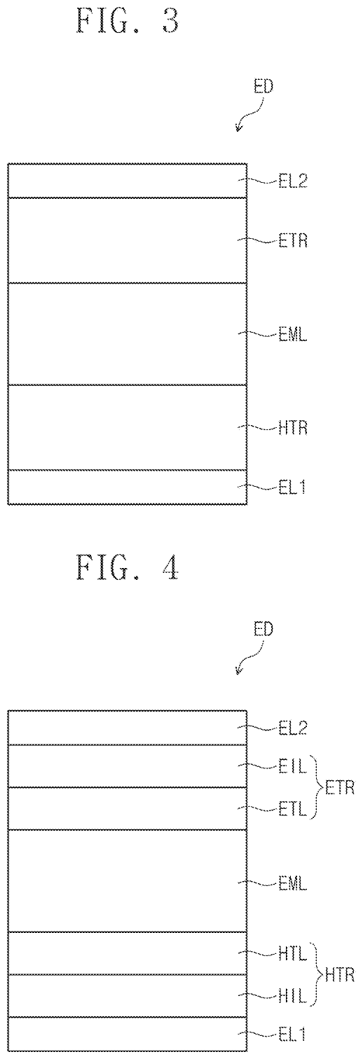

Each of the light emitting devices ED-1, ED-2 and ED-3 may have the structures of light emitting devices ED of embodiments according to FIG. 3 to FIG. 6, which will be explained in more detail herein below. Each of the light emitting devices ED-1, ED-2 and ED-3 may include a first electrode EL1, a hole transport region HTR, emission layers EML-R, EML-G and EML-B, an electron transport region ETR, and a second electrode EL2.

In FIG. 2, shown is an embodiment where the emission layers EML-R, EML-G and EML-B of light emitting devices ED-1, ED-2 and ED-3 are provided in opening portions OH defined in a pixel definition layer PDL, and a hole transport region HTR, an electron transport region ETR and a second electrode EL2 are provided as common layers in all light emitting devices ED-1, ED-2 and ED-3. However, one or more embodiments of the present disclosure is not limited thereto. In one or more embodiments, the hole transport region HTR and the electron transport region ETR may be patterned and provided in the opening portions OH defined in the pixel definition layer PDL. For example, in one or more embodiments, the hole transport region HTR, the emission layers EML-R, EML-G and EML-B, and the electron transport region ETR of the light emitting devices ED-1, ED-2 and ED-3 may be patterned by an ink jet printing method and provided.

An encapsulating layer TFE may cover the light emitting devices ED-1, ED-2 and ED-3. The encapsulating layer TFE may encapsulate the display device layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to one or more embodiments may include at least one inorganic layer (hereinafter, encapsulating inorganic layer). In some embodiments, the encapsulating layer TFE according to one or more embodiments may include at least one organic layer (hereinafter, encapsulating organic layer) and at least one encapsulating inorganic layer.

The encapsulating inorganic layer protects the display device layer DP-ED from moisture/oxygen, and the encapsulating organic layer protects the display device layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be provided on the second electrode EL2 and may be provided while filling the opening portion OH.

Referring to FIG. 1 and FIG. 2, the display apparatus DD may include a non-luminous area NPXA and luminous areas PXA-R, PXA-G and PXA-B. The luminous areas PXA-R, PXA-G and PXA-B may be areas emitting (e.g., configured to emit) light produced from the light emitting devices ED-1, ED-2 and ED-3, respectively. The luminous areas PXA-R, PXA-G and PXA-B may be separated from each other on a plane (e.g., in a plan view).

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, in the disclosure, each of the luminous areas PXA-R, PXA-G and PXA-B may correspond to each pixel. The pixel definition layer PDL may divide the light emitting devices ED-1, ED-2 and ED-3. The emission layers EML-R, EML-G and EML-B of the light emitting devices ED-1, ED-2 and ED-3 may be provided and divided in the opening portions OH defined in the pixel definition layer PDL.

The luminous areas PXA-R, PXA-G and PXA-B may be divided into multiple groups according to the color of light produced from the light emitting devices ED-1, ED-2 and ED-3. In the display apparatus DD of one or more embodiments, shown in FIG. 1 and FIG. 2, three luminous areas PXA-R, PXA-G and PXA-B emitting (e.g., configured to emit) red light, green light and blue light are illustrated as one or more embodiments. For example, the display apparatus DD of one or more embodiments may include a red luminous area PXA-R, a green luminous area PXA-G and a blue luminous area PXA-B, which are separated from each other.

In the display apparatus DD according to one or more embodiments, multiple light emitting devices ED-1, ED-2 and ED-3 may be to emit light having different wavelength regions. For example, in one or more embodiments, the display apparatus DD may include a first light emitting device ED-1 emitting (e.g., configured to emit) red light, a second light emitting device ED-2 emitting (e.g., configured to emit) green light, and a third light emitting device ED-3 emitting (e.g., configured to emit) blue light. For example, each of the red luminous area PXA-R, the green luminous area PXA-G, and the blue luminous area PXA-B of the display apparatus DD may respectively correspond to the first light emitting device ED-1, the second light emitting device ED-2, and the third light emitting device ED-3.

However, one or more embodiments of the present disclosure is not limited thereto, and the first to third light emitting devices ED-1, ED-2 and ED-3 may be to emit light in substantially the same wavelength region, or at least one thereof may be to emit light in a different wavelength region. For example, all of the first to third light emitting devices ED-1, ED-2 and ED-3 may be to emit blue light.

The luminous areas PXA-R, PXA-G and PXA-B in the display apparatus DD according to one or more embodiments may be arranged in a stripe shape (stripe pattern). Referring to FIG. 1, one or more red luminous areas PXA-R may be arranged with each other along a second directional axis DR2, one or more green luminous areas PXA-G may be arranged with each other along a second directional axis DR2, and one or more blue luminous areas PXA-B may be arranged with each other along a second directional axis DR2. In some embodiments, the red luminous area PXA-R, the green luminous area PXA-G and the blue luminous area PXA-B may be arranged alternatingly with each other along a first directional axis DR1.

In FIG. 1 and FIG. 2, the areas of the luminous areas PXA-R, PXA-G and PXA-B are shown to be similar, but one or more embodiments of the present disclosure is not limited thereto. The areas of the luminous areas PXA-R, PXA-G and PXA-B may be different from each other according to the wavelength region of light emitted. In some embodiments, the areas of the luminous areas PXA-R, PXA-G and PXA-B may refer to areas on a plane defined by the first directional axis DR1 and the second directional axis DR2.

In some embodiments, the arrangement of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in one or more suitable combinations according to the properties of display quality required or desired for the display apparatus DD. For example, the arrangement of the luminous areas PXA-R, PXA-G and PXA-B may be a pentile (PENTILE®) arrangement, or a diamond (Diamond Pixel™) arrangement (PENTILE® is a registered trademark owned by Samsung Display Co., Ltd.).

In some embodiments, the areas of the luminous areas PXA-R, PXA-G and PXA-B may be different from each other. For example, in one or more embodiments, the area of the green luminous area PXA-G may be smaller than the area of the blue luminous area PXA-B, but one or more embodiments of the present disclosure is not limited thereto.

Hereinafter, FIG. 3 to FIG. 6 are cross-sectional views schematically showing light emitting devices according to embodiments. The light emitting device ED according to one or more embodiments may include a first electrode EL1, a hole transport region HTR, an emission layer EML, an electron transport region ETR and a second electrode EL2, stacked in order.

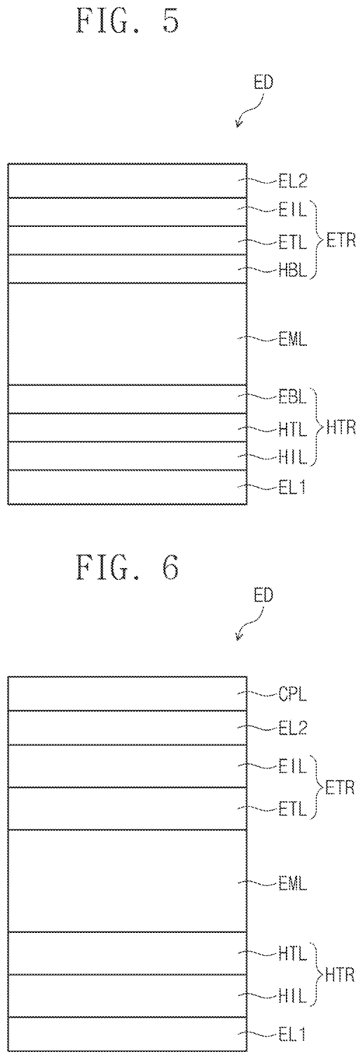

When compared to FIG. 3, FIG. 4 shows the cross-sectional view of a light emitting device ED of one or more embodiments, wherein a hole transport region HTR includes a hole injection layer HIL and a hole transport layer HTL, and an electron transport region ETR includes an electron injection layer EIL and an electron transport layer ETL. In some embodiments, when compared to FIG. 3, FIG. 5 shows the cross-sectional view of a light emitting device ED of one or more embodiments, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared to FIG. 4, FIG. 6 shows the cross-sectional view of a light emitting device ED of one or more embodiments, including a capping layer CPL provided on the second electrode EL2.

The first electrode EL1 has conductivity (e.g., is a conductor). The first electrode EL1 may be formed utilizing a metal material, a metal alloy or any suitable conductive compound. The first electrode EL1 may be an anode or a cathode. However, one or more embodiments of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (stacked structure of Li and F), LiF/Al (stacked structure of Li and Al), Mo, Ti, W, compounds thereof, and/or mixtures thereof (for example, a mixture of Ag and Mg). Also, the first electrode EL1 may have a structure including multiple layers including a reflective layer and/or a transflective layer formed utilizing any of the above materials, and a transmissive conductive layer formed utilizing ITO, IZO, ZnO, and/or ITZO. For example, the first electrode EL1 may have a three-layer structure of ITO/Ag/ITO. However, one or more embodiments of the present disclosure is not limited thereto. The first electrode EL1 may include any of the above-described metal materials, combinations of two or more metal materials selected from among the above-described metal materials, and/or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission auxiliary layer, or an emission blocking layer EBL. The thickness of the hole transport region HTR may be, for example, about 50 Å to about 15,000 Å.

The hole transport region HTR may have a single layer formed utilizing a single material, a single layer formed utilizing multiple different materials, or a multilayer structure including multiple layers formed utilizing multiple different materials.

For example, the hole transport region HTR may have the structure of a single layer of a hole injection layer HIL or a hole transport layer HTL, and may have a structure of a single layer formed utilizing a hole injection material and a hole transport material. In some embodiments, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/buffer layer, hole transport layer HTL/buffer layer, or hole injection layer HIL/hole transport layer HTL/electron blocking layer EBL, without limitation.

The hole transport region HTR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include a compound represented by Formula H-2.

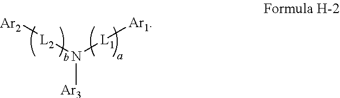

In Formula H-2 above, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. “a” and “b” may each independently be an integer of 0 to 10. In some embodiments, when “a” or “b” is an integer of 2 or more, multiple L₁ and L₂ may each independently be a substituted or unsubstituted arylene group of 6 to 30 ring-forming carbon atoms, or a substituted or unsubstituted heteroarylene group of 2 to 30 ring-forming carbon atoms.

In Formula H-2, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-2, Ar₃ may be a substituted or unsubstituted aryl group of 6 to 30 ring-forming carbon atoms or a substituted or unsubstituted heteroaryl group of 2 to 30 ring-forming carbon atoms.

The compound represented by Formula H-2 may be a monoamine compound. In some embodiments, the compound represented by Formula H-2 may be a diamine compound in which at least one selected from among Ar₁ to Ar₃ includes an amine group as a substituent. In some embodiments, the compound represented by Formula H-2 may be a carbazole-based compound in which at least one selected from among Ar₁ and Ar₂ includes a substituted or unsubstituted carbazole group, and/or a fluorene-based compound in which at least one selected from among Ar₁ and Ar₂ includes a substituted or unsubstituted fluorene group.

The compound represented by Formula H-2 may be represented by any one selected from among the compounds in Compound Group H. However, the compounds shown in Compound Group H are only illustrations, and the compound represented by Formula H-2 is not limited to the compounds represented in Compound Group H.

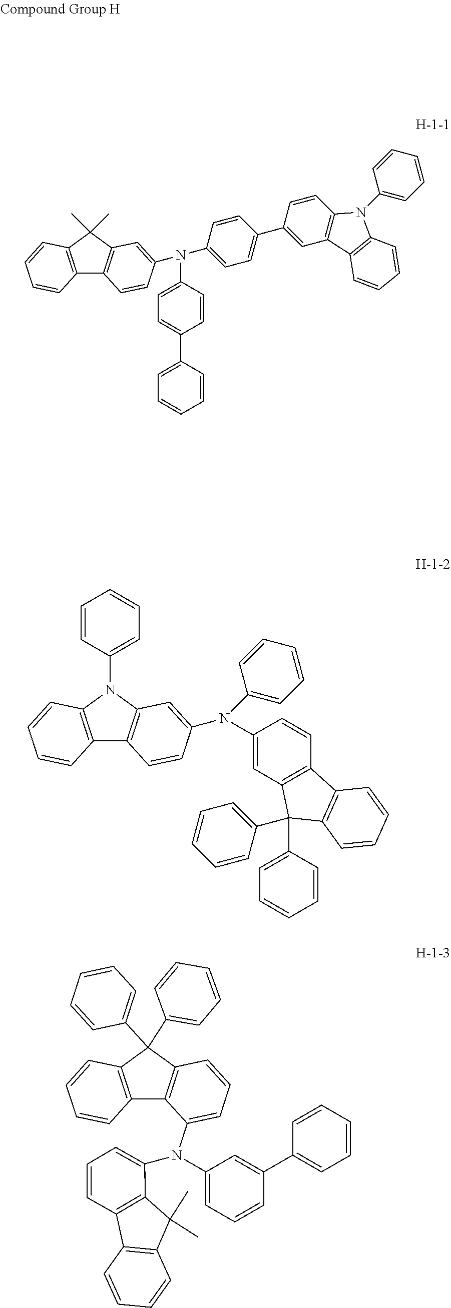 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

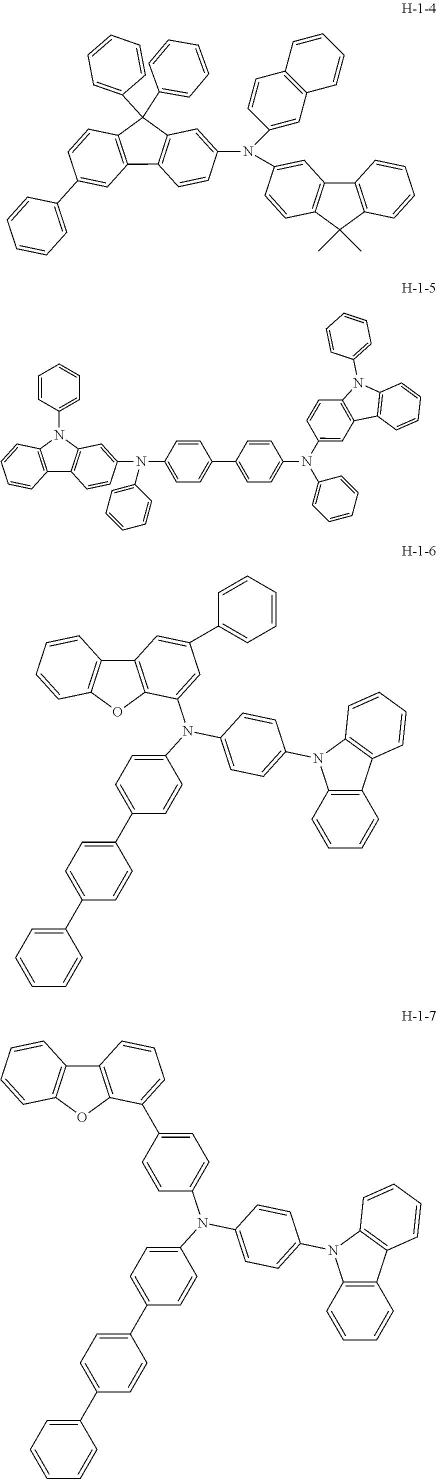 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

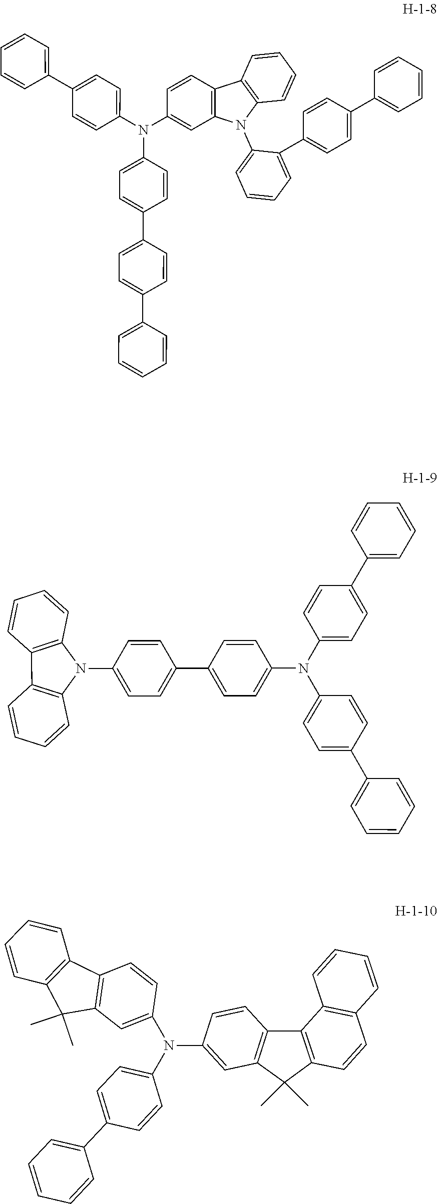 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

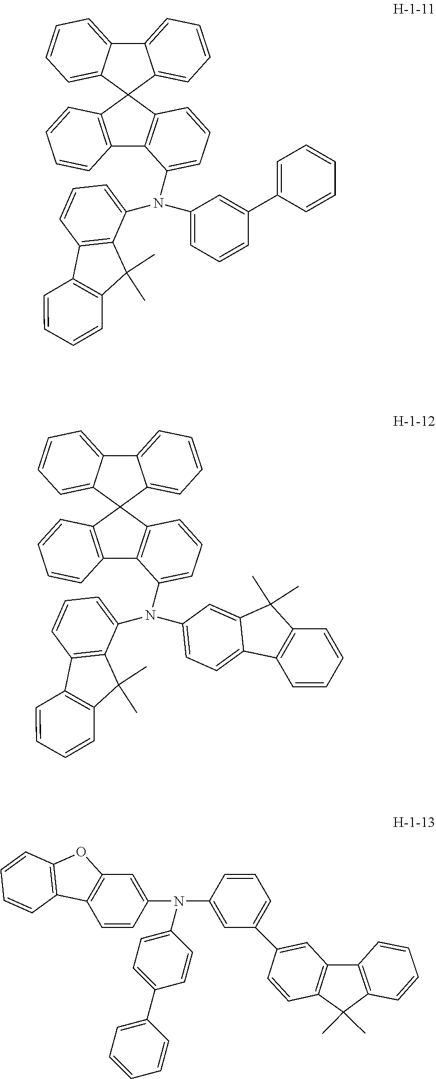 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

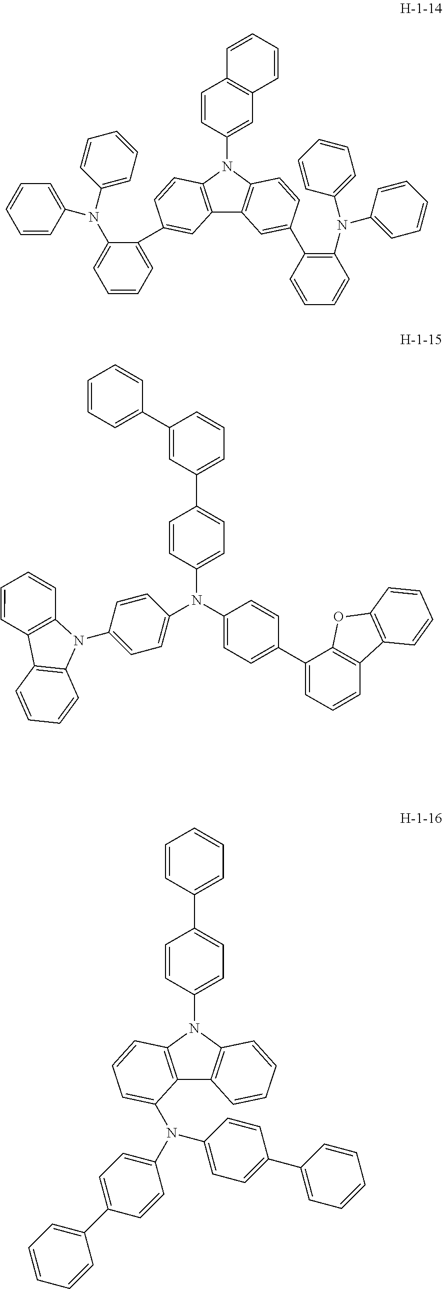 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

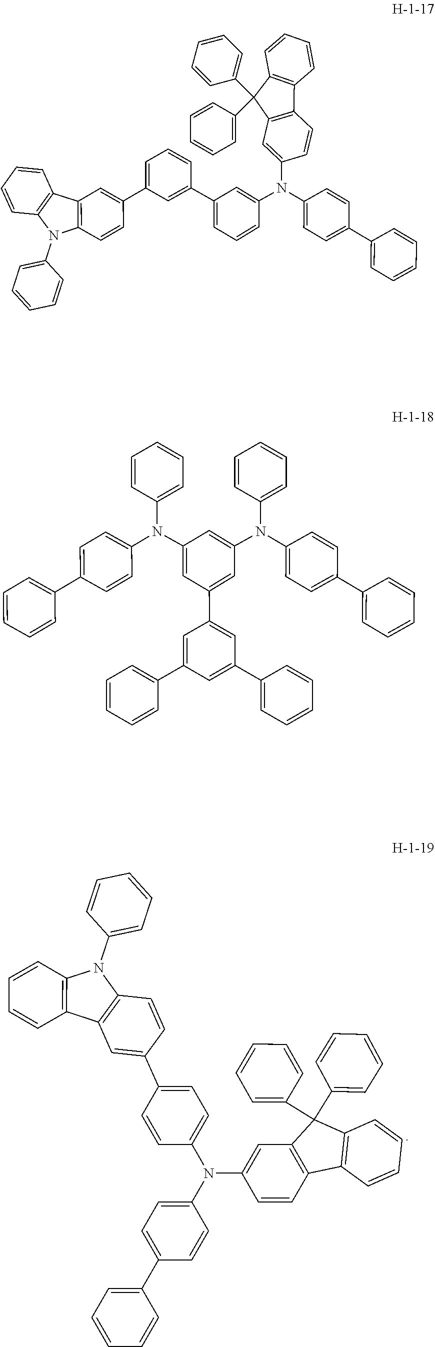 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound (such as copper phthalocyanine), N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HATCN).

The hole transport region HTR may include carbazole derivatives (such as N-phenyl carbazole and/or polyvinyl carbazole), fluorene-based derivatives, N,N′-bis(3-methylphenyl)-N,N′-diphenyl-[1,1′-biphenyl]-4,4′-diamine (TPD), triphenylamine-based derivatives (such as 4,4′,4″-tris(N-carbazolyl)triphenylamine (TCTA)), N,N′-di(1-naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one selected from among the hole injection layer HIL, hole transport layer HTL, and electron blocking layer EBL.

The thickness of the hole transport region HTR may be from about 100 Å to about 10,000 Å, for example, from about 100 Å to about 5,000 Å. When the hole transport region HTR includes a hole injection layer HIL, the thickness of the hole injection region HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy any of their respective above-described ranges, satisfactory or suitable hole transport properties may be achieved without substantial increase of a driving voltage.

The hole transport region HTR may further include a charge generating material to increase conductivity, in addition to the above-described materials. The charge generating material may be dispersed substantially uniformly or non-substantially uniformly in the hole transport region HTR. The charge generating material may be, for example, a p-dopant. The p-dopant may include at least one of metal halide compounds, quinone derivatives, metal oxides, or cyano group-containing compounds, without limitation. For example, the p-dopant may include metal halide compounds (such as CuI and/or RbI), quinone derivatives (such as tetracyanoquinodimethane (TCNQ) and/or 2,3,5,6-tetrafluoro-7,7′,8,8-tetracyanoquinodimethane (F4-TCNQ)), metal oxides (such as tungsten oxide and/or molybdenum oxide), cyano group-containing compounds (such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9)), etc., without limitation.

As described above, the hole transport region HTR may further include at least one selected from among a buffer layer and an electron blocking layer EBL, in addition to the hole injection layer HIL and the hole transport layer HTL. The buffer layer may compensate resonance distance according to the wavelength of light emitted from an emission layer EML and may increase emission efficiency. As materials included in the buffer layer, materials which may be included in the hole transport region HTR may be utilized. The electron blocking layer EBL is a layer playing the role of blocking or reducing the injection of electrons from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing multiple different materials, or a multilayer structure having multiple layers formed utilizing multiple different materials.

In the light emitting element ED according to one or more embodiments, the emission layer EML may include the nitrogen-containing compound of one or more embodiments. In one or more embodiments, the emission layer EML may include the nitrogen-containing compound of one or more embodiments as a host. The nitrogen-containing compound of one or more embodiments may be the host material of the emission layer EML. In some embodiments, the nitrogen-containing compound may be referred to as a first compound.

The nitrogen-containing compound of one or more embodiments includes a first carbazole group and a second carbazole group connected with the first carbazole group. The first carbazole group may include first and second benzene moieties, and the second carbazole group may include third and fourth benzene moieties. The nitrogen atom of the second carbazole group may be connected with the first benzene moiety of the first carbazole group.

The nitrogen-containing compound of one or more embodiments includes at least one first substituent. The first substituent includes a phenyl moiety or a triphenyl silane moiety. In the nitrogen-containing compound of one or more embodiments, the first substituent is connected with the first carbazole group or the second carbazole group. For example, the first substituent of the nitrogen-containing compound of one or more embodiments is bonded at carbon position 4 of the first carbazole group or at carbon position 4 of the second carbazole group. For example, the second carbazole group may be connected with the first benzene moiety of the first carbazole group, and the first substituent may be connected with the second benzene moiety of the first carbazole group. In some embodiments, the first substituent may be bonded to the third benzene moiety or the fourth benzene moiety of the second carbazole group. In some embodiments, multiple first substituents may be provided, and the multiple first substituents may be bonded to the first carbazole group and the second carbazole group, respectively. For example, in the nitrogen-containing compound, the first substituent is connected with the first and/or second carbazole groups, and when the nitrogen-containing compound of one or more embodiments is applied to a light emitting device, high efficiency and long lifetime may be achieved.

The numbering of carbon atoms constituting the first carbazole group and the second carbazole group is shown in Formula S1.

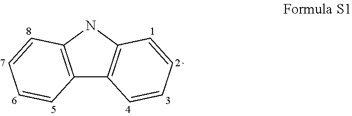 [SMILES: c1ccc2c(c1)[nH]c1ccccc12]

The numbering of the carbon of the carbazole group starts from a carbon atom at a position adjacent to a nitrogen atom clockwise and in order as shown in Formula S1. For the convenience of explanation, substituents to be connected with the nitrogen atom are omitted in Formula S1.

The nitrogen-containing compound of one or more embodiments may be represented by Formula 1.

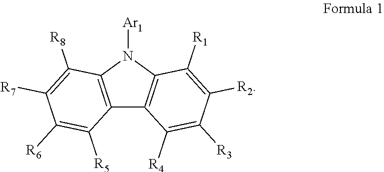

In Formula 1, Ar₁ is a substituted or unsubstituted aryl group of 6 to 30 ring-forming carbon atoms, or a substituted or unsubstituted heteroaryl group of 2 to 30 ring-forming carbon atoms. For example, Ar₁ may be a substituted or unsubstituted phenyl group, a substituted or unsubstituted biphenyl group, a substituted or unsubstituted terphenyl group, a substituted or unsubstituted quaterphenyl group, a substituted or unsubstituted quinquephenyl group, a substituted or unsubstituted carbazole group, a substituted or unsubstituted dibenzofuran group, or a substituted or unsubstituted dibenzothiophene group.

In Formula 1, R₁ to R₈ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₁ to R₈ may each independently be a hydrogen atom, a deuterium atom, a substituted or unsubstituted phenyl group, a substituted or unsubstituted biphenyl group, a substituted or unsubstituted silyl group, or a substituted or unsubstituted carbazole group.

In Formula 1, any one selected from among R₅ to R₈ is a substituent represented by Formula 2.

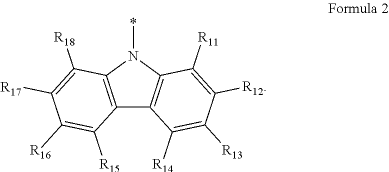

In Formula 2, “ [symbol] ” is a position connected with a corresponding carbon atom in Formula 1.

In Formula 2, R₁₁ to R₁₈ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₁₁ to R₁₈ may each independently be a hydrogen atom, a deuterium atom, a substituted or unsubstituted phenyl group, a substituted or unsubstituted biphenyl group, a substituted or unsubstituted silyl group, or a substituted or unsubstituted carbazole group.

In Formula 1 and Formula 2, at least one selected from among R₄ and R₁₄ is a substituted or unsubstituted phenyl group or a substituent represented by Formula 3.

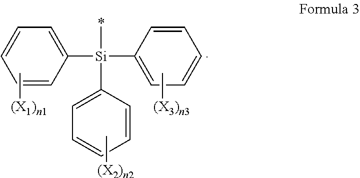

In Formula 3, “ [symbol] ” is a position connected with a corresponding carbon [symbol] atom of Formula 1 and/or Formula 2.

In Formula 3, X₁ to X₃ may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In some embodiments, X₁ to X₃ may each independently be combined with an adjacent group to form a ring. For example, X₁ to X₃ may each independently be a hydrogen atom, a deuterium atom, a substituted or unsubstituted phenyl group, a substituted or unsubstituted carbazole group, or a substituted or unsubstituted dibenzofuran group. In some embodiments, when n1 is an integer of 2 or more, one X₁ selected from among multiple X₁ may correspond to a substituted or unsubstituted amine group, another X₁ adjacent to the one X₁ selected from among multiple X₁ may correspond to a substituted or unsubstituted phenyl group, and the one X₁ and another X₁ may be combined with each other to provide a substituted or unsubstituted carbazole group together with a benzene moiety connected with the silicon atom of Formula 3. In some embodiments, when n1 is an integer of 2 or more, one X₁ selected from among multiple X₁ may correspond to a substituted or unsubstituted oxy group, another X₁ adjacent to the one X₁ selected from among multiple X₁ may correspond to a substituted or unsubstituted phenyl group, and the one X₁ and another X₁ may be combined with each other to provide a substituted or unsubstituted dibenzofuran group together with a benzene moiety connected with the silicon atom of Formula 3. In some embodiments, when n1 is an integer of 2 or more, one X₁ selected from among multiple X₁ may correspond to a substituted or unsubstituted thio group, another X₁ adjacent to the one X₁ selected from among multiple X₁ may correspond to a substituted or unsubstituted phenyl group, and the one X₁ and another X₁ may be combined with each other to provide a substituted or unsubstituted dibenzothiophene group together with a benzene moiety connected with the silicon atom of Formula 3.

In Formula 3, n1 to n3 may each independently be an integer of 0 to 5. In Formula 3, when n1 to n3 are 0, the nitrogen-containing compound of one or more embodiments may be unsubstituted with X₁ to X₃, respectively. In Formula 3, a case where n1 to n3 are 5, and X₁ to X₃ are all hydrogen atoms, may be the same as a case where n1 to n3 are 0 in Formula 3. When n1 to n3 are integers of 2 or more, each of multiple corresponding X₁ to X₃ may be all the same, or at least one selected from among multiple corresponding X₁ to X₃ may be different.

In some embodiments, Formula 1 may correspond to the first carbazole group, and Formula 2 may correspond to the second carbazole group. In some embodiments, the substituted or unsubstituted phenyl group connected with (or as) R₄ of Formula 1 and/or R₁₄ of Formula 2, and/or Formula 3 may correspond to the first substituent. The R₄ of Formula 1 and/or R₁₄ of Formula 2 may correspond to (e.g., may be attached to) the carbon of position 4 of the first or second carbazole group.

In one or more embodiments, the first compound represented by Formula 1 may be represented by Formula 1-1.

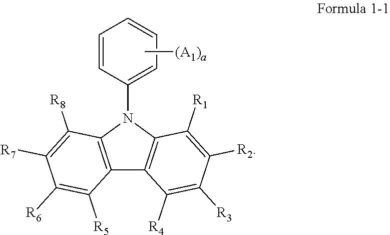

Formula 1-1 represents Formula 1 wherein Ar₁ is specified. Formula 1-1 represents a case where the substituent connected with the nitrogen atom of the first carbazole group is a substituted or unsubstituted phenyl group.

In Formula 1-1, A₁ may be a hydrogen atom, a deuterium atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl, a substituted or unsubstituted biphenyl group. In some embodiments, in Formula 1-1, A₁ may be combined with an adjacent group to form a ring. For example, A₁ may be a hydrogen atom, a deuterium atom, a substituted or unsubstituted phenyl group, a substituted or unsubstituted biphenyl group, a substituted or unsubstituted carbazole group, or a substituted or unsubstituted silyl group. In some embodiments, when “a” is an integer of 2 or more, one A₁ selected from among multiple A₁ may correspond to a substituted or unsubstituted oxy group, another A₁ adjacent to the one A₁ selected from among multiple A₁ may correspond to a substituted or unsubstituted phenyl group, and the one A₁ and another A₁ may be combined with each other to provide a substituted or unsubstituted dibenzofuran group together with a benzene moiety connected with the nitrogen atom of Formula 1-1.

In Formula 1-1, “a” is an integer of 0 to 5. In Formula 1-1, when “a” is 0, the nitrogen-containing compound of one or more embodiments may be unsubstituted with A₁. A case of Formula 1-1 where “a” is 5, and all A₁ are hydrogen atoms, may be the same as Formula 1-1 where “a” is 0. When “a” is an integer of 2 or more, multiple A₁ may be all the same, or at least one selected from among multiple A₁ may be different.

In some embodiments, in Formula 1-1, the same descriptions as those provided in connection with Formula 1 may be applied for R₁ to R₈.

In Formula 1-1, A₁ may be a substituent represented by any one selected from among Formula 4-1 to Formula 4-11.

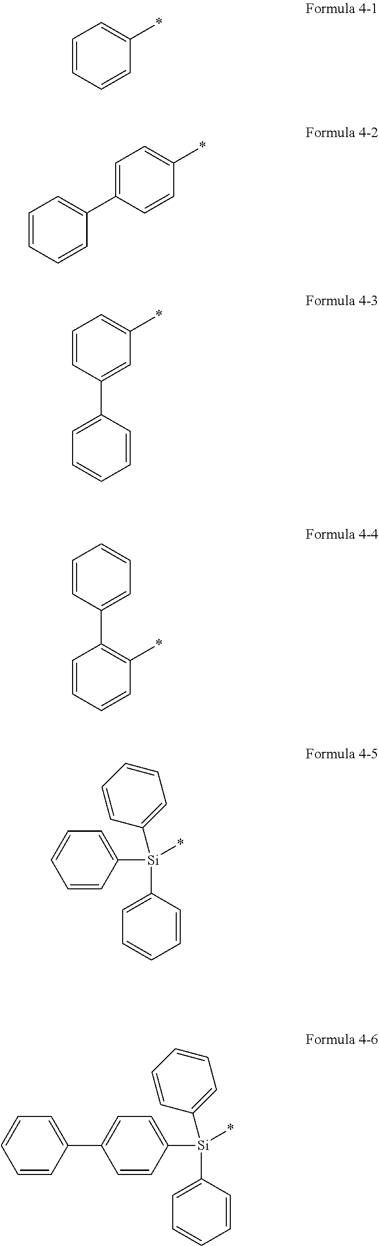 [SMILES: *c1ccccc1 ; *c1ccc(-c2ccccc2)cc1 ; *c1cccc(-c2ccccc2)c1 ; *c1ccccc1-c1ccccc1 ; *[Si](c1ccccc1)(c1ccccc1)c1ccccc1 ; *[Si](c1ccccc1)(c1ccccc1)c1ccc(-c2ccccc2)cc1]

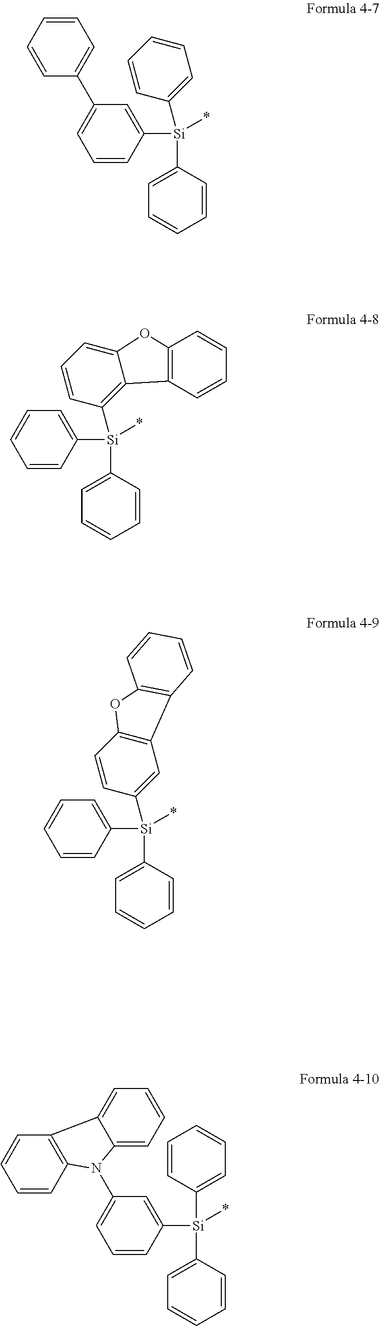 [SMILES: *[Si](c1ccccc1)(c1ccccc1)c1cccc(-c2ccccc2)c1 ; *[Si](c1ccccc1)(c1ccccc1)c1cccc2oc3ccccc3c12 ; *[Si](c1ccccc1)(c1ccccc1)c1ccc2oc3ccccc3c2c1 ; *[Si](c1ccccc1)(c1ccccc1)c1cccc(-n2c3ccccc3c3ccccc32)c1]

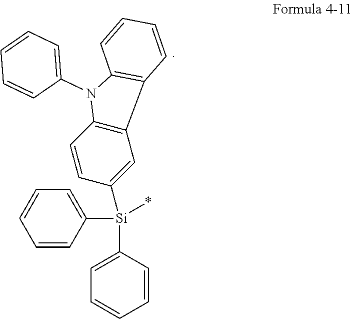 [SMILES: *[Si](c1ccccc1)(c1ccccc1)c1ccc2c(c1)c1ccccc1n2-c1ccccc1]

In Formula 4-1 to Formula 4-11, “ [symbol] ” is a position connected with Formula 1-1.

In one or more embodiments, the first compound represented by Formula 1 may be represented by Formula 1-2.

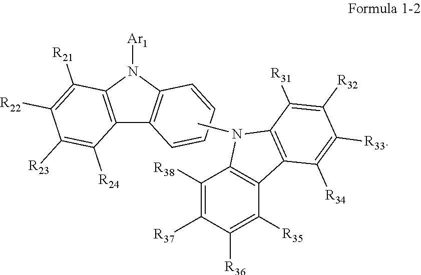

Formula 1-2 represents Formula 1 where some of R₅ to R₈ are specified. Formula 1-2 represents a case where the second carbazole group is connected with the first benzene moiety of the first carbazole group, and other substituents connected with the first benzene moiety are hydrogen atoms.

In Formula 1-2, R₂₁ to R₃₈ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₂₁ to R₃₈ may each independently be a hydrogen atom, a deuterium atom, a substituted or unsubstituted phenyl group, a substituted or unsubstituted biphenyl group, a substituted or unsubstituted silyl group, or a substituted or unsubstituted carbazole group.

In Formula 1-2, at least one selected from among R₂₄ and R₃₄ may be a substituted or unsubstituted phenyl group, or a substituent represented by Formula 3.

In some embodiments, in Formula 1-2, the same descriptions as those provided in connection with Formula 1 may be applied for Ar₁.

In one or more embodiments, the substituent represented by Formula 3 may be represented by Formula 3-1.

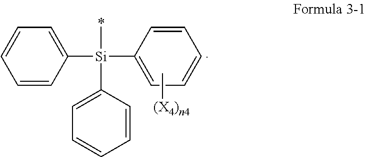

Formula 3-1 represents Formula 3 where X₁ to X₃ are specified. For example, Formula 3-1 represents a case of Formula 3 where at least two selected from among X₁ to X₃ are hydrogen atoms.

In Formula 3-1, X₄ may be a hydrogen atom, a deuterium atom, a cyano group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl group, or a substituted or unsubstituted heteroaryl group of 2 to 30 ring-forming carbon atoms. In some embodiments, in Formula 3-1, X₄ may be combined with an adjacent group to form a ring. For example, X₄ may be a hydrogen atom, a deuterium atom, a substituted or unsubstituted phenyl group or a substituted or unsubstituted carbazole group.

In Formula 3-1, n4 is an integer of 0 to 5. In Formula 3-1, when n4 is 0, the nitrogen-containing compound of one or more embodiments may be unsubstituted with X₄. A case of Formula 3-1 where n4 is 5, and all X₄ are hydrogen atoms, may be the same as a case of Formula 3-1 where n4 is 0. When n4 is an integer of 2 or more, multiple X₄ may be all the same, or at least one selected from among multiple X₄ may be different.

In one or more embodiments, the first compound represented by Formula 1 may be represented by Formula 1-3 or Formula 1-4.

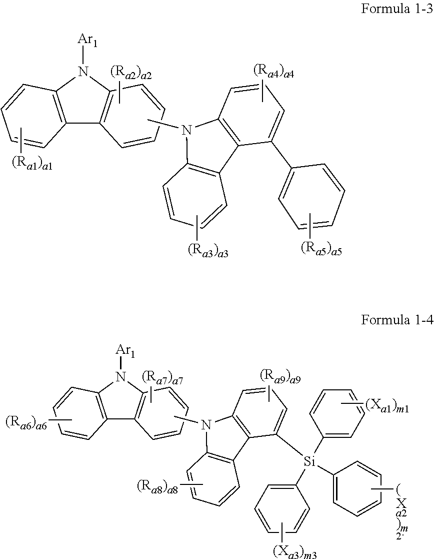

Formula 1-3 and Formula 1-4 represent a case of Formula 1 where R₁₄ of Formula 2 is a substituted or unsubstituted phenyl group and a case of Formula 1 where R₁₄ of Formula 2 is the substituent represented by Formula 3, respectively. Formula 1-3 and Formula 1-4 represent cases where the first substituent is bonded to the carbon of position 4 of the second carbazole group.

In Formula 1-3 and Formula 1-4, Rₐ₁ to Rₐ₉ may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Rₐ₁ to Rₐ₉ may each independently be a hydrogen atom, a deuterium atom, a substituted or unsubstituted phenyl group, a substituted or unsubstituted silyl group, or a substituted or unsubstituted carbazole group. For example, Rₐ₅ may be a hydrogen atom, a deuterium atom, or a substituted or unsubstituted phenyl group.

In Formula 1-4, Xₐ₁ to Xₐ₃ may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In some embodiments, in Formula 1-4, Xₐ₁ to Xₐ₃ may each independently be combined with an adjacent group to form a ring. For example, Xₐ₁ to Xₐ₃ may each independently be a hydrogen atom, a deuterium atom, or a substituted or unsubstituted phenyl group.

In Formula 1-3, a1 and a3 may each independently be an integer of 0 to 4, a2 and a4 may each independently be an integer of 0 to 3, and a5 may be an integer of 0 to 5. In Formula 1-3, when a1 to a5 are 0, the nitrogen-containing compound of one or more embodiments may be unsubstituted with Rₐ₁ to Rₐ₅, respectively. In Formula 1-3, a case where a1 and a3 are 4, a2 and a4 are 3, a5 is 5, and Rₐ₁ to Rₐ₅ are hydrogen atoms, may be the same as a case of Formula 1-3 where a1 to a5 are 0. In Formula 1-3, when a1 to a5 are integers of 2 or more, each of multiple Rₐ₁ to Rₐ₅ may be all the same, or at least one selected from among Rₐ₁ to Rₐ₅ may be different.

In Formula 1-4, a6 and a8 may each independently be an integer of 0 to 4, a7 and a9 may each independently be an integer of 0 to 3, and m1 to m3 may each independently be an integer of 0 to 5. In Formula 1-4, when a6 to a9 and m1 to m3 are 0, the nitrogen-containing compound of one or more embodiments may be unsubstituted with Rₐ₆ to Rₐ₉ and Xₐ₁ to Xₐ₃, respectively. In Formula 1-4, a case where a6 and a8 are 4, a7 and a9 are 3, m1 to m3 are 5, Rₐ₆ to Rₐ₉, and Xₐ₁ to Xₐ₃ are all hydrogen atoms, may be the same as a case of Formula 1-4 where a6 to a9 and m1 to m3 are 0. In Formula 1-4, when a6 to a9 and m1 to m3 are integers of 2 or more, each of multiple Rₐ₆ to Rₐ₉, and Xₐ₁ to Xₐ₃ may be all the same, or at least one selected from among multiple Rₐ₆ to Rₐ₉, and Xₐ₁ to Xₐ₃ may be different.

In some embodiments, in Formula 1-3 and Formula 1-4, the same descriptions as those provided in connection with Formula 1 may be applied for Ar₁.

In one or more embodiments, the first compound represented by Formula 1 may be represented by Formula 1-5 or Formula 1-6.

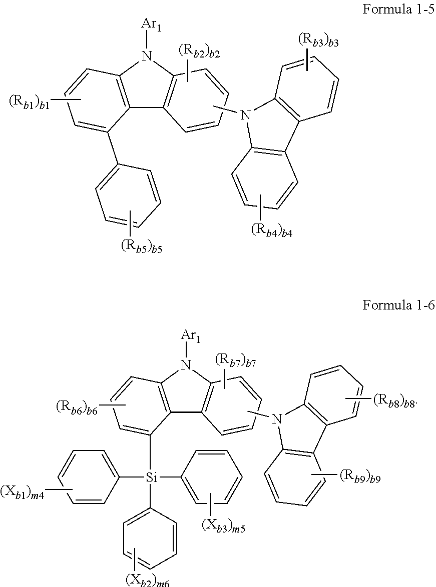

Formula 1-5 and Formula 1-6 represent a case of Formula 1 where R₄ of Formula 1 is a substituted or unsubstituted phenyl group, and a case of Formula 1 where R₄ of Formula 1 is the substituent represented by Formula 3, respectively. Formula 1-5 and Formula 1-6 represent cases where the first substituent is bonded to the carbon of position 4 of the first carbazole group.

In Formula 1-5 and Formula 1-6, R_{b1} to R_{b9} may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R_{b1} to R_{b9} may each independently be a hydrogen atom, a deuterium atom, a substituted or unsubstituted phenyl group, a substituted or unsubstituted silyl group, or a substituted or unsubstituted carbazole group. For example, R_{b3}, R_{b4}, R_{b8} and R_{b9} may each independently be a hydrogen atom, a deuterium atom, a substituted or unsubstituted silyl group, or a substituted or unsubstituted phenyl group.

In Formula 1-6, X_{b1} to X_{b3} may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In some embodiments, in Formula 1-6, X_{b1} to X_{b3} may each independently be combined with an adjacent group to form a ring. For example, X_{b1} to X_{b3} may each independently be a hydrogen atom, a deuterium atom, or a substituted or unsubstituted phenyl group.

In Formula 1-5, b1 and b2 may each independently be an integer of 0 to 3, b3 and b4 may each independently be an integer of 0 to 4, and b5 is an integer of 0 to 5. In Formula 1-5, when b1 to b5 are 0, the nitrogen-containing compound of one or more embodiments may be unsubstituted with R_{b1} to R_{b5}, respectively. In Formula 1-5, a case where b1 and b2 are 3, b3 and b4 are 4, and b5 is 5, and R_{b1} to R_{b5} are all hydrogen atoms, may be the same as a case of Formula 1-5 where b1 to b5 are 0. In Formula 1-5, when b1 to b5 are integers of 2 or more, each of multiple R_{b1} to R_{b5} may be all the same, or at least one selected from among multiple R_{b1} to R_{b5} may be different.

In Formula 1-6, b6 and b7 may each independently be an integer of 0 to 3, b8 and b9 may each independently be an integer of 0 to 4, and m4 to m6 may each independently be an integer of 0 to 5. In Formula 1-6, when b6 to b9, and m1 to m3 are 0, the nitrogen-containing compound of one or more embodiments may be unsubstituted with R_{b6} to R_{b9}, and X_{b1} to X_{b3}, respectively. In Formula 1-6, a case where b6 and b7 are 3, b8 and b9 are 4, m4 to m6 are 5, and R_{b6} to R_{b9} and X_{b1} to X_{b3} are all hydrogen atoms, may be the same as a case of Formula 1-6 where b6 to b9, and m4 to m6 are 0. In Formula 1-6, when b6 to b9, and m4 to m6 are integers of 2 or more, each of multiple R_{b6} to R_{b9} and X_{b1} to X_{b3} may be all the same, or at least one selected from among multiple R_{b6} to R_{b9} and X_{b1} to X_{b3} may be different.

In some embodiments, in Formula 1-5 and Formula 1-6, the same descriptions as those provided in connection with Formula 1 may be applied for Ar₁.

In one or more embodiments, the first compound represented by Formula 1 may be represented by any one selected from among Formula 1-7 to Formula 1-10.

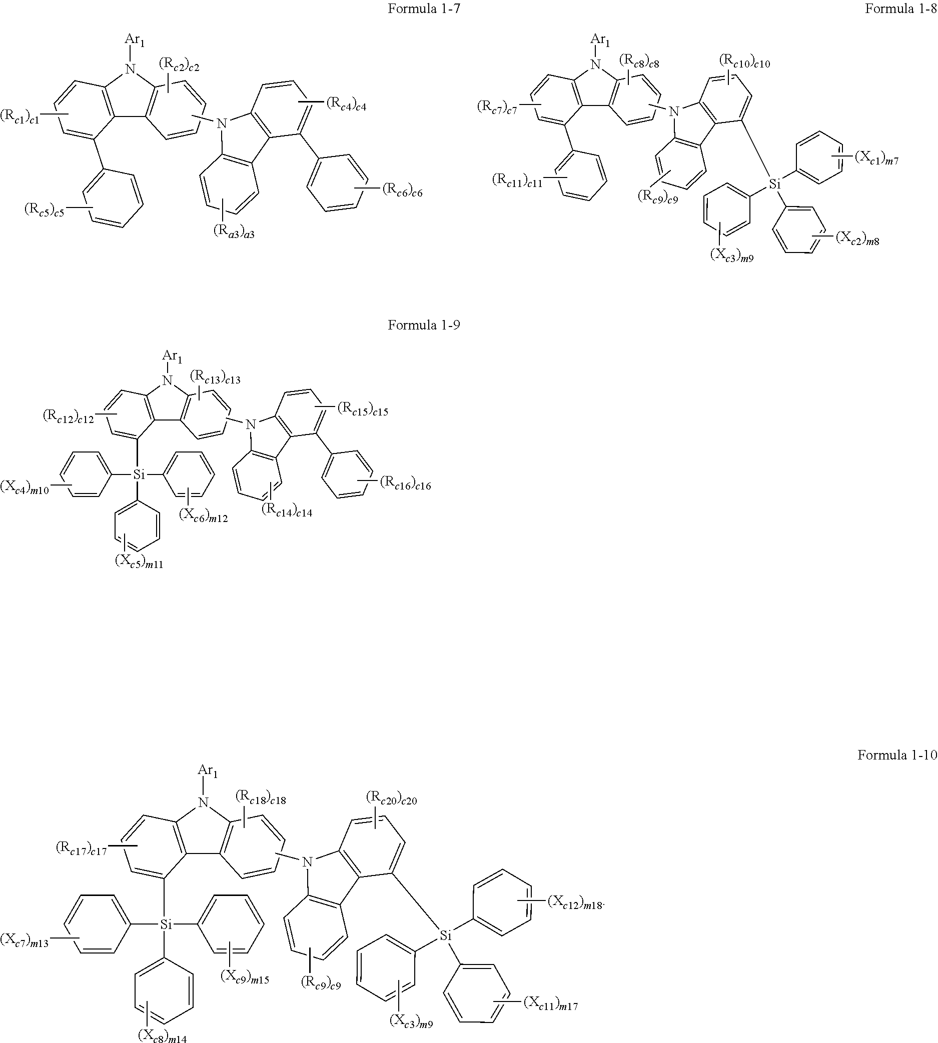

Formula 1-7 to Formula 1-10 represent cases where each of R₄ of Formula 1 and R₁₄ of Formula 2 are a substituted or unsubstituted phenyl group or the substituent represented by Formula 3. For example, Formula 1-7 represents a case where R₄ of Formula 1 and R₁₄ of Formula 2 are substituted or unsubstituted phenyl groups, and Formula 1-8 represents a case where R₄ of Formula 1 is a substituted or unsubstituted phenyl group and R₁₄ of Formula 2 is the substituent represented by Formula 3. Formula 1-9 represents a case where R₄ of Formula 1 is the substituent represented by Formula 3, and R₁₄ of Formula 2 is a substituted or unsubstituted phenyl group, and Formula 1-10 represents a case where R₄ of Formula 1 and R₁₄ of Formula 2 are the substituent represented by Formula 3. Formula 1-7 to Formula 1-10 represent cases where the first substituent is bonded to carbon of position 4 of the first carbazole group and the carbon of position 4 of the second carbazole group.

In Formula 1-7 to Formula 1-10, R_{c1} to R_{c20} may each independently be a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R_{c1} to R_{c20} may each independently be a hydrogen atom, a deuterium atom, or a substituted or unsubstituted phenyl group.

In Formula 1-7 to Formula 1-10, X_{c1} to X_{c12} may each independently be a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In some embodiments, in Formula 1-7 to Formula 1-10, X_{c1} to X_{c12} may each independently be combined with an adjacent group to form a ring. For example, X_{c1} to X_{c12} may each independently be a hydrogen atom, a deuterium atom, or a substituted or unsubstituted phenyl group.

In Formula 1-7, c1, c2 and c4 may each independently be an integer of 0 to 3, c3 is an integer of 0 to 4, and c5 and c6 are integers of 0 to 5. In Formula 1-7, when c1 to c6 are 0, the nitrogen-containing compound of one or more embodiments may be unsubstituted with R_{c1} to R_{c6}, respectively. In Formula 1-7, a case where c1, c2 and c4 are 3, c3 is 4, c5 and c6 are 5, and R_{c1} to R_{c6} are all hydrogen atoms, may be the same as a case of Formula 1-7 where c1 to c6 are 0. In Formula 1-7, when c1 to c6 are integers of 2 or more, each of multiple R_{c1} to R_{c6} may be all the same, or at least one selected from among R_{c1} to R_{c6} may be different.

In Formula 1-8, c7, c8 and c10 may each independently be an integer of 0 to 3, c9 is an integer of 0 to 4, and c11 and m7 to m9 are integers of 0 to 5. In Formula 1-8, when c7 to c11 and m7 to m9 are 0, the nitrogen-containing compound of one or more embodiments may be unsubstituted with R_{c7} to R_{c11} and X_{c1} to X_{c3}, respectively. In Formula 1-8, a case where c7, c8 and c10 are 3, c9 is 4, c11 and m7 to m9 are 5, and R_{c7} to R_{c11} and X_{c1} to X_{c3} are all hydrogen atoms, may be the same as a case of Formula 1-8 where c7 to c11 and m7 to m9 are 0. In Formula 1-8, when c7 to c9 and m7 to m9 are integers of 2 or more, each of multiple R_{c7} to R_{c11} and X_{c1} to X_{c3} may be all the same, or at least one selected from among R_{c7} to R_{c77} and X_{c1} to X_{C3} may be different.

In Formula 1-9, c12, c13 and c15 may each independently be an integer of 0 to 3, c14 is an integer of 0 to 4, and c16 and m10 to m12 are integers of 0 to 5. In Formula 1-9, when c12 to c16 and m10 to m12 are 0, the nitrogen-containing compound of one or more embodiments may be unsubstituted with R_{c12} to R_{c16} and X_{c4} to X_{c6}, respectively. In Formula 1-9, a case where c12, c13 and c15 are 3, c14 is 4, c16 and m10 to m12 are 5, and R_{c12} to R_{c16} and X_{c4} to X_{c6} are all hydrogen atoms, may be the same as a case of Formula 1-9 where c12 to c16 and m10 to m12 are 0. In Formula 1-9, when c12 to c16 and m10 to m12 are integers of 2 or more, each of multiple R_{c12} to R_{c16} and X_{c4} to X_{c6} may be all the same, or at least one selected from among multiple R_{c12} to R_{c16} and X_{c4} to X_{c6} may be different.

In Formula 1-10, c17, c18 and c20 may each independently be an integer of 0 to 3, c19 is an integer of 0 to 4, and m13 to m18 are integers of 0 to 5. In Formula 1-10, when c17 to c20 and m13 to m18 are 0, the nitrogen-containing compound of one or more embodiments may be unsubstituted with R_{c17} to R_{c20} and X_{c7} to X_{c12}, respectively. In Formula 1-10, a case where c17, c18 and c20 are 3, c19 is 4, c16 and m13 to m18 are 5, and R_{c17} to R_{c20} and X_{c7} to X_{c12} are all hydrogen atoms, may be the same as a case of Formula 1-10 where c17 to c20 and m13 to m18 are 0. In Formula 1-10, when c17 to c20 and m13 to m18 are integers of 2 or more, each of multiple R_{c17} to R_{c20} and X_{c7} to X_{c12} may be all the same, or at least one selected from among R_{c17} to R_{c20} and X_{c7} to X_{c12} may be different.

In some embodiments, in Formula 1-7 to Formula 1-10, the same descriptions as those provided in connection with Formula 1 may be applied for Ar₁.

The nitrogen-containing compound of one or more embodiments may be represented by any one selected from among the compounds of Compound Group 1. The light emitting device ED of one or more embodiments may include at least one nitrogen-containing compound selected from among the compounds represented in Compound Group 1 in an emission layer EML.

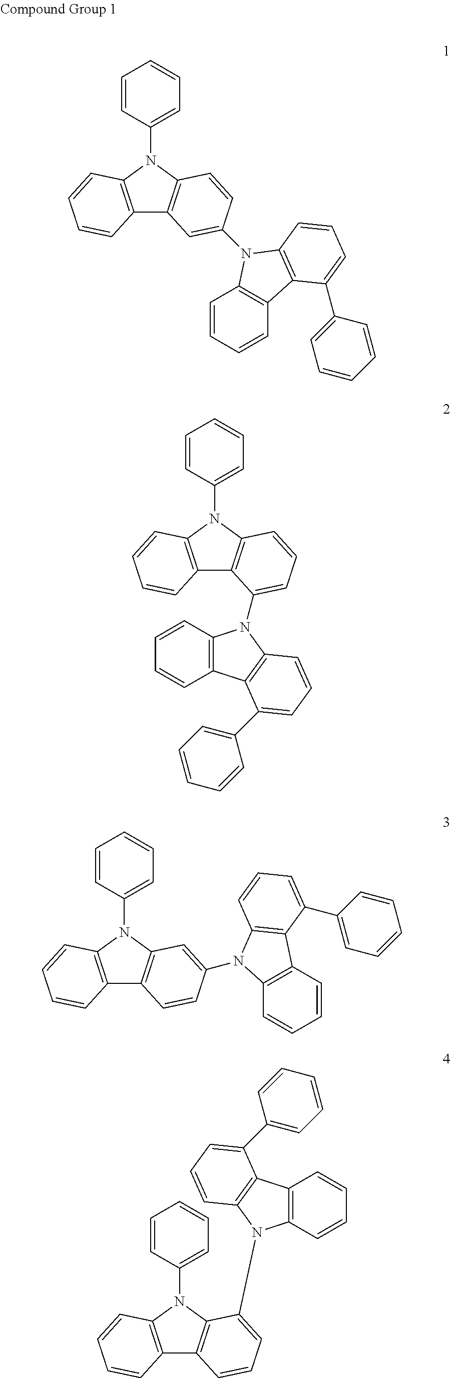

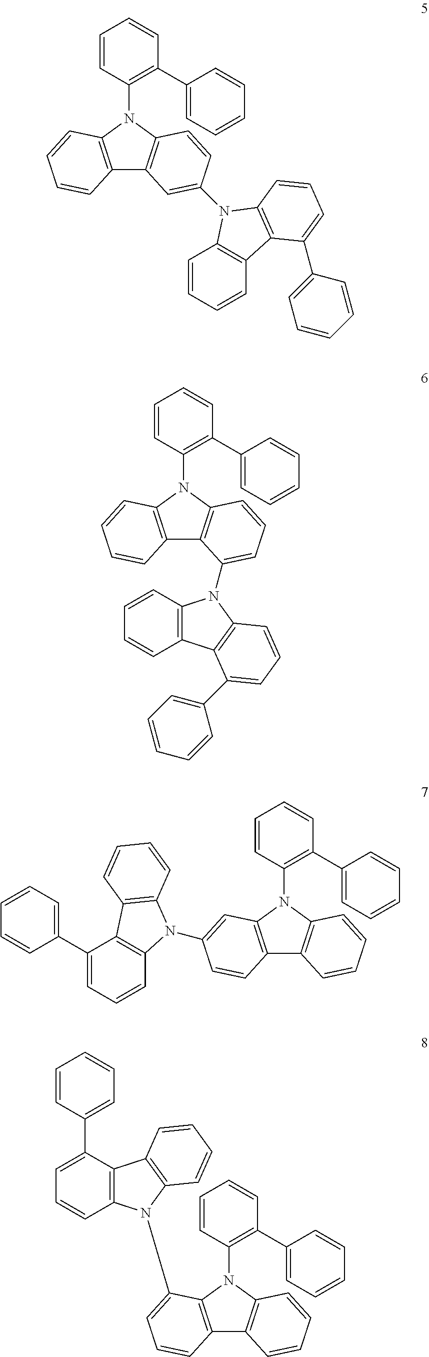 [SMILES: c1ccc(-c2ccccc2-n2c3ccccc3c3cc(-n4c5ccccc5c5c(-c6ccccc6)cccc54)ccc32)cc1 ; c1ccc(-c2ccccc2-n2c3ccccc3c3c(-n4c5ccccc5c5c(-c6ccccc6)cccc54)cccc32)cc1 ; c1ccc(-c2ccccc2-n2c3ccccc3c3ccc(-n4c5ccccc5c5c(-c6ccccc6)cccc54)cc32)cc1 ; c1ccc(-c2ccccc2-n2c3ccccc3c3cccc(-n4c5ccccc5c5c(-c6ccccc6)cccc54)c32)cc1]

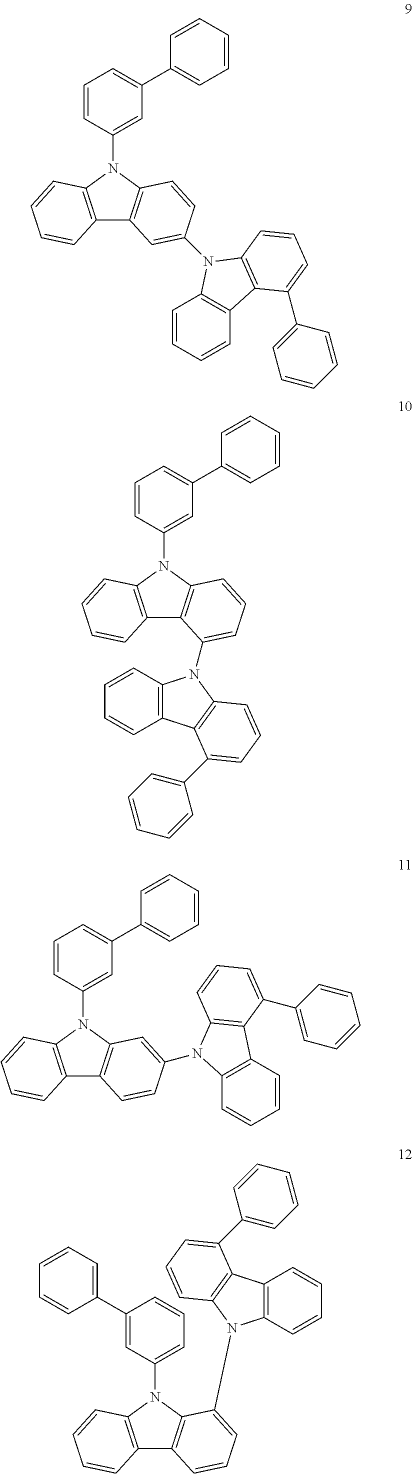

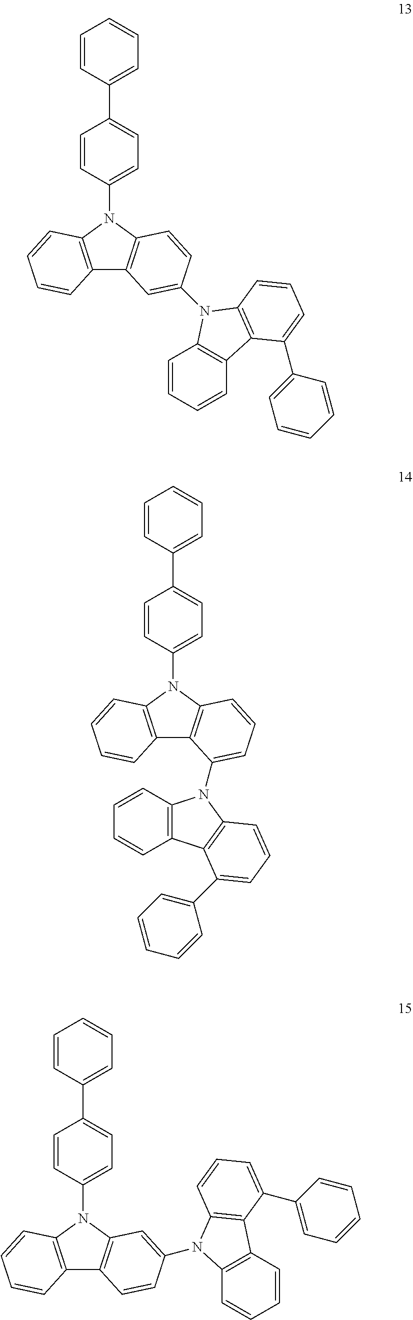 [SMILES: c1ccc(-c2ccc(-n3c4ccccc4c4cc(-n5c6ccccc6c6c(-c7ccccc7)cccc65)ccc43)cc2)cc1 ; c1ccc(-c2ccc(-n3c4ccccc4c4c(-n5c6ccccc6c6c(-c7ccccc7)cccc65)cccc43)cc2)cc1 ; c1ccc(-c2ccc(-n3c4ccccc4c4ccc(-n5c6ccccc6c6c(-c7ccccc7)cccc65)cc43)cc2)cc1]

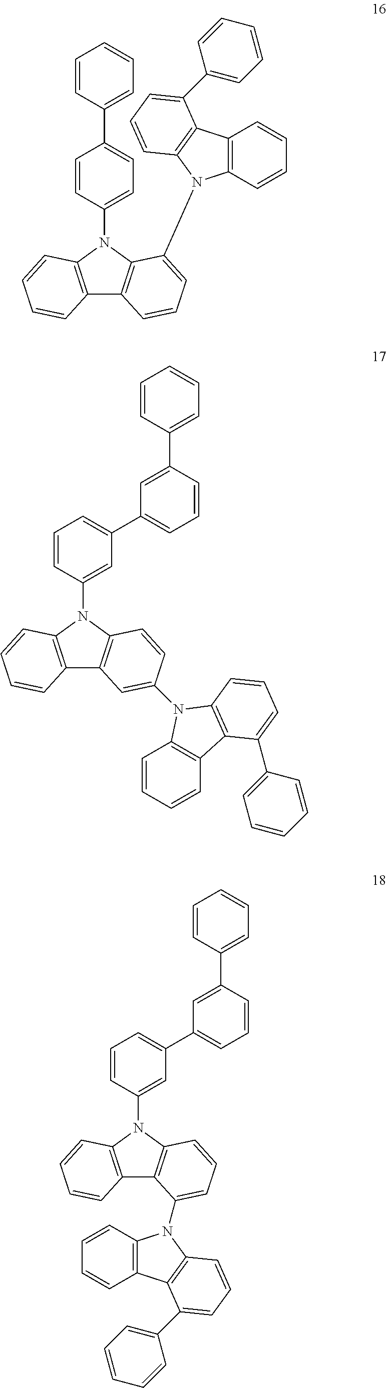 [SMILES: c1ccc(-c2ccc(-n3c4ccccc4c4cccc(-n5c6ccccc6c6c(-c7ccccc7)cccc65)c43)cc2)cc1 ; c1ccc(-c2cccc(-c3cccc(-n4c5ccccc5c5cc(-n6c7ccccc7c7c(-c8ccccc8)cccc76)ccc54)c3)c2)cc1 ; c1ccc(-c2cccc(-c3cccc(-n4c5ccccc5c5c(-n6c7ccccc7c7c(-c8ccccc8)cccc76)cccc54)c3)c2)cc1]

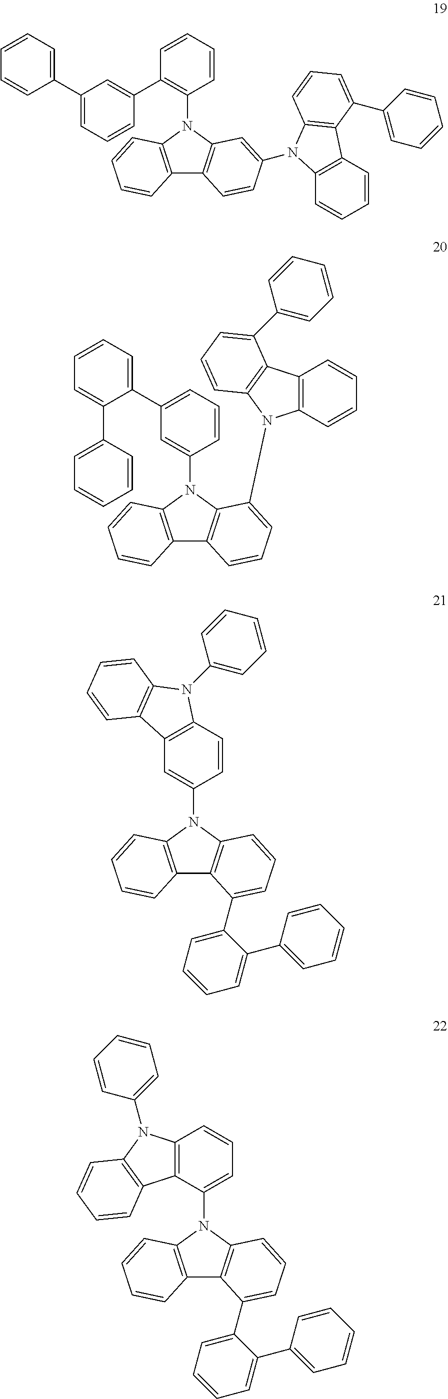

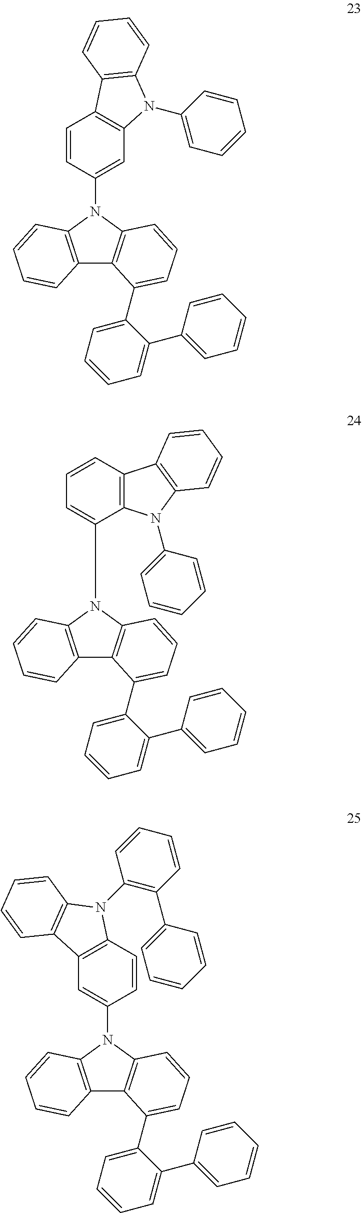 [SMILES: c1ccc(-c2ccccc2-c2cccc3c2c2ccccc2n3-c2ccc3c4ccccc4n(-c4ccccc4)c3c2)cc1 ; c1ccc(-c2ccccc2-c2cccc3c2c2ccccc2n3-c2cccc3c4ccccc4n(-c4ccccc4)c23)cc1 ; c1ccc(-c2ccccc2-c2cccc3c2c2ccccc2n3-c2ccc3c(c2)c2ccccc2n3-c2ccccc2-c2ccccc2)cc1]

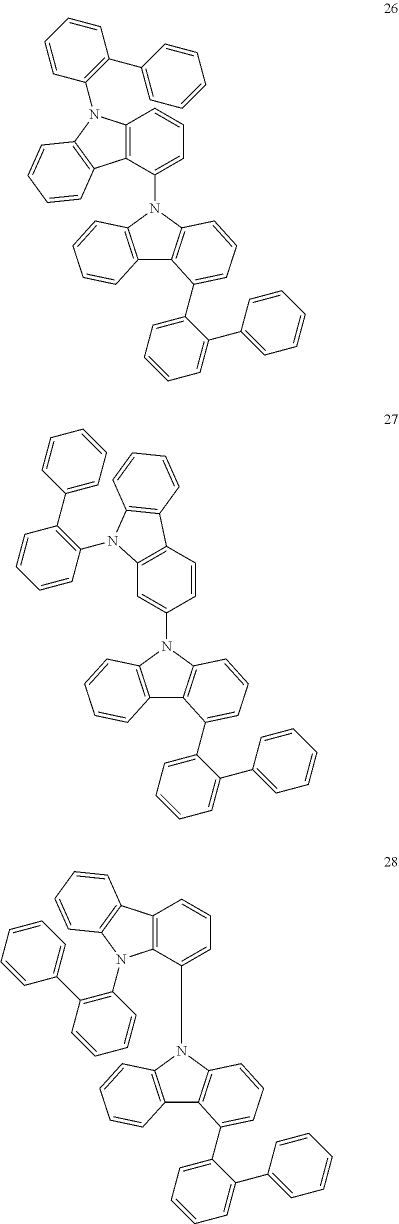 [SMILES: c1ccc(-c2ccccc2-c2cccc3c2c2ccccc2n3-c2cccc3c2c2ccccc2n3-c2ccccc2-c2ccccc2)cc1 ; c1ccc(-c2ccccc2-c2cccc3c2c2ccccc2n3-c2ccc3c4ccccc4n(-c4ccccc4-c4ccccc4)c3c2)cc1 ; c1ccc(-c2ccccc2-c2cccc3c2c2ccccc2n3-c2cccc3c4ccccc4n(-c4ccccc4-c4ccccc4)c23)cc1]

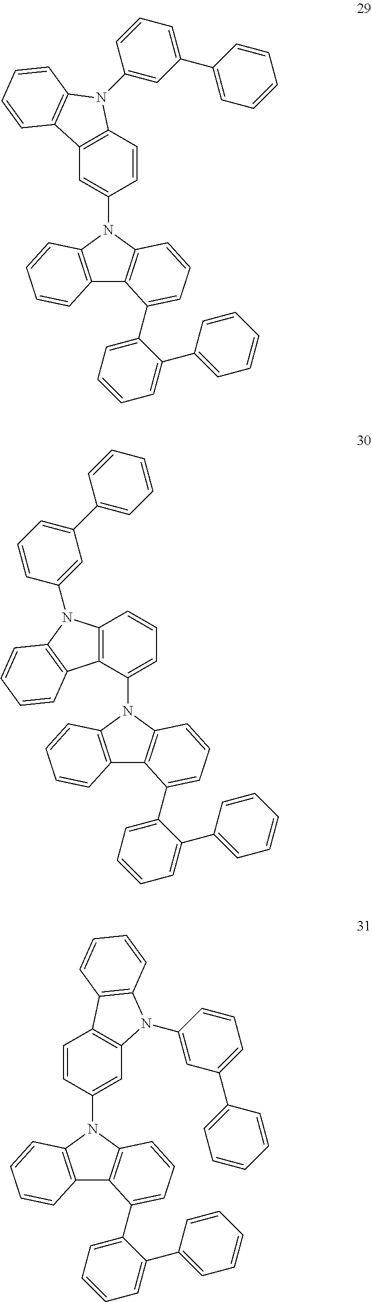 [SMILES: c1ccc(-c2cccc(-n3c4ccccc4c4cc(-n5c6ccccc6c6c(-c7ccccc7-c7ccccc7)cccc65)ccc43)c2)cc1 ; c1ccc(-c2cccc(-n3c4ccccc4c4c(-n5c6ccccc6c6c(-c7ccccc7-c7ccccc7)cccc65)cccc43)c2)cc1 ; c1ccc(-c2cccc(-n3c4ccccc4c4ccc(-n5c6ccccc6c6c(-c7ccccc7-c7ccccc7)cccc65)cc43)c2)cc1]

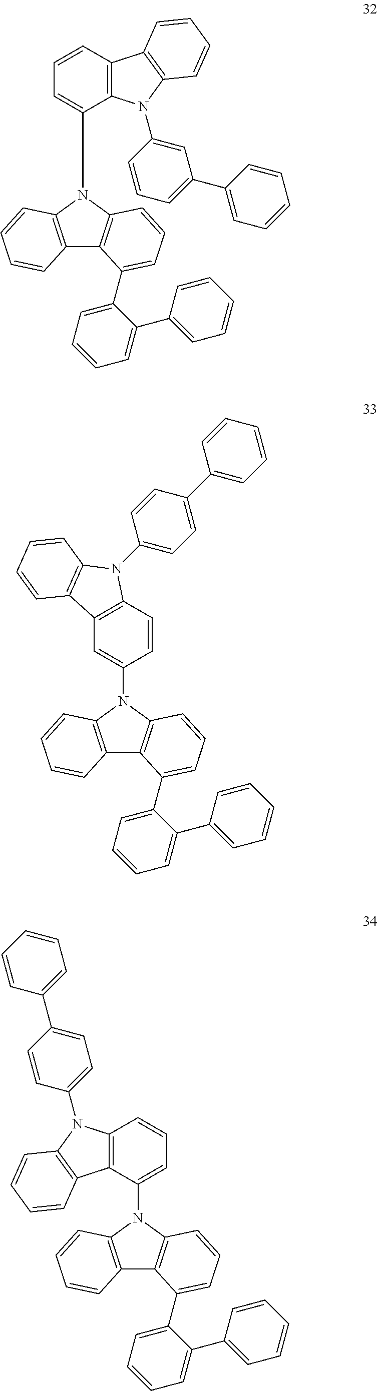 [SMILES: c1ccc(-c2cccc(-n3c4ccccc4c4cccc(-n5c6ccccc6c6c(-c7ccccc7-c7ccccc7)cccc65)c43)c2)cc1 ; c1ccc(-c2ccc(-n3c4ccccc4c4cc(-n5c6ccccc6c6c(-c7ccccc7-c7ccccc7)cccc65)ccc43)cc2)cc1 ; c1ccc(-c2ccc(-n3c4ccccc4c4c(-n5c6ccccc6c6c(-c7ccccc7-c7ccccc7)cccc65)cccc43)cc2)cc1]

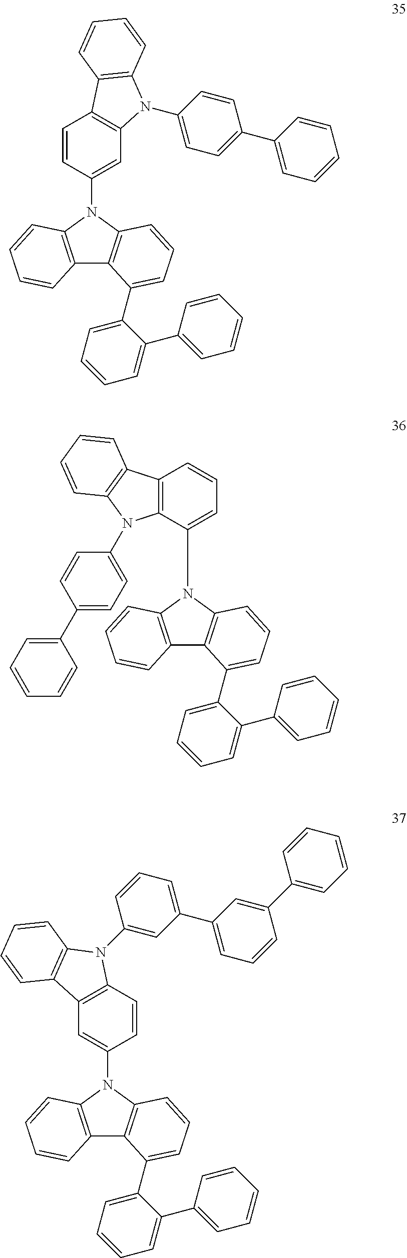 [SMILES: c1ccc(-c2ccc(-n3c4ccccc4c4ccc(-n5c6ccccc6c6c(-c7ccccc7-c7ccccc7)cccc65)cc43)cc2)cc1 ; c1ccc(-c2ccc(-n3c4ccccc4c4cccc(-n5c6ccccc6c6c(-c7ccccc7-c7ccccc7)cccc65)c43)cc2)cc1 ; c1ccc(-c2cccc(-c3cccc(-n4c5ccccc5c5cc(-n6c7ccccc7c7c(-c8ccccc8-c8ccccc8)cccc76)ccc54)c3)c2)cc1]

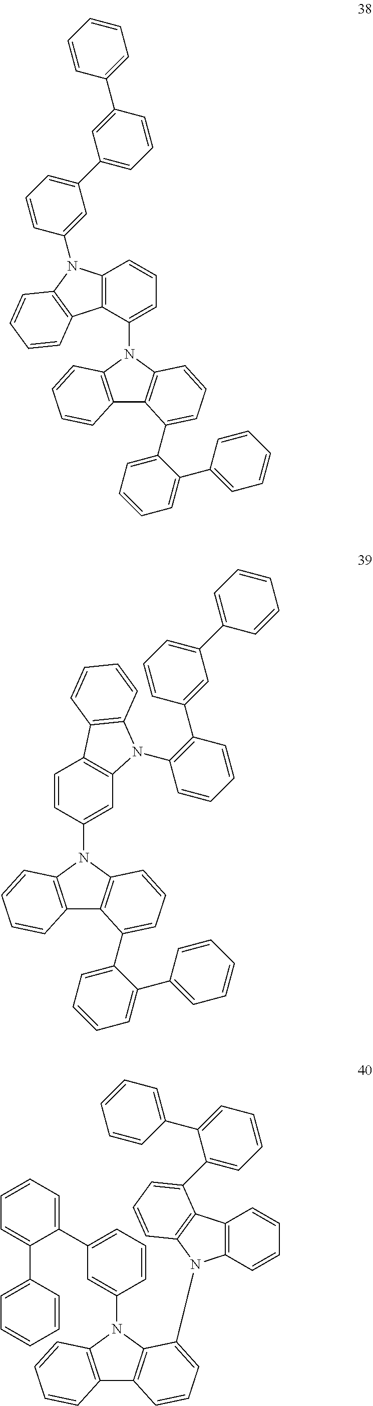 [SMILES: c1ccc(-c2cccc(-c3cccc(-n4c5ccccc5c5c(-n6c7ccccc7c7c(-c8ccccc8-c8ccccc8)cccc76)cccc54)c3)c2)cc1 ; c1ccc(-c2cccc(-c3ccccc3-n3c4ccccc4c4ccc(-n5c6ccccc6c6c(-c7ccccc7-c7ccccc7)cccc65)cc43)c2)cc1 ; c1ccc(-c2ccccc2-c2cccc(-n3c4ccccc4c4cccc(-n5c6ccccc6c6c(-c7ccccc7-c7ccccc7)cccc65)c43)c2)cc1]

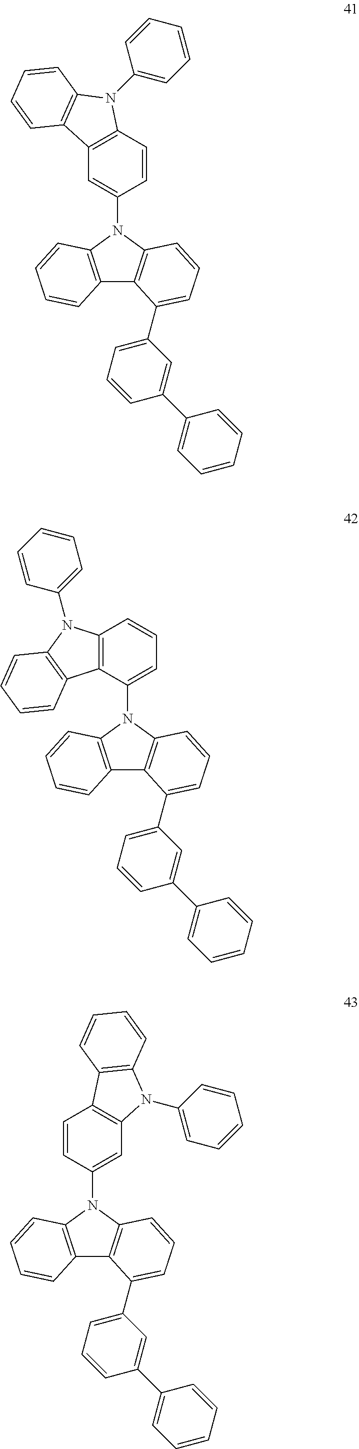 [SMILES: c1ccc(-c2cccc(-c3cccc4c3c3ccccc3n4-c3ccc4c(c3)c3ccccc3n4-c3ccccc3)c2)cc1 ; c1ccc(-c2cccc(-c3cccc4c3c3ccccc3n4-c3cccc4c3c3ccccc3n4-c3ccccc3)c2)cc1 ; c1ccc(-c2cccc(-c3cccc4c3c3ccccc3n4-c3ccc4c5ccccc5n(-c5ccccc5)c4c3)c2)cc1]

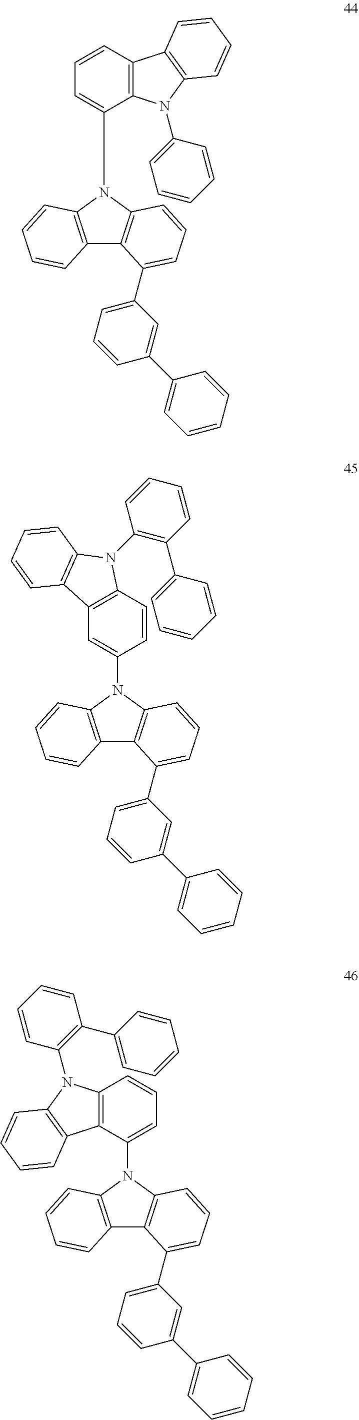 [SMILES: c1ccc(-c2cccc(-c3cccc4c3c3ccccc3n4-c3cccc4c5ccccc5n(-c5ccccc5)c34)c2)cc1 ; c1ccc(-c2cccc(-c3cccc4c3c3ccccc3n4-c3ccc4c(c3)c3ccccc3n4-c3ccccc3-c3ccccc3)c2)cc1 ; c1ccc(-c2cccc(-c3cccc4c3c3ccccc3n4-c3cccc4c3c3ccccc3n4-c3ccccc3-c3ccccc3)c2)cc1]

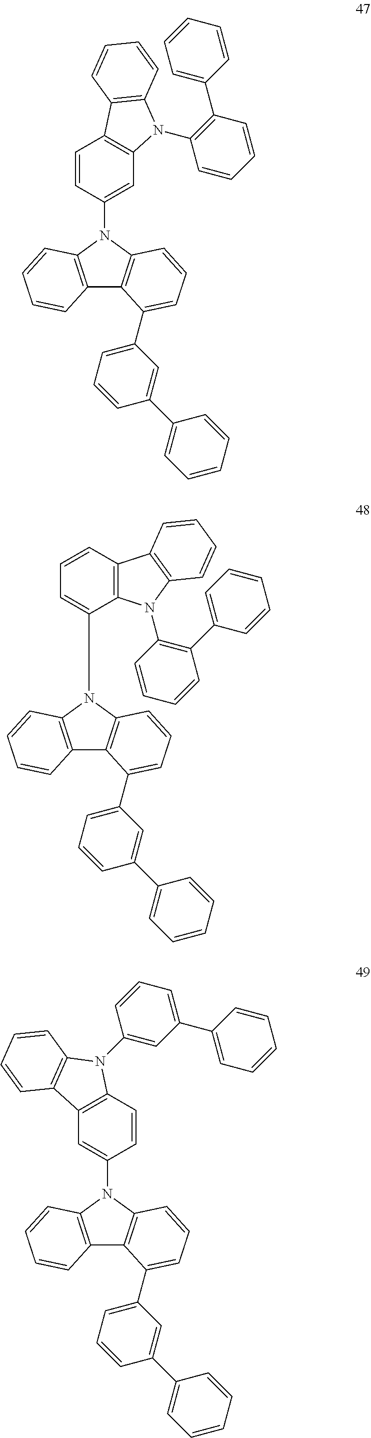

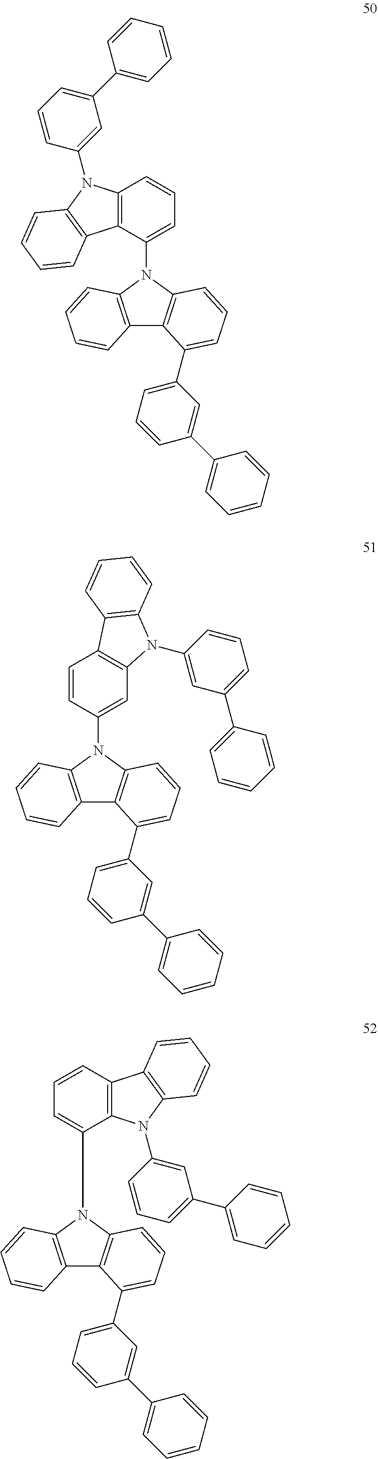

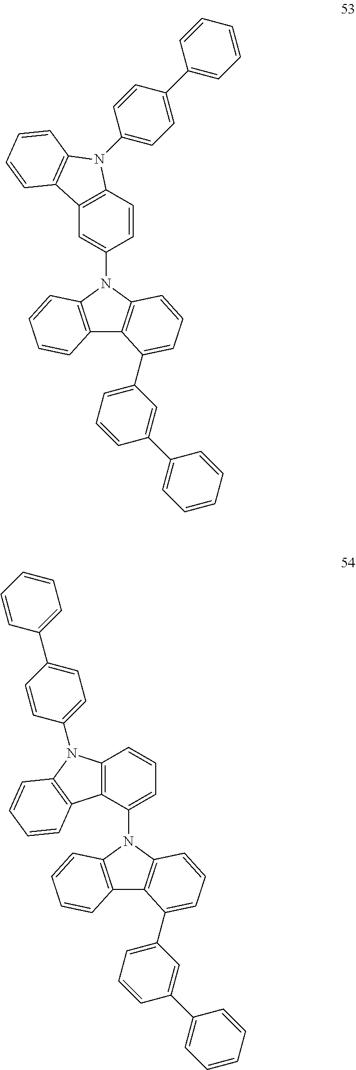 [SMILES: c1ccc(-c2ccc(-n3c4ccccc4c4cc(-n5c6ccccc6c6c(-c7cccc(-c8ccccc8)c7)cccc65)ccc43)cc2)cc1 ; c1ccc(-c2ccc(-n3c4ccccc4c4c(-n5c6ccccc6c6c(-c7cccc(-c8ccccc8)c7)cccc65)cccc43)cc2)cc1]

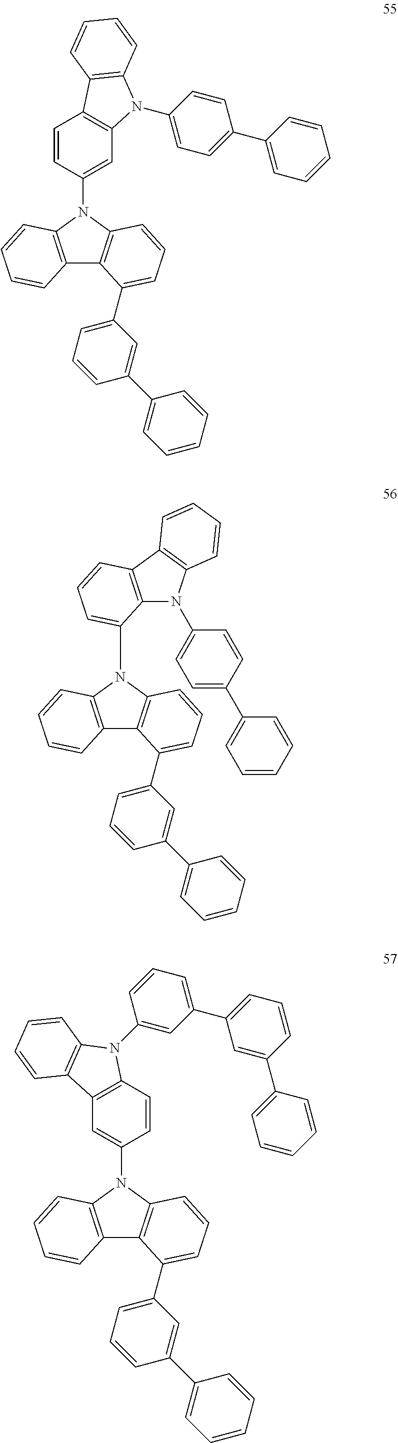 [SMILES: c1ccc(-c2ccc(-n3c4ccccc4c4ccc(-n5c6ccccc6c6c(-c7cccc(-c8ccccc8)c7)cccc65)cc43)cc2)cc1 ; c1ccc(-c2ccc(-n3c4ccccc4c4cccc(-n5c6ccccc6c6c(-c7cccc(-c8ccccc8)c7)cccc65)c43)cc2)cc1 ; c1ccc(-c2cccc(-c3cccc(-n4c5ccccc5c5cc(-n6c7ccccc7c7c(-c8cccc(-c9ccccc9)c8)cccc76)ccc54)c3)c2)cc1]

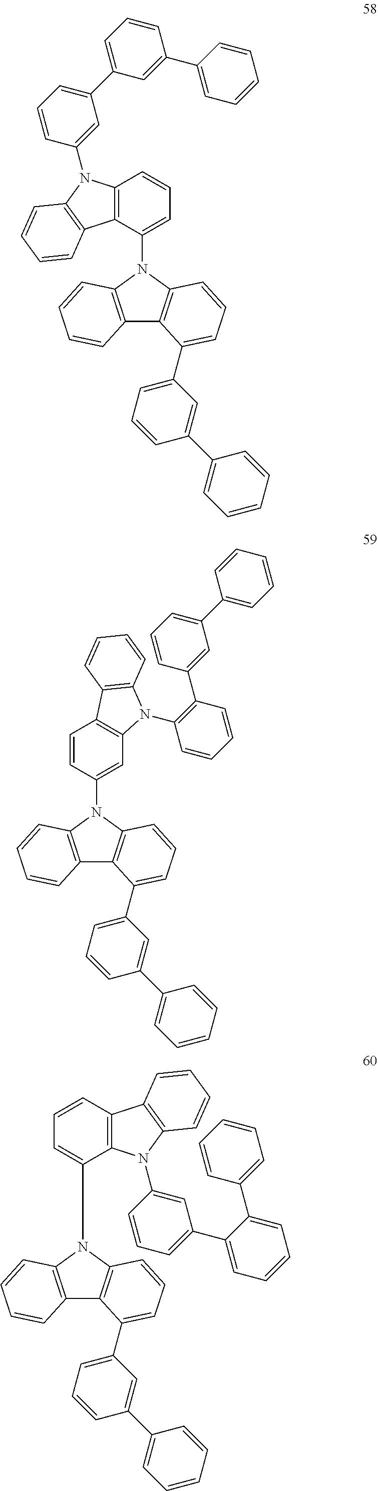

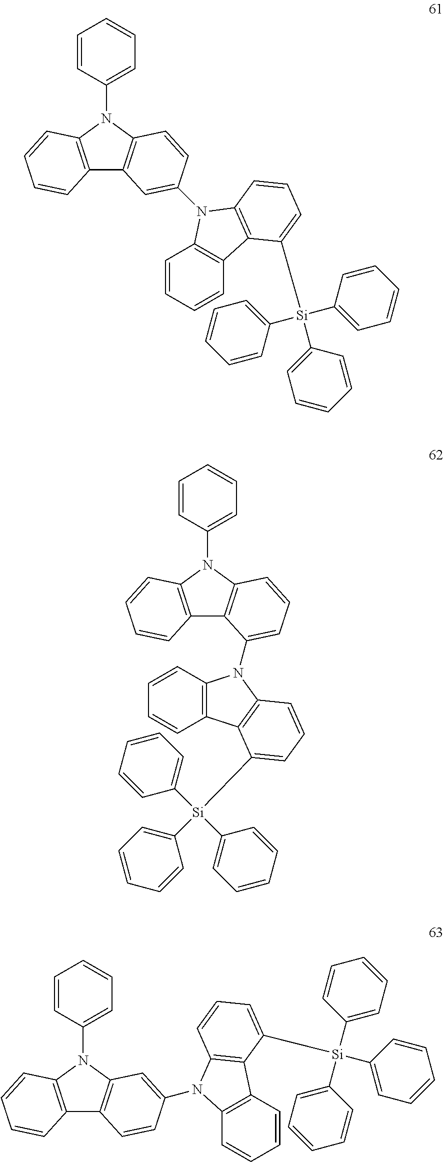 [SMILES: c1ccc(-n2c3ccccc3c3cc(-n4c5ccccc5c5c([Si](c6ccccc6)(c6ccccc6)c6ccccc6)cccc54)ccc32)cc1 ; c1ccc(-n2c3ccccc3c3c(-n4c5ccccc5c5c([Si](c6ccccc6)(c6ccccc6)c6ccccc6)cccc54)cccc32)cc1 ; c1ccc(-n2c3ccccc3c3ccc(-n4c5ccccc5c5c([Si](c6ccccc6)(c6ccccc6)c6ccccc6)cccc54)cc32)cc1]

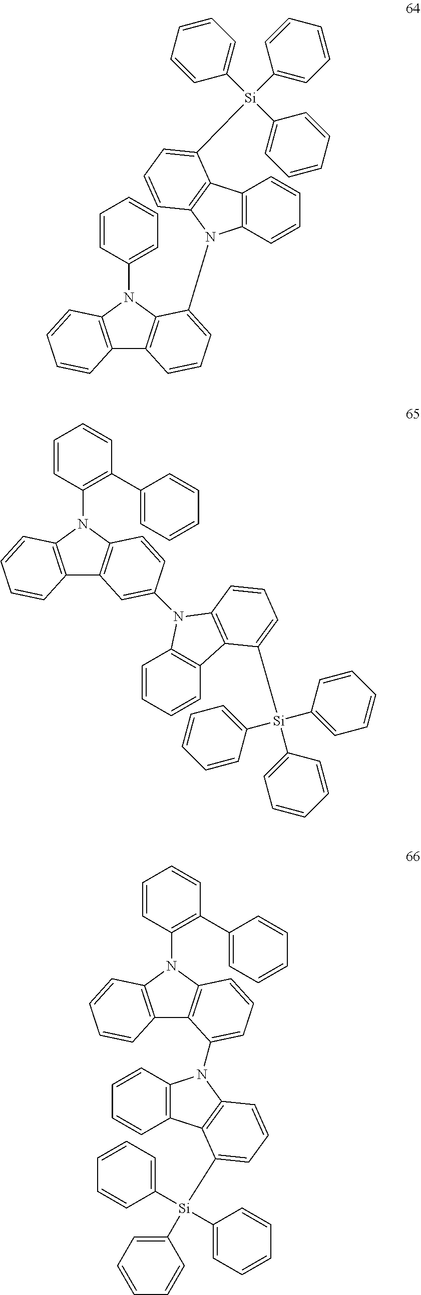 [SMILES: c1ccc(-n2c3ccccc3c3cccc(-n4c5ccccc5c5c([Si](c6ccccc6)(c6ccccc6)c6ccccc6)cccc54)c32)cc1 ; c1ccc(-c2ccccc2-n2c3ccccc3c3cc(-n4c5ccccc5c5c([Si](c6ccccc6)(c6ccccc6)c6ccccc6)cccc54)ccc32)cc1 ; c1ccc(-c2ccccc2-n2c3ccccc3c3c(-n4c5ccccc5c5c([Si](c6ccccc6)(c6ccccc6)c6ccccc6)cccc54)cccc32)cc1]

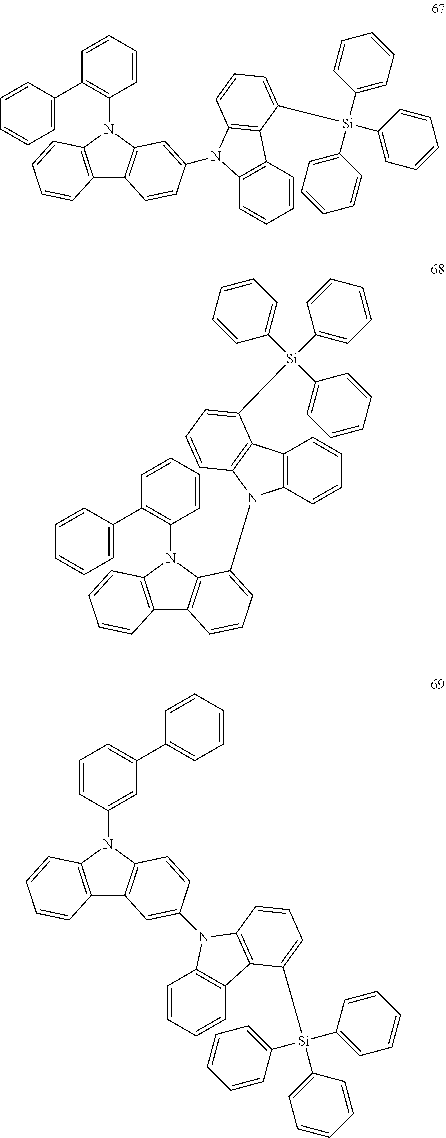 [SMILES: c1ccc(-c2ccccc2-n2c3ccccc3c3ccc(-n4c5ccccc5c5c([Si](c6ccccc6)(c6ccccc6)c6ccccc6)cccc54)cc32)cc1 ; c1ccc(-c2ccccc2-n2c3ccccc3c3cccc(-n4c5ccccc5c5c([Si](c6ccccc6)(c6ccccc6)c6ccccc6)cccc54)c32)cc1 ; c1ccc(-c2cccc(-n3c4ccccc4c4cc(-n5c6ccccc6c6c([Si](c7ccccc7)(c7ccccc7)c7ccccc7)cccc65)ccc43)c2)cc1]

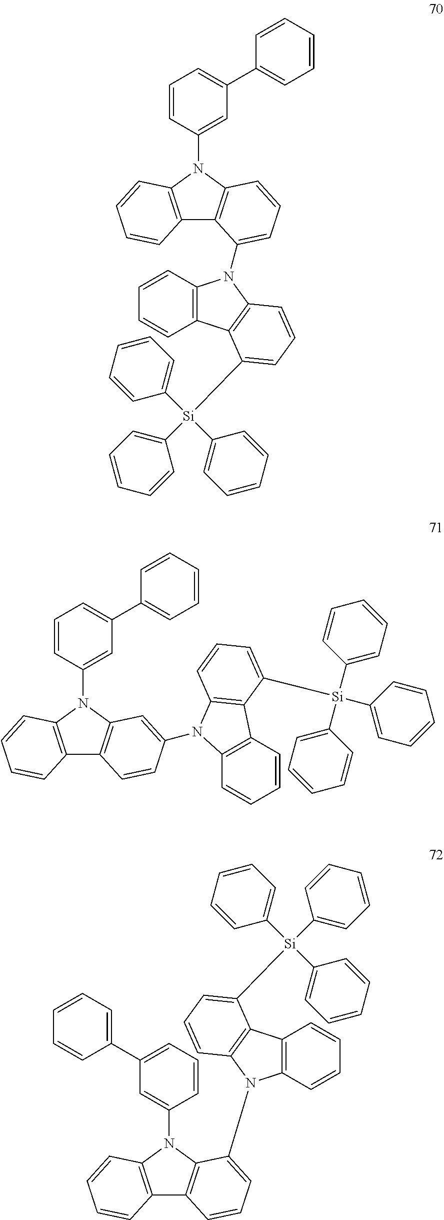 [SMILES: c1ccc(-c2cccc(-n3c4ccccc4c4c(-n5c6ccccc6c6c([Si](c7ccccc7)(c7ccccc7)c7ccccc7)cccc65)cccc43)c2)cc1 ; c1ccc(-c2cccc(-n3c4ccccc4c4ccc(-n5c6ccccc6c6c([Si](c7ccccc7)(c7ccccc7)c7ccccc7)cccc65)cc43)c2)cc1 ; c1ccc(-c2cccc(-n3c4ccccc4c4cccc(-n5c6ccccc6c6c([Si](c7ccccc7)(c7ccccc7)c7ccccc7)cccc65)c43)c2)cc1]

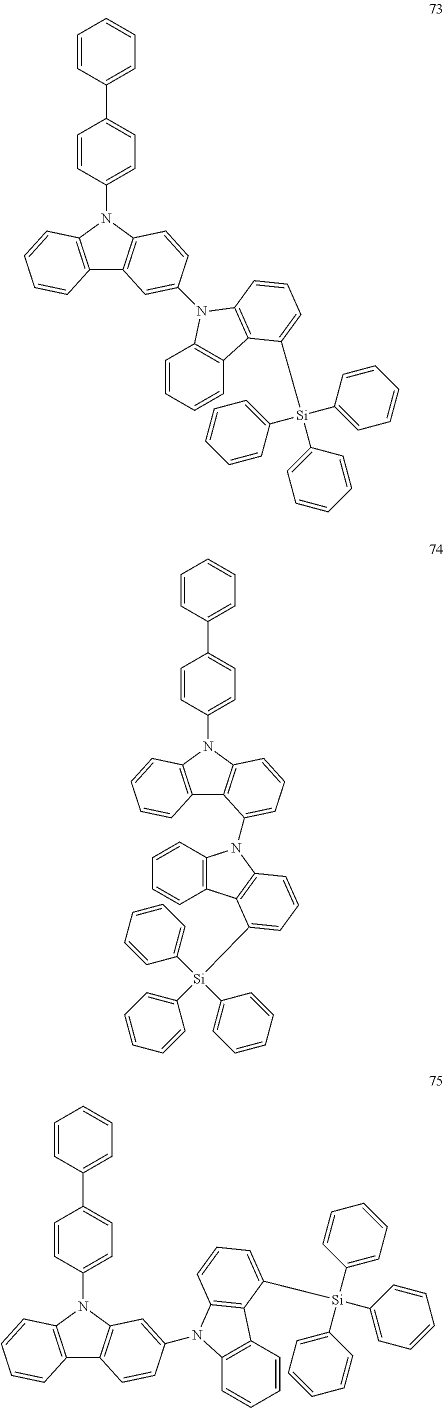 [SMILES: c1ccc(-c2ccc(-n3c4ccccc4c4cc(-n5c6ccccc6c6c([Si](c7ccccc7)(c7ccccc7)c7ccccc7)cccc65)ccc43)cc2)cc1 ; c1ccc(-c2ccc(-n3c4ccccc4c4c(-n5c6ccccc6c6c([Si](c7ccccc7)(c7ccccc7)c7ccccc7)cccc65)cccc43)cc2)cc1 ; c1ccc(-c2ccc(-n3c4ccccc4c4ccc(-n5c6ccccc6c6c([Si](c7ccccc7)(c7ccccc7)c7ccccc7)cccc65)cc43)cc2)cc1]

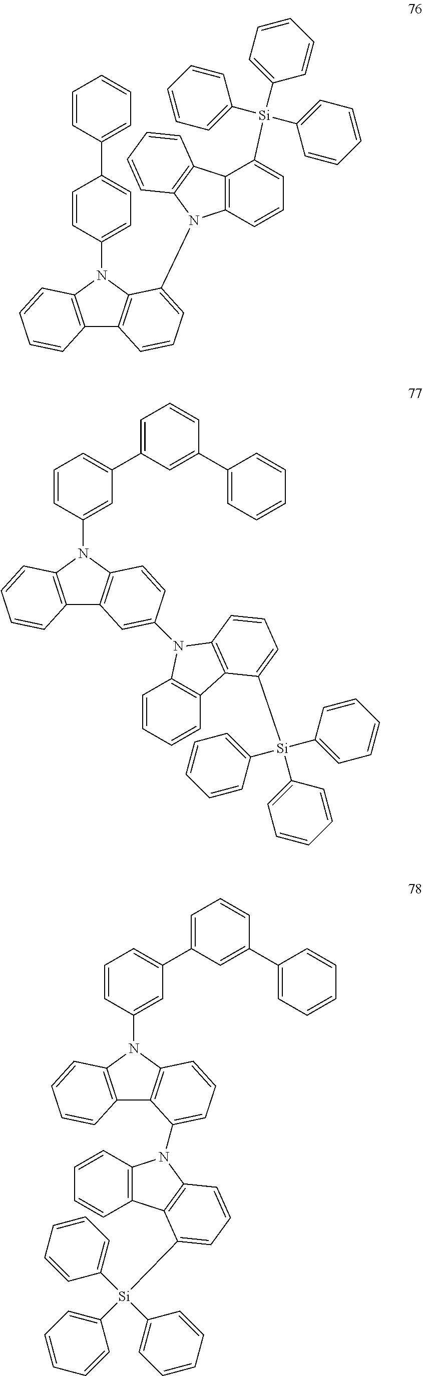 [SMILES: c1ccc(-c2ccc(-n3c4ccccc4c4cccc(-n5c6ccccc6c6c([Si](c7ccccc7)(c7ccccc7)c7ccccc7)cccc65)c43)cc2)cc1 ; c1ccc(-c2cccc(-c3cccc(-n4c5ccccc5c5cc(-n6c7ccccc7c7c([Si](c8ccccc8)(c8ccccc8)c8ccccc8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8ccccc8)cccc76)cccc54)c3)c2)cc1]

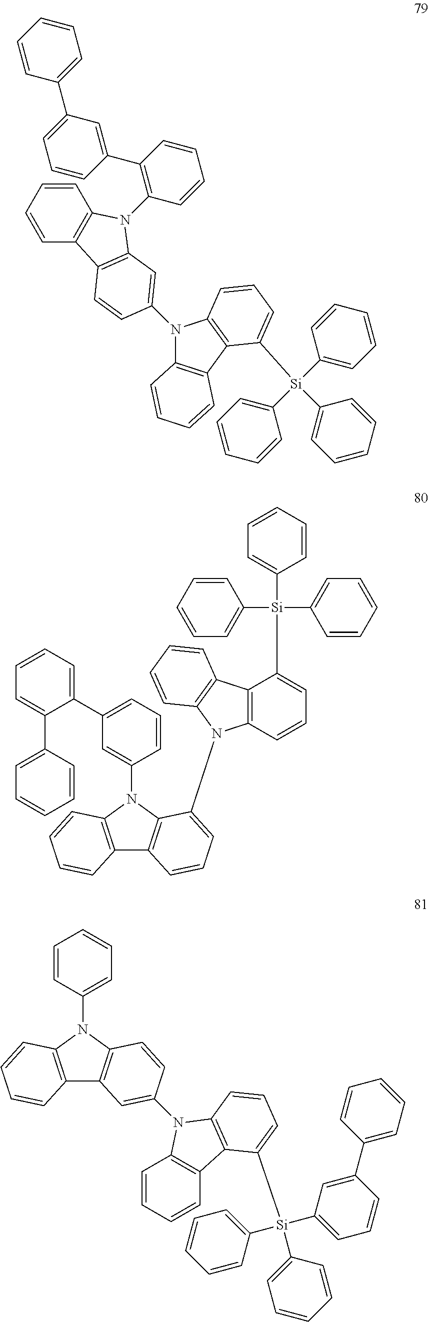 [SMILES: c1ccc(-c2cccc(-c3ccccc3-n3c4ccccc4c4ccc(-n5c6ccccc6c6c([Si](c7ccccc7)(c7ccccc7)c7ccccc7)cccc65)cc43)c2)cc1 ; c1ccc(-c2ccccc2-c2cccc(-n3c4ccccc4c4cccc(-n5c6ccccc6c6c([Si](c7ccccc7)(c7ccccc7)c7ccccc7)cccc65)c43)c2)cc1 ; c1ccc(-c2cccc([Si](c3ccccc3)(c3ccccc3)c3cccc4c3c3ccccc3n4-c3ccc4c(c3)c3ccccc3n4-c3ccccc3)c2)cc1]

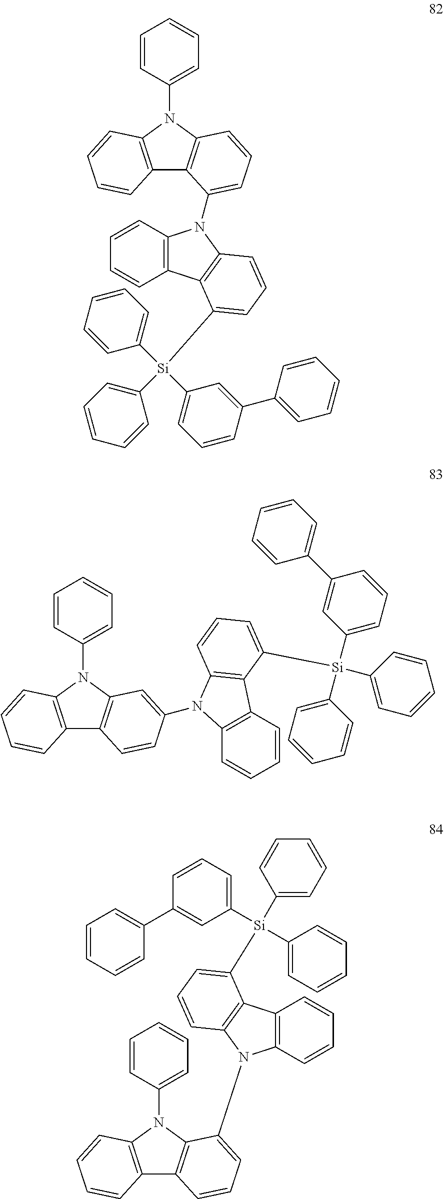 [SMILES: c1ccc(-c2cccc([Si](c3ccccc3)(c3ccccc3)c3cccc4c3c3ccccc3n4-c3cccc4c3c3ccccc3n4-c3ccccc3)c2)cc1 ; c1ccc(-c2cccc([Si](c3ccccc3)(c3ccccc3)c3cccc4c3c3ccccc3n4-c3ccc4c5ccccc5n(-c5ccccc5)c4c3)c2)cc1 ; c1ccc(-c2cccc([Si](c3ccccc3)(c3ccccc3)c3cccc4c3c3ccccc3n4-c3cccc4c5ccccc5n(-c5ccccc5)c34)c2)cc1]

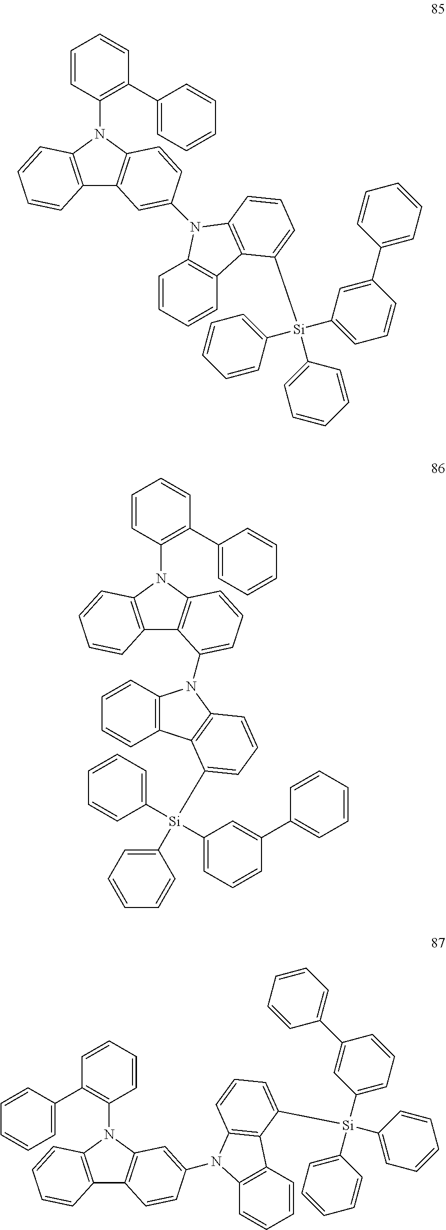 [SMILES: c1ccc(-c2cccc([Si](c3ccccc3)(c3ccccc3)c3cccc4c3c3ccccc3n4-c3ccc4c(c3)c3ccccc3n4-c3ccccc3-c3ccccc3)c2)cc1 ; c1ccc(-c2cccc([Si](c3ccccc3)(c3ccccc3)c3cccc4c3c3ccccc3n4-c3cccc4c3c3ccccc3n4-c3ccccc3-c3ccccc3)c2)cc1 ; c1ccc(-c2cccc([Si](c3ccccc3)(c3ccccc3)c3cccc4c3c3ccccc3n4-c3ccc4c5ccccc5n(-c5ccccc5-c5ccccc5)c4c3)c2)cc1]

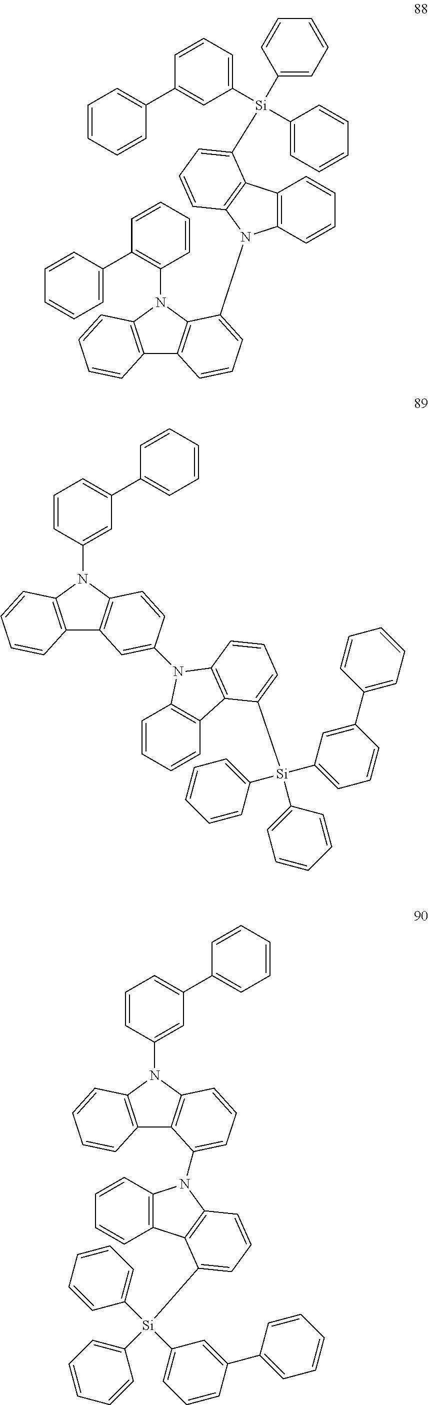 [SMILES: c1ccc(-c2cccc([Si](c3ccccc3)(c3ccccc3)c3cccc4c3c3ccccc3n4-c3cccc4c5ccccc5n(-c5ccccc5-c5ccccc5)c34)c2)cc1 ; c1ccc(-c2cccc(-n3c4ccccc4c4cc(-n5c6ccccc6c6c([Si](c7ccccc7)(c7ccccc7)c7cccc(-c8ccccc8)c7)cccc65)ccc43)c2)cc1 ; c1ccc(-c2cccc(-n3c4ccccc4c4c(-n5c6ccccc6c6c([Si](c7ccccc7)(c7ccccc7)c7cccc(-c8ccccc8)c7)cccc65)cccc43)c2)cc1]

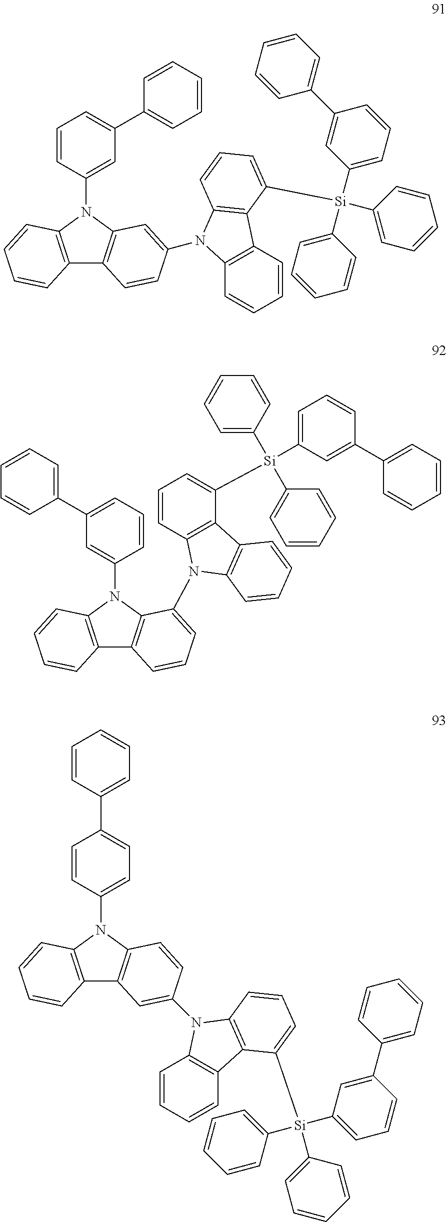 [SMILES: c1ccc(-c2cccc(-n3c4ccccc4c4ccc(-n5c6ccccc6c6c([Si](c7ccccc7)(c7ccccc7)c7cccc(-c8ccccc8)c7)cccc65)cc43)c2)cc1 ; c1ccc(-c2cccc(-n3c4ccccc4c4cccc(-n5c6ccccc6c6c([Si](c7ccccc7)(c7ccccc7)c7cccc(-c8ccccc8)c7)cccc65)c43)c2)cc1 ; c1ccc(-c2ccc(-n3c4ccccc4c4cc(-n5c6ccccc6c6c([Si](c7ccccc7)(c7ccccc7)c7cccc(-c8ccccc8)c7)cccc65)ccc43)cc2)cc1]

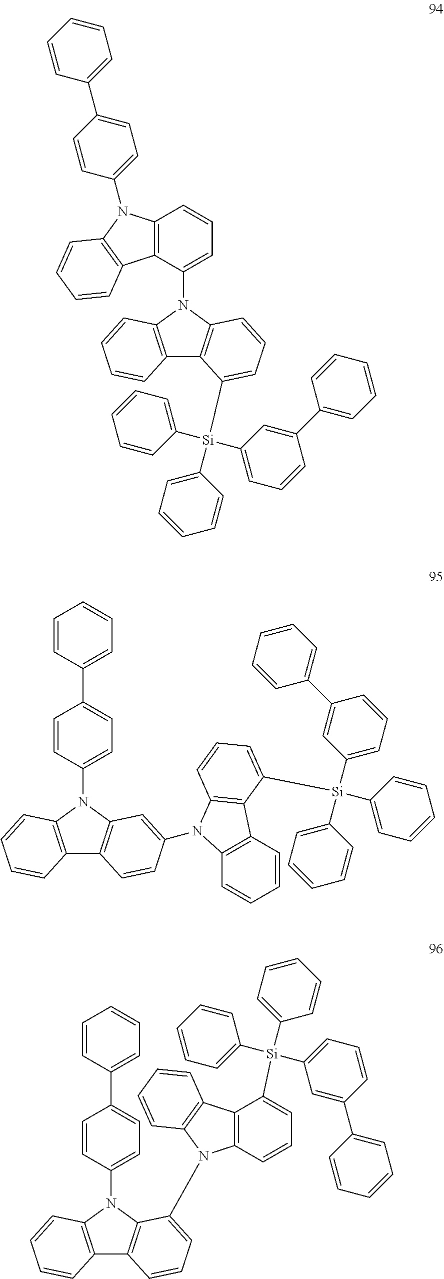 [SMILES: c1ccc(-c2ccc(-n3c4ccccc4c4c(-n5c6ccccc6c6c([Si](c7ccccc7)(c7ccccc7)c7cccc(-c8ccccc8)c7)cccc65)cccc43)cc2)cc1 ; c1ccc(-c2ccc(-n3c4ccccc4c4ccc(-n5c6ccccc6c6c([Si](c7ccccc7)(c7ccccc7)c7cccc(-c8ccccc8)c7)cccc65)cc43)cc2)cc1 ; c1ccc(-c2ccc(-n3c4ccccc4c4cccc(-n5c6ccccc6c6c([Si](c7ccccc7)(c7ccccc7)c7cccc(-c8ccccc8)c7)cccc65)c43)cc2)cc1]

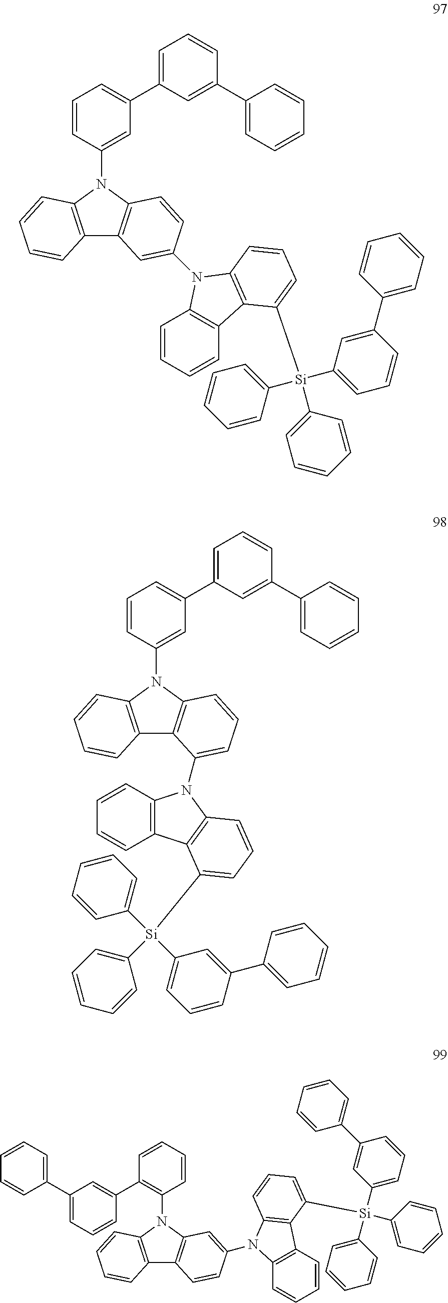 [SMILES: c1ccc(-c2cccc(-c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8cccc(-c9ccccc9)c8)cccc76)cccc54)c3)c2)cc1 ; c1ccc(-c2cccc(-c3ccccc3-n3c4ccccc4c4ccc(-n5c6ccccc6c6c([Si](c7ccccc7)(c7ccccc7)c7cccc(-c8ccccc8)c7)cccc65)cc43)c2)cc1]

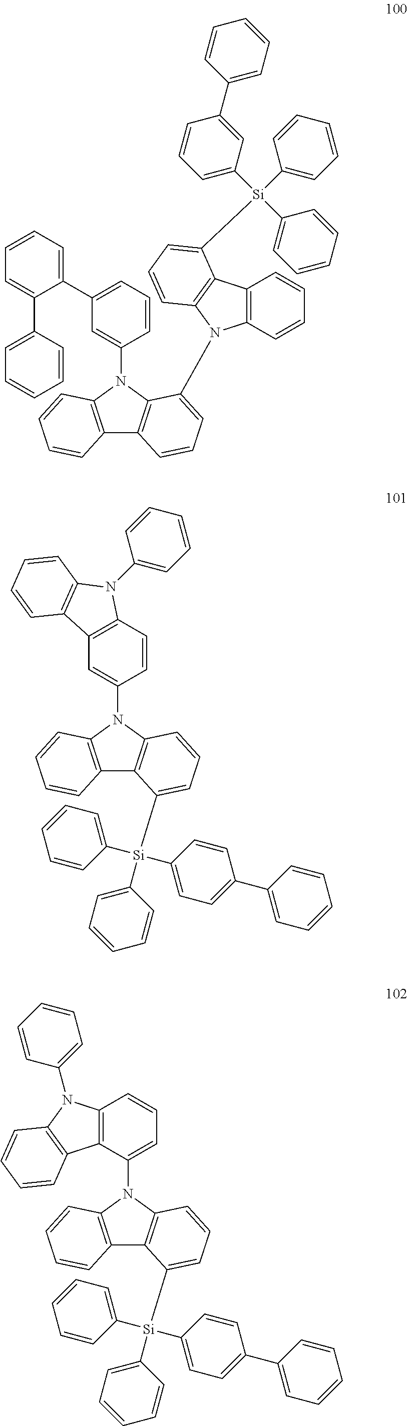 [SMILES: c1ccc(-c2cccc([Si](c3ccccc3)(c3ccccc3)c3cccc4c3c3ccccc3n4-c3cccc4c5ccccc5n(-c5cccc(-c6ccccc6-c6ccccc6)c5)c34)c2)cc1 ; c1ccc(-c2ccc([Si](c3ccccc3)(c3ccccc3)c3cccc4c3c3ccccc3n4-c3ccc4c(c3)c3ccccc3n4-c3ccccc3)cc2)cc1 ; c1ccc(-c2ccc([Si](c3ccccc3)(c3ccccc3)c3cccc4c3c3ccccc3n4-c3cccc4c3c3ccccc3n4-c3ccccc3)cc2)cc1]

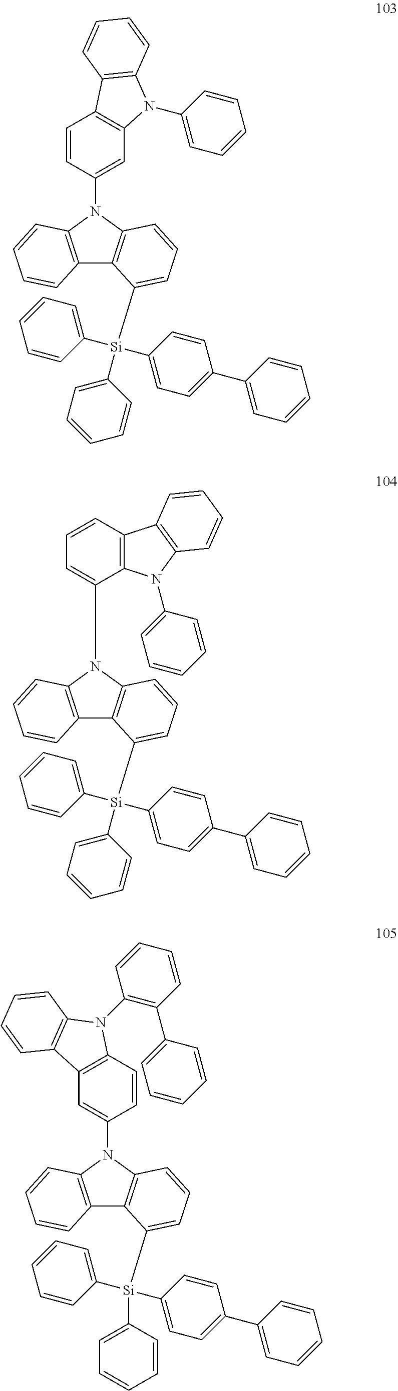 [SMILES: c1ccc(-c2ccc([Si](c3ccccc3)(c3ccccc3)c3cccc4c3c3ccccc3n4-c3ccc4c5ccccc5n(-c5ccccc5)c4c3)cc2)cc1 ; c1ccc(-c2ccc([Si](c3ccccc3)(c3ccccc3)c3cccc4c3c3ccccc3n4-c3cccc4c5ccccc5n(-c5ccccc5)c34)cc2)cc1 ; c1ccc(-c2ccc([Si](c3ccccc3)(c3ccccc3)c3cccc4c3c3ccccc3n4-c3ccc4c(c3)c3ccccc3n4-c3ccccc3-c3ccccc3)cc2)cc1]

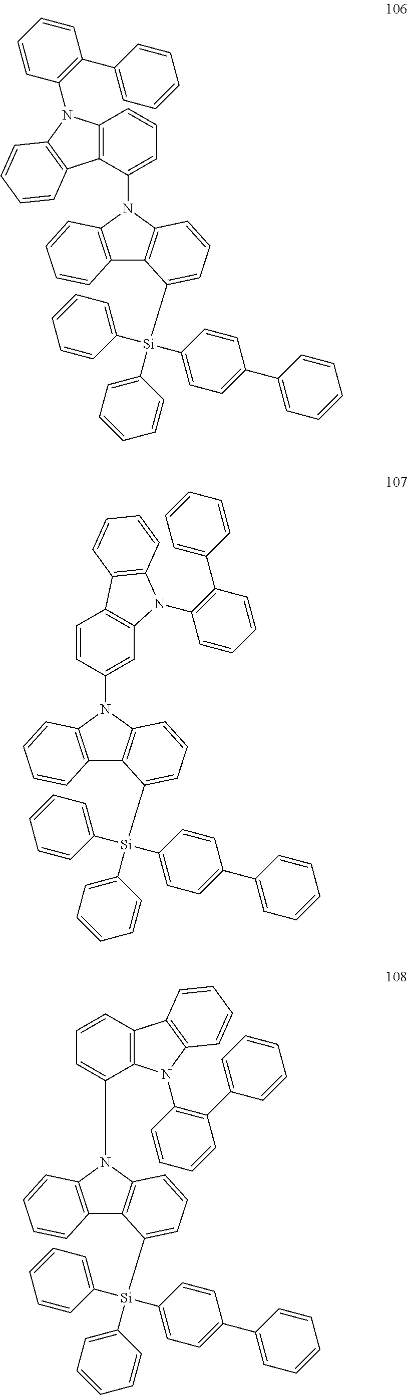 [SMILES: c1ccc(-c2ccc([Si](c3ccccc3)(c3ccccc3)c3cccc4c3c3ccccc3n4-c3cccc4c3c3ccccc3n4-c3ccccc3-c3ccccc3)cc2)cc1 ; c1ccc(-c2ccc([Si](c3ccccc3)(c3ccccc3)c3cccc4c3c3ccccc3n4-c3ccc4c5ccccc5n(-c5ccccc5-c5ccccc5)c4c3)cc2)cc1 ; c1ccc(-c2ccc([Si](c3ccccc3)(c3ccccc3)c3cccc4c3c3ccccc3n4-c3cccc4c5ccccc5n(-c5ccccc5-c5ccccc5)c34)cc2)cc1]

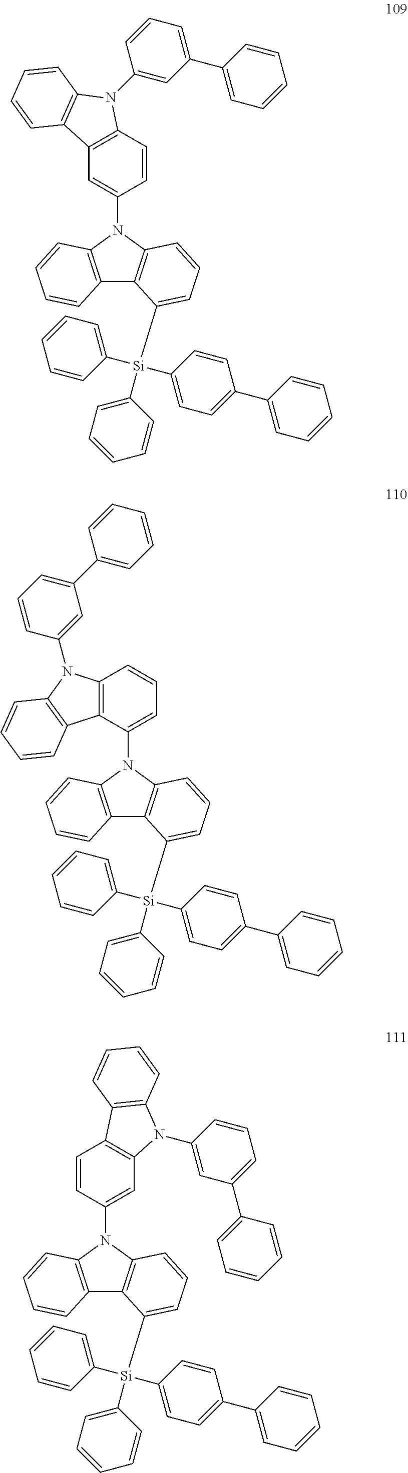

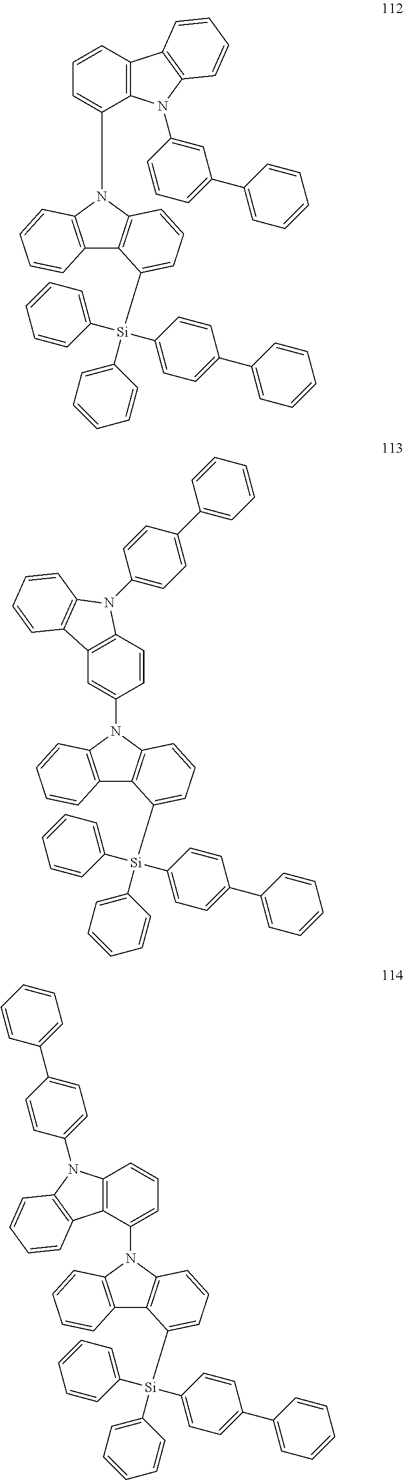 [SMILES: c1ccc(-c2ccc([Si](c3ccccc3)(c3ccccc3)c3cccc4c3c3ccccc3n4-c3cccc4c5ccccc5n(-c5cccc(-c6ccccc6)c5)c34)cc2)cc1 ; c1ccc(-c2ccc(-n3c4ccccc4c4cc(-n5c6ccccc6c6c([Si](c7ccccc7)(c7ccccc7)c7ccc(-c8ccccc8)cc7)cccc65)ccc43)cc2)cc1 ; c1ccc(-c2ccc(-n3c4ccccc4c4c(-n5c6ccccc6c6c([Si](c7ccccc7)(c7ccccc7)c7ccc(-c8ccccc8)cc7)cccc65)cccc43)cc2)cc1]

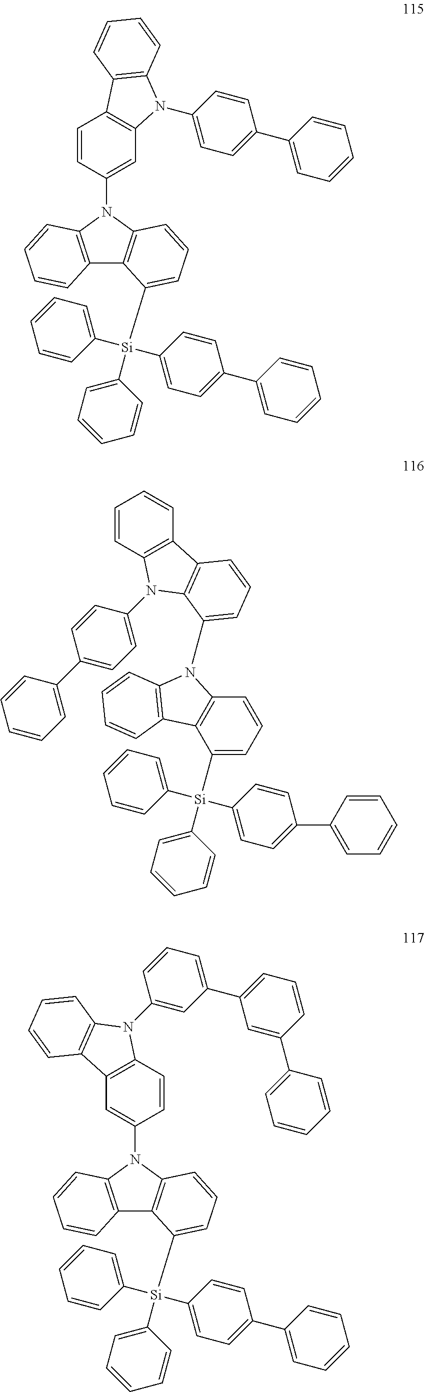 [SMILES: c1ccc(-c2ccc(-n3c4ccccc4c4ccc(-n5c6ccccc6c6c([Si](c7ccccc7)(c7ccccc7)c7ccc(-c8ccccc8)cc7)cccc65)cc43)cc2)cc1 ; c1ccc(-c2ccc(-n3c4ccccc4c4cccc(-n5c6ccccc6c6c([Si](c7ccccc7)(c7ccccc7)c7ccc(-c8ccccc8)cc7)cccc65)c43)cc2)cc1 ; c1ccc(-c2ccc([Si](c3ccccc3)(c3ccccc3)c3cccc4c3c3ccccc3n4-c3ccc4c(c3)c3ccccc3n4-c3cccc(-c4cccc(-c5ccccc5)c4)c3)cc2)cc1]

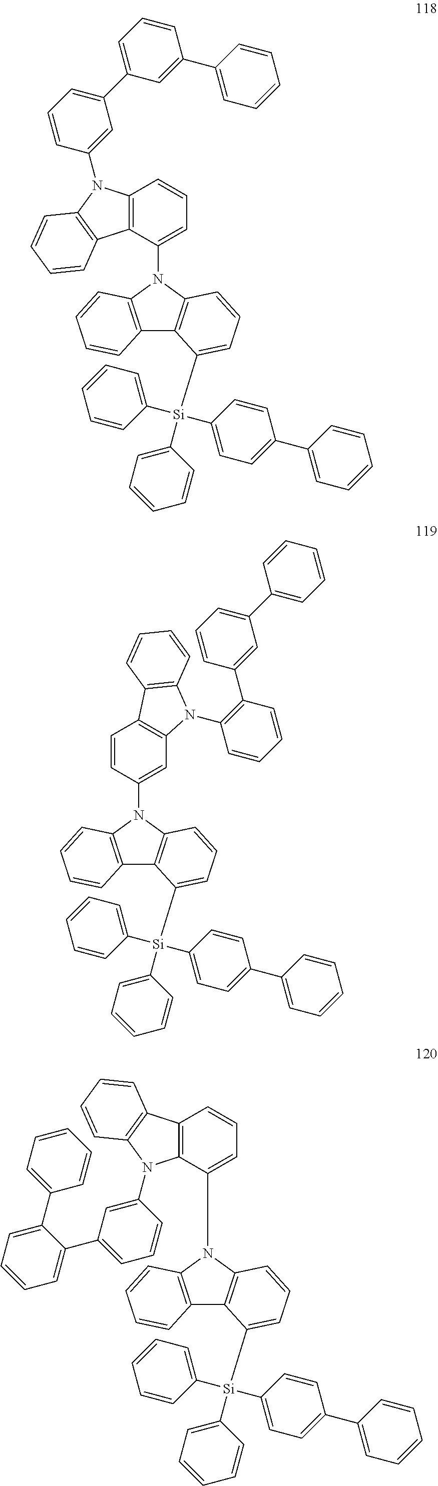

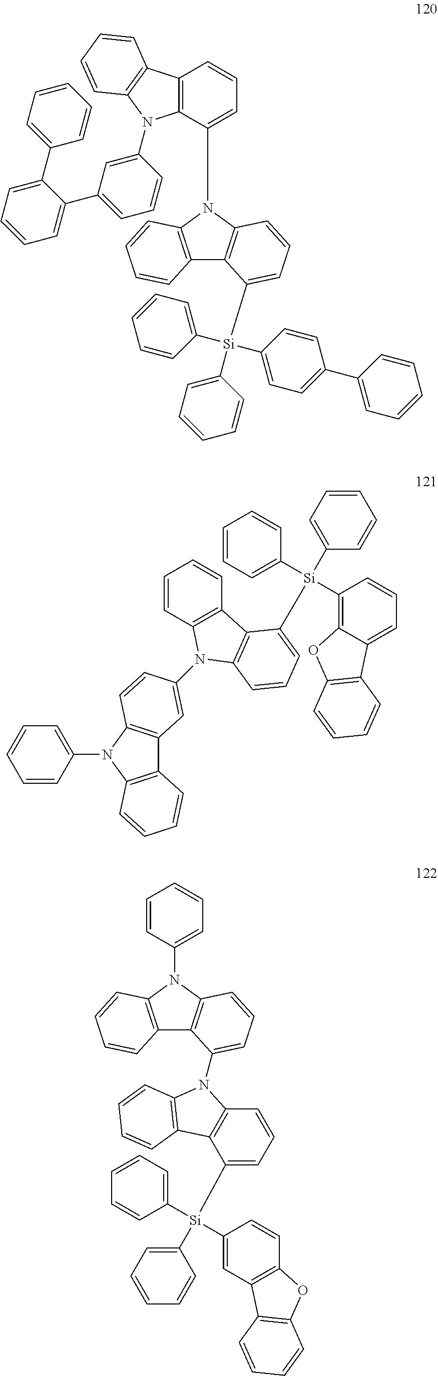 [SMILES: c1ccc(-c2ccc([Si](c3ccccc3)(c3ccccc3)c3cccc4c3c3ccccc3n4-c3cccc4c5ccccc5n(-c5cccc(-c6ccccc6-c6ccccc6)c5)c34)cc2)cc1 ; c1ccc(-n2c3ccccc3c3cc(-n4c5ccccc5c5c([Si](c6ccccc6)(c6ccccc6)c6cccc7c6oc6ccccc67)cccc54)ccc32)cc1 ; c1ccc(-n2c3ccccc3c3c(-n4c5ccccc5c5c([Si](c6ccccc6)(c6ccccc6)c6ccc7oc8ccccc8c7c6)cccc54)cccc32)cc1]

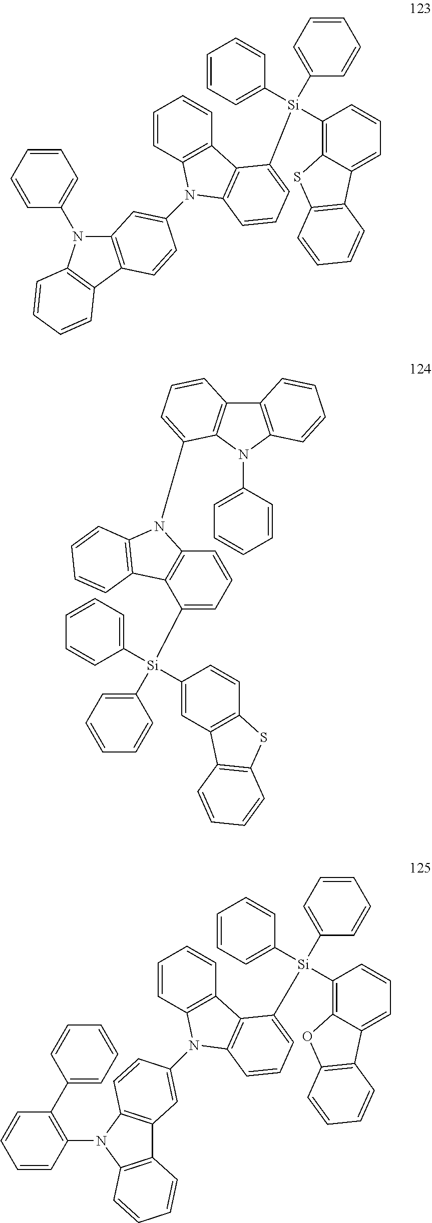 [SMILES: c1ccc(-n2c3ccccc3c3ccc(-n4c5ccccc5c5c([Si](c6ccccc6)(c6ccccc6)c6cccc7c6sc6ccccc67)cccc54)cc32)cc1 ; c1ccc(-n2c3ccccc3c3cccc(-n4c5ccccc5c5c([Si](c6ccccc6)(c6ccccc6)c6ccc7sc8ccccc8c7c6)cccc54)c32)cc1 ; c1ccc(-c2ccccc2-n2c3ccccc3c3cc(-n4c5ccccc5c5c([Si](c6ccccc6)(c6ccccc6)c6cccc7c6oc6ccccc67)cccc54)ccc32)cc1]

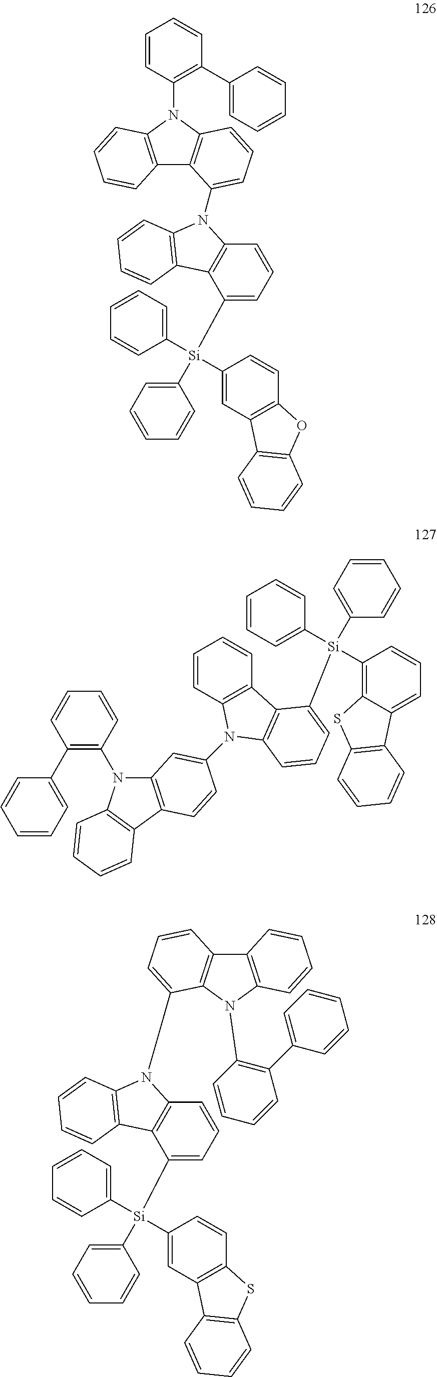 [SMILES: c1ccc(-c2ccccc2-n2c3ccccc3c3c(-n4c5ccccc5c5c([Si](c6ccccc6)(c6ccccc6)c6ccc7oc8ccccc8c7c6)cccc54)cccc32)cc1 ; c1ccc(-c2ccccc2-n2c3ccccc3c3ccc(-n4c5ccccc5c5c([Si](c6ccccc6)(c6ccccc6)c6cccc7c6sc6ccccc67)cccc54)cc32)cc1 ; c1ccc(-c2ccccc2-n2c3ccccc3c3cccc(-n4c5ccccc5c5c([Si](c6ccccc6)(c6ccccc6)c6ccc7sc8ccccc8c7c6)cccc54)c32)cc1]

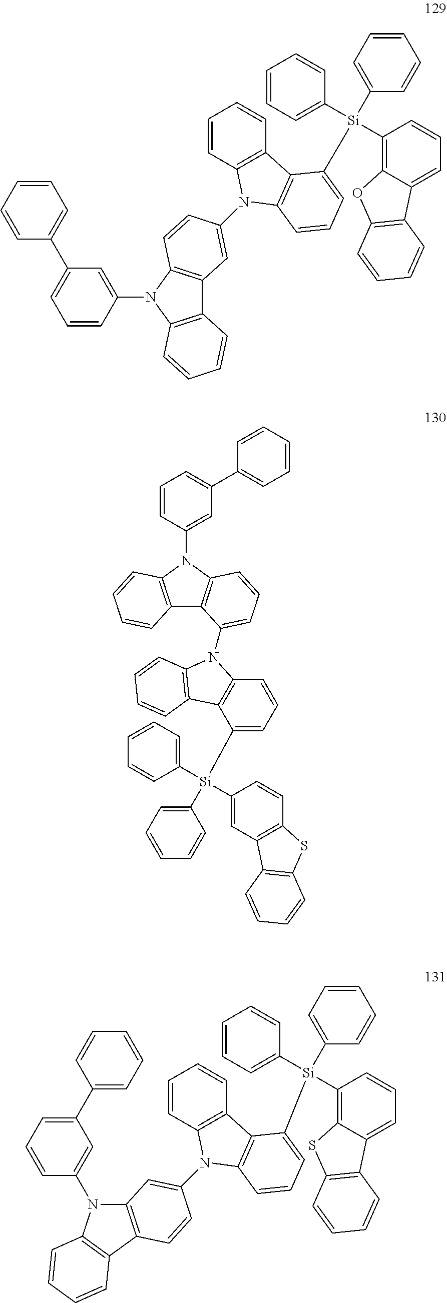 [SMILES: c1ccc(-c2cccc(-n3c4ccccc4c4cc(-n5c6ccccc6c6c([Si](c7ccccc7)(c7ccccc7)c7cccc8c7oc7ccccc78)cccc65)ccc43)c2)cc1 ; c1ccc(-c2cccc(-n3c4ccccc4c4c(-n5c6ccccc6c6c([Si](c7ccccc7)(c7ccccc7)c7ccc8sc9ccccc9c8c7)cccc65)cccc43)c2)cc1 ; c1ccc(-c2cccc(-n3c4ccccc4c4ccc(-n5c6ccccc6c6c([Si](c7ccccc7)(c7ccccc7)c7cccc8c7sc7ccccc78)cccc65)cc43)c2)cc1]

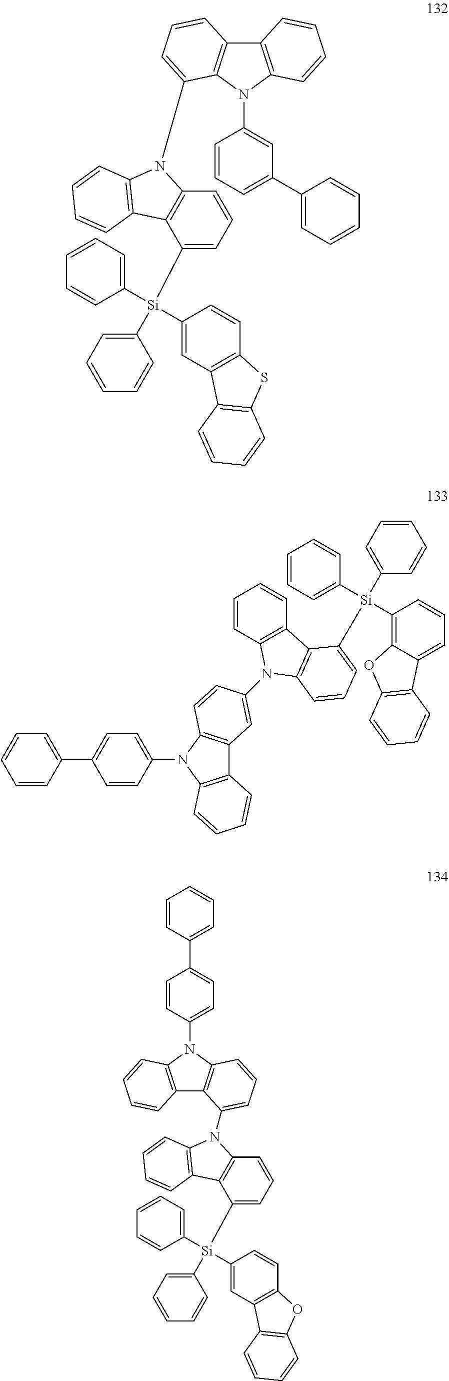 [SMILES: c1ccc(-c2cccc(-n3c4ccccc4c4cccc(-n5c6ccccc6c6c([Si](c7ccccc7)(c7ccccc7)c7ccc8sc9ccccc9c8c7)cccc65)c43)c2)cc1 ; c1ccc(-c2ccc(-n3c4ccccc4c4cc(-n5c6ccccc6c6c([Si](c7ccccc7)(c7ccccc7)c7cccc8c7oc7ccccc78)cccc65)ccc43)cc2)cc1 ; c1ccc(-c2ccc(-n3c4ccccc4c4c(-n5c6ccccc6c6c([Si](c7ccccc7)(c7ccccc7)c7ccc8oc9ccccc9c8c7)cccc65)cccc43)cc2)cc1]

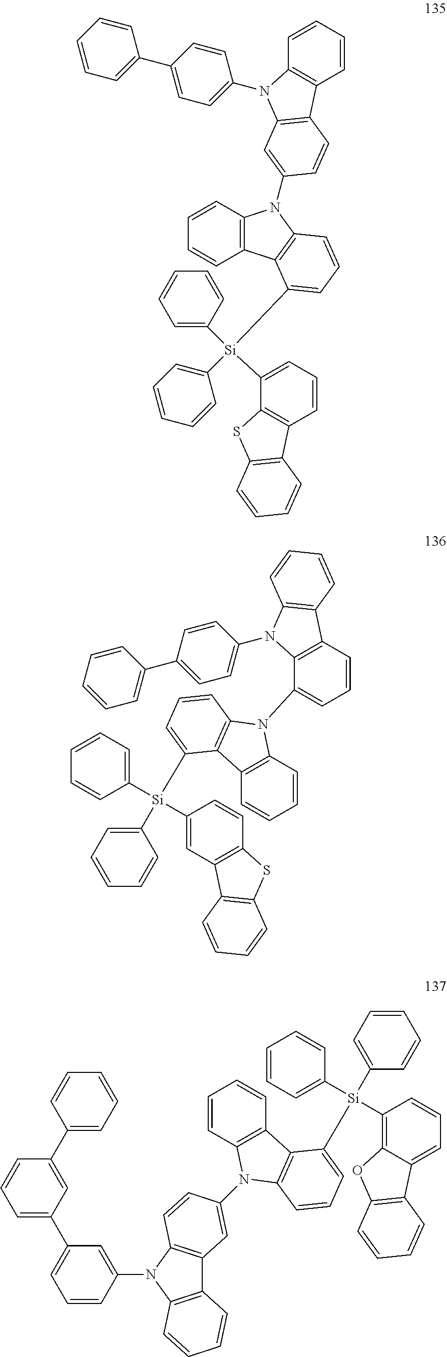

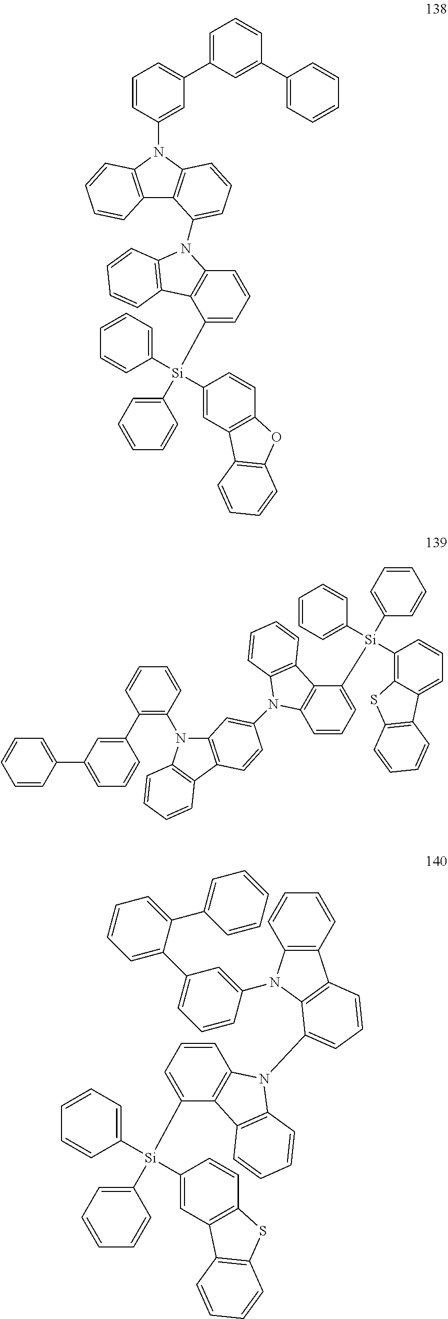 [SMILES: c1ccc(-c2cccc(-c3cccc(-n4c5ccccc5c5c(-n6c7ccccc7c7c([Si](c8ccccc8)(c8ccccc8)c8ccc9oc%10ccccc%10c9c8)cccc76)cccc54)c3)c2)cc1 ; c1ccc(-c2cccc(-c3ccccc3-n3c4ccccc4c4ccc(-n5c6ccccc6c6c([Si](c7ccccc7)(c7ccccc7)c7cccc8c7sc7ccccc78)cccc65)cc43)c2)cc1 ; c1ccc(-c2ccccc2-c2cccc(-n3c4ccccc4c4cccc(-n5c6ccccc6c6c([Si](c7ccccc7)(c7ccccc7)c7ccc8sc9ccccc9c8c7)cccc65)c43)c2)cc1]

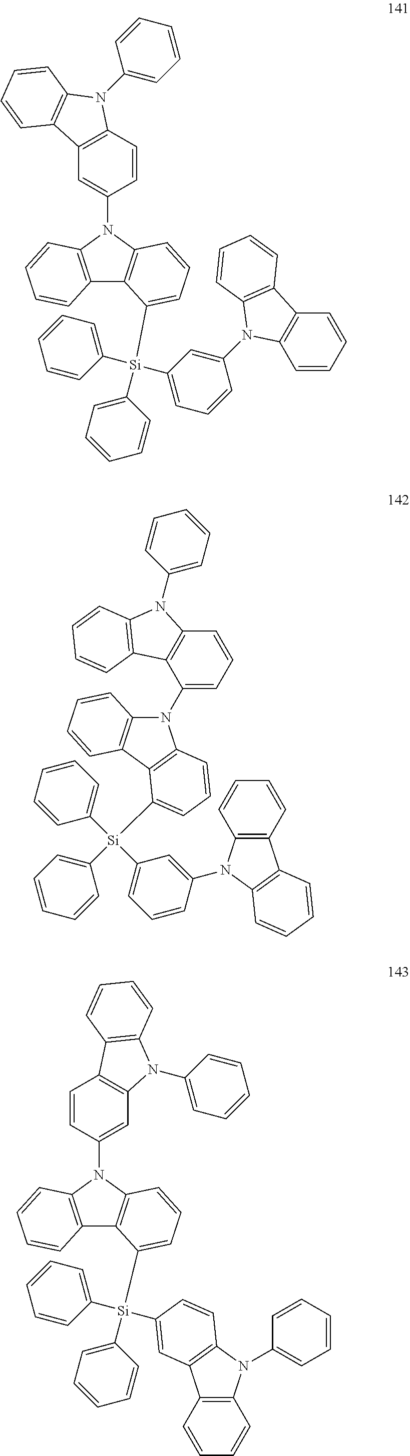 [SMILES: c1ccc(-n2c3ccccc3c3cc(-n4c5ccccc5c5c([Si](c6ccccc6)(c6ccccc6)c6cccc(-n7c8ccccc8c8ccccc87)c6)cccc54)ccc32)cc1 ; c1ccc(-n2c3ccccc3c3c(-n4c5ccccc5c5c([Si](c6ccccc6)(c6ccccc6)c6cccc(-n7c8ccccc8c8ccccc87)c6)cccc54)cccc32)cc1 ; c1ccc(-n2c3ccccc3c3cc([Si](c4ccccc4)(c4ccccc4)c4cccc5c4c4ccccc4n5-c4ccc5c6ccccc6n(-c6ccccc6)c5c4)ccc32)cc1]

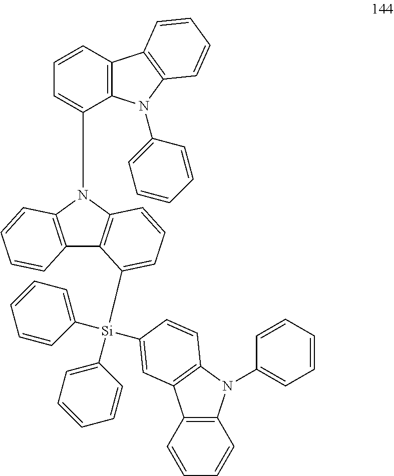 [SMILES: c1ccc(-n2c3ccccc3c3cc([Si](c4ccccc4)(c4ccccc4)c4cccc5c4c4ccccc4n5-c4cccc5c6ccccc6n(-c6ccccc6)c45)ccc32)cc1]

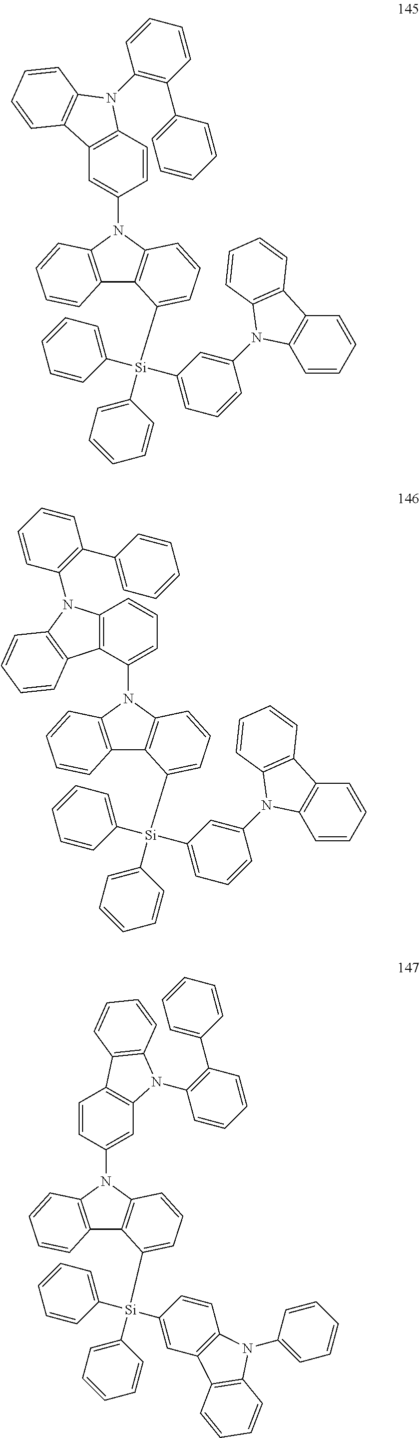 [SMILES: c1ccc(-c2ccccc2-n2c3ccccc3c3cc(-n4c5ccccc5c5c([Si](c6ccccc6)(c6ccccc6)c6cccc(-n7c8ccccc8c8ccccc87)c6)cccc54)ccc32)cc1 ; c1ccc(-c2ccccc2-n2c3ccccc3c3c(-n4c5ccccc5c5c([Si](c6ccccc6)(c6ccccc6)c6cccc(-n7c8ccccc8c8ccccc87)c6)cccc54)cccc32)cc1 ; c1ccc(-c2ccccc2-n2c3ccccc3c3ccc(-n4c5ccccc5c5c([Si](c6ccccc6)(c6ccccc6)c6ccc7c(c6)c6ccccc6n7-c6ccccc6)cccc54)cc32)cc1]

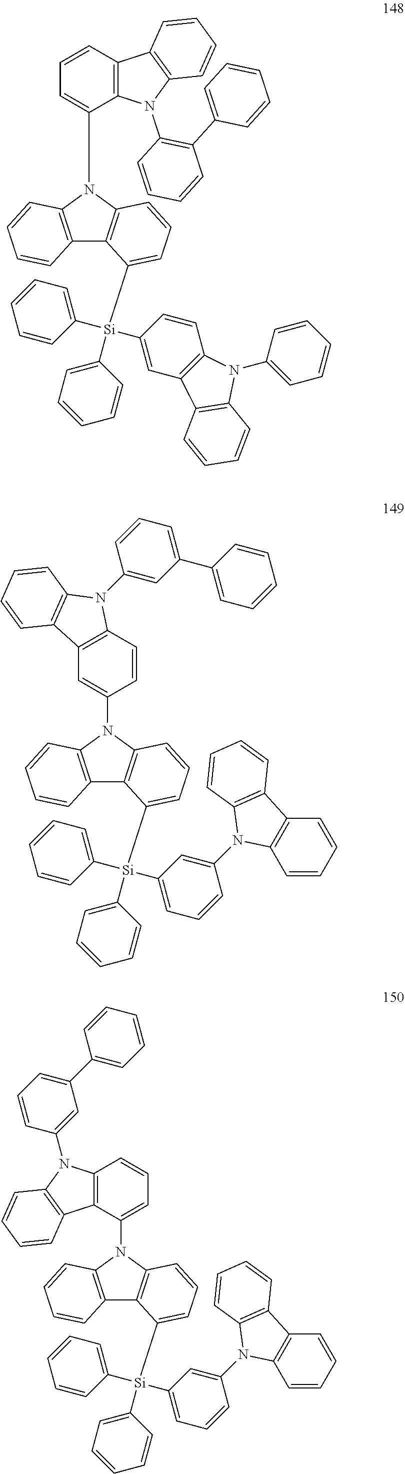

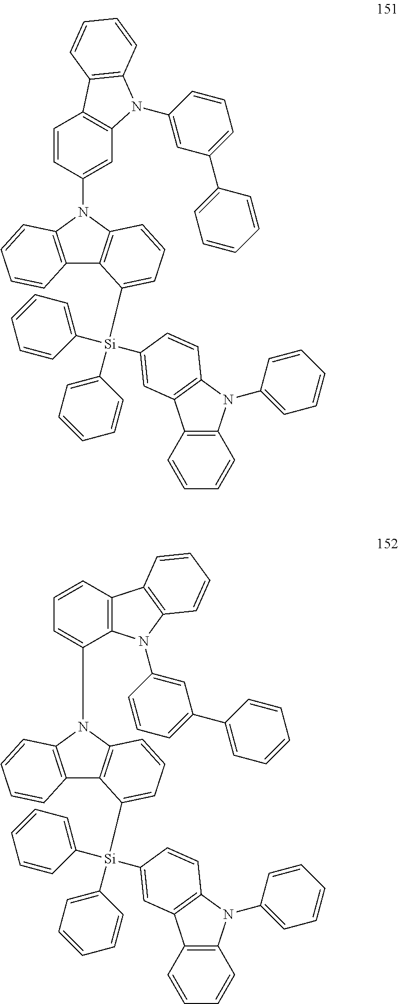 [SMILES: c1ccc(-c2cccc(-n3c4ccccc4c4ccc(-n5c6ccccc6c6c([Si](c7ccccc7)(c7ccccc7)c7ccc8c(c7)c7ccccc7n8-c7ccccc7)cccc65)cc43)c2)cc1 ; c1ccc(-c2cccc(-n3c4ccccc4c4cccc(-n5c6ccccc6c6c([Si](c7ccccc7)(c7ccccc7)c7ccc8c(c7)c7ccccc7n8-c7ccccc7)cccc65)c43)c2)cc1]

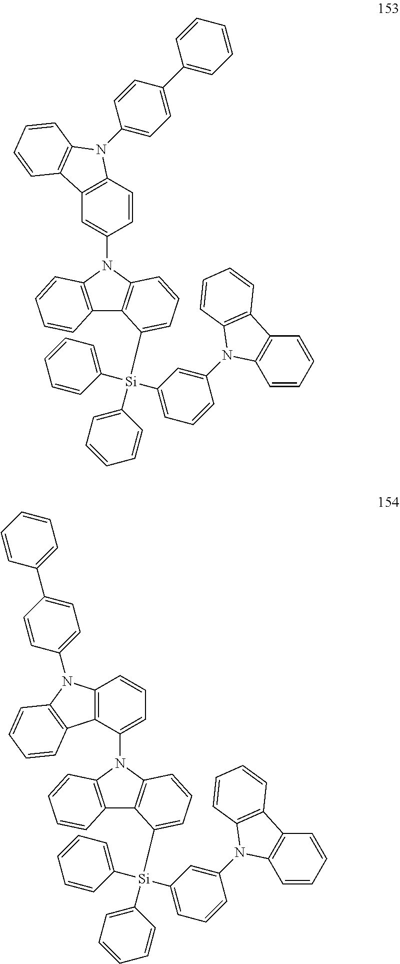 [SMILES: c1ccc(-c2ccc(-n3c4ccccc4c4cc(-n5c6ccccc6c6c([Si](c7ccccc7)(c7ccccc7)c7cccc(-n8c9ccccc9c9ccccc98)c7)cccc65)ccc43)cc2)cc1 ; c1ccc(-c2ccc(-n3c4ccccc4c4c(-n5c6ccccc6c6c([Si](c7ccccc7)(c7ccccc7)c7cccc(-n8c9ccccc9c9ccccc98)c7)cccc65)cccc43)cc2)cc1]

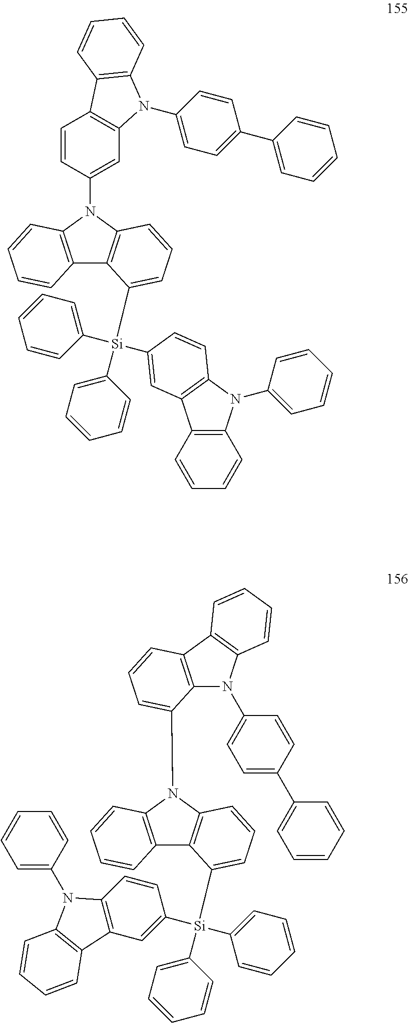 [SMILES: c1ccc(-c2ccc(-n3c4ccccc4c4ccc(-n5c6ccccc6c6c([Si](c7ccccc7)(c7ccccc7)c7ccc8c(c7)c7ccccc7n8-c7ccccc7)cccc65)cc43)cc2)cc1 ; c1ccc(-c2ccc(-n3c4ccccc4c4cccc(-n5c6ccccc6c6c([Si](c7ccccc7)(c7ccccc7)c7ccc8c(c7)c7ccccc7n8-c7ccccc7)cccc65)c43)cc2)cc1]

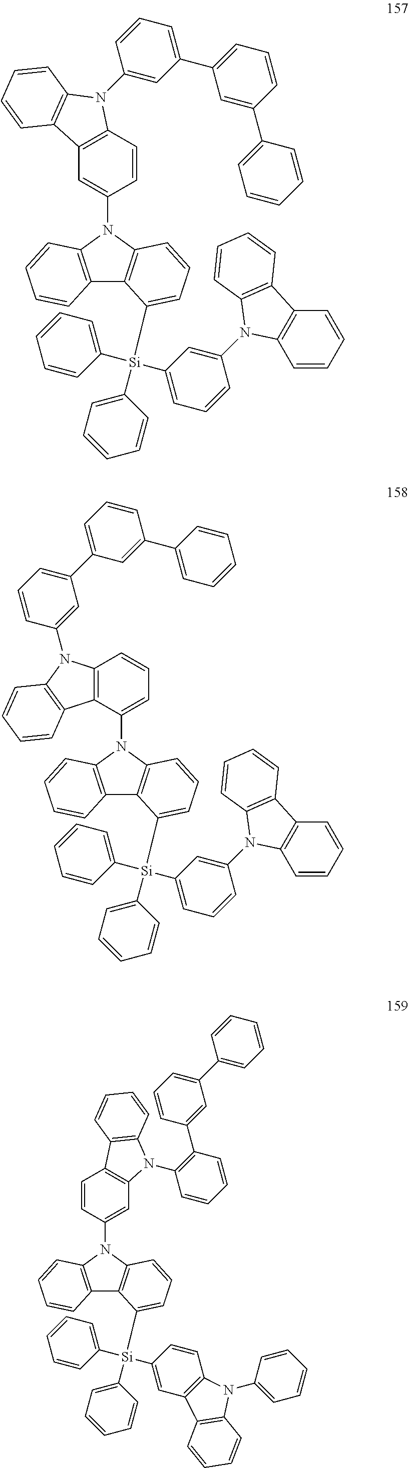

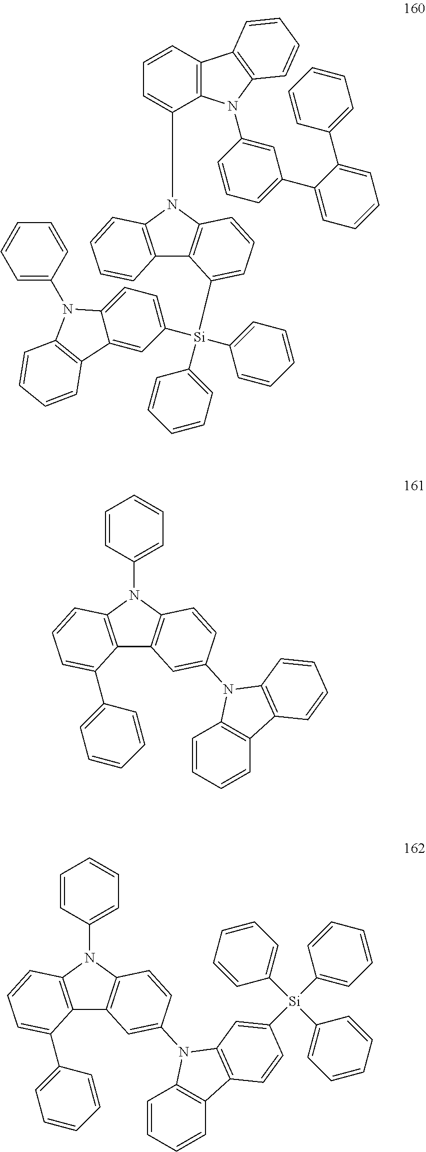 [SMILES: c1ccc(-c2ccccc2-c2cccc(-n3c4ccccc4c4cccc(-n5c6ccccc6c6c([Si](c7ccccc7)(c7ccccc7)c7ccc8c(c7)c7ccccc7n8-c7ccccc7)cccc65)c43)c2)cc1 ; c1ccc(-c2cccc3c2c2cc(-n4c5ccccc5c5ccccc54)ccc2n3-c2ccccc2)cc1 ; c1ccc(-c2cccc3c2c2cc(-n4c5ccccc5c5ccc([Si](c6ccccc6)(c6ccccc6)c6ccccc6)cc54)ccc2n3-c2ccccc2)cc1]

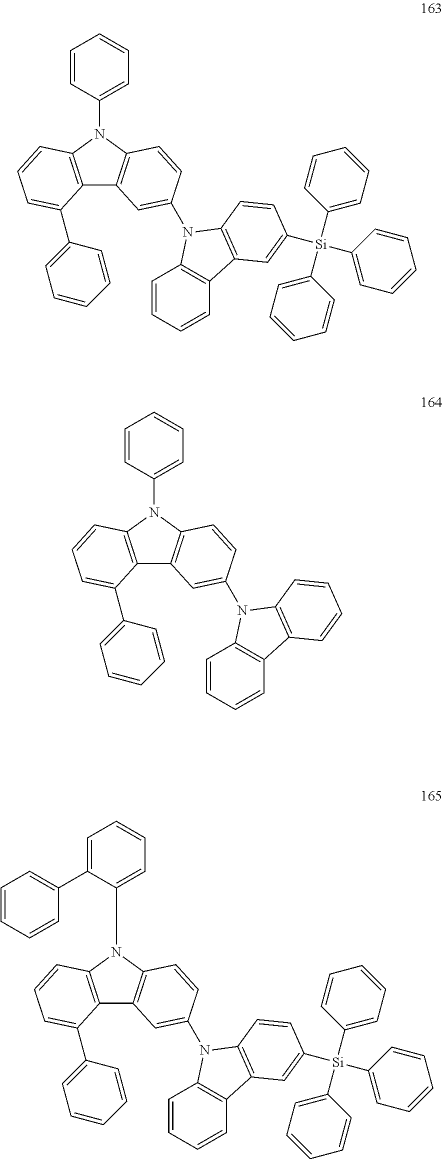 [SMILES: c1ccc(-c2cccc3c2c2cc(-n4c5ccccc5c5cc([Si](c6ccccc6)(c6ccccc6)c6ccccc6)ccc54)ccc2n3-c2ccccc2)cc1 ; c1ccc(-c2cccc3c2c2cc(-n4c5ccccc5c5ccccc54)ccc2n3-c2ccccc2)cc1 ; c1ccc(-c2ccccc2-n2c3ccc(-n4c5ccccc5c5cc([Si](c6ccccc6)(c6ccccc6)c6ccccc6)ccc54)cc3c3c(-c4ccccc4)cccc32)cc1]

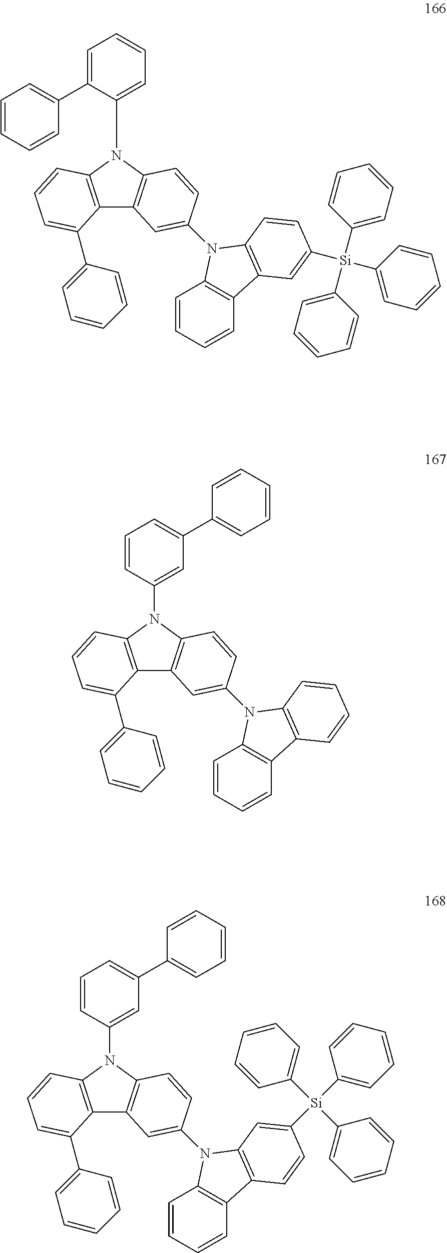 [SMILES: c1ccc(-c2ccccc2-n2c3ccc(-n4c5ccccc5c5cc([Si](c6ccccc6)(c6ccccc6)c6ccccc6)ccc54)cc3c3c(-c4ccccc4)cccc32)cc1 ; c1ccc(-c2cccc(-n3c4ccc(-n5c6ccccc6c6ccccc65)cc4c4c(-c5ccccc5)cccc43)c2)cc1 ; c1ccc(-c2cccc(-n3c4ccc(-n5c6ccccc6c6ccc([Si](c7ccccc7)(c7ccccc7)c7ccccc7)cc65)cc4c4c(-c5ccccc5)cccc43)c2)cc1]

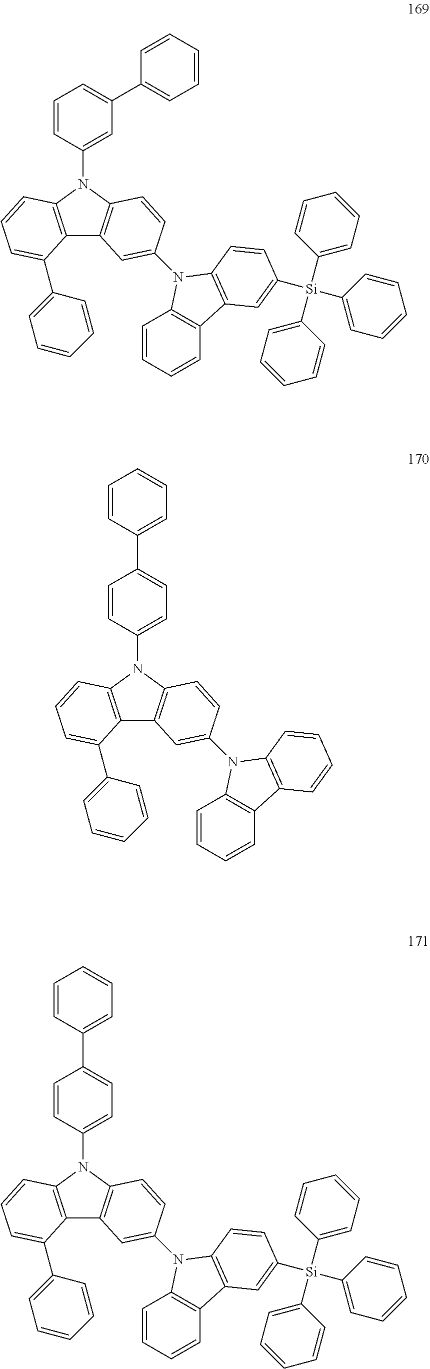 [SMILES: c1ccc(-c2cccc(-n3c4ccc(-n5c6ccccc6c6cc([Si](c7ccccc7)(c7ccccc7)c7ccccc7)ccc65)cc4c4c(-c5ccccc5)cccc43)c2)cc1 ; c1ccc(-c2ccc(-n3c4ccc(-n5c6ccccc6c6ccccc65)cc4c4c(-c5ccccc5)cccc43)cc2)cc1 ; c1ccc(-c2ccc(-n3c4ccc(-n5c6ccccc6c6cc([Si](c7ccccc7)(c7ccccc7)c7ccccc7)ccc65)cc4c4c(-c5ccccc5)cccc43)cc2)cc1]

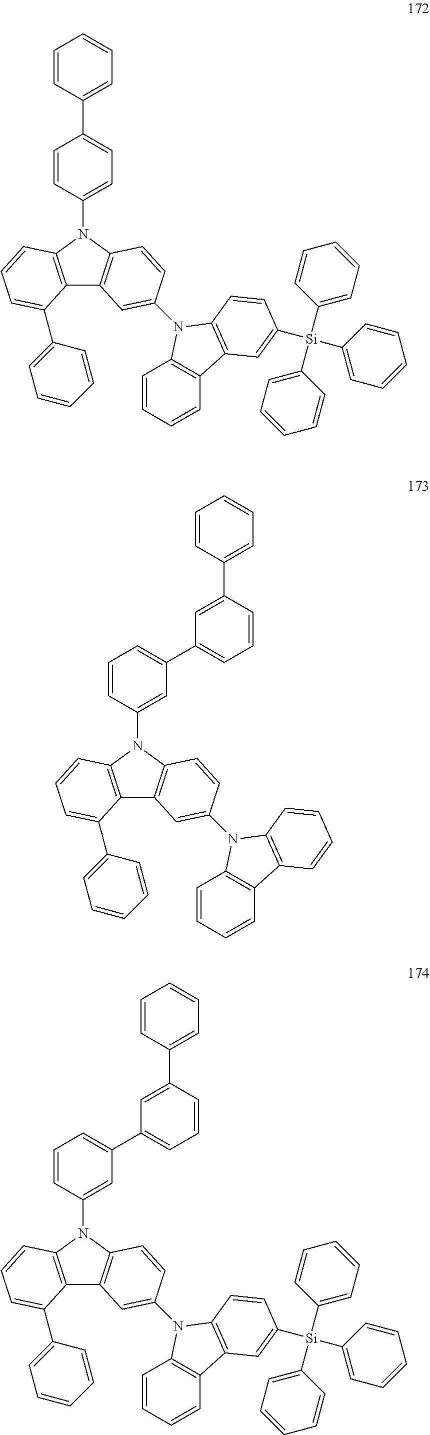 [SMILES: c1ccc(-c2ccc(-n3c4ccc(-n5c6ccccc6c6cc([Si](c7ccccc7)(c7ccccc7)c7ccccc7)ccc65)cc4c4c(-c5ccccc5)cccc43)cc2)cc1 ; c1ccc(-c2cccc(-c3cccc(-n4c5ccc(-n6c7ccccc7c7ccccc76)cc5c5c(-c6ccccc6)cccc54)c3)c2)cc1 ; c1ccc(-c2cccc(-c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1]

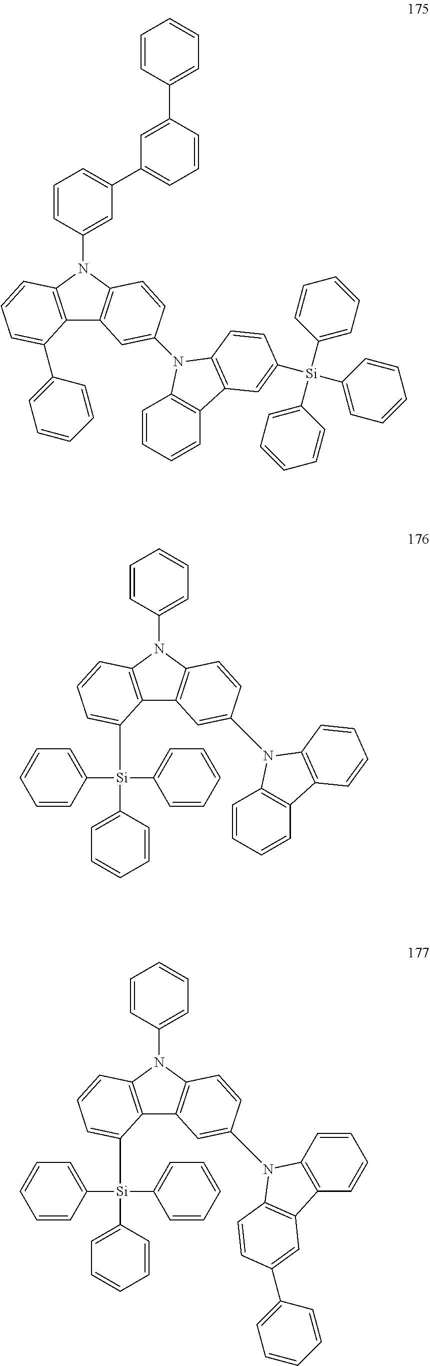 [SMILES: c1ccc(-c2cccc(-c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1 ; c1ccc(-n2c3ccc(-n4c5ccccc5c5ccccc54)cc3c3c([Si](c4ccccc4)(c4ccccc4)c4ccccc4)cccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2ccccc2)cc1]

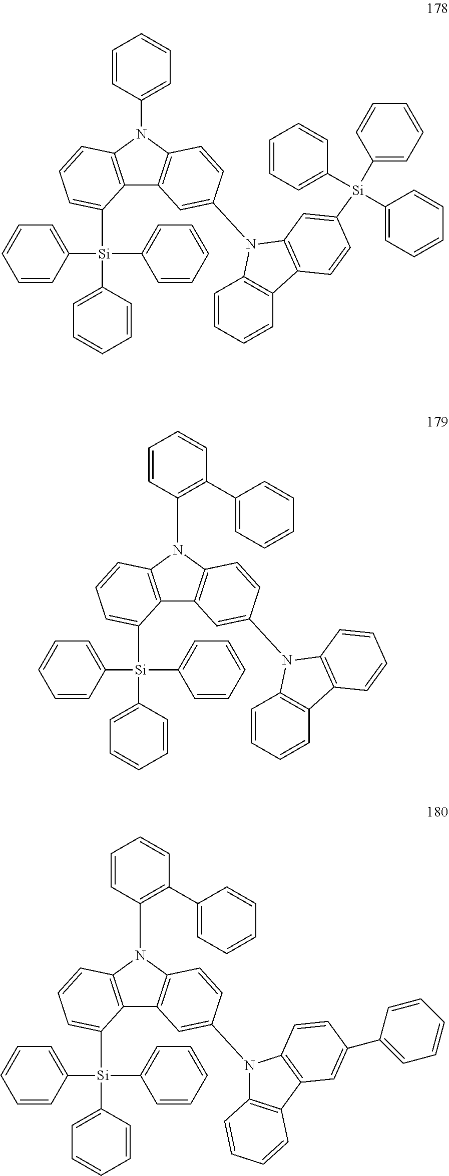 [SMILES: c1ccc(-n2c3ccc(-n4c5ccccc5c5ccc([Si](c6ccccc6)(c6ccccc6)c6ccccc6)cc54)cc3c3c([Si](c4ccccc4)(c4ccccc4)c4ccccc4)cccc32)cc1 ; c1ccc(-c2ccccc2-n2c3ccc(-n4c5ccccc5c5ccccc54)cc3c3c([Si](c4ccccc4)(c4ccccc4)c4ccccc4)cccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2ccccc2-c2ccccc2)cc1]

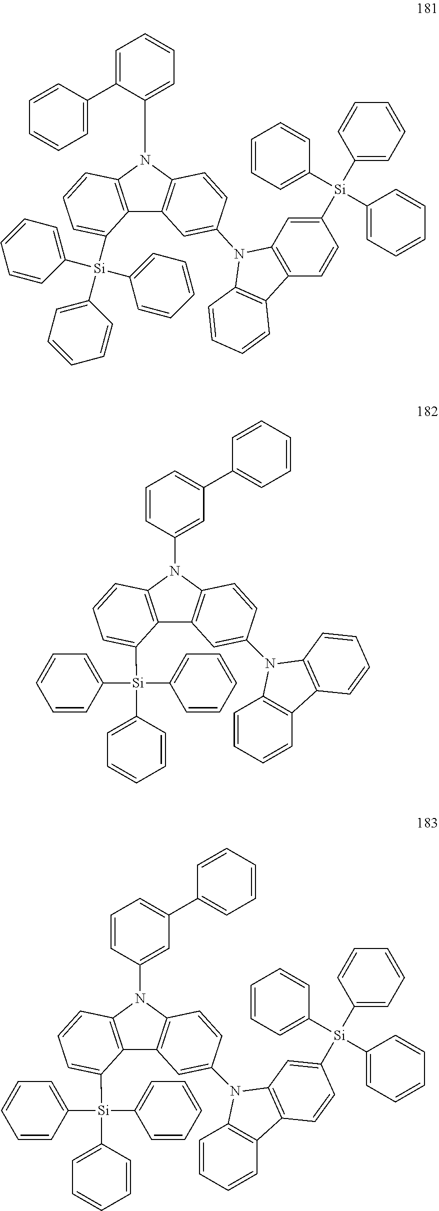 [SMILES: c1ccc(-c2ccccc2-n2c3ccc(-n4c5ccccc5c5ccc([Si](c6ccccc6)(c6ccccc6)c6ccccc6)cc54)cc3c3c([Si](c4ccccc4)(c4ccccc4)c4ccccc4)cccc32)cc1 ; c1ccc(-c2cccc(-n3c4ccc(-n5c6ccccc6c6ccccc65)cc4c4c([Si](c5ccccc5)(c5ccccc5)c5ccccc5)cccc43)c2)cc1 ; c1ccc(-c2cccc(-n3c4ccc(-n5c6ccccc6c6ccc([Si](c7ccccc7)(c7ccccc7)c7ccccc7)cc65)cc4c4c([Si](c5ccccc5)(c5ccccc5)c5ccccc5)cccc43)c2)cc1]

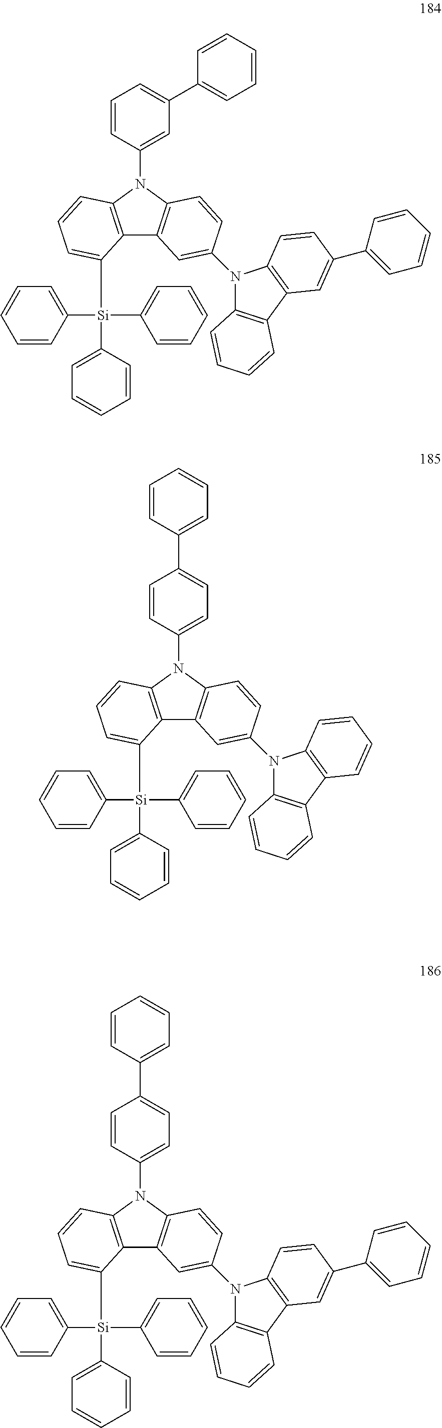 [SMILES: c1ccc(-c2cccc(-n3c4ccc(-n5c6ccccc6c6cc(-c7ccccc7)ccc65)cc4c4c([Si](c5ccccc5)(c5ccccc5)c5ccccc5)cccc43)c2)cc1 ; c1ccc(-c2ccc(-n3c4ccc(-n5c6ccccc6c6ccccc65)cc4c4c([Si](c5ccccc5)(c5ccccc5)c5ccccc5)cccc43)cc2)cc1 ; c1ccc(-c2ccc(-n3c4ccc(-n5c6ccccc6c6cc(-c7ccccc7)ccc65)cc4c4c([Si](c5ccccc5)(c5ccccc5)c5ccccc5)cccc43)cc2)cc1]

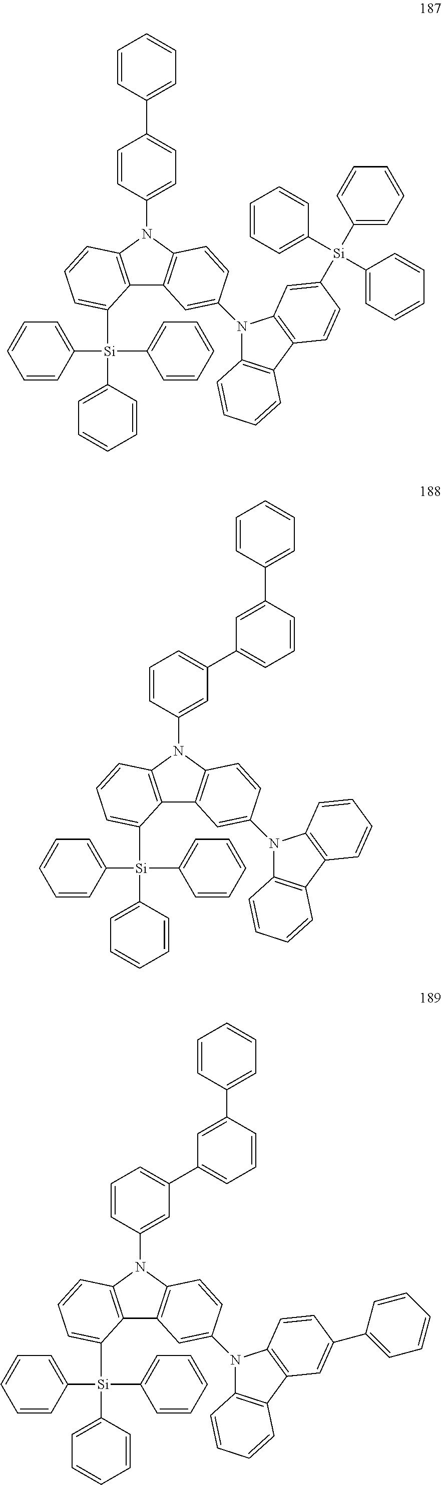 [SMILES: c1ccc(-c2ccc(-n3c4ccc(-n5c6ccccc6c6ccc([Si](c7ccccc7)(c7ccccc7)c7ccccc7)cc65)cc4c4c([Si](c5ccccc5)(c5ccccc5)c5ccccc5)cccc43)cc2)cc1 ; c1ccc(-c2cccc(-c3cccc(-n4c5ccc(-n6c7ccccc7c7ccccc76)cc5c5c([Si](c6ccccc6)(c6ccccc6)c6ccccc6)cccc54)c3)c2)cc1 ; c1ccc(-c2cccc(-c3cccc(-n4c5ccc(-n6c7ccccc7c7cc(-c8ccccc8)ccc76)cc5c5c([Si](c6ccccc6)(c6ccccc6)c6ccccc6)cccc54)c3)c2)cc1]

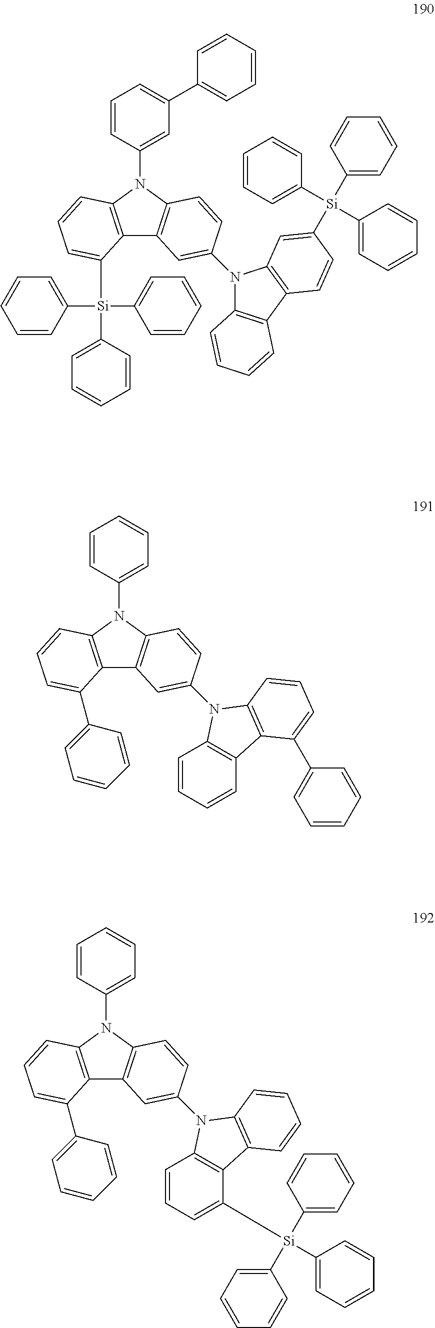 [SMILES: c1ccc(-c2cccc(-n3c4ccc(-n5c6ccccc6c6ccc([Si](c7ccccc7)(c7ccccc7)c7ccccc7)cc65)cc4c4c([Si](c5ccccc5)(c5ccccc5)c5ccccc5)cccc43)c2)cc1 ; c1ccc(-c2cccc3c2c2ccccc2n3-c2ccc3c(c2)c2c(-c4ccccc4)cccc2n3-c2ccccc2)cc1 ; c1ccc(-c2cccc3c2c2cc(-n4c5ccccc5c5c([Si](c6ccccc6)(c6ccccc6)c6ccccc6)cccc54)ccc2n3-c2ccccc2)cc1]

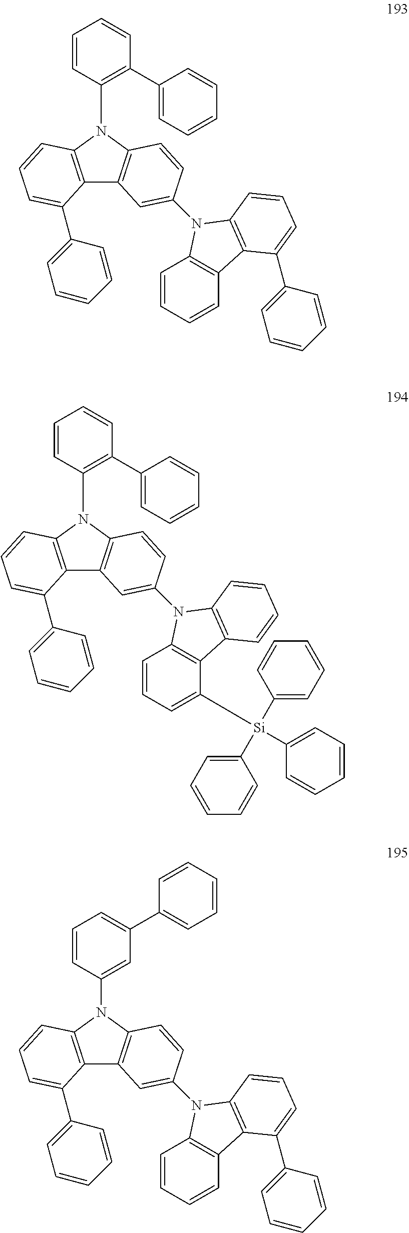 [SMILES: c1ccc(-c2ccccc2-n2c3ccc(-n4c5ccccc5c5c(-c6ccccc6)cccc54)cc3c3c(-c4ccccc4)cccc32)cc1 ; c1ccc(-c2ccccc2-n2c3ccc(-n4c5ccccc5c5c([Si](c6ccccc6)(c6ccccc6)c6ccccc6)cccc54)cc3c3c(-c4ccccc4)cccc32)cc1 ; c1ccc(-c2cccc(-n3c4ccc(-n5c6ccccc6c6c(-c7ccccc7)cccc65)cc4c4c(-c5ccccc5)cccc43)c2)cc1]

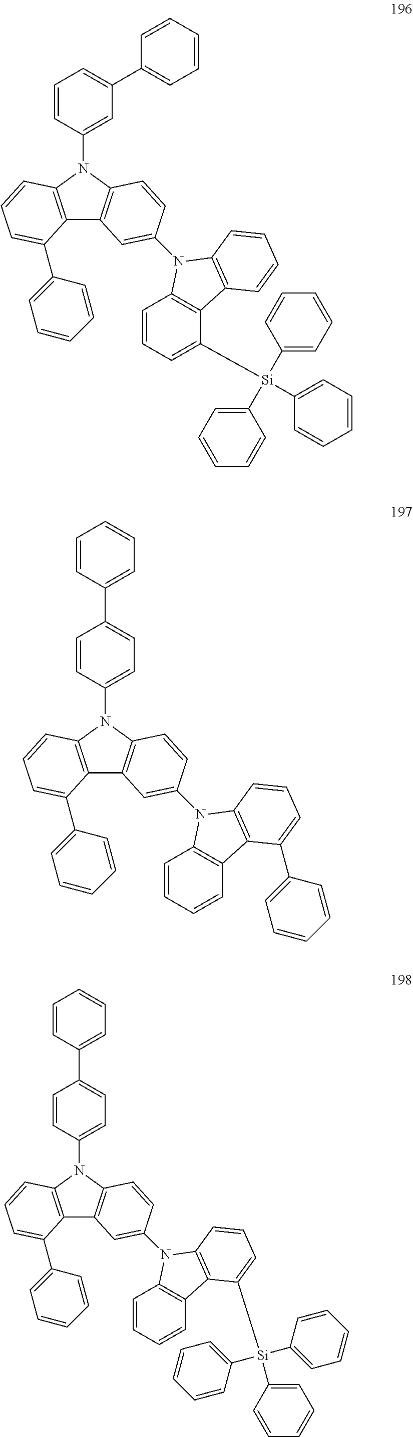 [SMILES: c1ccc(-c2cccc(-n3c4ccc(-n5c6ccccc6c6c([Si](c7ccccc7)(c7ccccc7)c7ccccc7)cccc65)cc4c4c(-c5ccccc5)cccc43)c2)cc1 ; c1ccc(-c2ccc(-n3c4ccc(-n5c6ccccc6c6c(-c7ccccc7)cccc65)cc4c4c(-c5ccccc5)cccc43)cc2)cc1 ; c1ccc(-c2ccc(-n3c4ccc(-n5c6ccccc6c6c([Si](c7ccccc7)(c7ccccc7)c7ccccc7)cccc65)cc4c4c(-c5ccccc5)cccc43)cc2)cc1]

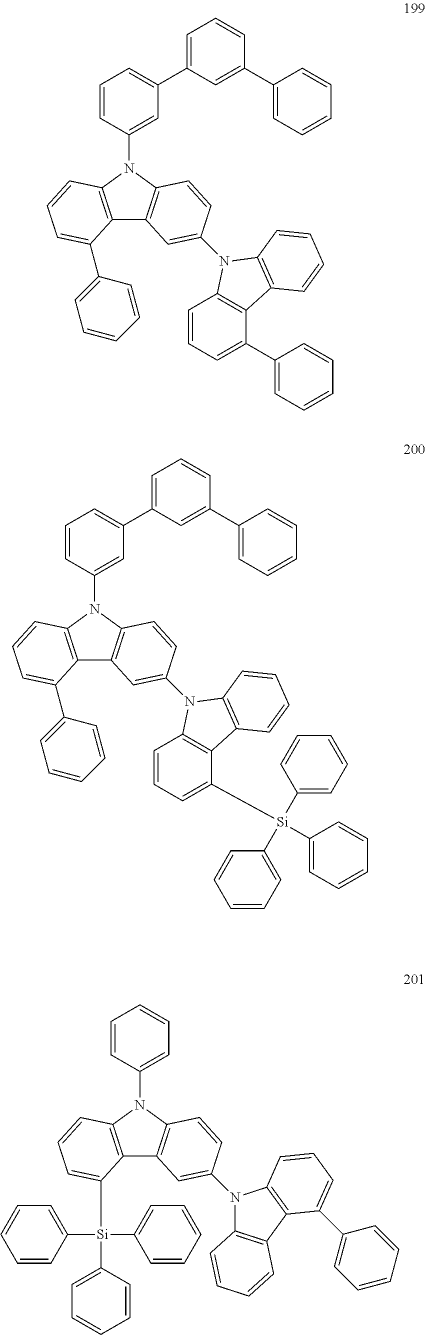 [SMILES: c1ccc(-c2cccc(-c3cccc(-n4c5ccc(-n6c7ccccc7c7c(-c8ccccc8)cccc76)cc5c5c(-c6ccccc6)cccc54)c3)c2)cc1 ; c1ccc(-c2cccc(-c3cccc(-n4c5ccc(-n6c7ccccc7c7c([Si](c8ccccc8)(c8ccccc8)c8ccccc8)cccc76)cc5c5c(-c6ccccc6)cccc54)c3)c2)cc1 ; c1ccc(-c2cccc3c2c2ccccc2n3-c2ccc3c(c2)c2c([Si](c4ccccc4)(c4ccccc4)c4ccccc4)cccc2n3-c2ccccc2)cc1]

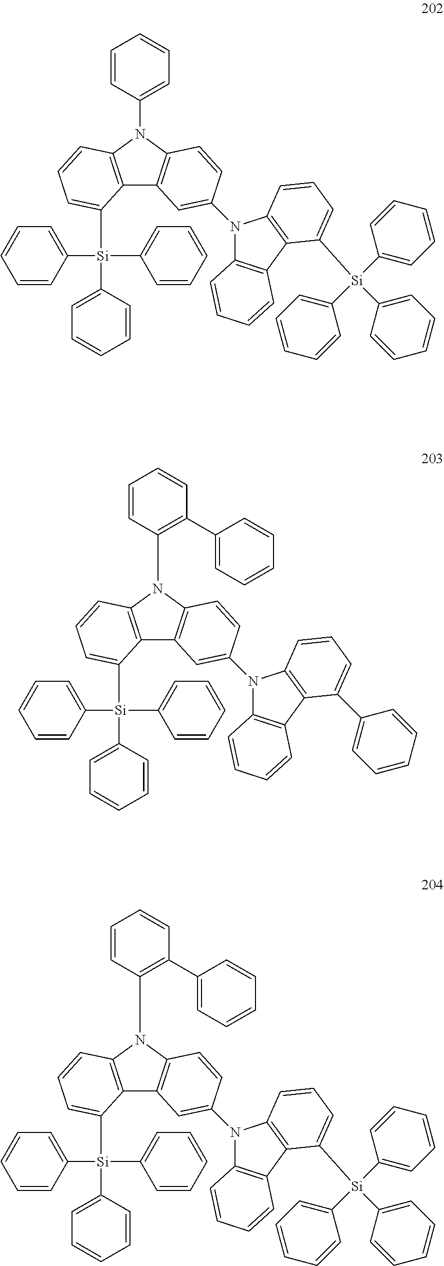 [SMILES: c1ccc(-n2c3ccc(-n4c5ccccc5c5c([Si](c6ccccc6)(c6ccccc6)c6ccccc6)cccc54)cc3c3c([Si](c4ccccc4)(c4ccccc4)c4ccccc4)cccc32)cc1 ; c1ccc(-c2ccccc2-n2c3ccc(-n4c5ccccc5c5c(-c6ccccc6)cccc54)cc3c3c([Si](c4ccccc4)(c4ccccc4)c4ccccc4)cccc32)cc1 ; c1ccc(-c2ccccc2-n2c3ccc(-n4c5ccccc5c5c([Si](c6ccccc6)(c6ccccc6)c6ccccc6)cccc54)cc3c3c([Si](c4ccccc4)(c4ccccc4)c4ccccc4)cccc32)cc1]

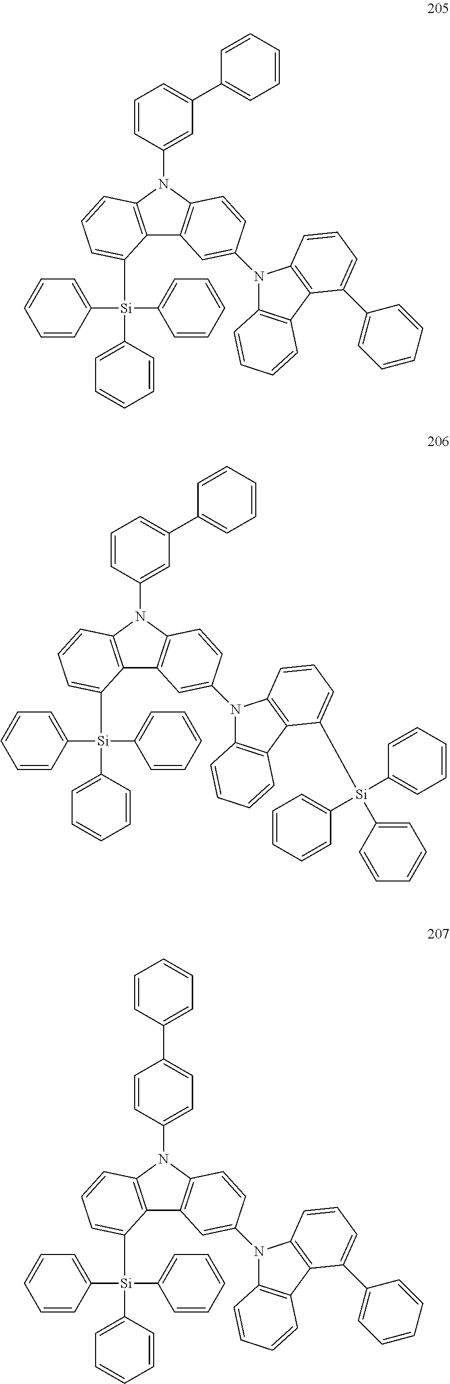 [SMILES: c1ccc(-c2cccc(-n3c4ccc(-n5c6ccccc6c6c(-c7ccccc7)cccc65)cc4c4c([Si](c5ccccc5)(c5ccccc5)c5ccccc5)cccc43)c2)cc1 ; c1ccc(-c2cccc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)c2)cc1 ; c1ccc(-c2ccc(-n3c4ccc(-n5c6ccccc6c6c(-c7ccccc7)cccc65)cc4c4c([Si](c5ccccc5)(c5ccccc5)c5ccccc5)cccc43)cc2)cc1]

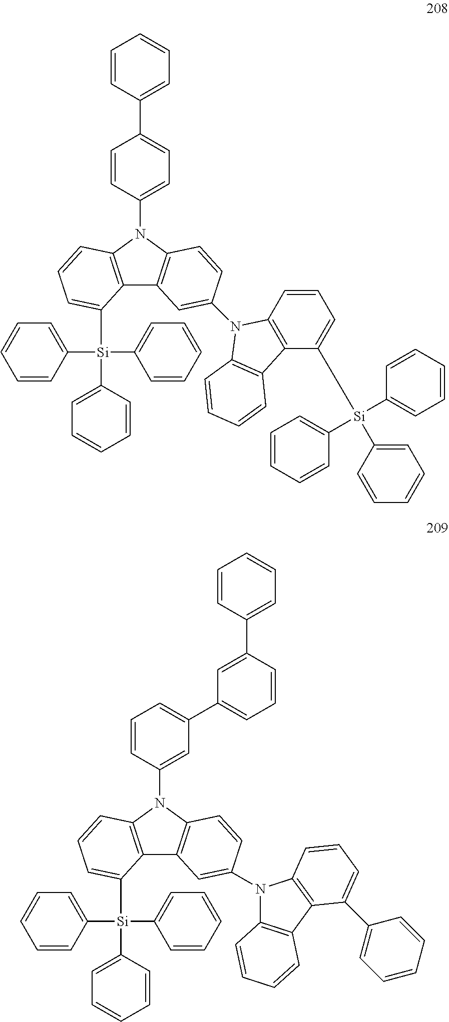 [SMILES: c1ccc(-c2ccc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)cc2)cc1 ; c1ccc(-c2cccc(-c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1]

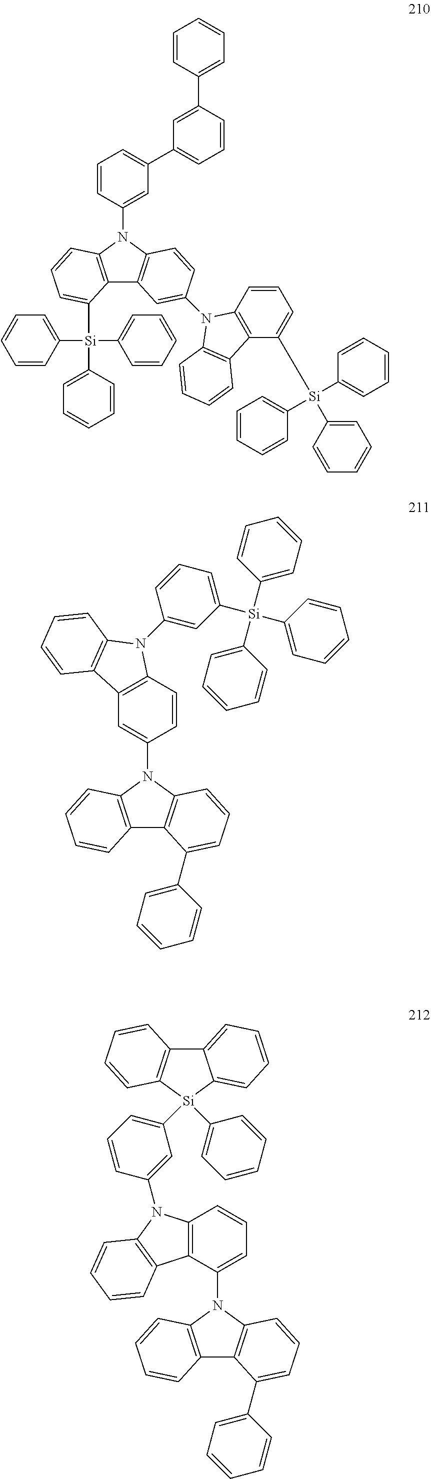 [SMILES: c1ccc(-c2cccc(-c3cccc(-n4c5ccc(-n6c7ccccc7c7c([Si](c8ccccc8)(c8ccccc8)c8ccccc8)cccc76)cc5c5c([Si](c6ccccc6)(c6ccccc6)c6ccccc6)cccc54)c3)c2)cc1 ; c1ccc(-c2cccc3c2c2ccccc2n3-c2ccc3c(c2)c2ccccc2n3-c2cccc([Si](c3ccccc3)(c3ccccc3)c3ccccc3)c2)cc1 ; c1ccc(-c2cccc3c2c2ccccc2n3-c2cccc3c2c2ccccc2n3-c2cccc([Si]3(c4ccccc4)c4ccccc4-c4ccccc43)c2)cc1]

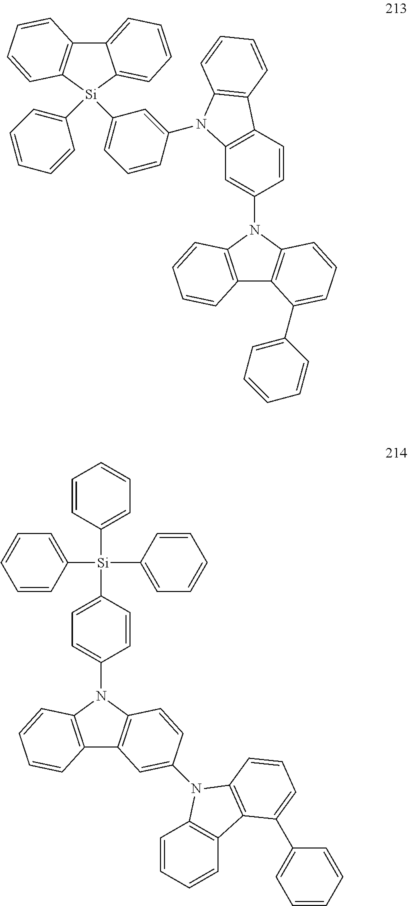 [SMILES: c1ccc(-c2cccc3c2c2ccccc2n3-c2ccc3c4ccccc4n(-c4cccc([Si]5(c6ccccc6)c6ccccc6-c6ccccc65)c4)c3c2)cc1 ; c1ccc(-c2cccc3c2c2ccccc2n3-c2ccc3c(c2)c2ccccc2n3-c2ccc([Si](c3ccccc3)(c3ccccc3)c3ccccc3)cc2)cc1]

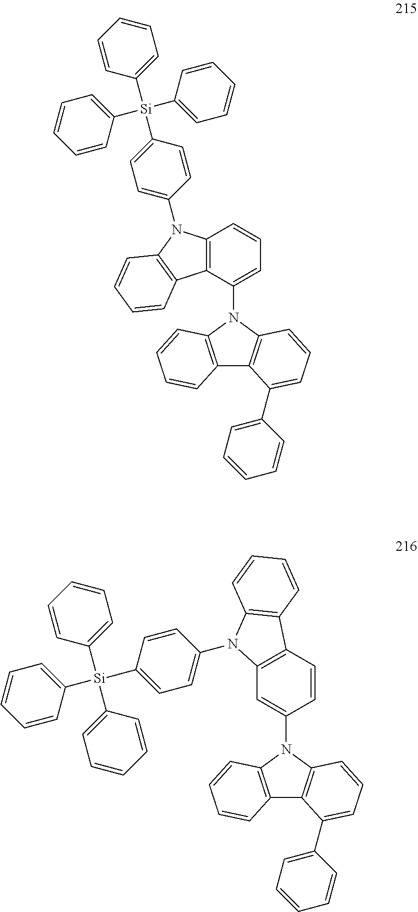 [SMILES: c1ccc(-c2cccc3c2c2ccccc2n3-c2cccc3c2c2ccccc2n3-c2ccc([Si](c3ccccc3)(c3ccccc3)c3ccccc3)cc2)cc1 ; c1ccc(-c2cccc3c2c2ccccc2n3-c2ccc3c4ccccc4n(-c4ccc([Si](c5ccccc5)(c5ccccc5)c5ccccc5)cc4)c3c2)cc1]

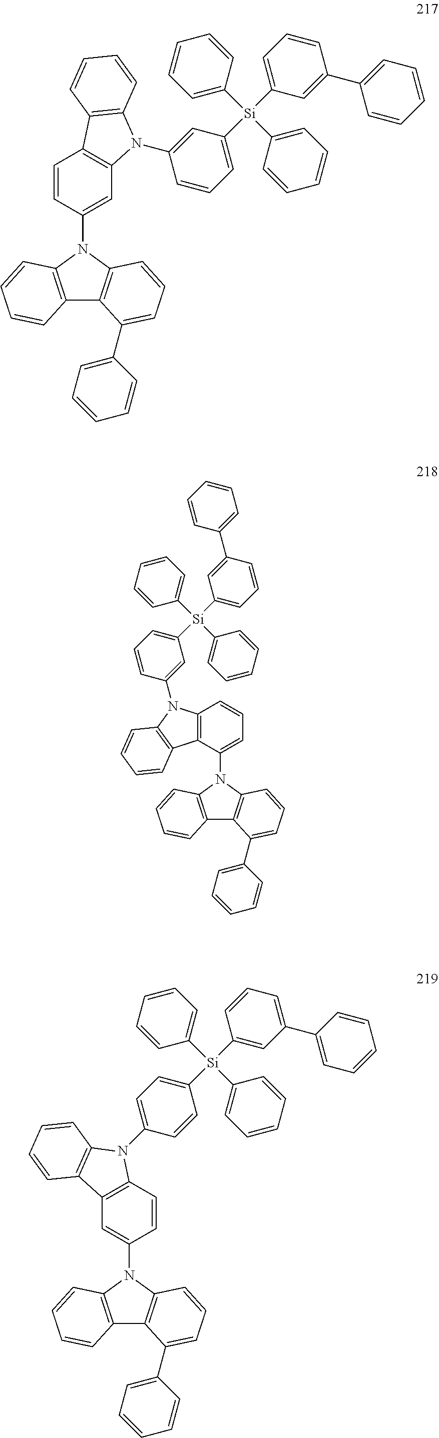 [SMILES: c1ccc(-c2cccc([Si](c3ccccc3)(c3ccccc3)c3cccc(-n4c5ccccc5c5ccc(-n6c7ccccc7c7c(-c8ccccc8)cccc76)cc54)c3)c2)cc1 ; c1ccc(-c2cccc([Si](c3ccccc3)(c3ccccc3)c3cccc(-n4c5ccccc5c5c(-n6c7ccccc7c7c(-c8ccccc8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c(-c8ccccc8)cccc76)ccc54)cc3)c2)cc1]

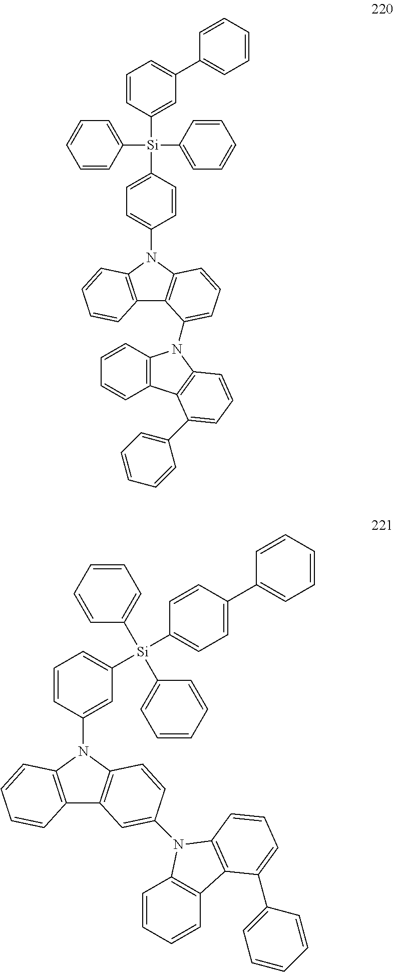 [SMILES: c1ccc(-c2cccc([Si](c3ccccc3)(c3ccccc3)c3ccc(-n4c5ccccc5c5c(-n6c7ccccc7c7c(-c8ccccc8)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c(-c8ccccc8)cccc76)ccc54)c3)cc2)cc1]

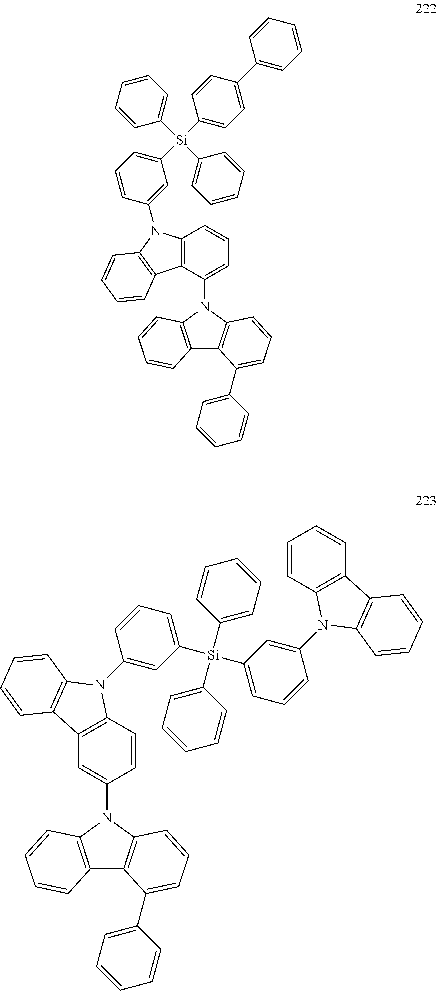 [SMILES: c1ccc(-c2ccc([Si](c3ccccc3)(c3ccccc3)c3cccc(-n4c5ccccc5c5c(-n6c7ccccc7c7c(-c8ccccc8)cccc76)cccc54)c3)cc2)cc1 ; c1ccc(-c2cccc3c2c2ccccc2n3-c2ccc3c(c2)c2ccccc2n3-c2cccc([Si](c3ccccc3)(c3ccccc3)c3cccc(-n4c5ccccc5c5ccccc54)c3)c2)cc1]

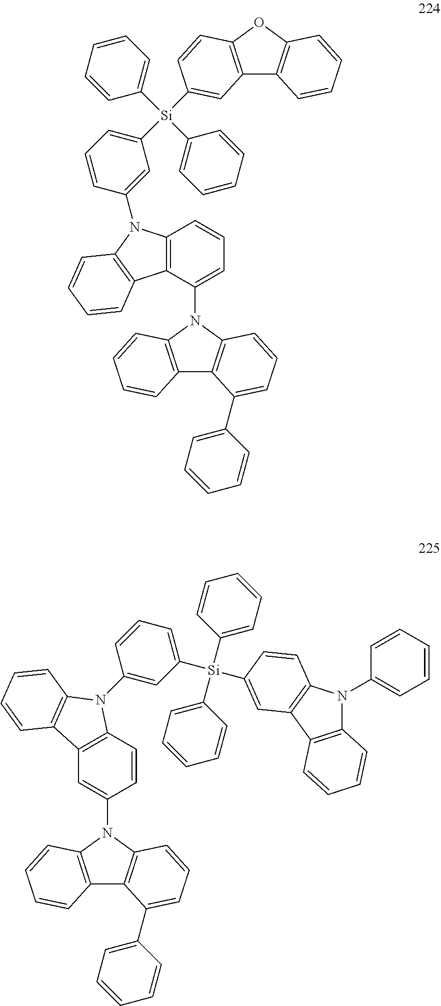 [SMILES: c1ccc(-c2cccc3c2c2ccccc2n3-c2cccc3c2c2ccccc2n3-c2cccc([Si](c3ccccc3)(c3ccccc3)c3ccc4oc5ccccc5c4c3)c2)cc1 ; c1ccc(-c2cccc3c2c2ccccc2n3-c2ccc3c(c2)c2ccccc2n3-c2cccc([Si](c3ccccc3)(c3ccccc3)c3ccc4c(c3)c3ccccc3n4-c3ccccc3)c2)cc1]

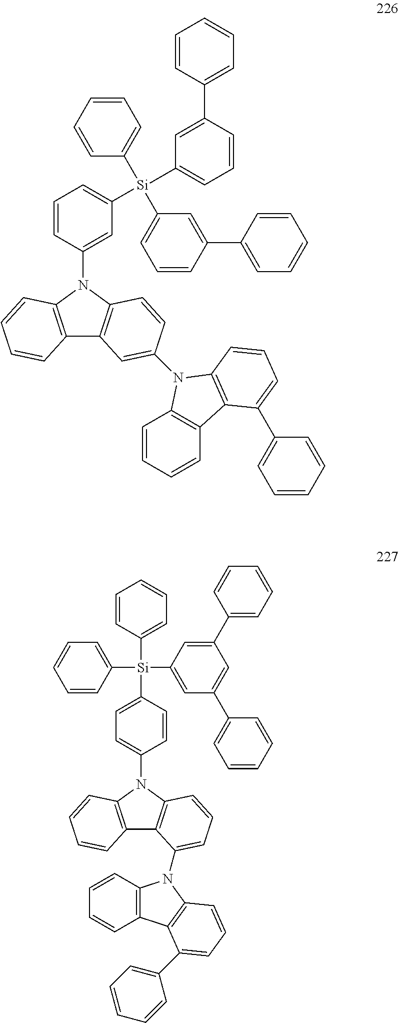 [SMILES: c1ccc(-c2cccc([Si](c3ccccc3)(c3cccc(-c4ccccc4)c3)c3cccc(-n4c5ccccc5c5cc(-n6c7ccccc7c7c(-c8ccccc8)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c(-c8ccccc8)cccc76)cccc54)cc3)c2)cc1]

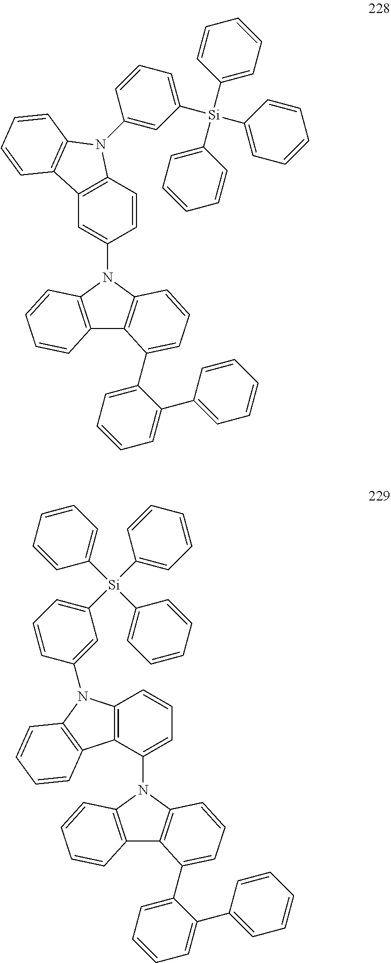 [SMILES: c1ccc(-c2ccccc2-c2cccc3c2c2ccccc2n3-c2ccc3c(c2)c2ccccc2n3-c2cccc([Si](c3ccccc3)(c3ccccc3)c3ccccc3)c2)cc1 ; c1ccc(-c2ccccc2-c2cccc3c2c2ccccc2n3-c2cccc3c2c2ccccc2n3-c2cccc([Si](c3ccccc3)(c3ccccc3)c3ccccc3)c2)cc1]

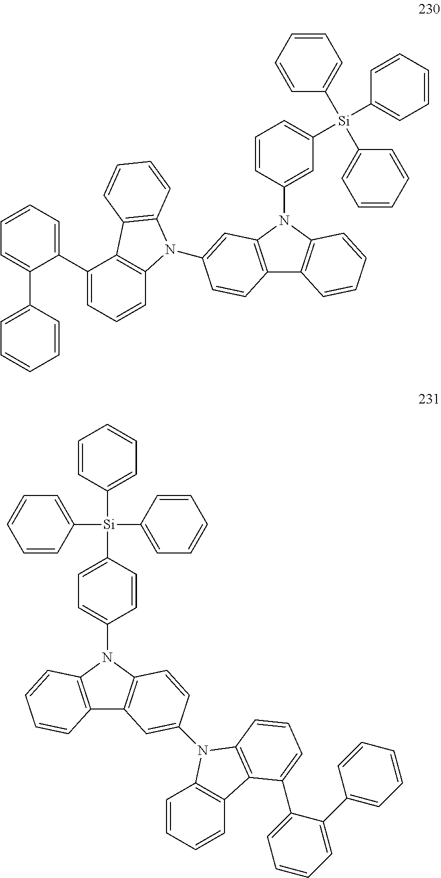 [SMILES: c1ccc(-c2ccccc2-c2cccc3c2c2ccccc2n3-c2ccc3c4ccccc4n(-c4cccc([Si](c5ccccc5)(c5ccccc5)c5ccccc5)c4)c3c2)cc1 ; c1ccc(-c2ccccc2-c2cccc3c2c2ccccc2n3-c2ccc3c(c2)c2ccccc2n3-c2ccc([Si](c3ccccc3)(c3ccccc3)c3ccccc3)cc2)cc1]

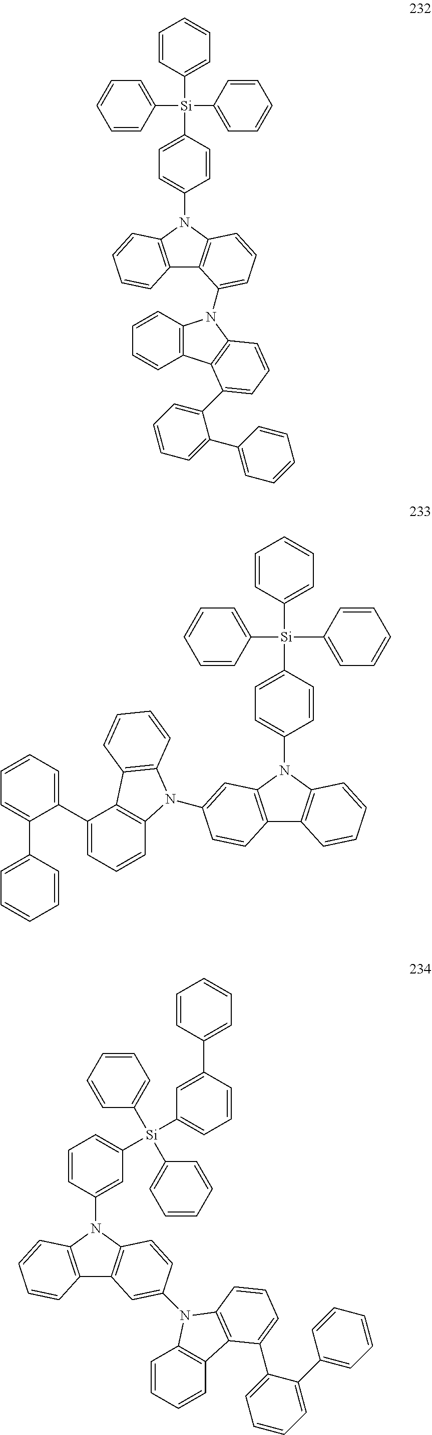 [SMILES: c1ccc(-c2ccccc2-c2cccc3c2c2ccccc2n3-c2cccc3c2c2ccccc2n3-c2ccc([Si](c3ccccc3)(c3ccccc3)c3ccccc3)cc2)cc1 ; c1ccc(-c2ccccc2-c2cccc3c2c2ccccc2n3-c2ccc3c4ccccc4n(-c4ccc([Si](c5ccccc5)(c5ccccc5)c5ccccc5)cc4)c3c2)cc1 ; c1ccc(-c2cccc([Si](c3ccccc3)(c3ccccc3)c3cccc(-n4c5ccccc5c5cc(-n6c7ccccc7c7c(-c8ccccc8-c8ccccc8)cccc76)ccc54)c3)c2)cc1]

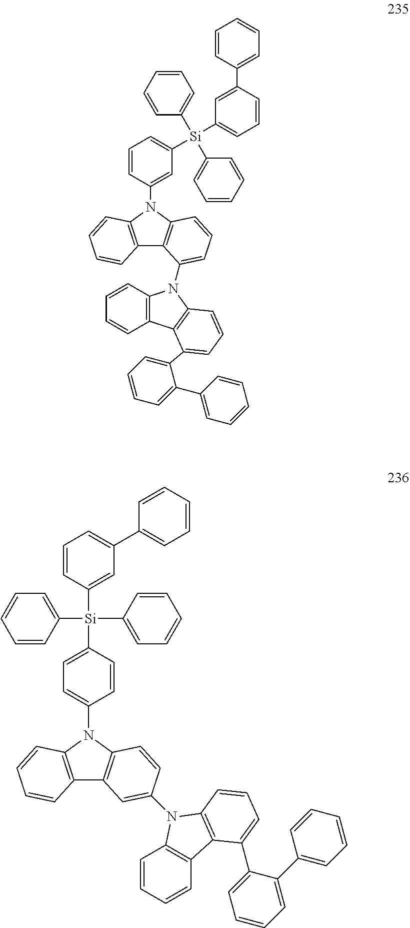 [SMILES: c1ccc(-c2cccc([Si](c3ccccc3)(c3ccccc3)c3cccc(-n4c5ccccc5c5c(-n6c7ccccc7c7c(-c8ccccc8-c8ccccc8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c(-c8ccccc8-c8ccccc8)cccc76)ccc54)cc3)c2)cc1]

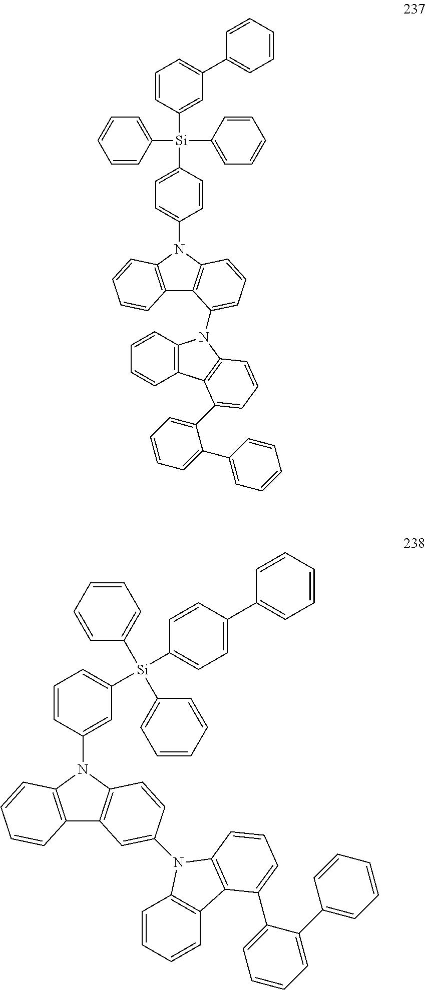 [SMILES: c1ccc(-c2cccc([Si](c3ccccc3)(c3ccccc3)c3ccc(-n4c5ccccc5c5c(-n6c7ccccc7c7c(-c8ccccc8-c8ccccc8)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c(-c8ccccc8-c8ccccc8)cccc76)ccc54)c3)cc2)cc1]

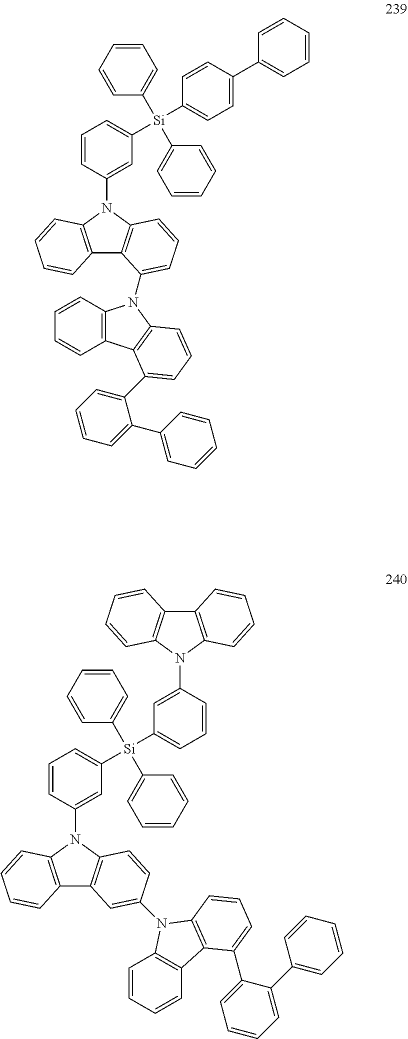 [SMILES: c1ccc(-c2ccc([Si](c3ccccc3)(c3ccccc3)c3cccc(-n4c5ccccc5c5c(-n6c7ccccc7c7c(-c8ccccc8-c8ccccc8)cccc76)cccc54)c3)cc2)cc1 ; c1ccc(-c2ccccc2-c2cccc3c2c2ccccc2n3-c2ccc3c(c2)c2ccccc2n3-c2cccc([Si](c3ccccc3)(c3ccccc3)c3cccc(-n4c5ccccc5c5ccccc54)c3)c2)cc1]

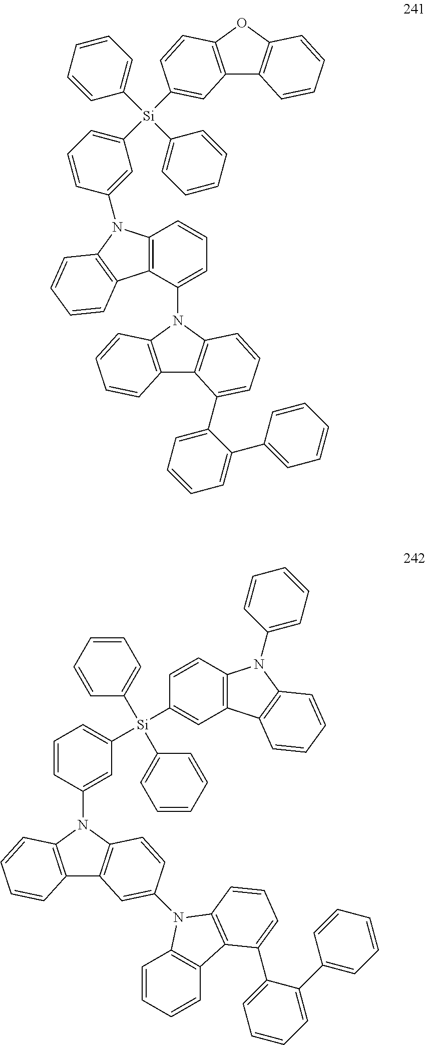 [SMILES: c1ccc(-c2ccccc2-c2cccc3c2c2ccccc2n3-c2cccc3c2c2ccccc2n3-c2cccc([Si](c3ccccc3)(c3ccccc3)c3ccc4oc5ccccc5c4c3)c2)cc1 ; c1ccc(-c2ccccc2-c2cccc3c2c2ccccc2n3-c2ccc3c(c2)c2ccccc2n3-c2cccc([Si](c3ccccc3)(c3ccccc3)c3ccc4c(c3)c3ccccc3n4-c3ccccc3)c2)cc1]

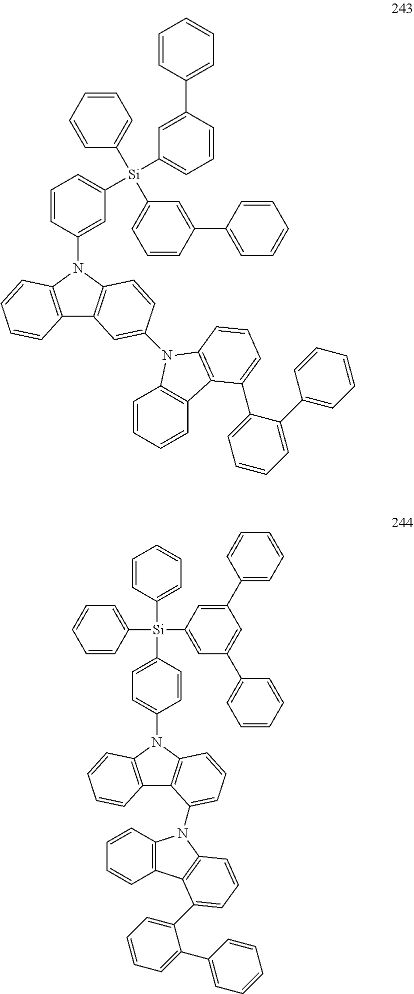 [SMILES: c1ccc(-c2cccc([Si](c3ccccc3)(c3cccc(-c4ccccc4)c3)c3cccc(-n4c5ccccc5c5cc(-n6c7ccccc7c7c(-c8ccccc8-c8ccccc8)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c(-c8ccccc8-c8ccccc8)cccc76)cccc54)cc3)c2)cc1]

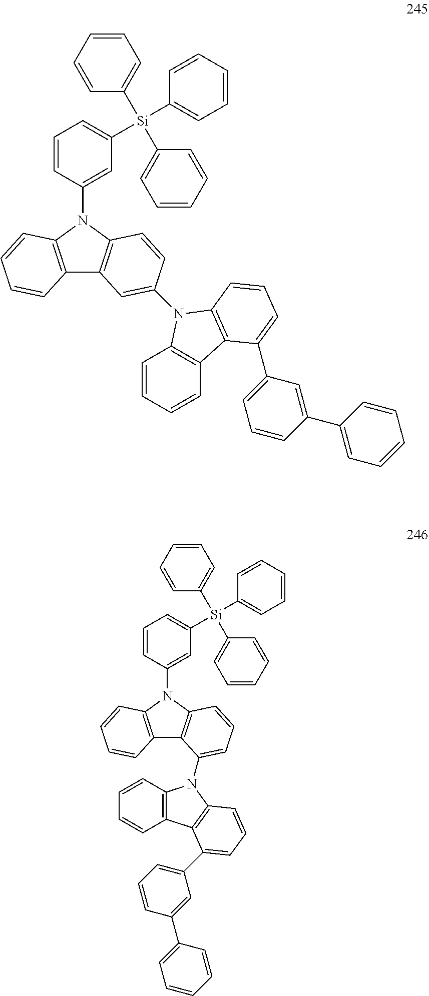 [SMILES: c1ccc(-c2cccc(-c3cccc4c3c3ccccc3n4-c3ccc4c(c3)c3ccccc3n4-c3cccc([Si](c4ccccc4)(c4ccccc4)c4ccccc4)c3)c2)cc1 ; c1ccc(-c2cccc(-c3cccc4c3c3ccccc3n4-c3cccc4c3c3ccccc3n4-c3cccc([Si](c4ccccc4)(c4ccccc4)c4ccccc4)c3)c2)cc1]

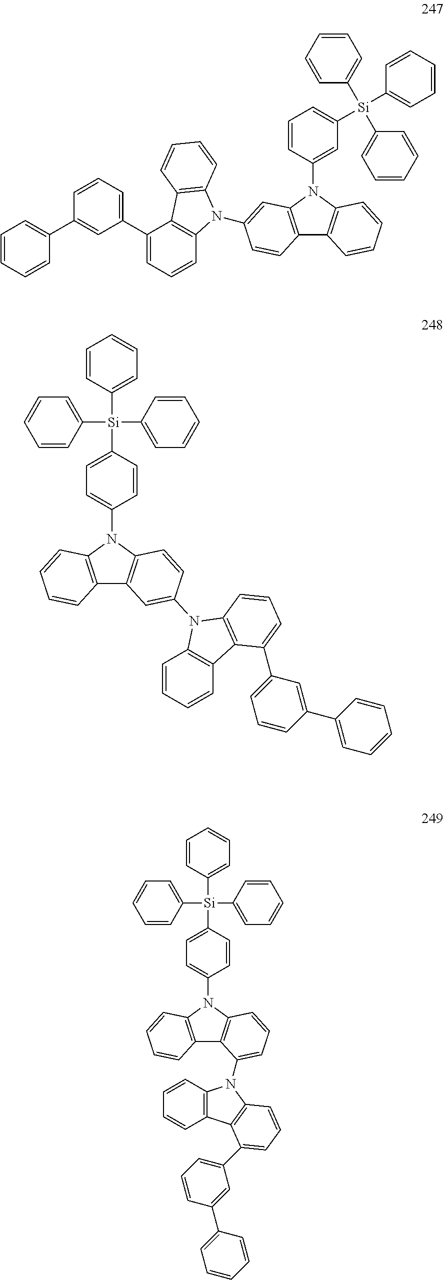 [SMILES: c1ccc(-c2cccc(-c3cccc4c3c3ccccc3n4-c3ccc4c5ccccc5n(-c5cccc([Si](c6ccccc6)(c6ccccc6)c6ccccc6)c5)c4c3)c2)cc1 ; c1ccc(-c2cccc(-c3cccc4c3c3ccccc3n4-c3ccc4c(c3)c3ccccc3n4-c3ccc([Si](c4ccccc4)(c4ccccc4)c4ccccc4)cc3)c2)cc1 ; c1ccc(-c2cccc(-c3cccc4c3c3ccccc3n4-c3cccc4c3c3ccccc3n4-c3ccc([Si](c4ccccc4)(c4ccccc4)c4ccccc4)cc3)c2)cc1]

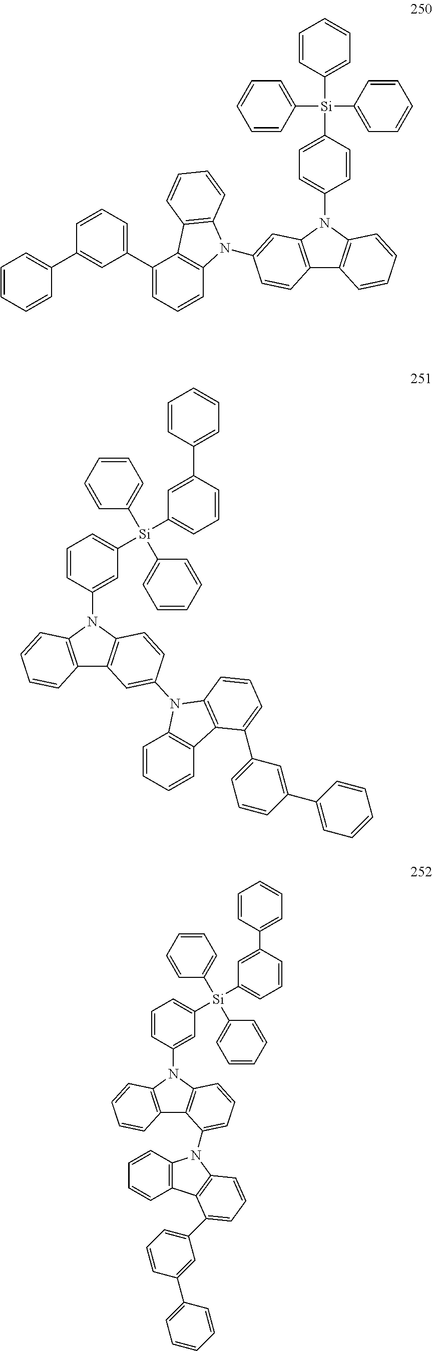 [SMILES: c1ccc(-c2cccc(-c3cccc4c3c3ccccc3n4-c3ccc4c5ccccc5n(-c5ccc([Si](c6ccccc6)(c6ccccc6)c6ccccc6)cc5)c4c3)c2)cc1 ; c1ccc(-c2cccc(-c3cccc4c3c3ccccc3n4-c3ccc4c(c3)c3ccccc3n4-c3cccc([Si](c4ccccc4)(c4ccccc4)c4cccc(-c5ccccc5)c4)c3)c2)cc1 ; c1ccc(-c2cccc(-c3cccc4c3c3ccccc3n4-c3cccc4c3c3ccccc3n4-c3cccc([Si](c4ccccc4)(c4ccccc4)c4cccc(-c5ccccc5)c4)c3)c2)cc1]

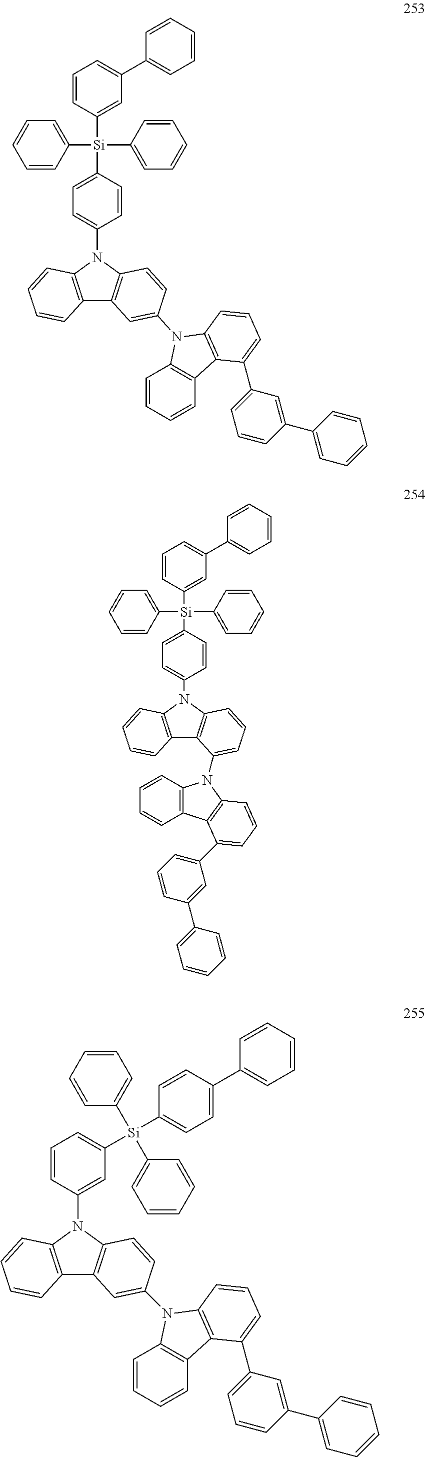 [SMILES: c1ccc(-c2cccc(-c3cccc4c3c3ccccc3n4-c3ccc4c(c3)c3ccccc3n4-c3ccc([Si](c4ccccc4)(c4ccccc4)c4cccc(-c5ccccc5)c4)cc3)c2)cc1 ; c1ccc(-c2cccc(-c3cccc4c3c3ccccc3n4-c3cccc4c3c3ccccc3n4-c3ccc([Si](c4ccccc4)(c4ccccc4)c4cccc(-c5ccccc5)c4)cc3)c2)cc1 ; c1ccc(-c2ccc([Si](c3ccccc3)(c3ccccc3)c3cccc(-n4c5ccccc5c5cc(-n6c7ccccc7c7c(-c8cccc(-c9ccccc9)c8)cccc76)ccc54)c3)cc2)cc1]

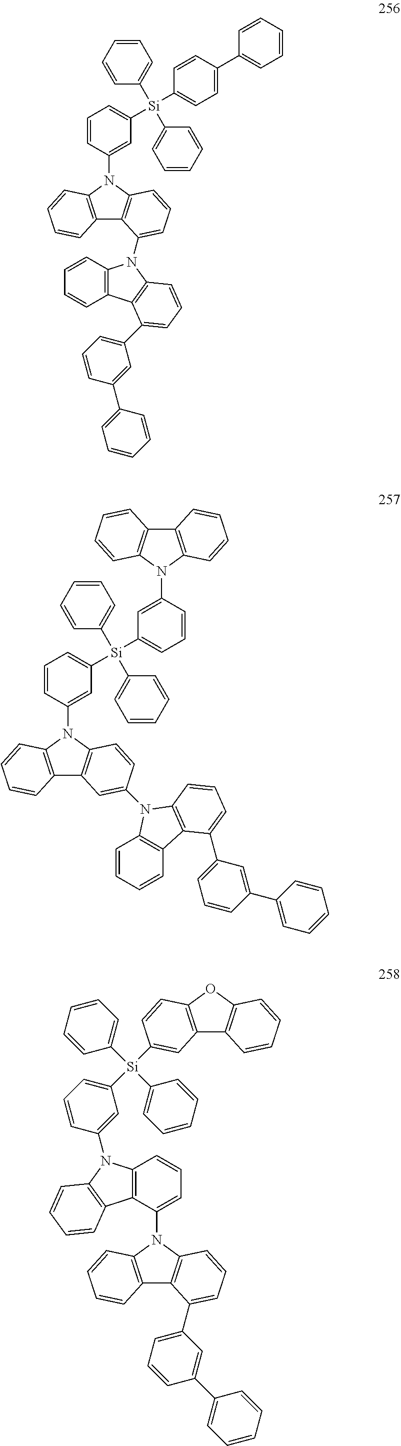 [SMILES: c1ccc(-c2ccc([Si](c3ccccc3)(c3ccccc3)c3cccc(-n4c5ccccc5c5c(-n6c7ccccc7c7c(-c8cccc(-c9ccccc9)c8)cccc76)cccc54)c3)cc2)cc1 ; c1ccc(-c2cccc(-c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)c2)cc1 ; c1ccc(-c2cccc(-c3cccc4c3c3ccccc3n4-c3cccc4c3c3ccccc3n4-c3cccc([Si](c4ccccc4)(c4ccccc4)c4ccc5oc6ccccc6c5c4)c3)c2)cc1]

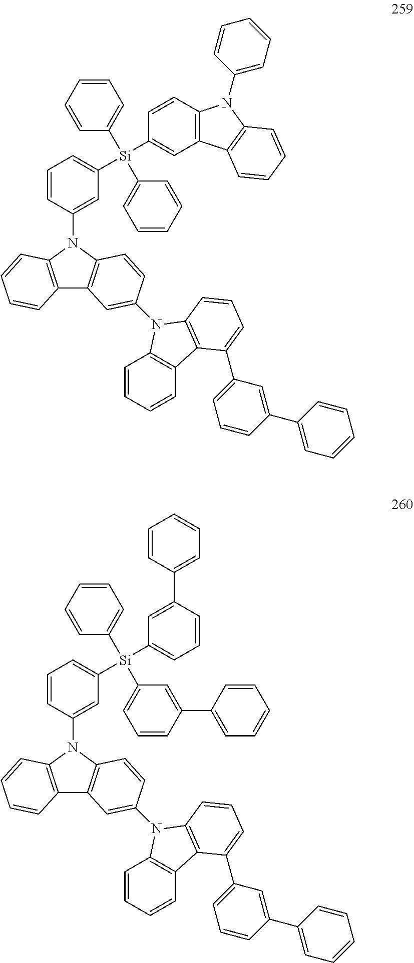 [SMILES: c1ccc(-c2cccc(-c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)c2)cc1 ; c1ccc(-c2cccc(-c3cccc4c3c3ccccc3n4-c3ccc4c(c3)c3ccccc3n4-c3cccc([Si](c4ccccc4)(c4cccc(-c5ccccc5)c4)c4cccc(-c5ccccc5)c4)c3)c2)cc1]

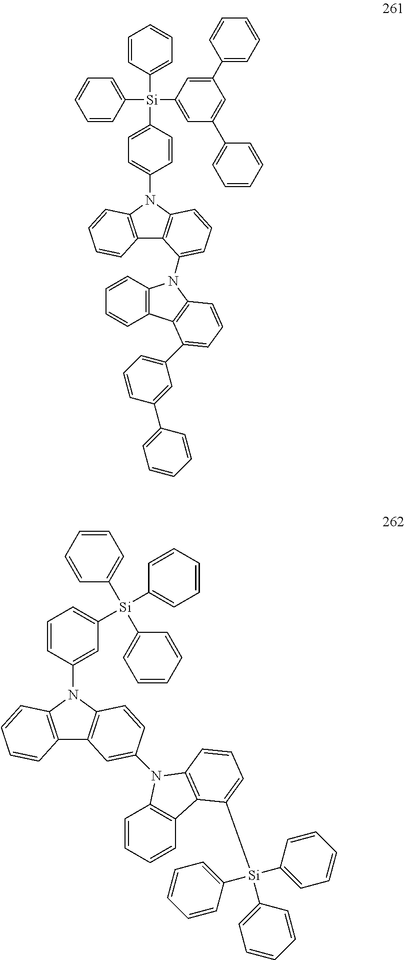 [SMILES: c1ccc(-c2cccc(-c3cccc4c3c3ccccc3n4-c3cccc4c3c3ccccc3n4-c3ccc([Si](c4ccccc4)(c4ccccc4)c4cc(-c5ccccc5)cc(-c5ccccc5)c4)cc3)c2)cc1 ; c1ccc([Si](c2ccccc2)(c2ccccc2)c2cccc(-n3c4ccccc4c4cc(-n5c6ccccc6c6c([Si](c7ccccc7)(c7ccccc7)c7ccccc7)cccc65)ccc43)c2)cc1]

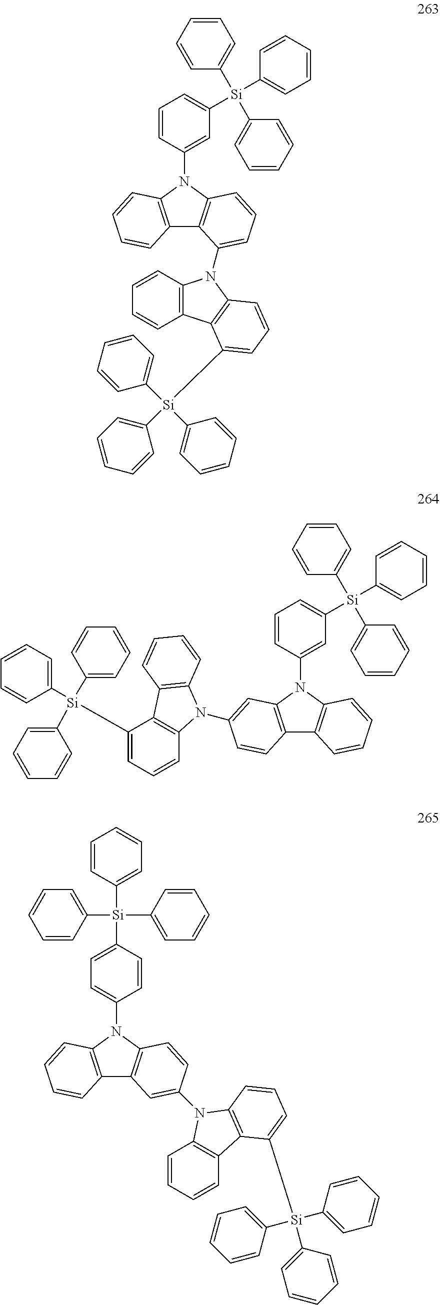 [SMILES: c1ccc([Si](c2ccccc2)(c2ccccc2)c2cccc(-n3c4ccccc4c4c(-n5c6ccccc6c6c([Si](c7ccccc7)(c7ccccc7)c7ccccc7)cccc65)cccc43)c2)cc1 ; c1ccc([Si](c2ccccc2)(c2ccccc2)c2cccc(-n3c4ccccc4c4ccc(-n5c6ccccc6c6c([Si](c7ccccc7)(c7ccccc7)c7ccccc7)cccc65)cc43)c2)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7ccccc7)cccc65)ccc43)cc2)cc1]

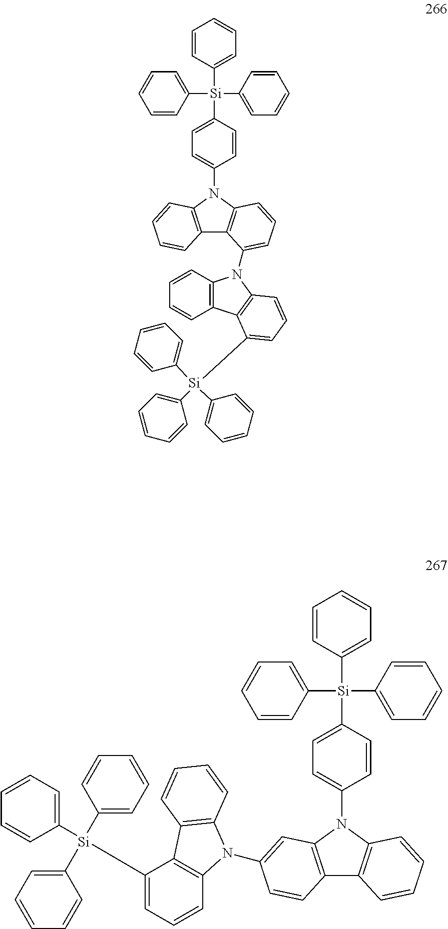 [SMILES: c1ccc([Si](c2ccccc2)(c2ccccc2)c2ccc(-n3c4ccccc4c4c(-n5c6ccccc6c6c([Si](c7ccccc7)(c7ccccc7)c7ccccc7)cccc65)cccc43)cc2)cc1 ; c1ccc([Si](c2ccccc2)(c2ccccc2)c2ccc(-n3c4ccccc4c4ccc(-n5c6ccccc6c6c([Si](c7ccccc7)(c7ccccc7)c7ccccc7)cccc65)cc43)cc2)cc1]

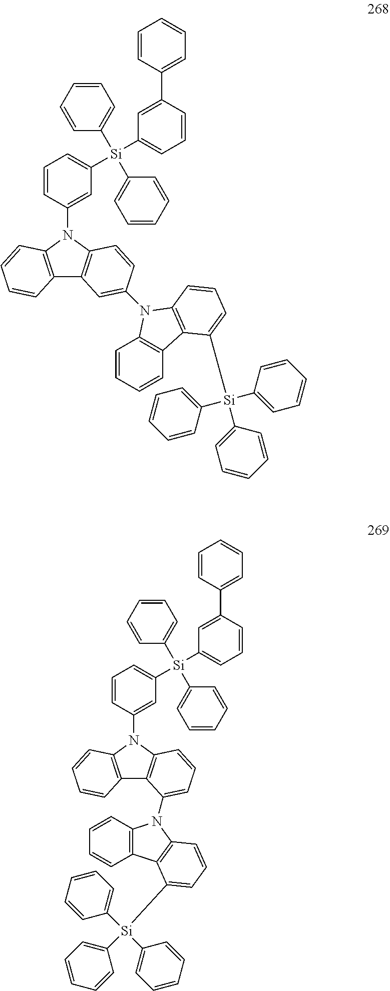 [SMILES: c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8ccccc8)cccc76)ccc54)c3)c2)cc1 ; c1ccc(-c2cccc([Si](c3ccccc3)(c3ccccc3)c3cccc(-n4c5ccccc5c5c(-n6c7ccccc7c7c([Si](c8ccccc8)(c8ccccc8)c8ccccc8)cccc76)cccc54)c3)c2)cc1]

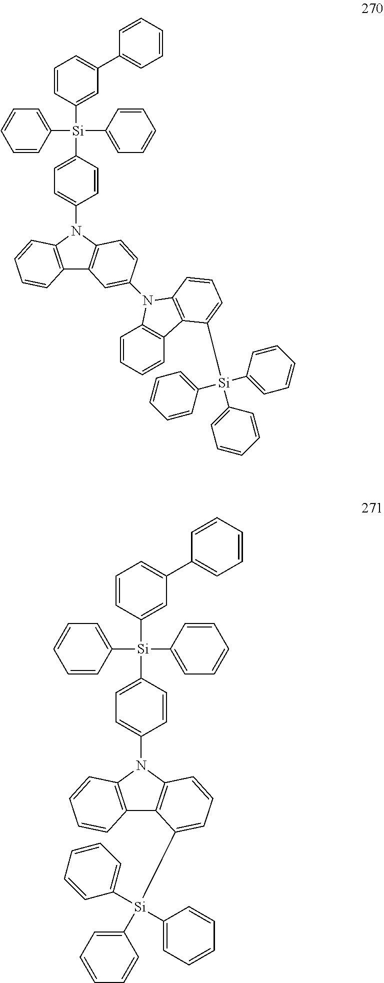 [SMILES: c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8ccccc8)cccc76)ccc54)cc3)c2)cc1 ; c1ccc(-c2cccc([Si](c3ccccc3)(c3ccccc3)c3ccc(-n4c5ccccc5c5c([Si](c6ccccc6)(c6ccccc6)c6ccccc6)cccc54)cc3)c2)cc1]

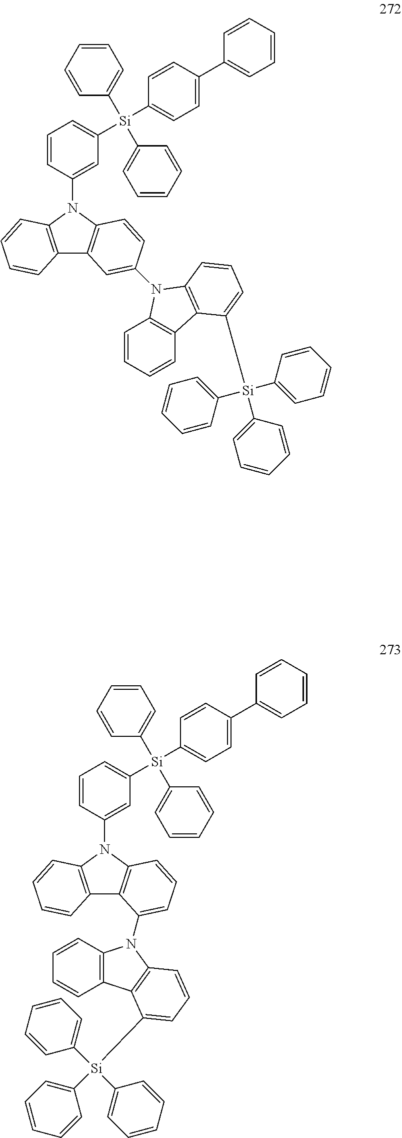 [SMILES: c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccccc8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8ccccc8)cccc76)cccc54)c3)cc2)cc1]

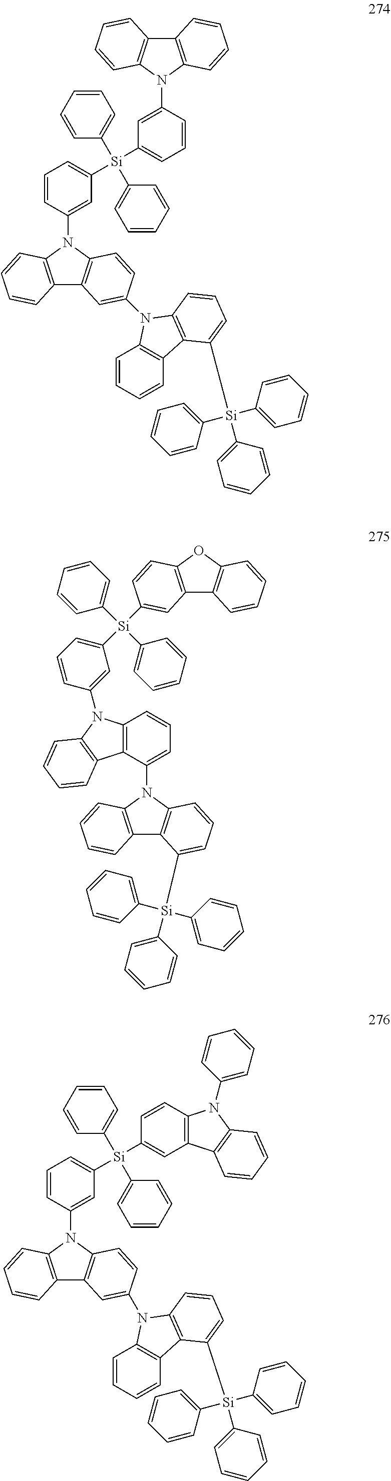 [SMILES: c1ccc([Si](c2ccccc2)(c2cccc(-n3c4ccccc4c4ccccc43)c2)c2cccc(-n3c4ccccc4c4cc(-n5c6ccccc6c6c([Si](c7ccccc7)(c7ccccc7)c7ccccc7)cccc65)ccc43)c2)cc1 ; c1ccc([Si](c2ccccc2)(c2cccc(-n3c4ccccc4c4c(-n5c6ccccc6c6c([Si](c7ccccc7)(c7ccccc7)c7ccccc7)cccc65)cccc43)c2)c2ccc3oc4ccccc4c3c2)cc1 ; c1ccc(-n2c3ccccc3c3cc([Si](c4ccccc4)(c4ccccc4)c4cccc(-n5c6ccccc6c6cc(-n7c8ccccc8c8c([Si](c9ccccc9)(c9ccccc9)c9ccccc9)cccc87)ccc65)c4)ccc32)cc1]

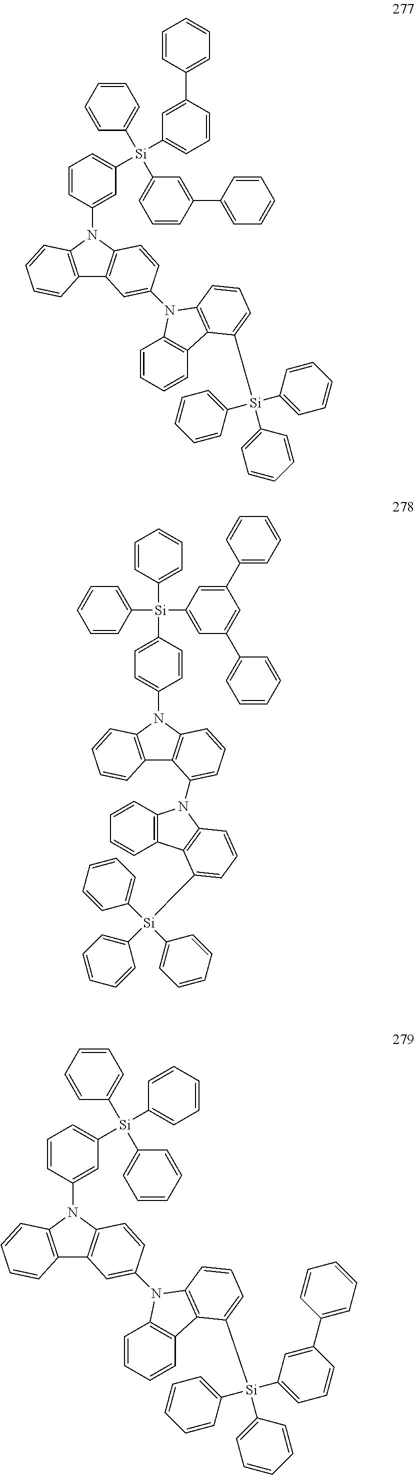

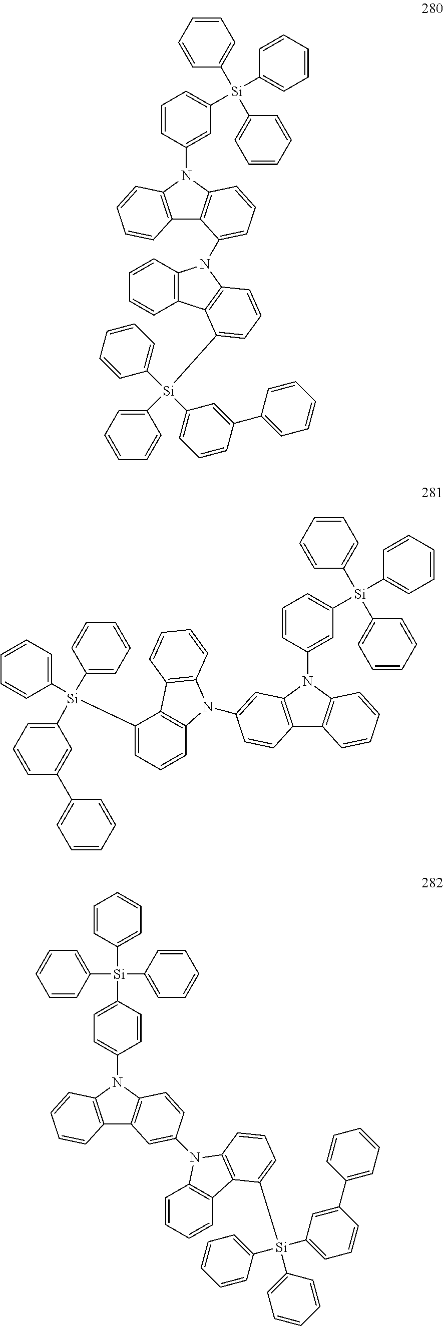 [SMILES: c1ccc(-c2cccc([Si](c3ccccc3)(c3ccccc3)c3cccc4c3c3ccccc3n4-c3cccc4c3c3ccccc3n4-c3cccc([Si](c4ccccc4)(c4ccccc4)c4ccccc4)c3)c2)cc1 ; c1ccc(-c2cccc([Si](c3ccccc3)(c3ccccc3)c3cccc4c3c3ccccc3n4-c3ccc4c5ccccc5n(-c5cccc([Si](c6ccccc6)(c6ccccc6)c6ccccc6)c5)c4c3)c2)cc1 ; c1ccc(-c2cccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4ccccc4)cc3)c2)cc1]

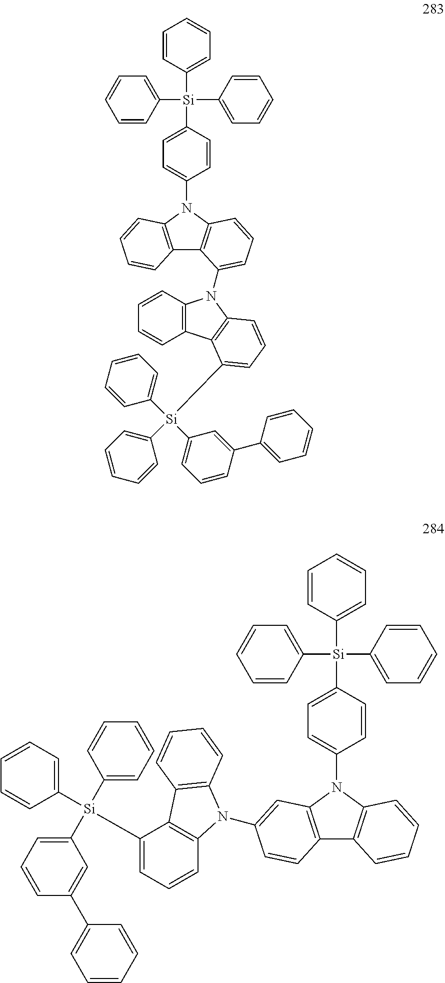 [SMILES: c1ccc(-c2cccc([Si](c3ccccc3)(c3ccccc3)c3cccc4c3c3ccccc3n4-c3cccc4c3c3ccccc3n4-c3ccc([Si](c4ccccc4)(c4ccccc4)c4ccccc4)cc3)c2)cc1 ; c1ccc(-c2cccc([Si](c3ccccc3)(c3ccccc3)c3cccc4c3c3ccccc3n4-c3ccc4c5ccccc5n(-c5ccc([Si](c6ccccc6)(c6ccccc6)c6ccccc6)cc5)c4c3)c2)cc1]

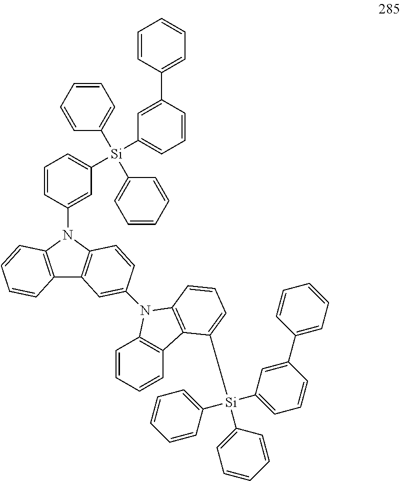 [SMILES: c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1]

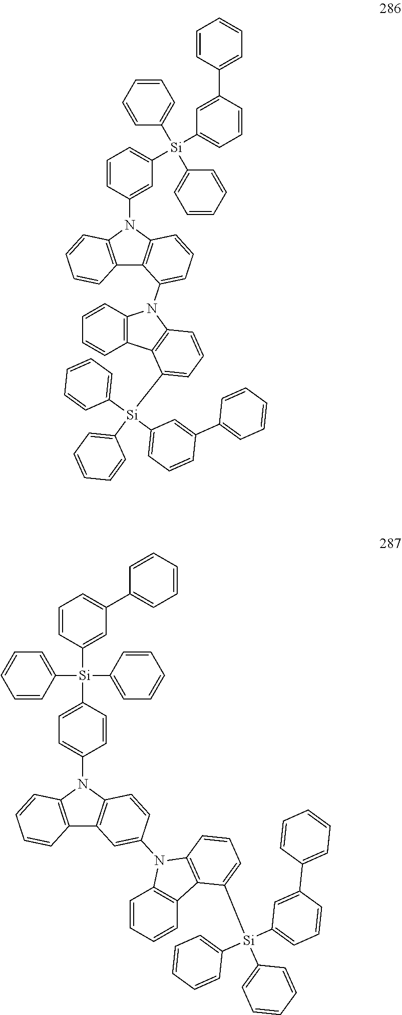 [SMILES: c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc(-c9ccccc9)c8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8cccc(-c9ccccc9)c8)cccc76)ccc54)cc3)c2)cc1]

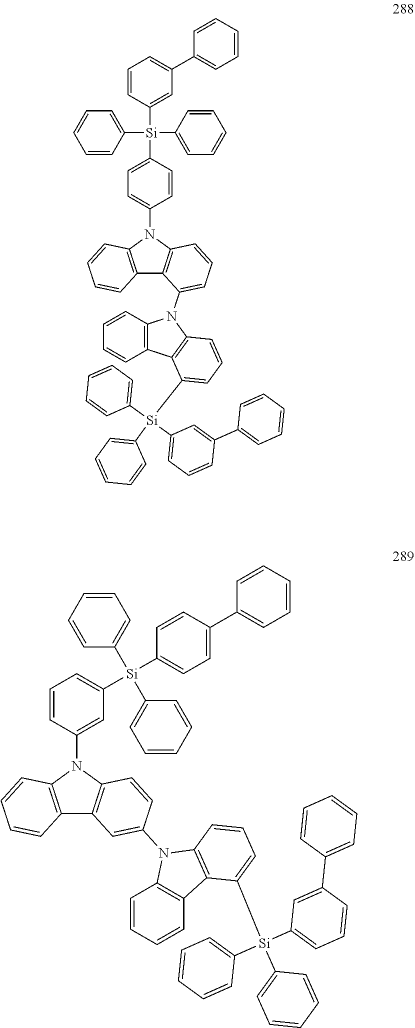 [SMILES: c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc(-c9ccccc9)c8)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8cccc(-c9ccccc9)c8)cccc76)ccc54)c3)cc2)cc1]

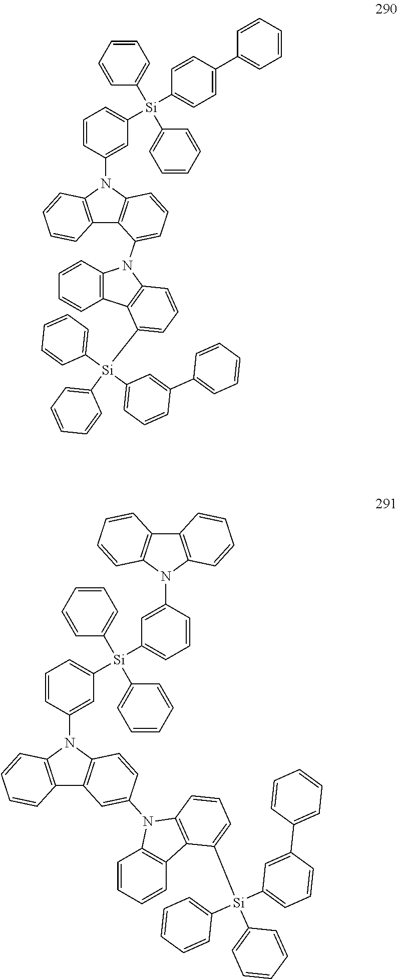 [SMILES: c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc(-c9ccccc9)c8)cccc76)cccc54)c3)cc2)cc1 ; c1ccc(-c2cccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)c2)cc1]

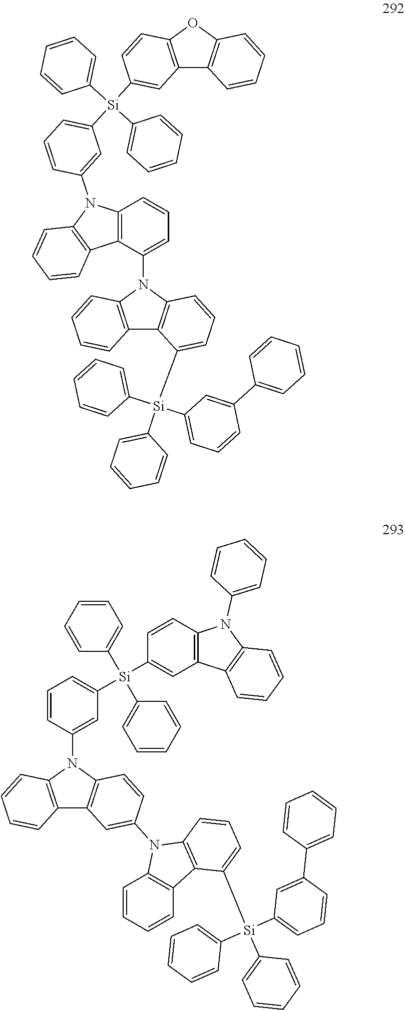 [SMILES: c1ccc(-c2cccc([Si](c3ccccc3)(c3ccccc3)c3cccc4c3c3ccccc3n4-c3cccc4c3c3ccccc3n4-c3cccc([Si](c4ccccc4)(c4ccccc4)c4ccc5oc6ccccc6c5c4)c3)c2)cc1 ; c1ccc(-c2cccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)c2)cc1]

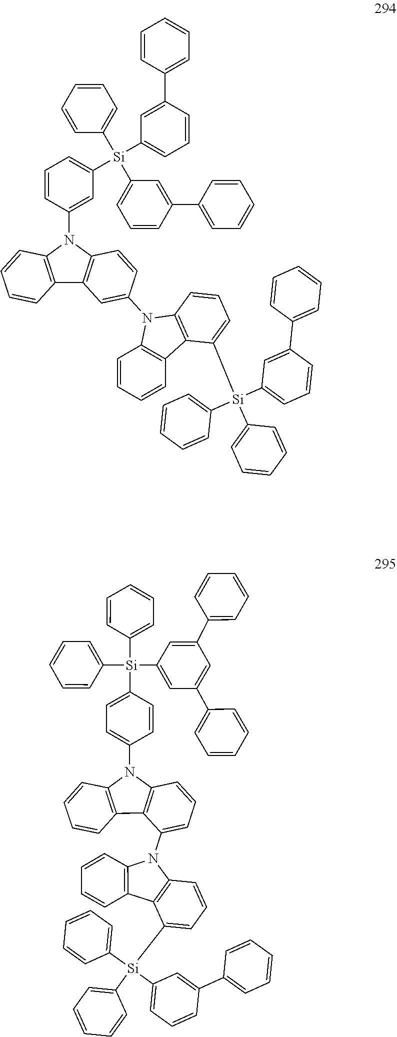 [SMILES: c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8cccc(-c9ccccc9)c8)cccc76)cccc54)cc3)c2)cc1]

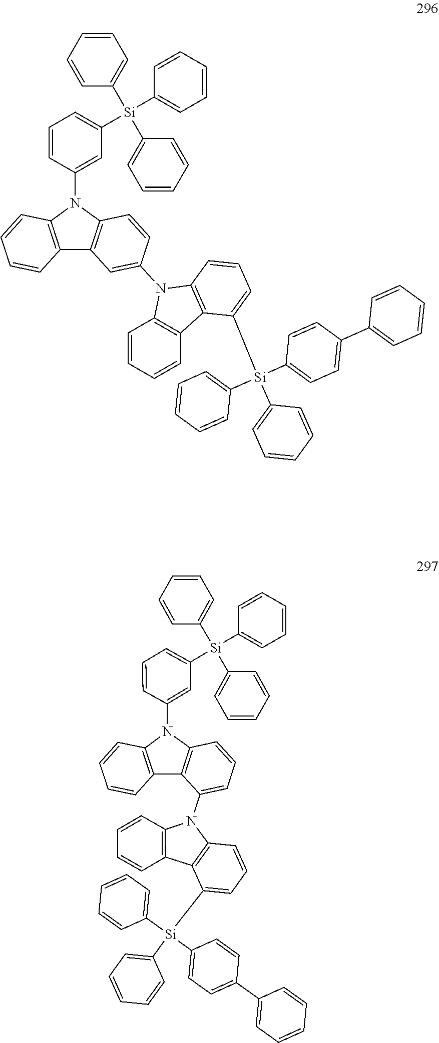 [SMILES: c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccccc4)c3)cc2)cc1 ; c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4ccccc4)c3)cc2)cc1]

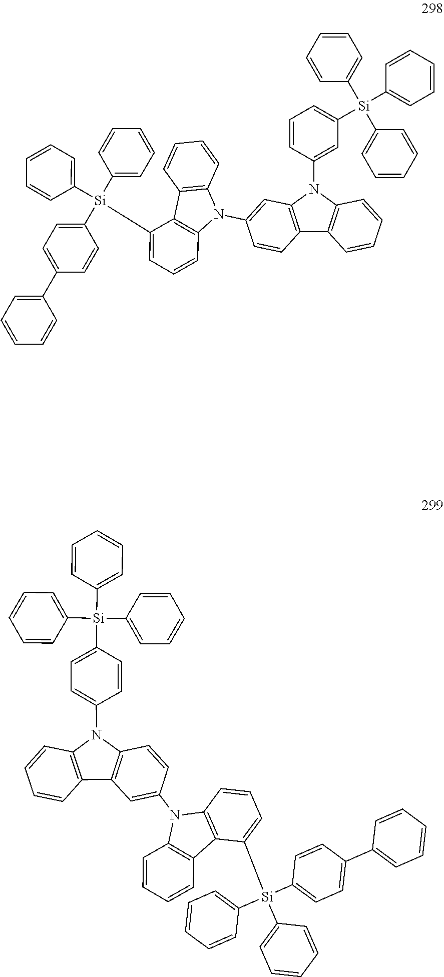 [SMILES: c1ccc(-c2ccc([Si](c3ccccc3)(c3ccccc3)c3cccc4c3c3ccccc3n4-c3ccc4c5ccccc5n(-c5cccc([Si](c6ccccc6)(c6ccccc6)c6ccccc6)c5)c4c3)cc2)cc1 ; c1ccc(-c2ccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4ccccc4)cc3)cc2)cc1]

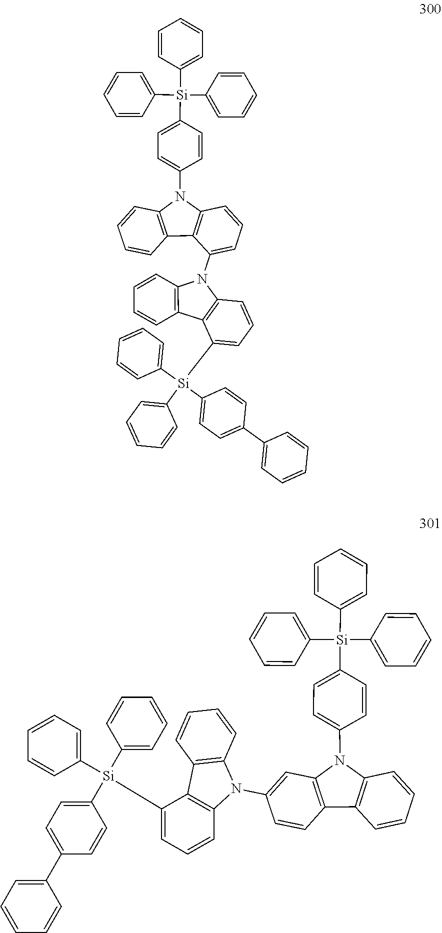 [SMILES: c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4ccccc4)cc3)cc2)cc1 ; c1ccc(-c2ccc([Si](c3ccccc3)(c3ccccc3)c3cccc4c3c3ccccc3n4-c3ccc4c5ccccc5n(-c5ccc([Si](c6ccccc6)(c6ccccc6)c6ccccc6)cc5)c4c3)cc2)cc1]

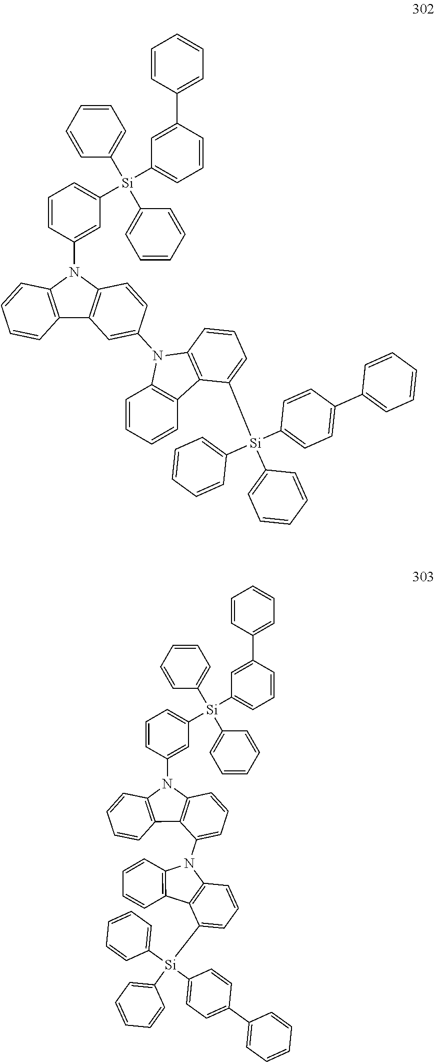 [SMILES: c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-c5ccccc5)c4)c3)cc2)cc1 ; c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4cccc(-c5ccccc5)c4)c3)cc2)cc1]

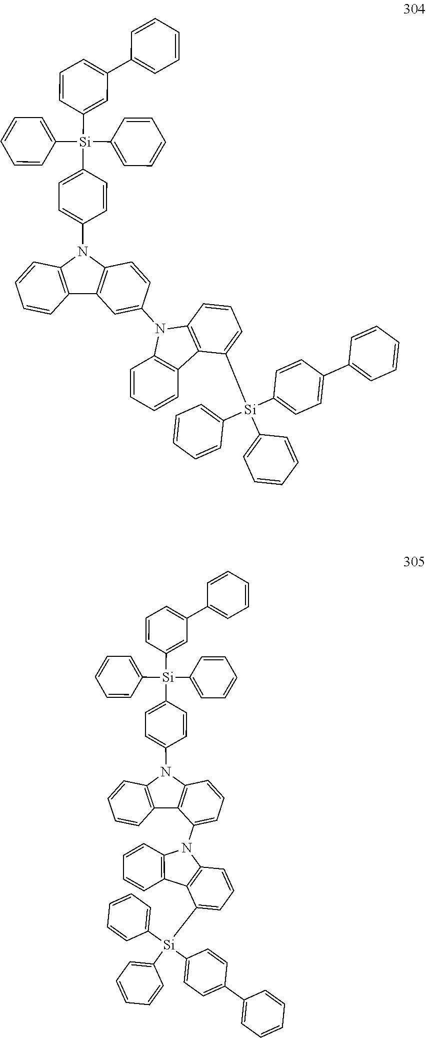 [SMILES: c1ccc(-c2ccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4cccc(-c5ccccc5)c4)cc3)cc2)cc1 ; c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4cccc(-c5ccccc5)c4)cc3)cc2)cc1]

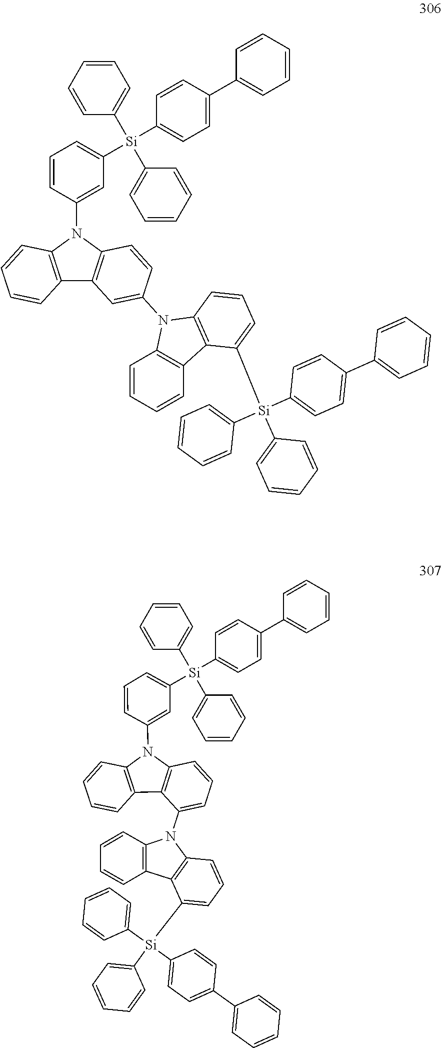 [SMILES: c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccc(-c9ccccc9)cc8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8ccc(-c9ccccc9)cc8)cccc76)cccc54)c3)cc2)cc1]

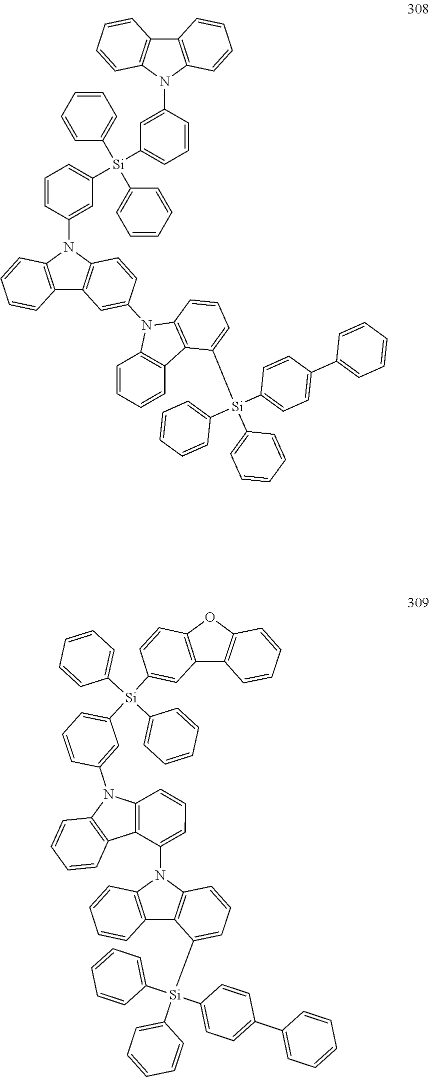 [SMILES: c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)cc2)cc1 ; c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4ccc5oc6ccccc6c5c4)c3)cc2)cc1]

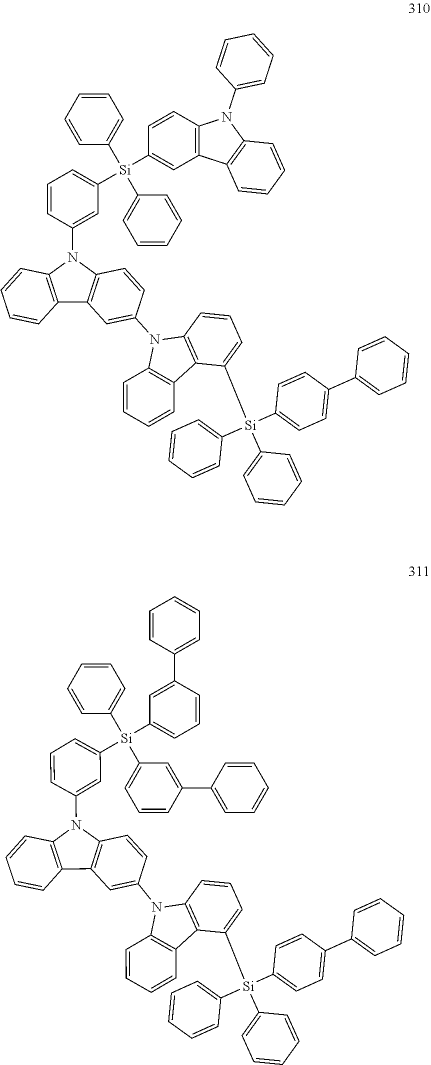 [SMILES: c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4cccc(-c5ccccc5)c4)c4cccc(-c5ccccc5)c4)c3)cc2)cc1]

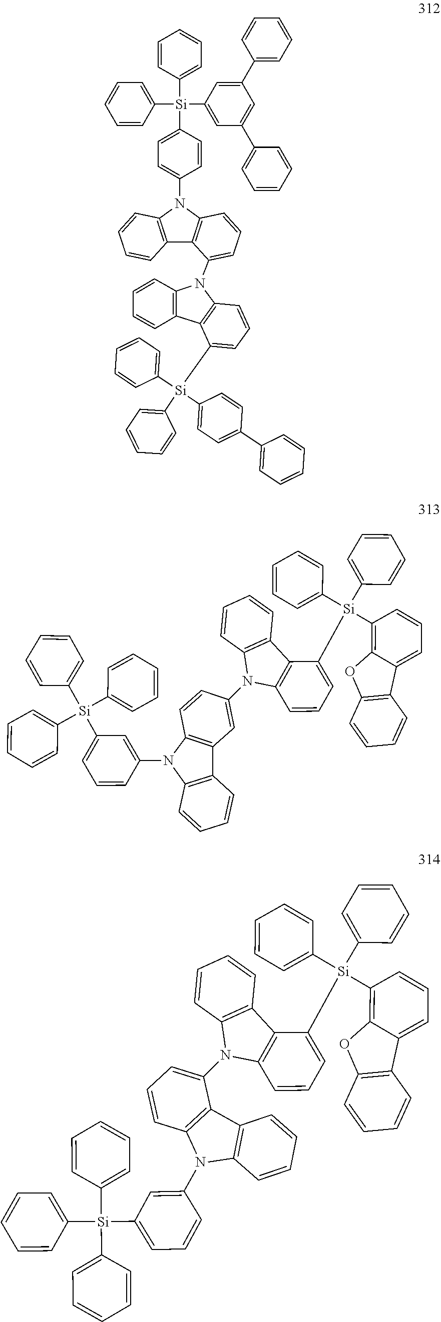 [SMILES: c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4cc(-c5ccccc5)cc(-c5ccccc5)c4)cc3)cc2)cc1 ; c1ccc([Si](c2ccccc2)(c2ccccc2)c2cccc(-n3c4ccccc4c4cc(-n5c6ccccc6c6c([Si](c7ccccc7)(c7ccccc7)c7cccc8c7oc7ccccc78)cccc65)ccc43)c2)cc1 ; c1ccc([Si](c2ccccc2)(c2ccccc2)c2cccc(-n3c4ccccc4c4c(-n5c6ccccc6c6c([Si](c7ccccc7)(c7ccccc7)c7cccc8c7oc7ccccc78)cccc65)cccc43)c2)cc1]

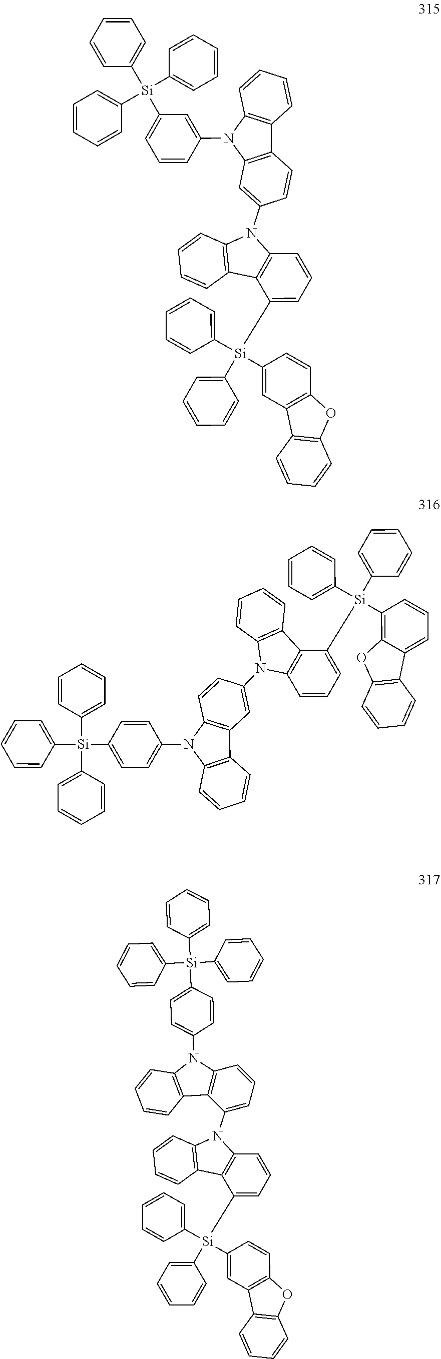 [SMILES: c1ccc([Si](c2ccccc2)(c2ccccc2)c2cccc(-n3c4ccccc4c4ccc(-n5c6ccccc6c6c([Si](c7ccccc7)(c7ccccc7)c7ccc8oc9ccccc9c8c7)cccc65)cc43)c2)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7cccc8c7oc7ccccc78)cccc65)ccc43)cc2)cc1 ; c1ccc([Si](c2ccccc2)(c2ccccc2)c2ccc(-n3c4ccccc4c4c(-n5c6ccccc6c6c([Si](c7ccccc7)(c7ccccc7)c7ccc8oc9ccccc9c8c7)cccc65)cccc43)cc2)cc1]

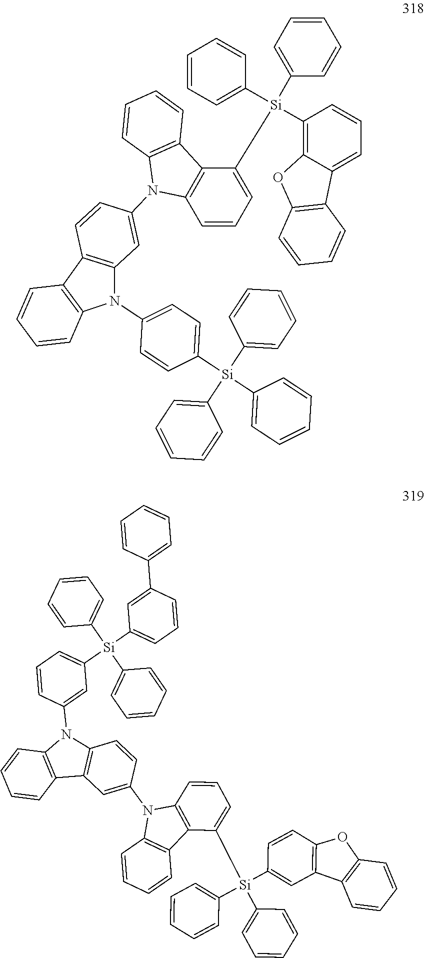 [SMILES: c1ccc([Si](c2ccccc2)(c2ccccc2)c2ccc(-n3c4ccccc4c4ccc(-n5c6ccccc6c6c([Si](c7ccccc7)(c7ccccc7)c7cccc8c7oc7ccccc78)cccc65)cc43)cc2)cc1 ; c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8ccc9oc%10ccccc%10c9c8)cccc76)ccc54)c3)c2)cc1]

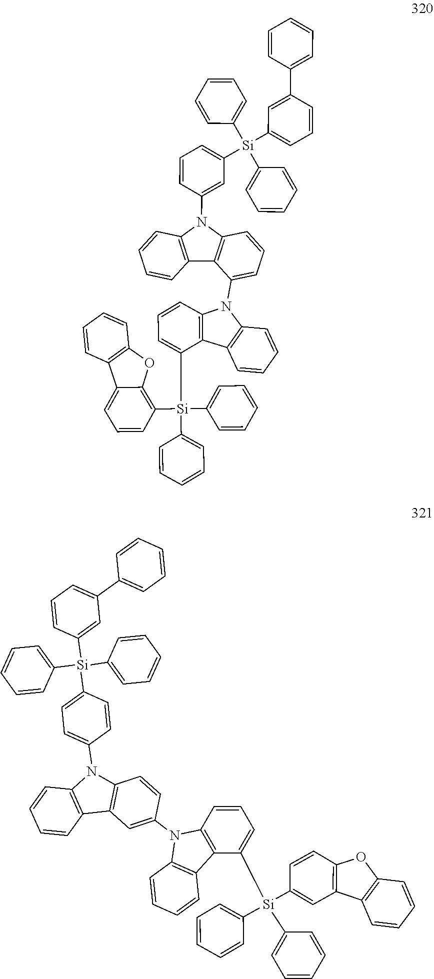 [SMILES: c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc9c8oc8ccccc89)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8ccc9oc%10ccccc%10c9c8)cccc76)ccc54)cc3)c2)cc1]

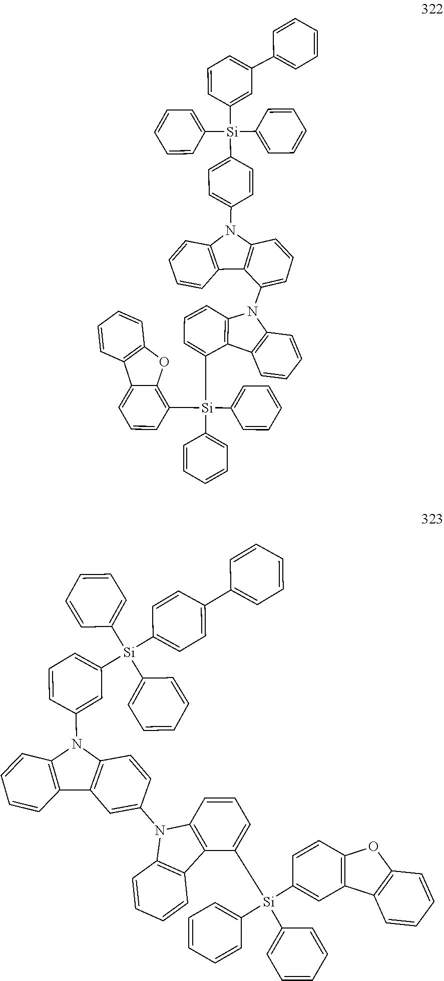 [SMILES: c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc9c8oc8ccccc89)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccc9oc%10ccccc%10c9c8)cccc76)ccc54)c3)cc2)cc1]

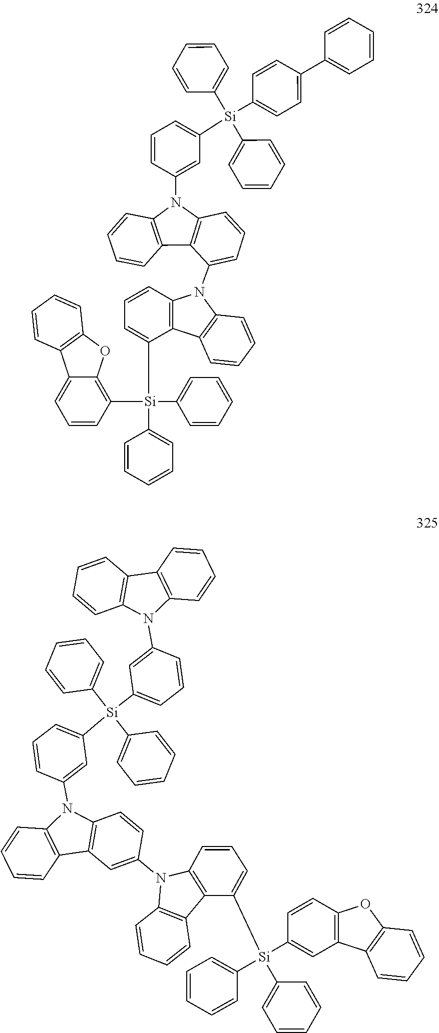 [SMILES: c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc9c8oc8ccccc89)cccc76)cccc54)c3)cc2)cc1 ; c1ccc([Si](c2ccccc2)(c2cccc(-n3c4ccccc4c4ccccc43)c2)c2cccc(-n3c4ccccc4c4cc(-n5c6ccccc6c6c([Si](c7ccccc7)(c7ccccc7)c7ccc8oc9ccccc9c8c7)cccc65)ccc43)c2)cc1]

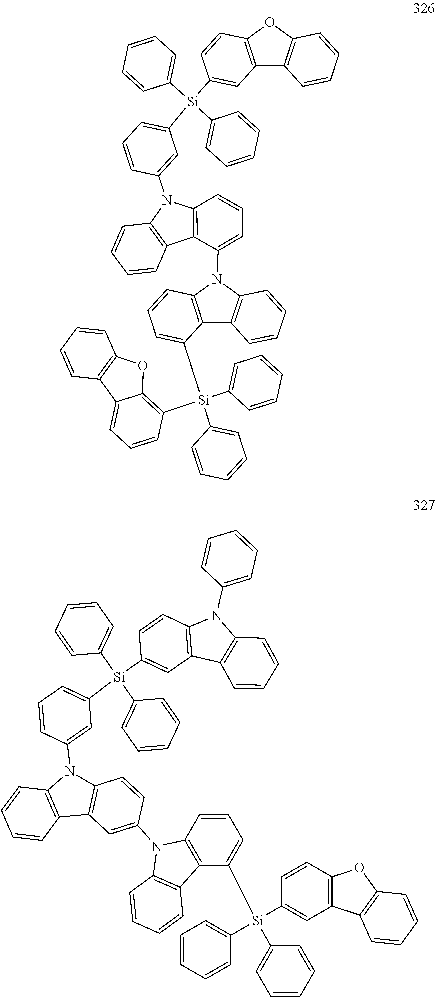 [SMILES: c1ccc([Si](c2ccccc2)(c2cccc(-n3c4ccccc4c4c(-n5c6ccccc6c6c([Si](c7ccccc7)(c7ccccc7)c7cccc8c7oc7ccccc78)cccc65)cccc43)c2)c2ccc3oc4ccccc4c3c2)cc1 ; c1ccc(-n2c3ccccc3c3cc([Si](c4ccccc4)(c4ccccc4)c4cccc(-n5c6ccccc6c6cc(-n7c8ccccc8c8c([Si](c9ccccc9)(c9ccccc9)c9ccc%10oc%11ccccc%11c%10c9)cccc87)ccc65)c4)ccc32)cc1]

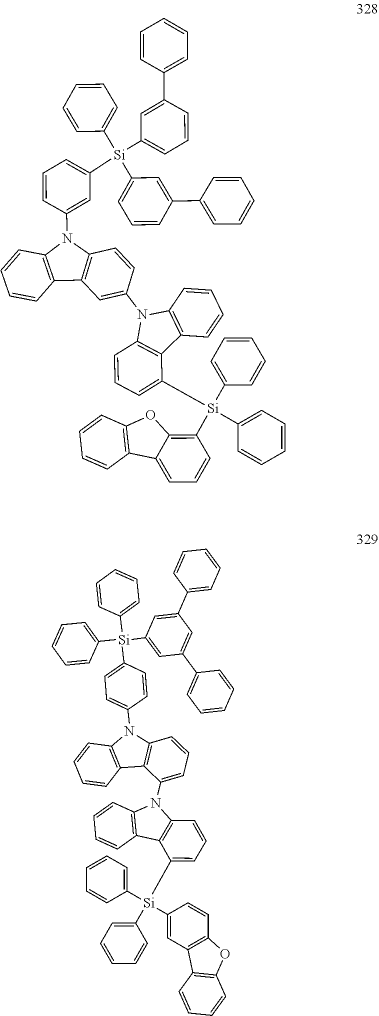 [SMILES: c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8cccc9c8oc8ccccc89)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8ccc9oc%10ccccc%10c9c8)cccc76)cccc54)cc3)c2)cc1]

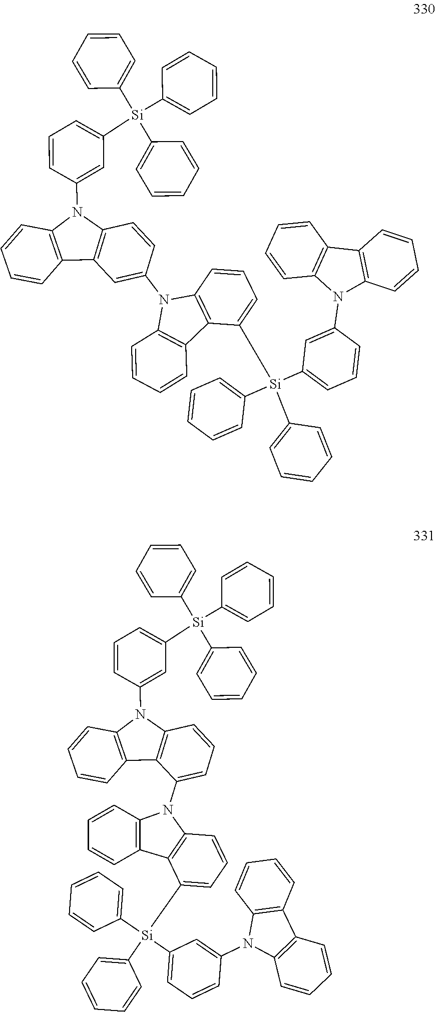 [SMILES: c1ccc([Si](c2ccccc2)(c2ccccc2)c2cccc(-n3c4ccccc4c4cc(-n5c6ccccc6c6c([Si](c7ccccc7)(c7ccccc7)c7cccc(-n8c9ccccc9c9ccccc98)c7)cccc65)ccc43)c2)cc1 ; c1ccc([Si](c2ccccc2)(c2ccccc2)c2cccc(-n3c4ccccc4c4c(-n5c6ccccc6c6c([Si](c7ccccc7)(c7ccccc7)c7cccc(-n8c9ccccc9c9ccccc98)c7)cccc65)cccc43)c2)cc1]

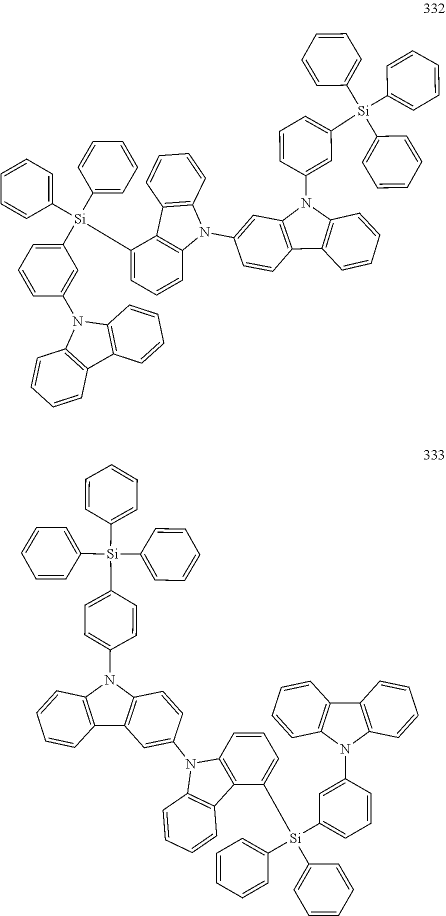 [SMILES: c1ccc([Si](c2ccccc2)(c2ccccc2)c2cccc(-n3c4ccccc4c4ccc(-n5c6ccccc6c6c([Si](c7ccccc7)(c7ccccc7)c7cccc(-n8c9ccccc9c9ccccc98)c7)cccc65)cc43)c2)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7cccc(-n8c9ccccc9c9ccccc98)c7)cccc65)ccc43)cc2)cc1]

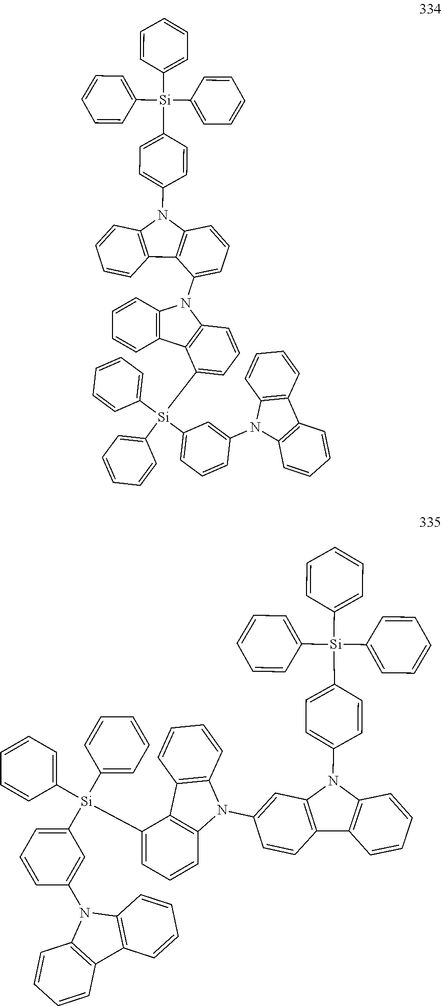 [SMILES: c1ccc([Si](c2ccccc2)(c2ccccc2)c2ccc(-n3c4ccccc4c4c(-n5c6ccccc6c6c([Si](c7ccccc7)(c7ccccc7)c7cccc(-n8c9ccccc9c9ccccc98)c7)cccc65)cccc43)cc2)cc1 ; c1ccc([Si](c2ccccc2)(c2ccccc2)c2ccc(-n3c4ccccc4c4ccc(-n5c6ccccc6c6c([Si](c7ccccc7)(c7ccccc7)c7cccc(-n8c9ccccc9c9ccccc98)c7)cccc65)cc43)cc2)cc1]

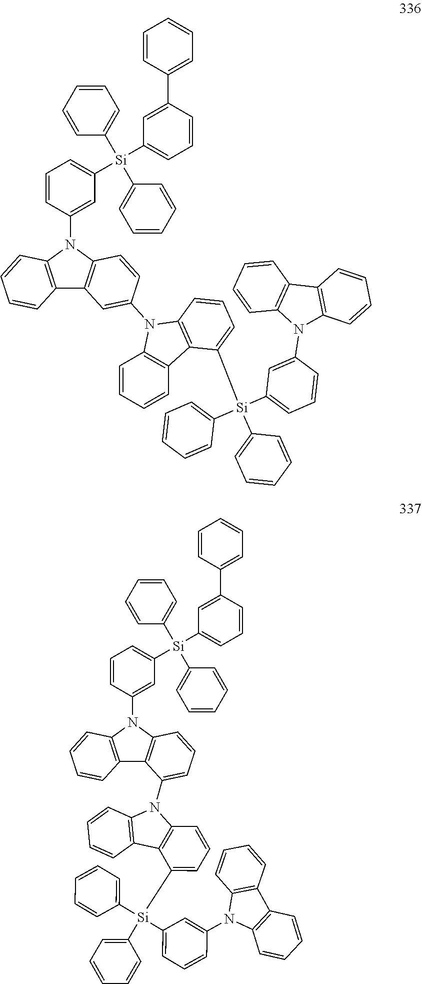 [SMILES: c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)c2)cc1 ; c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)c3)c2)cc1]

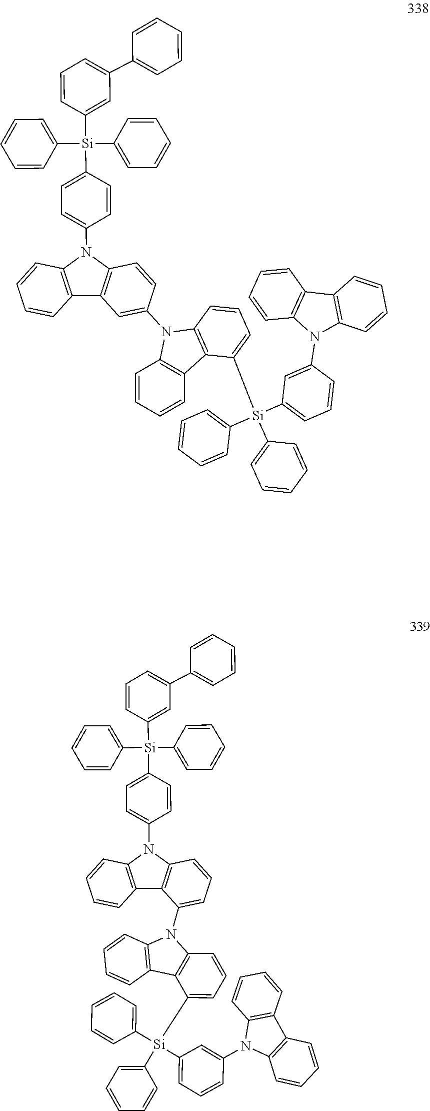 [SMILES: c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)cc3)c2)cc1 ; c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)cc3)c2)cc1]

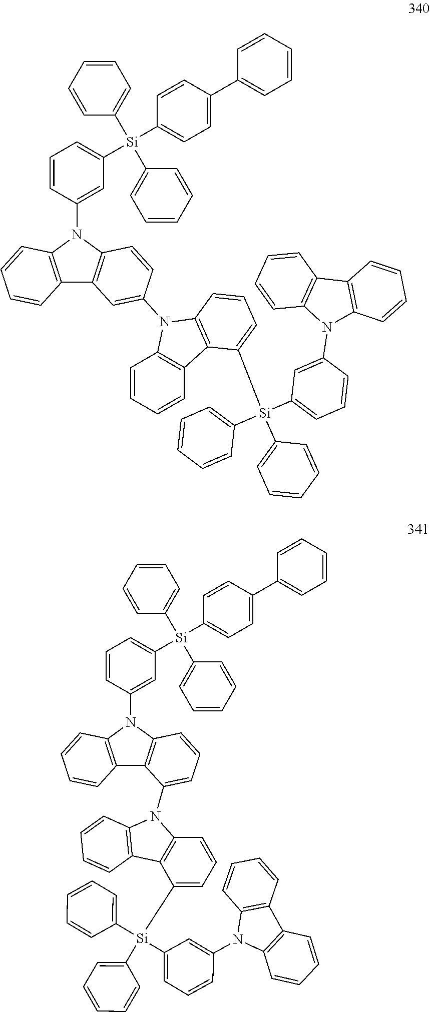

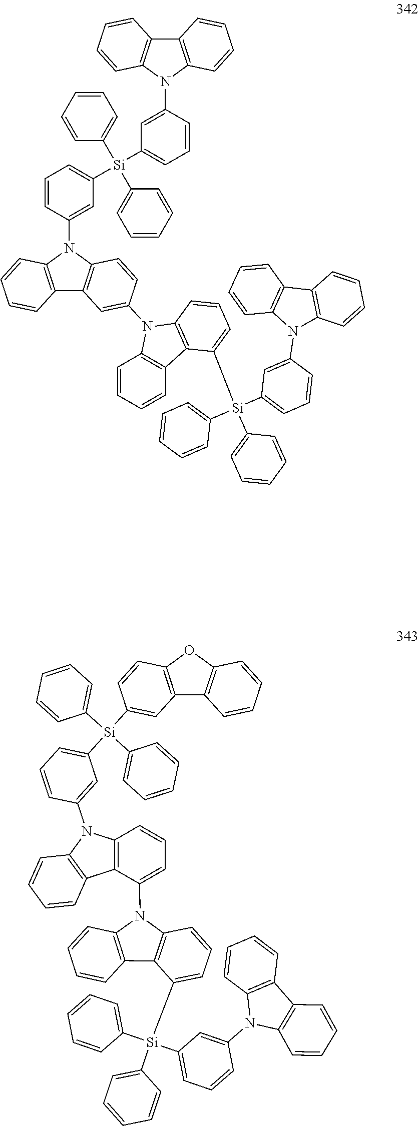 [SMILES: c1ccc([Si](c2ccccc2)(c2cccc(-n3c4ccccc4c4ccccc43)c2)c2cccc(-n3c4ccccc4c4cc(-n5c6ccccc6c6c([Si](c7ccccc7)(c7ccccc7)c7cccc(-n8c9ccccc9c9ccccc98)c7)cccc65)ccc43)c2)cc1 ; c1ccc([Si](c2ccccc2)(c2cccc(-n3c4ccccc4c4c(-n5c6ccccc6c6c([Si](c7ccccc7)(c7ccccc7)c7cccc(-n8c9ccccc9c9ccccc98)c7)cccc65)cccc43)c2)c2ccc3oc4ccccc4c3c2)cc1]

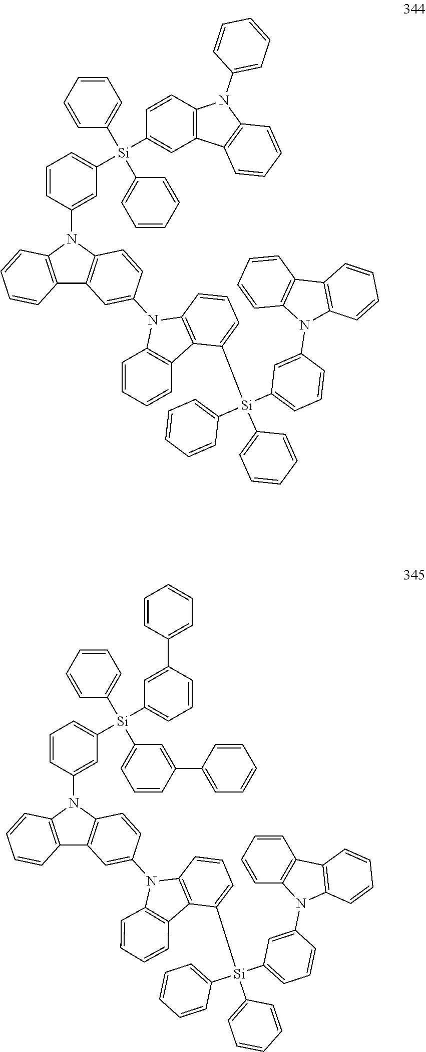 [SMILES: c1ccc(-n2c3ccccc3c3cc([Si](c4ccccc4)(c4ccccc4)c4cccc(-n5c6ccccc6c6cc(-n7c8ccccc8c8c([Si](c9ccccc9)(c9ccccc9)c9cccc(-n%10c%11ccccc%11c%11ccccc%11%10)c9)cccc87)ccc65)c4)ccc32)cc1 ; c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)c2)cc1]

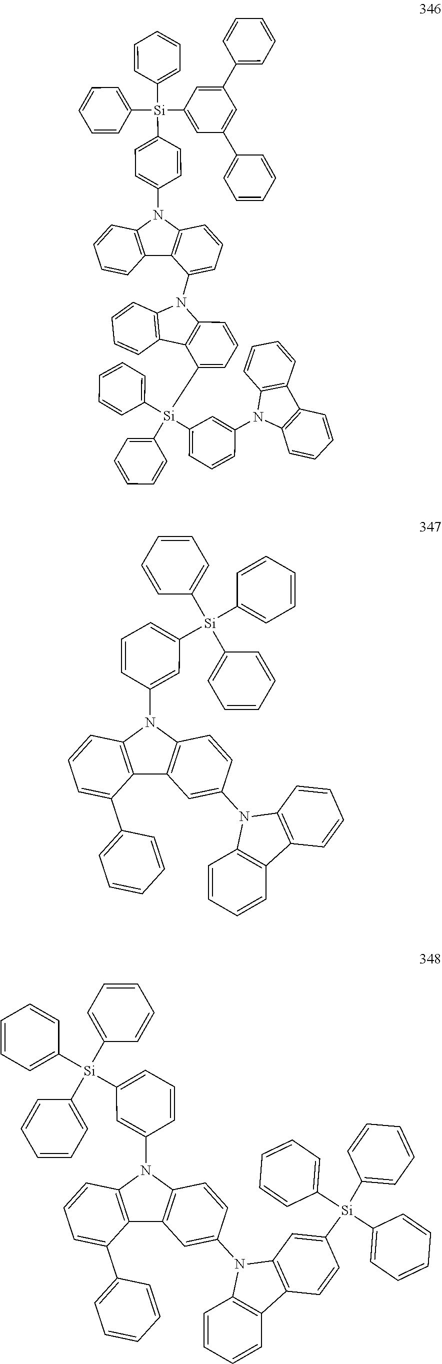 [SMILES: c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)cc3)c2)cc1 ; c1ccc(-c2cccc3c2c2cc(-n4c5ccccc5c5ccccc54)ccc2n3-c2cccc([Si](c3ccccc3)(c3ccccc3)c3ccccc3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccccc3)c2)cc1]

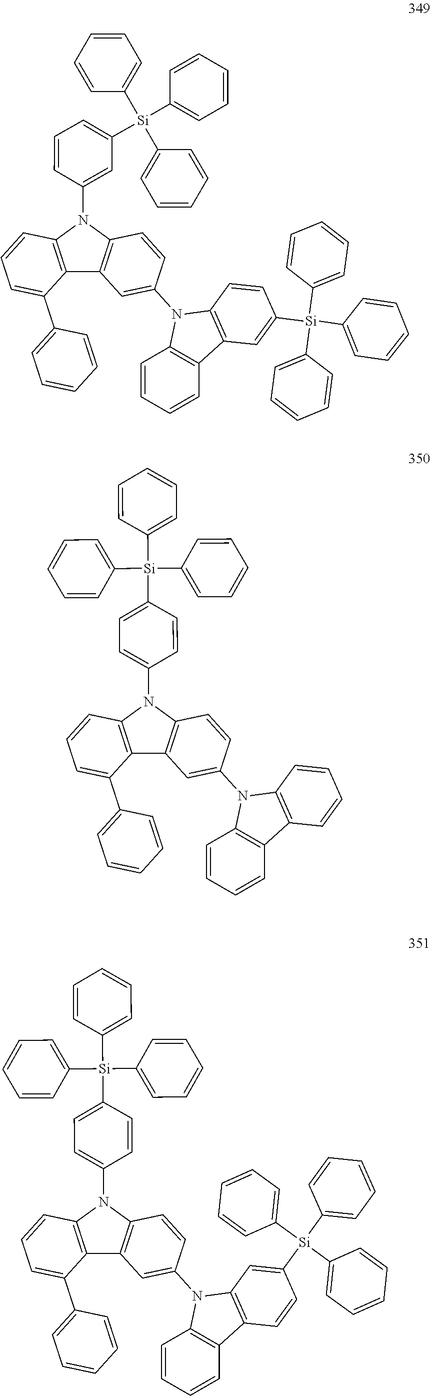 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3ccccc3)c2)cc1 ; c1ccc(-c2cccc3c2c2cc(-n4c5ccccc5c5ccccc54)ccc2n3-c2ccc([Si](c3ccccc3)(c3ccccc3)c3ccccc3)cc2)cc1 ; c1ccc(-c2cccc3c2c2cc(-n4c5ccccc5c5ccc([Si](c6ccccc6)(c6ccccc6)c6ccccc6)cc54)ccc2n3-c2ccc([Si](c3ccccc3)(c3ccccc3)c3ccccc3)cc2)cc1]

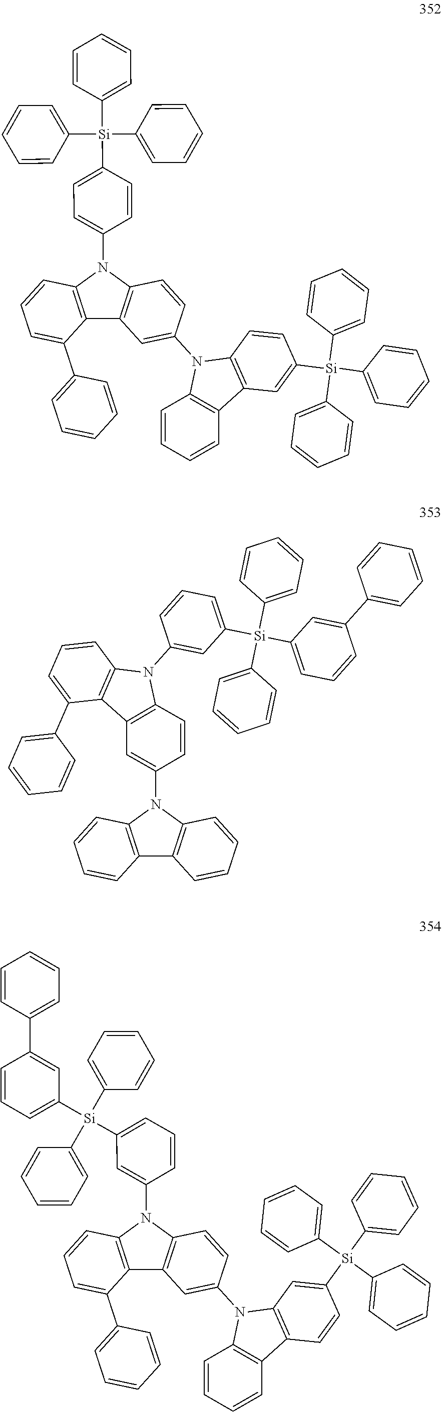 [SMILES: c1ccc(-c2cccc3c2c2cc(-n4c5ccccc5c5cc([Si](c6ccccc6)(c6ccccc6)c6ccccc6)ccc54)ccc2n3-c2ccc([Si](c3ccccc3)(c3ccccc3)c3ccccc3)cc2)cc1 ; c1ccc(-c2cccc([Si](c3ccccc3)(c3ccccc3)c3cccc(-n4c5ccc(-n6c7ccccc7c7ccccc76)cc5c5c(-c6ccccc6)cccc54)c3)c2)cc1 ; c1ccc(-c2cccc([Si](c3ccccc3)(c3ccccc3)c3cccc(-n4c5ccc(-n6c7ccccc7c7ccc([Si](c8ccccc8)(c8ccccc8)c8ccccc8)cc76)cc5c5c(-c6ccccc6)cccc54)c3)c2)cc1]

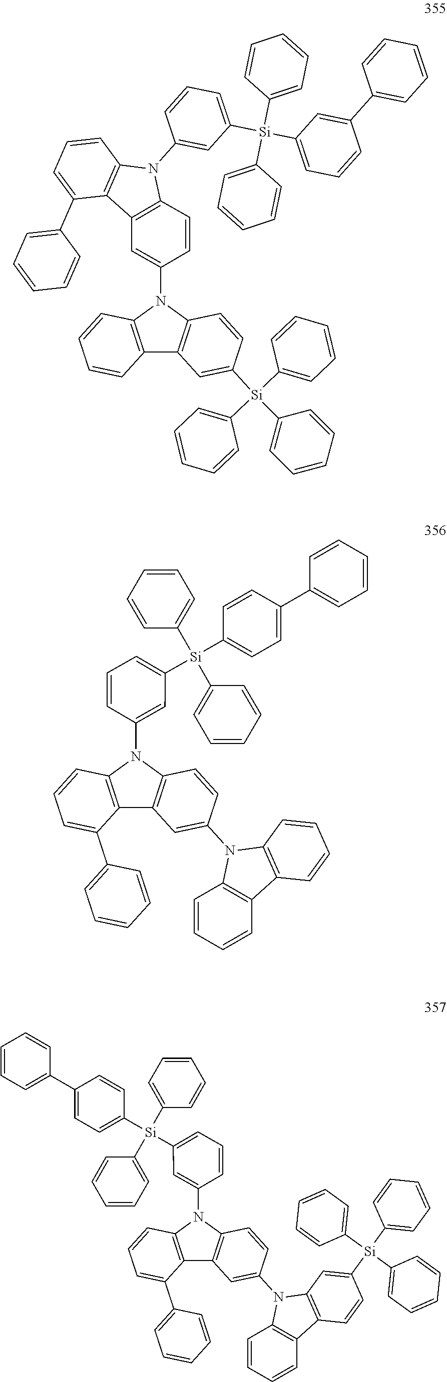 [SMILES: c1ccc(-c2cccc([Si](c3ccccc3)(c3ccccc3)c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1 ; c1ccc(-c2ccc([Si](c3ccccc3)(c3ccccc3)c3cccc(-n4c5ccc(-n6c7ccccc7c7ccccc76)cc5c5c(-c6ccccc6)cccc54)c3)cc2)cc1 ; c1ccc(-c2ccc([Si](c3ccccc3)(c3ccccc3)c3cccc(-n4c5ccc(-n6c7ccccc7c7ccc([Si](c8ccccc8)(c8ccccc8)c8ccccc8)cc76)cc5c5c(-c6ccccc6)cccc54)c3)cc2)cc1]

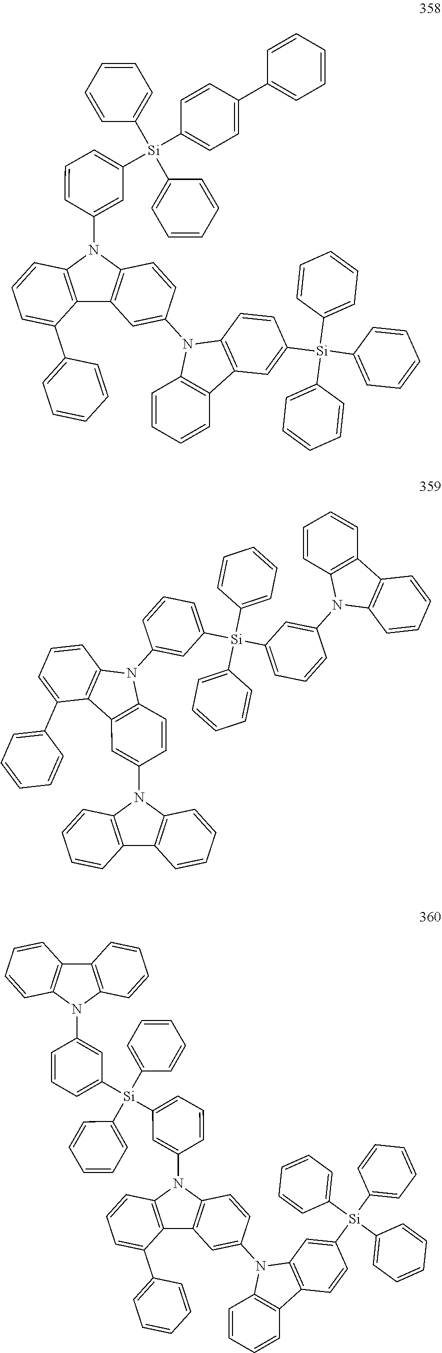 [SMILES: c1ccc(-c2ccc([Si](c3ccccc3)(c3ccccc3)c3cccc(-n4c5ccc(-n6c7ccccc7c7cc([Si](c8ccccc8)(c8ccccc8)c8ccccc8)ccc76)cc5c5c(-c6ccccc6)cccc54)c3)cc2)cc1 ; c1ccc(-c2cccc3c2c2cc(-n4c5ccccc5c5ccccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3cccc(-n4c5ccccc5c5ccccc54)c3)c2)cc1]

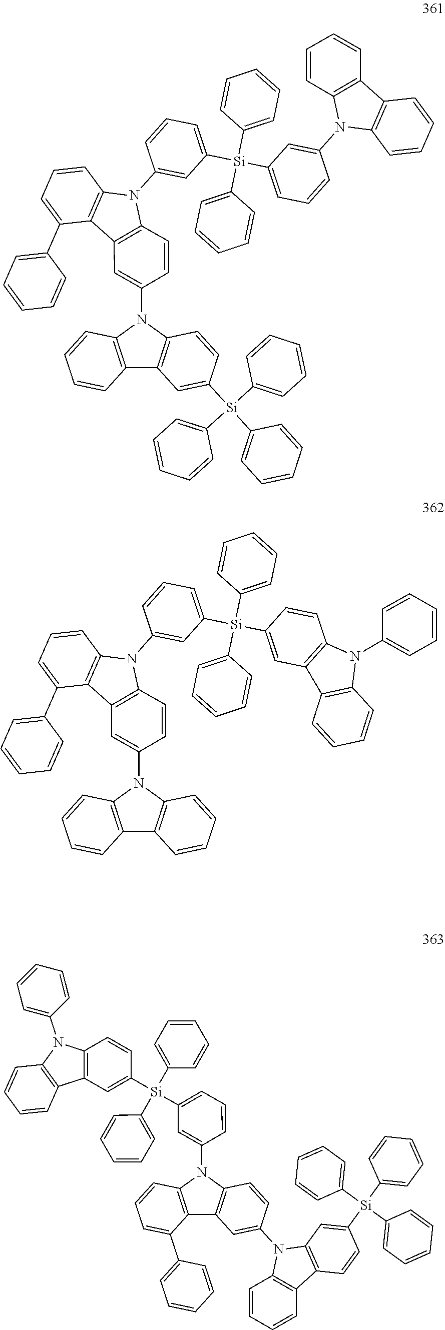 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2cc(-n4c5ccccc5c5ccccc54)ccc2n3-c2cccc([Si](c3ccccc3)(c3ccccc3)c3ccc4c(c3)c3ccccc3n4-c3ccccc3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccc4c(c3)c3ccccc3n4-c3ccccc3)c2)cc1]

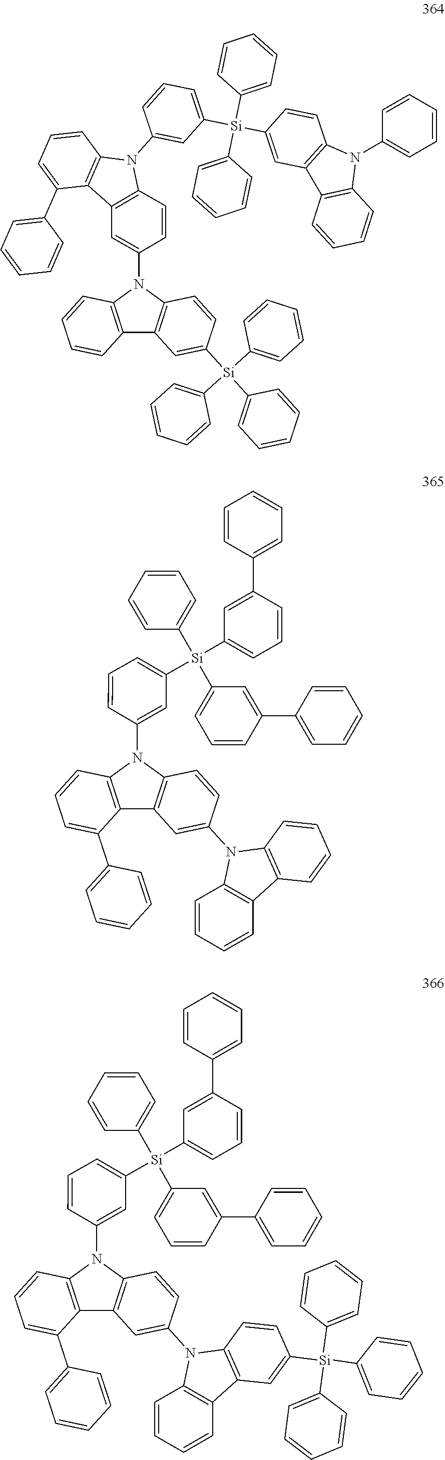

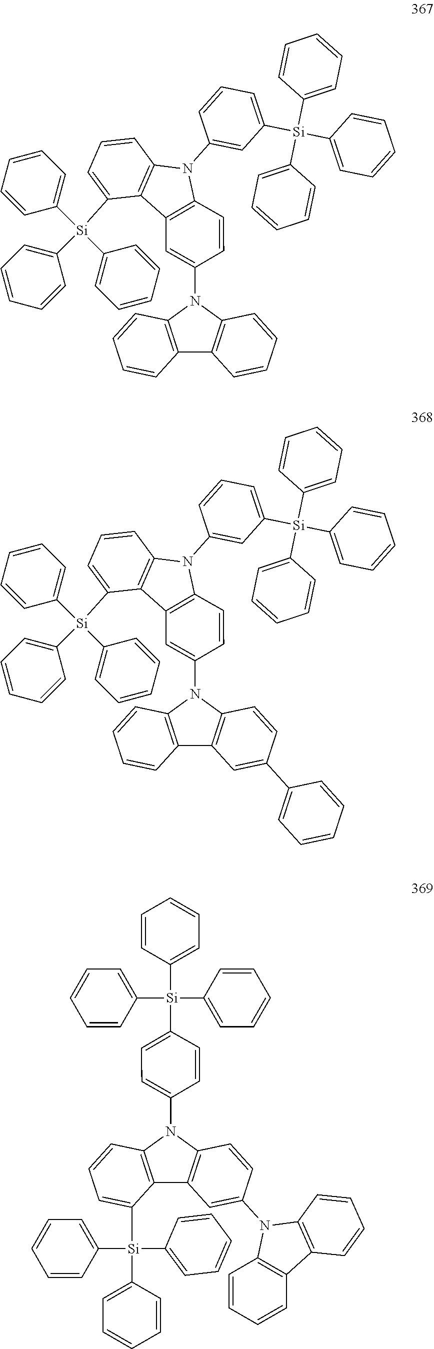 [SMILES: c1ccc([Si](c2ccccc2)(c2ccccc2)c2cccc(-n3c4ccc(-n5c6ccccc6c6ccccc65)cc4c4c([Si](c5ccccc5)(c5ccccc5)c5ccccc5)cccc43)c2)cc1 ; c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)cc1 ; c1ccc([Si](c2ccccc2)(c2ccccc2)c2ccc(-n3c4ccc(-n5c6ccccc6c6ccccc65)cc4c4c([Si](c5ccccc5)(c5ccccc5)c5ccccc5)cccc43)cc2)cc1]

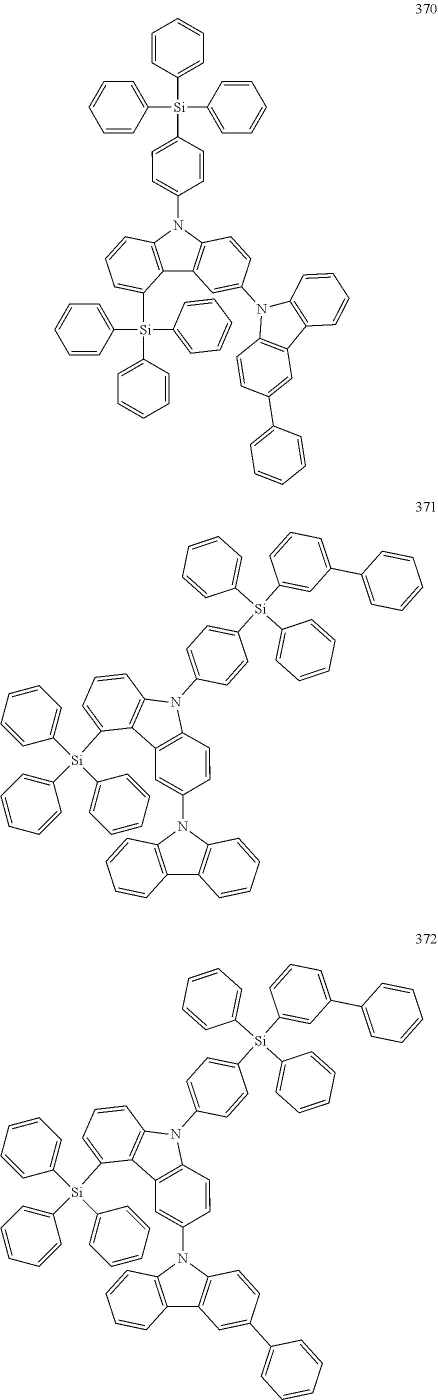 [SMILES: c1ccc(-c2ccc3c(c2)c2ccccc2n3-c2ccc3c(c2)c2c([Si](c4ccccc4)(c4ccccc4)c4ccccc4)cccc2n3-c2ccc([Si](c3ccccc3)(c3ccccc3)c3ccccc3)cc2)cc1 ; c1ccc(-c2cccc([Si](c3ccccc3)(c3ccccc3)c3ccc(-n4c5ccc(-n6c7ccccc7c7ccccc76)cc5c5c([Si](c6ccccc6)(c6ccccc6)c6ccccc6)cccc54)cc3)c2)cc1 ; c1ccc(-c2cccc([Si](c3ccccc3)(c3ccccc3)c3ccc(-n4c5ccc(-n6c7ccccc7c7cc(-c8ccccc8)ccc76)cc5c5c([Si](c6ccccc6)(c6ccccc6)c6ccccc6)cccc54)cc3)c2)cc1]

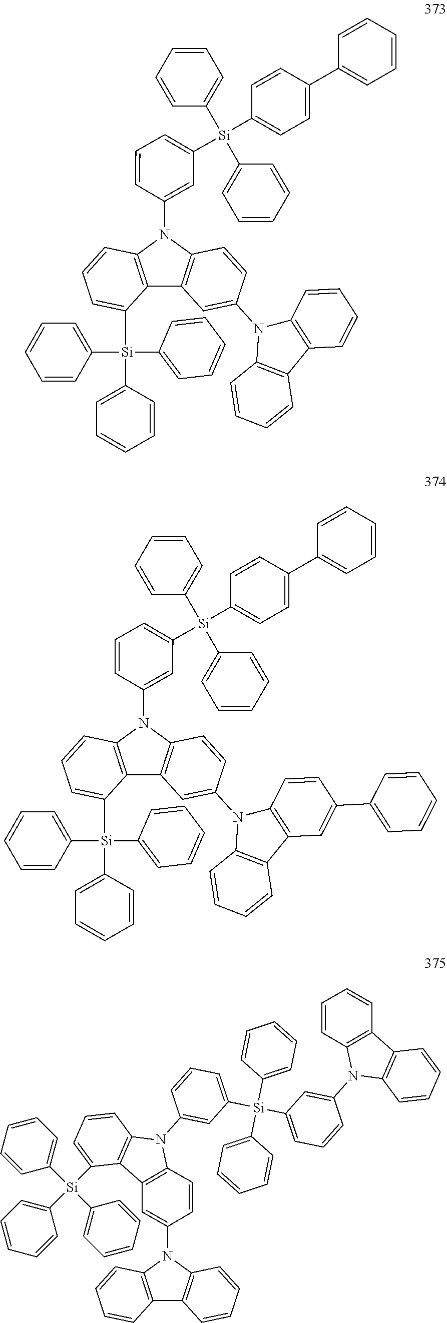 [SMILES: c1ccc(-c2ccc([Si](c3ccccc3)(c3ccccc3)c3cccc(-n4c5ccc(-n6c7ccccc7c7ccccc76)cc5c5c([Si](c6ccccc6)(c6ccccc6)c6ccccc6)cccc54)c3)cc2)cc1 ; c1ccc(-c2ccc([Si](c3ccccc3)(c3ccccc3)c3cccc(-n4c5ccc(-n6c7ccccc7c7cc(-c8ccccc8)ccc76)cc5c5c([Si](c6ccccc6)(c6ccccc6)c6ccccc6)cccc54)c3)cc2)cc1 ; c1ccc([Si](c2ccccc2)(c2cccc(-n3c4ccccc4c4ccccc43)c2)c2cccc(-n3c4ccc(-n5c6ccccc6c6ccccc65)cc4c4c([Si](c5ccccc5)(c5ccccc5)c5ccccc5)cccc43)c2)cc1]

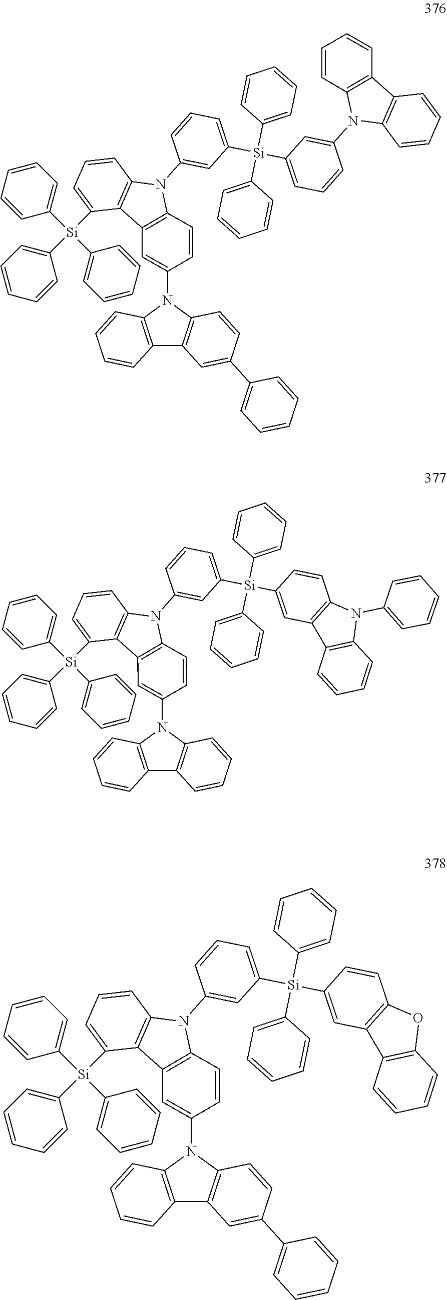 [SMILES: c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3cccc(-n4c5ccccc5c5ccccc54)c3)c2)cc1 ; c1ccc(-n2c3ccccc3c3cc([Si](c4ccccc4)(c4ccccc4)c4cccc(-n5c6ccc(-n7c8ccccc8c8ccccc87)cc6c6c([Si](c7ccccc7)(c7ccccc7)c7ccccc7)cccc65)c4)ccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4oc5ccccc5c4c3)c2)cc1]

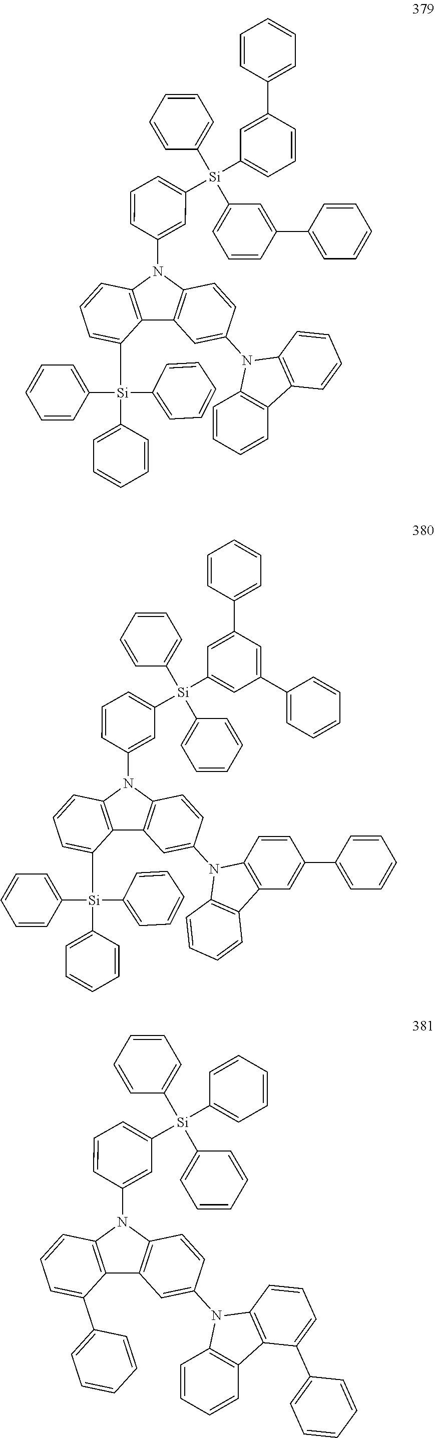 [SMILES: c1ccc(-c2cccc([Si](c3ccccc3)(c3cccc(-c4ccccc4)c3)c3cccc(-n4c5ccc(-n6c7ccccc7c7ccccc76)cc5c5c([Si](c6ccccc6)(c6ccccc6)c6ccccc6)cccc54)c3)c2)cc1 ; c1ccc(-c2cc(-c3ccccc3)cc([Si](c3ccccc3)(c3ccccc3)c3cccc(-n4c5ccc(-n6c7ccccc7c7cc(-c8ccccc8)ccc76)cc5c5c([Si](c6ccccc6)(c6ccccc6)c6ccccc6)cccc54)c3)c2)cc1 ; c1ccc(-c2cccc3c2c2ccccc2n3-c2ccc3c(c2)c2c(-c4ccccc4)cccc2n3-c2cccc([Si](c3ccccc3)(c3ccccc3)c3ccccc3)c2)cc1]

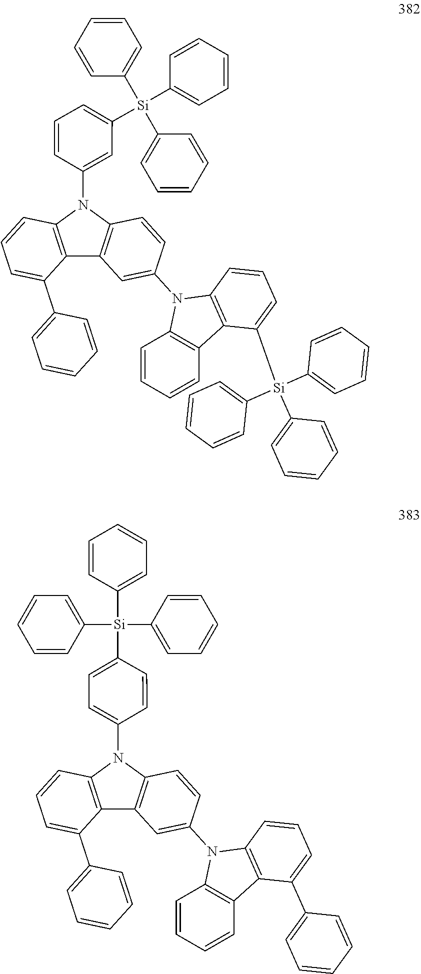 [SMILES: c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3ccccc3)c2)cc1 ; c1ccc(-c2cccc3c2c2ccccc2n3-c2ccc3c(c2)c2c(-c4ccccc4)cccc2n3-c2ccc([Si](c3ccccc3)(c3ccccc3)c3ccccc3)cc2)cc1]

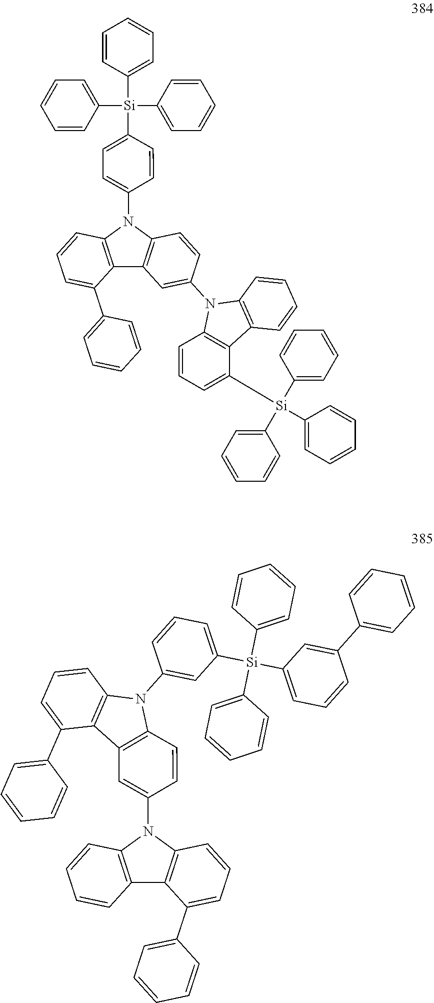 [SMILES: c1ccc(-c2cccc3c2c2cc(-n4c5ccccc5c5c([Si](c6ccccc6)(c6ccccc6)c6ccccc6)cccc54)ccc2n3-c2ccc([Si](c3ccccc3)(c3ccccc3)c3ccccc3)cc2)cc1 ; c1ccc(-c2cccc([Si](c3ccccc3)(c3ccccc3)c3cccc(-n4c5ccc(-n6c7ccccc7c7c(-c8ccccc8)cccc76)cc5c5c(-c6ccccc6)cccc54)c3)c2)cc1]

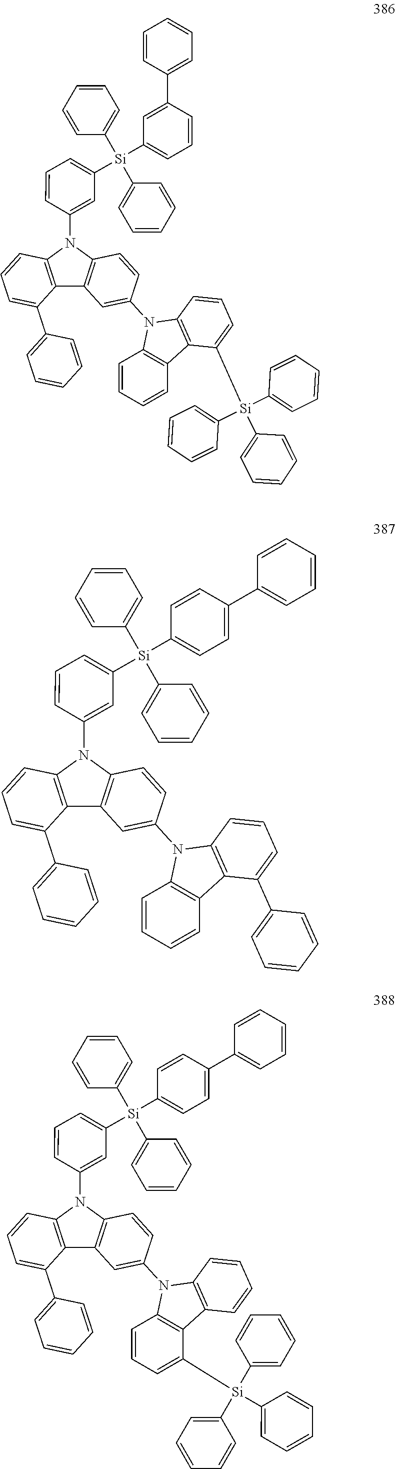 [SMILES: c1ccc(-c2cccc([Si](c3ccccc3)(c3ccccc3)c3cccc(-n4c5ccc(-n6c7ccccc7c7c([Si](c8ccccc8)(c8ccccc8)c8ccccc8)cccc76)cc5c5c(-c6ccccc6)cccc54)c3)c2)cc1 ; c1ccc(-c2ccc([Si](c3ccccc3)(c3ccccc3)c3cccc(-n4c5ccc(-n6c7ccccc7c7c(-c8ccccc8)cccc76)cc5c5c(-c6ccccc6)cccc54)c3)cc2)cc1 ; c1ccc(-c2ccc([Si](c3ccccc3)(c3ccccc3)c3cccc(-n4c5ccc(-n6c7ccccc7c7c([Si](c8ccccc8)(c8ccccc8)c8ccccc8)cccc76)cc5c5c(-c6ccccc6)cccc54)c3)cc2)cc1]

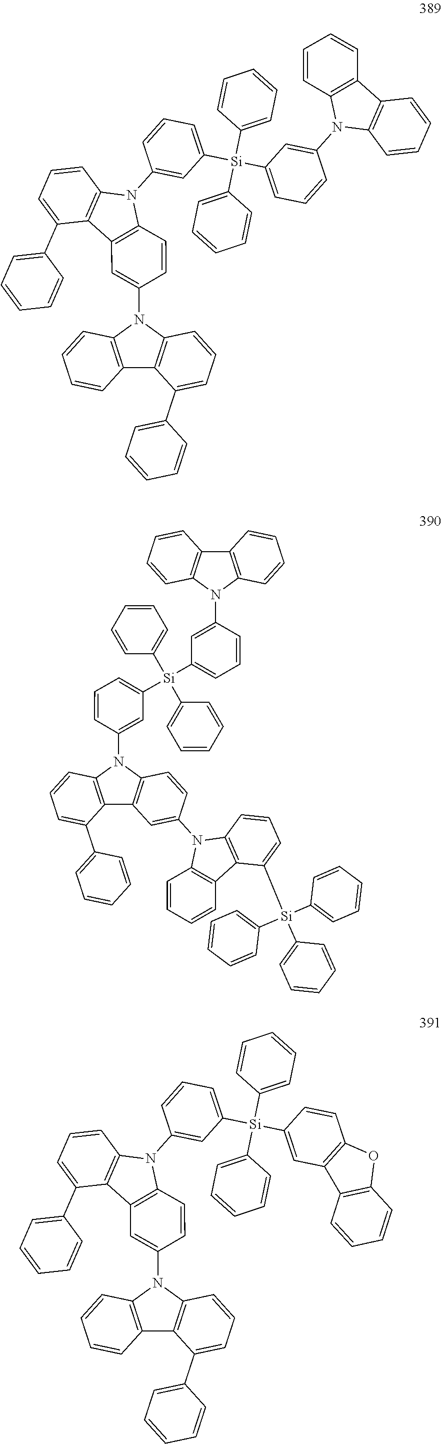 [SMILES: c1ccc(-c2cccc3c2c2ccccc2n3-c2ccc3c(c2)c2c(-c4ccccc4)cccc2n3-c2cccc([Si](c3ccccc3)(c3ccccc3)c3cccc(-n4c5ccccc5c5ccccc54)c3)c2)cc1 ; c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2ccccc2n3-c2ccc3c(c2)c2c(-c4ccccc4)cccc2n3-c2cccc([Si](c3ccccc3)(c3ccccc3)c3ccc4oc5ccccc5c4c3)c2)cc1]

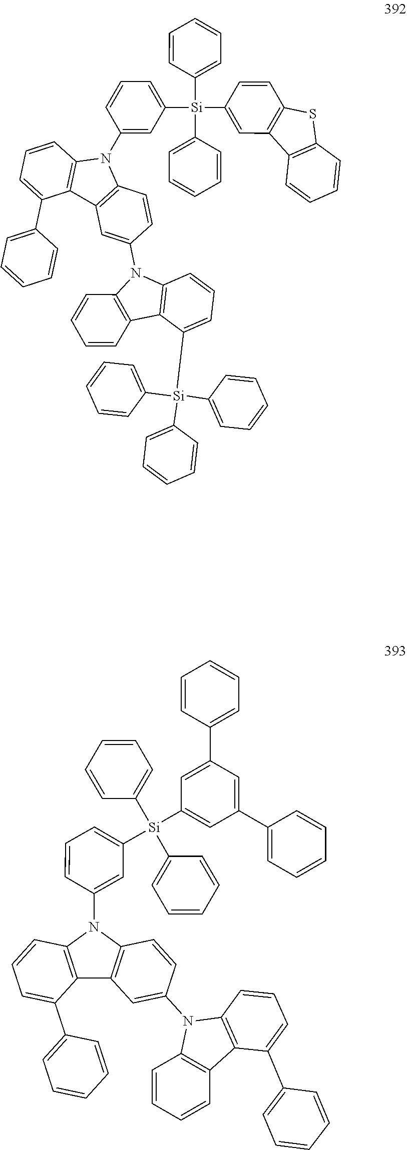 [SMILES: c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3ccc4sc5ccccc5c4c3)c2)cc1 ; c1ccc(-c2cc(-c3ccccc3)cc([Si](c3ccccc3)(c3ccccc3)c3cccc(-n4c5ccc(-n6c7ccccc7c7c(-c8ccccc8)cccc76)cc5c5c(-c6ccccc6)cccc54)c3)c2)cc1]

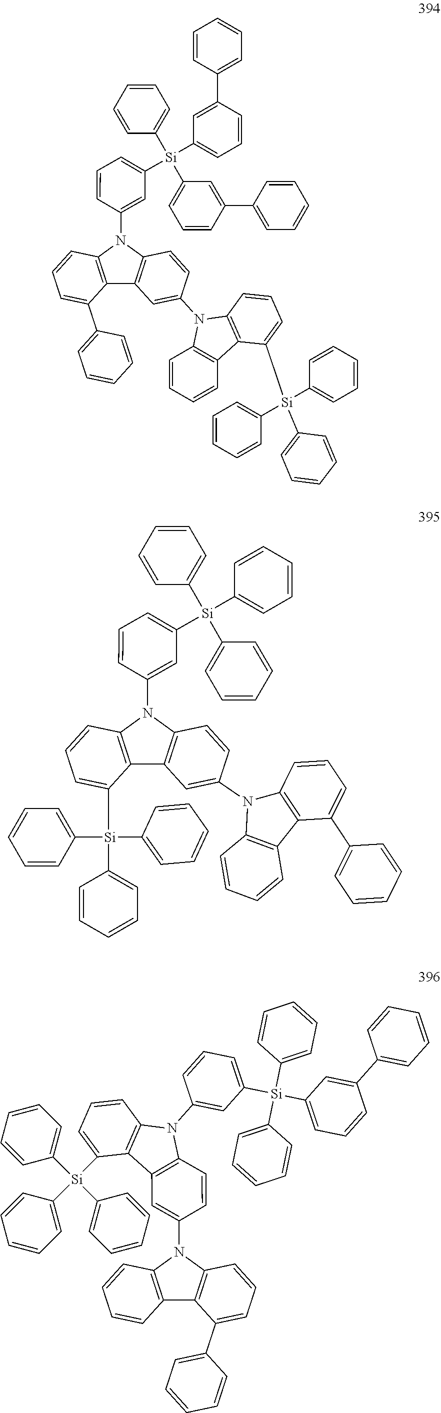 [SMILES: c1ccc(-c2cccc([Si](c3ccccc3)(c3cccc(-c4ccccc4)c3)c3cccc(-n4c5ccc(-n6c7ccccc7c7c([Si](c8ccccc8)(c8ccccc8)c8ccccc8)cccc76)cc5c5c(-c6ccccc6)cccc54)c3)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)cc1 ; c1ccc(-c2cccc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1]

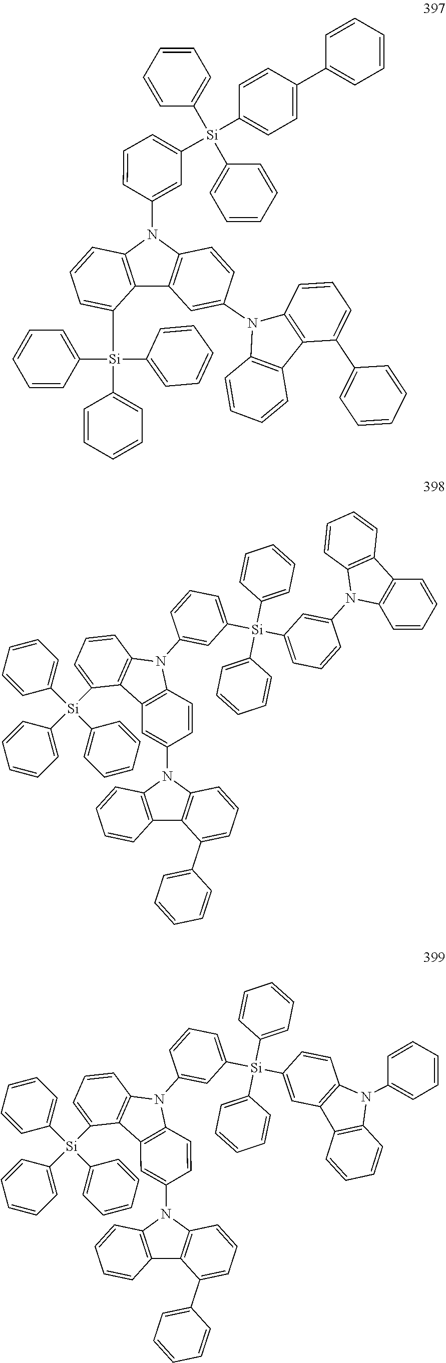 [SMILES: c1ccc(-c2ccc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)cc2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3cccc(-n4c5ccccc5c5ccccc54)c3)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4c(c3)c3ccccc3n4-c3ccccc3)c2)cc1]

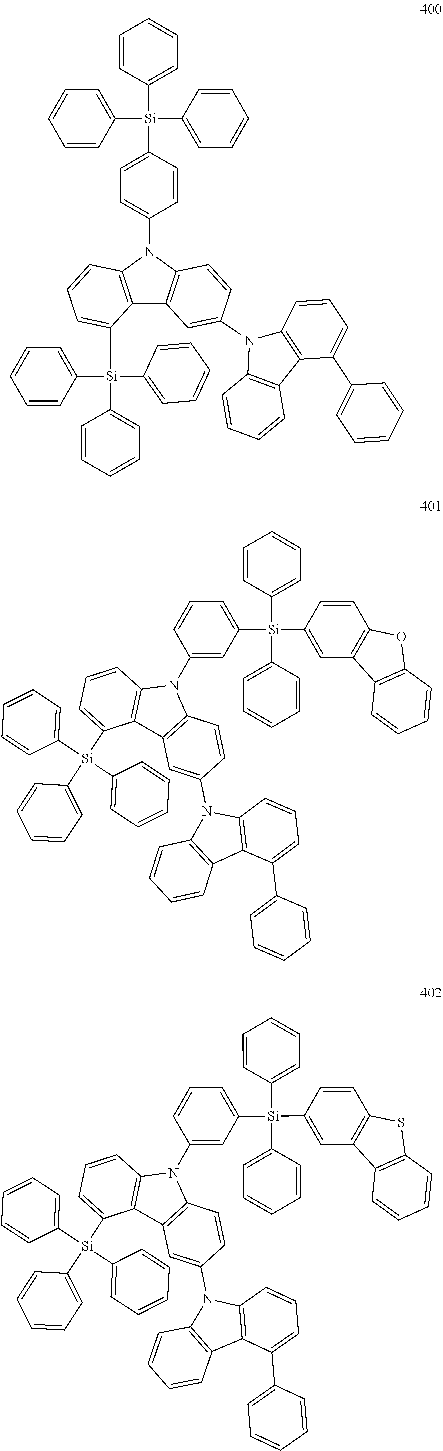 [SMILES: c1ccc(-c2cccc3c2c2ccccc2n3-c2ccc3c(c2)c2c([Si](c4ccccc4)(c4ccccc4)c4ccccc4)cccc2n3-c2ccc([Si](c3ccccc3)(c3ccccc3)c3ccccc3)cc2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4oc5ccccc5c4c3)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4sc5ccccc5c4c3)c2)cc1]

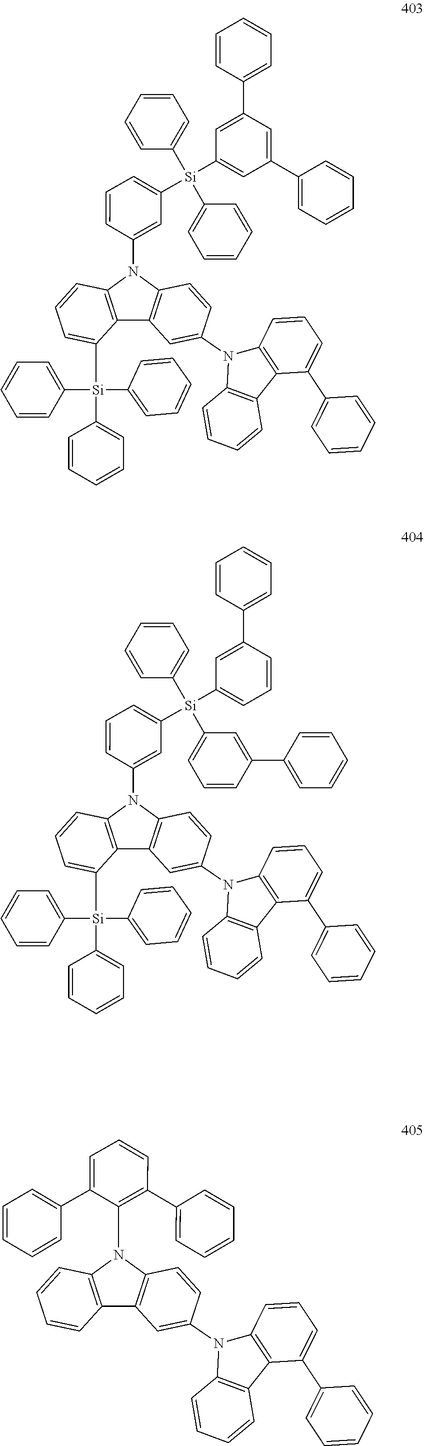 [SMILES: c1ccc(-c2cc(-c3ccccc3)cc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1 ; c1ccc(-c2cccc([Si](c3ccccc3)(c3cccc(-c4ccccc4)c3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1 ; c1ccc(-c2cccc(-c3ccccc3)c2-n2c3ccccc3c3cc(-n4c5ccccc5c5c(-c6ccccc6)cccc54)ccc32)cc1]

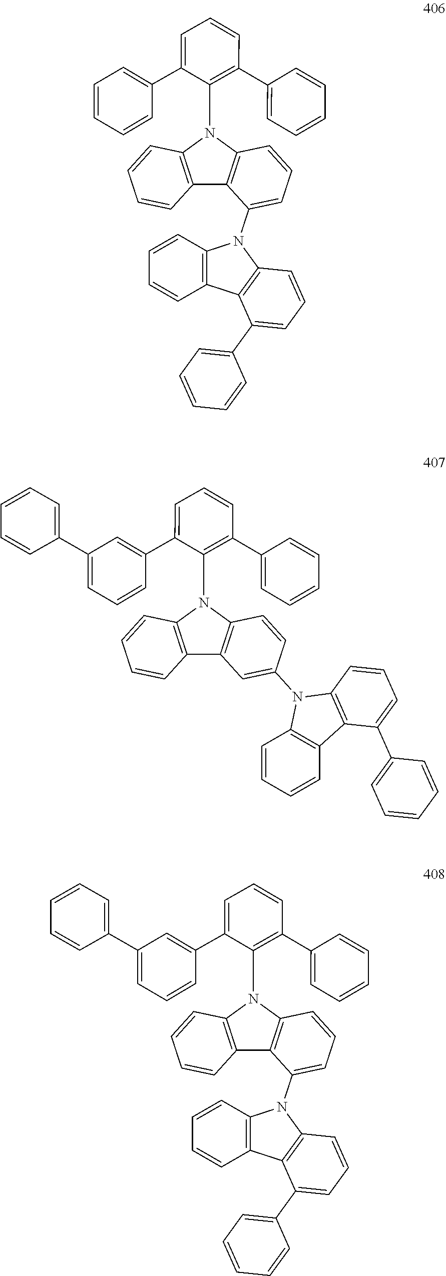 [SMILES: c1ccc(-c2cccc(-c3ccccc3)c2-n2c3ccccc3c3c(-n4c5ccccc5c5c(-c6ccccc6)cccc54)cccc32)cc1 ; c1ccc(-c2cccc(-c3cccc(-c4ccccc4)c3-n3c4ccccc4c4cc(-n5c6ccccc6c6c(-c7ccccc7)cccc65)ccc43)c2)cc1 ; c1ccc(-c2cccc(-c3cccc(-c4ccccc4)c3-n3c4ccccc4c4c(-n5c6ccccc6c6c(-c7ccccc7)cccc65)cccc43)c2)cc1]

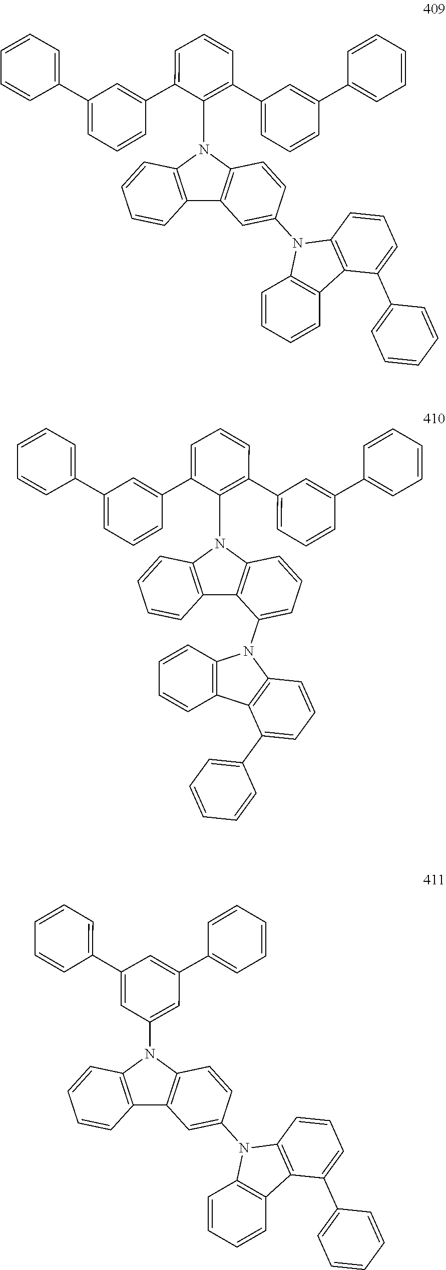 [SMILES: c1ccc(-c2cccc(-c3cccc(-c4cccc(-c5ccccc5)c4)c3-n3c4ccccc4c4cc(-n5c6ccccc6c6c(-c7ccccc7)cccc65)ccc43)c2)cc1 ; c1ccc(-c2cccc(-c3cccc(-c4cccc(-c5ccccc5)c4)c3-n3c4ccccc4c4c(-n5c6ccccc6c6c(-c7ccccc7)cccc65)cccc43)c2)cc1 ; c1ccc(-c2cc(-c3ccccc3)cc(-n3c4ccccc4c4cc(-n5c6ccccc6c6c(-c7ccccc7)cccc65)ccc43)c2)cc1]

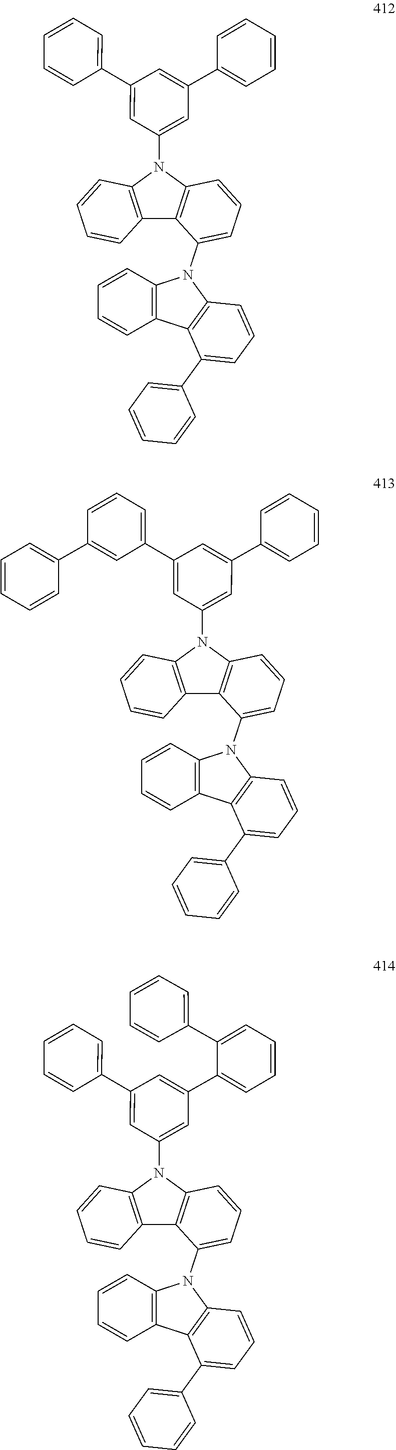 [SMILES: c1ccc(-c2cc(-c3ccccc3)cc(-n3c4ccccc4c4c(-n5c6ccccc6c6c(-c7ccccc7)cccc65)cccc43)c2)cc1 ; c1ccc(-c2cccc(-c3cc(-c4ccccc4)cc(-n4c5ccccc5c5c(-n6c7ccccc7c7c(-c8ccccc8)cccc76)cccc54)c3)c2)cc1 ; c1ccc(-c2cc(-c3ccccc3-c3ccccc3)cc(-n3c4ccccc4c4c(-n5c6ccccc6c6c(-c7ccccc7)cccc65)cccc43)c2)cc1]

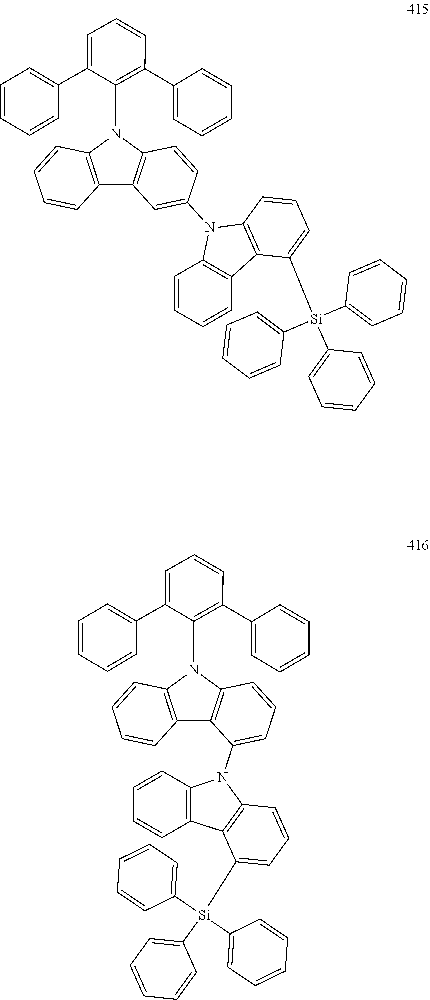 [SMILES: c1ccc(-c2cccc(-c3ccccc3)c2-n2c3ccccc3c3cc(-n4c5ccccc5c5c([Si](c6ccccc6)(c6ccccc6)c6ccccc6)cccc54)ccc32)cc1 ; c1ccc(-c2cccc(-c3ccccc3)c2-n2c3ccccc3c3c(-n4c5ccccc5c5c([Si](c6ccccc6)(c6ccccc6)c6ccccc6)cccc54)cccc32)cc1]

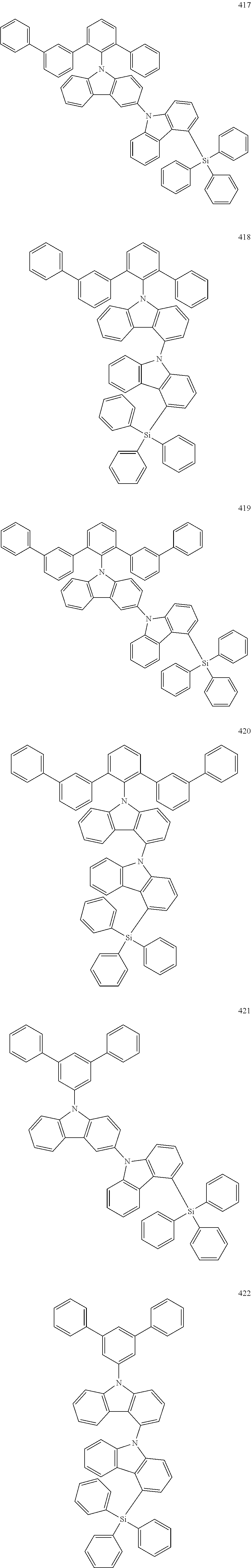

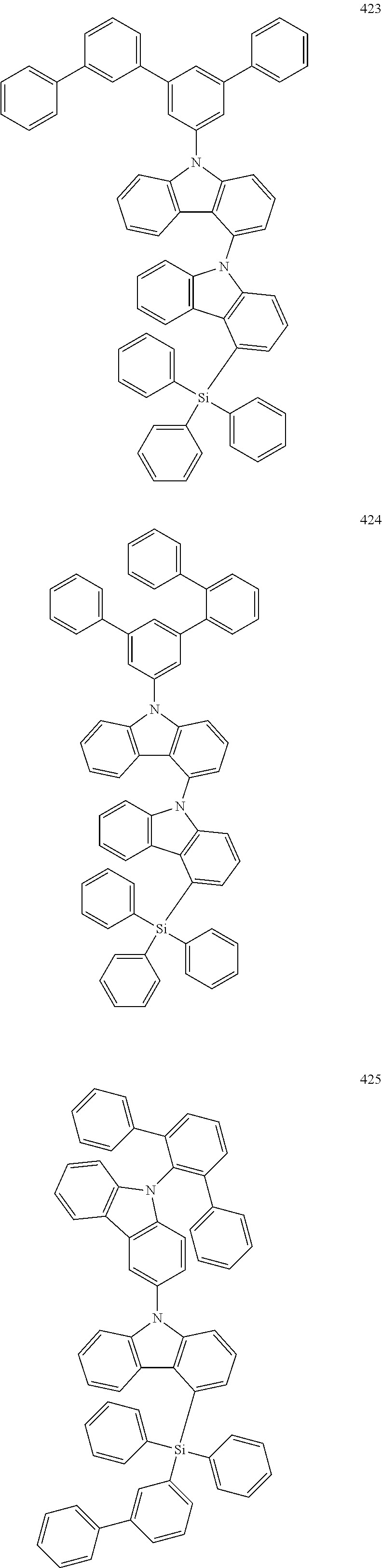 [SMILES: c1ccc(-c2cccc(-c3cc(-c4ccccc4)cc(-n4c5ccccc5c5c(-n6c7ccccc7c7c([Si](c8ccccc8)(c8ccccc8)c8ccccc8)cccc76)cccc54)c3)c2)cc1 ; c1ccc(-c2cc(-c3ccccc3-c3ccccc3)cc(-n3c4ccccc4c4c(-n5c6ccccc6c6c([Si](c7ccccc7)(c7ccccc7)c7ccccc7)cccc65)cccc43)c2)cc1 ; c1ccc(-c2cccc([Si](c3ccccc3)(c3ccccc3)c3cccc4c3c3ccccc3n4-c3ccc4c(c3)c3ccccc3n4-c3c(-c4ccccc4)cccc3-c3ccccc3)c2)cc1]

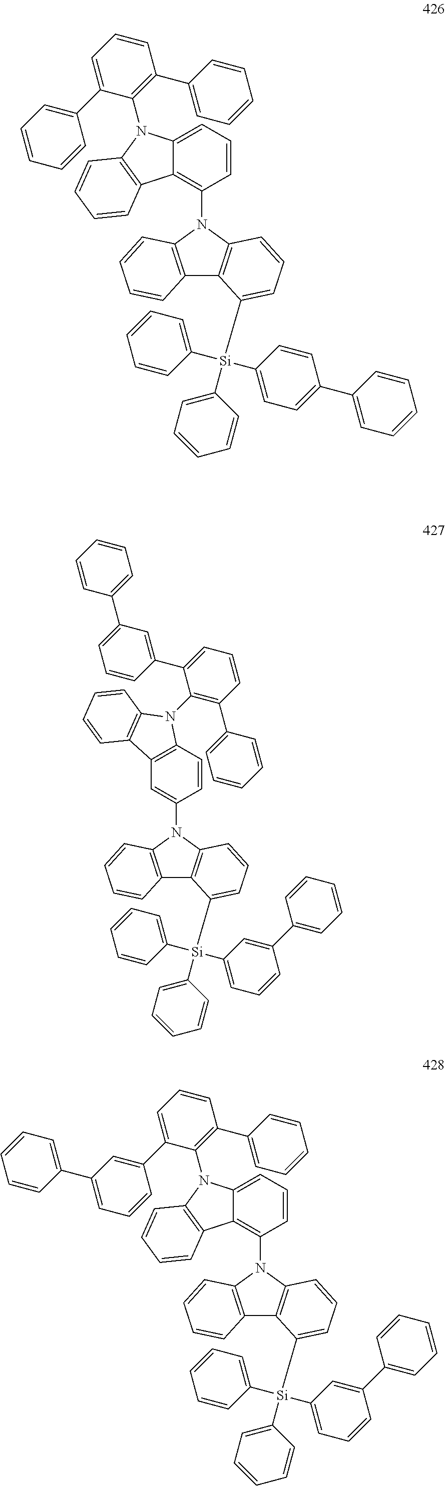 [SMILES: c1ccc(-c2ccc([Si](c3ccccc3)(c3ccccc3)c3cccc4c3c3ccccc3n4-c3cccc4c3c3ccccc3n4-c3c(-c4ccccc4)cccc3-c3ccccc3)cc2)cc1 ; c1ccc(-c2cccc(-c3cccc(-c4ccccc4)c3-n3c4ccccc4c4cc(-n5c6ccccc6c6c([Si](c7ccccc7)(c7ccccc7)c7cccc(-c8ccccc8)c7)cccc65)ccc43)c2)cc1 ; c1ccc(-c2cccc(-c3cccc(-c4ccccc4)c3-n3c4ccccc4c4c(-n5c6ccccc6c6c([Si](c7ccccc7)(c7ccccc7)c7cccc(-c8ccccc8)c7)cccc65)cccc43)c2)cc1]

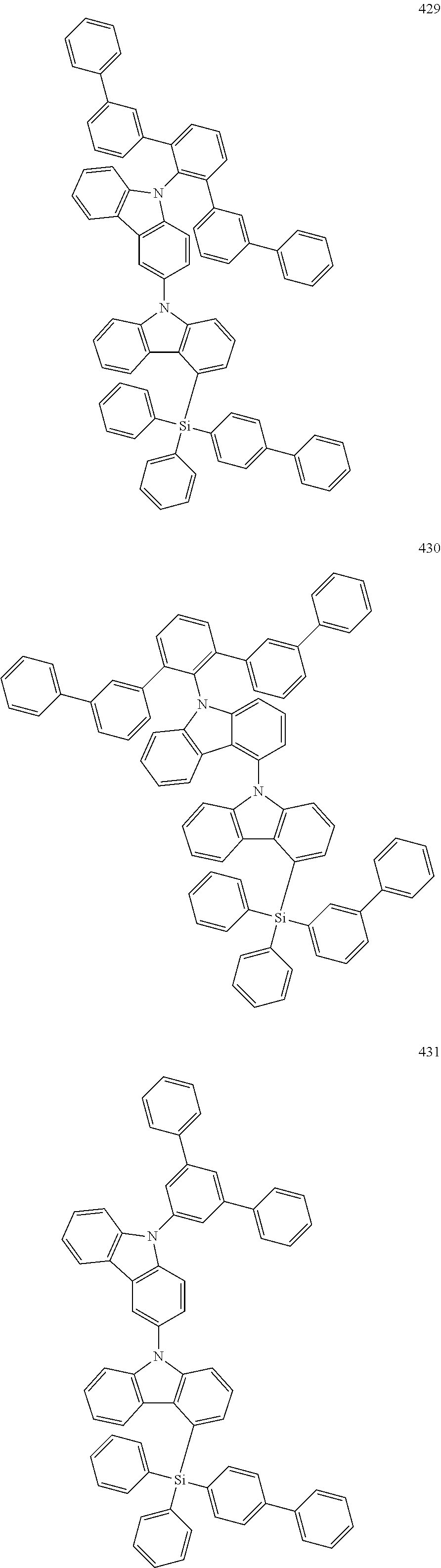 [SMILES: c1ccc(-c2ccc([Si](c3ccccc3)(c3ccccc3)c3cccc4c3c3ccccc3n4-c3ccc4c(c3)c3ccccc3n4-c3c(-c4cccc(-c5ccccc5)c4)cccc3-c3cccc(-c4ccccc4)c3)cc2)cc1 ; c1ccc(-c2cccc(-c3cccc(-c4cccc(-c5ccccc5)c4)c3-n3c4ccccc4c4c(-n5c6ccccc6c6c([Si](c7ccccc7)(c7ccccc7)c7cccc(-c8ccccc8)c7)cccc65)cccc43)c2)cc1 ; c1ccc(-c2ccc([Si](c3ccccc3)(c3ccccc3)c3cccc4c3c3ccccc3n4-c3ccc4c(c3)c3ccccc3n4-c3cc(-c4ccccc4)cc(-c4ccccc4)c3)cc2)cc1]

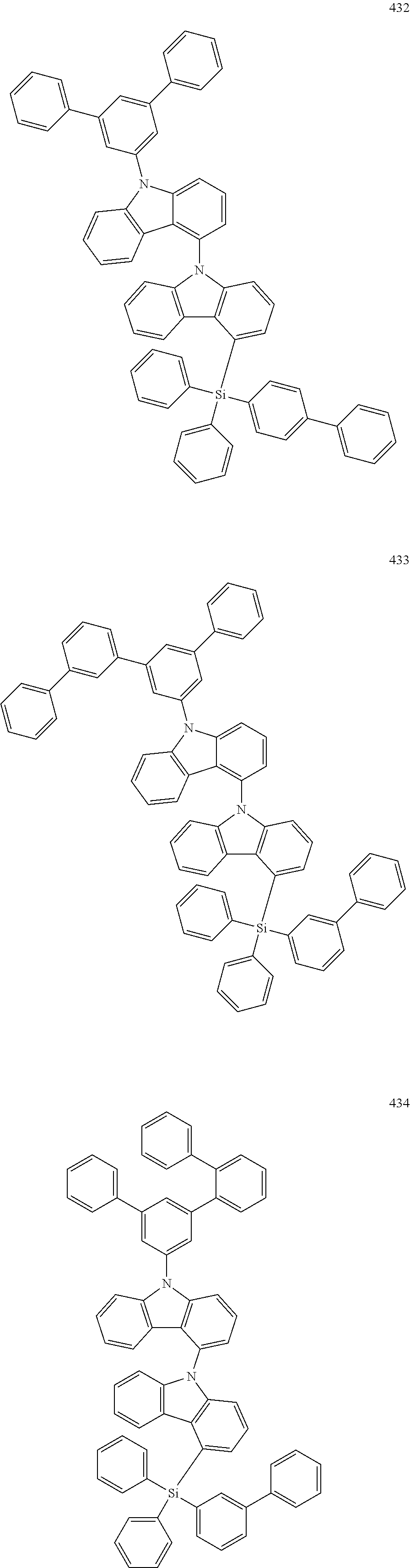 [SMILES: c1ccc(-c2ccc([Si](c3ccccc3)(c3ccccc3)c3cccc4c3c3ccccc3n4-c3cccc4c3c3ccccc3n4-c3cc(-c4ccccc4)cc(-c4ccccc4)c3)cc2)cc1 ; c1ccc(-c2cccc(-c3cc(-c4ccccc4)cc(-n4c5ccccc5c5c(-n6c7ccccc7c7c([Si](c8ccccc8)(c8ccccc8)c8cccc(-c9ccccc9)c8)cccc76)cccc54)c3)c2)cc1 ; c1ccc(-c2cc(-c3ccccc3-c3ccccc3)cc(-n3c4ccccc4c4c(-n5c6ccccc6c6c([Si](c7ccccc7)(c7ccccc7)c7cccc(-c8ccccc8)c7)cccc65)cccc43)c2)cc1]

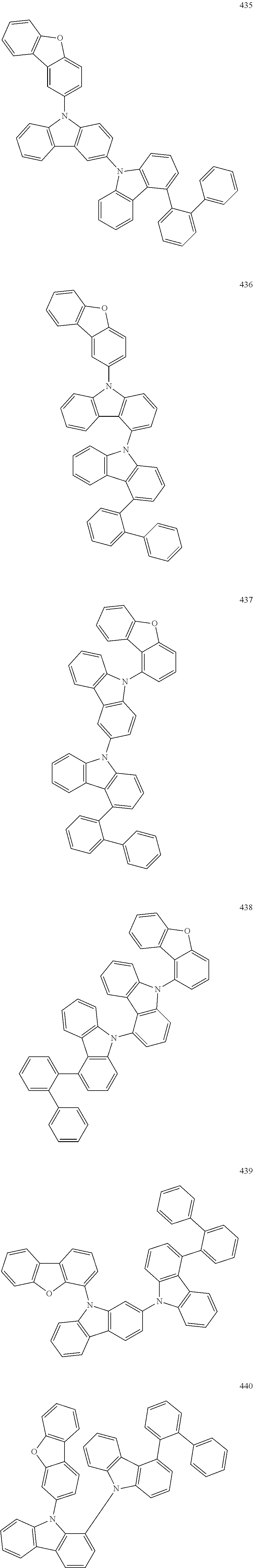 [SMILES: c1ccc(-c2ccccc2-c2cccc3c2c2ccccc2n3-c2ccc3c(c2)c2ccccc2n3-c2ccc3oc4ccccc4c3c2)cc1 ; c1ccc(-c2ccccc2-c2cccc3c2c2ccccc2n3-c2cccc3c2c2ccccc2n3-c2ccc3oc4ccccc4c3c2)cc1 ; c1ccc(-c2ccccc2-c2cccc3c2c2ccccc2n3-c2ccc3c(c2)c2ccccc2n3-c2cccc3oc4ccccc4c23)cc1 ; c1ccc(-c2ccccc2-c2cccc3c2c2ccccc2n3-c2cccc3c2c2ccccc2n3-c2cccc3oc4ccccc4c23)cc1 ; c1ccc(-c2ccccc2-c2cccc3c2c2ccccc2n3-c2ccc3c4ccccc4n(-c4cccc5c4oc4ccccc45)c3c2)cc1 ; c1ccc(-c2ccccc2-c2cccc3c2c2ccccc2n3-c2cccc3c4ccccc4n(-c4ccc5c(c4)oc4ccccc45)c23)cc1]

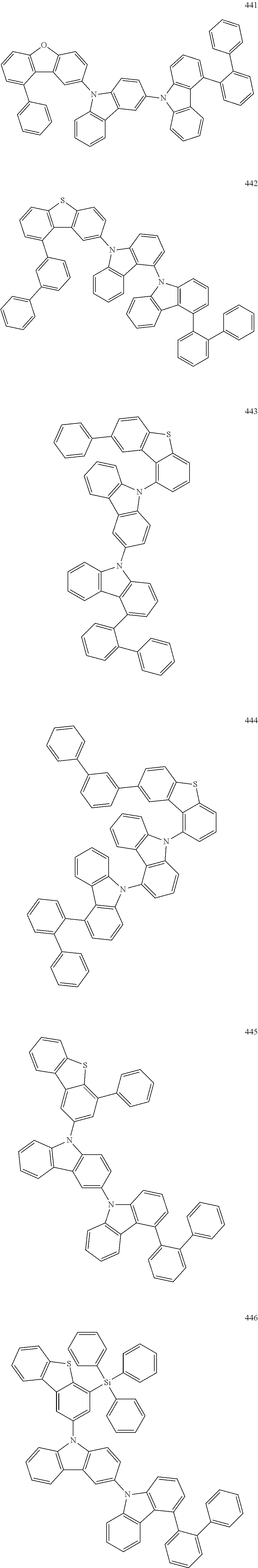

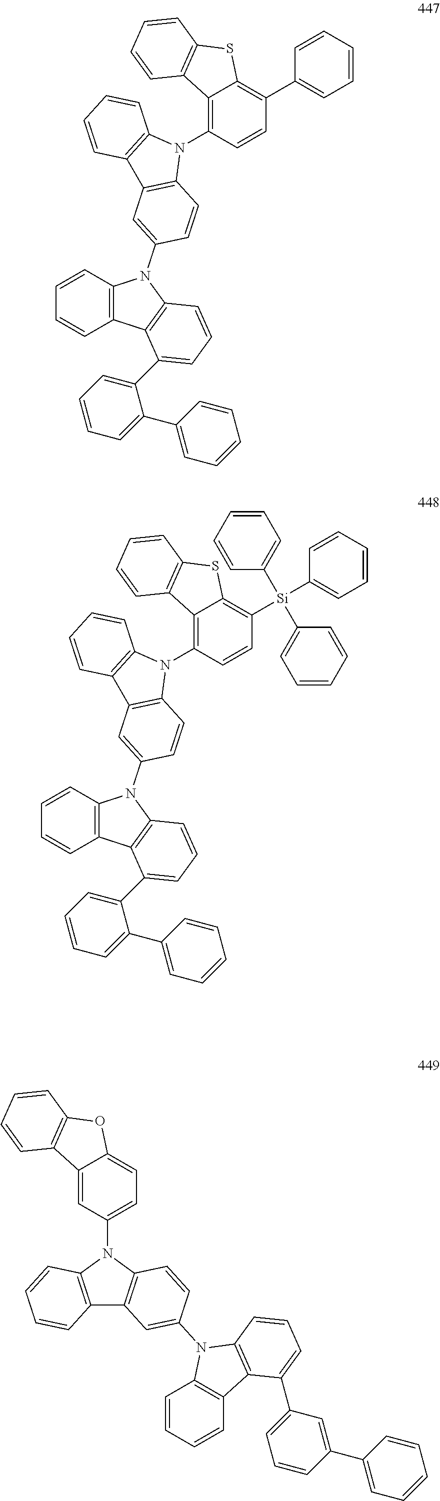 [SMILES: c1ccc(-c2ccccc2-c2cccc3c2c2ccccc2n3-c2ccc3c(c2)c2ccccc2n3-c2ccc(-c3ccccc3)c3sc4ccccc4c23)cc1 ; c1ccc(-c2ccccc2-c2cccc3c2c2ccccc2n3-c2ccc3c(c2)c2ccccc2n3-c2ccc([Si](c3ccccc3)(c3ccccc3)c3ccccc3)c3sc4ccccc4c23)cc1 ; c1ccc(-c2cccc(-c3cccc4c3c3ccccc3n4-c3ccc4c(c3)c3ccccc3n4-c3ccc4oc5ccccc5c4c3)c2)cc1]

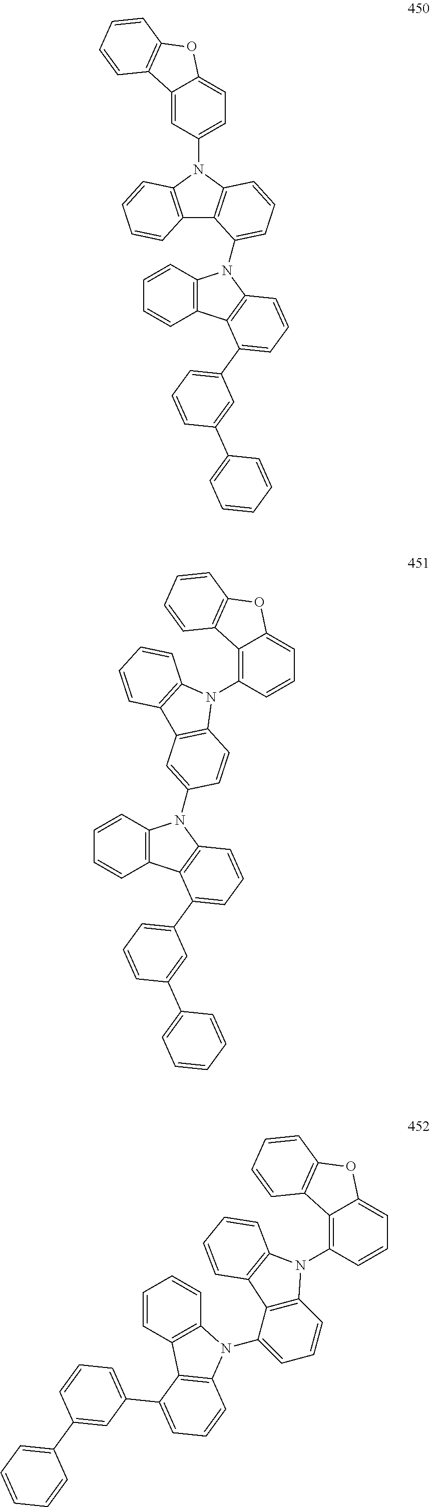 [SMILES: c1ccc(-c2cccc(-c3cccc4c3c3ccccc3n4-c3cccc4c3c3ccccc3n4-c3ccc4oc5ccccc5c4c3)c2)cc1 ; c1ccc(-c2cccc(-c3cccc4c3c3ccccc3n4-c3ccc4c(c3)c3ccccc3n4-c3cccc4oc5ccccc5c34)c2)cc1 ; c1ccc(-c2cccc(-c3cccc4c3c3ccccc3n4-c3cccc4c3c3ccccc3n4-c3cccc4oc5ccccc5c34)c2)cc1]

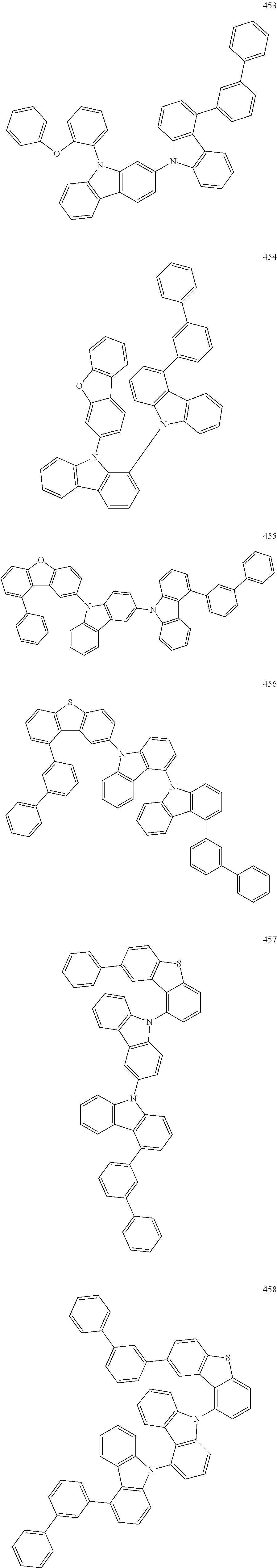

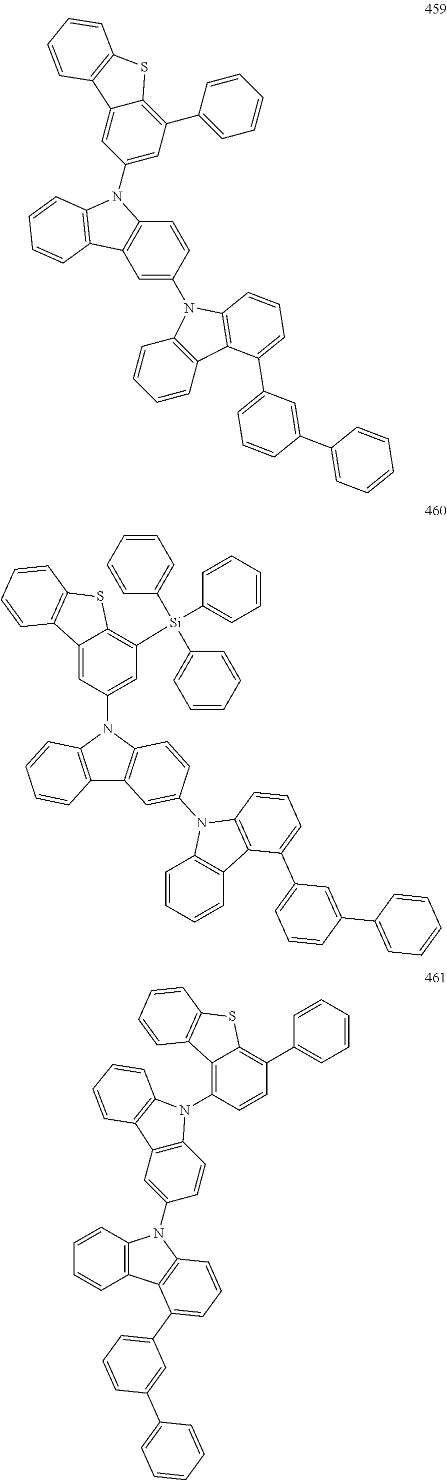 [SMILES: c1ccc(-c2cccc(-c3cccc4c3c3ccccc3n4-c3ccc4c(c3)c3ccccc3n4-c3cc(-c4ccccc4)c4sc5ccccc5c4c3)c2)cc1 ; c1ccc(-c2cccc(-c3cccc4c3c3ccccc3n4-c3ccc4c(c3)c3ccccc3n4-c3cc([Si](c4ccccc4)(c4ccccc4)c4ccccc4)c4sc5ccccc5c4c3)c2)cc1 ; c1ccc(-c2cccc(-c3cccc4c3c3ccccc3n4-c3ccc4c(c3)c3ccccc3n4-c3ccc(-c4ccccc4)c4sc5ccccc5c34)c2)cc1]

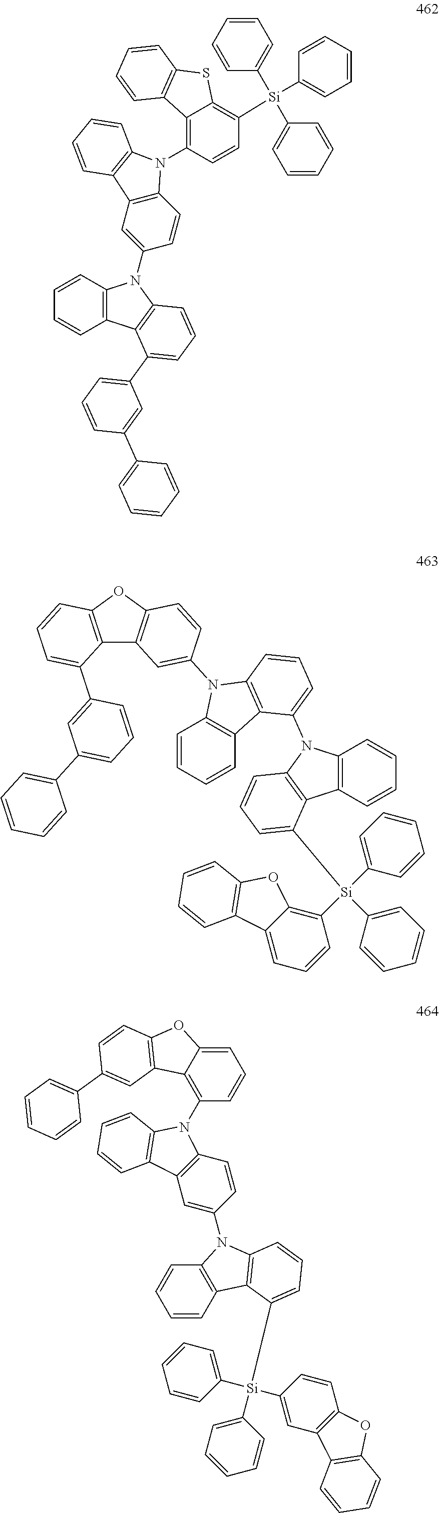 [SMILES: c1ccc(-c2cccc(-c3cccc4c3c3ccccc3n4-c3ccc4c(c3)c3ccccc3n4-c3ccc([Si](c4ccccc4)(c4ccccc4)c4ccccc4)c4sc5ccccc5c34)c2)cc1 ; c1ccc(-c2cccc(-c3cccc4oc5ccc(-n6c7ccccc7c7c(-n8c9ccccc9c9c([Si](c%10ccccc%10)(c%10ccccc%10)c%10cccc%11c%10oc%10ccccc%10%11)cccc98)cccc76)cc5c34)c2)cc1 ; c1ccc(-c2ccc3oc4cccc(-n5c6ccccc6c6cc(-n7c8ccccc8c8c([Si](c9ccccc9)(c9ccccc9)c9ccc%10oc%11ccccc%11c%10c9)cccc87)ccc65)c4c3c2)cc1]

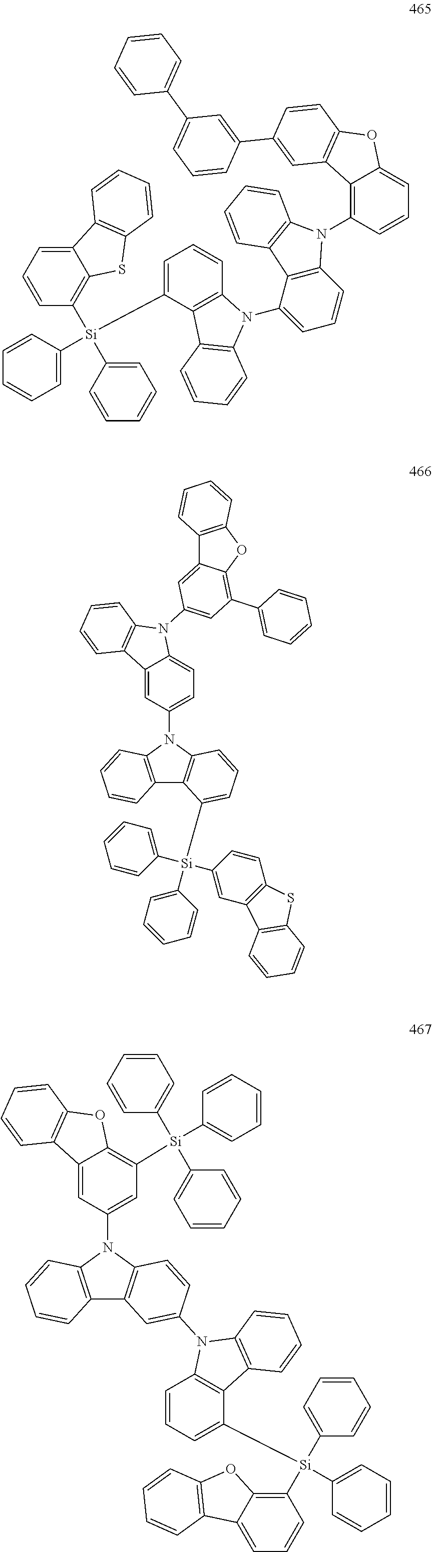 [SMILES: c1ccc(-c2cccc(-c3ccc4oc5cccc(-n6c7ccccc7c7c(-n8c9ccccc9c9c([Si](c%10ccccc%10)(c%10ccccc%10)c%10cccc%11c%10sc%10ccccc%10%11)cccc98)cccc76)c5c4c3)c2)cc1 ; c1ccc(-c2cc(-n3c4ccccc4c4cc(-n5c6ccccc6c6c([Si](c7ccccc7)(c7ccccc7)c7ccc8sc9ccccc9c8c7)cccc65)ccc43)cc3c2oc2ccccc23)cc1 ; c1ccc([Si](c2ccccc2)(c2ccccc2)c2cc(-n3c4ccccc4c4cc(-n5c6ccccc6c6c([Si](c7ccccc7)(c7ccccc7)c7cccc8c7oc7ccccc78)cccc65)ccc43)cc3c2oc2ccccc23)cc1]

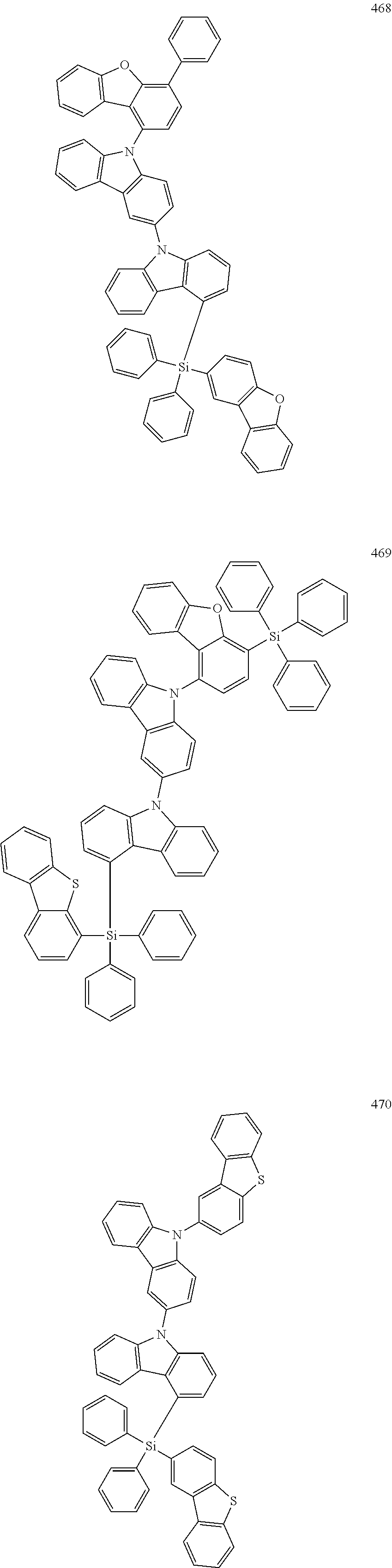 [SMILES: c1ccc(-c2ccc(-n3c4ccccc4c4cc(-n5c6ccccc6c6c([Si](c7ccccc7)(c7ccccc7)c7ccc8oc9ccccc9c8c7)cccc65)ccc43)c3c2oc2ccccc23)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7cccc8c7sc7ccccc78)cccc65)ccc43)c3c2oc2ccccc23)cc1 ; c1ccc([Si](c2ccccc2)(c2ccc3sc4ccccc4c3c2)c2cccc3c2c2ccccc2n3-c2ccc3c(c2)c2ccccc2n3-c2ccc3sc4ccccc4c3c2)cc1]

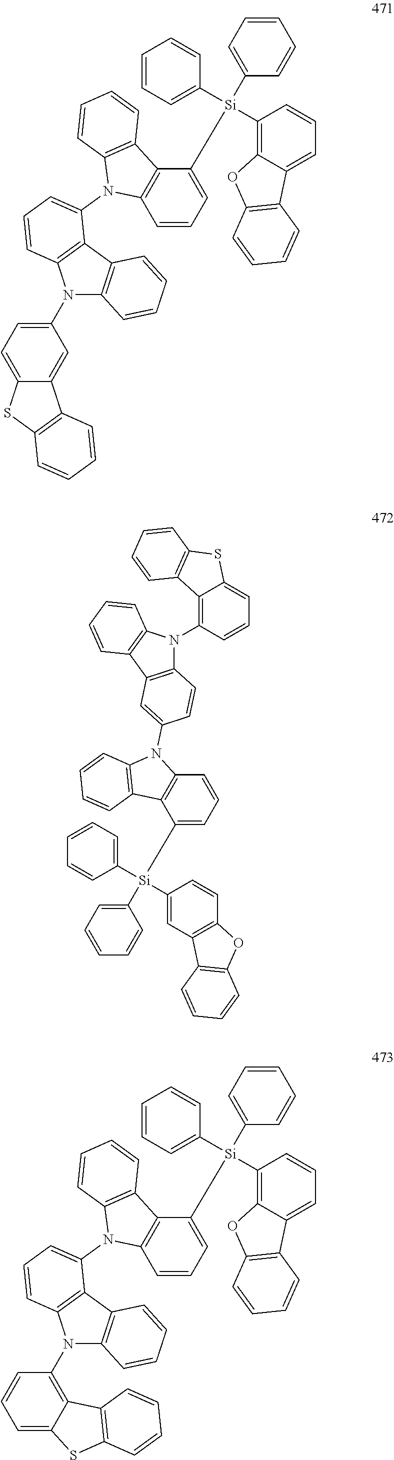 [SMILES: c1ccc([Si](c2ccccc2)(c2cccc3c2oc2ccccc23)c2cccc3c2c2ccccc2n3-c2cccc3c2c2ccccc2n3-c2ccc3sc4ccccc4c3c2)cc1 ; c1ccc([Si](c2ccccc2)(c2ccc3oc4ccccc4c3c2)c2cccc3c2c2ccccc2n3-c2ccc3c(c2)c2ccccc2n3-c2cccc3sc4ccccc4c23)cc1 ; c1ccc([Si](c2ccccc2)(c2cccc3c2oc2ccccc23)c2cccc3c2c2ccccc2n3-c2cccc3c2c2ccccc2n3-c2cccc3sc4ccccc4c23)cc1]

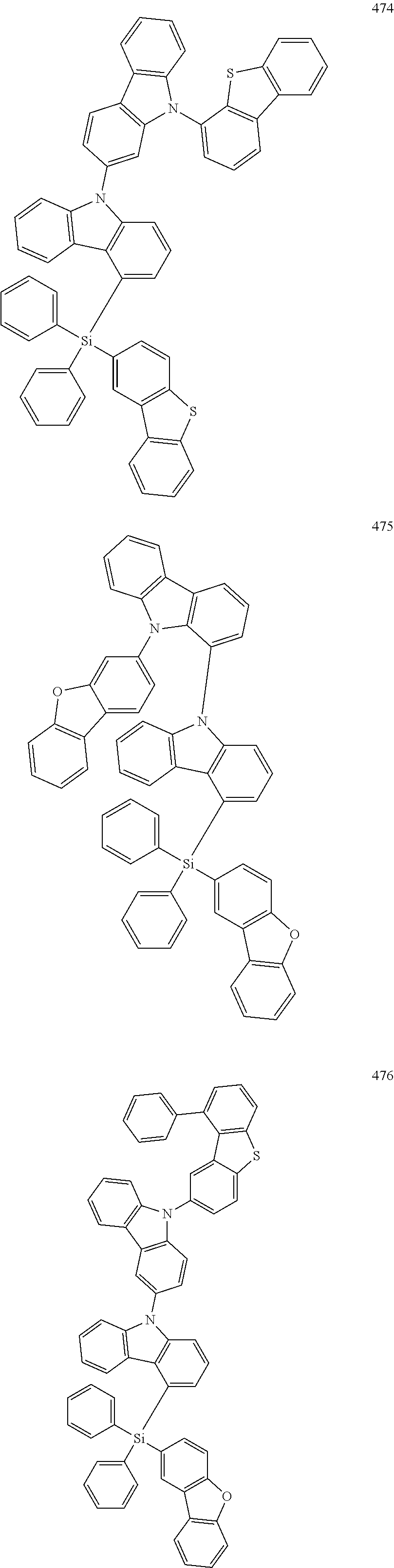

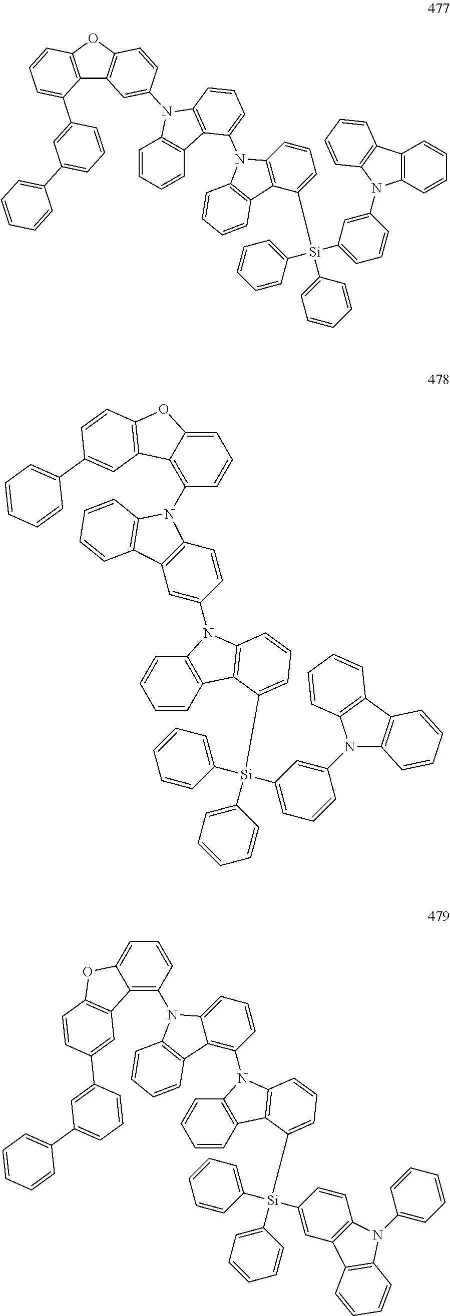 [SMILES: c1ccc(-c2cccc(-c3cccc4oc5ccc(-n6c7ccccc7c7c(-n8c9ccccc9c9c([Si](c%10ccccc%10)(c%10ccccc%10)c%10cccc(-n%11c%12ccccc%12c%12ccccc%12%11)c%10)cccc98)cccc76)cc5c34)c2)cc1 ; c1ccc(-c2ccc3oc4cccc(-n5c6ccccc6c6cc(-n7c8ccccc8c8c([Si](c9ccccc9)(c9ccccc9)c9cccc(-n%10c%11ccccc%11c%11ccccc%11%10)c9)cccc87)ccc65)c4c3c2)cc1 ; c1ccc(-c2cccc(-c3ccc4oc5cccc(-n6c7ccccc7c7c(-n8c9ccccc9c9c([Si](c%10ccccc%10)(c%10ccccc%10)c%10ccc%11c(c%10)c%10ccccc%10n%11-c%10ccccc%10)cccc98)cccc76)c5c4c3)c2)cc1]

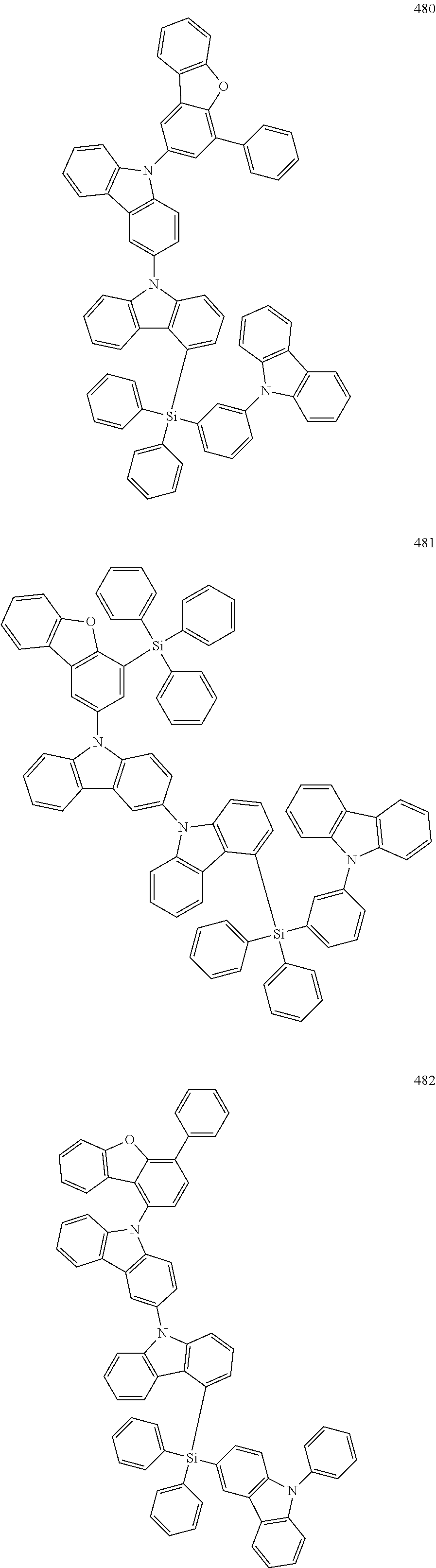 [SMILES: c1ccc(-c2cc(-n3c4ccccc4c4cc(-n5c6ccccc6c6c([Si](c7ccccc7)(c7ccccc7)c7cccc(-n8c9ccccc9c9ccccc98)c7)cccc65)ccc43)cc3c2oc2ccccc23)cc1 ; c1ccc([Si](c2ccccc2)(c2ccccc2)c2cc(-n3c4ccccc4c4cc(-n5c6ccccc6c6c([Si](c7ccccc7)(c7ccccc7)c7cccc(-n8c9ccccc9c9ccccc98)c7)cccc65)ccc43)cc3c2oc2ccccc23)cc1 ; c1ccc(-c2ccc(-n3c4ccccc4c4cc(-n5c6ccccc6c6c([Si](c7ccccc7)(c7ccccc7)c7ccc8c(c7)c7ccccc7n8-c7ccccc7)cccc65)ccc43)c3c2oc2ccccc23)cc1]

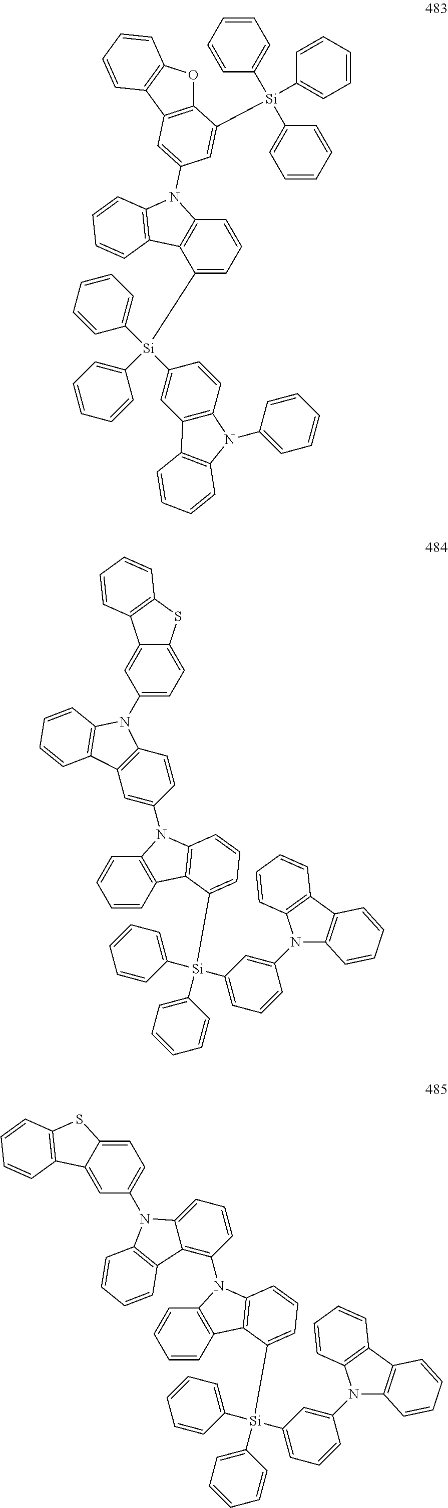 [SMILES: c1ccc(-n2c3ccccc3c3cc([Si](c4ccccc4)(c4ccccc4)c4cccc5c4c4ccccc4n5-c4cc([Si](c5ccccc5)(c5ccccc5)c5ccccc5)c5oc6ccccc6c5c4)ccc32)cc1 ; c1ccc([Si](c2ccccc2)(c2cccc(-n3c4ccccc4c4ccccc43)c2)c2cccc3c2c2ccccc2n3-c2ccc3c(c2)c2ccccc2n3-c2ccc3sc4ccccc4c3c2)cc1 ; c1ccc([Si](c2ccccc2)(c2cccc(-n3c4ccccc4c4ccccc43)c2)c2cccc3c2c2ccccc2n3-c2cccc3c2c2ccccc2n3-c2ccc3sc4ccccc4c3c2)cc1]

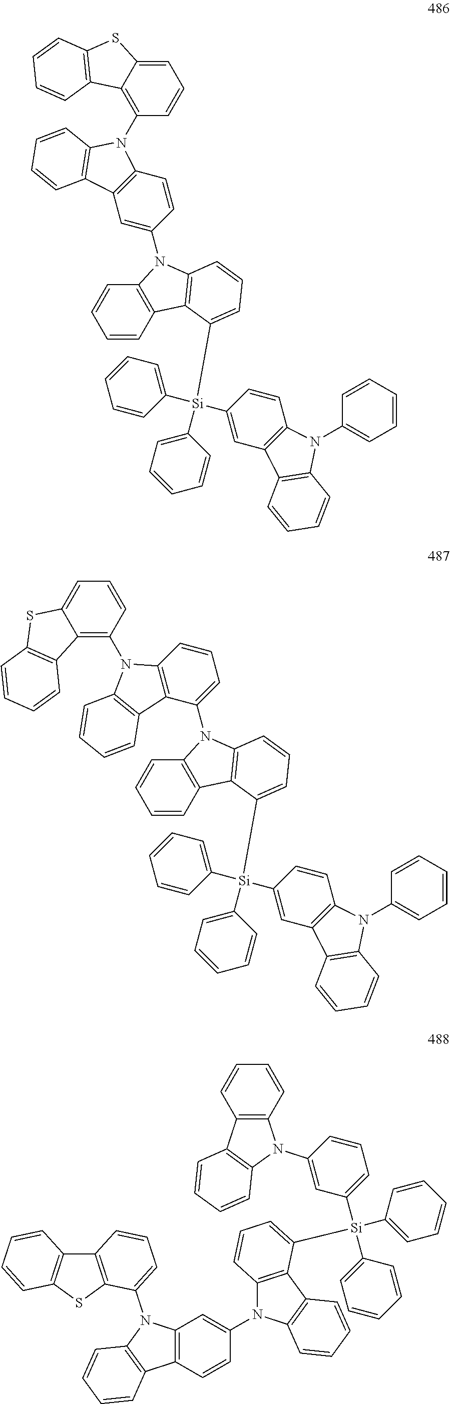 [SMILES: c1ccc(-n2c3ccccc3c3cc([Si](c4ccccc4)(c4ccccc4)c4cccc5c4c4ccccc4n5-c4ccc5c(c4)c4ccccc4n5-c4cccc5sc6ccccc6c45)ccc32)cc1 ; c1ccc(-n2c3ccccc3c3cc([Si](c4ccccc4)(c4ccccc4)c4cccc5c4c4ccccc4n5-c4cccc5c4c4ccccc4n5-c4cccc5sc6ccccc6c45)ccc32)cc1 ; c1ccc([Si](c2ccccc2)(c2cccc(-n3c4ccccc4c4ccccc43)c2)c2cccc3c2c2ccccc2n3-c2ccc3c4ccccc4n(-c4cccc5c4sc4ccccc45)c3c2)cc1]

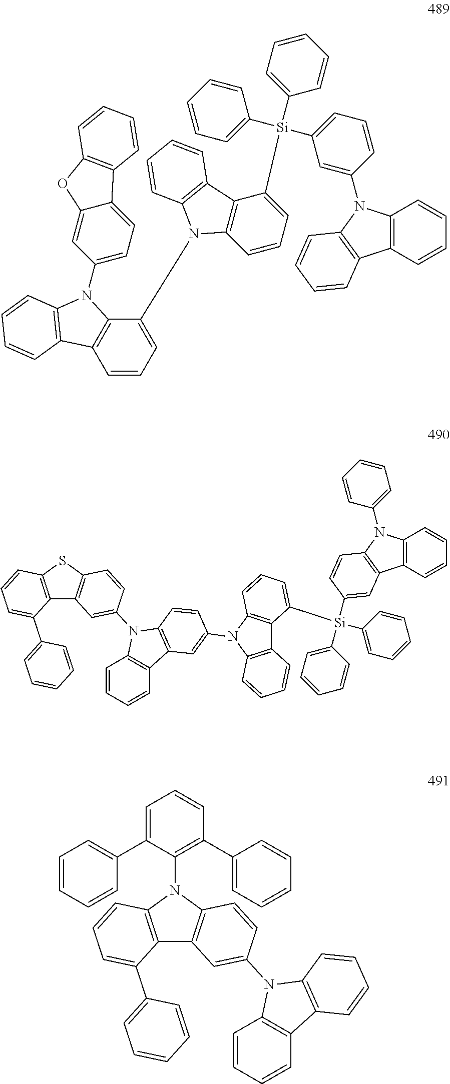 [SMILES: c1ccc([Si](c2ccccc2)(c2cccc(-n3c4ccccc4c4ccccc43)c2)c2cccc3c2c2ccccc2n3-c2cccc3c4ccccc4n(-c4ccc5c(c4)oc4ccccc45)c23)cc1 ; c1ccc(-c2cccc3sc4ccc(-n5c6ccccc6c6cc(-n7c8ccccc8c8c([Si](c9ccccc9)(c9ccccc9)c9ccc%10c(c9)c9ccccc9n%10-c9ccccc9)cccc87)ccc65)cc4c23)cc1 ; c1ccc(-c2cccc(-c3ccccc3)c2-n2c3ccc(-n4c5ccccc5c5ccccc54)cc3c3c(-c4ccccc4)cccc32)cc1]

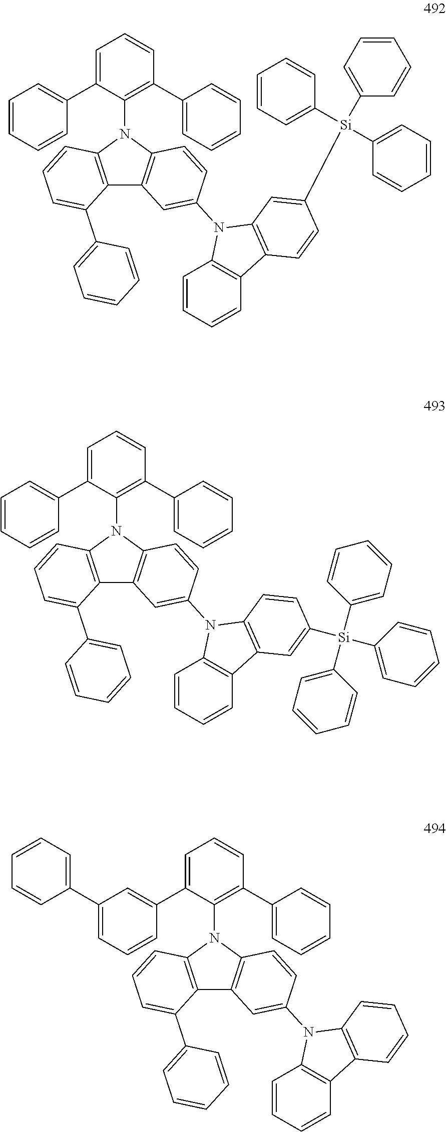 [SMILES: c1ccc(-c2cccc(-c3ccccc3)c2-n2c3ccc(-n4c5ccccc5c5ccc([Si](c6ccccc6)(c6ccccc6)c6ccccc6)cc54)cc3c3c(-c4ccccc4)cccc32)cc1 ; c1ccc(-c2cccc(-c3ccccc3)c2-n2c3ccc(-n4c5ccccc5c5cc([Si](c6ccccc6)(c6ccccc6)c6ccccc6)ccc54)cc3c3c(-c4ccccc4)cccc32)cc1 ; c1ccc(-c2cccc(-c3cccc(-c4ccccc4)c3-n3c4ccc(-n5c6ccccc6c6ccccc65)cc4c4c(-c5ccccc5)cccc43)c2)cc1]

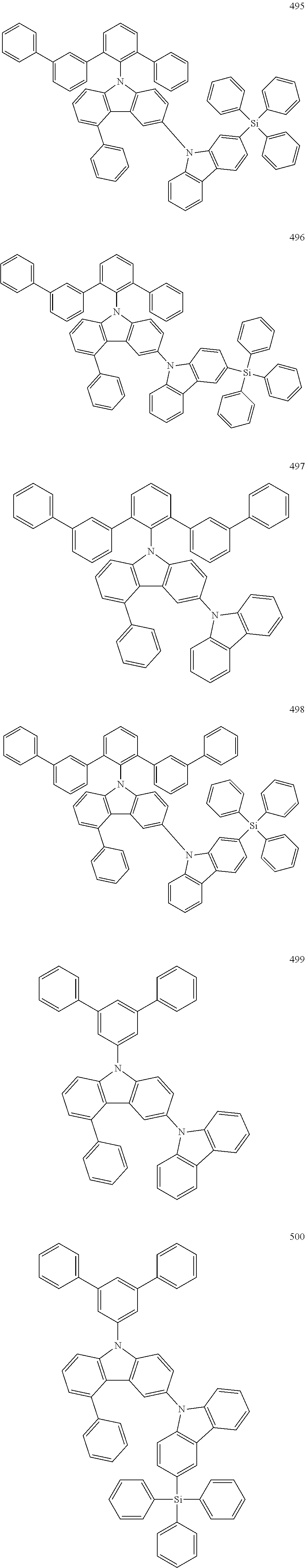

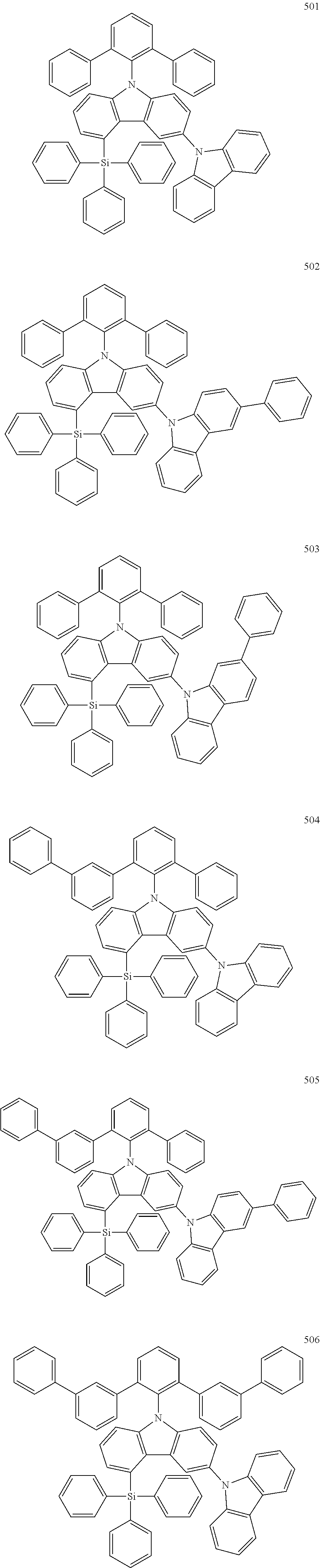

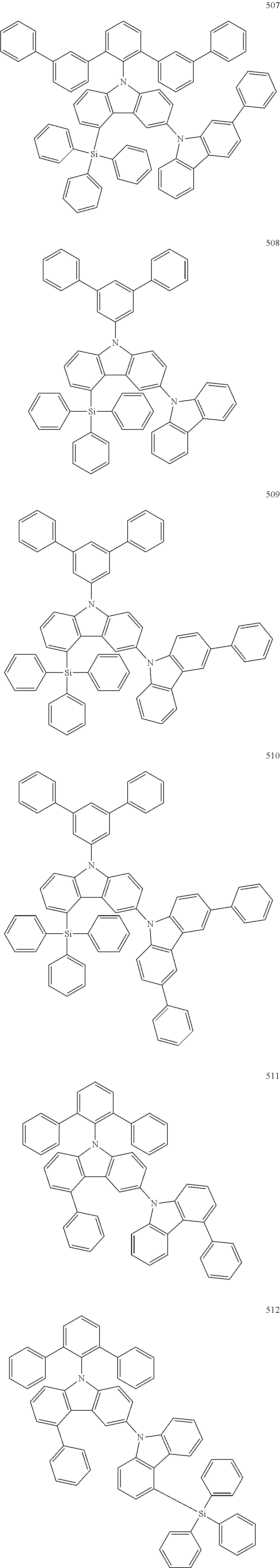

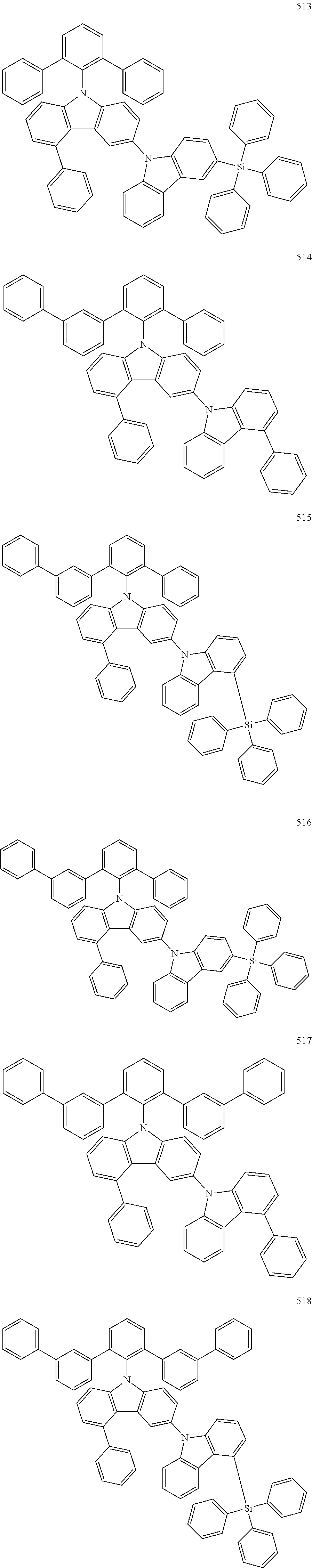

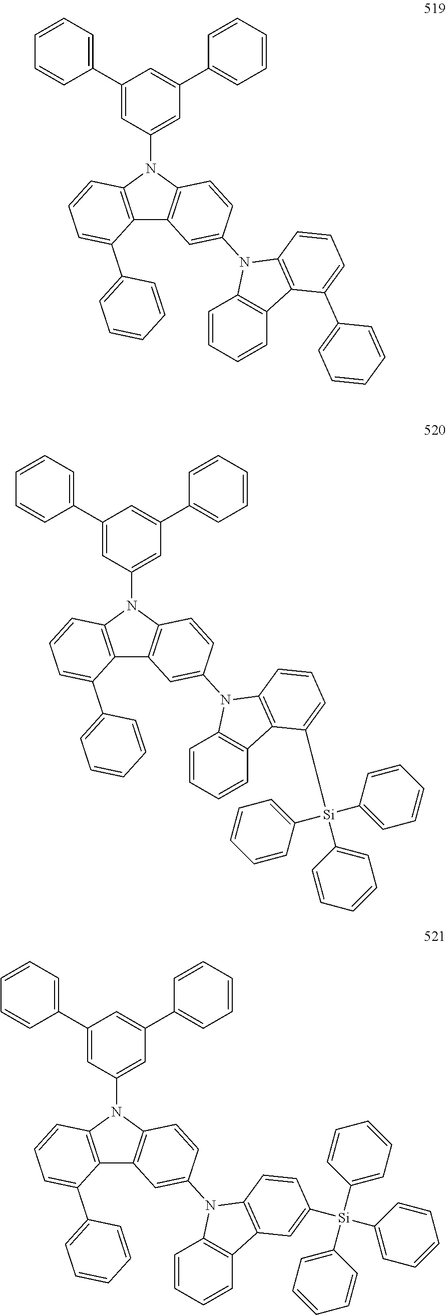 [SMILES: c1ccc(-c2cc(-c3ccccc3)cc(-n3c4ccc(-n5c6ccccc6c6c(-c7ccccc7)cccc65)cc4c4c(-c5ccccc5)cccc43)c2)cc1 ; c1ccc(-c2cc(-c3ccccc3)cc(-n3c4ccc(-n5c6ccccc6c6c([Si](c7ccccc7)(c7ccccc7)c7ccccc7)cccc65)cc4c4c(-c5ccccc5)cccc43)c2)cc1 ; c1ccc(-c2cc(-c3ccccc3)cc(-n3c4ccc(-n5c6ccccc6c6cc([Si](c7ccccc7)(c7ccccc7)c7ccccc7)ccc65)cc4c4c(-c5ccccc5)cccc43)c2)cc1]

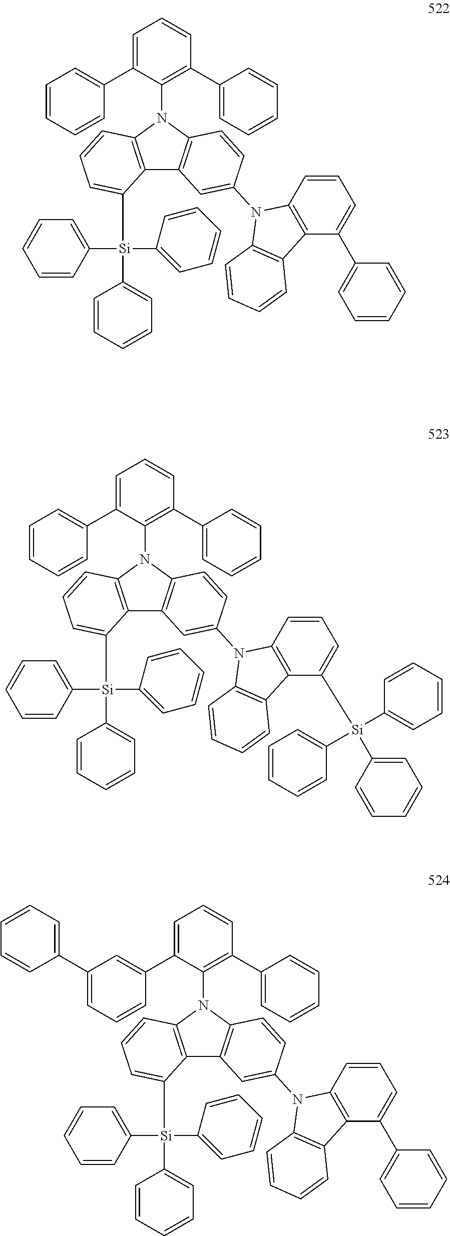 [SMILES: c1ccc(-c2cccc(-c3ccccc3)c2-n2c3ccc(-n4c5ccccc5c5c(-c6ccccc6)cccc54)cc3c3c([Si](c4ccccc4)(c4ccccc4)c4ccccc4)cccc32)cc1 ; c1ccc(-c2cccc(-c3ccccc3)c2-n2c3ccc(-n4c5ccccc5c5c([Si](c6ccccc6)(c6ccccc6)c6ccccc6)cccc54)cc3c3c([Si](c4ccccc4)(c4ccccc4)c4ccccc4)cccc32)cc1 ; c1ccc(-c2cccc(-c3cccc(-c4ccccc4)c3-n3c4ccc(-n5c6ccccc6c6c(-c7ccccc7)cccc65)cc4c4c([Si](c5ccccc5)(c5ccccc5)c5ccccc5)cccc43)c2)cc1]

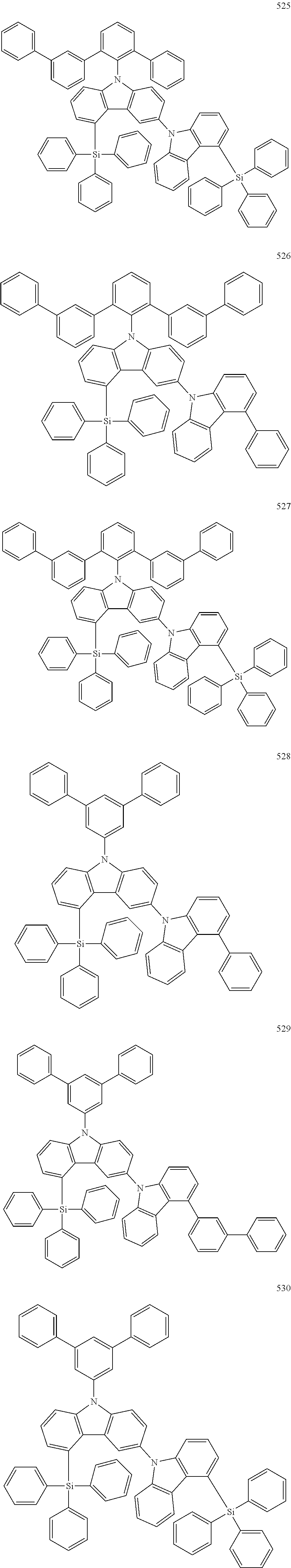

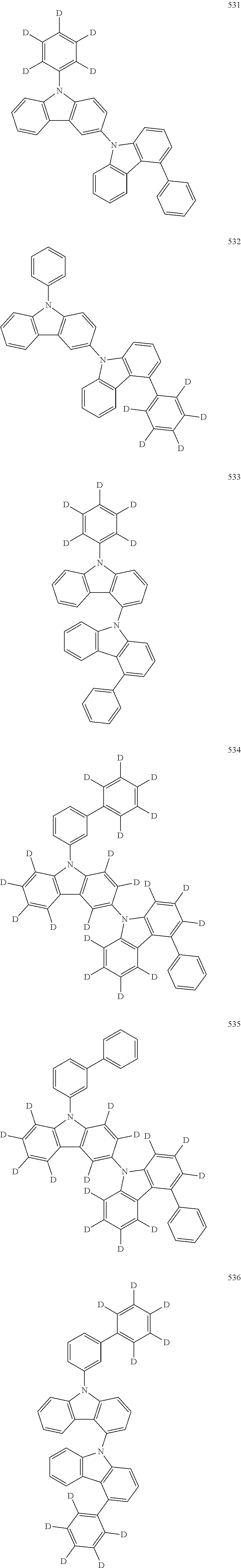

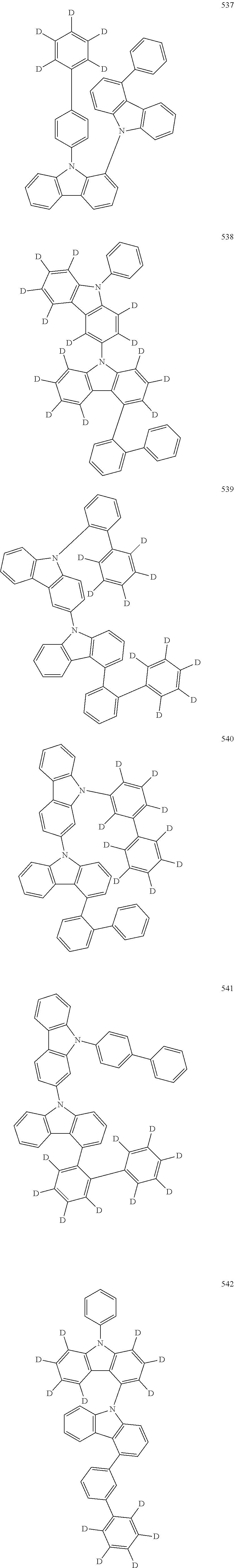

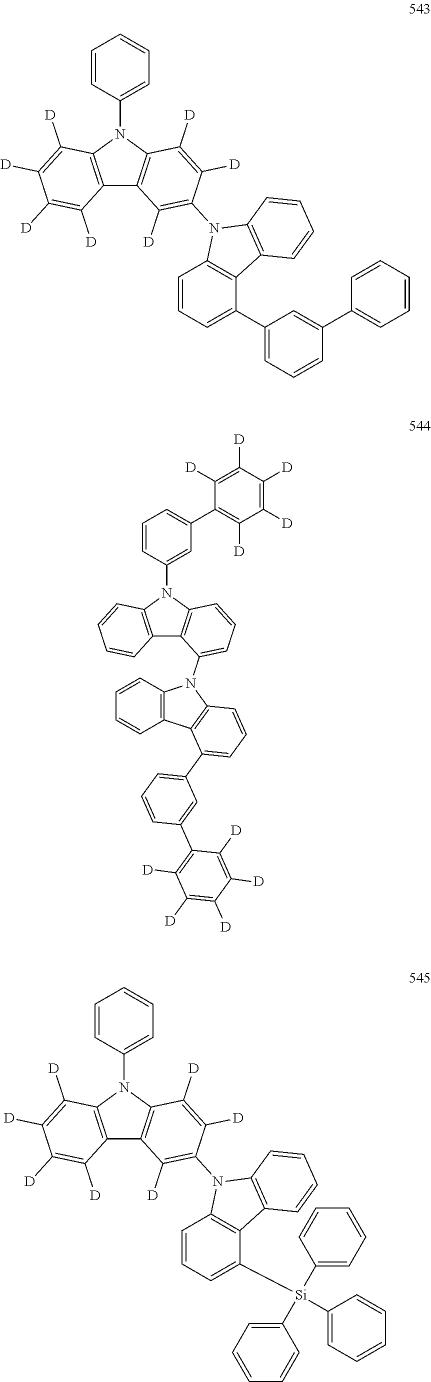 [SMILES: [2H]c1c([2H])c([2H])c2c(c1[2H])c1c([2H])c(-n3c4ccccc4c4c(-c5cccc(-c6ccccc6)c5)cccc43)c([2H])c([2H])c1n2-c1ccccc1 ; [2H]c1c([2H])c([2H])c(-c2cccc(-c3cccc4c3c3ccccc3n4-c3cccc4c3c3ccccc3n4-c3cccc(-c4c([2H])c([2H])c([2H])c([2H])c4[2H])c3)c2)c([2H])c1[2H] ; [2H]c1c([2H])c([2H])c2c(c1[2H])c1c([2H])c(-n3c4ccccc4c4c([Si](c5ccccc5)(c5ccccc5)c5ccccc5)cccc43)c([2H])c([2H])c1n2-c1ccccc1]

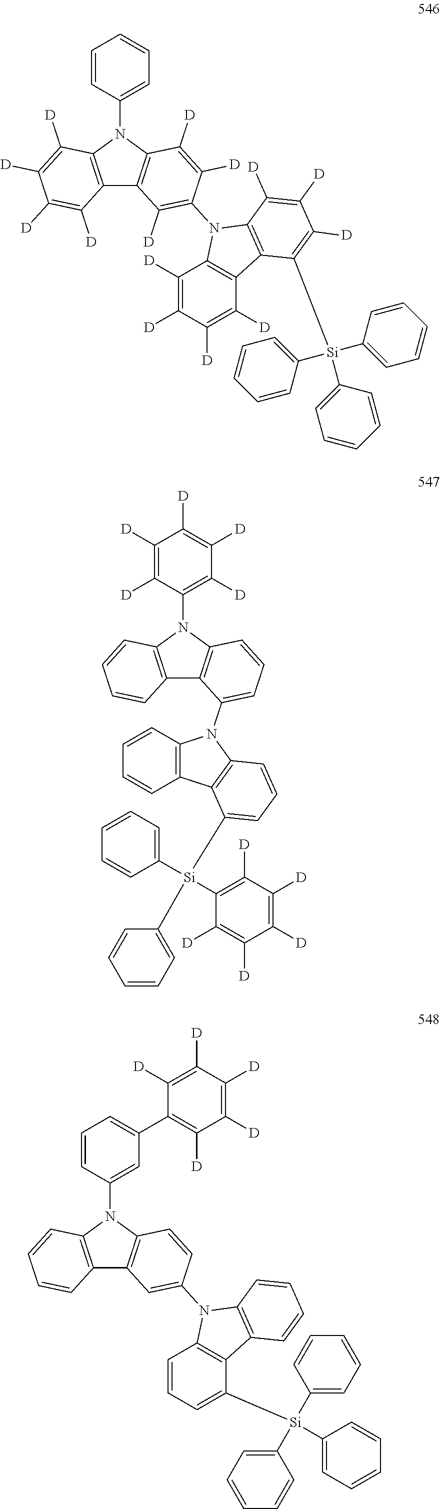 [SMILES: [2H]c1c([2H])c([2H])c2c(c1[2H])c1c([2H])c(-n3c4c([2H])c([2H])c([2H])c([2H])c4c4c([Si](c5ccccc5)(c5ccccc5)c5ccccc5)c([2H])c([2H])c([2H])c43)c([2H])c([2H])c1n2-c1ccccc1 ; [2H]c1c([2H])c([2H])c(-n2c3ccccc3c3c(-n4c5ccccc5c5c([Si](c6ccccc6)(c6ccccc6)c6c([2H])c([2H])c([2H])c([2H])c6[2H])cccc54)cccc32)c([2H])c1[2H] ; [2H]c1c([2H])c([2H])c(-c2cccc(-n3c4ccccc4c4cc(-n5c6ccccc6c6c([Si](c7ccccc7)(c7ccccc7)c7ccccc7)cccc65)ccc43)c2)c([2H])c1[2H]]

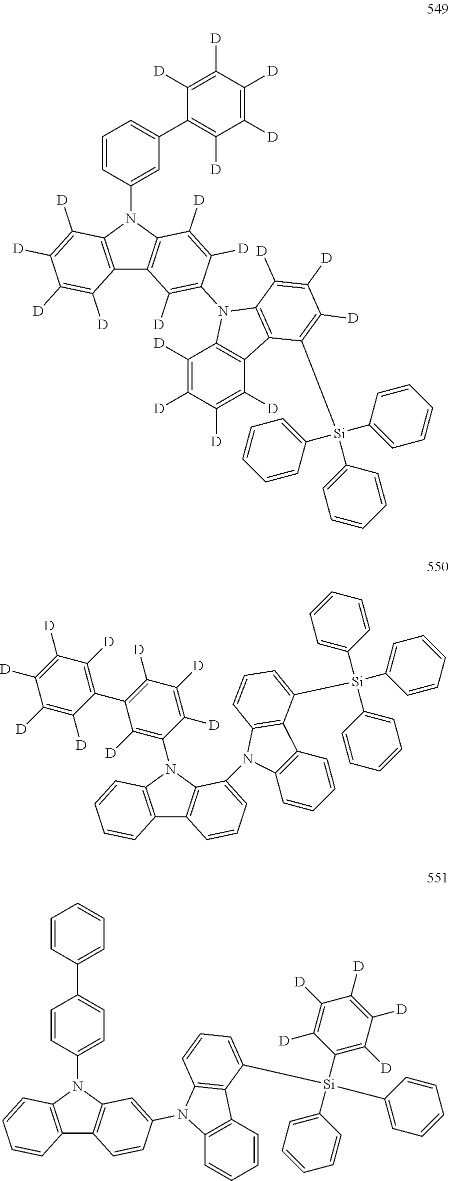 [SMILES: [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c([Si](c7ccccc7)(c7ccccc7)c7ccccc7)c([2H])c([2H])c([2H])c65)c([2H])c([2H])c43)c2)c([2H])c1[2H] ; [2H]c1c([2H])c([2H])c(-c2c([2H])c([2H])c([2H])c(-n3c4ccccc4c4cccc(-n5c6ccccc6c6c([Si](c7ccccc7)(c7ccccc7)c7ccccc7)cccc65)c43)c2[2H])c([2H])c1[2H] ; [2H]c1c([2H])c([2H])c([Si](c2ccccc2)(c2ccccc2)c2cccc3c2c2ccccc2n3-c2ccc3c4ccccc4n(-c4ccc(-c5ccccc5)cc4)c3c2)c([2H])c1[2H]]

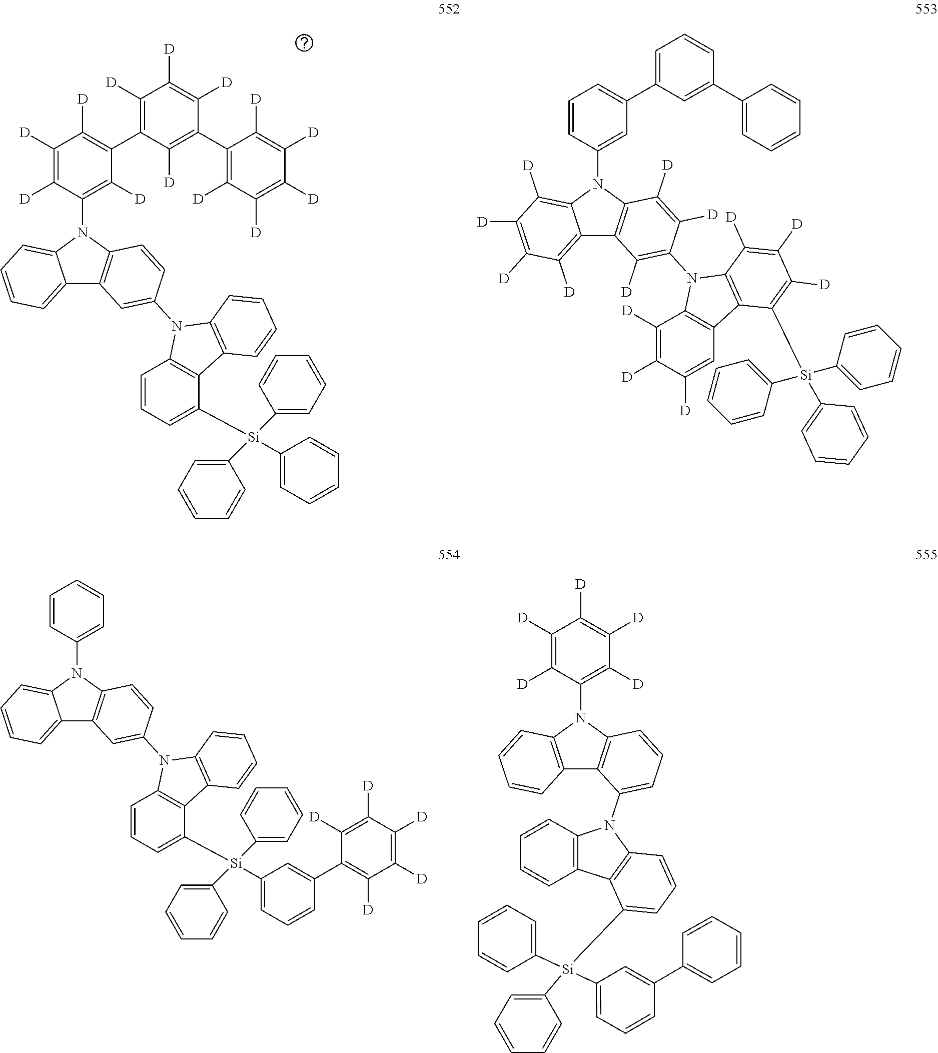

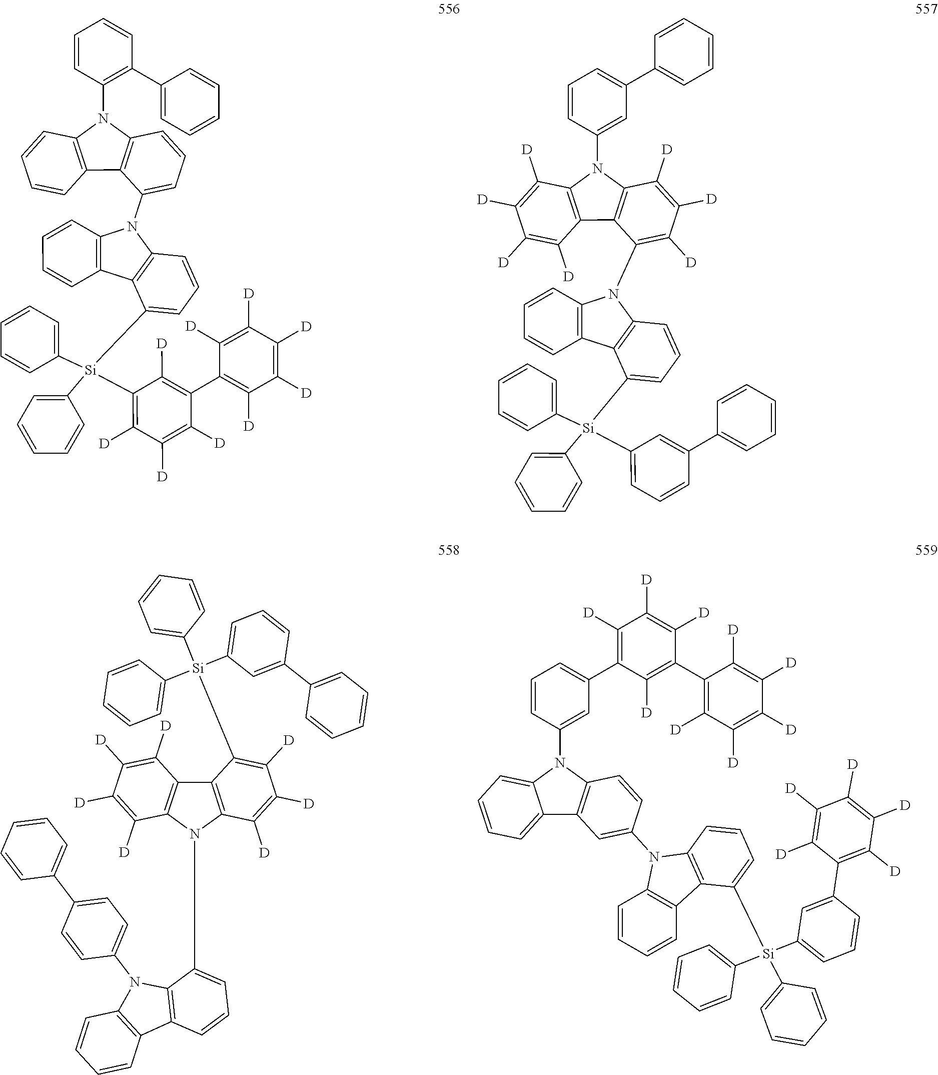

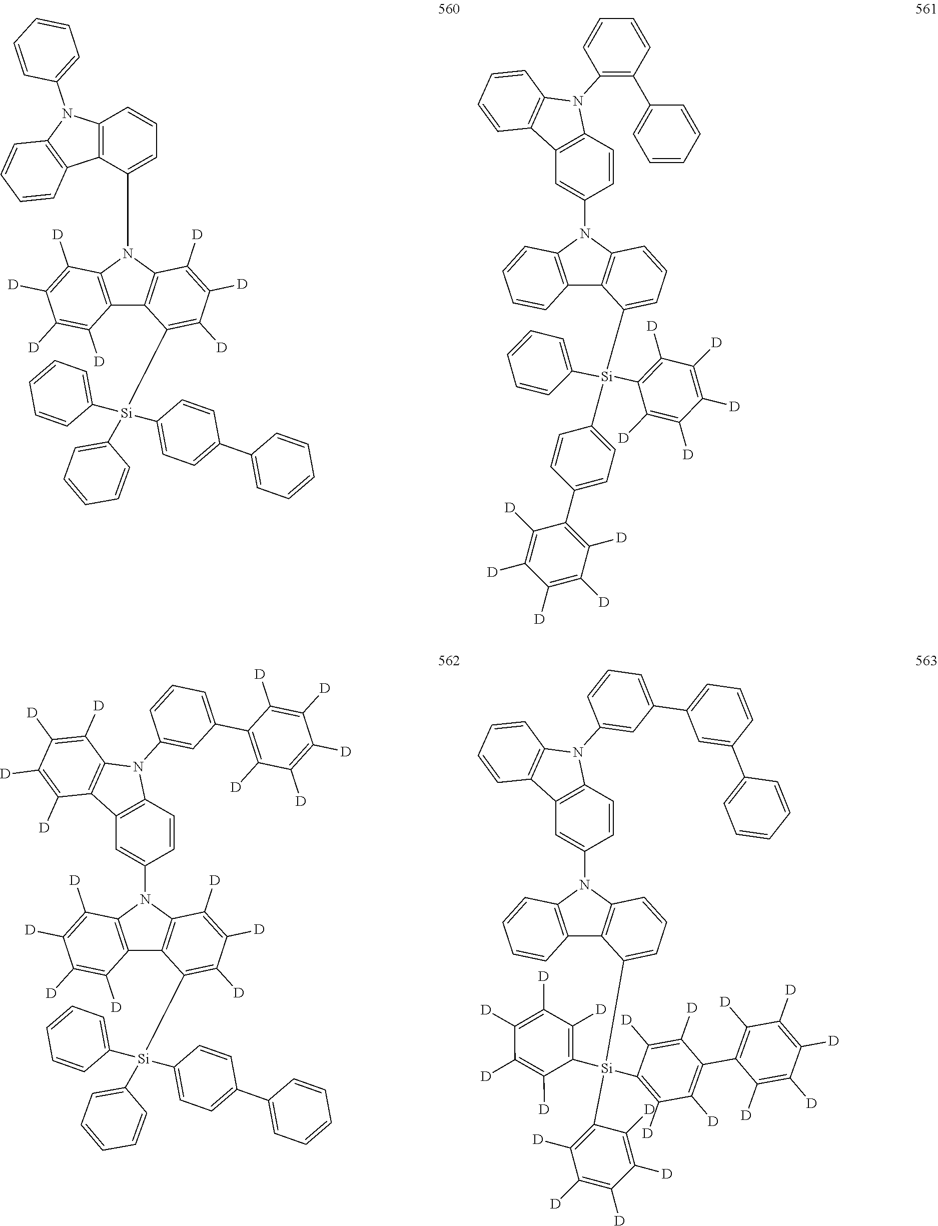

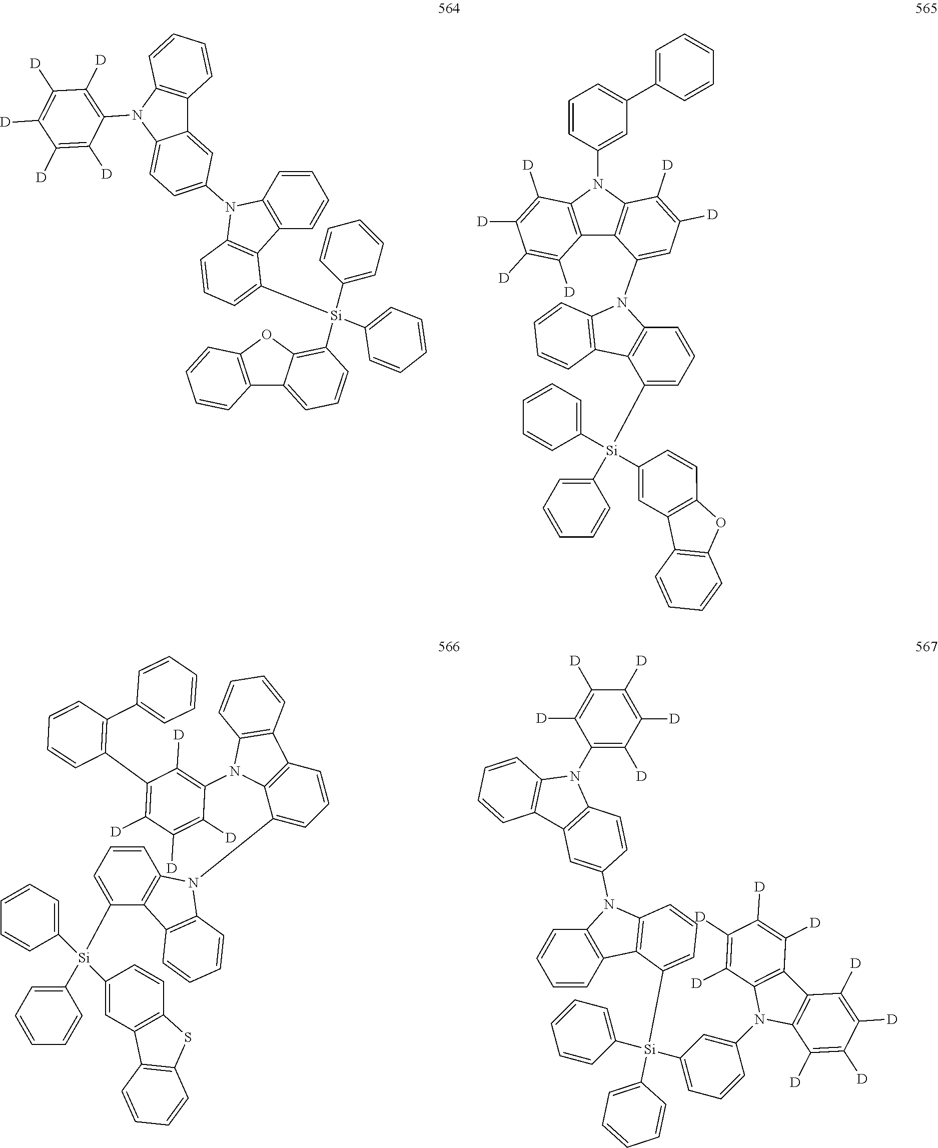

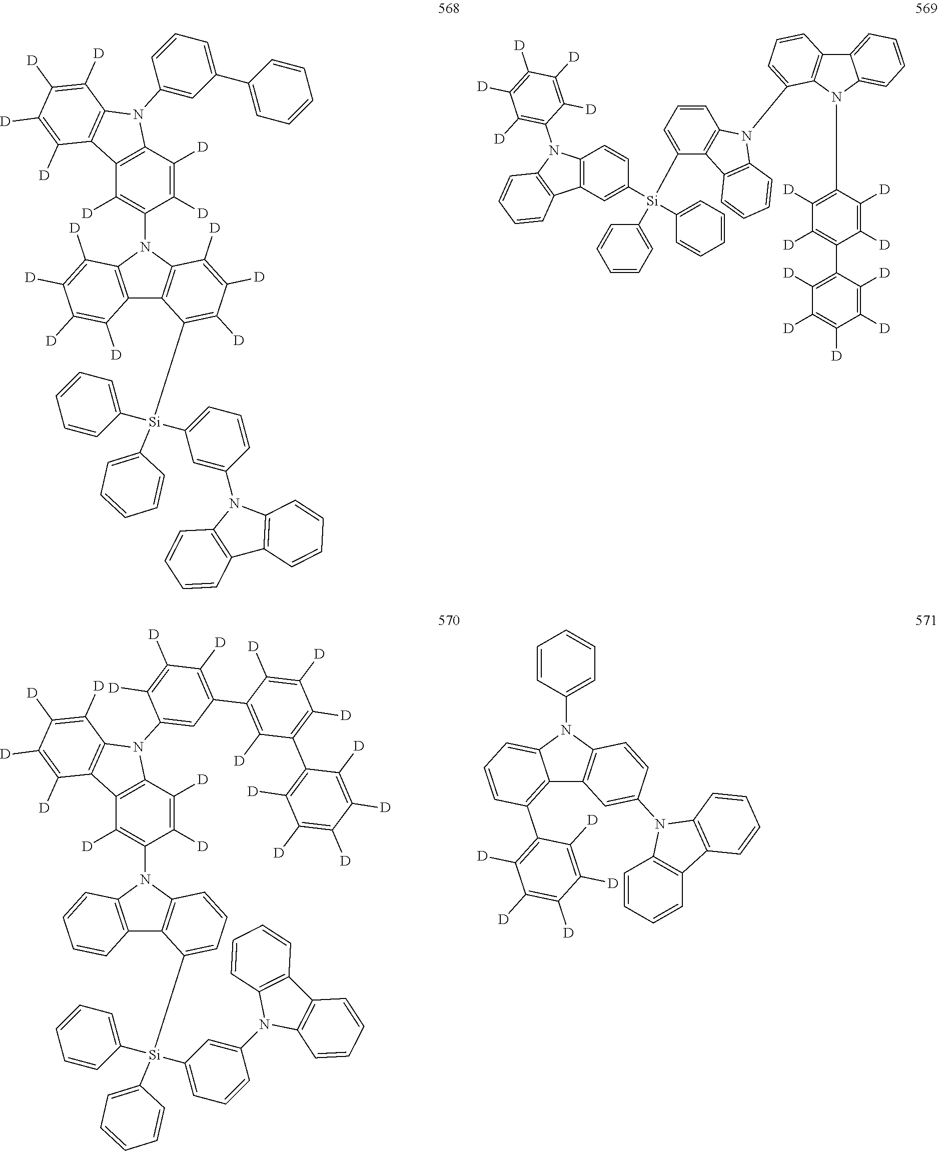

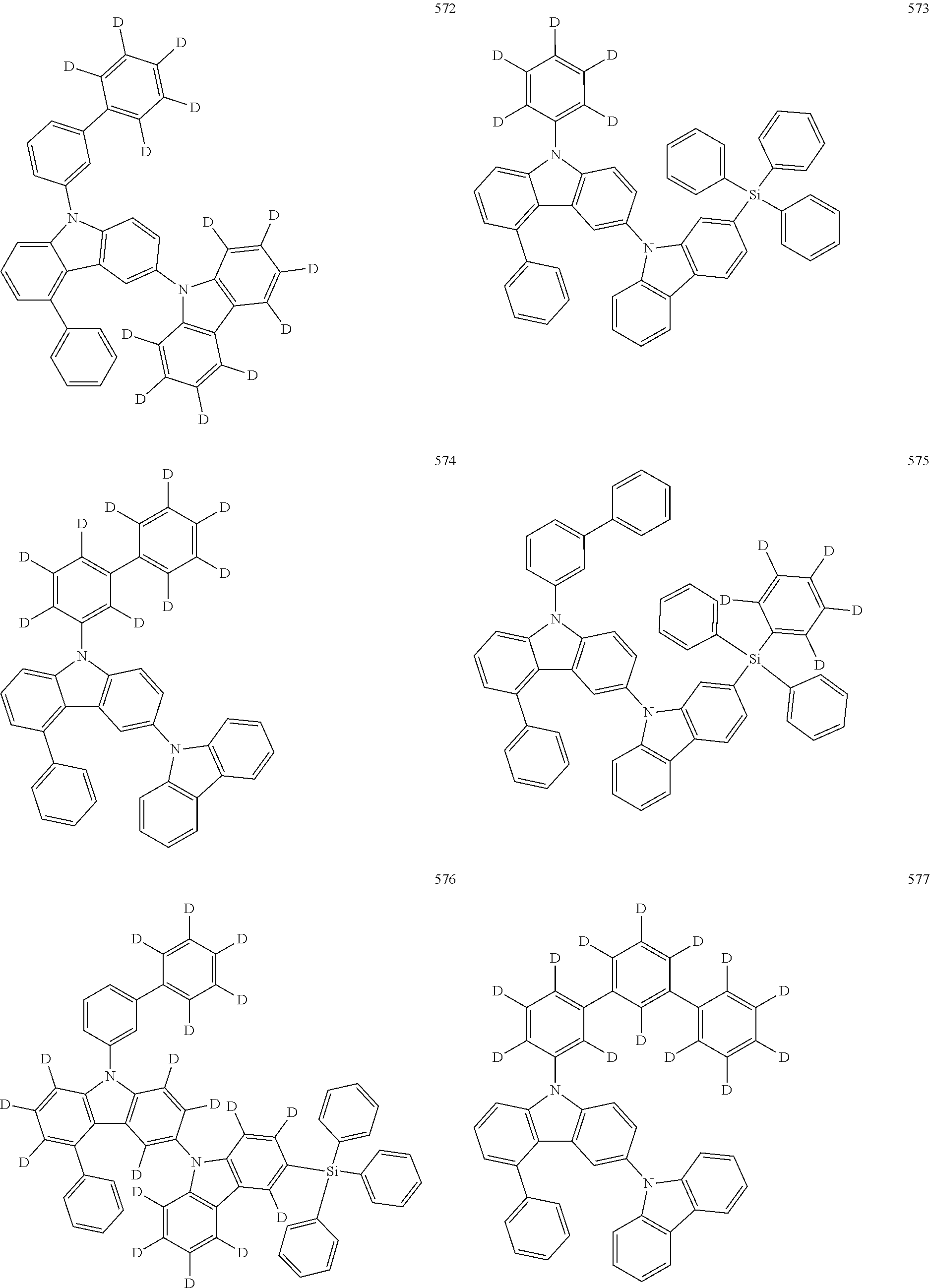

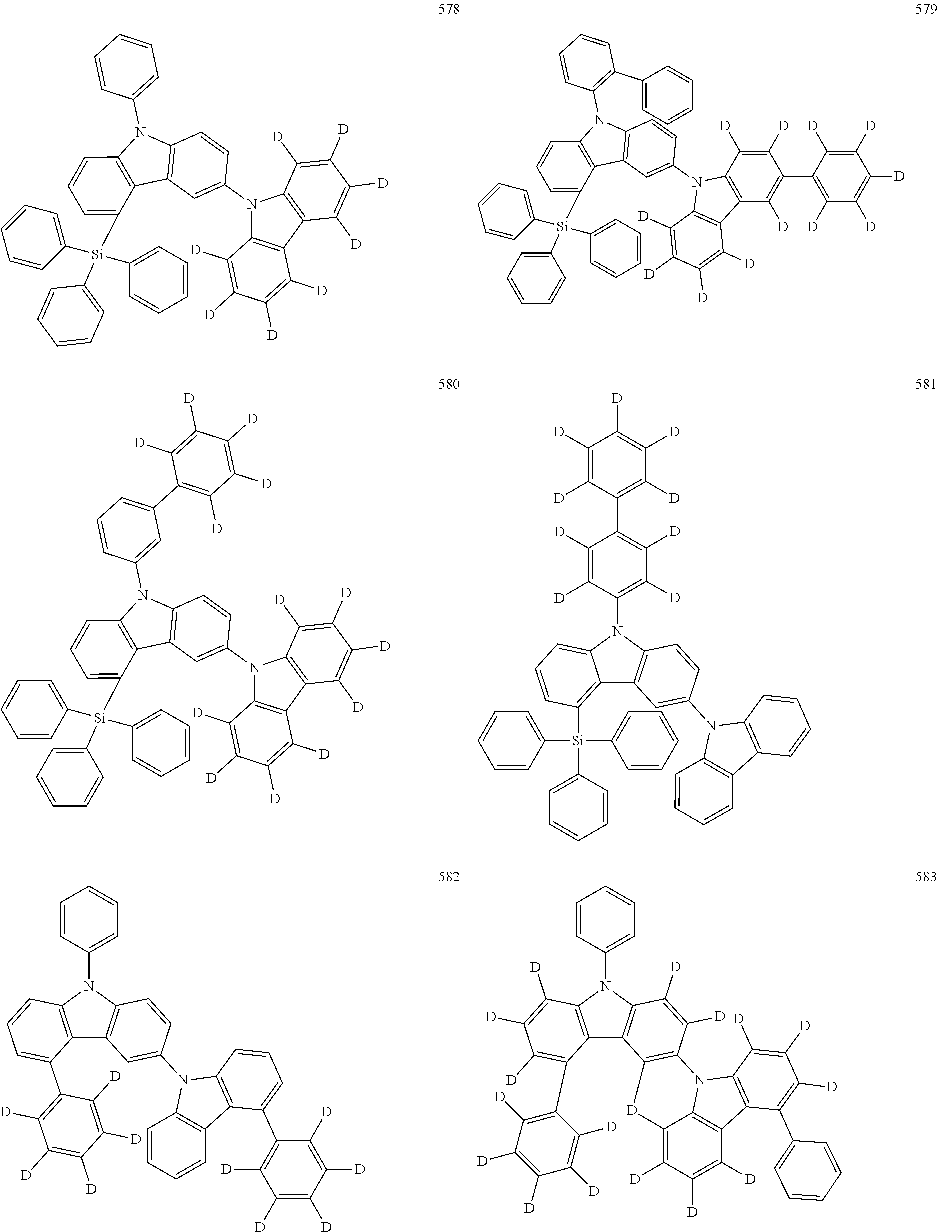

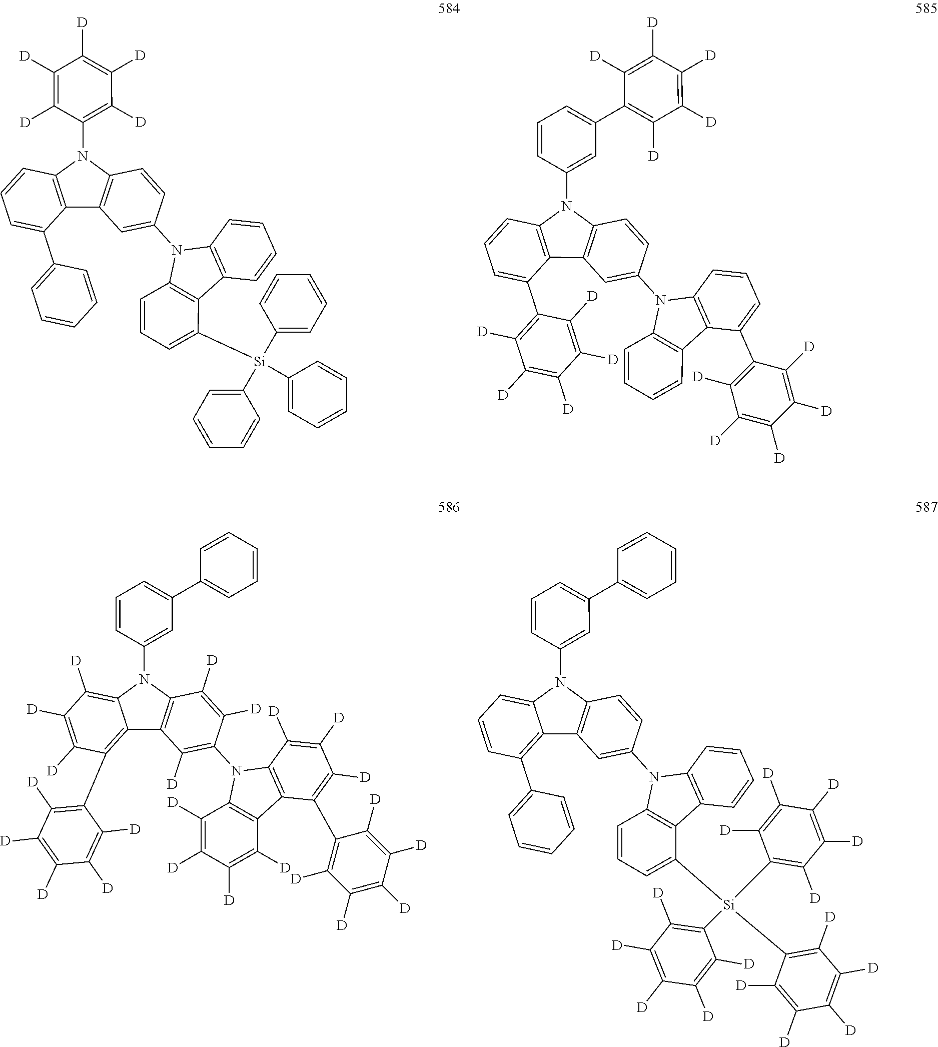

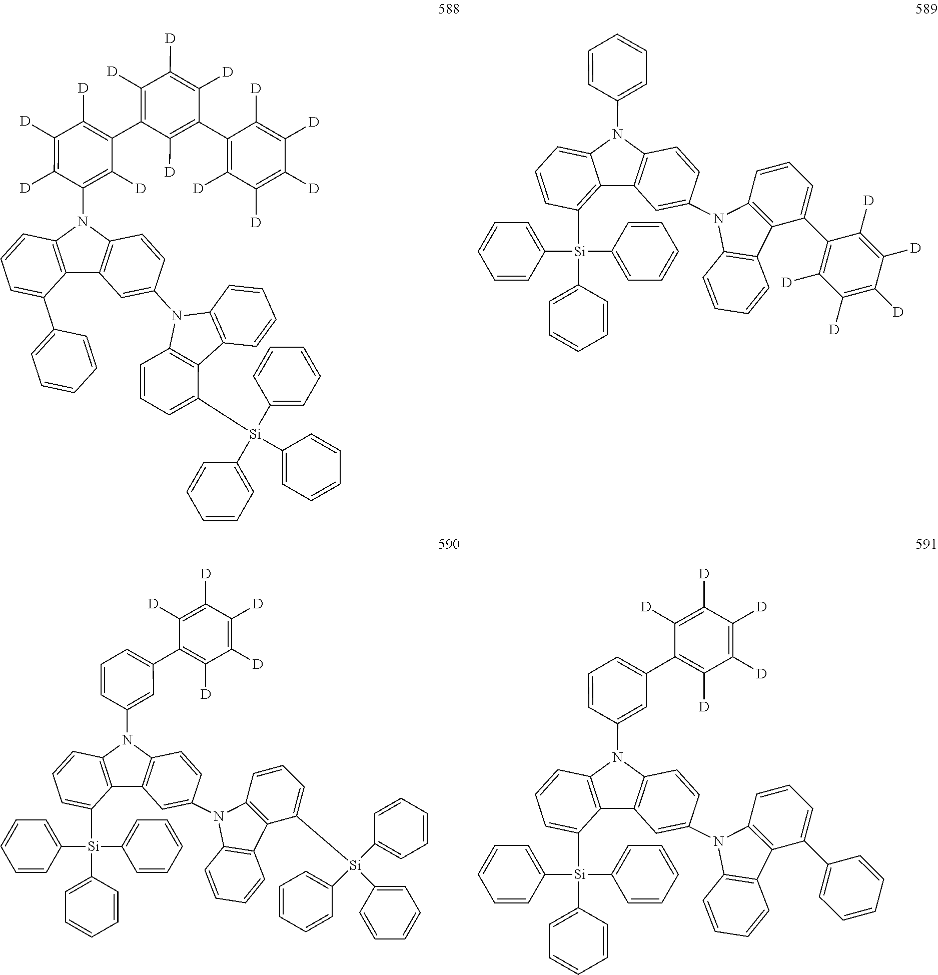

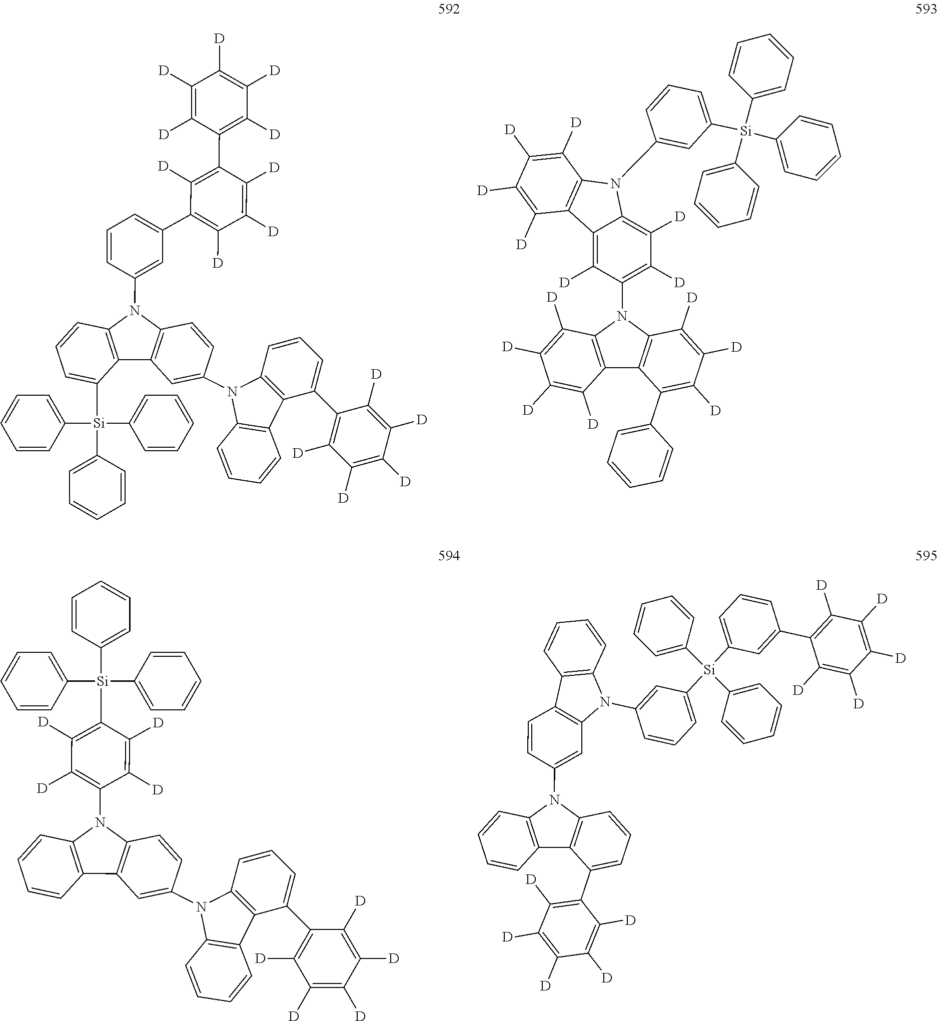

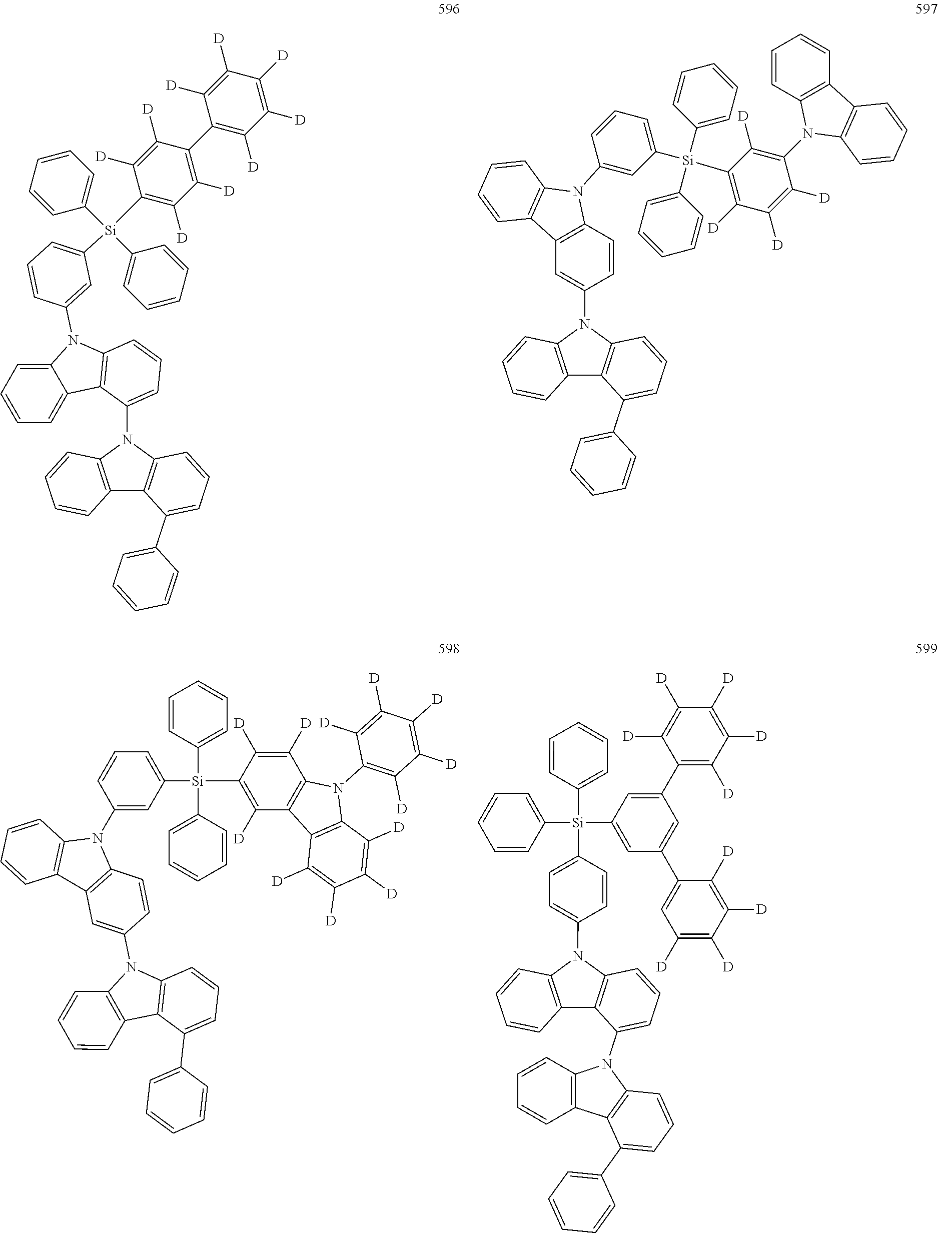

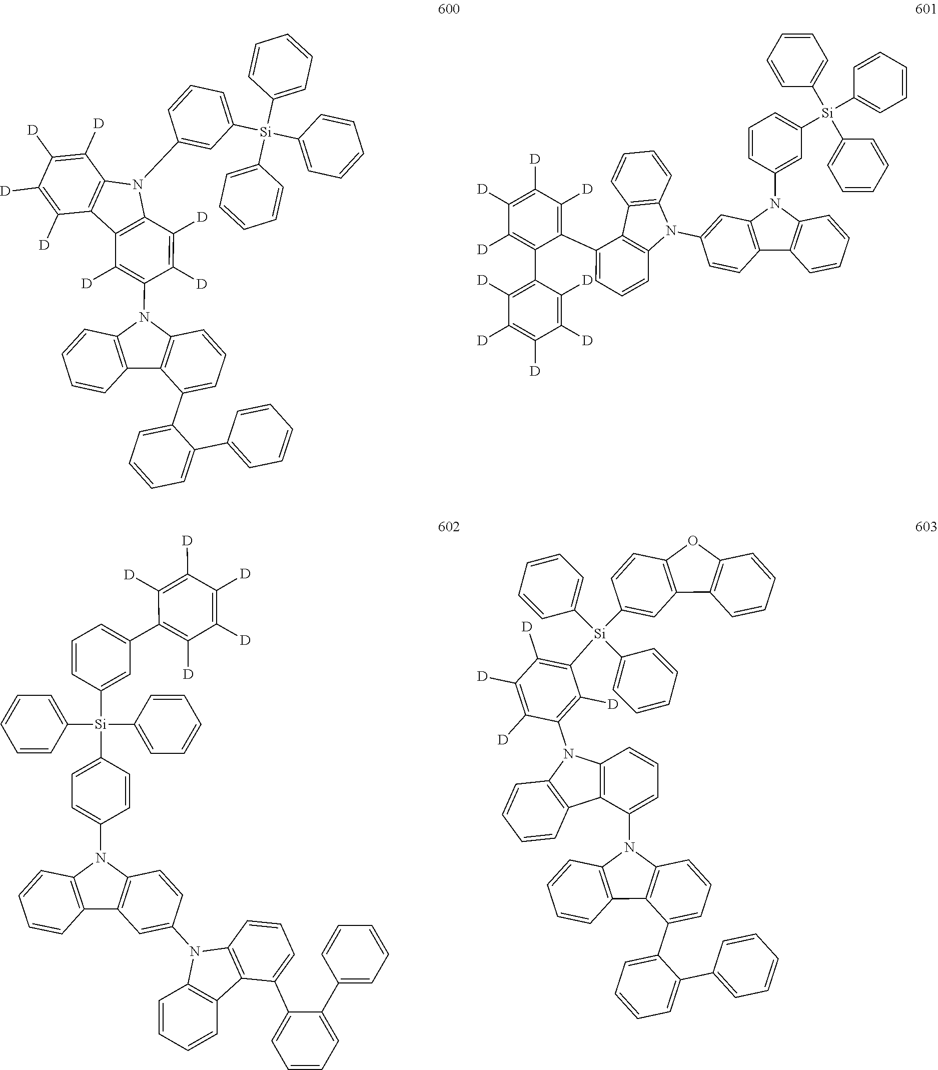

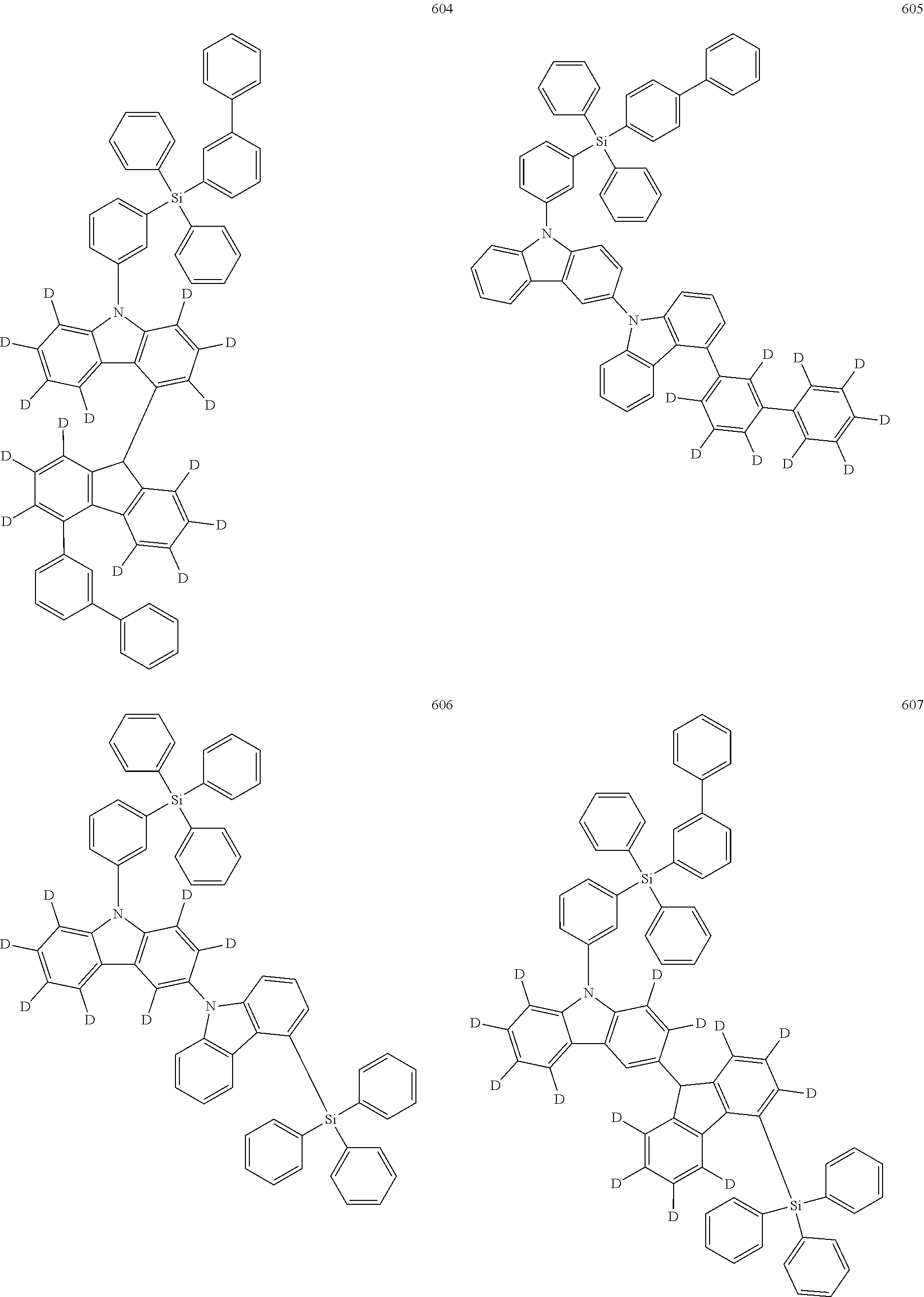

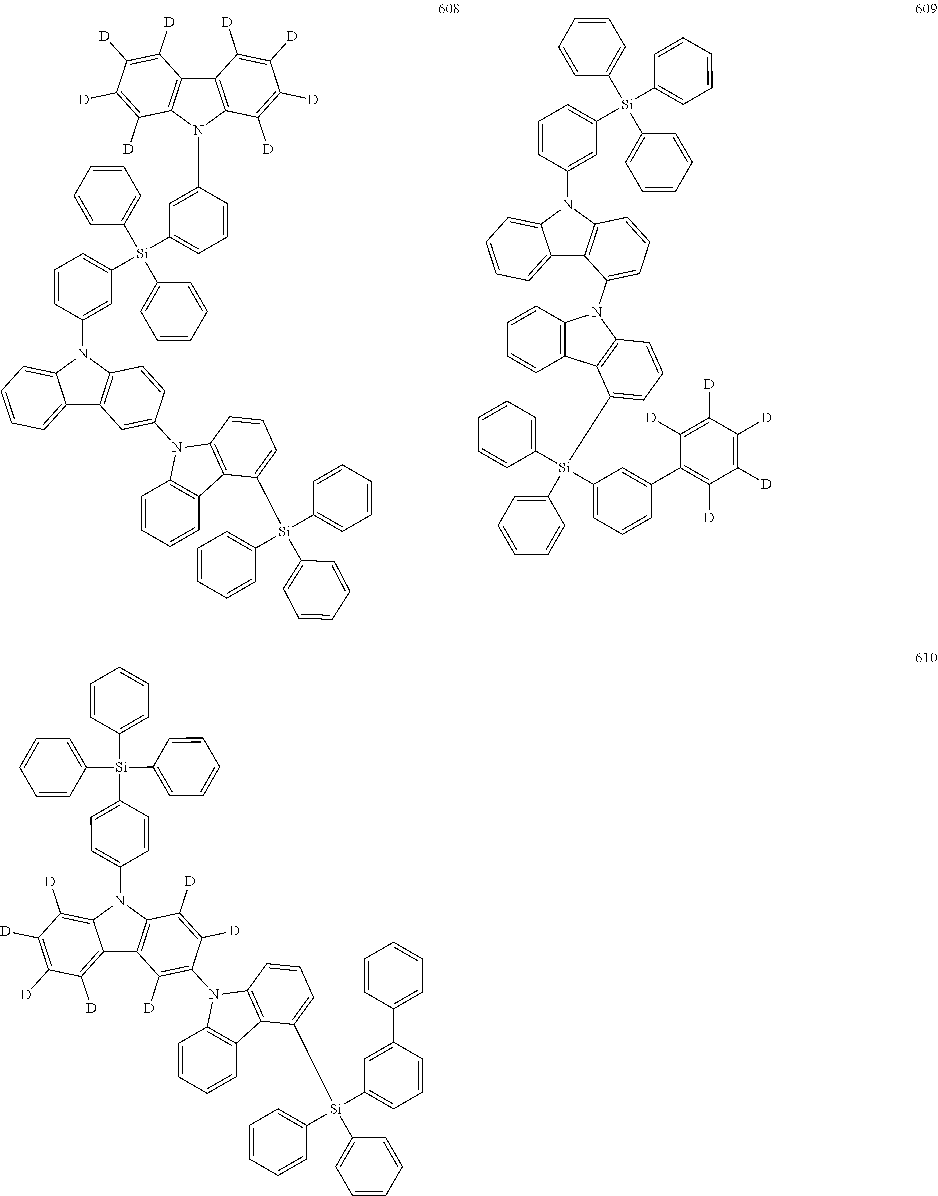

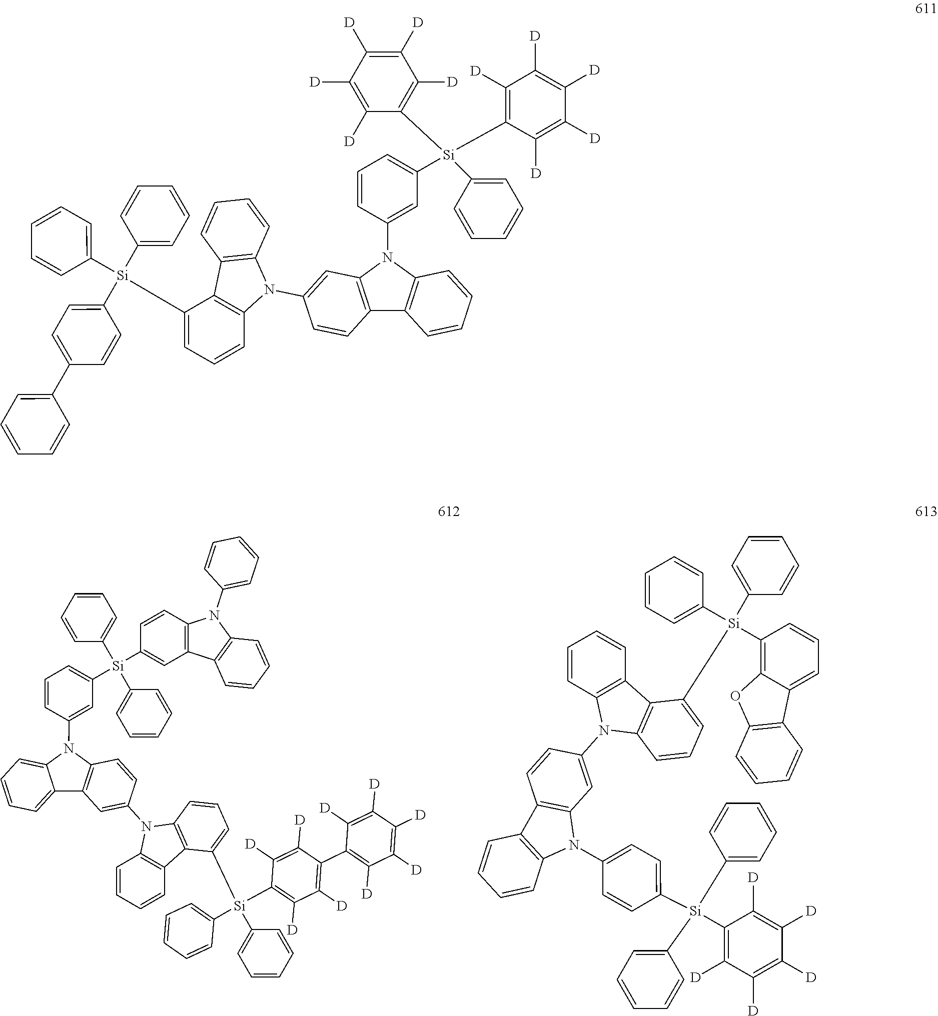

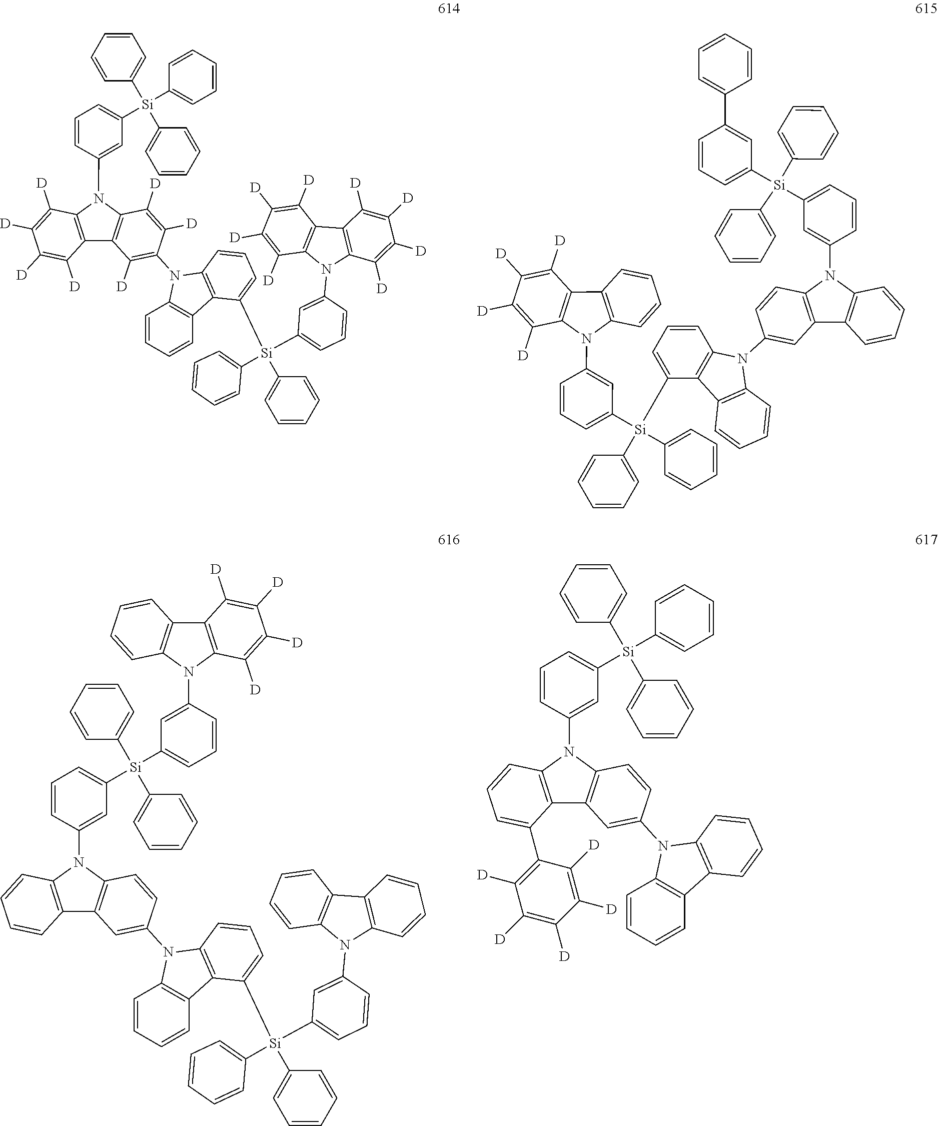

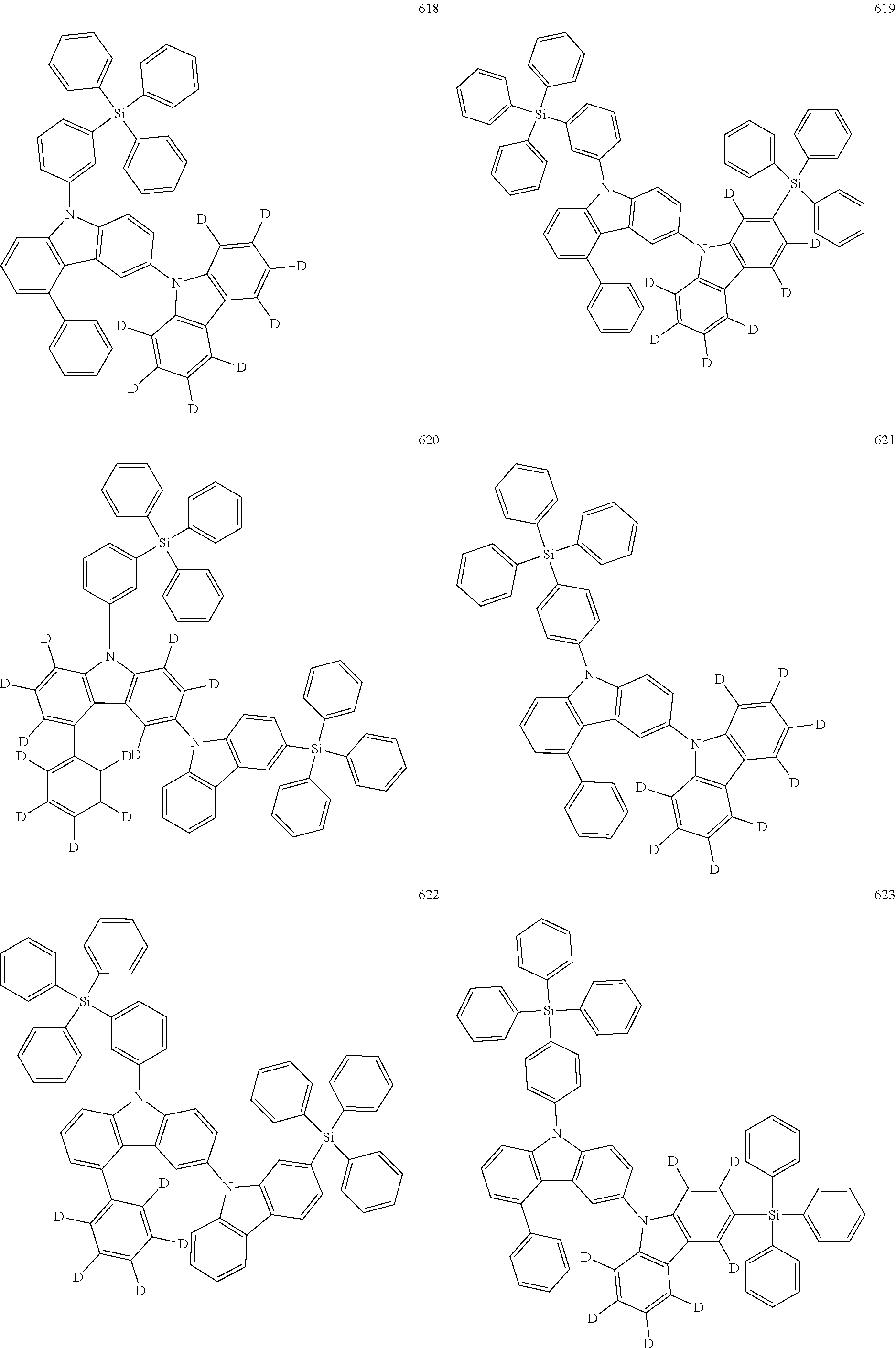

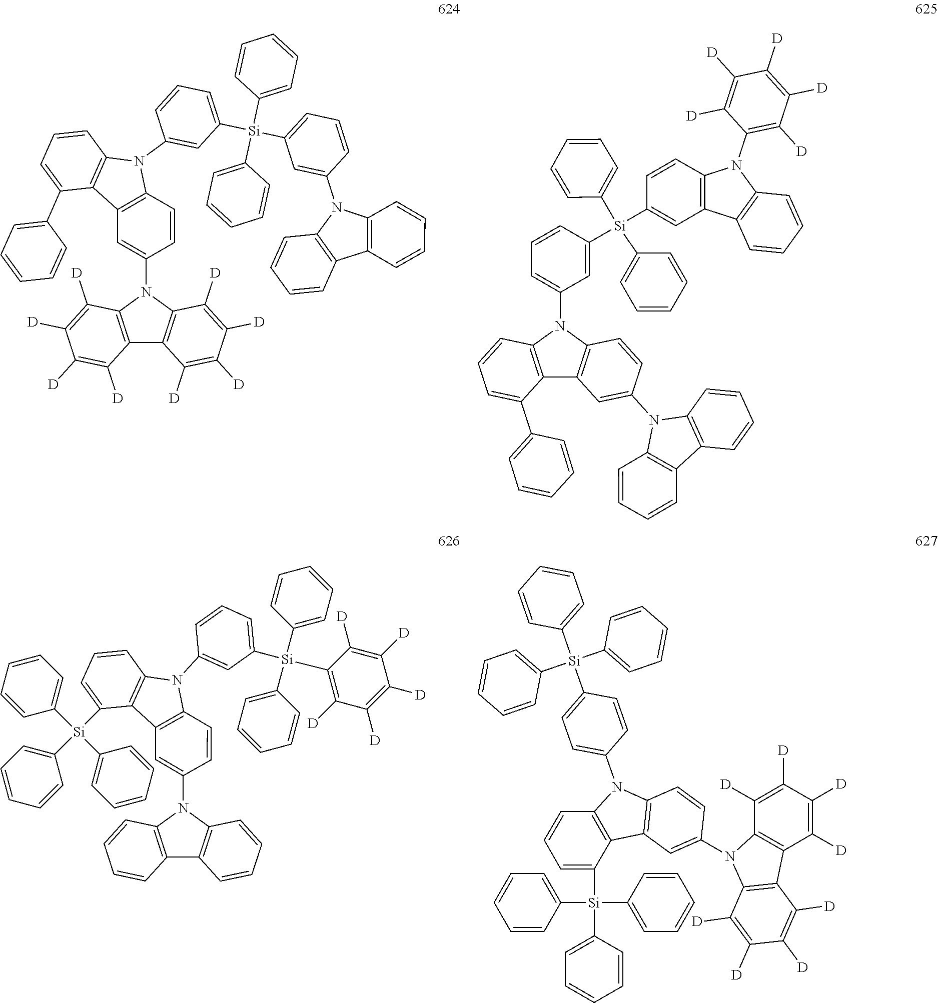

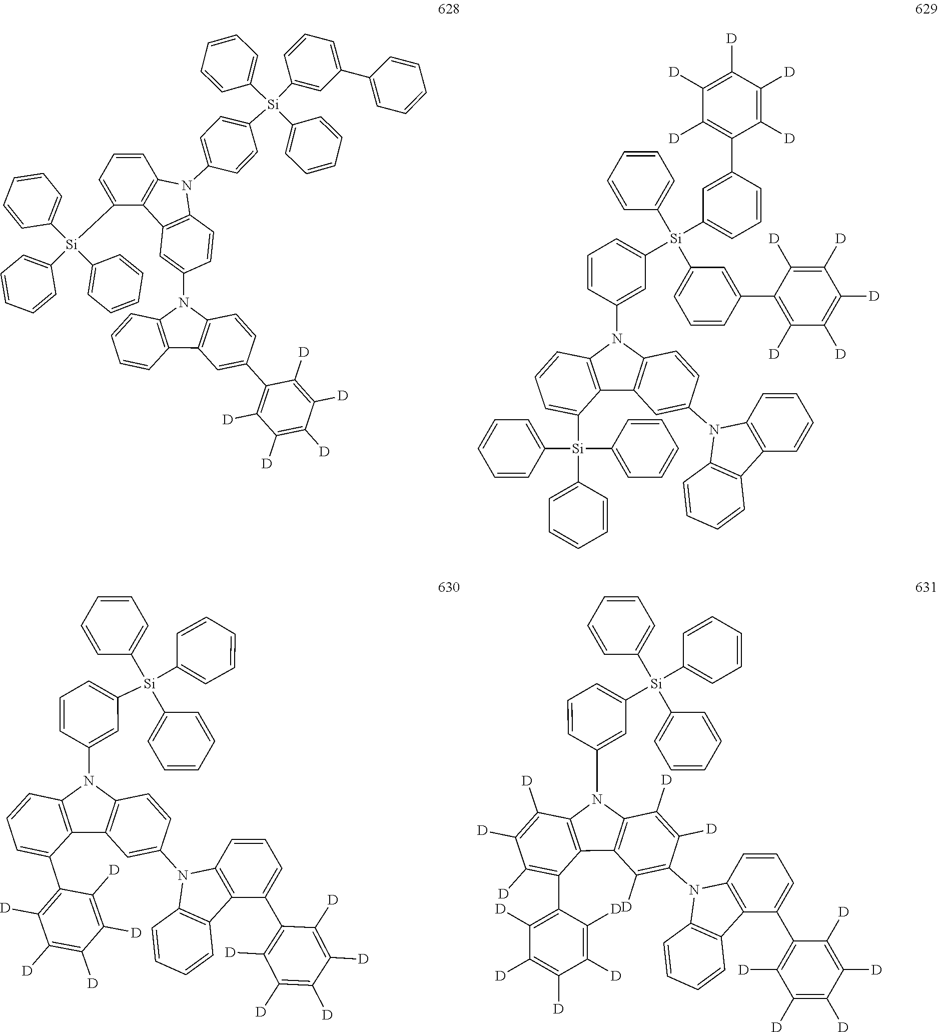

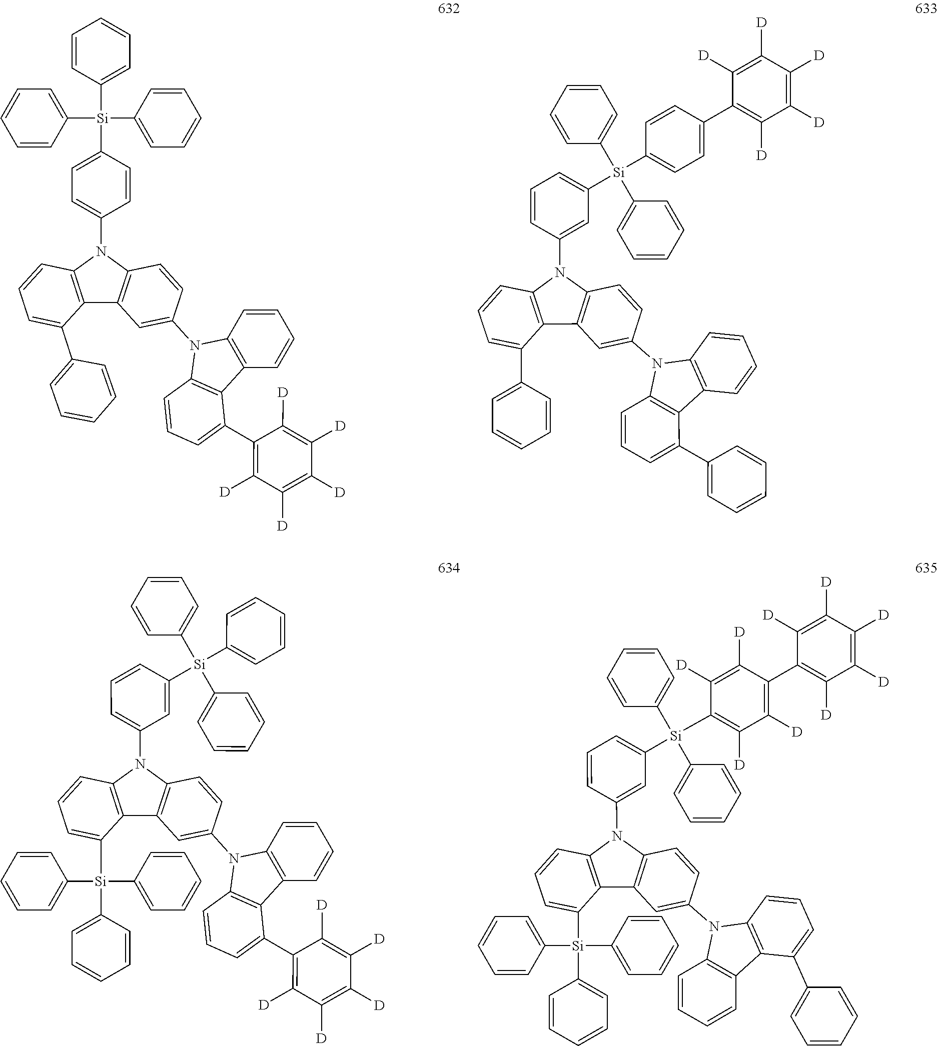

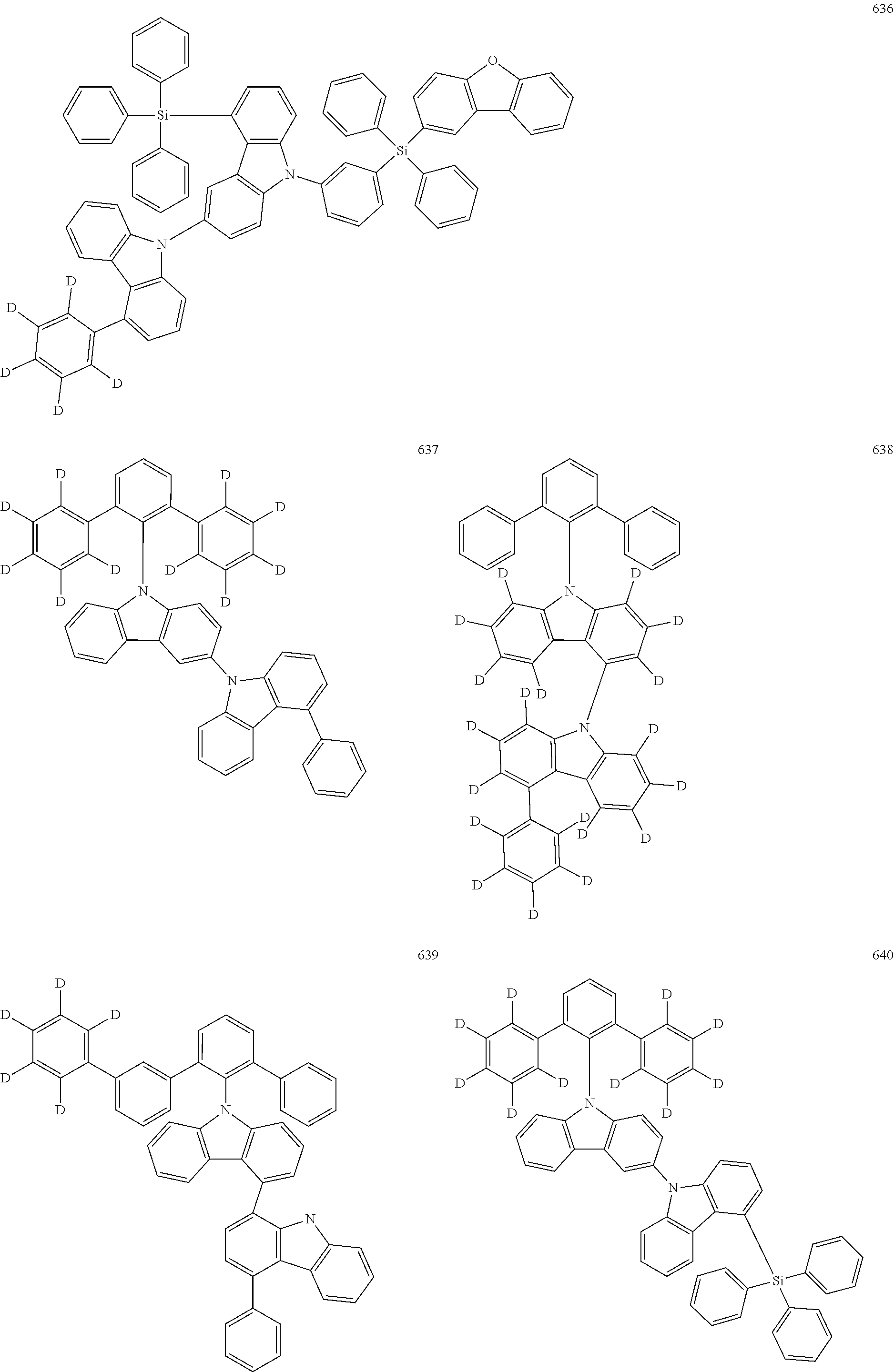

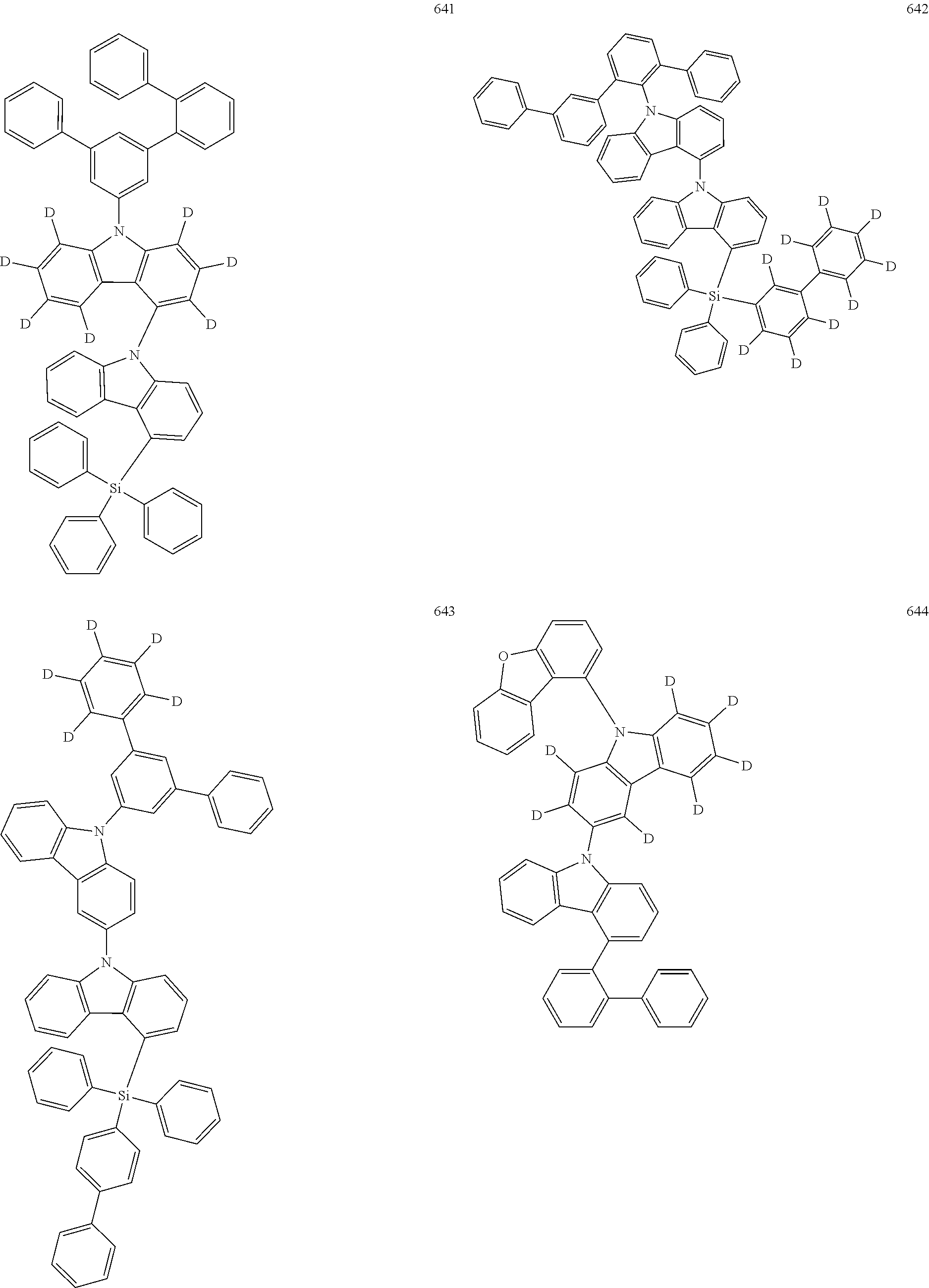

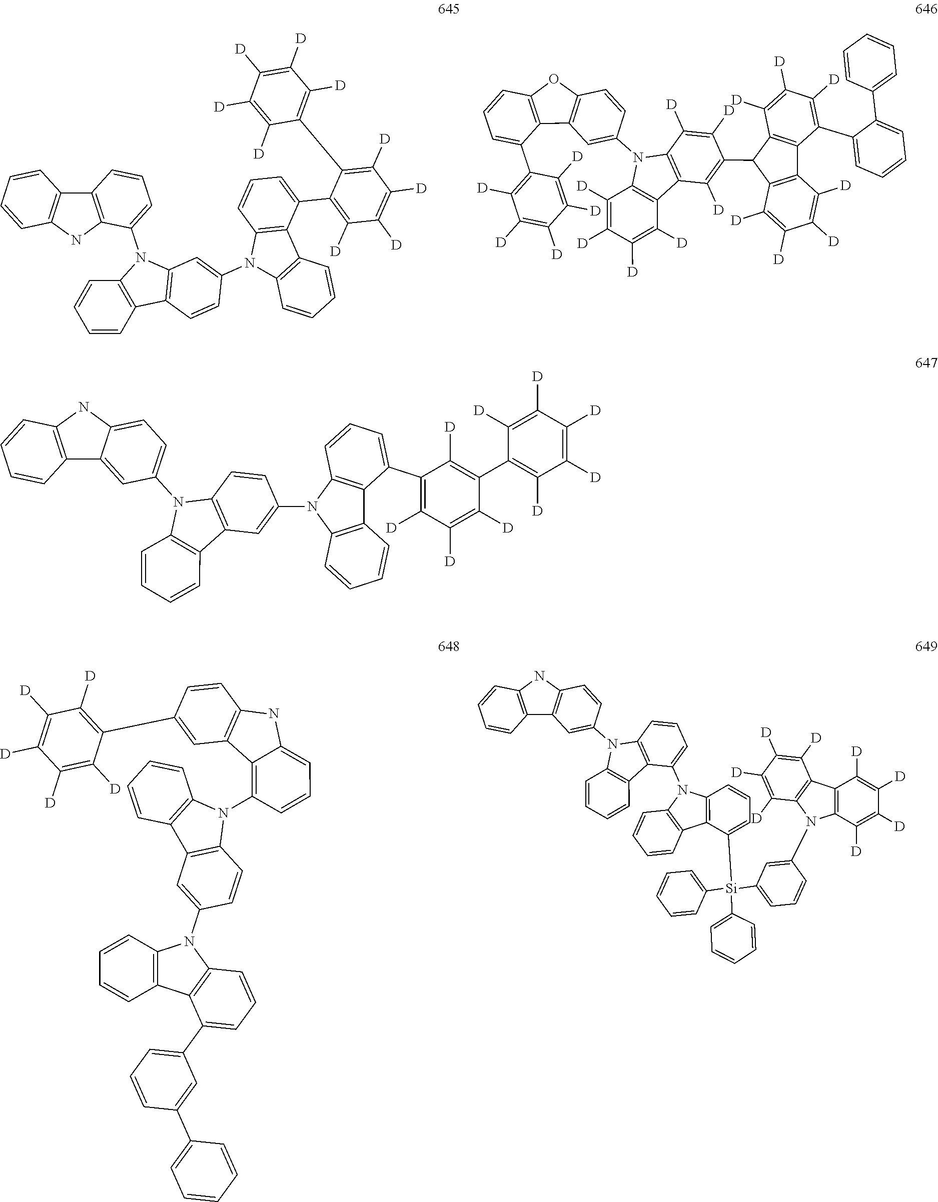

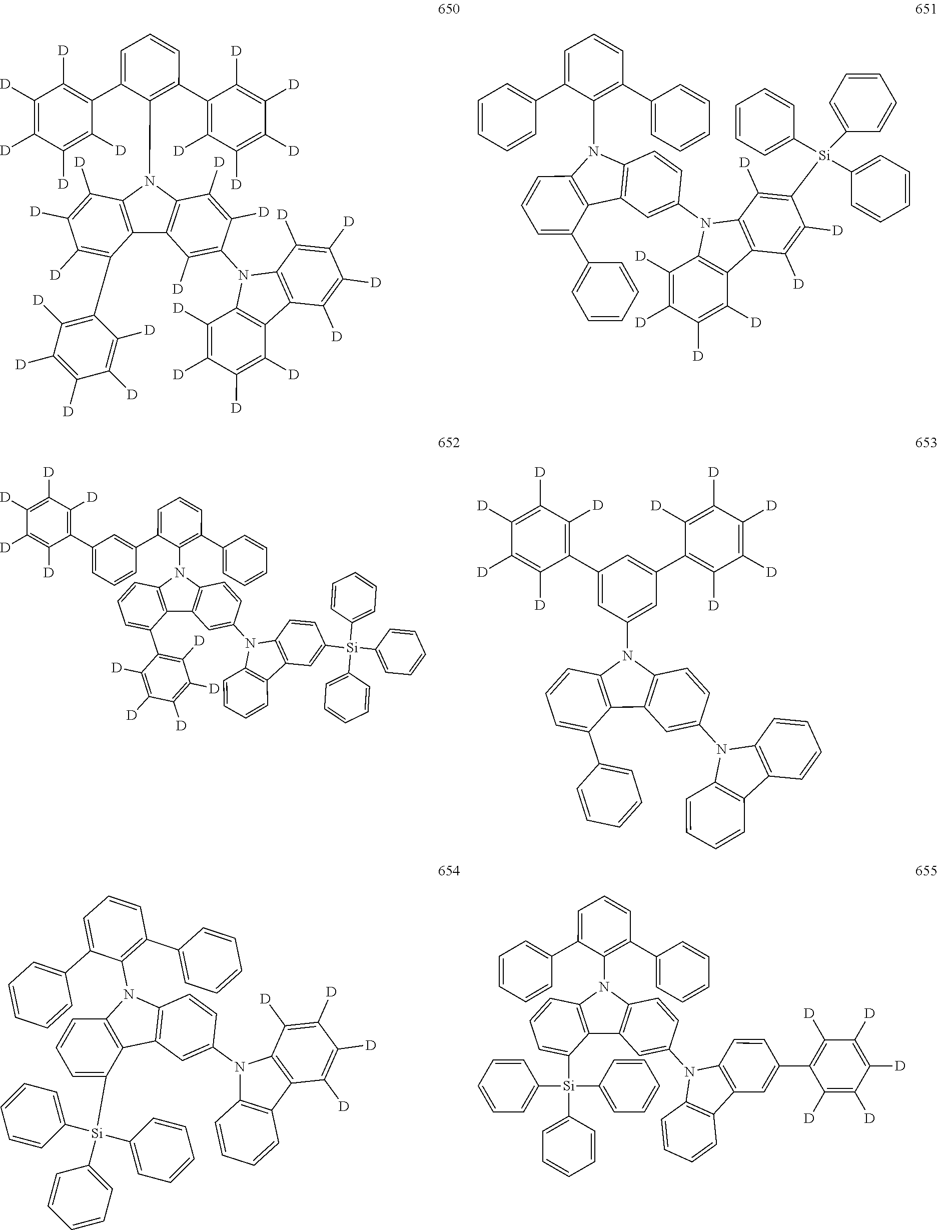

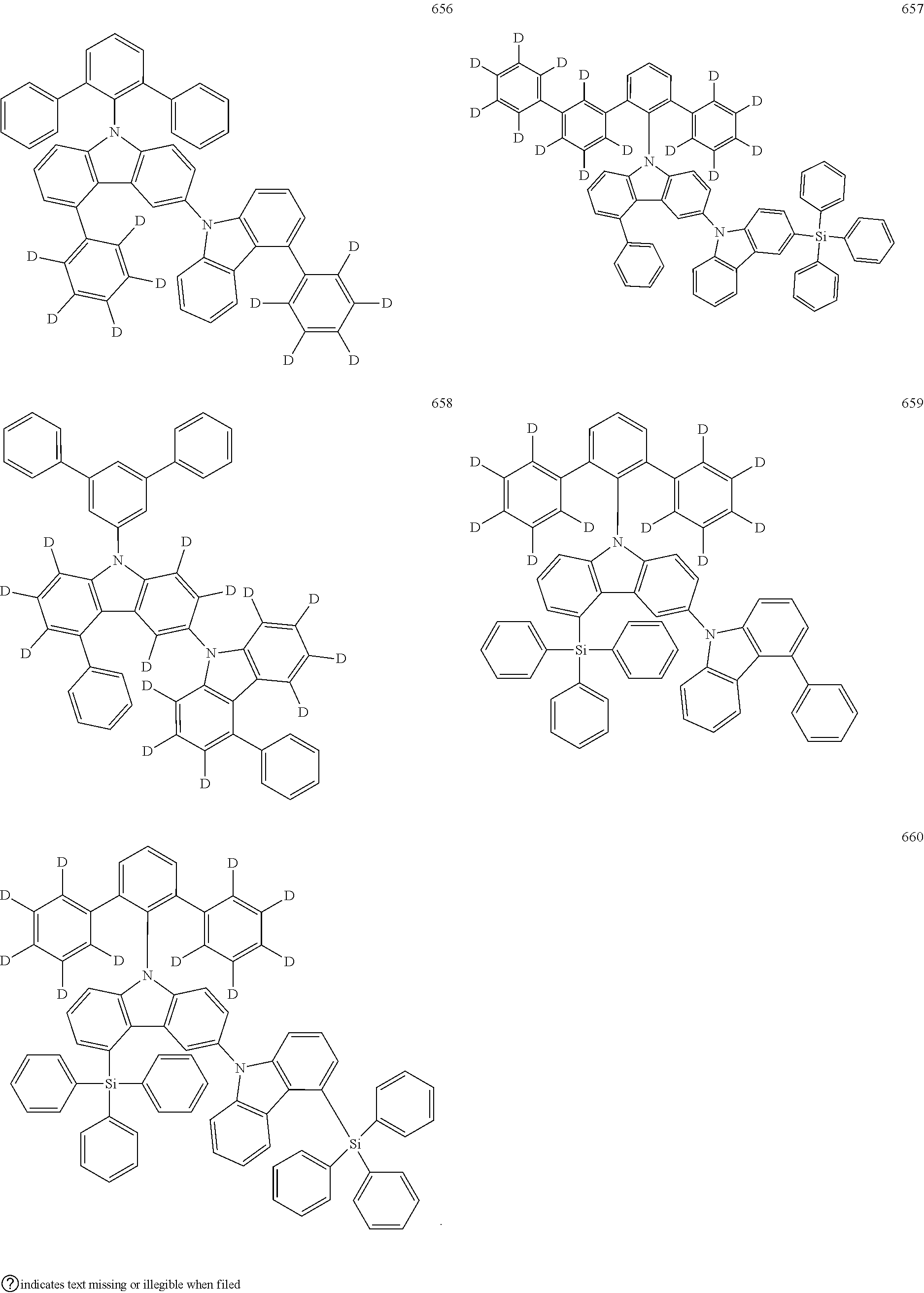

In the particular compounds suggested in Compound Group 1, “D” refers to a deuterium atom.

When the nitrogen-containing compound of one or more embodiments acts as the host of an emission layer, high emission efficiency and blue light with high color purity may be accomplished. In comparable light emitting devices, due to the strong charge interaction between a host and a dopant in an emission layer, a first emission peak due to the light emission of the dopant itself and a second emission peak due to the interaction between the host and the dopant are produced, and color deterioration and efficiency degradation may occur. In addition, in the case of the host, when a difference between the level of the lowest unoccupied molecular orbital (LUMO) and the LUMO energy level of the dopant is large, an energy barrier may be formed between the host and the dopant. The formation of such an energy barrier may accelerate the deterioration of a material and may be a factor (e.g., a main factor) in degrading the device life.

The nitrogen-containing compound of one or more embodiments includes a first carbazole group and a second carbazole group. To the first carbazole group and/or the second carbazole group, a first substituent including a phenyl moiety and/or a triphenyl silane moiety is bonded. Accordingly, the interaction of the nitrogen-containing compound of one or more embodiments with a dopant may be reduced to provide emission color of deep blue light with high or suitable color purity. In some embodiments, the nitrogen-containing compound of one or more embodiments may suppress or reduce the degradation of efficiency and the deterioration of lifetime by an energy barrier with the dopant. For example, the structure and the specific arrangement of the nitrogen-containing compound of one or more embodiments may suppress or reduce energy transfer with a dopant in an emission layer. Accordingly, light emission due to the interaction with the dopant may be suppressed or reduced, and the emission efficiency and device life may be improved even further.

In some embodiments, the nitrogen-containing compound of one or more embodiments introduces the first substituent having a structure of large steric hindrance, and thermal stability of a molecule may be improved, distance between adjacent molecules may increase, and dexter energy transfer may be suppressed or reduced to suppress or reduce lifetime deterioration due to the increase of the concentration of triplet to improve device life even further.

In the nitrogen-containing compound of one or more embodiments, the first substituent is connected with carbon of position 4 of the first carbazole group and/or carbon of position 4 of the second carbazole group. Different from carbon of position 3, the carbon of position 4 of the first carbazole group and/or the second carbazole group does not have a highest occupied molecular orbital (HOMO) energy level of the molecule, and the nitrogen-containing compound of one or more embodiments may have increased triplet energy difference when compared to a case of connecting the substituent with the carbon of position 3, thereby improving emission efficiency. Accordingly, the nitrogen-containing compound of one or more embodiments may show improved color purity and life characteristics, and increase a triplet energy level difference to improve emission efficiency.

In one or more embodiments, the emission layer EML may include a host and a dopant and may include the nitrogen-containing compound as the host. The nitrogen-containing compound of one or more embodiments, represented by Formula 1 may be the host material of the emission layer.

For example, in the light emitting device ED of one or more embodiments, the emission layer EML may include a host for emitting phosphorescence (e.g., a host for phosphorescent emission) and a dopant for emitting phosphorescence (e.g., a dopant capable of phosphorescent emission), and may include the nitrogen-containing compound of one or more embodiments as the host for emitting phosphorescence. In some embodiments, in the light emitting device ED of one or more embodiments, the emission layer EML may include a host for emitting fluorescence (e.g., a host for fluorescent emission) and a dopant for emitting fluorescence (e.g., a dopant capable of fluorescent emission), and the nitrogen-containing compound of one or more embodiments may include the host for emitting fluorescence.

In the light emitting device ED of one or more embodiments, an emission layer EML may include a host for emitting delayed fluorescence (e.g., a host for delayed fluorescent emission) and a dopant for emitting delayed fluorescence (e.g., a dopant capable of delayed fluorescent emission), and may include the nitrogen-containing compound of one or more embodiments as the host for emitting delayed fluorescence. In the light emitting device ED of one or more embodiments, an emission layer EML may include a host for emitting blue thermally activated delayed fluorescence (TADF) (e.g., a host for blue thermally activated delayed fluorescent emission) and a dopant for emitting blue thermally activated delayed fluorescence (e.g., a dopant capable of blue thermally activated delayed fluorescent emission), and may include the nitrogen-containing compound of one or more embodiments as the host for emitting blue thermally activated delayed fluorescence. The emission layer EML may include at least one selected from among the nitrogen-containing compounds represented in Compound Group 1, as the host material of the emission layer EML.

In the light emitting device ED of one or more embodiments, a host may not emit light in the light emitting device ED but may play the role of transferring energy to a dopant. The emission layer EML may include one or more hosts. For example, the emission layer EML may include two types (kinds) of different hosts. However, one or more embodiments of the present disclosure is not limited thereto, and the emission layer EML may include one type or kind of a host, or a mixture of two or more types (kinds) of different hosts.

In one or more embodiments, the emission layer EML may include one or more suitable different hosts. For example, the host may include a first compound represented by Formula 1, and a second compound which is different from the first compound. In one or more embodiments, the host may include the first compound represented by Formula 1, and the second compound represented by Formula ET-1 below. The first compound may be a hole transport host, and the second compound may be an electron transport host. In one or more embodiments, the emission layer EML includes the first compound and the second compound, and the first compound and the second compound may form exciplex.

In the light emitting device ED of one or more embodiments, exciplexes may be formed by a hole transport host and an electron transport host in an emission layer. In this case, the triplet energy of the exciplexes formed by the hole transport host and the electron transport host may correspond to the difference between the energy level of the lowest unoccupied molecular orbital (LUMO) of the electron transport host and the energy level the highest occupied molecular orbital (HOMO) of the hole transport host.

For example, the absolute value of the triplet energy level (T1) of the exciplexes formed by the hole transport host and the electron transport host may be about 2.4 eV to about 3.0 eV. In some embodiments, the triplet energy of the exciplexes may be a value smaller than the energy gap of each host material. The exciplexes may have a triplet energy of about 3.0 eV or less, which is (or is smaller than) the energy gap of the hole transport host and/or is (or is smaller than) the electron transport host.

In one or more embodiments, the emission layer EML may include a hole transport host represented by Formula HT-1 together with the first compound.

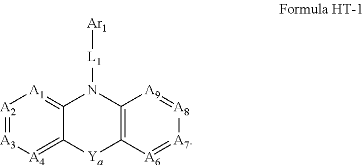

In Formula HT-1, A₁ to A₉ may each independently be N or CR₄₁. For example, all of A₁ to A₉ may be CR₄₁. In some embodiments, any one selected from among A₁ to A₉ may be N, and the remainder may be CR₄₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, and/or the like, but one or more embodiments of the present disclosure is not limited thereto.

In Formula HT-1, Yₐ may be a direct linkage, CR₄₂R₄₃, or SiR₄₄R₄₅. For example, it may mean that two benzene rings connected with the nitrogen atom of Formula HT-1 may be connected via a direct linkage,

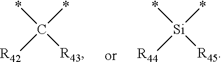

In Formula HT-1, when Yₐ is the direct linkage, the compound represented by Formula HT-1 may include a carbazole moiety.

In Formula HT-1, Ar₁ may be a substituted or unsubstituted aryl group of 6 to 30 ring-forming carbon atoms, or a substituted or unsubstituted heteroaryl group of 2 to 30 ring-forming carbon atoms. For example, Ar₁ may be a substituted or unsubstituted carbazole group, a substituted or unsubstituted dibenzofuran group, a substituted or unsubstituted dibenzothiophene group, a substituted or unsubstituted biphenyl group, and/or the like, but one or more embodiments of the present disclosure is not limited thereto.

In Formula HT-1, R₄₁ to R₄₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 60 ring-forming carbon atoms. In some embodiments, R₄₁ to R₄₅ may each independently be combined with an adjacent group to form a ring. For example, R₄₁ to R₄₅ may each independently be a hydrogen atom or a deuterium atom. R₄₁ to R₄₅ may each independently be an unsubstituted methyl group or an unsubstituted phenyl group.

In one or more embodiments, the hole transport host represented by Formula HT-1 may be represented by any one selected from among the compounds represented in Compound Group HT. An emission layer EML may include at least one selected from among the compounds represented in Compound Group HT as a hole transport host material.

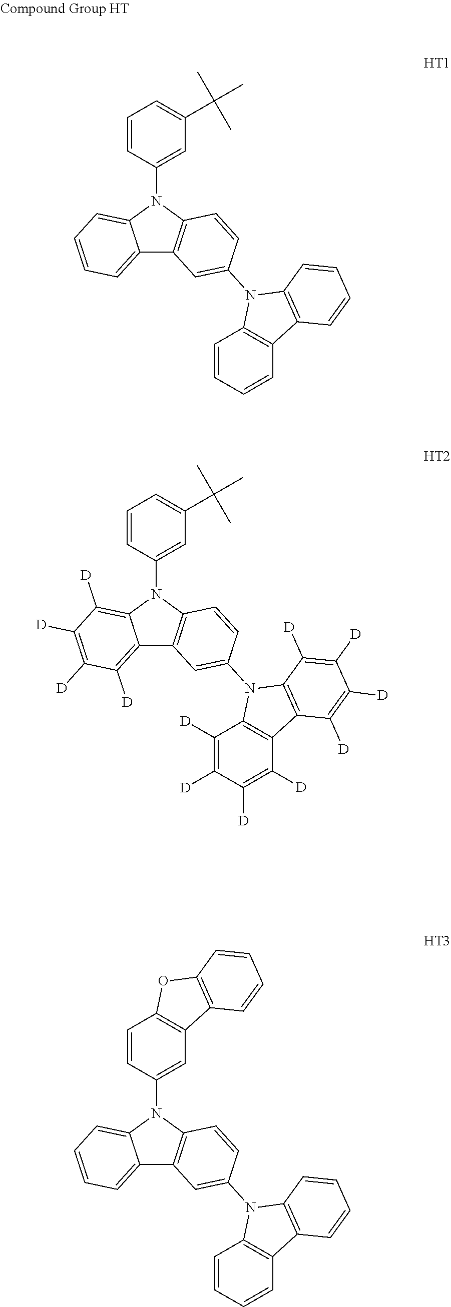 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

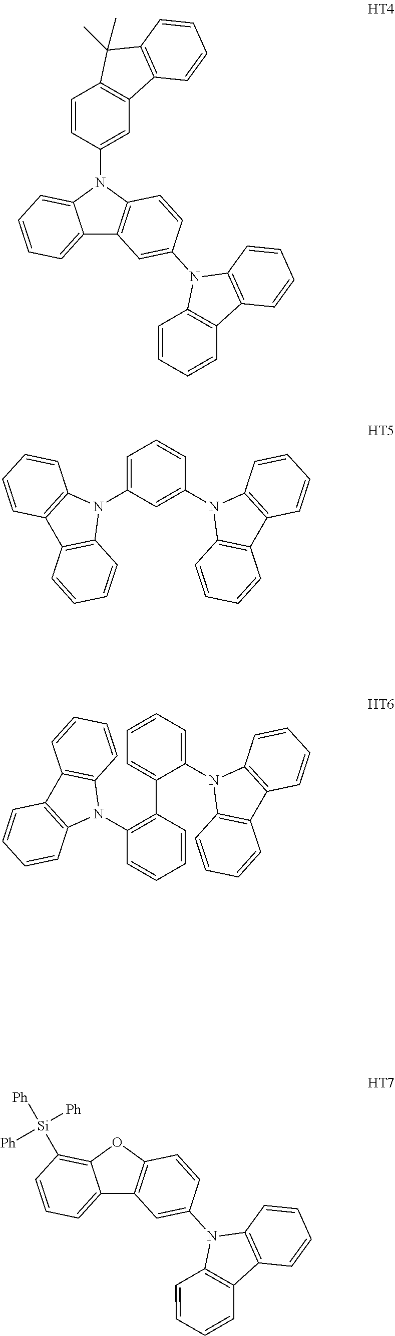 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

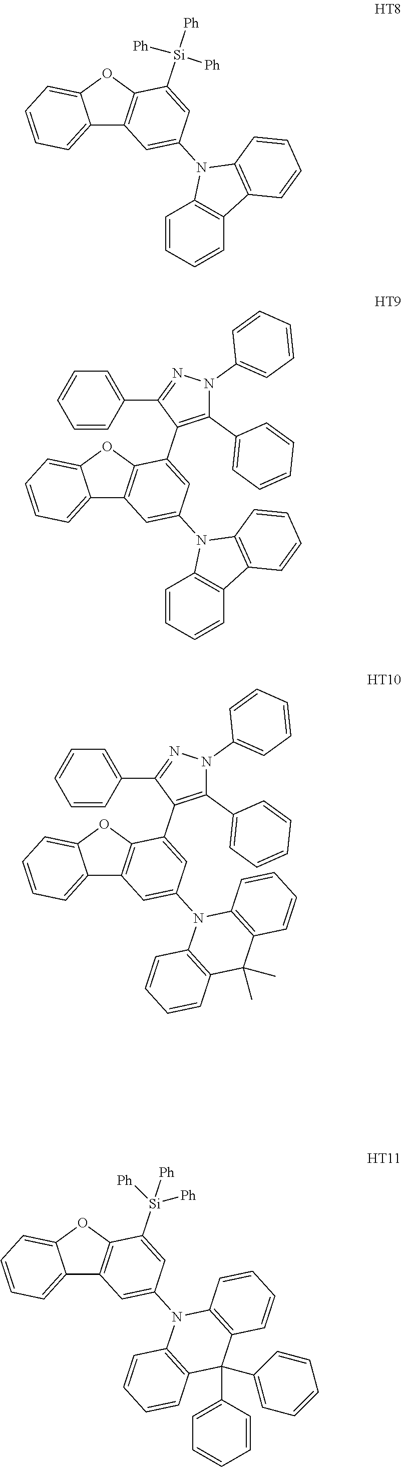 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

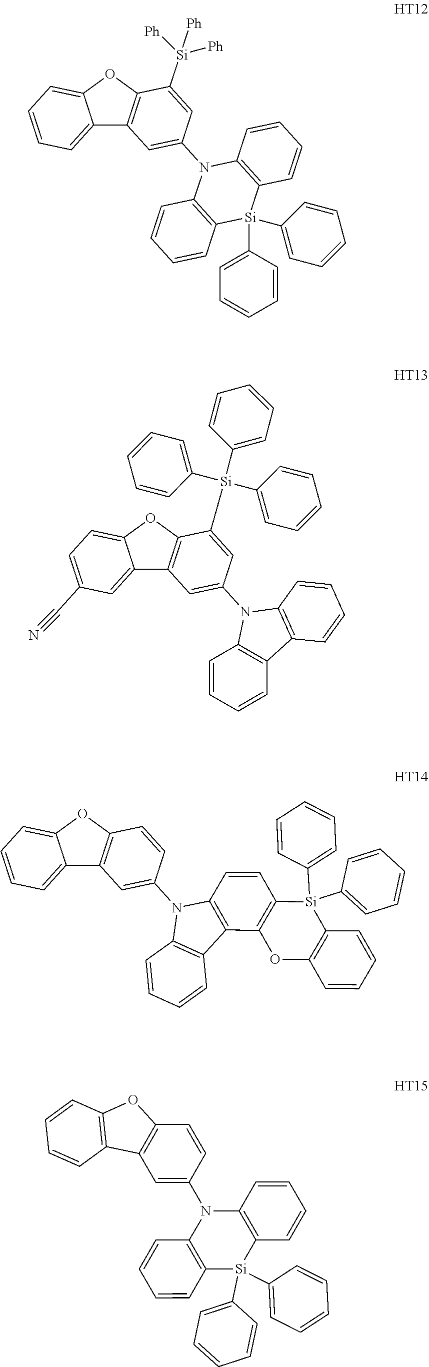 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1]

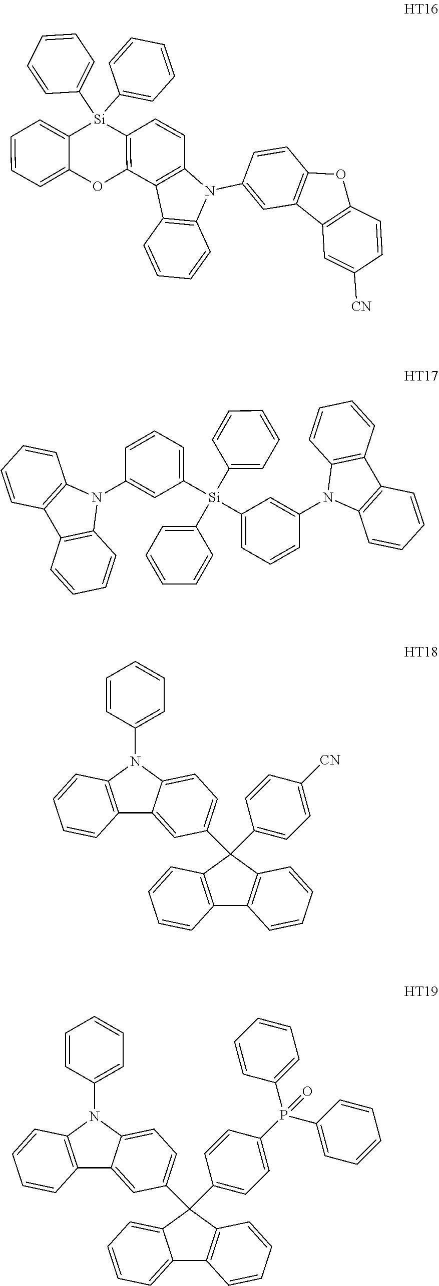 [SMILES: N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1]

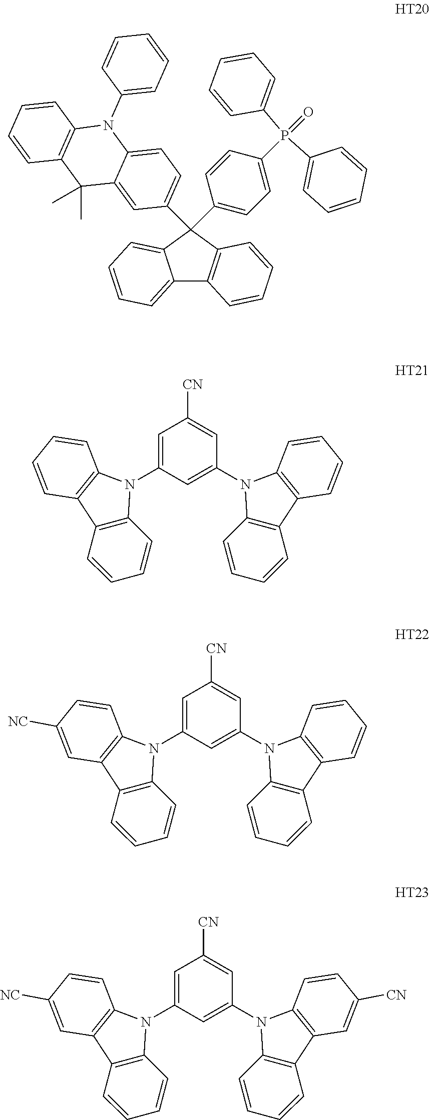 [SMILES: CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1]

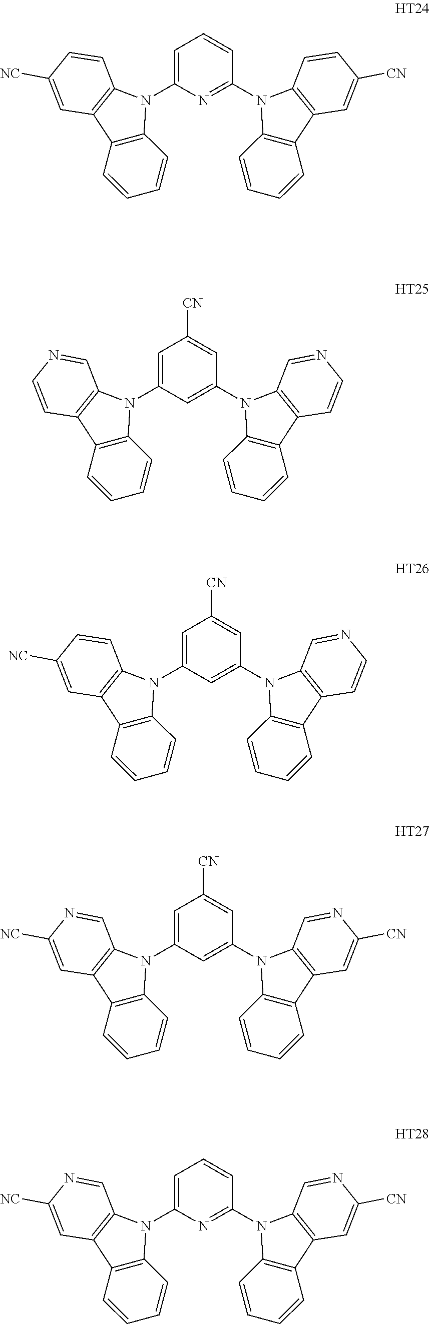 [SMILES: N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

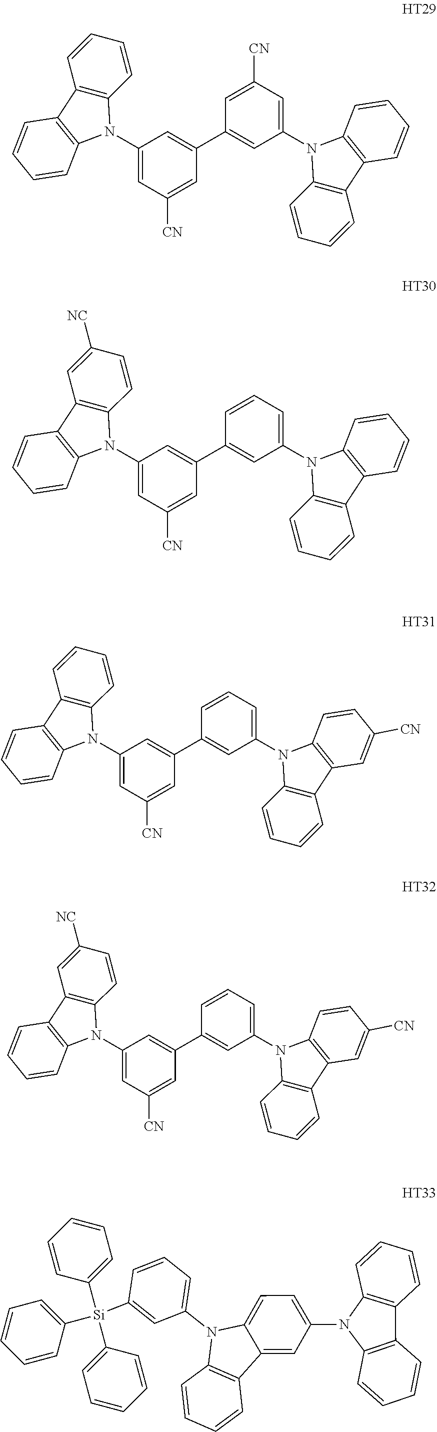 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1 ; c1ccc([Si](c2ccccc2)(c2ccccc2)c2cccc(-n3c4ccccc4c4cc(-n5c6ccccc6c6ccccc65)ccc43)c2)cc1]

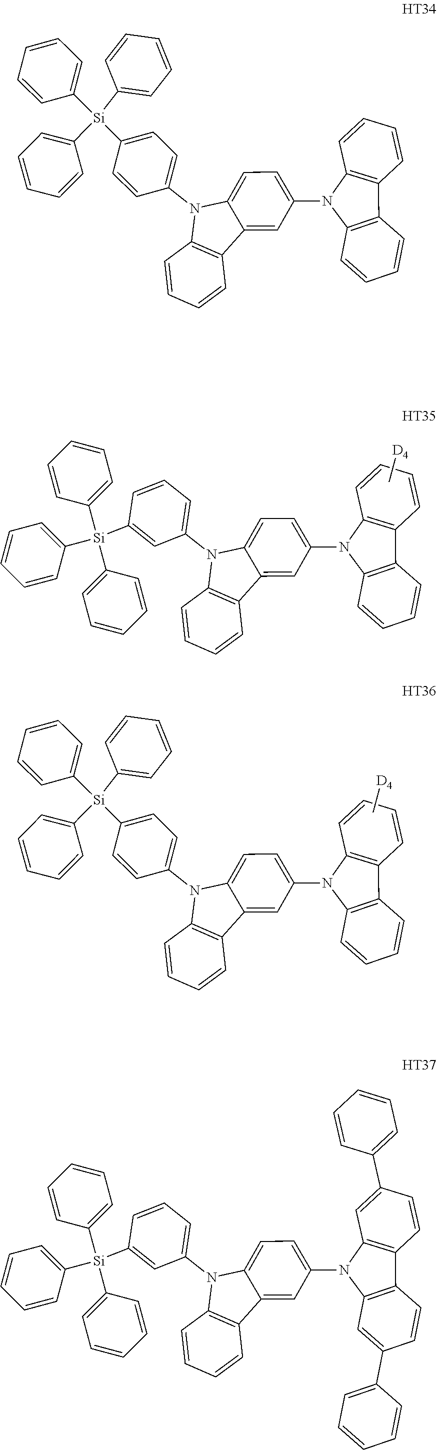

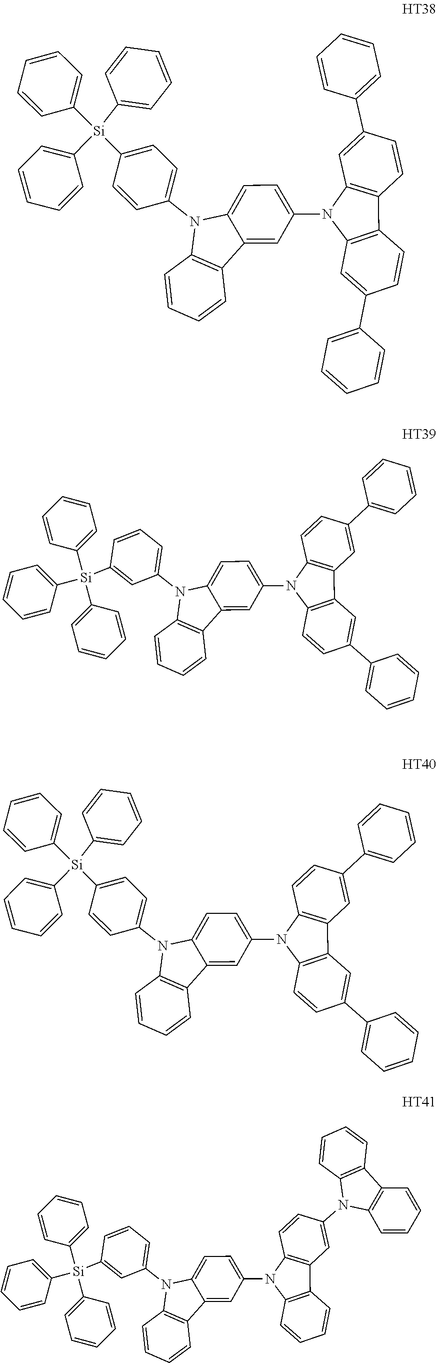 [SMILES: c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1]

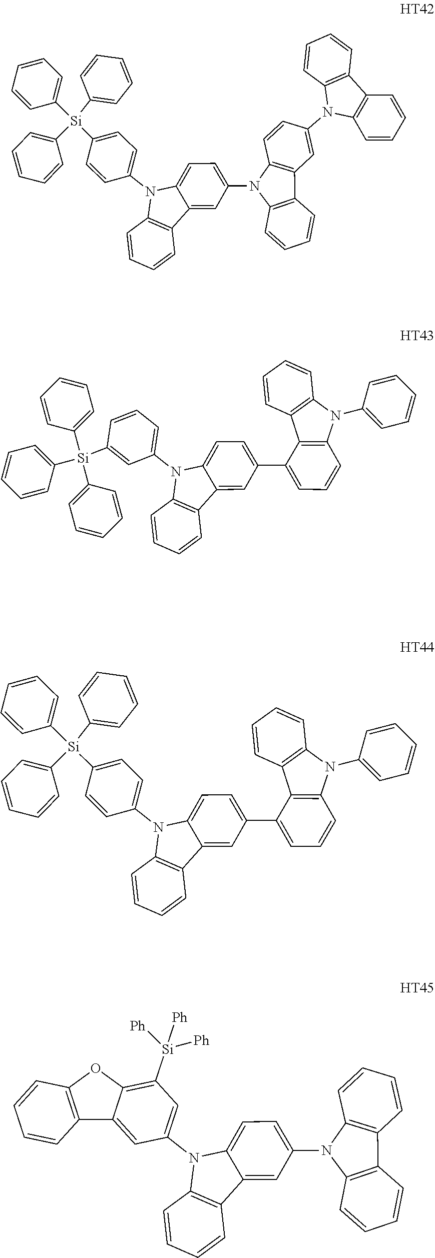 [SMILES: c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1 ; c1ccc(-n2c3ccccc3c3c(-c4ccc5c(c4)c4ccccc4n5-c4ccc([Si](c5ccccc5)(c5ccccc5)c5ccccc5)cc4)cccc32)cc1 ; c1ccc([Si](c2ccccc2)(c2ccccc2)c2cc(-n3c4ccccc4c4cc(-n5c6ccccc6c6ccccc65)ccc43)cc3c2oc2ccccc23)cc1]

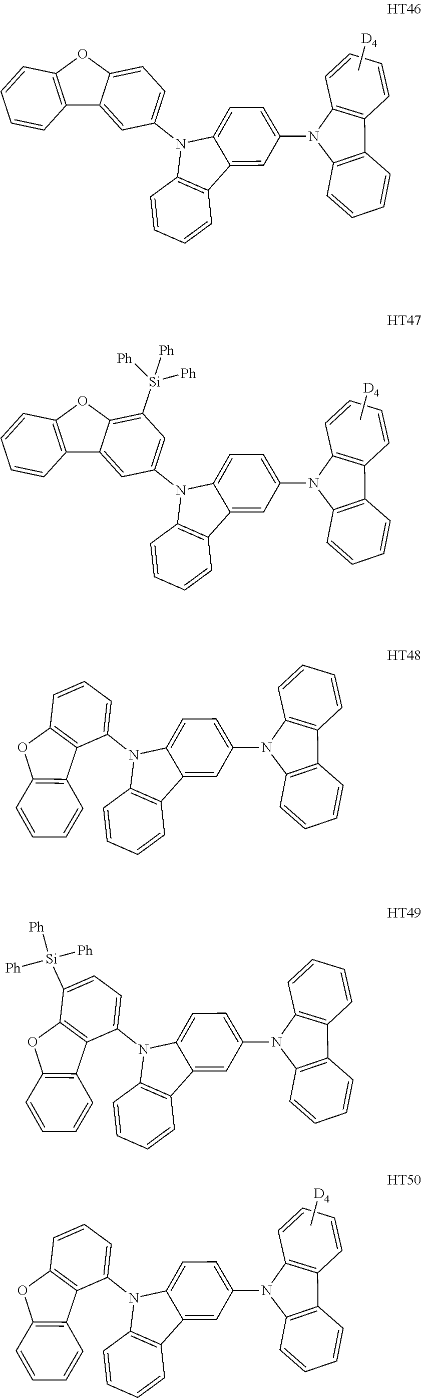

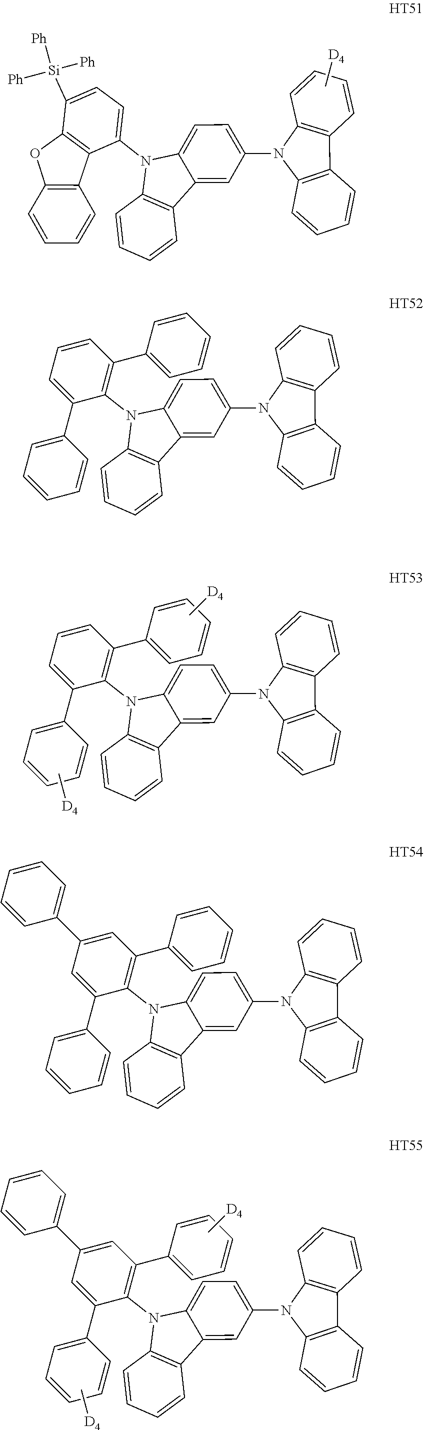

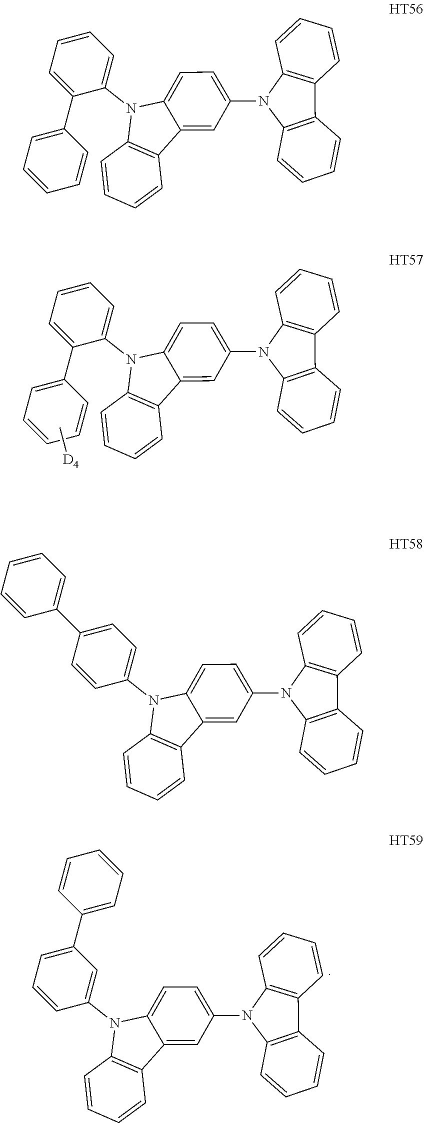

In the compounds in Compound Group HT, “D” refers to a deuterium atom, and “Ph” may refer to an unsubstituted phenyl group.

In one or more embodiments, the emission layer EML may include a second compound represented by Formula ET-1. For example, the second compound may be utilized as an electron transport host material in the emission layer EML.

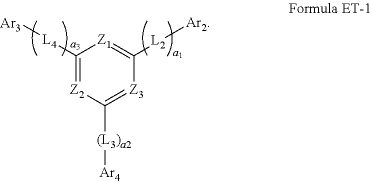

In Formula ET-1, at least one selected from among Z₁ to Z₃ may be N, and the remainder may be CRₐ₃, and Rₐ₃ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

a₁ to a₃ may each independently be an integer of 0 to 10.

L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when each of a1 to as is an integer of 2 or more, L₂ to L₄ may each independently be a substituted or unsubstituted arylene group of 6 to 30 ring-forming carbon atoms, or a substituted or unsubstituted heteroarylene group of 2 to 30 ring-forming carbon atoms.

Ar₂ to Ar₄ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₂ to Ar₄ may be substituted or unsubstituted phenyl groups or substituted or unsubstituted carbazole group.

The second compound may be represented by any one selected from among the compounds in Compound Group 2. The light emitting element ED of one or more embodiments may include any one selected from among the compounds in Compound Group 2.

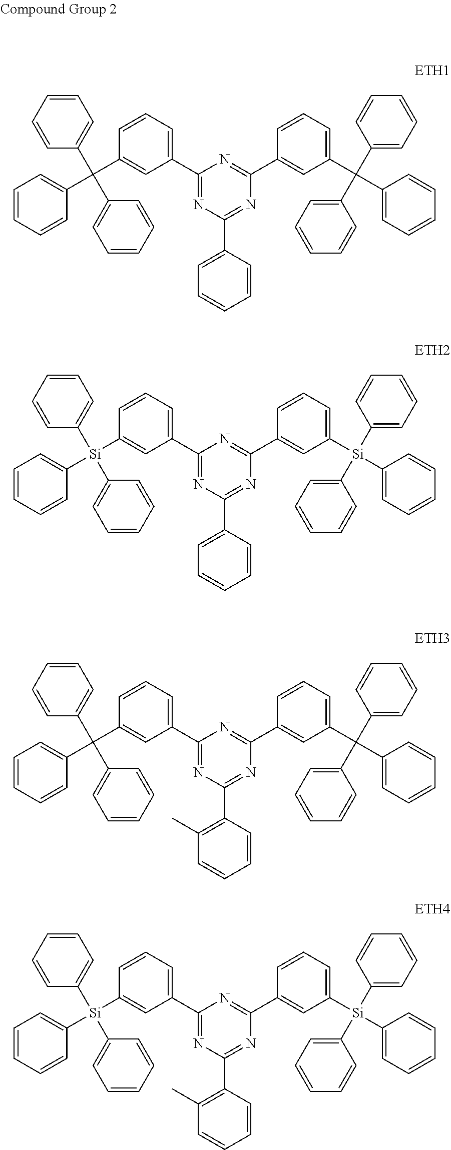 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

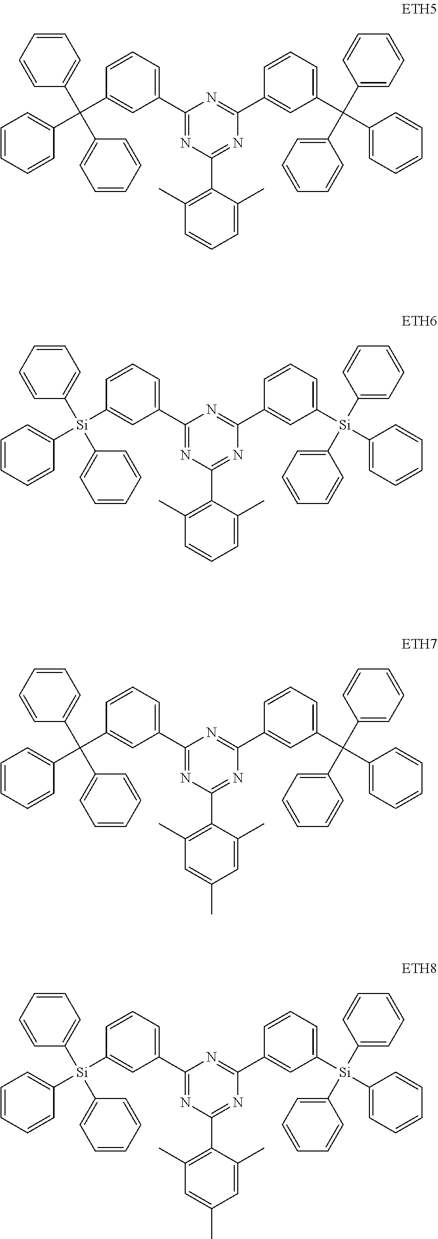 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

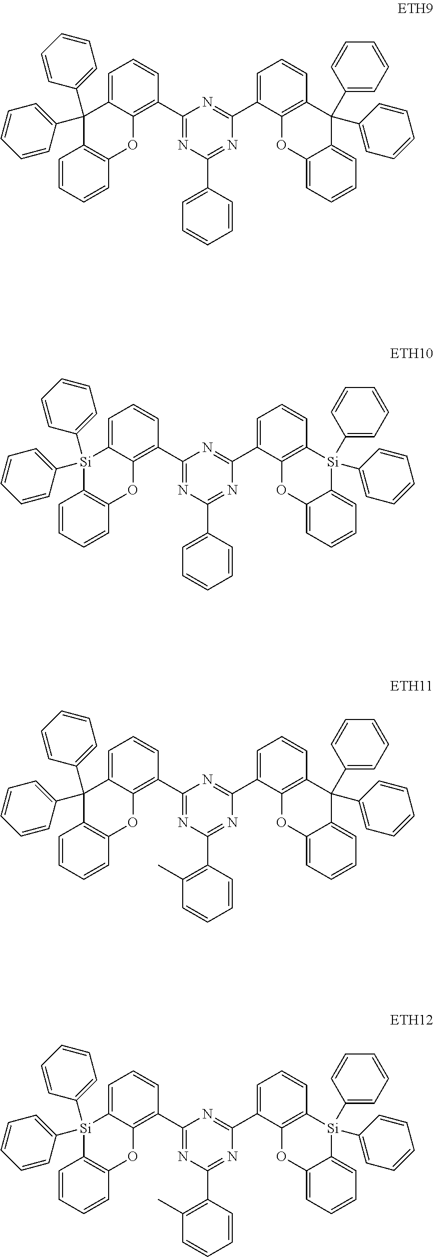 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

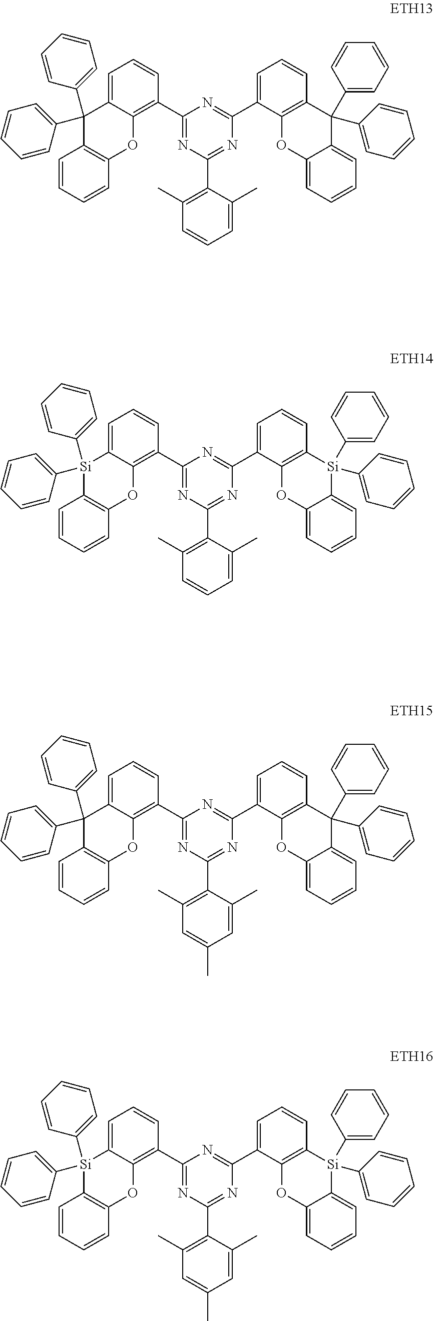 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

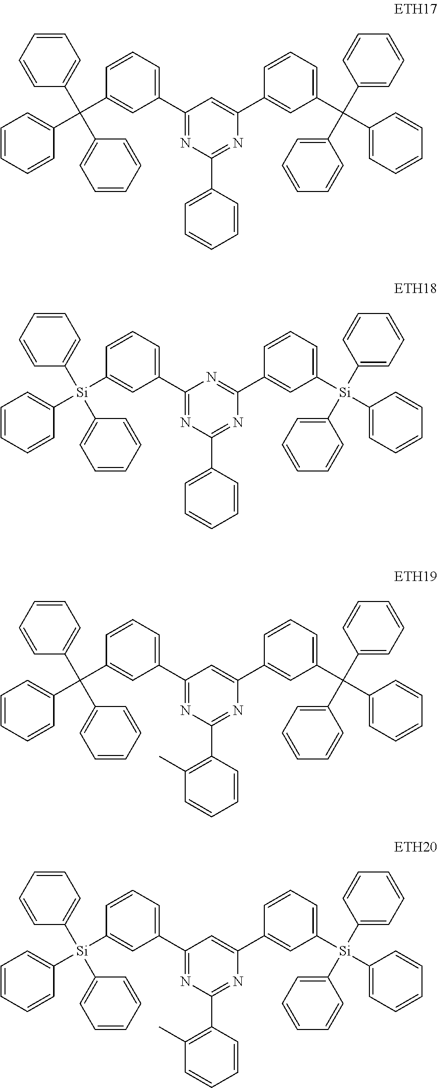 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

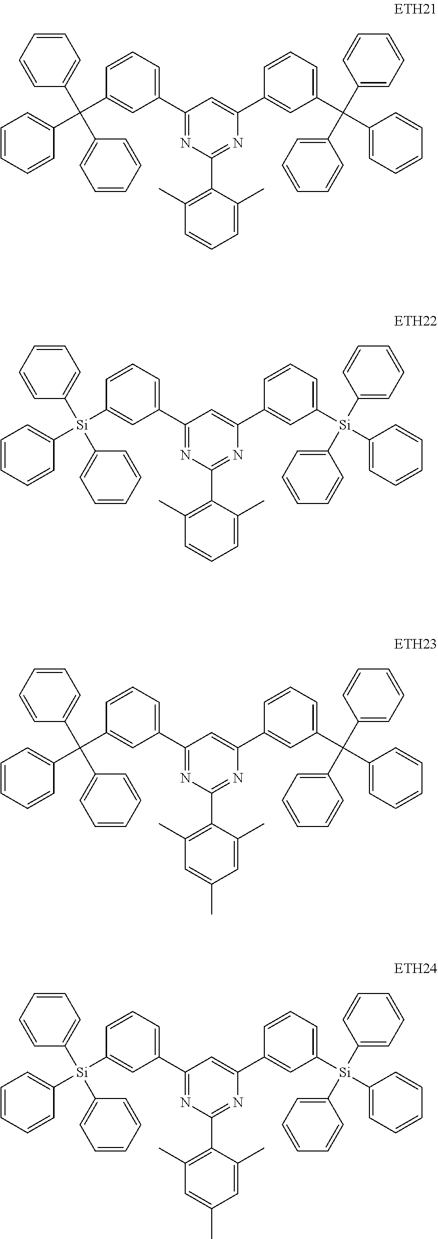 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

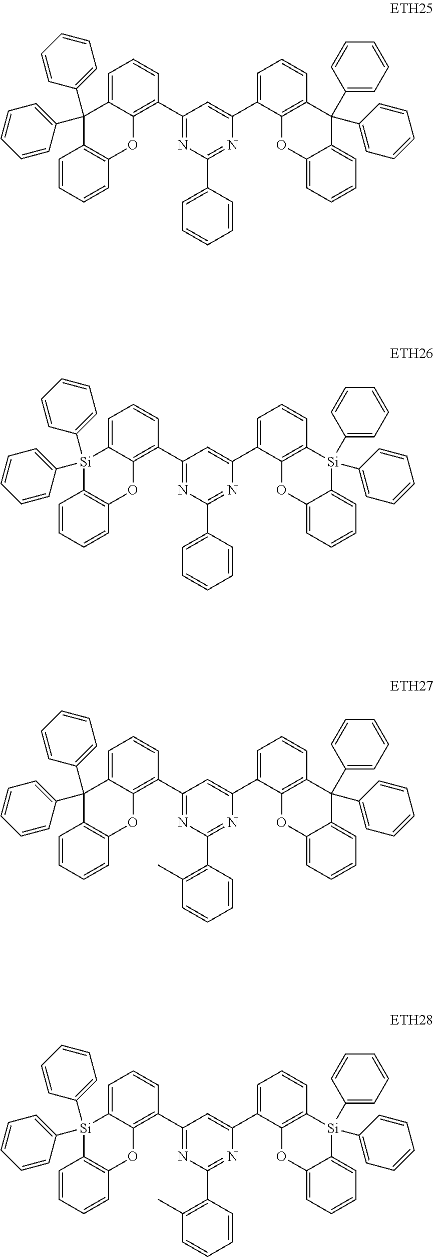 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

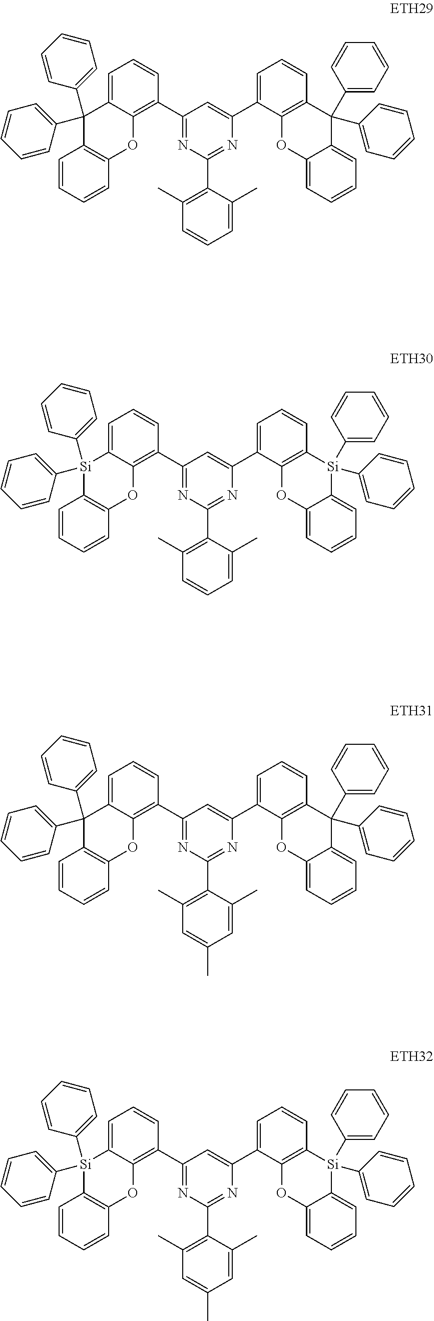 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

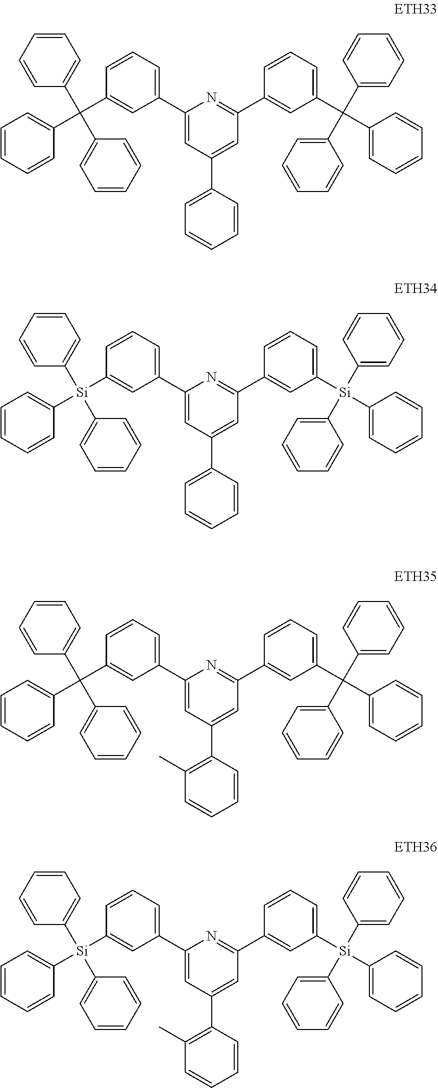 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

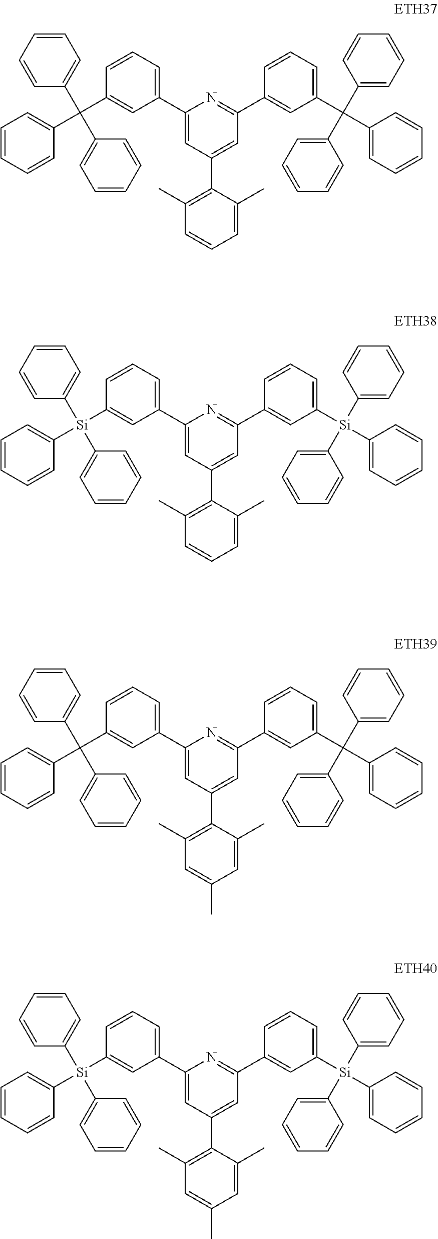 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

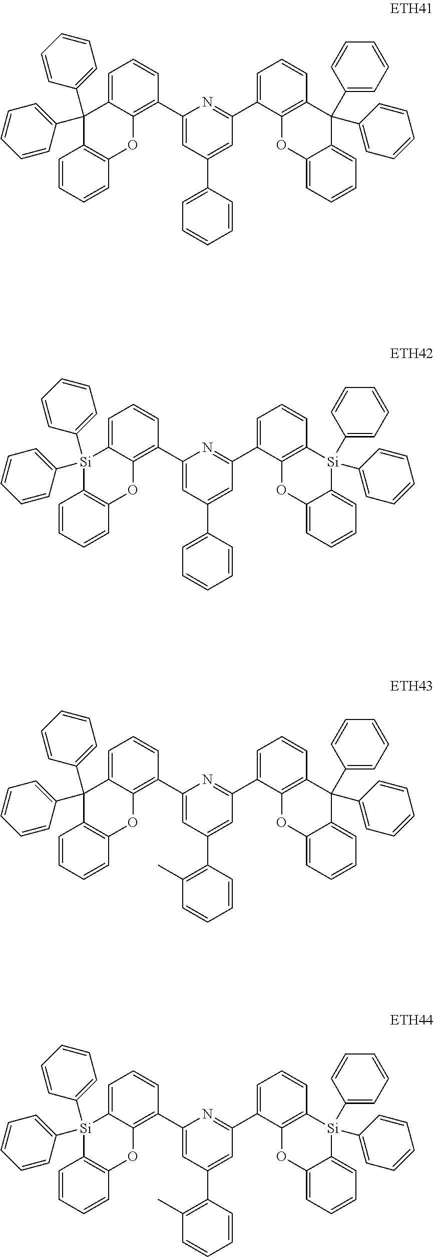 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

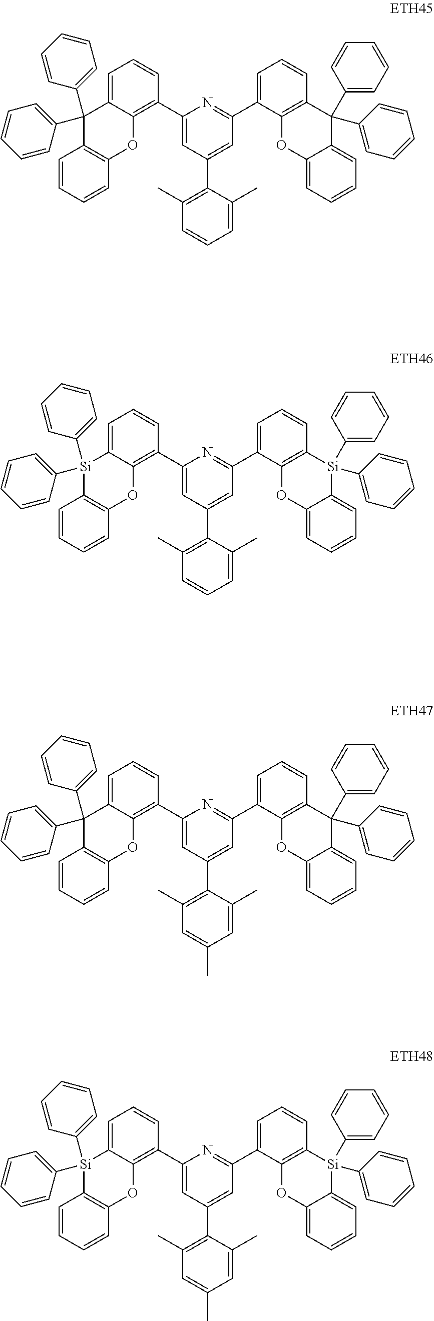 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

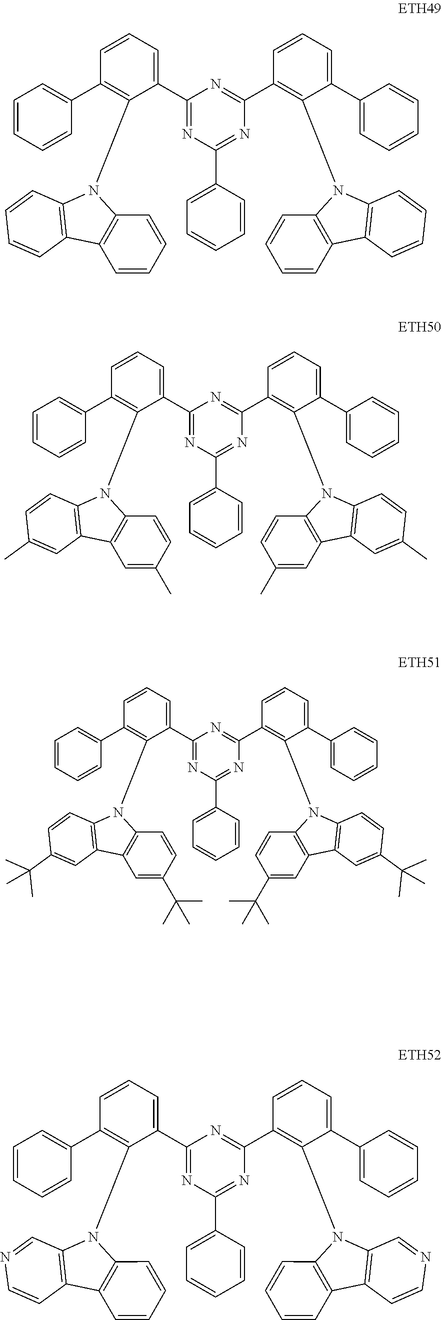

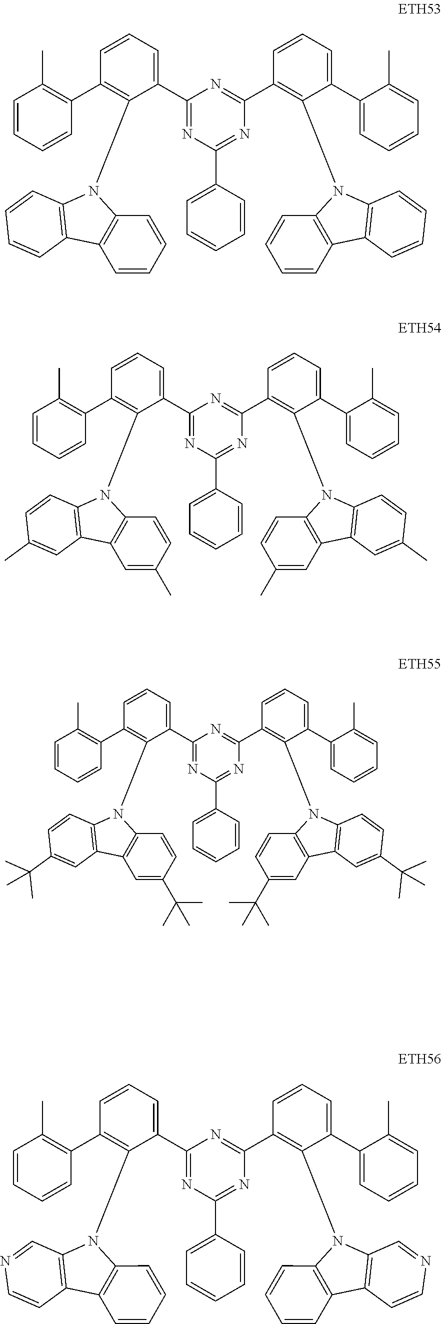

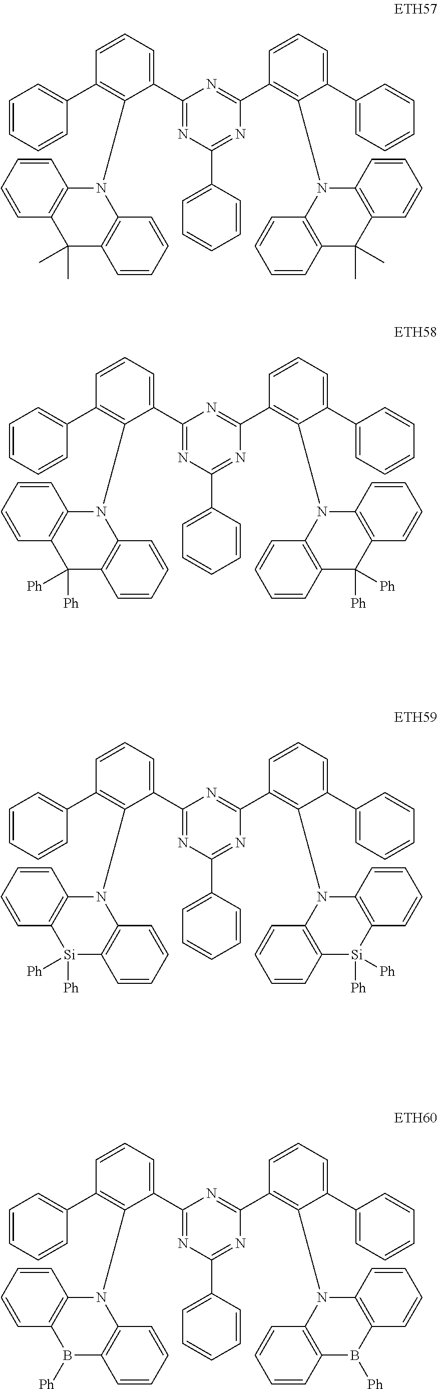

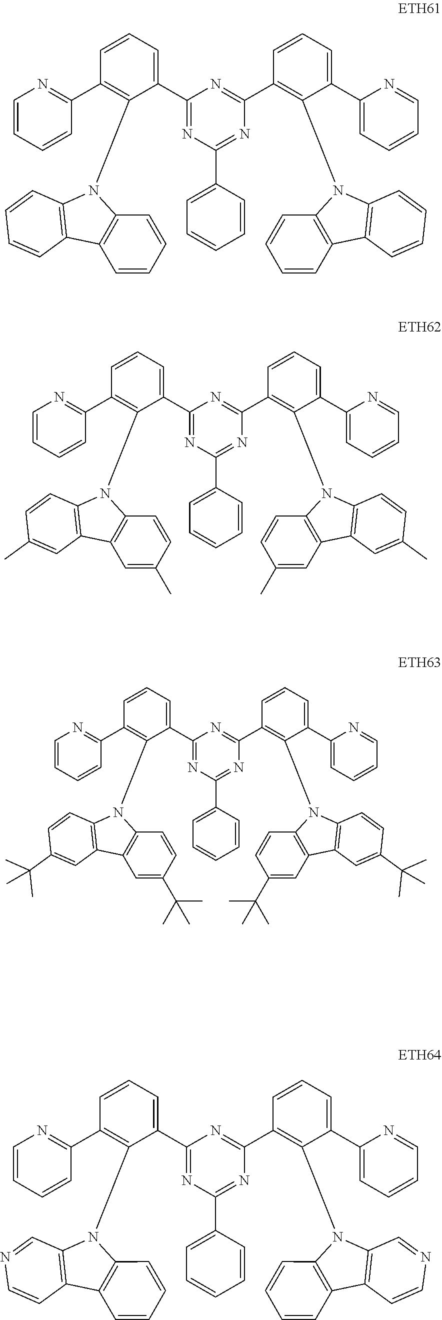

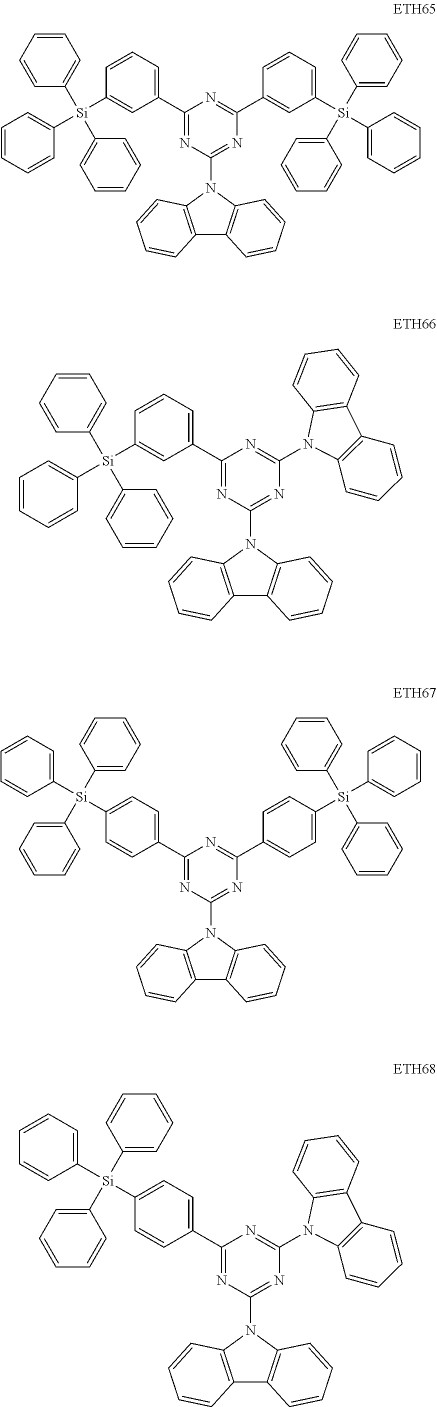 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

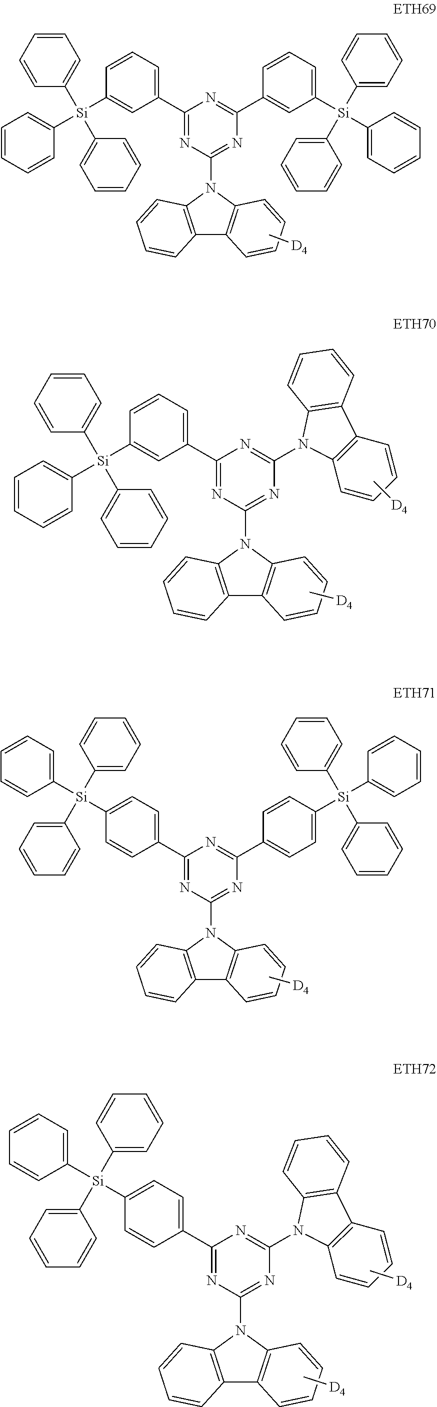

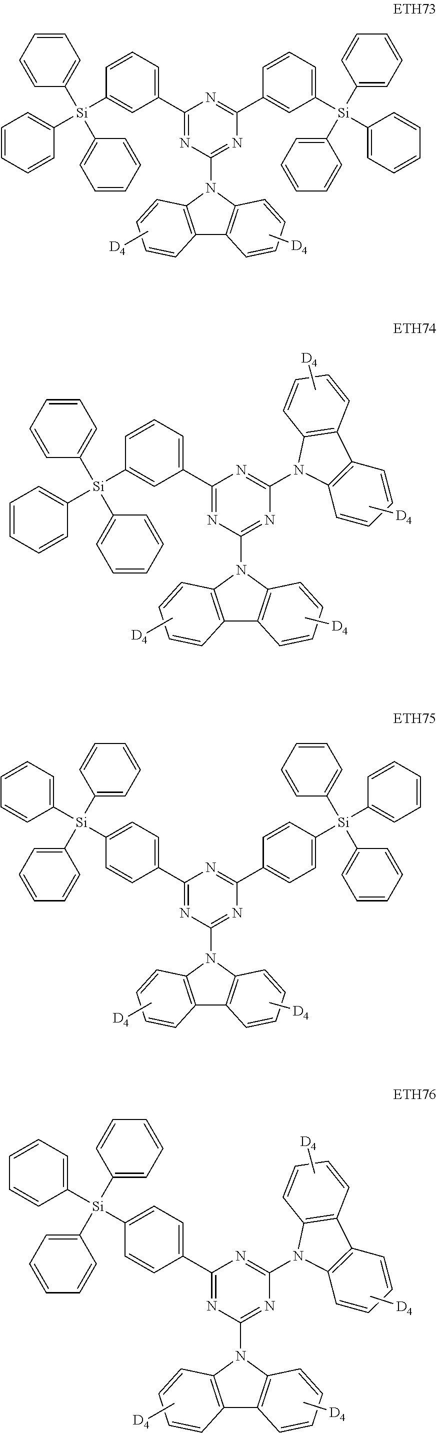

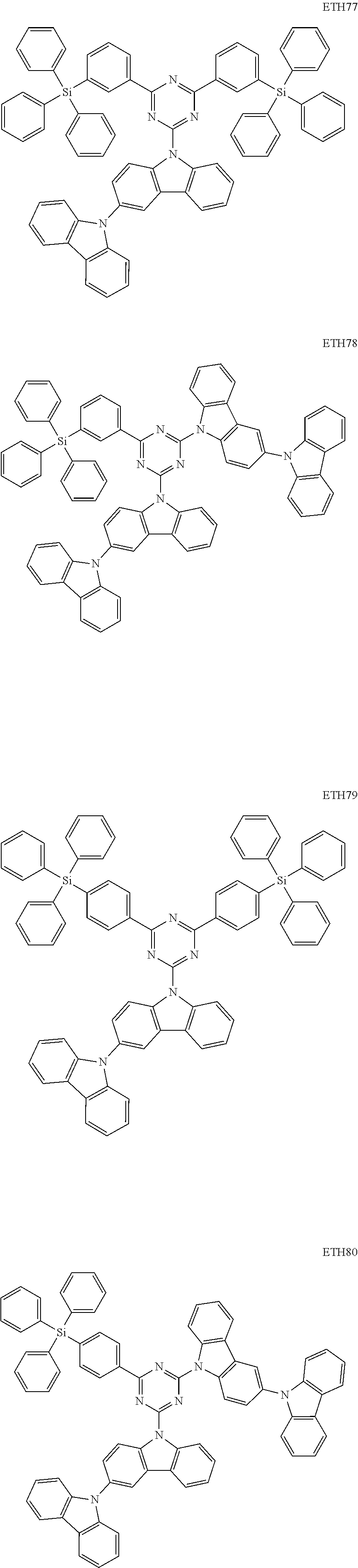

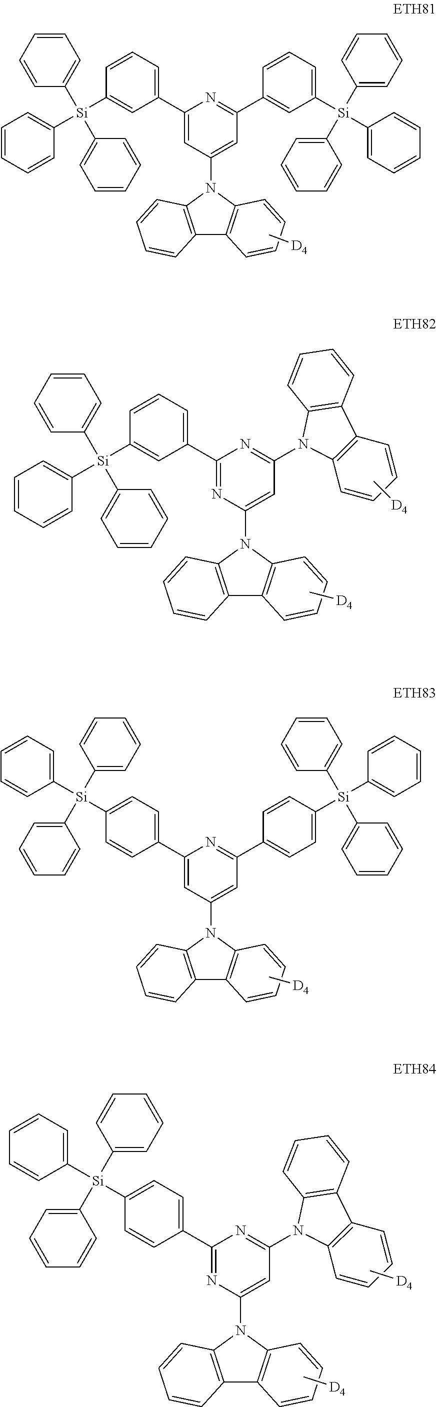

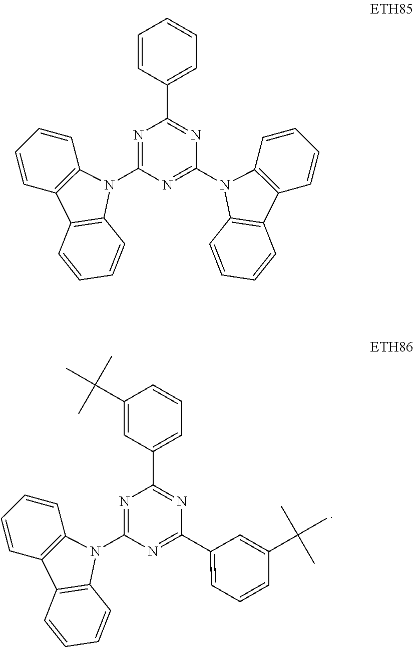 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In the compounds in Compound Group 2, “D” refers to a deuterium atom, and “Ph” refers to an unsubstituted phenyl group.

In one or more embodiments, the emission layer EML may further include a third compound, in addition to the first compound and the second compound. The third compound may be a phosphorescence dopant. In one or more embodiments, the third compound is a light-emitting dopant emitting (e.g., capable of emitting) blue light, and the emission layer EML may be to emit phosphorescence. In one or more embodiments, the emission layer EML may be to emit phosphorescence of blue light. The emission layer EML may include an organometallic complex including platinum (Pt) as a central metal atom and ligands bonded to the central metal atom, as the third compound. In the light emitting device ED of one or more embodiments, the emission layer EML may include a compound represented by Formula D-1 as the third compound.

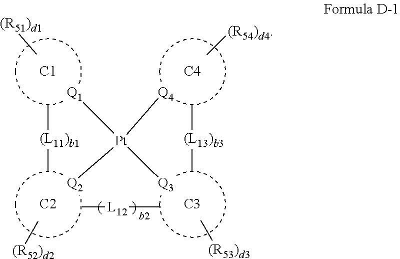

In Formula D-1, Q₁ to Q₄ may each independently be C or N.

In Formula D-1, C1 to C4 may each independently be a substituted or unsubstituted hydrocarbon ring of 5 to 30 ring-forming carbon atoms, or a substituted or unsubstituted heterocycle of 2 to 30 ring-forming carbon atoms.

In Formula D-1, L₁₁ to L₁₃ may each independently be a direct linkage,

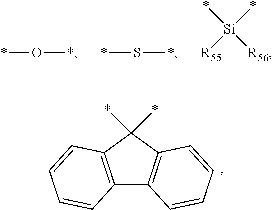

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “ [symbol] ” may refer to a part connected with corresponding ones of C1 to C4.

In Formula D-1, b1 to b3 may each independently be 0 or 1. When b1 is 0, C1 and C2 may not be connected with each other. When b2 is 0, C2 and C3 may not be connected with each other. When b3 is 0, C3 and C4 may not be connected with each other.

In Formula D-1, R₅₁ to R₅₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In some embodiments, R₅₁ to R₅₆ may each independently be combined with an adjacent group to form a ring. R₅₁ to R₅₆ may each independently be a substituted or unsubstituted methyl group, or a substituted or unsubstituted t-butyl group.

In Formula D-1, d1 to d4 may each independently be an integer of 0 to 4. In Formula D-1, when d1 to d4 are 0, the fourth compound may be unsubstituted with R₅₁ to R₅₄, respectively. A case where d1 to d4 are 4, and R₅₁ to R₅₄ are hydrogen atoms, may be the same as a case where d1 to d4 are 0. When d1 to d4 are integers of 2 or more, each of multiple R₅₁ to R₅₄ may be all the same, or at least one selected from among multiple R₅₁ to R₅₄ may be different.

In Formula D-1, C1 to C4 may each independently be a substituted or unsubstituted hydrocarbon ring or a substituted or unsubstituted heterocycle, represented by any one selected from among C-1 to C-4.

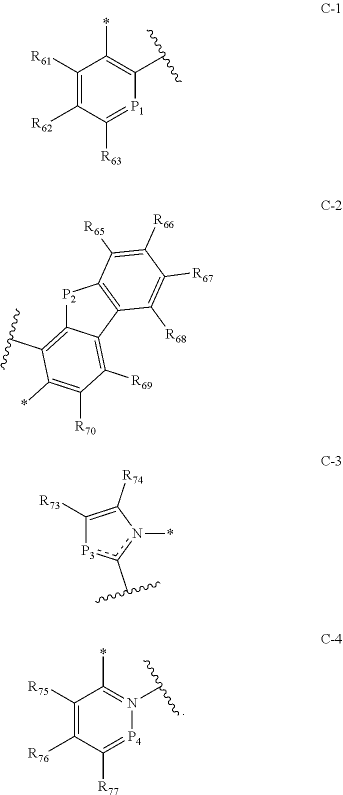

In C-1 to 0-4, P₁ may be “C—*” or CR₆₄, P₂ may be “N—*” or NR₇₁, P₃ may be, “N—*” or NR₇₂, and P4 may be “C—*” or CR₇₈. R₆₁ to R₇₈ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In some embodiments, in C-1 to C-4,

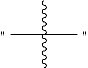

is a part connected with a Pt central metal atom, and “ [symbol] ” is a part connected with neighboring ring groups (C1 to [symbol] C4) and/or linkers (L₁₁ to L₁₃).

In one or more embodiments, the emission layer EML may include at least one selected from among the compounds represented in Compound Group 3 below as the third compound. The emission layer EML may include at least one selected from among the compounds represented in Compound Group 3 as a phosphorescence dopant material.

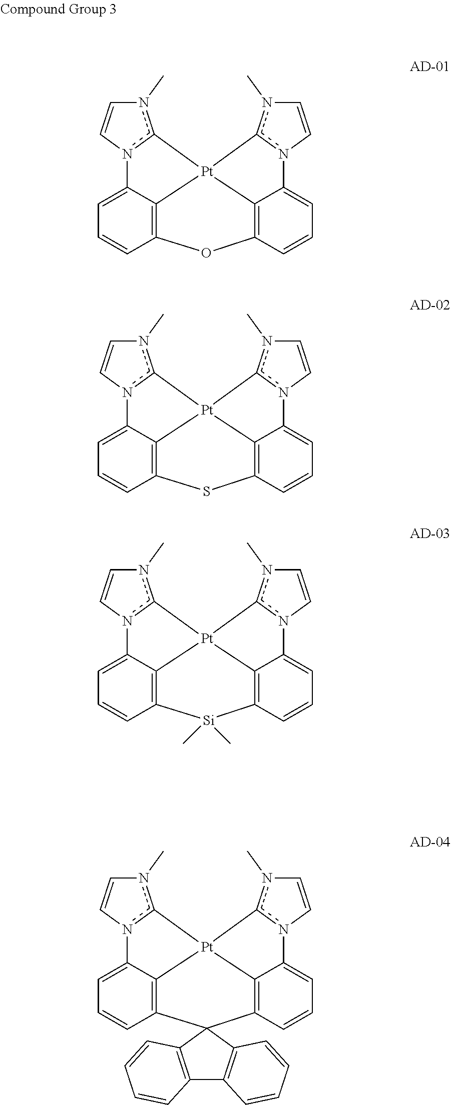

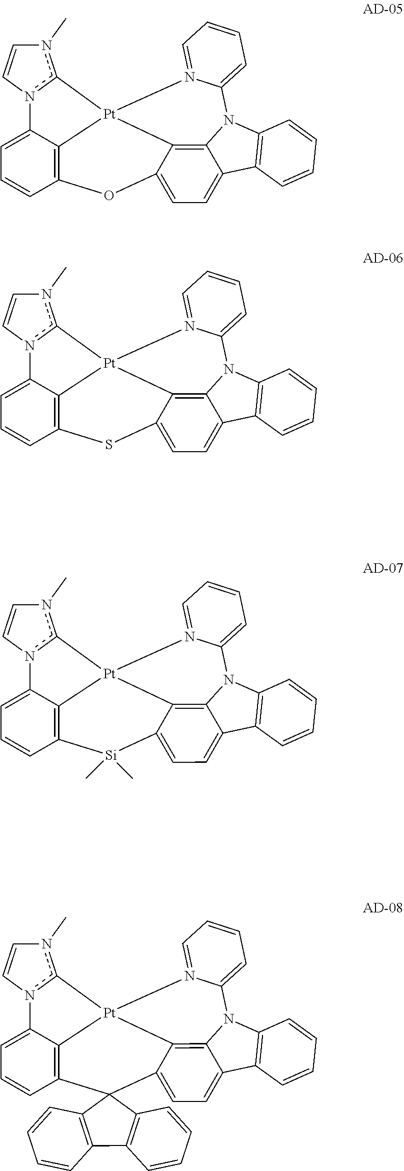

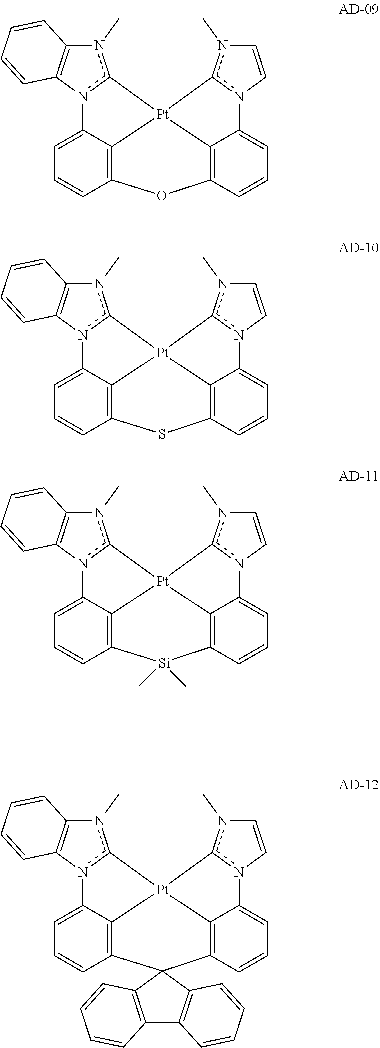

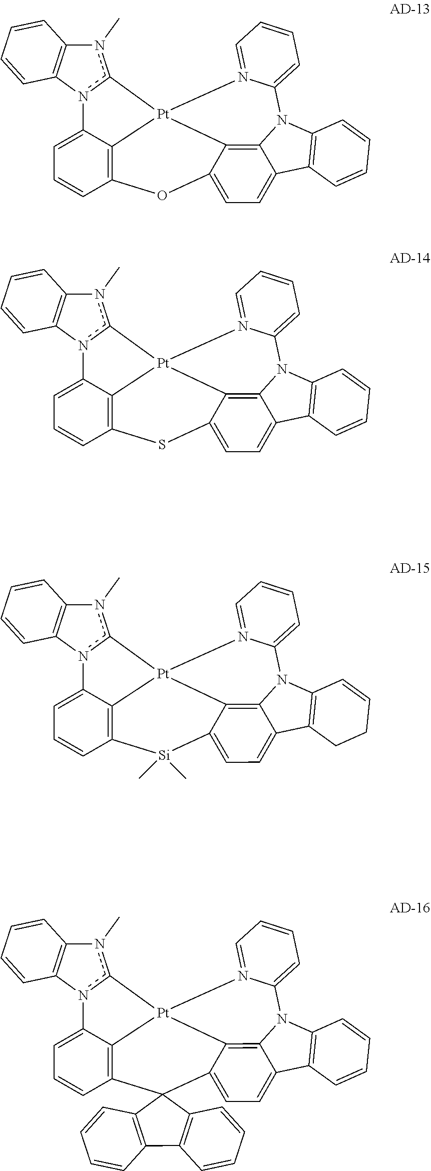

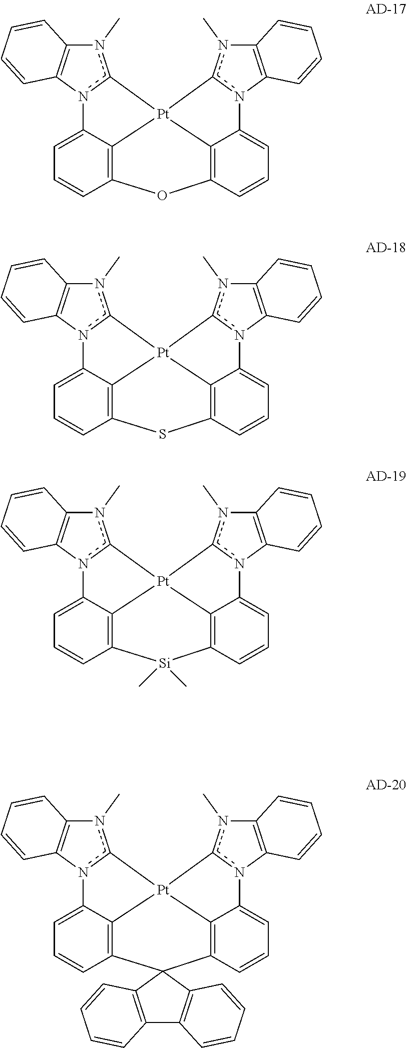

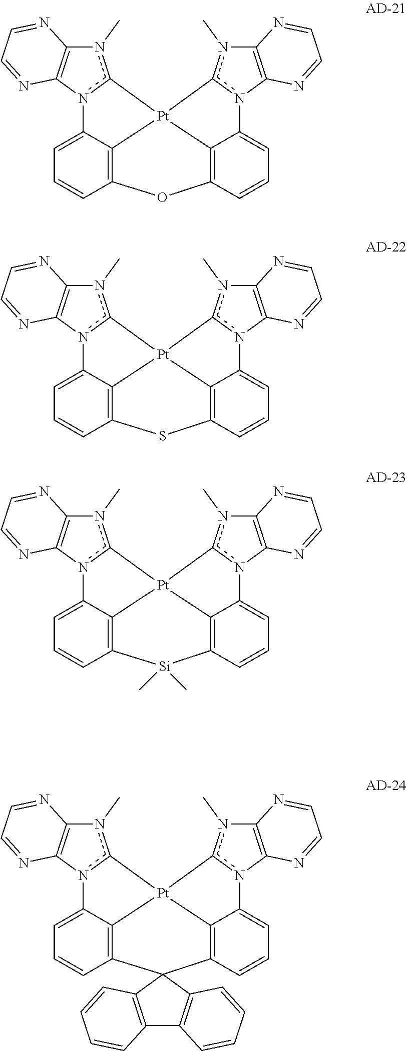

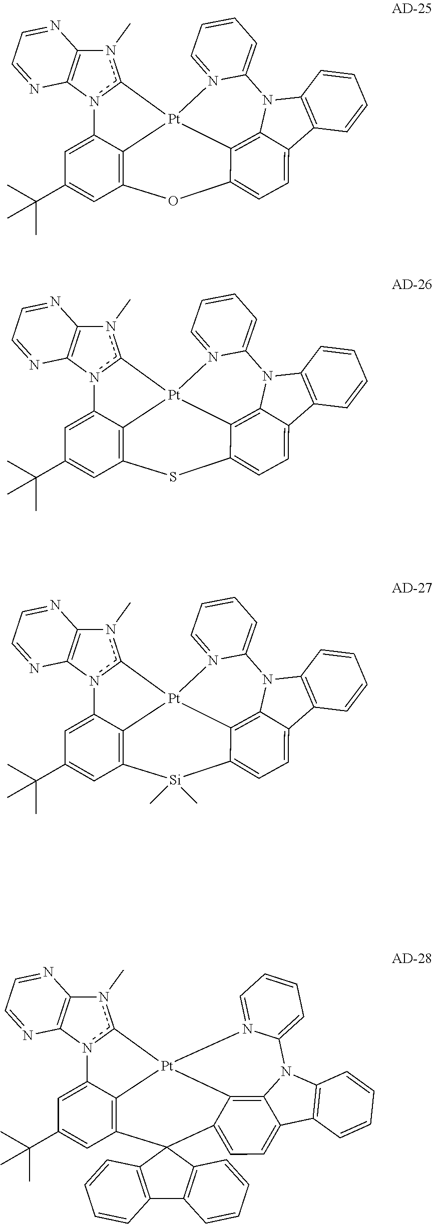

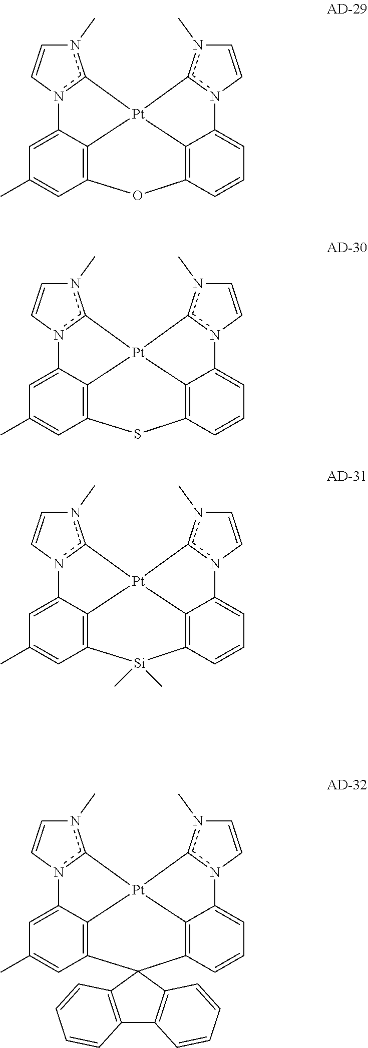

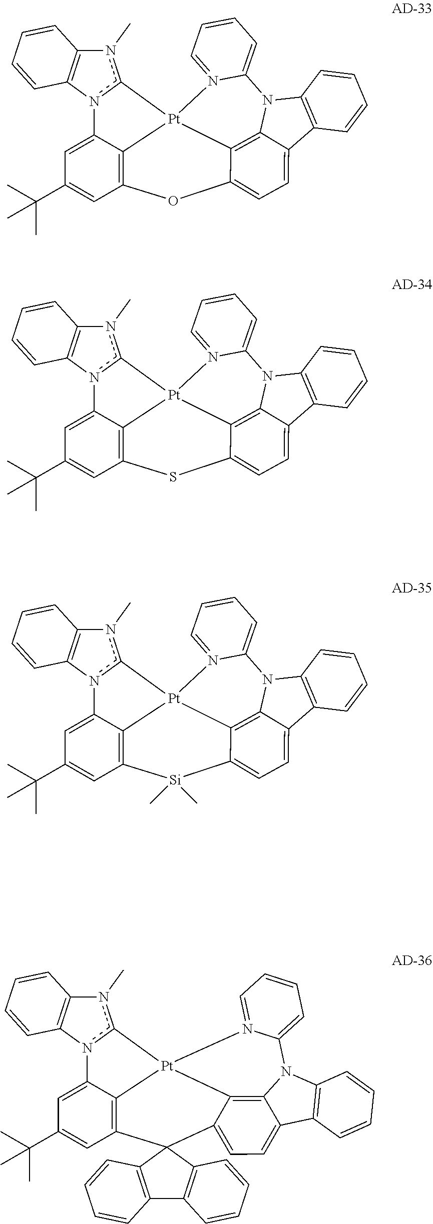

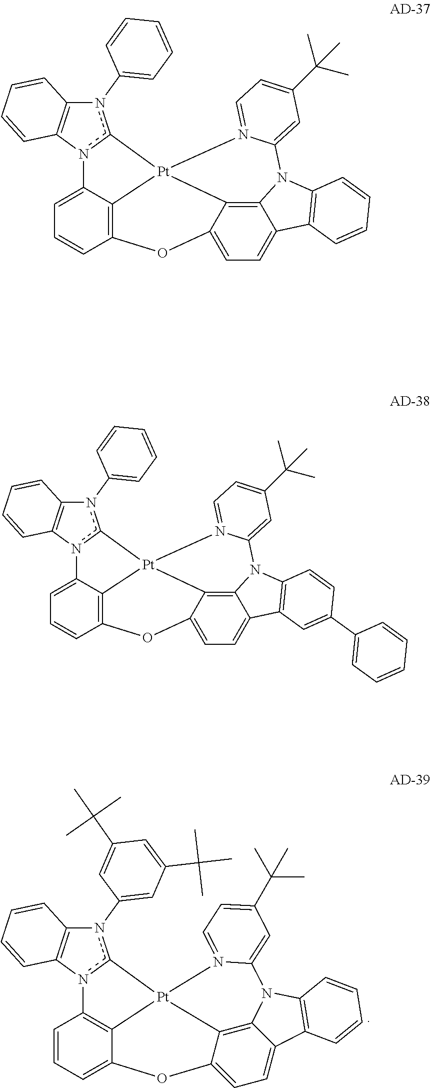

In some embodiments, the light emitting device ED of one or more embodiments may include multiple emission layers. Multiple emission layers may be stacked in order and provided. For example, a light emitting device ED including multiple emission layers may be to emit white light. The light emitting device including multiple emission layers may be a light emitting device of a tandem structure.

In some embodiments, when the light emitting device ED includes multiple emission layers, at least one emission layer EML may include all of the first compound, the second compound and the third compound. For example, the emission layer EML may include the first compound that is the nitrogen-containing compound of one or more embodiments, the second compound represented by Formula ET-1, and the third compound represented by Formula D-1. In one or more embodiments, the light emitting device ED including the multiple compounds may show long-life characteristics and improved light color coordinate.

In the light emitting devices ED of embodiments, shown in FIG. 3 to FIG. 6, the emission layer EML may include a suitable host and a suitable dopant, in addition to the aforementioned host and dopant. For example, the emission layer EML may include a compound represented by Formula E-1. The compound represented by Formula E-1 may be utilized as a fluorescence host material.

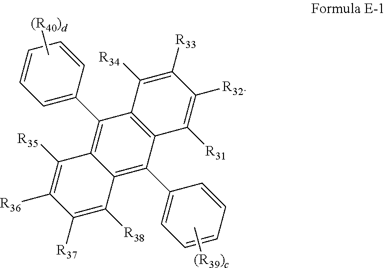

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one selected from among Compound E1 to Compound E19.

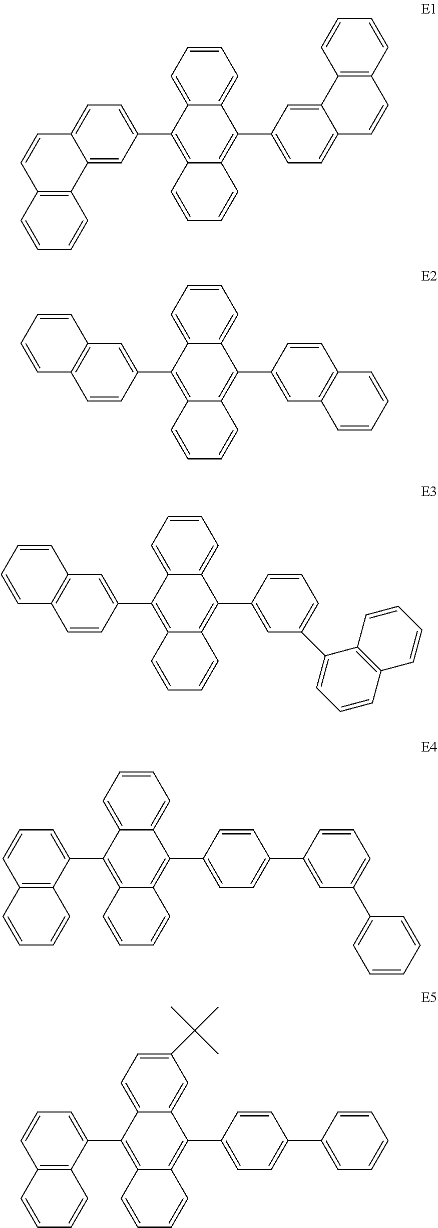

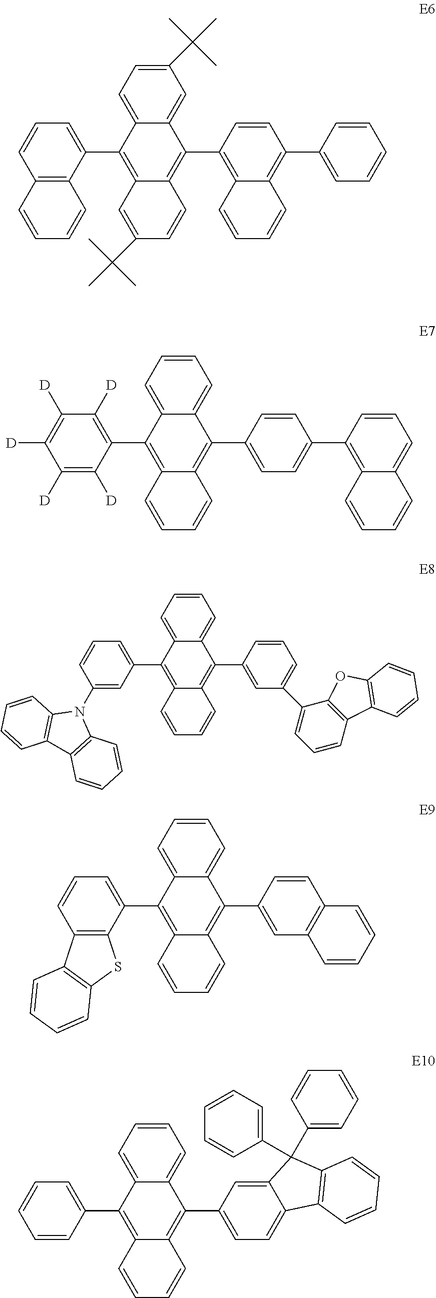

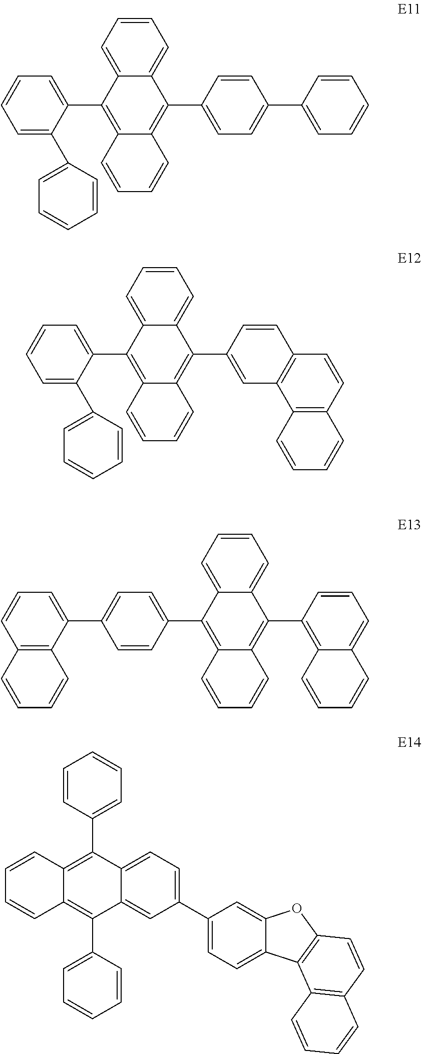

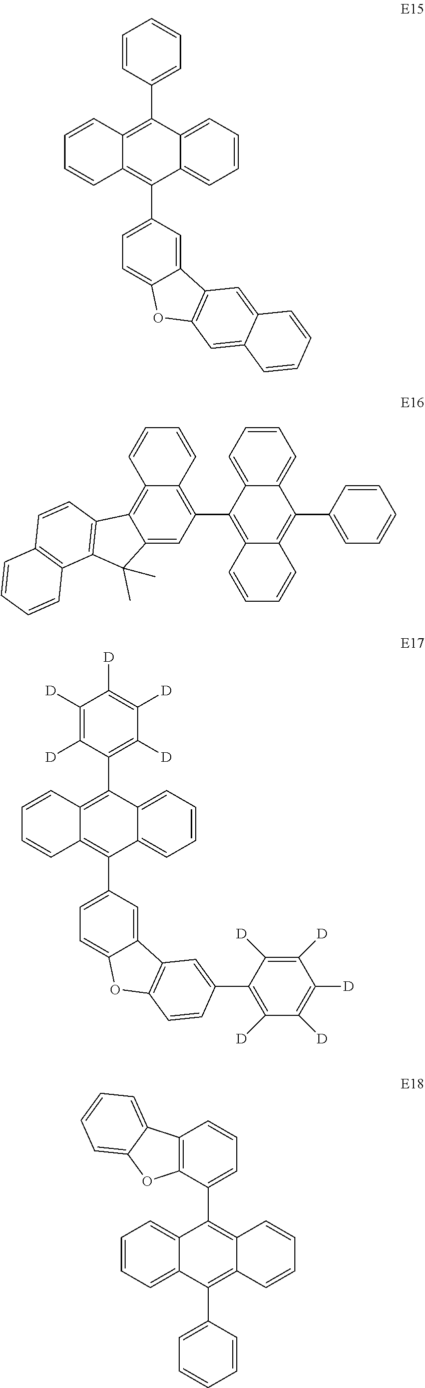

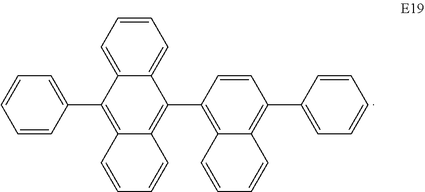 [SMILES: c1ccc(-c2ccc(-c3c4ccccc4c(-c4ccccc4)c4ccccc34)c3ccccc23)cc1]

In one or more embodiments, the emission layer EML may include a compound represented by Formula E-2a or Formula E-2b. The compound represented by Formula E-2a or Formula E-2b may be utilized as a phosphorescence host material.

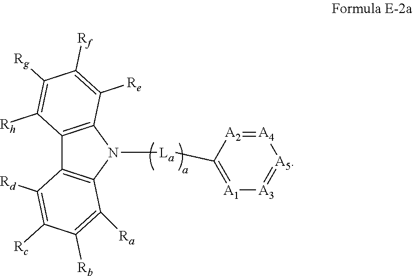

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may each independently be N or CRi. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from among A₁ to A₅ may be N, and the remainder may be CRᵢ.

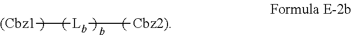

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and when “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds shown in Compound Group E-2 are only illustrations, and the compound represented by Formula E-2a or Formula E-2b is not limited to the compounds represented in Compound Group E-2.

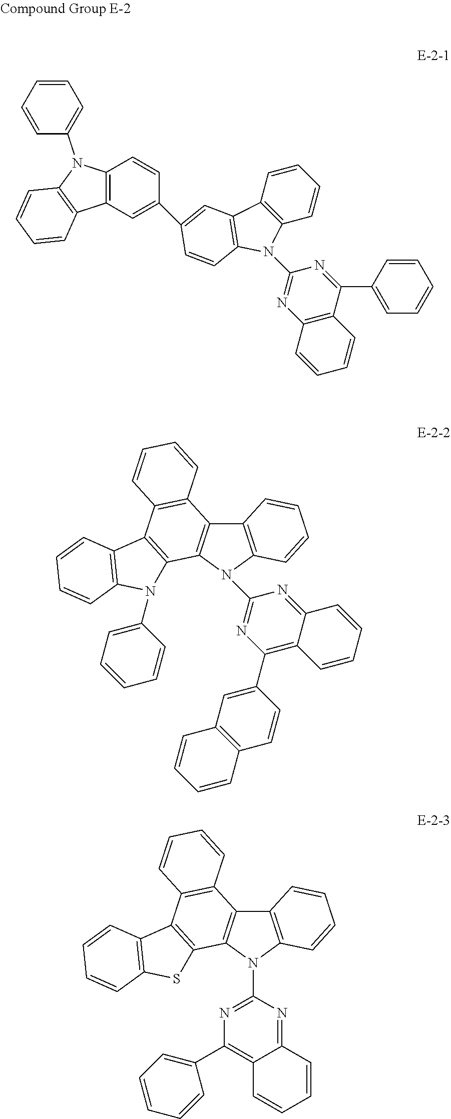

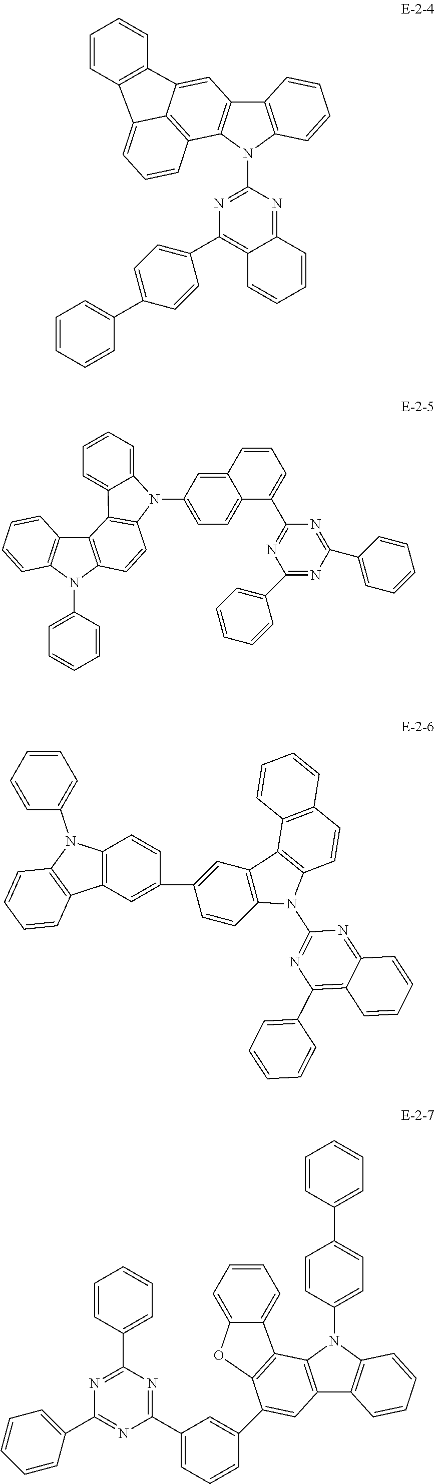 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

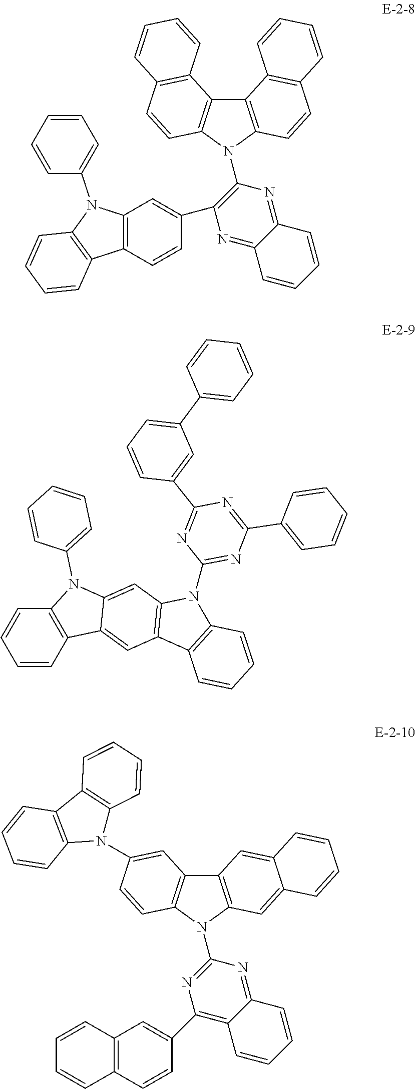 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

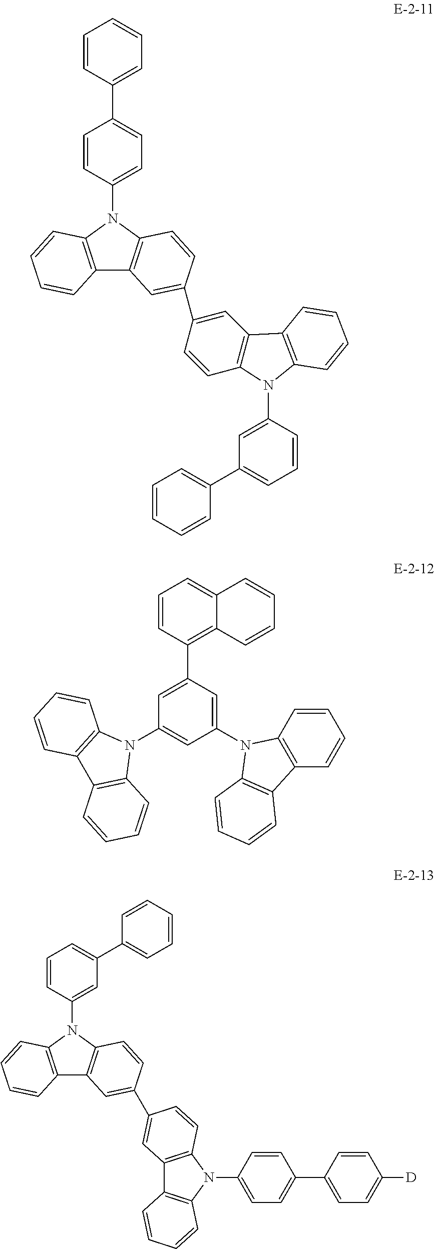

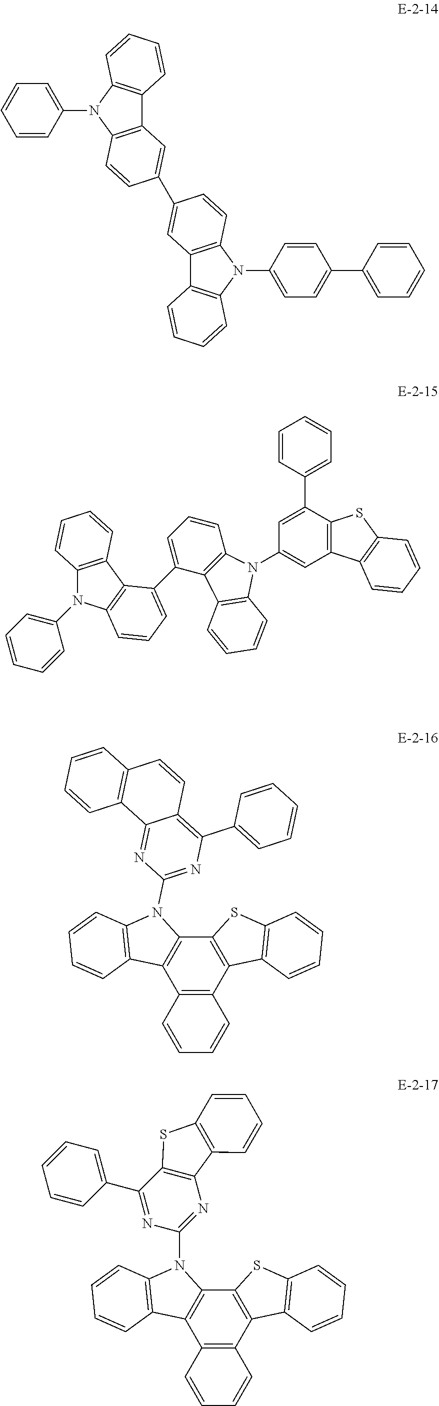

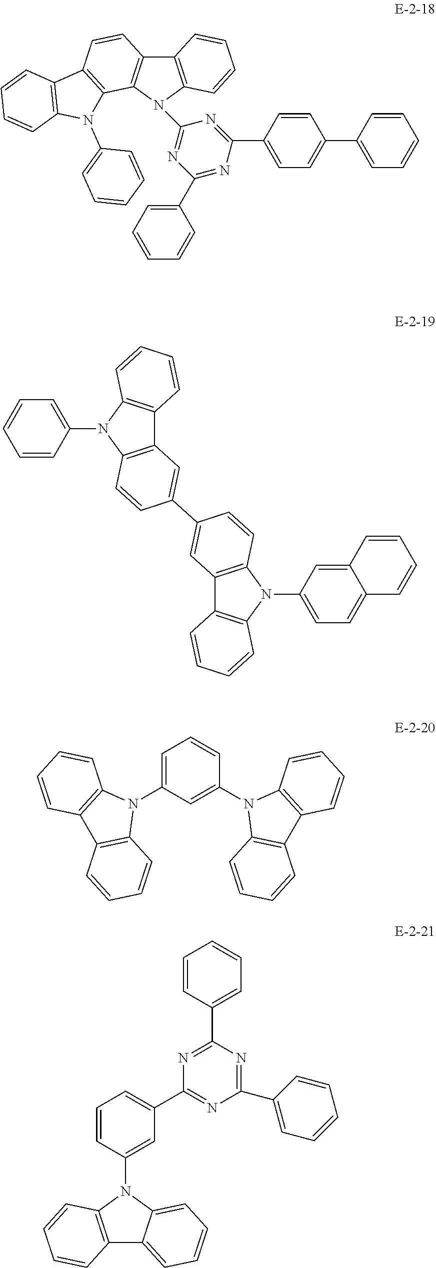 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

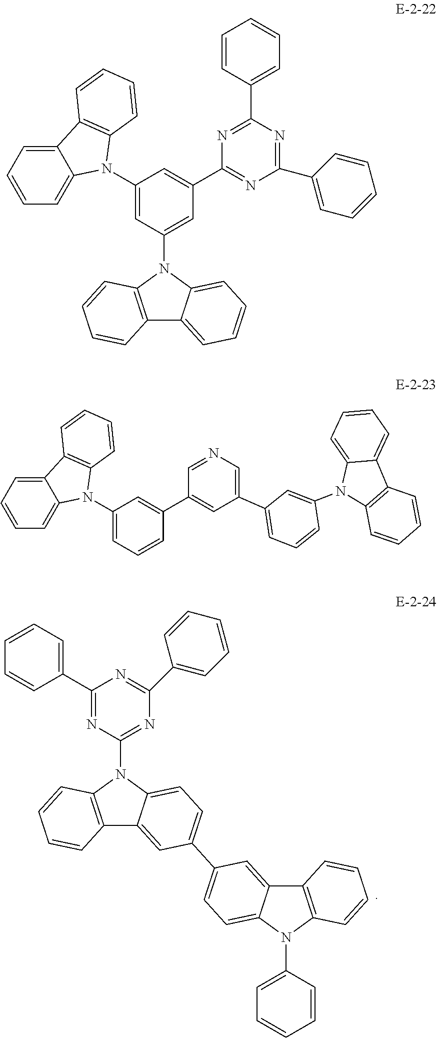 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a suitable host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenyl phosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO3), octaphenylcyclotetra siloxane (DPSiO4), etc. may be utilized as the host material.

The emission layer EML may further include a compound represented by Formula M-a or Formula M-b. The compound represented by Formula M-a or Formula M-b may be utilized as a phosphorescence dopant material. In some embodiments, the compound represented by Formula M-a or Formula M-b may be utilized as an auxiliary dopant material.

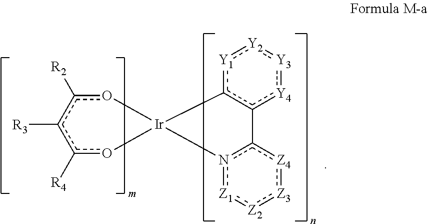

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is 0 or 1, and “n” is 2 or 3. In Formula M-a, when “m” is 0, “n” is 3, and when “m” is 1, “n” is 2.

The compound represented by Formula M-a may be utilized as a phosphorescence dopant.

The compound represented by Formula M-a may be represented by any one selected from among Compounds M-a1 to M-a25. However, Compounds M-a1 to M-a25 are illustrations, and the compound represented by Formula M-a is not limited to the compounds represented by Compounds M-a1 to M-a25.

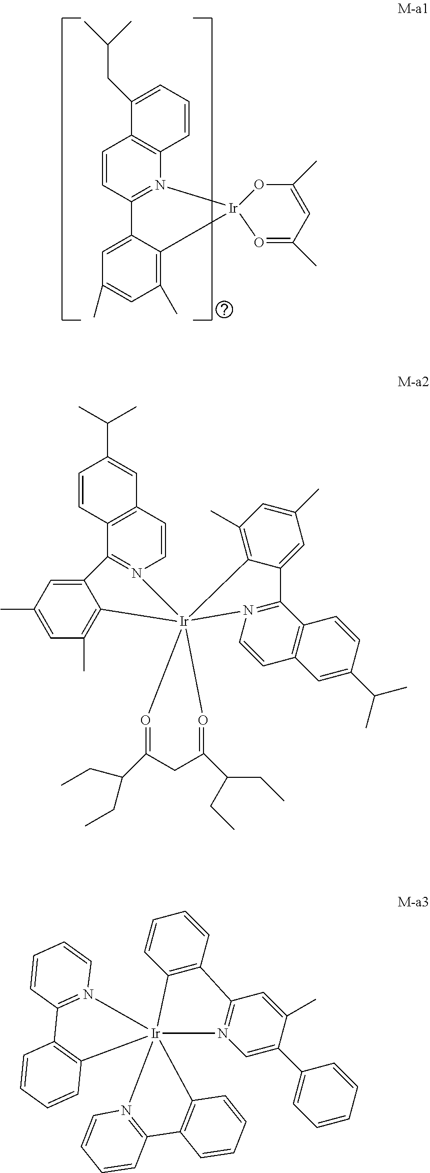

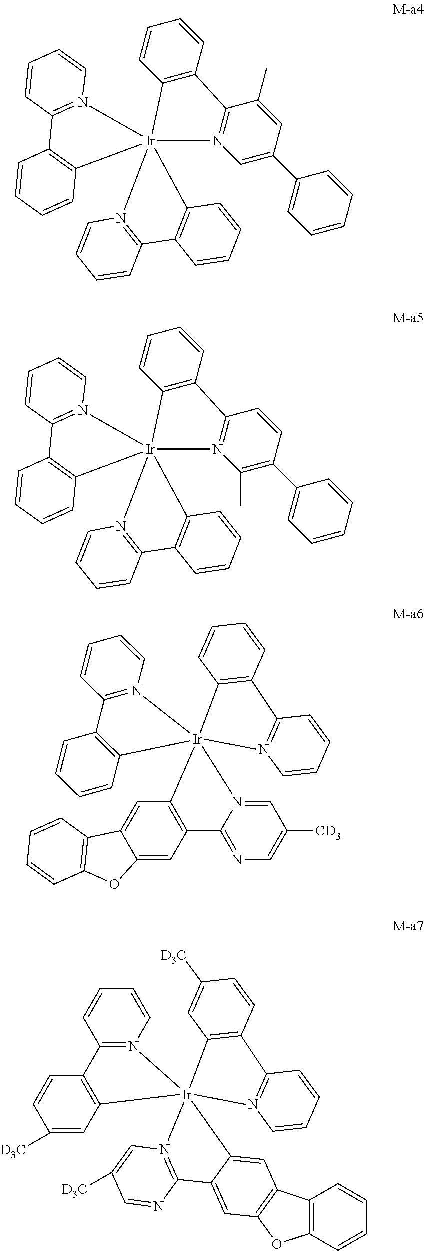

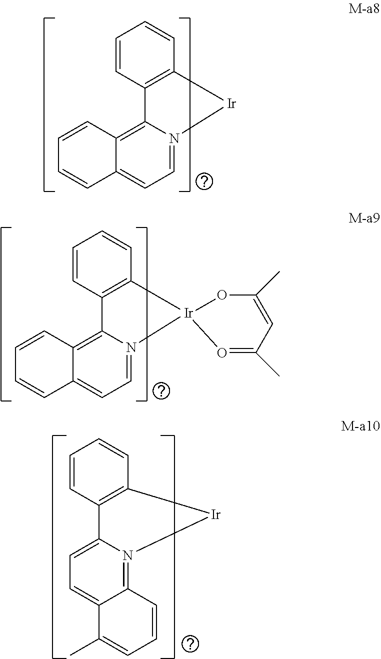

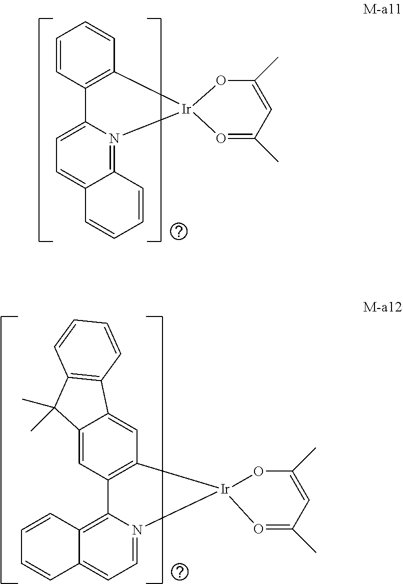

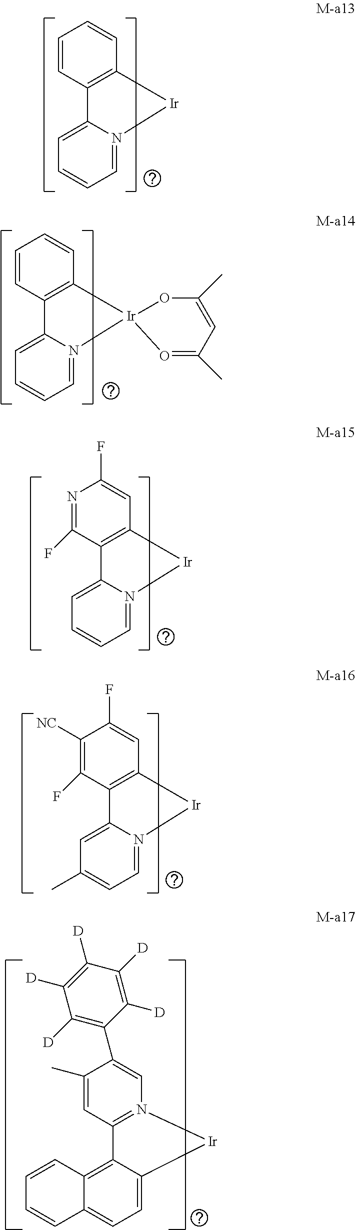

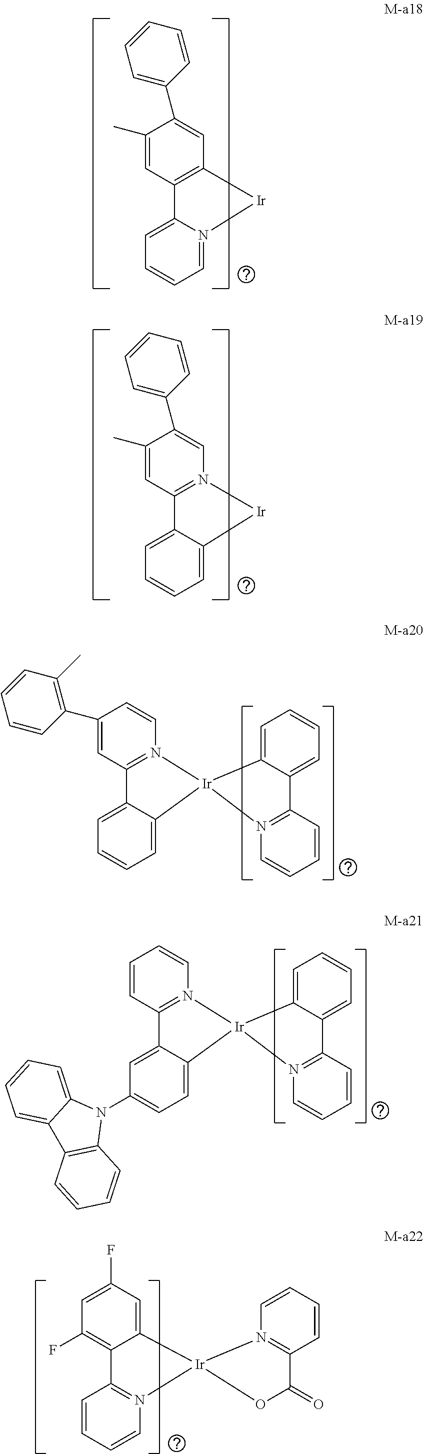

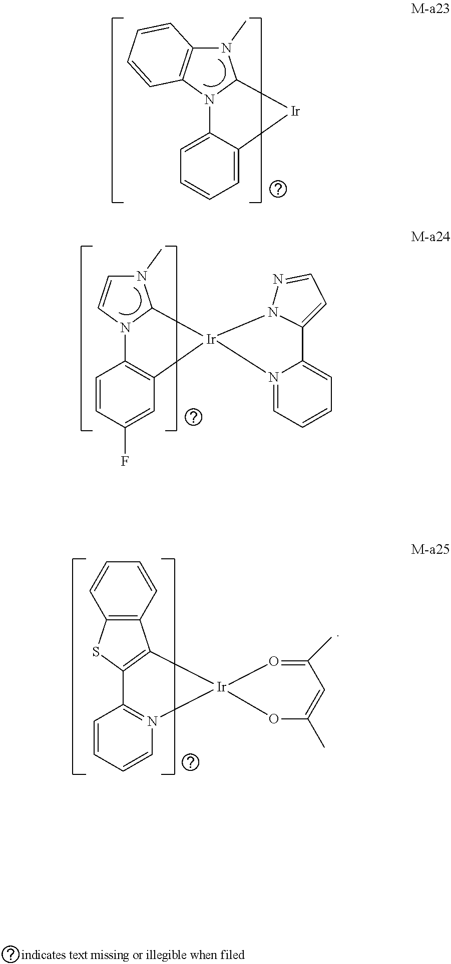

The emission layer EML may include any one selected from among Formula F-a to Formula F-c. The compounds represented by Formula F-a to Formula F-c may be utilized as fluorescence dopant materials.

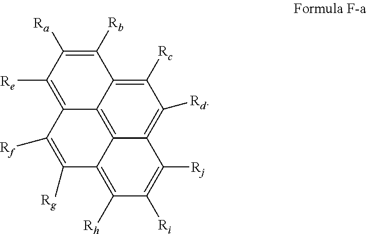

In Formula F-a, two selected from among Rₐ to Rⱼ may each independently be substituted with *—NAr₁Ar₂. The remainder not substituted with *—NAr₁Ar₂ selected from among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including O or S as a ring-forming atom.

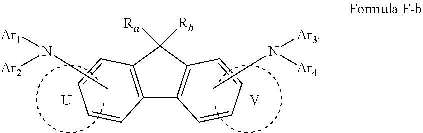

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Ar₁ to Ar₄ may each independently be an aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms. At least one selected from among Ar₁ to Ar₄ may be a heteroaryl group including O or S as a ring-forming atom.

In Formula F-b, the number of rings represented by U and V may each independently be 0 or 1. For example, in Formula F-b, when the number of U or V is 1, one ring forms a fused ring at the designated part by U or V, and when the number of U or V is 0, a ring is not present at the designated part by U or V. For example, when the number of U is 0, and the number of V is 1, or when the number of U is 1, and the number of V is 0, a fused ring having the fluorene core of Formula F-b may be a ring compound with four rings. When the number of both U and V is 0, the fused ring of Formula F-b may be a ring compound with three rings. In some embodiments, when the number of both U and V is 1, a fused ring having the fluorene core of Formula F-b may be a ring compound with five rings.

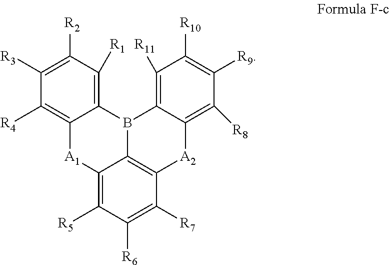

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may each independently be combined with the substituents of an adjacent ring to form a fused ring. For example, when A₁ and A₂ are each independently NRₘ, A₁ may be combined with R₄ or R₅ to form a ring. In some embodiments, A₂ may be combined with R₇ or R₈ to form a ring.

In one or more embodiments, the emission layer EML may include as a suitable dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and/or 4,4′-bis[2-(4-(N,N-diphenylamino)phenyl)vinyl]biphenyl (DPAVBi)), perylene and/or the derivatives thereof (for example, 2,5,8,11-tetra-t-butylperylene (TBP)), pyrene and/or the derivatives thereof (for example, 1,1-dipyrene, 1,4-dipyrenylbenzene, and 1,4-bis(N,N-diphenylamino)pyrene), etc.

The emission layer EML may further include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) and/or thulium (Tm). For example, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), and/or platinum octaethyl porphyrin (PtOEP) may be utilized as the phosphorescence dopant. However, one or more embodiments of the present disclosure is not limited thereto.

The emission layer EML may include a quantum dot material. The core of the quantum dot may be selected from among a II-VI group compound, a Ill-VI group compound, a I-III-VI group compound, a Ill-V group compound, a Ill-II-V group compound, a IV-VI group compound, a IV group element, a IV group compound, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The III-VI group compound may include a binary compound such as In₂S₃ and/or In₂Se₃; a ternary compound such as InGaS₃ and/or InGaSe₃; or optional combinations thereof.

The I-III-VI group compound may be selected from among a ternary compound selected from the group consisting of AgInS, AgInS₂, CuInS, CuInS₂, AgGaS₂, CuGaS₂, CuGaO₂, AgGaO₂, AgAlO₂ and mixtures thereof; and a quaternary compound such as AgInGaS₂ and/or CulnGaS₂.

The III-V group compound may be selected from the group consisting of a binary compound selected from the group consisting of GaN, GaP, GaAs, GaSb, AlN, AIP, AIAs, AISb, InN, InP, InAs, InSb, and mixtures thereof; a ternary compound selected from the group consisting of GaNP, GaNAs, GaNSb, GaPAs, GaPSb, AINP, AINAs, AINSb, AIPAs, AIPSb, InGaP, InAIP, InNP, InNAs, InNSb, InPAs, InPSb, and mixtures thereof; and a quaternary compound selected from the group consisting of GaAINP, GaAINAs, GaAINSb, GaAIPAs, GaAIPSb, GaInNP, GaInNAs, GalnNSb, GaInPAs, GalnPSb, InAINP, InAINAs, InAINSb, InAIPAs, InAIPSb, and mixtures thereof. In some embodiments, the III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof; and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In this case, the binary compound, the ternary compound and/or the quaternary compound may be present at substantially uniform concentration in a particle or may be present at a partially different concentration distribution state in substantially the same particle. In some embodiments, a core/shell structure in which one quantum dot wraps another quantum dot may be possible. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal oxide, a non-metal oxide, a semiconductor compound, and combinations thereof.

For example, the metal oxide and/or the non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO; and/or a ternary compound such as MgAl₂O₄, CoFe₂O₄, NiFe₂O₄ and/or CoMn₂O₄, but one or more embodiments of the present disclosure is not limited thereto.

In some embodiments, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AIAs, AIP, AISb, etc., but one or more embodiments of the present disclosure is not limited thereto.

The quantum dot may have a full width of half maximum (FWHM) of emission wavelength spectrum of about 45 nm or less, for example, about 40 nm or less, or about 30 nm or less. Within any of these ranges, color purity and/or color reproducibility may be improved. In some embodiments, light emitted via such quantum dot is emitted in all directions, and light view angle properties may be improved.

In some embodiments, the shape of the quantum dot may be any suitable shape in the art, without specific limitation. For example, a spherical, pyramidal, multi-arm, and/or cubic nanoparticle, nanotube, nanowire, nanofiber, nanoplate particle, etc. may be utilized.

The quantum dot may control the color of light emitted according to the particle size, and accordingly, the quantum dot may have one or more suitable emission colors such as blue, red and/or green.

In the light emitting devices ED of embodiments, as shown in FIG. 3 to FIG. 6, the electron transport region ETR is provided on the emission layer EML. The electron transport region ETR may include at least one of a hole blocking layer HBL, an electron transport layer ETL, or an electron injection layer EIL. However, one or more embodiments of the present disclosure is not limited thereto.

The electron transport region ETR may have a single layer formed utilizing a single material, a single layer formed utilizing multiple different materials, or a multilayer structure having multiple layers formed utilizing multiple different materials.

For example, the electron transport region ETR may have a single layer structure of an electron injection layer EIL or an electron transport layer ETL, or a single layer structure formed utilizing an electron injection material and an electron transport material. Further, the electron transport region ETR may have a single layer structure formed utilizing multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-2.

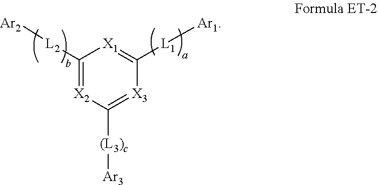

In Formula ET-2, at least one selected from among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are integers of 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, one or more embodiments of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and/or mixtures thereof, without limitation.

The electron transport region ETR may include at least one selected from among Compounds ET1 to ET36.

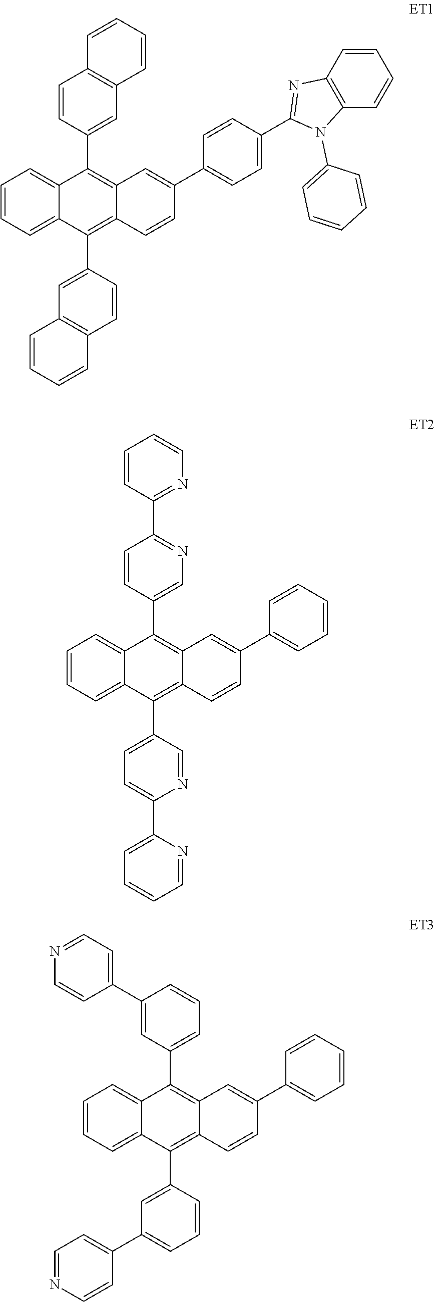 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

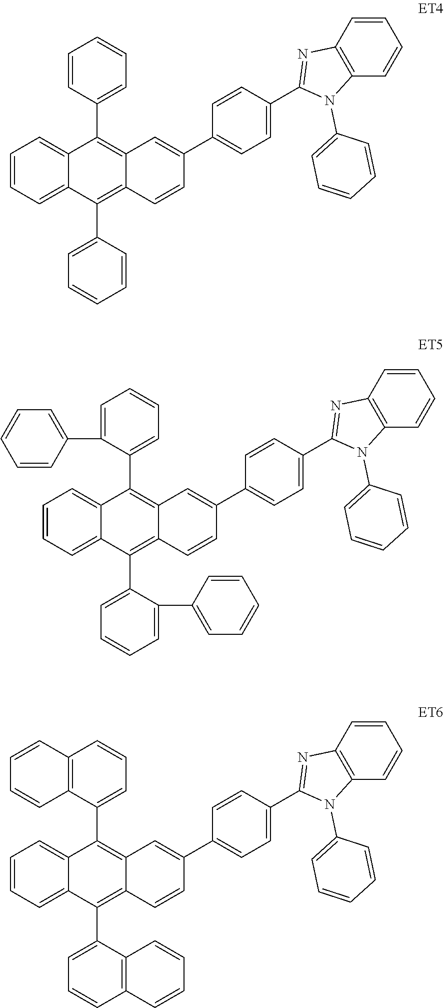 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

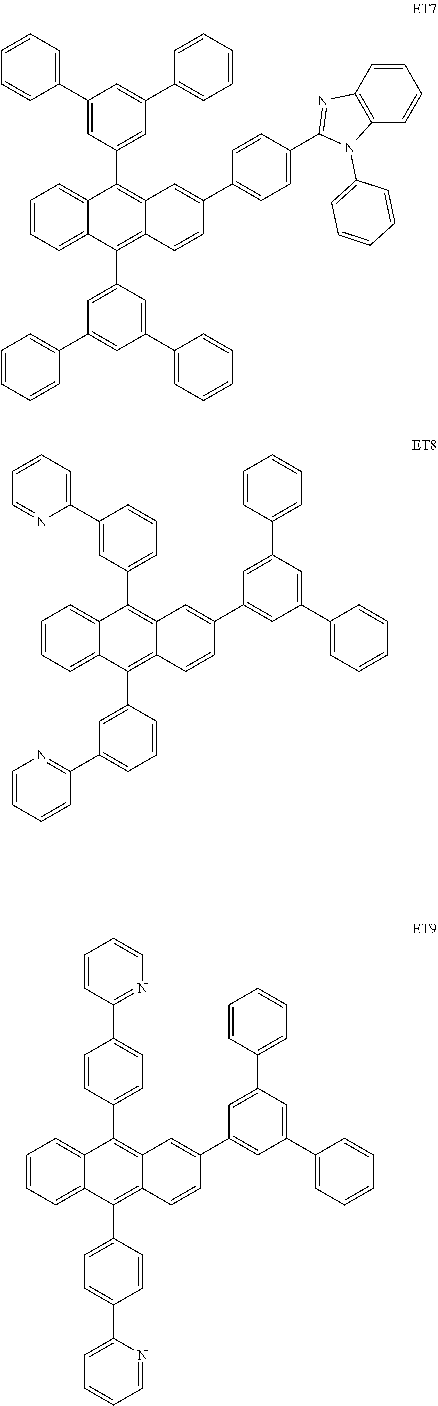 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

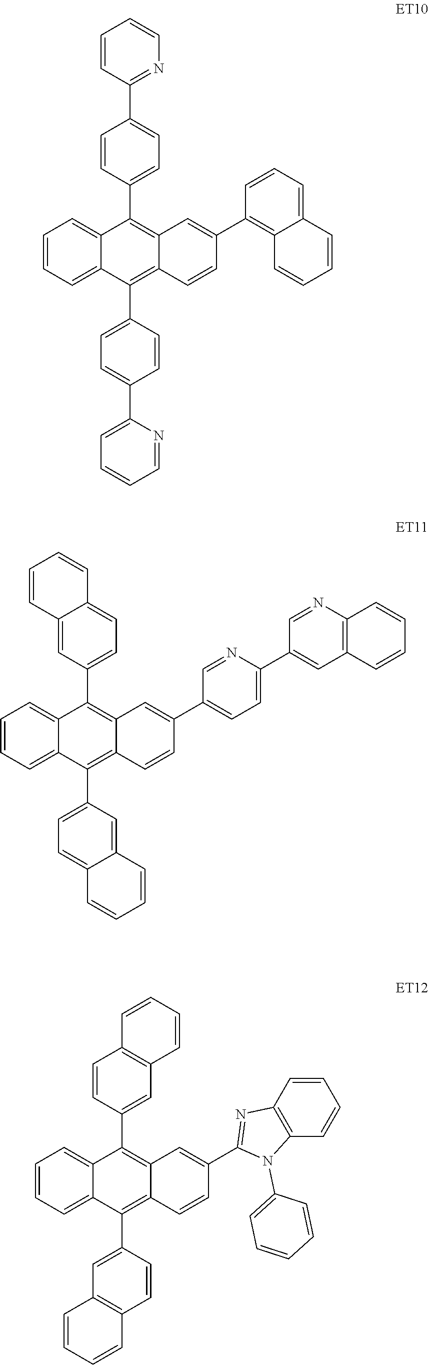 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

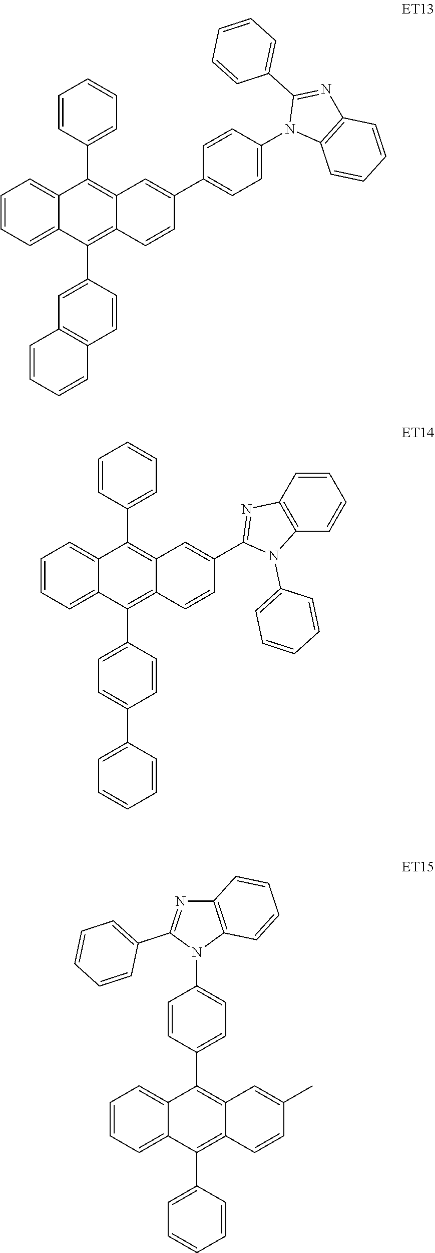 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

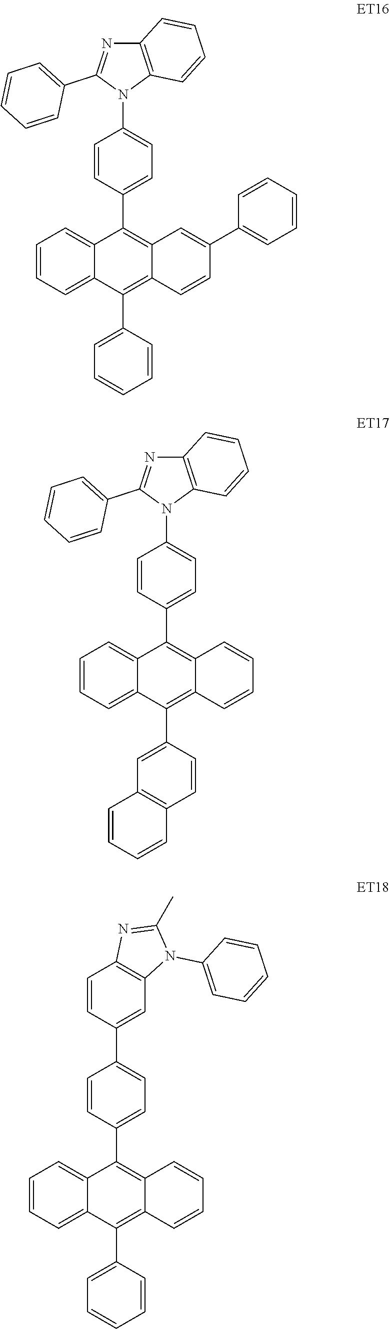 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

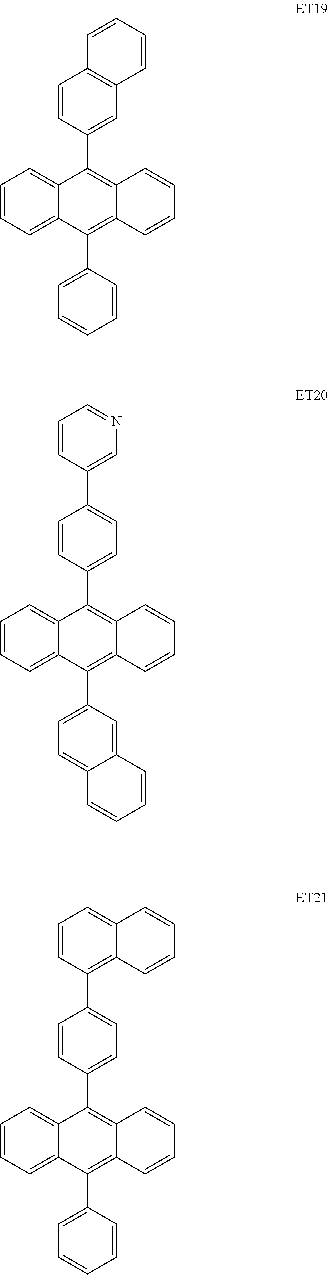

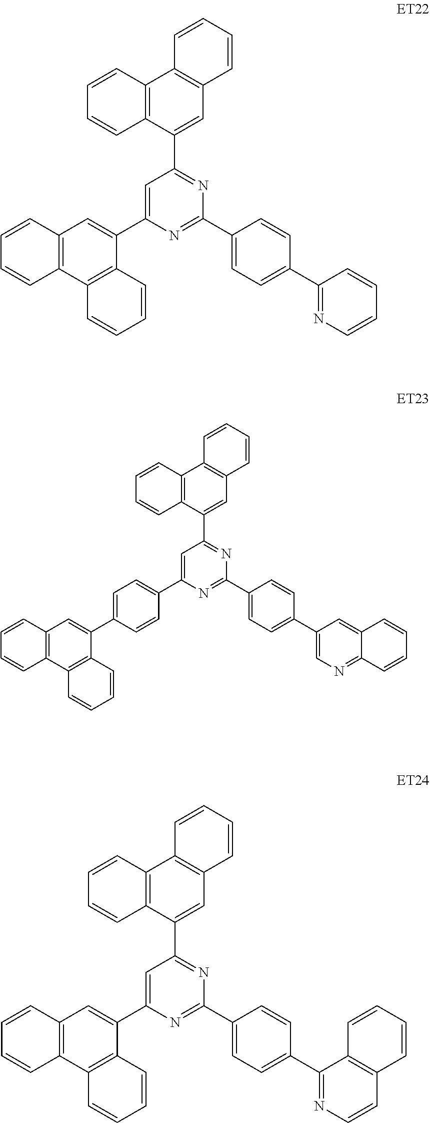 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

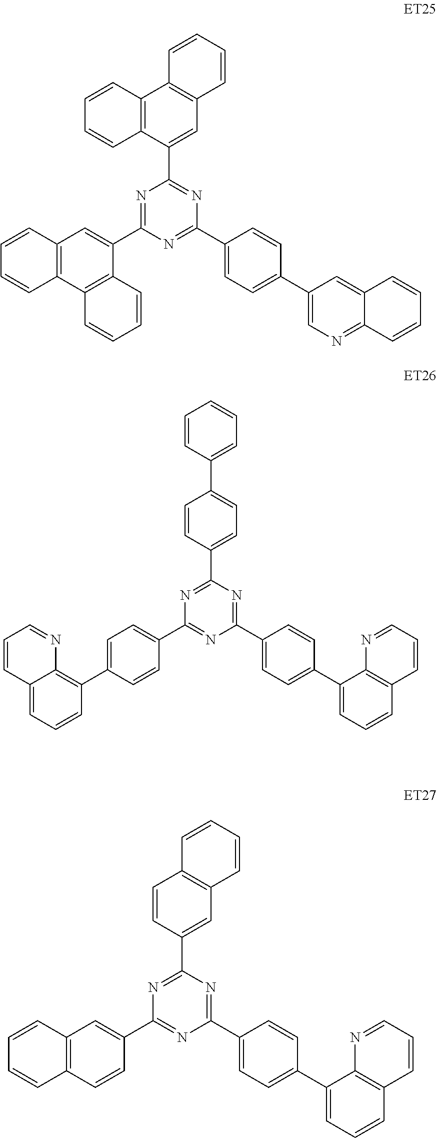 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

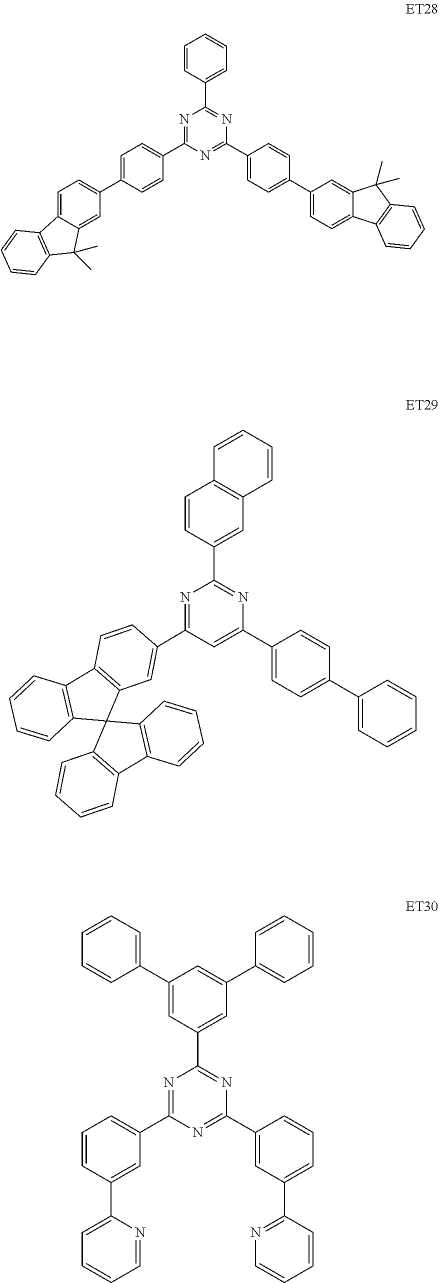 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

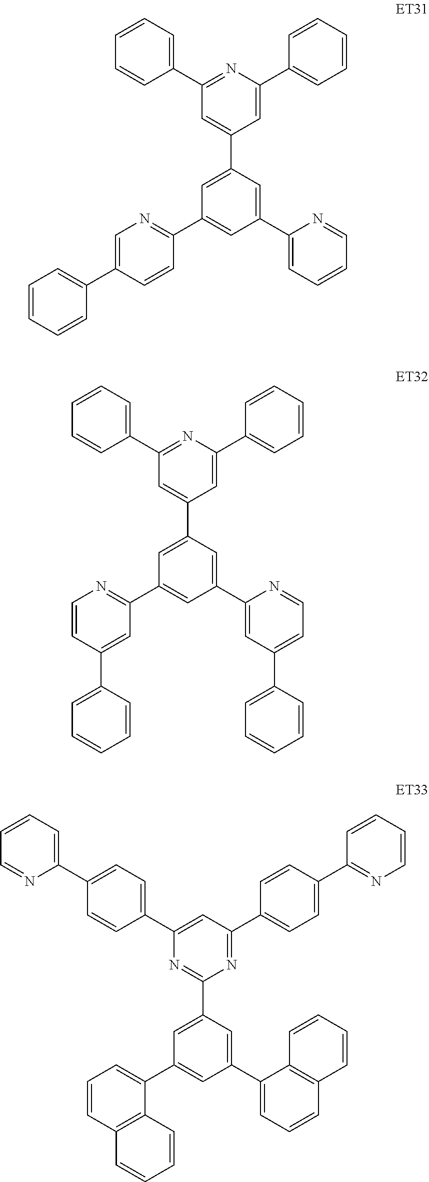 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

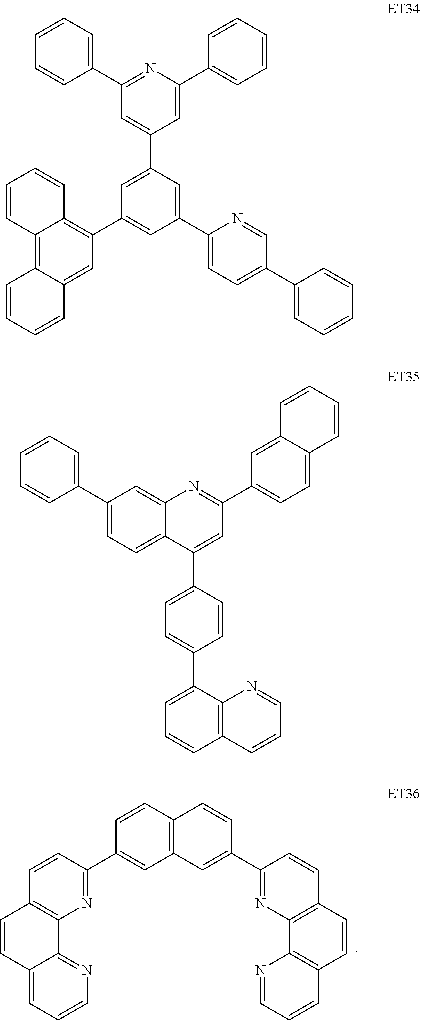 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI and/or KI, a lanthanide metal such as Yb, or a co-depositing material of the metal halide and the lanthanide metal. For example, the electron transport region ETR may include KI:Yb, RbI:Yb, LiF:Yb, etc., as the co-depositing material. In some embodiments, the electron transport region ETR may utilize a metal oxide such as Li₂O and/or BaO, and/or 8-hydroxy-lithium quinolate (Liq). However, one or more embodiments of the present disclosure is not limited thereto. The electron transport region ETR also may be formed utilizing a mixture material of an electron transport material and an insulating organo metal salt. The organo metal salt may be a material having an energy band gap of about 4 eV or more. For example, the organo metal salt may include, for example, one or more of metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen), in addition to the aforementioned materials. However, one or more embodiments of the present disclosure is not limited thereto.

The electron transport region ETR may include the compounds of the electron transport region in at least one selected from among an electron injection layer EIL, an electron transport layer ETL, and a hole blocking layer HBL.

When the electron transport region ETR includes the electron transport layer ETL, the thickness of the electron transport layer ETL may be from about 100 Å to about 1,000 Å, for example, from about 150 Å to about 500 Å. When the thickness of the electron transport layer ETL satisfies any of the above-described ranges, satisfactory or suitable electron transport properties may be obtained without substantial increase of a driving voltage. When the electron transport region ETR includes the electron injection layer EIL, the thickness of the electron injection layer EIL may be from about 1 Å to about 100 Å, and from about 3 Å to about 90 Å. When the thickness of the electron injection layer EIL satisfies any of the above described ranges, satisfactory or suitable electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, when the first electrode EL1 is an anode, the second cathode EL2 may be a cathode, and when the first electrode EL1 is a cathode, the second electrode EL2 may be an anode.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds and/or mixtures including thereof (for example, AgMg, AgYb, and/or MgYb). In some embodiments, the second electrode EL2 may have a multilayered structure including a reflective layer or a transflective layer formed utilizing any of the above-described materials and a transparent conductive layer formed utilizing ITO, IZO, ZnO, ITZO, etc. For example, the second electrode EL2 may include the aforementioned metal materials, combinations of two or more metal materials selected from among the aforementioned metal materials, and/or oxides of the aforementioned metal materials.

In some embodiments, the second electrode EL2 may be connected with an auxiliary electrode. When the second electrode EL2 is connected with the auxiliary electrode, the resistance of the second electrode EL2 may decrease.

In some embodiments, on the second electrode EL2 in the light emitting device ED of one or more embodiments, a capping layer CPL may be further provided. The capping layer CPL may include a multilayer or a single layer.

In one or more embodiments, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiNx, SiOy, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., and/or includes an epoxy resin, and/or acrylate such as methacrylate. In some embodiments, a capping layer CPL may include at least one selected from among Compounds P1 to P5, but one or more embodiments of the present disclosure is not limited thereto.

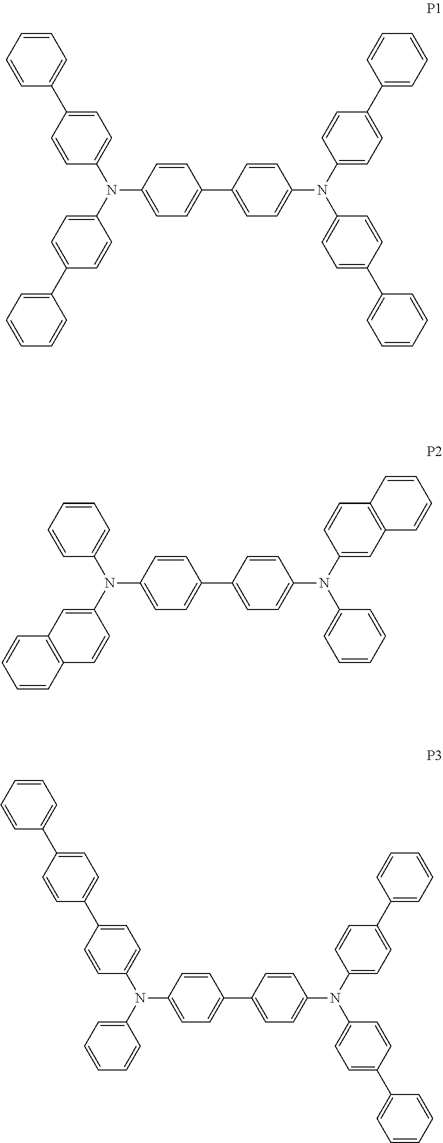 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

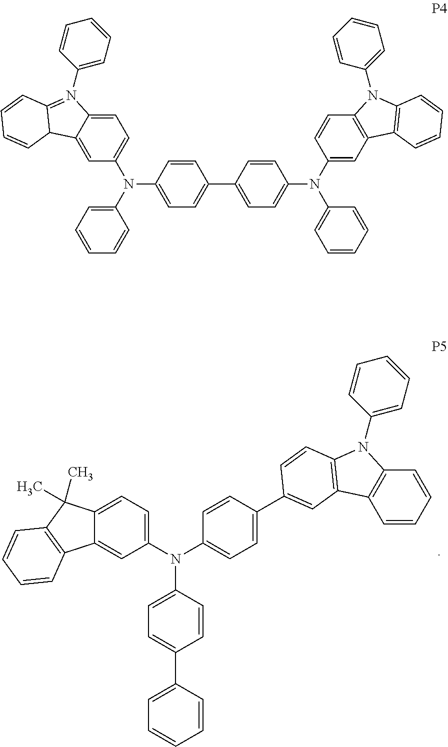

In some embodiments, the refractive index of the capping layer CPL may be about 1.6 or more. For example, the refractive index of the capping layer CPL with respect to light in a wavelength range of about 550 nm to about 660 nm may be about 1.6 or more.

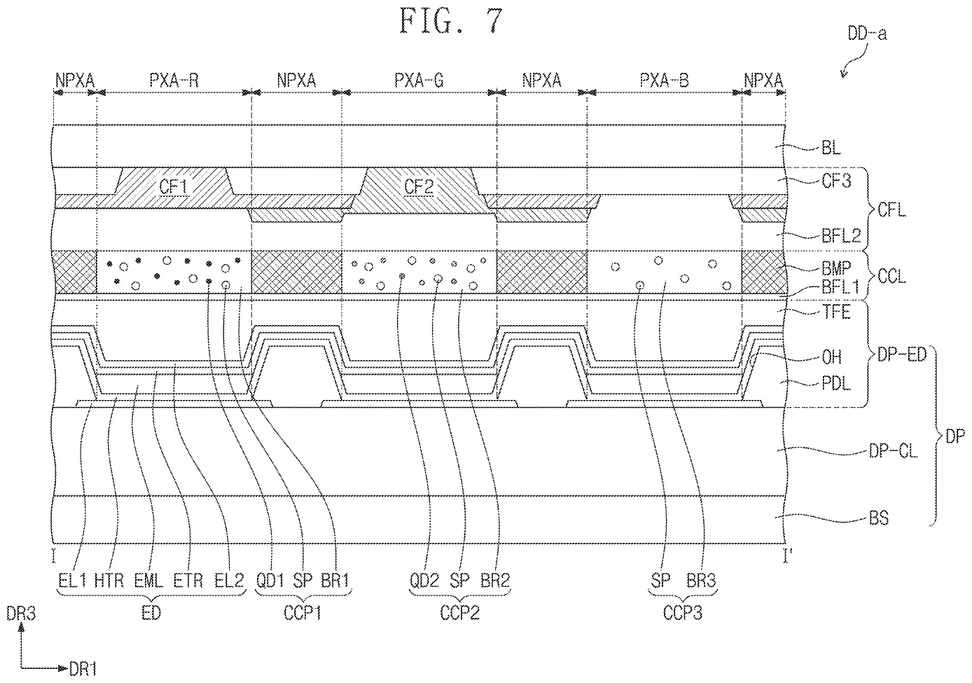

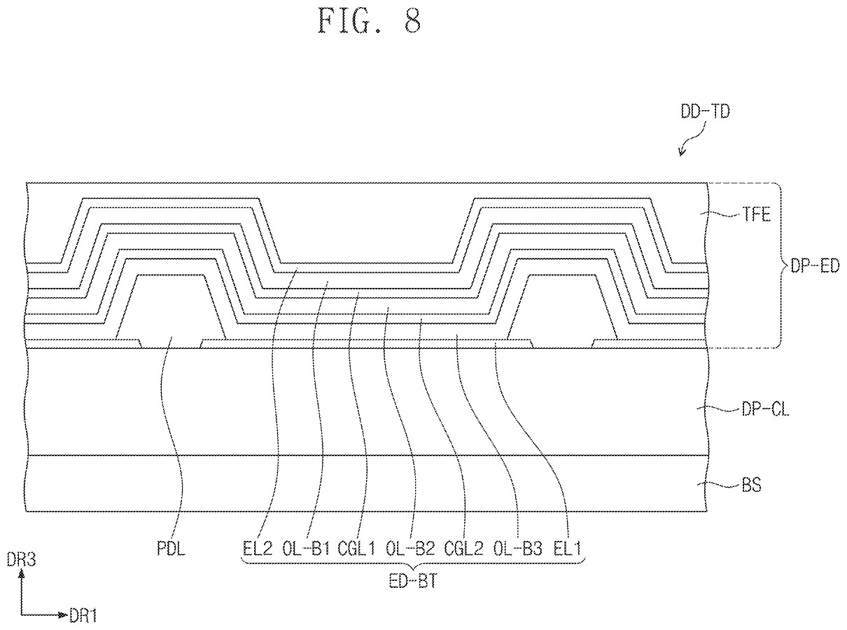

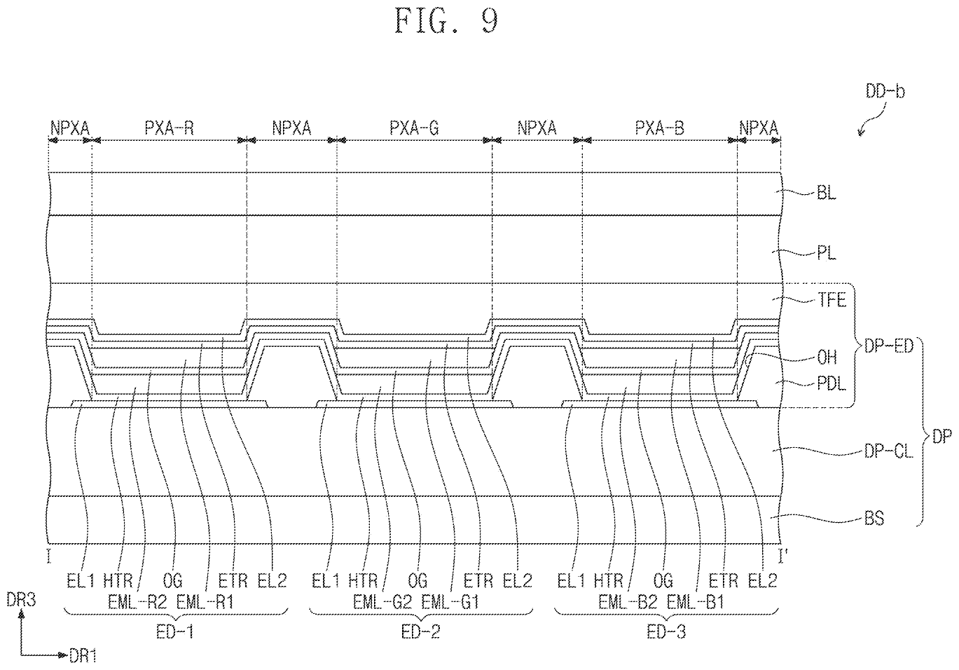

FIG. 7 and FIG. 8 are cross-sectional views on display apparatuses according to embodiments of the present disclosure. In the explanation of the display apparatuses of embodiments, referring to FIG. 7 and FIG. 8, the overlapping content with the explanation on FIG. 1 to FIG. 6 will not be explained again, and the different features will be explained chiefly.

Referring to FIG. 7, a display apparatus DD according to one or more embodiments may include a display panel DP including a display device layer DP-ED, a light controlling layer CCL provided on the display panel DP, and a color filter layer CFL.

In one or more embodiments shown in FIG. 7, the display panel DP includes a base layer BS, a circuit layer DP-CL provided on the base layer BS and a display device layer DP-ED, and the display device layer DP-ED may include a light emitting device ED.

The light emitting device ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. In some embodiments, the same structures of the light emitting devices of FIG. 3 to FIG. 6 may be applied to the structure of the light emitting device ED shown in FIG. 7.

The emission layer EML of a light emitting device ED included in a display apparatus DD-a according to one or more embodiments, may include the nitrogen-containing compound of one or more embodiments.

Referring to FIG. 7, the emission layer EML may be provided in an opening part OH defined in a pixel definition layer PDL. For example, the emission layer EML divided by the pixel definition layer PDL and correspondingly provided to each of luminous areas PXA-R, PXA-G and PXA-B may be to emit light in substantially the same wavelength region. In the display apparatus DD-a of one or more embodiments, the emission layer EML may be to emit blue light. In some embodiments, the emission layer EML may be provided as a common layer for all luminous areas PXA-R, PXA-G and PXA-B.

The light controlling layer CCL may be provided on the display panel DP. The light controlling layer CCL may include a light converter. The light converter may be a quantum dot and/or a phosphor. The light converter may transform the wavelength of light provided and then emit the transformed (converted) light. For example, the light controlling layer CCL may be a layer including a quantum dot and/or a layer including a phosphor.

The light controlling layer CCL may include light controlling parts CCP1, CCP2 and CCP3. The light controlling parts CCP1, CCP2 and CCP3 may be separated from one another.

Referring to FIG. 7, a partition pattern BMP may be provided between the separated light controlling parts CCP1, CCP2 and CCP3, but one or more embodiments of the present disclosure is not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 for converting first color light provided from the light emitting device ED into second color light, a second light controlling part CCP2 including a second quantum dot QD2 for converting first color light into third color light, and a third light controlling part CCP3 for transmitting first color light.

In one or more embodiments, the first light controlling part CCP1 may provide red light which is the second color light, and the second light controlling part CCP2 may provide green light which is the third color light. The third color controlling part CCP3 may transmit and provide blue light which is the first color light provided from the light emitting device ED. For example, the first quantum dot QD1 may be a red quantum dot, and the second quantum dot QD2 may be a green quantum dot. For the quantum dots QD1 and QD2, the same contents as those described above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot but may include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP, respectively. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking or reducing the penetration of moisture and/or oxygen (hereinafter, will be referred to as “humidity/oxygen”). The barrier layer BFL1 may be provided on the light controlling parts CCP1, CCP2 and CCP3 to block or reduce the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be provided between the light controlling parts CCP1, CCP2 and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride, and/or a metal thin film securing light transmittance. In some embodiments, the barrier layers BFL1 and BFL2 may further include an organic layer. The barrier layers BFL1 and BFL2 may be composed of a single layer of multiple layers.

In the display apparatus DD of one or more embodiments, the color filter layer CFL may be provided on the light controlling layer CCL. For example, the color filter layer CFL may be provided directly on the light controlling layer CCL. In this case, the barrier layer BFL2 may not be provided.

The color filter layer CFL may include a light blocking part BM and filters CF1, CF2 and CF3. The color filter layer CFL may include a first filter CF1 for transmitting second color light, a second filter CF2 for transmitting third color light, and a third filter CF3 for transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. However, one or more embodiments of the present disclosure is not limited thereto, and the third filter CF3 may not include (e.g., may exclude) the pigment or dye. The third filter CF3 may include a polymer photosensitive resin and not include a pigment or dye. The third filter CF3 may be transparent. The third filter CF3 may be formed utilizing a transparent photosensitive resin.

In some embodiments, in one or more embodiments, the first filter CF1 and the second filter CF2 may be yellow filters. The first filter CF1 and the second filter CF2 may be provided in one body without distinction (e.g., integrally).

The light blocking part may be a black matrix. The light blocking part may be formed by including an organic light blocking material or an inorganic light blocking material including a black pigment and/or black dye. The light blocking part may prevent or reduce light leakage phenomenon and divide the boundaries among adjacent filters CF1, CF2 and CF3. In some embodiments, in one or more embodiments, the light blocking part may be formed as a blue filter.

Each of the first to third filters CF1, CF2 and CF3 may be provided corresponding to each of a red luminous area PXA-R, green luminous area PXA-G, and blue luminous area PXA-B, respectively.

On the color filter layer CFL, a base substrate BL may be provided. The base substrate BL may be a member providing a base surface on which the color filter layer CFL, the light controlling layer CCL, etc. are provided. The base substrate BL may be a glass substrate, a metal substrate, a plastic substrate, etc. However, one or more embodiments of the present disclosure is not limited thereto, and the base substrate BL may be an inorganic layer, an organic layer or a composite material layer. In some embodiments, the base substrate BL may not be provided.

FIG. 8 is a cross-sectional view showing a portion of the display apparatus according to one or more embodiments. In a display apparatus DD-TD of one or more embodiments, the light emitting device ED-BT may include multiple light emitting structures OL-B1, OL-B2 and OL-B3. The light emitting device ED-BT may include oppositely provided first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 7), and a hole transport region HTR and an electron transport region ETR, provided with the emission layer EML (FIG. 7) therebetween.

For example, the light emitting device ED-BT included in the display apparatus DD-TD of one or more embodiments may be a light emitting device of a tandem structure including multiple emission layers.

In one or more embodiments shown in FIG. 8, light emitted from the light emitting structures OL-B1, OL-B2 and OL-B3 may be all blue light. However, one or more embodiments of the present disclosure is not limited thereto, and the wavelength regions of light emitted from the light emitting structures OL-B1, OL-B2 and OL-B3 may be different from each other. For example, the light emitting device ED-BT including the multiple light emitting structures OL-B1, OL-B2 and OL-B3 emitting light in different wavelength regions may be to emit white light.

Between neighboring light emitting structures OL-B1, OL-B2 and OL-B3, charge generating layers CGL1 and CGL2 may be provided. The charge generating layers CGL1 and CGL2 may include a p-type charge generating layer and/or an n-type charge generating layer.

In at least one selected from among the light emitting structures OL-B1, OL-B2 and OL-B3 included in the display apparatus DD-TD, the nitrogen-containing compound of one or more embodiments may be included. For example, at least one selected from among multiple emission layers included in the light emitting device ED-BT may include the nitrogen-containing compound of one or more embodiments.

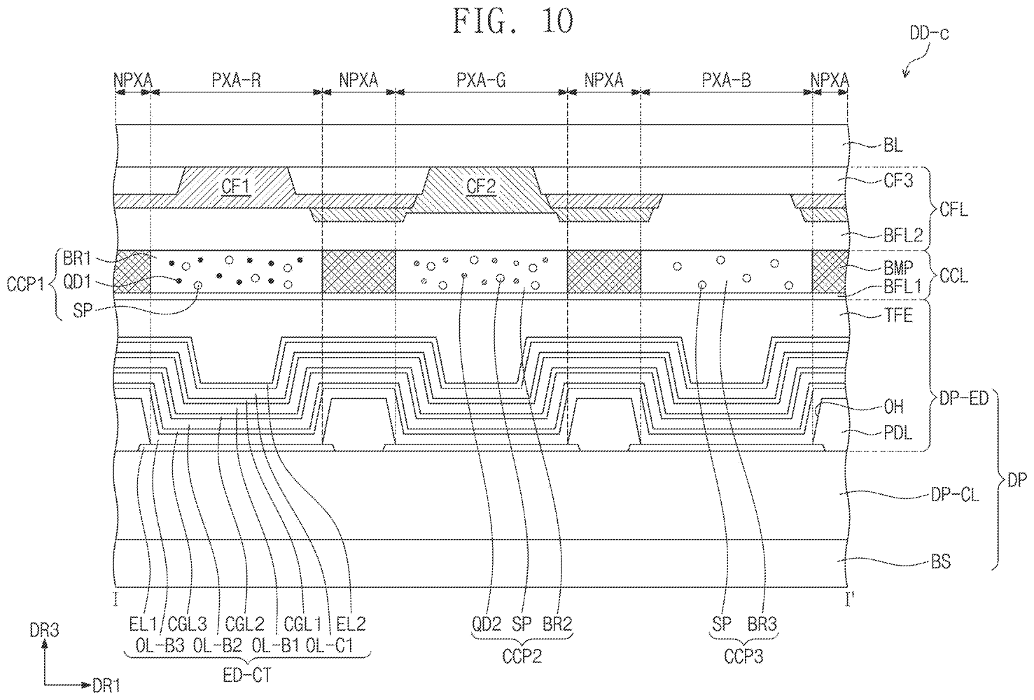

FIG. 9 is a cross-sectional view showing a display apparatus according to one or more embodiments of the present disclosure. FIG. 10 is a cross-sectional view showing a display apparatus according to one or more embodiments of the present disclosure.

Referring to FIG. 9, a display apparatus DD-b according to one or more embodiments may include light emitting devices ED-1, ED-2 and ED-3, formed by stacking two emission layers. Compared to the display apparatus DD of one or more embodiments, shown in FIG. 2, one or more embodiments shown in FIG. 9 is different in that first to third light emitting devices ED-1, ED-2 and ED-3 include two emission layers stacked in a thickness direction, each. In the first to third light emitting devices ED-1, ED-2 and ED-3, two emission layers may be to emit light in substantially the same wavelength region.

The first light emitting device ED-1 may include a first red emission layer EML-R₁ and a second red emission layer EML-R₂. The second light emitting device ED-2 may include a first green emission layer EML-G1 and a second green emission layer EML-G2. The third light emitting device ED-3 may include a first blue emission layer EML-B1 and a second blue emission layer EML-B2. Between the first red emission layer EML-R₁ and the second red emission layer EML-R₂, between the first green emission layer EML-G1 and the second green emission layer EML-G2, and between the first blue emission layer EML-B1 and the second blue emission layer EML-B2, an emission auxiliary part OG may be provided.

The emission auxiliary part OG may include a single layer or a multilayer. The emission auxiliary part OG may include a charge generating layer. For example, the emission auxiliary part OG may include an electron transport region, a charge generating layer, and a hole transport region stacked in order. The emission auxiliary part OG may be provided as a common layer in all of the first to third light emitting devices ED-1, ED-2 and ED-3. However, one or more embodiments of the present disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be provided between the electron transport region ETR and the emission auxiliary part OG. The second red emission layer EML-R2, the second green emission layer EML-G2 and the second blue emission layer EML-B2 may be provided between the emission auxiliary part OG and the hole transport region HTR.

For example, the first light emitting device ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting device ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting device ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be provided on a display element layer DP-ED. The optical auxiliary layer PL may include a polarization layer. The optical auxiliary layer PL may be provided on a display panel DP and may control reflected light at the display panel DP by external light. In some embodiments, the optical auxiliary layer PL may not be provided in the display apparatus according to one or more embodiments.

At least one emission layer included in the display apparatus DD-b of one or more embodiments, shown in FIG. 9, may include the nitrogen-containing compound of one or more embodiments. For example, in one or more embodiments, at least one selected from among the first blue emission layer EML-B1 and the second blue emission layer EML-B2 may include the nitrogen-containing compound of one or more embodiments.

Different from FIG. 8 and FIG. 9, a display apparatus DD-c in FIG. 10 is shown to include four light emitting structures OL-B1, OL-B2, OL-B3 and OL-C1. A light emitting device ED-CT may include oppositely provided first electrode EL1 and second electrode EL2, and first to fourth light emitting structures OL-B1, OL-B2, OL-B3 and OL-C1 stacked in order in a thickness direction between the first electrode EL1 and the second electrode EL2. Between the first to fourth light emitting structures OL-B1, OL-B2, OL-B3 and OL-C1, charge generating layers CGL1, CGL2 and CGL3 may be provided. Selected from among the four light emitting structures, the first to third light emitting structures OL-B1, OL-B2 and OL-B3 may be to emit blue light, and the fourth light emitting structure OL-C1 may be to emit green light. However, one or more embodiments of the present disclosure is not limited thereto, and the first to fourth light emitting structures OL-B1, OL-B2, OL-B3 and OL-C1 may be to emit different wavelengths of light.

The charge generating layers CGL1, CGL2 and CGL3 provided between the neighboring light emitting structures OL-B1, OL-B2, OL-B3 and OL-C1 may include a p-type charge generating layer and/or an n-type charge generating layer.

In at least one selected from among the light emitting structures OL-B1, OL-B2, OL-B3 and OL-C1 included in the display apparatus DD-c, the nitrogen-containing compound of one or more embodiments may be included. For example, in one or more embodiments, at least one selected from among the first to third light emitting structures OL-B1, OL-B2 and OL-B3 may include the nitrogen-containing compound of one or more embodiments.

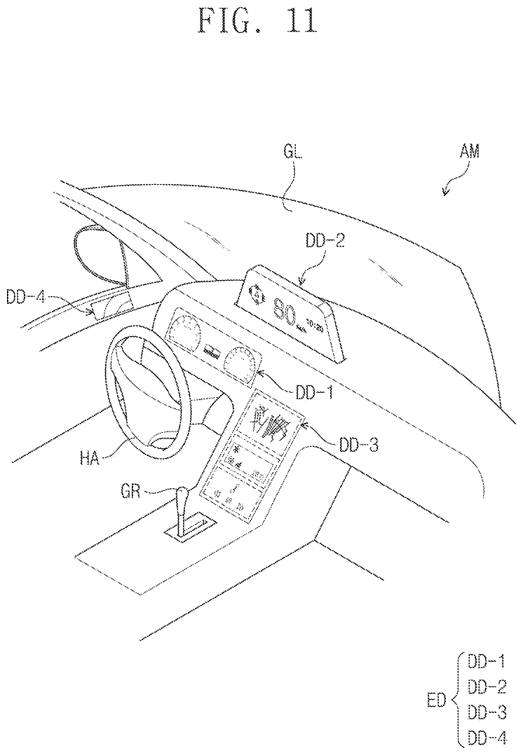

FIG. 11 is a perspective view schematically showing an electronic apparatus including the display apparatus according to one or more embodiments. In FIG. 11, an electronic apparatus including display apparatuses for automobiles is shown as an illustration.

Referring to FIG. 11, an electronic apparatus ED of one or more embodiments may include display apparatuses DD-1, DD-2, DD-3 and DD-4 for an automobile AM. In FIG. 11, first to fourth display apparatuses DD-1, DD-2, DD-3 and DD-4 are shown as display apparatuses for an automobile AM, provided in the automobile AM. In FIG. 11, an automobile is shown as an illustration, but the first to fourth display apparatuses DD-1, DD-2, DD-3 and DD-4 may be provided in one or more suitable transport means such as bicycles, motorcycles, trains, ships and/or airplanes. At least one selected from among the first to fourth display apparatuses DD-1, DD-2, DD-3 and DD-4 may include the configuration of the display apparatuses DD, DD-a, DD-TD, DD-b and DD-c, explained above referring to FIG. 1, FIG. 2, and FIG. 7 to FIG. 10.

In one or more embodiments, at least one selected from among the first to fourth display apparatuses DD-1, DD-2, DD-3 and DD-4 may include the light emitting device ED explained with reference to FIG. 3 to FIG. 6. The first to fourth display apparatuses DD-1, DD-2, DD-3 and DD-4 may each independently include multiple light emitting devices ED, and each of the light emitting devices ED may include a first electrode EL1, a hole transport region HTL provided on the first electrode EL1, an emission layer EML provided on the hole transport region HTL, an electron transport region ETL provided on the emission layer EML and a second electrode EL2 provided on the electron transport region ETL. In some embodiments, the emission layer EML may include the nitrogen-containing compound of one or more embodiments, represented by Formula 1. Accordingly, the electronic apparatus ED of one or more embodiments may show improved quality of images.

Referring to FIG. 11, the automobile AM may include a steering wheel HA for the operation of the automobile AM and a gear GR, and a front window GL may be provided to face a driver.

A first display apparatus DD-1 may be provided in a first region overlapping with the steering wheel HA. For example, the first display apparatus DD-1 may be a digital cluster displaying the first information of the automobile AM. The first information may include a graduation (graduated) showing the number of revolutions of an engine (i.e., revolutions per minute (RPM)), and images showing a fuel state. First graduation and second graduation may be represented by digital images.

A second display apparatus DD-2 may be provided in a second region facing a driver's seat and overlapping with the front window GL. The driver's seat may be a seat where the steering wheel HA is provided. For example, the second display apparatus DD-2 may be a head up display (HUD) showing the second information of the automobile AM. The second display apparatus DD-2 may be optically clear. The second information may include digital numbers DN showing the running speed of the automobile AM and may further include information including the current time.

A third display apparatus DD-3 may be provided in a third region adjacent to the gear GR. For example, the third display apparatus DD-3 may be a center information display (CID) for an automobile, provided between a driver's seat and a passenger seat and showing third information. The passenger seat may be a seat separated from the driver's seat with the gear GR therebetween. The third information may include information on road conditions (for example, navigation information), on playing music and/or radio, on playing a dynamic image, on the temperature in the automobile AM, and/or the like.

A fourth display apparatus DD-4 may be provided in a fourth region separated from the steering wheel HA and the gear GR and adjacent to the side of the automobile AM. For example, the fourth display apparatus DD-4 may be a digital wing mirror displaying fourth information. The fourth display apparatus DD-4 may display the external image of the automobile AM, taken by a camera module CM provided at the outside of the automobile AM. The fourth information may include the external image of the automobile AM.

The above-described first to fourth information is for illustration, and the first to fourth display apparatuses DD-1, DD-2, DD-3 and DD-4 may further display information on the inside and outside of the automobile. The first to fourth information may include different information from each other. However, one or more embodiments of the present disclosure is not limited thereto, and a portion of the first to fourth information may include the same information.

Hereinafter, referring to embodiments and comparative embodiments, the nitrogen-containing compound according to one or more embodiments and the light emitting device according to one or more embodiments of the present disclosure will be explained in more detail. However, the embodiments are only illustrations to assist the understanding of the present disclosure, and the scope of the present disclosure is not limited thereto.

### EXAMPLES

1. Synthesis of Nitrogen-Containing Compound

First, the synthetic method of the nitrogen-containing compound according to one or more embodiments will be explained in particular referring to the synthetic methods of Compounds 1, 11, 182, 213, 415, 435, 545 and 618. In some embodiments, the synthetic methods of the nitrogen-containing compounds explained hereinafter are only embodiments, and the synthetic method of the nitrogen-containing compound according to one or more embodiments of the present disclosure is not limited to the embodiments.

(1) Synthesis of Compound 1

Nitrogen-containing Compound 1 according to one or more embodiments may be synthesized by, for example, the reactions below.

(Synthesis of Intermediate 1-1)

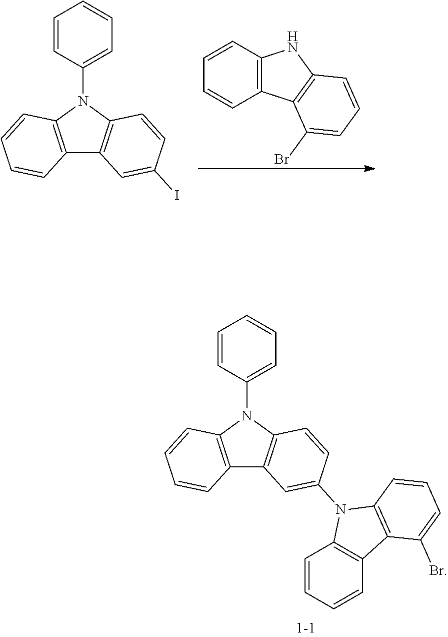 [SMILES: Ic1ccc2c(c1)c1ccccc1n2-c1ccccc1 ; Brc1cccc2[nH]c3ccccc3c12 ; Brc1cccc2c1c1ccccc1n2-c1ccc2c(c1)c1ccccc1n2-c1ccccc1]

3-Iodo-9-phenyl-9H-carbazole (CAS No.=502161-03-7) and 4-bromo-9H-carbazole (CAS No.=3652-89-9) were reacted under a Cu catalyst to obtain Intermediate 1-1. The M+1 peak value of Intermediate 1-1 was found utilizing liquid chromatography-mass spectrometry (LC-MS).

C₃₀H₁₉BrN₂: M+1 487.07

(Synthesis of Compound 1)

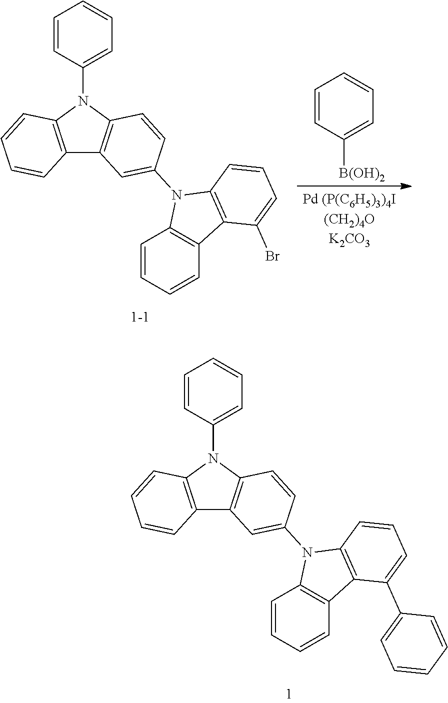 [SMILES: Brc1cccc2c1c1ccccc1n2-c1ccc2c(c1)c1ccccc1n2-c1ccccc1 ; OB(O)c1ccccc1 ; c1ccc(-c2cccc3c2c2ccccc2n3-c2ccc3c(c2)c2ccccc2n3-c2ccccc2)cc1]

5 g of Intermediate 1-1, 1.4 g of phenylboronic acid (CAS No=98-80-6), 0.6 g of tetrakis(triphenylphosphine)palladium(0) and 3.5 g of potassium carbonate were put in a reaction vessel and dissolved in 50 mL of tetrahydrofuran and 12 mL of distilled water, followed by refluxing for about 24 hours. After finishing the reaction, the reaction solution was extracted with ethyl acetate, and an organic layer collected was dried over magnesium sulfate. The residue obtained by evaporating the solvent was separated and purified by silica gel column chromatography to obtain 3.8 g (yield: 78%) of Compound 1. Compound 1 was identified by LC-MS and ¹H-NMR.

(2) Synthesis of Compound 11

Nitrogen-containing Compound 11 according to one or more embodiments may be synthesized by, for example, the reactions below.

(Synthesis of Intermediate 11-1)

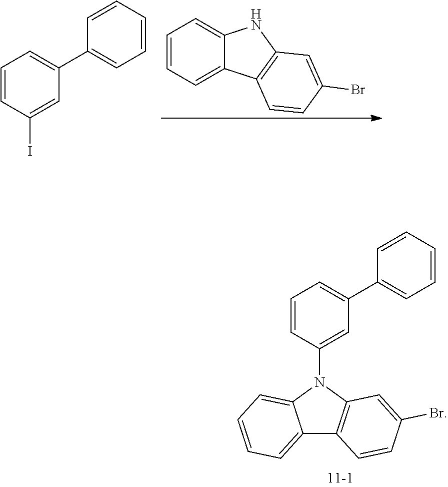

3-Iodo-biphenyl (CAS No.=20442-79-9) and 2-bromocarbazole (CAS No.=3652-90-2) were reacted under a Cu catalyst to obtain Intermediate 11-1. The M+1 peak value of Intermediate 11-1 was found utilizing liquid chromatography-mass spectrometry (LC-MS).

C₂₄H₁₆BrN: M+1 398.06

(Synthesis of Intermediate 11-2)

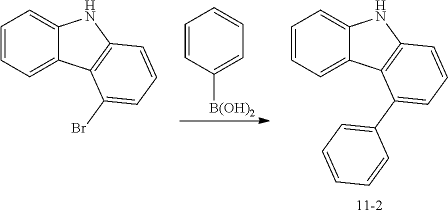

4-Bromo-9H-carbazole (CAS No.=3652-89-9) and phenylboronic acid (CAS No.=98-80-6) were reacted under a Pd catalyst to obtain Intermediate 11-2. The M+1 peak value of Intermediate 11-2 was found utilizing liquid chromatography-mass spectrometry (LC-MS).

C₁₈H₁₃N: M+1 244.13

(Synthesis of Compound 11)

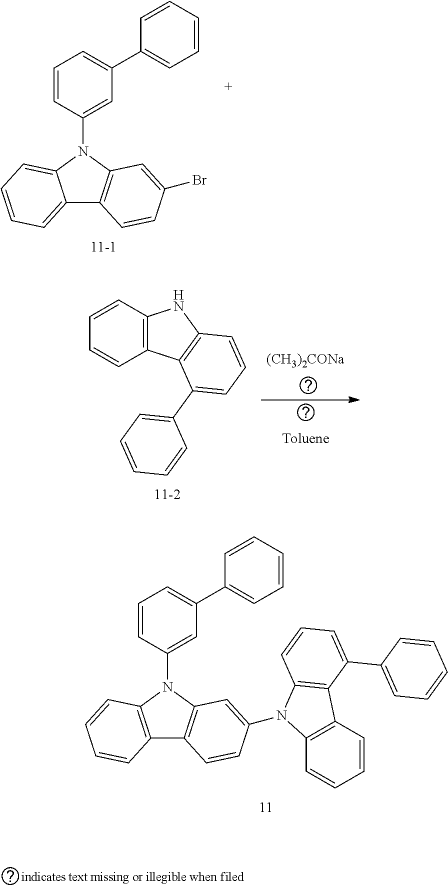

4 g of Intermediate 11-1, 2.5 g of Intermediate 11-2, 1.5 g of sodium butoxide, 0.37 g of tris(dibenzylideneacetone)dipalladium(0), 0.35 mL of tri-tert-butylphosphine and 50 mL of toluene were put in a reaction vessel and refluxed for about 24 hours. After finishing the reaction, the reaction solution was extracted with ethyl acetate, and an organic layer collected was dried over magnesium sulfate. The residue obtained by evaporating the solvent was separated and purified by silica gel column chromatography to obtain 4.7 g (yield: 84%) of Compound 11. Compound 11 was identified by LC-MS and ¹H-NMR.

(3) Synthesis of Compound 182

Nitrogen-containing Compound 182 according to one or more embodiments may be synthesized by, for example, the reactions below.

(Synthesis of Intermediate 182-1)

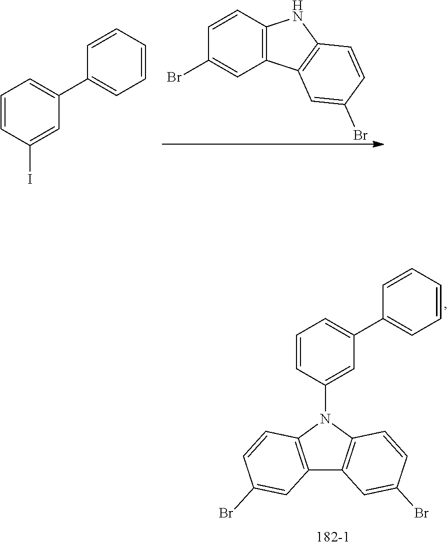

3-Iodo-biphenyl (CAS No.=20442-79-9) and 3,6-dibromocarbazole (CAS No.=6825-20-3) were reacted under a Cu catalyst to obtain Intermediate 182-1. The M+1 peak value of Intermediate 182-1 was found utilizing liquid chromatography-mass spectrometry (LC-MS).

C₂₄H₁₅Br₂N: M+1 475.96

(Synthesis of Intermediate 182-2)

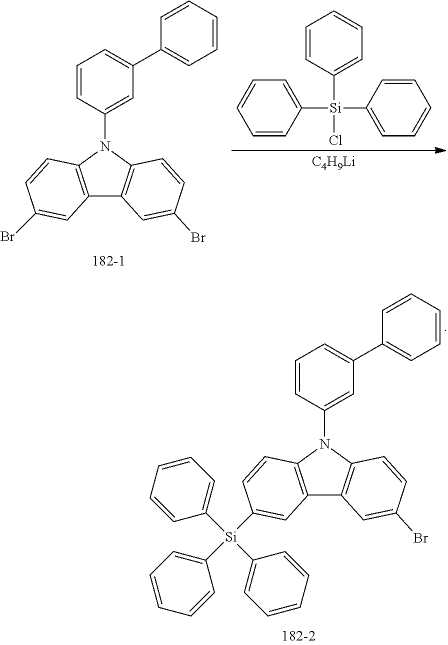

Intermediate 182-1 was reacted with n-BuLi and then, reacted with chlorotriphenylsilane (CAS No=76-86-8) to obtain Intermediate 182-2. The M+1 peak value of Intermediate 182-2 was found utilizing liquid chromatography-mass spectrometry (LC-MS).

C₄₂H₃₀BrNSi: M+1 656.14

(Synthesis of Compound 182)

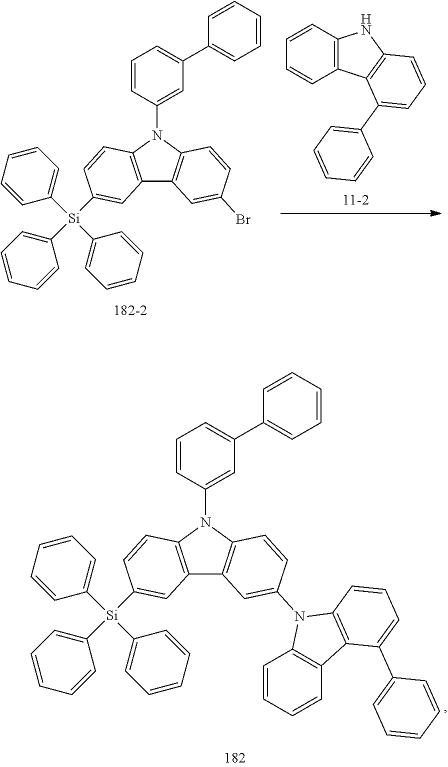

Substantially the same method as the synthetic method of Compound 11 was performed except for utilizing Intermediate 182-2 instead of Intermediate 11-2. 4.4 g (yield: 70%) of Compound 182 was obtained. Compound 182 was identified by LC-MS and ¹H-NMR.

(4) Synthesis of Compound 213

Nitrogen-containing Compound 213 according to one or more embodiments may be synthesized by, for example, the reactions below.

(Synthesis of Intermediate 213-1)

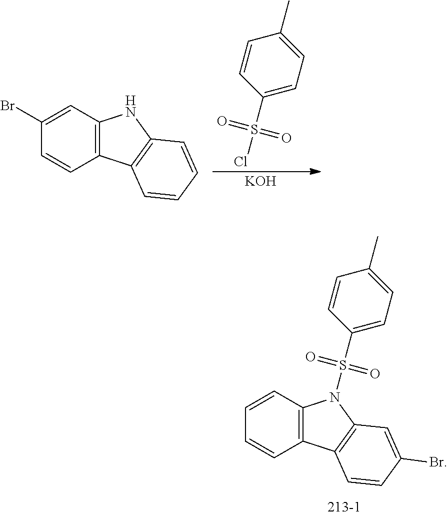

2-Bromo-9H-carbazole (CAS No.=3652-90-2), potassium hydroxide and 4-toluenesulfonyl chloride (CAS No.=98-59-9) were reacted to obtain Intermediate 213-1. The M+1 peak value of Intermediate 213-1 was found utilizing liquid chromatography-mass spectrometry (LC-MS).

C₁₉H₁₄BrNO₂S: M+1 399.99

(Synthesis of Intermediate 213-2)

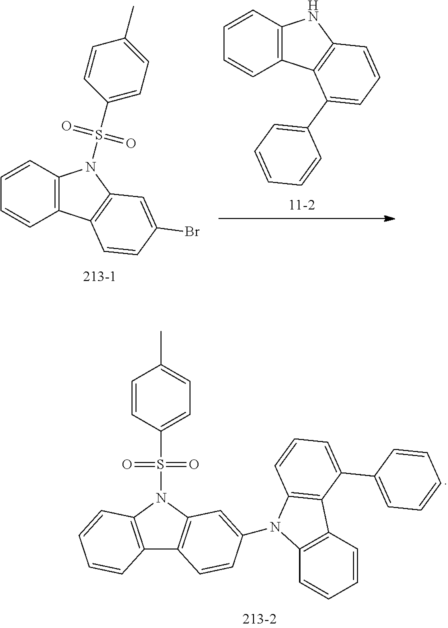 [SMILES: Cc1ccc(S(=O)(=O)n2c3ccccc3c3ccc(Br)cc32)cc1 ; c1ccc(-c2cccc3[nH]c4ccccc4c23)cc1 ; Cc1ccc(S(=O)(=O)n2c3ccccc3c3ccc(-n4c5ccccc5c5c(-c6ccccc6)cccc54)cc32)cc1]

Intermediate 213-1 and Intermediate 11-2 were reacted under a Cu catalyst to obtain Intermediate 213-2. The M+1 peak value of Intermediate 213-2 was found utilizing liquid chromatography-mass spectrometry (LC-MS).

C₃₇H₂₆N₂O₂S: M+1 563.15

(Synthesis of Intermediate 213-3)

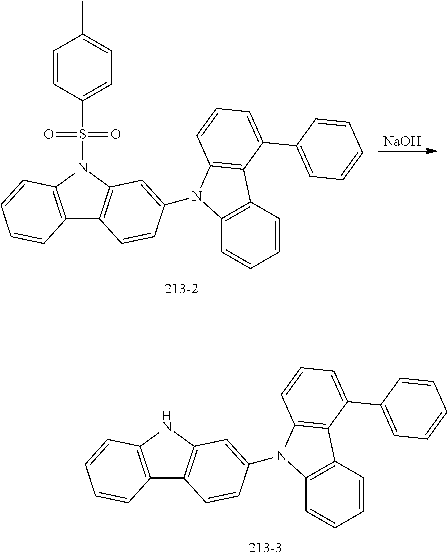

Intermediate 213-2 and sodium hydroxide were reacted to obtain Intermediate 213-3. The M+1 peak value of Intermediate 213-3 was found utilizing liquid chromatography-mass spectrometry (LC-MS).

C₃₀H₂₀N₂: M+1 409.18

(Synthesis of Compound 213)

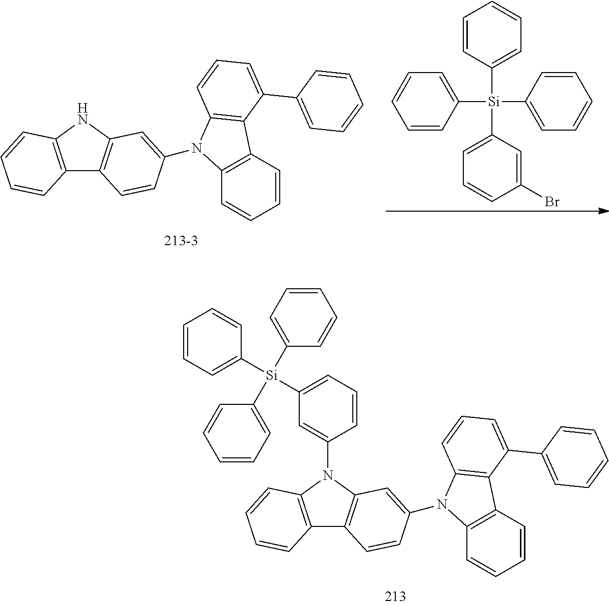

Substantially the same method as the synthetic method of Compound 11 was performed except for utilizing (3-bromophenyl)triphenylsilane (CAS No.=185626-73-7) and Intermediate 213-3 instead of Intermediate 11-1 and Intermediate 11-2. 7.5 g (yield: 70%) of Compound 213 was obtained. Compound 213 was identified by LC-MS and ¹H-NMR.

(5) Synthesis of Compound 415

Nitrogen-containing Compound 415 according to one or more embodiments may be synthesized by, for example, the reactions below.

(Synthesis of Intermediate 415-1)

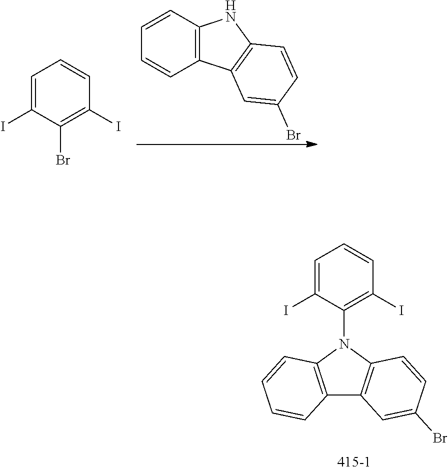 [SMILES: Brc1c(I)cccc1I ; Brc1ccc2[nH]c3ccccc3c2c1 ; Brc1ccc2c(c1)c1ccccc1n2-c1c(I)cccc1I]

2-Fluoro-1,3-diiodobenzene (CAS No.=1435-54-7) and 3-bromo-9H-carbazole (CAS No.=1592-95-6) were reacted to obtain Intermediate 415-1. The M+1 peak value of Intermediate 415-1 was found utilizing liquid chromatography-mass spectrometry (LC-MS).

C₁₈H₁₀Br₁₂N: M+1 593.83

(Synthesis of Intermediate 415-2)

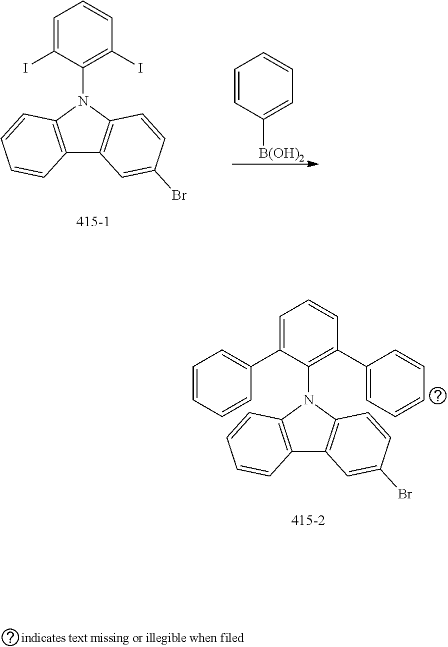 [SMILES: Brc1ccc2c(c1)c1ccccc1n2-c1c(I)cccc1I ; OB(O)c1ccccc1 ; Brc1ccc2c(c1)c1ccccc1n2-c1c(-c2ccccc2)cccc1-c1ccccc1]

Intermediate 415-1 and phenylboronic acid (CAS No.=98-80-6) were reacted under a Pd catalyst to obtain Intermediate 415-2. The M+1 peak value of Intermediate 415-2 was found utilizing liquid chromatography-mass spectrometry (LC-MS).

C₃₀H₂₀BrN: M+1 474.08

(Synthesis of Intermediate 415-3)

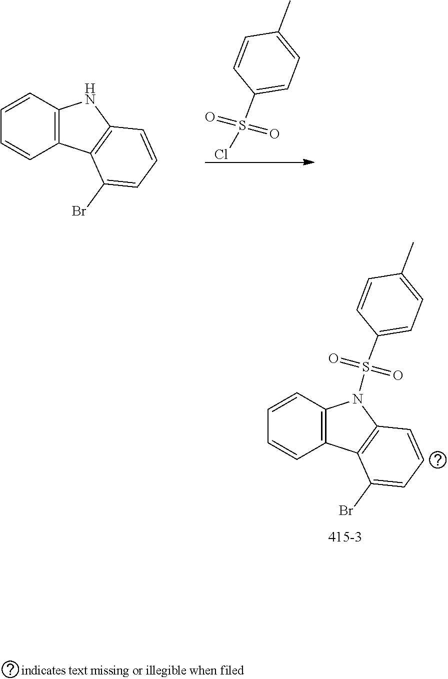 [SMILES: Brc1cccc2[nH]c3ccccc3c12 ; Cc1ccc(S(=O)(=O)Cl)cc1 ; Cc1ccc(S(=O)(=O)n2c3ccccc3c3c(Br)cccc32)cc1]

Substantially the same method as the synthetic method of Intermediate 213-1 was performed except for utilizing 4-bromo-9H-carbazole (CAS No.=3652-89-9) instead of 2-bromo-9H-carbazole (CAS No.=3652-90-2) to obtain Intermediate 415-3. The M+1 peak value of Intermediate 415-3 was found utilizing liquid chromatography-mass spectrometry (LC-MS).

C₁₉H₁₄BrNO₂S: M+1 399.98

(Synthesis of Intermediate 415-4)

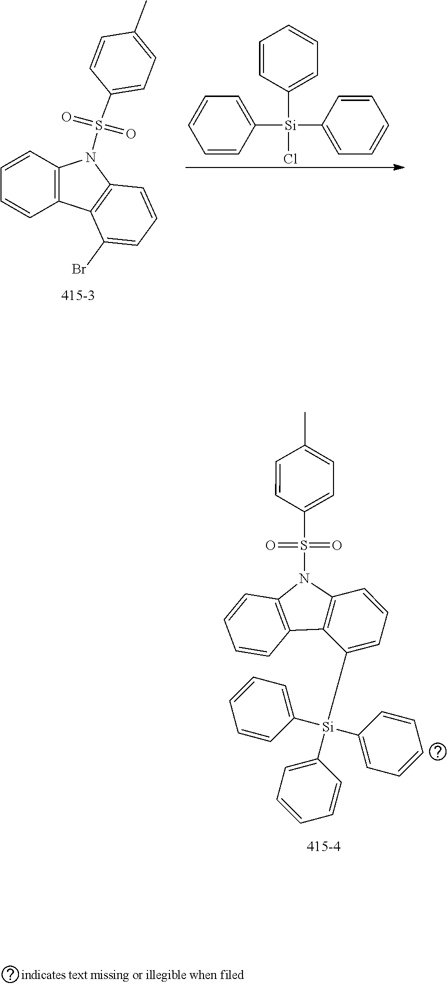 [SMILES: Cc1ccc(S(=O)(=O)n2c3ccccc3c3c(Br)cccc32)cc1 ; Cl[Si](c1ccccc1)(c1ccccc1)c1ccccc1 ; Cc1ccc(S(=O)(=O)n2c3ccccc3c3c([Si](c4ccccc4)(c4ccccc4)c4ccccc4)cccc32)cc1]

Substantially the same method as the synthetic method of Intermediate 182-2 was performed except for utilizing Intermediate 415-3 instead of Intermediate 182-1 to obtain Intermediate 415-4. The M+1 peak value of Intermediate 415-4 was found utilizing liquid chromatography-mass spectrometry (LC-MS).

C₃₇H₂₉NO₂SSi: M+1 580.16

(Synthesis of Intermediate 415-5)

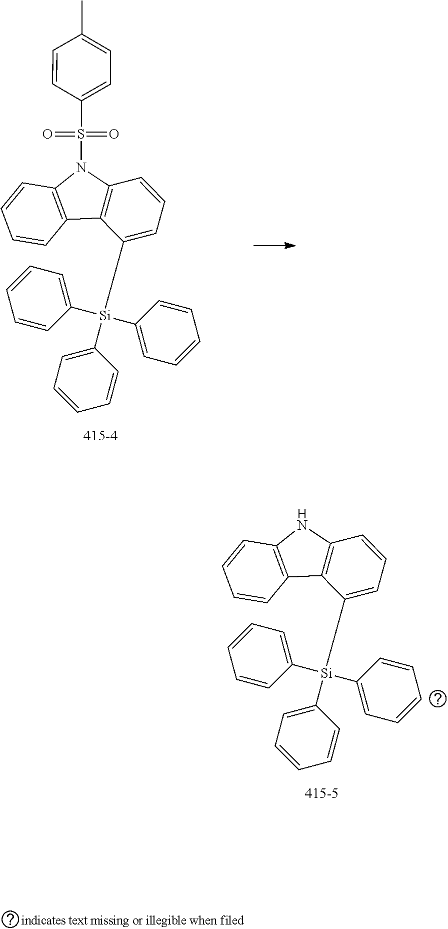 [SMILES: Cc1ccc(S(=O)(=O)n2c3ccccc3c3c([Si](c4ccccc4)(c4ccccc4)c4ccccc4)cccc32)cc1 ; c1ccc([Si](c2ccccc2)(c2ccccc2)c2cccc3[nH]c4ccccc4c23)cc1]

Substantially the same method as the synthetic method of Intermediate 213-3 was performed except for utilizing Intermediate 415-4 instead of Intermediate 213-2 to obtain Intermediate 415-5. The M+1 peak value of Intermediate 415-5 was found utilizing liquid chromatography-mass spectrometry (LC-MS).

C₃₀H₂₃NSi: M+1 426.18

(Synthesis of Compound 415)

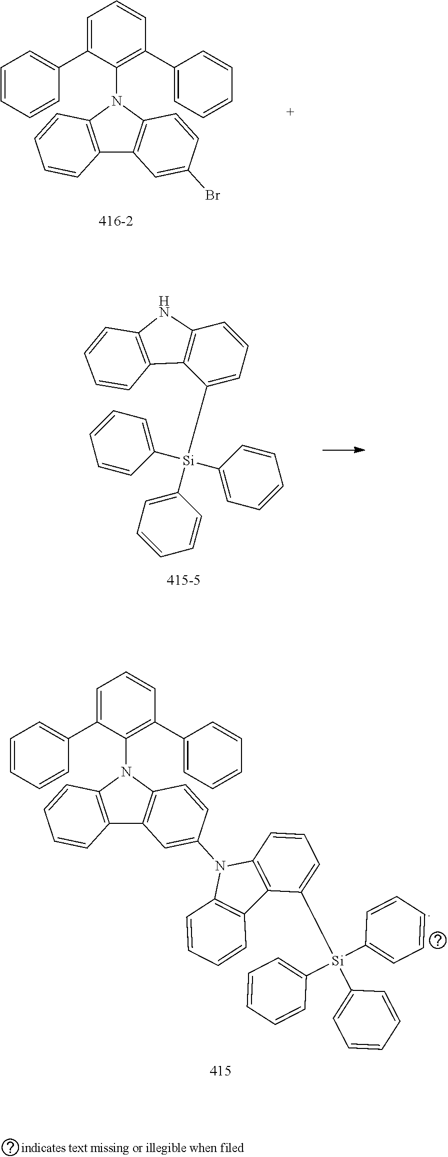 [SMILES: Brc1ccc2c(c1)c1ccccc1n2-c1c(-c2ccccc2)cccc1-c1ccccc1 ; c1ccc([Si](c2ccccc2)(c2ccccc2)c2cccc3[nH]c4ccccc4c23)cc1 ; c1ccc(-c2cccc(-c3ccccc3)c2-n2c3ccccc3c3cc(-n4c5ccccc5c5c([Si](c6ccccc6)(c6ccccc6)c6ccccc6)cccc54)ccc32)cc1]

Substantially the same method as the synthetic method of Compound 11 was performed except for utilizing Intermediate 415-2 and Intermediate 415-5 instead of Intermediate 11-1 and Intermediate 11-2. 5.9 g (yield: 68%) of Compound 415 was obtained. Compound 415 was identified by LC-MS and ¹H-NMR.

(6) Synthesis of Compound 435

Nitrogen-containing Compound 435 according to one or more embodiments may be synthesized by, for example, the reactions below.

(Synthesis of Intermediate 435-1)

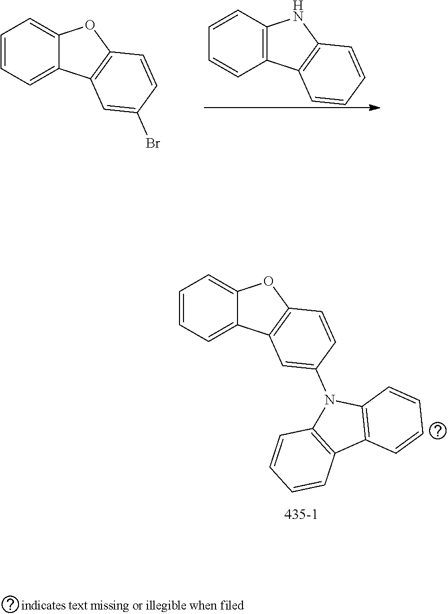 [SMILES: Brc1ccc2oc3ccccc3c2c1 ; c1ccc2c(c1)[nH]c1ccccc12 ; c1ccc2c(c1)oc1ccc(-n3c4ccccc4c4ccccc43)cc12]

2-Bromodibenzofuran (CAS No.=86-76-0) and 9H-carbazole (CAS No.=86-74-8) were reacted under a Pd catalyst to obtain Intermediate 435-1. The M+1 peak value of Intermediate 435-1 was found utilizing liquid chromatography-mass spectrometry (LC-MS).

C₂₄H₁₅NO: M+1 334.11

(Synthesis of Intermediate 435-2)

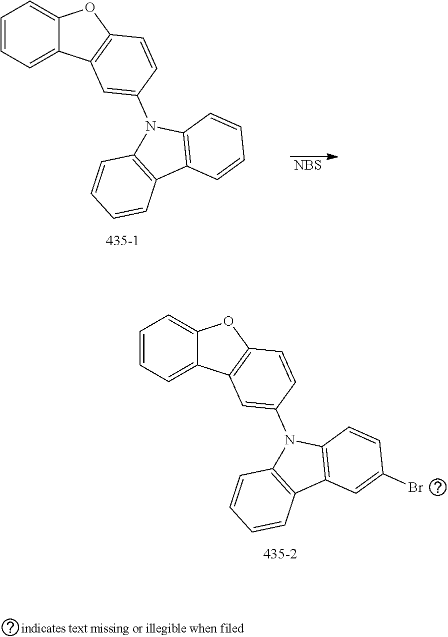

Intermediate 435-1 and N-bromosuccinimide were reacted to obtain Intermediate 435-2. The M+1 peak value of Intermediate 435-2 was found utilizing liquid chromatography-mass spectrometry (LC-MS).

C₂₄H₁₄BrNO: M+1 412.03

(Synthesis of Intermediate 435-3)

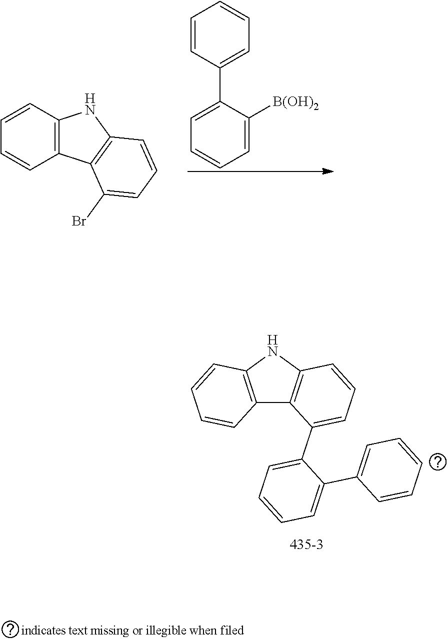

4-Bromo-9H-carbazole (CAS No.=3652-89-2) and 2-biphenylboronic acid (CAS No.=4688-76-0) were reacted under a Pd catalyst to obtain Intermediate 435-3. The M+1 peak value of Intermediate 435-3 was found utilizing liquid chromatography-mass spectrometry (LC-MS).

C₂₄H₁₇N: M+1 420.15

(Synthesis of Compound 435)

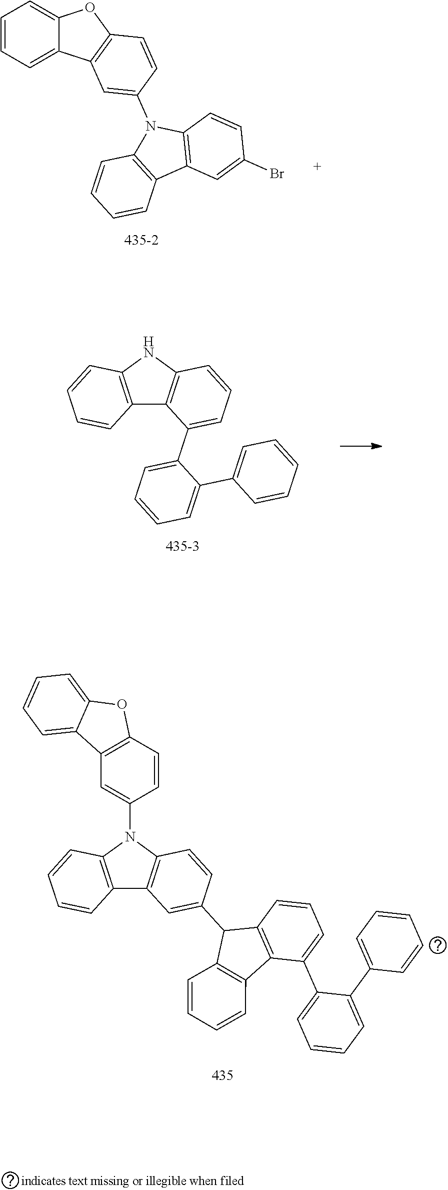 [SMILES: Brc1ccc2c(c1)c1ccccc1n2-c1ccc2oc3ccccc3c2c1 ; c1ccc(-c2ccccc2-c2cccc3[nH]c4ccccc4c23)cc1 ; c1ccc(-c2ccccc2-c2cccc3c2-c2ccccc2C3c2ccc3c(c2)c2ccccc2n3-c2ccc3oc4ccccc4c3c2)cc1]

Substantially the same method as the synthetic method of Compound 11 was performed except for utilizing Intermediate 435-2 and Intermediate 435-3 instead of Intermediate 11-1 and Intermediate 11-2. 4.8 g (yield: 75%) of Compound 435 was obtained. Compound 435 was identified by LC-MS and ¹H-NMR.

(7) Synthesis of Compound 545

Nitrogen-containing Compound 545 according to one or more embodiments may be synthesized by, for example, the reactions below.

(Synthesis of Intermediate 545-1)

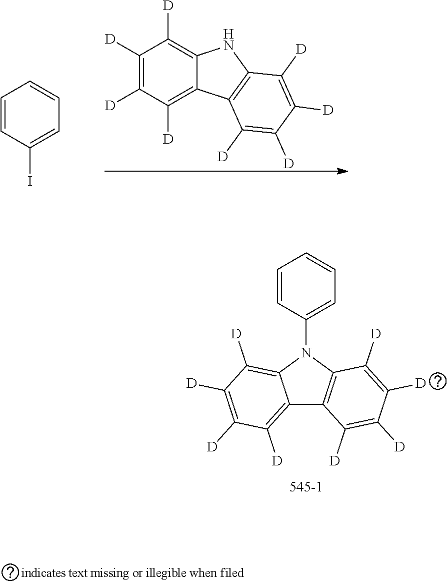 [SMILES: Ic1ccccc1 ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1ccccc1]

Iodobenzene (CAS No.=591-50-4) and 9H-carbazole-1,2,3,4,5,6,7,8-d8 (CAS No.=38537-24-5) were reacted under a Cu catalyst to obtain Intermediate 545-1. The M+1 peak value of Intermediate 545-1 was found utilizing liquid chromatography-mass spectrometry (LC-MS).

C₁₈H₅D₈N: M+1 252.15

(Synthesis of Intermediate 545-2)

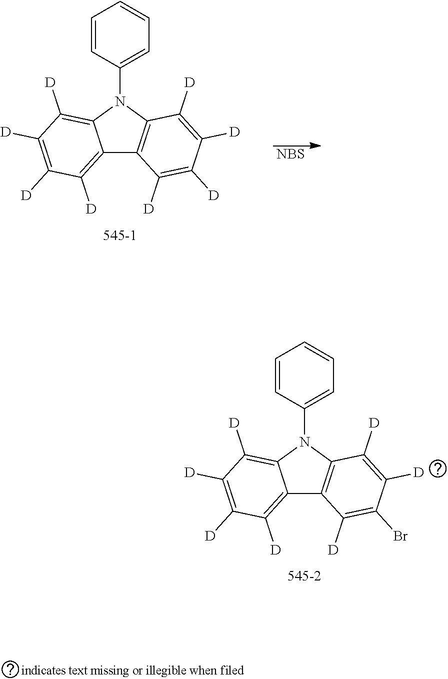

Intermediate 545-1 and N-bromosuccinimide were reacted to obtain Intermediate 545-2. The M+1 peak value of Intermediate 545-2 was found utilizing liquid chromatography-mass spectrometry (LC-MS).

C₁₈H₅D₇BrN: M+1 329.07

(Synthesis of Compound 545)

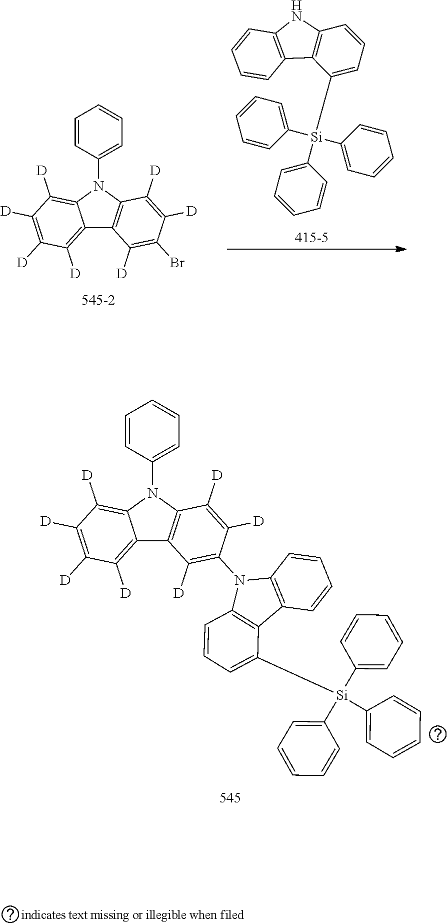 [SMILES: [2H]c1c([2H])c([2H])c2c(c1[2H])c1c([2H])c(Br)c([2H])c([2H])c1n2-c1ccccc1 ; c1ccc([Si](c2ccccc2)(c2ccccc2)c2cccc3[nH]c4ccccc4c23)cc1 ; [2H]c1c([2H])c([2H])c2c(c1[2H])c1c([2H])c(-n3c4ccccc4c4c([Si](c5ccccc5)(c5ccccc5)c5ccccc5)cccc43)c([2H])c([2H])c1n2-c1ccccc1]

Substantially the same method as the synthetic method of Compound 11 was performed except for utilizing Intermediate 545-2 and Intermediate 415-5 instead of Intermediate 11-1 and Intermediate 11-2. 4.9 g (yield: 80%) of Compound 545 was obtained. Compound 545 was identified by LC-MS and ¹H-NMR.

(8) Synthesis of Compound 618

Nitrogen-containing Compound 618 according to one or more embodiments may be synthesized by, for example, the reactions below.

(Synthesis of Intermediate 618-1)

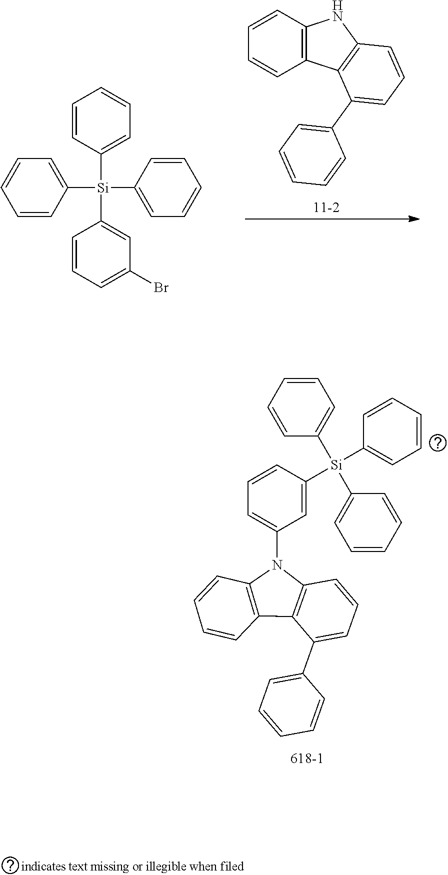 [SMILES: Brc1cccc([Si](c2ccccc2)(c2ccccc2)c2ccccc2)c1 ; c1ccc(-c2cccc3[nH]c4ccccc4c23)cc1 ; c1ccc(-c2cccc3c2c2ccccc2n3-c2cccc([Si](c3ccccc3)(c3ccccc3)c3ccccc3)c2)cc1]

(3-Bromophenyl)triphenylsilane (CAS No.=185626-73-7) and Intermediate 11-2 were reacted under a Pd catalyst to obtain Intermediate 618-1. The M+1 peak value of Intermediate 618-1 was found utilizing liquid chromatography-mass spectrometry (LC-MS).

C₄₂H₃₁NSi: M+1 578.23

(Synthesis of Intermediate 618-2)

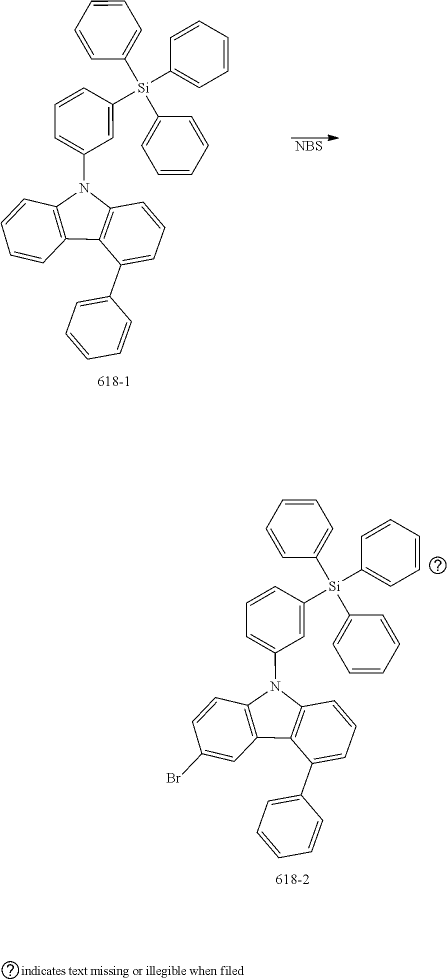 [SMILES: c1ccc(-c2cccc3c2c2ccccc2n3-c2cccc([Si](c3ccccc3)(c3ccccc3)c3ccccc3)c2)cc1 ; "NBS" ; Brc1ccc2c(c1)c1c(-c3ccccc3)cccc1n2-c1cccc([Si](c2ccccc2)(c2ccccc2)c2ccccc2)c1]

Intermediate 618-1 and N-bromosuccinimide were reacted to obtain Intermediate 618-2. The M+1 peak value of Intermediate 618-2 was found utilizing liquid chromatography-mass spectrometry (LC-MS).

C₄₂H₃₀BrNSi: M+1 656.14

(Synthesis of Compound 618)

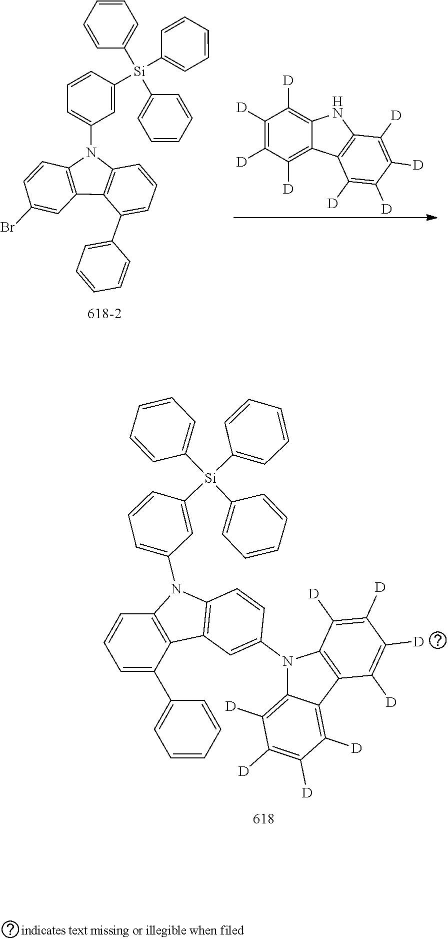 [SMILES: Brc1ccc2c(c1)c1c(-c3ccccc3)cccc1n2-c1cccc([Si](c2ccccc2)(c2ccccc2)c2ccccc2)c1 ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1ccc2c(c1)c1c(-c3ccccc3)cccc1n2-c1cccc([Si](c2ccccc2)(c2ccccc2)c2ccccc2)c1]

Substantially the same method as the synthetic method of Compound 11 was performed except for utilizing Intermediate 618-2 and 9H-carbazole-1,2,3,4,5,6,7,8-d8 (CAS No.=38537-24-5) instead of Intermediate 11-1 and Intermediate 11-2. 3.6 g (yield: 63%) of Compound 618 was obtained. Compound 618 was identified by LC-MS and ¹H-NMR.

The ¹H NMR and MS/FAB of the compounds synthesized in Synthetic Examples (1) to (8) are shown in Table 1. The synthetic methods of other compounds could be easily recognized by a person skilled in the art referring to the synthetic processes and raw materials above.

TABLE 1

| | | MS- | MS- |
|---|---|---|---|
| Compound | 1H NMR chemical shift | Cal | Meas. |
| 1 | 8.55 (d, 2H), 7.94 (d, 2H), 7.91 (d, 1H), | 484.19 | 485.21 |
| | 7.79-7.38 (m, 17H), 7.16 (t, 2H) | | |
| 11 | 8.55 (d, 2H), 8.26 (d, 1H), 8.21 (s, 1H), | 560.23 | 561.23 |
| | 7.94-7.91 (m, 3H), 7.78-7.37 (m, 19H), | | |
| | 7.16 (t, 2H) | | |
| 182 | 8.55 (d, 1H), 8.21 (s, 1H), 8.08 (d, 1H), | 818.31 | 819.33 |
| | 7.94-7.91 (m, 2H), 7.78-7.67 (m, 10H), | | |
| | 7.56-7.35 (m, 26H), 7.16 (t, 1H) | | |
| 213 | 8.55 (d, 2H), 8.26 (d, 1H), 7.94 (d, 2H), 7.91 | 742.28 | 743.31 |
| | (d, 1H), 7.79 (d, 2H), 7.68 (d, 1H), 7.66 (s, | | |
| | 1H), 7.56-7.30 (m, 26H), 7.16 (t, 2H) | | |
| 415 | 8.55 (d, 2H), 8.01 (d, 2H), 7.94 (d, 2H), | 818.31 | 819.32 |
| | 7.72-7.67 (m, 4H), 7.47-7.35 (m, 22H), | | |
| | 7.19-7.16 (m, 10H) | | |
| 435 | 8.55 (d, 2H), 7.98-7.94 (m, 6H), 7.68-7.31 | 650.24 | 651.20 |
| | (m, 20H), 7.16 (t, 2H) | | |
| 545 | 8.55 (d, 1H), 7.94 (d, 1H), 7.68-7.37 (m, | 673.29 | 674.31 |
| | 24H), 7.16 (t, 1H) | | |
| 618 | 8.08 (d, 1H), 7.91 (d, 1H), 7.79-7.66 (m, | 750.33 | 751.35 |
| | 6H), 7.60-7.56 (m, 3H), 7.46-7.38 (m, 19H) | | |

2. Manufacture and Evaluation of Light Emitting Device Including Nitrogen-Containing Compound

A light emitting device of one or more embodiments, including a nitrogen-containing compound of one or more embodiments in an emission layer was manufactured by a method below. Light emitting devices of Example 1 to Example 8 were manufactured utilizing the nitrogen-containing compound of Compounds 1, 11, 182, 213, 415, 435, 545 and 618, respectively. Comparative Example 1 to Comparative Example 4 correspond to light emitting devices manufactured utilizing Comparative Compound C1 to Comparative Compound C4 as the dopant materials of an emission layer, respectively.

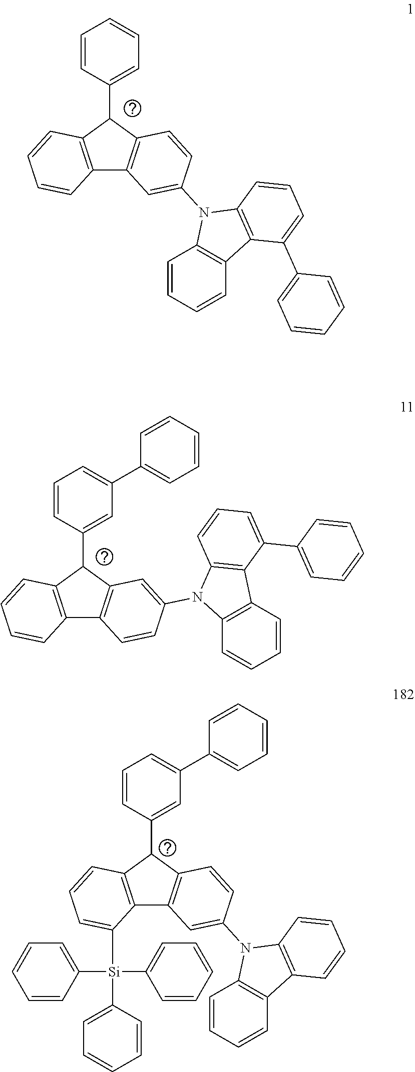

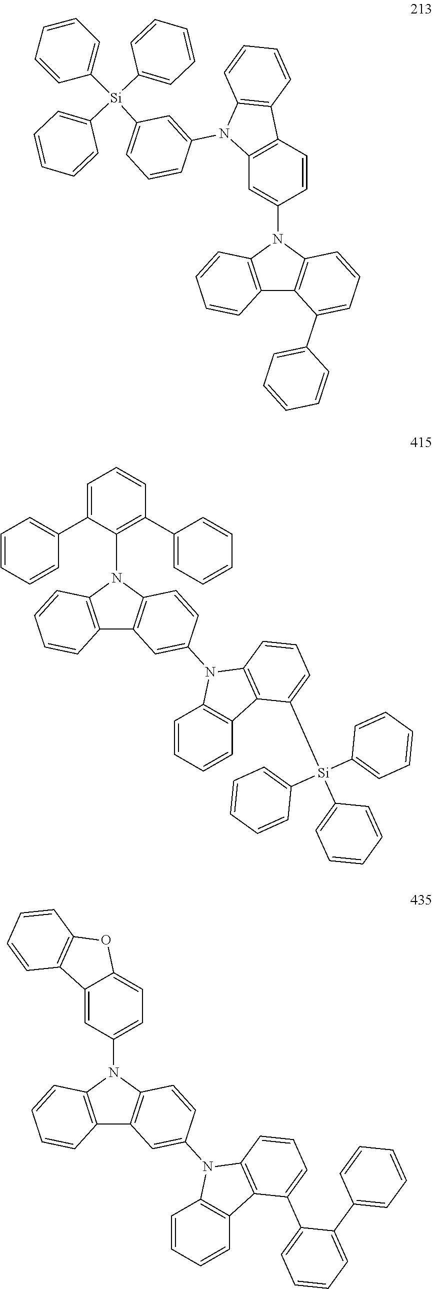 [SMILES: c1ccc(-c2cccc3c2c2ccccc2n3-c2ccc3c4ccccc4n(-c4cccc([Si](c5ccccc5)(c5ccccc5)c5ccccc5)c4)c3c2)cc1 ; c1ccc(-c2cccc(-c3ccccc3)c2-n2c3ccccc3c3cc(-n4c5ccccc5c5c([Si](c6ccccc6)(c6ccccc6)c6ccccc6)cccc54)ccc32)cc1 ; c1ccc(-c2ccccc2-c2cccc3c2c2ccccc2n3-c2ccc3c(c2)c2ccccc2n3-c2ccc3oc4ccccc4c3c2)cc1]

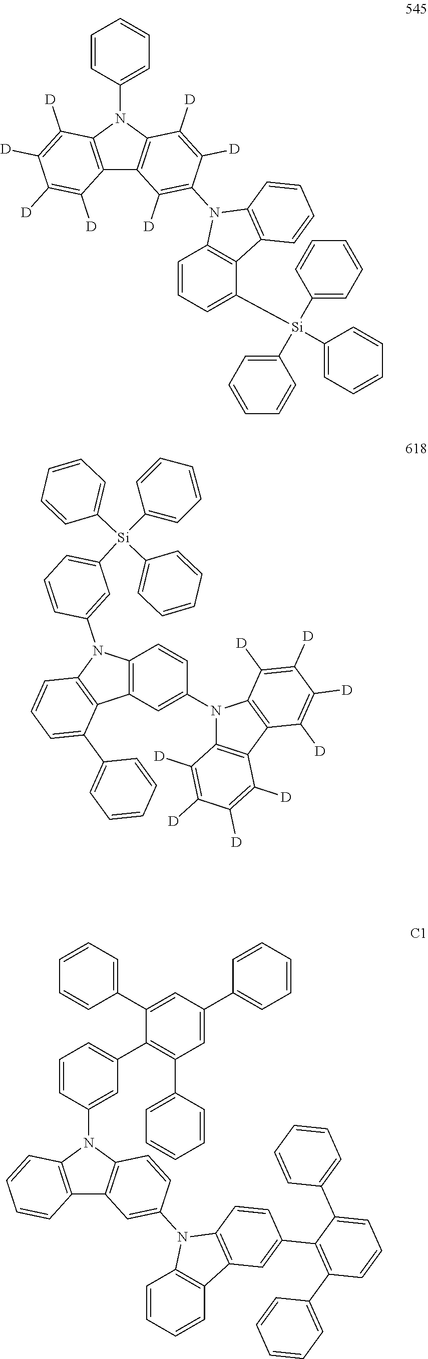 [SMILES: [2H]c1c([2H])c([2H])c2c(c1[2H])c1c([2H])c(-n3c4ccccc4c4c([Si](c5ccccc5)(c5ccccc5)c5ccccc5)cccc43)c([2H])c([2H])c1n2-c1ccccc1 ; [2H]c1c([2H])c([2H])c2c(c1[2H])c1c([2H])c([2H])c([2H])c([2H])c1n2-c1ccc2c(c1)c1c(-c3ccccc3)cccc1n2-c1cccc([Si](c2ccccc2)(c2ccccc2)c2ccccc2)c1 ; c1ccc(-c2cc(-c3ccccc3)c(-c3cccc(-n4c5ccccc5c5cc(-n6c7ccccc7c7cc(-c8c(-c9ccccc9)cccc8-c8ccccc8)ccc76)ccc54)c3)c(-c3ccccc3)c2)cc1]

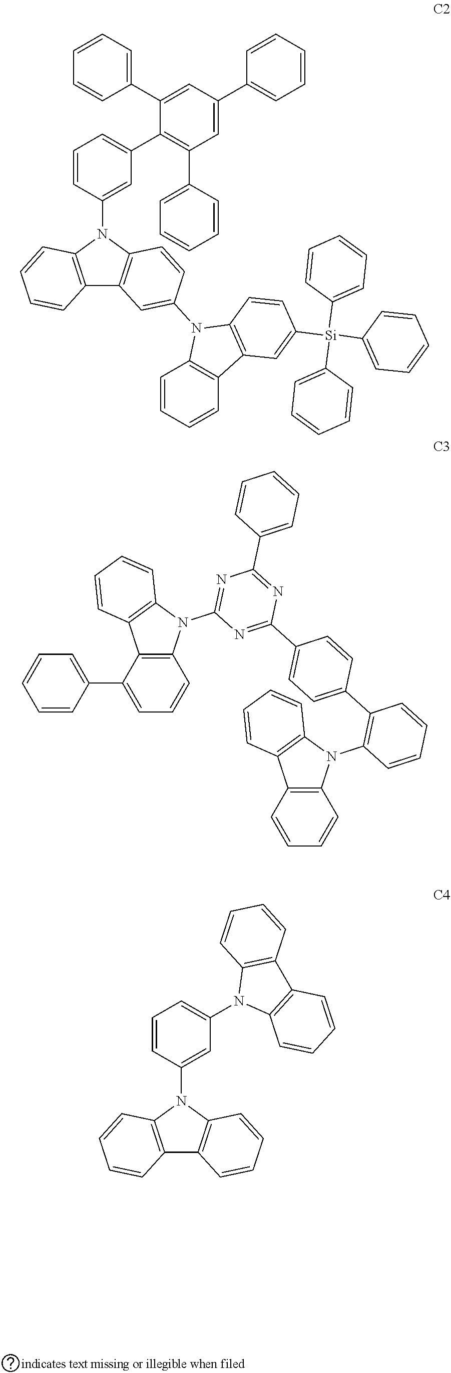 [SMILES: c1ccc(-c2cc(-c3ccccc3)c(-c3cccc(-n4c5ccccc5c5cc(-n6c7ccccc7c7cc([Si](c8ccccc8)(c8ccccc8)c8ccccc8)ccc76)ccc54)c3)c(-c3ccccc3)c2)cc1 ; c1ccc(-c2nc(-c3ccc(-c4ccccc4-n4c5ccccc5c5ccccc54)cc3)nc(-n3c4ccccc4c4c(-c5ccccc5)cccc43)n2)cc1 ; c1cc(-n2c3ccccc3c3ccccc32)cc(-n2c3ccccc3c3ccccc32)c1]

(Manufacture of Light Emitting Device)

For manufacturing the light emitting devices of the Examples and Comparative Examples, a glass substrate (product of Corning Inc.) on which an ITO electrode with 15 Ω/cm² (1200 Å) was formed as an anode, was cut into a size of about 50 mm×50 mm×0.7 mm, washed by ultrasonic waves utilizing isopropyl alcohol and distilled water for about 5 minutes each, and cleaned by irradiating ultraviolet rays for about 30 minutes and then, ozone. After that, the ITO glass substrate was installed in a vacuum deposition apparatus.

On the anode, a hole injection layer with a thickness of about 100 Å was formed by depositing HATCN, and on the hole injection layer, a first hole transport layer with a thickness of about 600 Å was formed by depositing H-1-1. On the first hole transport layer, a second hole transport layer with a thickness of about 50 Å was formed by depositing SiCzCz, to form a hole transport layer.

Then, the Example Compound according to one or more embodiments or Comparative Compound, ETH66 as a second compound, and AD-39 of a third compound were co-deposited in a weight ratio of about 60:27:13 to form an emission layer with a thickness of about 200 Å. On the emission layer, a first electron transport layer with a thickness of about 50 Å was formed by depositing ETH2. Then, on the first electron transport layer, ETH2 and LiQ were deposited in a weight ratio of about 1:1 to form a second electron transport layer with a thickness of about 300 Å, to form an electron transport layer. Then, on the electron transport layer, an electron injection layer with a thickness of about 15 Å was formed by depositing LiF. Then, on the electron injection layer, Al was deposited to form a cathode with a thickness of about 80 Å. All layers were formed by a vacuum deposition method.

The compounds utilized for the manufacture of the light emitting devices of the Examples and Comparative Examples are shown below. The materials below were utilized after purchasing commercial products and performing sublimation purification.

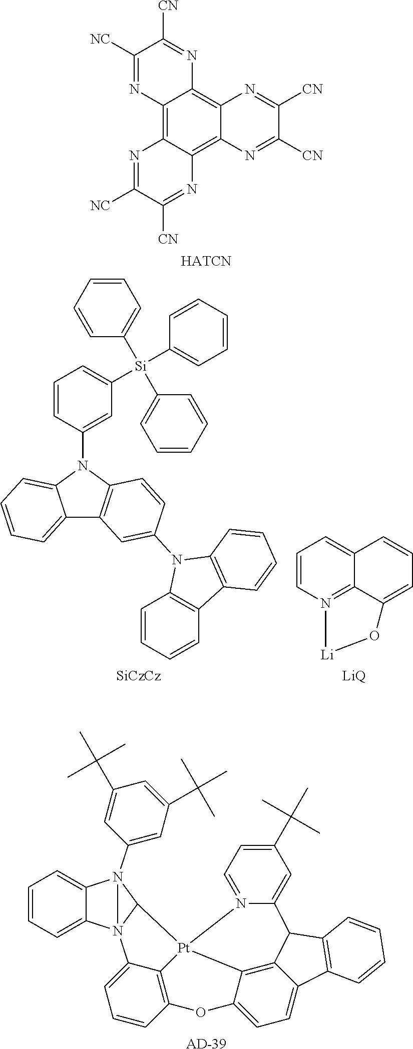

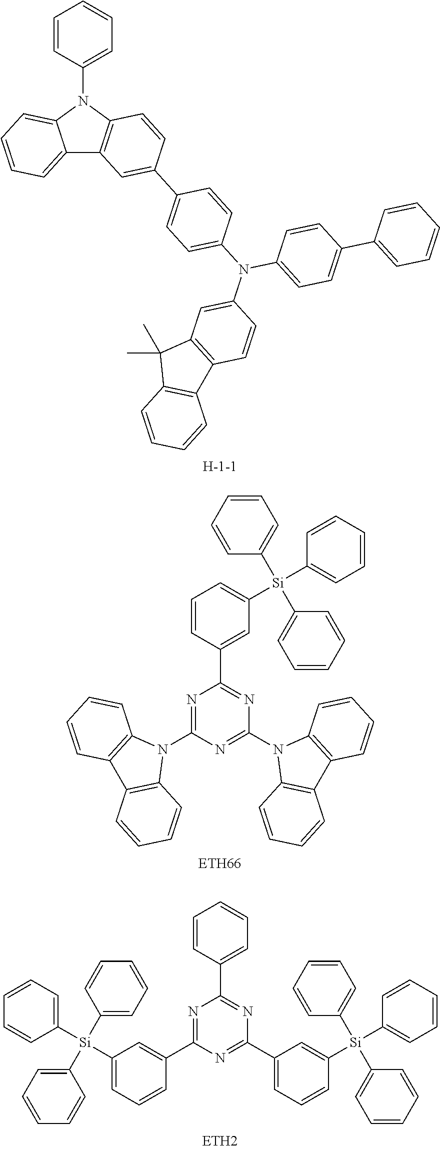 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1]

(Evaluation of Properties of Light Emitting Devices)

The device efficiency of the light emitting devices manufactured utilizing Example Compounds 1, 11, 182, 213, 415, 435, 545 and 618, and Comparative Compounds C1 to C4 were evaluated. In Table 2, the evaluation results for the light emitting devices of Examples 1 to 8, and Comparative Examples 1 to 4 are shown. In order to evaluate the properties of the light emitting devices manufactured in Examples 1 to 8 and Comparative Examples 1 to 4, a driving voltage (V) at a current density of about 10 mA/cm², and maximum quantum efficiency (%) were measured utilizing a source meter (Keithley Instrument, 2400 series) and an external quantum efficiency measurement apparatus (Hamamatsu Photonics, 09920-2-12). In the evaluation of the maximum quantum efficiency (%), luminance/current density were measured, and the maximum quantum efficiency (%) was converted supposing angular luminance distribution introducing Lambertian surface. The evaluation results of the properties of the light emitting devices are shown in Table 2.

TABLE 2

| | | Driving | Maximum | |
|---|---|---|---|---|
| | | voltage | quantum | Emission |
| | First compound | (V) | efficiency (%) | color |
| Example 1 | Compound 1 | 4.6 | 26.8 | Blue |
| Example 2 | Compound 11 | 4.5 | 25.5 | Blue |
| Example 3 | Compound 182 | 5.1 | 26.3 | Blue |
| Example 4 | Compound 213 | 4.9 | 25.2 | Blue |
| Example 5 | Compound 415 | 5.0 | 26.4 | Blue |
| Example 6 | Compound 435 | 4.8 | 25.9 | Blue |
| Example 7 | Compound 545 | 4.7 | 27.1 | Blue |
| Example 8 | Compound 618 | 4.6 | 26.8 | Blue |
| Comparative | Comparative | 5.4 | 23.9 | Blue |
| Example 1 | Compound C1 | | | |
| Comparative | Comparative | 5.5 | 24.1 | Blue |
| Example 2 | Compound C2 | | | |
| Comparative | Comparative | 5.6 | 22.7 | Blue |
| Example 3 | Compound C3 | | | |
| Comparative | Comparative | 5.3 | 23.4 | Blue |
| Example 4 | Compound C4 | | | |

Referring to the results of Table 2, it could be confirmed that the Examples of the light emitting devices utilizing the nitrogen-containing compounds of embodiments of the present disclosure as light-emitting materials, showed lower driving voltages and improved color purity and efficiency, when compared to the Comparative Examples. The Example Compounds include a first carbazole group and a second carbazole group connected with the first carbazole group, and a first substituent including a phenyl moiety or a triphenylsilane moiety is bonded to the carbon of position 4 of the first and/or second carbazole group. Accordingly, the light emitting devices of embodiments including the Example Compounds may show reduced interaction between the Example Compound and a dopant, an increased triplet energy difference, improved color purity and life characteristics and improved emission efficiency. The light emitting device of one or more embodiments includes the Example Compounds as the light-emitting dopants of an emitting device (e.g., a thermally activated delayed fluorescence (TADF) emitting device), and may achieve high device efficiency, particularly in a blue wavelength region.

Referring to Comparative Example 1 and Comparative Example 2, in Comparative Compound C1, a first substituent including a phenyl moiety is bonded to the carbon of position 3 of a carbazole group, and in Comparative Compound C2, a first substituent including a triphenylsilane moiety is bonded to the carbon of position 3 of a carbazole group, and driving voltages for Comparative Example 1 and Comparative Example 2 were high and color purity was degraded when compared to the Examples. When the first substituent is substituted at the carbon of position 4 of a carbazole group as in the nitrogen-containing compound of one or more embodiments of the present disclosure, high emission efficiency and long lifetime may be achieved.

Referring to Comparative Example 3, in Comparative Compound 3, a first substituent is bonded to the carbon of position 4 of a carbazole group, but a linker such as triazine is included between a first carbazole group and a second carbazole group, and a driving voltage was high and color purity and efficiency were degraded when compared to the Examples. When the second carbazole group is connected with the benzene moiety of the first carbazole group as in the nitrogen-containing compound of one or more embodiments of the present disclosure, high emission efficiency and long lifetime may be achieved.

Referring to Comparative Example 4, in Comparative Compound C4, a first substituent is not bonded to a carbazole group, and a driving voltage was high and color purity and efficiency were degraded when compared to the Examples. When the first substituent is connected with the carbazole group as in the nitrogen-containing compound of one or more embodiments of the present disclosure, high emission efficiency and long lifetime may be achieved.

The light emitting device of one or more embodiments may show improved device properties of high efficiency and long lifetime.

The nitrogen-containing compound of one or more embodiments is included in an emission layer of a light emitting device and may contribute to the increase of the efficiency and lifetime of the light emitting device.

Although the embodiments of the present disclosure have been described, it is understood that the present disclosure should not be limited to these embodiments, but one or more suitable changes and modifications can be made by one ordinary skilled in the art within the spirit and scope of the present disclosure as hereinafter claimed by the following claims and their equivalents.

What is claimed is:

1. A light emitting device, comprising:
a first electrode;
a second electrode opposite to the first electrode; and
an emission layer between the first electrode and the second electrode,
wherein the emission layer comprises a first compound represented by Formula 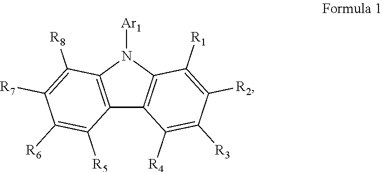
wherein in Formula 1,
Ar₁ is a substituted or unsubstituted aryl group of 6 to 30 ring-forming carbon atoms, or a substituted or unsubstituted heteroaryl group of 2 to 30 ring-forming carbon atoms,
R₁ to R₈ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
any one among R₅ to R₈ is a substituent represented by Formula 2: 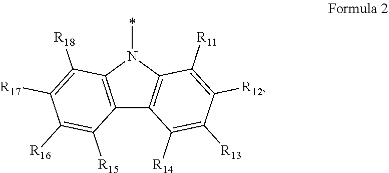
wherein in Formula 2,
R₁₁ to R₁₈ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
wherein in Formula 1 and Formula 2, at least one of R₄ or R₁₄ is a substituted or unsubstituted phenyl group or a substituent represented by Formula 3: 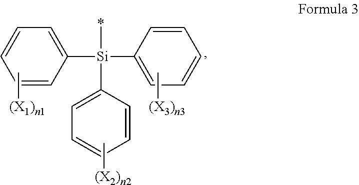
wherein in Formula 3,
X₁ to X₃ are each independently a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and
n1 to n3 are each independently an integer of 0 to 5.

2. The light emitting device of claim 1, wherein the first compound represented by Formula 1 is represented by Formula 1-1: 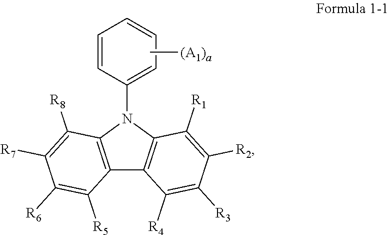
and
wherein in Formula 1-1,
A₁ is a hydrogen atom, a deuterium atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl, or a substituted or unsubstituted biphenyl group, and/or combined with an adjacent group to form a ring,
“a” is an integer of 0 to 5, and
R₁ to R₈ are as defined in Formula 1.

3. The light emitting device of claim 2, wherein, in Formula 1-1, A₁ is a substituent represented by any one of Formula 4-1 to Formula 4-11: 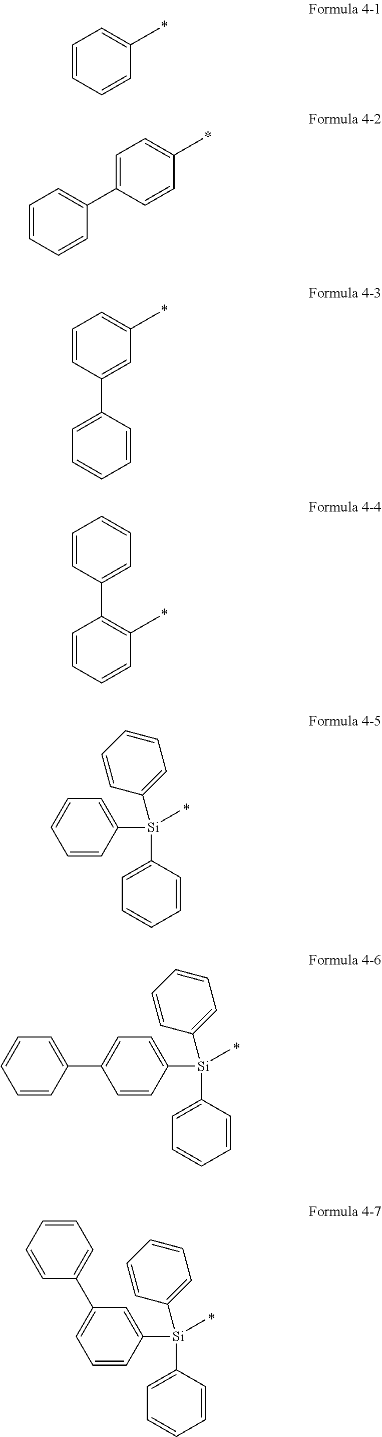 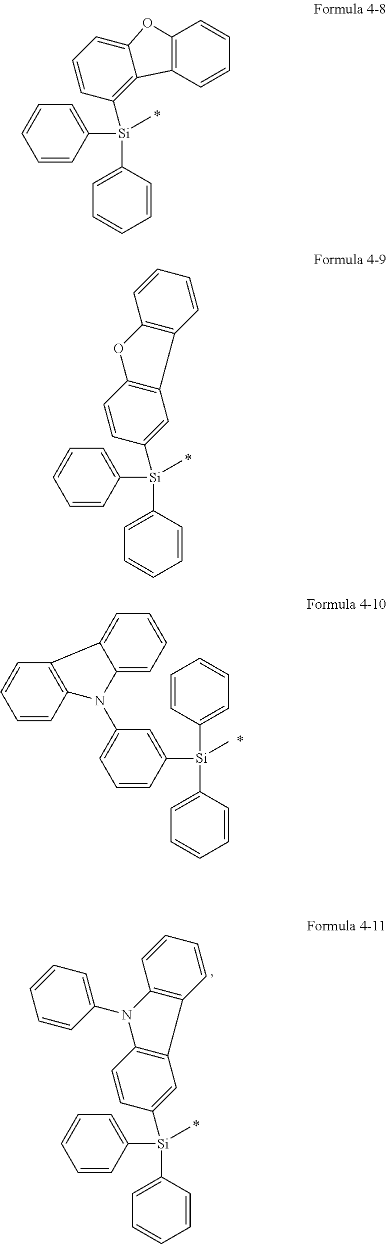
and
wherein in Formula 4-1 to Formula 4-11,
“ [symbol] ” is a position connected with Formula 1-1.

4. The light emitting device of claim 1, wherein the first compound represented by Formula 1 is represented by Formula 1-2: 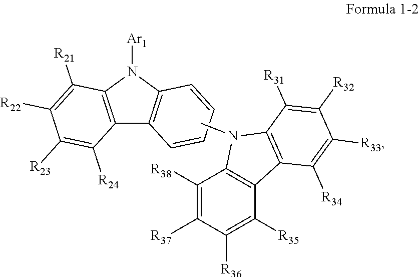
and
wherein in Formula 1-2,
R₂₁ to R₃₈ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
at least one of R₂₄ or R₃4 is a substituted or unsubstituted phenyl group, or a substituent represented by Formula 3, and
Ar₁ is as defined in Formula 1.

5. The light emitting device of claim 1, wherein the substituent represented by Formula 3 is represented by Formula 3-1: 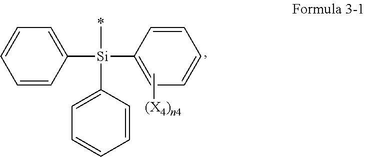
and
wherein in Formula 3-1,
X₄ is a hydrogen atom, a deuterium atom, a cyano group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl group, or a substituted or unsubstituted heteroaryl group of 2 to 10 ring-forming carbon atoms, and/or combined with an adjacent group to form a ring, and
n4 is an integer of 0 to 5.

6. The light emitting device of claim 1, wherein the first compound represented by Formula 1 is represented by Formula 1-3 or Formula 1-4: 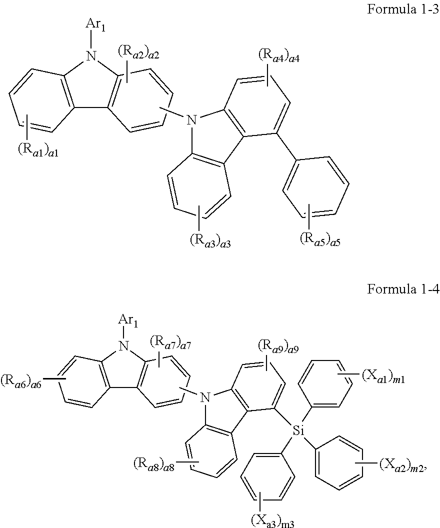
and
wherein in Formula 1-3 and Formula 1-4,
Rₐ₁ to Rₐ₉ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Xₐ₁ to Xₐ₃ are each independently a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
a1, a3, a6 and a8 are each independently an integer of 0 to 4,
a2, a4, a7 and a9 are each independently an integer of 0 to 3,
a5 and m1 to m3 are each independently an integer of 0 to 5, and
Ar₁ is as defined in Formula 1.

7. The light emitting device of claim 6, wherein Rₐ₅ and Xₐ₁ to Xₐ₃ are each independently a hydrogen atom, a deuterium atom, or a substituted or unsubstituted phenyl group.

8. The light emitting device of claim 1, wherein the first compound represented by Formula 1 is represented by Formula 1-5 or Formula 1-6: 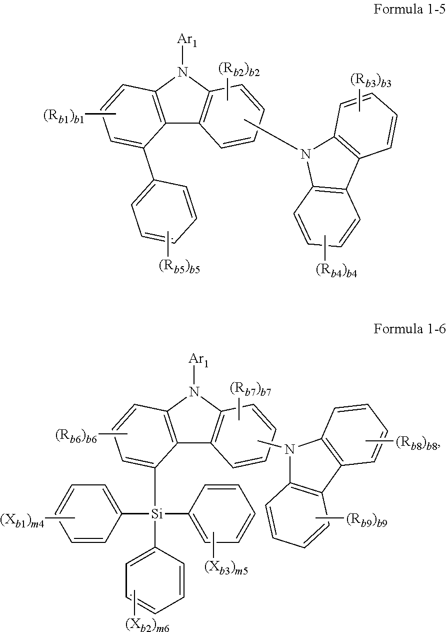
and
wherein in Formula 1-5 and Formula 1-6,
R_{b1} to R_{b9} are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
X_{b1} to X_{b3} are each independently a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
b1, b2, b6 and b7 are each independently an integer of 0 to 3,
b3, b4, b8 and b9 are each independently an integer of 0 to 4,
b5 and m4 to m6 are each independently an integer of 0 to 5, and
Ar₁ is as defined in Formula 1.

9. The light emitting device of claim 8, wherein R_{b3}, R_{b4}, R_{b8} and R_{b9} are each independently a hydrogen atom, a deuterium atom, a substituted or unsubstituted phenyl group, or a substituted or unsubstituted silyl group.

10. The light emitting device of claim 1, wherein the first compound represented by Formula 1 comprises at least one among compounds represented in Compound Group 1:

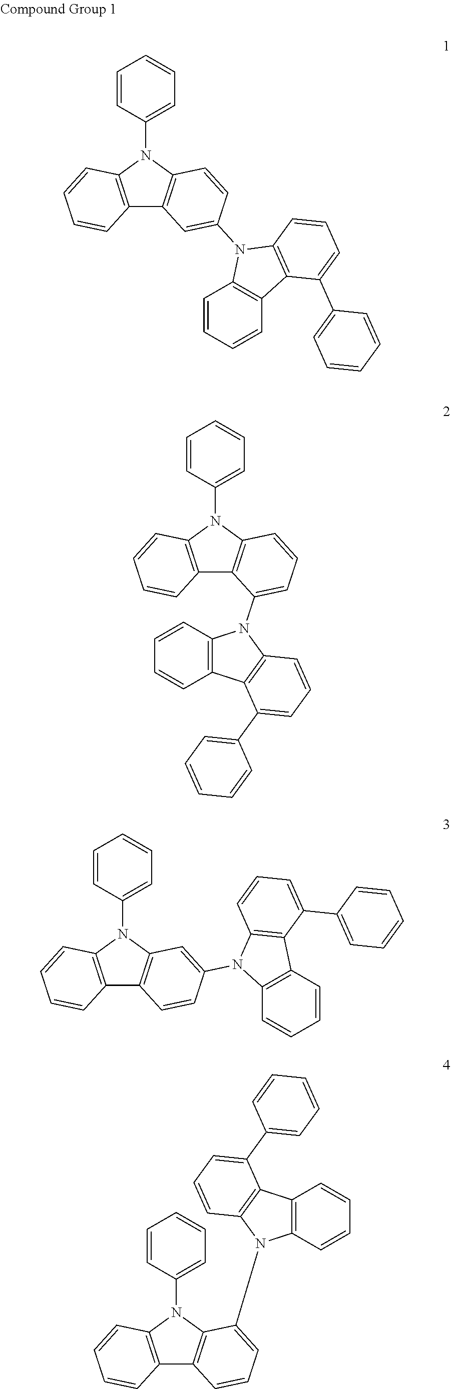

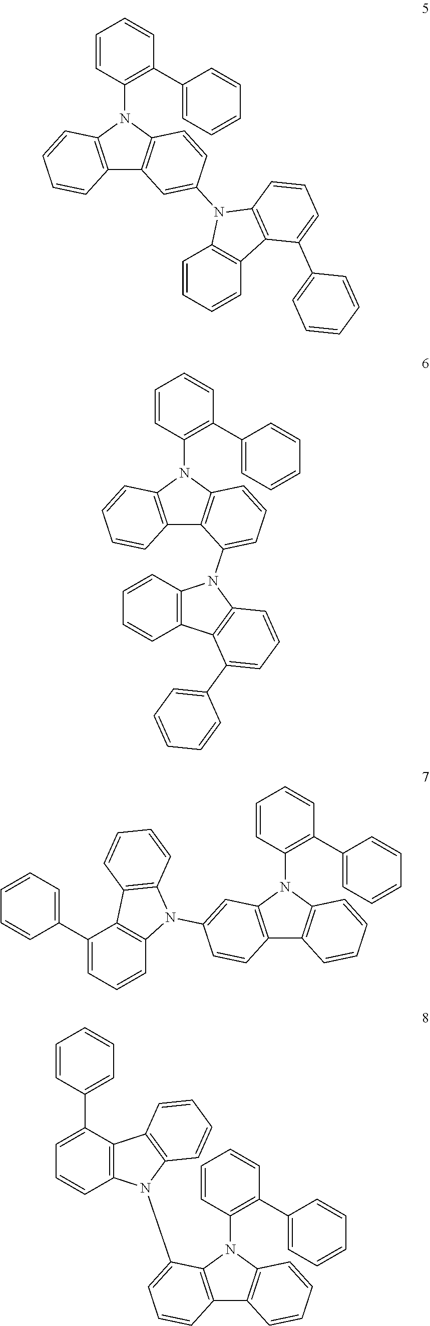 [SMILES: c1ccc(-c2ccccc2-n2c3ccccc3c3cc(-n4c5ccccc5c5c(-c6ccccc6)cccc54)ccc32)cc1 ; c1ccc(-c2ccccc2-n2c3ccccc3c3c(-n4c5ccccc5c5c(-c6ccccc6)cccc54)cccc32)cc1 ; c1ccc(-c2ccccc2-n2c3ccccc3c3ccc(-n4c5ccccc5c5c(-c6ccccc6)cccc54)cc32)cc1 ; c1ccc(-c2ccccc2-n2c3ccccc3c3cccc(-n4c5ccccc5c5c(-c6ccccc6)cccc54)c32)cc1]

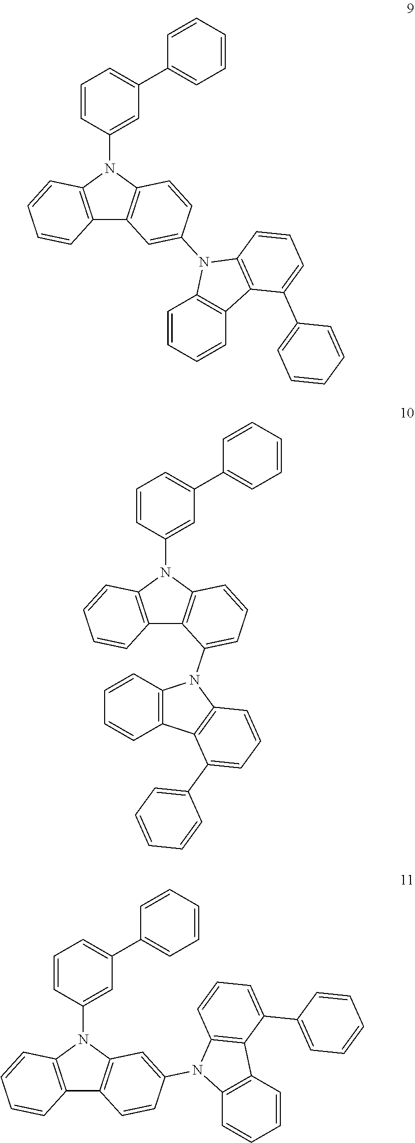

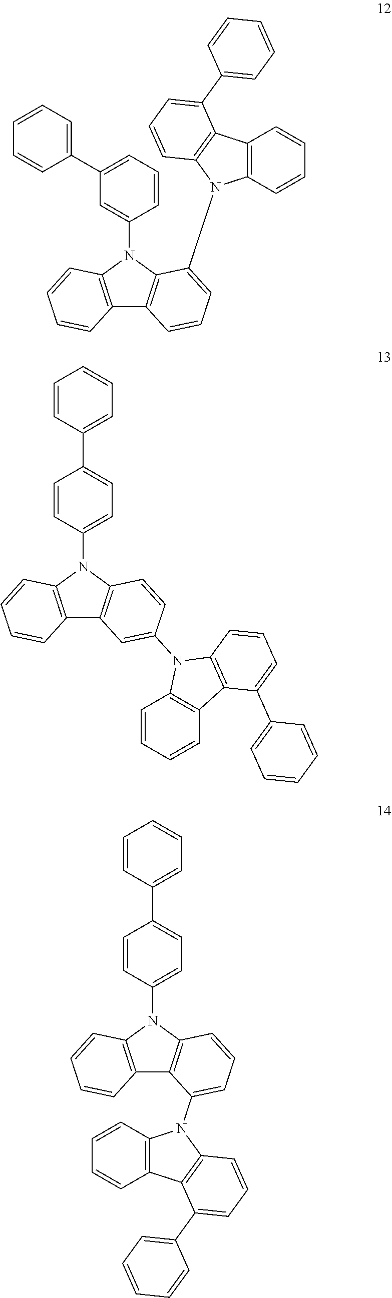

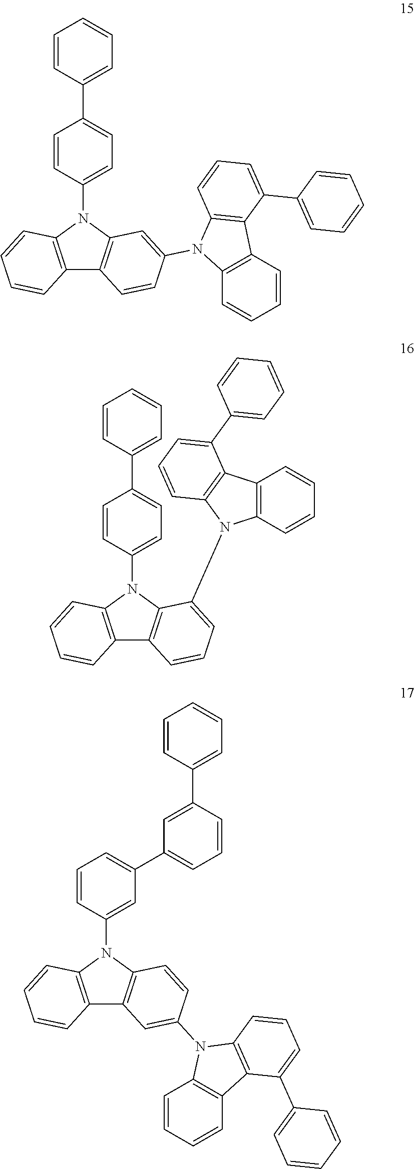

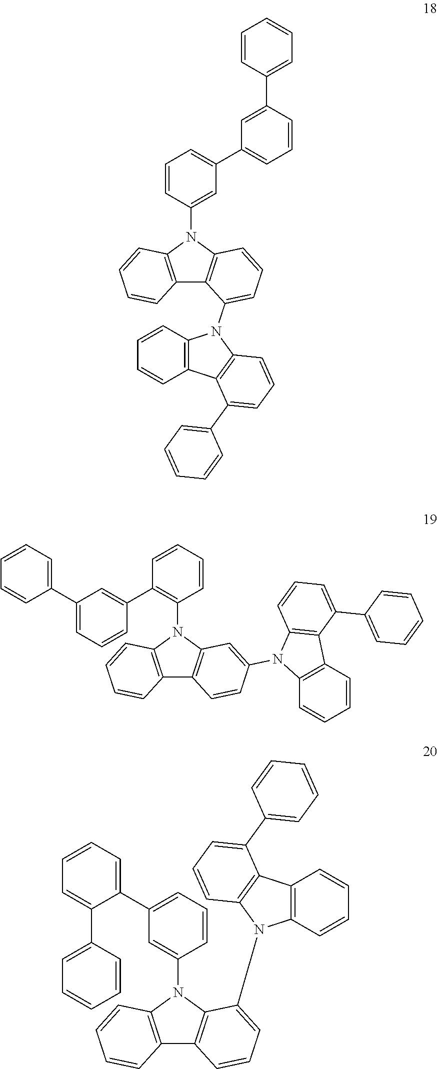 [SMILES: c1ccc(-c2cccc(-c3cccc(-n4c5ccccc5c5c(-n6c7ccccc7c7c(-c8ccccc8)cccc76)cccc54)c3)c2)cc1 ; c1ccc(-c2cccc(-c3ccccc3-n3c4ccccc4c4ccc(-n5c6ccccc6c6c(-c7ccccc7)cccc65)cc43)c2)cc1 ; c1ccc(-c2ccccc2-c2cccc(-n3c4ccccc4c4cccc(-n5c6ccccc6c6c(-c7ccccc7)cccc65)c43)c2)cc1]

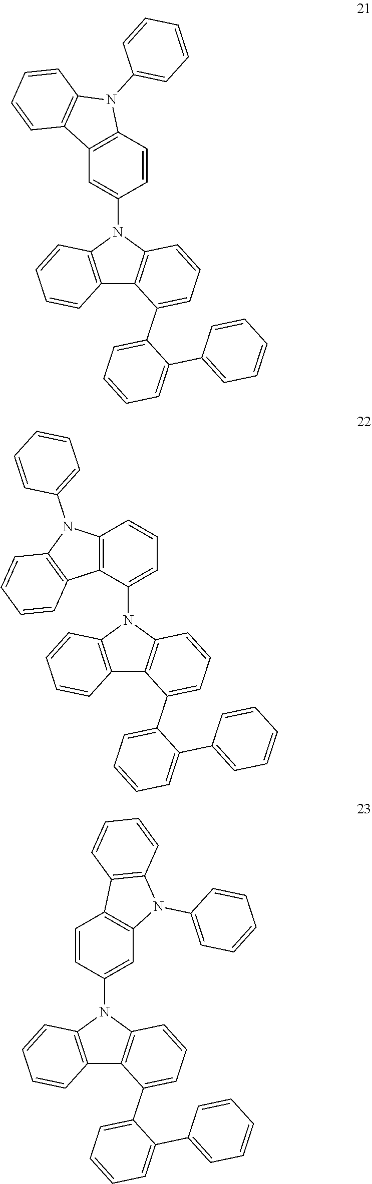

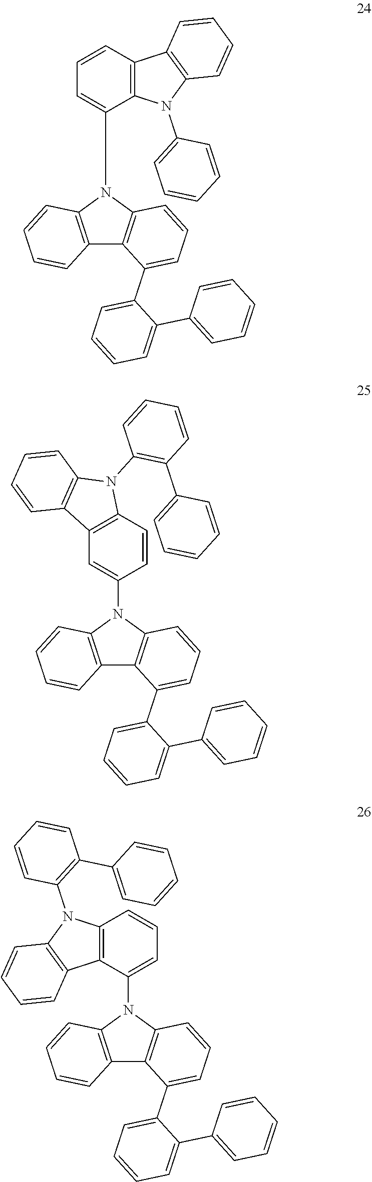

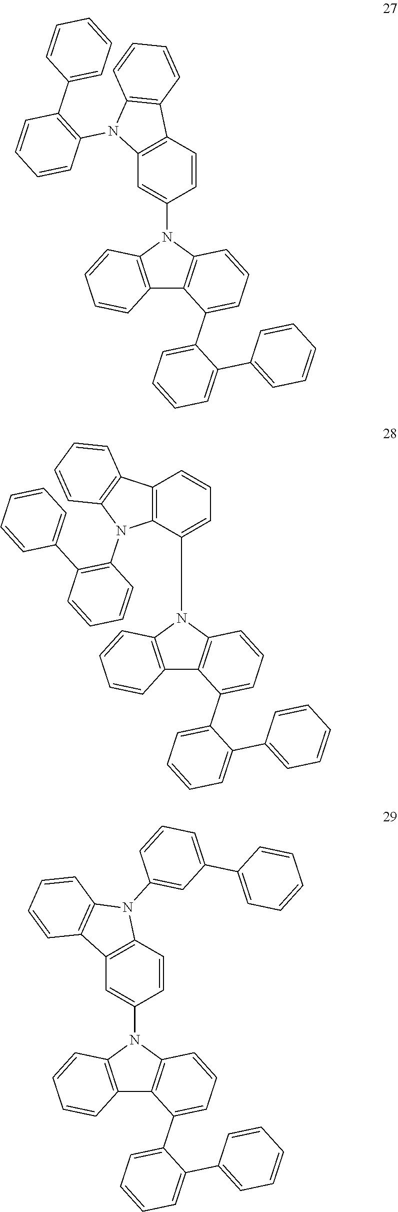

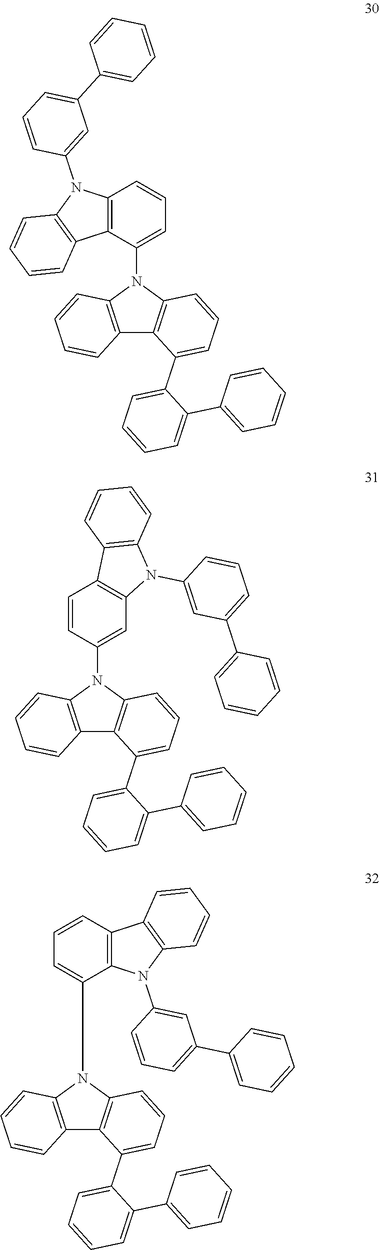

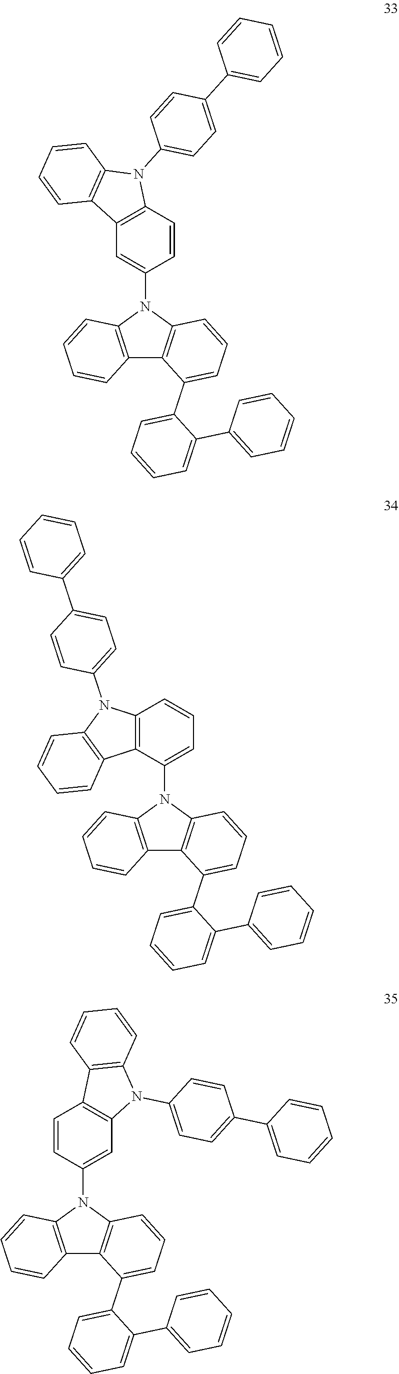 [SMILES: c1ccc(-c2ccc(-n3c4ccccc4c4cc(-n5c6ccccc6c6c(-c7ccccc7-c7ccccc7)cccc65)ccc43)cc2)cc1 ; c1ccc(-c2ccc(-n3c4ccccc4c4c(-n5c6ccccc6c6c(-c7ccccc7-c7ccccc7)cccc65)cccc43)cc2)cc1 ; c1ccc(-c2ccc(-n3c4ccccc4c4ccc(-n5c6ccccc6c6c(-c7ccccc7-c7ccccc7)cccc65)cc43)cc2)cc1]

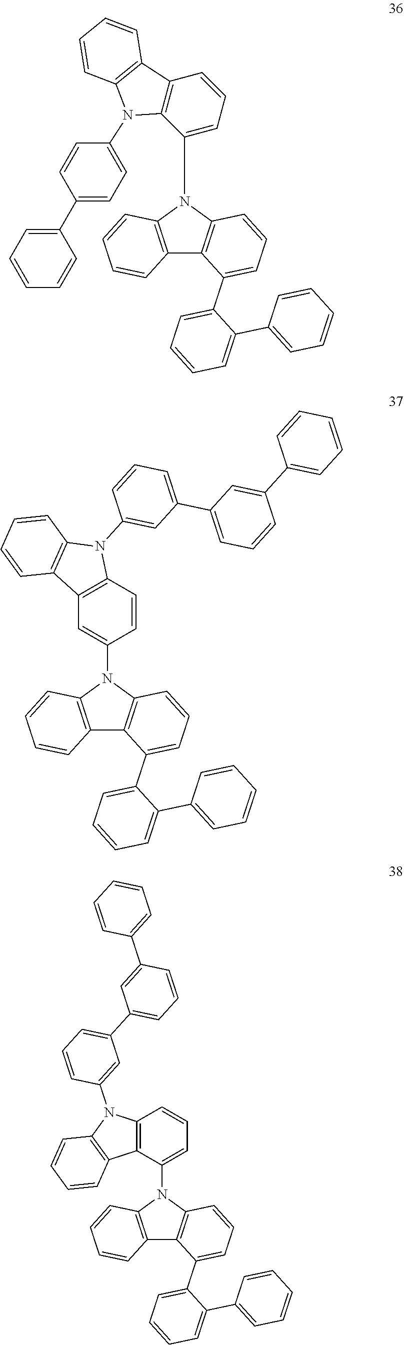

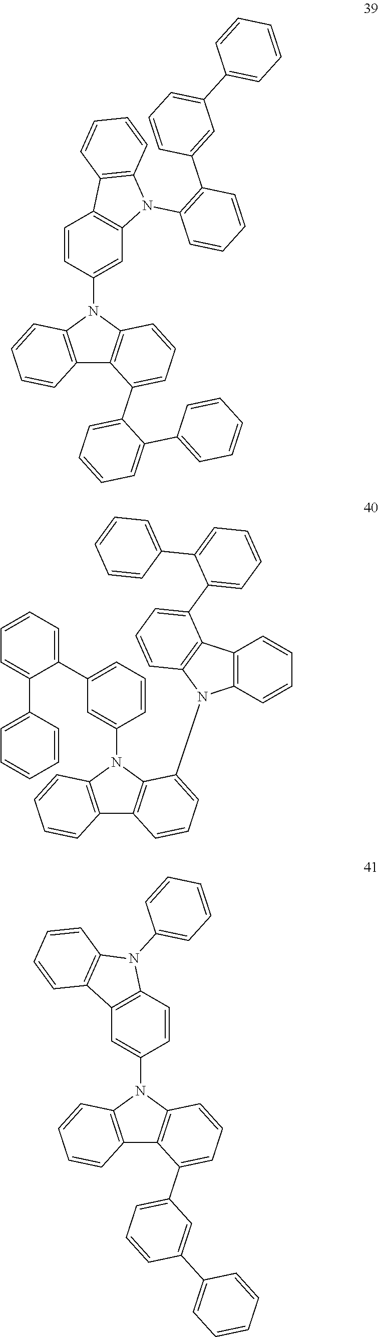

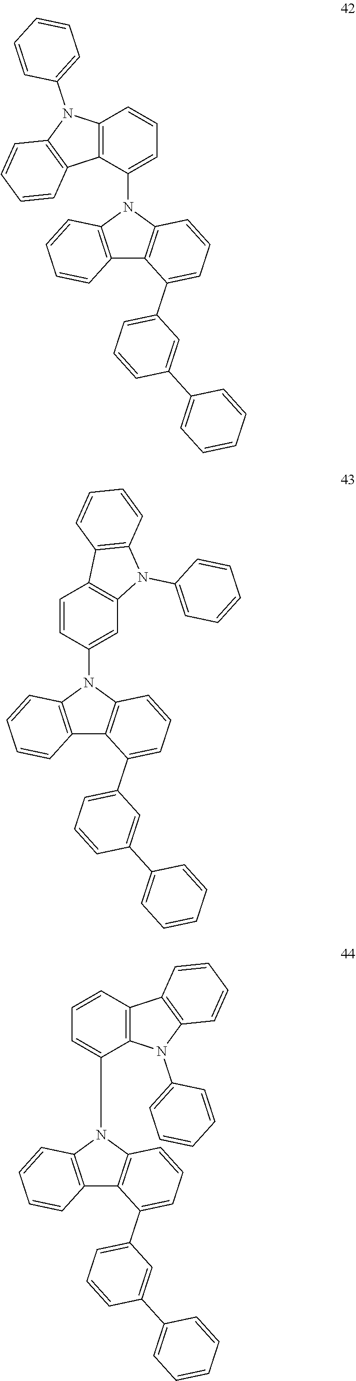

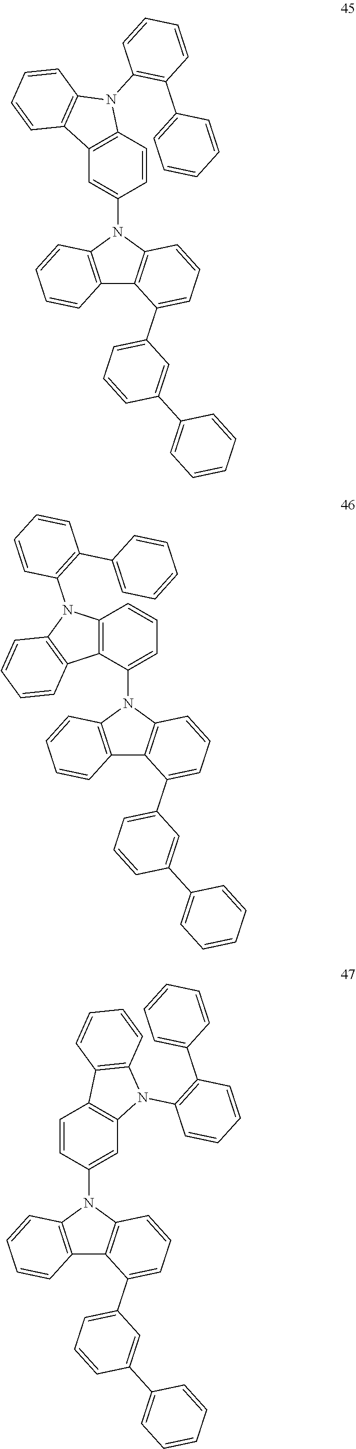

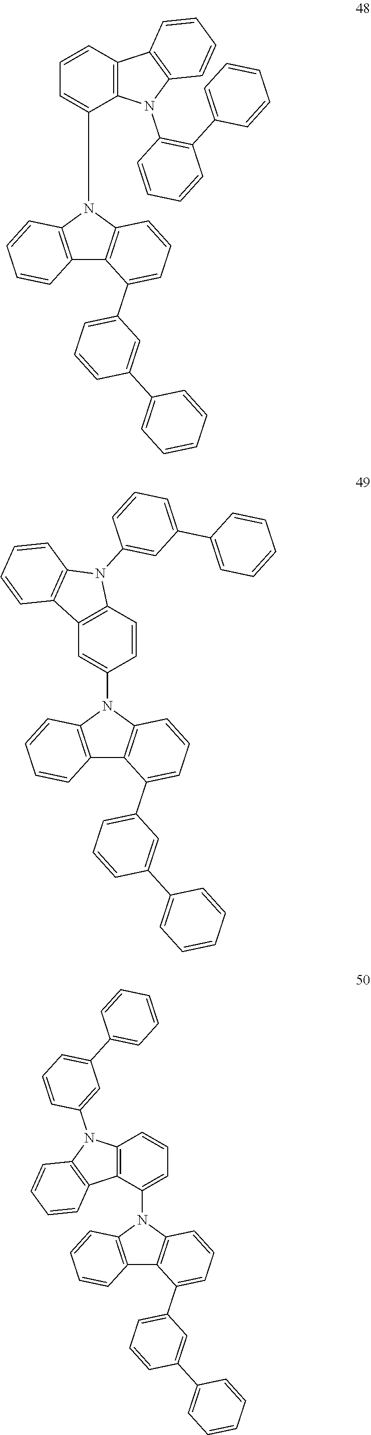

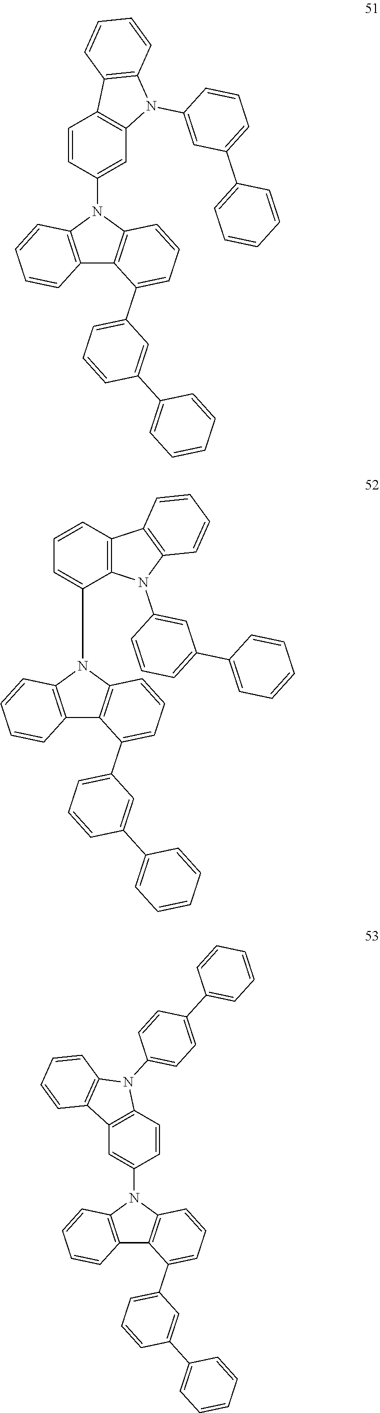

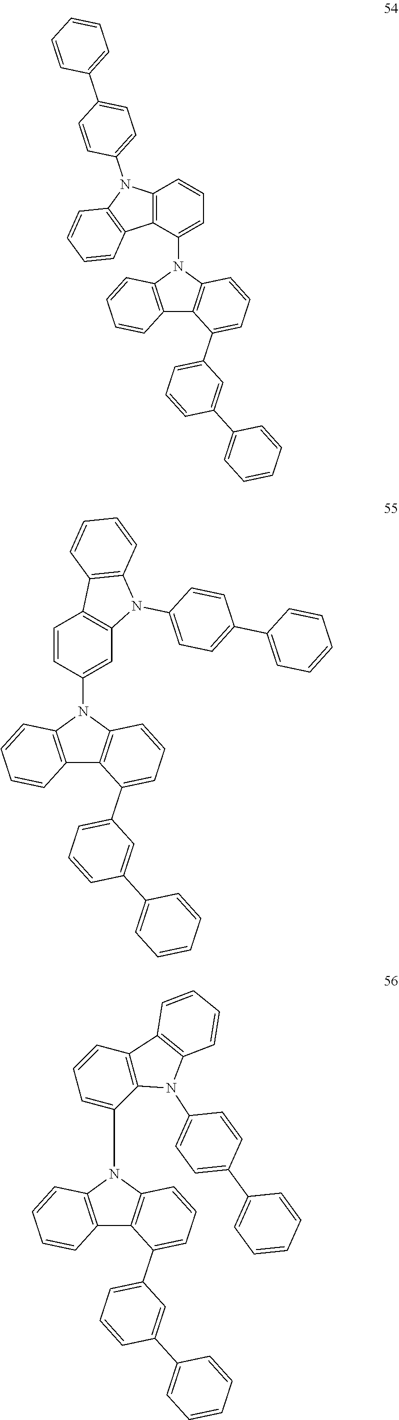

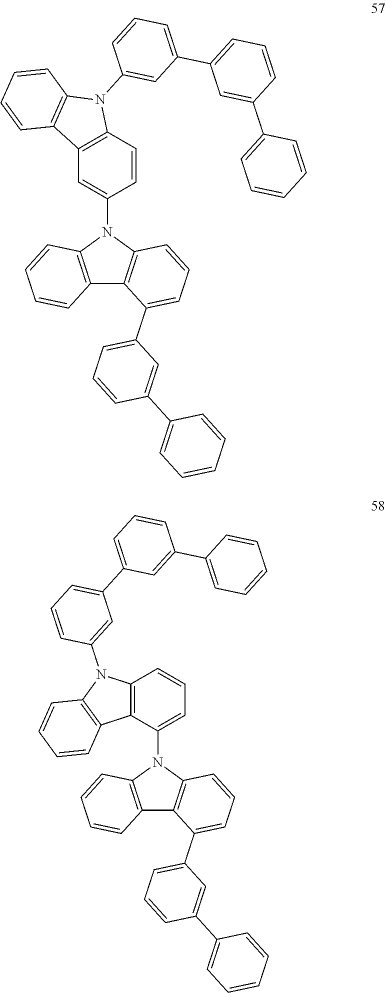 [SMILES: c1ccc(-c2cccc(-c3cccc(-n4c5ccccc5c5cc(-n6c7ccccc7c7c(-c8cccc(-c9ccccc9)c8)cccc76)ccc54)c3)c2)cc1 ; c1ccc(-c2cccc(-c3cccc(-n4c5ccccc5c5c(-n6c7ccccc7c7c(-c8cccc(-c9ccccc9)c8)cccc76)cccc54)c3)c2)cc1]

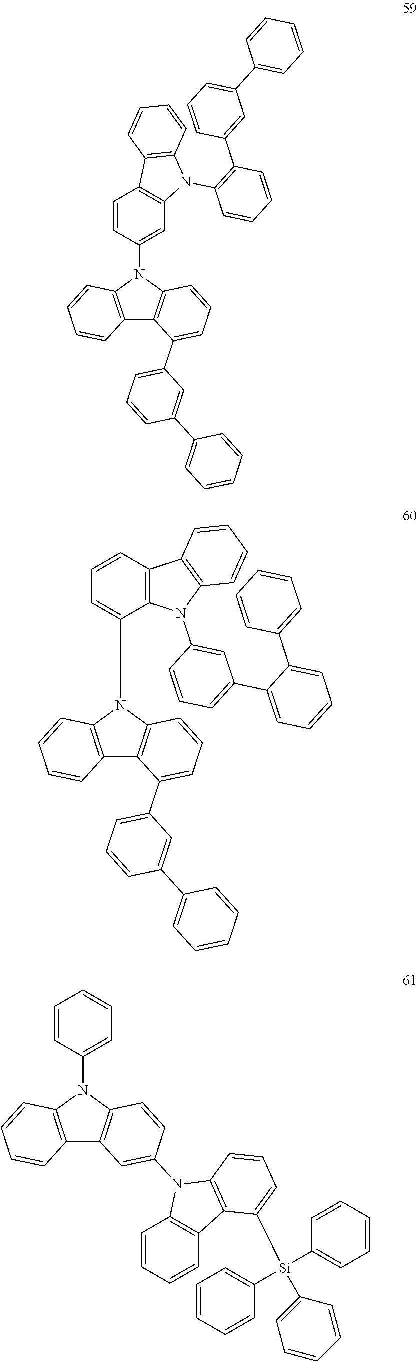

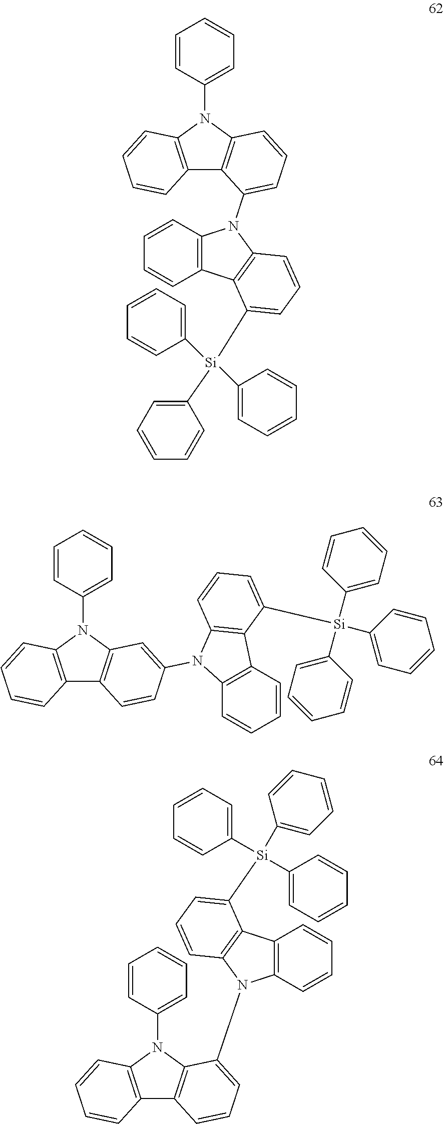 [SMILES: c1ccc(-n2c3ccccc3c3c(-n4c5ccccc5c5c([Si](c6ccccc6)(c6ccccc6)c6ccccc6)cccc54)cccc32)cc1 ; c1ccc(-n2c3ccccc3c3ccc(-n4c5ccccc5c5c([Si](c6ccccc6)(c6ccccc6)c6ccccc6)cccc54)cc32)cc1 ; c1ccc(-n2c3ccccc3c3cccc(-n4c5ccccc5c5c([Si](c6ccccc6)(c6ccccc6)c6ccccc6)cccc54)c32)cc1]

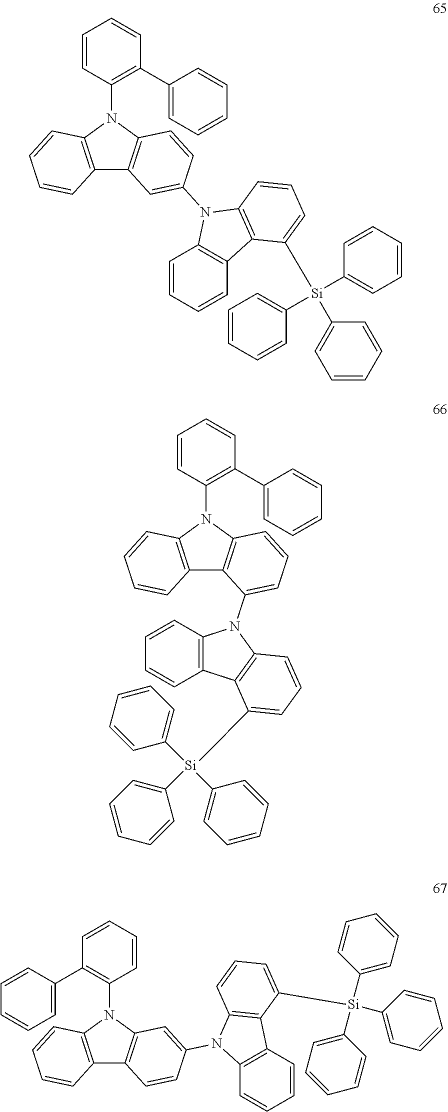 [SMILES: c1ccc(-c2ccccc2-n2c3ccccc3c3cc(-n4c5ccccc5c5c([Si](c6ccccc6)(c6ccccc6)c6ccccc6)cccc54)ccc32)cc1 ; c1ccc(-c2ccccc2-n2c3ccccc3c3c(-n4c5ccccc5c5c([Si](c6ccccc6)(c6ccccc6)c6ccccc6)cccc54)cccc32)cc1 ; c1ccc(-c2ccccc2-n2c3ccccc3c3ccc(-n4c5ccccc5c5c([Si](c6ccccc6)(c6ccccc6)c6ccccc6)cccc54)cc32)cc1]

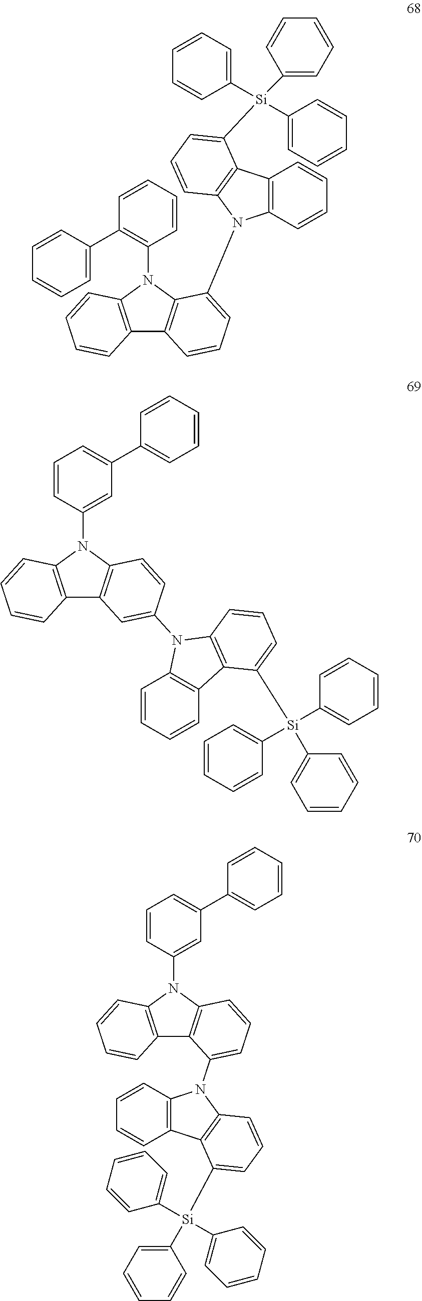

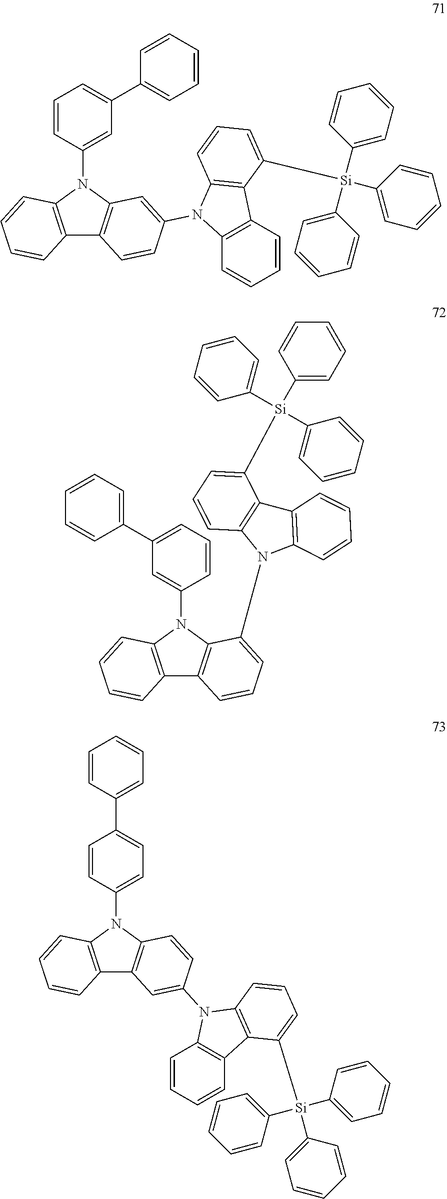

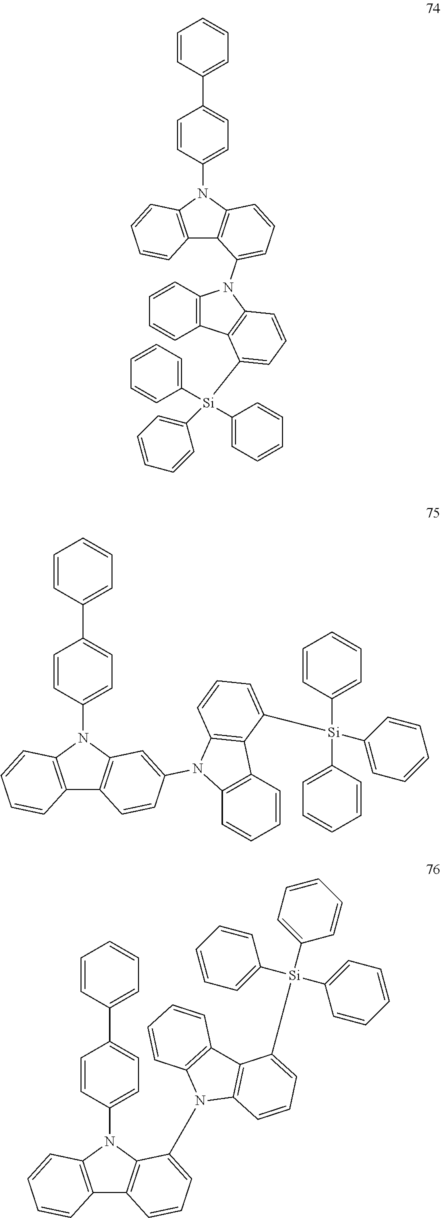 [SMILES: c1ccc(-c2ccc(-n3c4ccccc4c4c(-n5c6ccccc6c6c([Si](c7ccccc7)(c7ccccc7)c7ccccc7)cccc65)cccc43)cc2)cc1 ; c1ccc(-c2ccc(-n3c4ccccc4c4ccc(-n5c6ccccc6c6c([Si](c7ccccc7)(c7ccccc7)c7ccccc7)cccc65)cc43)cc2)cc1 ; c1ccc(-c2ccc(-n3c4ccccc4c4cccc(-n5c6ccccc6c6c([Si](c7ccccc7)(c7ccccc7)c7ccccc7)cccc65)c43)cc2)cc1]

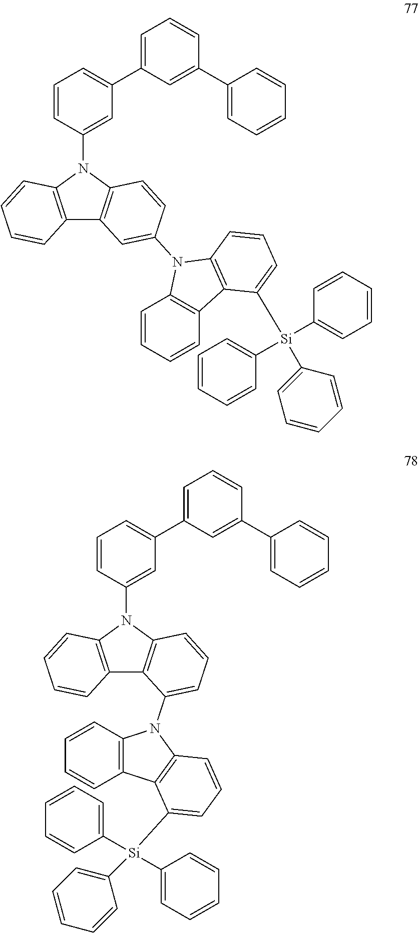 [SMILES: c1ccc(-c2cccc(-c3cccc(-n4c5ccccc5c5cc(-n6c7ccccc7c7c([Si](c8ccccc8)(c8ccccc8)c8ccccc8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8ccccc8)cccc76)cccc54)c3)c2)cc1]

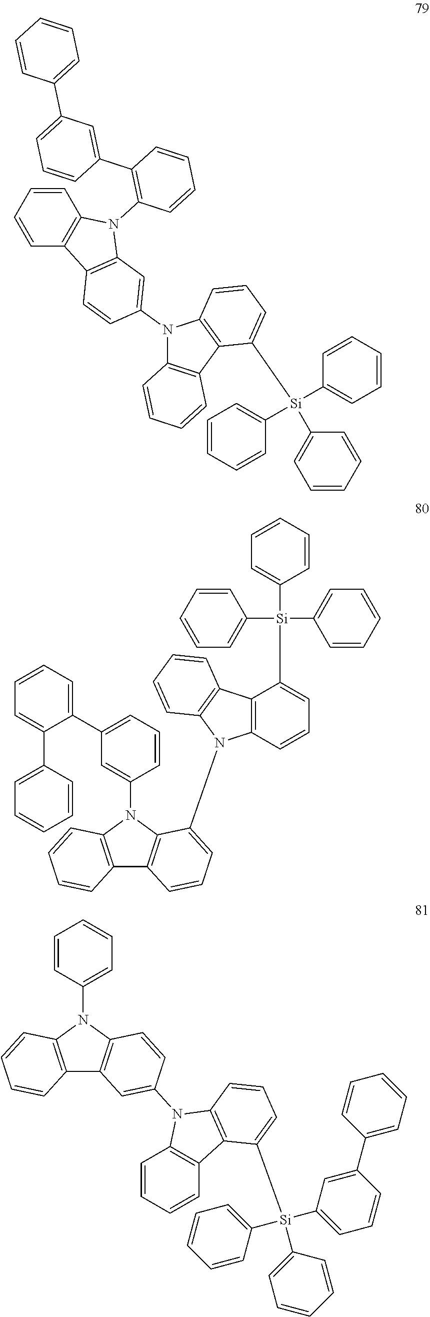

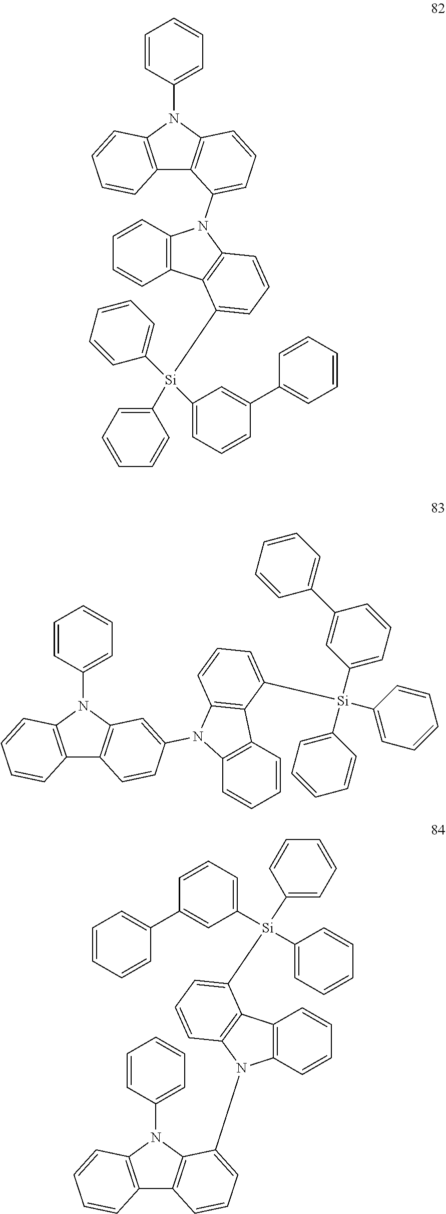

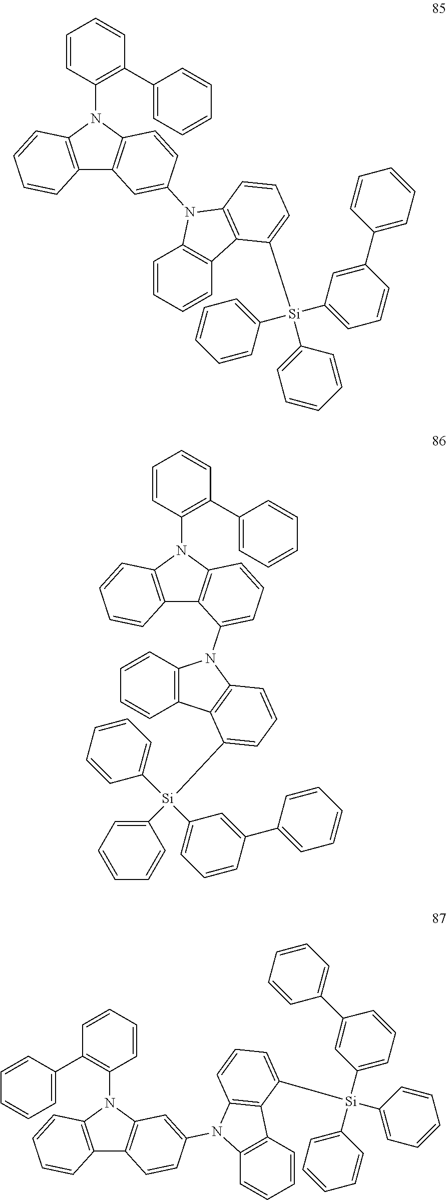 [SMILES: c1ccc(-c2cccc([Si](c3ccccc3)(c3ccccc3)c3cccc4c3c3ccccc3n4-c3ccc4c(c3)c3ccccc3n4-c3ccccc3-c3ccccc3)c2)cc1 ; c1ccc(-c2cccc([Si](c3ccccc3)(c3ccccc3)c3cccc4c3c3ccccc3n4-c3cccc4c3c3ccccc3n4-c3ccccc3-c3ccccc3)c2)cc1 ; c1ccc(-c2cccc([Si](c3ccccc3)(c3ccccc3)c3cccc4c3c3ccccc3n4-c3ccc4c5ccccc5n(-c5ccccc5-c5ccccc5)c4c3)c2)cc1]

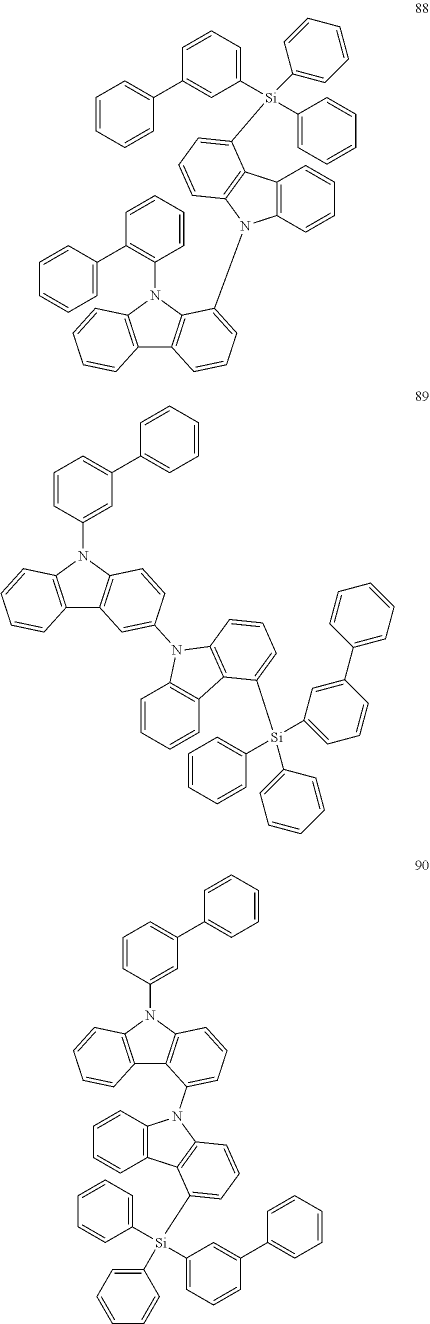

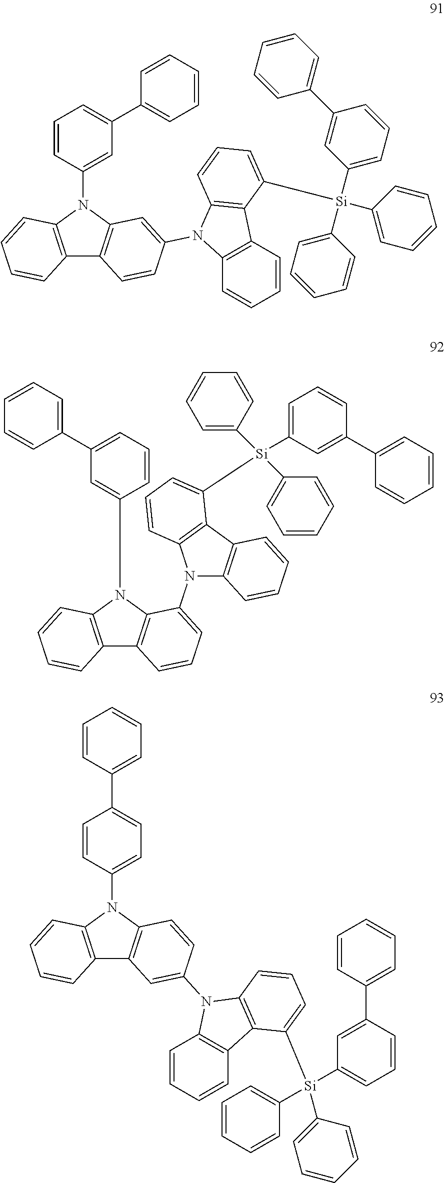 [SMILES: c1ccc(-c2cccc(-n3c4ccccc4c4ccc(-n5c6ccccc6c6c([Si](c7ccccc7)(c7ccccc7)c7cccc(-c8ccccc8)c7)cccc65)cc43)c2)cc1 ; c1ccc(-c2cccc(-n3c4ccccc4c4cccc(-n5c6ccccc6c6c([Si](c7ccccc7)(c7ccccc7)c7cccc(-c8ccccc8)c7)cccc65)c43)c2)cc1 ; c1ccc(-c2ccc(-n3c4ccccc4c4cc(-n5c6ccccc6c6c([Si](c7ccccc7)(c7ccccc7)c7cccc(-c8ccccc8)c7)cccc65)ccc43)cc2)cc1]

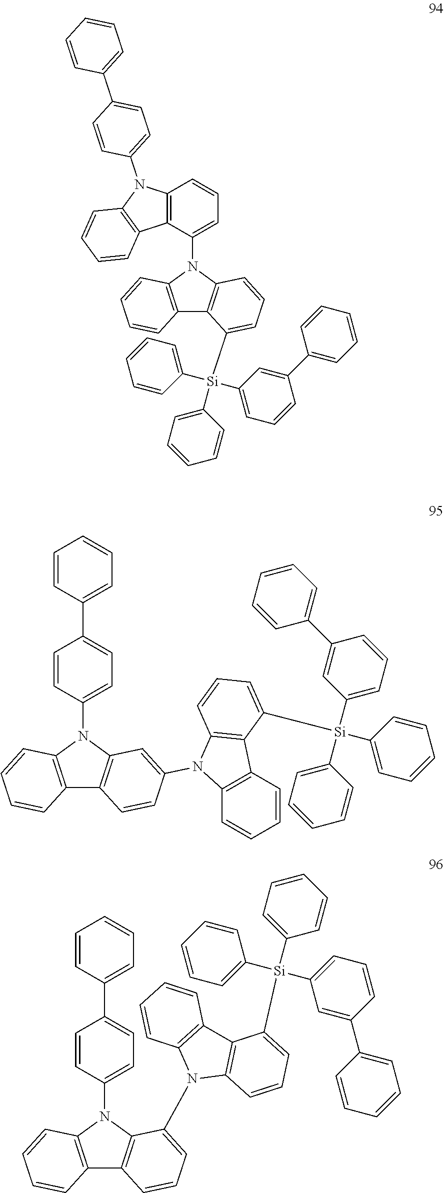

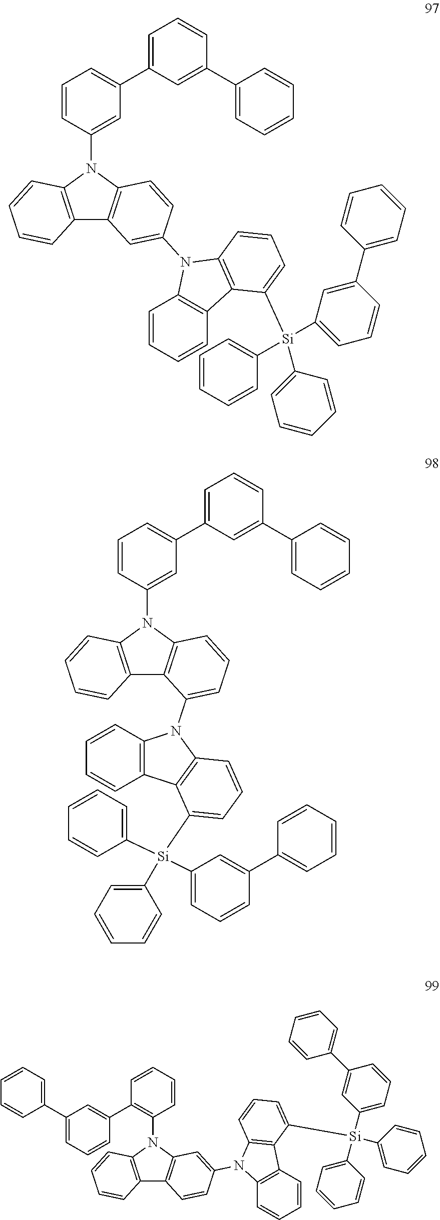 [SMILES: c1ccc(-c2cccc(-c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8cccc(-c9ccccc9)c8)cccc76)cccc54)c3)c2)cc1 ; c1ccc(-c2cccc(-c3ccccc3-n3c4ccccc4c4ccc(-n5c6ccccc6c6c([Si](c7ccccc7)(c7ccccc7)c7cccc(-c8ccccc8)c7)cccc65)cc43)c2)cc1]

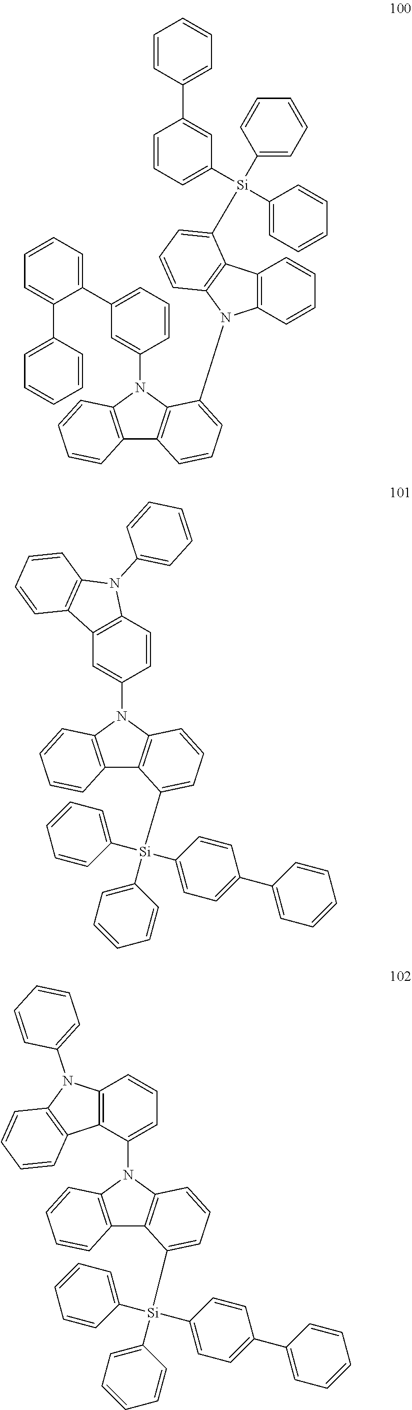

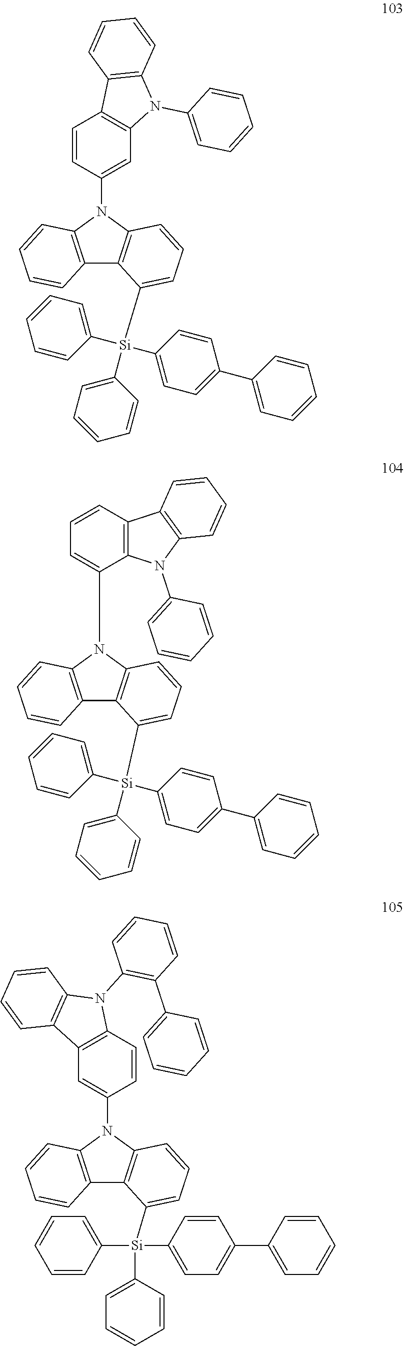

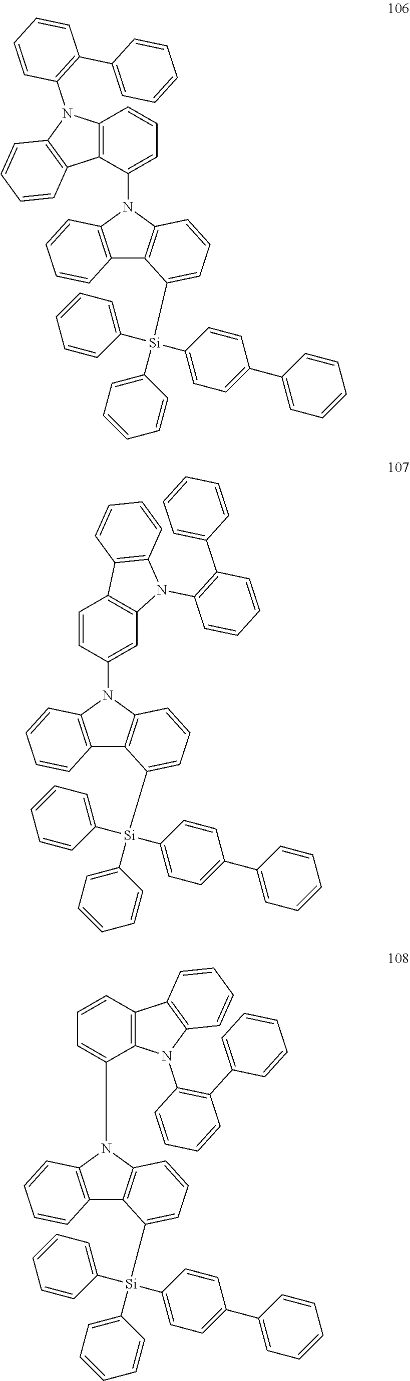 [SMILES: c1ccc(-c2ccc([Si](c3ccccc3)(c3ccccc3)c3cccc4c3c3ccccc3n4-c3cccc4c3c3ccccc3n4-c3ccccc3-c3ccccc3)cc2)cc1 ; c1ccc(-c2ccc([Si](c3ccccc3)(c3ccccc3)c3cccc4c3c3ccccc3n4-c3ccc4c5ccccc5n(-c5ccccc5-c5ccccc5)c4c3)cc2)cc1 ; c1ccc(-c2ccc([Si](c3ccccc3)(c3ccccc3)c3cccc4c3c3ccccc3n4-c3cccc4c5ccccc5n(-c5ccccc5-c5ccccc5)c34)cc2)cc1]

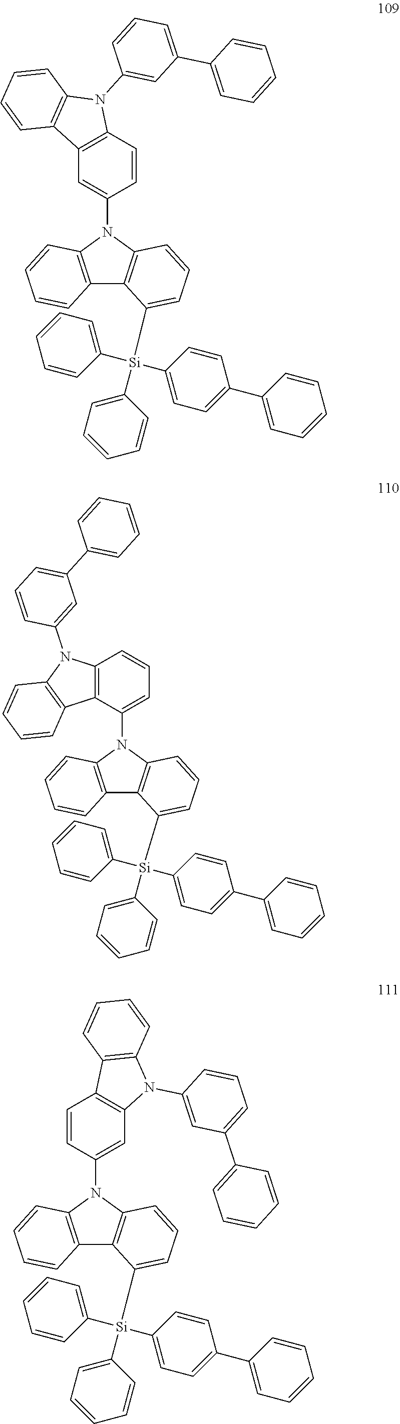

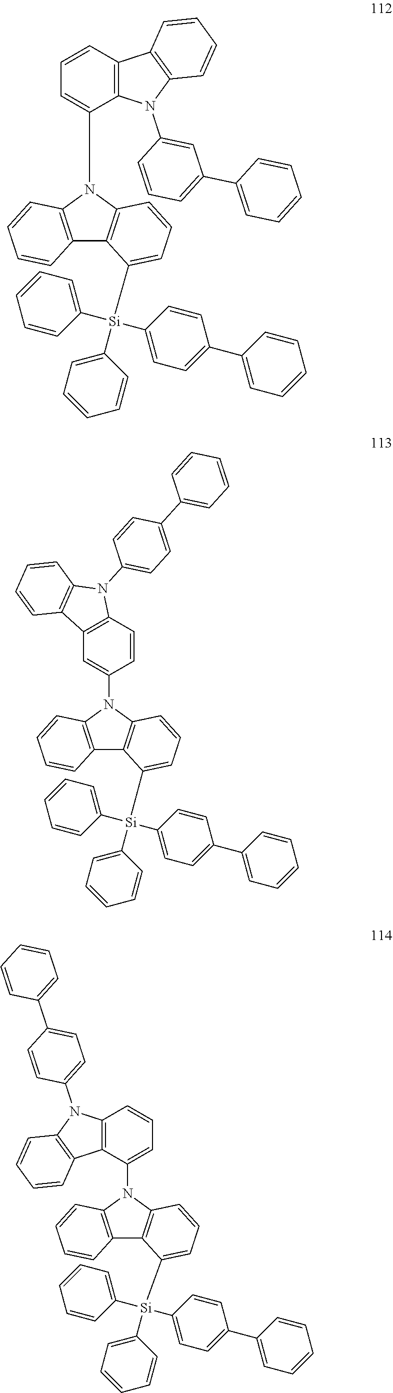 [SMILES: c1ccc(-c2ccc([Si](c3ccccc3)(c3ccccc3)c3cccc4c3c3ccccc3n4-c3cccc4c5ccccc5n(-c5cccc(-c6ccccc6)c5)c34)cc2)cc1 ; c1ccc(-c2ccc(-n3c4ccccc4c4cc(-n5c6ccccc6c6c([Si](c7ccccc7)(c7ccccc7)c7ccc(-c8ccccc8)cc7)cccc65)ccc43)cc2)cc1 ; c1ccc(-c2ccc(-n3c4ccccc4c4c(-n5c6ccccc6c6c([Si](c7ccccc7)(c7ccccc7)c7ccc(-c8ccccc8)cc7)cccc65)cccc43)cc2)cc1]

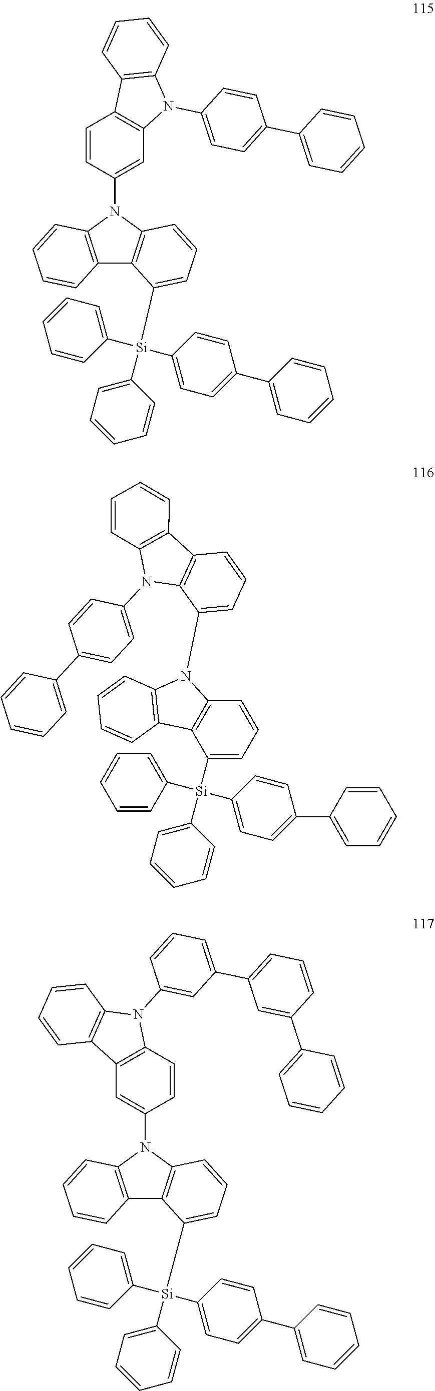

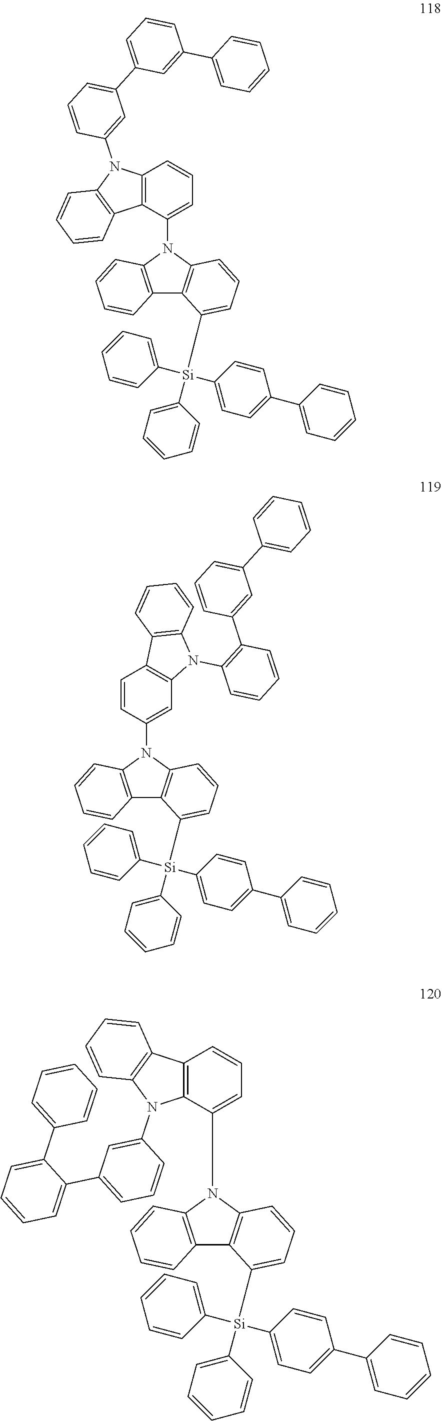 [SMILES: c1ccc(-c2ccc([Si](c3ccccc3)(c3ccccc3)c3cccc4c3c3ccccc3n4-c3cccc4c3c3ccccc3n4-c3cccc(-c4cccc(-c5ccccc5)c4)c3)cc2)cc1 ; c1ccc(-c2ccc([Si](c3ccccc3)(c3ccccc3)c3cccc4c3c3ccccc3n4-c3ccc4c5ccccc5n(-c5ccccc5-c5cccc(-c6ccccc6)c5)c4c3)cc2)cc1 ; c1ccc(-c2ccc([Si](c3ccccc3)(c3ccccc3)c3cccc4c3c3ccccc3n4-c3cccc4c5ccccc5n(-c5cccc(-c6ccccc6-c6ccccc6)c5)c34)cc2)cc1]

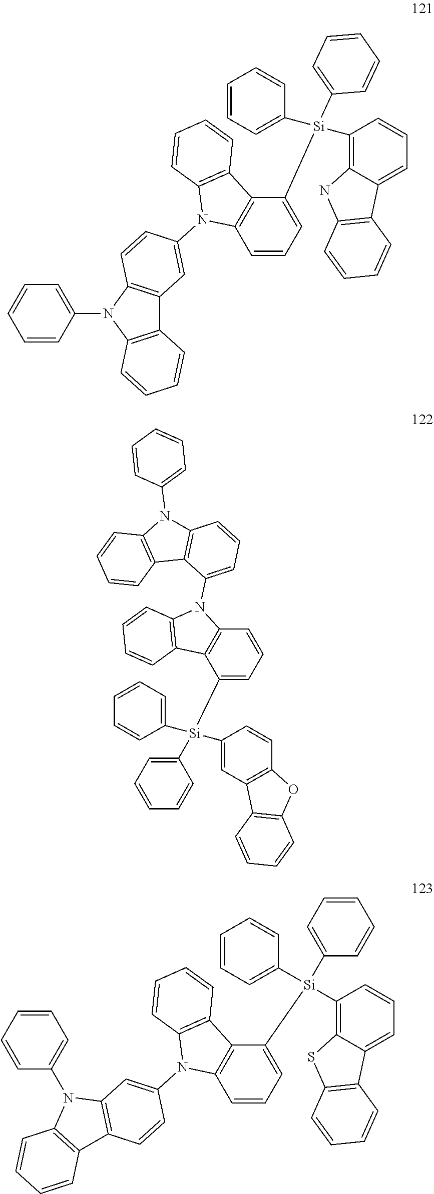

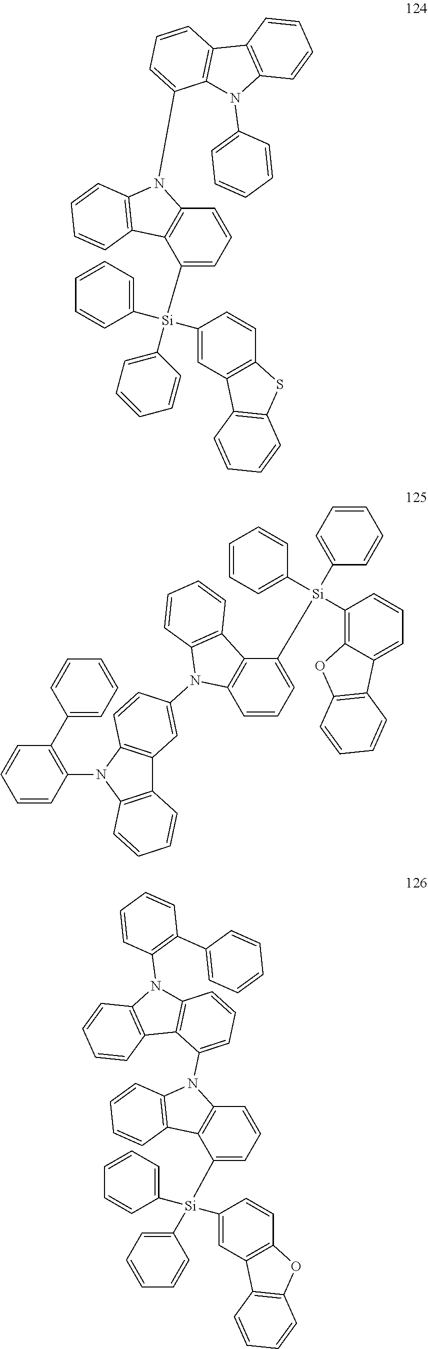

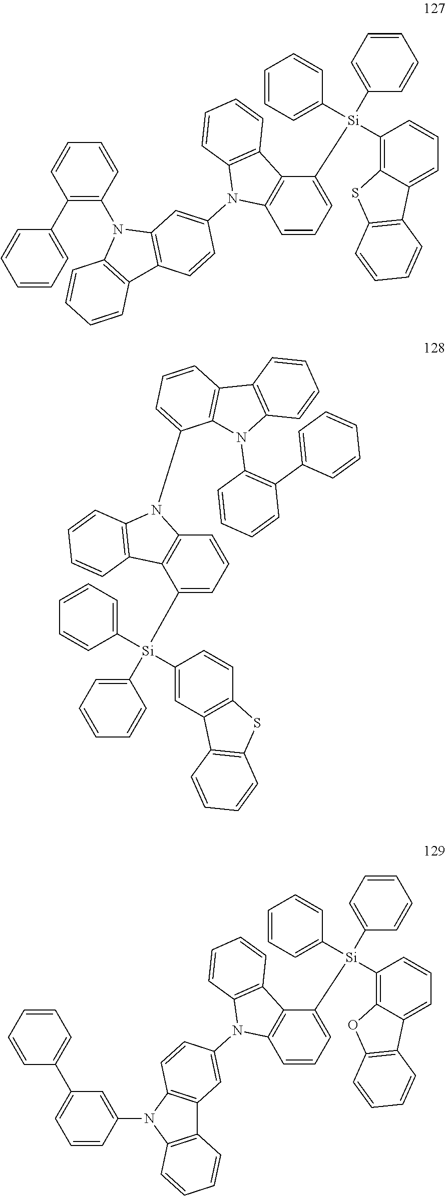 [SMILES: c1ccc(-c2ccccc2-n2c3ccccc3c3ccc(-n4c5ccccc5c5c([Si](c6ccccc6)(c6ccccc6)c6cccc7c6sc6ccccc67)cccc54)cc32)cc1 ; c1ccc(-c2ccccc2-n2c3ccccc3c3cccc(-n4c5ccccc5c5c([Si](c6ccccc6)(c6ccccc6)c6ccc7sc8ccccc8c7c6)cccc54)c32)cc1 ; c1ccc(-c2cccc(-n3c4ccccc4c4cc(-n5c6ccccc6c6c([Si](c7ccccc7)(c7ccccc7)c7cccc8c7oc7ccccc78)cccc65)ccc43)c2)cc1]

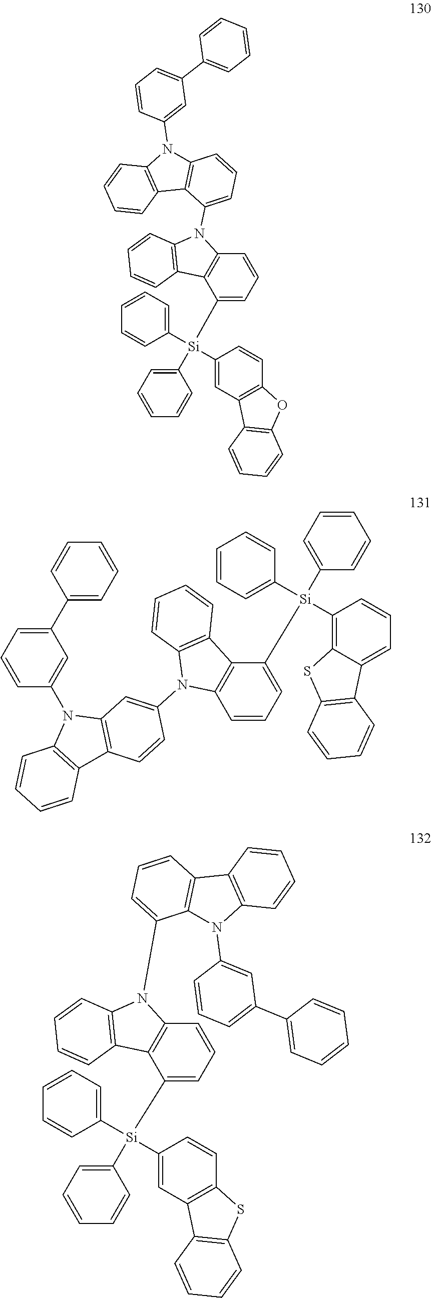 [SMILES: c1ccc(-c2cccc(-n3c4ccccc4c4c(-n5c6ccccc6c6c([Si](c7ccccc7)(c7ccccc7)c7ccc8oc9ccccc9c8c7)cccc65)cccc43)c2)cc1 ; c1ccc(-c2cccc(-n3c4ccccc4c4ccc(-n5c6ccccc6c6c([Si](c7ccccc7)(c7ccccc7)c7cccc8c7sc7ccccc78)cccc65)cc43)c2)cc1 ; c1ccc(-c2cccc(-n3c4ccccc4c4cccc(-n5c6ccccc6c6c([Si](c7ccccc7)(c7ccccc7)c7ccc8sc9ccccc9c8c7)cccc65)c43)c2)cc1]

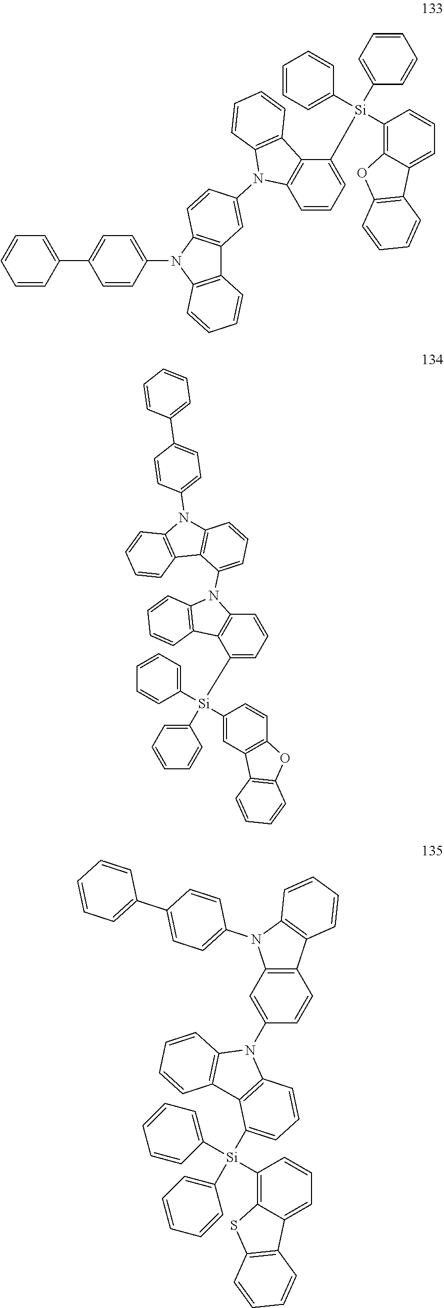 [SMILES: c1ccc(-c2ccc(-n3c4ccccc4c4cc(-n5c6ccccc6c6c([Si](c7ccccc7)(c7ccccc7)c7cccc8c7oc7ccccc78)cccc65)ccc43)cc2)cc1 ; c1ccc(-c2ccc(-n3c4ccccc4c4c(-n5c6ccccc6c6c([Si](c7ccccc7)(c7ccccc7)c7ccc8oc9ccccc9c8c7)cccc65)cccc43)cc2)cc1 ; c1ccc(-c2ccc(-n3c4ccccc4c4ccc(-n5c6ccccc6c6c([Si](c7ccccc7)(c7ccccc7)c7cccc8c7sc7ccccc78)cccc65)cc43)cc2)cc1]

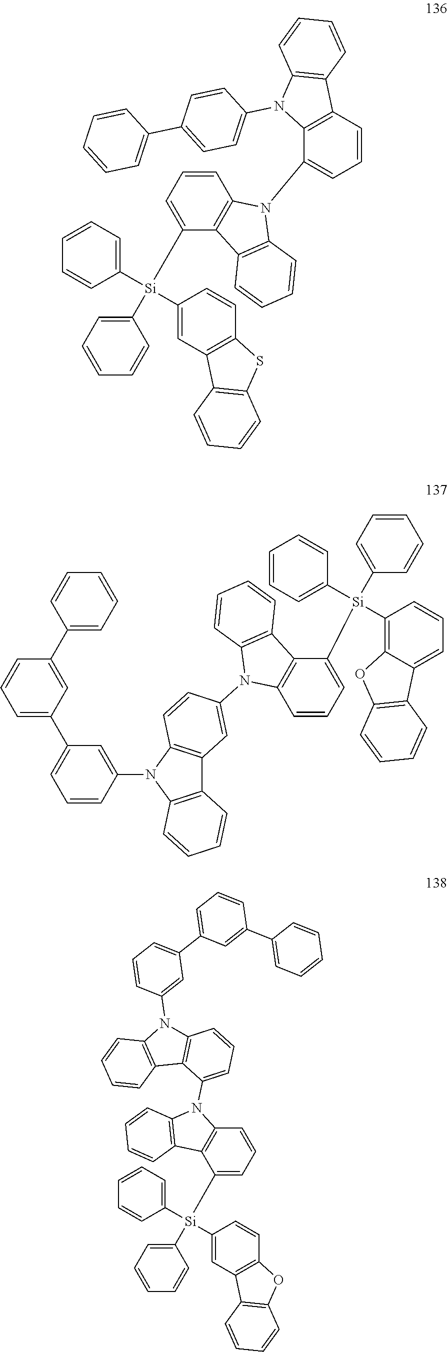

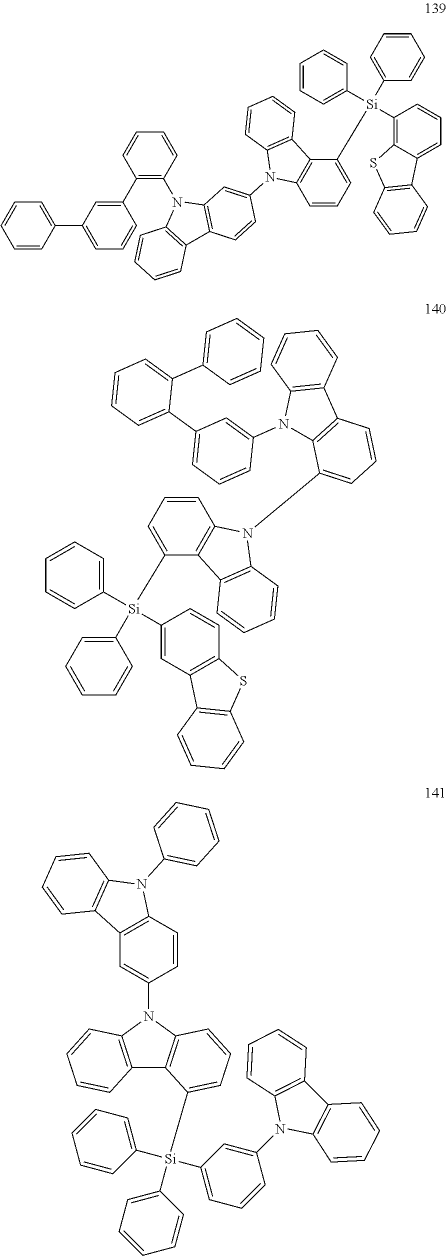

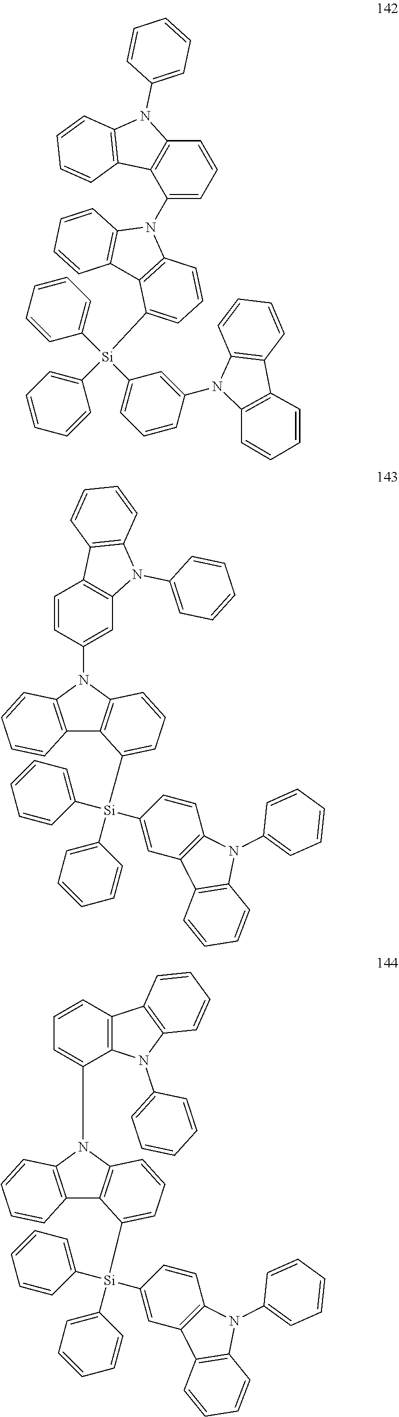

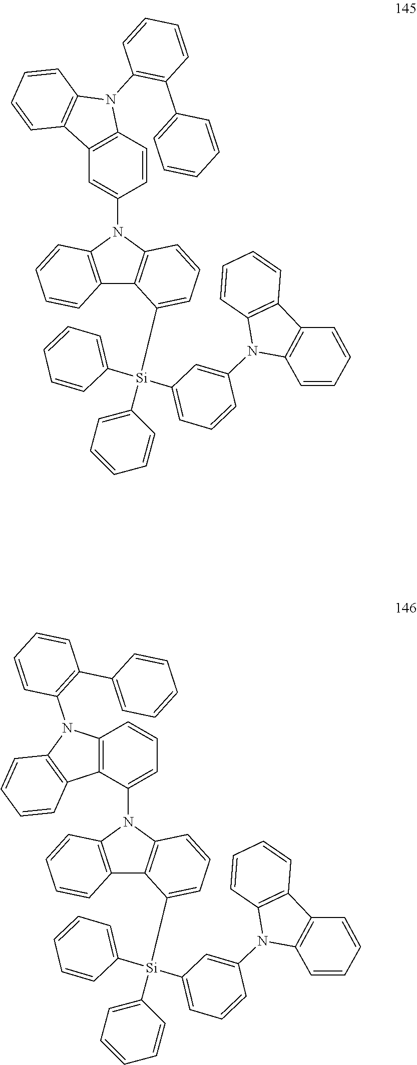 [SMILES: c1ccc(-c2ccccc2-n2c3ccccc3c3cc(-n4c5ccccc5c5c([Si](c6ccccc6)(c6ccccc6)c6cccc(-n7c8ccccc8c8ccccc87)c6)cccc54)ccc32)cc1 ; c1ccc(-c2ccccc2-n2c3ccccc3c3c(-n4c5ccccc5c5c([Si](c6ccccc6)(c6ccccc6)c6cccc(-n7c8ccccc8c8ccccc87)c6)cccc54)cccc32)cc1]

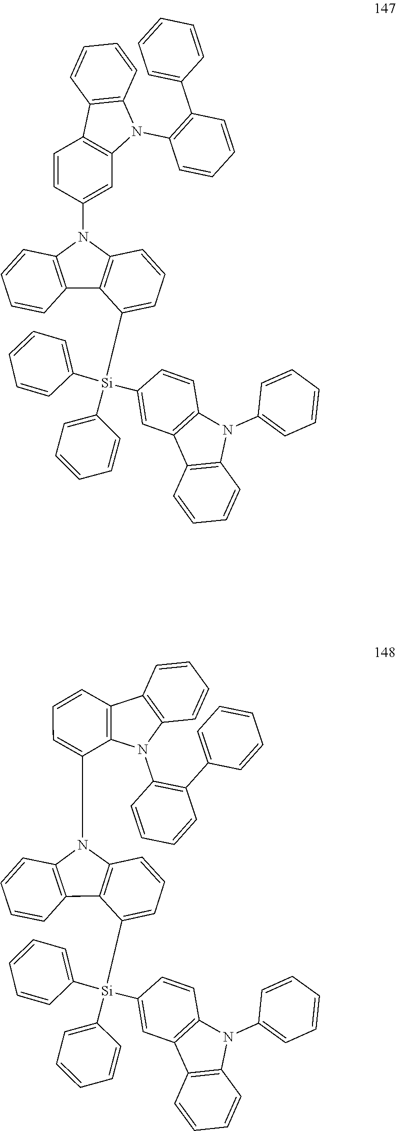 [SMILES: c1ccc(-c2ccccc2-n2c3ccccc3c3ccc(-n4c5ccccc5c5c([Si](c6ccccc6)(c6ccccc6)c6ccc7c(c6)c6ccccc6n7-c6ccccc6)cccc54)cc32)cc1 ; c1ccc(-c2ccccc2-n2c3ccccc3c3cccc(-n4c5ccccc5c5c([Si](c6ccccc6)(c6ccccc6)c6ccc7c(c6)c6ccccc6n7-c6ccccc6)cccc54)c32)cc1]

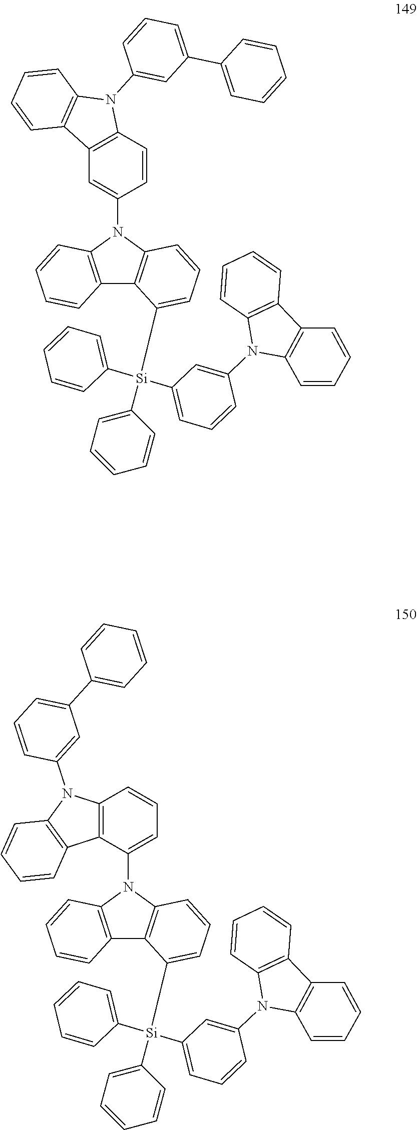 [SMILES: c1ccc(-c2cccc(-n3c4ccccc4c4cc(-n5c6ccccc6c6c([Si](c7ccccc7)(c7ccccc7)c7cccc(-n8c9ccccc9c9ccccc98)c7)cccc65)ccc43)c2)cc1 ; c1ccc(-c2cccc(-n3c4ccccc4c4c(-n5c6ccccc6c6c([Si](c7ccccc7)(c7ccccc7)c7cccc(-n8c9ccccc9c9ccccc98)c7)cccc65)cccc43)c2)cc1]

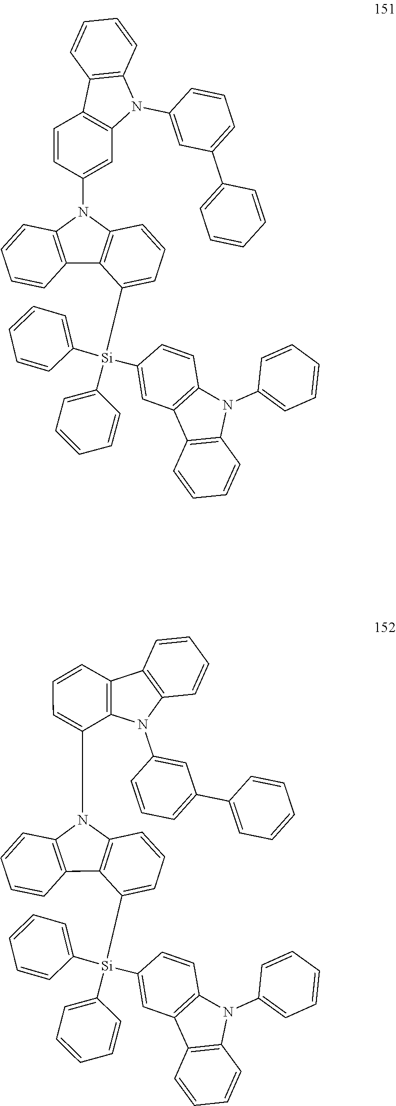 [SMILES: c1ccc(-c2cccc(-n3c4ccccc4c4ccc(-n5c6ccccc6c6c([Si](c7ccccc7)(c7ccccc7)c7ccc8c(c7)c7ccccc7n8-c7ccccc7)cccc65)cc43)c2)cc1 ; c1ccc(-c2cccc(-n3c4ccccc4c4cccc(-n5c6ccccc6c6c([Si](c7ccccc7)(c7ccccc7)c7ccc8c(c7)c7ccccc7n8-c7ccccc7)cccc65)c43)c2)cc1]

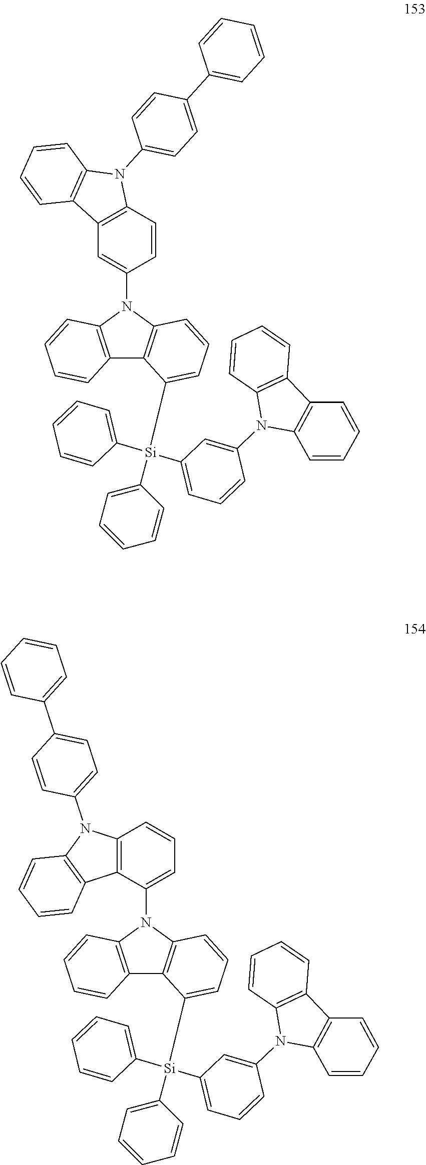 [SMILES: c1ccc(-c2ccc(-n3c4ccccc4c4cc(-n5c6ccccc6c6c([Si](c7ccccc7)(c7ccccc7)c7cccc(-n8c9ccccc9c9ccccc98)c7)cccc65)ccc43)cc2)cc1 ; c1ccc(-c2ccc(-n3c4ccccc4c4c(-n5c6ccccc6c6c([Si](c7ccccc7)(c7ccccc7)c7cccc(-n8c9ccccc9c9ccccc98)c7)cccc65)cccc43)cc2)cc1]

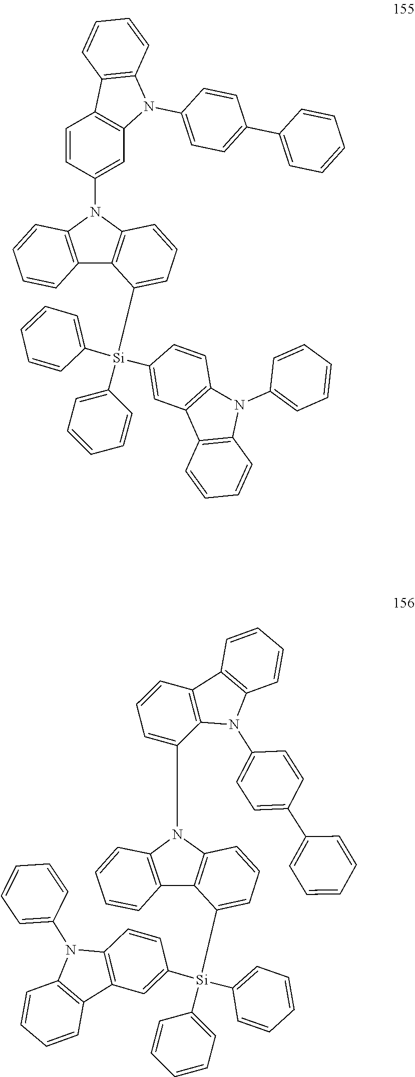 [SMILES: c1ccc(-c2ccc(-n3c4ccccc4c4ccc(-n5c6ccccc6c6c([Si](c7ccccc7)(c7ccccc7)c7ccc8c(c7)c7ccccc7n8-c7ccccc7)cccc65)cc43)cc2)cc1 ; c1ccc(-c2ccc(-n3c4ccccc4c4cccc(-n5c6ccccc6c6c([Si](c7ccccc7)(c7ccccc7)c7ccc8c(c7)c7ccccc7n8-c7ccccc7)cccc65)c43)cc2)cc1]

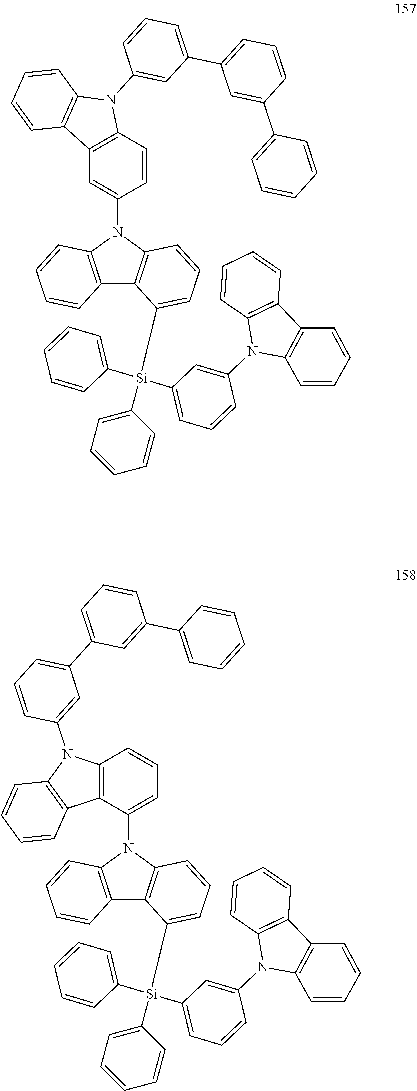 [SMILES: c1ccc(-c2cccc(-c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)c3)c2)cc1]

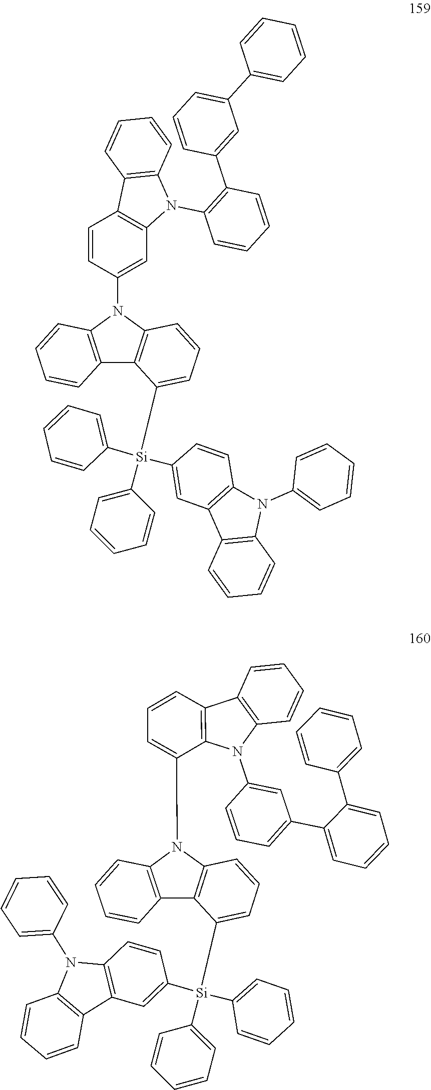 [SMILES: c1ccc(-c2cccc(-c3ccccc3-n3c4ccccc4c4ccc(-n5c6ccccc6c6c([Si](c7ccccc7)(c7ccccc7)c7ccc8c(c7)c7ccccc7n8-c7ccccc7)cccc65)cc43)c2)cc1 ; c1ccc(-c2ccccc2-c2cccc(-n3c4ccccc4c4cccc(-n5c6ccccc6c6c([Si](c7ccccc7)(c7ccccc7)c7ccc8c(c7)c7ccccc7n8-c7ccccc7)cccc65)c43)c2)cc1]

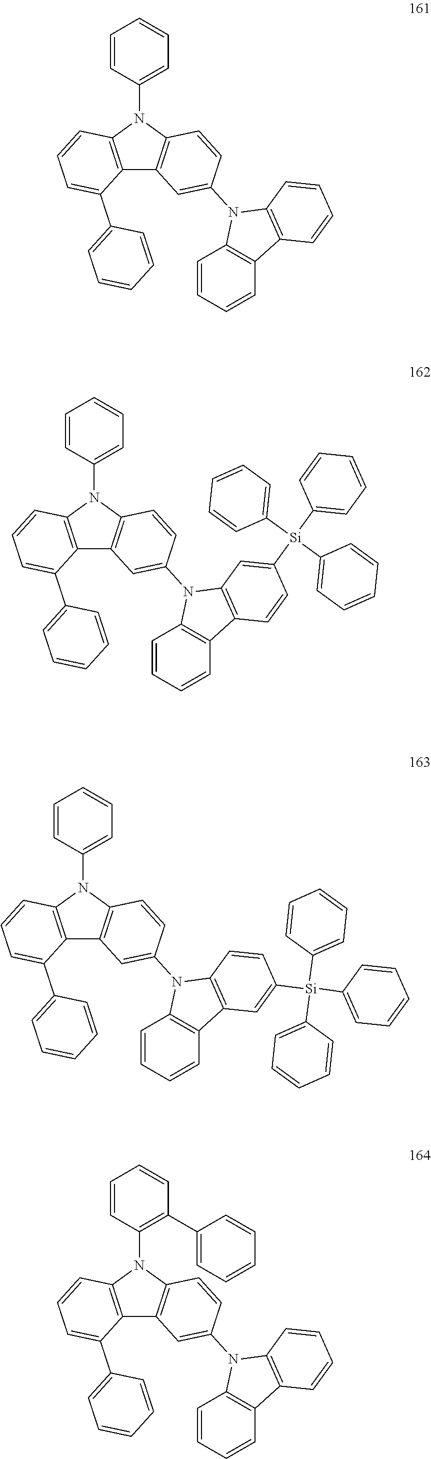 [SMILES: c1ccc(-c2cccc3c2c2cc(-n4c5ccccc5c5ccccc54)ccc2n3-c2ccccc2)cc1 ; c1ccc(-c2cccc3c2c2cc(-n4c5ccccc5c5ccc([Si](c6ccccc6)(c6ccccc6)c6ccccc6)cc54)ccc2n3-c2ccccc2)cc1 ; c1ccc(-c2cccc3c2c2cc(-n4c5ccccc5c5cc([Si](c6ccccc6)(c6ccccc6)c6ccccc6)ccc54)ccc2n3-c2ccccc2)cc1 ; c1ccc(-c2ccccc2-n2c3ccc(-n4c5ccccc5c5ccccc54)cc3c3c(-c4ccccc4)cccc32)cc1]

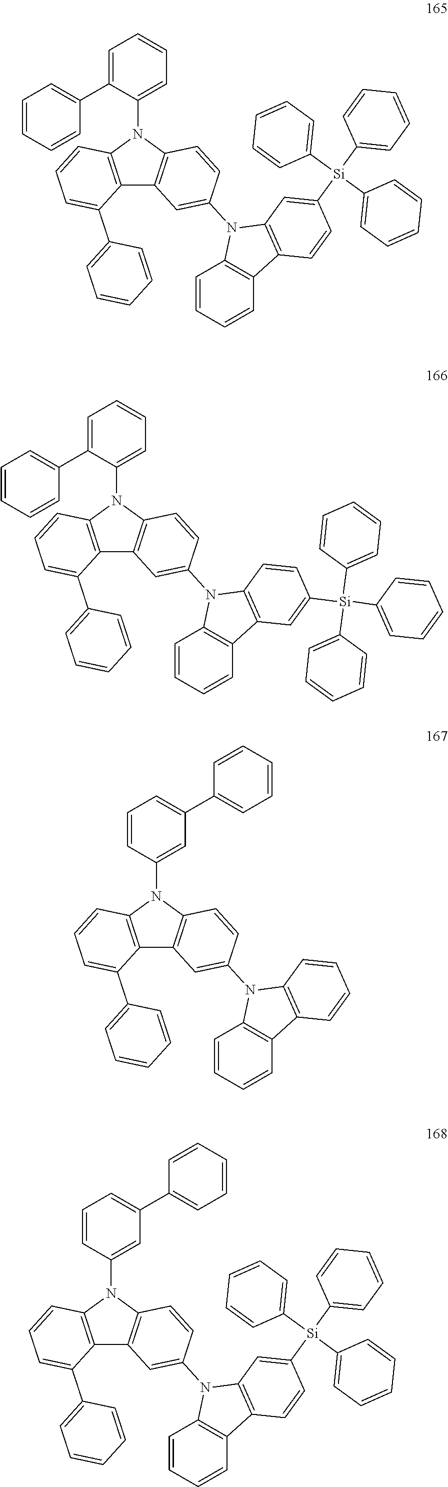 [SMILES: c1ccc(-c2ccccc2-n2c3ccc(-n4c5ccccc5c5ccc([Si](c6ccccc6)(c6ccccc6)c6ccccc6)cc54)cc3c3c(-c4ccccc4)cccc32)cc1 ; c1ccc(-c2ccccc2-n2c3ccc(-n4c5ccccc5c5cc([Si](c6ccccc6)(c6ccccc6)c6ccccc6)ccc54)cc3c3c(-c4ccccc4)cccc32)cc1 ; c1ccc(-c2cccc(-n3c4ccc(-n5c6ccccc6c6ccccc65)cc4c4c(-c5ccccc5)cccc43)c2)cc1 ; c1ccc(-c2cccc(-n3c4ccc(-n5c6ccccc6c6ccc([Si](c7ccccc7)(c7ccccc7)c7ccccc7)cc65)cc4c4c(-c5ccccc5)cccc43)c2)cc1]

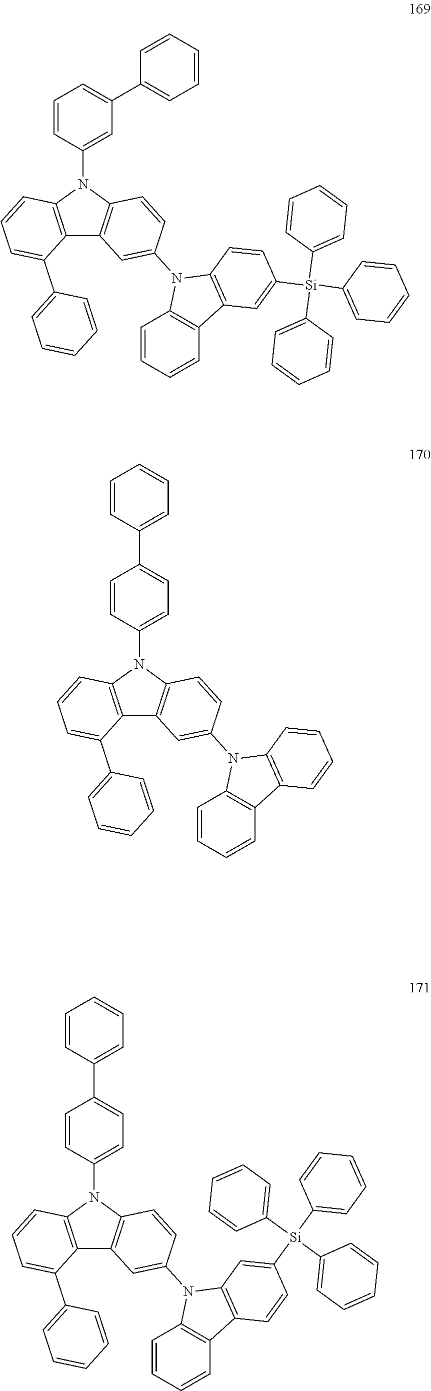 [SMILES: c1ccc(-c2cccc(-n3c4ccc(-n5c6ccccc6c6cc([Si](c7ccccc7)(c7ccccc7)c7ccccc7)ccc65)cc4c4c(-c5ccccc5)cccc43)c2)cc1 ; c1ccc(-c2ccc(-n3c4ccc(-n5c6ccccc6c6ccccc65)cc4c4c(-c5ccccc5)cccc43)cc2)cc1 ; c1ccc(-c2ccc(-n3c4ccc(-n5c6ccccc6c6ccc([Si](c7ccccc7)(c7ccccc7)c7ccccc7)cc65)cc4c4c(-c5ccccc5)cccc43)cc2)cc1]

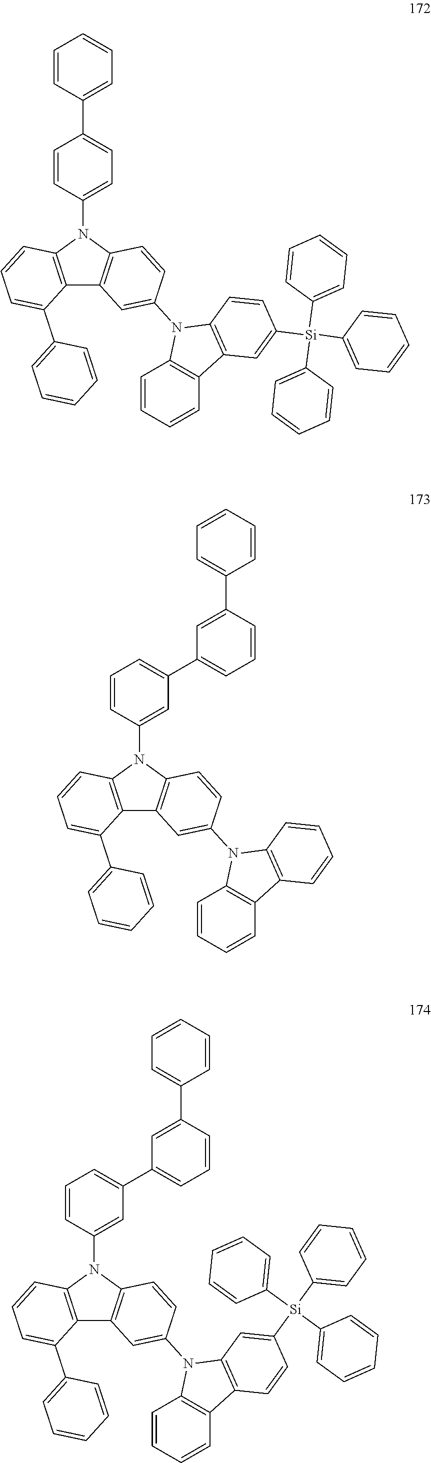 [SMILES: c1ccc(-c2ccc(-n3c4ccc(-n5c6ccccc6c6cc([Si](c7ccccc7)(c7ccccc7)c7ccccc7)ccc65)cc4c4c(-c5ccccc5)cccc43)cc2)cc1 ; c1ccc(-c2cccc(-c3cccc(-n4c5ccc(-n6c7ccccc7c7ccccc76)cc5c5c(-c6ccccc6)cccc54)c3)c2)cc1 ; c1ccc(-c2cccc(-c3cccc(-n4c5ccc(-n6c7ccccc7c7ccc([Si](c8ccccc8)(c8ccccc8)c8ccccc8)cc76)cc5c5c(-c6ccccc6)cccc54)c3)c2)cc1]

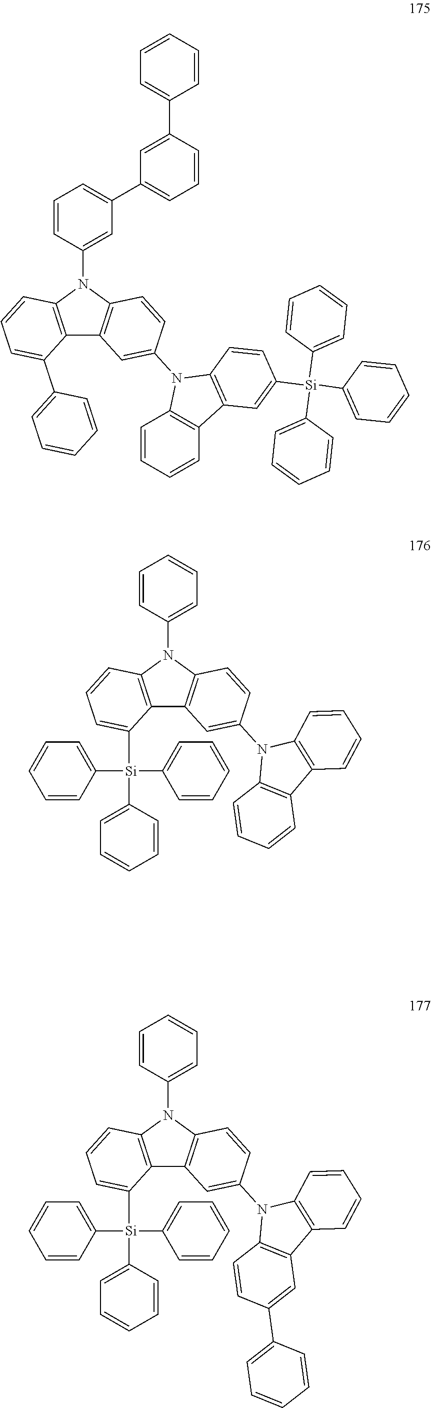 [SMILES: c1ccc(-c2cccc(-c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1 ; c1ccc(-n2c3ccc(-n4c5ccccc5c5ccccc54)cc3c3c([Si](c4ccccc4)(c4ccccc4)c4ccccc4)cccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2ccccc2)cc1]

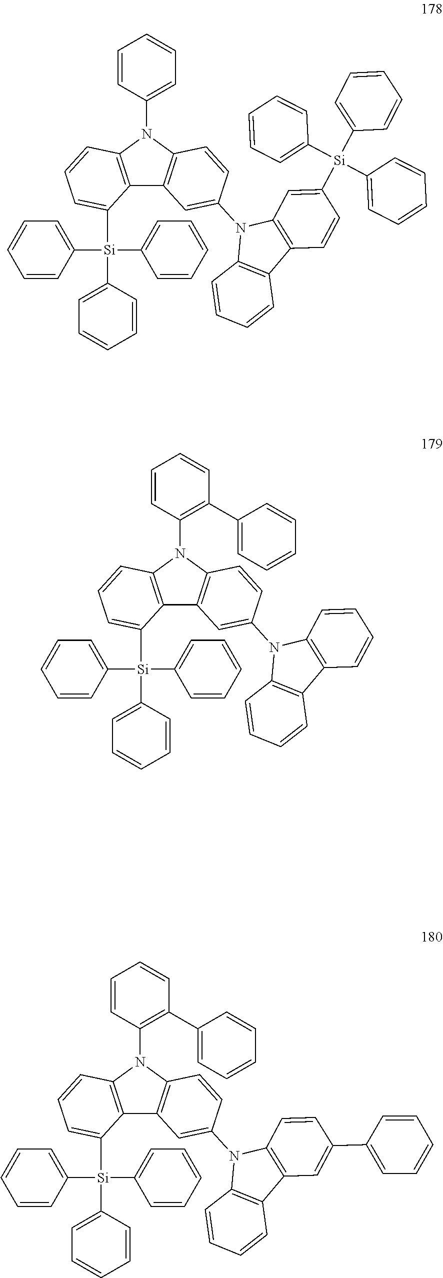 [SMILES: c1ccc(-n2c3ccc(-n4c5ccccc5c5ccc([Si](c6ccccc6)(c6ccccc6)c6ccccc6)cc54)cc3c3c([Si](c4ccccc4)(c4ccccc4)c4ccccc4)cccc32)cc1 ; c1ccc(-c2ccccc2-n2c3ccc(-n4c5ccccc5c5ccccc54)cc3c3c([Si](c4ccccc4)(c4ccccc4)c4ccccc4)cccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2ccccc2-c2ccccc2)cc1]

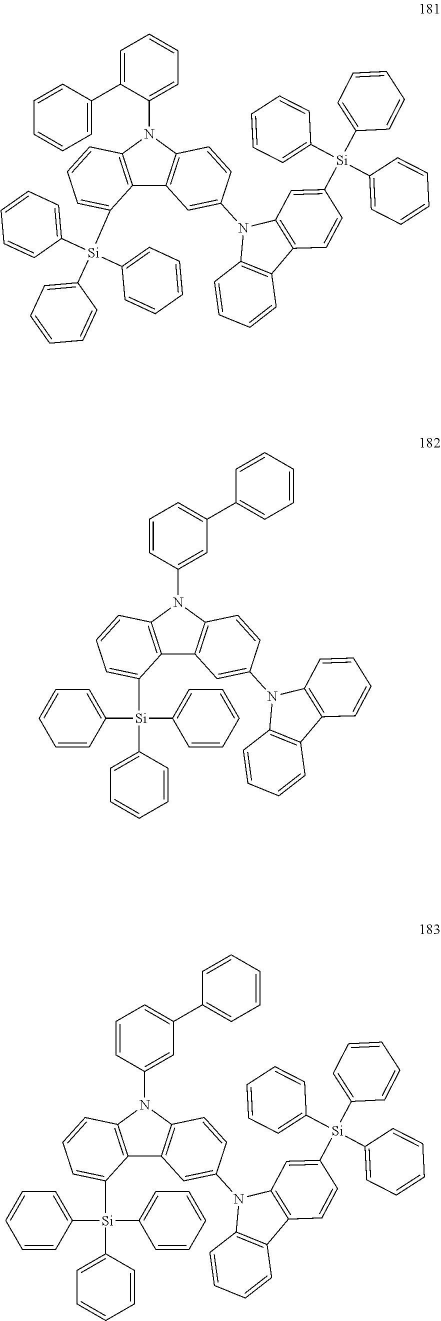 [SMILES: c1ccc(-c2ccccc2-n2c3ccc(-n4c5ccccc5c5ccc([Si](c6ccccc6)(c6ccccc6)c6ccccc6)cc54)cc3c3c([Si](c4ccccc4)(c4ccccc4)c4ccccc4)cccc32)cc1 ; c1ccc(-c2cccc(-n3c4ccc(-n5c6ccccc6c6ccccc65)cc4c4c([Si](c5ccccc5)(c5ccccc5)c5ccccc5)cccc43)c2)cc1 ; c1ccc(-c2cccc(-n3c4ccc(-n5c6ccccc6c6ccc([Si](c7ccccc7)(c7ccccc7)c7ccccc7)cc65)cc4c4c([Si](c5ccccc5)(c5ccccc5)c5ccccc5)cccc43)c2)cc1]

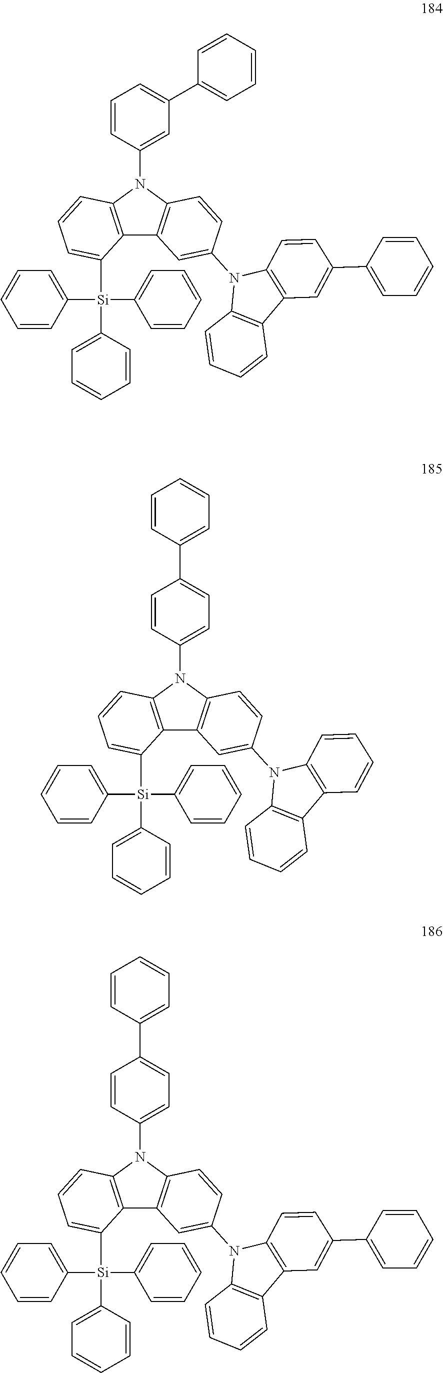 [SMILES: c1ccc(-c2cccc(-n3c4ccc(-n5c6ccccc6c6cc(-c7ccccc7)ccc65)cc4c4c([Si](c5ccccc5)(c5ccccc5)c5ccccc5)cccc43)c2)cc1 ; c1ccc(-c2ccc(-n3c4ccc(-n5c6ccccc6c6ccccc65)cc4c4c([Si](c5ccccc5)(c5ccccc5)c5ccccc5)cccc43)cc2)cc1 ; c1ccc(-c2ccc(-n3c4ccc(-n5c6ccccc6c6cc(-c7ccccc7)ccc65)cc4c4c([Si](c5ccccc5)(c5ccccc5)c5ccccc5)cccc43)cc2)cc1]

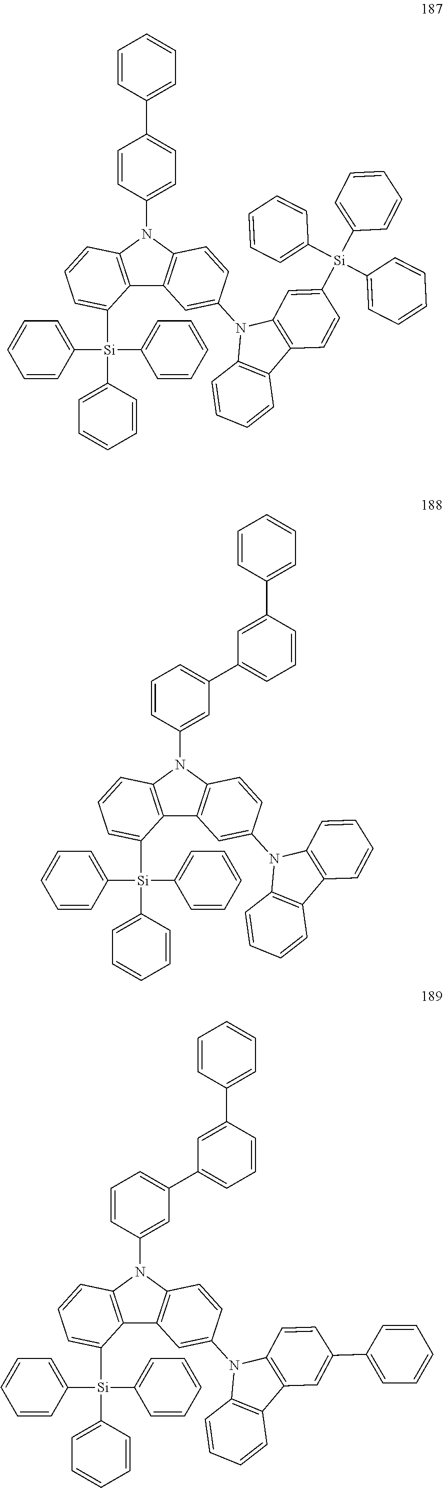 [SMILES: c1ccc(-c2ccc(-n3c4ccc(-n5c6ccccc6c6ccc([Si](c7ccccc7)(c7ccccc7)c7ccccc7)cc65)cc4c4c([Si](c5ccccc5)(c5ccccc5)c5ccccc5)cccc43)cc2)cc1 ; c1ccc(-c2cccc(-c3cccc(-n4c5ccc(-n6c7ccccc7c7ccccc76)cc5c5c([Si](c6ccccc6)(c6ccccc6)c6ccccc6)cccc54)c3)c2)cc1 ; c1ccc(-c2cccc(-c3cccc(-n4c5ccc(-n6c7ccccc7c7cc(-c8ccccc8)ccc76)cc5c5c([Si](c6ccccc6)(c6ccccc6)c6ccccc6)cccc54)c3)c2)cc1]

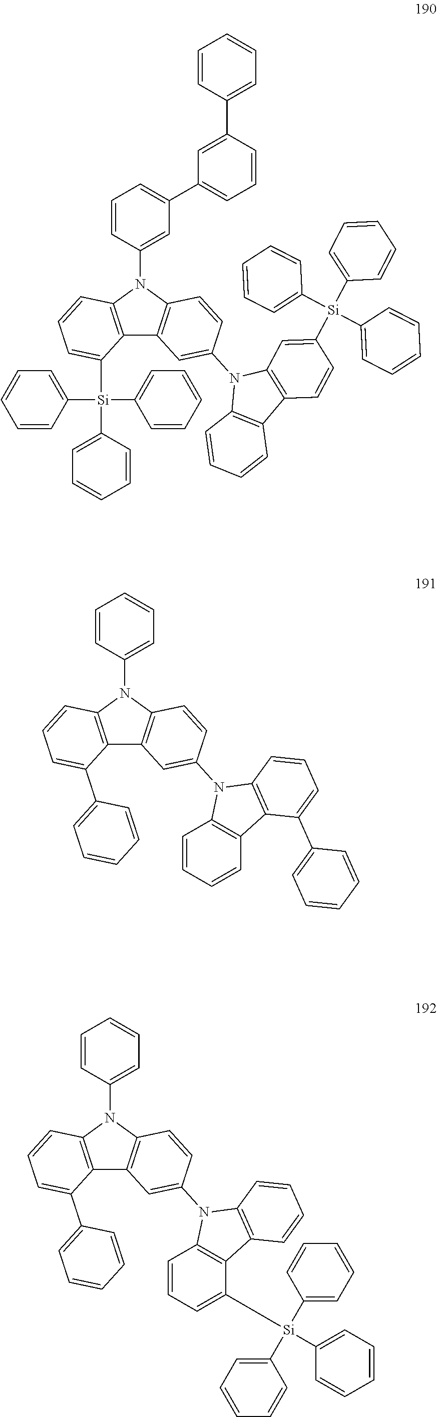 [SMILES: c1ccc(-c2cccc(-c3cccc(-n4c5ccc(-n6c7ccccc7c7ccc([Si](c8ccccc8)(c8ccccc8)c8ccccc8)cc76)cc5c5c([Si](c6ccccc6)(c6ccccc6)c6ccccc6)cccc54)c3)c2)cc1 ; c1ccc(-c2cccc3c2c2ccccc2n3-c2ccc3c(c2)c2c(-c4ccccc4)cccc2n3-c2ccccc2)cc1 ; c1ccc(-c2cccc3c2c2cc(-n4c5ccccc5c5c([Si](c6ccccc6)(c6ccccc6)c6ccccc6)cccc54)ccc2n3-c2ccccc2)cc1]

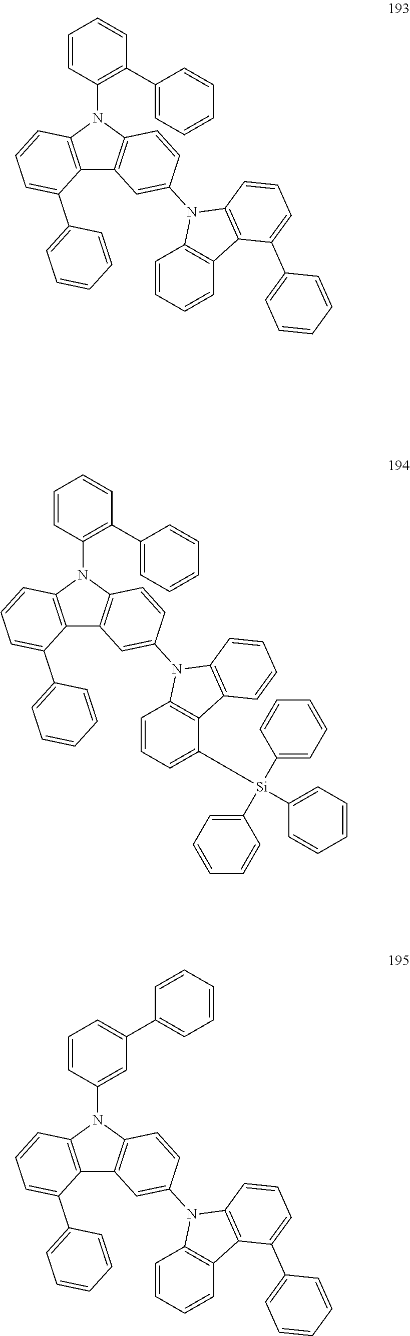 [SMILES: c1ccc(-c2ccccc2-n2c3ccc(-n4c5ccccc5c5c(-c6ccccc6)cccc54)cc3c3c(-c4ccccc4)cccc32)cc1 ; c1ccc(-c2ccccc2-n2c3ccc(-n4c5ccccc5c5c([Si](c6ccccc6)(c6ccccc6)c6ccccc6)cccc54)cc3c3c(-c4ccccc4)cccc32)cc1 ; c1ccc(-c2cccc(-n3c4ccc(-n5c6ccccc6c6c(-c7ccccc7)cccc65)cc4c4c(-c5ccccc5)cccc43)c2)cc1]

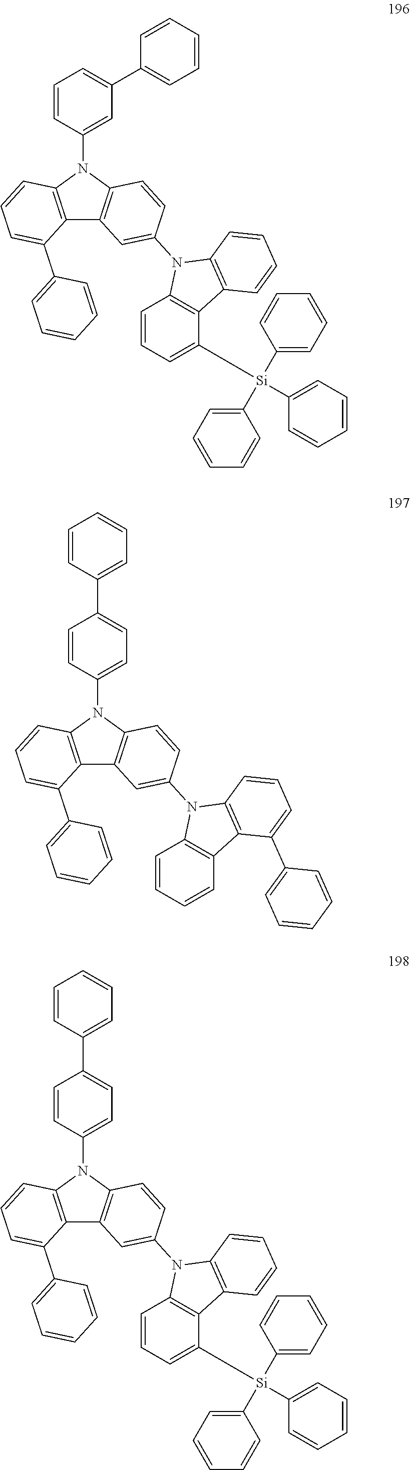 [SMILES: c1ccc(-c2cccc(-n3c4ccc(-n5c6ccccc6c6c([Si](c7ccccc7)(c7ccccc7)c7ccccc7)cccc65)cc4c4c(-c5ccccc5)cccc43)c2)cc1 ; c1ccc(-c2ccc(-n3c4ccc(-n5c6ccccc6c6c(-c7ccccc7)cccc65)cc4c4c(-c5ccccc5)cccc43)cc2)cc1 ; c1ccc(-c2ccc(-n3c4ccc(-n5c6ccccc6c6c([Si](c7ccccc7)(c7ccccc7)c7ccccc7)cccc65)cc4c4c(-c5ccccc5)cccc43)cc2)cc1]

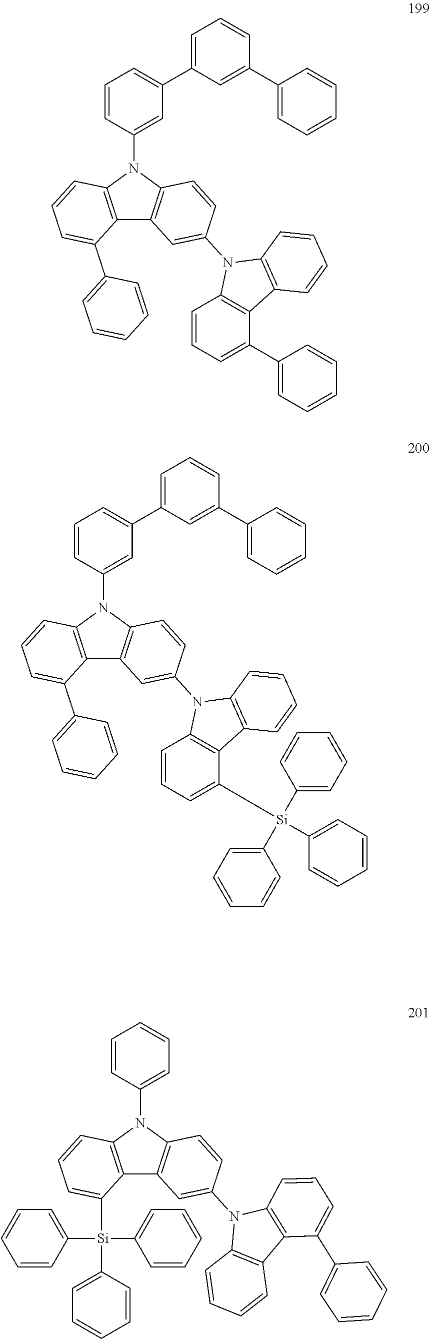

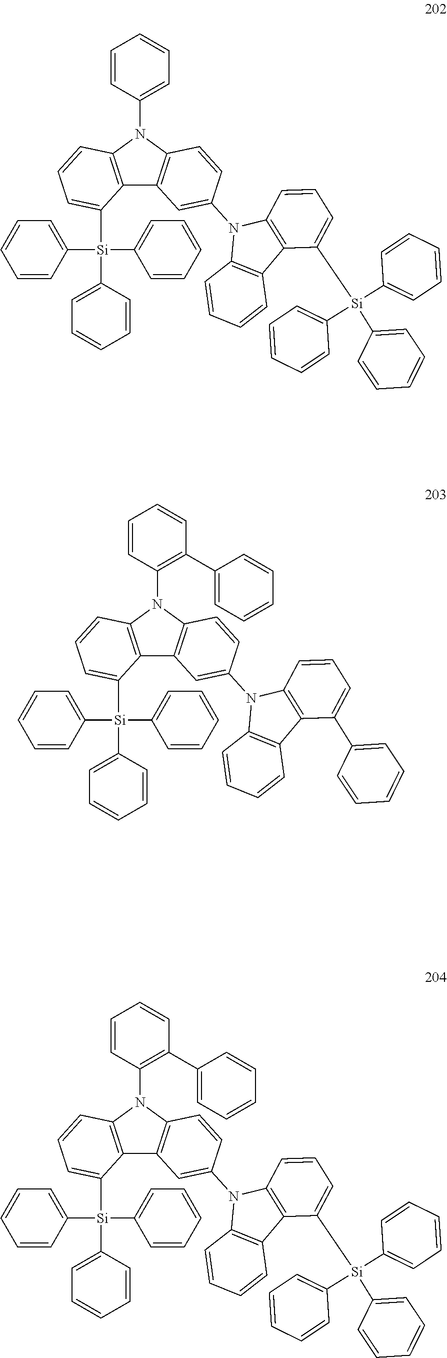

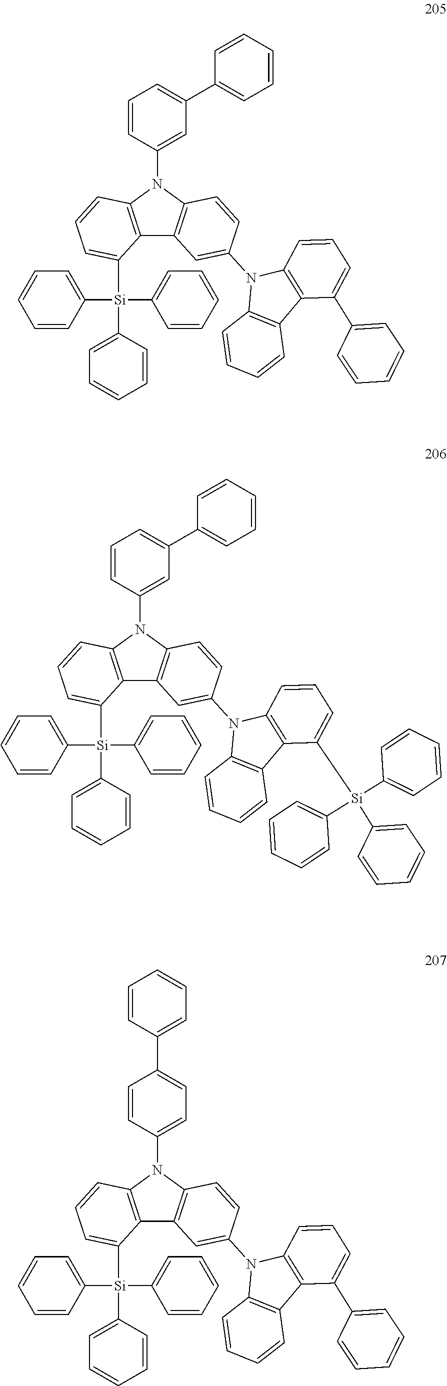 [SMILES: c1ccc(-c2cccc(-n3c4ccc(-n5c6ccccc6c6c(-c7ccccc7)cccc65)cc4c4c([Si](c5ccccc5)(c5ccccc5)c5ccccc5)cccc43)c2)cc1 ; c1ccc(-c2cccc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)c2)cc1 ; c1ccc(-c2ccc(-n3c4ccc(-n5c6ccccc6c6c(-c7ccccc7)cccc65)cc4c4c([Si](c5ccccc5)(c5ccccc5)c5ccccc5)cccc43)cc2)cc1]

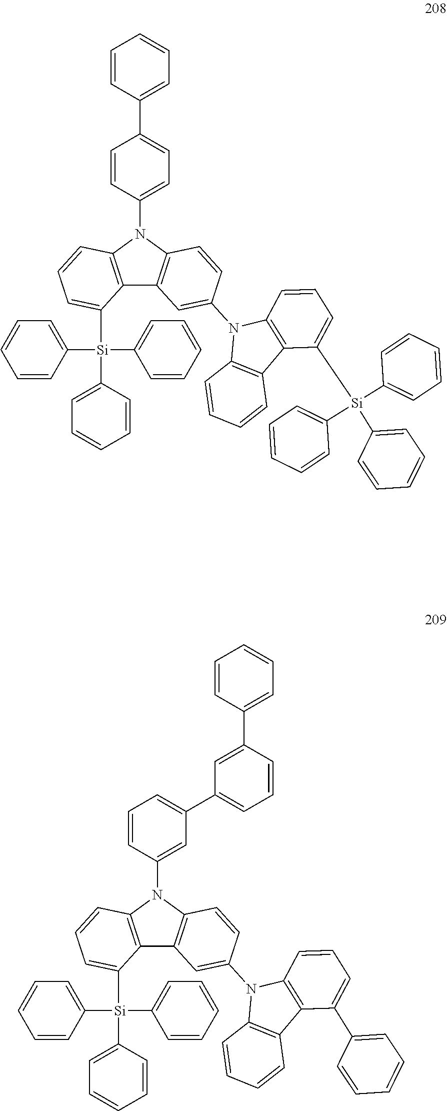 [SMILES: c1ccc(-c2ccc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)cc2)cc1 ; c1ccc(-c2cccc(-c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1]

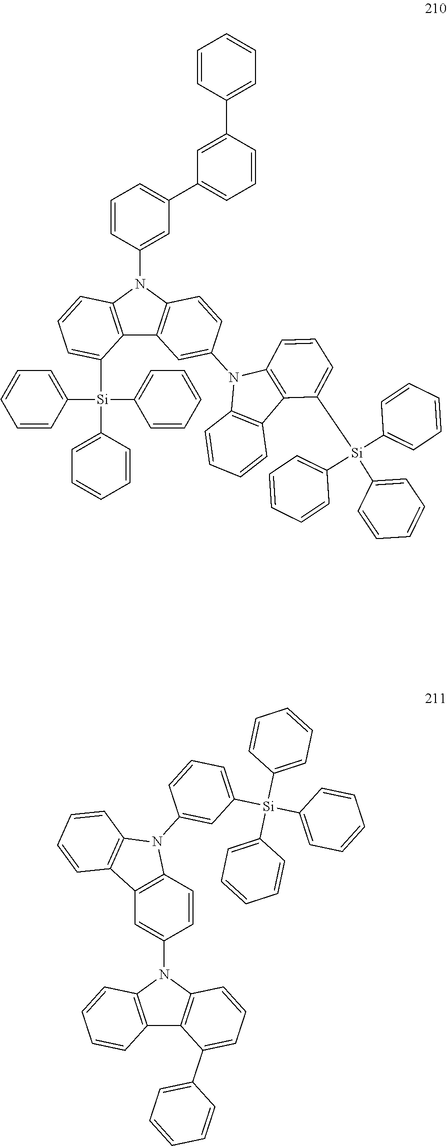 [SMILES: c1ccc(-c2cccc(-c3cccc(-n4c5ccc(-n6c7ccccc7c7c([Si](c8ccccc8)(c8ccccc8)c8ccccc8)cccc76)cc5c5c([Si](c6ccccc6)(c6ccccc6)c6ccccc6)cccc54)c3)c2)cc1 ; c1ccc(-c2cccc3c2c2ccccc2n3-c2ccc3c(c2)c2ccccc2n3-c2cccc([Si](c3ccccc3)(c3ccccc3)c3ccccc3)c2)cc1]

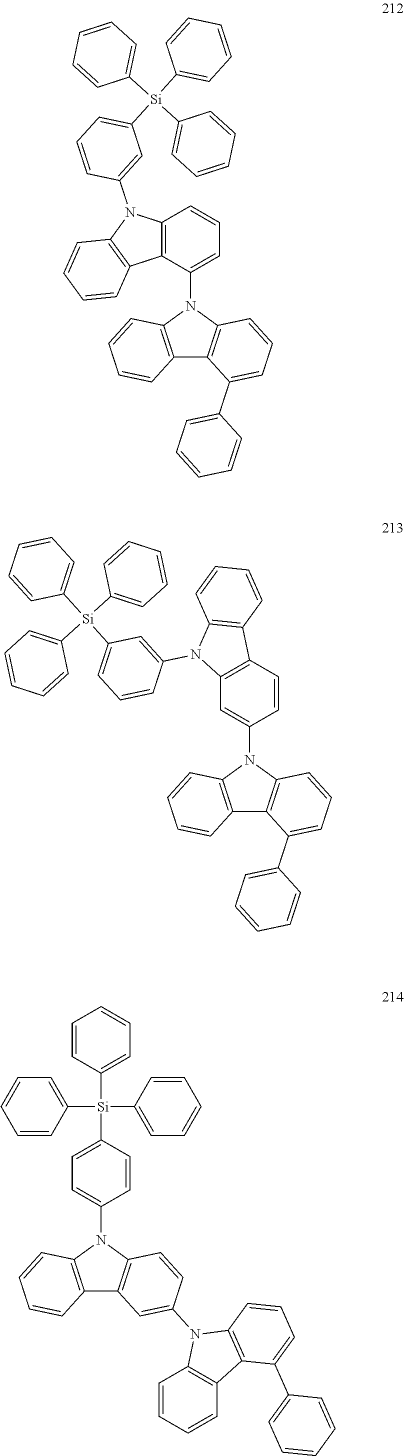 [SMILES: c1ccc(-c2cccc3c2c2ccccc2n3-c2cccc3c2c2ccccc2n3-c2cccc([Si](c3ccccc3)(c3ccccc3)c3ccccc3)c2)cc1 ; c1ccc(-c2cccc3c2c2ccccc2n3-c2ccc3c4ccccc4n(-c4cccc([Si](c5ccccc5)(c5ccccc5)c5ccccc5)c4)c3c2)cc1 ; c1ccc(-c2cccc3c2c2ccccc2n3-c2ccc3c(c2)c2ccccc2n3-c2ccc([Si](c3ccccc3)(c3ccccc3)c3ccccc3)cc2)cc1]

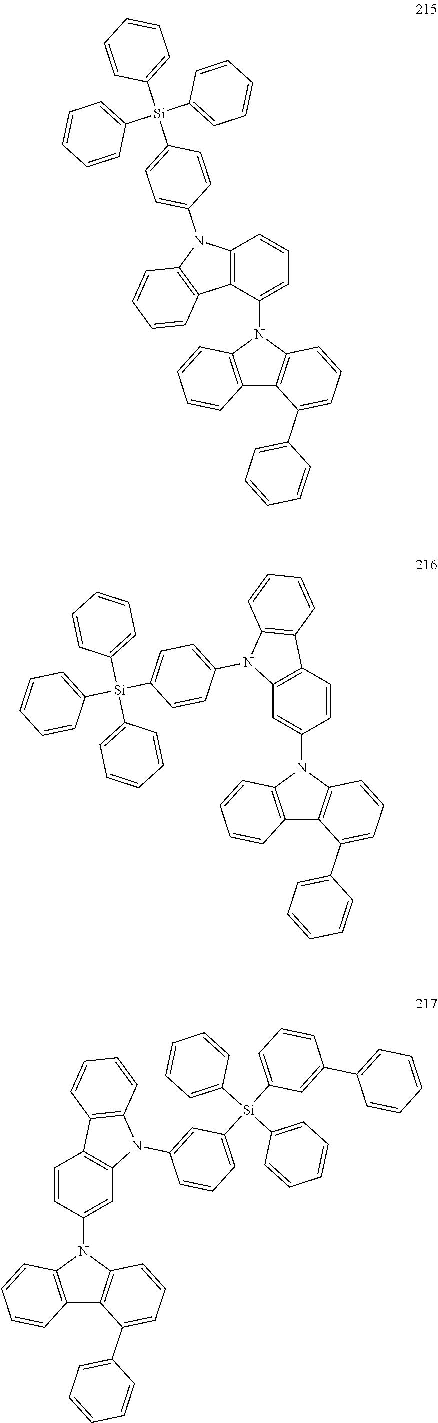 [SMILES: c1ccc(-c2cccc3c2c2ccccc2n3-c2cccc3c2c2ccccc2n3-c2ccc([Si](c3ccccc3)(c3ccccc3)c3ccccc3)cc2)cc1 ; c1ccc(-c2cccc3c2c2ccccc2n3-c2ccc3c4ccccc4n(-c4ccc([Si](c5ccccc5)(c5ccccc5)c5ccccc5)cc4)c3c2)cc1 ; c1ccc(-c2cccc([Si](c3ccccc3)(c3ccccc3)c3cccc(-n4c5ccccc5c5ccc(-n6c7ccccc7c7c(-c8ccccc8)cccc76)cc54)c3)c2)cc1]

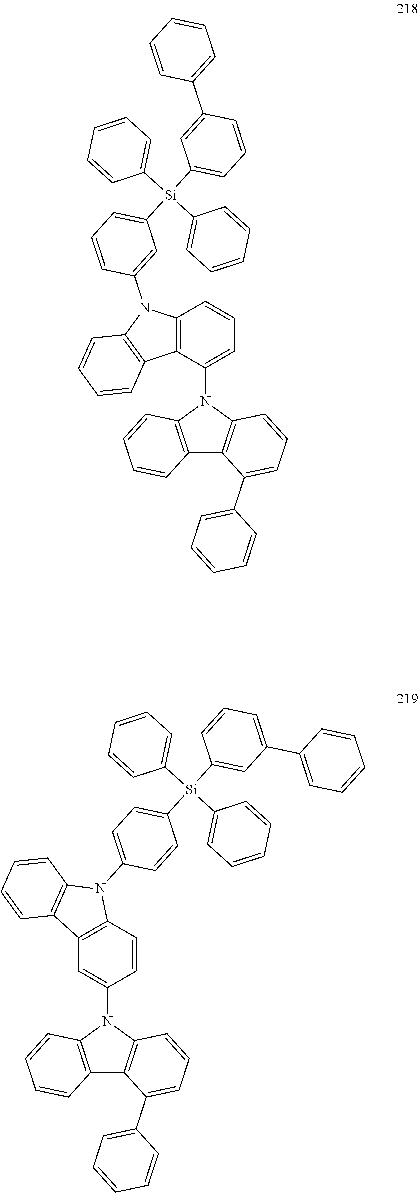 [SMILES: c1ccc(-c2cccc([Si](c3ccccc3)(c3ccccc3)c3cccc(-n4c5ccccc5c5c(-n6c7ccccc7c7c(-c8ccccc8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c(-c8ccccc8)cccc76)ccc54)cc3)c2)cc1]

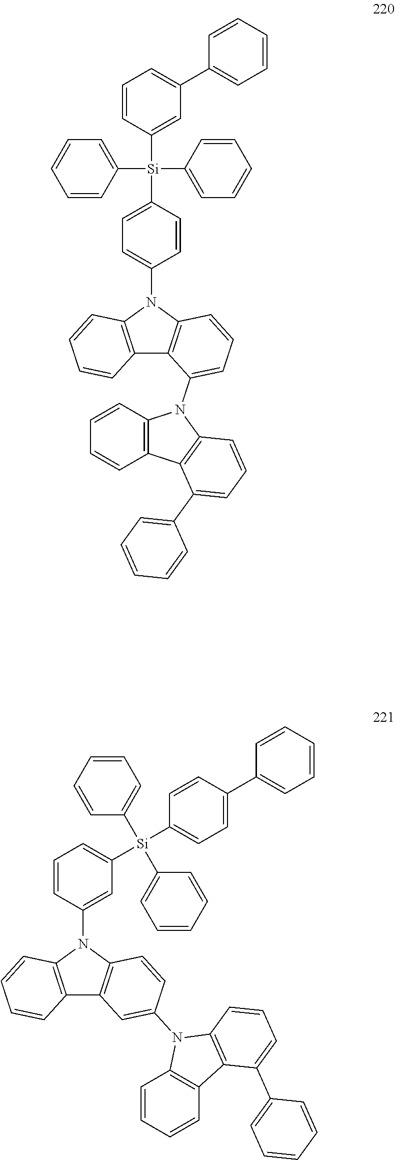 [SMILES: c1ccc(-c2cccc([Si](c3ccccc3)(c3ccccc3)c3ccc(-n4c5ccccc5c5c(-n6c7ccccc7c7c(-c8ccccc8)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c(-c8ccccc8)cccc76)ccc54)c3)cc2)cc1]

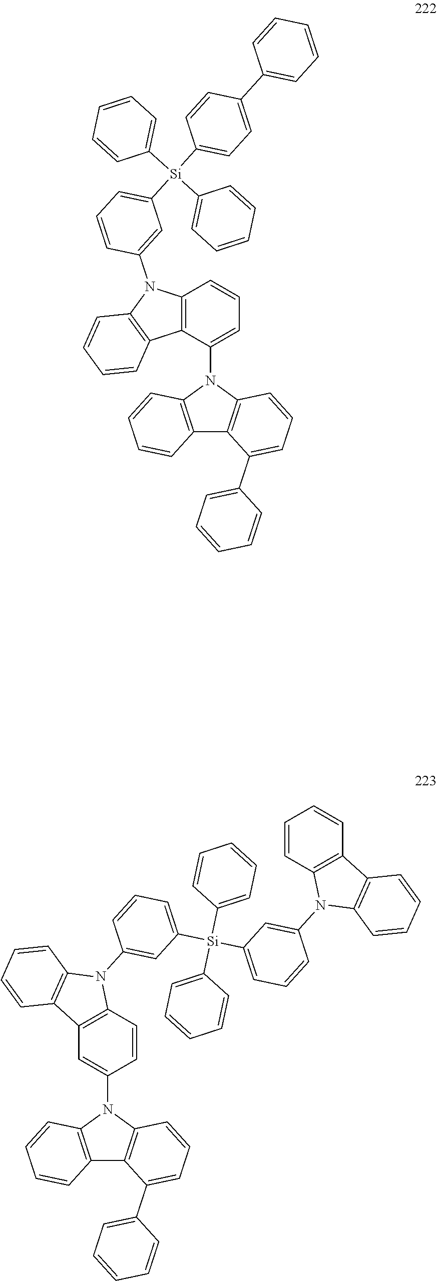 [SMILES: c1ccc(-c2ccc([Si](c3ccccc3)(c3ccccc3)c3cccc(-n4c5ccccc5c5c(-n6c7ccccc7c7c(-c8ccccc8)cccc76)cccc54)c3)cc2)cc1 ; c1ccc(-c2cccc3c2c2ccccc2n3-c2ccc3c(c2)c2ccccc2n3-c2cccc([Si](c3ccccc3)(c3ccccc3)c3cccc(-n4c5ccccc5c5ccccc54)c3)c2)cc1]

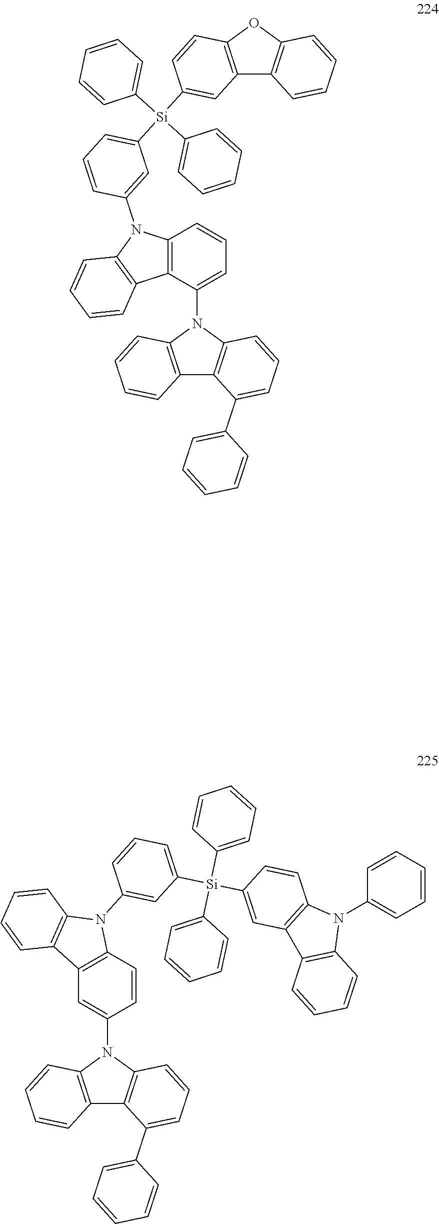 [SMILES: c1ccc(-c2cccc3c2c2ccccc2n3-c2cccc3c2c2ccccc2n3-c2cccc([Si](c3ccccc3)(c3ccccc3)c3ccc4oc5ccccc5c4c3)c2)cc1 ; c1ccc(-c2cccc3c2c2ccccc2n3-c2ccc3c(c2)c2ccccc2n3-c2cccc([Si](c3ccccc3)(c3ccccc3)c3ccc4c(c3)c3ccccc3n4-c3ccccc3)c2)cc1]

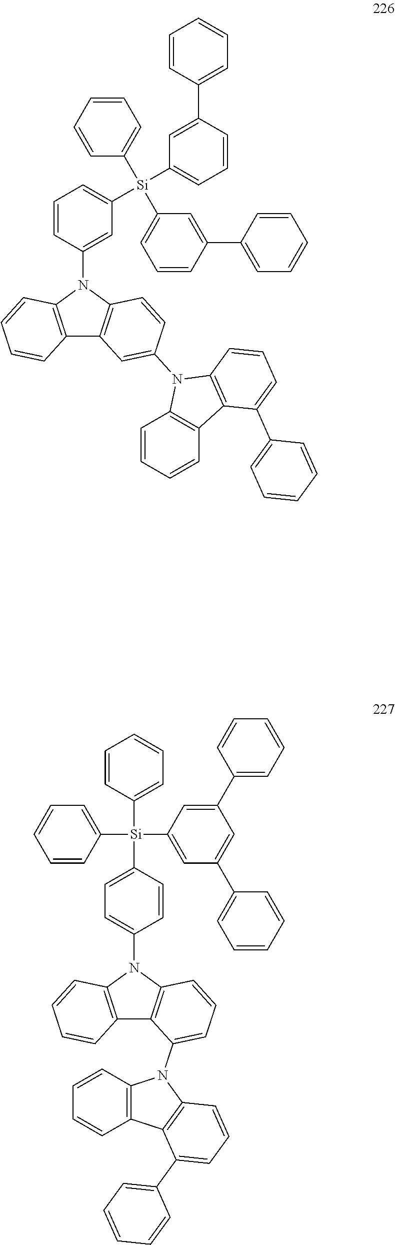 [SMILES: c1ccc(-c2cccc([Si](c3ccccc3)(c3cccc(-c4ccccc4)c3)c3cccc(-n4c5ccccc5c5cc(-n6c7ccccc7c7c(-c8ccccc8)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c(-c8ccccc8)cccc76)cccc54)cc3)c2)cc1]

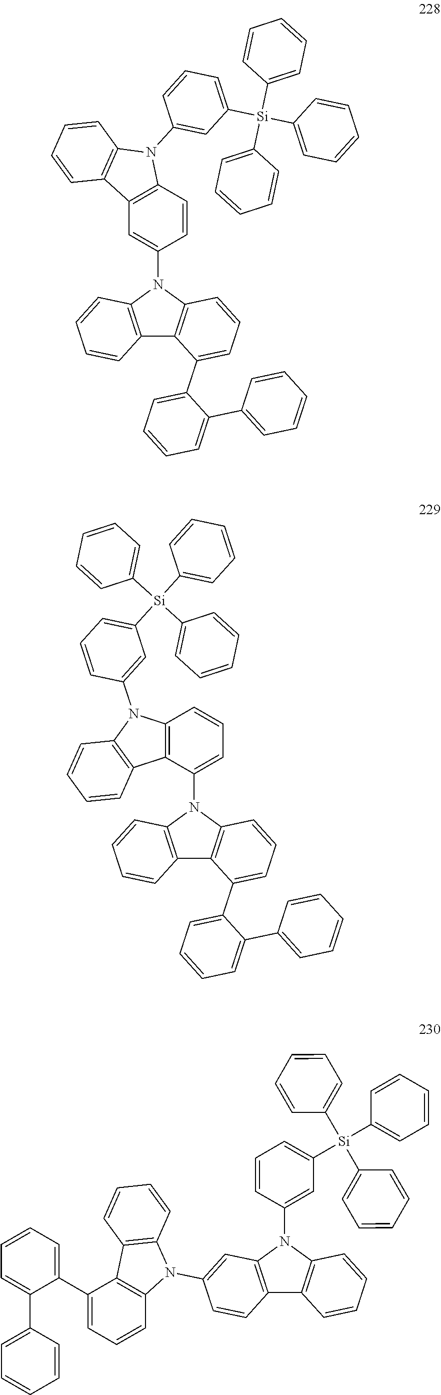 [SMILES: c1ccc(-c2ccccc2-c2cccc3c2c2ccccc2n3-c2ccc3c(c2)c2ccccc2n3-c2cccc([Si](c3ccccc3)(c3ccccc3)c3ccccc3)c2)cc1 ; c1ccc(-c2ccccc2-c2cccc3c2c2ccccc2n3-c2cccc3c2c2ccccc2n3-c2cccc([Si](c3ccccc3)(c3ccccc3)c3ccccc3)c2)cc1 ; c1ccc(-c2ccccc2-c2cccc3c2c2ccccc2n3-c2ccc3c4ccccc4n(-c4cccc([Si](c5ccccc5)(c5ccccc5)c5ccccc5)c4)c3c2)cc1]

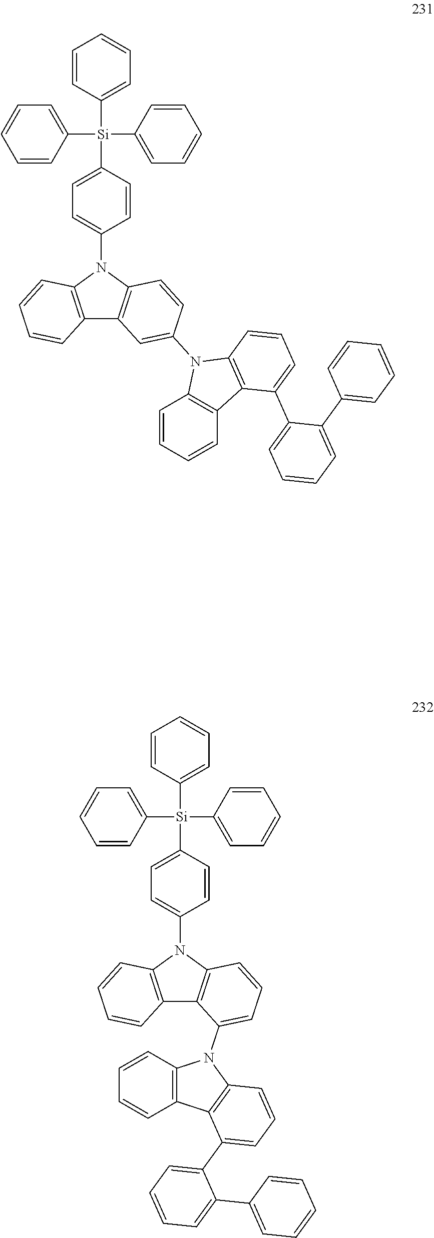 [SMILES: c1ccc(-c2ccccc2-c2cccc3c2c2ccccc2n3-c2ccc3c(c2)c2ccccc2n3-c2ccc([Si](c3ccccc3)(c3ccccc3)c3ccccc3)cc2)cc1 ; c1ccc(-c2ccccc2-c2cccc3c2c2ccccc2n3-c2cccc3c2c2ccccc2n3-c2ccc([Si](c3ccccc3)(c3ccccc3)c3ccccc3)cc2)cc1]

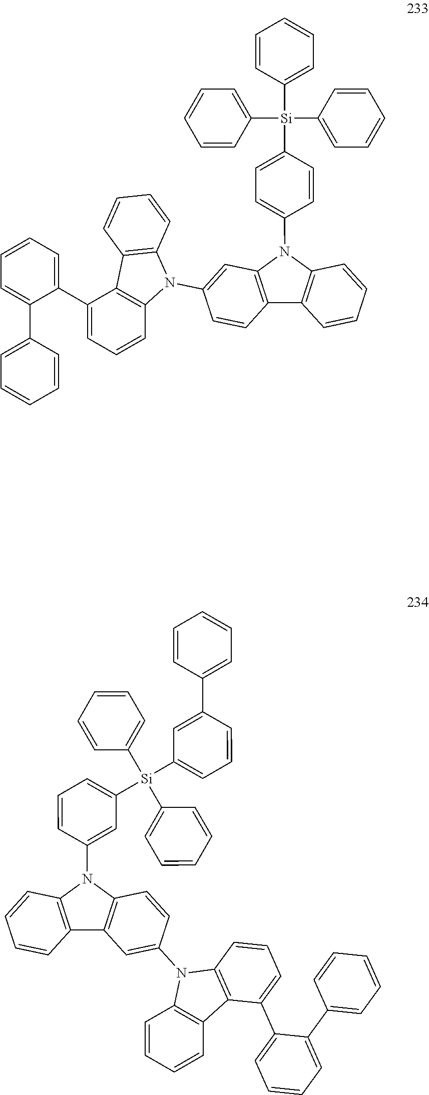 [SMILES: c1ccc(-c2ccccc2-c2cccc3c2c2ccccc2n3-c2ccc3c4ccccc4n(-c4ccc([Si](c5ccccc5)(c5ccccc5)c5ccccc5)cc4)c3c2)cc1 ; c1ccc(-c2cccc([Si](c3ccccc3)(c3ccccc3)c3cccc(-n4c5ccccc5c5cc(-n6c7ccccc7c7c(-c8ccccc8-c8ccccc8)cccc76)ccc54)c3)c2)cc1]

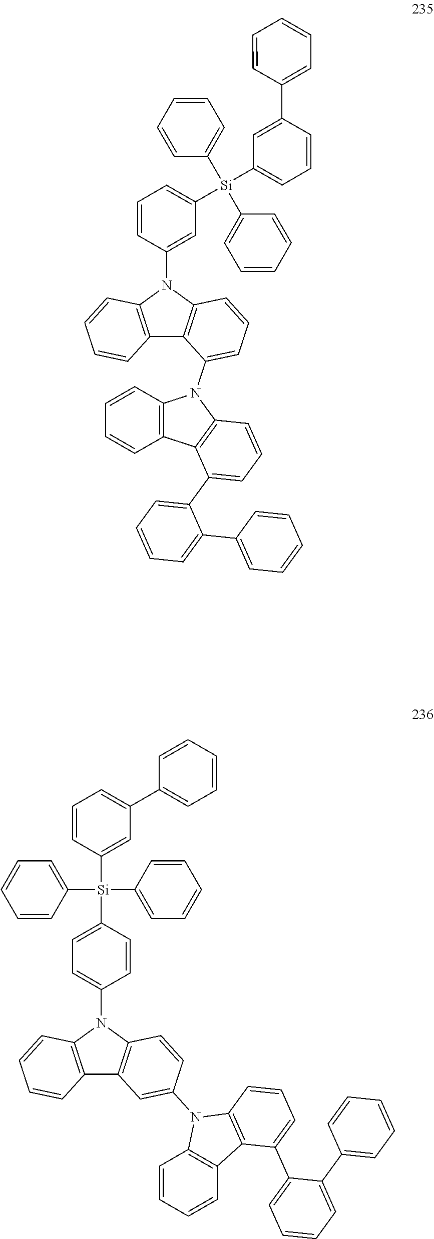 [SMILES: c1ccc(-c2cccc([Si](c3ccccc3)(c3ccccc3)c3cccc(-n4c5ccccc5c5c(-n6c7ccccc7c7c(-c8ccccc8-c8ccccc8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c(-c8ccccc8-c8ccccc8)cccc76)ccc54)cc3)c2)cc1]

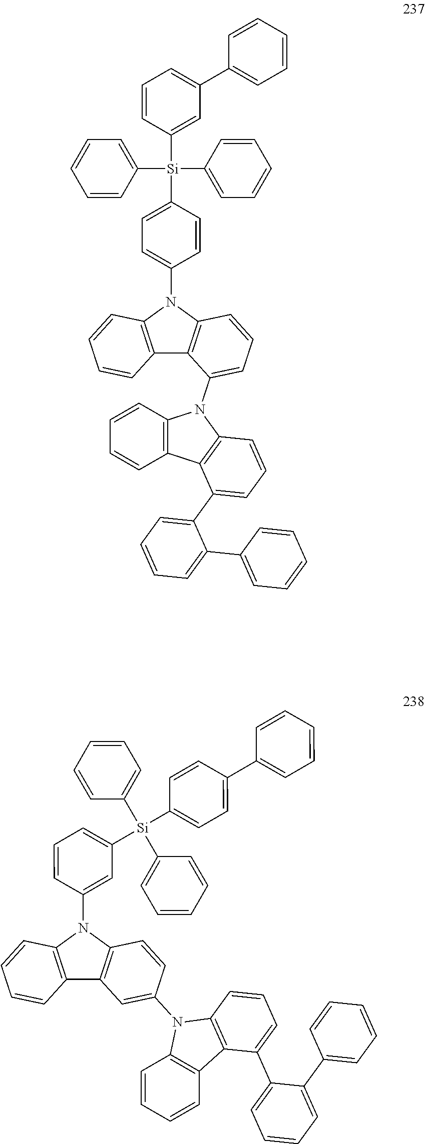 [SMILES: c1ccc(-c2cccc([Si](c3ccccc3)(c3ccccc3)c3ccc(-n4c5ccccc5c5c(-n6c7ccccc7c7c(-c8ccccc8-c8ccccc8)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c(-c8ccccc8-c8ccccc8)cccc76)ccc54)c3)cc2)cc1]

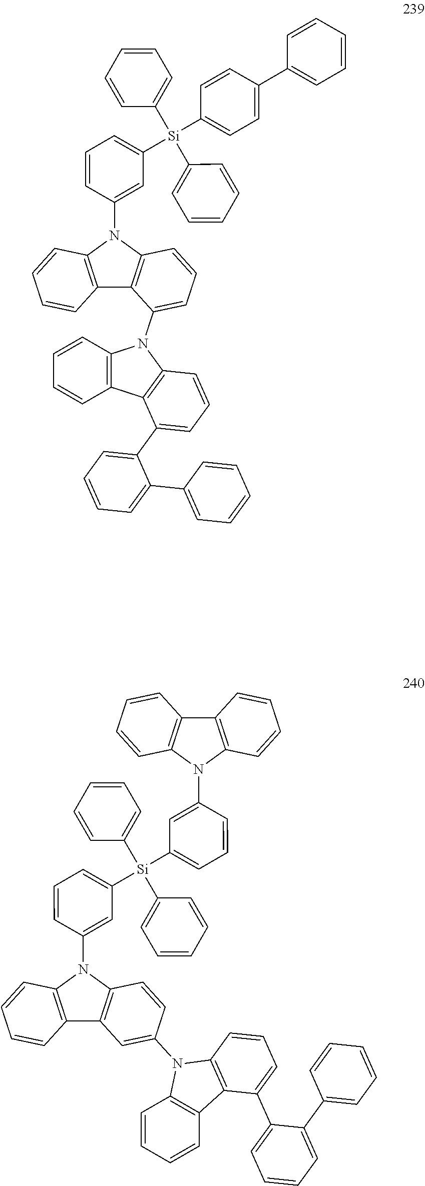 [SMILES: c1ccc(-c2ccc([Si](c3ccccc3)(c3ccccc3)c3cccc(-n4c5ccccc5c5c(-n6c7ccccc7c7c(-c8ccccc8-c8ccccc8)cccc76)cccc54)c3)cc2)cc1 ; c1ccc(-c2ccccc2-c2cccc3c2c2ccccc2n3-c2ccc3c(c2)c2ccccc2n3-c2cccc([Si](c3ccccc3)(c3ccccc3)c3cccc(-n4c5ccccc5c5ccccc54)c3)c2)cc1]

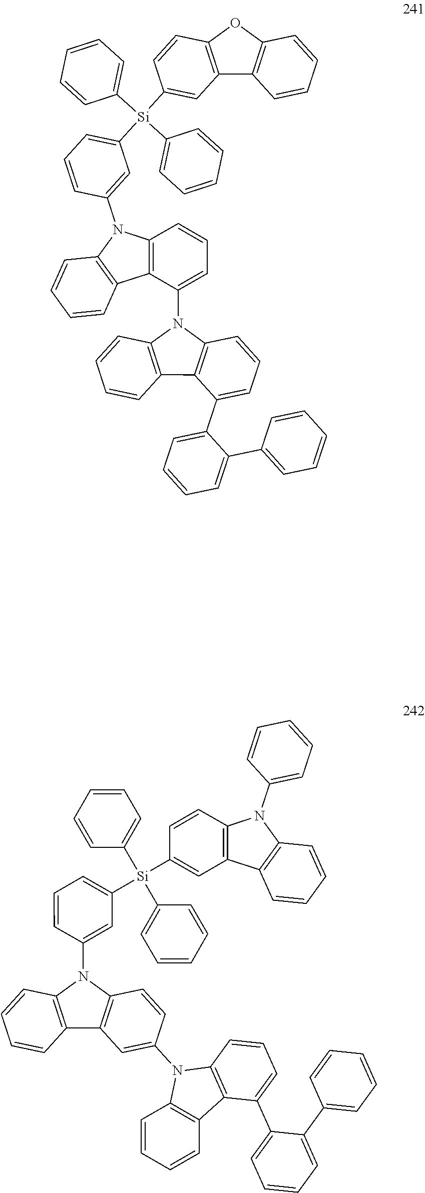 [SMILES: c1ccc(-c2ccccc2-c2cccc3c2c2ccccc2n3-c2cccc3c2c2ccccc2n3-c2cccc([Si](c3ccccc3)(c3ccccc3)c3ccc4oc5ccccc5c4c3)c2)cc1 ; c1ccc(-c2ccccc2-c2cccc3c2c2ccccc2n3-c2ccc3c(c2)c2ccccc2n3-c2cccc([Si](c3ccccc3)(c3ccccc3)c3ccc4c(c3)c3ccccc3n4-c3ccccc3)c2)cc1]

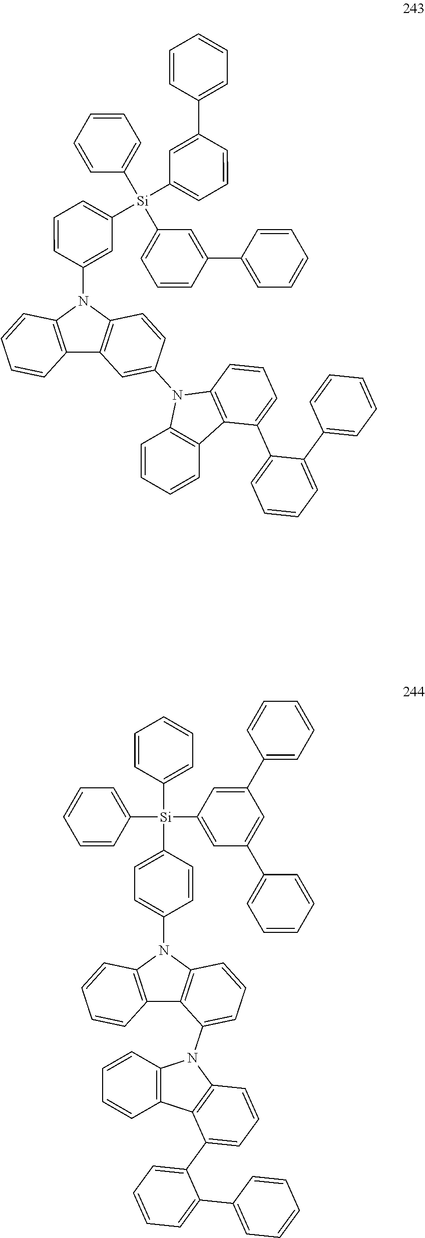 [SMILES: c1ccc(-c2cccc([Si](c3ccccc3)(c3cccc(-c4ccccc4)c3)c3cccc(-n4c5ccccc5c5cc(-n6c7ccccc7c7c(-c8ccccc8-c8ccccc8)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c(-c8ccccc8-c8ccccc8)cccc76)cccc54)cc3)c2)cc1]

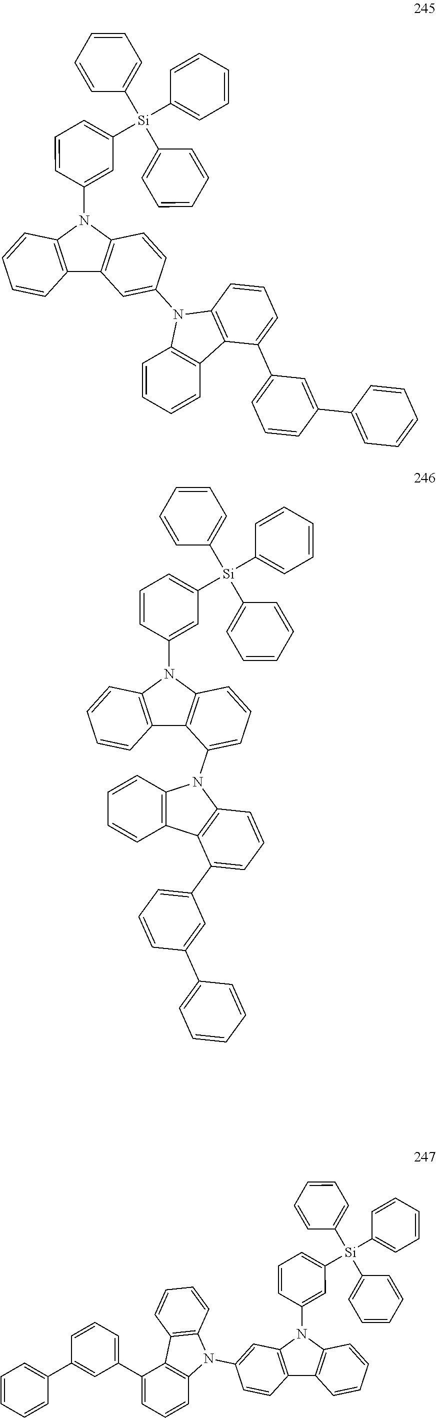 [SMILES: c1ccc(-c2cccc(-c3cccc4c3c3ccccc3n4-c3ccc4c(c3)c3ccccc3n4-c3cccc([Si](c4ccccc4)(c4ccccc4)c4ccccc4)c3)c2)cc1 ; c1ccc(-c2cccc(-c3cccc4c3c3ccccc3n4-c3cccc4c3c3ccccc3n4-c3cccc([Si](c4ccccc4)(c4ccccc4)c4ccccc4)c3)c2)cc1 ; c1ccc(-c2cccc(-c3cccc4c3c3ccccc3n4-c3ccc4c5ccccc5n(-c5cccc([Si](c6ccccc6)(c6ccccc6)c6ccccc6)c5)c4c3)c2)cc1]

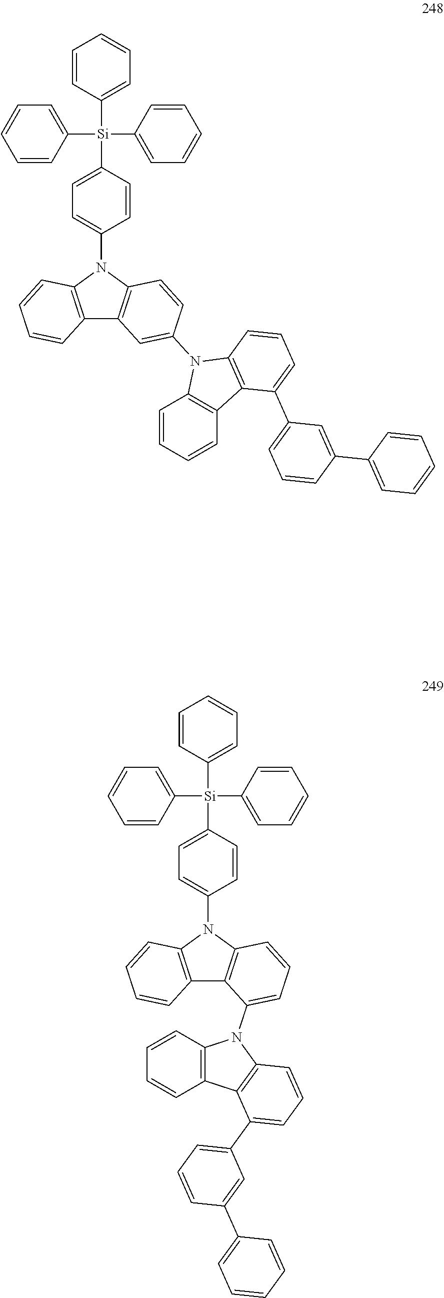 [SMILES: c1ccc(-c2cccc(-c3cccc4c3c3ccccc3n4-c3ccc4c(c3)c3ccccc3n4-c3ccc([Si](c4ccccc4)(c4ccccc4)c4ccccc4)cc3)c2)cc1 ; c1ccc(-c2cccc(-c3cccc4c3c3ccccc3n4-c3cccc4c3c3ccccc3n4-c3ccc([Si](c4ccccc4)(c4ccccc4)c4ccccc4)cc3)c2)cc1]

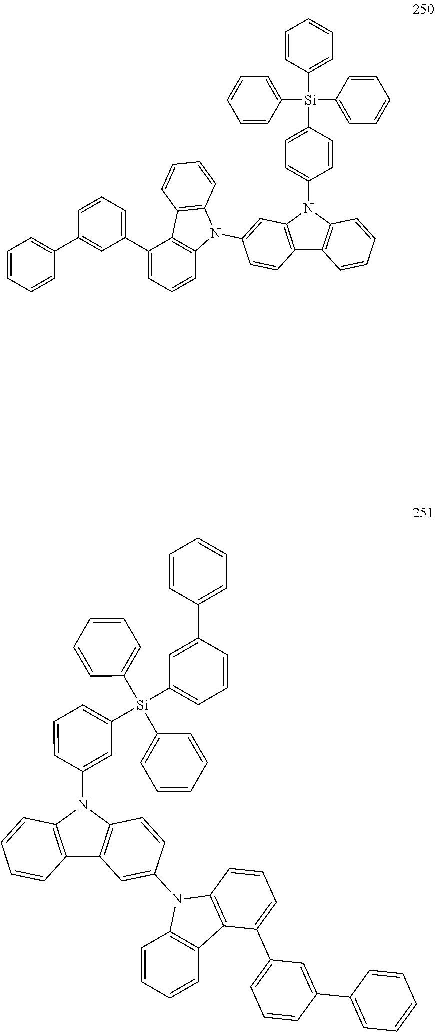 [SMILES: c1ccc(-c2cccc(-c3cccc4c3c3ccccc3n4-c3ccc4c5ccccc5n(-c5ccc([Si](c6ccccc6)(c6ccccc6)c6ccccc6)cc5)c4c3)c2)cc1 ; c1ccc(-c2cccc(-c3cccc4c3c3ccccc3n4-c3ccc4c(c3)c3ccccc3n4-c3cccc([Si](c4ccccc4)(c4ccccc4)c4cccc(-c5ccccc5)c4)c3)c2)cc1]

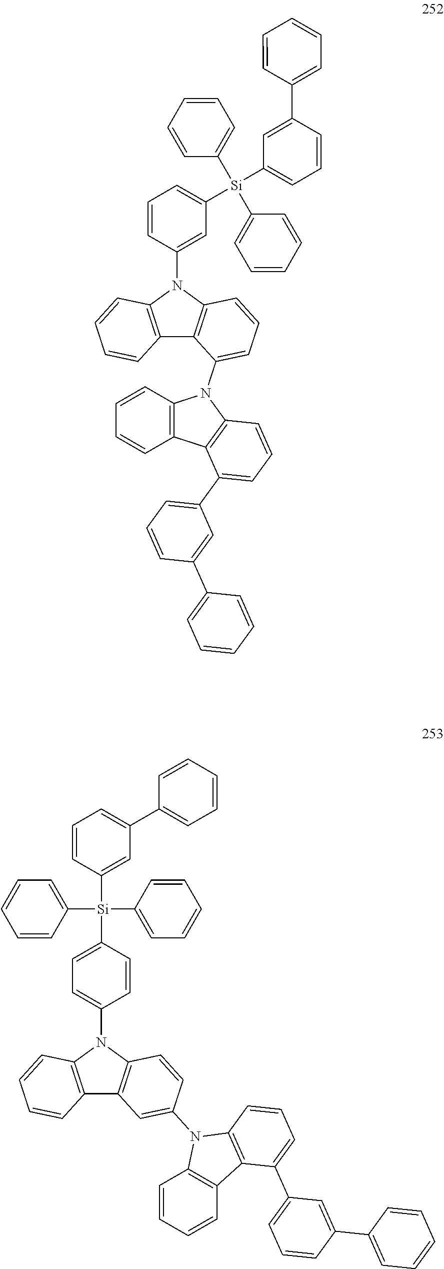 [SMILES: c1ccc(-c2cccc(-c3cccc4c3c3ccccc3n4-c3cccc4c3c3ccccc3n4-c3cccc([Si](c4ccccc4)(c4ccccc4)c4cccc(-c5ccccc5)c4)c3)c2)cc1 ; c1ccc(-c2cccc(-c3cccc4c3c3ccccc3n4-c3ccc4c(c3)c3ccccc3n4-c3ccc([Si](c4ccccc4)(c4ccccc4)c4cccc(-c5ccccc5)c4)cc3)c2)cc1]

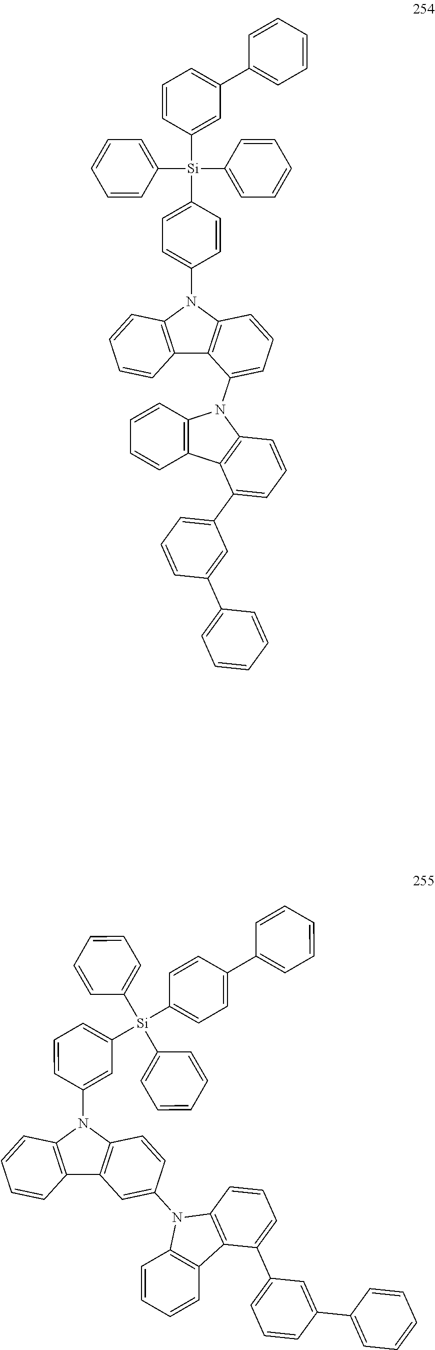 [SMILES: c1ccc(-c2cccc(-c3cccc4c3c3ccccc3n4-c3cccc4c3c3ccccc3n4-c3ccc([Si](c4ccccc4)(c4ccccc4)c4cccc(-c5ccccc5)c4)cc3)c2)cc1 ; c1ccc(-c2ccc([Si](c3ccccc3)(c3ccccc3)c3cccc(-n4c5ccccc5c5cc(-n6c7ccccc7c7c(-c8cccc(-c9ccccc9)c8)cccc76)ccc54)c3)cc2)cc1]

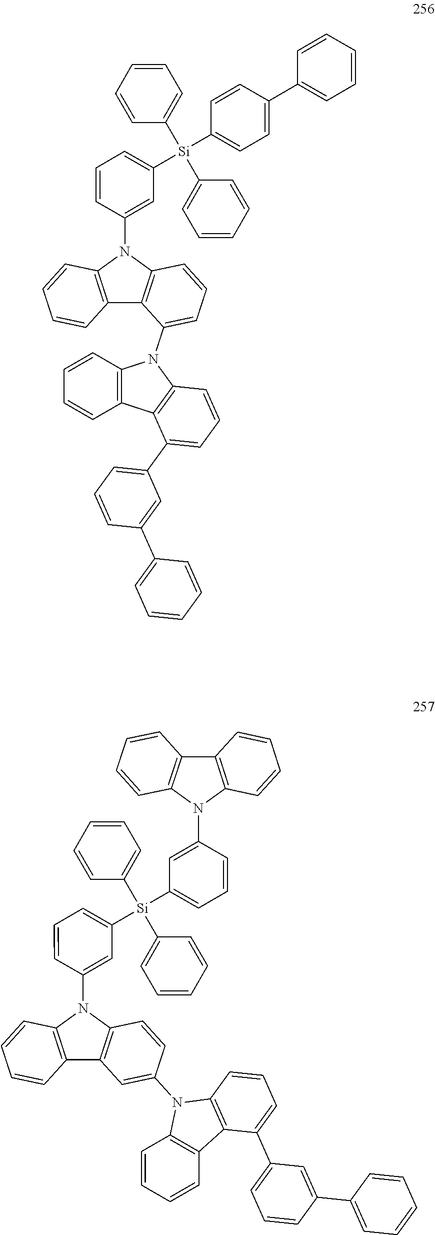 [SMILES: c1ccc(-c2ccc([Si](c3ccccc3)(c3ccccc3)c3cccc(-n4c5ccccc5c5c(-n6c7ccccc7c7c(-c8cccc(-c9ccccc9)c8)cccc76)cccc54)c3)cc2)cc1 ; c1ccc(-c2cccc(-c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)c2)cc1]

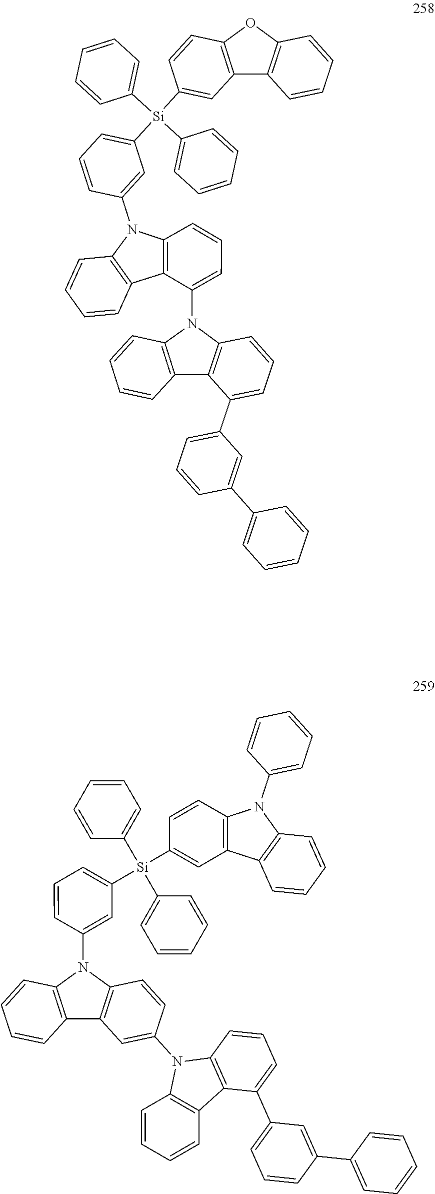 [SMILES: c1ccc(-c2cccc(-c3cccc4c3c3ccccc3n4-c3cccc4c3c3ccccc3n4-c3cccc([Si](c4ccccc4)(c4ccccc4)c4ccc5oc6ccccc6c5c4)c3)c2)cc1 ; c1ccc(-c2cccc(-c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)c2)cc1]

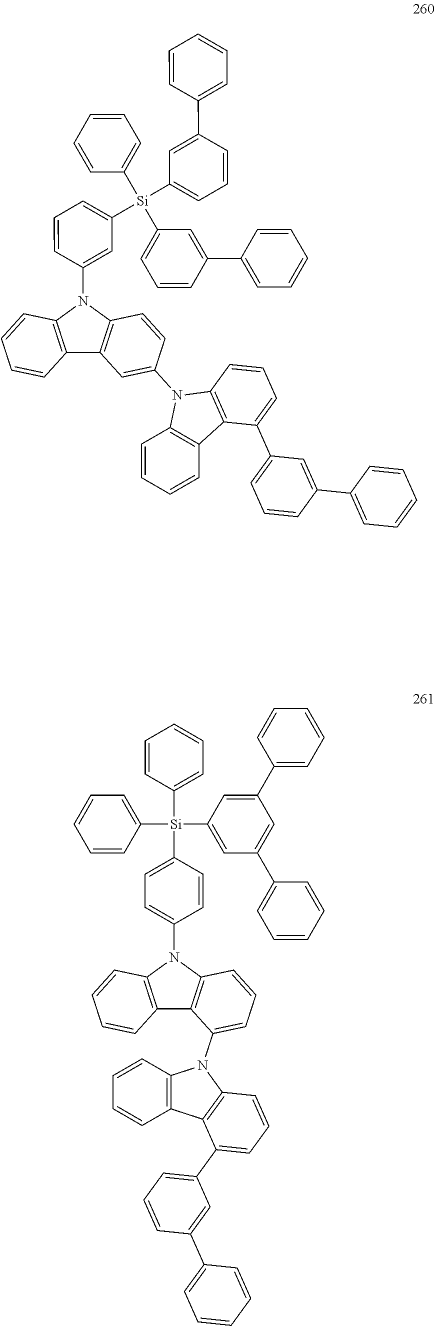 [SMILES: c1ccc(-c2cccc(-c3cccc4c3c3ccccc3n4-c3ccc4c(c3)c3ccccc3n4-c3cccc([Si](c4ccccc4)(c4cccc(-c5ccccc5)c4)c4cccc(-c5ccccc5)c4)c3)c2)cc1 ; c1ccc(-c2cccc(-c3cccc4c3c3ccccc3n4-c3cccc4c3c3ccccc3n4-c3ccc([Si](c4ccccc4)(c4ccccc4)c4cc(-c5ccccc5)cc(-c5ccccc5)c4)cc3)c2)cc1]

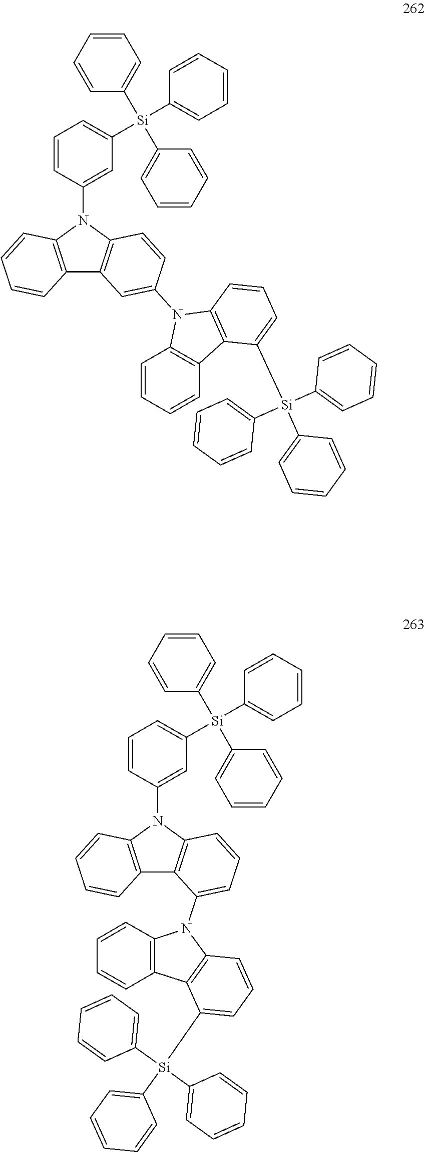 [SMILES: c1ccc([Si](c2ccccc2)(c2ccccc2)c2cccc(-n3c4ccccc4c4cc(-n5c6ccccc6c6c([Si](c7ccccc7)(c7ccccc7)c7ccccc7)cccc65)ccc43)c2)cc1 ; c1ccc([Si](c2ccccc2)(c2ccccc2)c2cccc(-n3c4ccccc4c4c(-n5c6ccccc6c6c([Si](c7ccccc7)(c7ccccc7)c7ccccc7)cccc65)cccc43)c2)cc1]

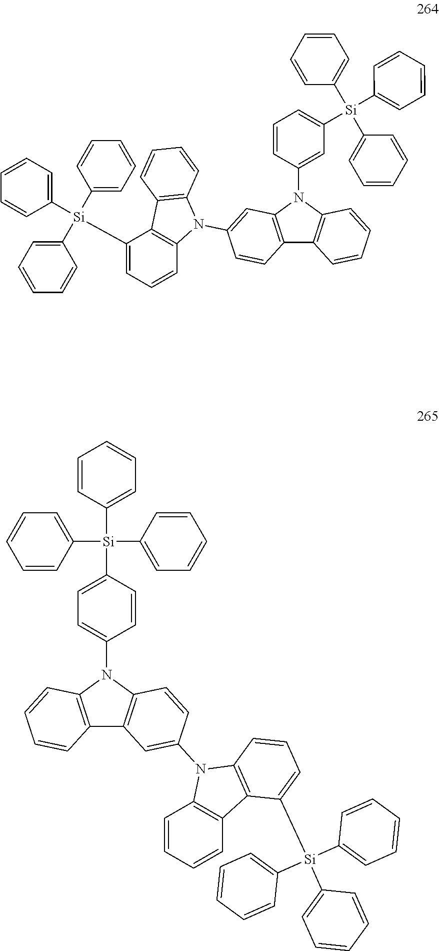 [SMILES: c1ccc([Si](c2ccccc2)(c2ccccc2)c2cccc(-n3c4ccccc4c4ccc(-n5c6ccccc6c6c([Si](c7ccccc7)(c7ccccc7)c7ccccc7)cccc65)cc43)c2)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7ccccc7)cccc65)ccc43)cc2)cc1]

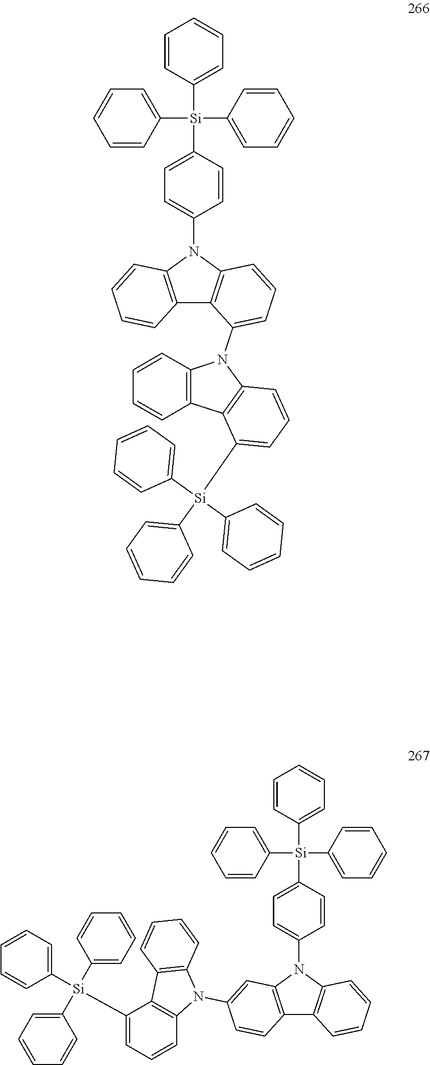 [SMILES: c1ccc([Si](c2ccccc2)(c2ccccc2)c2ccc(-n3c4ccccc4c4c(-n5c6ccccc6c6c([Si](c7ccccc7)(c7ccccc7)c7ccccc7)cccc65)cccc43)cc2)cc1 ; c1ccc([Si](c2ccccc2)(c2ccccc2)c2ccc(-n3c4ccccc4c4ccc(-n5c6ccccc6c6c([Si](c7ccccc7)(c7ccccc7)c7ccccc7)cccc65)cc43)cc2)cc1]

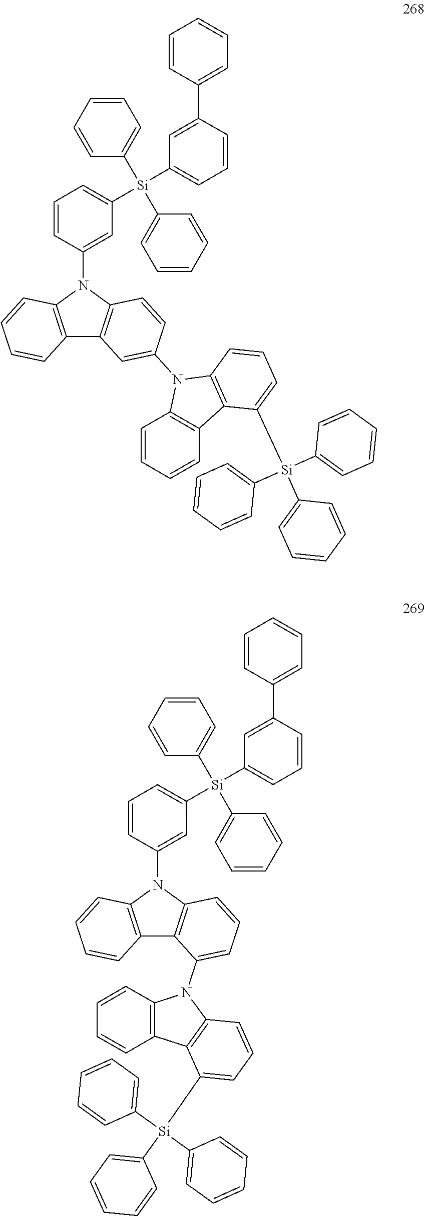 [SMILES: c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8ccccc8)cccc76)ccc54)c3)c2)cc1 ; c1ccc(-c2cccc([Si](c3ccccc3)(c3ccccc3)c3cccc(-n4c5ccccc5c5c(-n6c7ccccc7c7c([Si](c8ccccc8)(c8ccccc8)c8ccccc8)cccc76)cccc54)c3)c2)cc1]

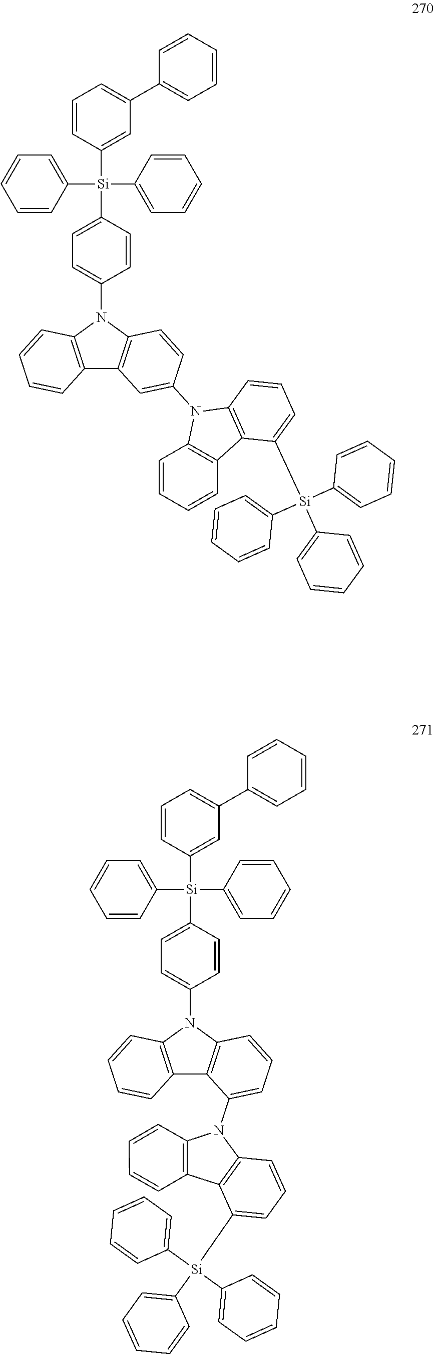 [SMILES: c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8ccccc8)cccc76)ccc54)cc3)c2)cc1 ; c1ccc(-c2cccc([Si](c3ccccc3)(c3ccccc3)c3ccc(-n4c5ccccc5c5c(-n6c7ccccc7c7c([Si](c8ccccc8)(c8ccccc8)c8ccccc8)cccc76)cccc54)cc3)c2)cc1]

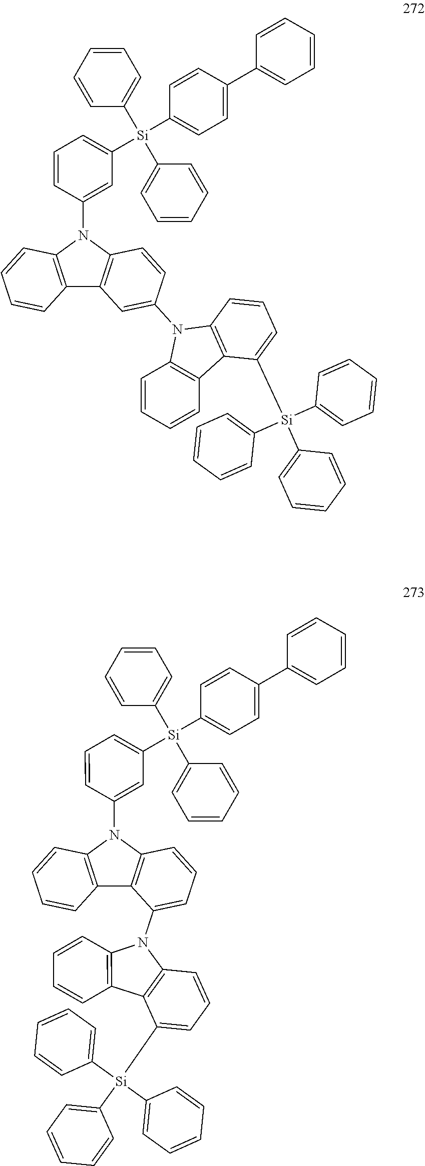 [SMILES: c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccccc8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8ccccc8)cccc76)cccc54)c3)cc2)cc1]

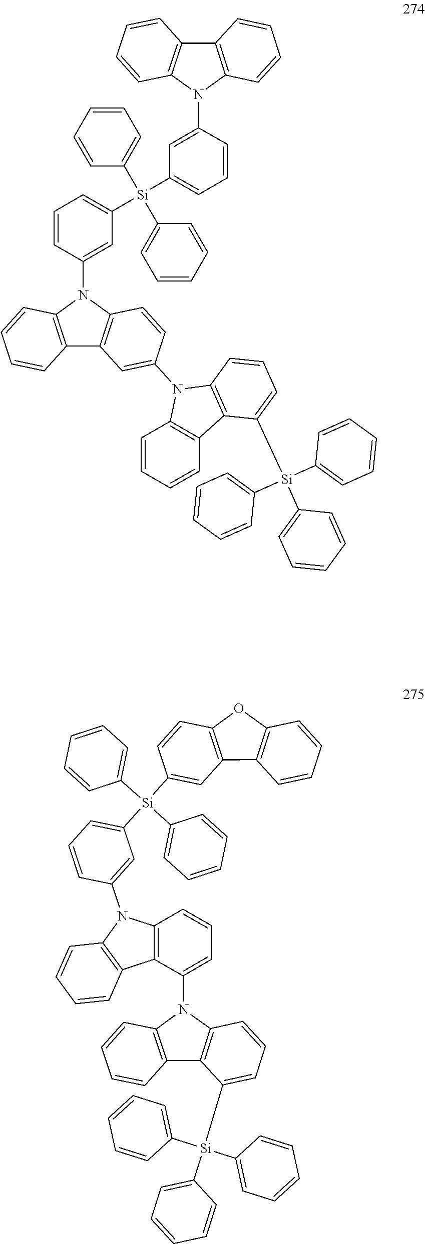 [SMILES: c1ccc([Si](c2ccccc2)(c2cccc(-n3c4ccccc4c4ccccc43)c2)c2cccc(-n3c4ccccc4c4cc(-n5c6ccccc6c6c([Si](c7ccccc7)(c7ccccc7)c7ccccc7)cccc65)ccc43)c2)cc1 ; c1ccc([Si](c2ccccc2)(c2cccc(-n3c4ccccc4c4c(-n5c6ccccc6c6c([Si](c7ccccc7)(c7ccccc7)c7ccccc7)cccc65)cccc43)c2)c2ccc3oc4ccccc4c3c2)cc1]

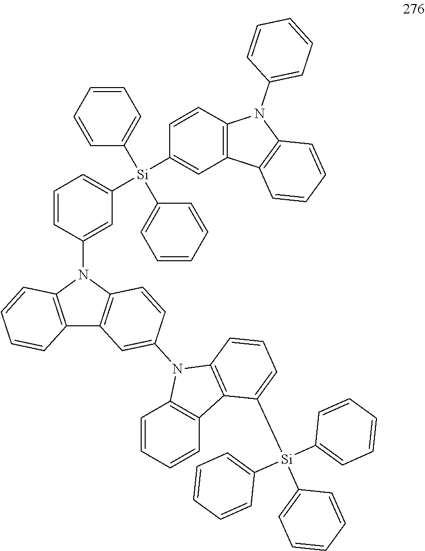 [SMILES: c1ccc(-n2c3ccccc3c3cc([Si](c4ccccc4)(c4ccccc4)c4cccc(-n5c6ccccc6c6cc(-n7c8ccccc8c8c([Si](c9ccccc9)(c9ccccc9)c9ccccc9)cccc87)ccc65)c4)ccc32)cc1]

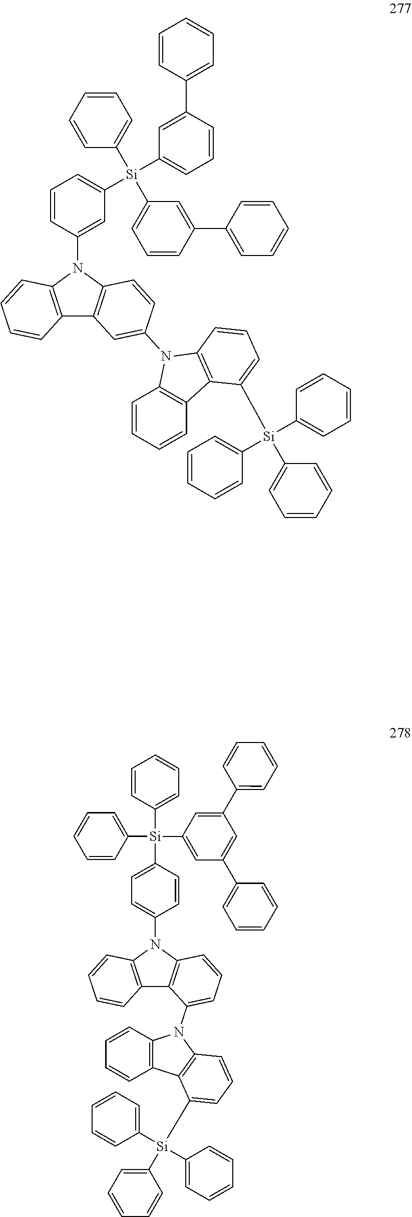 [SMILES: c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8ccccc8)cccc76)ccc54)c3)c2)cc1 ; c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8ccccc8)cccc76)cccc54)cc3)c2)cc1]

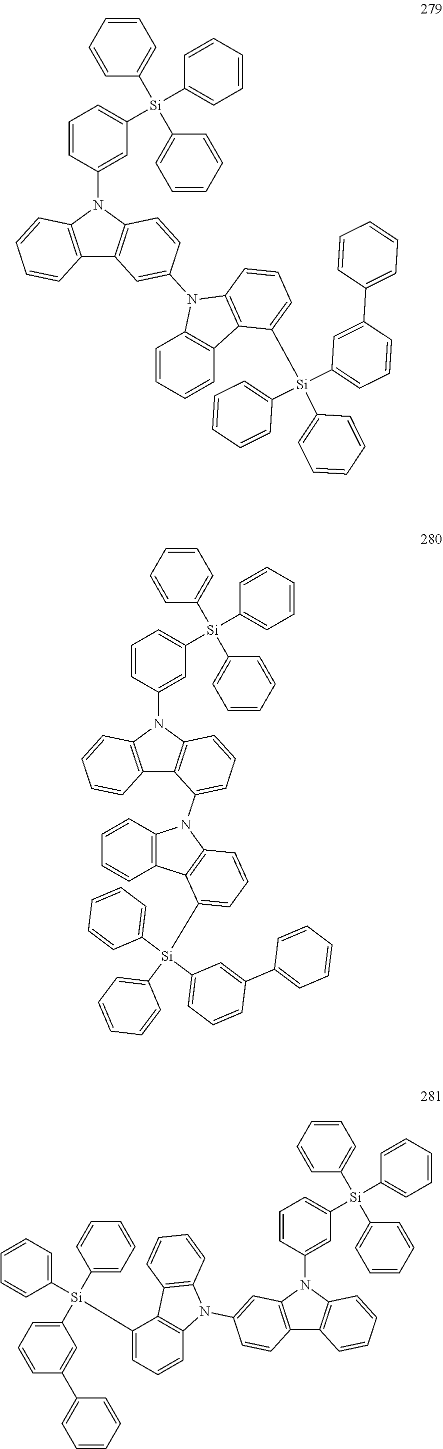

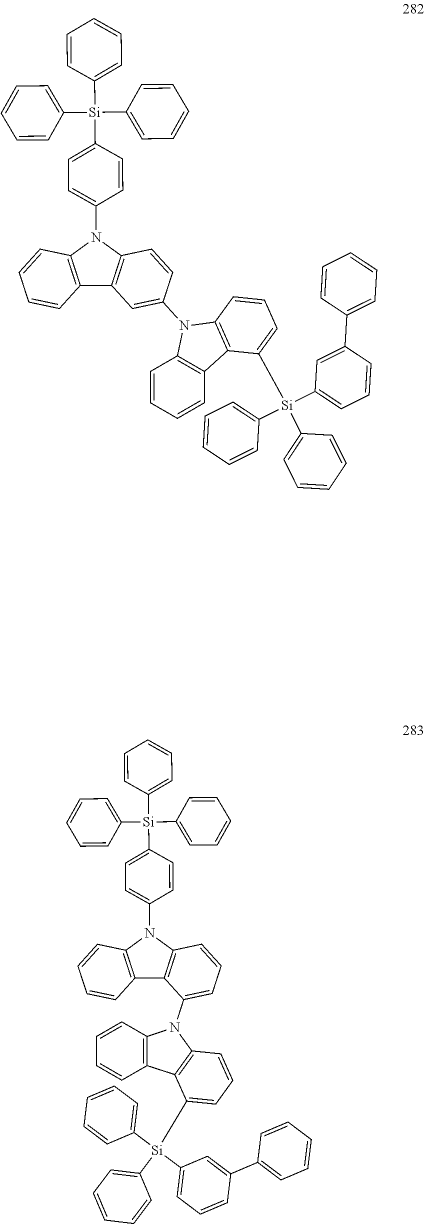 [SMILES: c1ccc(-c2cccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4ccccc4)cc3)c2)cc1 ; c1ccc(-c2cccc([Si](c3ccccc3)(c3ccccc3)c3cccc4c3c3ccccc3n4-c3cccc4c3c3ccccc3n4-c3ccc([Si](c4ccccc4)(c4ccccc4)c4ccccc4)cc3)c2)cc1]

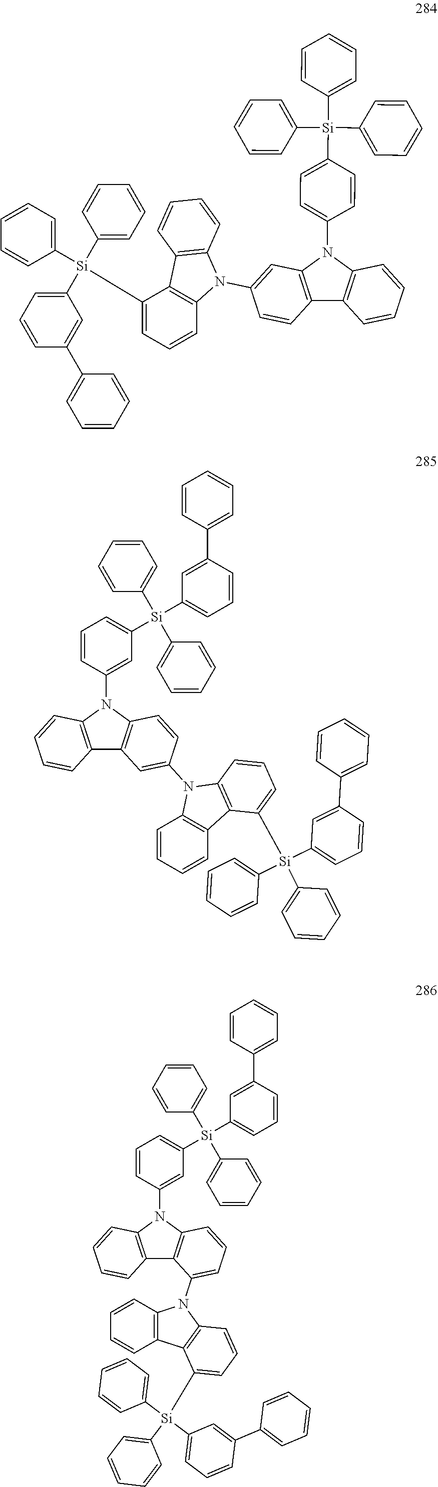 [SMILES: c1ccc(-c2cccc([Si](c3ccccc3)(c3ccccc3)c3cccc4c3c3ccccc3n4-c3ccc4c5ccccc5n(-c5ccc([Si](c6ccccc6)(c6ccccc6)c6ccccc6)cc5)c4c3)c2)cc1 ; c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1 ; c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc(-c9ccccc9)c8)cccc76)cccc54)c3)c2)cc1]

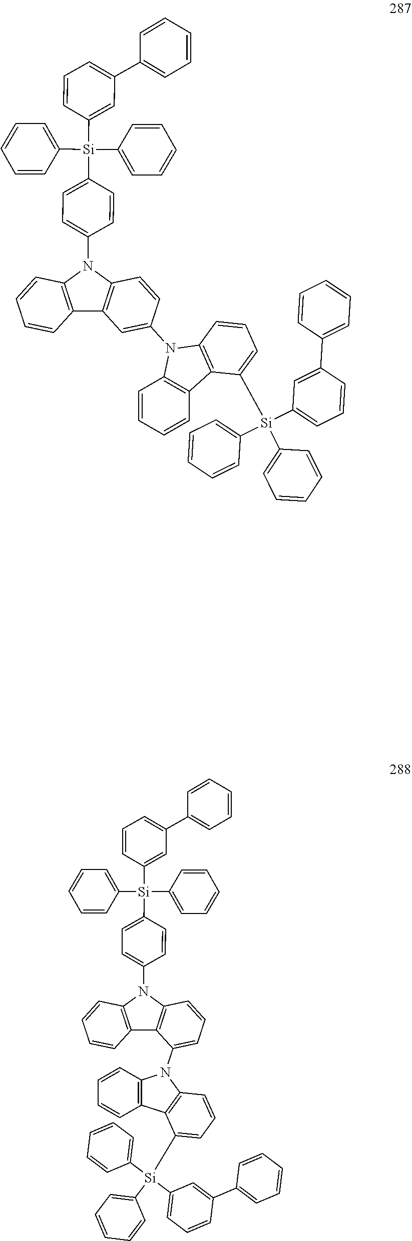 [SMILES: c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8cccc(-c9ccccc9)c8)cccc76)ccc54)cc3)c2)cc1 ; c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc(-c9ccccc9)c8)cccc76)cccc54)cc3)c2)cc1]

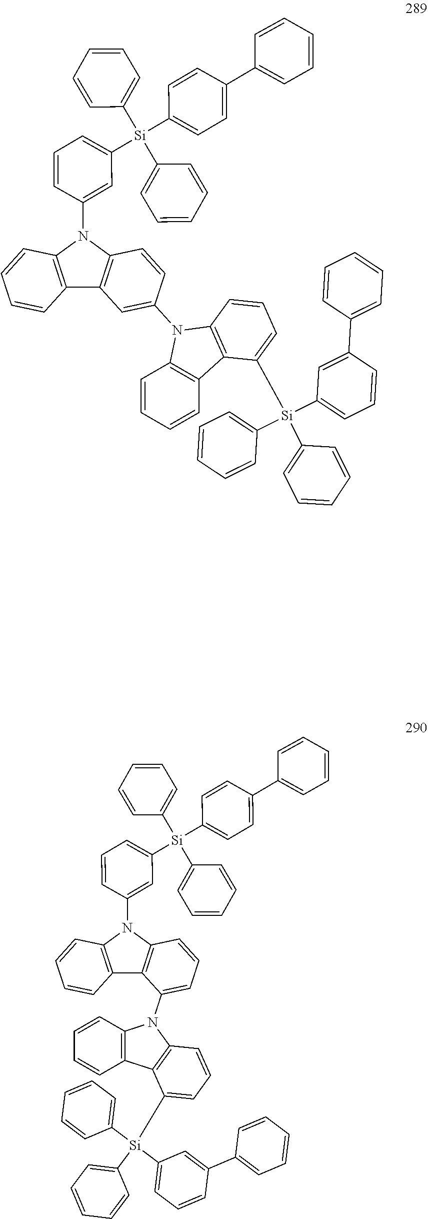 [SMILES: c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8cccc(-c9ccccc9)c8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc(-c9ccccc9)c8)cccc76)cccc54)c3)cc2)cc1]

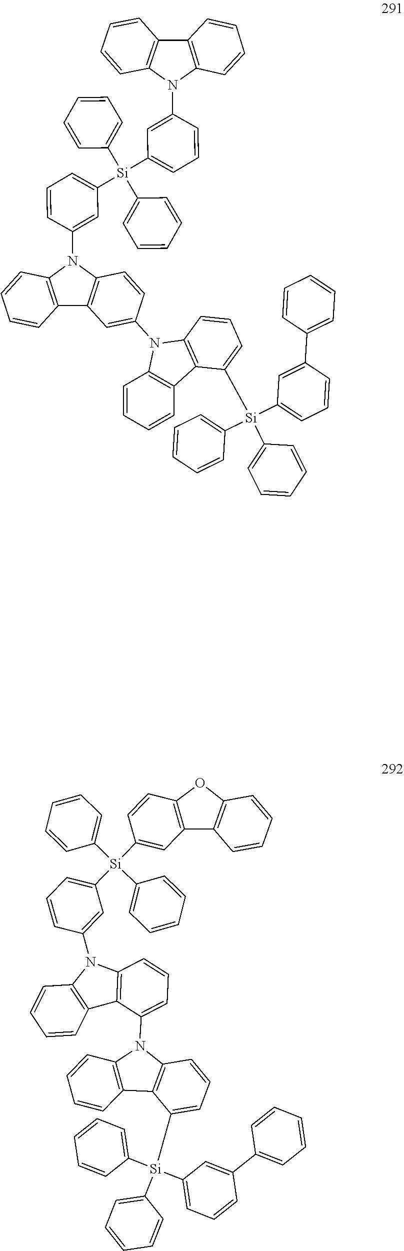 [SMILES: c1ccc(-c2cccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)c2)cc1 ; c1ccc(-c2cccc([Si](c3ccccc3)(c3ccccc3)c3cccc4c3c3ccccc3n4-c3cccc4c3c3ccccc3n4-c3cccc([Si](c4ccccc4)(c4ccccc4)c4ccc5oc6ccccc6c5c4)c3)c2)cc1]

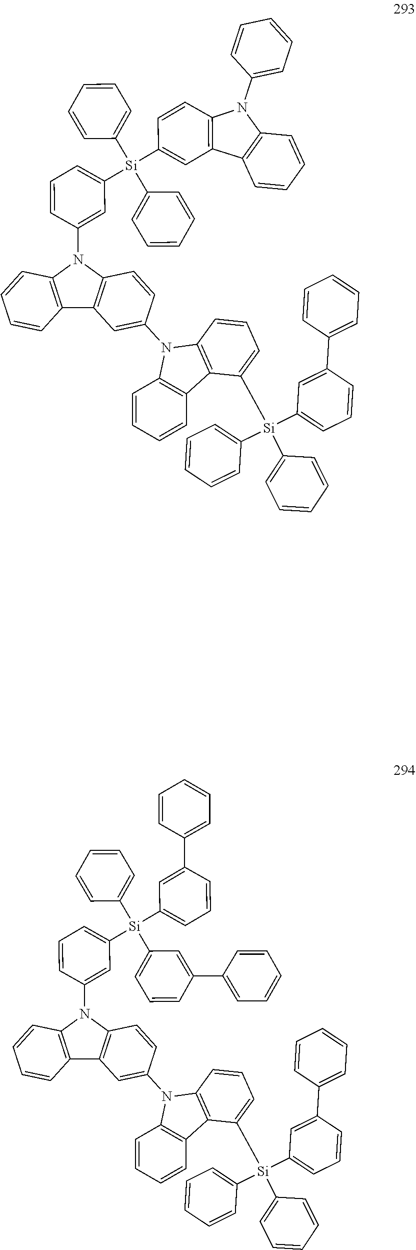 [SMILES: c1ccc(-c2cccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)c2)cc1 ; c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1]

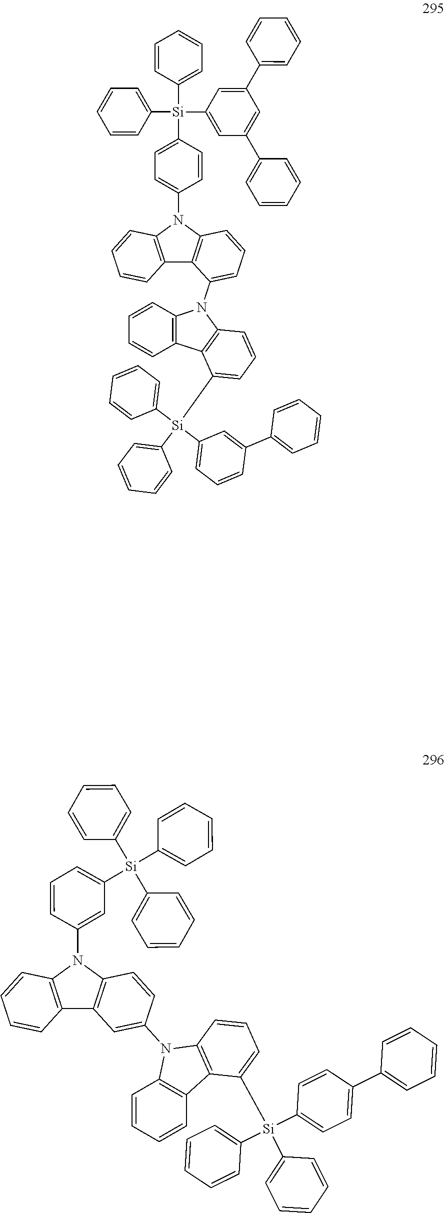 [SMILES: c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8cccc(-c9ccccc9)c8)cccc76)cccc54)cc3)c2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccccc4)c3)cc2)cc1]

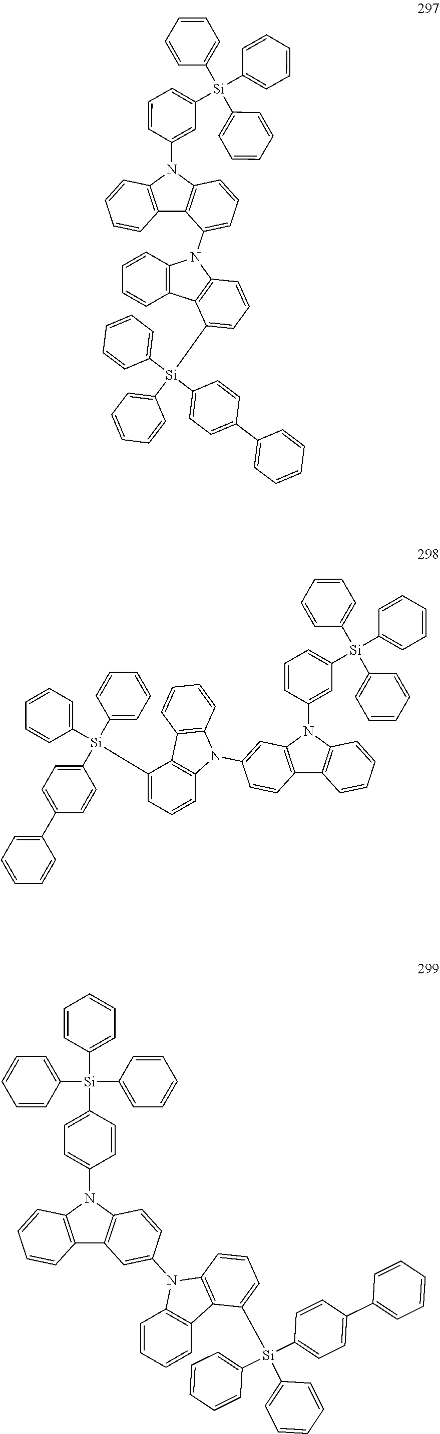 [SMILES: c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4ccccc4)c3)cc2)cc1 ; c1ccc(-c2ccc([Si](c3ccccc3)(c3ccccc3)c3cccc4c3c3ccccc3n4-c3ccc4c5ccccc5n(-c5cccc([Si](c6ccccc6)(c6ccccc6)c6ccccc6)c5)c4c3)cc2)cc1 ; c1ccc(-c2ccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4ccccc4)cc3)cc2)cc1]

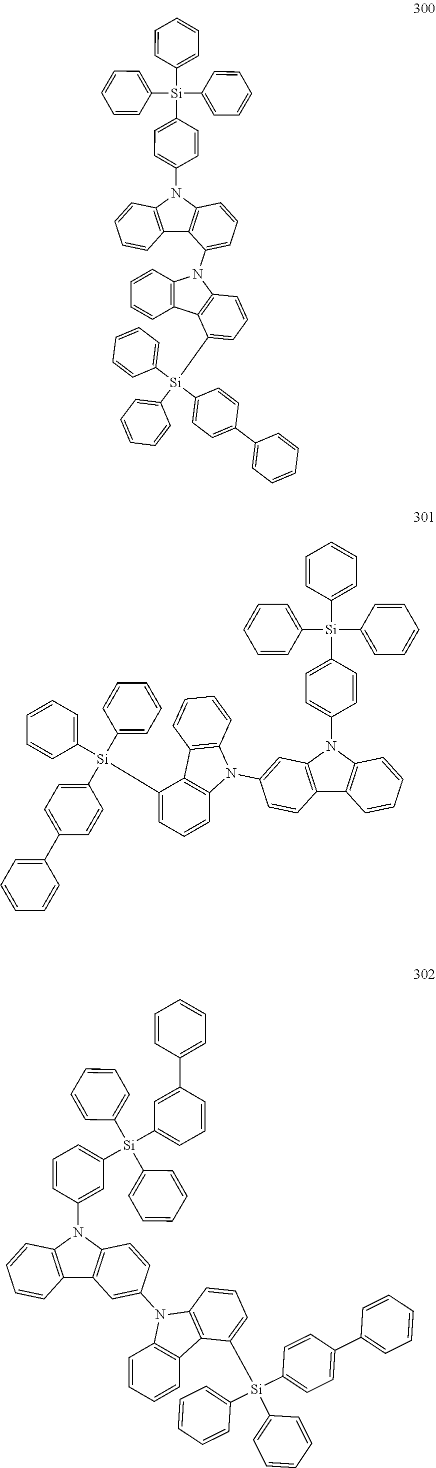 [SMILES: c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4ccccc4)cc3)cc2)cc1 ; c1ccc(-c2ccc([Si](c3ccccc3)(c3ccccc3)c3cccc4c3c3ccccc3n4-c3ccc4c5ccccc5n(-c5ccc([Si](c6ccccc6)(c6ccccc6)c6ccccc6)cc5)c4c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-c5ccccc5)c4)c3)cc2)cc1]

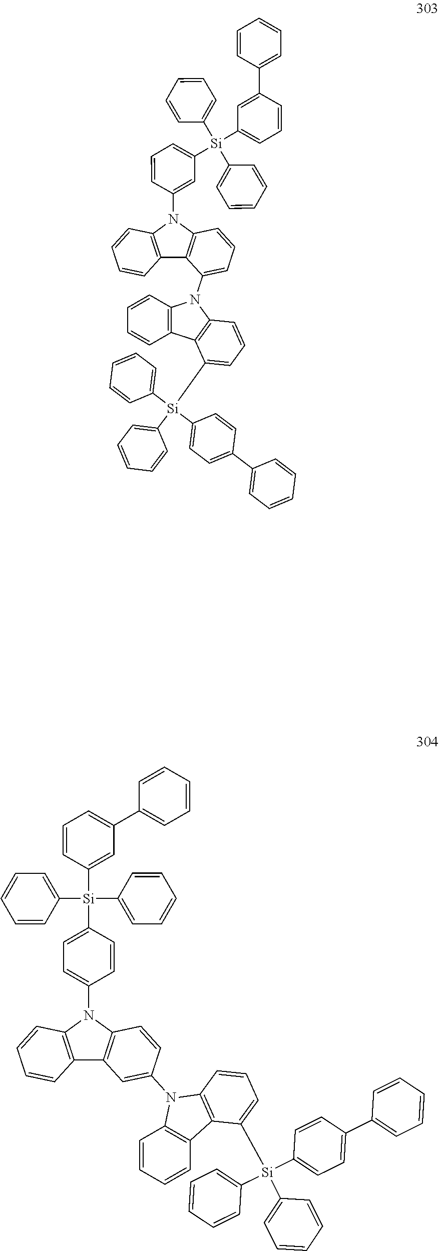 [SMILES: c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4cccc(-c5ccccc5)c4)c3)cc2)cc1 ; c1ccc(-c2ccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4cccc(-c5ccccc5)c4)cc3)cc2)cc1]

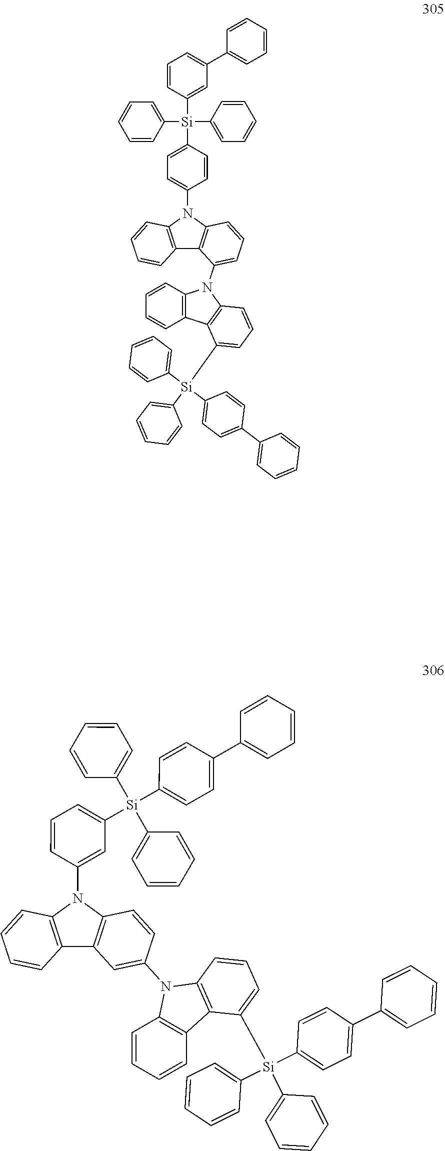 [SMILES: c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4cccc(-c5ccccc5)c4)cc3)cc2)cc1 ; c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccc(-c9ccccc9)cc8)cccc76)ccc54)c3)cc2)cc1]

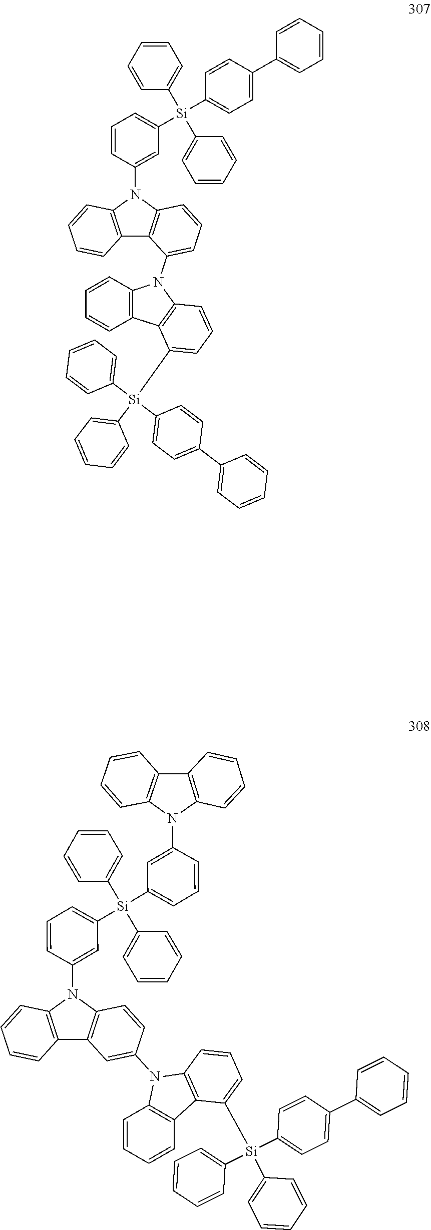 [SMILES: c1ccc(-c2ccc([Si](c3ccccc3)(c3ccccc3)c3cccc(-n4c5ccccc5c5c(-n6c7ccccc7c7c([Si](c8ccccc8)(c8ccccc8)c8ccc(-c9ccccc9)cc8)cccc76)cccc54)c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)cc2)cc1]

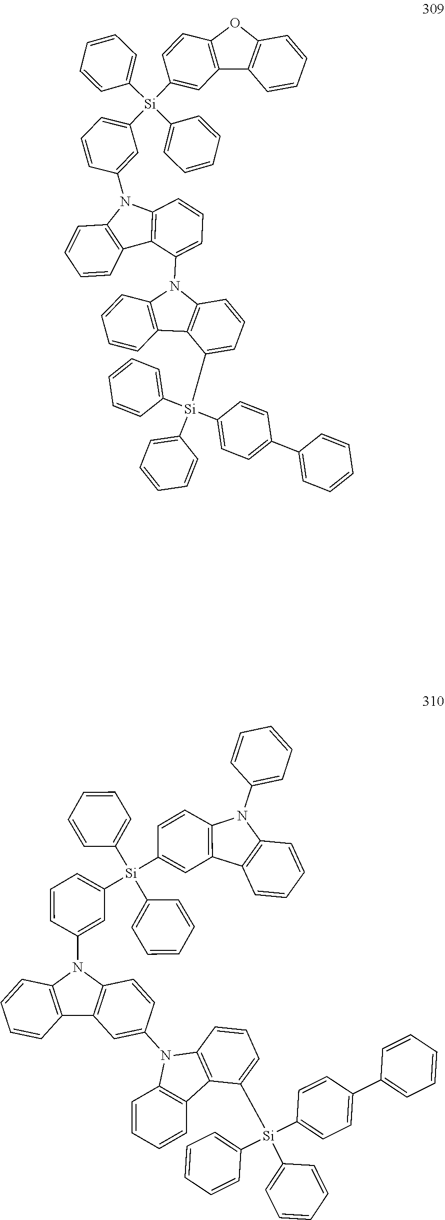 [SMILES: c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4ccc5oc6ccccc6c5c4)c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)cc2)cc1]

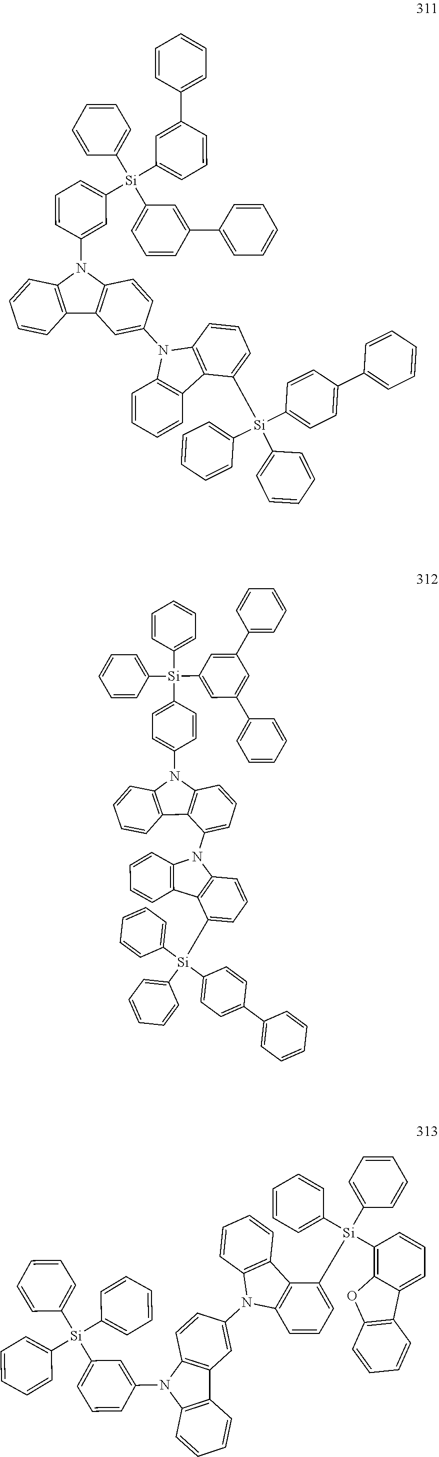 [SMILES: c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4cccc(-c5ccccc5)c4)c4cccc(-c5ccccc5)c4)c3)cc2)cc1 ; c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4cc(-c5ccccc5)cc(-c5ccccc5)c4)cc3)cc2)cc1 ; c1ccc([Si](c2ccccc2)(c2ccccc2)c2cccc(-n3c4ccccc4c4cc(-n5c6ccccc6c6c([Si](c7ccccc7)(c7ccccc7)c7cccc8c7oc7ccccc78)cccc65)ccc43)c2)cc1]

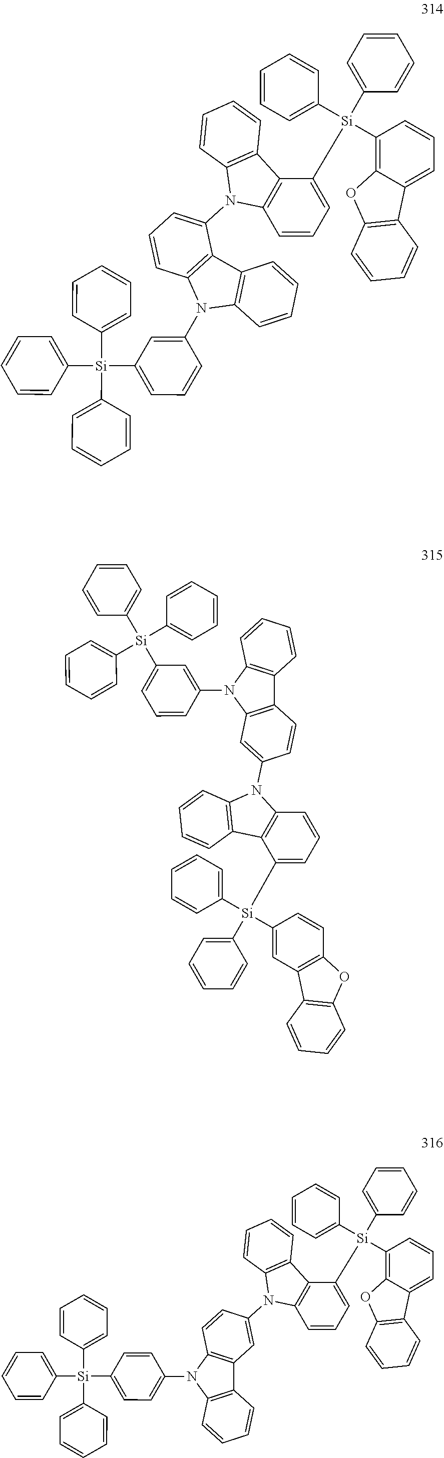 [SMILES: c1ccc([Si](c2ccccc2)(c2ccccc2)c2cccc(-n3c4ccccc4c4c(-n5c6ccccc6c6c([Si](c7ccccc7)(c7ccccc7)c7cccc8c7oc7ccccc78)cccc65)cccc43)c2)cc1 ; c1ccc([Si](c2ccccc2)(c2ccccc2)c2cccc(-n3c4ccccc4c4ccc(-n5c6ccccc6c6c([Si](c7ccccc7)(c7ccccc7)c7ccc8oc9ccccc9c8c7)cccc65)cc43)c2)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7cccc8c7oc7ccccc78)cccc65)ccc43)cc2)cc1]

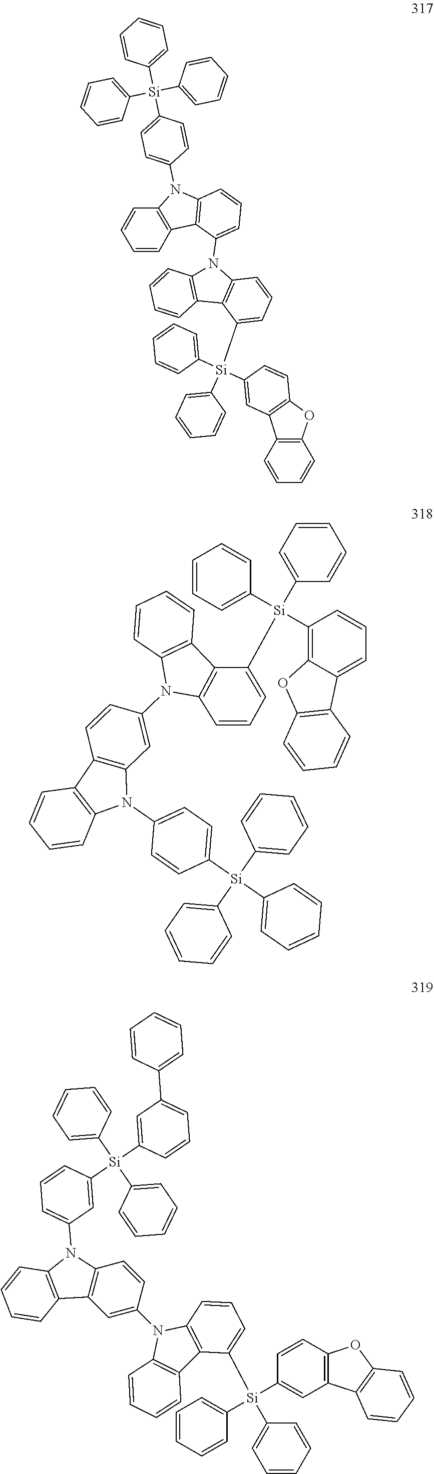 [SMILES: c1ccc([Si](c2ccccc2)(c2ccccc2)c2ccc(-n3c4ccccc4c4c(-n5c6ccccc6c6c([Si](c7ccccc7)(c7ccccc7)c7ccc8oc9ccccc9c8c7)cccc65)cccc43)cc2)cc1 ; c1ccc([Si](c2ccccc2)(c2ccccc2)c2ccc(-n3c4ccccc4c4ccc(-n5c6ccccc6c6c([Si](c7ccccc7)(c7ccccc7)c7cccc8c7oc7ccccc78)cccc65)cc43)cc2)cc1 ; c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8ccc9oc%10ccccc%10c9c8)cccc76)ccc54)c3)c2)cc1]

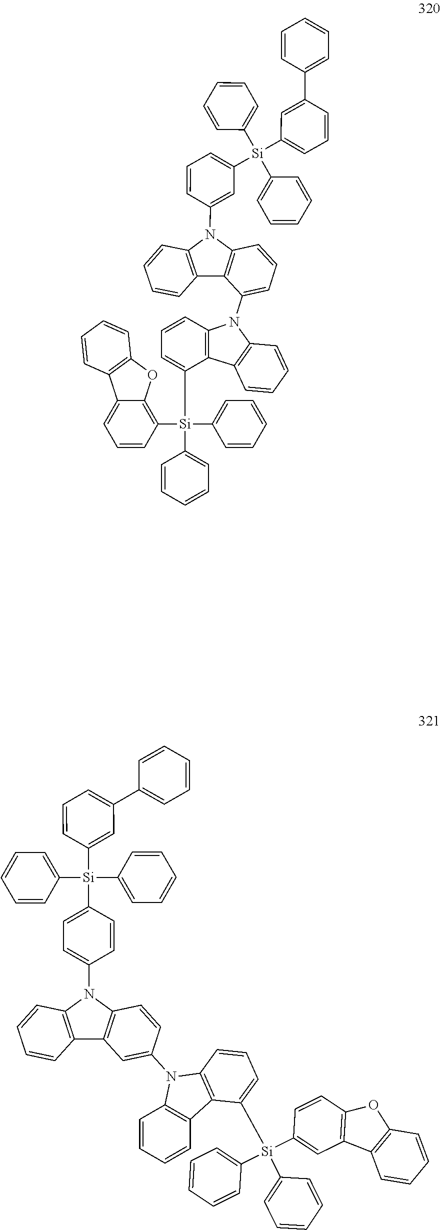 [SMILES: c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc9c8oc8ccccc89)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8ccc9oc%10ccccc%10c9c8)cccc76)ccc54)cc3)c2)cc1]

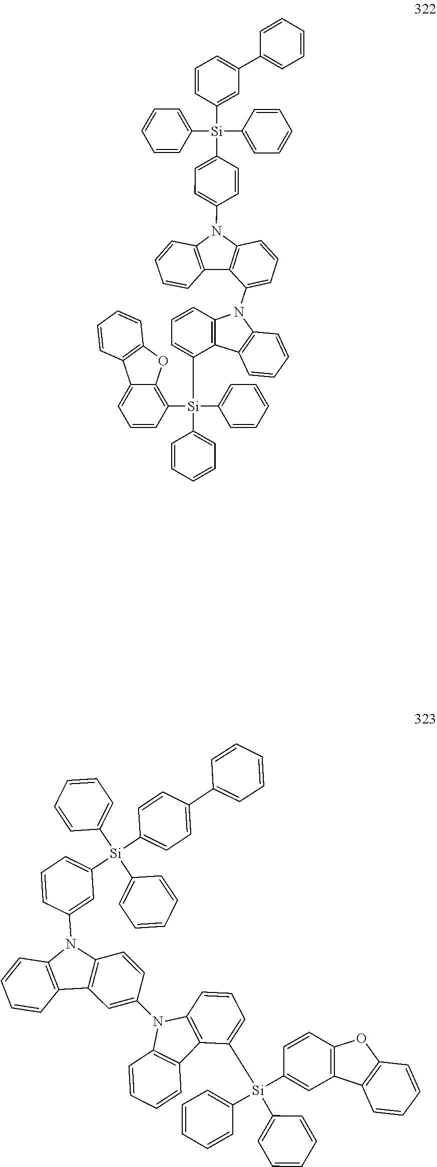 [SMILES: c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc9c8oc8ccccc89)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccc9oc%10ccccc%10c9c8)cccc76)ccc54)c3)cc2)cc1]

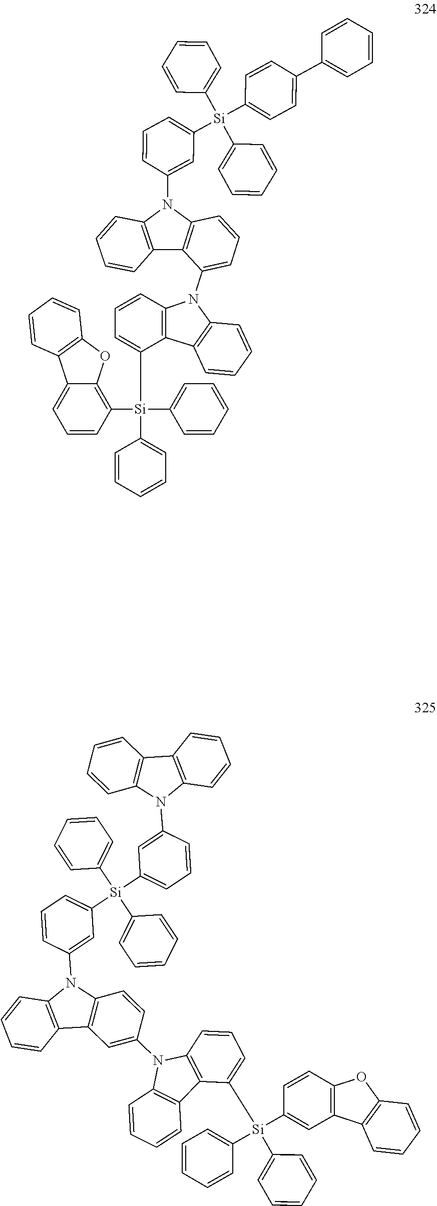 [SMILES: c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc9c8oc8ccccc89)cccc76)cccc54)c3)cc2)cc1 ; c1ccc([Si](c2ccccc2)(c2cccc(-n3c4ccccc4c4ccccc43)c2)c2cccc(-n3c4ccccc4c4cc(-n5c6ccccc6c6c([Si](c7ccccc7)(c7ccccc7)c7ccc8oc9ccccc9c8c7)cccc65)ccc43)c2)cc1]

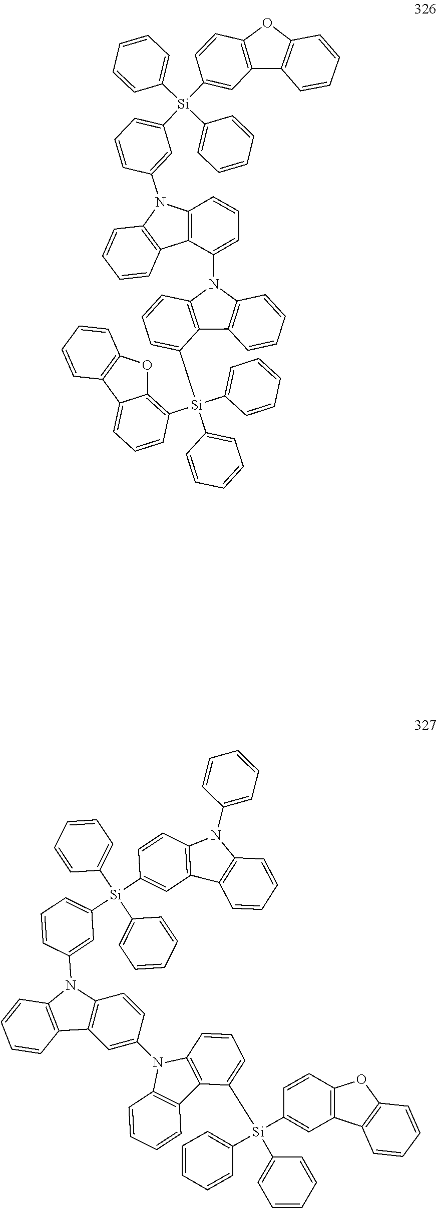 [SMILES: c1ccc([Si](c2ccccc2)(c2cccc(-n3c4ccccc4c4c(-n5c6ccccc6c6c([Si](c7ccccc7)(c7ccccc7)c7cccc8c7oc7ccccc78)cccc65)cccc43)c2)c2ccc3oc4ccccc4c3c2)cc1 ; c1ccc(-n2c3ccccc3c3cc([Si](c4ccccc4)(c4ccccc4)c4cccc(-n5c6ccccc6c6cc(-n7c8ccccc8c8c([Si](c9ccccc9)(c9ccccc9)c9ccc%10oc%11ccccc%11c%10c9)cccc87)ccc65)c4)ccc32)cc1]

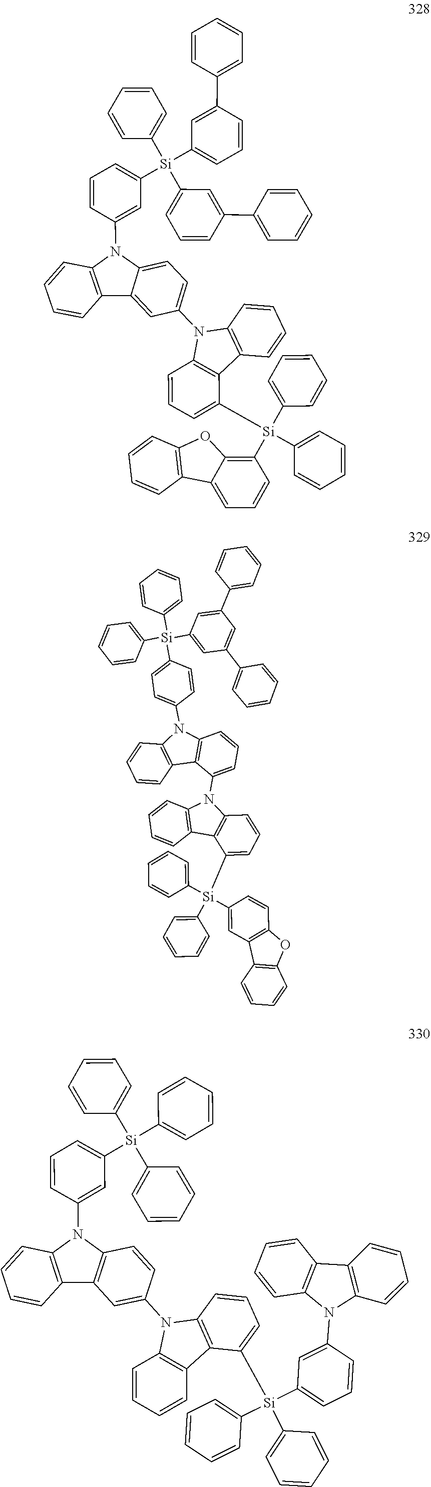

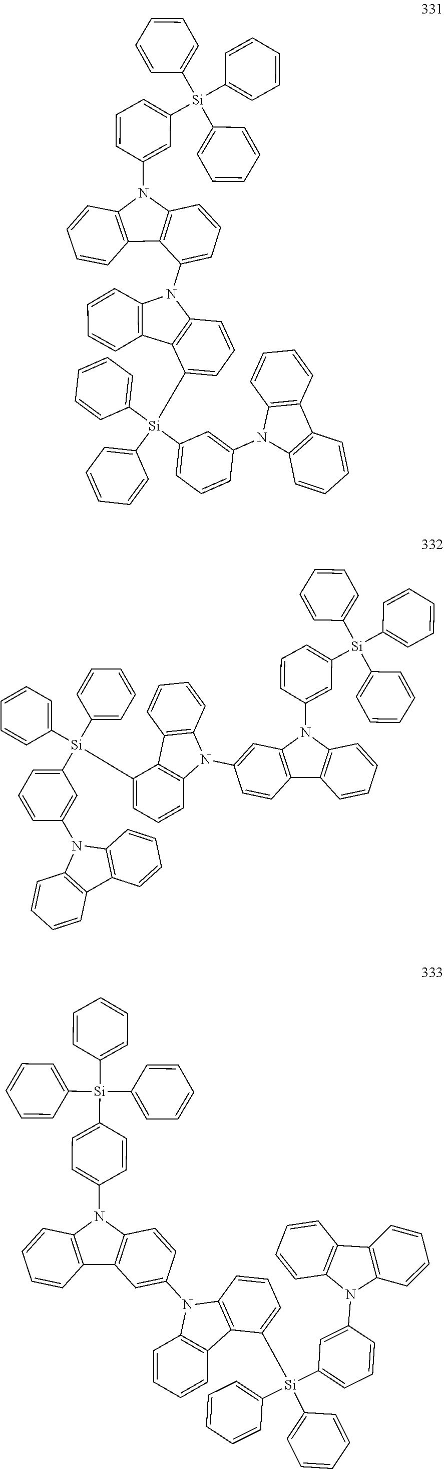

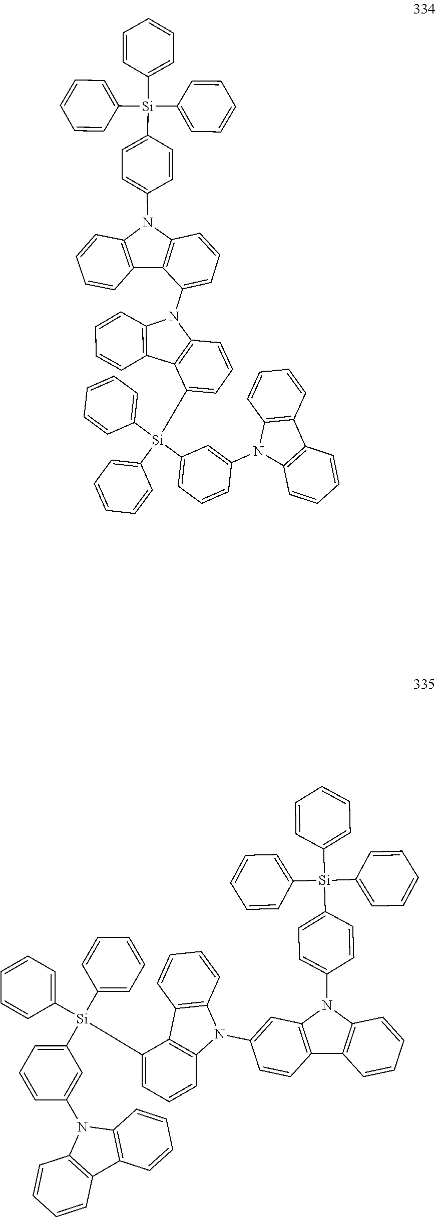

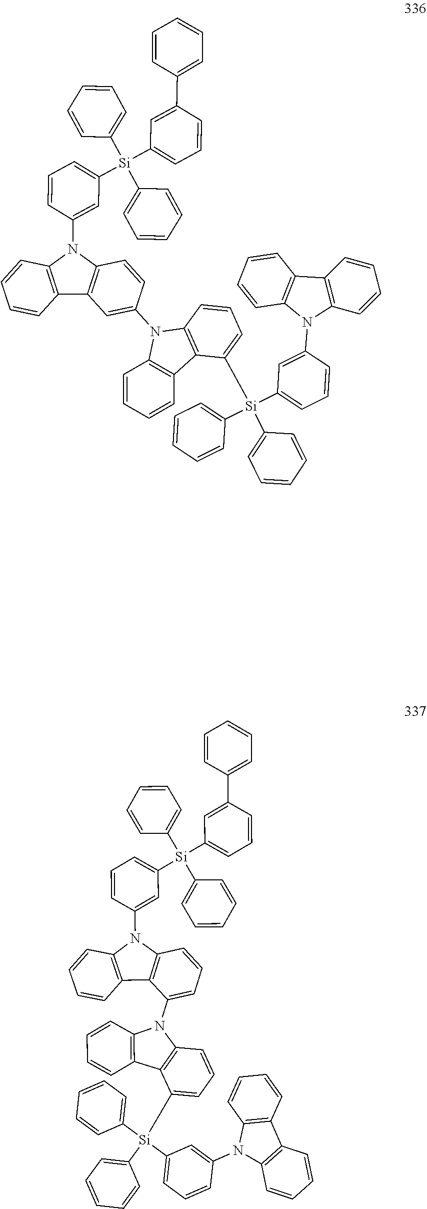 [SMILES: c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)c2)cc1 ; c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)c3)c2)cc1]

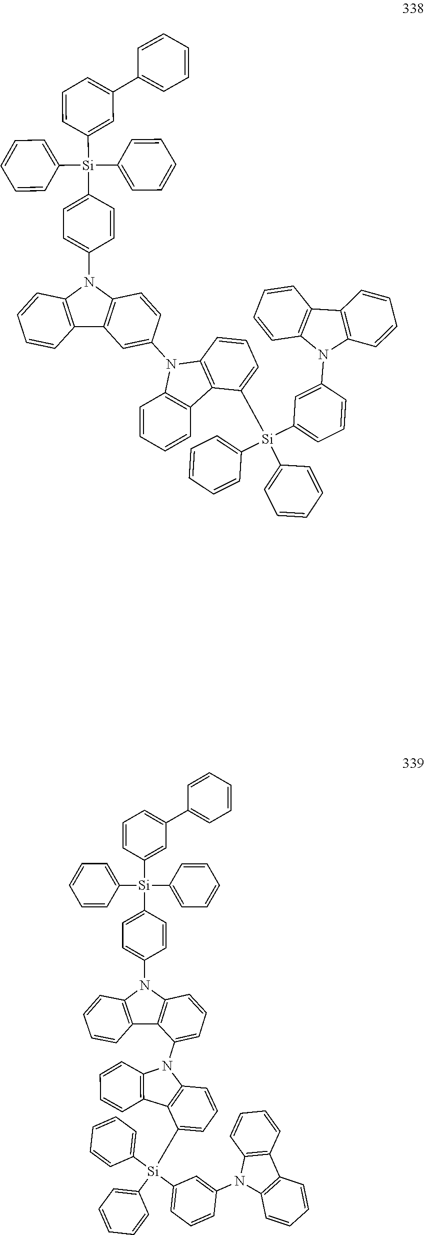 [SMILES: c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)cc3)c2)cc1 ; c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)cc3)c2)cc1]

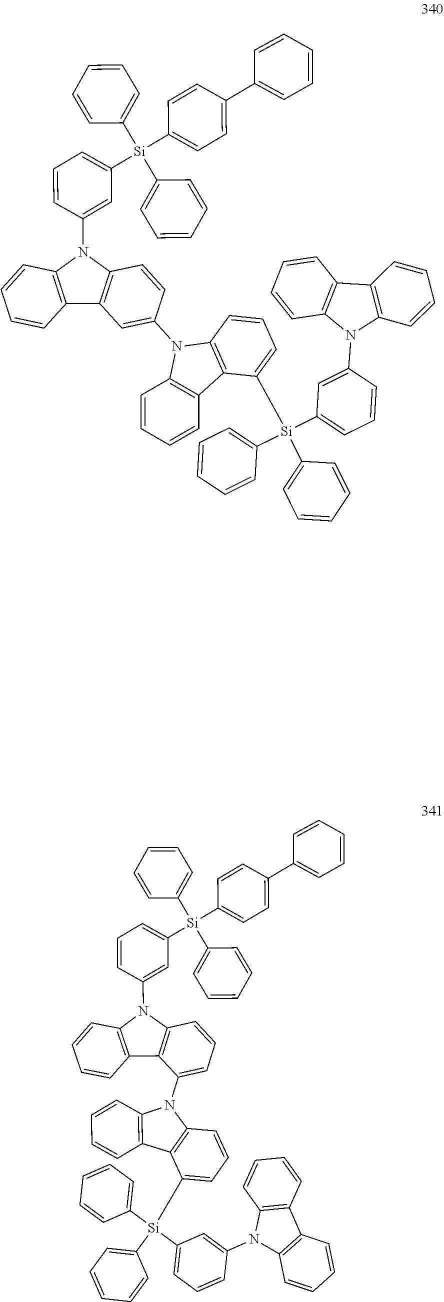 [SMILES: c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)c3)cc2)cc1]

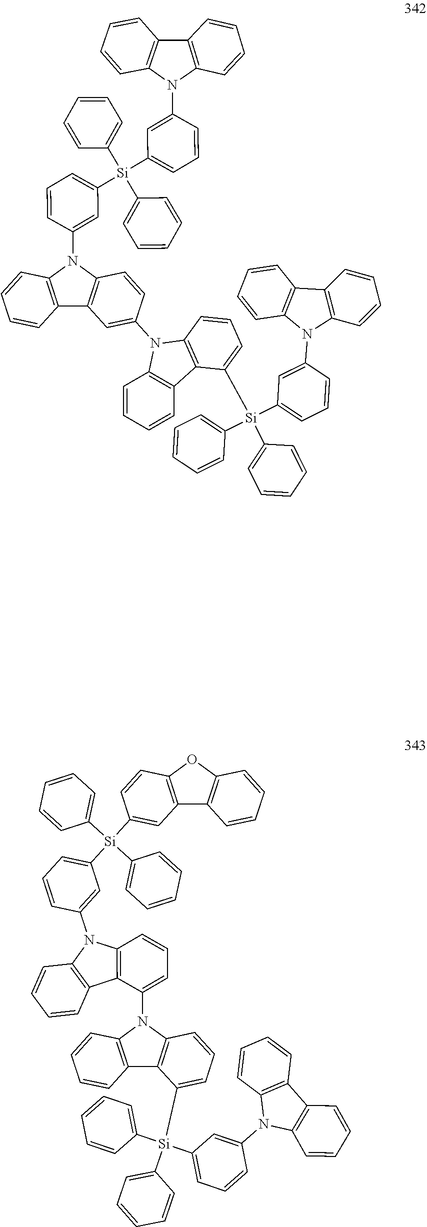 [SMILES: c1ccc([Si](c2ccccc2)(c2cccc(-n3c4ccccc4c4ccccc43)c2)c2cccc(-n3c4ccccc4c4cc(-n5c6ccccc6c6c([Si](c7ccccc7)(c7ccccc7)c7cccc(-n8c9ccccc9c9ccccc98)c7)cccc65)ccc43)c2)cc1 ; c1ccc([Si](c2ccccc2)(c2cccc(-n3c4ccccc4c4c(-n5c6ccccc6c6c([Si](c7ccccc7)(c7ccccc7)c7cccc(-n8c9ccccc9c9ccccc98)c7)cccc65)cccc43)c2)c2ccc3oc4ccccc4c3c2)cc1]

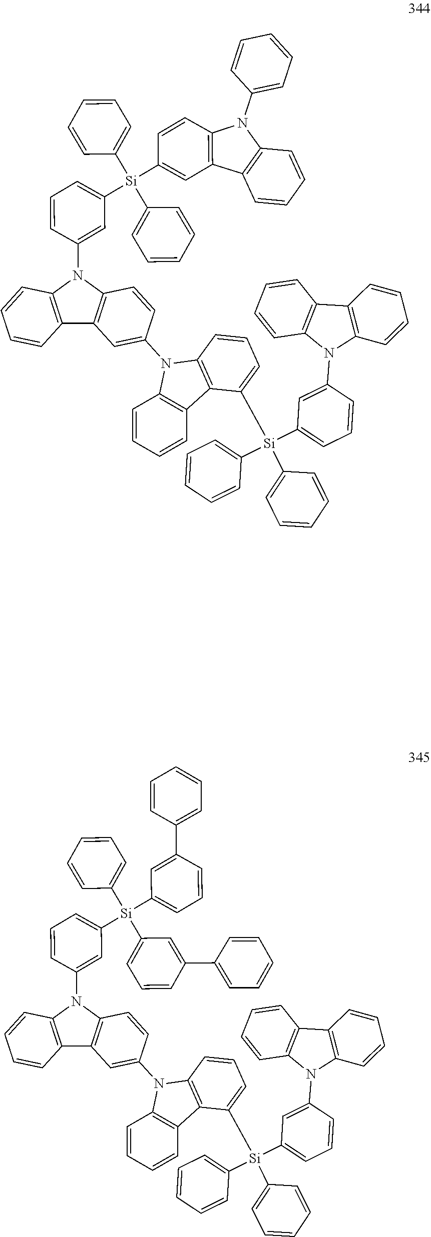 [SMILES: c1ccc(-n2c3ccccc3c3cc([Si](c4ccccc4)(c4ccccc4)c4cccc(-n5c6ccccc6c6cc(-n7c8ccccc8c8c([Si](c9ccccc9)(c9ccccc9)c9cccc(-n%10c%11ccccc%11c%11ccccc%11%10)c9)cccc87)ccc65)c4)ccc32)cc1 ; c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)c2)cc1]

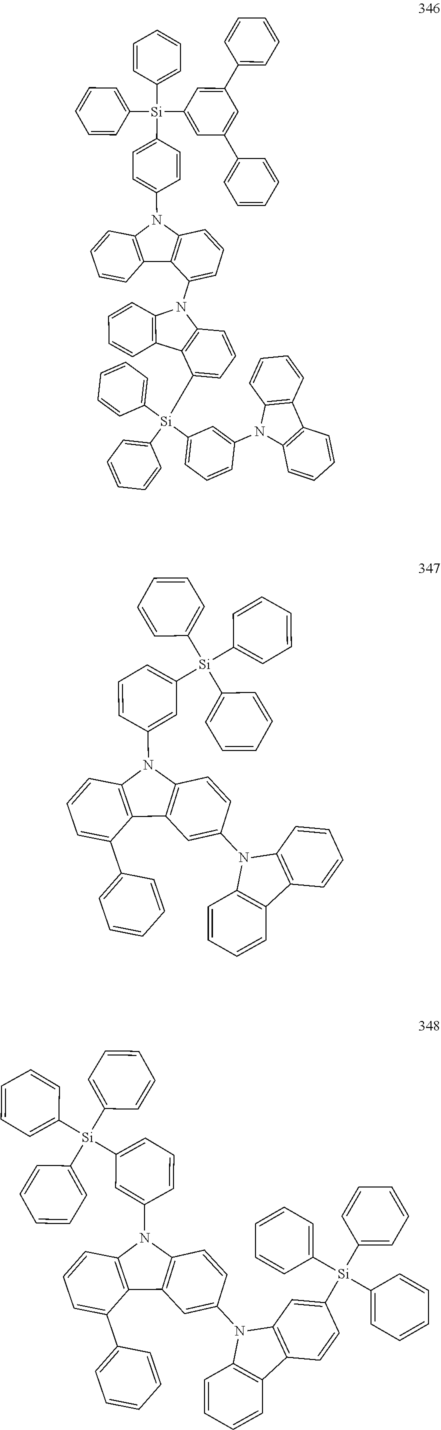 [SMILES: c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)cc3)c2)cc1 ; c1ccc(-c2cccc3c2c2cc(-n4c5ccccc5c5ccccc54)ccc2n3-c2cccc([Si](c3ccccc3)(c3ccccc3)c3ccccc3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccccc3)c2)cc1]

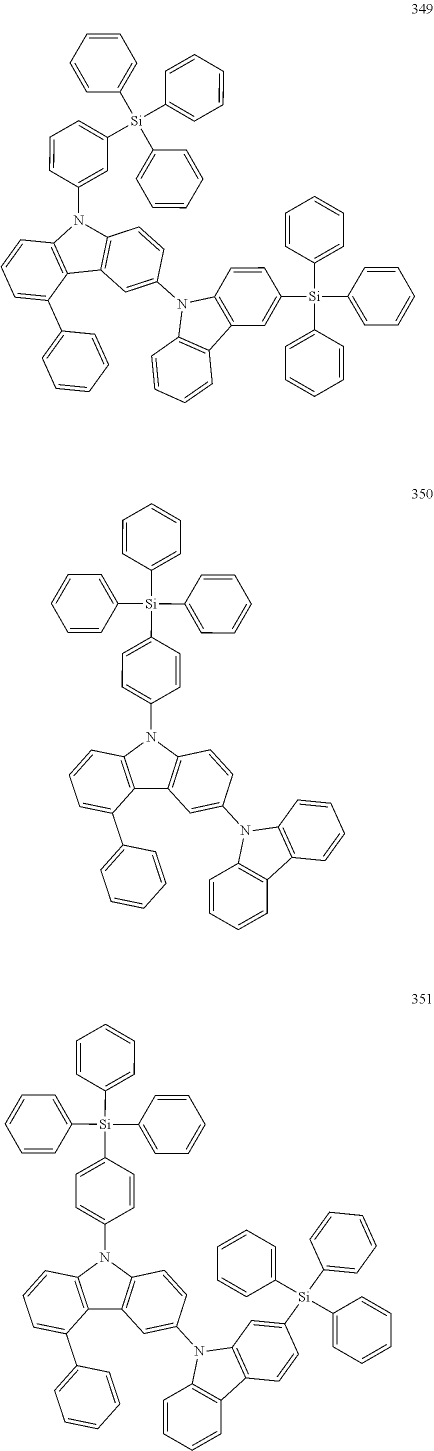 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3ccccc3)c2)cc1 ; c1ccc(-c2cccc3c2c2cc(-n4c5ccccc5c5ccccc54)ccc2n3-c2ccc([Si](c3ccccc3)(c3ccccc3)c3ccccc3)cc2)cc1 ; c1ccc(-c2cccc3c2c2cc(-n4c5ccccc5c5ccc([Si](c6ccccc6)(c6ccccc6)c6ccccc6)cc54)ccc2n3-c2ccc([Si](c3ccccc3)(c3ccccc3)c3ccccc3)cc2)cc1]

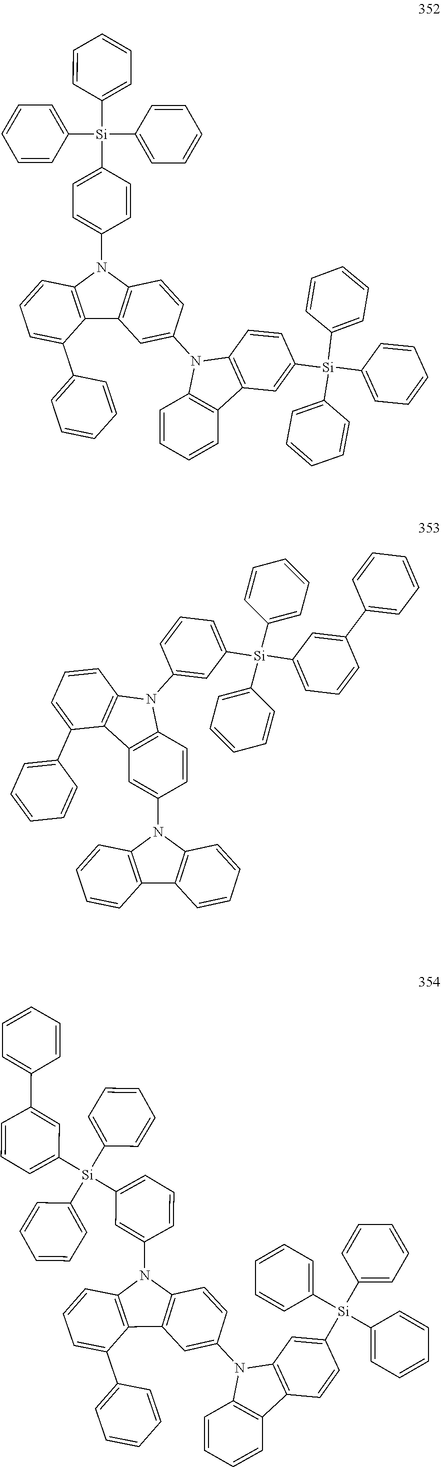 [SMILES: c1ccc(-c2cccc3c2c2cc(-n4c5ccccc5c5cc([Si](c6ccccc6)(c6ccccc6)c6ccccc6)ccc54)ccc2n3-c2ccc([Si](c3ccccc3)(c3ccccc3)c3ccccc3)cc2)cc1 ; c1ccc(-c2cccc([Si](c3ccccc3)(c3ccccc3)c3cccc(-n4c5ccc(-n6c7ccccc7c7ccccc76)cc5c5c(-c6ccccc6)cccc54)c3)c2)cc1 ; c1ccc(-c2cccc([Si](c3ccccc3)(c3ccccc3)c3cccc(-n4c5ccc(-n6c7ccccc7c7ccc([Si](c8ccccc8)(c8ccccc8)c8ccccc8)cc76)cc5c5c(-c6ccccc6)cccc54)c3)c2)cc1]

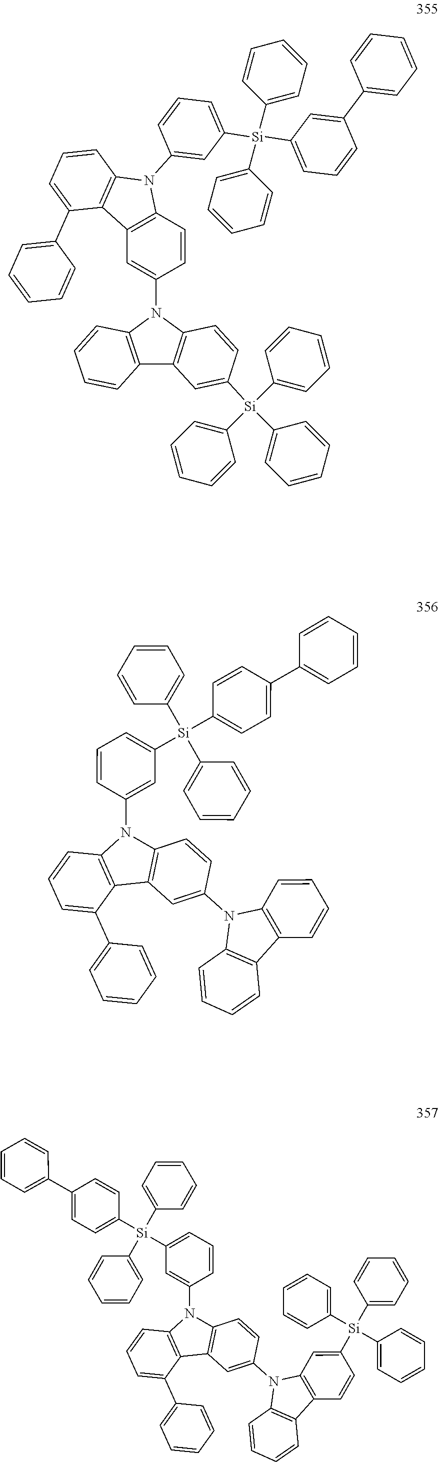 [SMILES: c1ccc(-c2cccc([Si](c3ccccc3)(c3ccccc3)c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1 ; c1ccc(-c2ccc([Si](c3ccccc3)(c3ccccc3)c3cccc(-n4c5ccc(-n6c7ccccc7c7ccccc76)cc5c5c(-c6ccccc6)cccc54)c3)cc2)cc1 ; c1ccc(-c2ccc([Si](c3ccccc3)(c3ccccc3)c3cccc(-n4c5ccc(-n6c7ccccc7c7ccc([Si](c8ccccc8)(c8ccccc8)c8ccccc8)cc76)cc5c5c(-c6ccccc6)cccc54)c3)cc2)cc1]

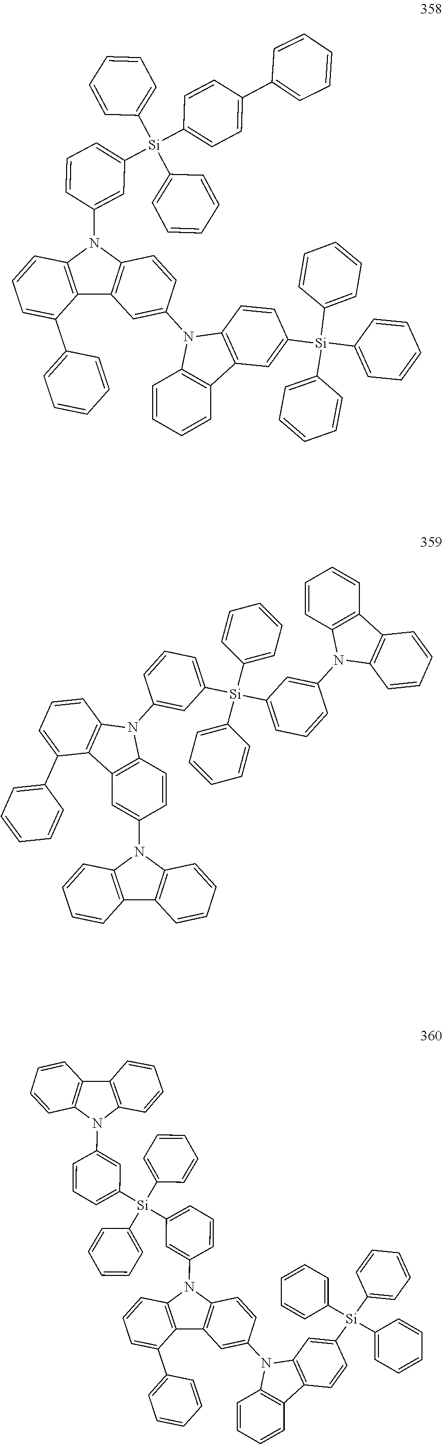

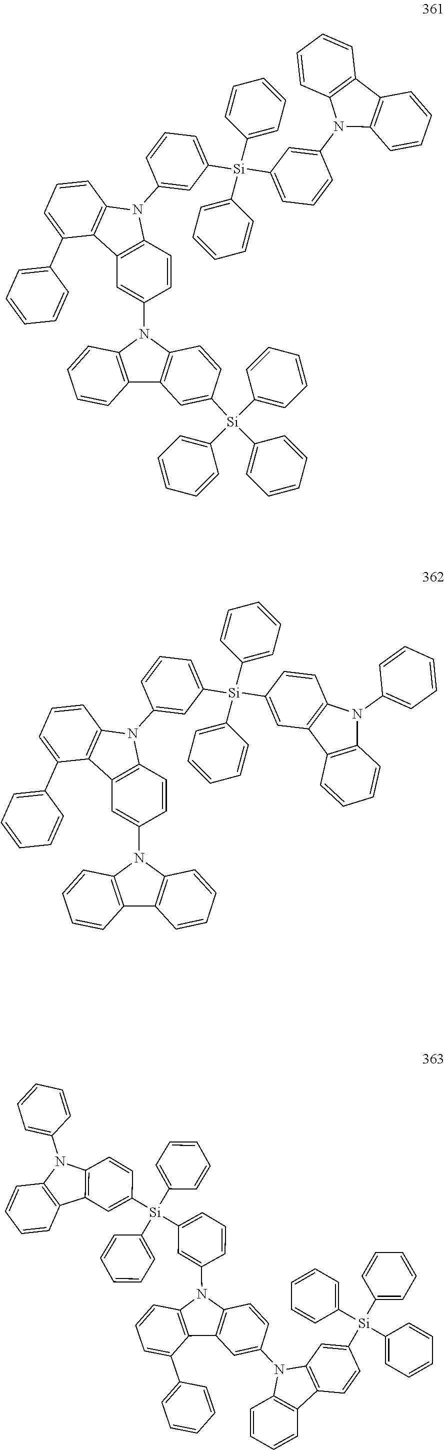 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2cc(-n4c5ccccc5c5ccccc54)ccc2n3-c2cccc([Si](c3ccccc3)(c3ccccc3)c3ccc4c(c3)c3ccccc3n4-c3ccccc3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccc4c(c3)c3ccccc3n4-c3ccccc3)c2)cc1]

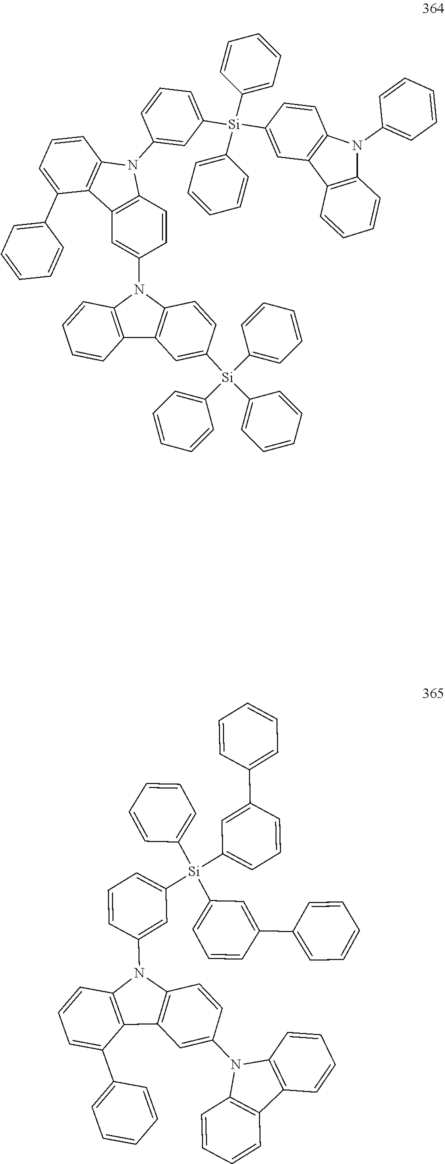 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3ccc4c(c3)c3ccccc3n4-c3ccccc3)c2)cc1 ; c1ccc(-c2cccc([Si](c3ccccc3)(c3cccc(-c4ccccc4)c3)c3cccc(-n4c5ccc(-n6c7ccccc7c7ccccc76)cc5c5c(-c6ccccc6)cccc54)c3)c2)cc1]

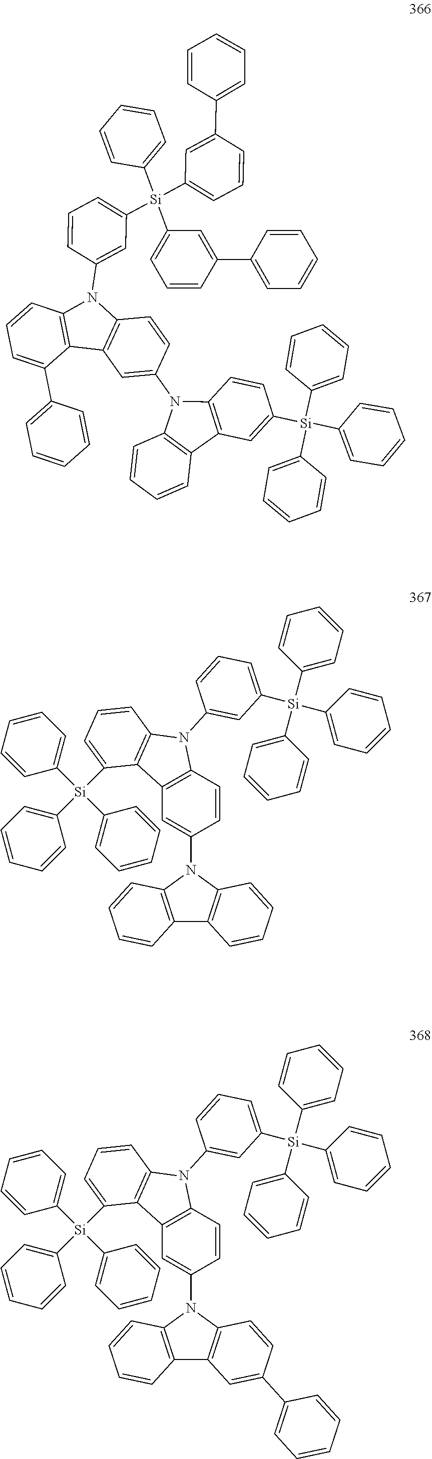 [SMILES: c1ccc(-c2cccc([Si](c3ccccc3)(c3cccc(-c4ccccc4)c3)c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1 ; c1ccc([Si](c2ccccc2)(c2ccccc2)c2cccc(-n3c4ccc(-n5c6ccccc6c6ccccc65)cc4c4c([Si](c5ccccc5)(c5ccccc5)c5ccccc5)cccc43)c2)cc1 ; c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)cc1]

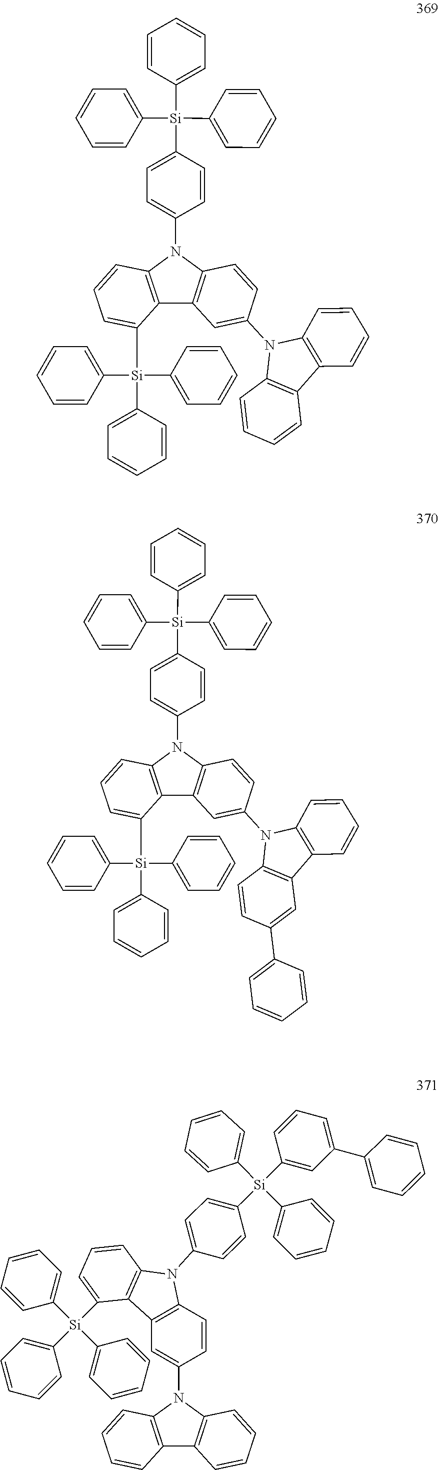 [SMILES: c1ccc([Si](c2ccccc2)(c2ccccc2)c2ccc(-n3c4ccc(-n5c6ccccc6c6ccccc65)cc4c4c([Si](c5ccccc5)(c5ccccc5)c5ccccc5)cccc43)cc2)cc1 ; c1ccc(-c2ccc3c(c2)c2ccccc2n3-c2ccc3c(c2)c2c([Si](c4ccccc4)(c4ccccc4)c4ccccc4)cccc2n3-c2ccc([Si](c3ccccc3)(c3ccccc3)c3ccccc3)cc2)cc1 ; c1ccc(-c2cccc([Si](c3ccccc3)(c3ccccc3)c3ccc(-n4c5ccc(-n6c7ccccc7c7ccccc76)cc5c5c([Si](c6ccccc6)(c6ccccc6)c6ccccc6)cccc54)cc3)c2)cc1]

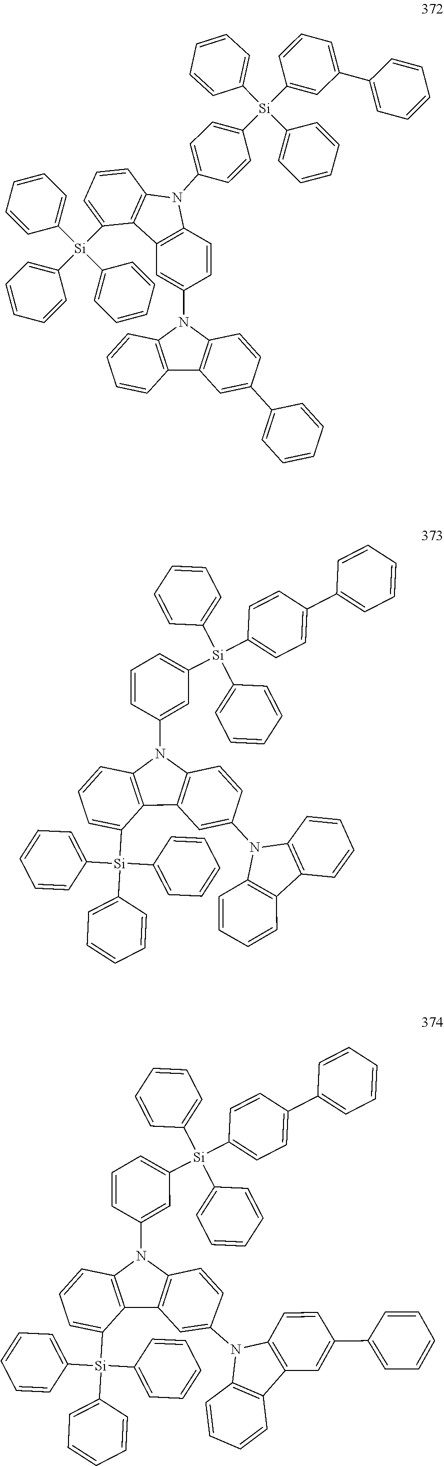 [SMILES: c1ccc(-c2cccc([Si](c3ccccc3)(c3ccccc3)c3ccc(-n4c5ccc(-n6c7ccccc7c7cc(-c8ccccc8)ccc76)cc5c5c([Si](c6ccccc6)(c6ccccc6)c6ccccc6)cccc54)cc3)c2)cc1 ; c1ccc(-c2ccc([Si](c3ccccc3)(c3ccccc3)c3cccc(-n4c5ccc(-n6c7ccccc7c7ccccc76)cc5c5c([Si](c6ccccc6)(c6ccccc6)c6ccccc6)cccc54)c3)cc2)cc1 ; c1ccc(-c2ccc([Si](c3ccccc3)(c3ccccc3)c3cccc(-n4c5ccc(-n6c7ccccc7c7cc(-c8ccccc8)ccc76)cc5c5c([Si](c6ccccc6)(c6ccccc6)c6ccccc6)cccc54)c3)cc2)cc1]

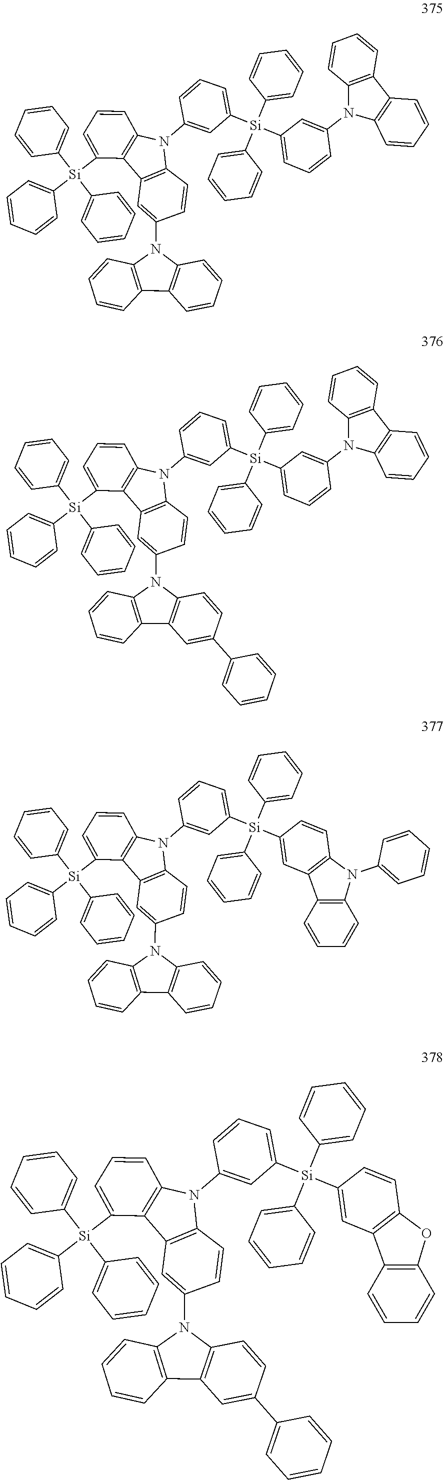

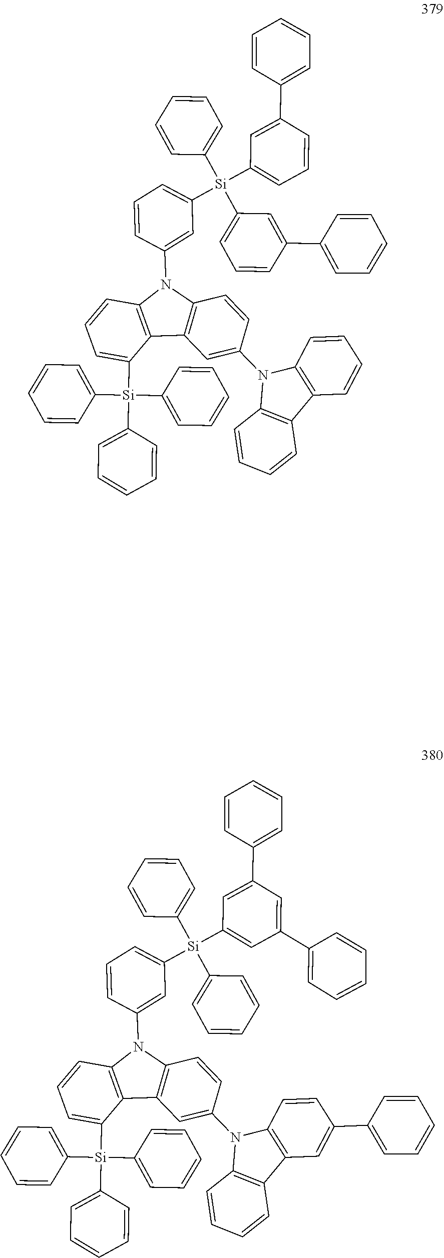 [SMILES: c1ccc(-c2cccc([Si](c3ccccc3)(c3cccc(-c4ccccc4)c3)c3cccc(-n4c5ccc(-n6c7ccccc7c7ccccc76)cc5c5c([Si](c6ccccc6)(c6ccccc6)c6ccccc6)cccc54)c3)c2)cc1 ; c1ccc(-c2cc(-c3ccccc3)cc([Si](c3ccccc3)(c3ccccc3)c3cccc(-n4c5ccc(-n6c7ccccc7c7cc(-c8ccccc8)ccc76)cc5c5c([Si](c6ccccc6)(c6ccccc6)c6ccccc6)cccc54)c3)c2)cc1]

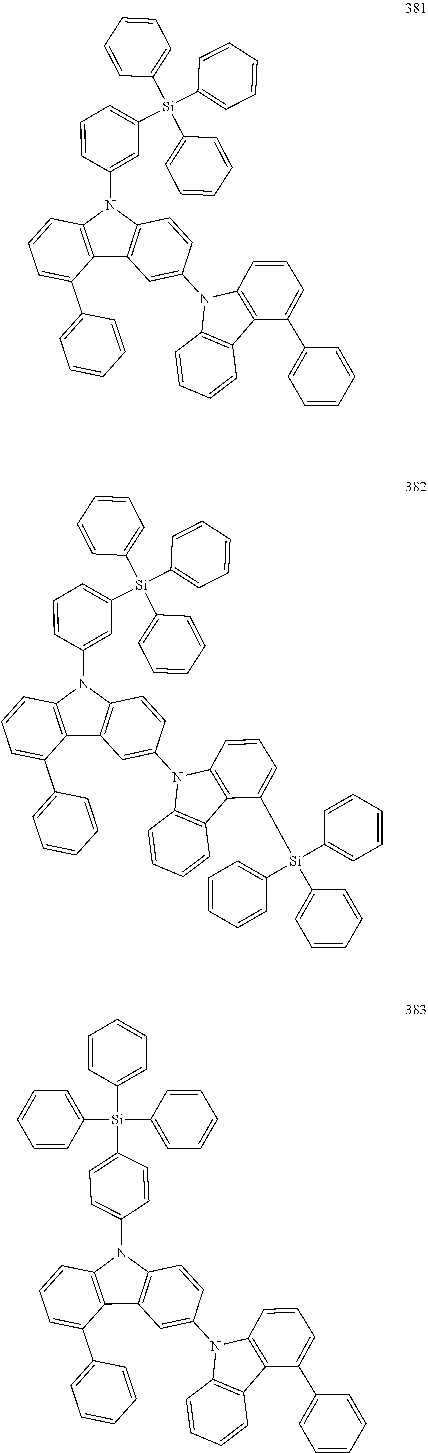 [SMILES: c1ccc(-c2cccc3c2c2ccccc2n3-c2ccc3c(c2)c2c(-c4ccccc4)cccc2n3-c2cccc([Si](c3ccccc3)(c3ccccc3)c3ccccc3)c2)cc1 ; c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3ccccc3)c2)cc1 ; c1ccc(-c2cccc3c2c2ccccc2n3-c2ccc3c(c2)c2c(-c4ccccc4)cccc2n3-c2ccc([Si](c3ccccc3)(c3ccccc3)c3ccccc3)cc2)cc1]

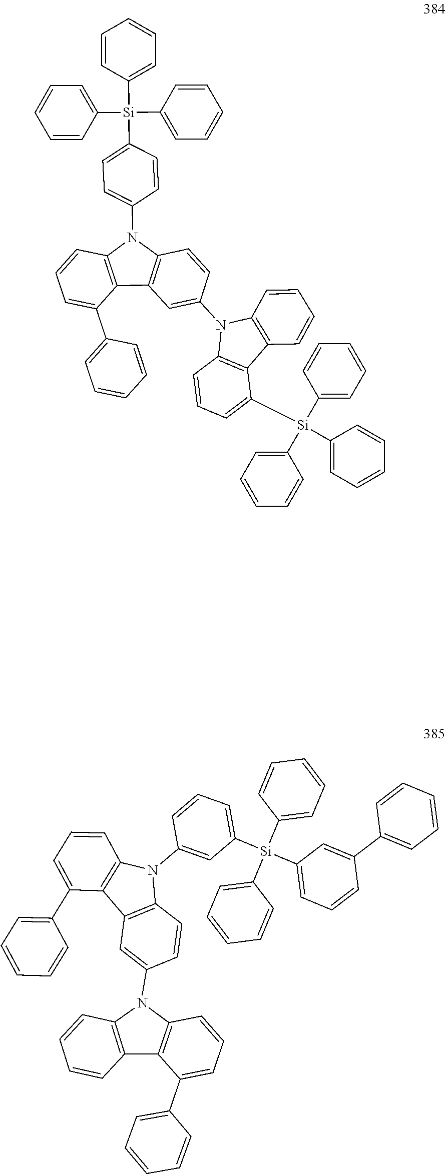 [SMILES: c1ccc(-c2cccc3c2c2cc(-n4c5ccccc5c5c([Si](c6ccccc6)(c6ccccc6)c6ccccc6)cccc54)ccc2n3-c2ccc([Si](c3ccccc3)(c3ccccc3)c3ccccc3)cc2)cc1 ; c1ccc(-c2cccc([Si](c3ccccc3)(c3ccccc3)c3cccc(-n4c5ccc(-n6c7ccccc7c7c(-c8ccccc8)cccc76)cc5c5c(-c6ccccc6)cccc54)c3)c2)cc1]

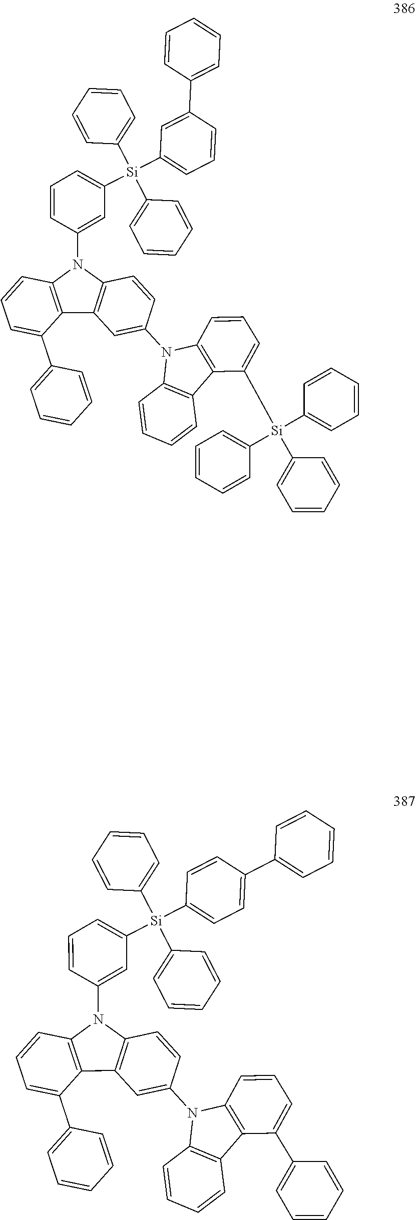 [SMILES: c1ccc(-c2cccc([Si](c3ccccc3)(c3ccccc3)c3cccc(-n4c5ccc(-n6c7ccccc7c7c([Si](c8ccccc8)(c8ccccc8)c8ccccc8)cccc76)cc5c5c(-c6ccccc6)cccc54)c3)c2)cc1 ; c1ccc(-c2ccc([Si](c3ccccc3)(c3ccccc3)c3cccc(-n4c5ccc(-n6c7ccccc7c7c(-c8ccccc8)cccc76)cc5c5c(-c6ccccc6)cccc54)c3)cc2)cc1]

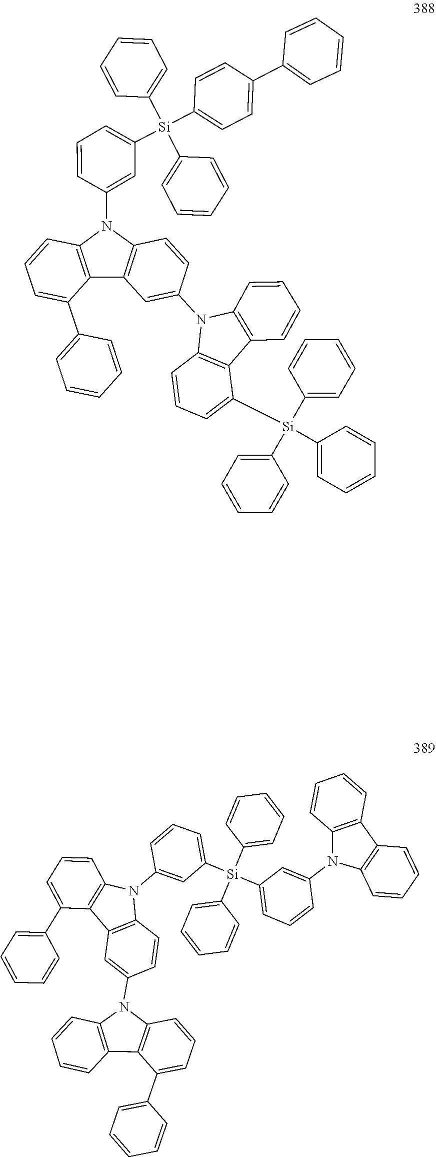 [SMILES: c1ccc(-c2ccc([Si](c3ccccc3)(c3ccccc3)c3cccc(-n4c5ccc(-n6c7ccccc7c7c([Si](c8ccccc8)(c8ccccc8)c8ccccc8)cccc76)cc5c5c(-c6ccccc6)cccc54)c3)cc2)cc1 ; c1ccc(-c2cccc3c2c2ccccc2n3-c2ccc3c(c2)c2c(-c4ccccc4)cccc2n3-c2cccc([Si](c3ccccc3)(c3ccccc3)c3cccc(-n4c5ccccc5c5ccccc54)c3)c2)cc1]

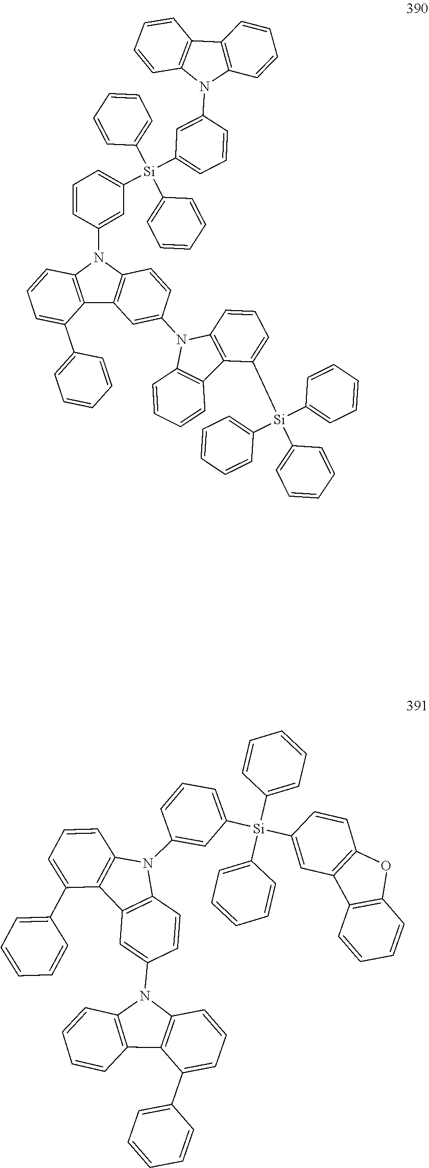 [SMILES: c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2ccccc2n3-c2ccc3c(c2)c2c(-c4ccccc4)cccc2n3-c2cccc([Si](c3ccccc3)(c3ccccc3)c3ccc4oc5ccccc5c4c3)c2)cc1]

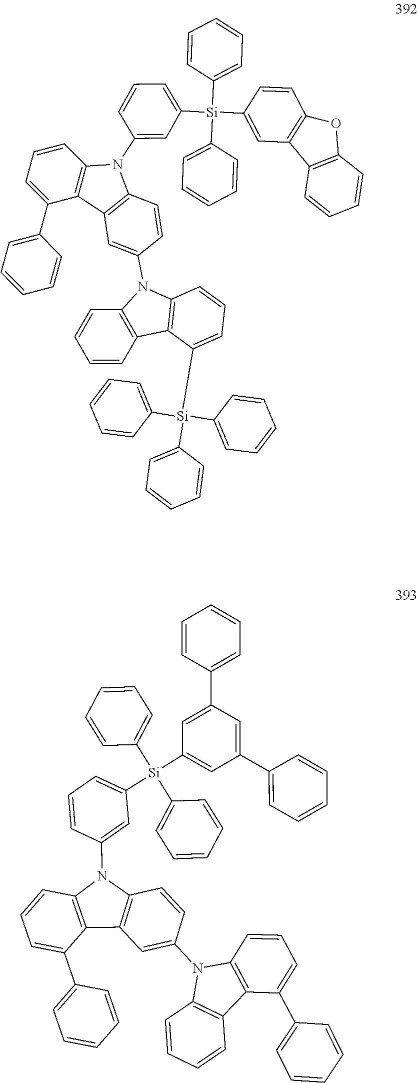 [SMILES: c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3ccc4oc5ccccc5c4c3)c2)cc1 ; c1ccc(-c2cc(-c3ccccc3)cc([Si](c3ccccc3)(c3ccccc3)c3cccc(-n4c5ccc(-n6c7ccccc7c7c(-c8ccccc8)cccc76)cc5c5c(-c6ccccc6)cccc54)c3)c2)cc1]

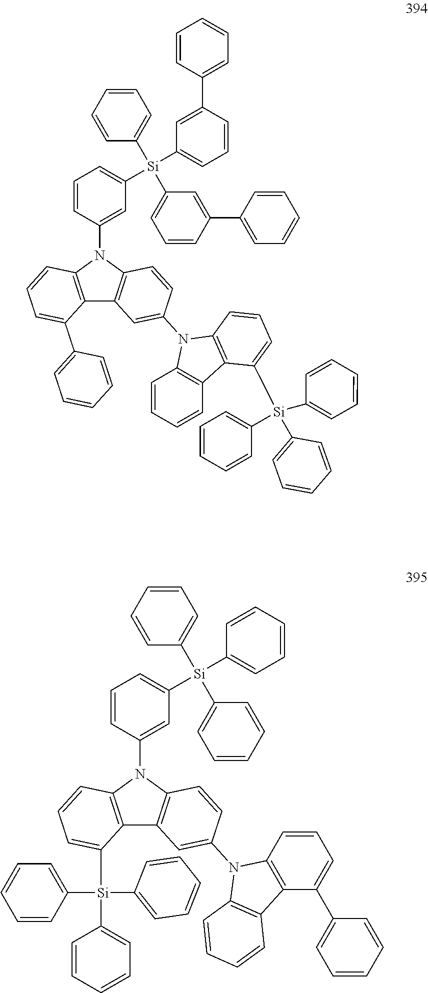 [SMILES: c1ccc(-c2cccc([Si](c3ccccc3)(c3cccc(-c4ccccc4)c3)c3cccc(-n4c5ccc(-n6c7ccccc7c7c([Si](c8ccccc8)(c8ccccc8)c8ccccc8)cccc76)cc5c5c(-c6ccccc6)cccc54)c3)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)cc1]

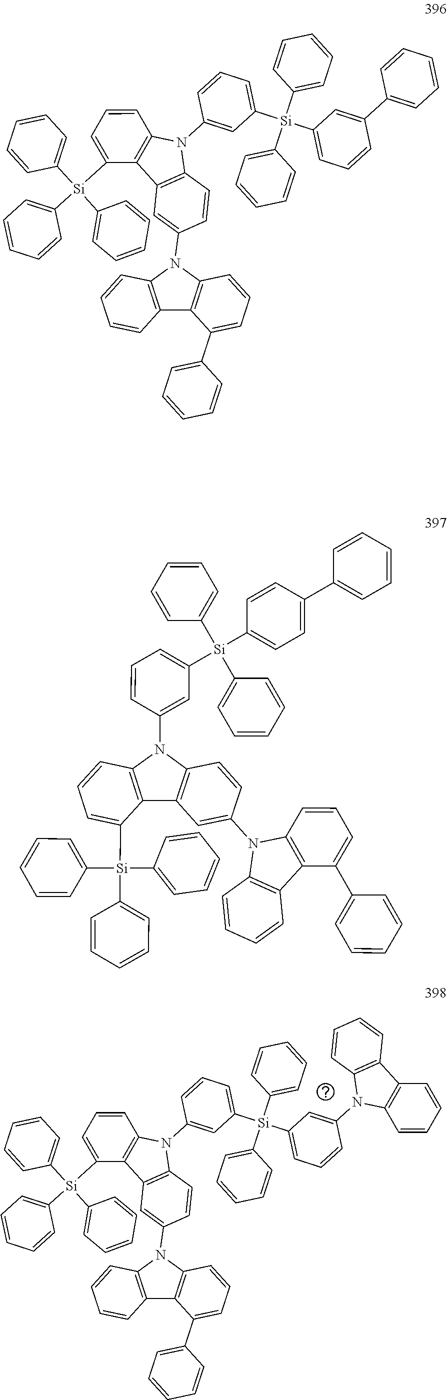 [SMILES: c1ccc(-c2cccc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1 ; c1ccc(-c2ccc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)cc2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3cccc(-n4c5ccccc5c5ccccc54)c3)c2)cc1]

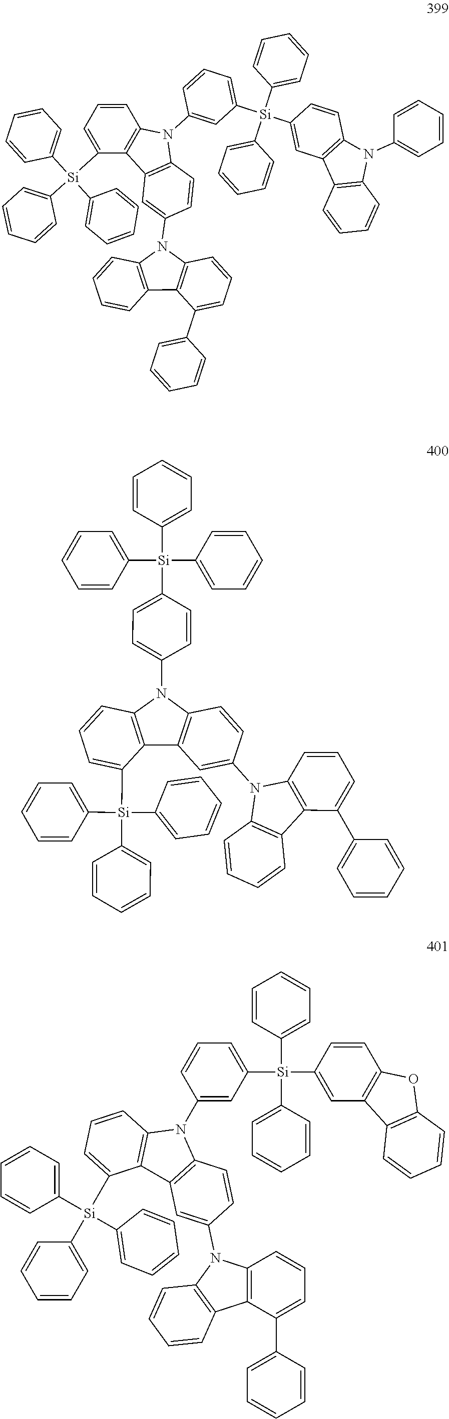 [SMILES: c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4c(c3)c3ccccc3n4-c3ccccc3)c2)cc1 ; c1ccc(-c2cccc3c2c2ccccc2n3-c2ccc3c(c2)c2c([Si](c4ccccc4)(c4ccccc4)c4ccccc4)cccc2n3-c2ccc([Si](c3ccccc3)(c3ccccc3)c3ccccc3)cc2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4oc5ccccc5c4c3)c2)cc1]

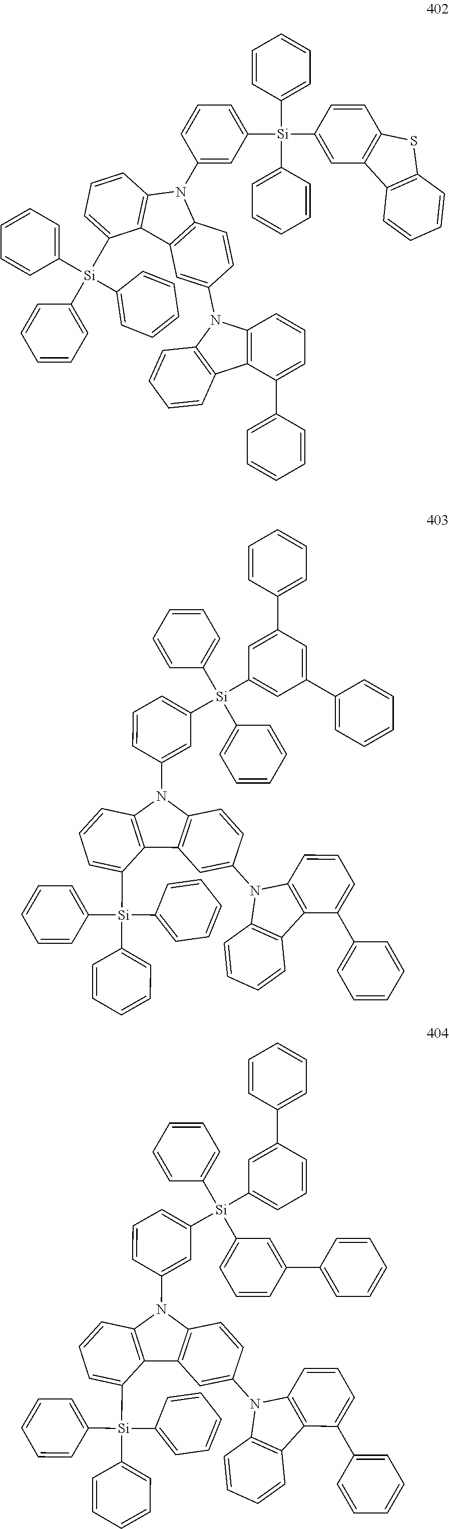 [SMILES: c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4sc5ccccc5c4c3)c2)cc1 ; c1ccc(-c2cc(-c3ccccc3)cc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1 ; c1ccc(-c2cccc([Si](c3ccccc3)(c3cccc(-c4ccccc4)c3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1]

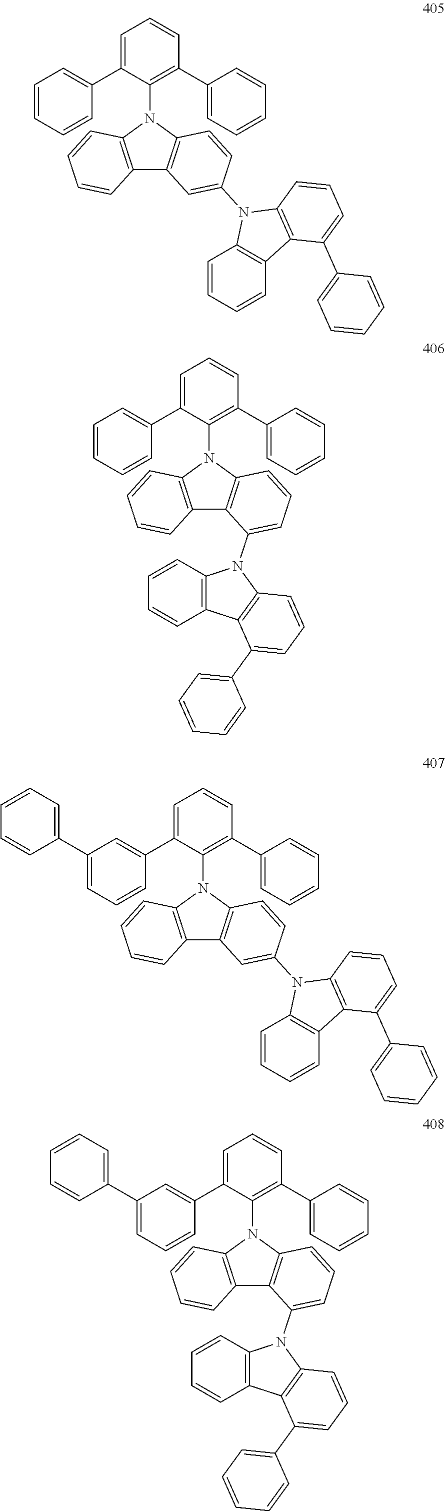 [SMILES: c1ccc(-c2cccc(-c3ccccc3)c2-n2c3ccccc3c3cc(-n4c5ccccc5c5c(-c6ccccc6)cccc54)ccc32)cc1 ; c1ccc(-c2cccc(-c3ccccc3)c2-n2c3ccccc3c3c(-n4c5ccccc5c5c(-c6ccccc6)cccc54)cccc32)cc1 ; c1ccc(-c2cccc(-c3cccc(-c4ccccc4)c3-n3c4ccccc4c4cc(-n5c6ccccc6c6c(-c7ccccc7)cccc65)ccc43)c2)cc1 ; c1ccc(-c2cccc(-c3cccc(-c4ccccc4)c3-n3c4ccccc4c4c(-n5c6ccccc6c6c(-c7ccccc7)cccc65)cccc43)c2)cc1]

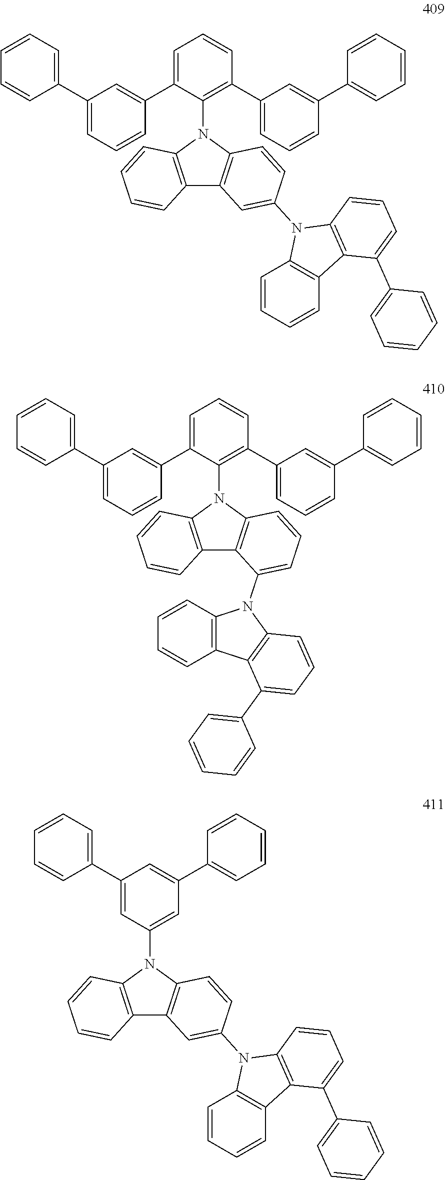

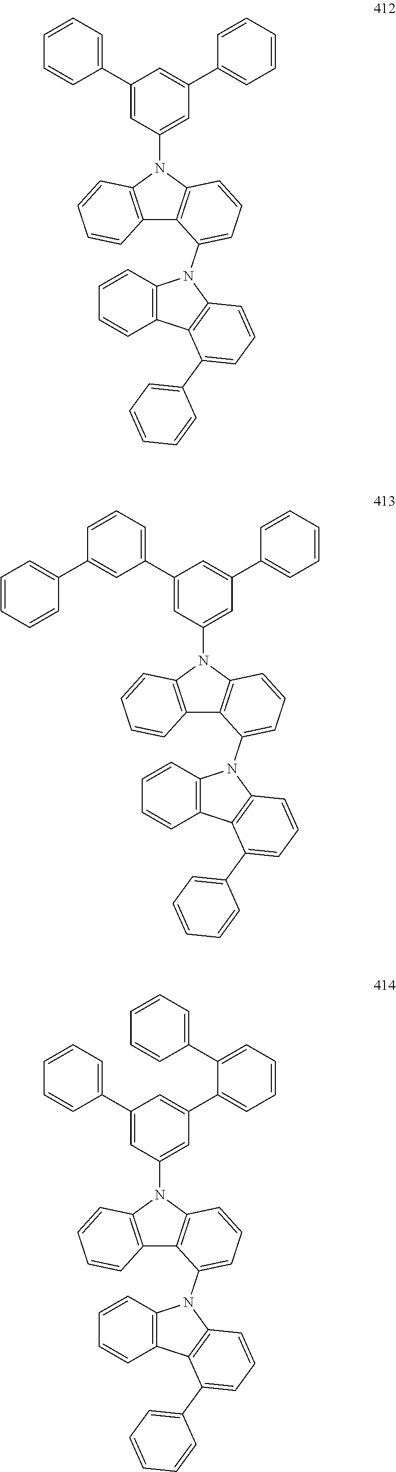 [SMILES: c1ccc(-c2cc(-c3ccccc3)cc(-n3c4ccccc4c4c(-n5c6ccccc6c6c(-c7ccccc7)cccc65)cccc43)c2)cc1 ; c1ccc(-c2cccc(-c3cc(-c4ccccc4)cc(-n4c5ccccc5c5c(-n6c7ccccc7c7c(-c8ccccc8)cccc76)cccc54)c3)c2)cc1 ; c1ccc(-c2cc(-c3ccccc3-c3ccccc3)cc(-n3c4ccccc4c4c(-n5c6ccccc6c6c(-c7ccccc7)cccc65)cccc43)c2)cc1]

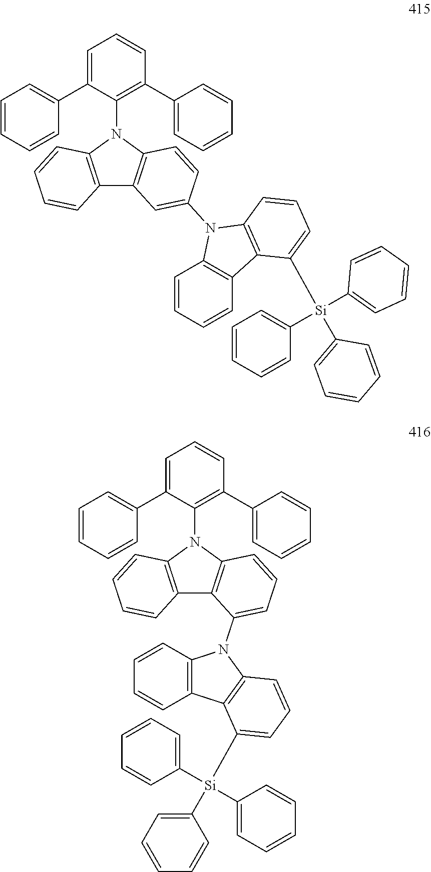 [SMILES: c1ccc(-c2cccc(-c3ccccc3)c2-n2c3ccccc3c3cc(-n4c5ccccc5c5c([Si](c6ccccc6)(c6ccccc6)c6ccccc6)cccc54)ccc32)cc1 ; c1ccc(-c2cccc(-c3ccccc3)c2-n2c3ccccc3c3c(-n4c5ccccc5c5c([Si](c6ccccc6)(c6ccccc6)c6ccccc6)cccc54)cccc32)cc1]

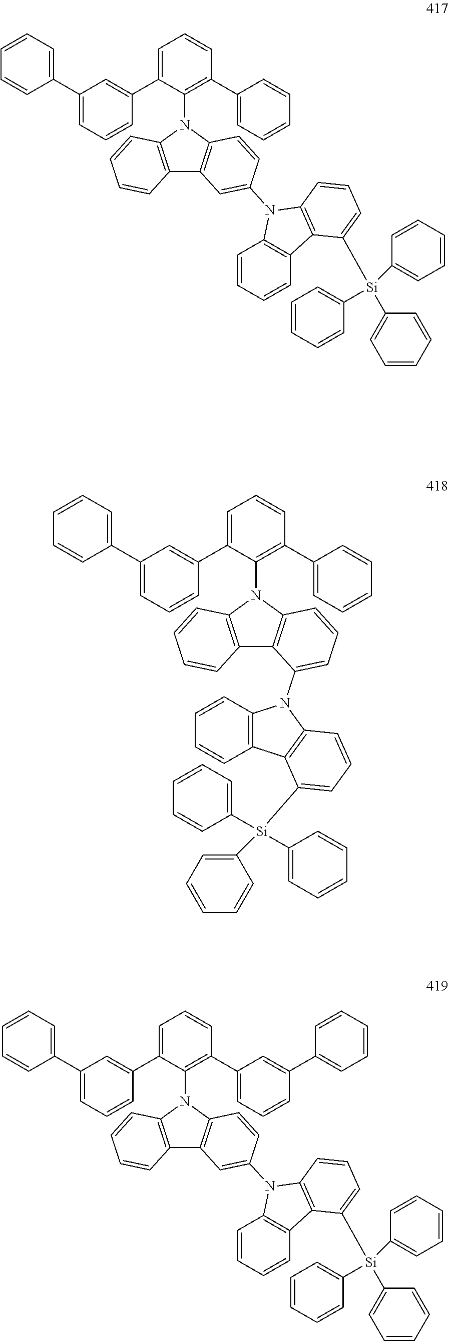 [SMILES: c1ccc(-c2cccc(-c3cccc(-c4ccccc4)c3-n3c4ccccc4c4cc(-n5c6ccccc6c6c([Si](c7ccccc7)(c7ccccc7)c7ccccc7)cccc65)ccc43)c2)cc1 ; c1ccc(-c2cccc(-c3cccc(-c4ccccc4)c3-n3c4ccccc4c4c(-n5c6ccccc6c6c([Si](c7ccccc7)(c7ccccc7)c7ccccc7)cccc65)cccc43)c2)cc1 ; c1ccc(-c2cccc(-c3cccc(-c4cccc(-c5ccccc5)c4)c3-n3c4ccccc4c4cc(-n5c6ccccc6c6c([Si](c7ccccc7)(c7ccccc7)c7ccccc7)cccc65)ccc43)c2)cc1]

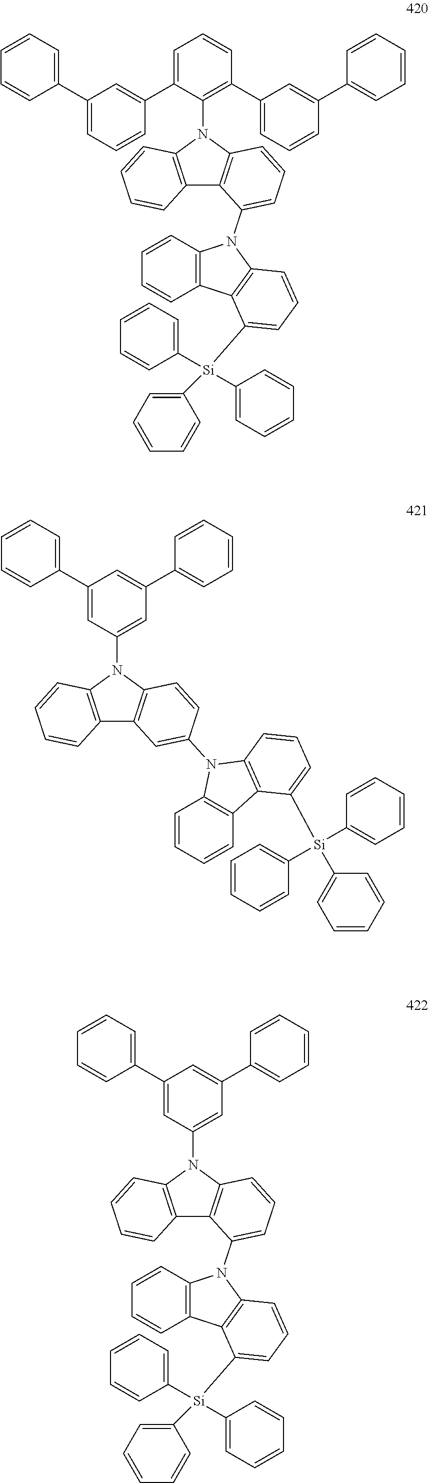 [SMILES: c1ccc(-c2cccc(-c3cccc(-c4cccc(-c5ccccc5)c4)c3-n3c4ccccc4c4c(-n5c6ccccc6c6c([Si](c7ccccc7)(c7ccccc7)c7ccccc7)cccc65)cccc43)c2)cc1 ; c1ccc(-c2cc(-c3ccccc3)cc(-n3c4ccccc4c4cc(-n5c6ccccc6c6c([Si](c7ccccc7)(c7ccccc7)c7ccccc7)cccc65)ccc43)c2)cc1 ; c1ccc(-c2cc(-c3ccccc3)cc(-n3c4ccccc4c4c(-n5c6ccccc6c6c([Si](c7ccccc7)(c7ccccc7)c7ccccc7)cccc65)cccc43)c2)cc1]

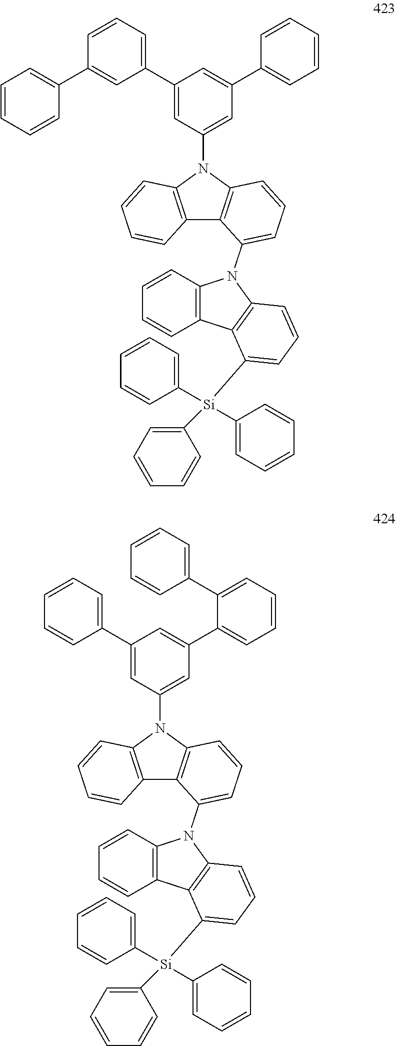

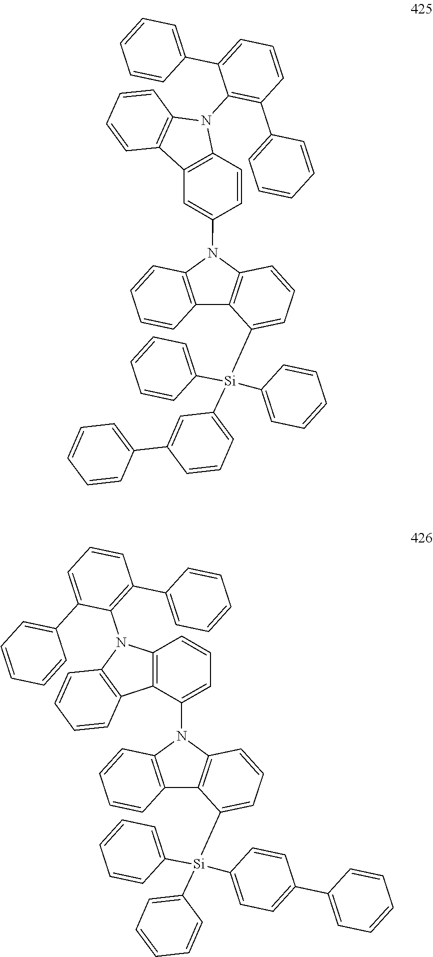 [SMILES: c1ccc(-c2cccc([Si](c3ccccc3)(c3ccccc3)c3cccc4c3c3ccccc3n4-c3ccc4c(c3)c3ccccc3n4-c3c(-c4ccccc4)cccc3-c3ccccc3)c2)cc1 ; c1ccc(-c2ccc([Si](c3ccccc3)(c3ccccc3)c3cccc4c3c3ccccc3n4-c3cccc4c3c3ccccc3n4-c3c(-c4ccccc4)cccc3-c3ccccc3)cc2)cc1]

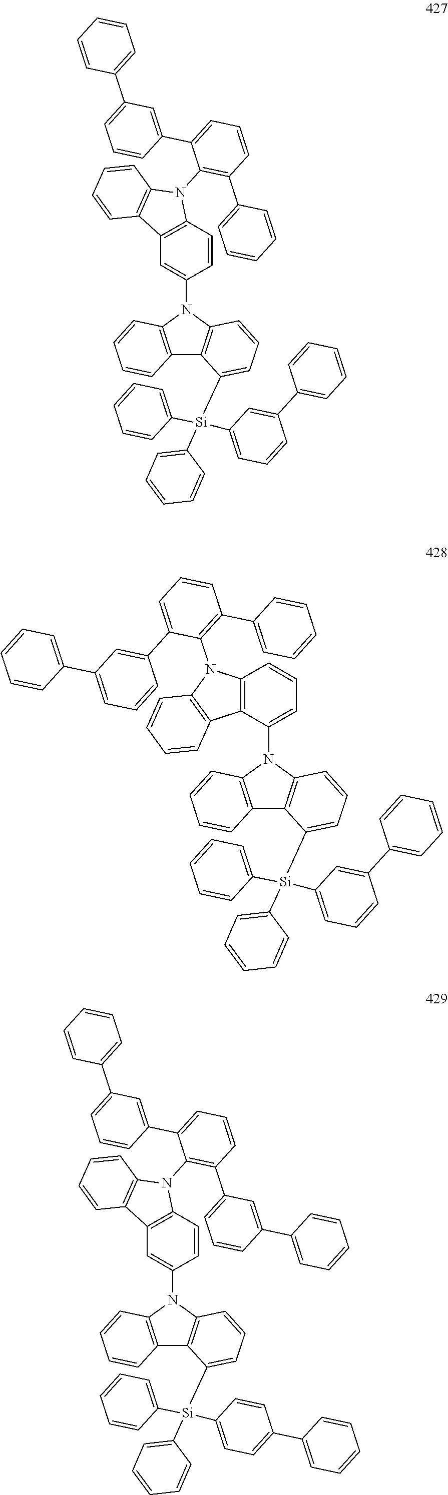 [SMILES: c1ccc(-c2cccc(-c3cccc(-c4ccccc4)c3-n3c4ccccc4c4cc(-n5c6ccccc6c6c([Si](c7ccccc7)(c7ccccc7)c7cccc(-c8ccccc8)c7)cccc65)ccc43)c2)cc1 ; c1ccc(-c2cccc(-c3cccc(-c4ccccc4)c3-n3c4ccccc4c4c(-n5c6ccccc6c6c([Si](c7ccccc7)(c7ccccc7)c7cccc(-c8ccccc8)c7)cccc65)cccc43)c2)cc1 ; c1ccc(-c2ccc([Si](c3ccccc3)(c3ccccc3)c3cccc4c3c3ccccc3n4-c3ccc4c(c3)c3ccccc3n4-c3c(-c4cccc(-c5ccccc5)c4)cccc3-c3cccc(-c4ccccc4)c3)cc2)cc1]

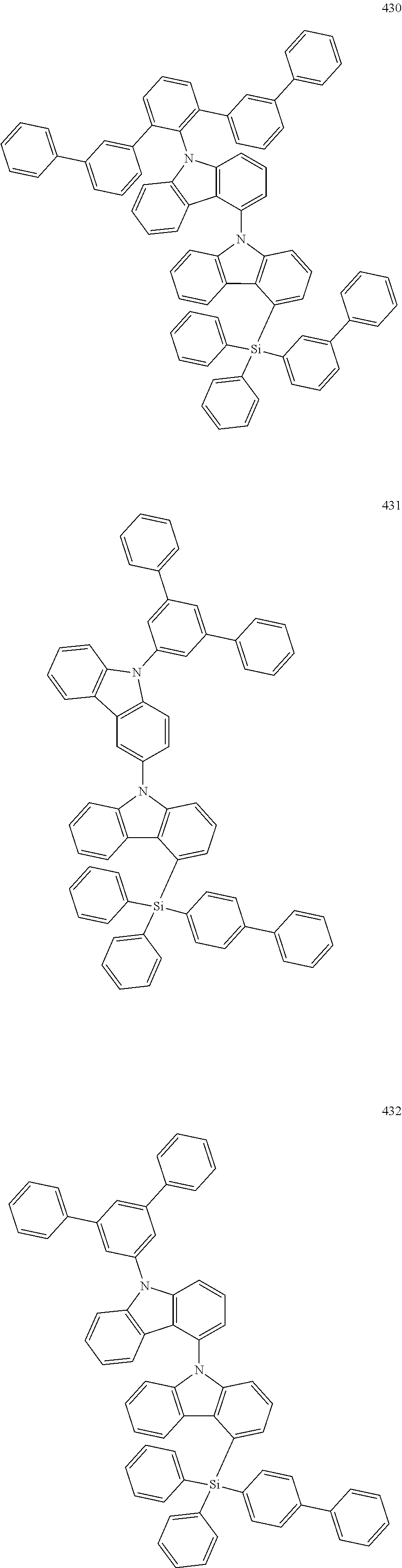 [SMILES: c1ccc(-c2cccc(-c3cccc(-c4cccc(-c5ccccc5)c4)c3-n3c4ccccc4c4c(-n5c6ccccc6c6c([Si](c7ccccc7)(c7ccccc7)c7cccc(-c8ccccc8)c7)cccc65)cccc43)c2)cc1 ; c1ccc(-c2ccc([Si](c3ccccc3)(c3ccccc3)c3cccc4c3c3ccccc3n4-c3ccc4c(c3)c3ccccc3n4-c3cc(-c4ccccc4)cc(-c4ccccc4)c3)cc2)cc1 ; c1ccc(-c2ccc([Si](c3ccccc3)(c3ccccc3)c3cccc4c3c3ccccc3n4-c3cccc4c3c3ccccc3n4-c3cc(-c4ccccc4)cc(-c4ccccc4)c3)cc2)cc1]

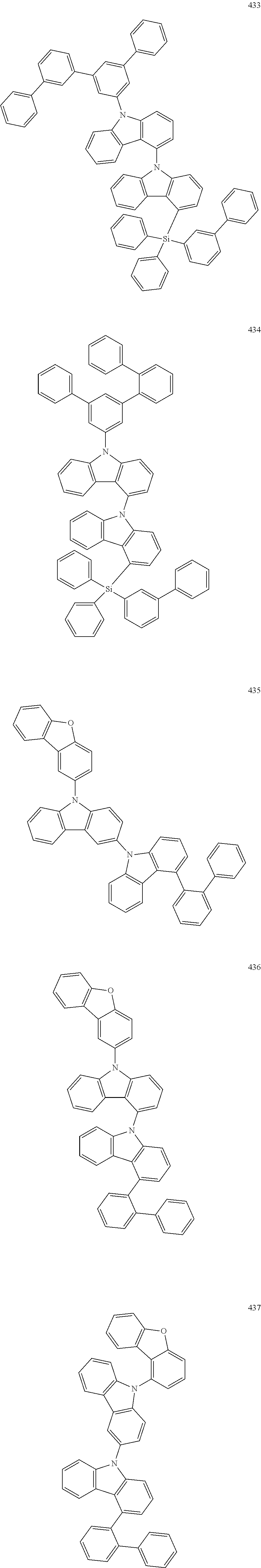

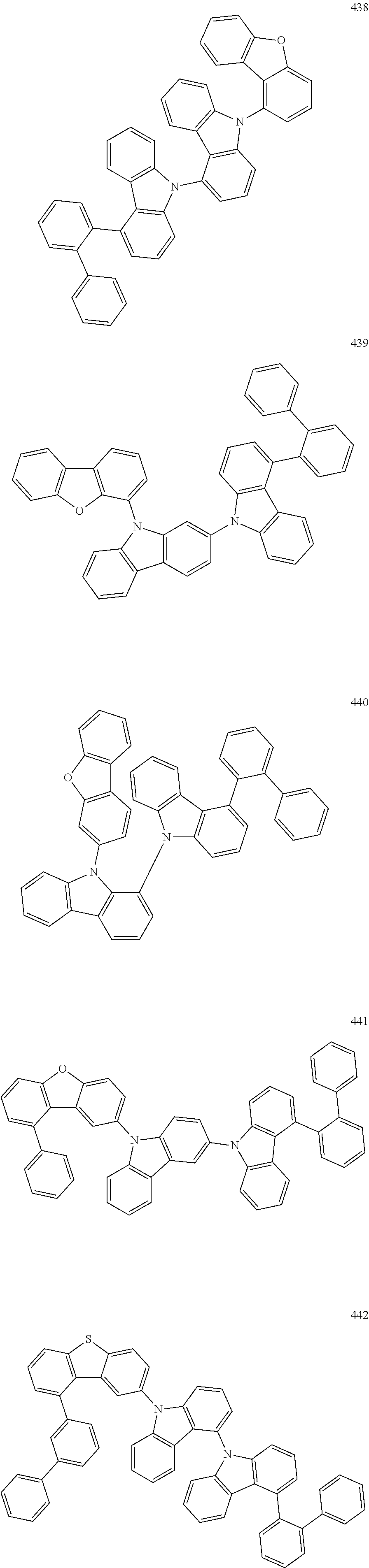

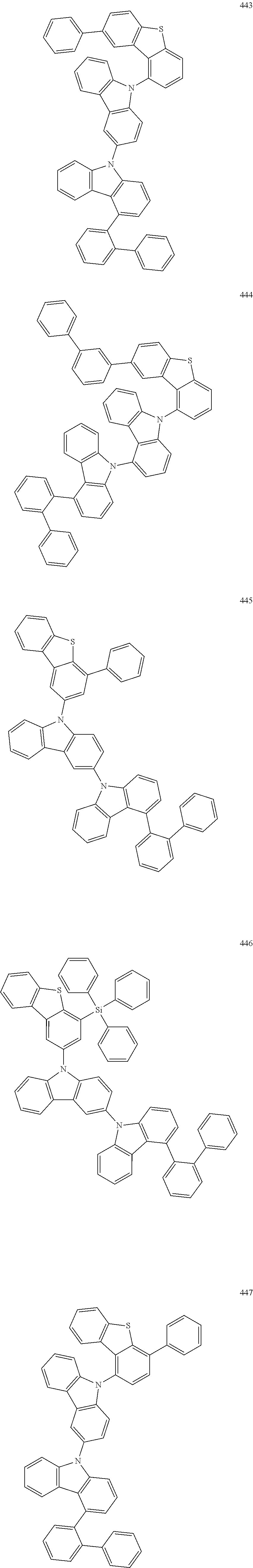

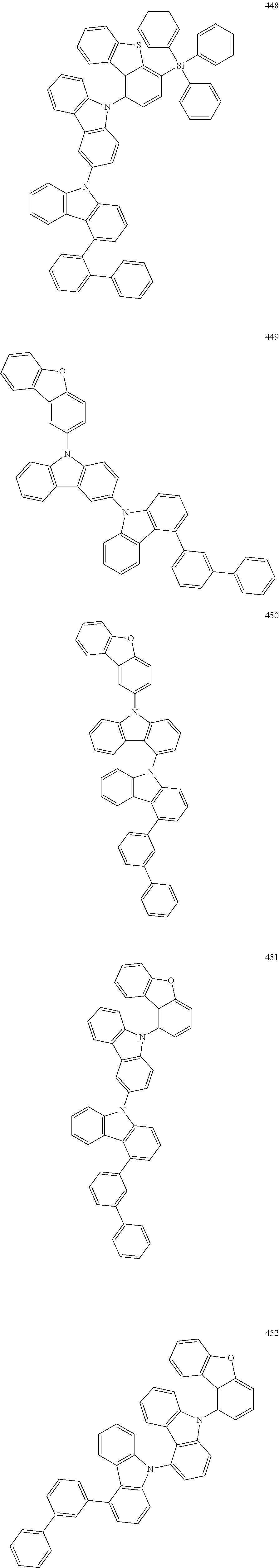 [SMILES: c1ccc(-c2ccccc2-c2cccc3c2c2ccccc2n3-c2ccc3c(c2)c2ccccc2n3-c2ccc([Si](c3ccccc3)(c3ccccc3)c3ccccc3)c3sc4ccccc4c23)cc1 ; c1ccc(-c2cccc(-c3cccc4c3c3ccccc3n4-c3ccc4c(c3)c3ccccc3n4-c3ccc4oc5ccccc5c4c3)c2)cc1 ; c1ccc(-c2cccc(-c3cccc4c3c3ccccc3n4-c3cccc4c3c3ccccc3n4-c3ccc4oc5ccccc5c4c3)c2)cc1 ; c1ccc(-c2cccc(-c3cccc4c3c3ccccc3n4-c3ccc4c(c3)c3ccccc3n4-c3cccc4oc5ccccc5c34)c2)cc1 ; c1ccc(-c2cccc(-c3cccc4c3c3ccccc3n4-c3cccc4c3c3ccccc3n4-c3cccc4oc5ccccc5c34)c2)cc1]

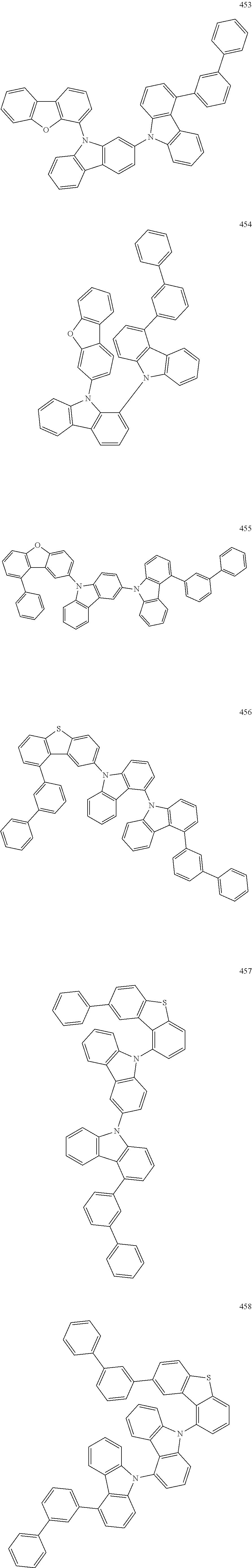

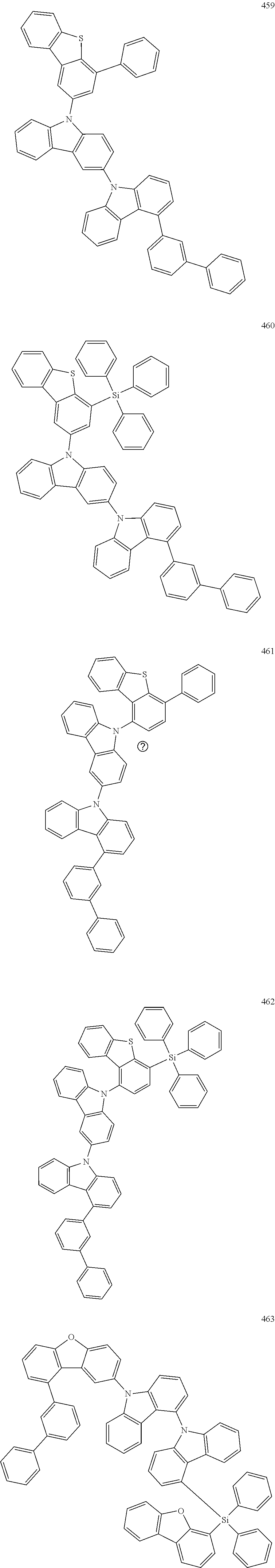

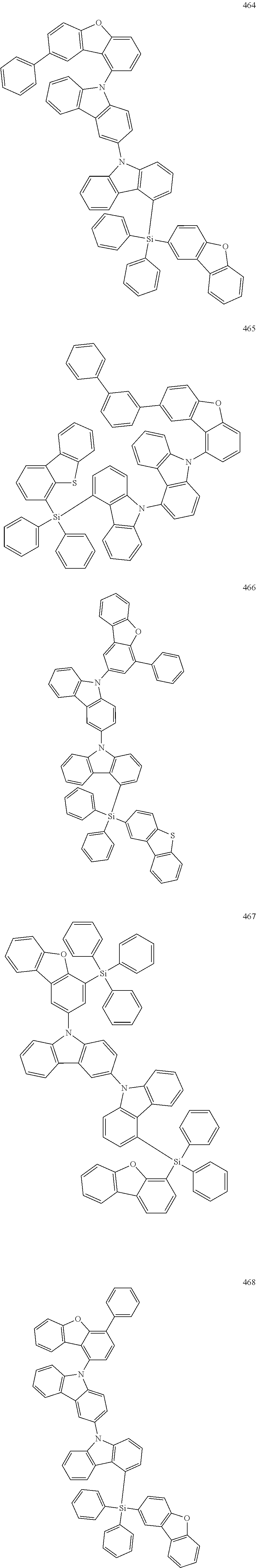

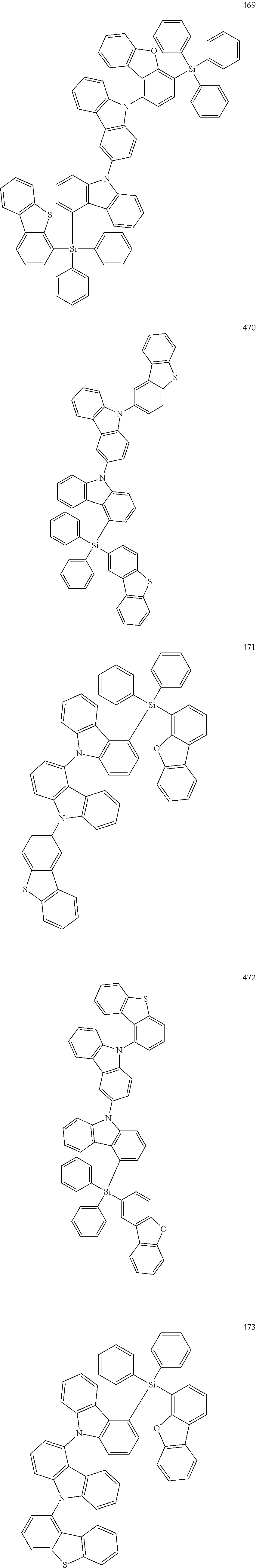

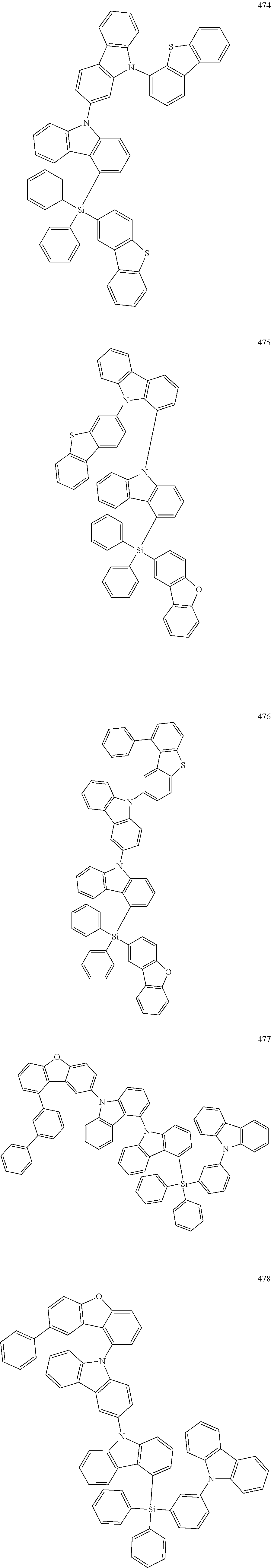

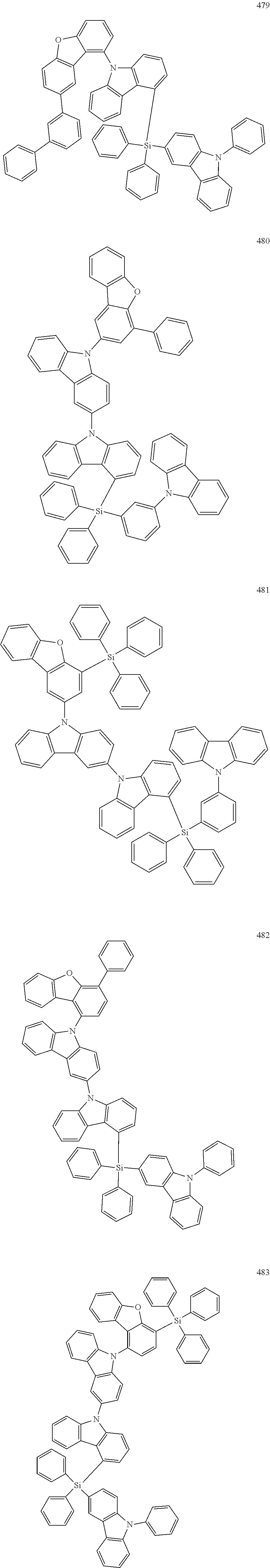

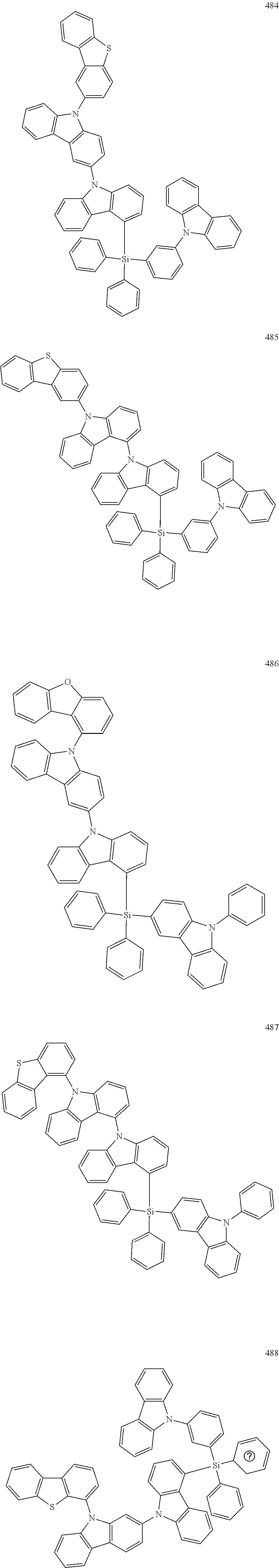

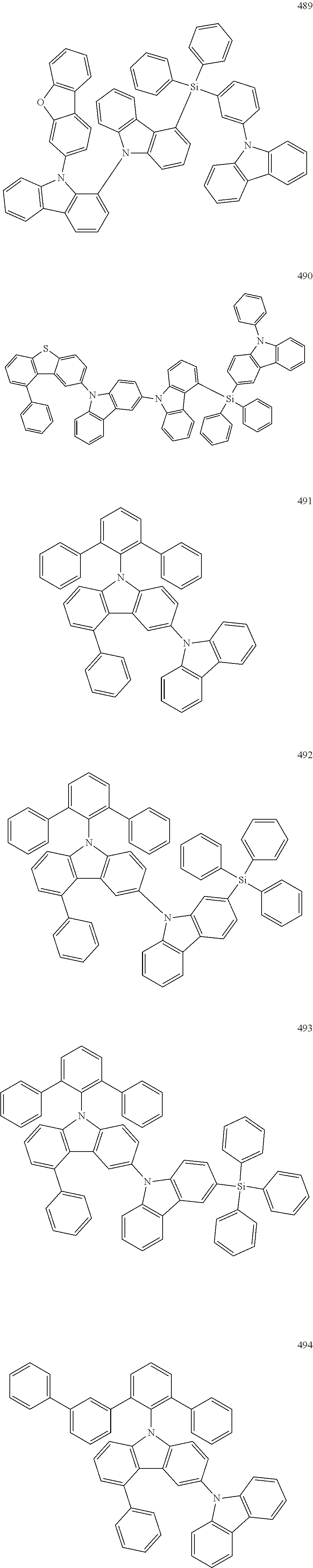

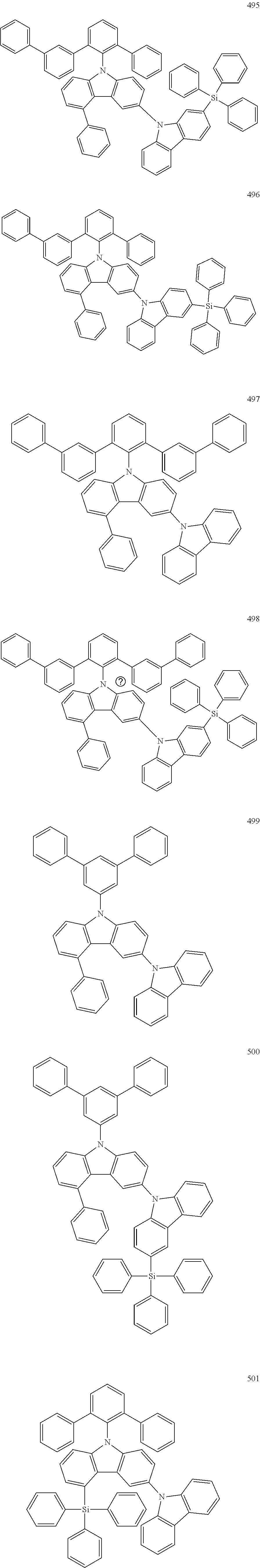

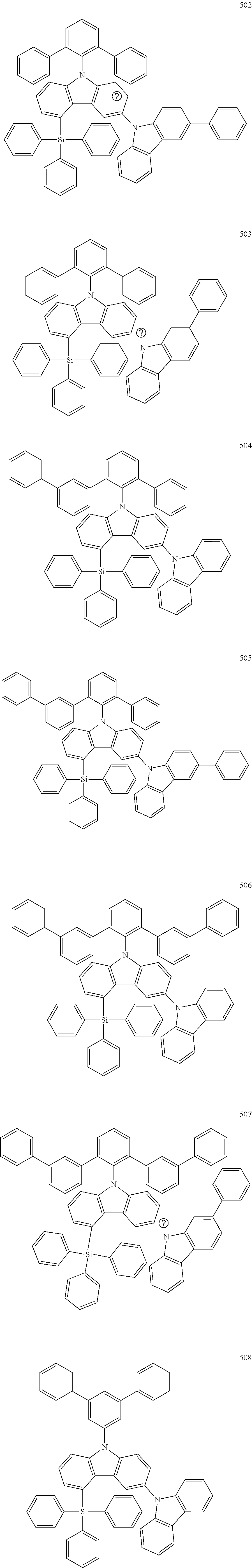

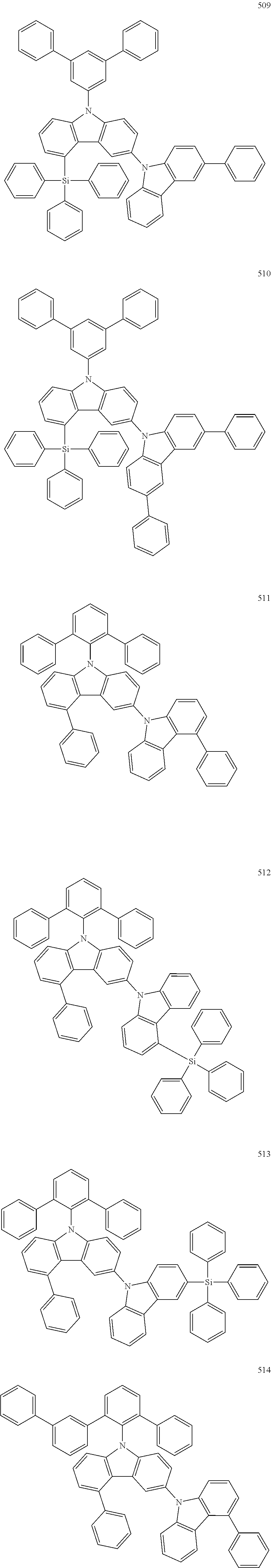

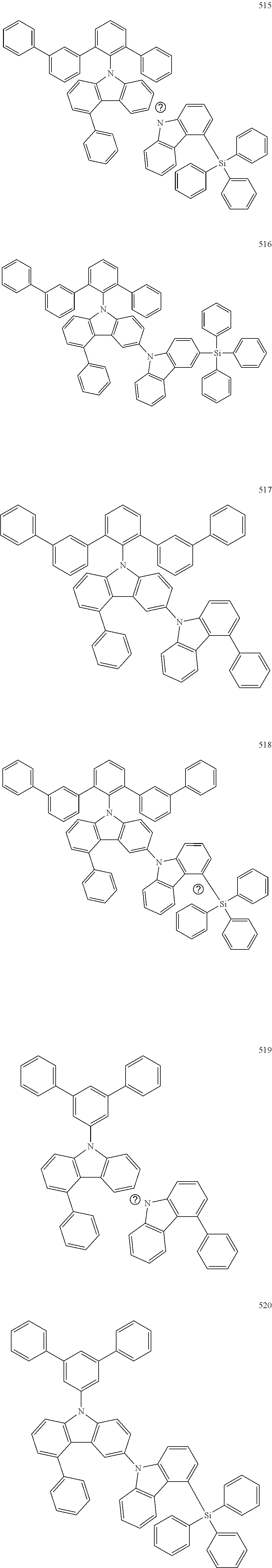

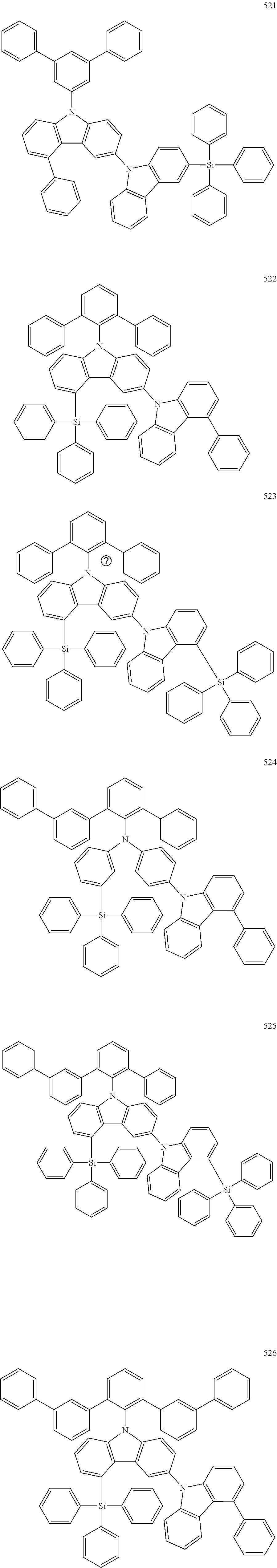

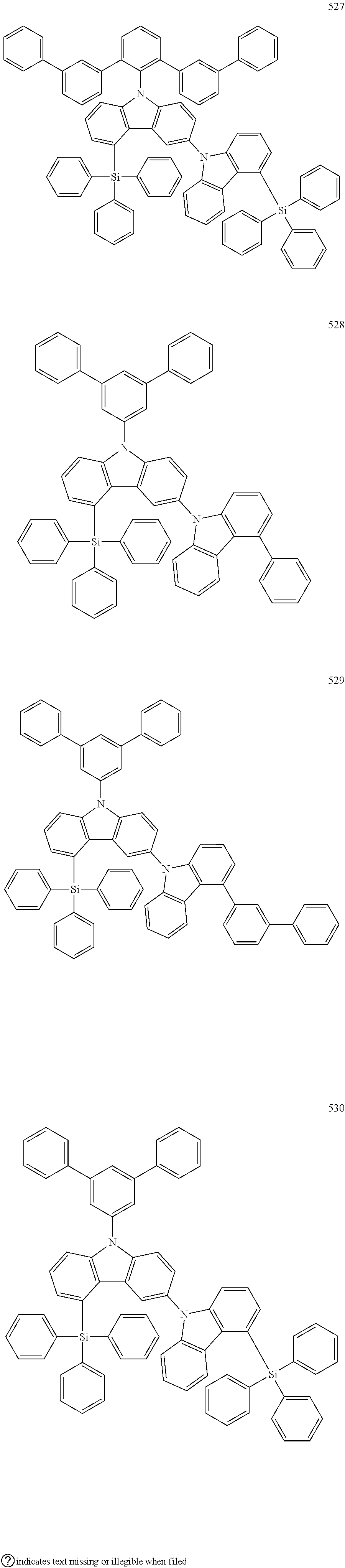

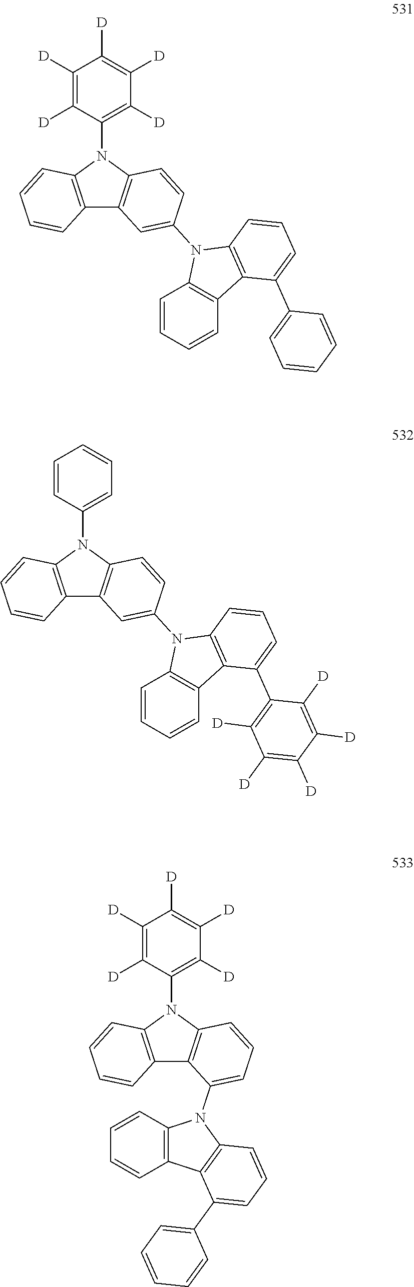 [SMILES: [2H]c1c([2H])c([2H])c(-n2c3ccccc3c3cc(-n4c5ccccc5c5c(-c6ccccc6)cccc54)ccc32)c([2H])c1[2H] ; [2H]c1c([2H])c([2H])c(-c2cccc3c2c2ccccc2n3-c2ccc3c(c2)c2ccccc2n3-c2ccccc2)c([2H])c1[2H] ; [2H]c1c([2H])c([2H])c(-n2c3ccccc3c3c(-n4c5ccccc5c5c(-c6ccccc6)cccc54)cccc32)c([2H])c1[2H]]

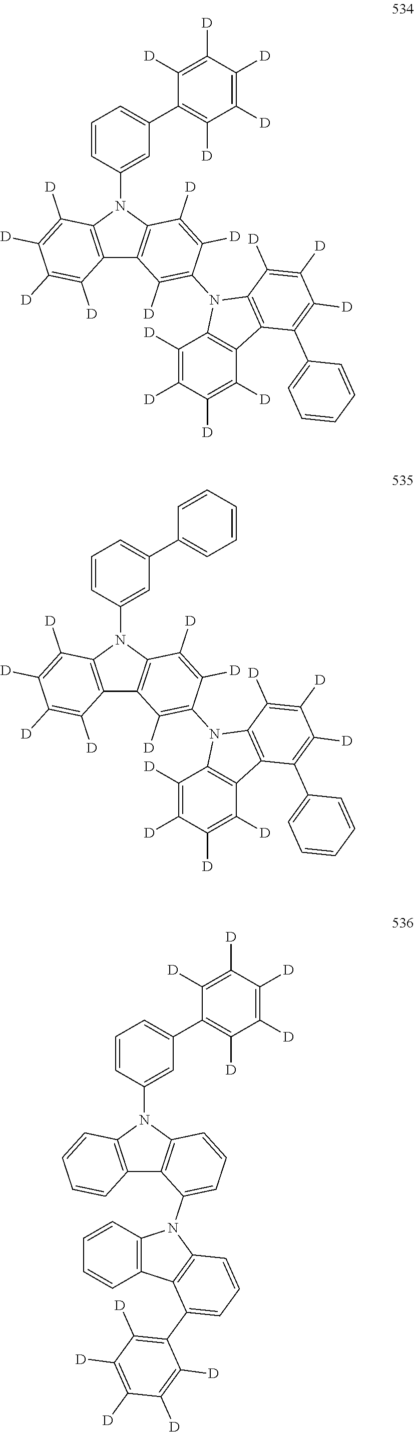 [SMILES: [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c(-c7ccccc7)c([2H])c([2H])c([2H])c65)c([2H])c([2H])c43)c2)c([2H])c1[2H] ; [2H]c1c([2H])c([2H])c2c(c1[2H])c1c([2H])c(-n3c4c([2H])c([2H])c([2H])c([2H])c4c4c(-c5ccccc5)c([2H])c([2H])c([2H])c43)c([2H])c([2H])c1n2-c1cccc(-c2ccccc2)c1 ; [2H]c1c([2H])c([2H])c(-c2cccc(-n3c4ccccc4c4c(-n5c6ccccc6c6c(-c7c([2H])c([2H])c([2H])c([2H])c7[2H])cccc65)cccc43)c2)c([2H])c1[2H]]

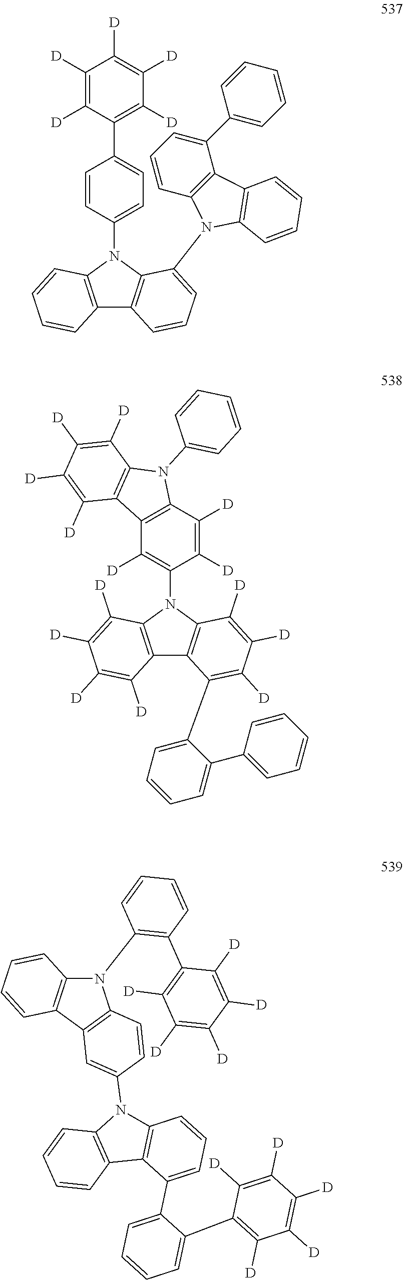 [SMILES: [2H]c1c([2H])c([2H])c(-c2ccc(-n3c4ccccc4c4cccc(-n5c6ccccc6c6c(-c7ccccc7)cccc65)c43)cc2)c([2H])c1[2H] ; [2H]c1c([2H])c([2H])c2c(c1[2H])c1c([2H])c(-n3c4c([2H])c([2H])c([2H])c([2H])c4c4c(-c5ccccc5-c5ccccc5)c([2H])c([2H])c([2H])c43)c([2H])c([2H])c1n2-c1ccccc1 ; [2H]c1c([2H])c([2H])c(-c2ccccc2-c2cccc3c2c2ccccc2n3-c2ccc3c(c2)c2ccccc2n3-c2ccccc2-c2c([2H])c([2H])c([2H])c([2H])c2[2H])c([2H])c1[2H]]

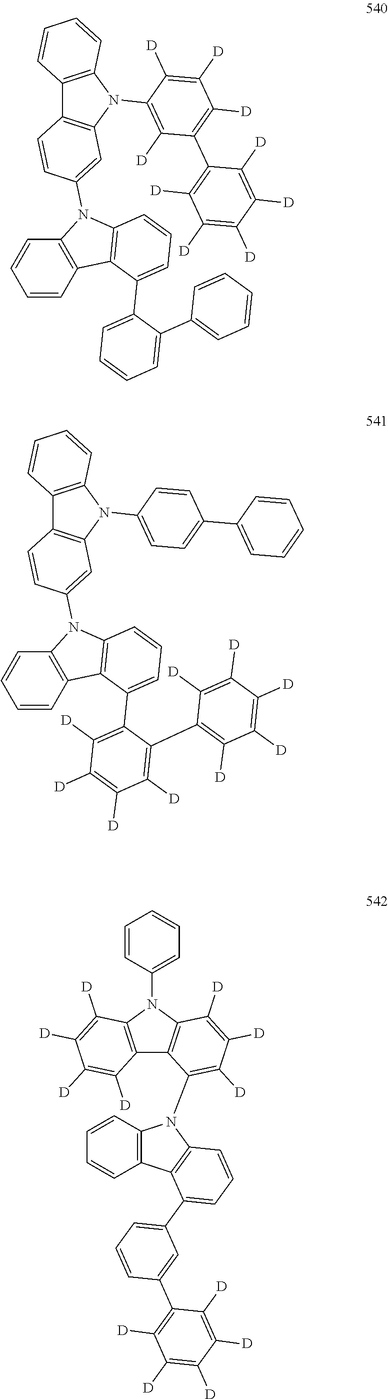 [SMILES: [2H]c1c([2H])c([2H])c(-c2c([2H])c([2H])c([2H])c(-n3c4ccccc4c4ccc(-n5c6ccccc6c6c(-c7ccccc7-c7ccccc7)cccc65)cc43)c2[2H])c([2H])c1[2H] ; [2H]c1c([2H])c([2H])c(-c2c([2H])c([2H])c([2H])c([2H])c2-c2cccc3c2c2ccccc2n3-c2ccc3c4ccccc4n(-c4ccc(-c5ccccc5)cc4)c3c2)c([2H])c1[2H] ; [2H]c1c([2H])c([2H])c(-c2cccc(-c3cccc4c3c3ccccc3n4-c3c([2H])c([2H])c([2H])c4c3c3c([2H])c([2H])c([2H])c([2H])c3n4-c3ccccc3)c2)c([2H])c1[2H]]

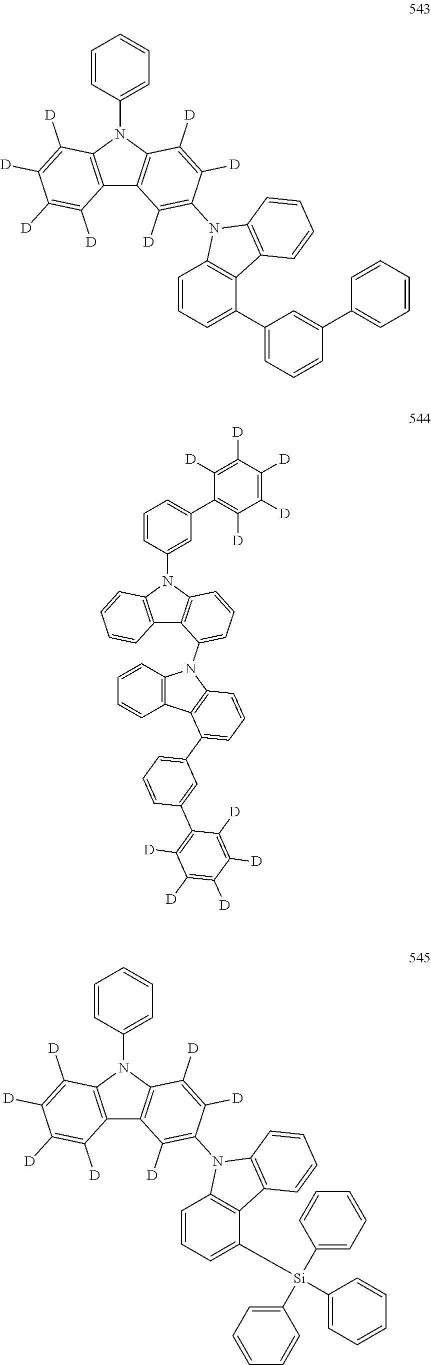 [SMILES: [2H]c1c([2H])c([2H])c2c(c1[2H])c1c([2H])c(-n3c4ccccc4c4c(-c5cccc(-c6ccccc6)c5)cccc43)c([2H])c([2H])c1n2-c1ccccc1 ; [2H]c1c([2H])c([2H])c(-c2cccc(-c3cccc4c3c3ccccc3n4-c3cccc4c3c3ccccc3n4-c3cccc(-c4c([2H])c([2H])c([2H])c([2H])c4[2H])c3)c2)c([2H])c1[2H] ; [2H]c1c([2H])c([2H])c2c(c1[2H])c1c([2H])c(-n3c4ccccc4c4c([Si](c5ccccc5)(c5ccccc5)c5ccccc5)cccc43)c([2H])c([2H])c1n2-c1ccccc1]

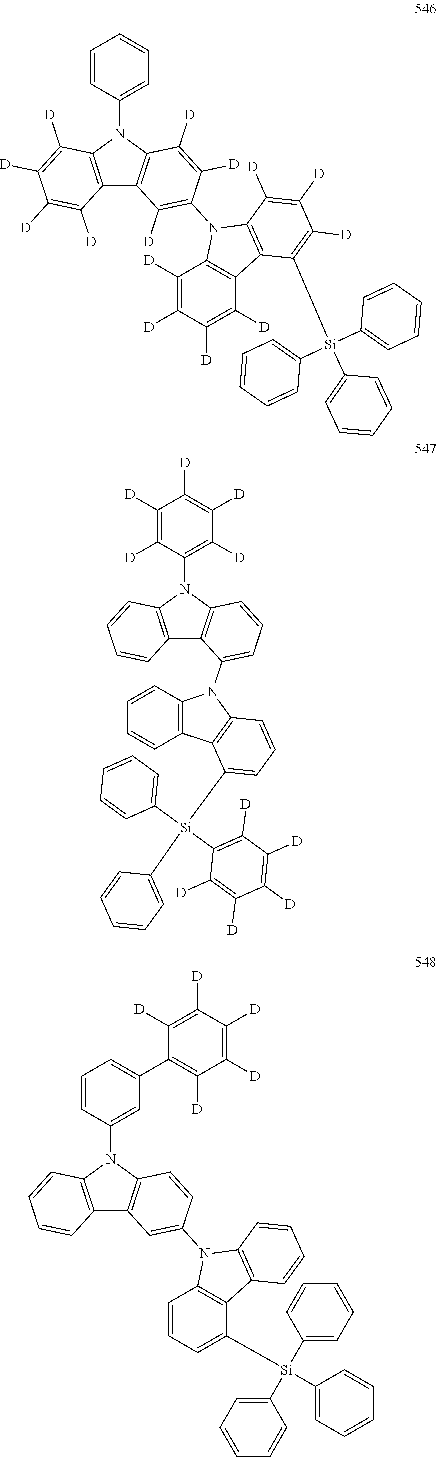 [SMILES: [2H]c1c([2H])c([2H])c2c(c1[2H])c1c([2H])c(-n3c4c([2H])c([2H])c([2H])c([2H])c4c4c([Si](c5ccccc5)(c5ccccc5)c5ccccc5)c([2H])c([2H])c([2H])c43)c([2H])c([2H])c1n2-c1ccccc1 ; [2H]c1c([2H])c([2H])c(-n2c3ccccc3c3c(-n4c5ccccc5c5c([Si](c6ccccc6)(c6ccccc6)c6c([2H])c([2H])c([2H])c([2H])c6[2H])cccc54)cccc32)c([2H])c1[2H] ; [2H]c1c([2H])c([2H])c(-c2cccc(-n3c4ccccc4c4cc(-n5c6ccccc6c6c([Si](c7ccccc7)(c7ccccc7)c7ccccc7)cccc65)ccc43)c2)c([2H])c1[2H]]

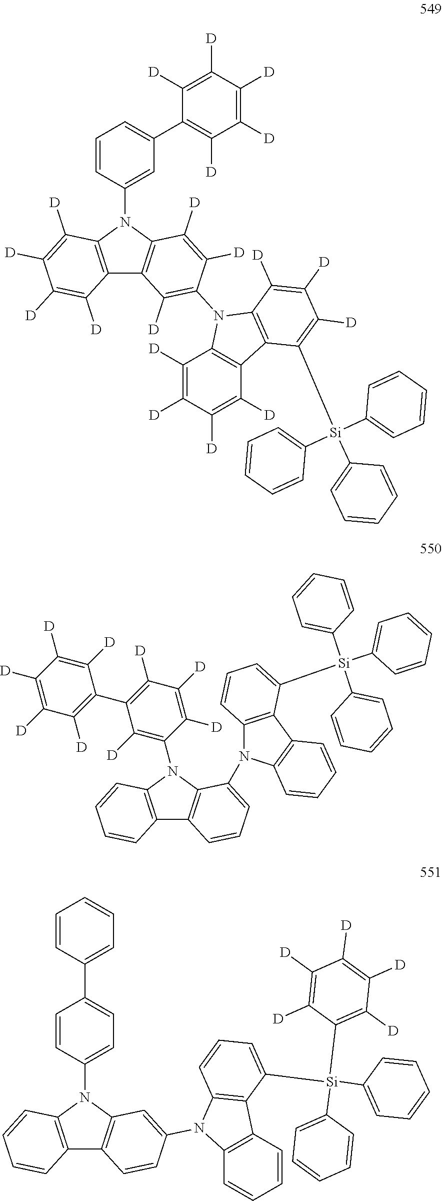 [SMILES: [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c([Si](c7ccccc7)(c7ccccc7)c7ccccc7)c([2H])c([2H])c([2H])c65)c([2H])c([2H])c43)c2)c([2H])c1[2H] ; [2H]c1c([2H])c([2H])c(-c2c([2H])c([2H])c([2H])c(-n3c4ccccc4c4cccc(-n5c6ccccc6c6c([Si](c7ccccc7)(c7ccccc7)c7ccccc7)cccc65)c43)c2[2H])c([2H])c1[2H] ; [2H]c1c([2H])c([2H])c([Si](c2ccccc2)(c2ccccc2)c2cccc3c2c2ccccc2n3-c2ccc3c4ccccc4n(-c4ccc(-c5ccccc5)cc4)c3c2)c([2H])c1[2H]]

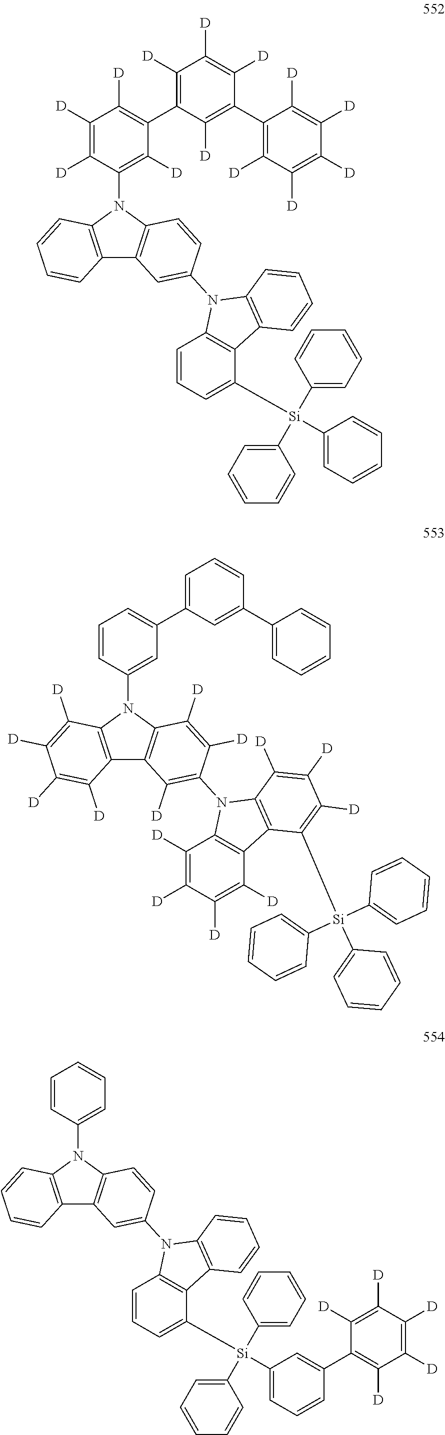 [SMILES: [2H]c1c([2H])c([2H])c(-c2c([2H])c([2H])c([2H])c(-c3c([2H])c([2H])c([2H])c(-n4c5ccccc5c5cc(-n6c7ccccc7c7c([Si](c8ccccc8)(c8ccccc8)c8ccccc8)cccc76)ccc54)c3[2H])c2[2H])c([2H])c1[2H] ; [2H]c1c([2H])c([2H])c2c(c1[2H])c1c([2H])c(-n3c4c([2H])c([2H])c([2H])c([2H])c4c4c([Si](c5ccccc5)(c5ccccc5)c5ccccc5)c([2H])c([2H])c([2H])c43)c([2H])c([2H])c1n2-c1cccc(-c2cccc(-c3ccccc3)c2)c1 ; [2H]c1c([2H])c([2H])c(-c2cccc([Si](c3ccccc3)(c3ccccc3)c3cccc4c3c3ccccc3n4-c3ccc4c(c3)c3ccccc3n4-c3ccccc3)c2)c([2H])c1[2H]]

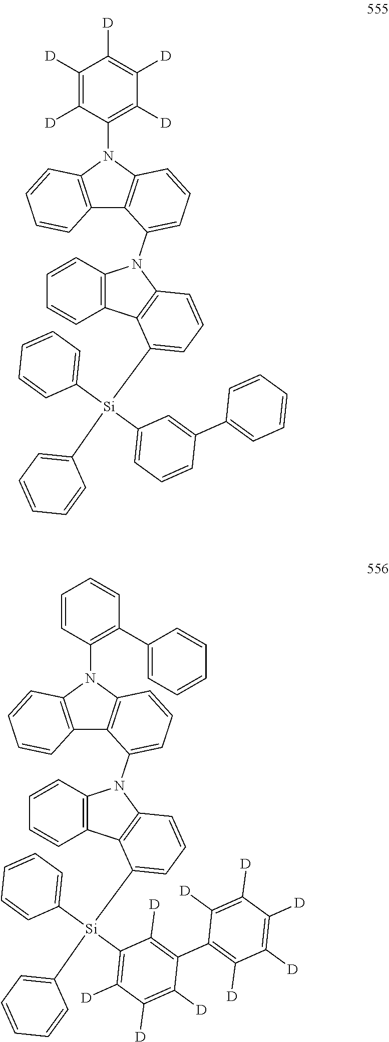 [SMILES: [2H]c1c([2H])c([2H])c(-n2c3ccccc3c3c(-n4c5ccccc5c5c([Si](c6ccccc6)(c6ccccc6)c6cccc(-c7ccccc7)c6)cccc54)cccc32)c([2H])c1[2H] ; [2H]c1c([2H])c([2H])c(-c2c([2H])c([2H])c([2H])c([Si](c3ccccc3)(c3ccccc3)c3cccc4c3c3ccccc3n4-c3cccc4c3c3ccccc3n4-c3ccccc3-c3ccccc3)c2[2H])c([2H])c1[2H]]

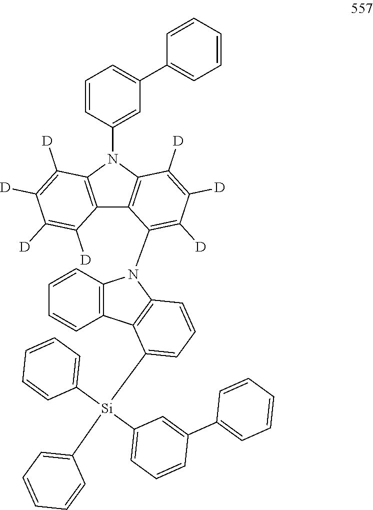 [SMILES: [2H]c1c([2H])c([2H])c2c(c1[2H])c1c(-n3c4ccccc4c4c([Si](c5ccccc5)(c5ccccc5)c5cccc(-c6ccccc6)c5)cccc43)c([2H])c([2H])c([2H])c1n2-c1cccc(-c2ccccc2)c1]

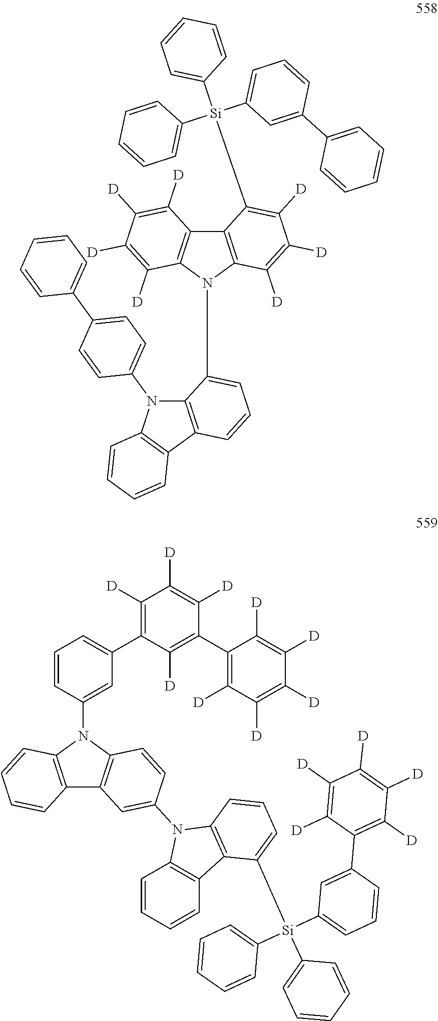 [SMILES: [2H]c1c([2H])c([2H])c2c(c1[2H])c1c([Si](c3ccccc3)(c3ccccc3)c3cccc(-c4ccccc4)c3)c([2H])c([2H])c([2H])c1n2-c1cccc2c3ccccc3n(-c3ccc(-c4ccccc4)cc3)c12 ; [2H]c1c([2H])c([2H])c(-c2cccc([Si](c3ccccc3)(c3ccccc3)c3cccc4c3c3ccccc3n4-c3ccc4c(c3)c3ccccc3n4-c3cccc(-c4c([2H])c([2H])c([2H])c(-c5c([2H])c([2H])c([2H])c([2H])c5[2H])c4[2H])c3)c2)c([2H])c1[2H]]

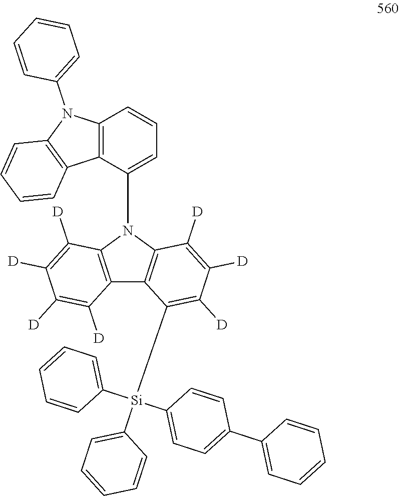 [SMILES: [2H]c1c([2H])c([2H])c2c(c1[2H])c1c([Si](c3ccccc3)(c3ccccc3)c3ccc(-c4ccccc4)cc3)c([2H])c([2H])c([2H])c1n2-c1cccc2c1c1ccccc1n2-c1ccccc1]

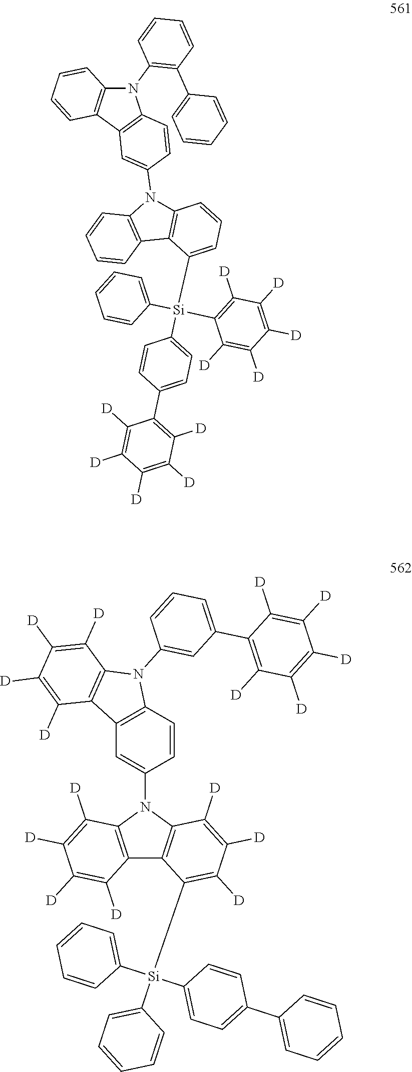 [SMILES: [2H]c1c([2H])c([2H])c(-c2ccc([Si](c3ccccc3)(c3c([2H])c([2H])c([2H])c([2H])c3[2H])c3cccc4c3c3ccccc3n4-c3ccc4c(c3)c3ccccc3n4-c3ccccc3-c3ccccc3)cc2)c([2H])c1[2H] ; [2H]c1c([2H])c([2H])c(-c2cccc(-n3c4ccc(-n5c6c([2H])c([2H])c([2H])c([2H])c6c6c([Si](c7ccccc7)(c7ccccc7)c7ccc(-c8ccccc8)cc7)c([2H])c([2H])c([2H])c65)cc4c4c([2H])c([2H])c([2H])c([2H])c43)c2)c([2H])c1[2H]]

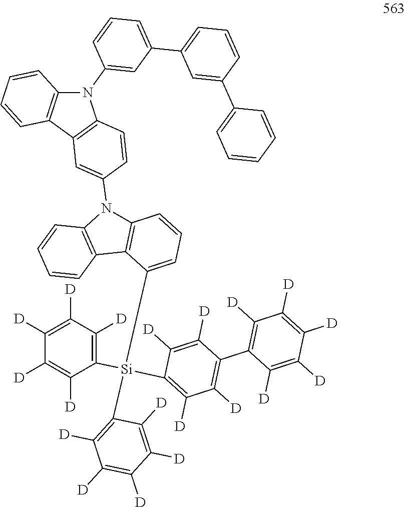 [SMILES: [2H]c1c([2H])c([2H])c(-c2c([2H])c([2H])c([Si](c3c([2H])c([2H])c([2H])c([2H])c3[2H])(c3c([2H])c([2H])c([2H])c([2H])c3[2H])c3cccc4c3c3ccccc3n4-c3ccc4c(c3)c3ccccc3n4-c3cccc(-c4cccc(-c5ccccc5)c4)c3)c([2H])c2[2H])c([2H])c1[2H]]

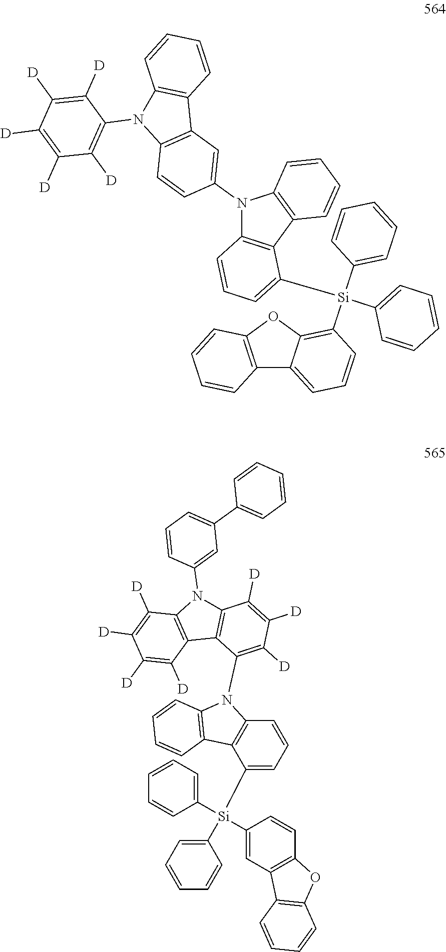 [SMILES: [2H]c1c([2H])c([2H])c(-n2c3ccccc3c3cc(-n4c5ccccc5c5c([Si](c6ccccc6)(c6ccccc6)c6cccc7c6oc6ccccc67)cccc54)ccc32)c([2H])c1[2H] ; [2H]c1c([2H])c([2H])c2c(c1[2H])c1c(-n3c4ccccc4c4c([Si](c5ccccc5)(c5ccccc5)c5ccc6oc7ccccc7c6c5)cccc43)c([2H])c([2H])c([2H])c1n2-c1cccc(-c2ccccc2)c1]

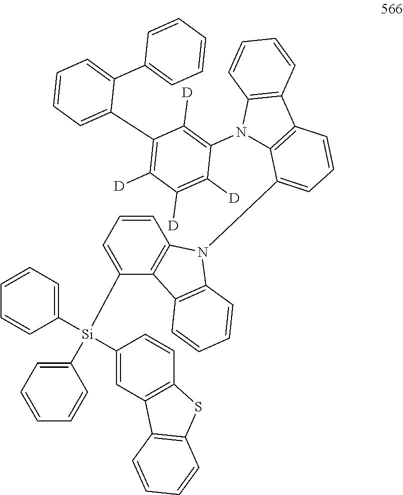 [SMILES: [2H]c1c([2H])c(-c2ccccc2-c2ccccc2)c([2H])c(-n2c3ccccc3c3cccc(-n4c5ccccc5c5c([Si](c6ccccc6)(c6ccccc6)c6ccc7sc8ccccc8c7c6)cccc54)c32)c1[2H]]

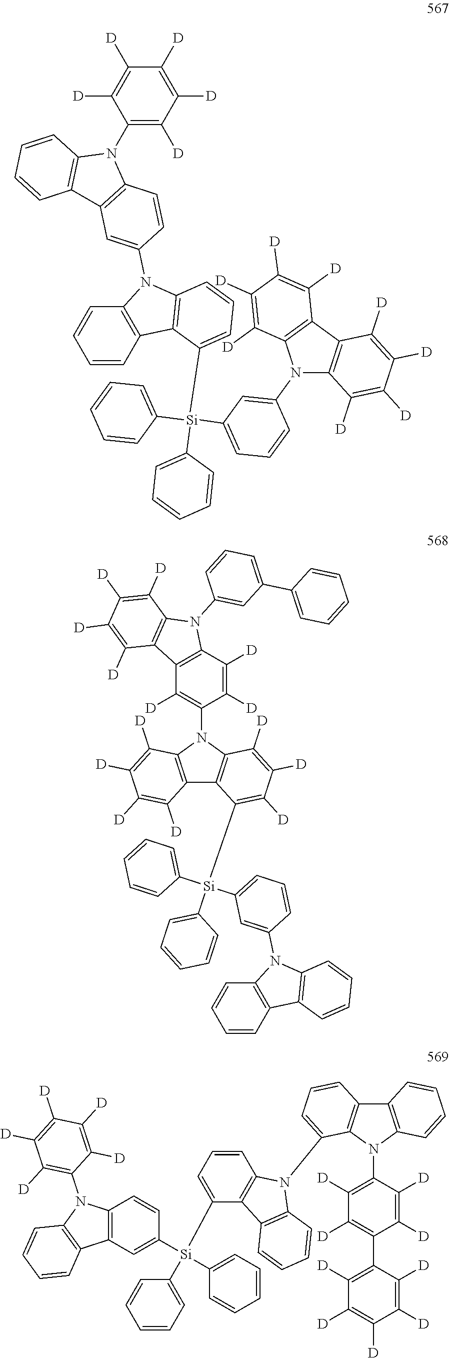

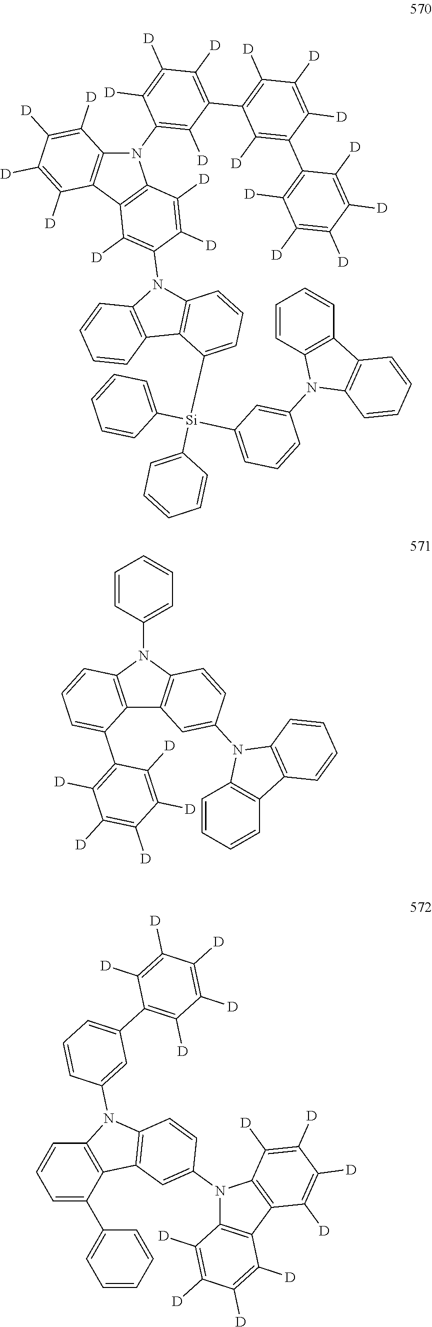

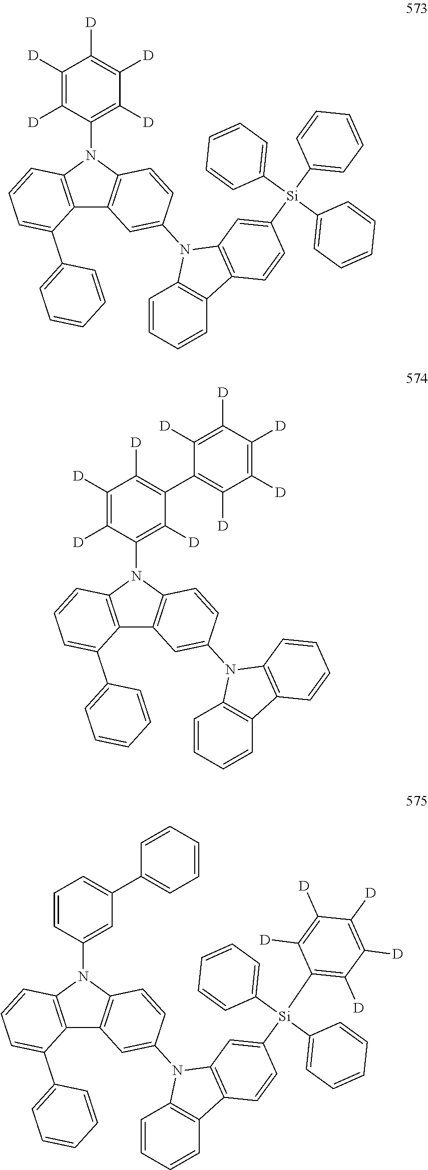 [SMILES: [2H]c1c([2H])c([2H])c(-n2c3ccc(-n4c5ccccc5c5ccc([Si](c6ccccc6)(c6ccccc6)c6ccccc6)cc54)cc3c3c(-c4ccccc4)cccc32)c([2H])c1[2H] ; [2H]c1c([2H])c([2H])c(-c2c([2H])c([2H])c([2H])c(-n3c4ccc(-n5c6ccccc6c6ccccc65)cc4c4c(-c5ccccc5)cccc43)c2[2H])c([2H])c1[2H] ; [2H]c1c([2H])c([2H])c([Si](c2ccccc2)(c2ccccc2)c2ccc3c4ccccc4n(-c4ccc5c(c4)c4c(-c6ccccc6)cccc4n5-c4cccc(-c5ccccc5)c4)c3c2)c([2H])c1[2H]]

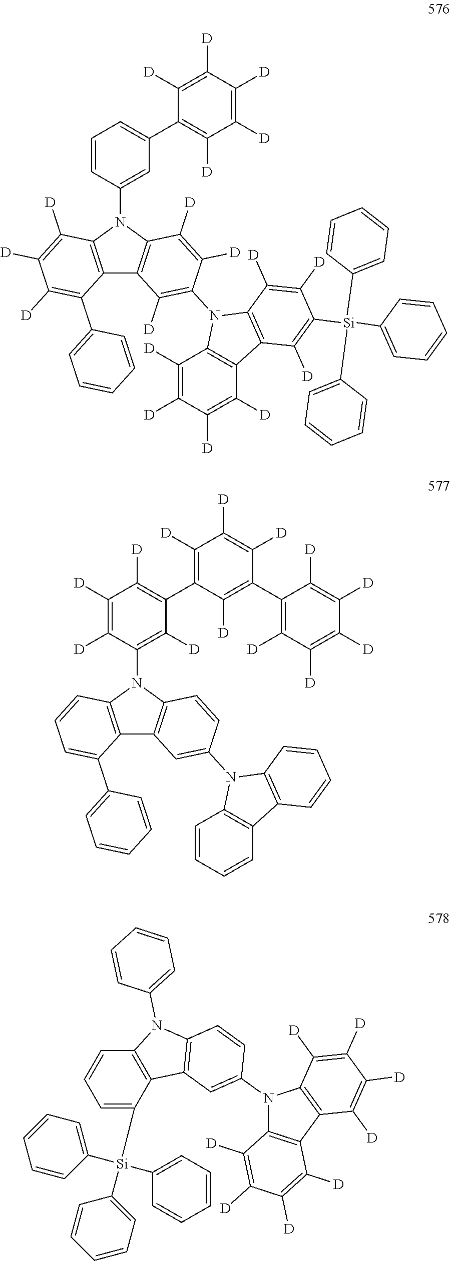

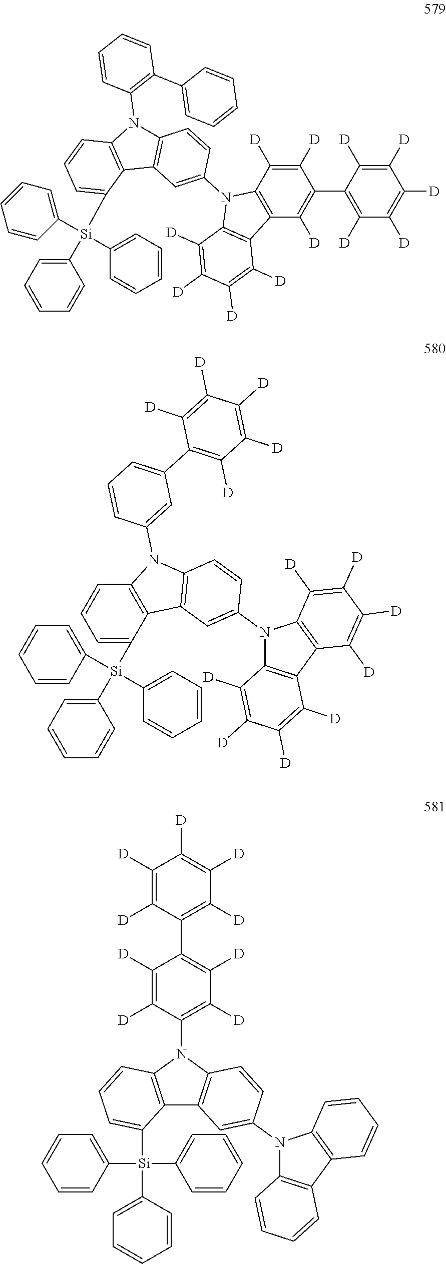 [SMILES: [2H]c1c([2H])c([2H])c(-c2c([2H])c([2H])c3c(c2[2H])c2c([2H])c([2H])c([2H])c([2H])c2n3-c2ccc3c(c2)c2c([Si](c4ccccc4)(c4ccccc4)c4ccccc4)cccc2n3-c2ccccc2-c2ccccc2)c([2H])c1[2H] ; [2H]c1c([2H])c([2H])c(-c2cccc(-n3c4ccc(-n5c6c([2H])c([2H])c([2H])c([2H])c6c6c([2H])c([2H])c([2H])c([2H])c65)cc4c4c([Si](c5ccccc5)(c5ccccc5)c5ccccc5)cccc43)c2)c([2H])c1[2H] ; [2H]c1c([2H])c([2H])c(-c2c([2H])c([2H])c(-n3c4ccc(-n5c6ccccc6c6ccccc65)cc4c4c([Si](c5ccccc5)(c5ccccc5)c5ccccc5)cccc43)c([2H])c2[2H])c([2H])c1[2H]]

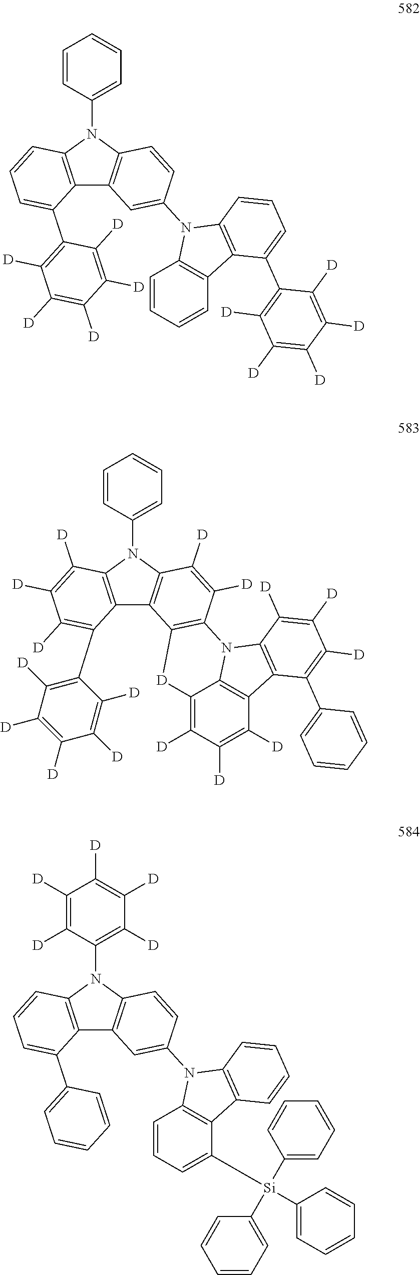

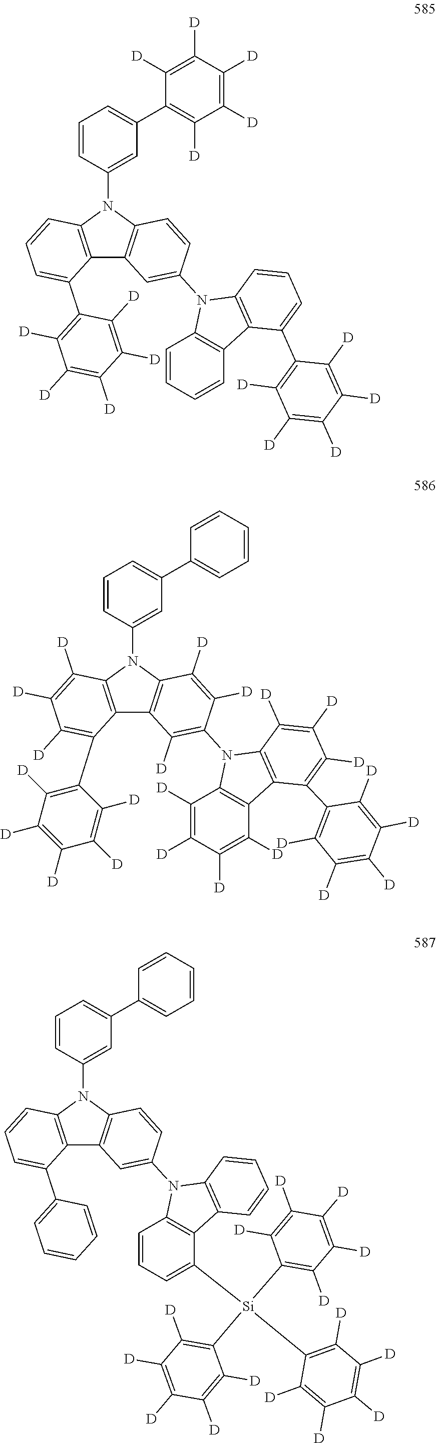

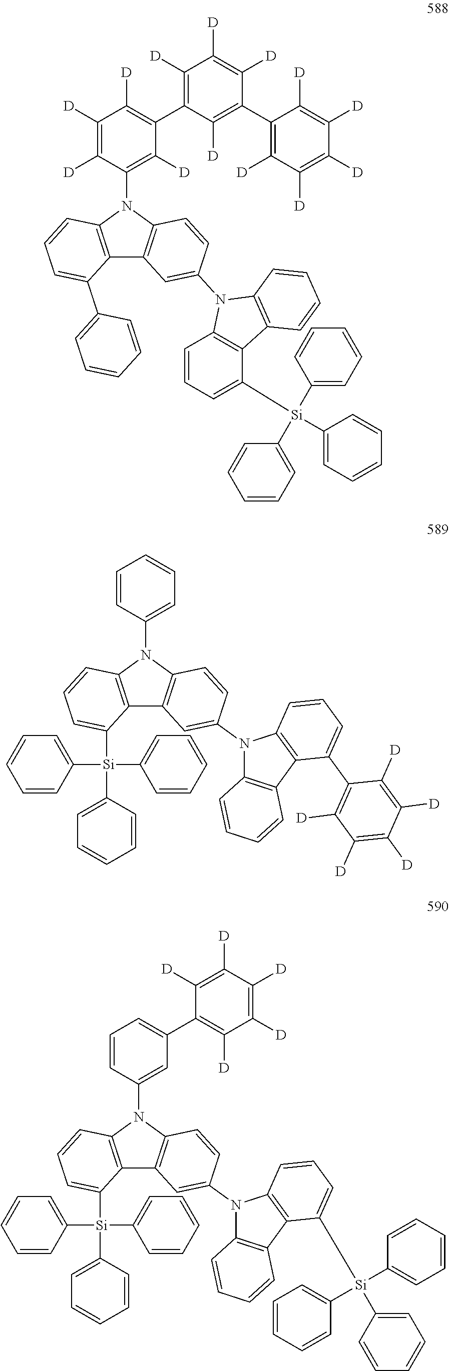 [SMILES: [2H]c1c([2H])c([2H])c(-c2c([2H])c([2H])c([2H])c(-c3c([2H])c([2H])c([2H])c(-n4c5ccc(-n6c7ccccc7c7c([Si](c8ccccc8)(c8ccccc8)c8ccccc8)cccc76)cc5c5c(-c6ccccc6)cccc54)c3[2H])c2[2H])c([2H])c1[2H] ; [2H]c1c([2H])c([2H])c(-c2cccc3c2c2ccccc2n3-c2ccc3c(c2)c2c([Si](c4ccccc4)(c4ccccc4)c4ccccc4)cccc2n3-c2ccccc2)c([2H])c1[2H] ; [2H]c1c([2H])c([2H])c(-c2cccc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)c2)c([2H])c1[2H]]

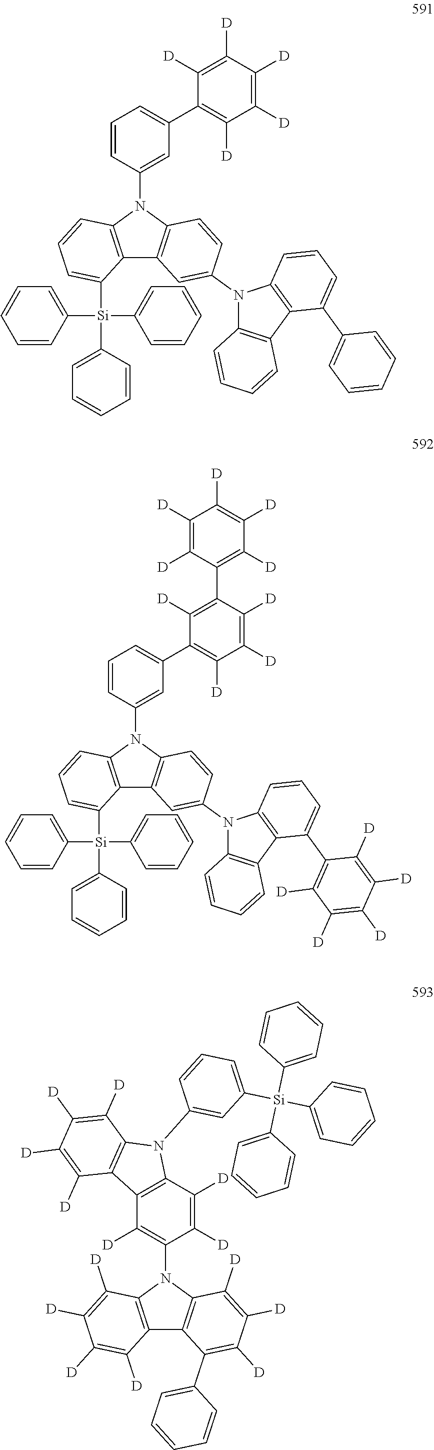

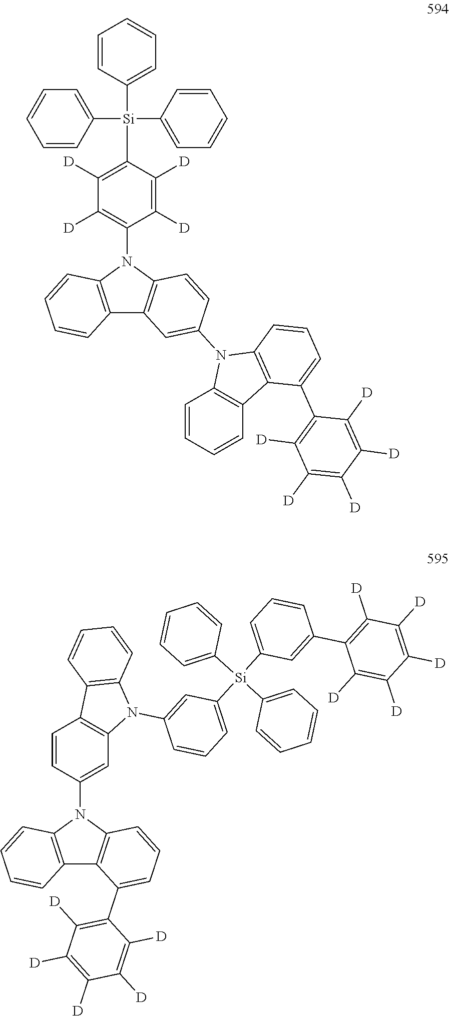 [SMILES: [2H]c1c([2H])c([2H])c(-c2cccc3c2c2ccccc2n3-c2ccc3c(c2)c2ccccc2n3-c2c([2H])c([2H])c([Si](c3ccccc3)(c3ccccc3)c3ccccc3)c([2H])c2[2H])c([2H])c1[2H] ; [2H]c1c([2H])c([2H])c(-c2cccc([Si](c3ccccc3)(c3ccccc3)c3cccc(-n4c5ccccc5c5ccc(-n6c7ccccc7c7c(-c8c([2H])c([2H])c([2H])c([2H])c8[2H])cccc76)cc54)c3)c2)c([2H])c1[2H]]

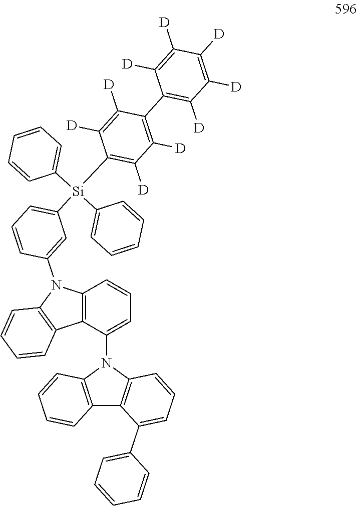 [SMILES: [2H]c1c([2H])c([2H])c(-c2c([2H])c([2H])c([Si](c3ccccc3)(c3ccccc3)c3cccc(-n4c5ccccc5c5c(-n6c7ccccc7c7c(-c8ccccc8)cccc76)cccc54)c3)c([2H])c2[2H])c([2H])c1[2H]]

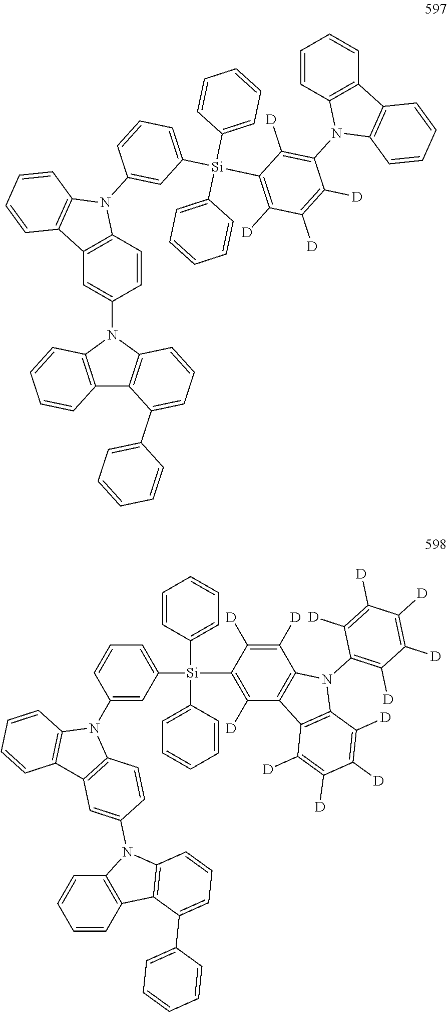 [SMILES: [2H]c1c([2H])c(-n2c3ccccc3c3ccccc32)c([2H])c([Si](c2ccccc2)(c2ccccc2)c2cccc(-n3c4ccccc4c4cc(-n5c6ccccc6c6c(-c7ccccc7)cccc65)ccc43)c2)c1[2H] ; [2H]c1c([2H])c([2H])c(-n2c3c([2H])c([2H])c([2H])c([2H])c3c3c([2H])c([Si](c4ccccc4)(c4ccccc4)c4cccc(-n5c6ccccc6c6cc(-n7c8ccccc8c8c(-c9ccccc9)cccc87)ccc65)c4)c([2H])c([2H])c32)c([2H])c1[2H]]

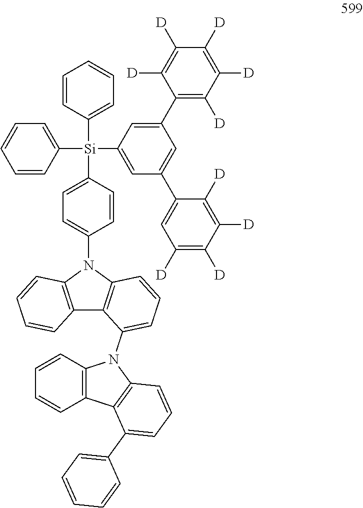 [SMILES: [2H]c1cc(-c2cc(-c3c([2H])c([2H])c([2H])c([2H])c3[2H])cc([Si](c3ccccc3)(c3ccccc3)c3ccc(-n4c5ccccc5c5c(-n6c7ccccc7c7c(-c8ccccc8)cccc76)cccc54)cc3)c2)c([2H])c([2H])c1[2H]]

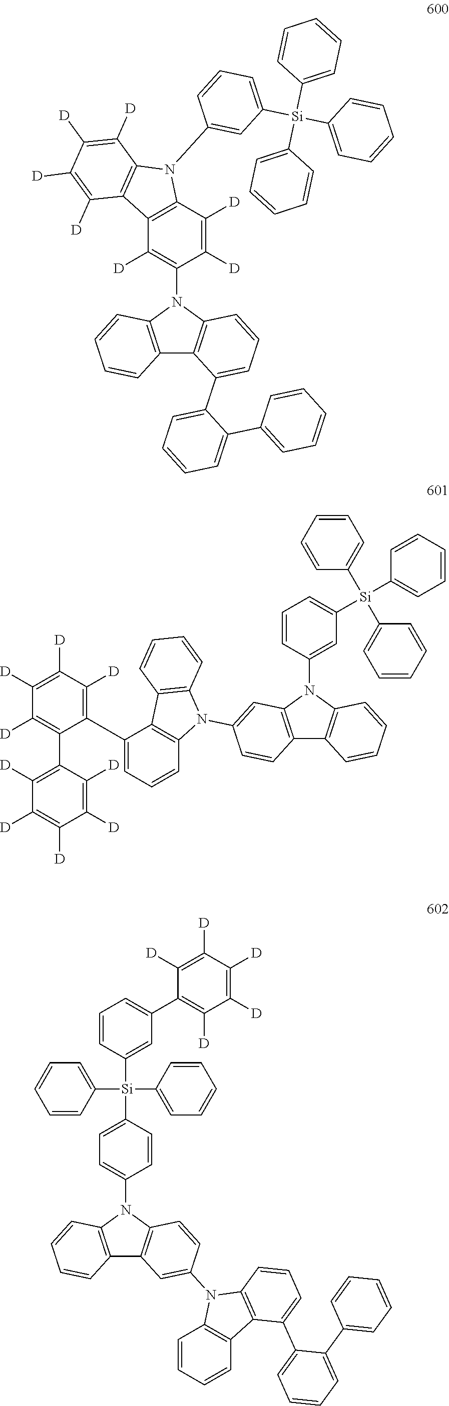 [SMILES: [2H]c1c([2H])c([2H])c2c(c1[2H])c1c([2H])c(-n3c4ccccc4c4c(-c5ccccc5-c5ccccc5)cccc43)c([2H])c([2H])c1n2-c1cccc([Si](c2ccccc2)(c2ccccc2)c2ccccc2)c1 ; [2H]c1c([2H])c([2H])c(-c2c([2H])c([2H])c([2H])c([2H])c2-c2cccc3c2c2ccccc2n3-c2ccc3c4ccccc4n(-c4cccc([Si](c5ccccc5)(c5ccccc5)c5ccccc5)c4)c3c2)c([2H])c1[2H] ; [2H]c1c([2H])c([2H])c(-c2cccc([Si](c3ccccc3)(c3ccccc3)c3ccc(-n4c5ccccc5c5cc(-n6c7ccccc7c7c(-c8ccccc8-c8ccccc8)cccc76)ccc54)cc3)c2)c([2H])c1[2H]]

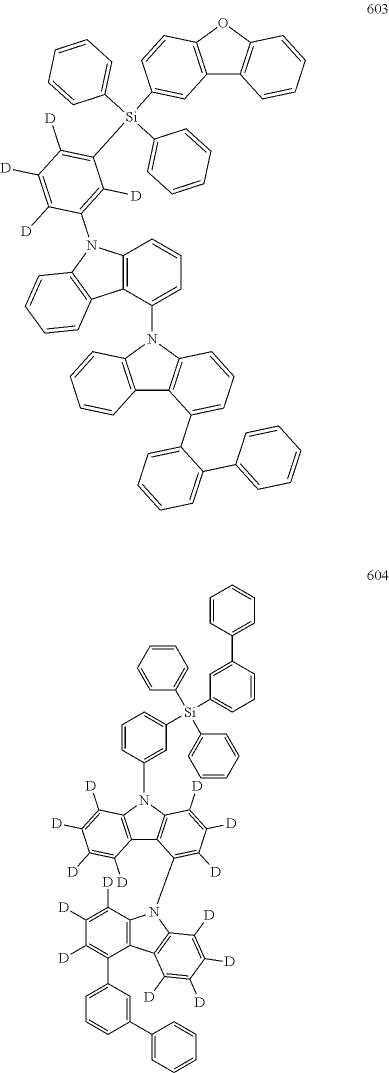 [SMILES: [2H]c1c([2H])c(-n2c3ccccc3c3c(-n4c5ccccc5c5c(-c6ccccc6-c6ccccc6)cccc54)cccc32)c([2H])c([Si](c2ccccc2)(c2ccccc2)c2ccc3oc4ccccc4c3c2)c1[2H] ; [2H]c1c([2H])c([2H])c2c(c1[2H])c1c(-n3c4c([2H])c([2H])c([2H])c([2H])c4c4c(-c5cccc(-c6ccccc6)c5)c([2H])c([2H])c([2H])c43)c([2H])c([2H])c([2H])c1n2-c1cccc([Si](c2ccccc2)(c2ccccc2)c2cccc(-c3ccccc3)c2)c1]

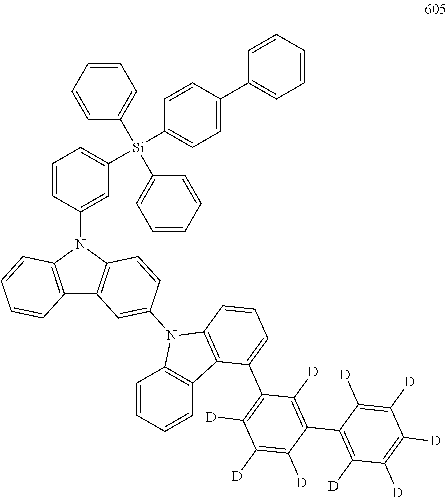 [SMILES: [2H]c1c([2H])c([2H])c(-c2c([2H])c([2H])c([2H])c(-c3cccc4c3c3ccccc3n4-c3ccc4c(c3)c3ccccc3n4-c3cccc([Si](c4ccccc4)(c4ccccc4)c4ccc(-c5ccccc5)cc4)c3)c2[2H])c([2H])c1[2H]]

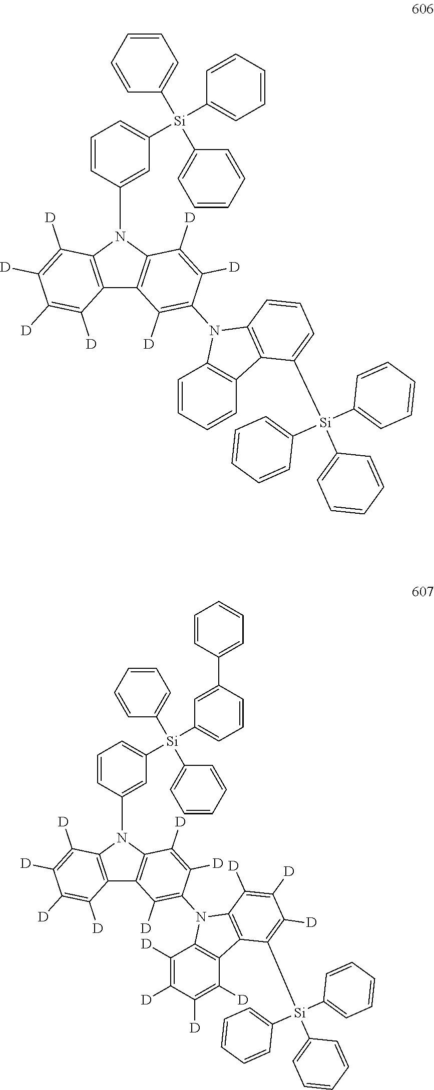 [SMILES: [2H]c1c([2H])c([2H])c2c(c1[2H])c1c([2H])c(-n3c4ccccc4c4c([Si](c5ccccc5)(c5ccccc5)c5ccccc5)cccc43)c([2H])c([2H])c1n2-c1cccc([Si](c2ccccc2)(c2ccccc2)c2ccccc2)c1 ; [2H]c1c([2H])c([2H])c2c(c1[2H])c1c([2H])c(-n3c4c([2H])c([2H])c([2H])c([2H])c4c4c([Si](c5ccccc5)(c5ccccc5)c5ccccc5)c([2H])c([2H])c([2H])c43)c([2H])c([2H])c1n2-c1cccc([Si](c2ccccc2)(c2ccccc2)c2cccc(-c3ccccc3)c2)c1]

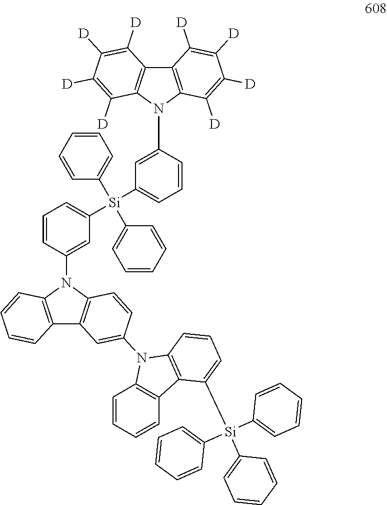 [SMILES: [2H]c1c([2H])c([2H])c2c(c1[2H])c1c([2H])c([2H])c([2H])c([2H])c1n2-c1cccc([Si](c2ccccc2)(c2ccccc2)c2cccc(-n3c4ccccc4c4cc(-n5c6ccccc6c6c([Si](c7ccccc7)(c7ccccc7)c7ccccc7)cccc65)ccc43)c2)c1]

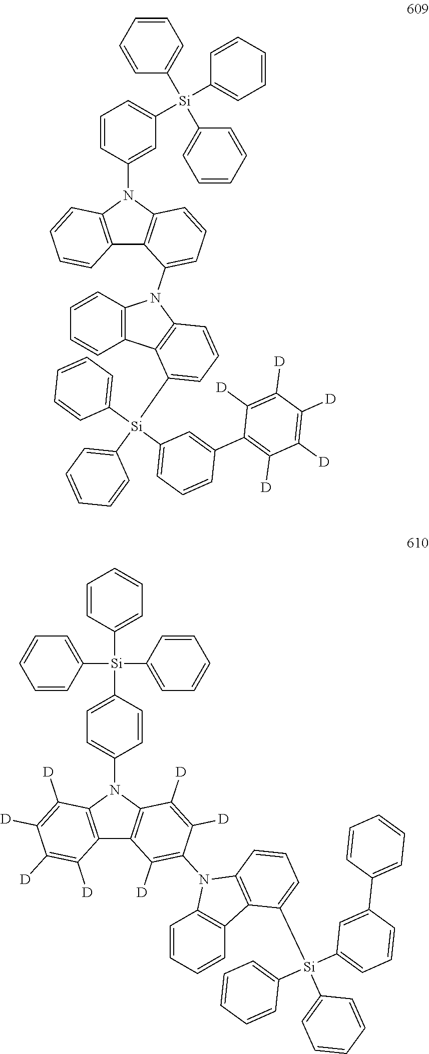 [SMILES: [2H]c1c([2H])c([2H])c(-c2cccc([Si](c3ccccc3)(c3ccccc3)c3cccc4c3c3ccccc3n4-c3cccc4c3c3ccccc3n4-c3cccc([Si](c4ccccc4)(c4ccccc4)c4ccccc4)c3)c2)c([2H])c1[2H] ; [2H]c1c([2H])c([2H])c2c(c1[2H])c1c([2H])c(-n3c4ccccc4c4c([Si](c5ccccc5)(c5ccccc5)c5cccc(-c6ccccc6)c5)cccc43)c([2H])c([2H])c1n2-c1ccc([Si](c2ccccc2)(c2ccccc2)c2ccccc2)cc1]

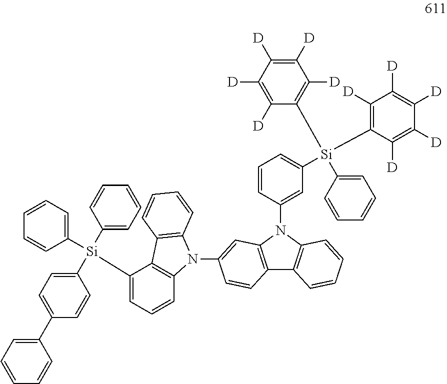 [SMILES: [2H]c1c([2H])c([2H])c([Si](c2ccccc2)(c2cccc(-n3c4ccccc4c4ccc(-n5c6ccccc6c6c([Si](c7ccccc7)(c7ccccc7)c7ccc(-c8ccccc8)cc7)cccc65)cc43)c2)c2c([2H])c([2H])c([2H])c([2H])c2[2H])c([2H])c1[2H]]

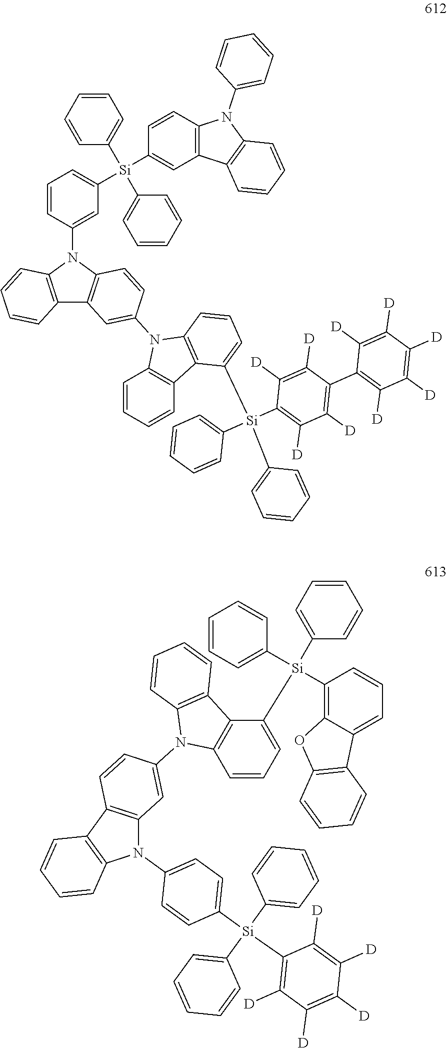 [SMILES: [2H]c1c([2H])c([2H])c(-c2c([2H])c([2H])c([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)c([2H])c2[2H])c([2H])c1[2H] ; [2H]c1c([2H])c([2H])c([Si](c2ccccc2)(c2ccccc2)c2ccc(-n3c4ccccc4c4ccc(-n5c6ccccc6c6c([Si](c7ccccc7)(c7ccccc7)c7cccc8c7oc7ccccc78)cccc65)cc43)cc2)c([2H])c1[2H]]

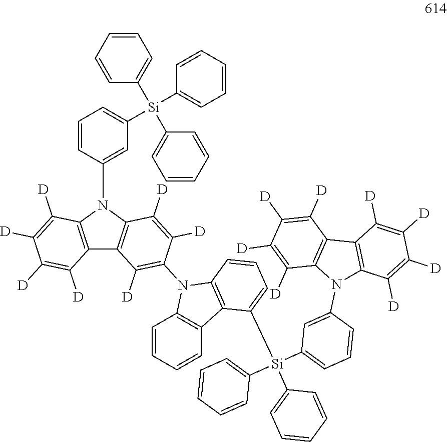 [SMILES: [2H]c1c([2H])c([2H])c2c(c1[2H])c1c([2H])c([2H])c([2H])c([2H])c1n2-c1cccc([Si](c2ccccc2)(c2ccccc2)c2cccc3c2c2ccccc2n3-c2c([2H])c([2H])c3c(c2[2H])c2c([2H])c([2H])c([2H])c([2H])c2n3-c2cccc([Si](c3ccccc3)(c3ccccc3)c3ccccc3)c2)c1]

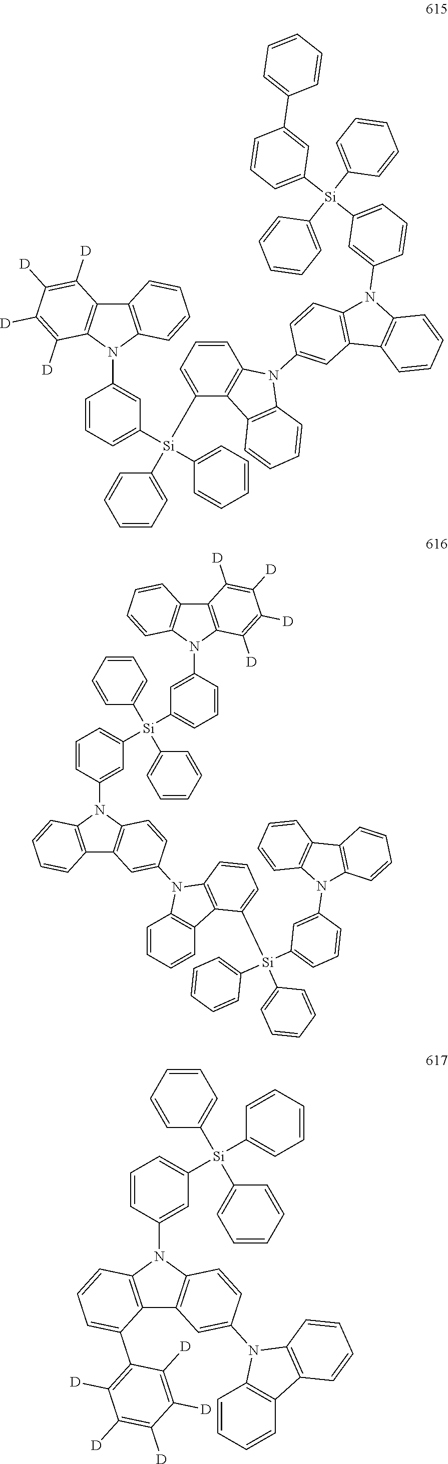 [SMILES: [2H]c1c([2H])c([2H])c2c(c1[2H])c1ccccc1n2-c1cccc([Si](c2ccccc2)(c2ccccc2)c2cccc3c2c2ccccc2n3-c2ccc3c(c2)c2ccccc2n3-c2cccc([Si](c3ccccc3)(c3ccccc3)c3cccc(-c4ccccc4)c3)c2)c1 ; [2H]c1c([2H])c([2H])c2c(c1[2H])c1ccccc1n2-c1cccc([Si](c2ccccc2)(c2ccccc2)c2cccc(-n3c4ccccc4c4cc(-n5c6ccccc6c6c([Si](c7ccccc7)(c7ccccc7)c7cccc(-n8c9ccccc9c9ccccc98)c7)cccc65)ccc43)c2)c1 ; [2H]c1c([2H])c([2H])c(-c2cccc3c2c2cc(-n4c5ccccc5c5ccccc54)ccc2n3-c2cccc([Si](c3ccccc3)(c3ccccc3)c3ccccc3)c2)c([2H])c1[2H]]

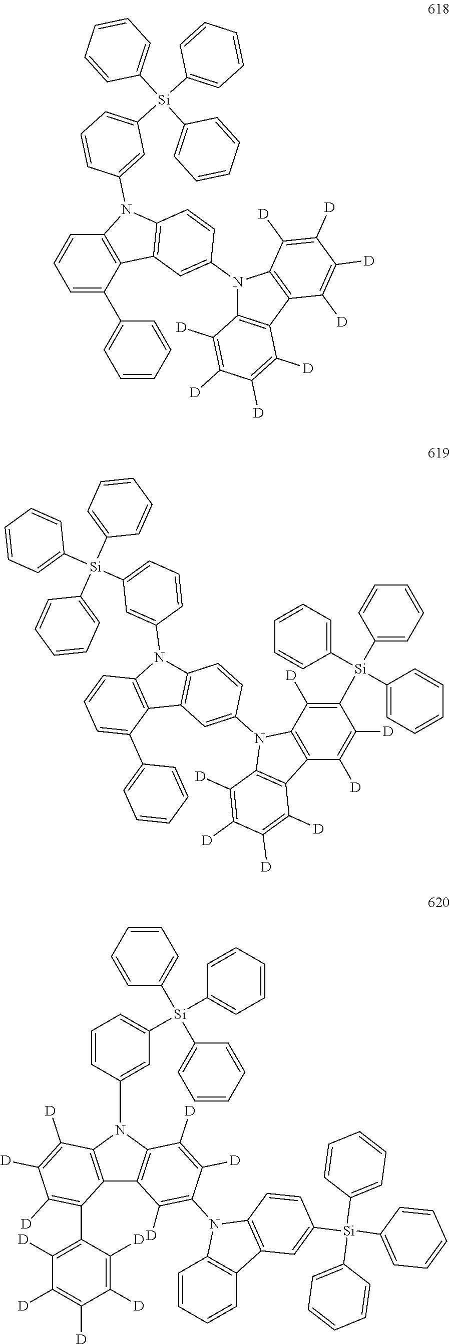

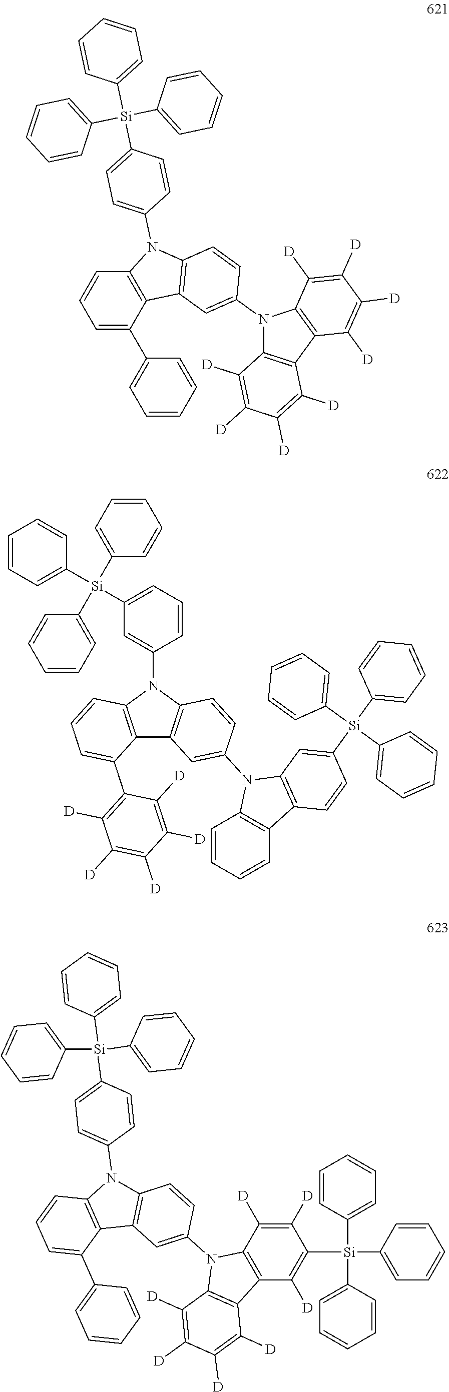 [SMILES: [2H]c1c([2H])c([2H])c2c(c1[2H])c1c([2H])c([2H])c([2H])c([2H])c1n2-c1ccc2c(c1)c1c(-c3ccccc3)cccc1n2-c1ccc([Si](c2ccccc2)(c2ccccc2)c2ccccc2)cc1 ; [2H]c1c([2H])c([2H])c(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccccc3)c2)c([2H])c1[2H] ; [2H]c1c([2H])c([2H])c2c(c1[2H])c1c([2H])c([Si](c3ccccc3)(c3ccccc3)c3ccccc3)c([2H])c([2H])c1n2-c1ccc2c(c1)c1c(-c3ccccc3)cccc1n2-c1ccc([Si](c2ccccc2)(c2ccccc2)c2ccccc2)cc1]

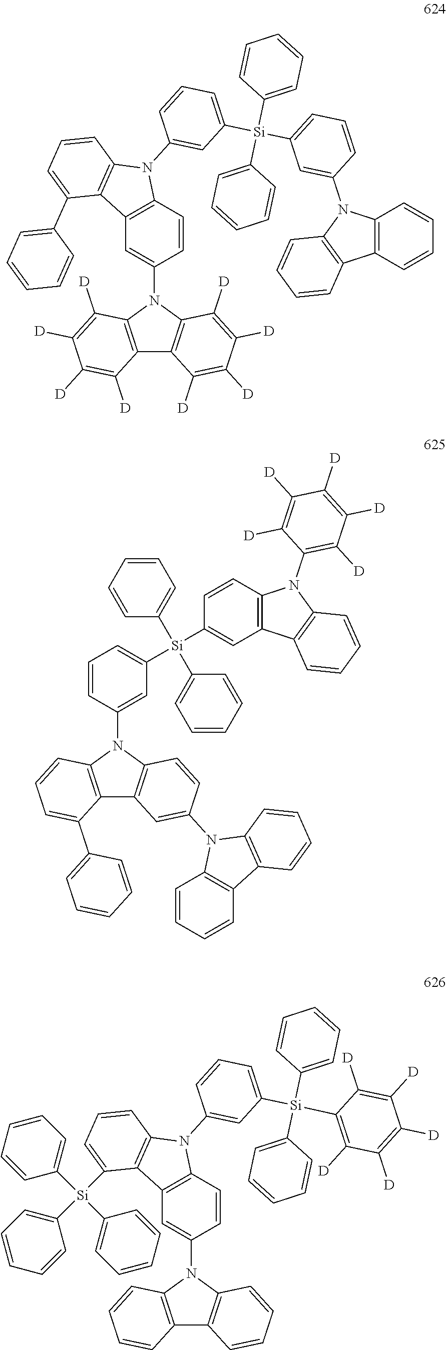 [SMILES: [2H]c1c([2H])c([2H])c2c(c1[2H])c1c([2H])c([2H])c([2H])c([2H])c1n2-c1ccc2c(c1)c1c(-c3ccccc3)cccc1n2-c1cccc([Si](c2ccccc2)(c2ccccc2)c2cccc(-n3c4ccccc4c4ccccc43)c2)c1 ; [2H]c1c([2H])c([2H])c(-n2c3ccccc3c3cc([Si](c4ccccc4)(c4ccccc4)c4cccc(-n5c6ccc(-n7c8ccccc8c8ccccc87)cc6c6c(-c7ccccc7)cccc65)c4)ccc32)c([2H])c1[2H] ; [2H]c1c([2H])c([2H])c([Si](c2ccccc2)(c2ccccc2)c2cccc(-n3c4ccc(-n5c6ccccc6c6ccccc65)cc4c4c([Si](c5ccccc5)(c5ccccc5)c5ccccc5)cccc43)c2)c([2H])c1[2H]]

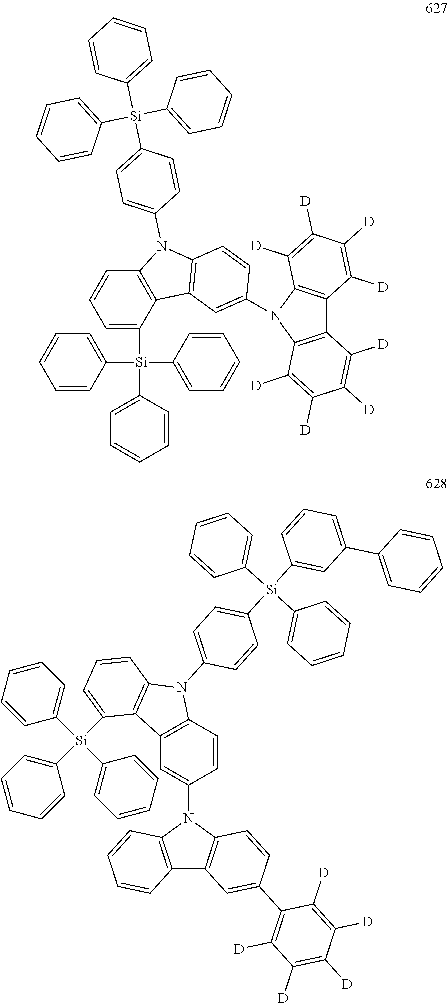 [SMILES: [2H]c1c([2H])c([2H])c2c(c1[2H])c1c([2H])c([2H])c([2H])c([2H])c1n2-c1ccc2c(c1)c1c([Si](c3ccccc3)(c3ccccc3)c3ccccc3)cccc1n2-c1ccc([Si](c2ccccc2)(c2ccccc2)c2ccccc2)cc1 ; [2H]c1c([2H])c([2H])c(-c2ccc3c(c2)c2ccccc2n3-c2ccc3c(c2)c2c([Si](c4ccccc4)(c4ccccc4)c4ccccc4)cccc2n3-c2ccc([Si](c3ccccc3)(c3ccccc3)c3cccc(-c4ccccc4)c3)cc2)c([2H])c1[2H]]

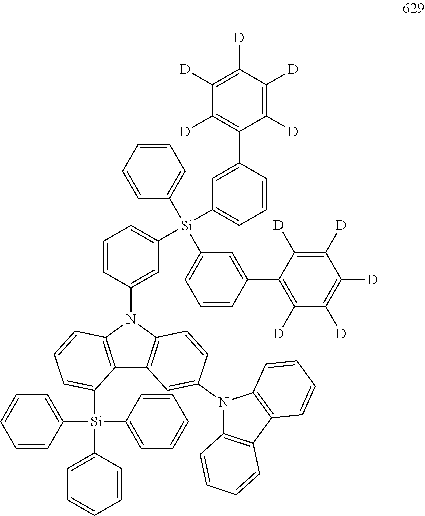 [SMILES: [2H]c1c([2H])c([2H])c(-c2cccc([Si](c3ccccc3)(c3cccc(-c4c([2H])c([2H])c([2H])c([2H])c4[2H])c3)c3cccc(-n4c5ccc(-n6c7ccccc7c7ccccc76)cc5c5c([Si](c6ccccc6)(c6ccccc6)c6ccccc6)cccc54)c3)c2)c([2H])c1[2H]]

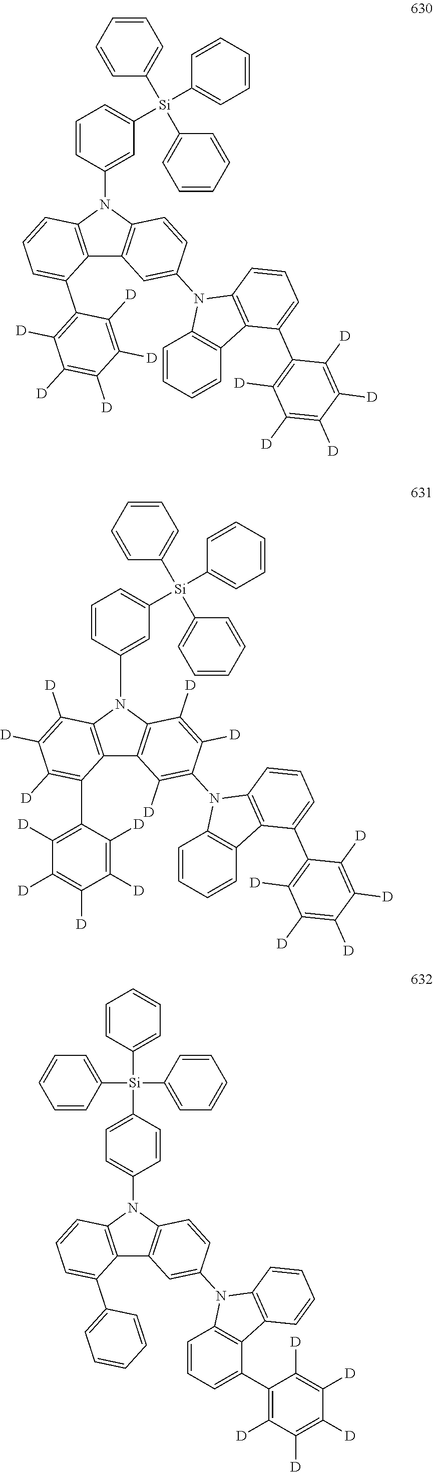 [SMILES: [2H]c1c([2H])c([2H])c(-c2cccc3c2c2ccccc2n3-c2ccc3c(c2)c2c(-c4c([2H])c([2H])c([2H])c([2H])c4[2H])cccc2n3-c2cccc([Si](c3ccccc3)(c3ccccc3)c3ccccc3)c2)c([2H])c1[2H] ; [2H]c1c([2H])c([2H])c(-c2cccc3c2c2ccccc2n3-c2c([2H])c([2H])c3c(c2[2H])c2c(-c4c([2H])c([2H])c([2H])c([2H])c4[2H])c([2H])c([2H])c([2H])c2n3-c2cccc([Si](c3ccccc3)(c3ccccc3)c3ccccc3)c2)c([2H])c1[2H] ; [2H]c1c([2H])c([2H])c(-c2cccc3c2c2ccccc2n3-c2ccc3c(c2)c2c(-c4ccccc4)cccc2n3-c2ccc([Si](c3ccccc3)(c3ccccc3)c3ccccc3)cc2)c([2H])c1[2H]]

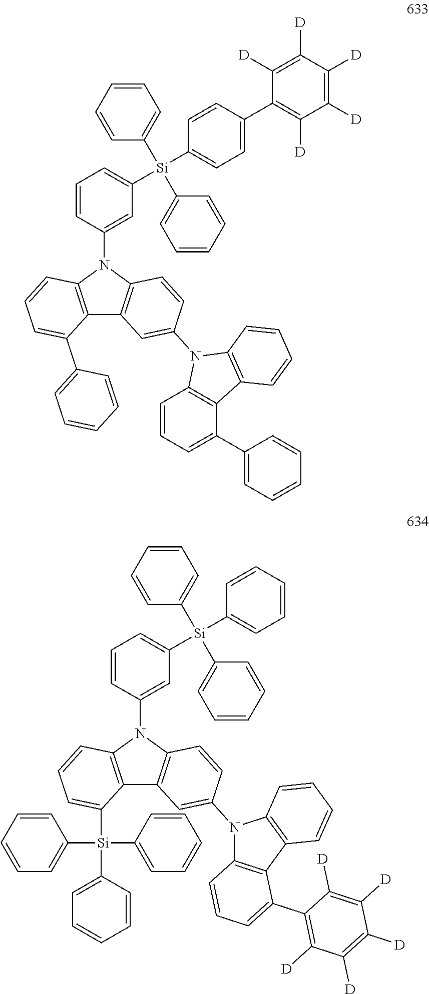 [SMILES: [2H]c1c([2H])c([2H])c(-c2ccc([Si](c3ccccc3)(c3ccccc3)c3cccc(-n4c5ccc(-n6c7ccccc7c7c(-c8ccccc8)cccc76)cc5c5c(-c6ccccc6)cccc54)c3)cc2)c([2H])c1[2H] ; [2H]c1c([2H])c([2H])c(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)c([2H])c1[2H]]

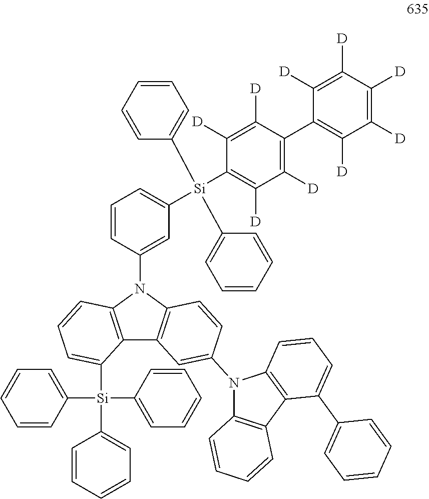 [SMILES: [2H]c1c([2H])c([2H])c(-c2c([2H])c([2H])c([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c([2H])c2[2H])c([2H])c1[2H]]

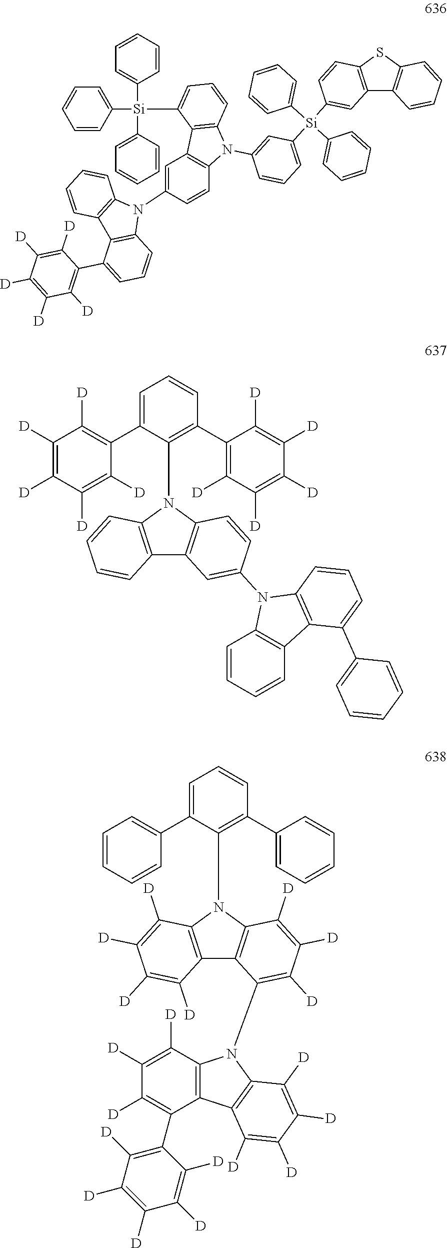 [SMILES: [2H]c1c([2H])c([2H])c(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4sc5ccccc5c4c3)c2)c([2H])c1[2H] ; [2H]c1c([2H])c([2H])c(-c2cccc(-c3c([2H])c([2H])c([2H])c([2H])c3[2H])c2-n2c3ccccc3c3cc(-n4c5ccccc5c5c(-c6ccccc6)cccc54)ccc32)c([2H])c1[2H] ; [2H]c1c([2H])c([2H])c(-c2c([2H])c([2H])c([2H])c3c2c2c([2H])c([2H])c([2H])c([2H])c2n3-c2c([2H])c([2H])c([2H])c3c2c2c([2H])c([2H])c([2H])c([2H])c2n3-c2c(-c3ccccc3)cccc2-c2ccccc2)c([2H])c1[2H]]

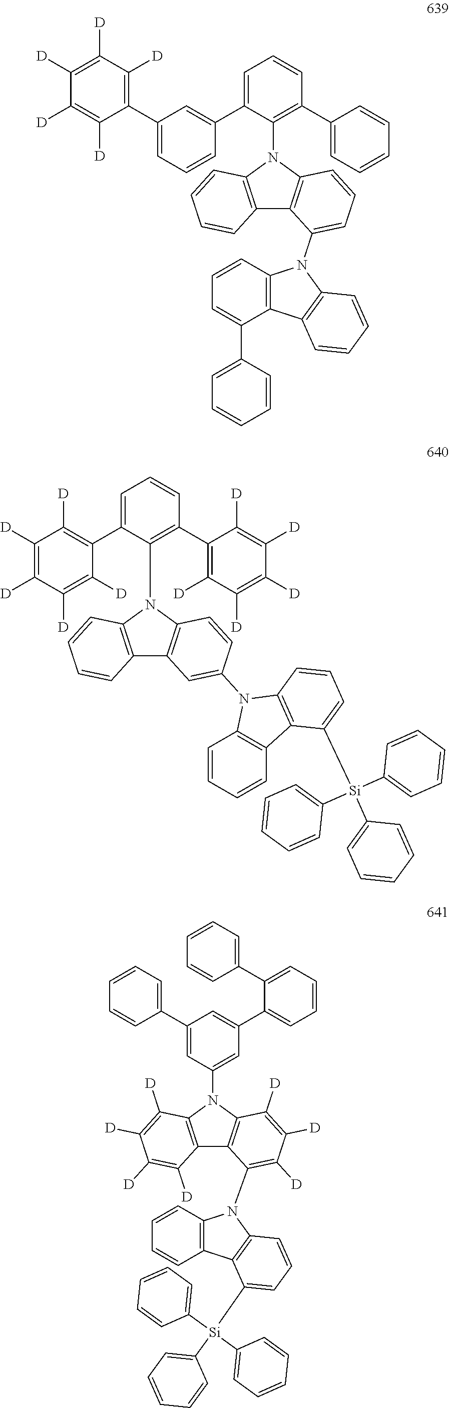 [SMILES: [2H]c1c([2H])c([2H])c(-c2cccc(-c3cccc(-c4ccccc4)c3-n3c4ccccc4c4c(-n5c6ccccc6c6c(-c7ccccc7)cccc65)cccc43)c2)c([2H])c1[2H] ; [2H]c1c([2H])c([2H])c(-c2cccc(-c3c([2H])c([2H])c([2H])c([2H])c3[2H])c2-n2c3ccccc3c3cc(-n4c5ccccc5c5c([Si](c6ccccc6)(c6ccccc6)c6ccccc6)cccc54)ccc32)c([2H])c1[2H] ; [2H]c1c([2H])c([2H])c2c(c1[2H])c1c(-n3c4ccccc4c4c([Si](c5ccccc5)(c5ccccc5)c5ccccc5)cccc43)c([2H])c([2H])c([2H])c1n2-c1cc(-c2ccccc2)cc(-c2ccccc2-c2ccccc2)c1]

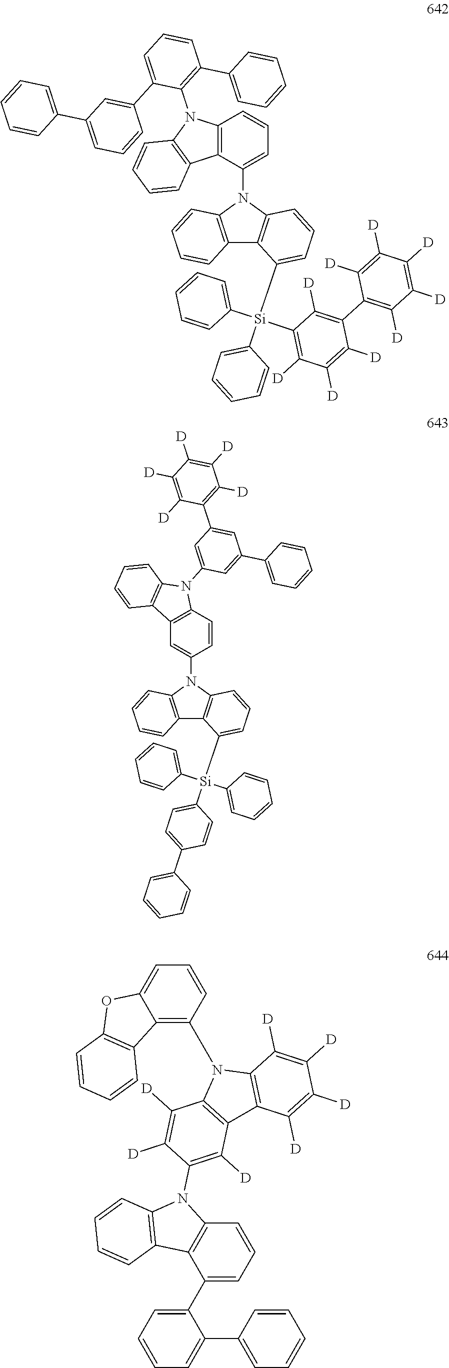 [SMILES: [2H]c1c([2H])c([2H])c(-c2c([2H])c([2H])c([2H])c([Si](c3ccccc3)(c3ccccc3)c3cccc4c3c3ccccc3n4-c3cccc4c3c3ccccc3n4-c3c(-c4ccccc4)cccc3-c3cccc(-c4ccccc4)c3)c2[2H])c([2H])c1[2H] ; [2H]c1c([2H])c([2H])c(-c2cc(-c3ccccc3)cc(-n3c4ccccc4c4cc(-n5c6ccccc6c6c([Si](c7ccccc7)(c7ccccc7)c7ccc(-c8ccccc8)cc7)cccc65)ccc43)c2)c([2H])c1[2H] ; [2H]c1c([2H])c([2H])c2c(c1[2H])c1c([2H])c(-n3c4ccccc4c4c(-c5ccccc5-c5ccccc5)cccc43)c([2H])c([2H])c1n2-c1cccc2oc3ccccc3c12]

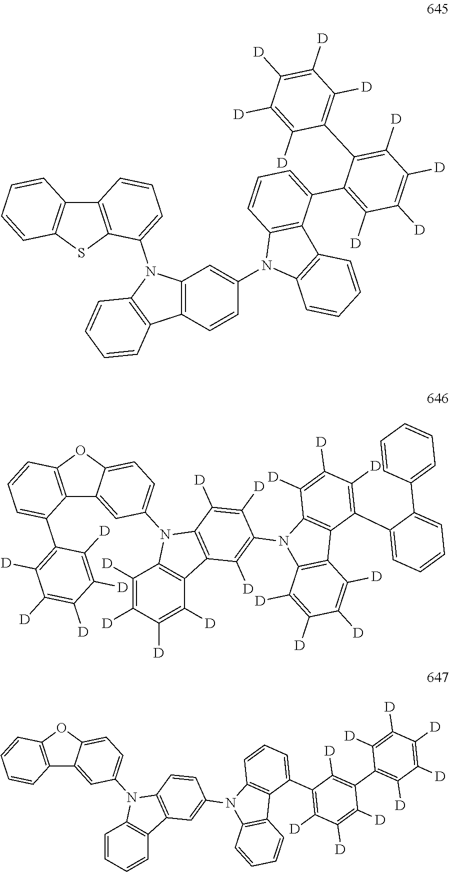 [SMILES: [2H]c1c([2H])c([2H])c(-c2c([2H])c([2H])c([2H])c([2H])c2-c2cccc3c2c2ccccc2n3-c2ccc3c4ccccc4n(-c4cccc5c4sc4ccccc45)c3c2)c([2H])c1[2H] ; [2H]c1c([2H])c([2H])c(-c2cccc3oc4ccc(-n5c6c([2H])c([2H])c([2H])c([2H])c6c6c([2H])c(-n7c8c([2H])c([2H])c([2H])c([2H])c8c8c(-c9ccccc9-c9ccccc9)c([2H])c([2H])c([2H])c87)c([2H])c([2H])c65)cc4c23)c([2H])c1[2H] ; [2H]c1c([2H])c([2H])c(-c2c([2H])c([2H])c([2H])c(-c3cccc4c3c3ccccc3n4-c3ccc4c(c3)c3ccccc3n4-c3ccc4oc5ccccc5c4c3)c2[2H])c([2H])c1[2H]]

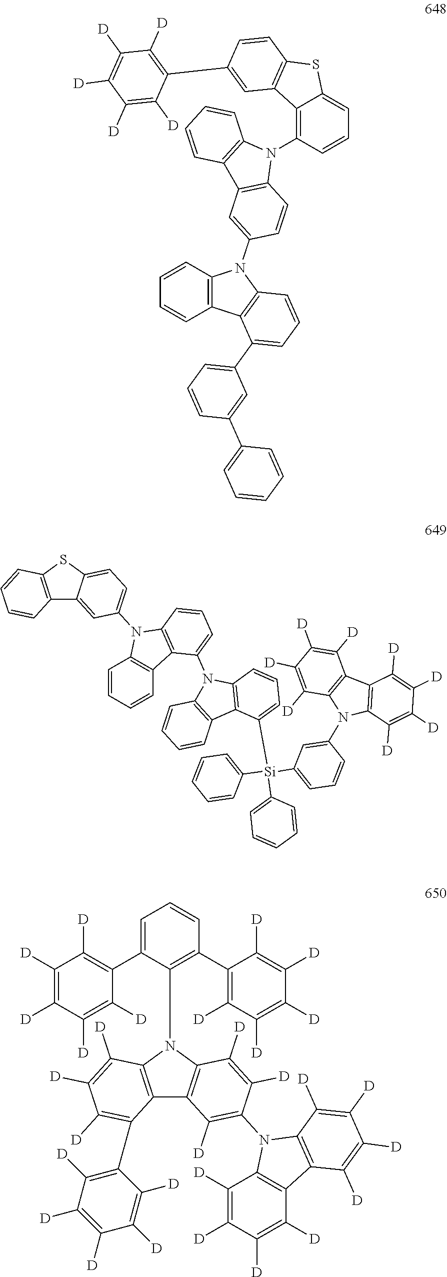

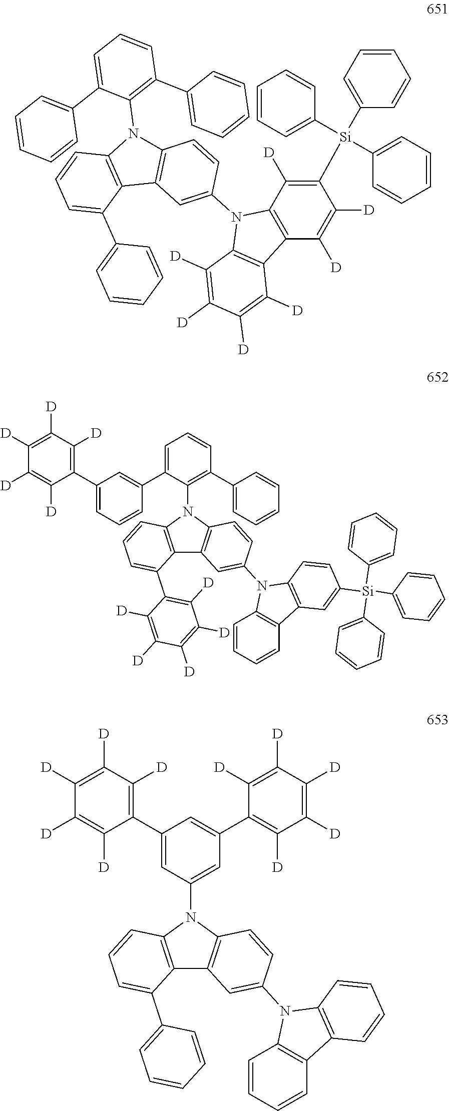 [SMILES: [2H]c1c([2H])c([2H])c2c(c1[2H])c1c([2H])c([2H])c([Si](c3ccccc3)(c3ccccc3)c3ccccc3)c([2H])c1n2-c1ccc2c(c1)c1c(-c3ccccc3)cccc1n2-c1c(-c2ccccc2)cccc1-c1ccccc1 ; [2H]c1c([2H])c([2H])c(-c2cccc(-c3cccc(-c4ccccc4)c3-n3c4ccc(-n5c6ccccc6c6cc([Si](c7ccccc7)(c7ccccc7)c7ccccc7)ccc65)cc4c4c(-c5c([2H])c([2H])c([2H])c([2H])c5[2H])cccc43)c2)c([2H])c1[2H] ; [2H]c1c([2H])c([2H])c(-c2cc(-c3c([2H])c([2H])c([2H])c([2H])c3[2H])cc(-n3c4ccc(-n5c6ccccc6c6ccccc65)cc4c4c(-c5ccccc5)cccc43)c2)c([2H])c1[2H]]

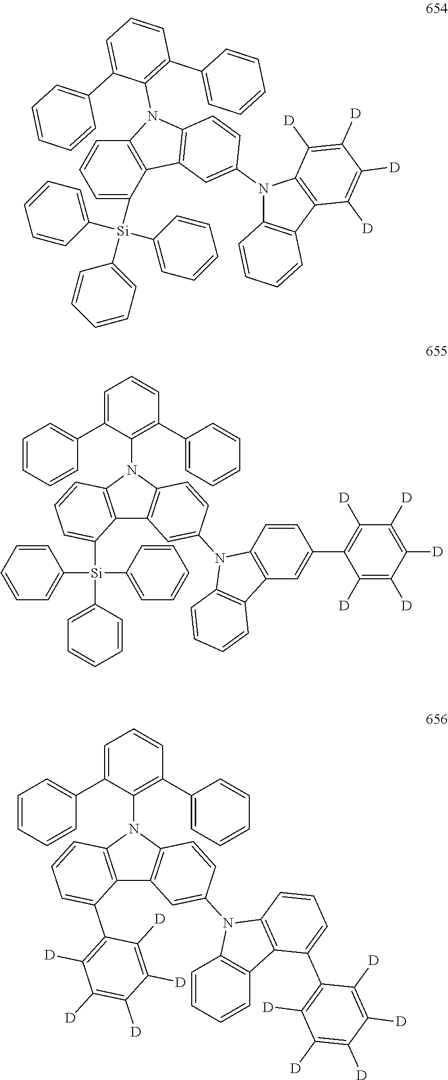 [SMILES: [2H]c1c([2H])c([2H])c2c(c1[2H])c1ccccc1n2-c1ccc2c(c1)c1c([Si](c3ccccc3)(c3ccccc3)c3ccccc3)cccc1n2-c1c(-c2ccccc2)cccc1-c1ccccc1 ; [2H]c1c([2H])c([2H])c(-c2ccc3c(c2)c2ccccc2n3-c2ccc3c(c2)c2c([Si](c4ccccc4)(c4ccccc4)c4ccccc4)cccc2n3-c2c(-c3ccccc3)cccc2-c2ccccc2)c([2H])c1[2H] ; [2H]c1c([2H])c([2H])c(-c2cccc3c2c2ccccc2n3-c2ccc3c(c2)c2c(-c4c([2H])c([2H])c([2H])c([2H])c4[2H])cccc2n3-c2c(-c3ccccc3)cccc2-c2ccccc2)c([2H])c1[2H]]

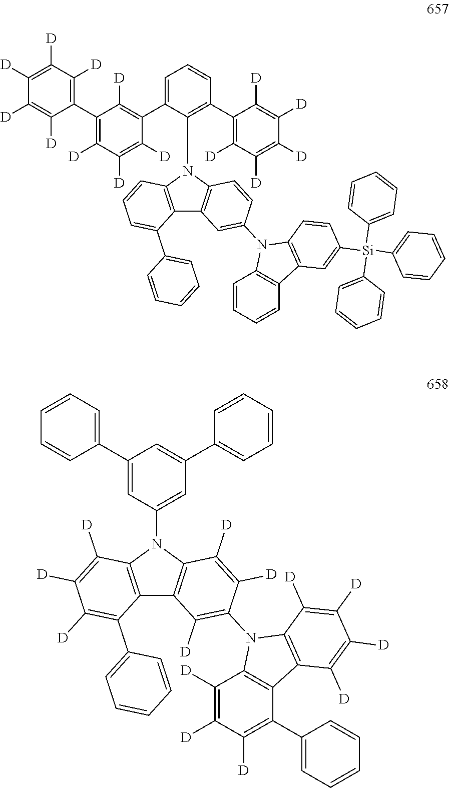 [SMILES: [2H]c1c([2H])c([2H])c(-c2cccc(-c3c([2H])c([2H])c([2H])c(-c4c([2H])c([2H])c([2H])c([2H])c4[2H])c3[2H])c2-n2c3ccc(-n4c5ccccc5c5cc([Si](c6ccccc6)(c6ccccc6)c6ccccc6)ccc54)cc3c3c(-c4ccccc4)cccc32)c([2H])c1[2H] ; [2H]c1c([2H])c([2H])c2c(c1[2H])c1c(-c3ccccc3)c([2H])c([2H])c([2H])c1n2-c1c([2H])c([2H])c2c(c1[2H])c1c(-c3ccccc3)c([2H])c([2H])c([2H])c1n2-c1cc(-c2ccccc2)cc(-c2ccccc2)c1]

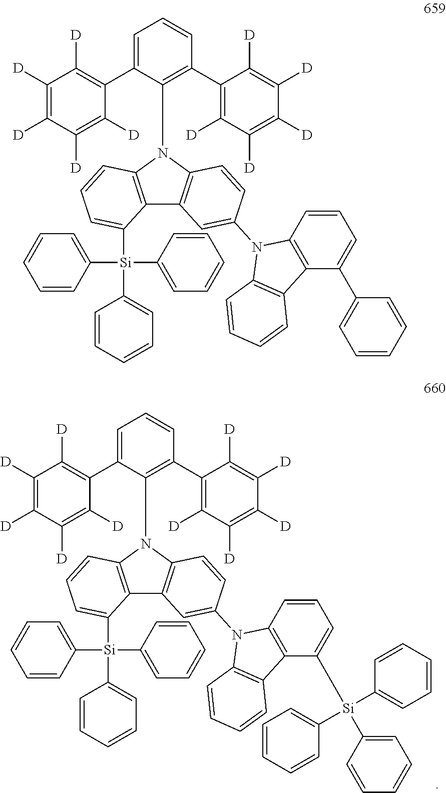 [SMILES: [2H]c1c([2H])c([2H])c(-c2cccc(-c3c([2H])c([2H])c([2H])c([2H])c3[2H])c2-n2c3ccc(-n4c5ccccc5c5c(-c6ccccc6)cccc54)cc3c3c([Si](c4ccccc4)(c4ccccc4)c4ccccc4)cccc32)c([2H])c1[2H] ; [2H]c1c([2H])c([2H])c(-c2cccc(-c3c([2H])c([2H])c([2H])c([2H])c3[2H])c2-n2c3ccc(-n4c5ccccc5c5c([Si](c6ccccc6)(c6ccccc6)c6ccccc6)cccc54)cc3c3c([Si](c4ccccc4)(c4ccccc4)c4ccccc4)cccc32)c([2H])c1[2H]]

11. The light emitting device of claim 1, wherein the emission layer further comprises a second compound represented by Formula ET-1: 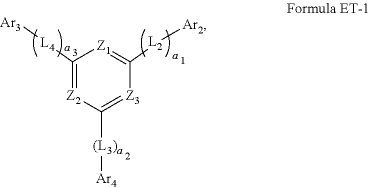
wherein in Formula ET-1,
at least one among Z₁ to Z₃ is N, and remaining ones of Z₁ to Z₃ are CR₄₆,
R₄₆ is a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms,
a₁ to a₃ are each independently an integer of 0 to 10,
L₂ to L₄ are each independently a direct linkage, a substituted or unsubstituted arylene group of 6 to 30 ring-forming carbon atoms, or a substituted or unsubstituted heteroarylene group of 2 to 30 ring-forming carbon atoms,
when any of a1 to as is an integer of 2 or more, corresponding L₂ to L₄ are each independently a substituted or unsubstituted arylene group of 6 to 30 ring-forming carbon atoms, or a substituted or unsubstituted heteroarylene group of 2 to 30 ring-forming carbon atoms, and
Ar₂ to Ar₄ are each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

12. The light emitting device of claim 1, wherein the emission layer further comprises a third compound represented by Formula D-1: 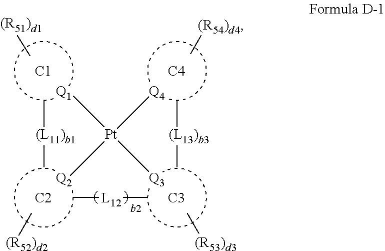
wherein in Formula D-1,
Q₁ to Q₄ are each independently C or N,
C1 to C4 are each independently a substituted or unsubstituted hydrocarbon ring of 5 to 30 ring-forming carbon atoms, or a substituted or unsubstituted heterocycle of 2 to 30 ring-forming carbon atoms,
L₁₁ to L₁₃ are each independently a direct linkage, 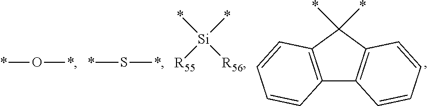
a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms,
b1 to b3 are each independently 0 or 1,
R₅₁ to R₅₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms, and/or combined with an adjacent group to form a ring, and
d1 to d4 are each independently an integer of 0 to 4.

13. A nitrogen-containing compound represented by Formula 1: 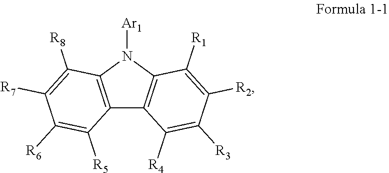
wherein in Formula 1,
Ar₁ is a substituted or unsubstituted aryl group of 6 to 30 ring-forming carbon atoms, or a substituted or unsubstituted heteroaryl group of 2 to 30 ring-forming carbon atoms,
R₁ to R₈ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
any one among R₅ to R₈ is a substituent represented by Formula 2: 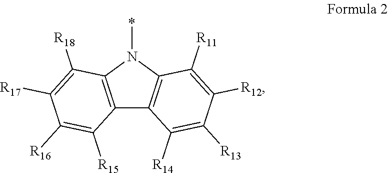
wherein in Formula 2,
R₁₁ to R₁₈ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
in Formula 1 and Formula 2, at least one of R₄ or R₁₄ is a substituted or unsubstituted phenyl group or a substituent represented by Formula 3: 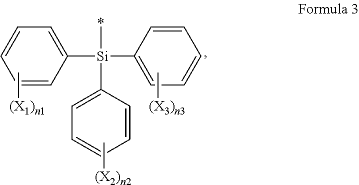
and
wherein in Formula 3,
X₁ to X₃ are each independently a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and
n1 to n3 are each independently an integer of 0 to 5.

14. The nitrogen-containing compound of claim 13, wherein the nitrogen-containing compound represented by Formula 1 is represented by Formula 1-1: 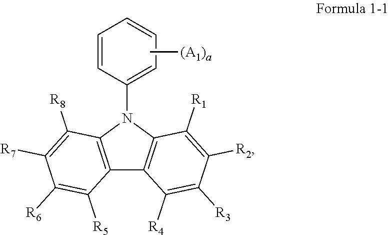
and
wherein in Formula 1-1,
A₁ is a hydrogen atom, a deuterium atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl, or a substituted or unsubstituted biphenyl group, and/or combined with an adjacent group to form a ring,
“a” is an integer of 0 to 5, and
R₁ to R₈ are as defined in Formula 1.

15. The nitrogen-containing compound of claim 14, wherein, in Formula 1-1, A₁ is a substituent represented by any one among Formula 4-1 to Formula 4-11: 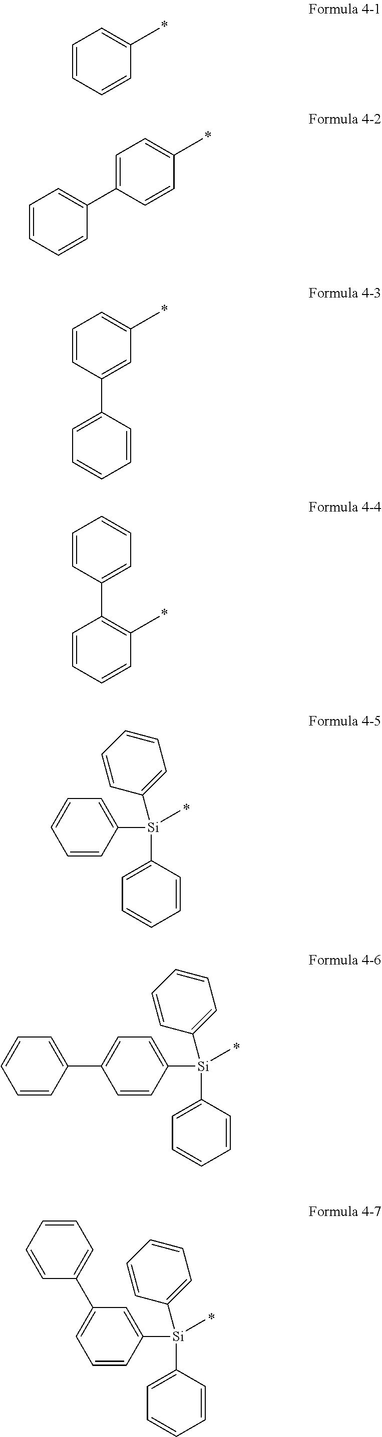 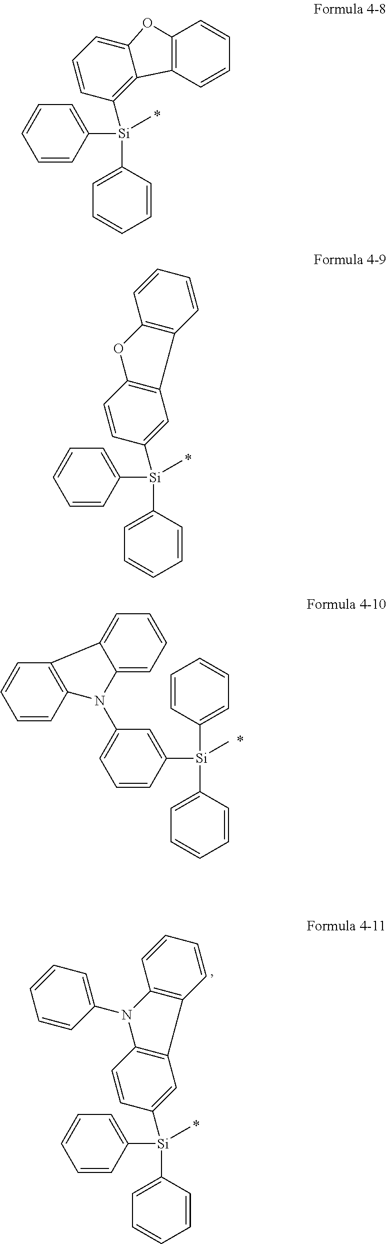
and
wherein in Formula 4-1 to Formula 4-11,
“ [symbol] ” is a position connected with Formula 1-1.

16. The nitrogen-containing compound of claim 13, wherein the nitrogen-containing compound represented by Formula 1 is represented by Formula 1-2: 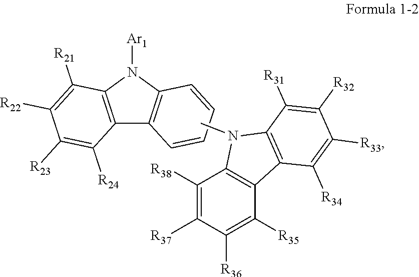
and
wherein in Formula 1-2,
R₂₁ to R₃₈ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
at least one of R₂₄ or R₃4 is a substituted or unsubstituted phenyl group, or a substituent represented by Formula 3, and
Ar₁ is as defined in Formula 1.

17. The nitrogen-containing compound of claim 13, wherein the substituent represented by Formula 3 is represented by Formula 3-1: 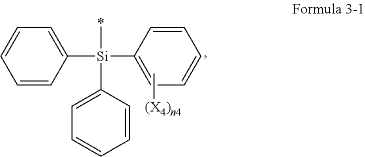
and
wherein in Formula 3-1,
X₄ is a hydrogen atom, a deuterium atom, a cyano group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted phenyl group, or a substituted or unsubstituted heteroaryl group of 2 to 10 ring-forming carbon atoms, and/or combined with an adjacent group to form a ring, and
n4 is an integer of 0 to 5.

18. The nitrogen-containing compound of claim 13, wherein the nitrogen-containing compound represented by Formula 1 is represented by Formula 1-3 or Formula 1-4: 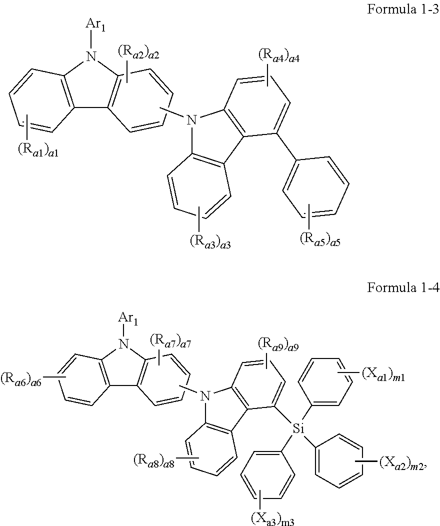
and
wherein in Formula 1-3 and Formula 1-4,
Rₐ₁ to Rₐ₉ are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Xₐ₁ to Xₐ₃ are each independently a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
a1, a3, a6 and a8 are each independently an integer of 0 to 4,
a2, a4, a7 and a9 are each independently an integer of 0 to 3,
a5 and m1 to m3 are each independently an integer of 0 to 5, and
Ar₁ is the same as defined in Formula 1.

19. The nitrogen-containing compound of claim 13, wherein the nitrogen-containing compound represented by Formula 1 is represented by Formula 1-5 or Formula 1-6: 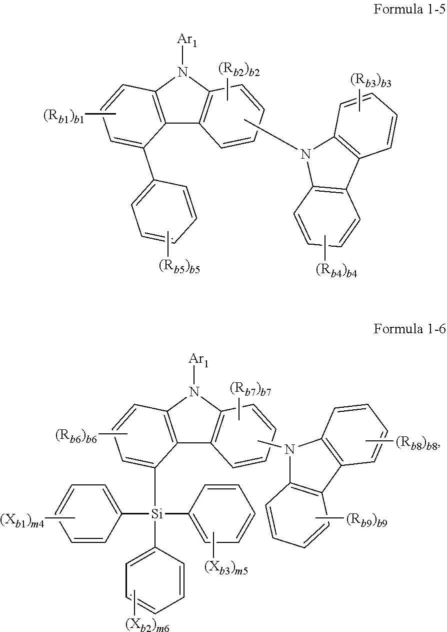
and
wherein in Formula 1-5 and Formula 1-6,
R_{b1} to R_{b9} are each independently a hydrogen atom, a deuterium atom, a halogen atom, a cyano group, a substituted or unsubstituted silyl group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
X_{b1} to X_{b3} are each independently a hydrogen atom, a deuterium atom, a halogen atom, a cyano group, a substituted or unsubstituted silyl group, a substituted or unsubstituted thio group, a substituted or unsubstituted amine group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
b1, b2, b6 and b7 are each independently an integer of 0 to 3,
b3, b4, b8 and b9 are each independently an integer of 0 to 4,
b5 and m4 to m6 are each independently an integer of 0 to 5, and
Ar₁ is as defined in Formula 1.

20. The nitrogen-containing compound of claim 13, wherein the nitrogen-containing compound represented by Formula 1 comprises at least one among compounds represented in Compound Group 1: 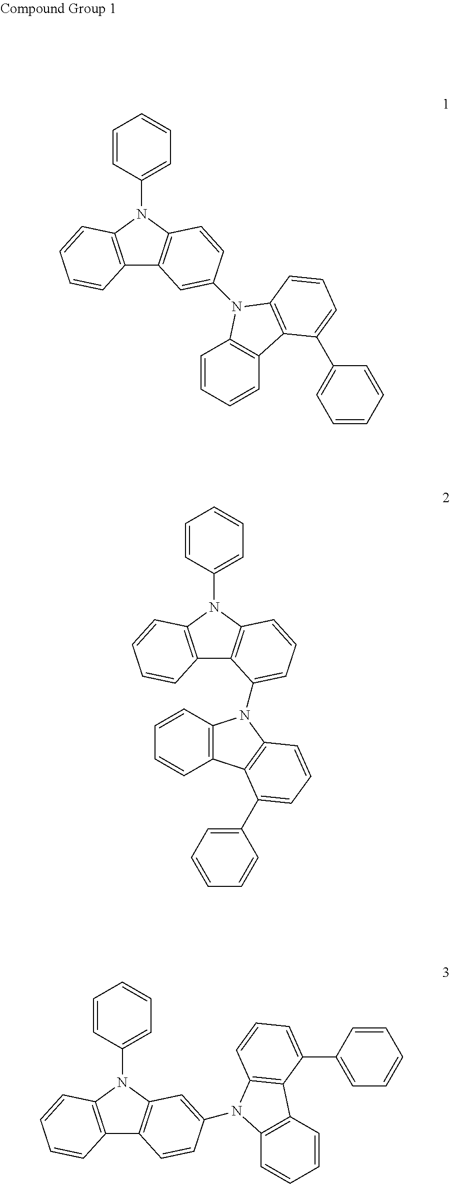 [SMILES: c1ccc(-c2cccc3c2c2ccccc2n3-c2ccc3c(c2)c2ccccc2n3-c2ccccc2)cc1 ; c1ccc(-c2cccc3c2c2ccccc2n3-c2cccc3c2c2ccccc2n3-c2ccccc2)cc1 ; c1ccc(-c2cccc3c2c2ccccc2n3-c2ccc3c4ccccc4n(-c4ccccc4)c3c2)cc1] 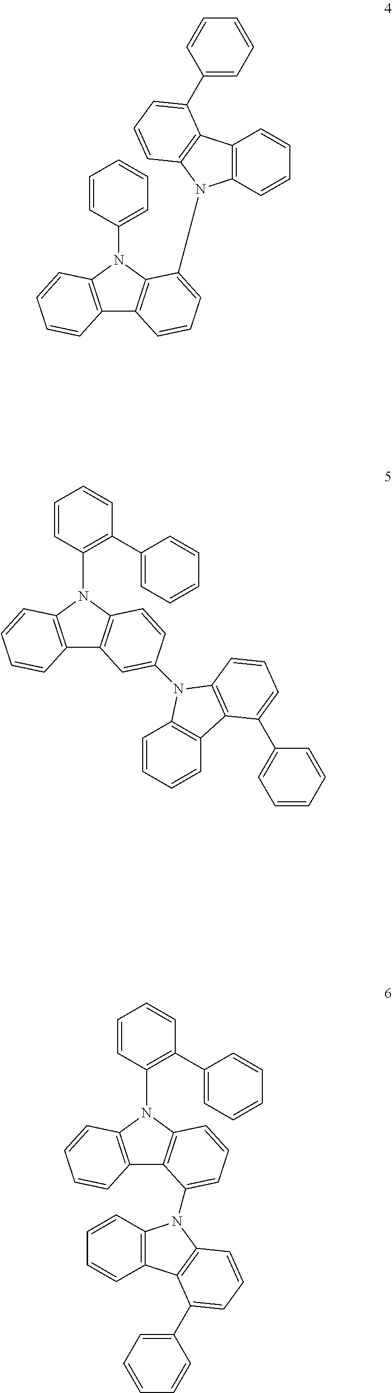 [SMILES: c1ccc(-c2cccc3c2c2ccccc2n3-c2cccc3c4ccccc4n(-c4ccccc4)c23)cc1 ; c1ccc(-c2ccccc2-n2c3ccccc3c3cc(-n4c5ccccc5c5c(-c6ccccc6)cccc54)ccc32)cc1 ; c1ccc(-c2ccccc2-n2c3ccccc3c3c(-n4c5ccccc5c5c(-c6ccccc6)cccc54)cccc32)cc1] 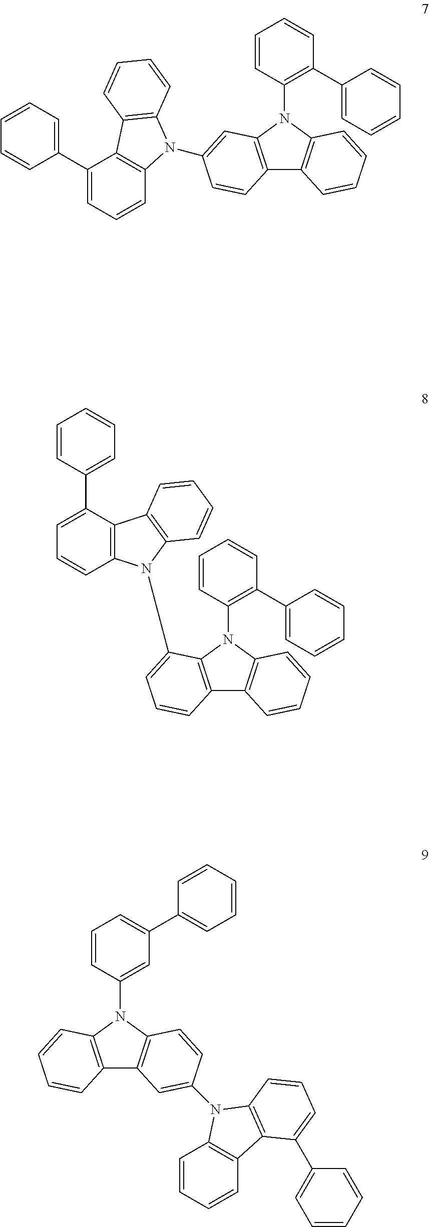 [SMILES: c1ccc(-c2ccccc2-n2c3ccccc3c3ccc(-n4c5ccccc5c5c(-c6ccccc6)cccc54)cc32)cc1 ; c1ccc(-c2ccccc2-n2c3ccccc3c3cccc(-n4c5ccccc5c5c(-c6ccccc6)cccc54)c32)cc1 ; c1ccc(-c2cccc(-n3c4ccccc4c4cc(-n5c6ccccc6c6c(-c7ccccc7)cccc65)ccc43)c2)cc1] 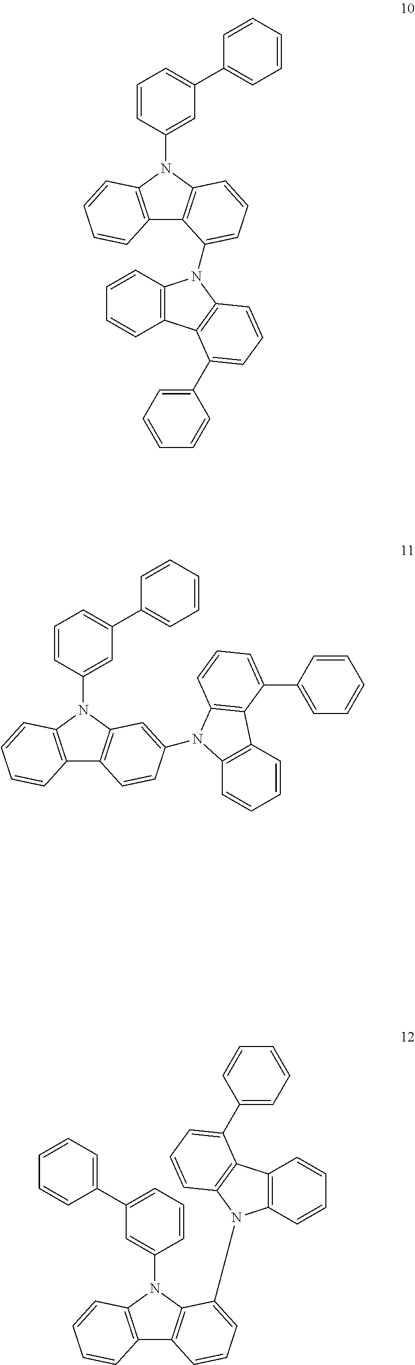 [SMILES: c1ccc(-c2cccc(-n3c4ccccc4c4c(-n5c6ccccc6c6c(-c7ccccc7)cccc65)cccc43)c2)cc1 ; c1ccc(-c2cccc(-n3c4ccccc4c4ccc(-n5c6ccccc6c6c(-c7ccccc7)cccc65)cc43)c2)cc1 ; c1ccc(-c2cccc(-n3c4ccccc4c4cccc(-n5c6ccccc6c6c(-c7ccccc7)cccc65)c43)c2)cc1] 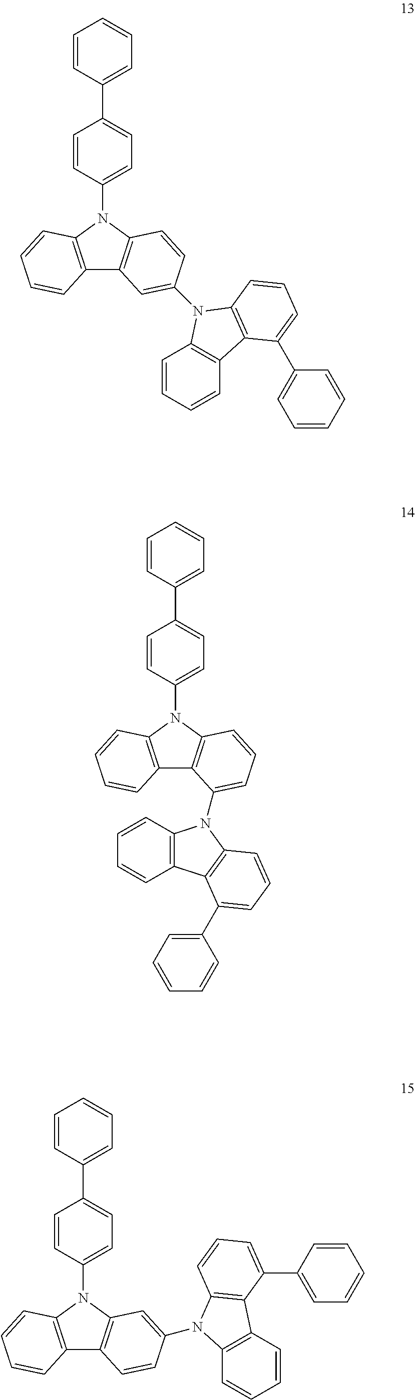 [SMILES: c1ccc(-c2ccc(-n3c4ccccc4c4cc(-n5c6ccccc6c6c(-c7ccccc7)cccc65)ccc43)cc2)cc1 ; c1ccc(-c2ccc(-n3c4ccccc4c4c(-n5c6ccccc6c6c(-c7ccccc7)cccc65)cccc43)cc2)cc1 ; c1ccc(-c2ccc(-n3c4ccccc4c4ccc(-n5c6ccccc6c6c(-c7ccccc7)cccc65)cc43)cc2)cc1] 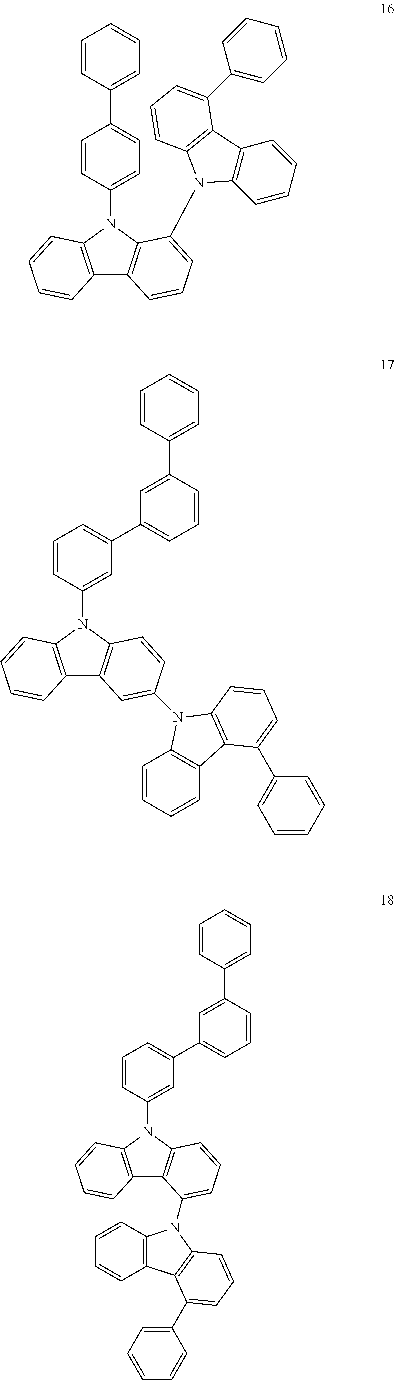 [SMILES: c1ccc(-c2ccc(-n3c4ccccc4c4cccc(-n5c6ccccc6c6c(-c7ccccc7)cccc65)c43)cc2)cc1 ; c1ccc(-c2cccc(-c3cccc(-n4c5ccccc5c5cc(-n6c7ccccc7c7c(-c8ccccc8)cccc76)ccc54)c3)c2)cc1 ; c1ccc(-c2cccc(-c3cccc(-n4c5ccccc5c5c(-n6c7ccccc7c7c(-c8ccccc8)cccc76)cccc54)c3)c2)cc1] 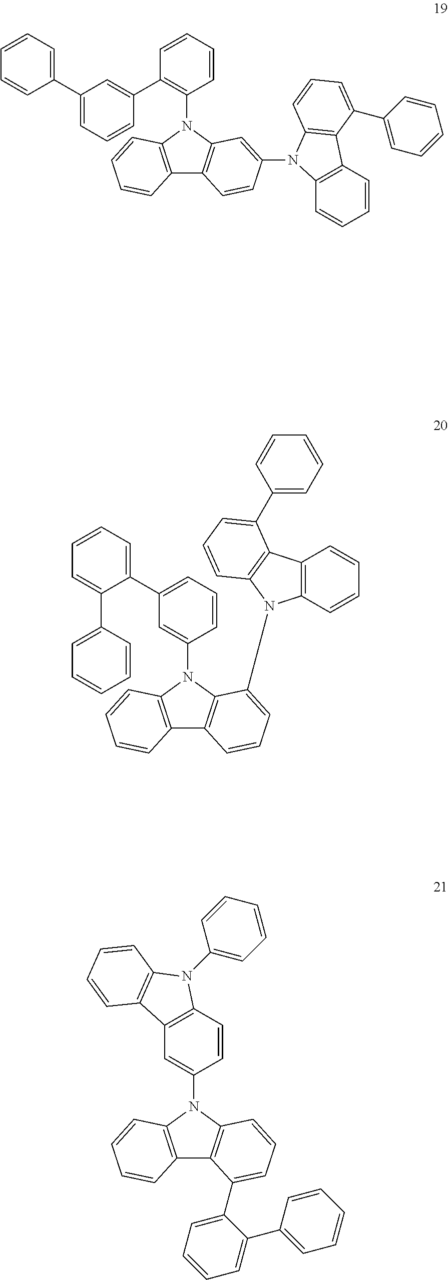 [SMILES: c1ccc(-c2cccc(-c3ccccc3-n3c4ccccc4c4ccc(-n5c6ccccc6c6c(-c7ccccc7)cccc65)cc43)c2)cc1 ; c1ccc(-c2ccccc2-c2cccc(-n3c4ccccc4c4cccc(-n5c6ccccc6c6c(-c7ccccc7)cccc65)c43)c2)cc1 ; c1ccc(-c2ccccc2-c2cccc3c2c2ccccc2n3-c2ccc3c(c2)c2ccccc2n3-c2ccccc2)cc1] 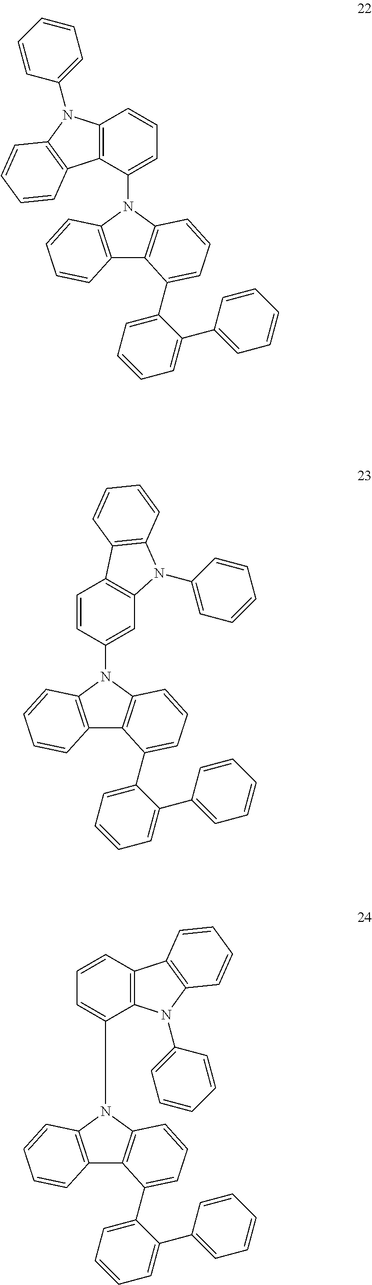 [SMILES: c1ccc(-c2ccccc2-c2cccc3c2c2ccccc2n3-c2cccc3c2c2ccccc2n3-c2ccccc2)cc1 ; c1ccc(-c2ccccc2-c2cccc3c2c2ccccc2n3-c2ccc3c4ccccc4n(-c4ccccc4)c3c2)cc1 ; c1ccc(-c2ccccc2-c2cccc3c2c2ccccc2n3-c2cccc3c4ccccc4n(-c4ccccc4)c23)cc1] 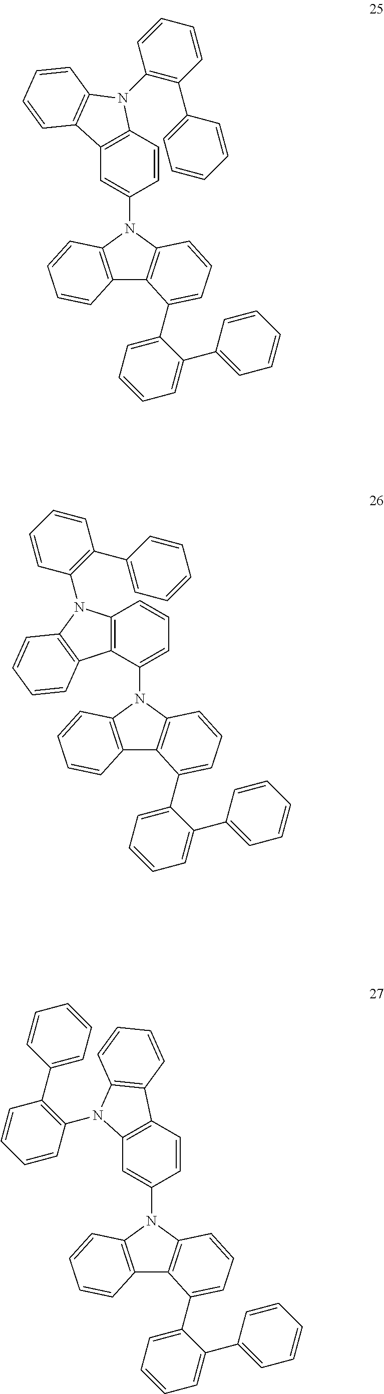 [SMILES: c1ccc(-c2ccccc2-c2cccc3c2c2ccccc2n3-c2ccc3c(c2)c2ccccc2n3-c2ccccc2-c2ccccc2)cc1 ; c1ccc(-c2ccccc2-c2cccc3c2c2ccccc2n3-c2cccc3c2c2ccccc2n3-c2ccccc2-c2ccccc2)cc1 ; c1ccc(-c2ccccc2-c2cccc3c2c2ccccc2n3-c2ccc3c4ccccc4n(-c4ccccc4-c4ccccc4)c3c2)cc1] 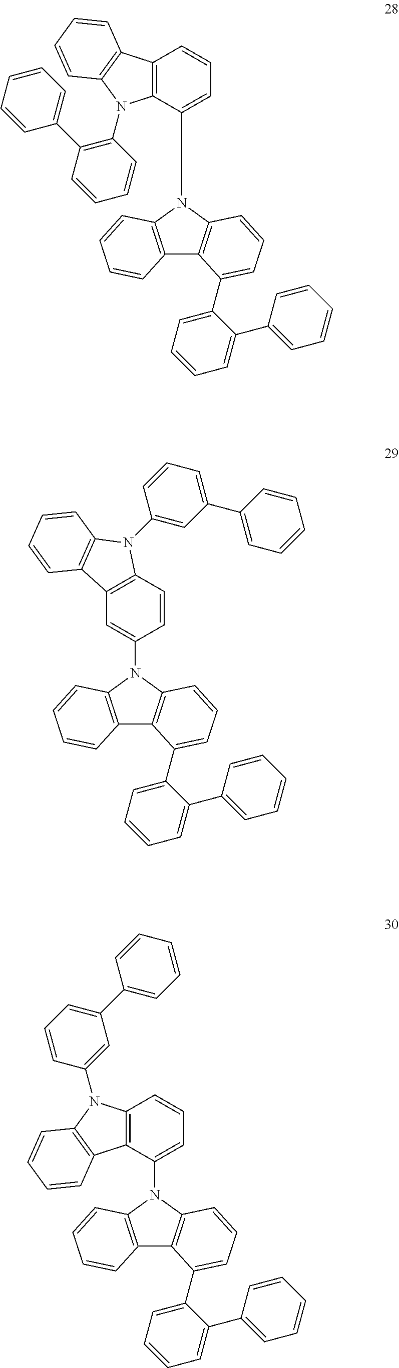 [SMILES: c1ccc(-c2ccccc2-c2cccc3c2c2ccccc2n3-c2cccc3c4ccccc4n(-c4ccccc4-c4ccccc4)c23)cc1 ; c1ccc(-c2cccc(-n3c4ccccc4c4cc(-n5c6ccccc6c6c(-c7ccccc7-c7ccccc7)cccc65)ccc43)c2)cc1 ; c1ccc(-c2cccc(-n3c4ccccc4c4c(-n5c6ccccc6c6c(-c7ccccc7-c7ccccc7)cccc65)cccc43)c2)cc1] 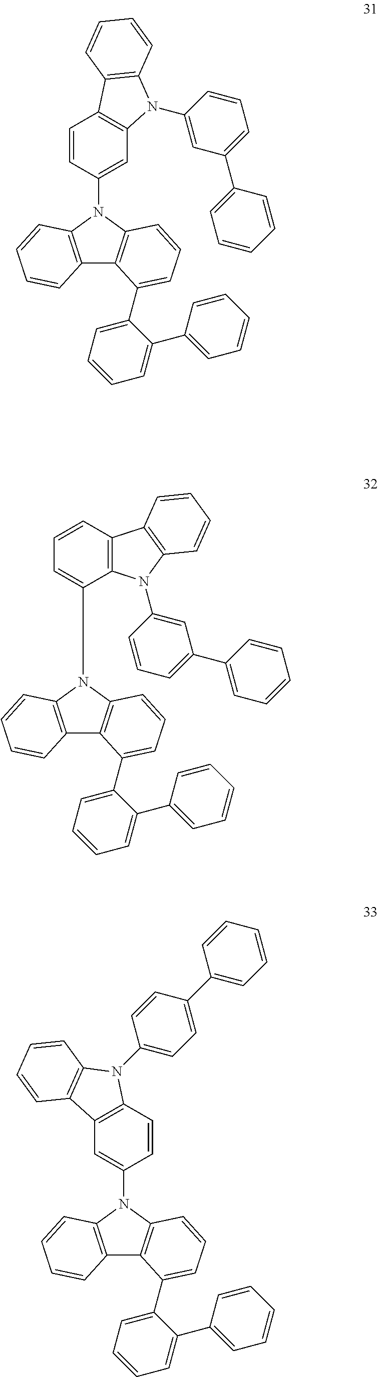 [SMILES: c1ccc(-c2cccc(-n3c4ccccc4c4ccc(-n5c6ccccc6c6c(-c7ccccc7-c7ccccc7)cccc65)cc43)c2)cc1 ; c1ccc(-c2cccc(-n3c4ccccc4c4cccc(-n5c6ccccc6c6c(-c7ccccc7-c7ccccc7)cccc65)c43)c2)cc1 ; c1ccc(-c2ccc(-n3c4ccccc4c4cc(-n5c6ccccc6c6c(-c7ccccc7-c7ccccc7)cccc65)ccc43)cc2)cc1] 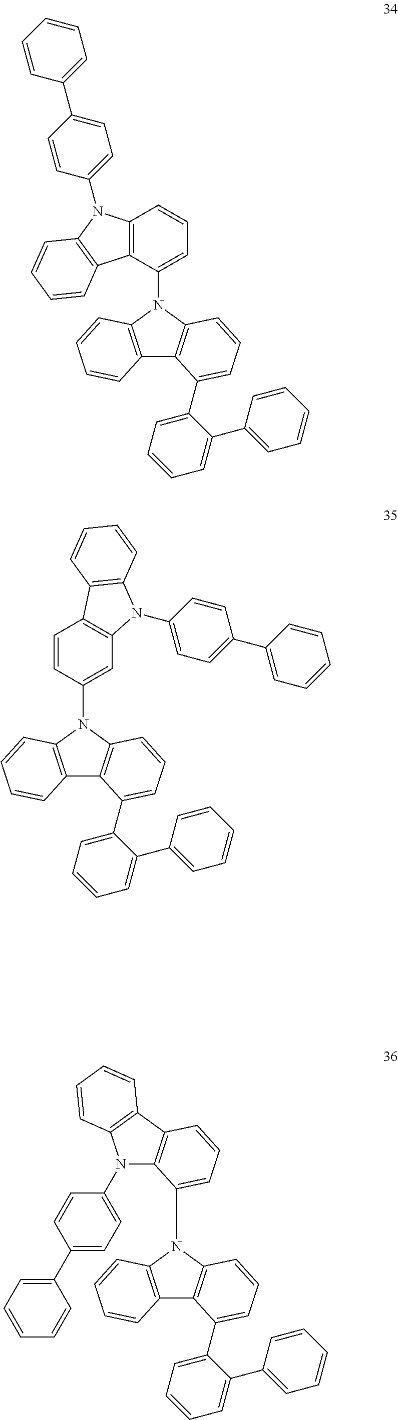 [SMILES: c1ccc(-c2ccc(-n3c4ccccc4c4c(-n5c6ccccc6c6c(-c7ccccc7-c7ccccc7)cccc65)cccc43)cc2)cc1 ; c1ccc(-c2ccc(-n3c4ccccc4c4ccc(-n5c6ccccc6c6c(-c7ccccc7-c7ccccc7)cccc65)cc43)cc2)cc1 ; c1ccc(-c2ccc(-n3c4ccccc4c4cccc(-n5c6ccccc6c6c(-c7ccccc7-c7ccccc7)cccc65)c43)cc2)cc1] 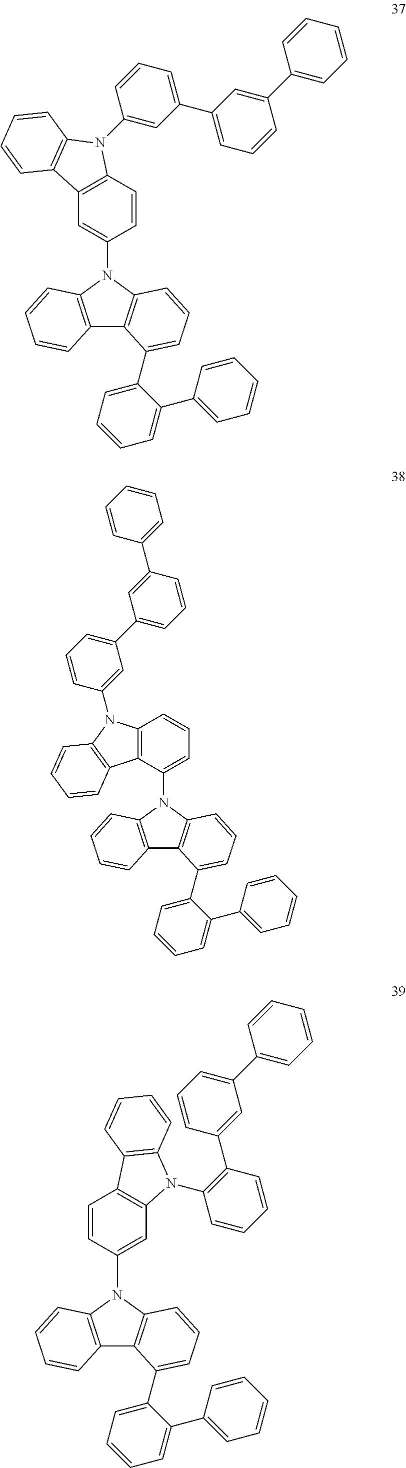 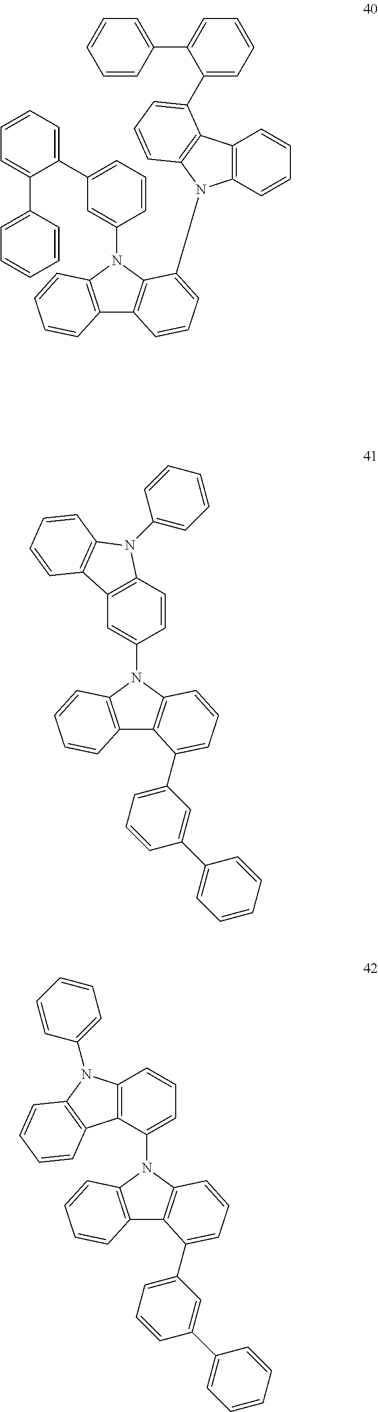 [SMILES: c1ccc(-c2ccccc2-c2cccc(-n3c4ccccc4c4cccc(-n5c6ccccc6c6c(-c7ccccc7-c7ccccc7)cccc65)c43)c2)cc1 ; c1ccc(-c2cccc(-c3cccc4c3c3ccccc3n4-c3ccc4c(c3)c3ccccc3n4-c3ccccc3)c2)cc1 ; c1ccc(-c2cccc(-c3cccc4c3c3ccccc3n4-c3cccc4c3c3ccccc3n4-c3ccccc3)c2)cc1] 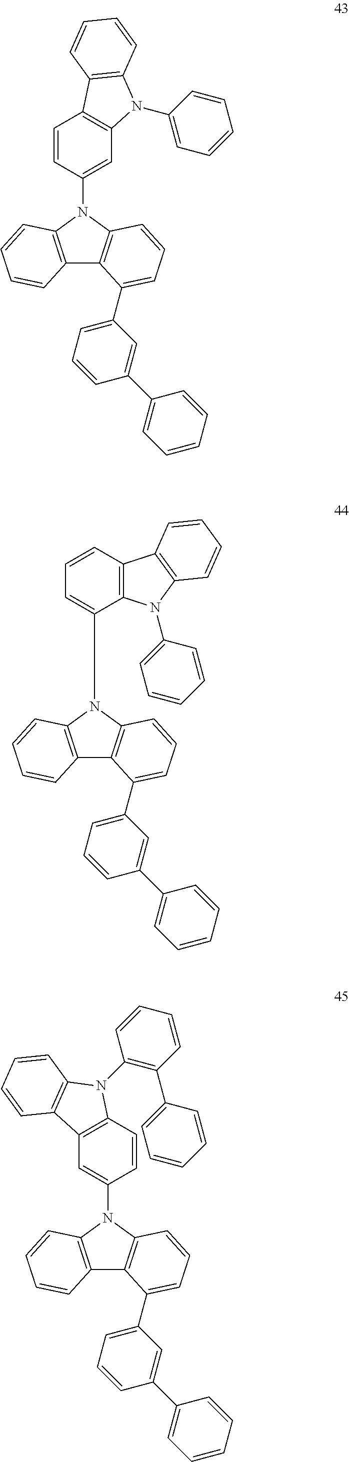 [SMILES: c1ccc(-c2cccc(-c3cccc4c3c3ccccc3n4-c3ccc4c5ccccc5n(-c5ccccc5)c4c3)c2)cc1 ; c1ccc(-c2cccc(-c3cccc4c3c3ccccc3n4-c3cccc4c5ccccc5n(-c5ccccc5)c34)c2)cc1 ; c1ccc(-c2cccc(-c3cccc4c3c3ccccc3n4-c3ccc4c(c3)c3ccccc3n4-c3ccccc3-c3ccccc3)c2)cc1] 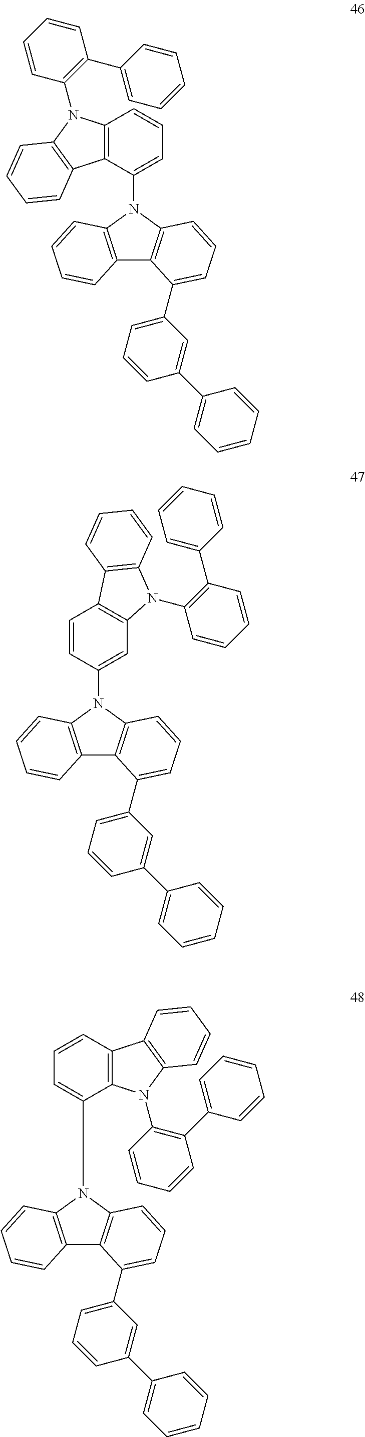 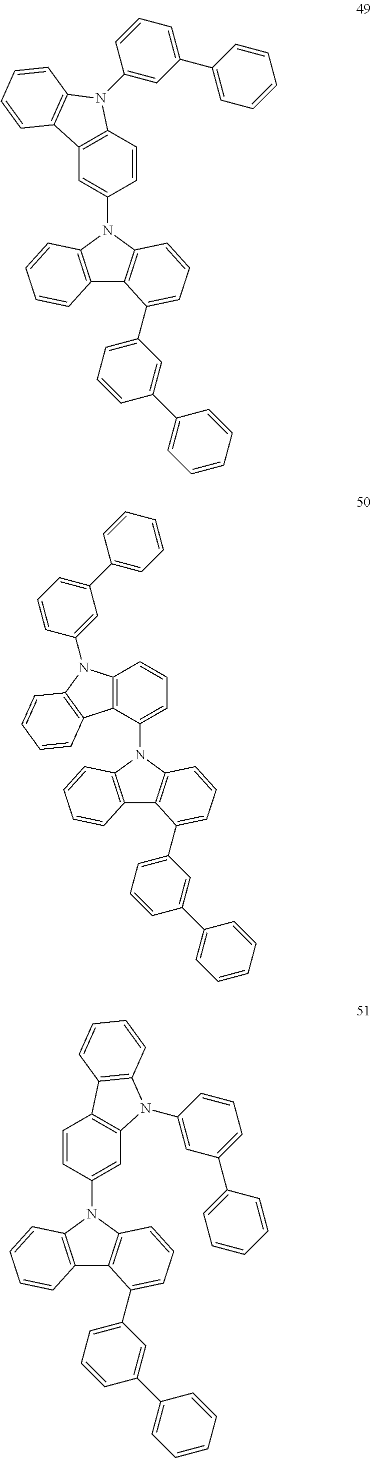 [SMILES: c1ccc(-c2cccc(-c3cccc4c3c3ccccc3n4-c3ccc4c(c3)c3ccccc3n4-c3cccc(-c4ccccc4)c3)c2)cc1 ; c1ccc(-c2cccc(-c3cccc4c3c3ccccc3n4-c3cccc4c3c3ccccc3n4-c3cccc(-c4ccccc4)c3)c2)cc1 ; c1ccc(-c2cccc(-c3cccc4c3c3ccccc3n4-c3ccc4c5ccccc5n(-c5cccc(-c6ccccc6)c5)c4c3)c2)cc1] 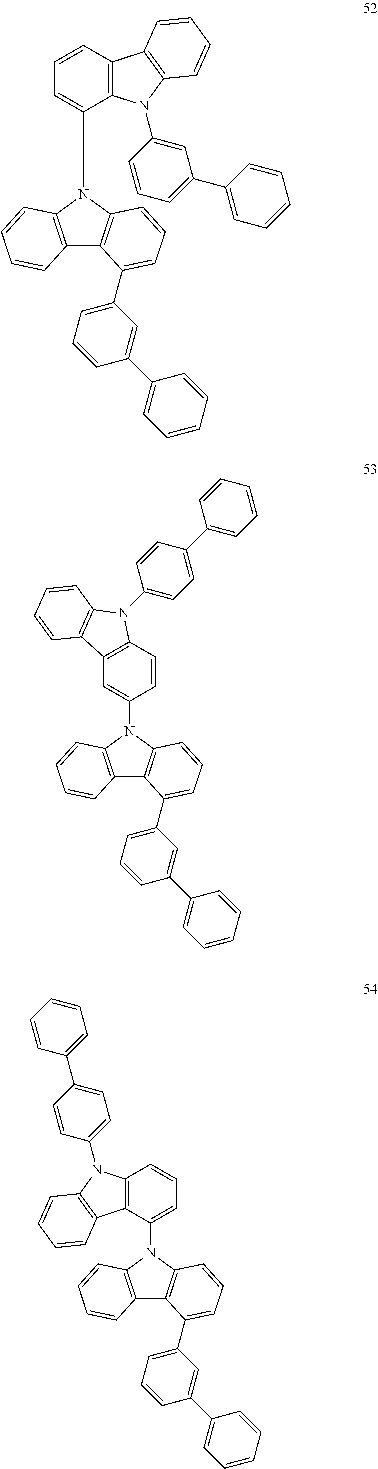 [SMILES: c1ccc(-c2cccc(-c3cccc4c3c3ccccc3n4-c3cccc4c5ccccc5n(-c5cccc(-c6ccccc6)c5)c34)c2)cc1 ; c1ccc(-c2ccc(-n3c4ccccc4c4cc(-n5c6ccccc6c6c(-c7cccc(-c8ccccc8)c7)cccc65)ccc43)cc2)cc1 ; c1ccc(-c2ccc(-n3c4ccccc4c4c(-n5c6ccccc6c6c(-c7cccc(-c8ccccc8)c7)cccc65)cccc43)cc2)cc1] 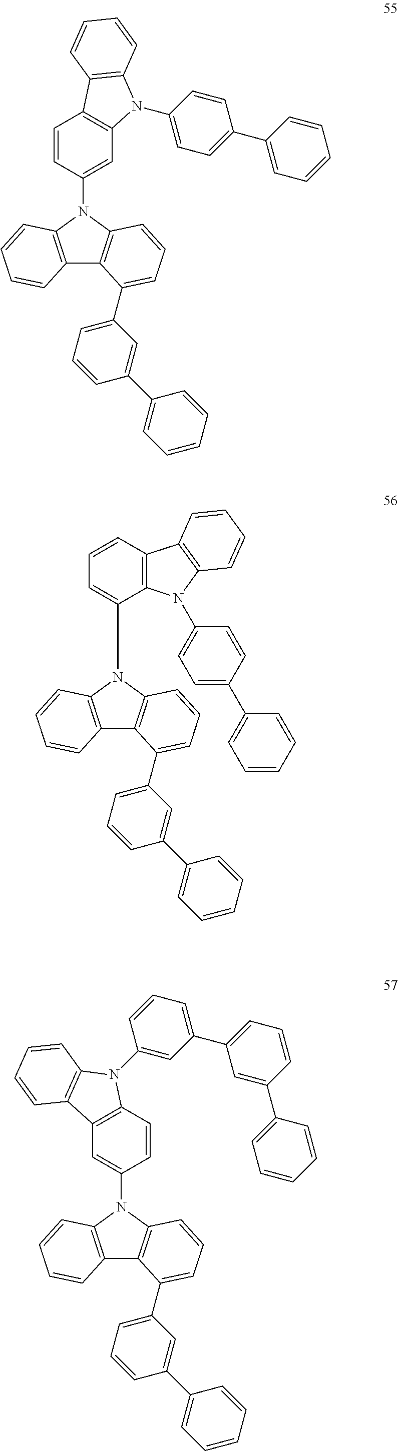 [SMILES: c1ccc(-c2ccc(-n3c4ccccc4c4ccc(-n5c6ccccc6c6c(-c7cccc(-c8ccccc8)c7)cccc65)cc43)cc2)cc1 ; c1ccc(-c2ccc(-n3c4ccccc4c4cccc(-n5c6ccccc6c6c(-c7cccc(-c8ccccc8)c7)cccc65)c43)cc2)cc1 ; c1ccc(-c2cccc(-c3cccc(-n4c5ccccc5c5cc(-n6c7ccccc7c7c(-c8cccc(-c9ccccc9)c8)cccc76)ccc54)c3)c2)cc1] 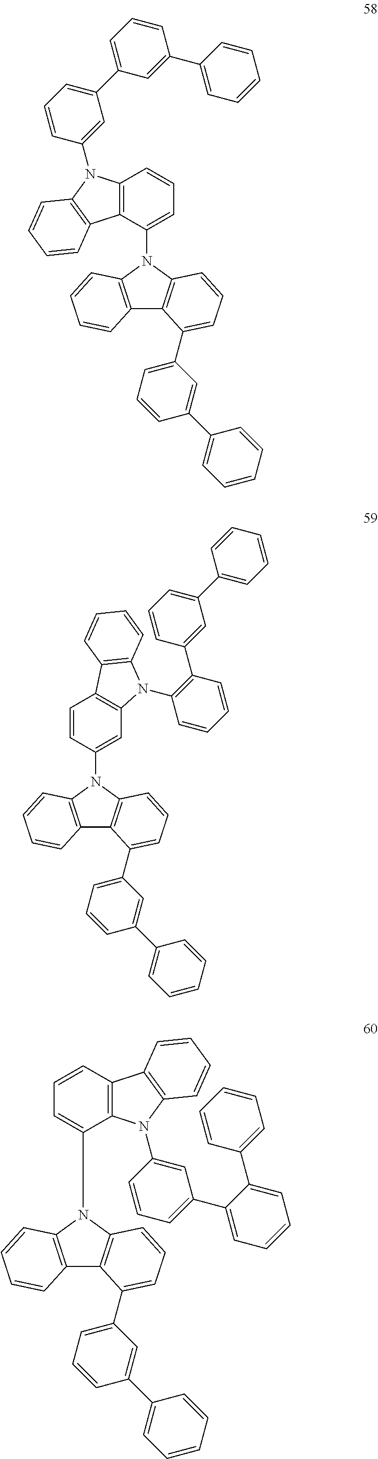 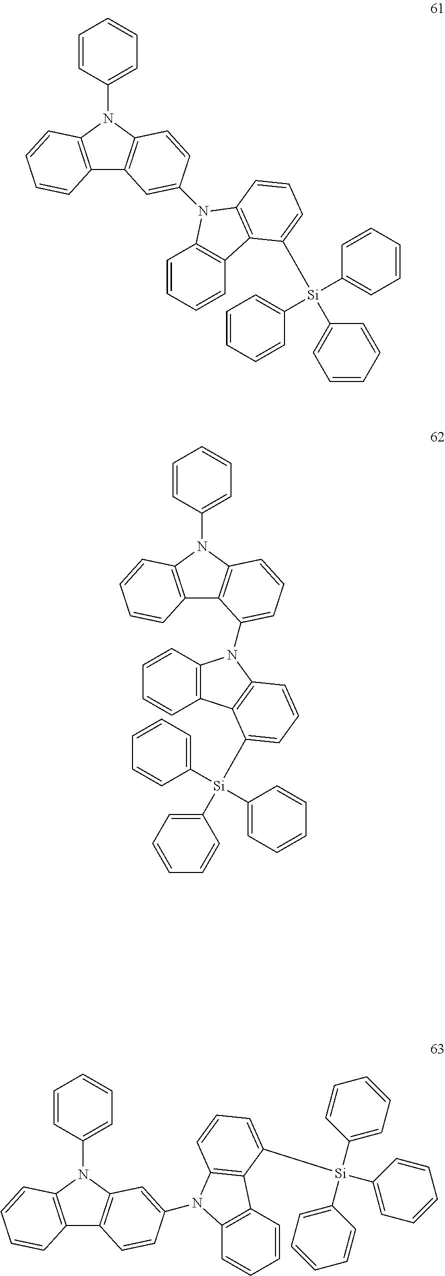 [SMILES: c1ccc(-n2c3ccccc3c3cc(-n4c5ccccc5c5c([Si](c6ccccc6)(c6ccccc6)c6ccccc6)cccc54)ccc32)cc1 ; c1ccc(-n2c3ccccc3c3c(-n4c5ccccc5c5c([Si](c6ccccc6)(c6ccccc6)c6ccccc6)cccc54)cccc32)cc1 ; c1ccc(-n2c3ccccc3c3ccc(-n4c5ccccc5c5c([Si](c6ccccc6)(c6ccccc6)c6ccccc6)cccc54)cc32)cc1] 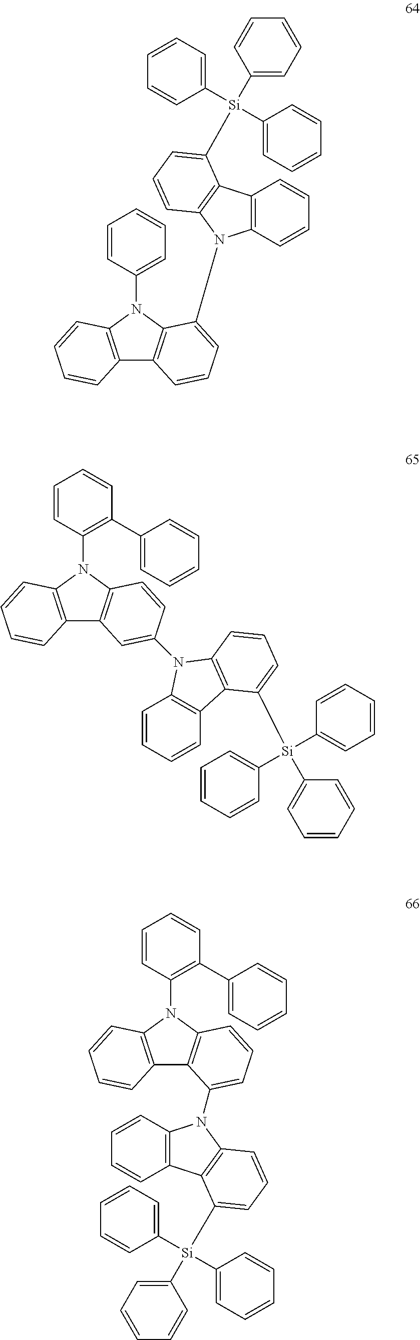 [SMILES: c1ccc(-n2c3ccccc3c3cccc(-n4c5ccccc5c5c([Si](c6ccccc6)(c6ccccc6)c6ccccc6)cccc54)c32)cc1 ; c1ccc(-c2ccccc2-n2c3ccccc3c3cc(-n4c5ccccc5c5c([Si](c6ccccc6)(c6ccccc6)c6ccccc6)cccc54)ccc32)cc1 ; c1ccc(-c2ccccc2-n2c3ccccc3c3c(-n4c5ccccc5c5c([Si](c6ccccc6)(c6ccccc6)c6ccccc6)cccc54)cccc32)cc1] 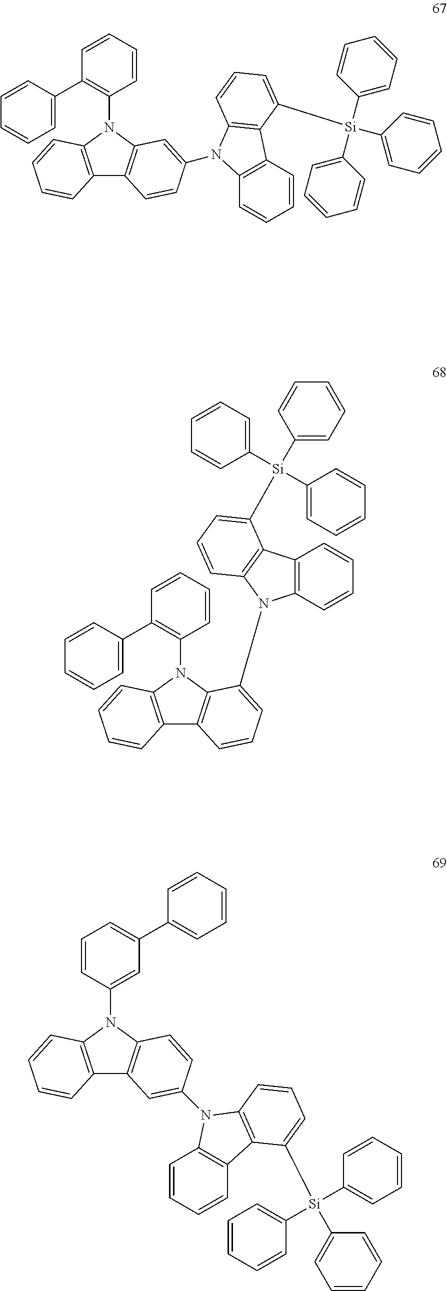 [SMILES: c1ccc(-c2ccccc2-n2c3ccccc3c3ccc(-n4c5ccccc5c5c([Si](c6ccccc6)(c6ccccc6)c6ccccc6)cccc54)cc32)cc1 ; c1ccc(-c2ccccc2-n2c3ccccc3c3cccc(-n4c5ccccc5c5c([Si](c6ccccc6)(c6ccccc6)c6ccccc6)cccc54)c32)cc1 ; c1ccc(-c2cccc(-n3c4ccccc4c4cc(-n5c6ccccc6c6c([Si](c7ccccc7)(c7ccccc7)c7ccccc7)cccc65)ccc43)c2)cc1] 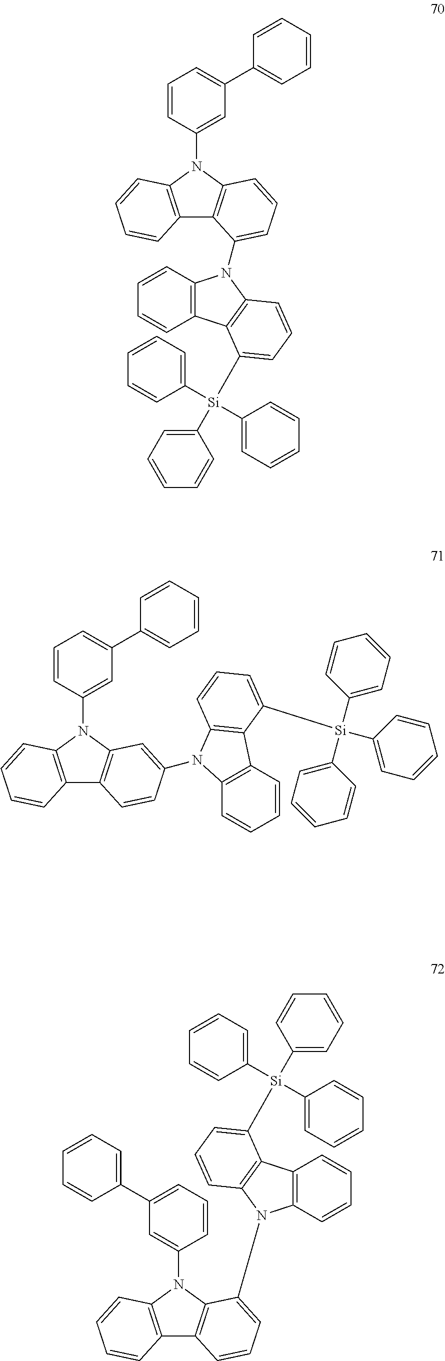 [SMILES: c1ccc(-c2cccc(-n3c4ccccc4c4c(-n5c6ccccc6c6c([Si](c7ccccc7)(c7ccccc7)c7ccccc7)cccc65)cccc43)c2)cc1 ; c1ccc(-c2cccc(-n3c4ccccc4c4ccc(-n5c6ccccc6c6c([Si](c7ccccc7)(c7ccccc7)c7ccccc7)cccc65)cc43)c2)cc1 ; c1ccc(-c2cccc(-n3c4ccccc4c4cccc(-n5c6ccccc6c6c([Si](c7ccccc7)(c7ccccc7)c7ccccc7)cccc65)c43)c2)cc1] 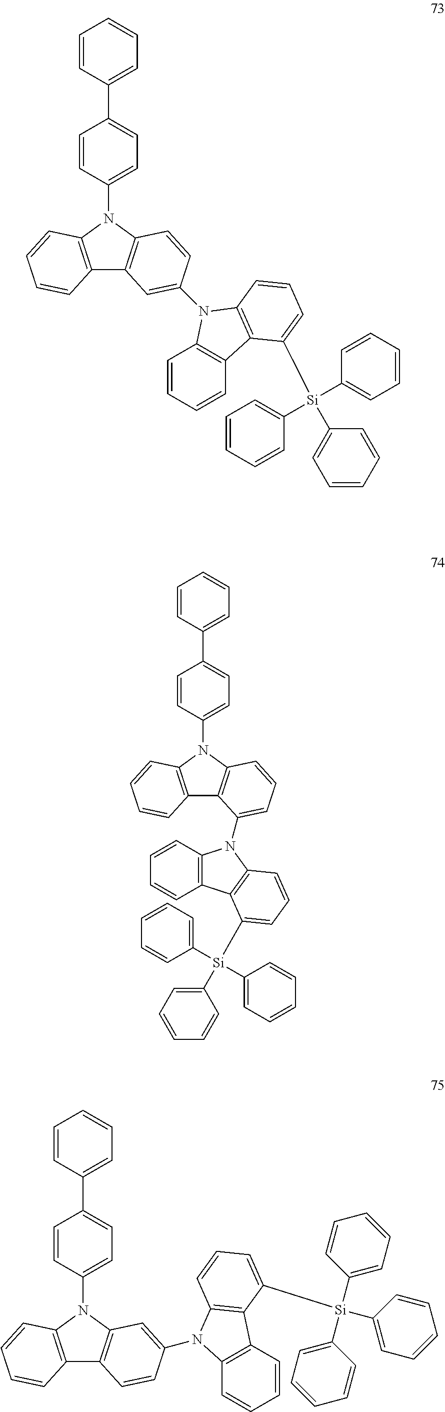 [SMILES: c1ccc(-c2ccc(-n3c4ccccc4c4cc(-n5c6ccccc6c6c([Si](c7ccccc7)(c7ccccc7)c7ccccc7)cccc65)ccc43)cc2)cc1 ; c1ccc(-c2ccc(-n3c4ccccc4c4c(-n5c6ccccc6c6c([Si](c7ccccc7)(c7ccccc7)c7ccccc7)cccc65)cccc43)cc2)cc1 ; c1ccc(-c2ccc(-n3c4ccccc4c4ccc(-n5c6ccccc6c6c([Si](c7ccccc7)(c7ccccc7)c7ccccc7)cccc65)cc43)cc2)cc1] 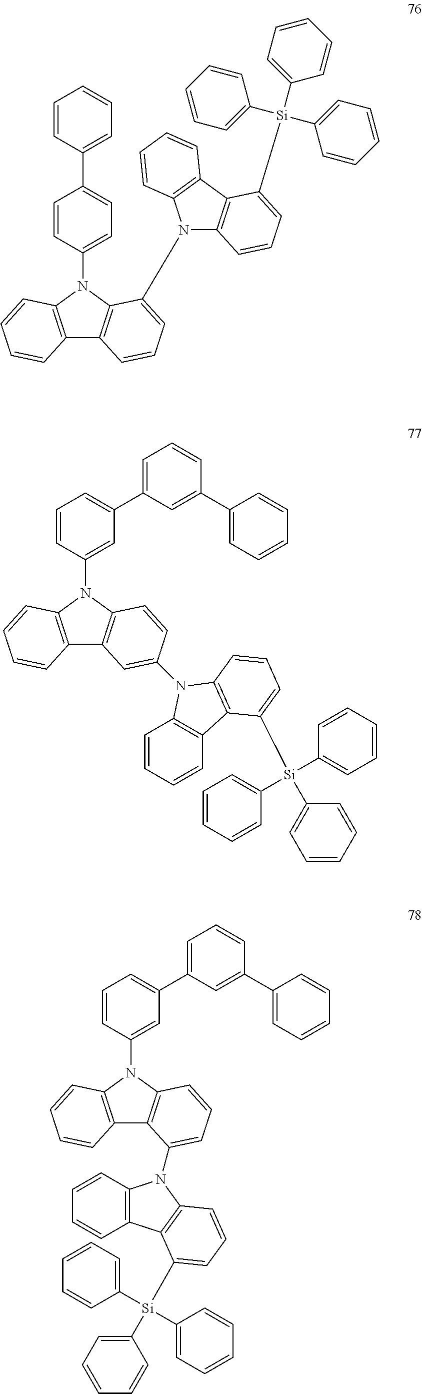 [SMILES: c1ccc(-c2ccc(-n3c4ccccc4c4cccc(-n5c6ccccc6c6c([Si](c7ccccc7)(c7ccccc7)c7ccccc7)cccc65)c43)cc2)cc1 ; c1ccc(-c2cccc(-c3cccc(-n4c5ccccc5c5cc(-n6c7ccccc7c7c([Si](c8ccccc8)(c8ccccc8)c8ccccc8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8ccccc8)cccc76)cccc54)c3)c2)cc1] 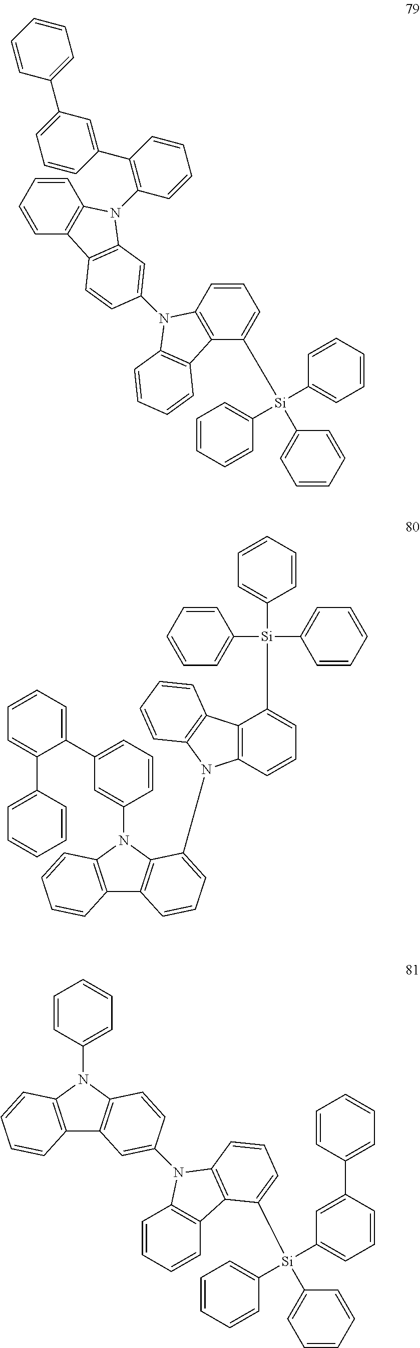 [SMILES: c1ccc(-c2cccc(-c3ccccc3-n3c4ccccc4c4ccc(-n5c6ccccc6c6c([Si](c7ccccc7)(c7ccccc7)c7ccccc7)cccc65)cc43)c2)cc1 ; c1ccc(-c2ccccc2-c2cccc(-n3c4ccccc4c4cccc(-n5c6ccccc6c6c([Si](c7ccccc7)(c7ccccc7)c7ccccc7)cccc65)c43)c2)cc1 ; c1ccc(-c2cccc([Si](c3ccccc3)(c3ccccc3)c3cccc4c3c3ccccc3n4-c3ccc4c(c3)c3ccccc3n4-c3ccccc3)c2)cc1] 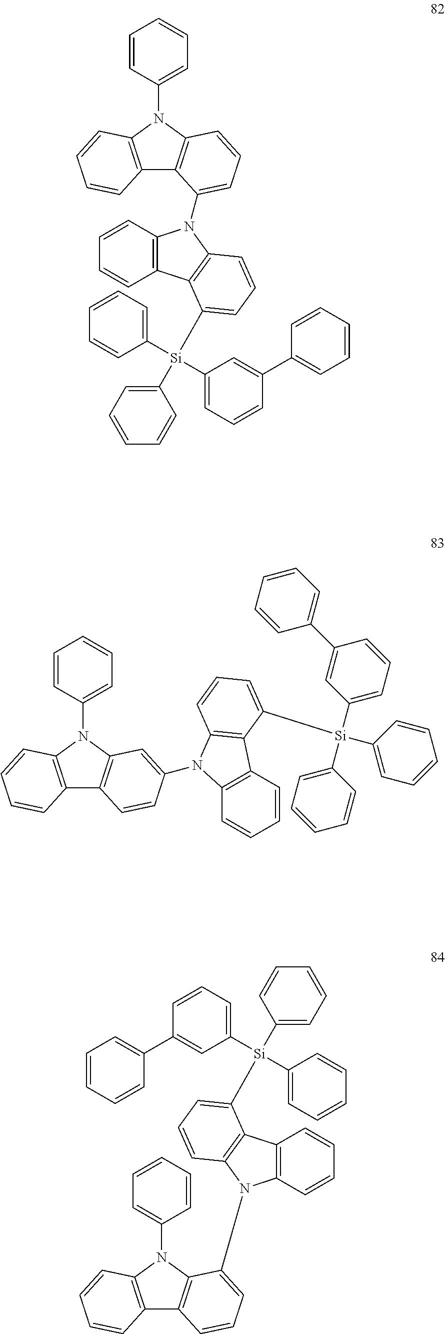 [SMILES: c1ccc(-c2cccc([Si](c3ccccc3)(c3ccccc3)c3cccc4c3c3ccccc3n4-c3cccc4c3c3ccccc3n4-c3ccccc3)c2)cc1 ; c1ccc(-c2cccc([Si](c3ccccc3)(c3ccccc3)c3cccc4c3c3ccccc3n4-c3ccc4c5ccccc5n(-c5ccccc5)c4c3)c2)cc1 ; c1ccc(-c2cccc([Si](c3ccccc3)(c3ccccc3)c3cccc4c3c3ccccc3n4-c3cccc4c5ccccc5n(-c5ccccc5)c34)c2)cc1] 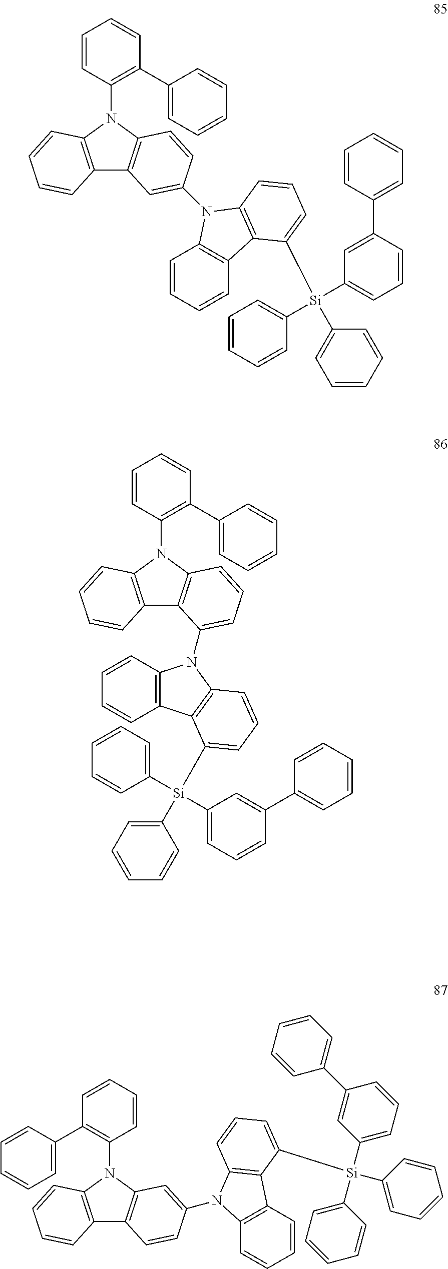 [SMILES: c1ccc(-c2cccc([Si](c3ccccc3)(c3ccccc3)c3cccc4c3c3ccccc3n4-c3ccc4c(c3)c3ccccc3n4-c3ccccc3-c3ccccc3)c2)cc1 ; c1ccc(-c2cccc([Si](c3ccccc3)(c3ccccc3)c3cccc4c3c3ccccc3n4-c3cccc4c3c3ccccc3n4-c3ccccc3-c3ccccc3)c2)cc1 ; c1ccc(-c2cccc([Si](c3ccccc3)(c3ccccc3)c3cccc4c3c3ccccc3n4-c3ccc4c5ccccc5n(-c5ccccc5-c5ccccc5)c4c3)c2)cc1] 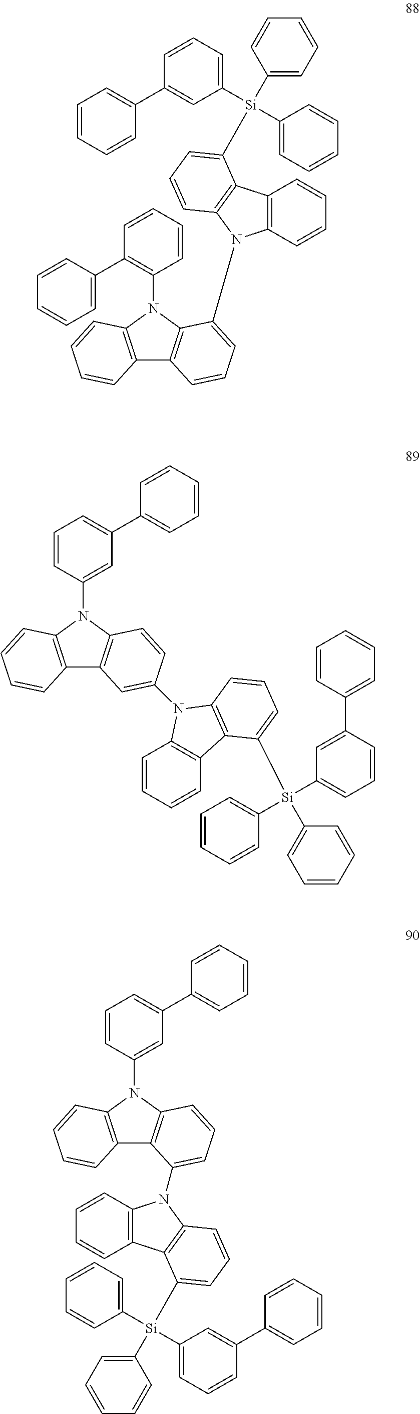 [SMILES: c1ccc(-c2cccc([Si](c3ccccc3)(c3ccccc3)c3cccc4c3c3ccccc3n4-c3cccc4c5ccccc5n(-c5ccccc5-c5ccccc5)c34)c2)cc1 ; c1ccc(-c2cccc(-n3c4ccccc4c4cc(-n5c6ccccc6c6c([Si](c7ccccc7)(c7ccccc7)c7cccc(-c8ccccc8)c7)cccc65)ccc43)c2)cc1 ; c1ccc(-c2cccc(-n3c4ccccc4c4c(-n5c6ccccc6c6c([Si](c7ccccc7)(c7ccccc7)c7cccc(-c8ccccc8)c7)cccc65)cccc43)c2)cc1] 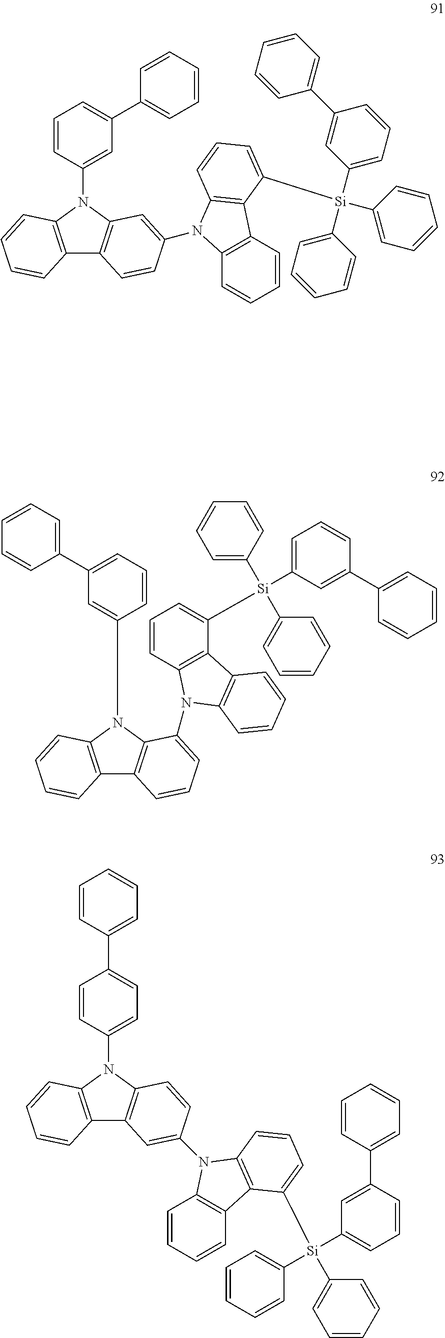 [SMILES: c1ccc(-c2cccc(-n3c4ccccc4c4ccc(-n5c6ccccc6c6c([Si](c7ccccc7)(c7ccccc7)c7cccc(-c8ccccc8)c7)cccc65)cc43)c2)cc1 ; c1ccc(-c2cccc(-n3c4ccccc4c4cccc(-n5c6ccccc6c6c([Si](c7ccccc7)(c7ccccc7)c7cccc(-c8ccccc8)c7)cccc65)c43)c2)cc1 ; c1ccc(-c2ccc(-n3c4ccccc4c4cc(-n5c6ccccc6c6c([Si](c7ccccc7)(c7ccccc7)c7cccc(-c8ccccc8)c7)cccc65)ccc43)cc2)cc1] 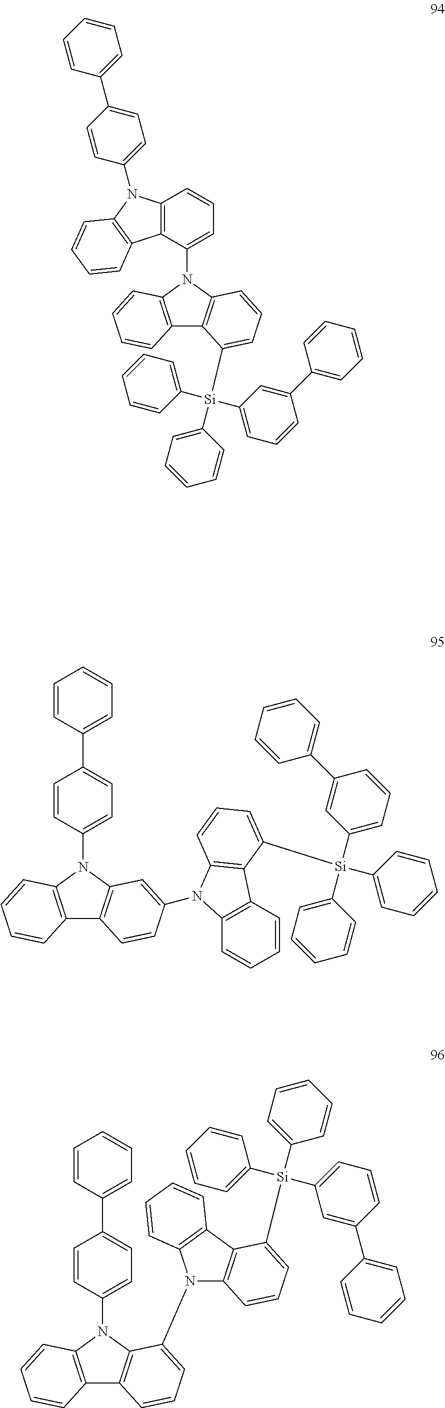 [SMILES: c1ccc(-c2ccc(-n3c4ccccc4c4c(-n5c6ccccc6c6c([Si](c7ccccc7)(c7ccccc7)c7cccc(-c8ccccc8)c7)cccc65)cccc43)cc2)cc1 ; c1ccc(-c2ccc(-n3c4ccccc4c4ccc(-n5c6ccccc6c6c([Si](c7ccccc7)(c7ccccc7)c7cccc(-c8ccccc8)c7)cccc65)cc43)cc2)cc1 ; c1ccc(-c2ccc(-n3c4ccccc4c4cccc(-n5c6ccccc6c6c([Si](c7ccccc7)(c7ccccc7)c7cccc(-c8ccccc8)c7)cccc65)c43)cc2)cc1] 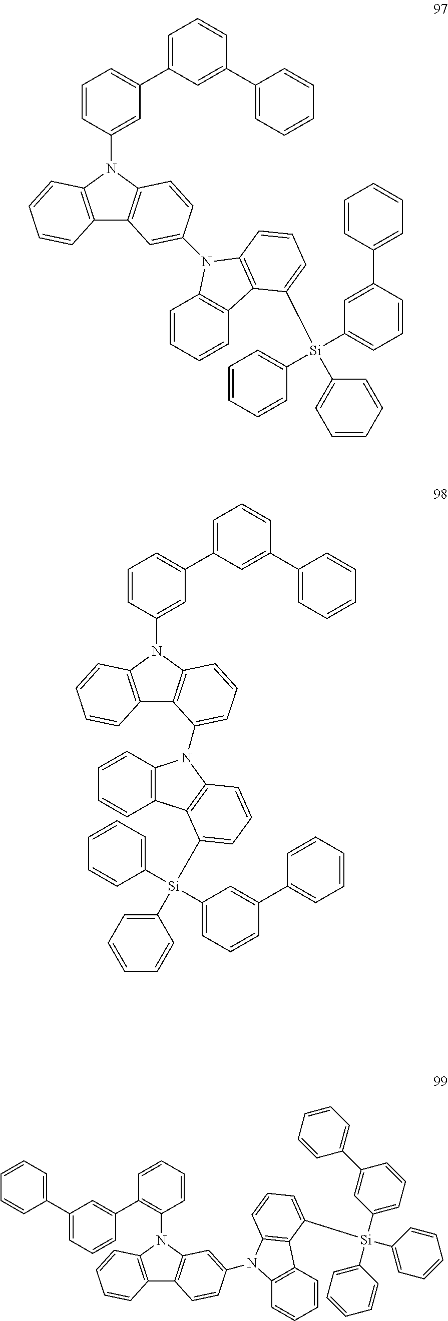 [SMILES: c1ccc(-c2cccc(-c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8cccc(-c9ccccc9)c8)cccc76)cccc54)c3)c2)cc1 ; c1ccc(-c2cccc(-c3ccccc3-n3c4ccccc4c4ccc(-n5c6ccccc6c6c([Si](c7ccccc7)(c7ccccc7)c7cccc(-c8ccccc8)c7)cccc65)cc43)c2)cc1] 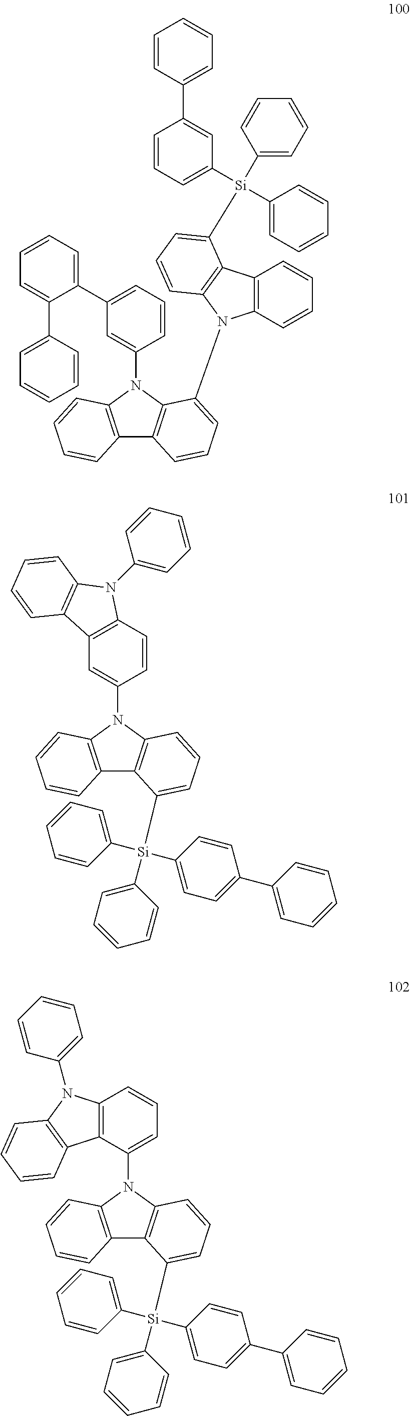 [SMILES: c1ccc(-c2cccc([Si](c3ccccc3)(c3ccccc3)c3cccc4c3c3ccccc3n4-c3cccc4c5ccccc5n(-c5cccc(-c6ccccc6-c6ccccc6)c5)c34)c2)cc1 ; c1ccc(-c2ccc([Si](c3ccccc3)(c3ccccc3)c3cccc4c3c3ccccc3n4-c3ccc4c(c3)c3ccccc3n4-c3ccccc3)cc2)cc1 ; c1ccc(-c2ccc([Si](c3ccccc3)(c3ccccc3)c3cccc4c3c3ccccc3n4-c3cccc4c3c3ccccc3n4-c3ccccc3)cc2)cc1] 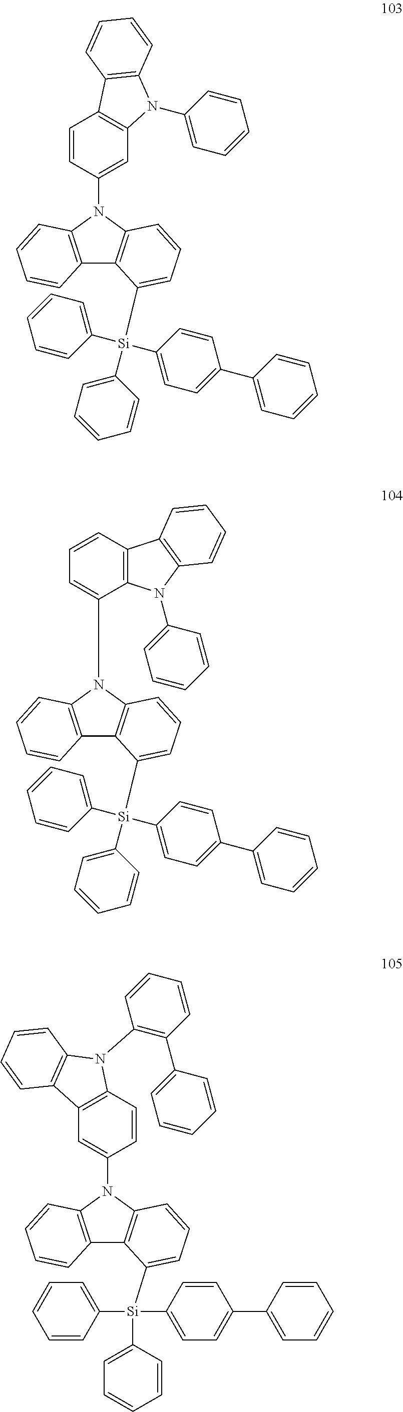 [SMILES: c1ccc(-c2ccc([Si](c3ccccc3)(c3ccccc3)c3cccc4c3c3ccccc3n4-c3ccc4c5ccccc5n(-c5ccccc5)c4c3)cc2)cc1 ; c1ccc(-c2ccc([Si](c3ccccc3)(c3ccccc3)c3cccc4c3c3ccccc3n4-c3cccc4c5ccccc5n(-c5ccccc5)c34)cc2)cc1 ; c1ccc(-c2ccc([Si](c3ccccc3)(c3ccccc3)c3cccc4c3c3ccccc3n4-c3ccc4c(c3)c3ccccc3n4-c3ccccc3-c3ccccc3)cc2)cc1] 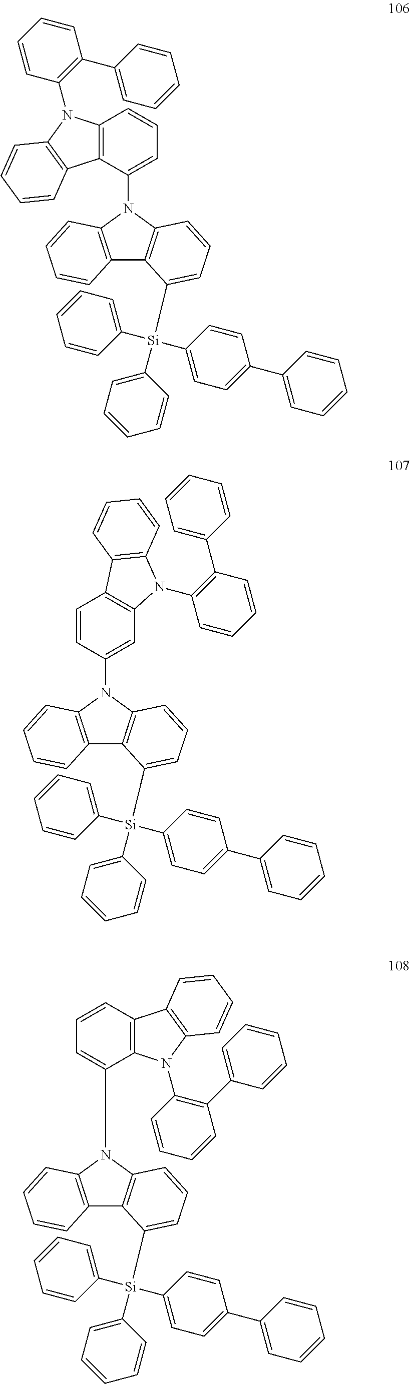 [SMILES: c1ccc(-c2ccc([Si](c3ccccc3)(c3ccccc3)c3cccc4c3c3ccccc3n4-c3cccc4c3c3ccccc3n4-c3ccccc3-c3ccccc3)cc2)cc1 ; c1ccc(-c2ccc([Si](c3ccccc3)(c3ccccc3)c3cccc4c3c3ccccc3n4-c3ccc4c5ccccc5n(-c5ccccc5-c5ccccc5)c4c3)cc2)cc1 ; c1ccc(-c2ccc([Si](c3ccccc3)(c3ccccc3)c3cccc4c3c3ccccc3n4-c3cccc4c5ccccc5n(-c5ccccc5-c5ccccc5)c34)cc2)cc1] 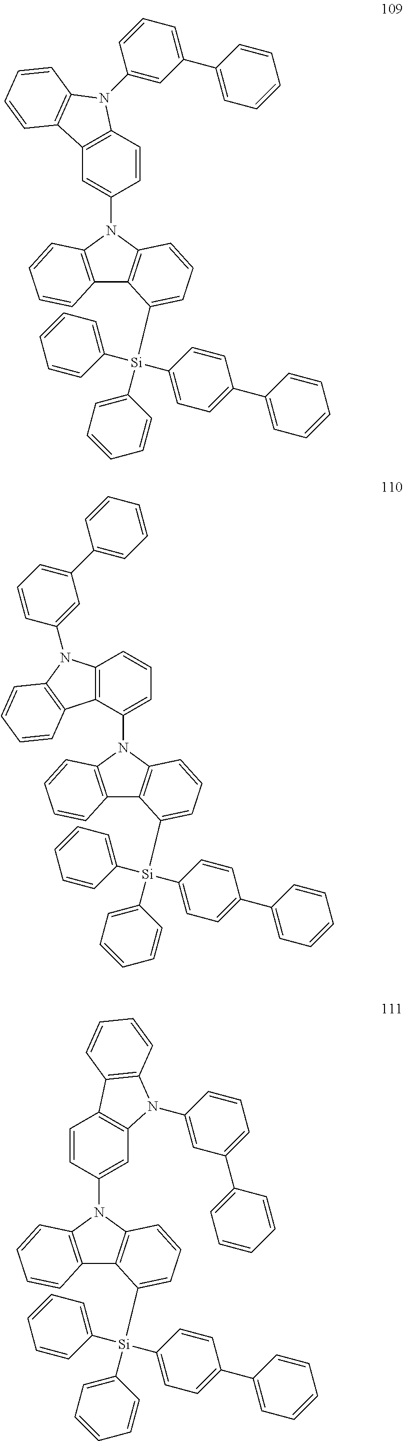 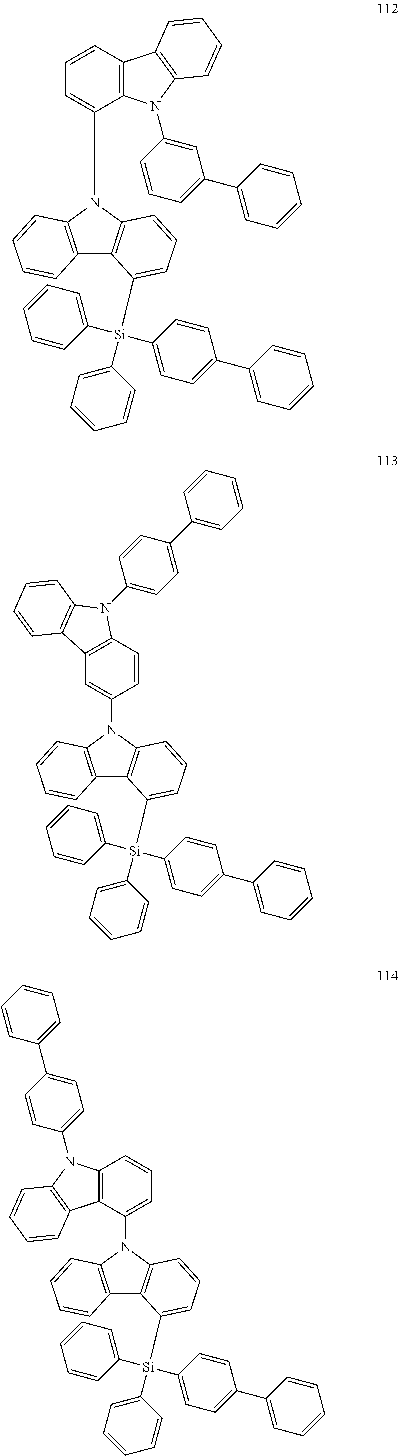 [SMILES: c1ccc(-c2ccc([Si](c3ccccc3)(c3ccccc3)c3cccc4c3c3ccccc3n4-c3cccc4c5ccccc5n(-c5cccc(-c6ccccc6)c5)c34)cc2)cc1 ; c1ccc(-c2ccc(-n3c4ccccc4c4cc(-n5c6ccccc6c6c([Si](c7ccccc7)(c7ccccc7)c7ccc(-c8ccccc8)cc7)cccc65)ccc43)cc2)cc1 ; c1ccc(-c2ccc(-n3c4ccccc4c4c(-n5c6ccccc6c6c([Si](c7ccccc7)(c7ccccc7)c7ccc(-c8ccccc8)cc7)cccc65)cccc43)cc2)cc1] 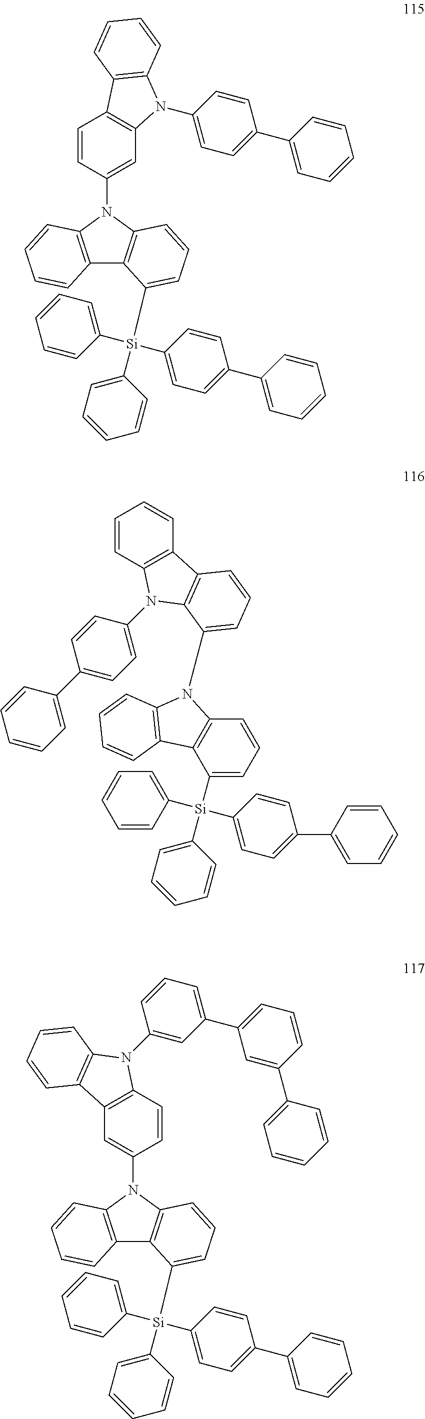 [SMILES: c1ccc(-c2ccc(-n3c4ccccc4c4ccc(-n5c6ccccc6c6c([Si](c7ccccc7)(c7ccccc7)c7ccc(-c8ccccc8)cc7)cccc65)cc43)cc2)cc1 ; c1ccc(-c2ccc(-n3c4ccccc4c4cccc(-n5c6ccccc6c6c([Si](c7ccccc7)(c7ccccc7)c7ccc(-c8ccccc8)cc7)cccc65)c43)cc2)cc1 ; c1ccc(-c2ccc([Si](c3ccccc3)(c3ccccc3)c3cccc4c3c3ccccc3n4-c3ccc4c(c3)c3ccccc3n4-c3cccc(-c4cccc(-c5ccccc5)c4)c3)cc2)cc1] 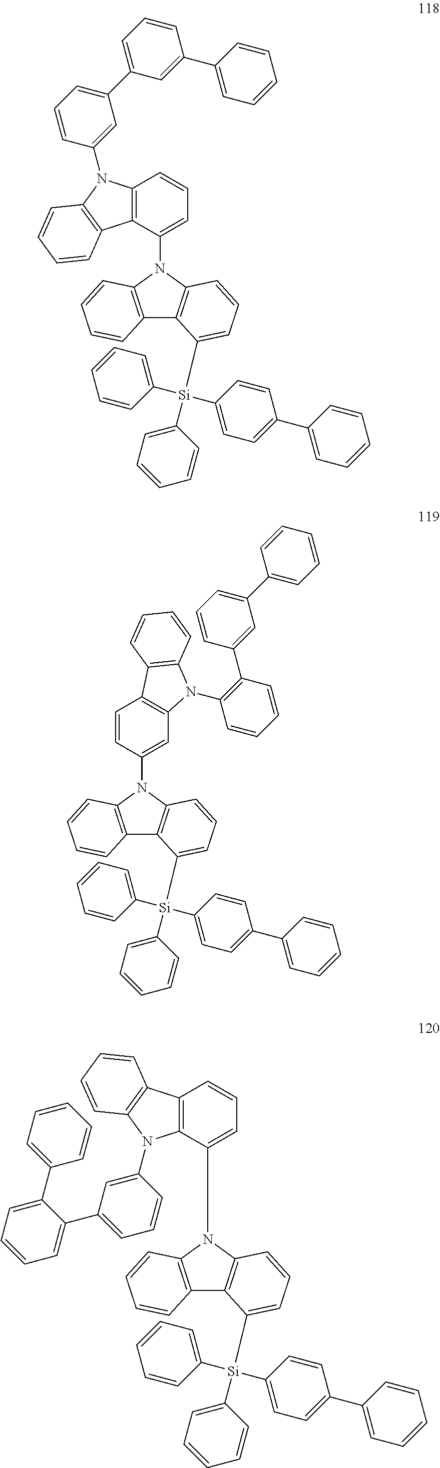 [SMILES: c1ccc(-c2ccc([Si](c3ccccc3)(c3ccccc3)c3cccc4c3c3ccccc3n4-c3cccc4c3c3ccccc3n4-c3cccc(-c4cccc(-c5ccccc5)c4)c3)cc2)cc1 ; c1ccc(-c2ccc([Si](c3ccccc3)(c3ccccc3)c3cccc4c3c3ccccc3n4-c3ccc4c5ccccc5n(-c5ccccc5-c5cccc(-c6ccccc6)c5)c4c3)cc2)cc1 ; c1ccc(-c2ccc([Si](c3ccccc3)(c3ccccc3)c3cccc4c3c3ccccc3n4-c3cccc4c5ccccc5n(-c5cccc(-c6ccccc6-c6ccccc6)c5)c34)cc2)cc1] 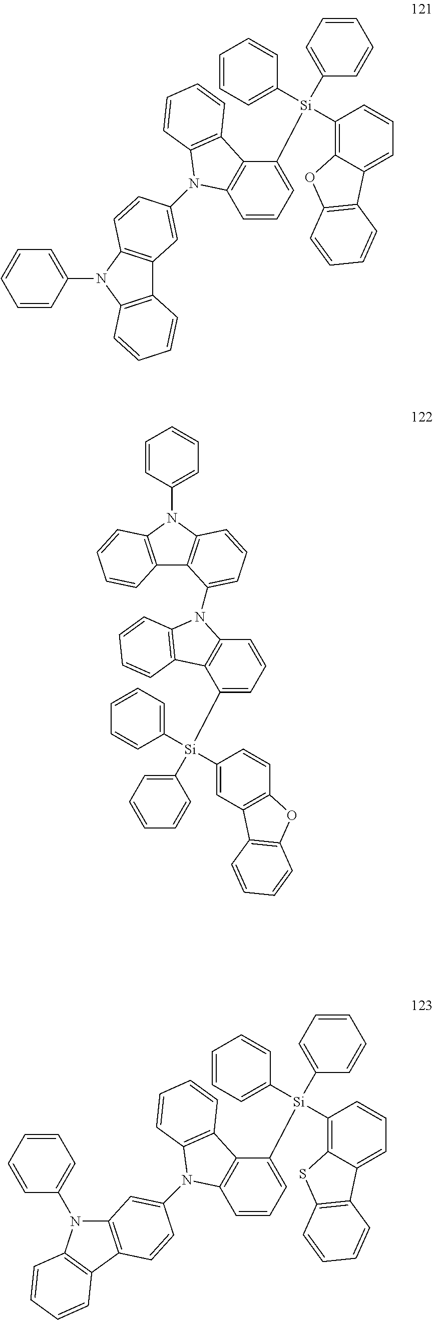 [SMILES: c1ccc(-n2c3ccccc3c3cc(-n4c5ccccc5c5c([Si](c6ccccc6)(c6ccccc6)c6cccc7c6oc6ccccc67)cccc54)ccc32)cc1 ; c1ccc(-n2c3ccccc3c3c(-n4c5ccccc5c5c([Si](c6ccccc6)(c6ccccc6)c6ccc7oc8ccccc8c7c6)cccc54)cccc32)cc1 ; c1ccc(-n2c3ccccc3c3ccc(-n4c5ccccc5c5c([Si](c6ccccc6)(c6ccccc6)c6cccc7c6sc6ccccc67)cccc54)cc32)cc1] 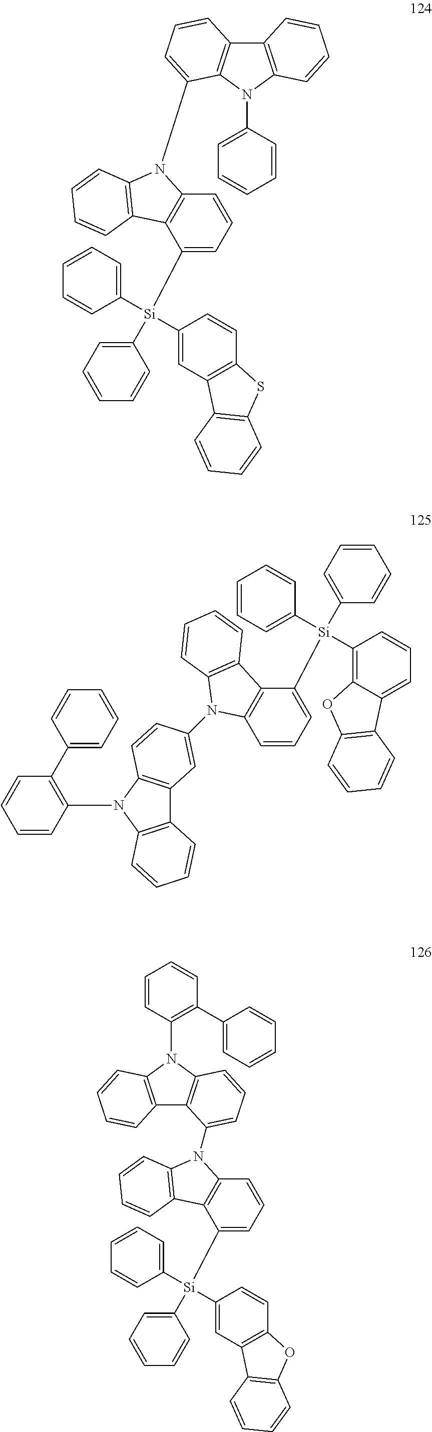 [SMILES: c1ccc(-n2c3ccccc3c3cccc(-n4c5ccccc5c5c([Si](c6ccccc6)(c6ccccc6)c6ccc7sc8ccccc8c7c6)cccc54)c32)cc1 ; c1ccc(-c2ccccc2-n2c3ccccc3c3cc(-n4c5ccccc5c5c([Si](c6ccccc6)(c6ccccc6)c6cccc7c6oc6ccccc67)cccc54)ccc32)cc1 ; c1ccc(-c2ccccc2-n2c3ccccc3c3c(-n4c5ccccc5c5c([Si](c6ccccc6)(c6ccccc6)c6ccc7oc8ccccc8c7c6)cccc54)cccc32)cc1] 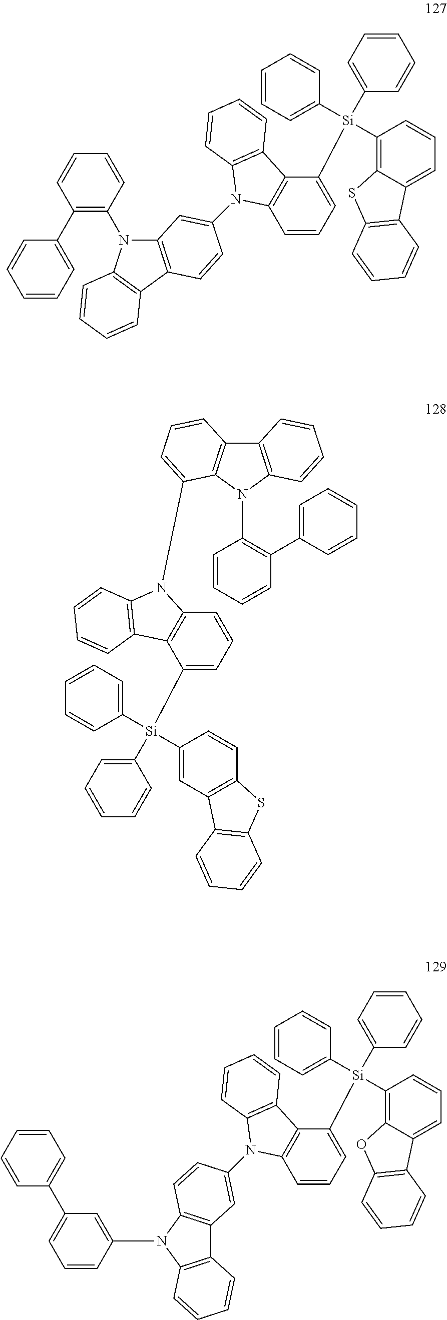 [SMILES: c1ccc(-c2ccccc2-n2c3ccccc3c3ccc(-n4c5ccccc5c5c([Si](c6ccccc6)(c6ccccc6)c6cccc7c6sc6ccccc67)cccc54)cc32)cc1 ; c1ccc(-c2ccccc2-n2c3ccccc3c3cccc(-n4c5ccccc5c5c([Si](c6ccccc6)(c6ccccc6)c6ccc7sc8ccccc8c7c6)cccc54)c32)cc1 ; c1ccc(-c2cccc(-n3c4ccccc4c4cc(-n5c6ccccc6c6c([Si](c7ccccc7)(c7ccccc7)c7cccc8c7oc7ccccc78)cccc65)ccc43)c2)cc1] 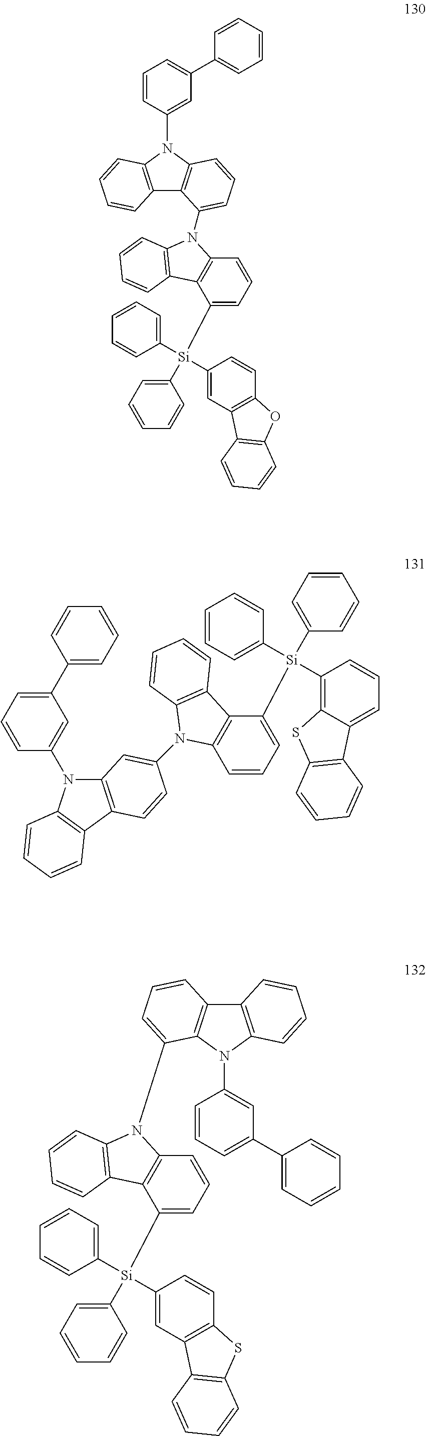 [SMILES: c1ccc(-c2cccc(-n3c4ccccc4c4c(-n5c6ccccc6c6c([Si](c7ccccc7)(c7ccccc7)c7ccc8oc9ccccc9c8c7)cccc65)cccc43)c2)cc1 ; c1ccc(-c2cccc(-n3c4ccccc4c4ccc(-n5c6ccccc6c6c([Si](c7ccccc7)(c7ccccc7)c7cccc8c7sc7ccccc78)cccc65)cc43)c2)cc1 ; c1ccc(-c2cccc(-n3c4ccccc4c4cccc(-n5c6ccccc6c6c([Si](c7ccccc7)(c7ccccc7)c7ccc8sc9ccccc9c8c7)cccc65)c43)c2)cc1] 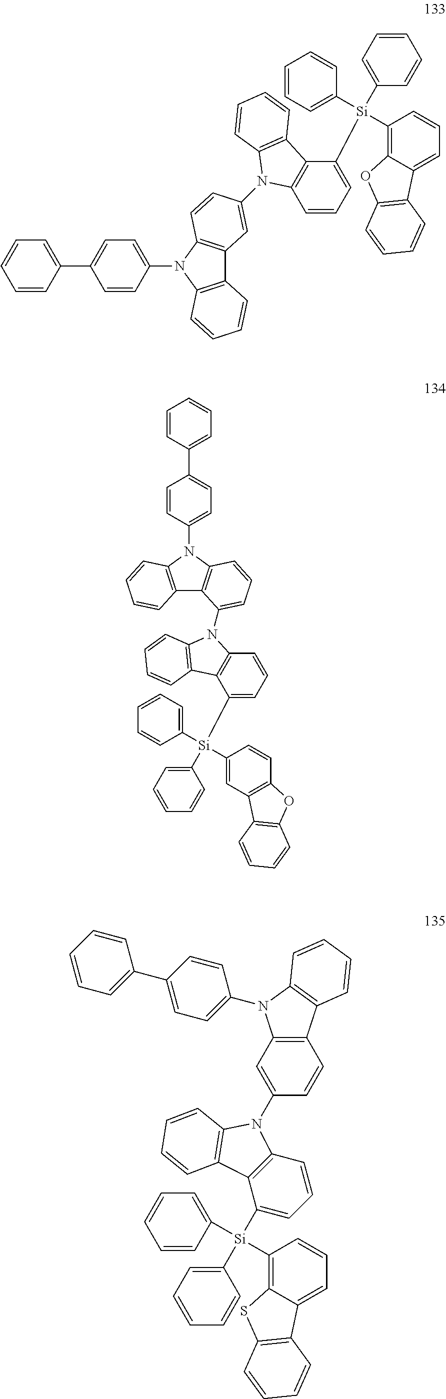 [SMILES: c1ccc(-c2ccc(-n3c4ccccc4c4cc(-n5c6ccccc6c6c([Si](c7ccccc7)(c7ccccc7)c7cccc8c7oc7ccccc78)cccc65)ccc43)cc2)cc1 ; c1ccc(-c2ccc(-n3c4ccccc4c4c(-n5c6ccccc6c6c([Si](c7ccccc7)(c7ccccc7)c7ccc8oc9ccccc9c8c7)cccc65)cccc43)cc2)cc1 ; c1ccc(-c2ccc(-n3c4ccccc4c4ccc(-n5c6ccccc6c6c([Si](c7ccccc7)(c7ccccc7)c7cccc8c7sc7ccccc78)cccc65)cc43)cc2)cc1] 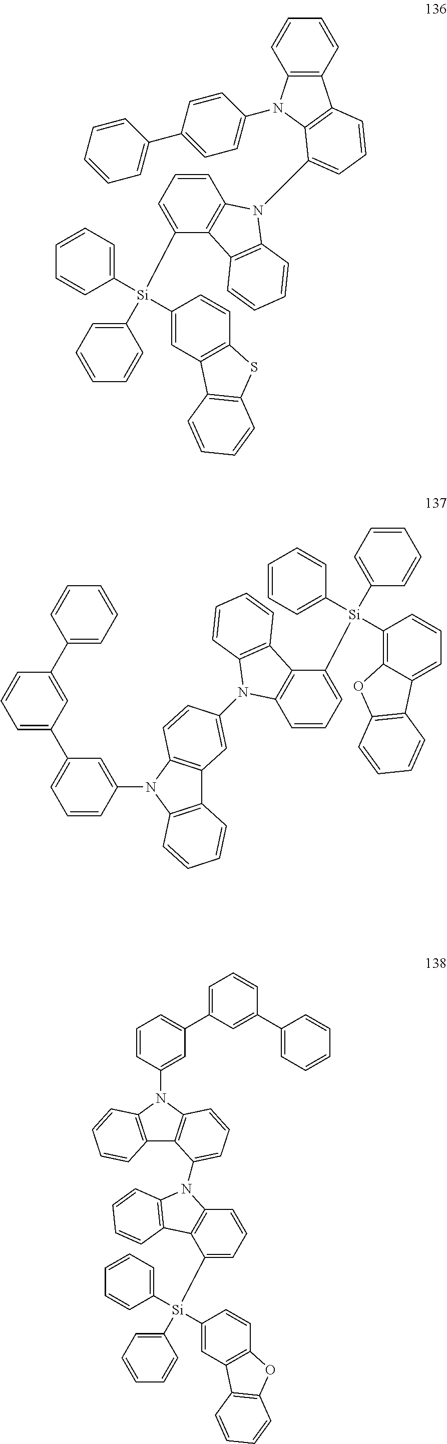 [SMILES: c1ccc(-c2ccc(-n3c4ccccc4c4cccc(-n5c6ccccc6c6c([Si](c7ccccc7)(c7ccccc7)c7ccc8sc9ccccc9c8c7)cccc65)c43)cc2)cc1 ; c1ccc(-c2cccc(-c3cccc(-n4c5ccccc5c5cc(-n6c7ccccc7c7c([Si](c8ccccc8)(c8ccccc8)c8cccc9c8oc8ccccc89)cccc76)ccc54)c3)c2)cc1 ; c1ccc(-c2cccc(-c3cccc(-n4c5ccccc5c5c(-n6c7ccccc7c7c([Si](c8ccccc8)(c8ccccc8)c8ccc9oc%10ccccc%10c9c8)cccc76)cccc54)c3)c2)cc1] 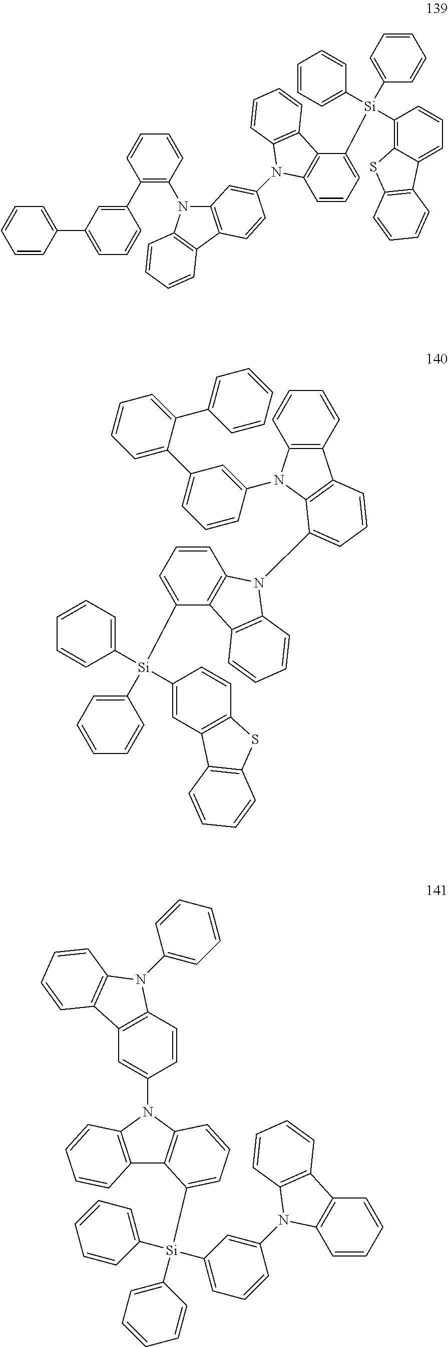 [SMILES: c1ccc(-c2cccc(-c3ccccc3-n3c4ccccc4c4ccc(-n5c6ccccc6c6c([Si](c7ccccc7)(c7ccccc7)c7cccc8c7sc7ccccc78)cccc65)cc43)c2)cc1 ; c1ccc(-c2ccccc2-c2cccc(-n3c4ccccc4c4cccc(-n5c6ccccc6c6c([Si](c7ccccc7)(c7ccccc7)c7ccc8sc9ccccc9c8c7)cccc65)c43)c2)cc1 ; c1ccc(-n2c3ccccc3c3cc(-n4c5ccccc5c5c([Si](c6ccccc6)(c6ccccc6)c6cccc(-n7c8ccccc8c8ccccc87)c6)cccc54)ccc32)cc1] 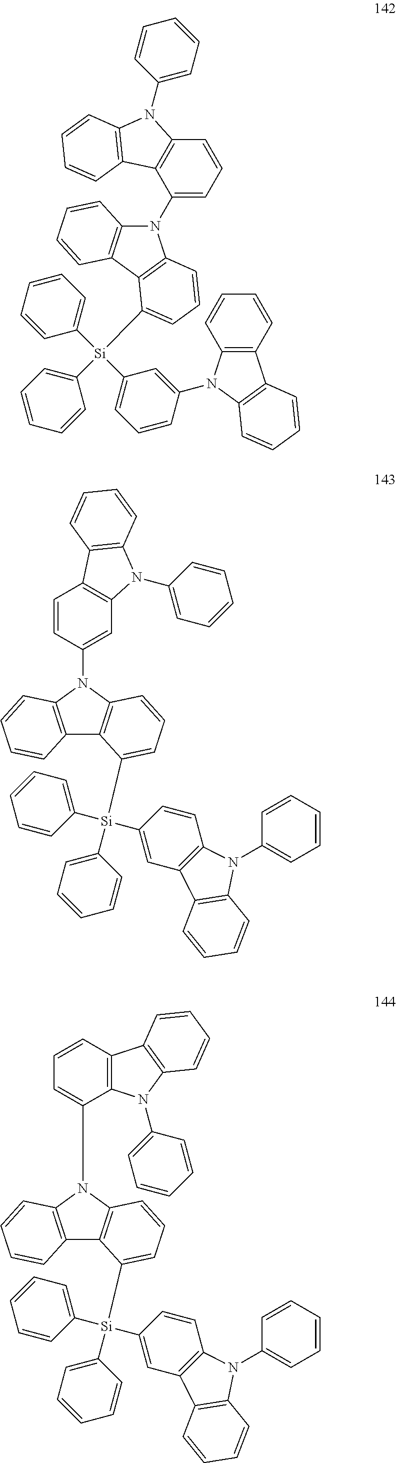 [SMILES: c1ccc(-n2c3ccccc3c3c(-n4c5ccccc5c5c([Si](c6ccccc6)(c6ccccc6)c6cccc(-n7c8ccccc8c8ccccc87)c6)cccc54)cccc32)cc1 ; c1ccc(-n2c3ccccc3c3cc([Si](c4ccccc4)(c4ccccc4)c4cccc5c4c4ccccc4n5-c4ccc5c6ccccc6n(-c6ccccc6)c5c4)ccc32)cc1 ; c1ccc(-n2c3ccccc3c3cc([Si](c4ccccc4)(c4ccccc4)c4cccc5c4c4ccccc4n5-c4cccc5c6ccccc6n(-c6ccccc6)c45)ccc32)cc1] 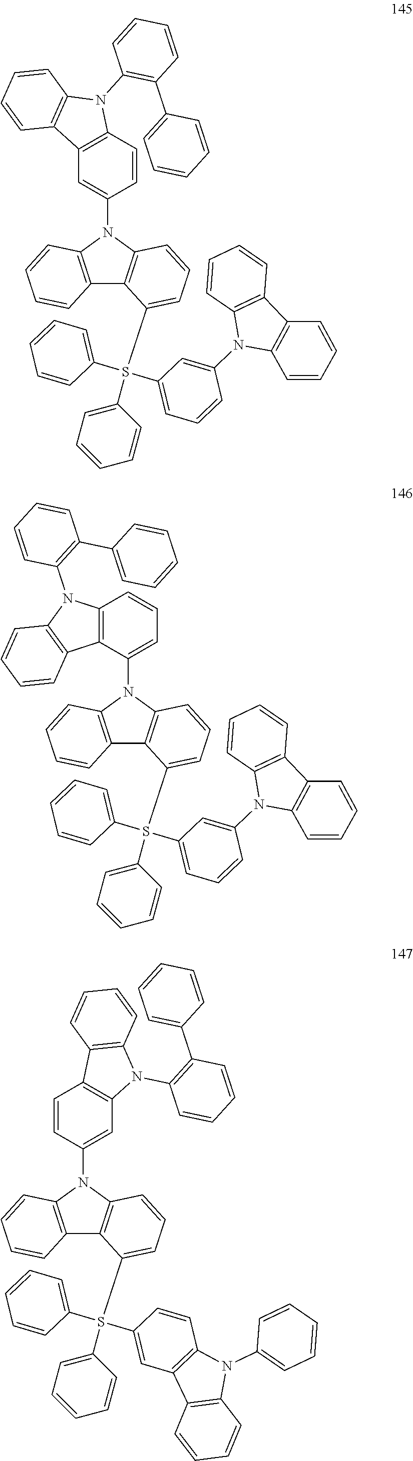 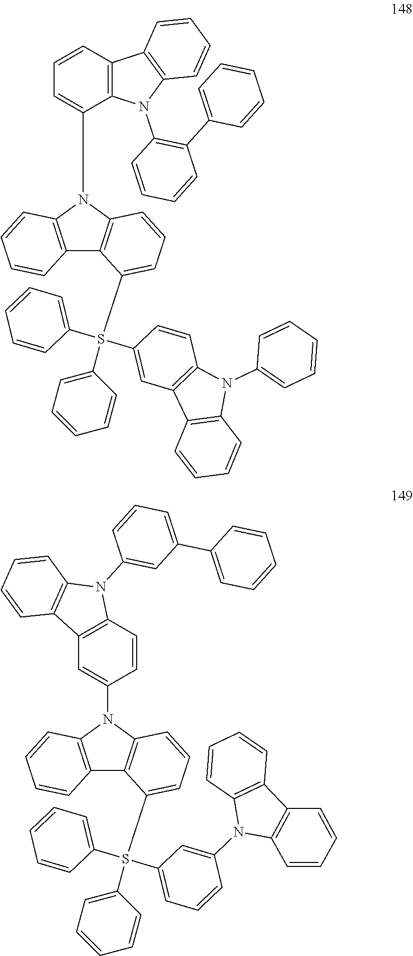 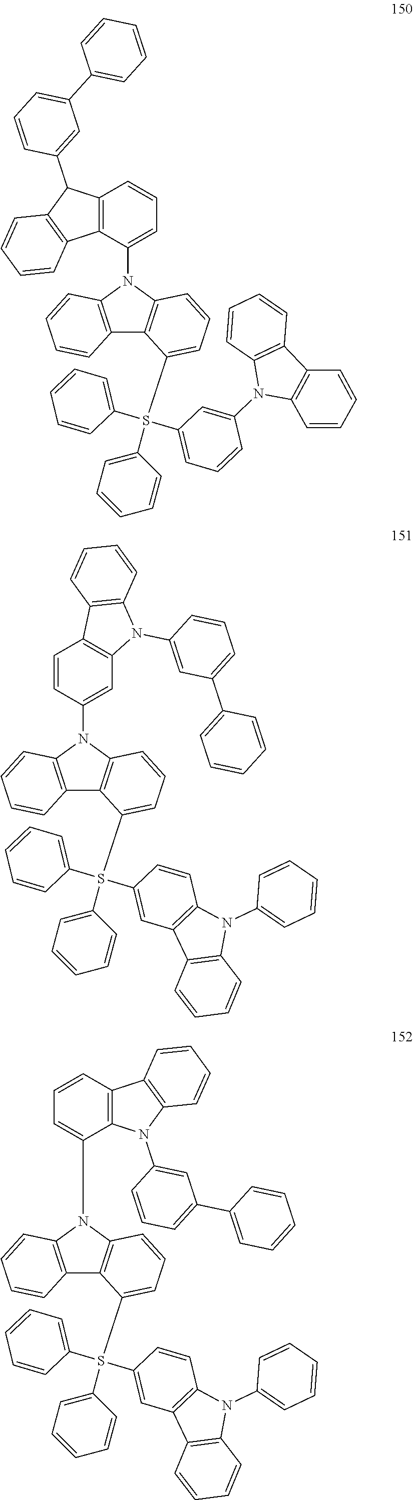 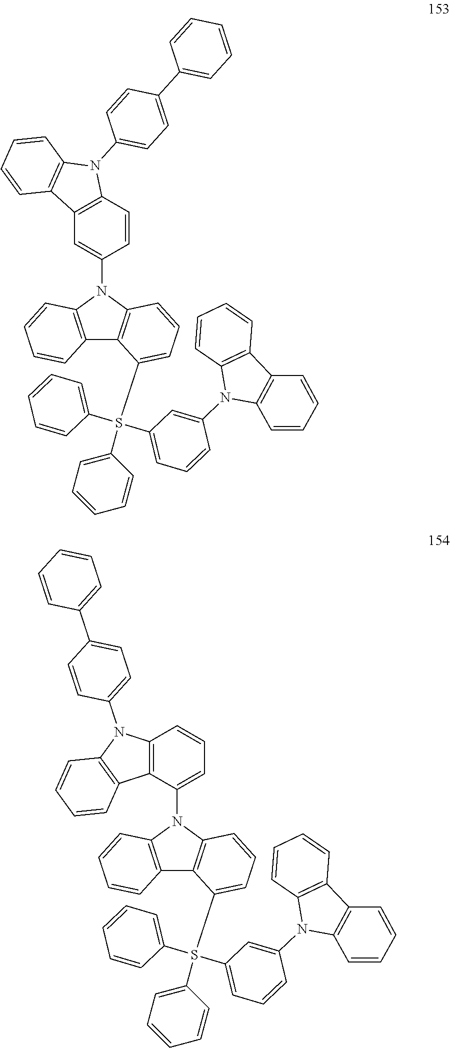 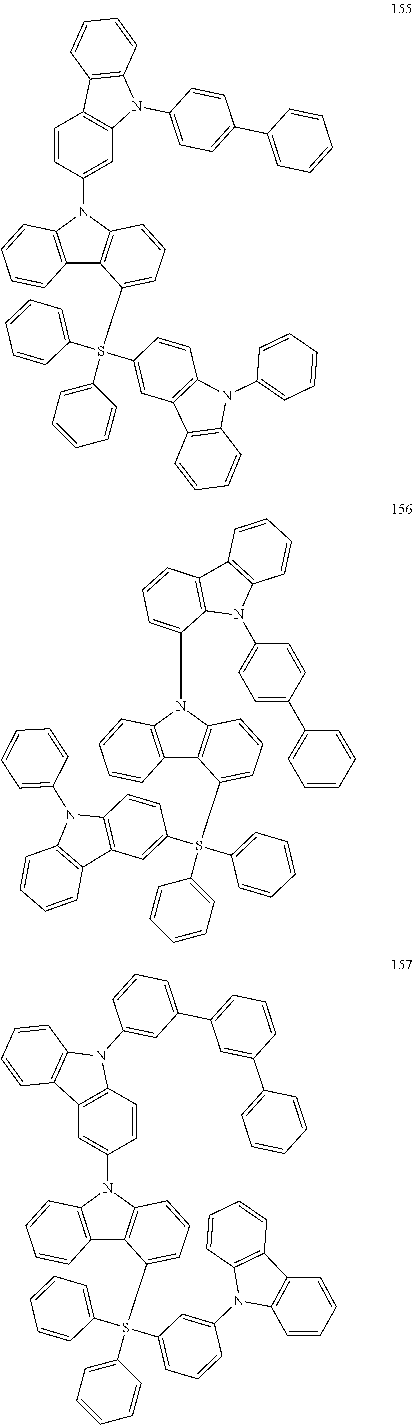 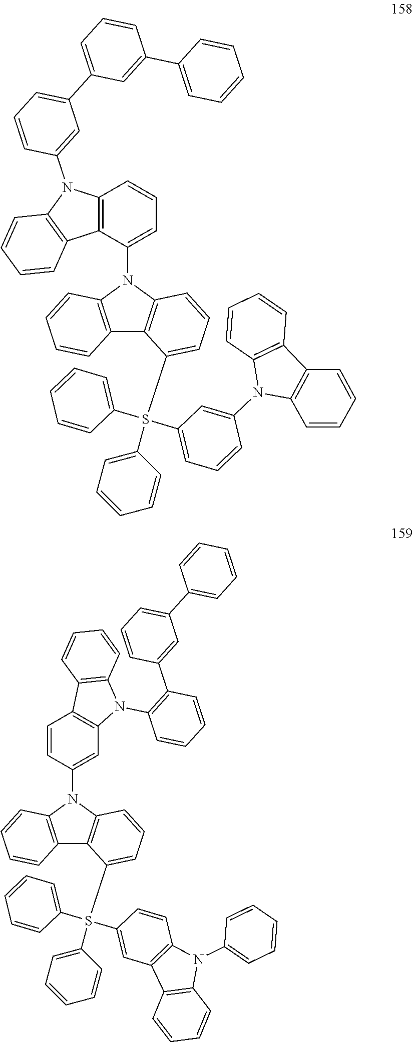 [SMILES: c1ccc(-c2cccc(-c3cccc(-n4c5ccccc5c5c(-n6c7ccccc7c7c(S(c8ccccc8)(c8ccccc8)c8cccc(-n9c%10ccccc%10c%10ccccc%109)c8)cccc76)cccc54)c3)c2)cc1 ; c1ccc(-c2cccc(-c3ccccc3-n3c4ccccc4c4ccc(-n5c6ccccc6c6c(S(c7ccccc7)(c7ccccc7)c7ccc8c(c7)c7ccccc7n8-c7ccccc7)cccc65)cc43)c2)cc1] 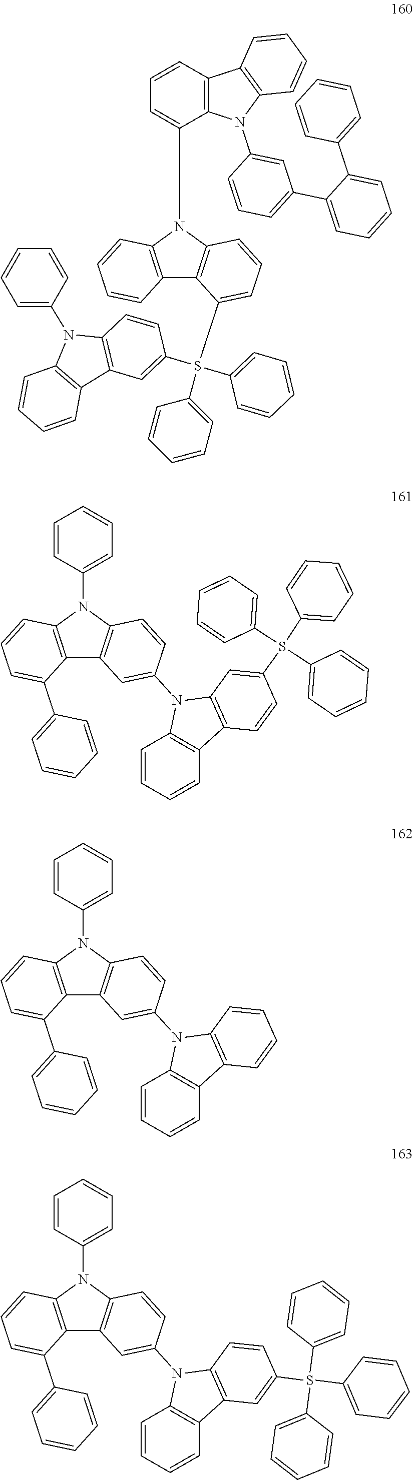 [SMILES: c1ccc(-c2ccccc2-c2cccc(-n3c4ccccc4c4cccc(-n5c6ccccc6c6c(S(c7ccccc7)(c7ccccc7)c7ccc8c(c7)c7ccccc7n8-c7ccccc7)cccc65)c43)c2)cc1 ; c1ccc(-c2cccc3c2c2cc(-n4c5ccccc5c5ccc(S(c6ccccc6)(c6ccccc6)c6ccccc6)cc54)ccc2n3-c2ccccc2)cc1 ; c1ccc(-c2cccc3c2c2cc(-n4c5ccccc5c5ccccc54)ccc2n3-c2ccccc2)cc1 ; c1ccc(-c2cccc3c2c2cc(-n4c5ccccc5c5cc(S(c6ccccc6)(c6ccccc6)c6ccccc6)ccc54)ccc2n3-c2ccccc2)cc1] 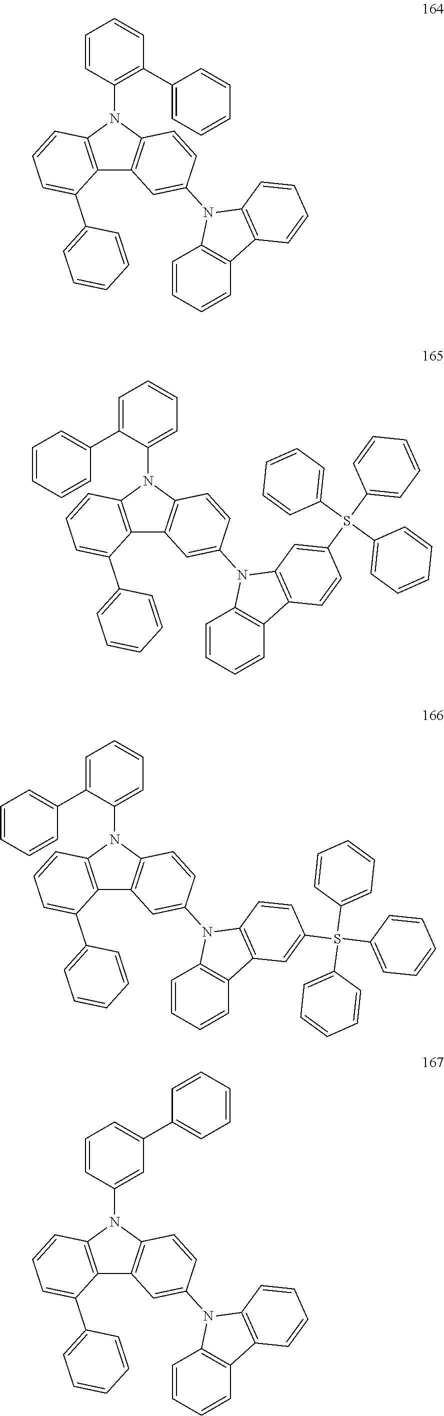 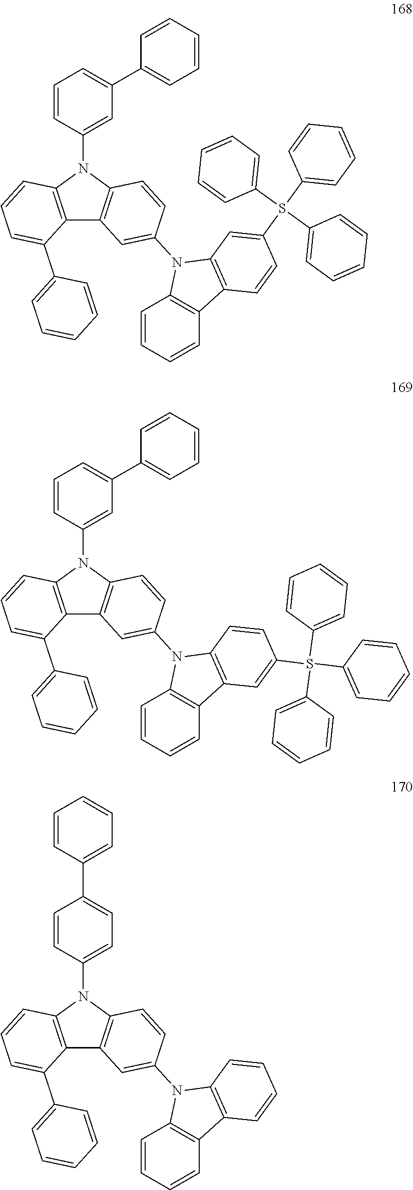 [SMILES: c1ccc(-c2cccc(-n3c4ccc(-n5c6ccccc6c6ccc(S(c7ccccc7)(c7ccccc7)c7ccccc7)cc65)cc4c4c(-c5ccccc5)cccc43)c2)cc1 ; c1ccc(-c2cccc(-n3c4ccc(-n5c6ccccc6c6cc(S(c7ccccc7)(c7ccccc7)c7ccccc7)ccc65)cc4c4c(-c5ccccc5)cccc43)c2)cc1 ; c1ccc(-c2ccc(-n3c4ccc(-n5c6ccccc6c6ccccc65)cc4c4c(-c5ccccc5)cccc43)cc2)cc1] 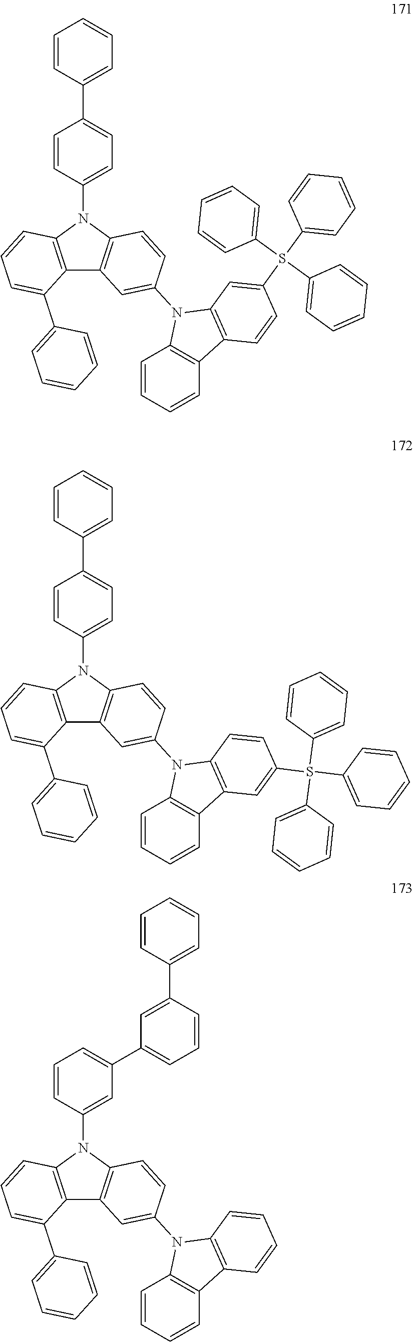 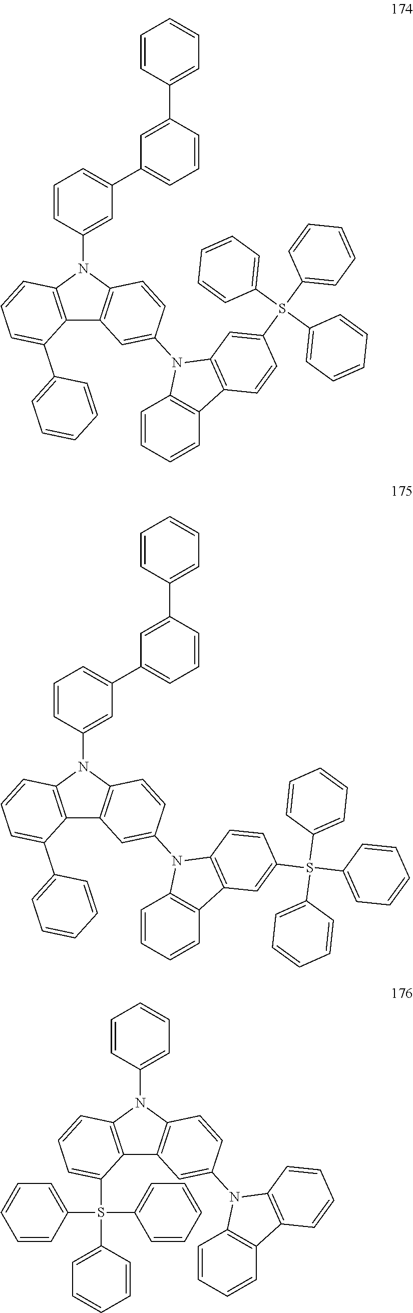 [SMILES: c1ccc(-c2cccc(-c3cccc(-n4c5ccc(-n6c7ccccc7c7ccc(S(c8ccccc8)(c8ccccc8)c8ccccc8)cc76)cc5c5c(-c6ccccc6)cccc54)c3)c2)cc1 ; c1ccc(-c2cccc(-c3cccc(-n4c5ccc(-n6c7ccccc7c7cc(S(c8ccccc8)(c8ccccc8)c8ccccc8)ccc76)cc5c5c(-c6ccccc6)cccc54)c3)c2)cc1 ; c1ccc(-n2c3ccc(-n4c5ccccc5c5ccccc54)cc3c3c(S(c4ccccc4)(c4ccccc4)c4ccccc4)cccc32)cc1] 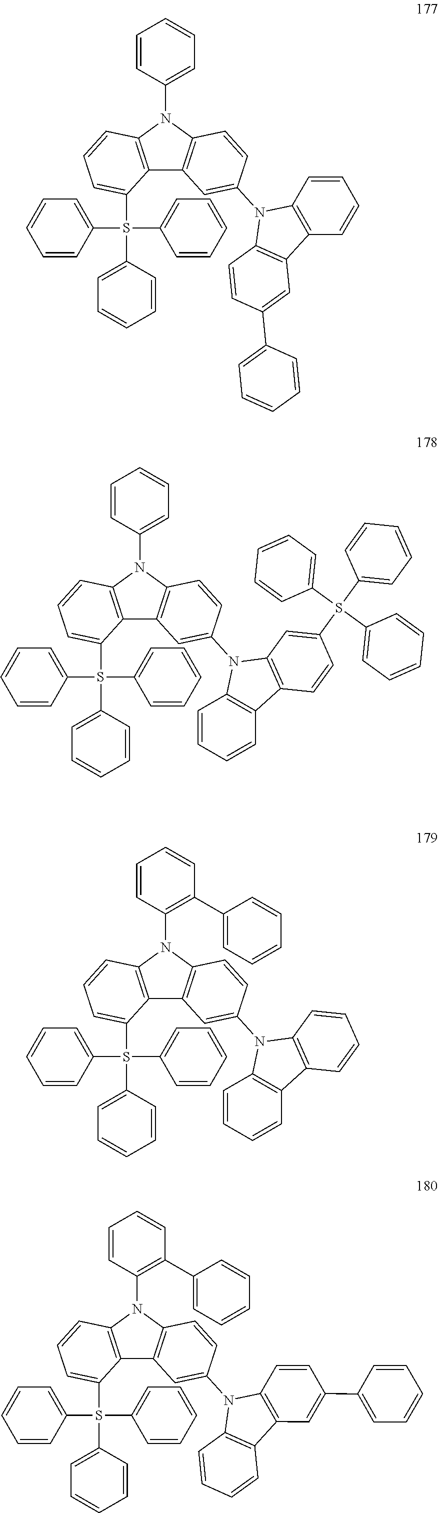 [SMILES: c1ccc(-c2ccc3c(c2)c2ccccc2n3-c2ccc3c(c2)c2c(S(c4ccccc4)(c4ccccc4)c4ccccc4)cccc2n3-c2ccccc2)cc1 ; c1ccc(-n2c3ccc(-n4c5ccccc5c5ccc(S(c6ccccc6)(c6ccccc6)c6ccccc6)cc54)cc3c3c(S(c4ccccc4)(c4ccccc4)c4ccccc4)cccc32)cc1 ; c1ccc(-c2ccccc2-n2c3ccc(-n4c5ccccc5c5ccccc54)cc3c3c(S(c4ccccc4)(c4ccccc4)c4ccccc4)cccc32)cc1 ; c1ccc(-c2ccc3c(c2)c2ccccc2n3-c2ccc3c(c2)c2c(S(c4ccccc4)(c4ccccc4)c4ccccc4)cccc2n3-c2ccccc2-c2ccccc2)cc1] 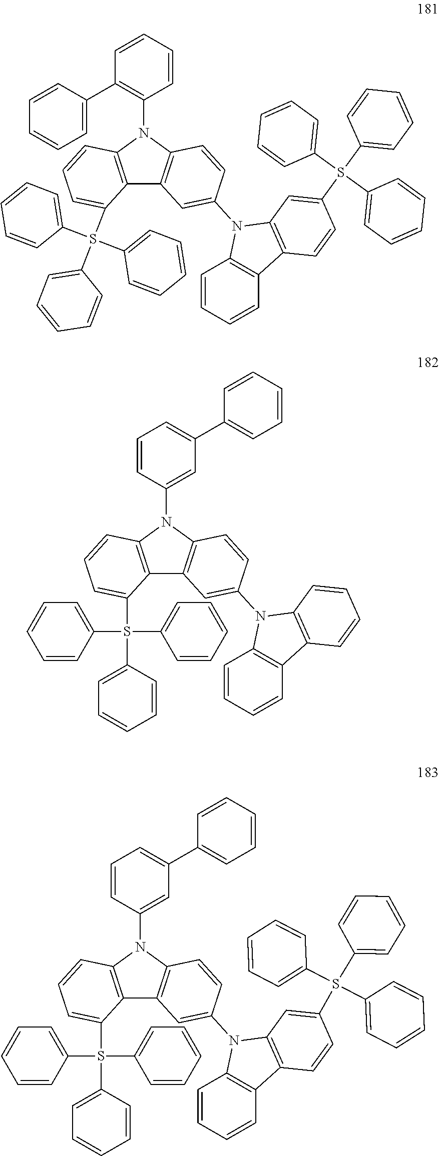 [SMILES: c1ccc(-c2ccccc2-n2c3ccc(-n4c5ccccc5c5ccc(S(c6ccccc6)(c6ccccc6)c6ccccc6)cc54)cc3c3c(S(c4ccccc4)(c4ccccc4)c4ccccc4)cccc32)cc1 ; c1ccc(-c2cccc(-n3c4ccc(-n5c6ccccc6c6ccccc65)cc4c4c(S(c5ccccc5)(c5ccccc5)c5ccccc5)cccc43)c2)cc1 ; c1ccc(-c2cccc(-n3c4ccc(-n5c6ccccc6c6ccc(S(c7ccccc7)(c7ccccc7)c7ccccc7)cc65)cc4c4c(S(c5ccccc5)(c5ccccc5)c5ccccc5)cccc43)c2)cc1] 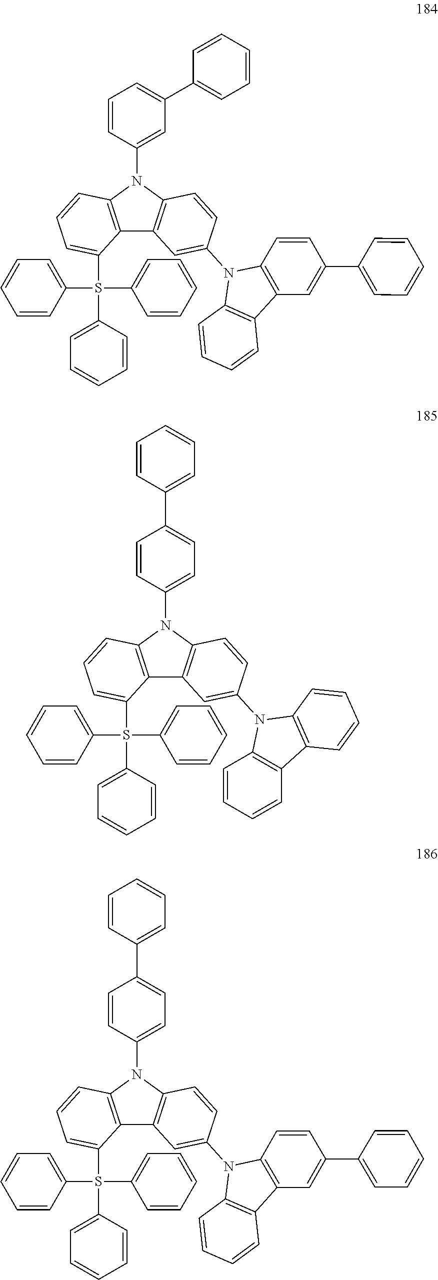 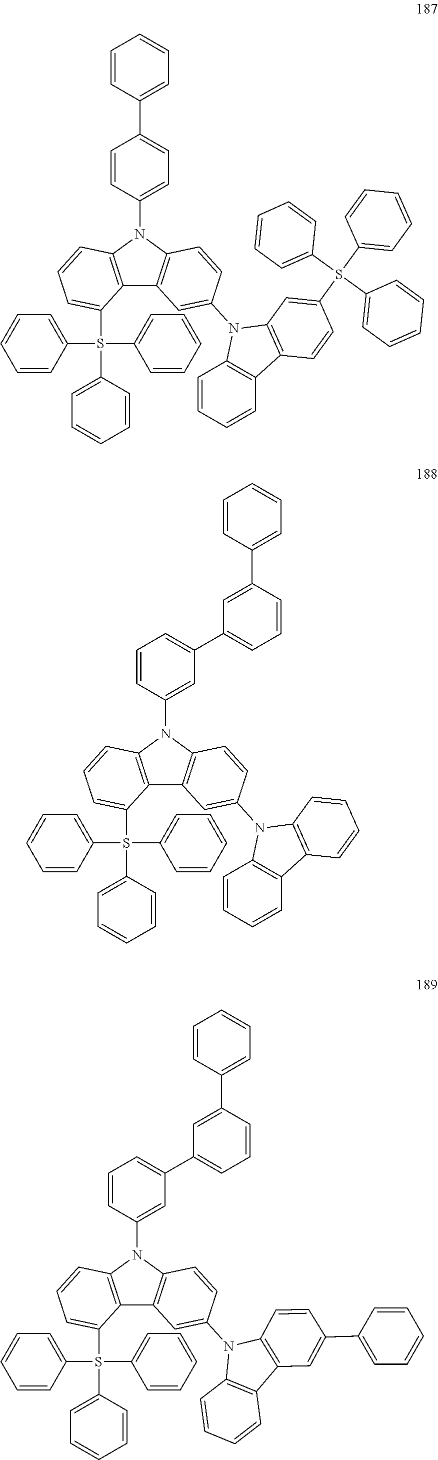 [SMILES: c1ccc(-c2ccc(-n3c4ccc(-n5c6ccccc6c6ccc(S(c7ccccc7)(c7ccccc7)c7ccccc7)cc65)cc4c4c(S(c5ccccc5)(c5ccccc5)c5ccccc5)cccc43)cc2)cc1 ; c1ccc(-c2cccc(-c3cccc(-n4c5ccc(-n6c7ccccc7c7ccccc76)cc5c5c(S(c6ccccc6)(c6ccccc6)c6ccccc6)cccc54)c3)c2)cc1 ; c1ccc(-c2cccc(-c3cccc(-n4c5ccc(-n6c7ccccc7c7cc(-c8ccccc8)ccc76)cc5c5c(S(c6ccccc6)(c6ccccc6)c6ccccc6)cccc54)c3)c2)cc1] 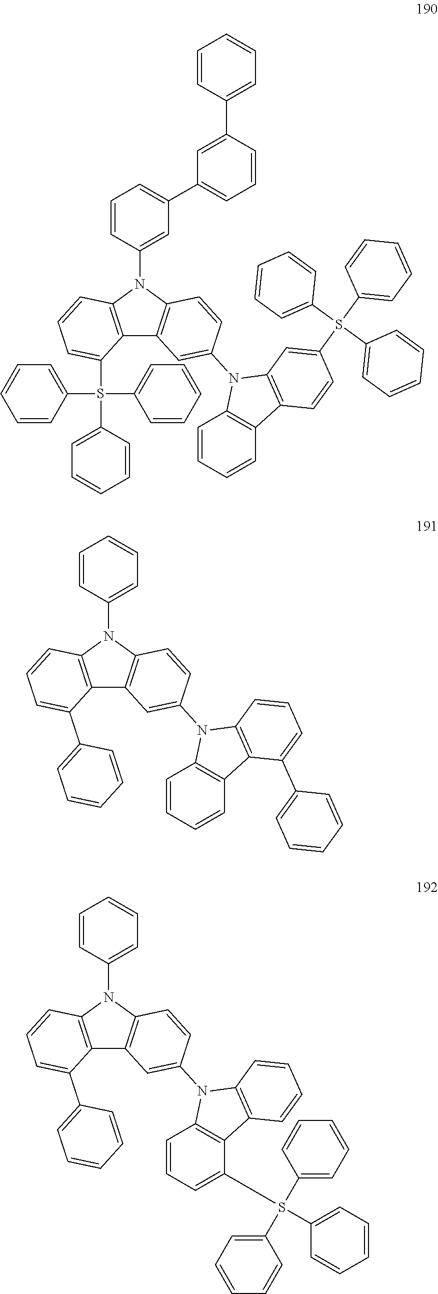 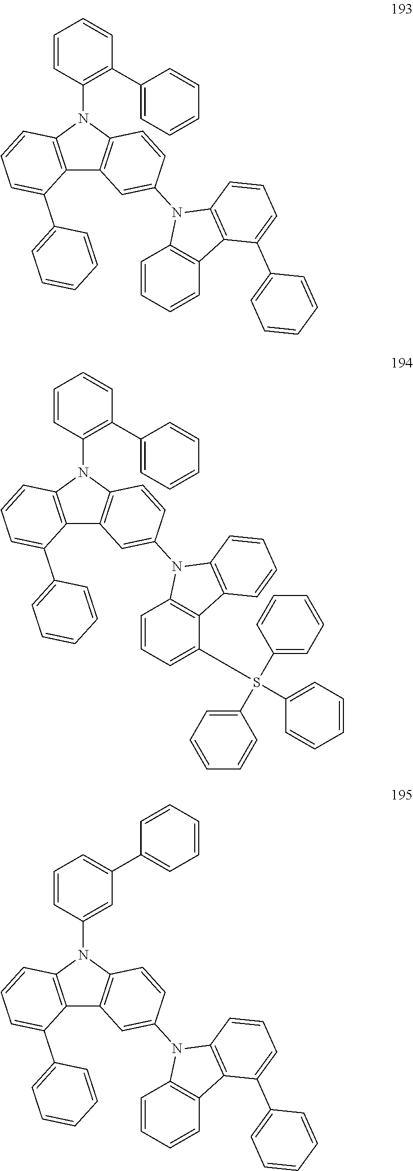 [SMILES: c1ccc(-c2ccccc2-n2c3ccc(-n4c5ccccc5c5c(-c6ccccc6)cccc54)cc3c3c(-c4ccccc4)cccc32)cc1 ; c1ccc(-c2ccccc2-n2c3ccc(-n4c5ccccc5c5c(S(c6ccccc6)(c6ccccc6)c6ccccc6)cccc54)cc3c3c(-c4ccccc4)cccc32)cc1 ; c1ccc(-c2cccc(-n3c4ccc(-n5c6ccccc6c6c(-c7ccccc7)cccc65)cc4c4c(-c5ccccc5)cccc43)c2)cc1] 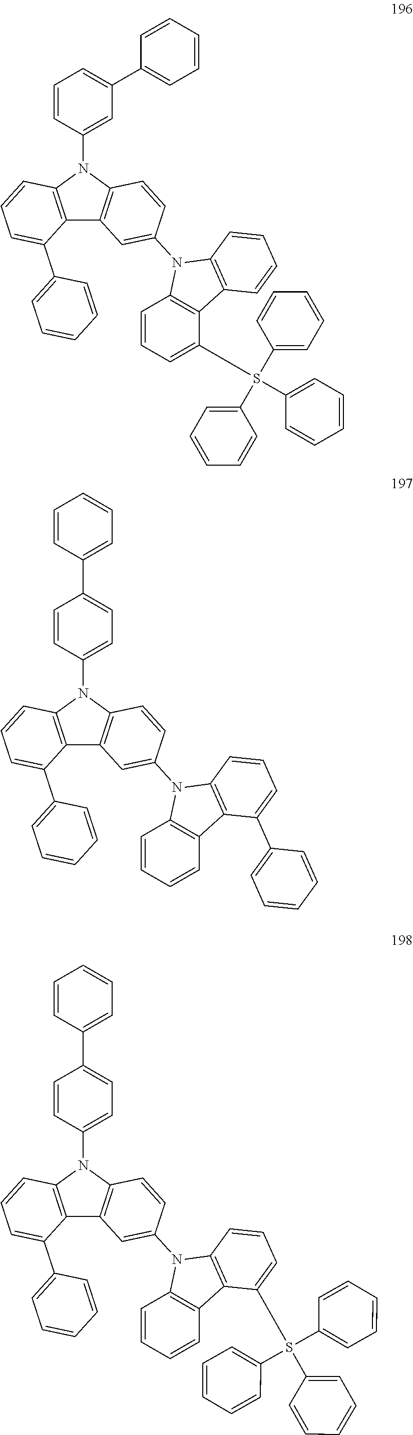 [SMILES: c1ccc(-c2cccc(-n3c4ccc(-n5c6ccccc6c6c(S(c7ccccc7)(c7ccccc7)c7ccccc7)cccc65)cc4c4c(-c5ccccc5)cccc43)c2)cc1 ; c1ccc(-c2ccc(-n3c4ccc(-n5c6ccccc6c6c(-c7ccccc7)cccc65)cc4c4c(-c5ccccc5)cccc43)cc2)cc1 ; c1ccc(-c2ccc(-n3c4ccc(-n5c6ccccc6c6c(S(c7ccccc7)(c7ccccc7)c7ccccc7)cccc65)cc4c4c(-c5ccccc5)cccc43)cc2)cc1] 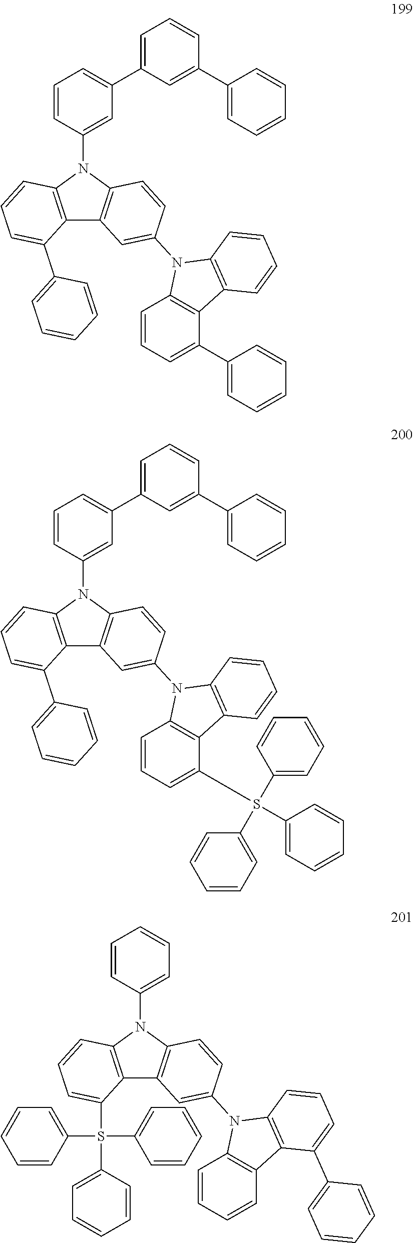 [SMILES: c1ccc(-c2cccc(-c3cccc(-n4c5ccc(-n6c7ccccc7c7c(-c8ccccc8)cccc76)cc5c5c(-c6ccccc6)cccc54)c3)c2)cc1 ; c1ccc(-c2cccc(-c3cccc(-n4c5ccc(-n6c7ccccc7c7c(S(c8ccccc8)(c8ccccc8)c8ccccc8)cccc76)cc5c5c(-c6ccccc6)cccc54)c3)c2)cc1 ; c1ccc(-c2cccc3c2c2ccccc2n3-c2ccc3c(c2)c2c(S(c4ccccc4)(c4ccccc4)c4ccccc4)cccc2n3-c2ccccc2)cc1] 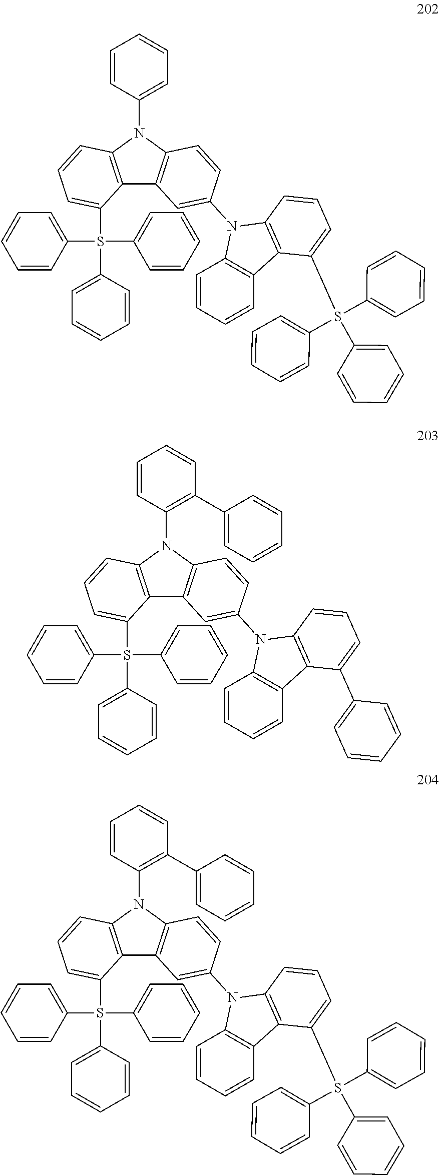 [SMILES: c1ccc(-n2c3ccc(-n4c5ccccc5c5c(CS(c6ccccc6)(c6ccccc6)c6ccccc6)cccc54)cc3c3c(S(c4ccccc4)(c4ccccc4)c4ccccc4)cccc32)cc1 ; c1ccc(-c2ccccc2-n2c3ccc(-n4c5ccccc5c5c(-c6ccccc6)cccc54)cc3c3c(S(c4ccccc4)(c4ccccc4)c4ccccc4)cccc32)cc1 ; c1ccc(-c2ccccc2-n2c3ccc(-n4c5ccccc5c5c(CS(c6ccccc6)(c6ccccc6)c6ccccc6)cccc54)cc3c3c(S(c4ccccc4)(c4ccccc4)c4ccccc4)cccc32)cc1] 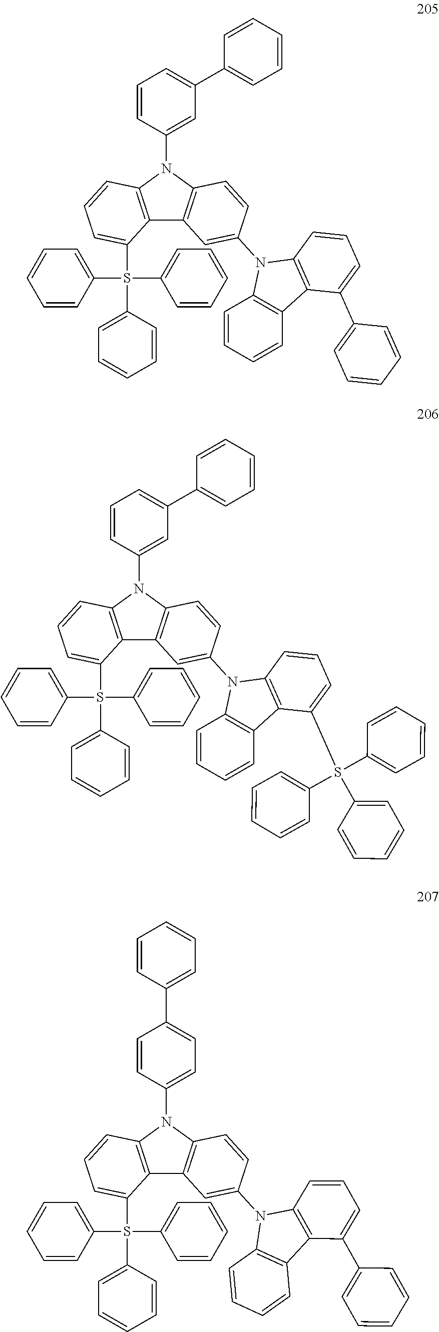 [SMILES: c1ccc(-c2cccc(-n3c4ccc(-n5c6ccccc6c6c(-c7ccccc7)cccc65)cc4c4c(S(c5ccccc5)(c5ccccc5)c5ccccc5)cccc43)c2)cc1 ; c1ccc(-c2cccc(-n3c4ccc(-n5c6ccccc6c6c(CS(c7ccccc7)(c7ccccc7)c7ccccc7)cccc65)cc4c4c(S(c5ccccc5)(c5ccccc5)c5ccccc5)cccc43)c2)cc1 ; c1ccc(-c2ccc(-n3c4ccc(-n5c6ccccc6c6c(-c7ccccc7)cccc65)cc4c4c(S(c5ccccc5)(c5ccccc5)c5ccccc5)cccc43)cc2)cc1] 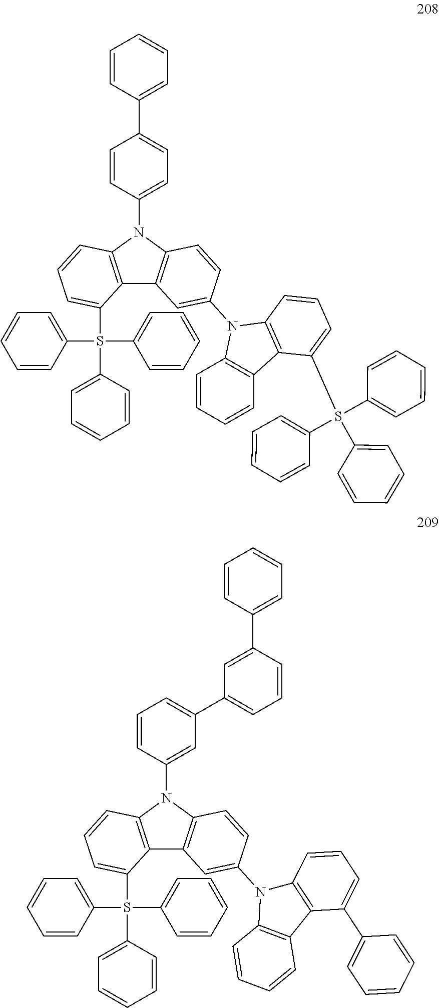 [SMILES: c1ccc(-c2ccc(-n3c4ccc(-n5c6ccccc6c6c(CS(c7ccccc7)(c7ccccc7)c7ccccc7)cccc65)cc4c4c(S(c5ccccc5)(c5ccccc5)c5ccccc5)cccc43)cc2)cc1 ; c1ccc(-c2cccc(-c3cccc(-n4c5ccc(-n6c7ccccc7c7c(-c8ccccc8)cccc76)cc5c5c(S(c6ccccc6)(c6ccccc6)c6ccccc6)cccc54)c3)c2)cc1] 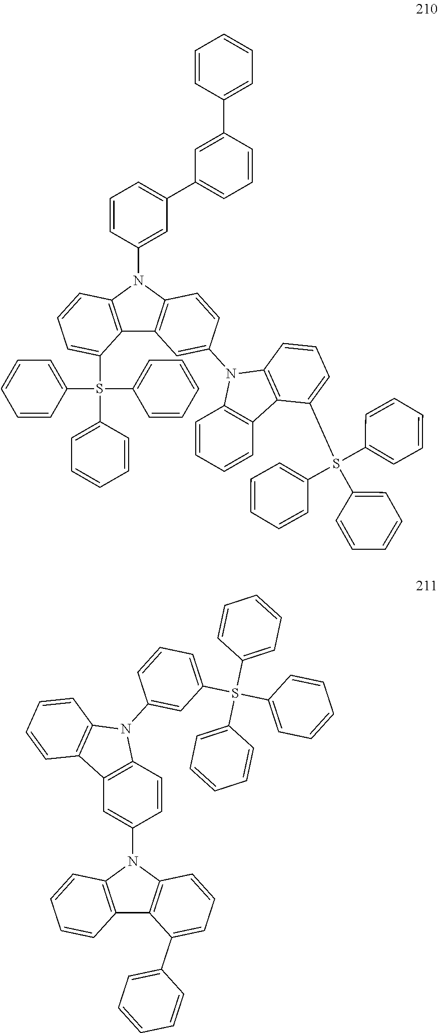 [SMILES: c1ccc(-c2cccc(-c3cccc(-n4c5ccc(-n6c7ccccc7c7c(CS(c8ccccc8)(c8ccccc8)c8ccccc8)cccc76)cc5c5c(S(c6ccccc6)(c6ccccc6)c6ccccc6)cccc54)c3)c2)cc1 ; c1ccc(-c2cccc3c2c2ccccc2n3-c2ccc3c(c2)c2ccccc2n3-c2cccc(S(c3ccccc3)(c3ccccc3)c3ccccc3)c2)cc1] 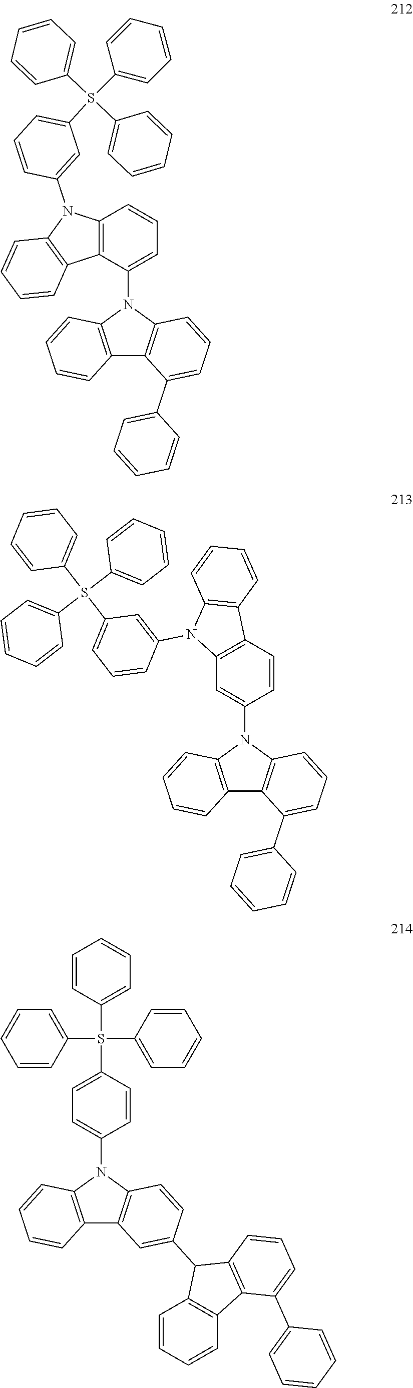 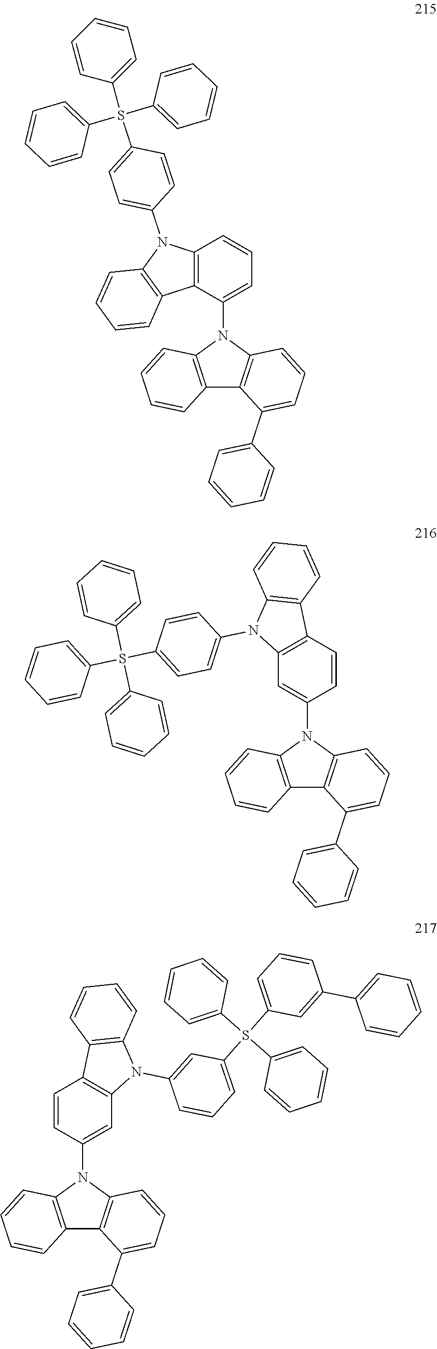 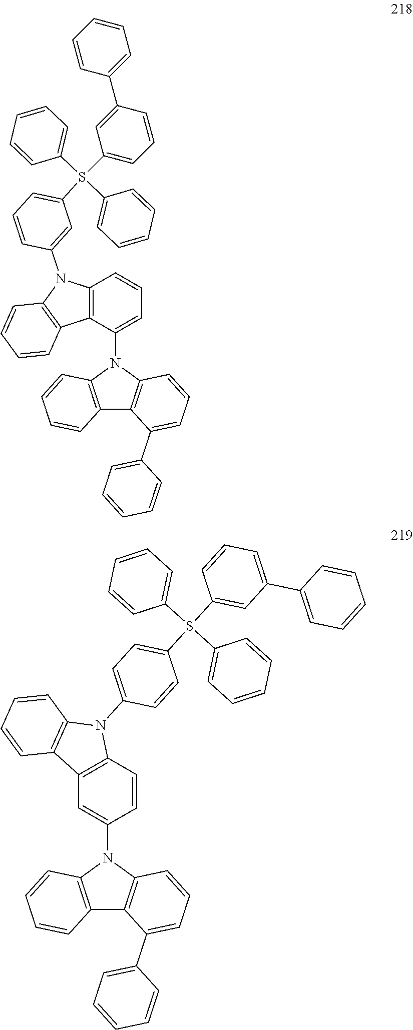 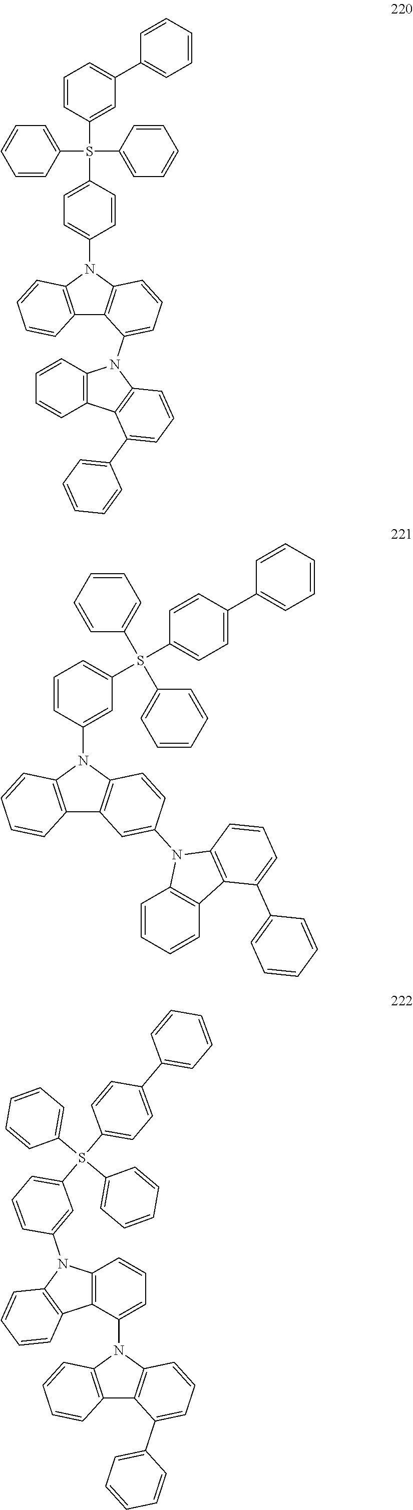 [SMILES: c1ccc(-c2cccc(S(c3ccccc3)(c3ccccc3)c3ccc(-n4c5ccccc5c5c(-n6c7ccccc7c7c(-c8ccccc8)cccc76)cccc54)cc3)c2)cc1 ; c1ccc(-c2ccc(S(c3ccccc3)(c3ccccc3)c3cccc(-n4c5ccccc5c5cc(-n6c7ccccc7c7c(-c8ccccc8)cccc76)ccc54)c3)cc2)cc1 ; c1ccc(-c2ccc(S(c3ccccc3)(c3ccccc3)c3cccc(-n4c5ccccc5c5c(-n6c7ccccc7c7c(-c8ccccc8)cccc76)cccc54)c3)cc2)cc1] 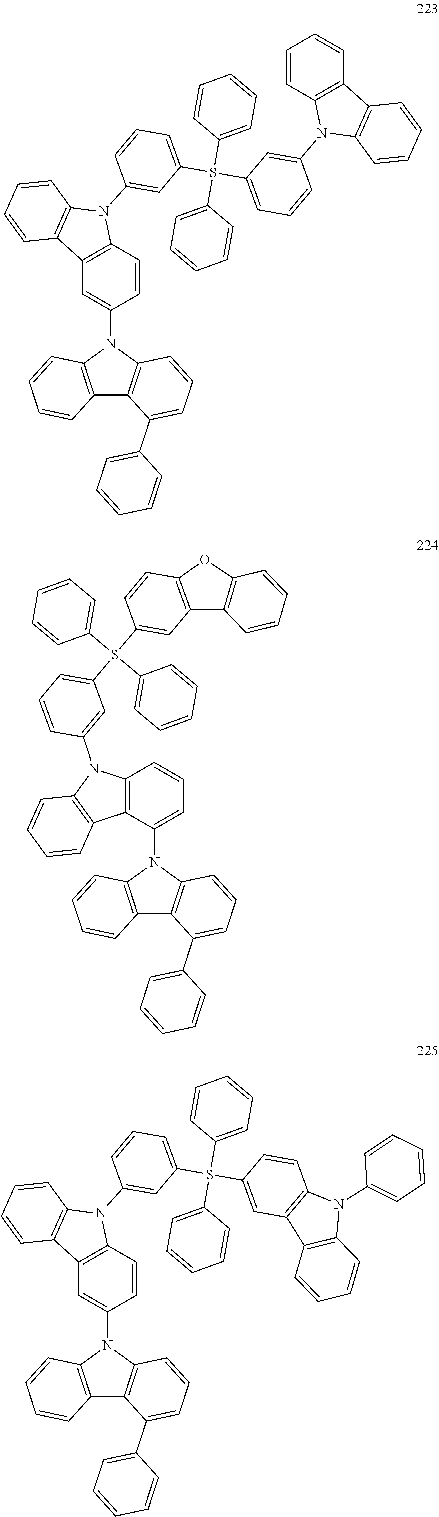 [SMILES: c1ccc(-c2cccc3c2c2ccccc2n3-c2ccc3c(c2)c2ccccc2n3-c2cccc(S(c3ccccc3)(c3ccccc3)c3cccc(-n4c5ccccc5c5ccccc54)c3)c2)cc1 ; c1ccc(-c2cccc3c2c2ccccc2n3-c2cccc3c2c2ccccc2n3-c2cccc(S(c3ccccc3)(c3ccccc3)c3ccc4oc5ccccc5c4c3)c2)cc1 ; c1ccc(-c2cccc3c2c2ccccc2n3-c2ccc3c(c2)c2ccccc2n3-c2cccc(S(c3ccccc3)(c3ccccc3)c3ccc4c(c3)c3ccccc3n4-c3ccccc3)c2)cc1] 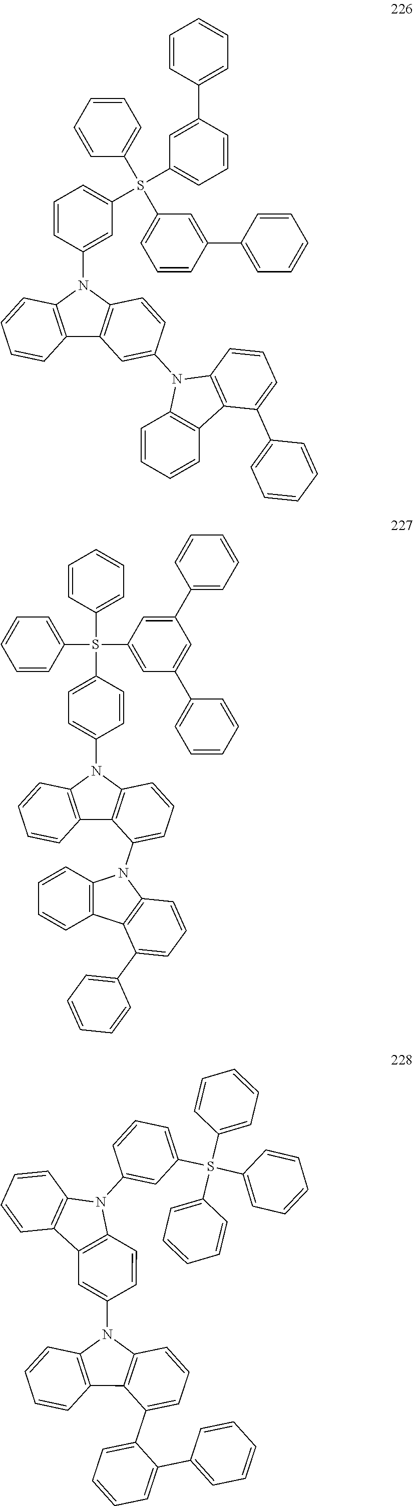 [SMILES: c1ccc(-c2cccc(S(c3ccccc3)(c3cccc(-c4ccccc4)c3)c3cccc(-n4c5ccccc5c5cc(-n6c7ccccc7c7c(-c8ccccc8)cccc76)ccc54)c3)c2)cc1 ; c1ccc(-c2cc(-c3ccccc3)cc(S(c3ccccc3)(c3ccccc3)c3ccc(-n4c5ccccc5c5c(-n6c7ccccc7c7c(-c8ccccc8)cccc76)cccc54)cc3)c2)cc1 ; c1ccc(-c2ccccc2-c2cccc3c2c2ccccc2n3-c2ccc3c(c2)c2ccccc2n3-c2cccc(S(c3ccccc3)(c3ccccc3)c3ccccc3)c2)cc1] 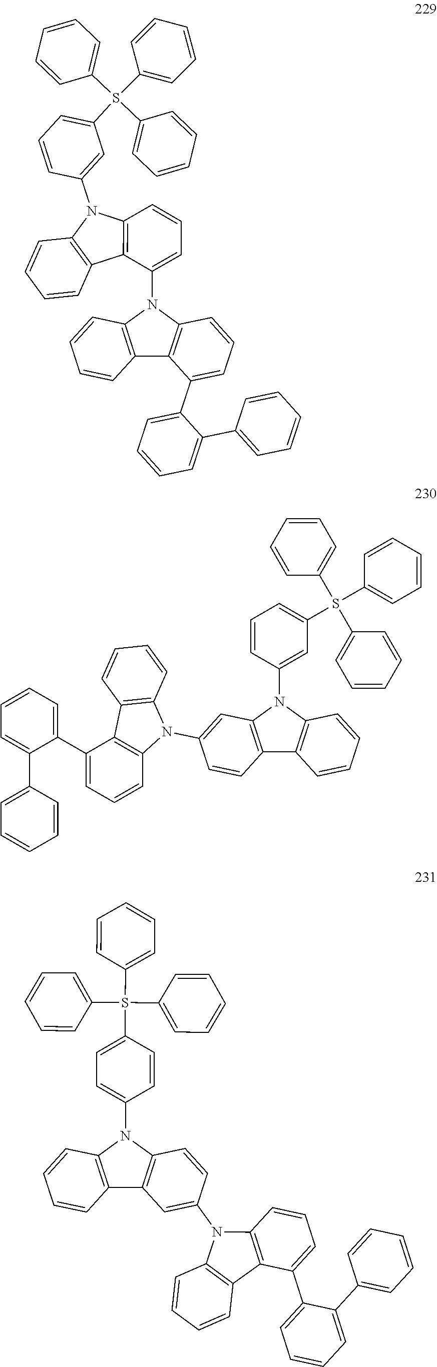 [SMILES: c1ccc(-c2ccccc2-c2cccc3c2c2ccccc2n3-c2cccc3c2c2ccccc2n3-c2cccc(S(c3ccccc3)(c3ccccc3)c3ccccc3)c2)cc1 ; c1ccc(-c2ccccc2-c2cccc3c2c2ccccc2n3-c2ccc3c4ccccc4n(-c4cccc(S(c5ccccc5)(c5ccccc5)c5ccccc5)c4)c3c2)cc1 ; c1ccc(-c2ccccc2-c2cccc3c2c2ccccc2n3-c2ccc3c(c2)c2ccccc2n3-c2ccc(S(c3ccccc3)(c3ccccc3)c3ccccc3)cc2)cc1] 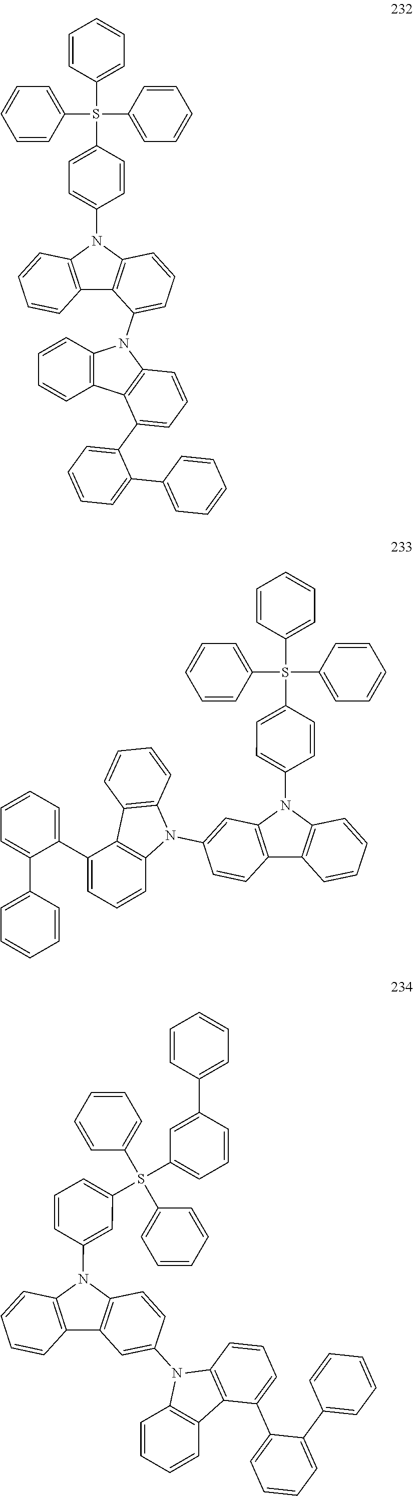 [SMILES: c1ccc(-c2ccccc2-c2cccc3c2c2ccccc2n3-c2cccc3c2c2ccccc2n3-c2ccc(S(c3ccccc3)(c3ccccc3)c3ccccc3)cc2)cc1 ; c1ccc(-c2ccccc2-c2cccc3c2c2ccccc2n3-c2ccc3c4ccccc4n(-c4ccc(S(c5ccccc5)(c5ccccc5)c5ccccc5)cc4)c3c2)cc1 ; c1ccc(-c2cccc(S(c3ccccc3)(c3ccccc3)c3cccc(-n4c5ccccc5c5cc(-n6c7ccccc7c7c(-c8ccccc8-c8ccccc8)cccc76)ccc54)c3)c2)cc1] 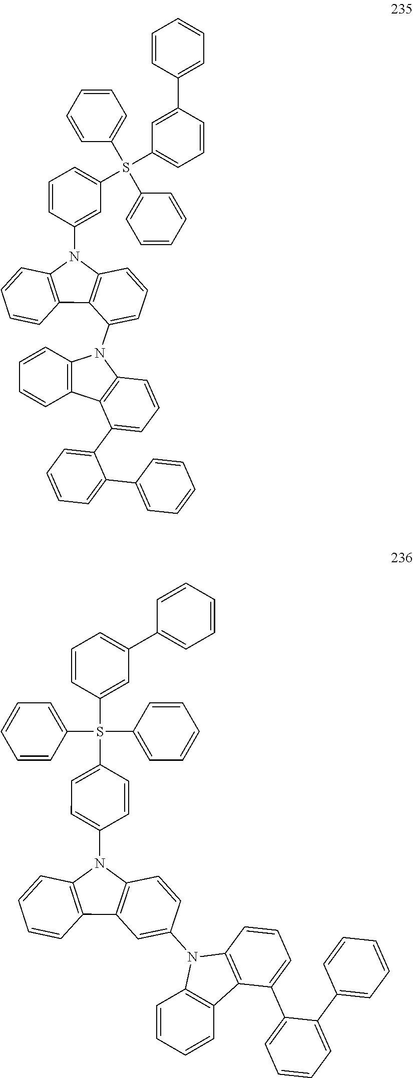 [SMILES: c1ccc(-c2cccc(S(c3ccccc3)(c3ccccc3)c3cccc(-n4c5ccccc5c5c(-n6c7ccccc7c7c(-c8ccccc8-c8ccccc8)cccc76)cccc54)c3)c2)cc1 ; c1ccc(-c2cccc(S(c3ccccc3)(c3ccccc3)c3ccc(-n4c5ccccc5c5cc(-n6c7ccccc7c7c(-c8ccccc8-c8ccccc8)cccc76)ccc54)cc3)c2)cc1] 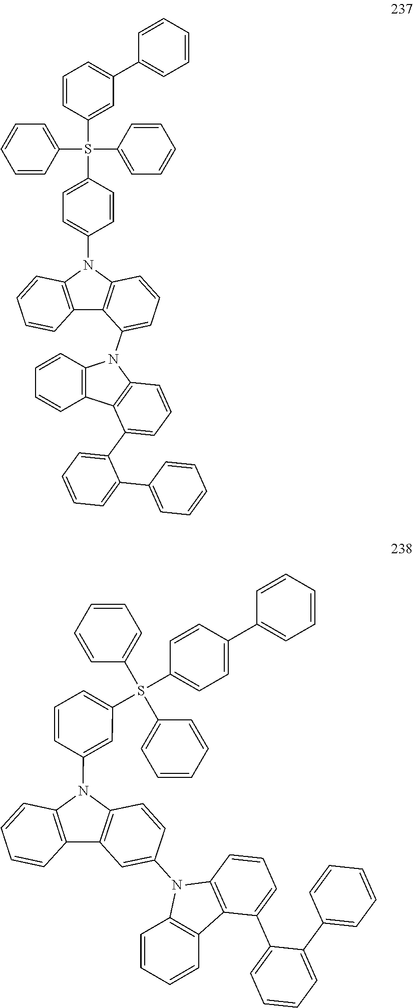 [SMILES: c1ccc(-c2cccc(S(c3ccccc3)(c3ccccc3)c3ccc(-n4c5ccccc5c5c(-n6c7ccccc7c7c(-c8ccccc8-c8ccccc8)cccc76)cccc54)cc3)c2)cc1 ; c1ccc(-c2ccc(S(c3ccccc3)(c3ccccc3)c3cccc(-n4c5ccccc5c5cc(-n6c7ccccc7c7c(-c8ccccc8-c8ccccc8)cccc76)ccc54)c3)cc2)cc1] 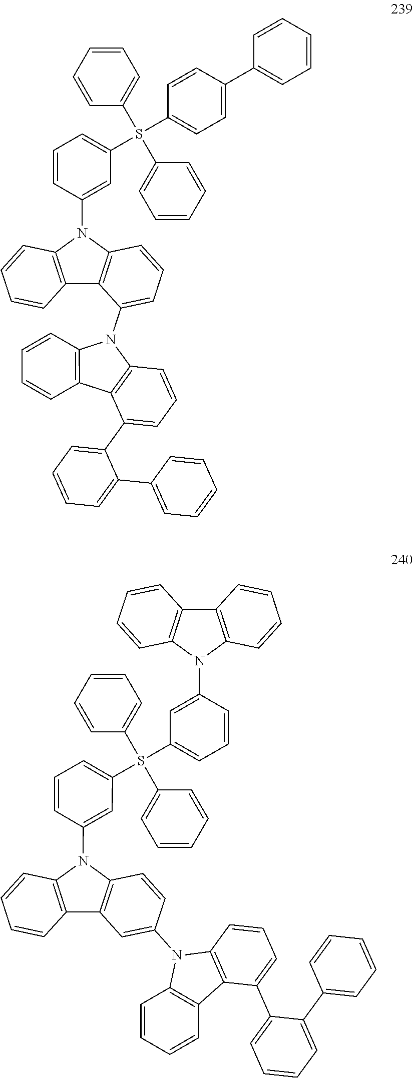 [SMILES: c1ccc(-c2ccc(S(c3ccccc3)(c3ccccc3)c3cccc(-n4c5ccccc5c5c(-n6c7ccccc7c7c(-c8ccccc8-c8ccccc8)cccc76)cccc54)c3)cc2)cc1 ; c1ccc(-c2ccccc2-c2cccc3c2c2ccccc2n3-c2ccc3c(c2)c2ccccc2n3-c2cccc(S(c3ccccc3)(c3ccccc3)c3cccc(-n4c5ccccc5c5ccccc54)c3)c2)cc1] 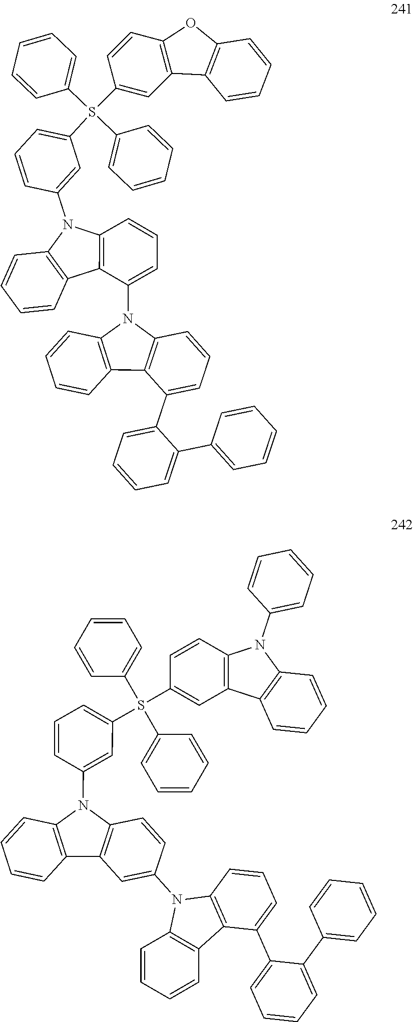 [SMILES: c1ccc(-c2ccccc2-c2cccc3c2c2ccccc2n3-c2cccc3c2c2ccccc2n3-c2cccc(S(c3ccccc3)(c3ccccc3)c3ccc4oc5ccccc5c4c3)c2)cc1 ; c1ccc(-c2ccccc2-c2cccc3c2c2ccccc2n3-c2ccc3c(c2)c2ccccc2n3-c2cccc(S(c3ccccc3)(c3ccccc3)c3ccc4c(c3)c3ccccc3n4-c3ccccc3)c2)cc1] 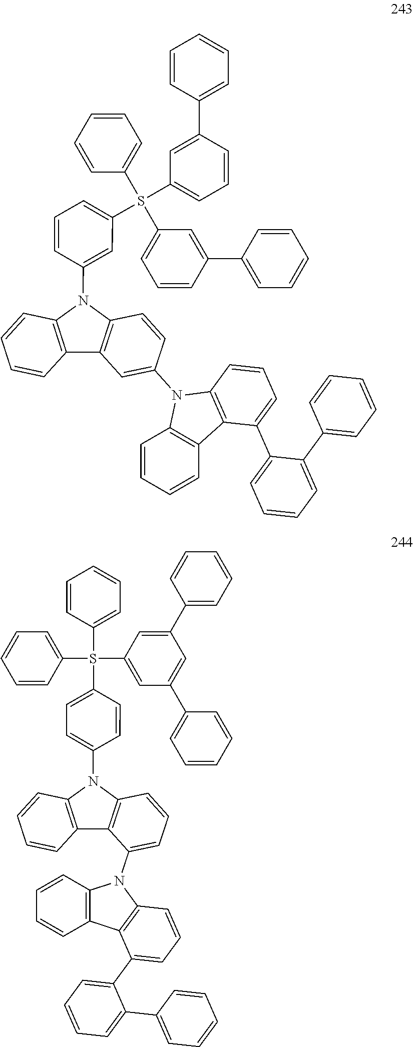 [SMILES: c1ccc(-c2cccc(S(c3ccccc3)(c3cccc(-c4ccccc4)c3)c3cccc(-n4c5ccccc5c5cc(-n6c7ccccc7c7c(-c8ccccc8-c8ccccc8)cccc76)ccc54)c3)c2)cc1 ; c1ccc(-c2cc(-c3ccccc3)cc(S(c3ccccc3)(c3ccccc3)c3ccc(-n4c5ccccc5c5c(-n6c7ccccc7c7c(-c8ccccc8-c8ccccc8)cccc76)cccc54)cc3)c2)cc1] 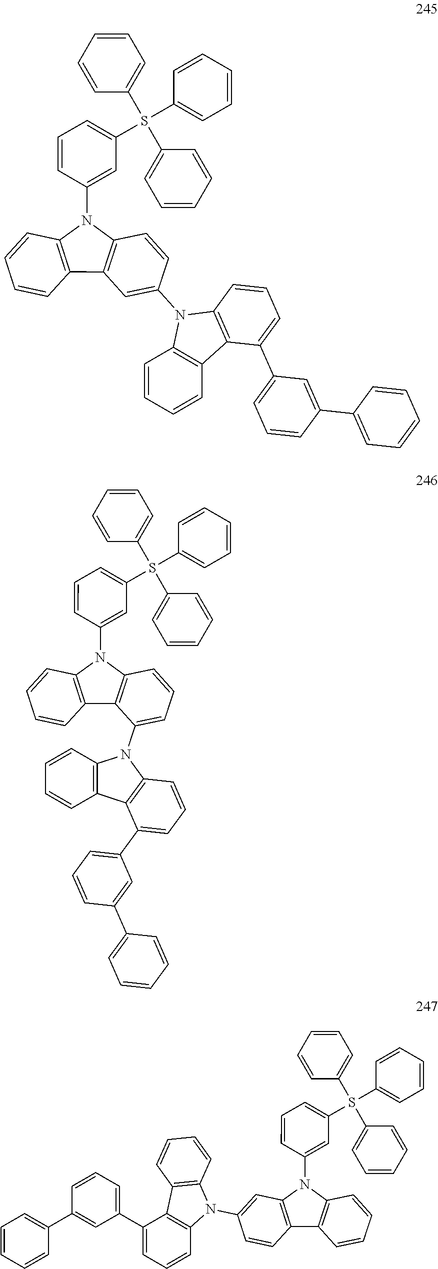 [SMILES: c1ccc(-c2cccc(-c3cccc4c3c3ccccc3n4-c3ccc4c(c3)c3ccccc3n4-c3cccc(S(c4ccccc4)(c4ccccc4)c4ccccc4)c3)c2)cc1 ; c1ccc(-c2cccc(-c3cccc4c3c3ccccc3n4-c3cccc4c3c3ccccc3n4-c3cccc(S(c4ccccc4)(c4ccccc4)c4ccccc4)c3)c2)cc1 ; c1ccc(-c2cccc(-c3cccc4c3c3ccccc3n4-c3ccc4c5ccccc5n(-c5cccc(S(c6ccccc6)(c6ccccc6)c6ccccc6)c5)c4c3)c2)cc1] 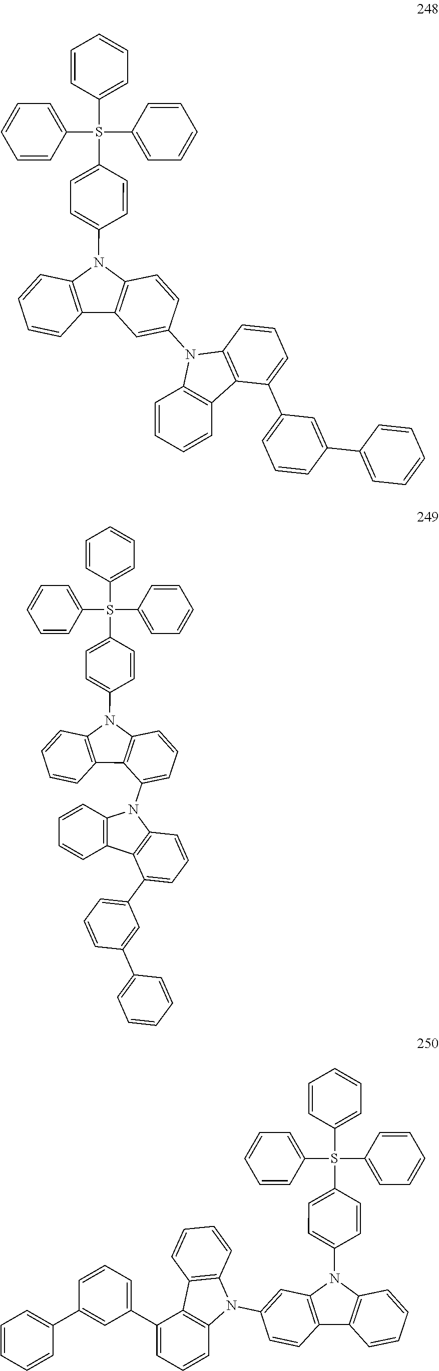 [SMILES: c1ccc(-c2cccc(-c3cccc4c3c3ccccc3n4-c3ccc4c(c3)c3ccccc3n4-c3ccc(S(c4ccccc4)(c4ccccc4)c4ccccc4)cc3)c2)cc1 ; c1ccc(-c2cccc(-c3cccc4c3c3ccccc3n4-c3cccc4c3c3ccccc3n4-c3ccc(S(c4ccccc4)(c4ccccc4)c4ccccc4)cc3)c2)cc1 ; c1ccc(-c2cccc(-c3cccc4c3c3ccccc3n4-c3ccc4c5ccccc5n(-c5ccc(S(c6ccccc6)(c6ccccc6)c6ccccc6)cc5)c4c3)c2)cc1] 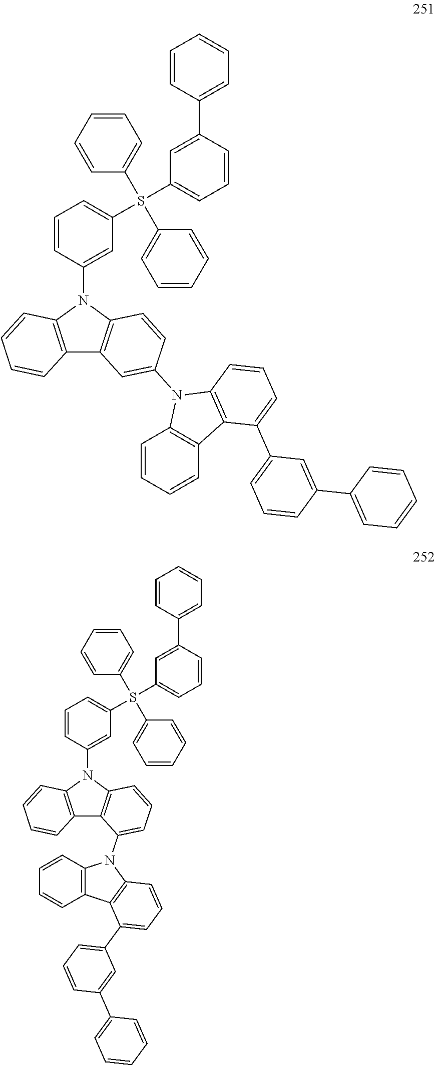 [SMILES: c1ccc(-c2cccc(-c3cccc4c3c3ccccc3n4-c3ccc4c(c3)c3ccccc3n4-c3cccc(S(c4ccccc4)(c4ccccc4)c4cccc(-c5ccccc5)c4)c3)c2)cc1 ; c1ccc(-c2cccc(-c3cccc4c3c3ccccc3n4-c3cccc4c3c3ccccc3n4-c3cccc(S(c4ccccc4)(c4ccccc4)c4cccc(-c5ccccc5)c4)c3)c2)cc1] 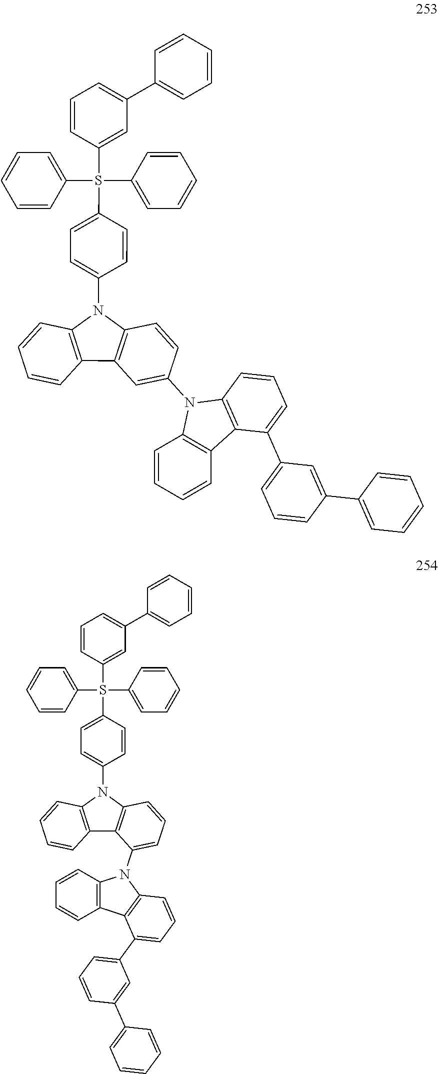 [SMILES: c1ccc(-c2cccc(-c3cccc4c3c3ccccc3n4-c3ccc4c(c3)c3ccccc3n4-c3ccc(S(c4ccccc4)(c4ccccc4)c4cccc(-c5ccccc5)c4)cc3)c2)cc1 ; c1ccc(-c2cccc(-c3cccc4c3c3ccccc3n4-c3cccc4c3c3ccccc3n4-c3ccc(S(c4ccccc4)(c4ccccc4)c4cccc(-c5ccccc5)c4)cc3)c2)cc1] 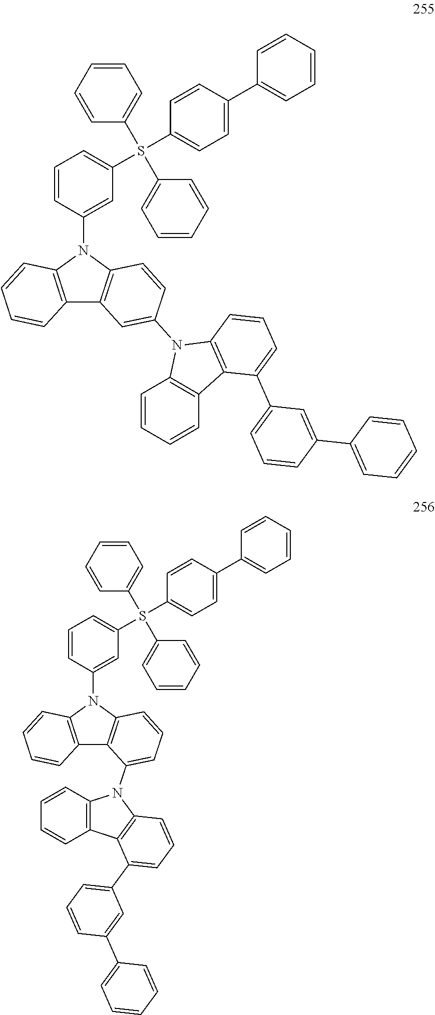 [SMILES: c1ccc(-c2ccc(S(c3ccccc3)(c3ccccc3)c3cccc(-n4c5ccccc5c5cc(-n6c7ccccc7c7c(-c8cccc(-c9ccccc9)c8)cccc76)ccc54)c3)cc2)cc1 ; c1ccc(-c2ccc(S(c3ccccc3)(c3ccccc3)c3cccc(-n4c5ccccc5c5c(-n6c7ccccc7c7c(-c8cccc(-c9ccccc9)c8)cccc76)cccc54)c3)cc2)cc1] 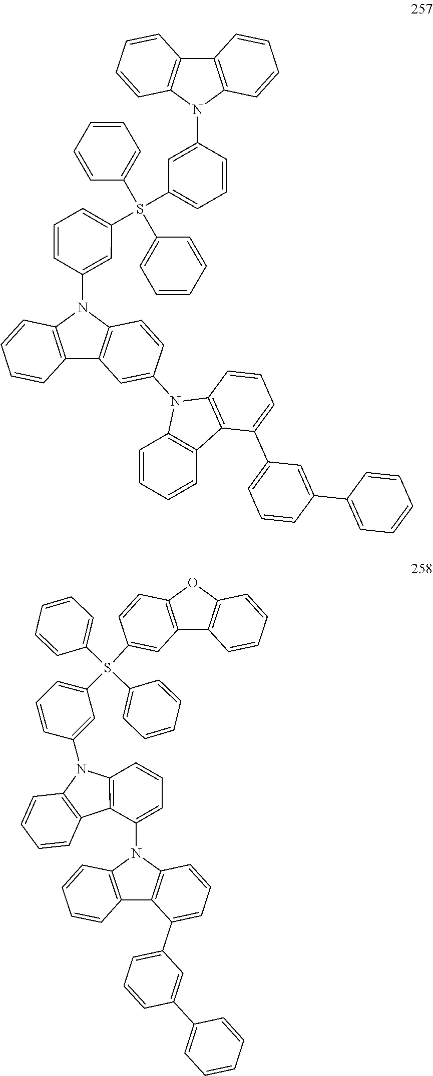 [SMILES: c1ccc(-c2cccc(-c3cccc4c3c3ccccc3n4-c3ccc4c(c3)c3ccccc3n4-c3cccc(S(c4ccccc4)(c4ccccc4)c4cccc(-n5c6ccccc6c6ccccc65)c4)c3)c2)cc1 ; c1ccc(-c2cccc(-c3cccc4c3c3ccccc3n4-c3cccc4c3c3ccccc3n4-c3cccc(S(c4ccccc4)(c4ccccc4)c4ccc5oc6ccccc6c5c4)c3)c2)cc1] 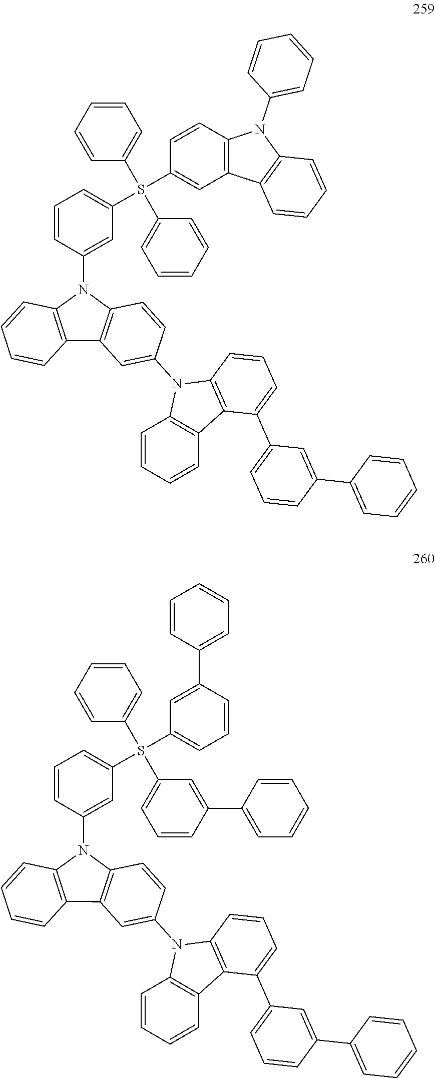 [SMILES: c1ccc(-c2cccc(-c3cccc4c3c3ccccc3n4-c3ccc4c(c3)c3ccccc3n4-c3cccc(S(c4ccccc4)(c4ccccc4)c4ccc5c(c4)c4ccccc4n5-c4ccccc4)c3)c2)cc1 ; c1ccc(-c2cccc(-c3cccc4c3c3ccccc3n4-c3ccc4c(c3)c3ccccc3n4-c3cccc(S(c4ccccc4)(c4cccc(-c5ccccc5)c4)c4cccc(-c5ccccc5)c4)c3)c2)cc1] 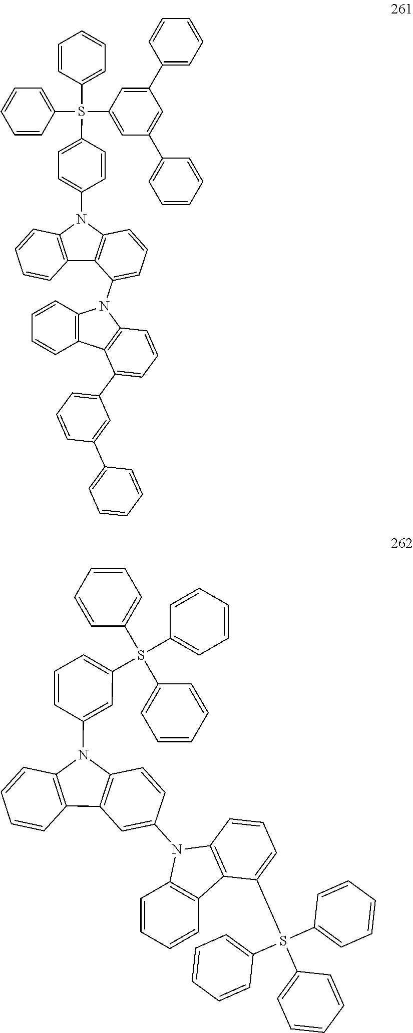 [SMILES: c1ccc(-c2cccc(-c3cccc4c3c3ccccc3n4-c3cccc4c3c3ccccc3n4-c3ccc(S(c4ccccc4)(c4ccccc4)c4cc(-c5ccccc5)cc(-c5ccccc5)c4)cc3)c2)cc1 ; c1ccc(S(c2ccccc2)(c2ccccc2)c2cccc(-n3c4ccccc4c4cc(-n5c6ccccc6c6c(S(c7ccccc7)(c7ccccc7)c7ccccc7)cccc65)ccc43)c2)cc1] 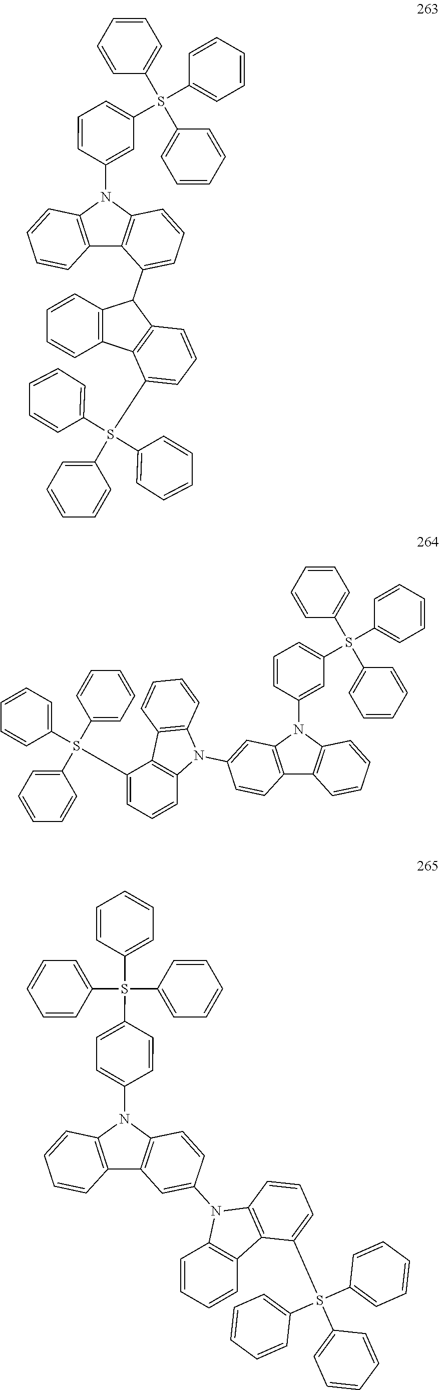 [SMILES: c1ccc(S(c2ccccc2)(c2ccccc2)c2cccc(-n3c4ccccc4c4c(C5c6ccccc6-c6c5cccc6S(c5ccccc5)(c5ccccc5)c5ccccc5)cccc43)c2)cc1 ; c1ccc(S(c2ccccc2)(c2ccccc2)c2cccc(-n3c4ccccc4c4ccc(-n5c6ccccc6c6c(S(c7ccccc7)(c7ccccc7)c7ccccc7)cccc65)cc43)c2)cc1 ; c1ccc(S(c2ccccc2)(c2ccccc2)c2ccc(-n3c4ccccc4c4cc(-n5c6ccccc6c6c(S(c7ccccc7)(c7ccccc7)c7ccccc7)cccc65)ccc43)cc2)cc1] 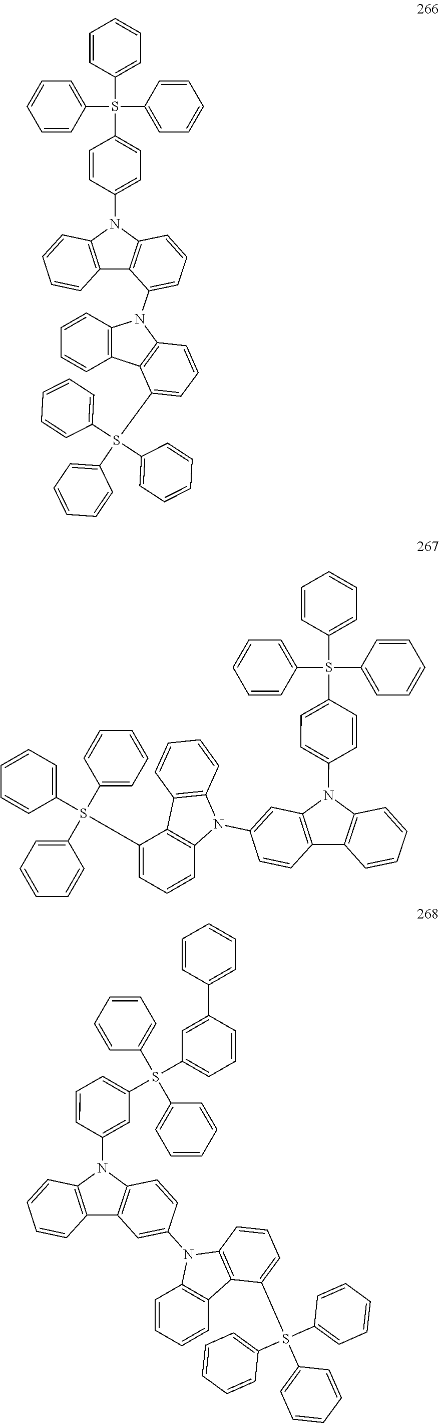 [SMILES: c1ccc(S(c2ccccc2)(c2ccccc2)c2ccc(-n3c4ccccc4c4c(-n5c6ccccc6c6c(S(c7ccccc7)(c7ccccc7)c7ccccc7)cccc65)cccc43)cc2)cc1 ; c1ccc(S(c2ccccc2)(c2ccccc2)c2ccc(-n3c4ccccc4c4ccc(-n5c6ccccc6c6c(S(c7ccccc7)(c7ccccc7)c7ccccc7)cccc65)cc43)cc2)cc1 ; c1ccc(-c2cccc(S(c3ccccc3)(c3ccccc3)c3cccc(-n4c5ccccc5c5cc(-n6c7ccccc7c7c(S(c8ccccc8)(c8ccccc8)c8ccccc8)cccc76)ccc54)c3)c2)cc1] 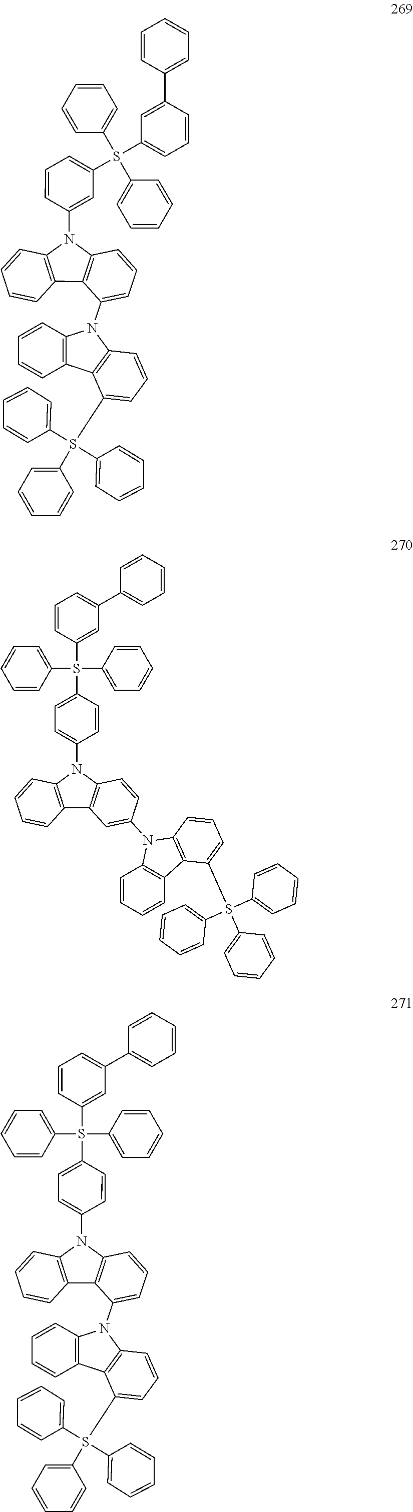 [SMILES: c1ccc(-c2cccc(S(c3ccccc3)(c3ccccc3)c3cccc(-n4c5ccccc5c5c(-n6c7ccccc7c7c(S(c8ccccc8)(c8ccccc8)c8ccccc8)cccc76)cccc54)c3)c2)cc1 ; c1ccc(-c2cccc(S(c3ccccc3)(c3ccccc3)c3ccc(-n4c5ccccc5c5cc(-n6c7ccccc7c7c(S(c8ccccc8)(c8ccccc8)c8ccccc8)cccc76)ccc54)cc3)c2)cc1 ; c1ccc(-c2cccc(S(c3ccccc3)(c3ccccc3)c3ccc(-n4c5ccccc5c5c(-n6c7ccccc7c7c(S(c8ccccc8)(c8ccccc8)c8ccccc8)cccc76)cccc54)cc3)c2)cc1] 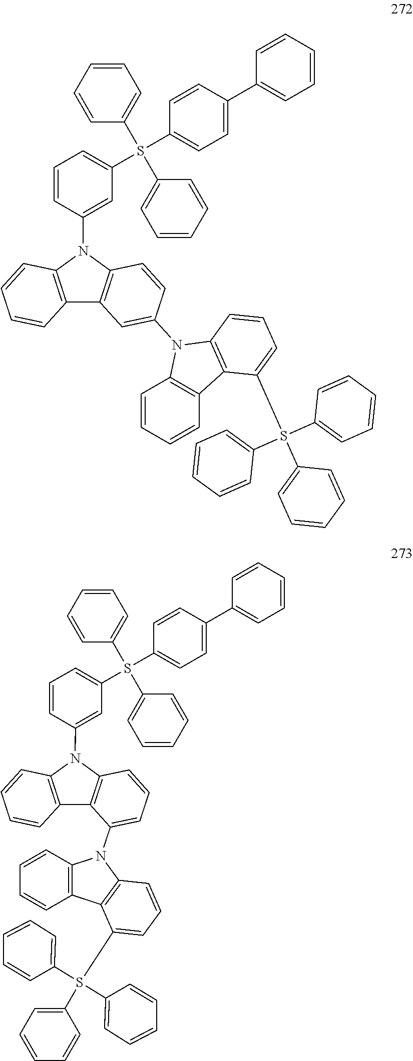 [SMILES: c1ccc(-c2ccc(S(c3ccccc3)(c3ccccc3)c3cccc(-n4c5ccccc5c5cc(-n6c7ccccc7c7c(S(c8ccccc8)(c8ccccc8)c8ccccc8)cccc76)ccc54)c3)cc2)cc1 ; c1ccc(-c2ccc(S(c3ccccc3)(c3ccccc3)c3cccc(-n4c5ccccc5c5c(-n6c7ccccc7c7c(S(c8ccccc8)(c8ccccc8)c8ccccc8)cccc76)cccc54)c3)cc2)cc1] 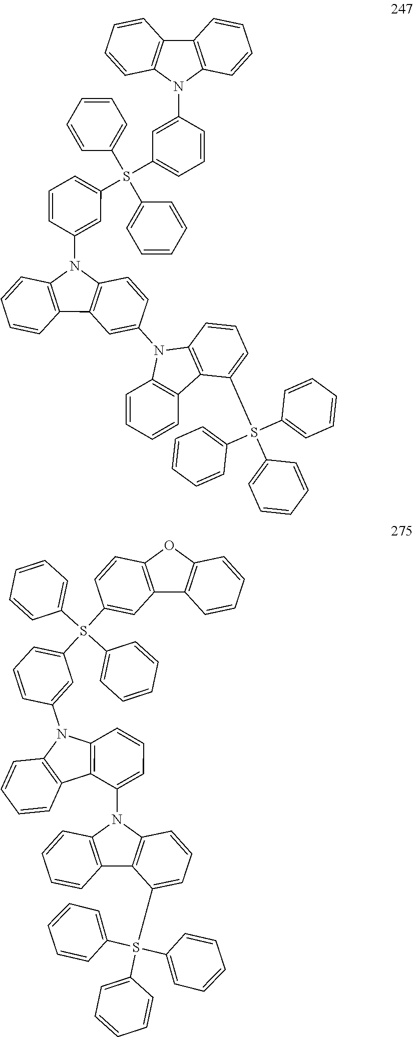 [SMILES: c1ccc(S(c2ccccc2)(c2cccc(-n3c4ccccc4c4ccccc43)c2)c2cccc(-n3c4ccccc4c4cc(-n5c6ccccc6c6c(S(c7ccccc7)(c7ccccc7)c7ccccc7)cccc65)ccc43)c2)cc1 ; c1ccc(S(c2ccccc2)(c2cccc(-n3c4ccccc4c4c(-n5c6ccccc6c6c(S(c7ccccc7)(c7ccccc7)c7ccccc7)cccc65)cccc43)c2)c2ccc3oc4ccccc4c3c2)cc1] 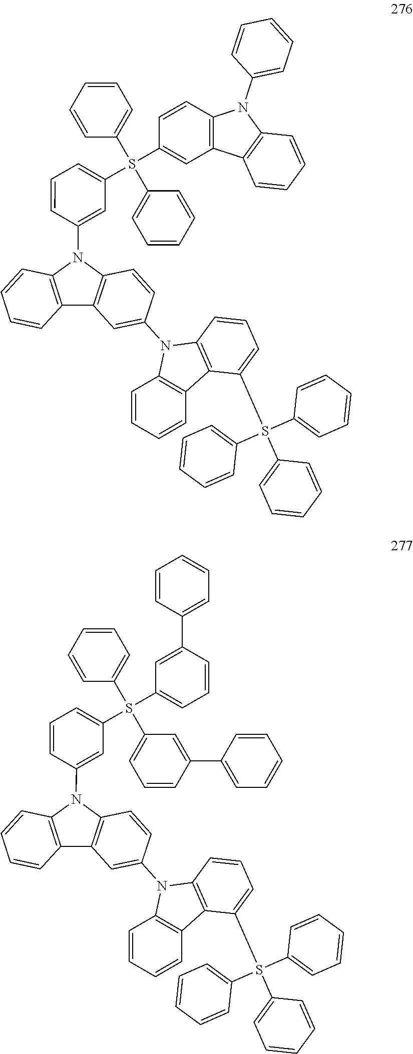 [SMILES: c1ccc(-n2c3ccccc3c3cc(S(c4ccccc4)(c4ccccc4)c4cccc(-n5c6ccccc6c6cc(-n7c8ccccc8c8c(S(c9ccccc9)(c9ccccc9)c9ccccc9)cccc87)ccc65)c4)ccc32)cc1 ; c1ccc(-c2cccc(S(c3ccccc3)(c3cccc(-c4ccccc4)c3)c3cccc(-n4c5ccccc5c5cc(-n6c7ccccc7c7c(S(c8ccccc8)(c8ccccc8)c8ccccc8)cccc76)ccc54)c3)c2)cc1] 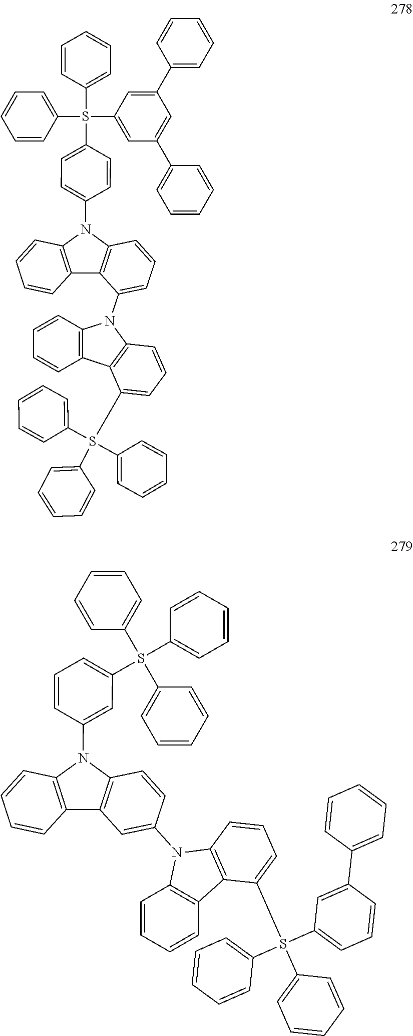 [SMILES: c1ccc(-c2cc(-c3ccccc3)cc(S(c3ccccc3)(c3ccccc3)c3ccc(-n4c5ccccc5c5c(-n6c7ccccc7c7c(S(c8ccccc8)(c8ccccc8)c8ccccc8)cccc76)cccc54)cc3)c2)cc1 ; c1ccc(-c2cccc(S(c3ccccc3)(c3ccccc3)c3cccc4c3c3ccccc3n4-c3ccc4c(c3)c3ccccc3n4-c3cccc(S(c4ccccc4)(c4ccccc4)c4ccccc4)c3)c2)cc1] 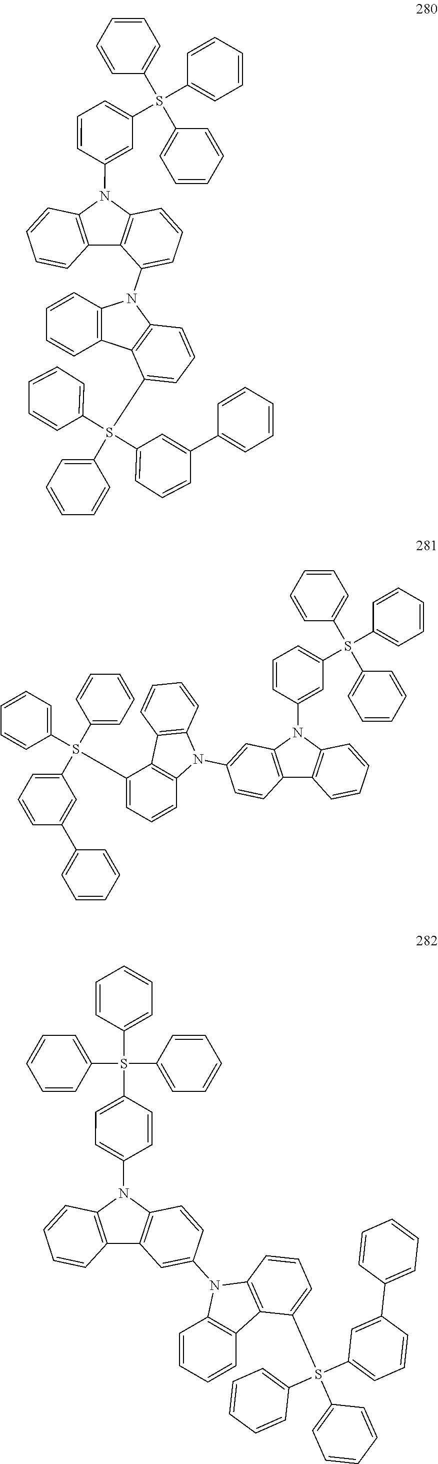 [SMILES: c1ccc(-c2cccc(S(c3ccccc3)(c3ccccc3)c3cccc4c3c3ccccc3n4-c3cccc4c3c3ccccc3n4-c3cccc(S(c4ccccc4)(c4ccccc4)c4ccccc4)c3)c2)cc1 ; c1ccc(-c2cccc(S(c3ccccc3)(c3ccccc3)c3cccc4c3c3ccccc3n4-c3ccc4c5ccccc5n(-c5cccc(S(c6ccccc6)(c6ccccc6)c6ccccc6)c5)c4c3)c2)cc1 ; c1ccc(-c2cccc(S(c3ccccc3)(c3ccccc3)c3cccc4c3c3ccccc3n4-c3ccc4c(c3)c3ccccc3n4-c3ccc(S(c4ccccc4)(c4ccccc4)c4ccccc4)cc3)c2)cc1] 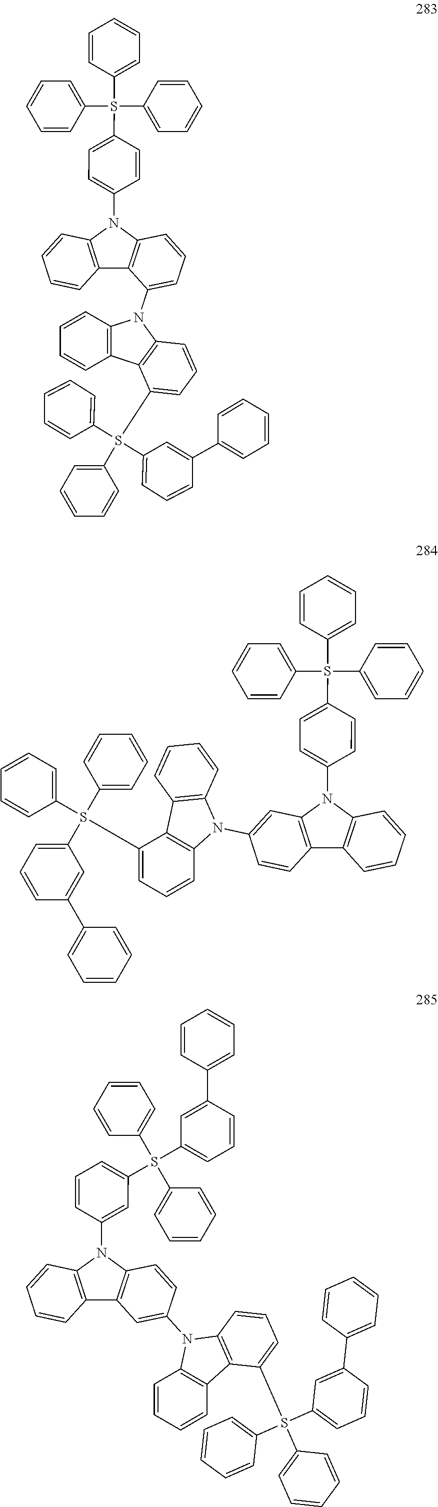 [SMILES: c1ccc(-c2cccc(S(c3ccccc3)(c3ccccc3)c3cccc4c3c3ccccc3n4-c3cccc4c3c3ccccc3n4-c3ccc(S(c4ccccc4)(c4ccccc4)c4ccccc4)cc3)c2)cc1 ; c1ccc(-c2cccc(S(c3ccccc3)(c3ccccc3)c3cccc4c3c3ccccc3n4-c3ccc4c5ccccc5n(-c5ccc(S(c6ccccc6)(c6ccccc6)c6ccccc6)cc5)c4c3)c2)cc1 ; c1ccc(-c2cccc(S(c3ccccc3)(c3ccccc3)c3cccc(-n4c5ccccc5c5cc(-n6c7ccccc7c7c(S(c8ccccc8)(c8ccccc8)c8cccc(-c9ccccc9)c8)cccc76)ccc54)c3)c2)cc1] 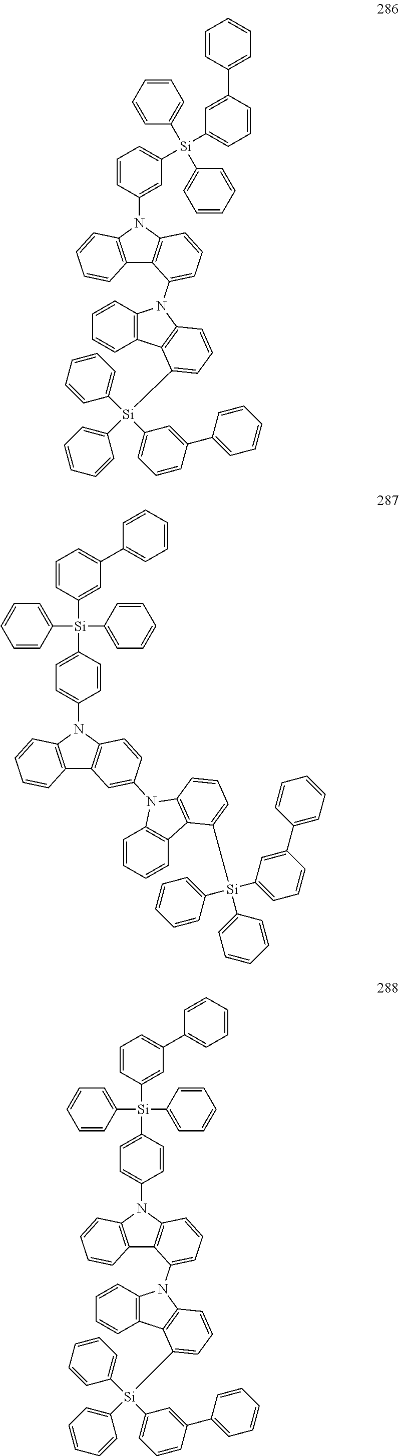 [SMILES: c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc(-c9ccccc9)c8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8cccc(-c9ccccc9)c8)cccc76)ccc54)cc3)c2)cc1 ; c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc(-c9ccccc9)c8)cccc76)cccc54)cc3)c2)cc1] 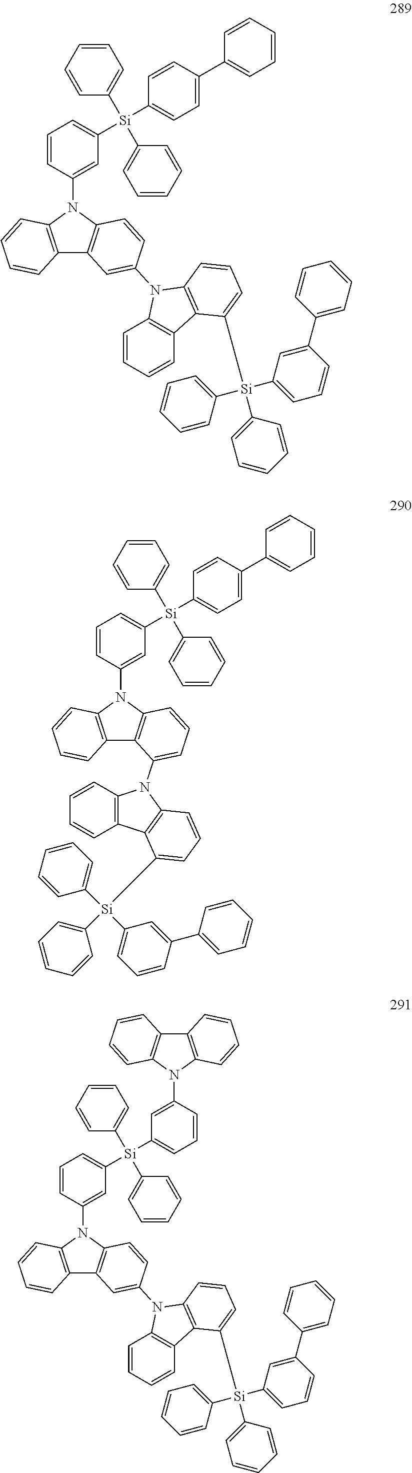 [SMILES: c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8cccc(-c9ccccc9)c8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc(-c9ccccc9)c8)cccc76)cccc54)c3)cc2)cc1 ; c1ccc(-c2cccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)c2)cc1] 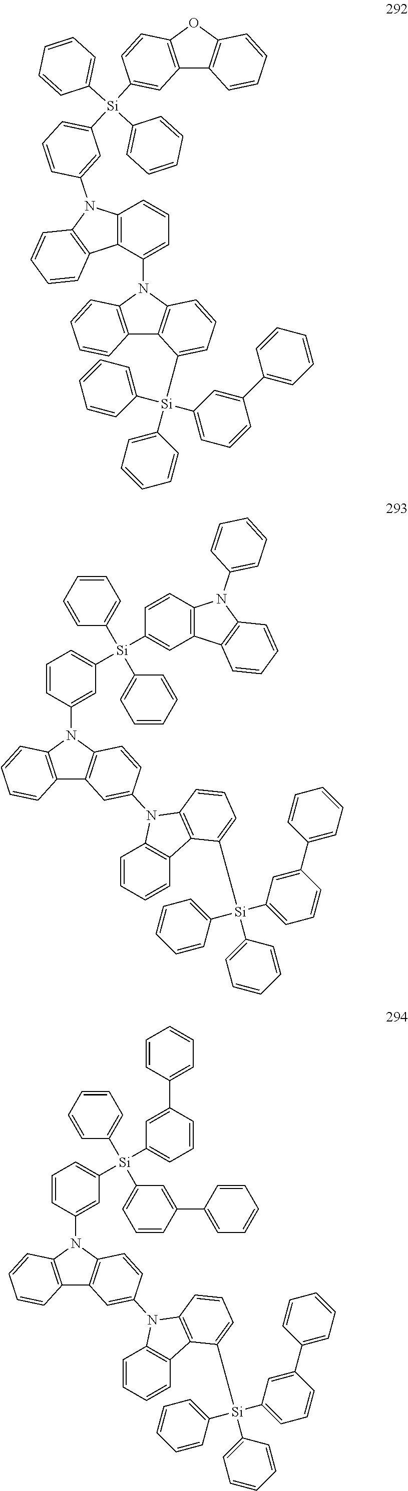 [SMILES: c1ccc(-c2cccc([Si](c3ccccc3)(c3ccccc3)c3cccc4c3c3ccccc3n4-c3cccc4c3c3ccccc3n4-c3cccc([Si](c4ccccc4)(c4ccccc4)c4ccc5oc6ccccc6c5c4)c3)c2)cc1 ; c1ccc(-c2cccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)c2)cc1 ; c1ccc(-c2cccc([Si](c3ccccc3)(c3cccc(-c4ccccc4)c3)c3cccc(-n4c5ccccc5c5cc(-n6c7ccccc7c7c([Si](c8ccccc8)(c8ccccc8)c8cccc(-c9ccccc9)c8)cccc76)ccc54)c3)c2)cc1] 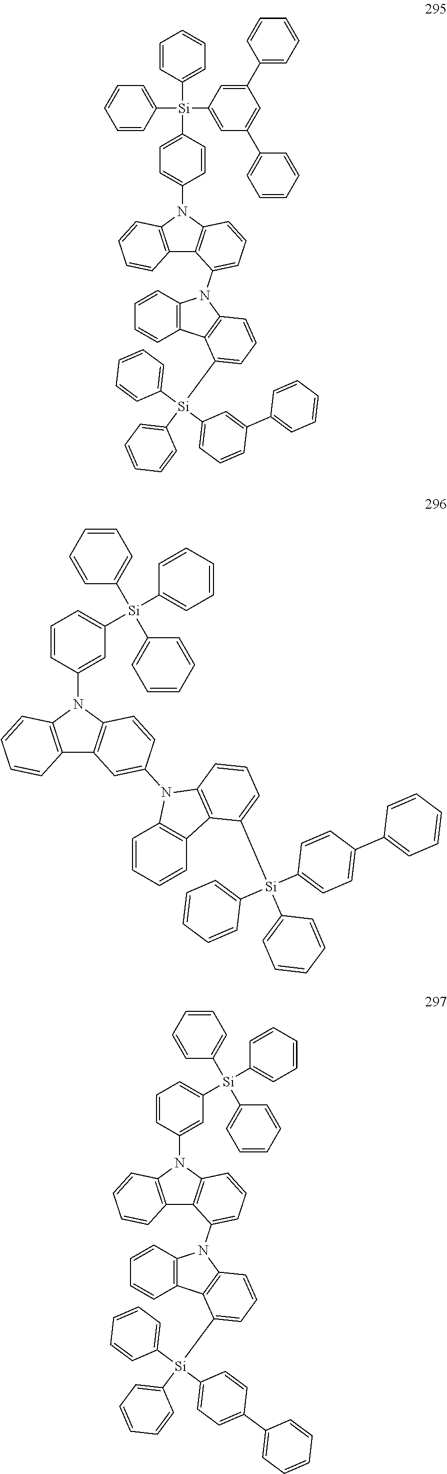 [SMILES: c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8cccc(-c9ccccc9)c8)cccc76)cccc54)cc3)c2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccccc4)c3)cc2)cc1 ; c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4ccccc4)c3)cc2)cc1] 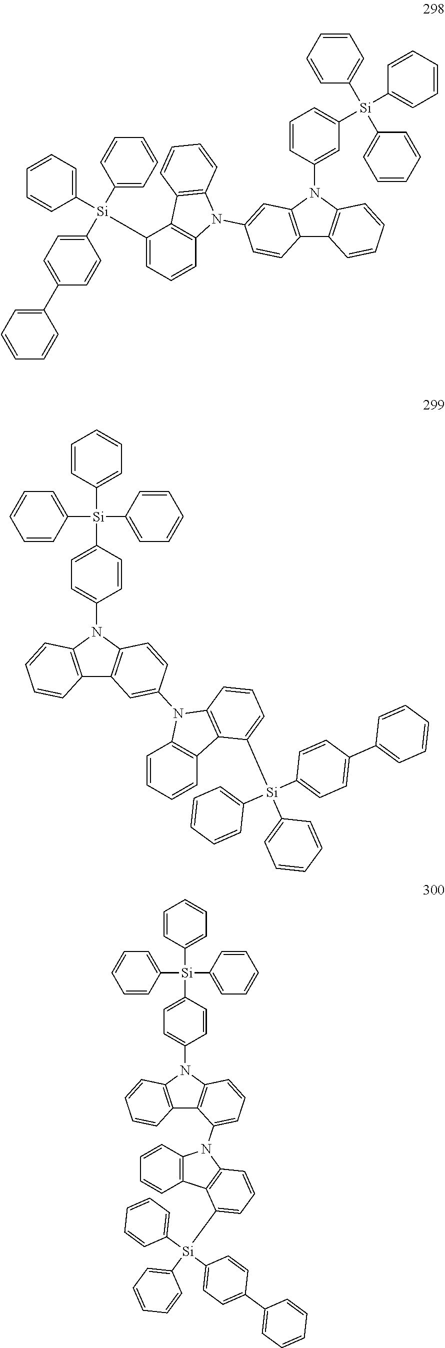 [SMILES: c1ccc(-c2ccc([Si](c3ccccc3)(c3ccccc3)c3cccc4c3c3ccccc3n4-c3ccc4c5ccccc5n(-c5cccc([Si](c6ccccc6)(c6ccccc6)c6ccccc6)c5)c4c3)cc2)cc1 ; c1ccc(-c2ccc([Si](c3ccccc3)(c3ccccc3)c3cccc4c3c3ccccc3n4-c3ccc4c(c3)c3ccccc3n4-c3ccc([Si](c4ccccc4)(c4ccccc4)c4ccccc4)cc3)cc2)cc1 ; c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4ccccc4)cc3)cc2)cc1] 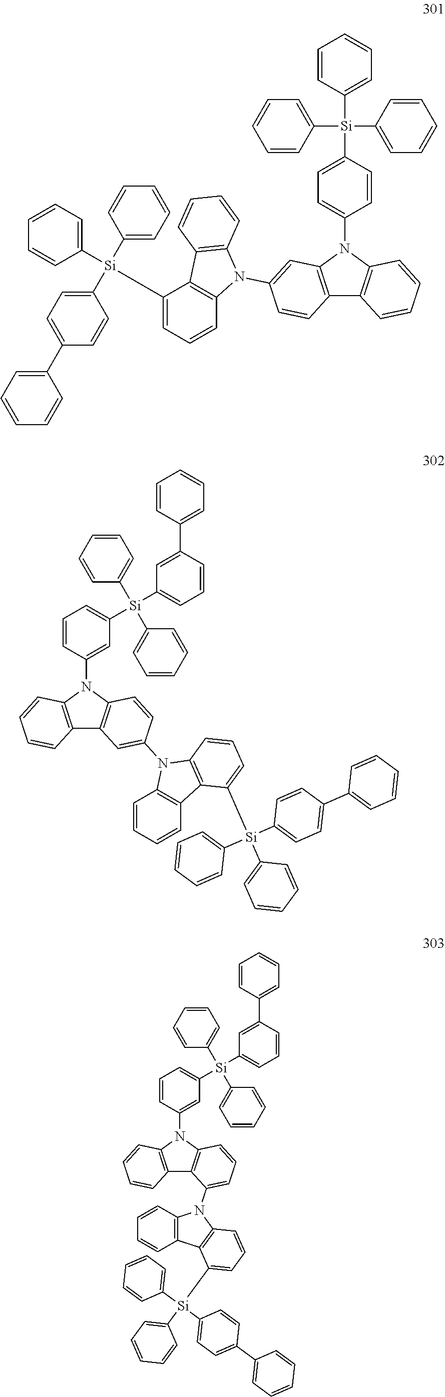 [SMILES: c1ccc(-c2ccc([Si](c3ccccc3)(c3ccccc3)c3cccc4c3c3ccccc3n4-c3ccc4c5ccccc5n(-c5ccc([Si](c6ccccc6)(c6ccccc6)c6ccccc6)cc5)c4c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-c5ccccc5)c4)c3)cc2)cc1 ; c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4cccc(-c5ccccc5)c4)c3)cc2)cc1] 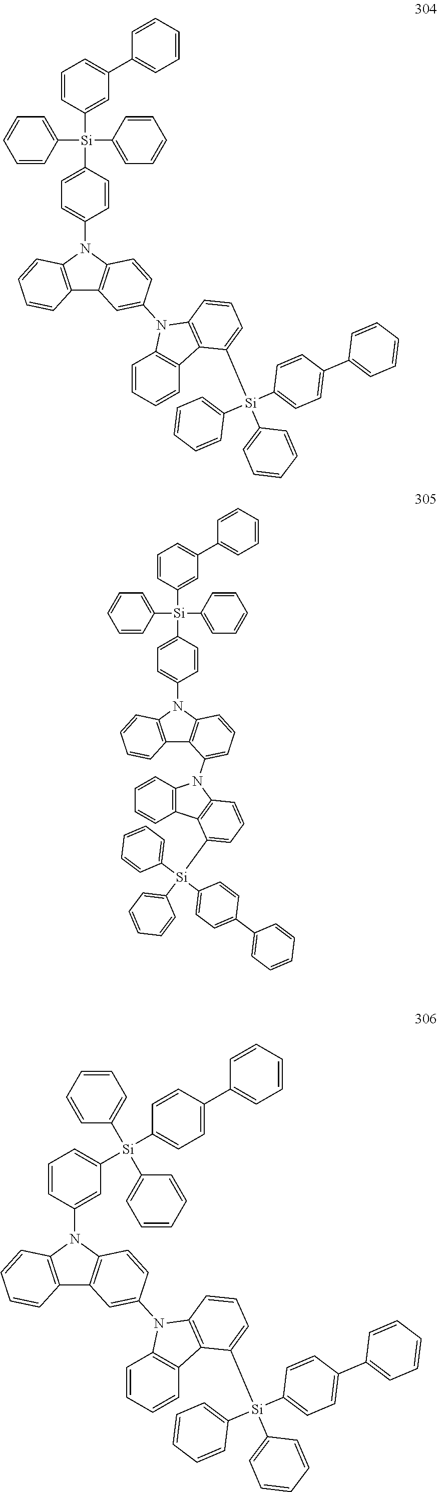 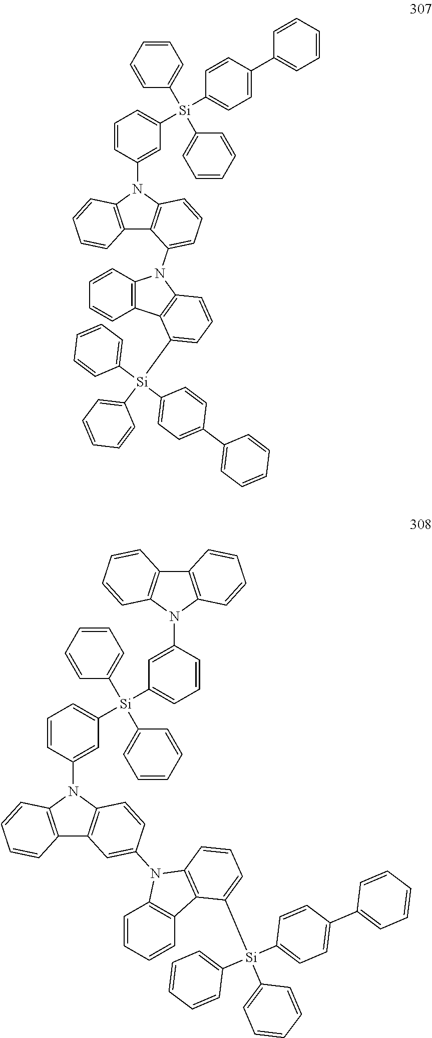 [SMILES: c1ccc(-c2ccc([Si](c3ccccc3)(c3ccccc3)c3cccc(-n4c5ccccc5c5c(-n6c7ccccc7c7c([Si](c8ccccc8)(c8ccccc8)c8ccc(-c9ccccc9)cc8)cccc76)cccc54)c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4cccc(-n5c6ccccc6c6ccccc65)c4)c3)cc2)cc1] 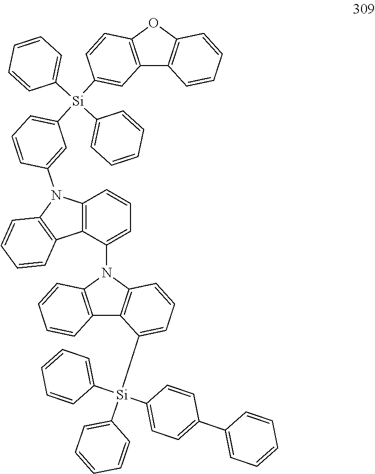 [SMILES: c1ccc(-c2ccc([Si](c3ccccc3)(c3ccccc3)c3cccc4c3c3ccccc3n4-c3cccc4c3c3ccccc3n4-c3cccc([Si](c4ccccc4)(c4ccccc4)c4ccc5oc6ccccc6c5c4)c3)cc2)cc1] 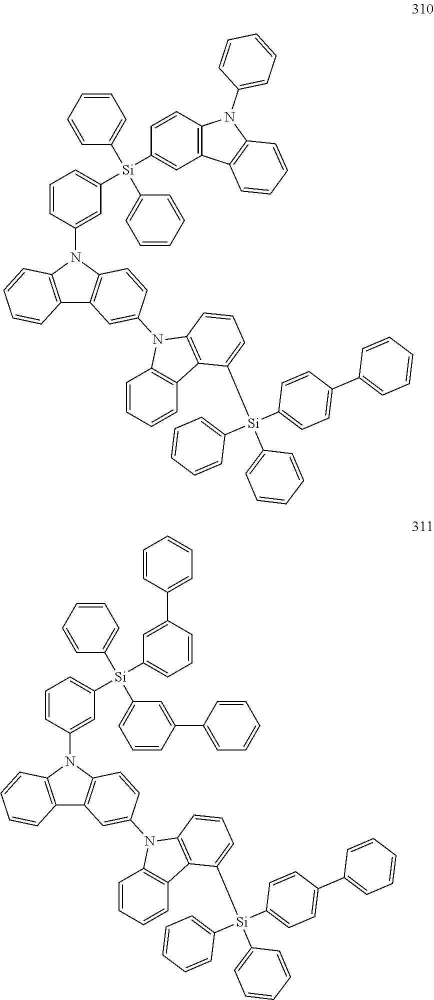 [SMILES: c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4ccccc4)c4ccc5c(c4)c4ccccc4n5-c4ccccc4)c3)cc2)cc1 ; c1ccc(-c2ccc([Si](c3ccccc3)(c3ccccc3)c3cccc4c3c3ccccc3n4-c3ccc4c(c3)c3ccccc3n4-c3cccc([Si](c4ccccc4)(c4cccc(-c5ccccc5)c4)c4cccc(-c5ccccc5)c4)c3)cc2)cc1] 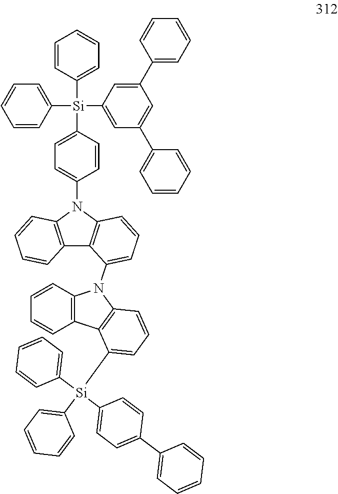 [SMILES: c1ccc(-c2ccc([Si](c3ccccc3)(c3ccccc3)c3cccc4c3c3ccccc3n4-c3cccc4c3c3ccccc3n4-c3ccc([Si](c4ccccc4)(c4ccccc4)c4cc(-c5ccccc5)cc(-c5ccccc5)c4)cc3)cc2)cc1] 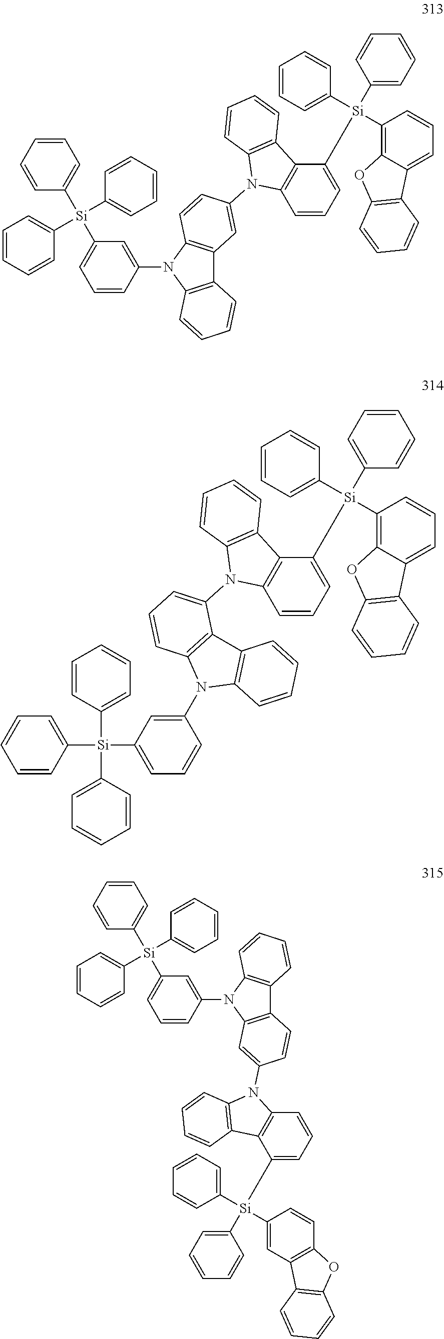 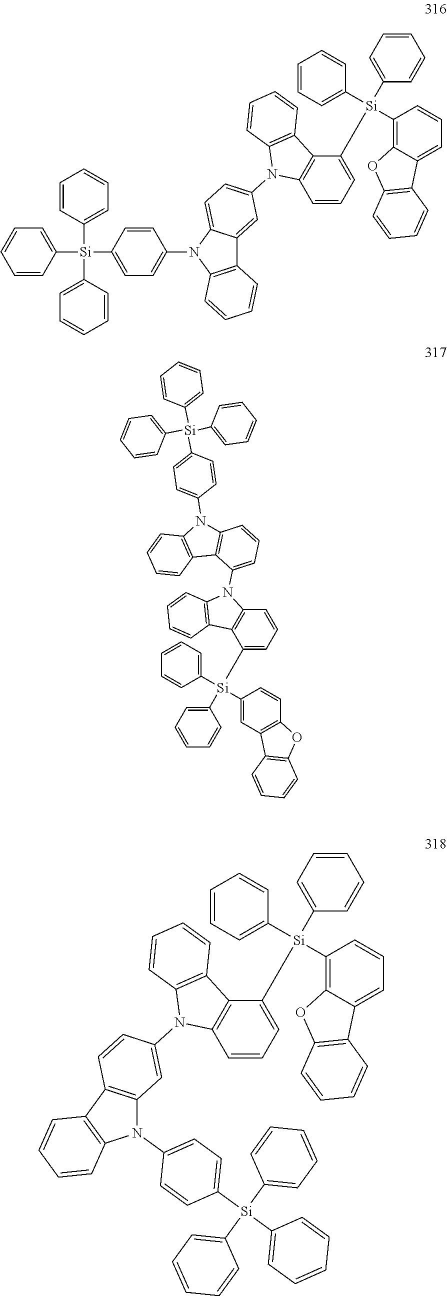 [SMILES: c1ccc([Si](c2ccccc2)(c2ccccc2)c2ccc(-n3c4ccccc4c4cc(-n5c6ccccc6c6c([Si](c7ccccc7)(c7ccccc7)c7cccc8c7oc7ccccc78)cccc65)ccc43)cc2)cc1 ; c1ccc([Si](c2ccccc2)(c2ccccc2)c2ccc(-n3c4ccccc4c4c(-n5c6ccccc6c6c([Si](c7ccccc7)(c7ccccc7)c7ccc8oc9ccccc9c8c7)cccc65)cccc43)cc2)cc1 ; c1ccc([Si](c2ccccc2)(c2ccccc2)c2ccc(-n3c4ccccc4c4ccc(-n5c6ccccc6c6c([Si](c7ccccc7)(c7ccccc7)c7cccc8c7oc7ccccc78)cccc65)cc43)cc2)cc1] 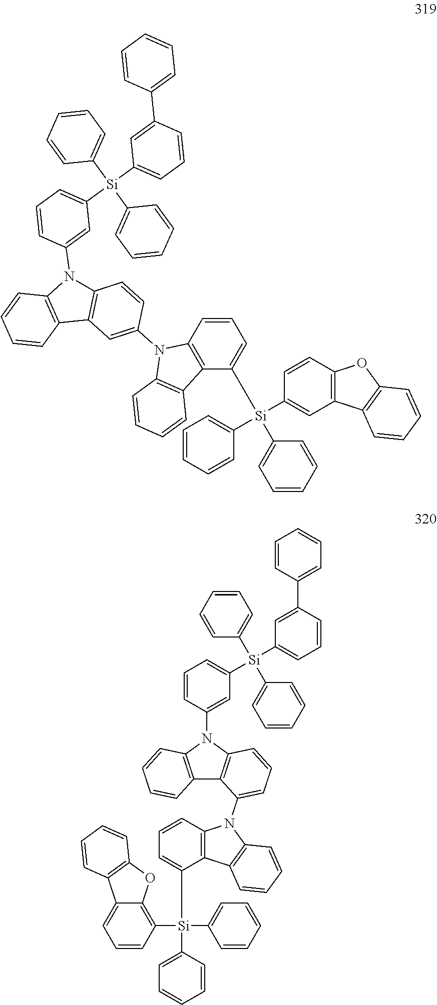 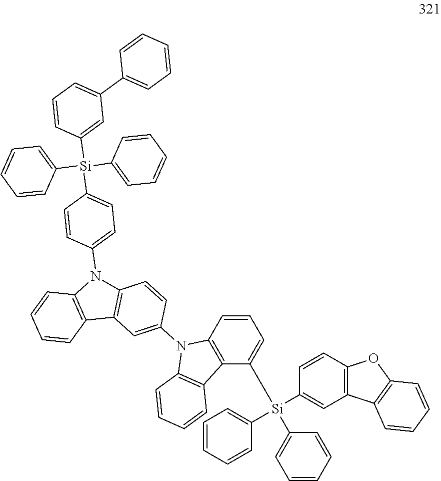 [SMILES: c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8ccc9oc%10ccccc%10c9c8)cccc76)ccc54)cc3)c2)cc1] 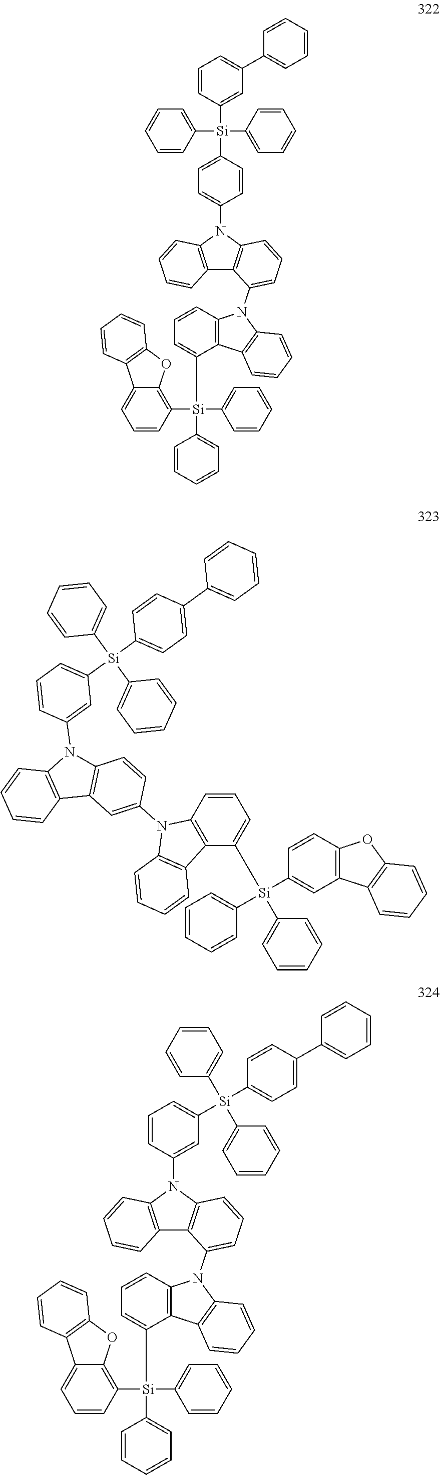 [SMILES: c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc9c8oc8ccccc89)cccc76)cccc54)cc3)c2)cc1 ; c1ccc(-c2ccc([Si](c3ccccc3)(c3ccccc3)c3cccc(-n4c5ccccc5c5cc(-n6c7ccccc7c7c([Si](c8ccccc8)(c8ccccc8)c8ccc9oc%10ccccc%10c9c8)cccc76)ccc54)c3)cc2)cc1 ; c1ccc(-c2ccc([Si](c3ccccc3)(c3ccccc3)c3cccc(-n4c5ccccc5c5c(-n6c7ccccc7c7c([Si](c8ccccc8)(c8ccccc8)c8cccc9c8oc8ccccc89)cccc76)cccc54)c3)cc2)cc1] 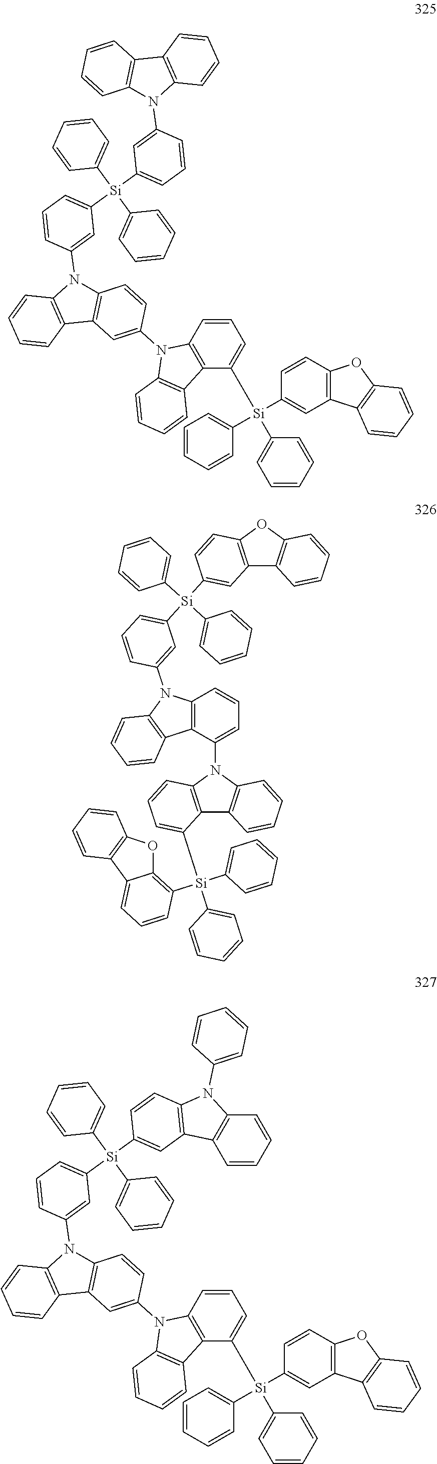 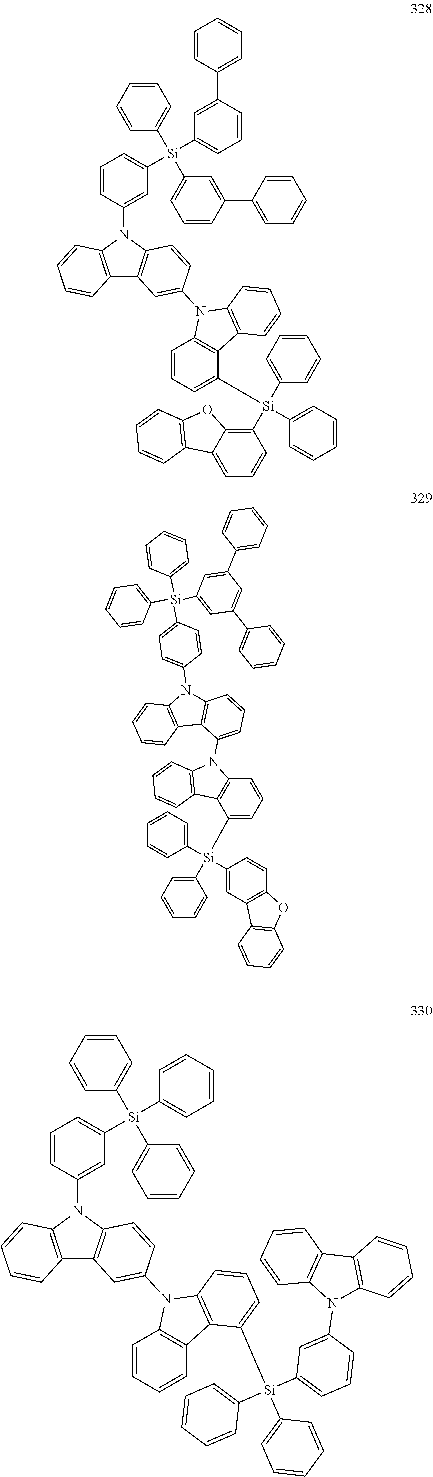 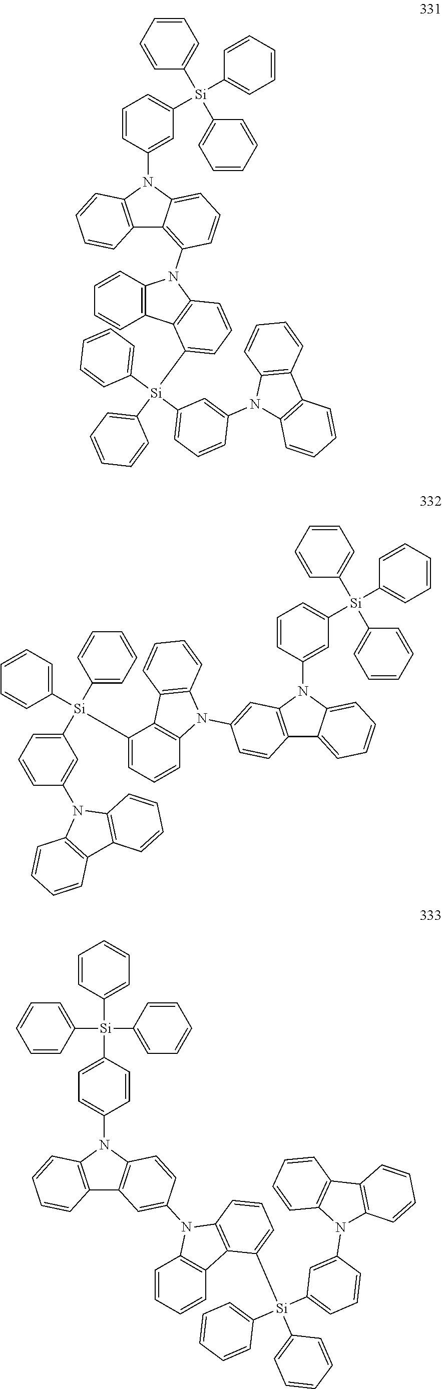 [SMILES: c1ccc([Si](c2ccccc2)(c2ccccc2)c2cccc(-n3c4ccccc4c4c(-n5c6ccccc6c6c([Si](c7ccccc7)(c7ccccc7)c7cccc(-n8c9ccccc9c9ccccc98)c7)cccc65)cccc43)c2)cc1 ; c1ccc([Si](c2ccccc2)(c2ccccc2)c2cccc(-n3c4ccccc4c4ccc(-n5c6ccccc6c6c([Si](c7ccccc7)(c7ccccc7)c7cccc(-n8c9ccccc9c9ccccc98)c7)cccc65)cc43)c2)cc1 ; c1ccc([Si](c2ccccc2)(c2ccccc2)c2ccc(-n3c4ccccc4c4cc(-n5c6ccccc6c6c([Si](c7ccccc7)(c7ccccc7)c7cccc(-n8c9ccccc9c9ccccc98)c7)cccc65)ccc43)cc2)cc1] 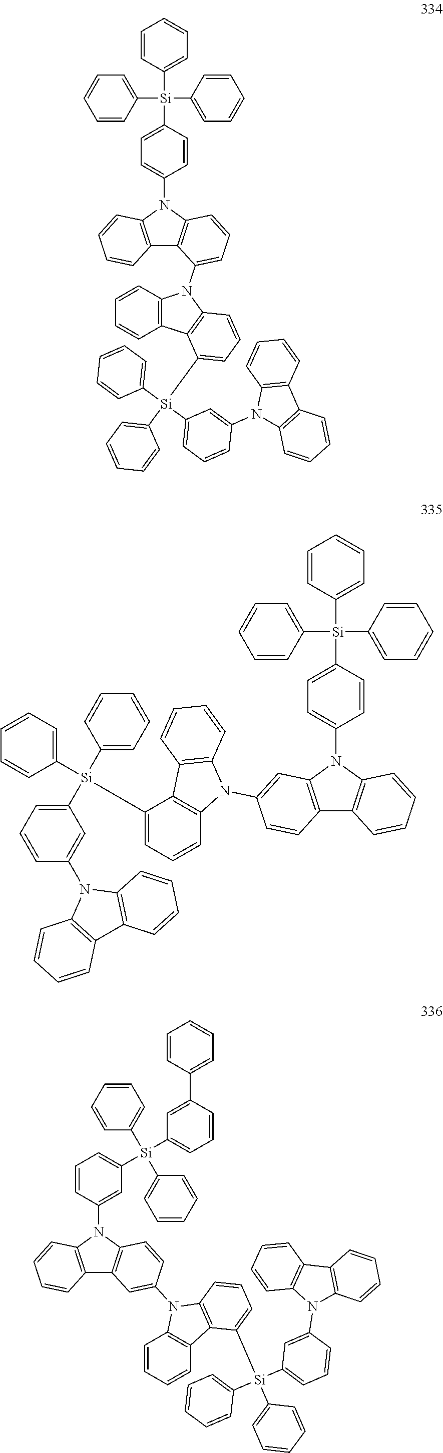 [SMILES: c1ccc([Si](c2ccccc2)(c2ccccc2)c2ccc(-n3c4ccccc4c4c(-n5c6ccccc6c6c([Si](c7ccccc7)(c7ccccc7)c7cccc(-n8c9ccccc9c9ccccc98)c7)cccc65)cccc43)cc2)cc1 ; c1ccc([Si](c2ccccc2)(c2ccccc2)c2ccc(-n3c4ccccc4c4ccc(-n5c6ccccc6c6c([Si](c7ccccc7)(c7ccccc7)c7cccc(-n8c9ccccc9c9ccccc98)c7)cccc65)cc43)cc2)cc1 ; c1ccc(-c2cccc([Si](c3ccccc3)(c3ccccc3)c3cccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)c3)c2)cc1] 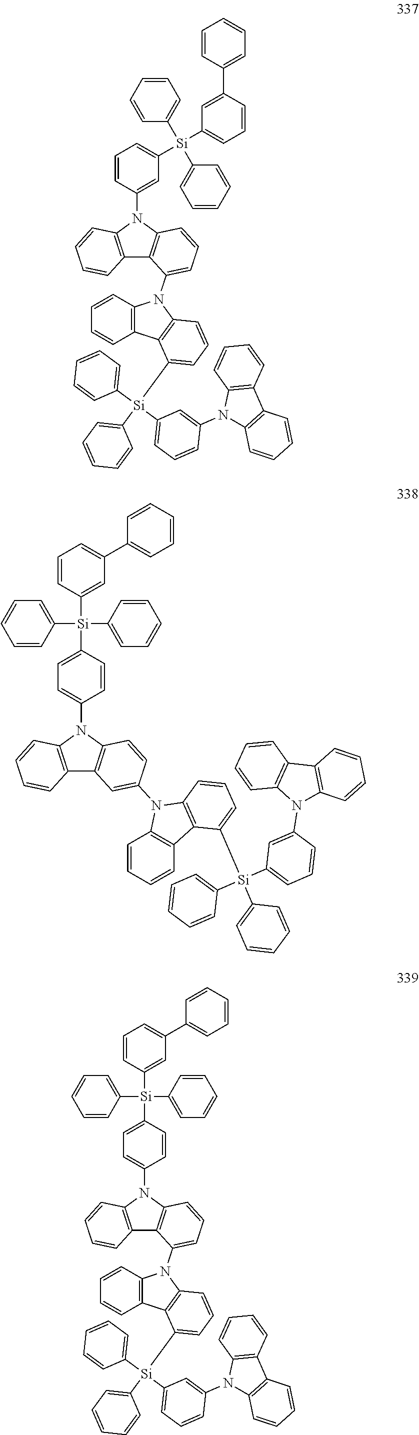 [SMILES: c1ccc(-c2cccc([Si](c3ccccc3)(c3ccccc3)c3cccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)c3)c2)cc1 ; c1ccc(-c2cccc([Si](c3ccccc3)(c3ccccc3)c3ccc(-n4c5ccccc5c5cc(-n6c7ccccc7c7c([Si](c8ccccc8)(c8ccccc8)c8cccc(-n9c%10ccccc%10c%10ccccc%109)c8)cccc76)ccc54)cc3)c2)cc1 ; c1ccc(-c2cccc([Si](c3ccccc3)(c3ccccc3)c3ccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)cc3)c2)cc1] 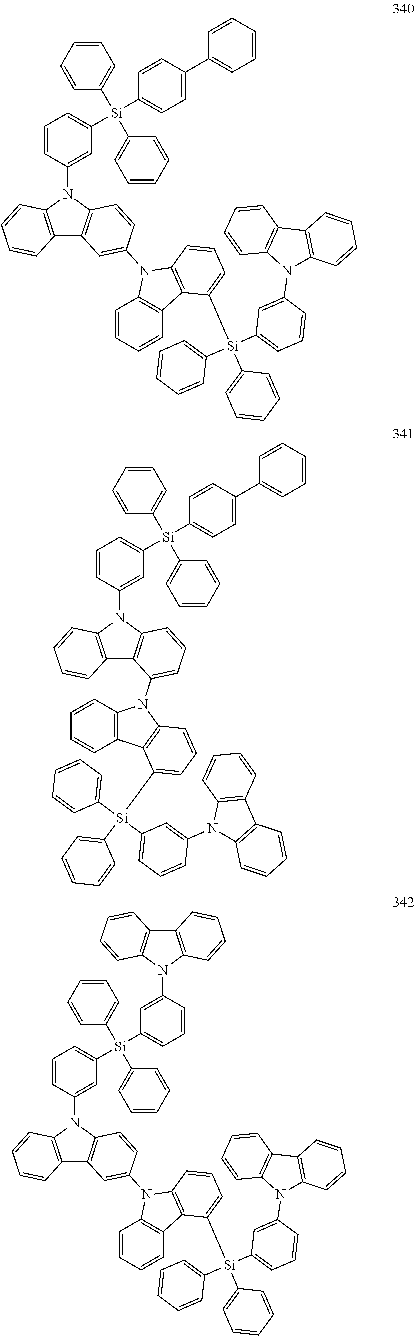 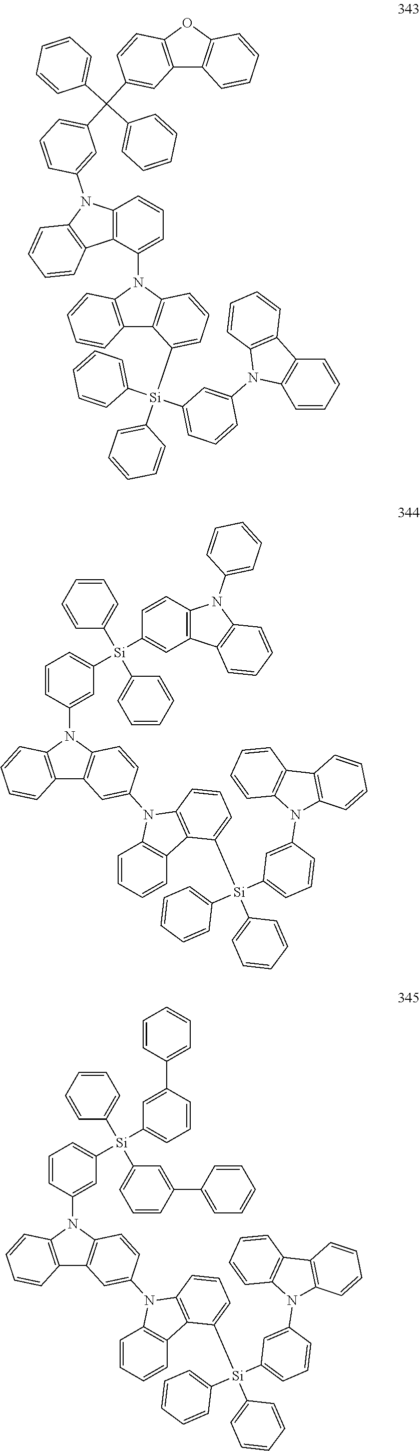 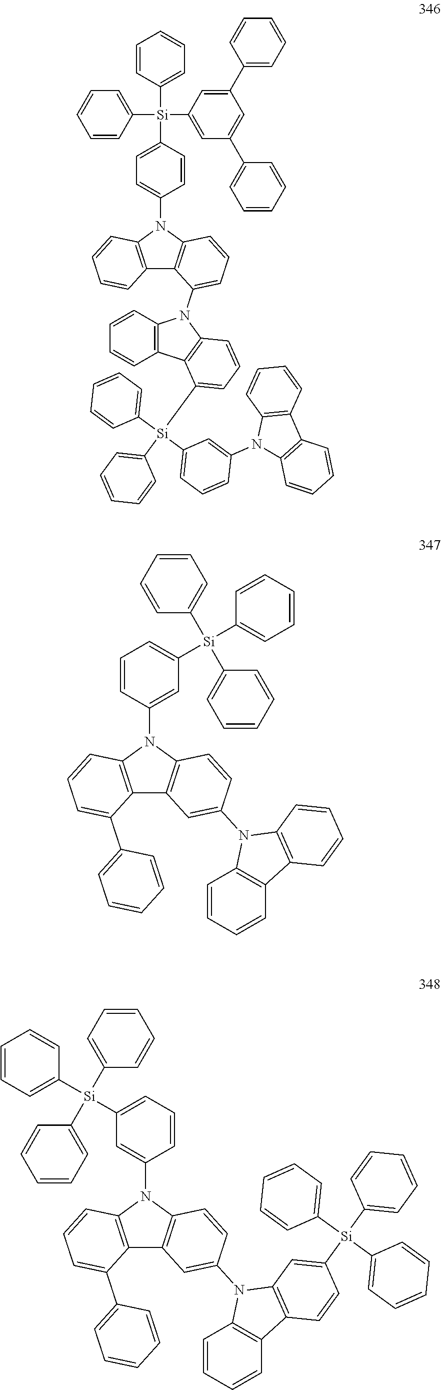 [SMILES: c1ccc(-c2cc(-c3ccccc3)cc([Si](c3ccccc3)(c3ccccc3)c3ccc(-n4c5ccccc5c5c(-n6c7ccccc7c7c([Si](c8ccccc8)(c8ccccc8)c8cccc(-n9c%10ccccc%10c%10ccccc%109)c8)cccc76)cccc54)cc3)c2)cc1 ; c1ccc(-c2cccc3c2c2cc(-n4c5ccccc5c5ccccc54)ccc2n3-c2cccc([Si](c3ccccc3)(c3ccccc3)c3ccccc3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccccc3)c2)cc1] 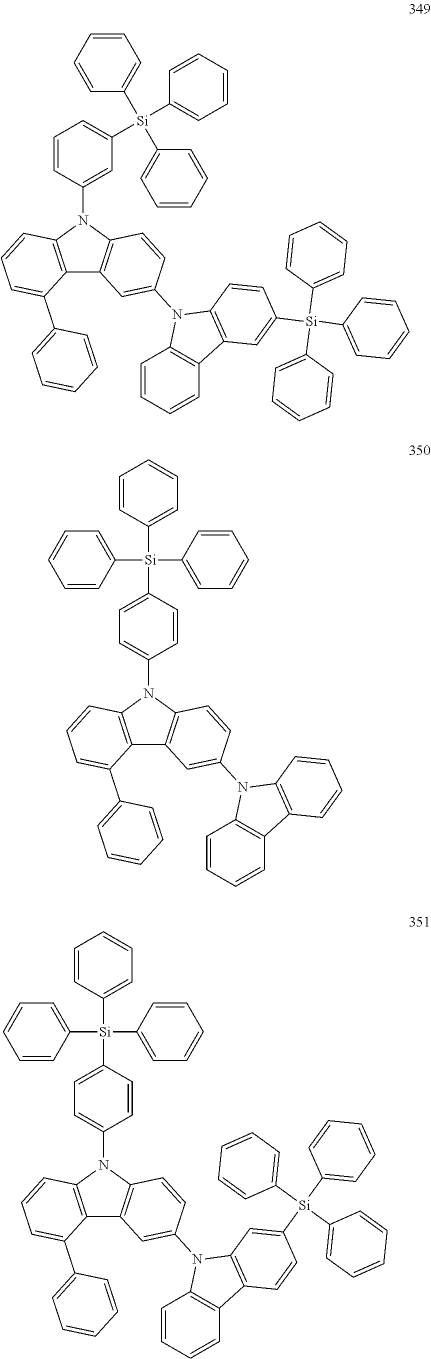 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3ccccc3)c2)cc1 ; c1ccc(-c2cccc3c2c2cc(-n4c5ccccc5c5ccccc54)ccc2n3-c2ccc([Si](c3ccccc3)(c3ccccc3)c3ccccc3)cc2)cc1 ; c1ccc(-c2cccc3c2c2cc(-n4c5ccccc5c5ccc([Si](c6ccccc6)(c6ccccc6)c6ccccc6)cc54)ccc2n3-c2ccc([Si](c3ccccc3)(c3ccccc3)c3ccccc3)cc2)cc1] 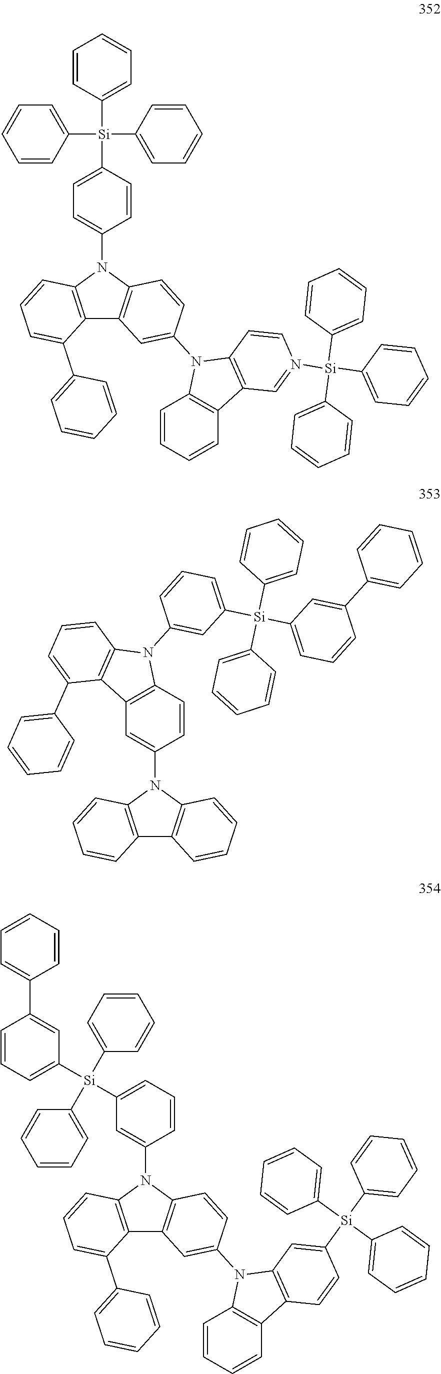 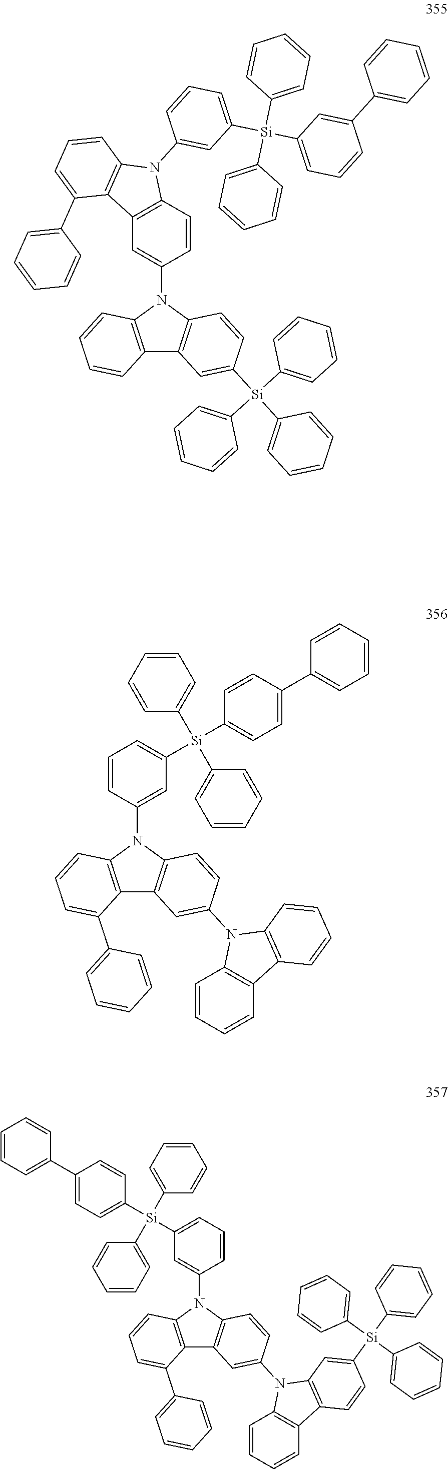 [SMILES: c1ccc(-c2cccc([Si](c3ccccc3)(c3ccccc3)c3cccc(-n4c5ccc(-n6c7ccccc7c7cc([Si](c8ccccc8)(c8ccccc8)c8ccccc8)ccc76)cc5c5c(-c6ccccc6)cccc54)c3)c2)cc1 ; c1ccc(-c2ccc([Si](c3ccccc3)(c3ccccc3)c3cccc(-n4c5ccc(-n6c7ccccc7c7ccccc76)cc5c5c(-c6ccccc6)cccc54)c3)cc2)cc1 ; c1ccc(-c2ccc([Si](c3ccccc3)(c3ccccc3)c3cccc(-n4c5ccc(-n6c7ccccc7c7ccc([Si](c8ccccc8)(c8ccccc8)c8ccccc8)cc76)cc5c5c(-c6ccccc6)cccc54)c3)cc2)cc1] 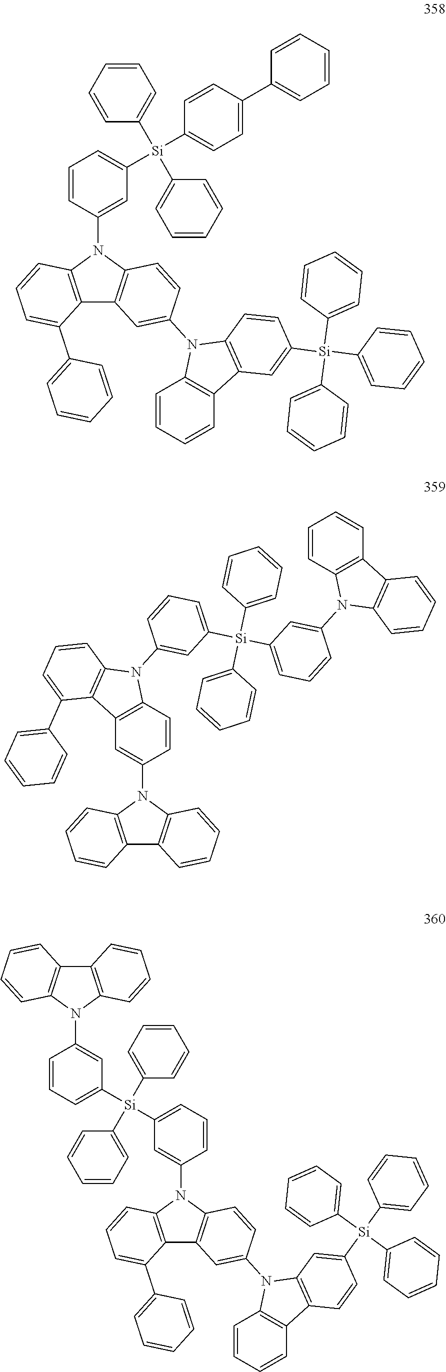 [SMILES: c1ccc(-c2ccc([Si](c3ccccc3)(c3ccccc3)c3cccc(-n4c5ccc(-n6c7ccccc7c7cc([Si](c8ccccc8)(c8ccccc8)c8ccccc8)ccc76)cc5c5c(-c6ccccc6)cccc54)c3)cc2)cc1 ; c1ccc(-c2cccc3c2c2cc(-n4c5ccccc5c5ccccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3cccc(-n4c5ccccc5c5ccccc54)c3)c2)cc1] 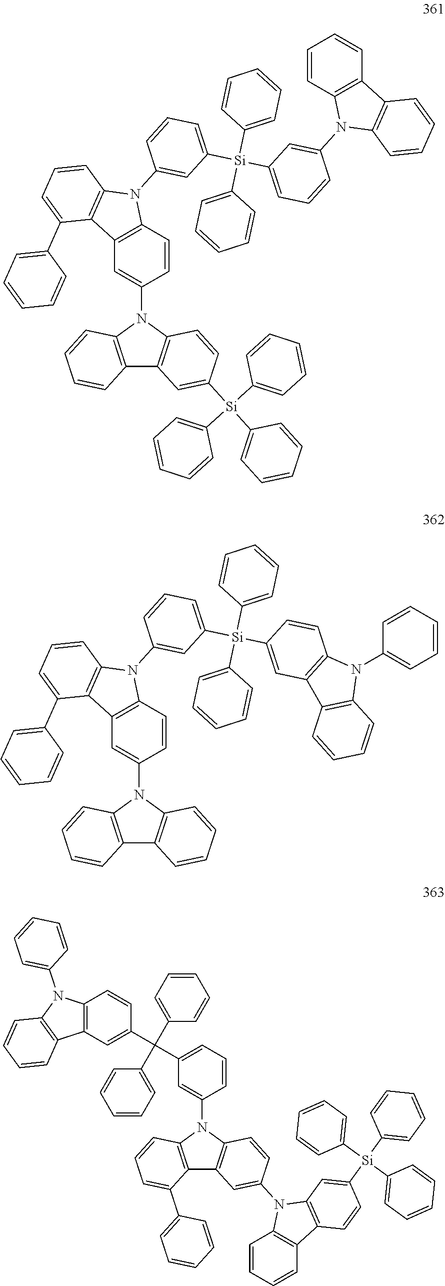 [SMILES: c1ccc(-c2cccc3c2c2cc(-n4c5ccccc5c5cc([Si](c6ccccc6)(c6ccccc6)c6ccccc6)ccc54)ccc2n3-c2cccc([Si](c3ccccc3)(c3ccccc3)c3cccc(-n4c5ccccc5c5ccccc54)c3)c2)cc1 ; c1ccc(-c2cccc3c2c2cc(-n4c5ccccc5c5ccccc54)ccc2n3-c2cccc([Si](c3ccccc3)(c3ccccc3)c3ccc4c(c3)c3ccccc3n4-c3ccccc3)c2)cc1 ; c1ccc(-c2cccc3c2c2cc(-n4c5ccccc5c5ccc([Si](c6ccccc6)(c6ccccc6)c6ccccc6)cc54)ccc2n3-c2cccc(C(c3ccccc3)(c3ccccc3)c3ccc4c(c3)c3ccccc3n4-c3ccccc3)c2)cc1] 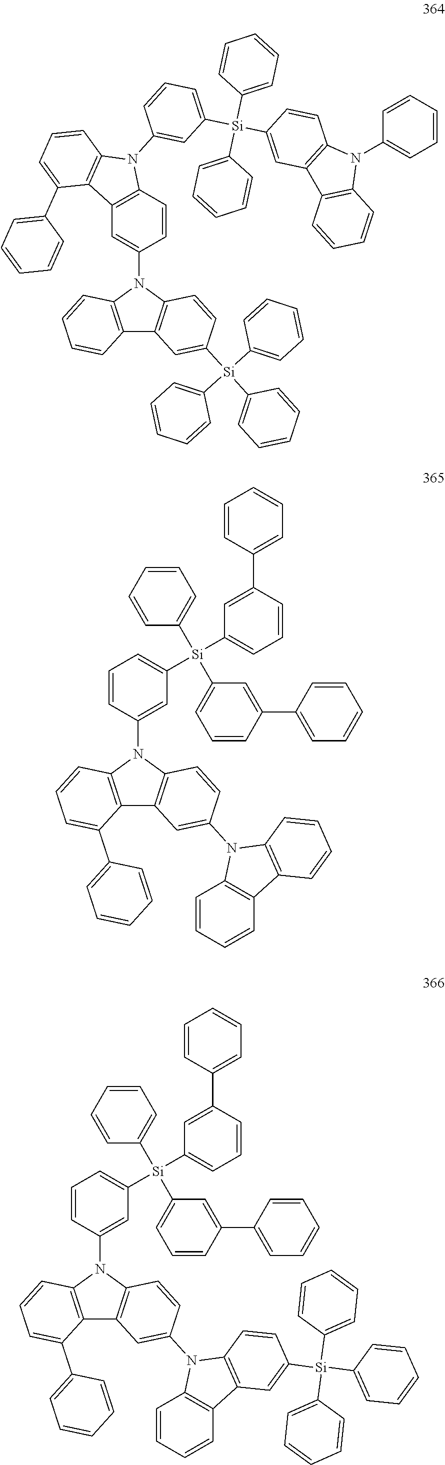 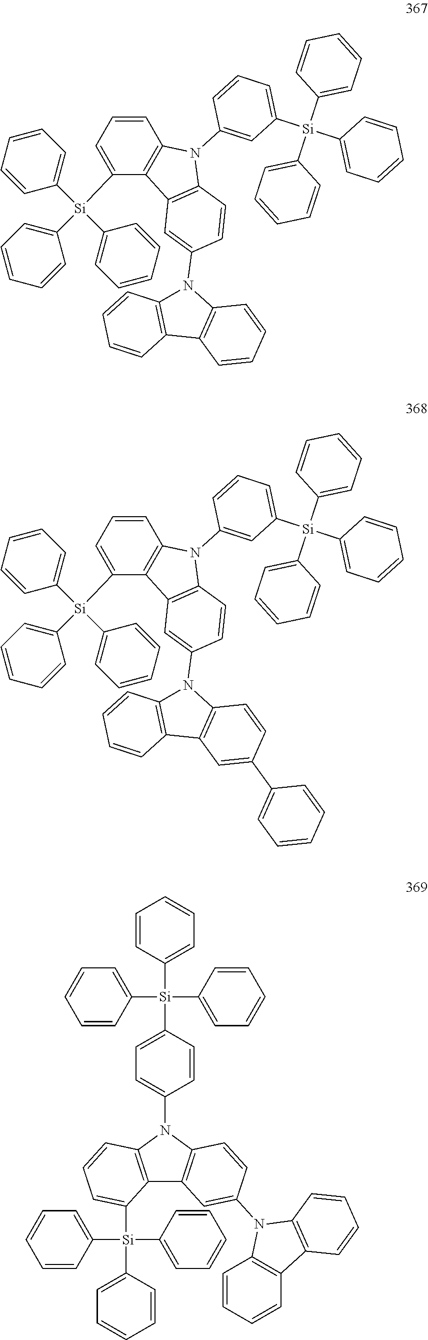 [SMILES: c1ccc([Si](c2ccccc2)(c2ccccc2)c2cccc(-n3c4ccc(-n5c6ccccc6c6ccccc65)cc4c4c([Si](c5ccccc5)(c5ccccc5)c5ccccc5)cccc43)c2)cc1 ; c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)cc1 ; c1ccc([Si](c2ccccc2)(c2ccccc2)c2ccc(-n3c4ccc(-n5c6ccccc6c6ccccc65)cc4c4c([Si](c5ccccc5)(c5ccccc5)c5ccccc5)cccc43)cc2)cc1] 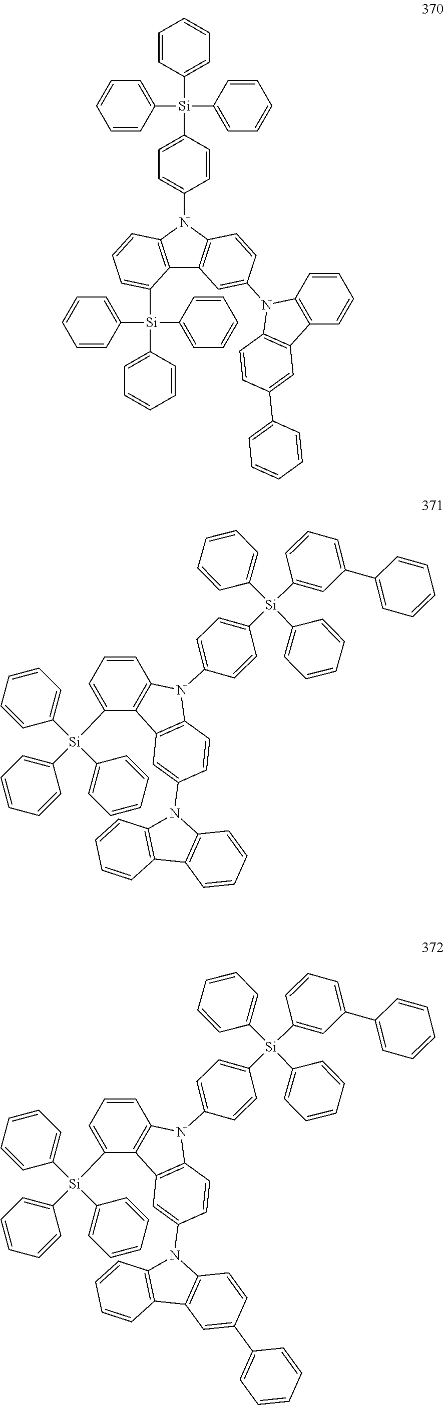 [SMILES: c1ccc(-c2ccc3c(c2)c2ccccc2n3-c2ccc3c(c2)c2c([Si](c4ccccc4)(c4ccccc4)c4ccccc4)cccc2n3-c2ccc([Si](c3ccccc3)(c3ccccc3)c3ccccc3)cc2)cc1 ; c1ccc(-c2cccc([Si](c3ccccc3)(c3ccccc3)c3ccc(-n4c5ccc(-n6c7ccccc7c7ccccc76)cc5c5c([Si](c6ccccc6)(c6ccccc6)c6ccccc6)cccc54)cc3)c2)cc1 ; c1ccc(-c2cccc([Si](c3ccccc3)(c3ccccc3)c3ccc(-n4c5ccc(-n6c7ccccc7c7cc(-c8ccccc8)ccc76)cc5c5c([Si](c6ccccc6)(c6ccccc6)c6ccccc6)cccc54)cc3)c2)cc1] 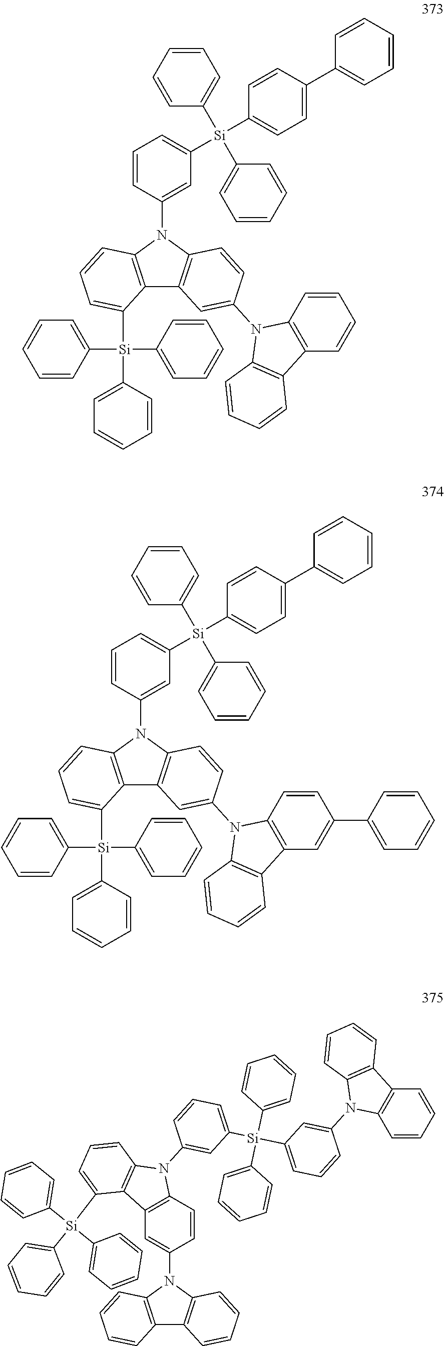 [SMILES: c1ccc(-c2ccc([Si](c3ccccc3)(c3ccccc3)c3cccc(-n4c5ccc(-n6c7ccccc7c7ccccc76)cc5c5c([Si](c6ccccc6)(c6ccccc6)c6ccccc6)cccc54)c3)cc2)cc1 ; c1ccc(-c2ccc([Si](c3ccccc3)(c3ccccc3)c3cccc(-n4c5ccc(-n6c7ccccc7c7cc(-c8ccccc8)ccc76)cc5c5c([Si](c6ccccc6)(c6ccccc6)c6ccccc6)cccc54)c3)cc2)cc1 ; c1ccc([Si](c2ccccc2)(c2cccc(-n3c4ccccc4c4ccccc43)c2)c2cccc(-n3c4ccc(-n5c6ccccc6c6ccccc65)cc4c4c([Si](c5ccccc5)(c5ccccc5)c5ccccc5)cccc43)c2)cc1] 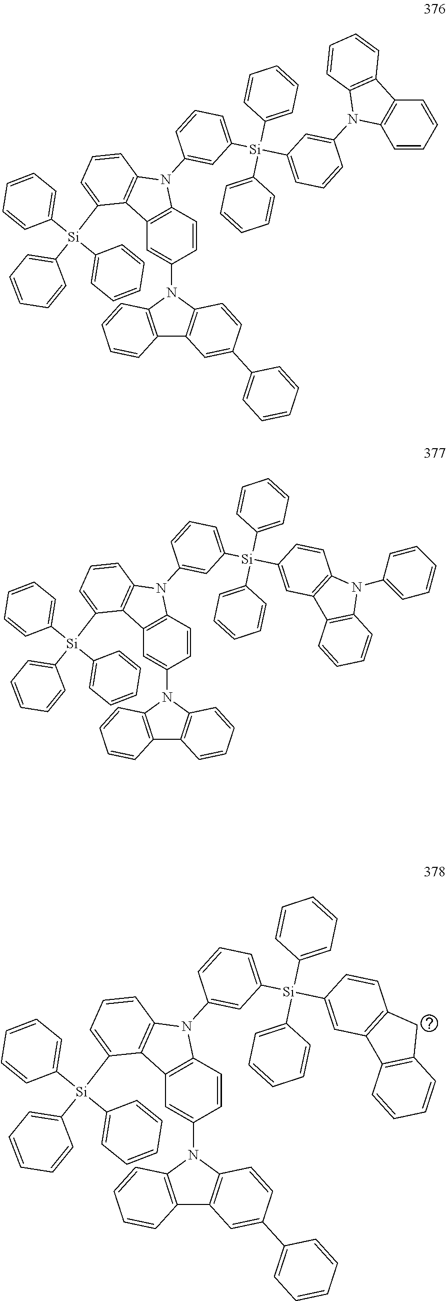 [SMILES: c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3cccc(-n4c5ccccc5c5ccccc54)c3)c2)cc1 ; c1ccc(-n2c3ccccc3c3cc([Si](c4ccccc4)(c4ccccc4)c4cccc(-n5c6ccc(-n7c8ccccc8c8ccccc87)cc6c6c([Si](c7ccccc7)(c7ccccc7)c7ccccc7)cccc65)c4)ccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4c(c3)-c3ccccc3C4)c2)cc1] 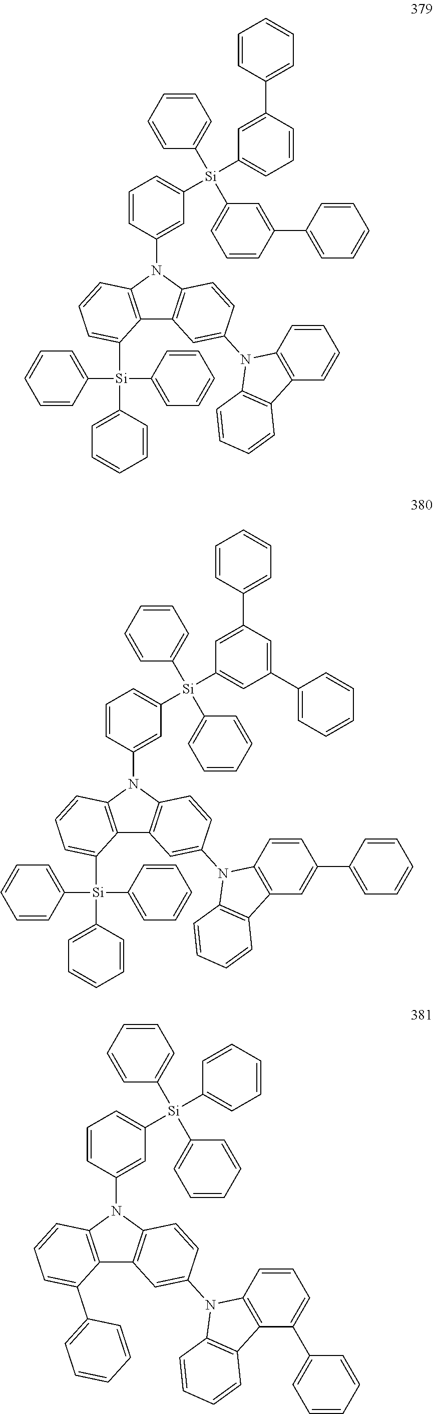 [SMILES: c1ccc(-c2cccc([Si](c3ccccc3)(c3cccc(-c4ccccc4)c3)c3cccc(-n4c5ccc(-n6c7ccccc7c7ccccc76)cc5c5c([Si](c6ccccc6)(c6ccccc6)c6ccccc6)cccc54)c3)c2)cc1 ; c1ccc(-c2cc(-c3ccccc3)cc([Si](c3ccccc3)(c3ccccc3)c3cccc(-n4c5ccc(-n6c7ccccc7c7cc(-c8ccccc8)ccc76)cc5c5c([Si](c6ccccc6)(c6ccccc6)c6ccccc6)cccc54)c3)c2)cc1 ; c1ccc(-c2cccc3c2c2ccccc2n3-c2ccc3c(c2)c2c(-c4ccccc4)cccc2n3-c2cccc([Si](c3ccccc3)(c3ccccc3)c3ccccc3)c2)cc1] 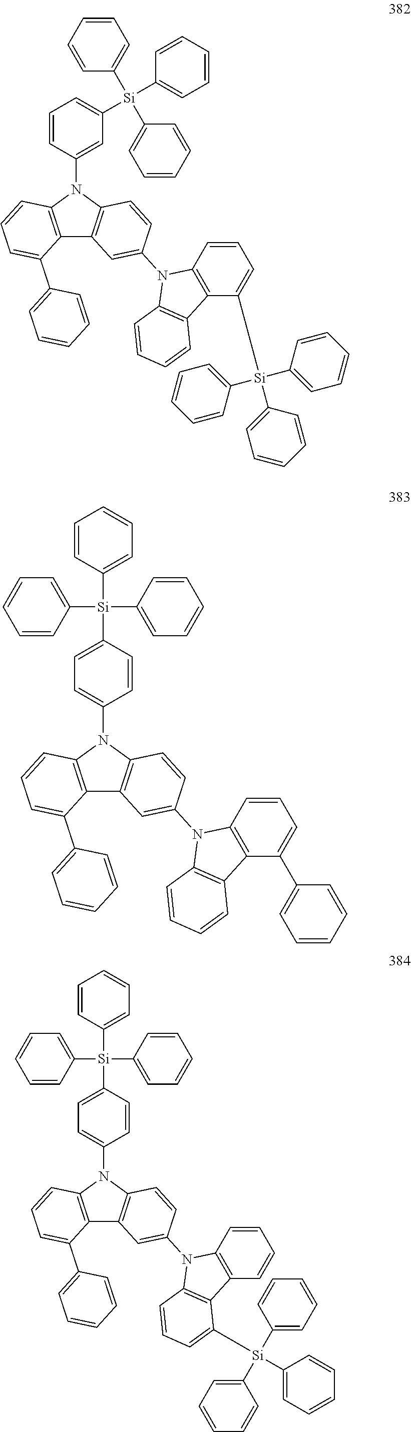 [SMILES: c1ccc(-c2cccc3c2c2cc(-n4c5ccccc5c5c([Si](c6ccccc6)(c6ccccc6)c6ccccc6)cccc54)ccc2n3-c2cccc([Si](c3ccccc3)(c3ccccc3)c3ccccc3)c2)cc1 ; c1ccc(-c2cccc3c2c2ccccc2n3-c2ccc3c(c2)c2c(-c4ccccc4)cccc2n3-c2ccc([Si](c3ccccc3)(c3ccccc3)c3ccccc3)cc2)cc1 ; c1ccc(-c2cccc3c2c2cc(-n4c5ccccc5c5c([Si](c6ccccc6)(c6ccccc6)c6ccccc6)cccc54)ccc2n3-c2ccc([Si](c3ccccc3)(c3ccccc3)c3ccccc3)cc2)cc1] 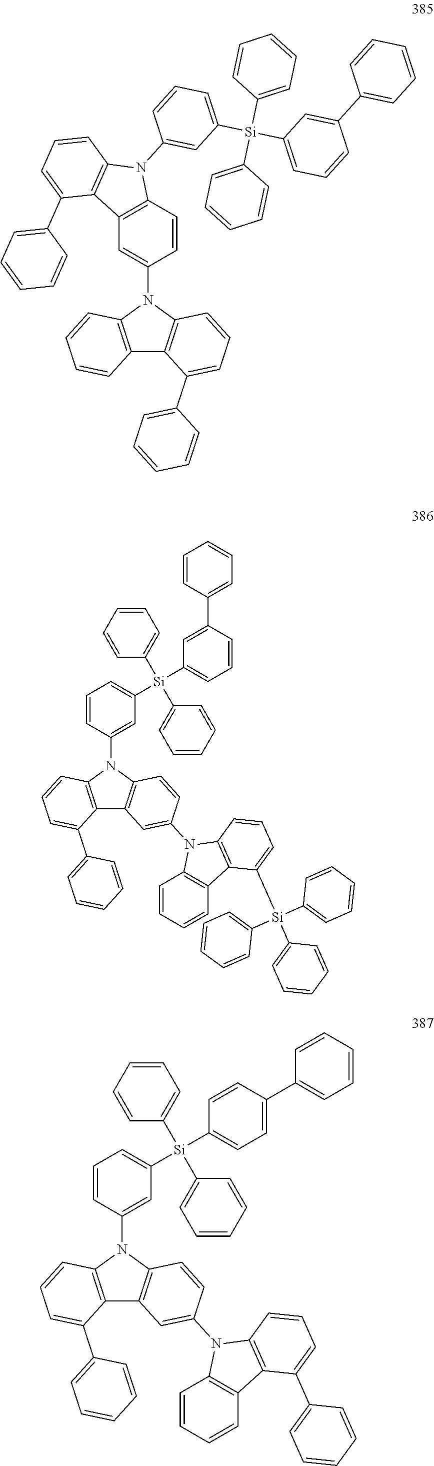 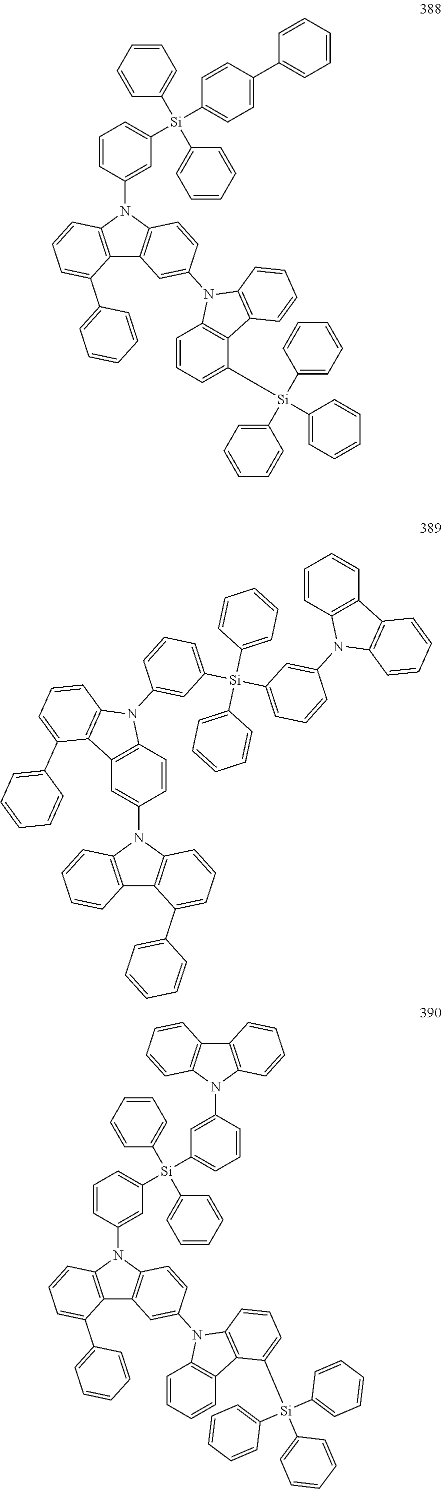 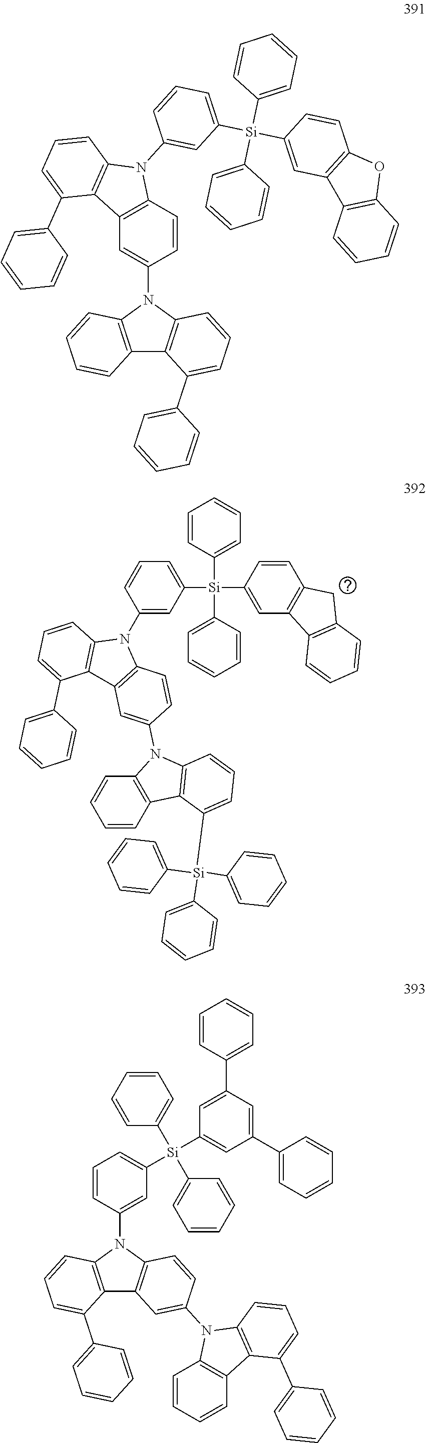 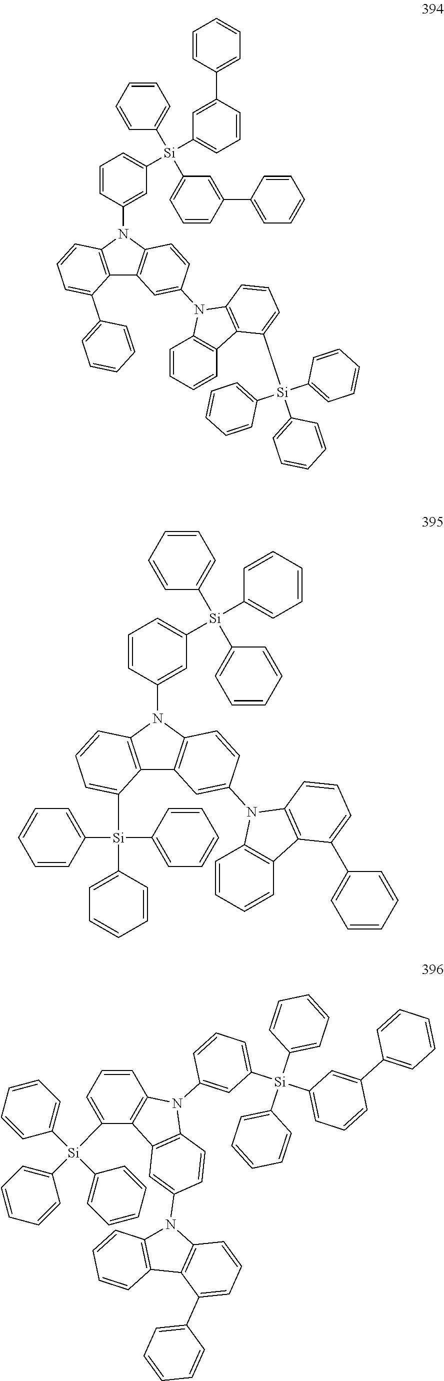 [SMILES: c1ccc(-c2cccc([Si](c3ccccc3)(c3cccc(-c4ccccc4)c3)c3cccc(-n4c5ccc(-n6c7ccccc7c7c([Si](c8ccccc8)(c8ccccc8)c8ccccc8)cccc76)cc5c5c(-c6ccccc6)cccc54)c3)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)cc1 ; c1ccc(-c2cccc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1] 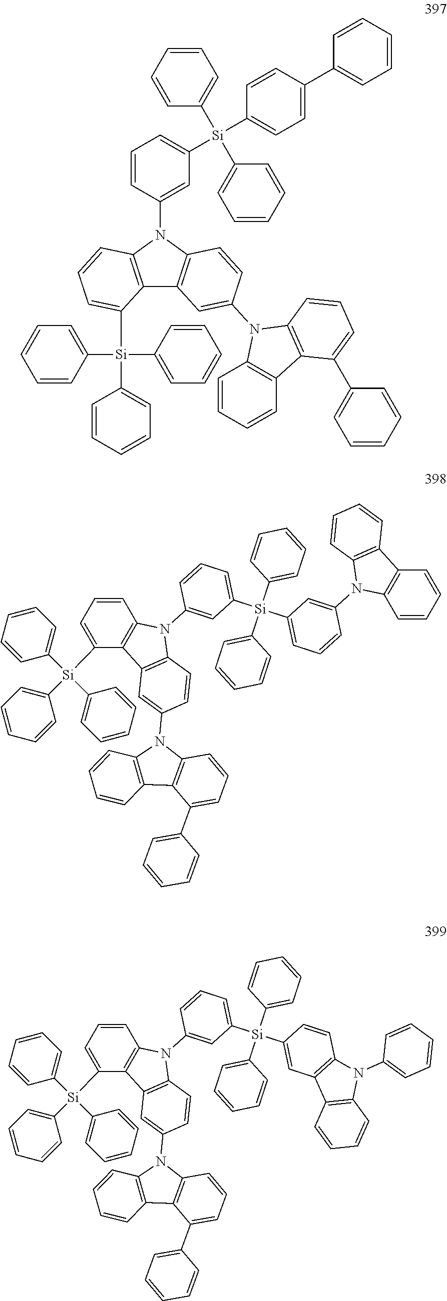 [SMILES: c1ccc(-c2ccc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)cc2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3cccc(-n4c5ccccc5c5ccccc54)c3)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4c(c3)c3ccccc3n4-c3ccccc3)c2)cc1] 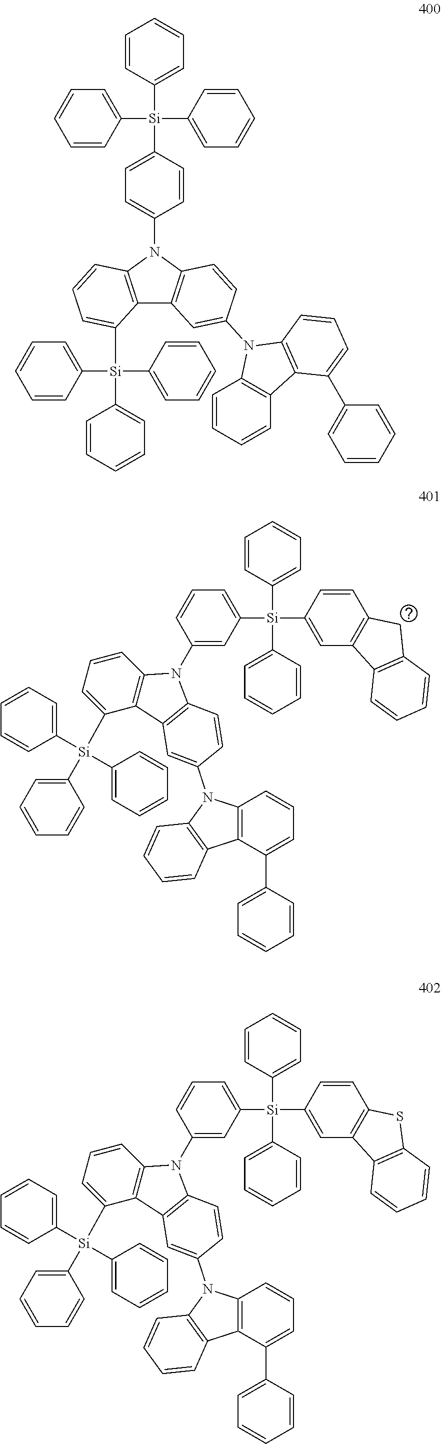 [SMILES: c1ccc(-c2cccc3c2c2ccccc2n3-c2ccc3c(c2)c2c([Si](c4ccccc4)(c4ccccc4)c4ccccc4)cccc2n3-c2ccc([Si](c3ccccc3)(c3ccccc3)c3ccccc3)cc2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4c(c3)-c3ccccc3C4)c2)cc1 ; c1ccc(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4sc5ccccc5c4c3)c2)cc1] 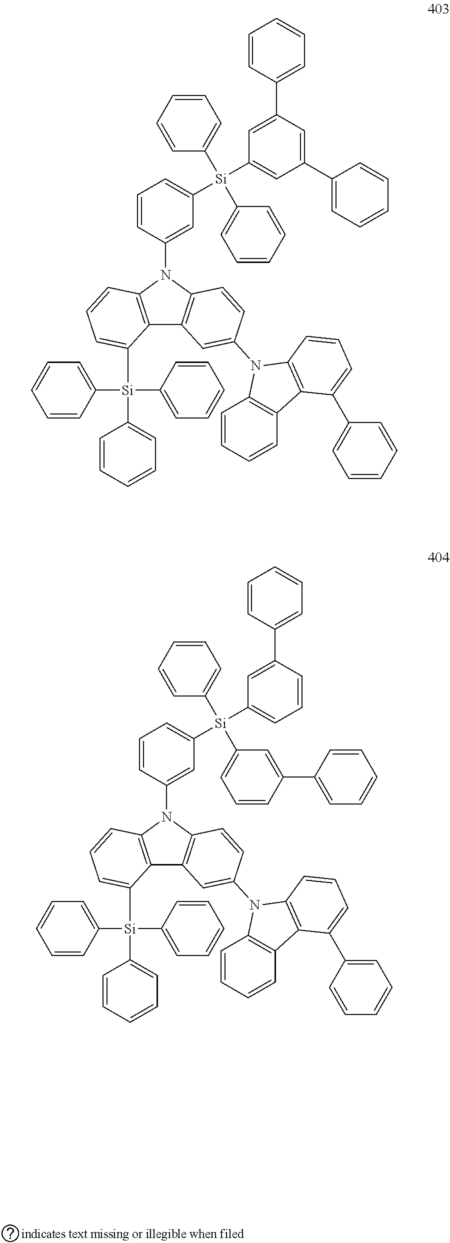 [SMILES: c1ccc(-c2cc(-c3ccccc3)cc([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1 ; c1ccc(-c2cccc([Si](c3ccccc3)(c3cccc(-c4ccccc4)c3)c3cccc(-n4c5ccc(-n6c7ccccc7c7c(-c8ccccc8)cccc76)cc5c5c([Si](c6ccccc6)(c6ccccc6)c6ccccc6)cccc54)c3)c2)cc1] 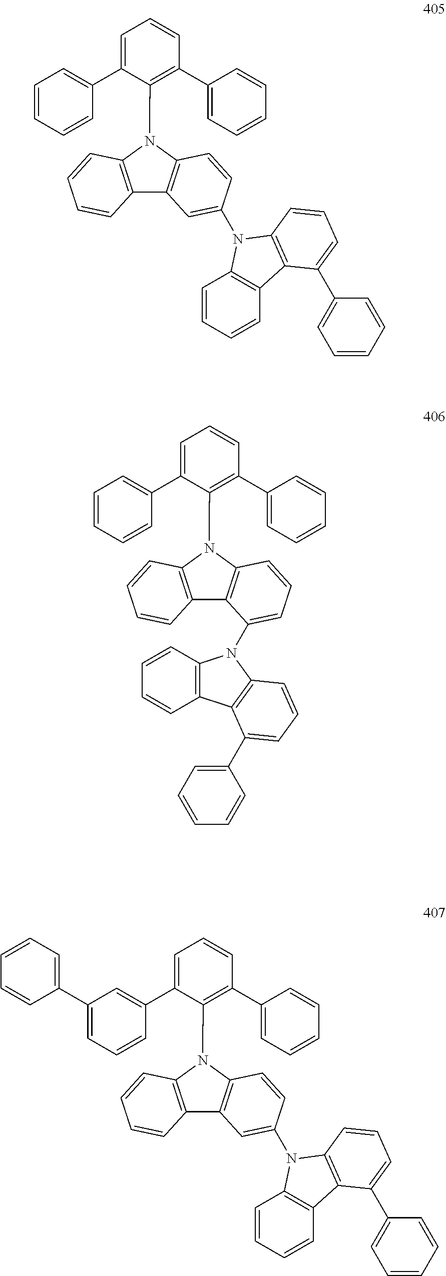 [SMILES: c1ccc(-c2cccc(-c3ccccc3)c2Cn2c3ccccc3c3cc(-n4c5ccccc5c5c(-c6ccccc6)cccc54)ccc32)cc1 ; c1ccc(-c2cccc(-c3ccccc3)c2Cn2c3ccccc3c3c(-n4c5ccccc5c5c(-c6ccccc6)cccc54)cccc32)cc1 ; c1ccc(-c2cccc(-c3cccc(-c4ccccc4)c3Cn3c4ccccc4c4cc(-n5c6ccccc6c6c(-c7ccccc7)cccc65)ccc43)c2)cc1] 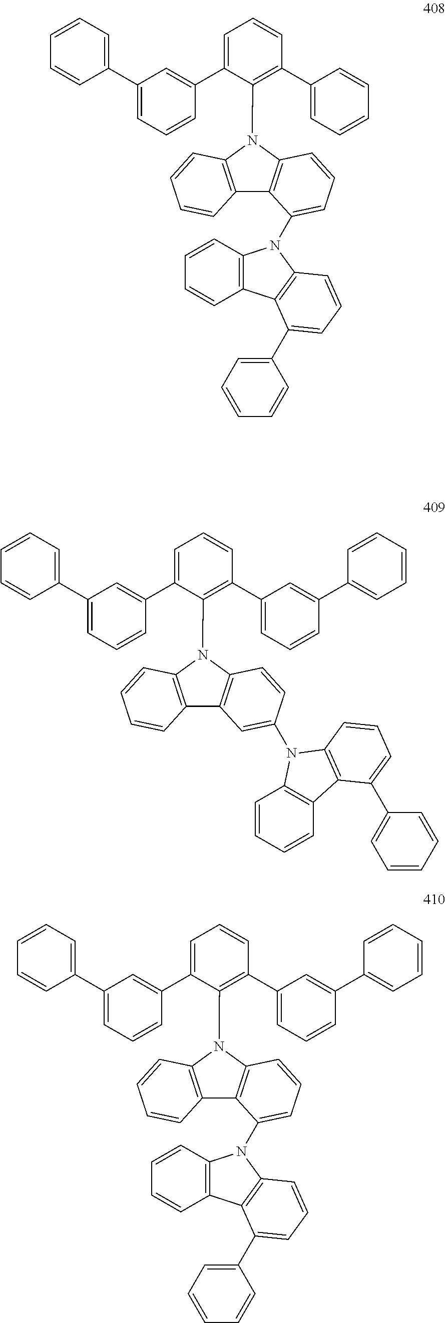 [SMILES: c1ccc(-c2cccc(-c3cccc(-c4ccccc4)c3Cn3c4ccccc4c4c(-n5c6ccccc6c6c(-c7ccccc7)cccc65)cccc43)c2)cc1 ; c1ccc(-c2cccc(-c3cccc(-c4cccc(-c5ccccc5)c4)c3Cn3c4ccccc4c4cc(-n5c6ccccc6c6c(-c7ccccc7)cccc65)ccc43)c2)cc1 ; c1ccc(-c2cccc(-c3cccc(-c4cccc(-c5ccccc5)c4)c3Cn3c4ccccc4c4c(-n5c6ccccc6c6c(-c7ccccc7)cccc65)cccc43)c2)cc1] 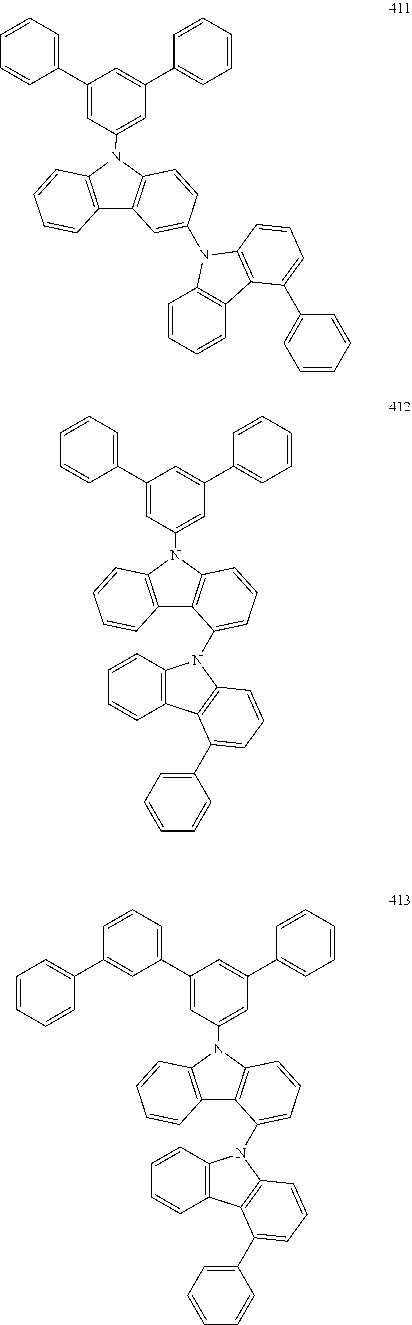 [SMILES: c1ccc(-c2cc(-c3ccccc3)cc(-n3c4ccccc4c4cc(-n5c6ccccc6c6c(-c7ccccc7)cccc65)ccc43)c2)cc1 ; c1ccc(-c2cc(-c3ccccc3)cc(-n3c4ccccc4c4c(-n5c6ccccc6c6c(-c7ccccc7)cccc65)cccc43)c2)cc1 ; c1ccc(-c2cccc(-c3cc(-c4ccccc4)cc(-n4c5ccccc5c5c(-n6c7ccccc7c7c(-c8ccccc8)cccc76)cccc54)c3)c2)cc1] 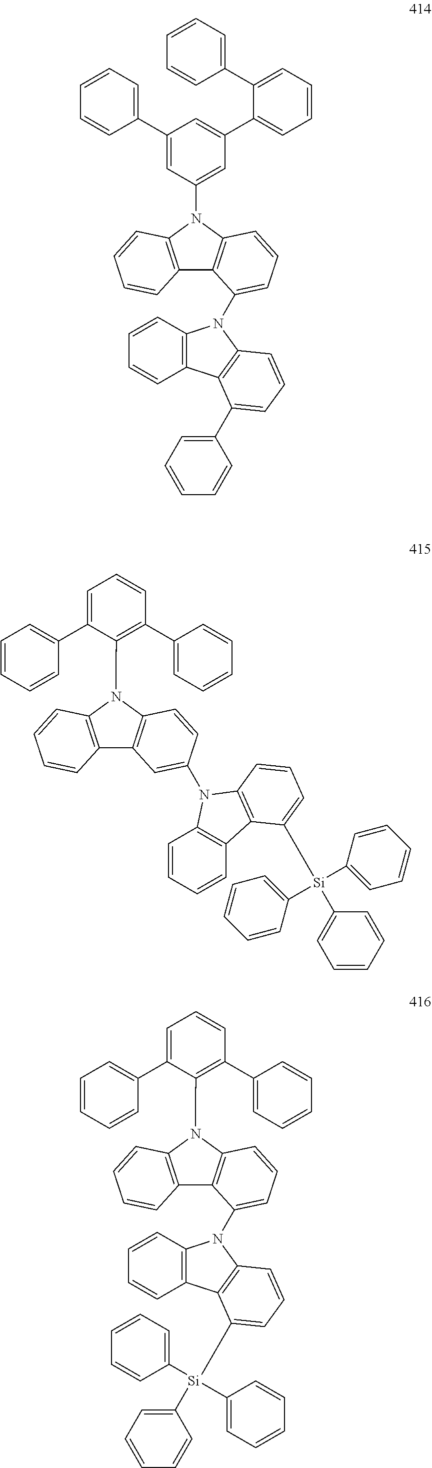 [SMILES: c1ccc(-c2cc(-c3ccccc3-c3ccccc3)cc(-n3c4ccccc4c4c(-n5c6ccccc6c6c(-c7ccccc7)cccc65)cccc43)c2)cc1 ; c1ccc(-c2cccc(-c3ccccc3)c2Cn2c3ccccc3c3cc(-n4c5ccccc5c5c(C[Si](c6ccccc6)(c6ccccc6)c6ccccc6)cccc54)ccc32)cc1 ; c1ccc(-c2cccc(-c3ccccc3)c2Cn2c3ccccc3c3c(-n4c5ccccc5c5c(C[Si](c6ccccc6)(c6ccccc6)c6ccccc6)cccc54)cccc32)cc1] 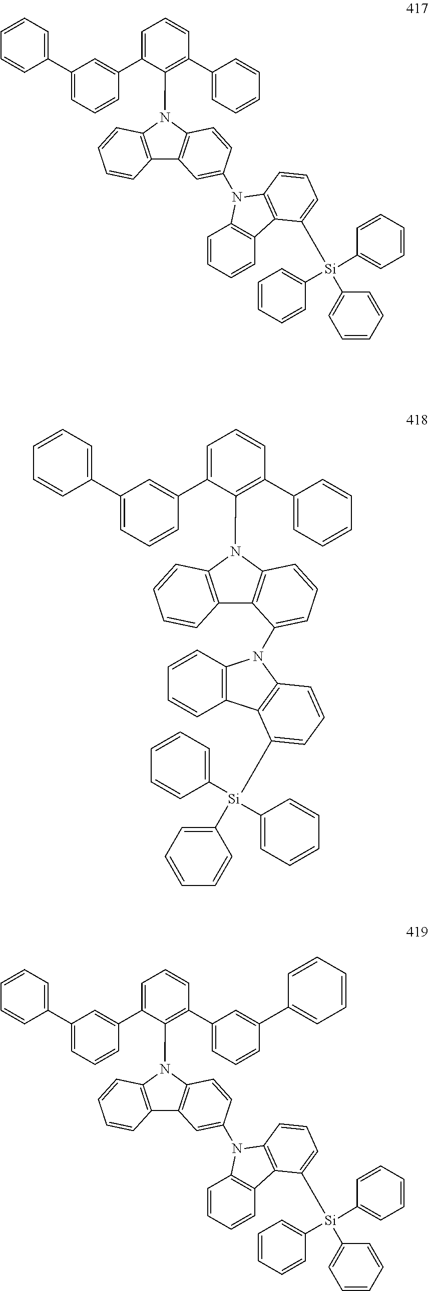 [SMILES: c1ccc(-c2cccc(-c3cccc(-c4ccccc4)c3Cn3c4ccccc4c4cc(-n5c6ccccc6c6c(C[Si](c7ccccc7)(c7ccccc7)c7ccccc7)cccc65)ccc43)c2)cc1 ; c1ccc(-c2cccc(-c3cccc(-c4ccccc4)c3Cn3c4ccccc4c4c(-n5c6ccccc6c6c(C[Si](c7ccccc7)(c7ccccc7)c7ccccc7)cccc65)cccc43)c2)cc1 ; c1ccc(-c2cccc(-c3cccc(-c4cccc(-c5ccccc5)c4)c3Cn3c4ccccc4c4cc(-n5c6ccccc6c6c(C[Si](c7ccccc7)(c7ccccc7)c7ccccc7)cccc65)ccc43)c2)cc1] 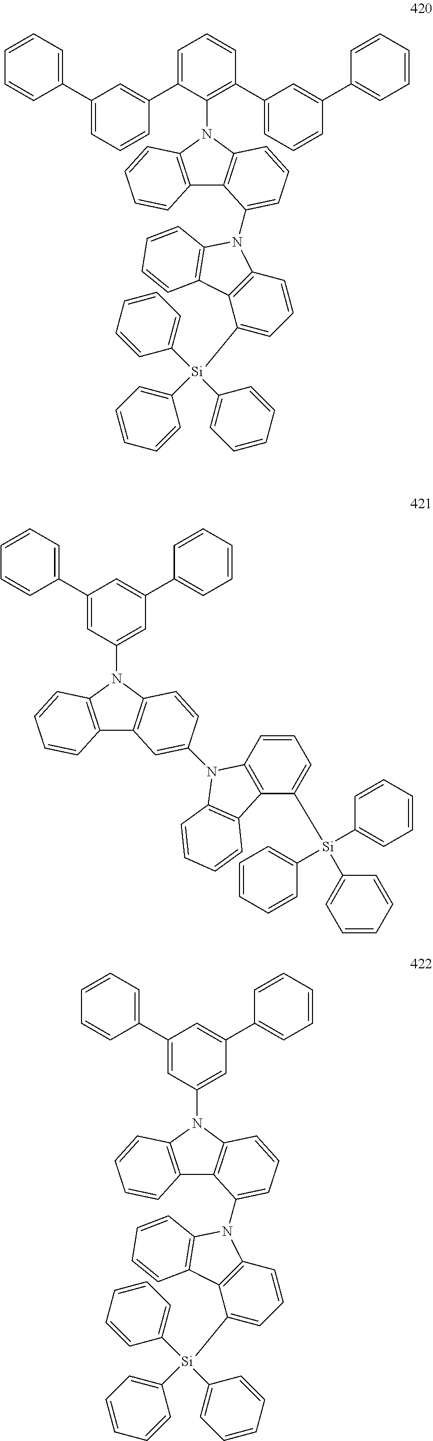 [SMILES: c1ccc(-c2cccc(-c3cccc(-c4cccc(-c5ccccc5)c4)c3-n3c4ccccc4c4c(-n5c6ccccc6c6c([Si](c7ccccc7)(c7ccccc7)c7ccccc7)cccc65)cccc43)c2)cc1 ; c1ccc(-c2cc(-c3ccccc3)cc(-n3c4ccccc4c4cc(-n5c6ccccc6c6c([Si](c7ccccc7)(c7ccccc7)c7ccccc7)cccc65)ccc43)c2)cc1 ; c1ccc(-c2cc(-c3ccccc3)cc(-n3c4ccccc4c4c(-n5c6ccccc6c6c([Si](c7ccccc7)(c7ccccc7)c7ccccc7)cccc65)cccc43)c2)cc1] 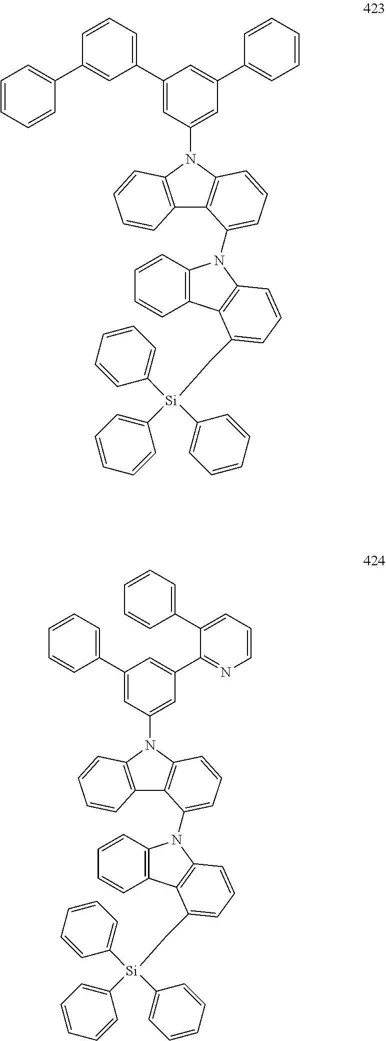 [SMILES: c1ccc(-c2cccc(-c3cc(-c4ccccc4)cc(-n4c5ccccc5c5c(-n6c7ccccc7c7c(C[Si](c8ccccc8)(c8ccccc8)c8ccccc8)cccc76)cccc54)c3)c2)cc1 ; c1ccc(-c2cc(-c3ncccc3-c3ccccc3)cc(-n3c4ccccc4c4c(-n5c6ccccc6c6c(C[Si](c7ccccc7)(c7ccccc7)c7ccccc7)cccc65)cccc43)c2)cc1] 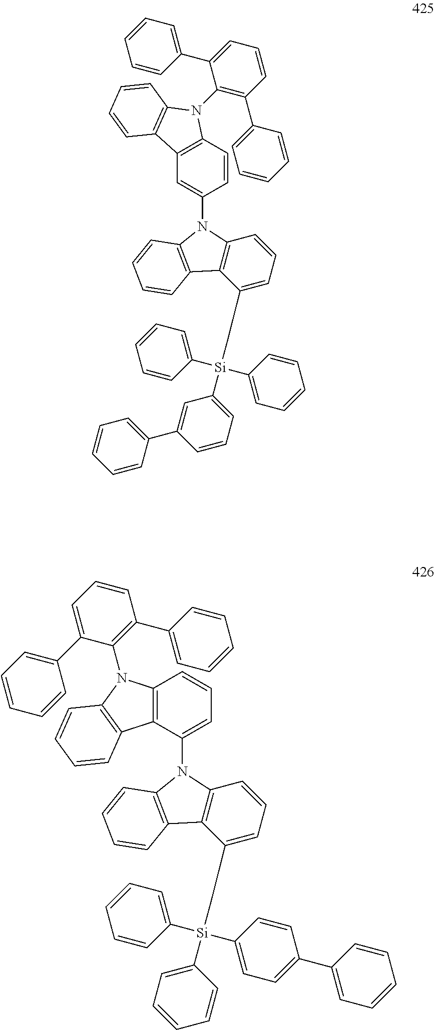 [SMILES: c1ccc(-c2cccc([Si](Cc3cccc4c3c3ccccc3n4-c3ccc4c(c3)c3ccccc3n4-c3c(-c4ccccc4)cccc3-c3ccccc3)(c3ccccc3)c3ccccc3)c2)cc1 ; c1ccc(-c2ccc([Si](Cc3cccc4c3c3ccccc3n4-c3cccc4c3c3ccccc3n4-c3c(-c4ccccc4)cccc3-c3ccccc3)(c3ccccc3)c3ccccc3)cc2)cc1] 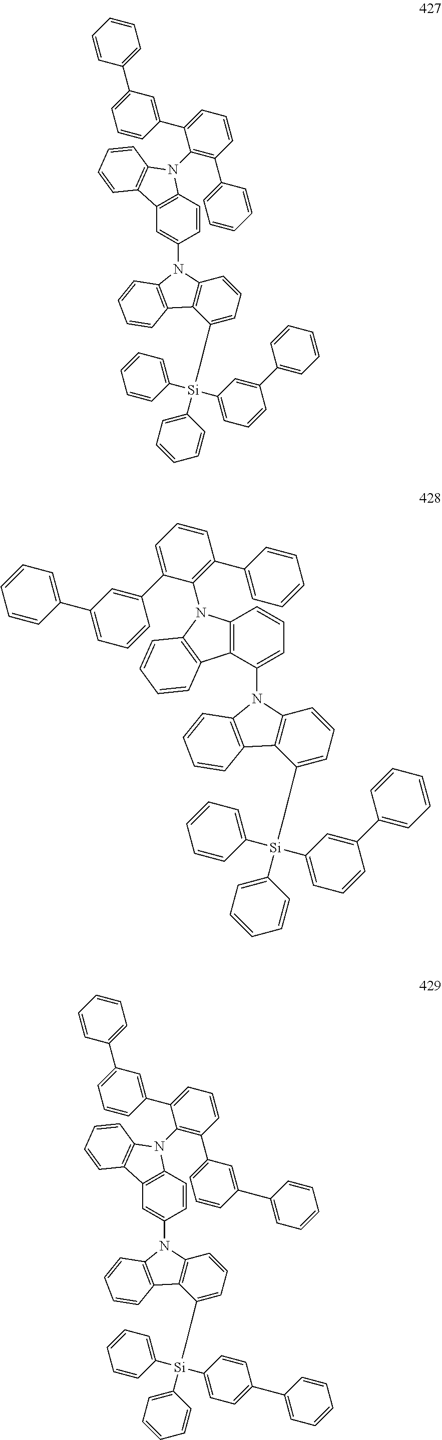 [SMILES: c1ccc(-c2cccc(-c3cccc(-c4ccccc4)c3-n3c4ccccc4c4cc(-n5c6ccccc6c6c(C[Si](c7ccccc7)(c7ccccc7)c7cccc(-c8ccccc8)c7)cccc65)ccc43)c2)cc1 ; c1ccc(-c2cccc(-c3cccc(-c4ccccc4)c3-n3c4ccccc4c4c(-n5c6ccccc6c6c(C[Si](c7ccccc7)(c7ccccc7)c7cccc(-c8ccccc8)c7)cccc65)cccc43)c2)cc1 ; c1ccc(-c2ccc([Si](Cc3cccc4c3c3ccccc3n4-c3ccc4c(c3)c3ccccc3n4-c3c(-c4cccc(-c5ccccc5)c4)cccc3-c3cccc(-c4ccccc4)c3)(c3ccccc3)c3ccccc3)cc2)cc1] 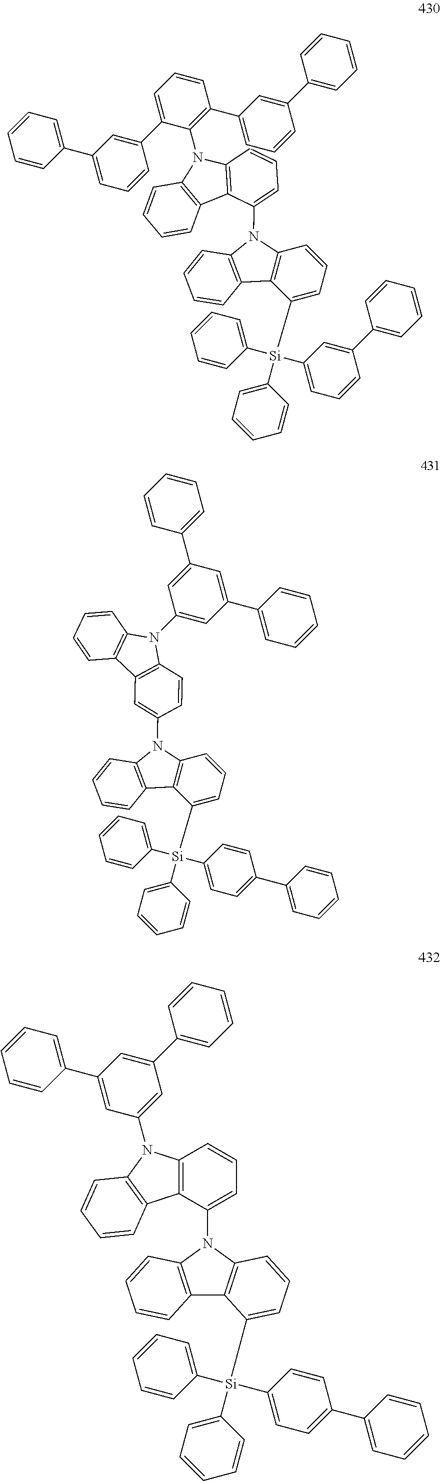 [SMILES: c1ccc(-c2cccc(-c3cccc(-c4cccc(-c5ccccc5)c4)c3-n3c4ccccc4c4c(-n5c6ccccc6c6c([Si](c7ccccc7)(c7ccccc7)c7cccc(-c8ccccc8)c7)cccc65)cccc43)c2)cc1 ; c1ccc(-c2ccc([Si](c3ccccc3)(c3ccccc3)c3cccc4c3c3ccccc3n4-c3ccc4c(c3)c3ccccc3n4-c3cc(-c4ccccc4)cc(-c4ccccc4)c3)cc2)cc1 ; c1ccc(-c2ccc([Si](Cc3cccc4c3c3ccccc3n4-c3cccc4c3c3ccccc3n4-c3cc(-c4ccccc4)cc(-c4ccccc4)c3)(c3ccccc3)c3ccccc3)cc2)cc1] 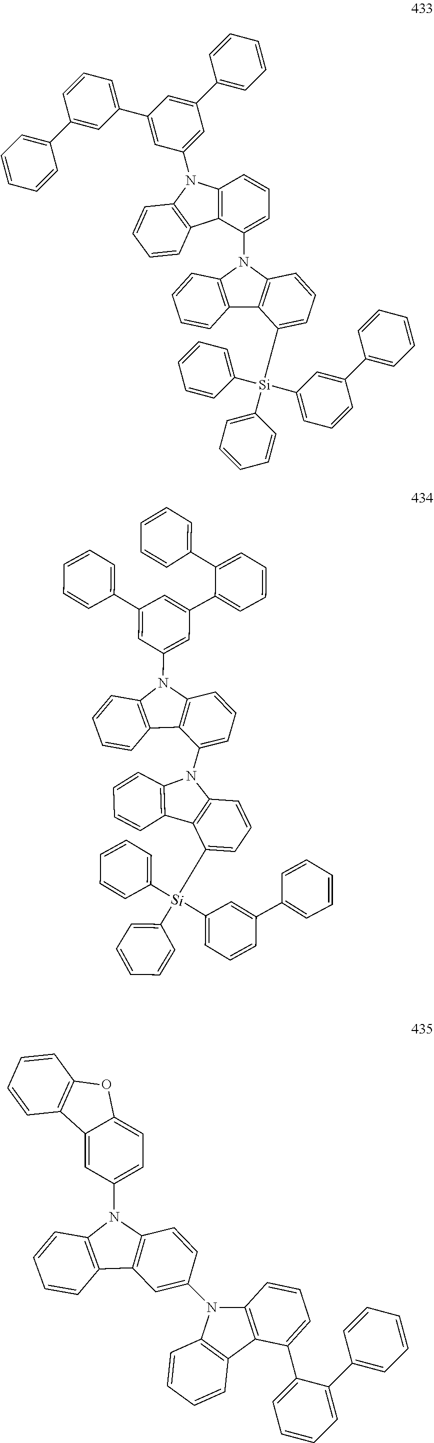 [SMILES: c1ccc(-c2cccc(-c3cc(-c4ccccc4)cc(-n4c5ccccc5c5c(-n6c7ccccc7c7c(C[Si](c8ccccc8)(c8ccccc8)c8cccc(-c9ccccc9)c8)cccc76)cccc54)c3)c2)cc1 ; c1ccc(-c2cc(-c3ccccc3-c3ccccc3)cc(-n3c4ccccc4c4c(-n5c6ccccc6c6c(C[Si](c7ccccc7)(c7ccccc7)c7cccc(-c8ccccc8)c7)cccc65)cccc43)c2)cc1 ; c1ccc(-c2ccccc2-c2cccc3c2c2ccccc2n3-c2ccc3c(c2)c2ccccc2n3-c2ccc3oc4ccccc4c3c2)cc1] 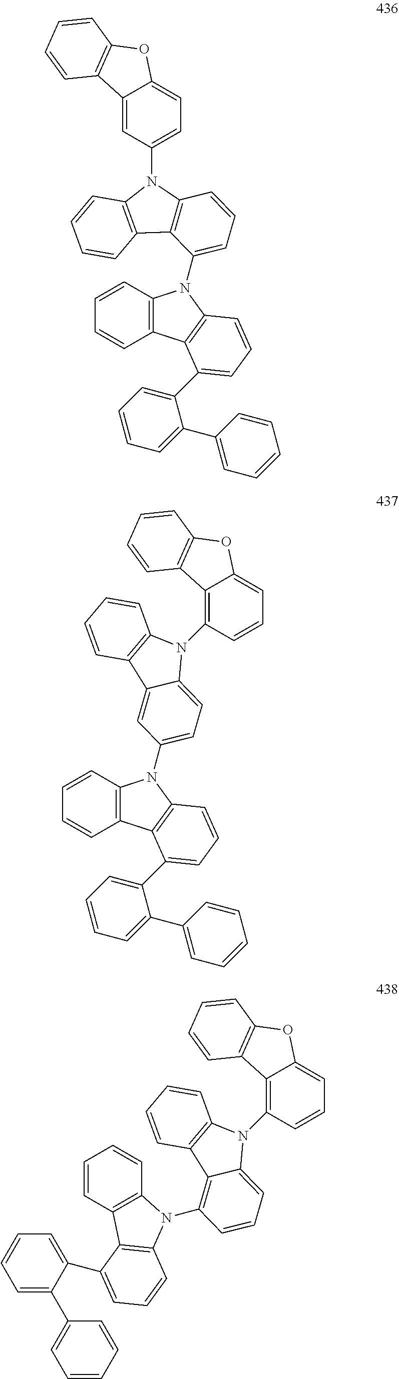 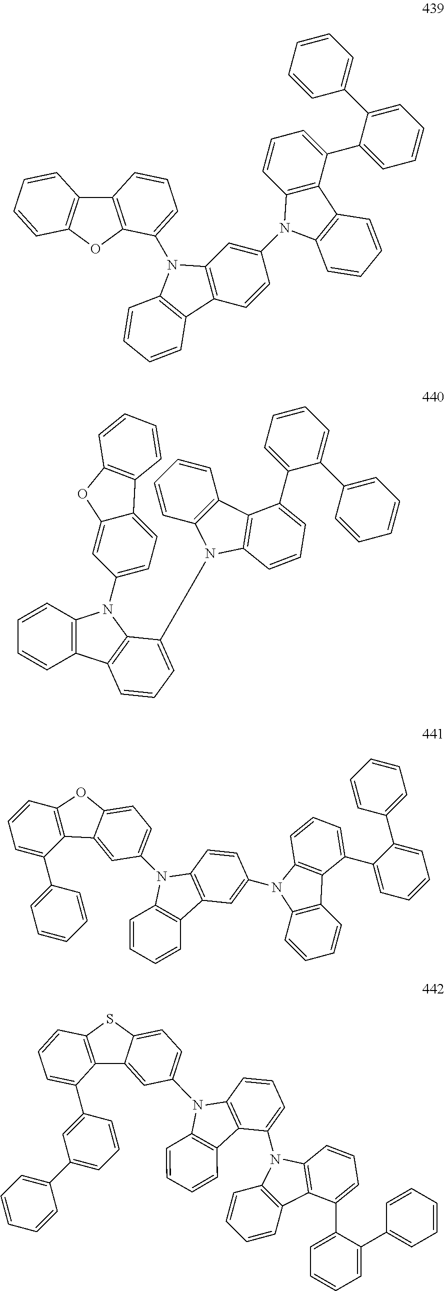 [SMILES: c1ccc(-c2ccccc2-c2cccc3c2c2ccccc2n3-c2ccc3c4ccccc4n(-c4cccc5c4oc4ccccc45)c3c2)cc1 ; c1ccc(-c2ccccc2-c2cccc3c2c2ccccc2n3CCc2cccc3c4ccccc4n(-c4ccc5c(c4)oc4ccccc45)c23)cc1 ; c1ccc(-c2ccccc2-c2cccc3c2c2ccccc2n3-c2ccc3c(c2)c2ccccc2n3-c2ccc3oc4cccc(-c5ccccc5)c4c3c2)cc1 ; c1ccc(-c2cccc(-c3cccc4sc5ccc(-n6c7ccccc7c7c(-n8c9ccccc9c9c(-c%10ccccc%10-c%10ccccc%10)cccc98)cccc76)cc5c34)c2)cc1] 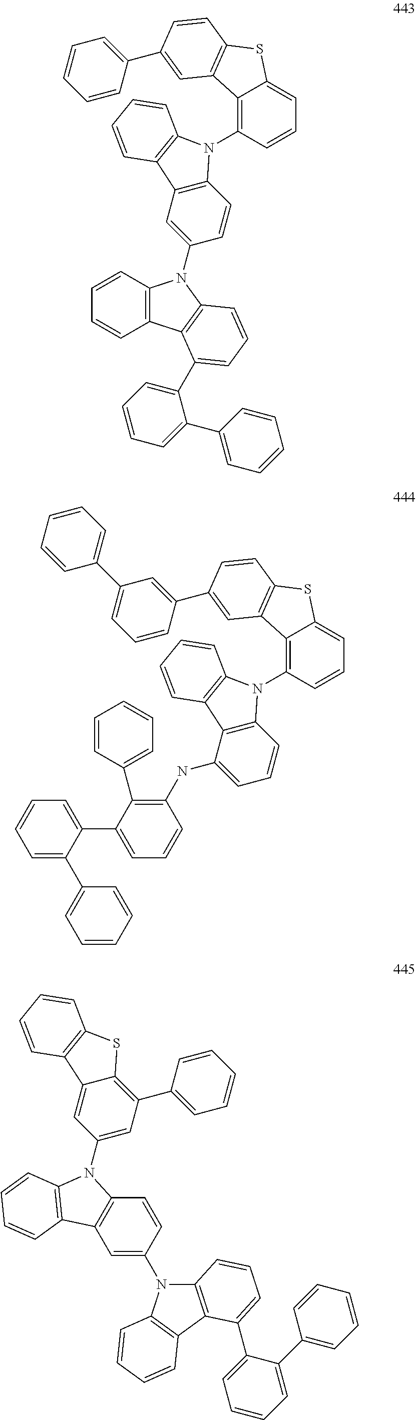 [SMILES: c1ccc(-c2ccc3sc4cccc(-n5c6ccccc6c6cc(-n7c8ccccc8c8c(-c9ccccc9-c9ccccc9)cccc87)ccc65)c4c3c2)cc1 ; c1ccc(-c2cccc(-c3ccc4sc5cccc(-n6c7ccccc7c7c(Nc8cccc(-c9ccccc9-c9ccccc9)c8-c8ccccc8)cccc76)c5c4c3)c2)cc1 ; c1ccc(-c2ccccc2-c2cccc3c2c2ccccc2n3-c2ccc3c(c2)c2ccccc2n3-c2cc(-c3ccccc3)c3sc4ccccc4c3c2)cc1] 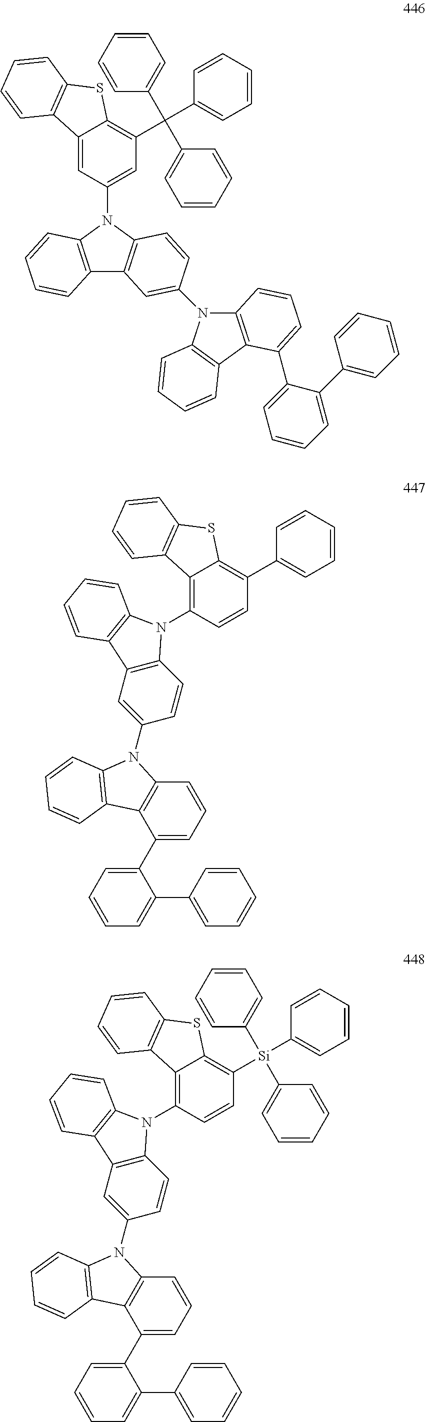 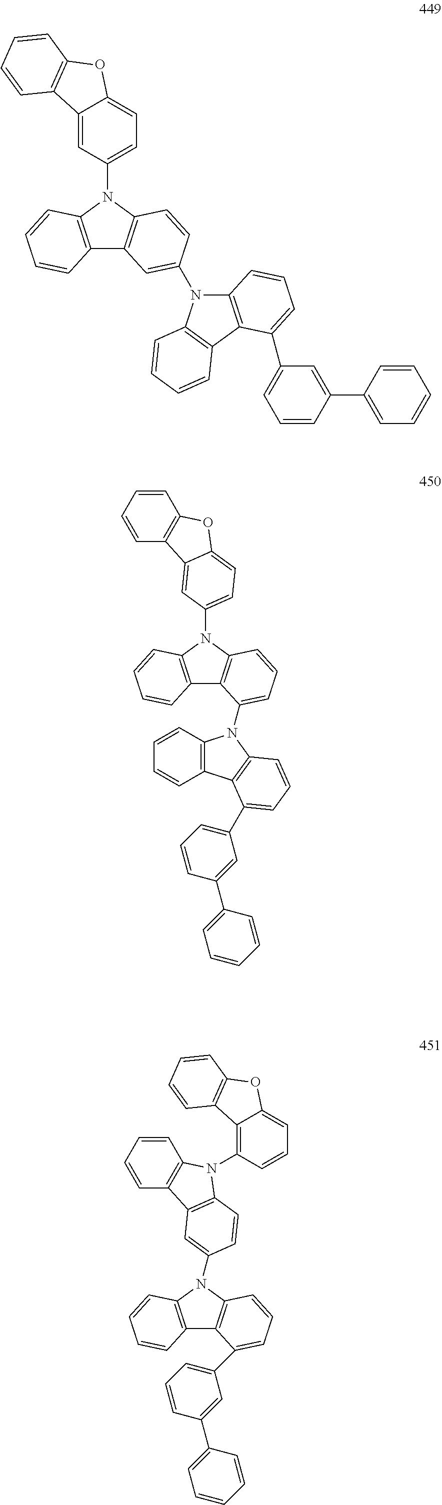 [SMILES: c1ccc(-c2cccc(-c3cccc4c3c3ccccc3n4-c3ccc4c(c3)c3ccccc3n4-c3ccc4oc5ccccc5c4c3)c2)cc1 ; c1ccc(-c2cccc(-c3cccc4c3c3ccccc3n4-c3cccc4c3c3ccccc3n4-c3ccc4oc5ccccc5c4c3)c2)cc1 ; c1ccc(-c2cccc(-c3cccc4c3c3ccccc3n4-c3ccc4c(c3)c3ccccc3n4-c3cccc4oc5ccccc5c34)c2)cc1] 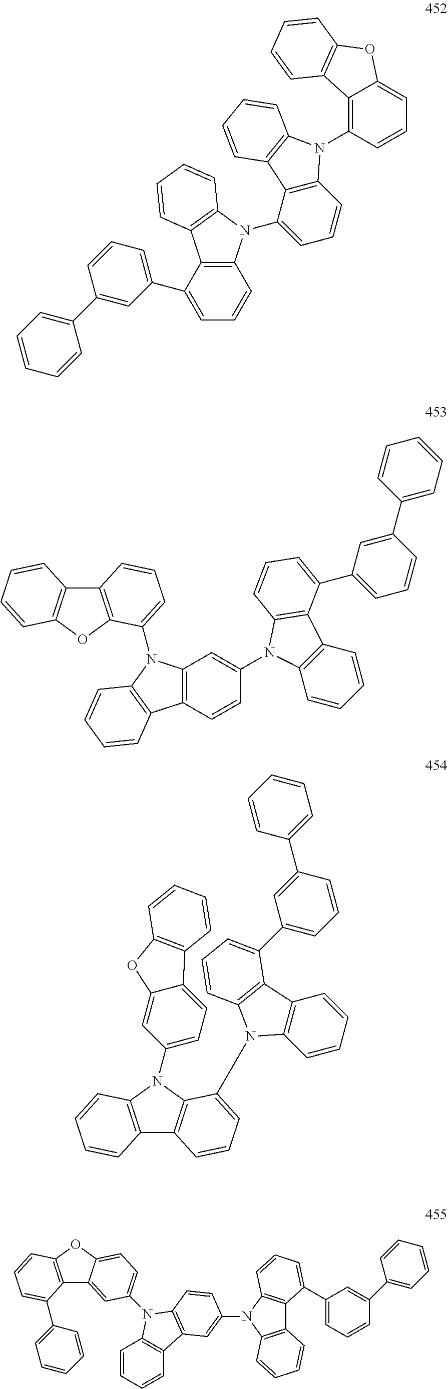 [SMILES: c1ccc(-c2cccc(-c3cccc4c3c3ccccc3n4-c3cccc4c3c3ccccc3n4-c3cccc4oc5ccccc5c34)c2)cc1 ; c1ccc(-c2cccc(-c3cccc4c3c3ccccc3n4-c3ccc4c5ccccc5n(-c5cccc6c5oc5ccccc56)c4c3)c2)cc1 ; c1ccc(-c2cccc(-c3cccc4c3c3ccccc3n4Cc3cccc4c5ccccc5n(-c5ccc6c(c5)oc5ccccc56)c34)c2)cc1 ; c1ccc(-c2cccc(-c3cccc4c3c3ccccc3n4-c3ccc4c(c3)c3ccccc3n4-c3ccc4oc5cccc(-c6ccccc6)c5c4c3)c2)cc1] 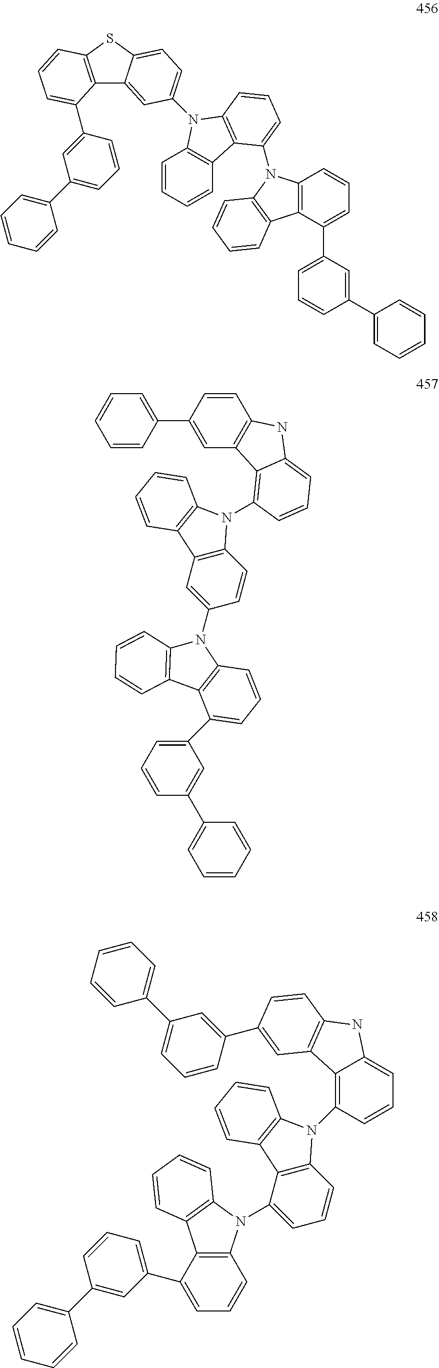 [SMILES: c1ccc(-c2cccc(-c3cccc4sc5ccc(-n6c7ccccc7c7c(-n8c9ccccc9c9c(-c%10cccc(-c%11ccccc%11)c%10)cccc98)cccc76)cc5c34)c2)cc1 ; c1ccc(-c2cccc(-c3cccc4c3c3ccccc3n4-c3ccc4c(c3)c3ccccc3n4-c3cccc4[nH]c5ccc(-c6ccccc6)cc5c34)c2)cc1 ; c1ccc(-c2cccc(-c3ccc4[nH]c5cccc(-n6c7ccccc7c7c(-n8c9ccccc9c9c(-c%10cccc(-c%11ccccc%11)c%10)cccc98)cccc76)c5c4c3)c2)cc1] 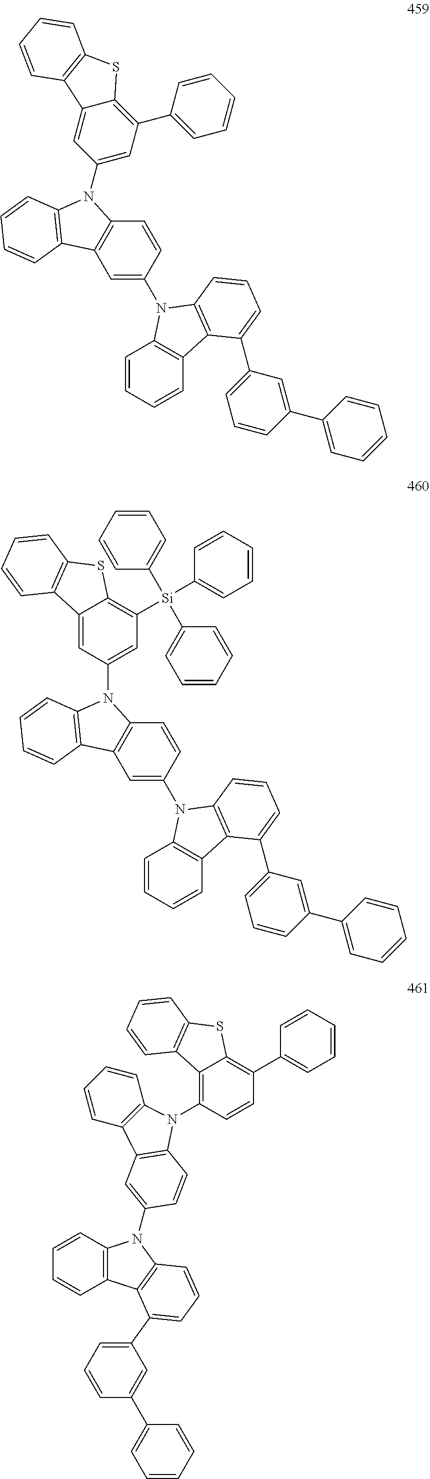 [SMILES: c1ccc(-c2cccc(-c3cccc4c3c3ccccc3n4-c3ccc4c(c3)c3ccccc3n4-c3cc(-c4ccccc4)c4sc5ccccc5c4c3)c2)cc1 ; c1ccc(-c2cccc(-c3cccc4c3c3ccccc3n4-c3ccc4c(c3)c3ccccc3n4-c3cc([Si](c4ccccc4)(c4ccccc4)c4ccccc4)c4sc5ccccc5c4c3)c2)cc1 ; c1ccc(-c2cccc(-c3cccc4c3c3ccccc3n4-c3ccc4c(c3)c3ccccc3n4-c3ccc(-c4ccccc4)c4sc5ccccc5c34)c2)cc1] 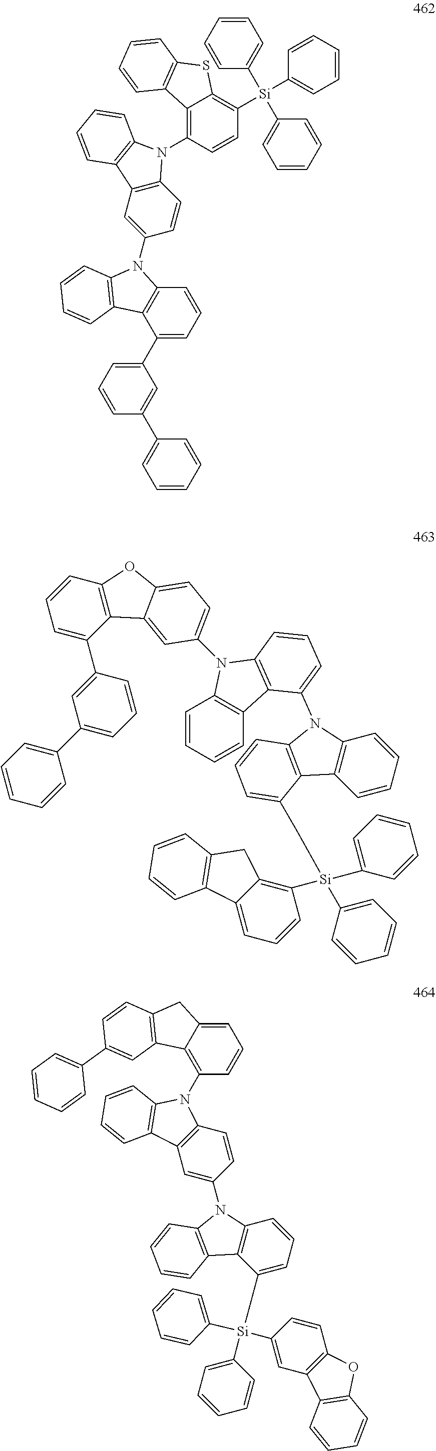 [SMILES: c1ccc(-c2cccc(-c3cccc4c3c3ccccc3n4-c3ccc4c(c3)c3ccccc3n4-c3ccc([Si](c4ccccc4)(c4ccccc4)c4ccccc4)c4sc5ccccc5c34)c2)cc1 ; c1ccc(-c2cccc(-c3cccc4oc5ccc(-n6c7ccccc7c7c(-n8c9ccccc9c9c(C[Si](c%10ccccc%10)(c%10ccccc%10)c%10cccc%11c%10Cc%10ccccc%10-%11)cccc98)cccc76)cc5c34)c2)cc1 ; c1ccc(-c2ccc3c(c2)-c2c(cccc2-n2c4ccccc4c4cc(-n5c6ccccc6c6c(C[Si](c7ccccc7)(c7ccccc7)c7ccc8oc9ccccc9c8c7)cccc65)ccc42)C3)cc1] 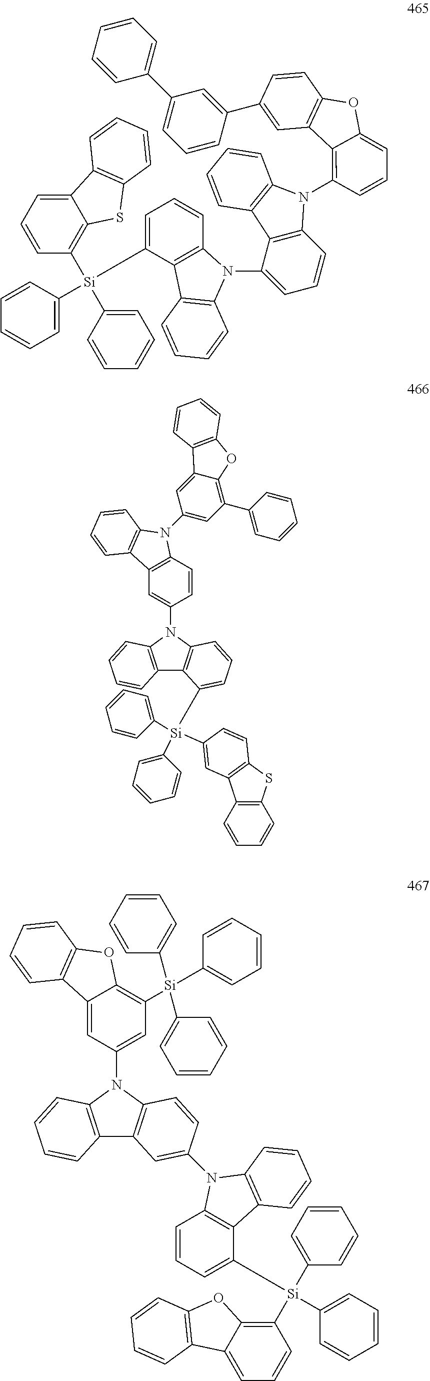 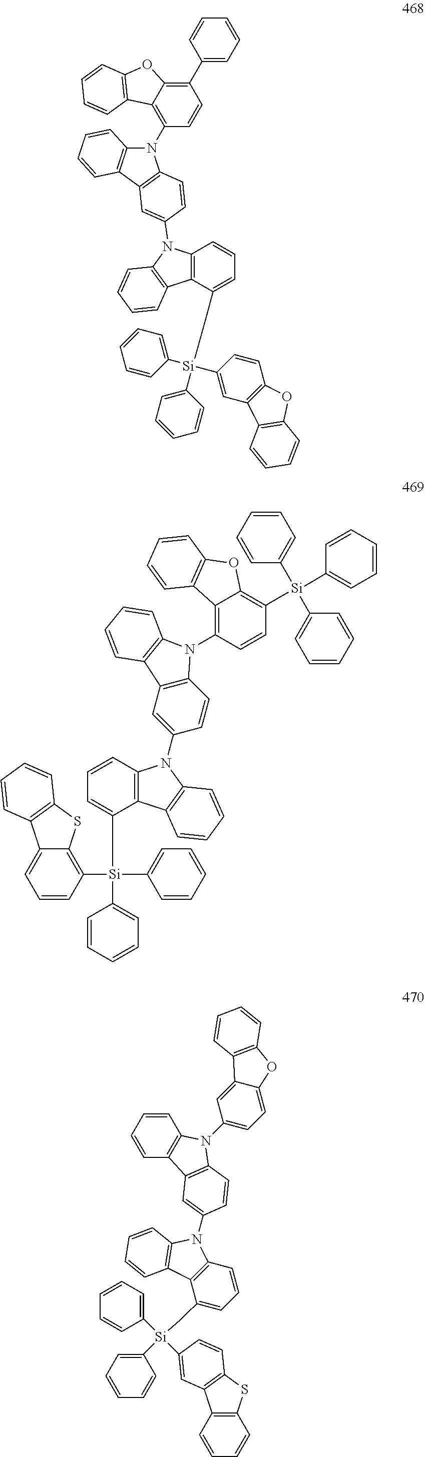 [SMILES: c1ccc(-c2ccc(-n3c4ccccc4c4cc(-n5c6ccccc6c6c(C[Si](c7ccccc7)(c7ccccc7)c7ccc8oc9ccccc9c8c7)cccc65)ccc43)c3c2oc2ccccc23)cc1 ; c1ccc([Si](Cc2ccc(-n3c4ccccc4c4cc(-n5c6ccccc6c6c(C[Si](c7ccccc7)(c7ccccc7)c7cccc8c7sc7ccccc78)cccc65)ccc43)c3c2oc2ccccc23)(c2ccccc2)c2ccccc2)cc1 ; c1ccc([Si](Cc2cccc3c2c2ccccc2n3-c2ccc3c(c2)c2ccccc2n3-c2ccc3oc4ccccc4c3c2)(c2ccccc2)c2ccc3sc4ccccc4c3c2)cc1] 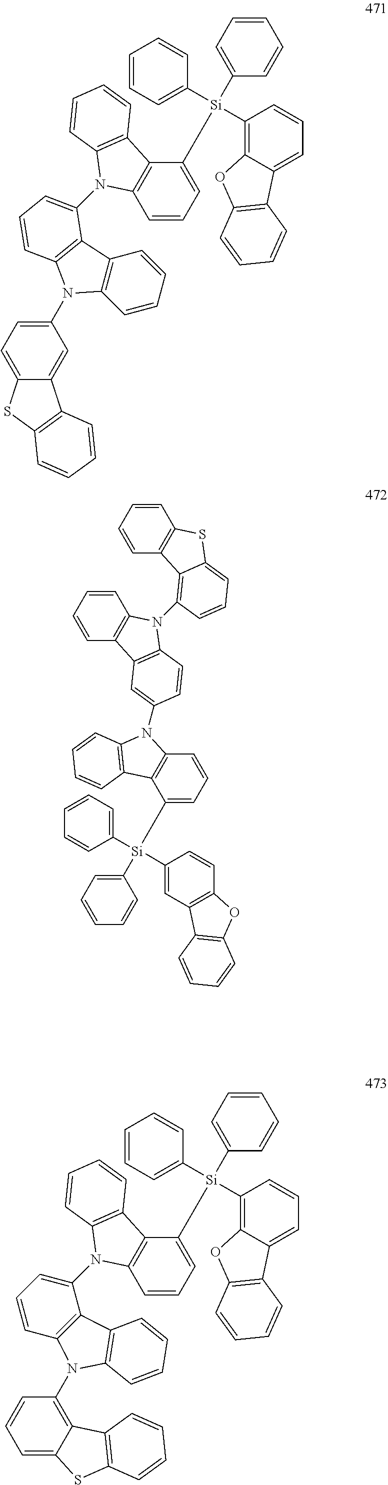 [SMILES: c1ccc([Si](Cc2cccc3c2c2ccccc2n3-c2cccc3c2c2ccccc2n3-c2ccc3sc4ccccc4c3c2)(c2ccccc2)c2cccc3c2oc2ccccc23)cc1 ; c1ccc([Si](Cc2cccc3c2c2ccccc2n3-c2ccc3c(c2)c2ccccc2n3-c2cccc3sc4ccccc4c23)(c2ccccc2)c2ccc3oc4ccccc4c3c2)cc1 ; c1ccc([Si](Cc2cccc3c2c2ccccc2n3-c2cccc3c2c2ccccc2n3-c2cccc3sc4ccccc4c23)(c2ccccc2)c2cccc3c2oc2ccccc23)cc1] 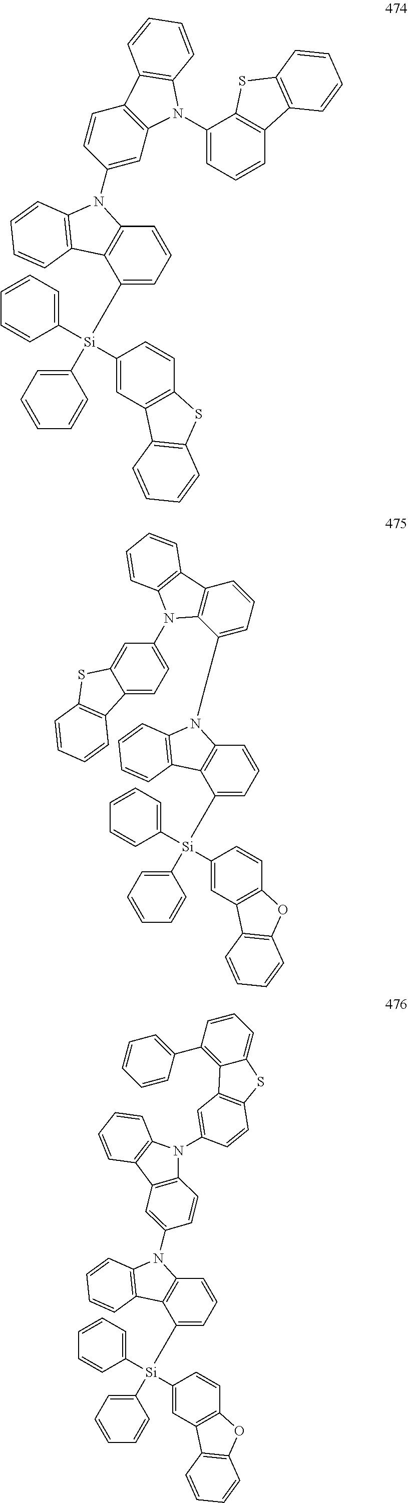 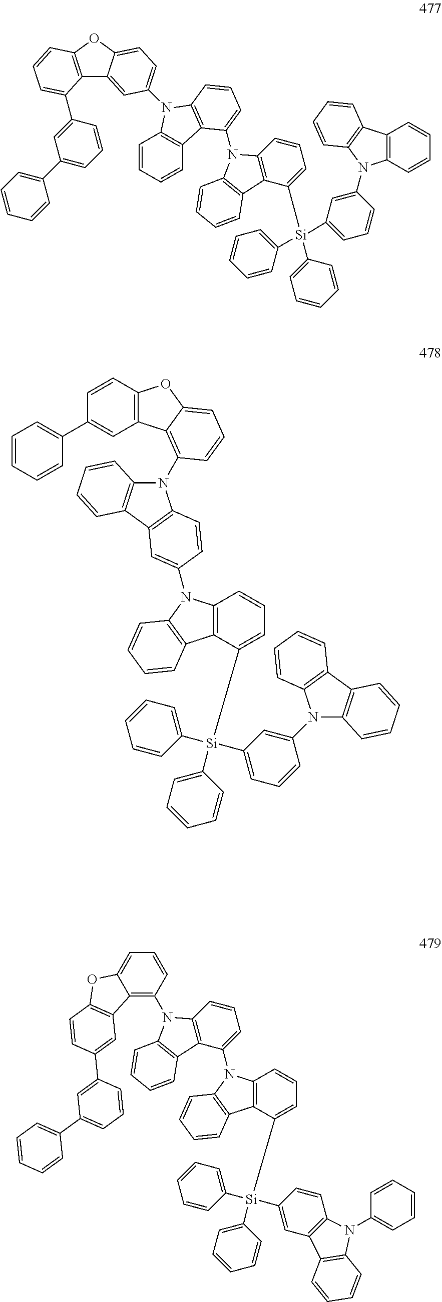 [SMILES: c1ccc(-c2cccc(-c3cccc4oc5ccc(-n6c7ccccc7c7c(-n8c9ccccc9c9c(C[Si](c%10ccccc%10)(c%10ccccc%10)c%10cccc(-n%11c%12ccccc%12c%12ccccc%12%11)c%10)cccc98)cccc76)cc5c34)c2)cc1 ; c1ccc(-c2ccc3oc4cccc(-n5c6ccccc6c6cc(-n7c8ccccc8c8c([Si](c9ccccc9)(c9ccccc9)c9cccc(-n%10c%11ccccc%11c%11ccccc%11%10)c9)cccc87)ccc65)c4c3c2)cc1 ; c1ccc(-c2cccc(-c3ccc4oc5cccc(-n6c7ccccc7c7c(-n8c9ccccc9c9c([Si](c%10ccccc%10)(c%10ccccc%10)c%10ccc%11c(c%10)c%10ccccc%10n%11-c%10ccccc%10)cccc98)cccc76)c5c4c3)c2)cc1] 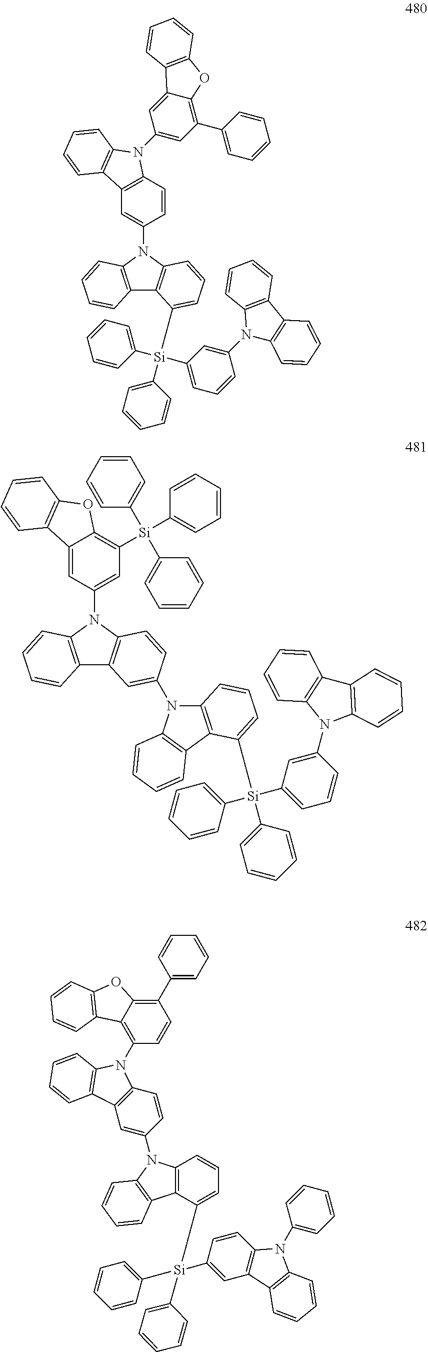 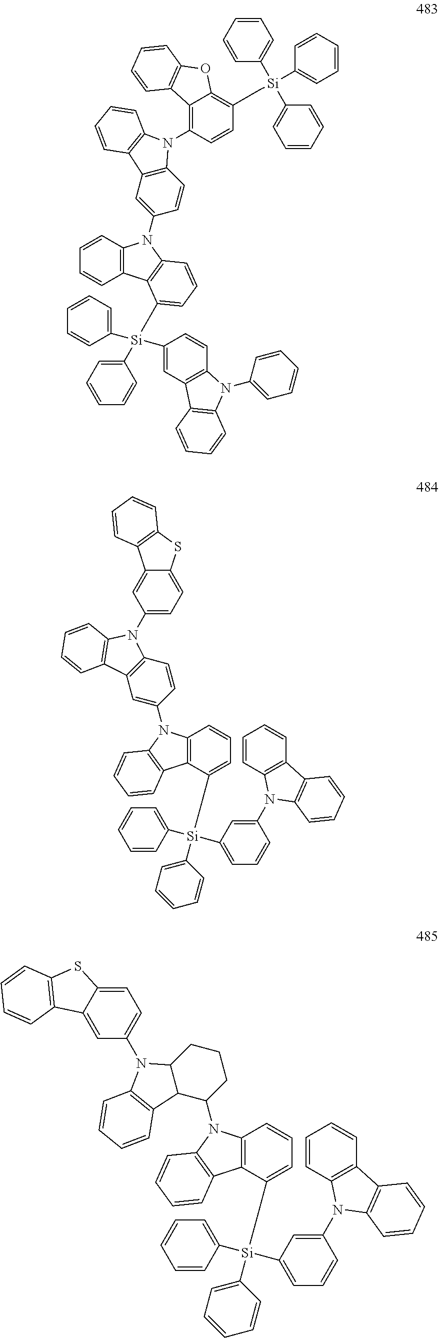 [SMILES: c1ccc(-n2c3ccccc3c3cc([Si](c4ccccc4)(c4ccccc4)c4cccc5c4c4ccccc4n5-c4ccc5c(c4)c4ccccc4n5-c4ccc(C[Si](c5ccccc5)(c5ccccc5)c5ccccc5)c5oc6ccccc6c45)ccc32)cc1 ; c1ccc([Si](Cc2cccc3c2c2ccccc2n3-c2ccc3c(c2)c2ccccc2n3-c2ccc3sc4ccccc4c3c2)(c2ccccc2)c2cccc(-n3c4ccccc4c4ccccc43)c2)cc1 ; c1ccc([Si](Cc2cccc3c2c2ccccc2n3C2CCCC3C2c2ccccc2N3c2ccc3sc4ccccc4c3c2)(c2ccccc2)c2cccc(-n3c4ccccc4c4ccccc43)c2)cc1] 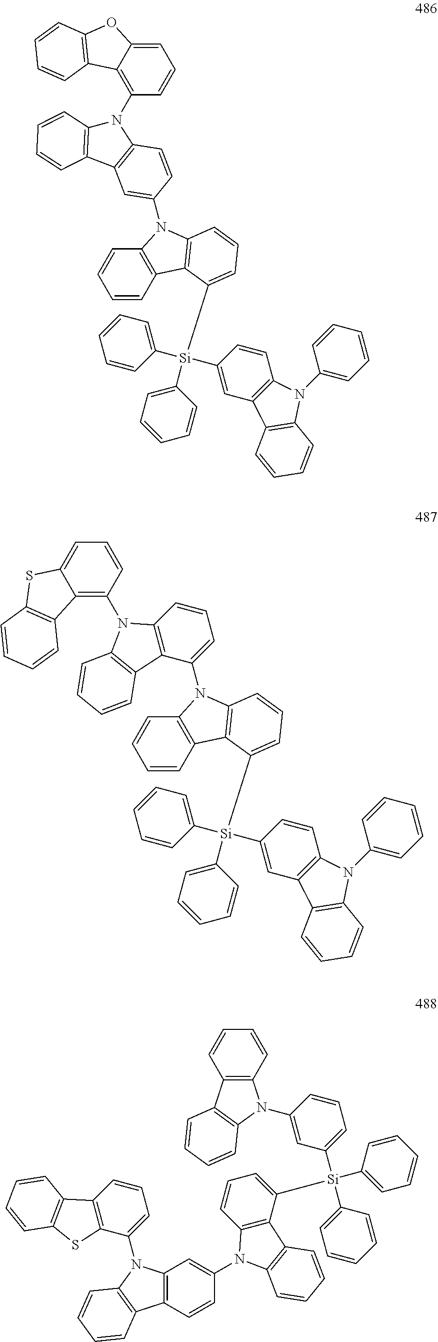 [SMILES: c1ccc(-n2c3ccccc3c3cc([Si](c4ccccc4)(c4ccccc4)c4cccc5c4c4ccccc4n5-c4ccc5c(c4)c4ccccc4n5-c4cccc5oc6ccccc6c45)ccc32)cc1 ; c1ccc(-n2c3ccccc3c3cc([Si](c4ccccc4)(c4ccccc4)c4cccc5c4c4ccccc4n5-c4cccc5c4c4ccccc4n5-c4cccc5sc6ccccc6c45)ccc32)cc1 ; c1ccc([Si](Cc2cccc3c2c2ccccc2n3-c2ccc3c4ccccc4n(-c4cccc5c4sc4ccccc45)c3c2)(c2ccccc2)c2cccc(-n3c4ccccc4c4ccccc43)c2)cc1] 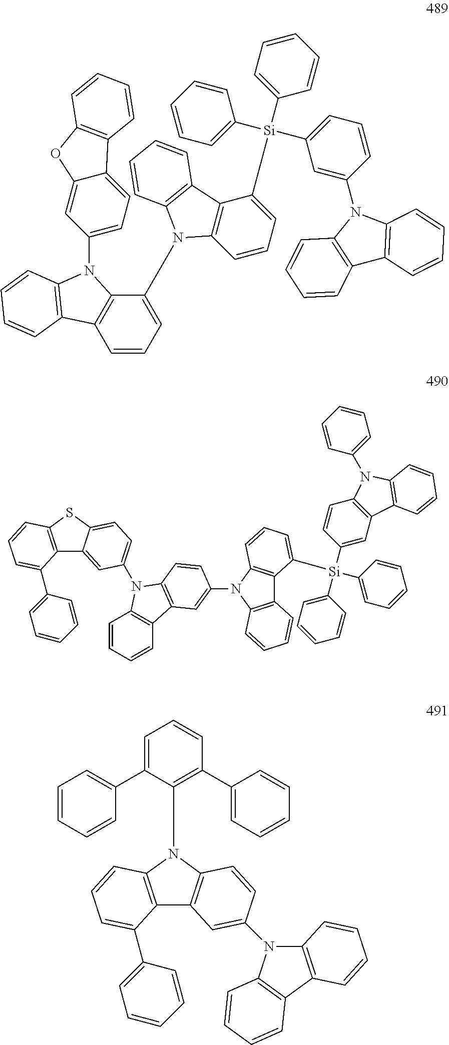 [SMILES: c1ccc([Si](Cc2cccc3c2c2ccccc2n3Cc2cccc3c4ccccc4n(-c4ccc5c(c4)oc4ccccc45)c23)(c2ccccc2)c2cccc(-n3c4ccccc4c4ccccc43)c2)cc1 ; c1ccc(-c2cccc3sc4ccc(-n5c6ccccc6c6cc(-n7c8ccccc8c8c([Si](c9ccccc9)(c9ccccc9)c9ccc%10c(c9)c9ccccc9n%10-c9ccccc9)cccc87)ccc65)cc4c23)cc1 ; c1ccc(-c2cccc(-c3ccccc3)c2Cn2c3ccc(-n4c5ccccc5c5ccccc54)cc3c3c(-c4ccccc4)cccc32)cc1] 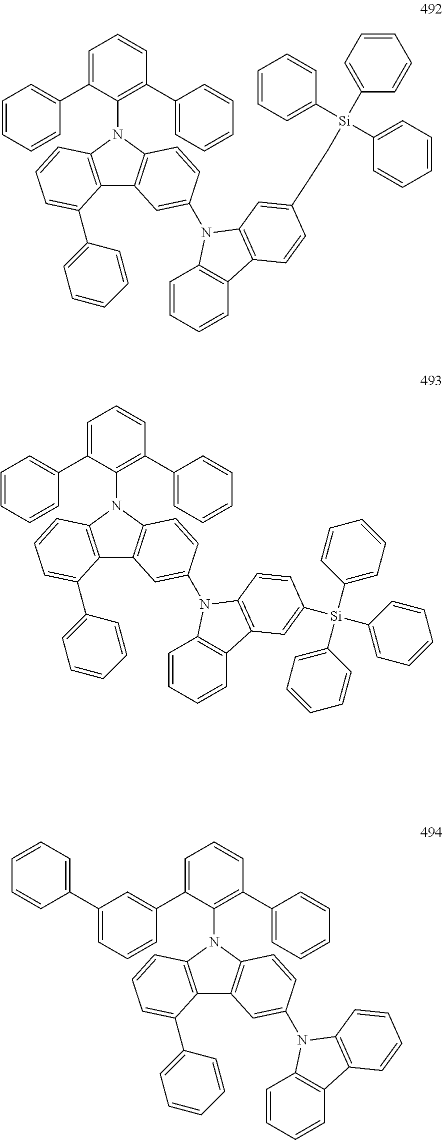 [SMILES: c1ccc(-c2cccc(-c3ccccc3)c2-n2c3ccc(-n4c5ccccc5c5ccc(CC[Si](c6ccccc6)(c6ccccc6)c6ccccc6)cc54)cc3c3c(-c4ccccc4)cccc32)cc1 ; c1ccc(-c2cccc(-c3ccccc3)c2-n2c3ccc(-n4c5ccccc5c5cc([Si](c6ccccc6)(c6ccccc6)c6ccccc6)ccc54)cc3c3c(-c4ccccc4)cccc32)cc1 ; c1ccc(-c2cccc(-c3cccc(-c4ccccc4)c3-n3c4ccc(-n5c6ccccc6c6ccccc65)cc4c4c(-c5ccccc5)cccc43)c2)cc1] 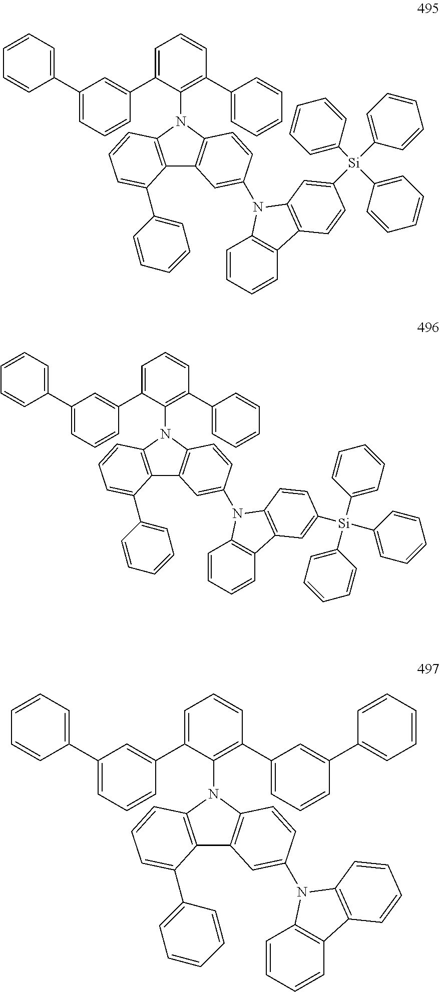 [SMILES: c1ccc(-c2cccc(-c3cccc(-c4ccccc4)c3-n3c4ccc(-n5c6ccccc6c6ccc([Si](c7ccccc7)(c7ccccc7)c7ccccc7)cc65)cc4c4c(-c5ccccc5)cccc43)c2)cc1 ; c1ccc(-c2cccc(-c3cccc(-c4ccccc4)c3-n3c4ccc(-n5c6ccccc6c6cc([Si](c7ccccc7)(c7ccccc7)c7ccccc7)ccc65)cc4c4c(-c5ccccc5)cccc43)c2)cc1 ; c1ccc(-c2cccc(-c3cccc(-c4cccc(-c5ccccc5)c4)c3-n3c4ccc(-n5c6ccccc6c6ccccc65)cc4c4c(-c5ccccc5)cccc43)c2)cc1] 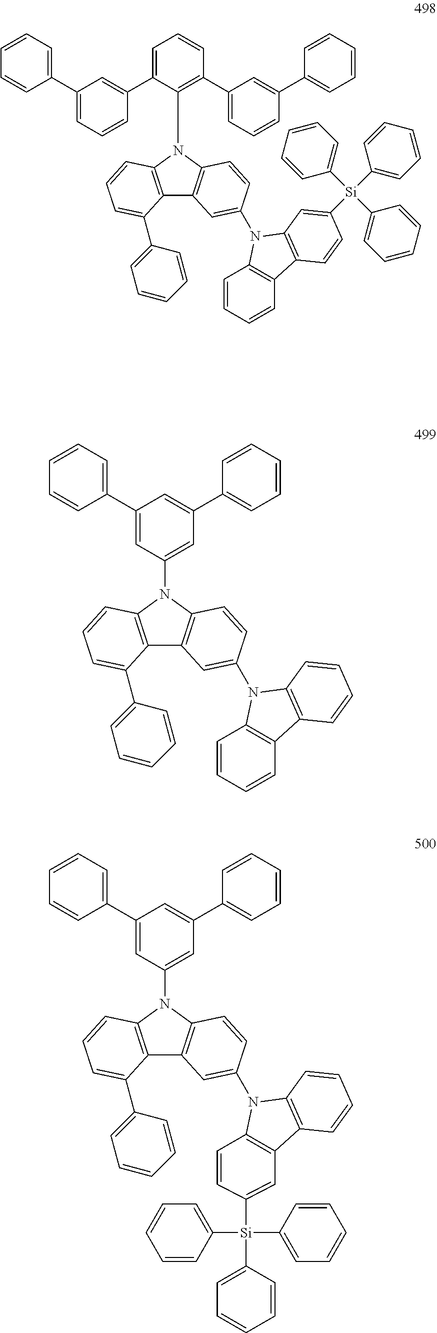 [SMILES: c1ccc(-c2cccc(-c3cccc(-c4cccc(-c5ccccc5)c4)c3Cn3c4ccc(-n5c6ccccc6c6ccc([Si](c7ccccc7)(c7ccccc7)c7ccccc7)cc65)cc4c4c(-c5ccccc5)cccc43)c2)cc1 ; c1ccc(-c2cc(-c3ccccc3)cc(-n3c4ccc(-n5c6ccccc6c6ccccc65)cc4c4c(-c5ccccc5)cccc43)c2)cc1 ; c1ccc(-c2cc(-c3ccccc3)cc(-n3c4ccc(-n5c6ccccc6c6cc([Si](c7ccccc7)(c7ccccc7)c7ccccc7)ccc65)cc4c4c(-c5ccccc5)cccc43)c2)cc1] 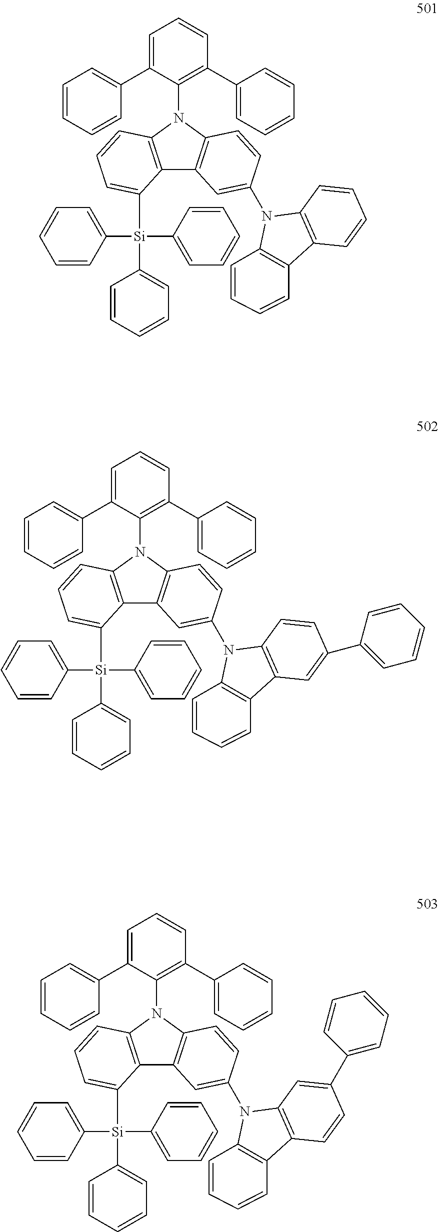 [SMILES: c1ccc(-c2cccc(-c3ccccc3)c2-n2c3ccc(-n4c5ccccc5c5ccccc54)cc3c3c([Si](c4ccccc4)(c4ccccc4)c4ccccc4)cccc32)cc1 ; c1ccc(-c2ccc3c(c2)c2ccccc2n3-c2ccc3c(c2)c2c([Si](c4ccccc4)(c4ccccc4)c4ccccc4)cccc2n3-c2c(-c3ccccc3)cccc2-c2ccccc2)cc1 ; c1ccc(-c2ccc3c4ccccc4n(-c4ccc5c(c4)c4c([Si](c6ccccc6)(c6ccccc6)c6ccccc6)cccc4n5-c4c(-c5ccccc5)cccc4-c4ccccc4)c3c2)cc1] 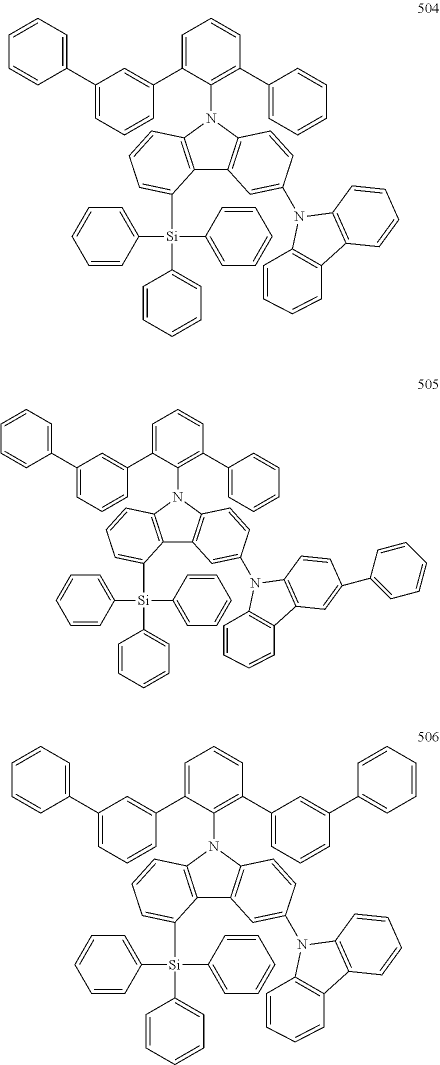 [SMILES: c1ccc(-c2cccc(-c3cccc(-c4ccccc4)c3-n3c4ccc(-n5c6ccccc6c6ccccc65)cc4c4c([Si](c5ccccc5)(c5ccccc5)c5ccccc5)cccc43)c2)cc1 ; c1ccc(-c2cccc(-c3cccc(-c4ccccc4)c3-n3c4ccc(-n5c6ccccc6c6cc(-c7ccccc7)ccc65)cc4c4c([Si](c5ccccc5)(c5ccccc5)c5ccccc5)cccc43)c2)cc1 ; c1ccc(-c2cccc(-c3cccc(-c4cccc(-c5ccccc5)c4)c3-n3c4ccc(-n5c6ccccc6c6ccccc65)cc4c4c([Si](c5ccccc5)(c5ccccc5)c5ccccc5)cccc43)c2)cc1] 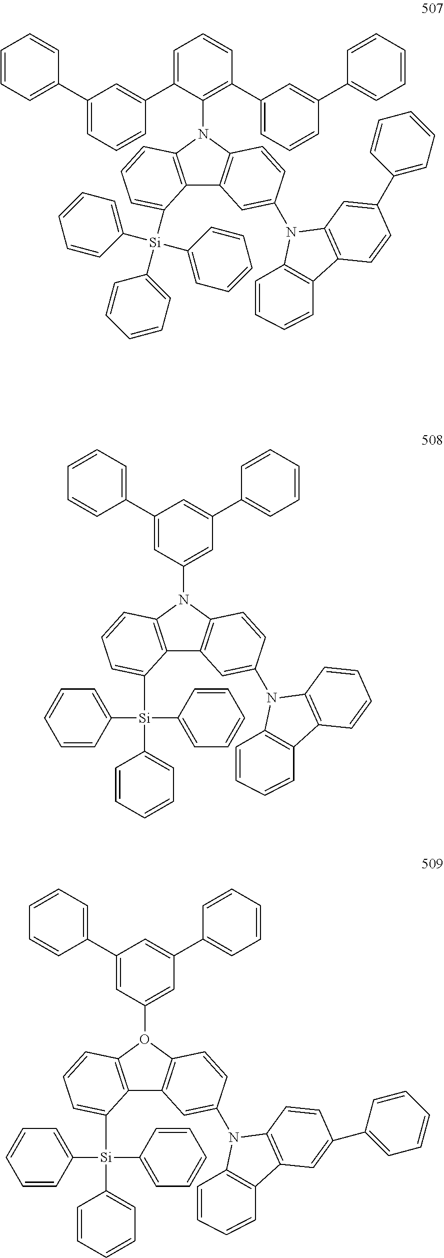 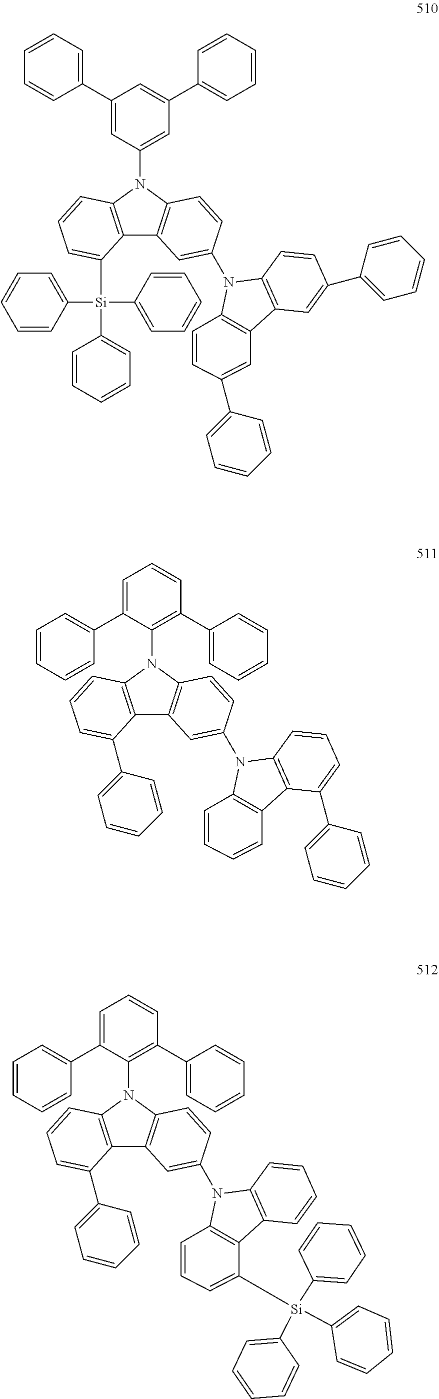 [SMILES: c1ccc(-c2cc(-c3ccccc3)cc(-n3c4ccc(-n5c6ccc(-c7ccccc7)cc6c6cc(-c7ccccc7)ccc65)cc4c4c([Si](c5ccccc5)(c5ccccc5)c5ccccc5)cccc43)c2)cc1 ; c1ccc(-c2cccc(-c3ccccc3)c2-n2c3ccc(-n4c5ccccc5c5c(-c6ccccc6)cccc54)cc3c3c(-c4ccccc4)cccc32)cc1 ; c1ccc(-c2cccc(-c3ccccc3)c2-n2c3ccc(-n4c5ccccc5c5c(C[Si](c6ccccc6)(c6ccccc6)c6ccccc6)cccc54)cc3c3c(-c4ccccc4)cccc32)cc1] 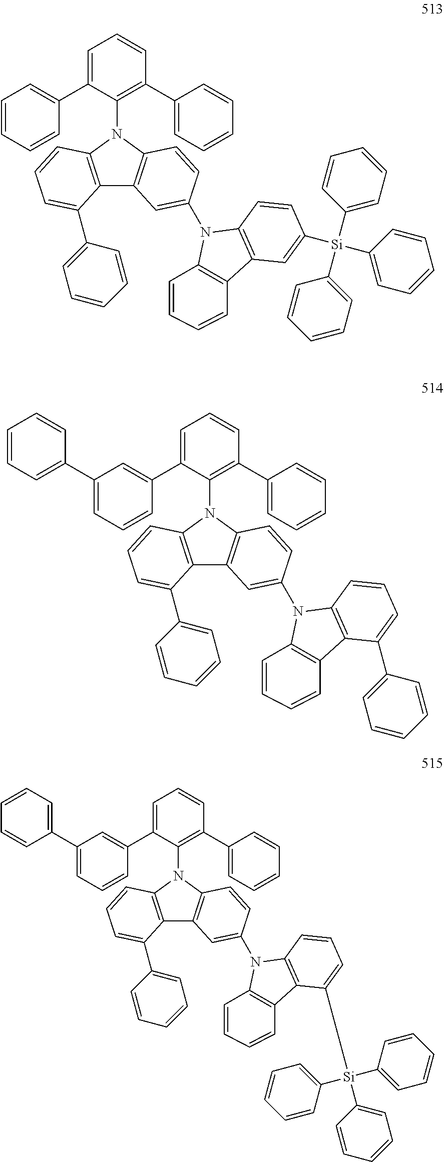 [SMILES: c1ccc(-c2cccc(-c3ccccc3)c2-n2c3ccc(-n4c5ccccc5c5cc([Si](c6ccccc6)(c6ccccc6)c6ccccc6)ccc54)cc3c3c(-c4ccccc4)cccc32)cc1 ; c1ccc(-c2cccc(-c3cccc(-c4ccccc4)c3-n3c4ccc(-n5c6ccccc6c6c(-c7ccccc7)cccc65)cc4c4c(-c5ccccc5)cccc43)c2)cc1 ; c1ccc(-c2cccc(-c3cccc(-c4ccccc4)c3-n3c4ccc(-n5c6ccccc6c6c(CC[Si](c7ccccc7)(c7ccccc7)c7ccccc7)cccc65)cc4c4c(-c5ccccc5)cccc43)c2)cc1] 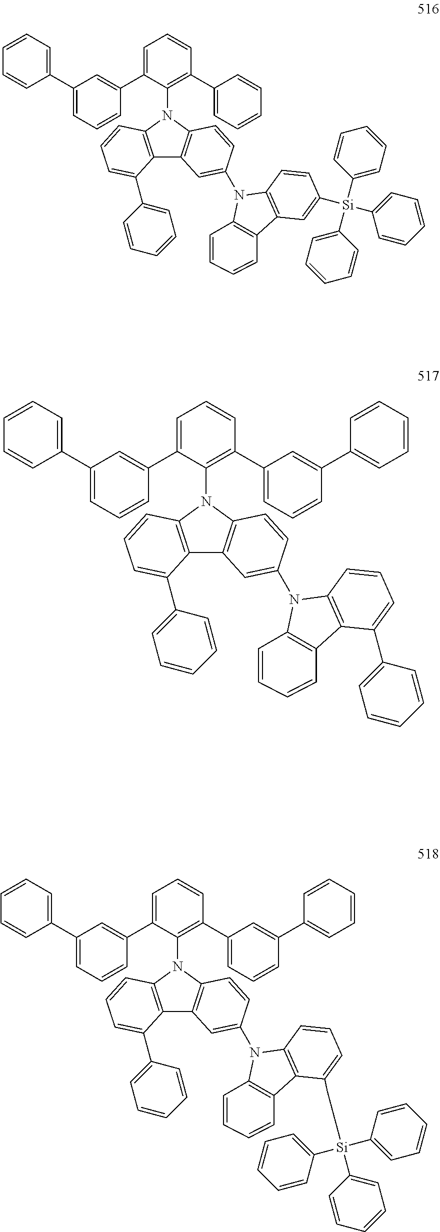 [SMILES: c1ccc(-c2cccc(-c3cccc(-c4ccccc4)c3-n3c4ccc(-n5c6ccccc6c6cc([Si](c7ccccc7)(c7ccccc7)c7ccccc7)ccc65)cc4c4c(-c5ccccc5)cccc43)c2)cc1 ; c1ccc(-c2cccc(-c3cccc(-c4cccc(-c5ccccc5)c4)c3-n3c4ccc(-n5c6ccccc6c6c(-c7ccccc7)cccc65)cc4c4c(-c5ccccc5)cccc43)c2)cc1 ; c1ccc(-c2cccc(-c3cccc(-c4cccc(-c5ccccc5)c4)c3-n3c4ccc(-n5c6ccccc6c6c(CC[Si](c7ccccc7)(c7ccccc7)c7ccccc7)cccc65)cc4c4c(-c5ccccc5)cccc43)c2)cc1] 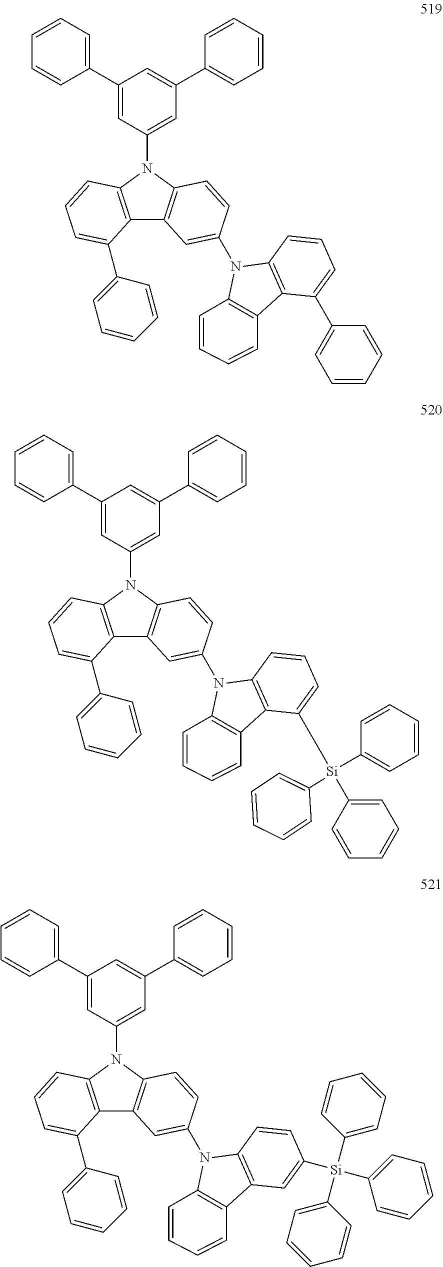 [SMILES: c1ccc(-c2cc(-c3ccccc3)cc(-n3c4ccc(-n5c6ccccc6c6c(-c7ccccc7)cccc65)cc4c4c(-c5ccccc5)cccc43)c2)cc1 ; c1ccc(-c2cc(-c3ccccc3)cc(-n3c4ccc(-n5c6ccccc6c6c(C[Si](c7ccccc7)(c7ccccc7)c7ccccc7)cccc65)cc4c4c(-c5ccccc5)cccc43)c2)cc1 ; c1ccc(-c2cc(-c3ccccc3)cc(-n3c4ccc(-n5c6ccccc6c6cc([Si](c7ccccc7)(c7ccccc7)c7ccccc7)ccc65)cc4c4c(-c5ccccc5)cccc43)c2)cc1] 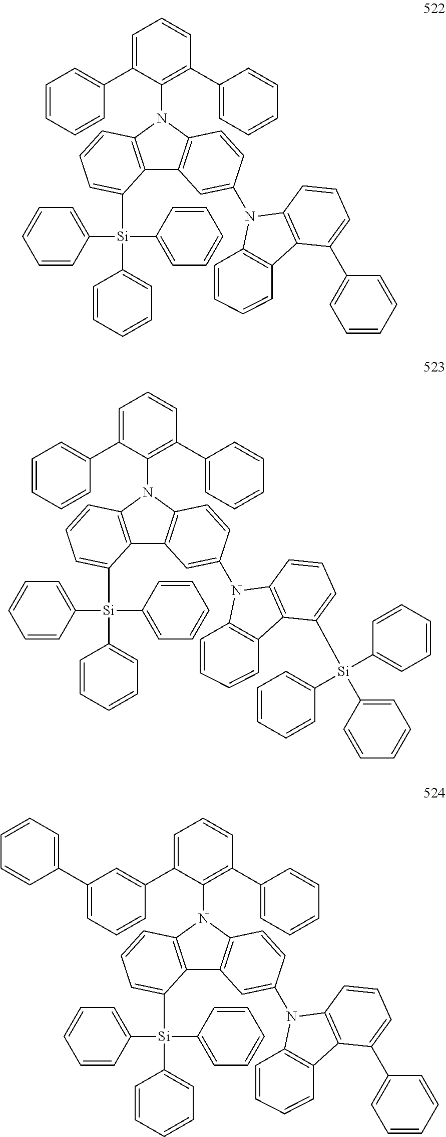 [SMILES: c1ccc(-c2cccc(-c3ccccc3)c2-n2c3ccc(-n4c5ccccc5c5c(-c6ccccc6)cccc54)cc3c3c([Si](c4ccccc4)(c4ccccc4)c4ccccc4)cccc32)cc1 ; c1ccc(-c2cccc(-c3ccccc3)c2-n2c3ccc(-n4c5ccccc5c5c([Si](c6ccccc6)(c6ccccc6)c6ccccc6)cccc54)cc3c3c([Si](c4ccccc4)(c4ccccc4)c4ccccc4)cccc32)cc1 ; c1ccc(-c2cccc(-c3cccc(-c4ccccc4)c3-n3c4ccc(-n5c6ccccc6c6c(-c7ccccc7)cccc65)cc4c4c([Si](c5ccccc5)(c5ccccc5)c5ccccc5)cccc43)c2)cc1] 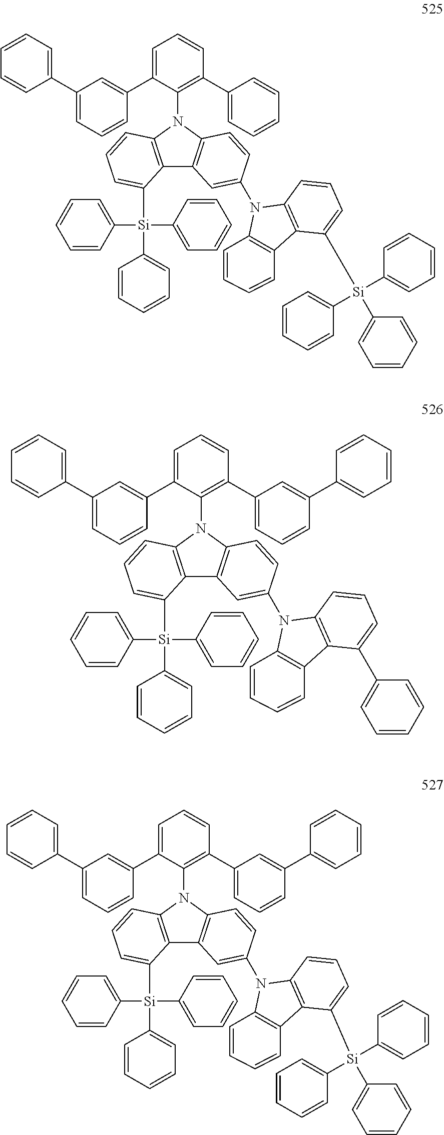 [SMILES: c1ccc(-c2cccc(-c3cccc(-c4ccccc4)c3-n3c4ccc(-n5c6ccccc6c6c(C[Si](c7ccccc7)(c7ccccc7)c7ccccc7)cccc65)cc4c4c([Si](c5ccccc5)(c5ccccc5)c5ccccc5)cccc43)c2)cc1 ; c1ccc(-c2cccc(-c3cccc(-c4cccc(-c5ccccc5)c4)c3-n3c4ccc(-n5c6ccccc6c6c(-c7ccccc7)cccc65)cc4c4c([Si](c5ccccc5)(c5ccccc5)c5ccccc5)cccc43)c2)cc1 ; c1ccc(-c2cccc(-c3cccc(-c4cccc(-c5ccccc5)c4)c3-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)c2)cc1] 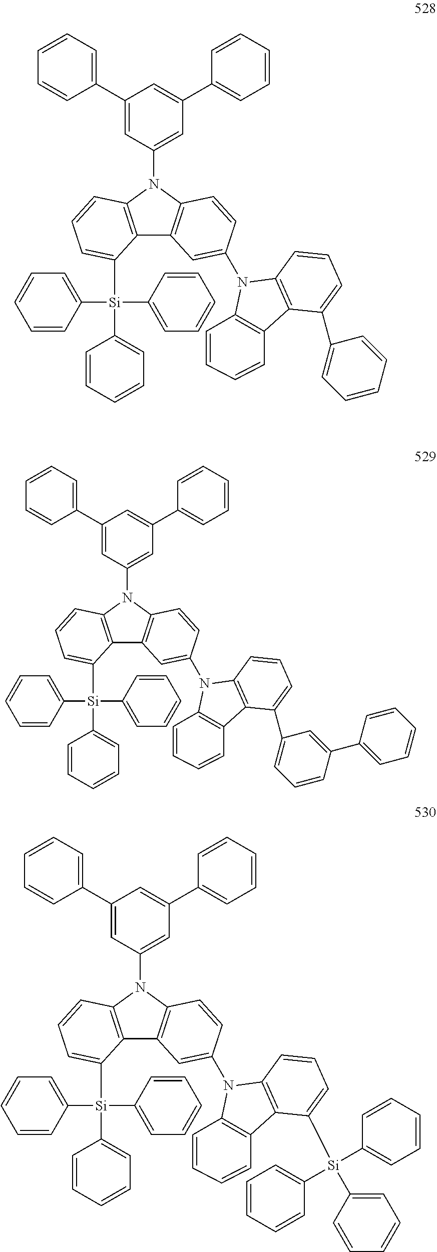 [SMILES: c1ccc(-c2cc(-c3ccccc3)cc(-n3c4ccc(-n5c6ccccc6c6c(-c7ccccc7)cccc65)cc4c4c([Si](c5ccccc5)(c5ccccc5)c5ccccc5)cccc43)c2)cc1 ; c1ccc(-c2cccc(-c3cccc4c3c3ccccc3n4-c3ccc4c(c3)c3c([Si](c5ccccc5)(c5ccccc5)c5ccccc5)cccc3n4-c3cc(-c4ccccc4)cc(-c4ccccc4)c3)c2)cc1 ; c1ccc(-c2cc(-c3ccccc3)cc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)c2)cc1] 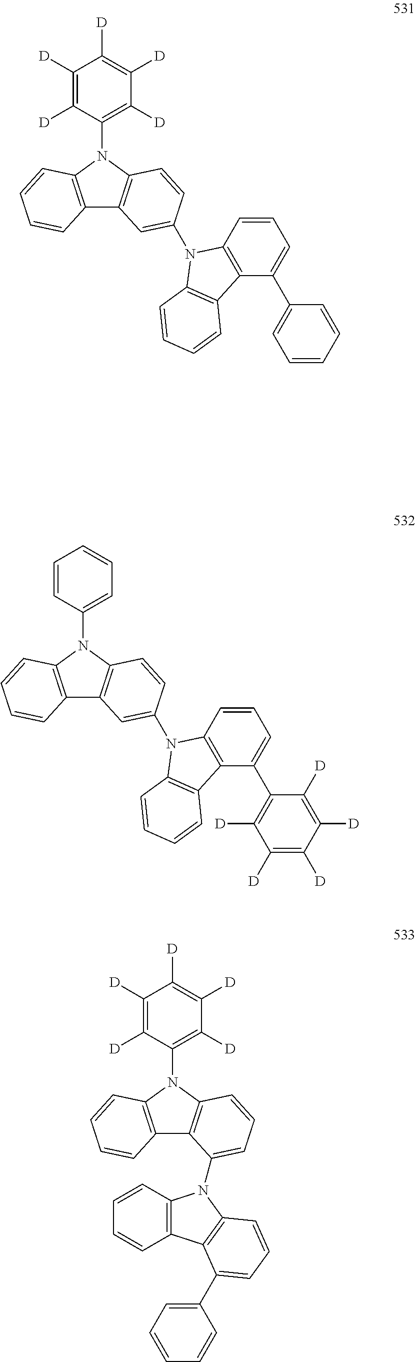 [SMILES: [2H]c1c([2H])c([2H])c(-n2c3ccccc3c3cc(-n4c5ccccc5c5c(-c6ccccc6)cccc54)ccc32)c([2H])c1[2H] ; [2H]c1c([2H])c([2H])c(-c2cccc3c2c2ccccc2n3-c2ccc3c(c2)c2ccccc2n3-c2ccccc2)c([2H])c1[2H] ; [2H]c1c([2H])c([2H])c(-n2c3ccccc3c3c(-n4c5ccccc5c5c(-c6ccccc6)cccc54)cccc32)c([2H])c1[2H]] 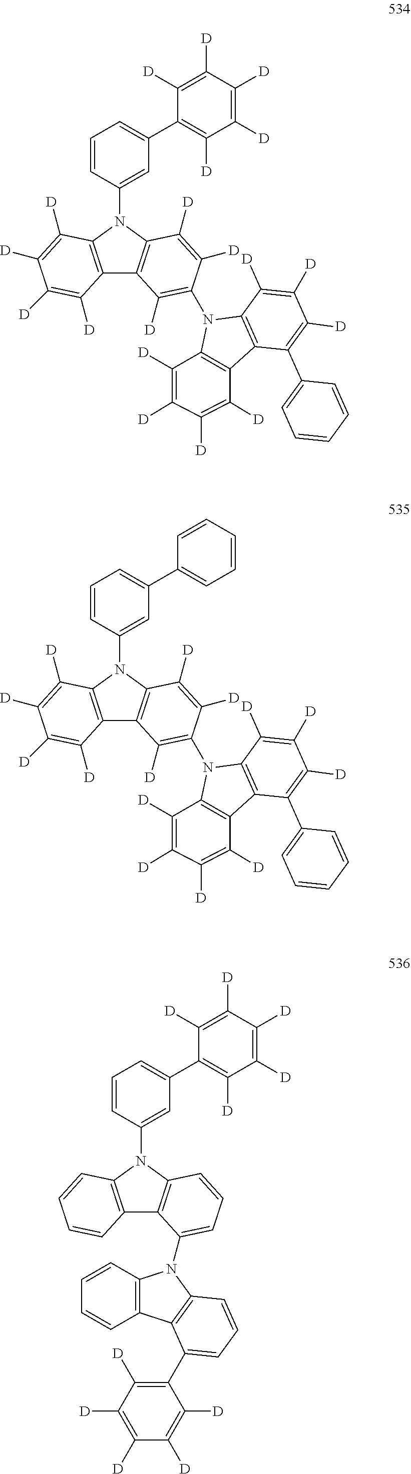 [SMILES: [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c(-c7ccccc7)c([2H])c([2H])c([2H])c65)c([2H])c([2H])c43)c2)c([2H])c1[2H] ; [2H]c1c([2H])c([2H])c2c(c1[2H])c1c([2H])c(-n3c4c([2H])c([2H])c([2H])c([2H])c4c4c(-c5ccccc5)c([2H])c([2H])c([2H])c43)c([2H])c([2H])c1n2-c1cccc(-c2ccccc2)c1 ; [2H]c1c([2H])c([2H])c(-c2cccc(-n3c4ccccc4c4c(-n5c6ccccc6c6c(-c7c([2H])c([2H])c([2H])c([2H])c7[2H])cccc65)cccc43)c2)c([2H])c1[2H]] 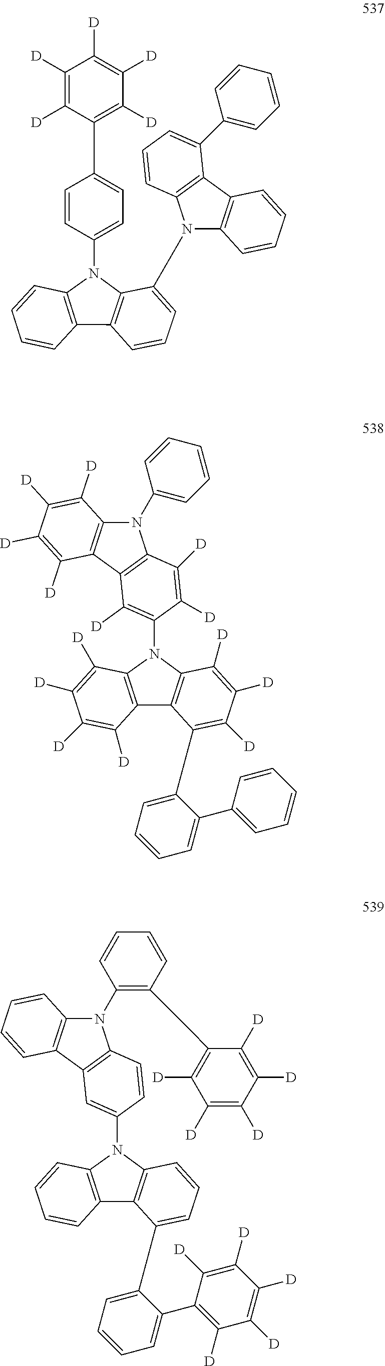 [SMILES: [2H]c1c([2H])c([2H])c(-c2ccc(-n3c4ccccc4c4cccc(Cn5c6ccccc6c6c(-c7ccccc7)cccc65)c43)cc2)c([2H])c1[2H] ; [2H]c1c([2H])c([2H])c2c(c1[2H])c1c([2H])c(-n3c4c([2H])c([2H])c([2H])c([2H])c4c4c(Cc5ccccc5-c5ccccc5)c([2H])c([2H])c([2H])c43)c([2H])c([2H])c1n2-c1ccccc1 ; [2H]c1c([2H])c([2H])c(CCc2ccccc2-n2c3ccccc3c3cc(-n4c5ccccc5c5c(Cc6ccccc6-c6c([2H])c([2H])c([2H])c([2H])c6[2H])cccc54)ccc32)c([2H])c1[2H]] 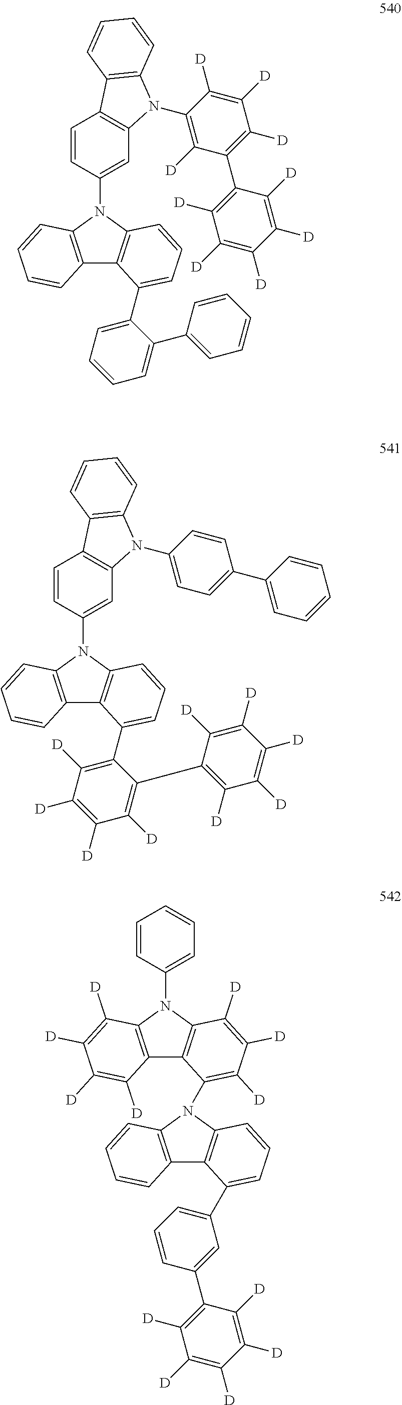 [SMILES: [2H]c1c([2H])c([2H])c(-c2c([2H])c([2H])c([2H])c(-n3c4ccccc4c4ccc(-n5c6ccccc6c6c(-c7ccccc7-c7ccccc7)cccc65)cc43)c2[2H])c([2H])c1[2H] ; [2H]c1c([2H])c([2H])c(Cc2c([2H])c([2H])c([2H])c([2H])c2-c2cccc3c2c2ccccc2n3-c2ccc3c4ccccc4n(-c4ccc(-c5ccccc5)cc4)c3c2)c([2H])c1[2H] ; [2H]c1c([2H])c([2H])c(-c2cccc(-c3cccc4c3c3ccccc3n4-c3c([2H])c([2H])c([2H])c4c3c3c([2H])c([2H])c([2H])c([2H])c3n4-c3ccccc3)c2)c([2H])c1[2H]] 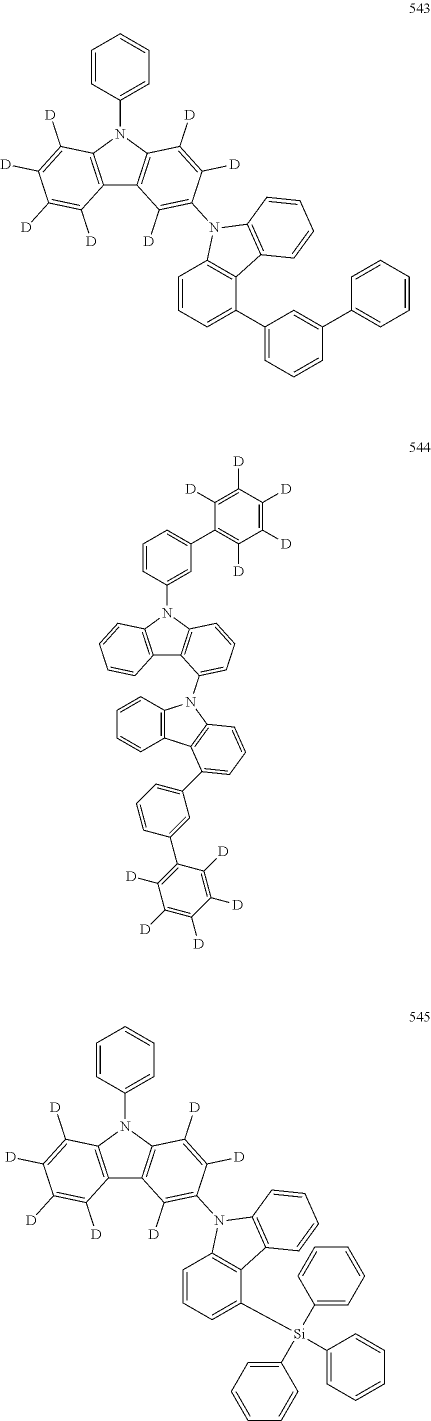 [SMILES: [2H]c1c([2H])c([2H])c2c(c1[2H])c1c([2H])c(-n3c4ccccc4c4c(-c5cccc(-c6ccccc6)c5)cccc43)c([2H])c([2H])c1n2-c1ccccc1 ; [2H]c1c([2H])c([2H])c(-c2cccc(-c3cccc4c3c3ccccc3n4-c3cccc4c3c3ccccc3n4-c3cccc(-c4c([2H])c([2H])c([2H])c([2H])c4[2H])c3)c2)c([2H])c1[2H] ; [2H]c1c([2H])c([2H])c2c(c1[2H])c1c([2H])c(-n3c4ccccc4c4c([Si](c5ccccc5)(c5ccccc5)c5ccccc5)cccc43)c([2H])c([2H])c1n2-c1ccccc1] 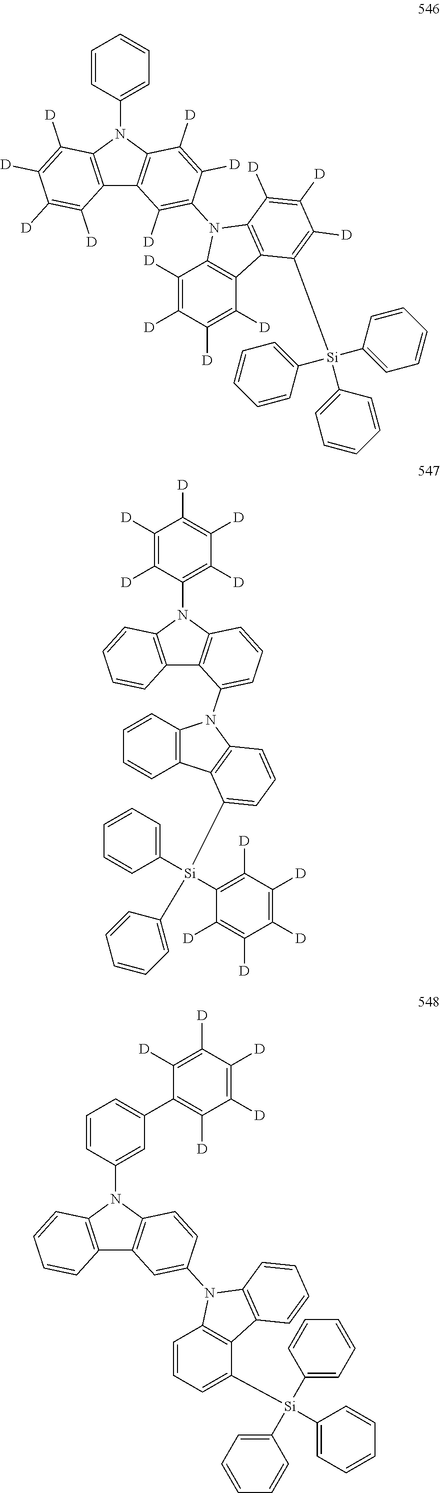 [SMILES: [2H]c1c([2H])c([2H])c2c(c1[2H])c1c([2H])c(-n3c4c([2H])c([2H])c([2H])c([2H])c4c4c([Si](c5ccccc5)(c5ccccc5)c5ccccc5)c([2H])c([2H])c([2H])c43)c([2H])c([2H])c1n2-c1ccccc1 ; [2H]c1c([2H])c([2H])c(-n2c3ccccc3c3c(-n4c5ccccc5c5c([Si](c6ccccc6)(c6ccccc6)c6c([2H])c([2H])c([2H])c([2H])c6[2H])cccc54)cccc32)c([2H])c1[2H] ; [2H]c1c([2H])c([2H])c(-c2cccc(-n3c4ccccc4c4cc(-n5c6ccccc6c6c([Si](c7ccccc7)(c7ccccc7)c7ccccc7)cccc65)ccc43)c2)c([2H])c1[2H]] 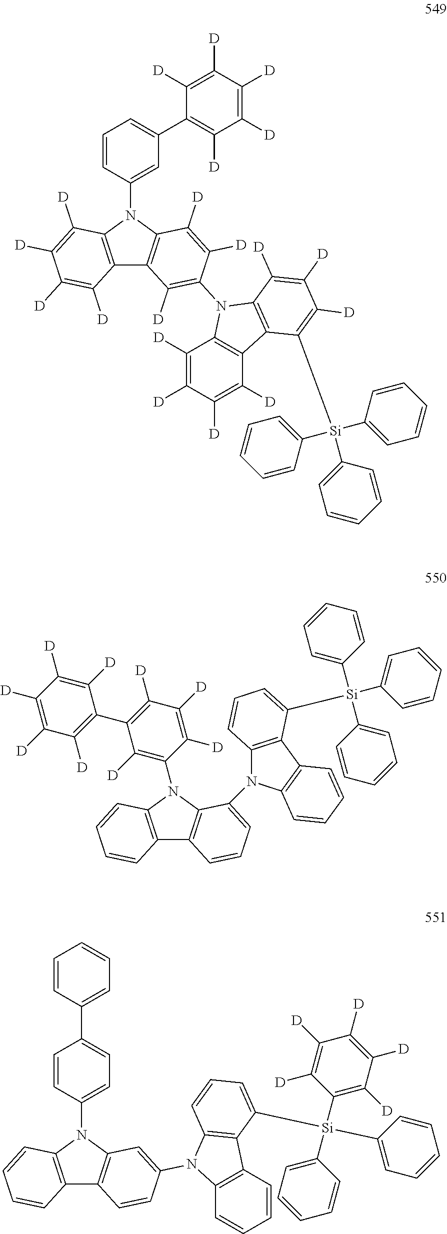 [SMILES: [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c([Si](c7ccccc7)(c7ccccc7)c7ccccc7)c([2H])c([2H])c([2H])c65)c([2H])c([2H])c43)c2)c([2H])c1[2H] ; [2H]c1c([2H])c([2H])c(-c2c([2H])c([2H])c([2H])c(-n3c4ccccc4c4cccc(-n5c6ccccc6c6c([Si](c7ccccc7)(c7ccccc7)c7ccccc7)cccc65)c43)c2[2H])c([2H])c1[2H] ; [2H]c1c([2H])c([2H])c([Si](c2ccccc2)(c2ccccc2)c2cccc3c2c2ccccc2n3-c2ccc3c4ccccc4n(-c4ccc(-c5ccccc5)cc4)c3c2)c([2H])c1[2H]] 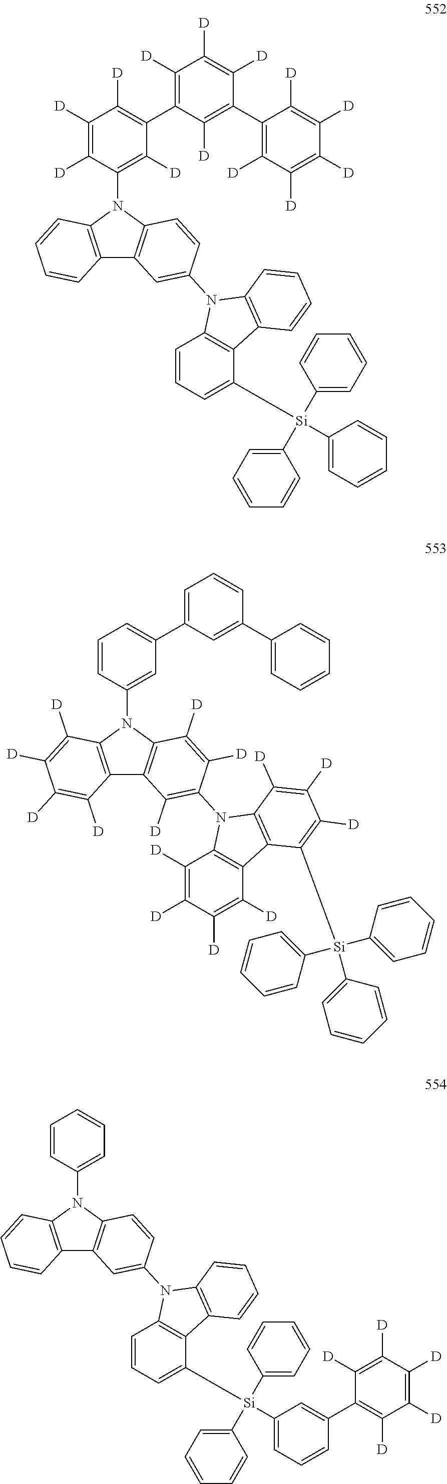 [SMILES: [2H]c1c([2H])c([2H])c(-c2c([2H])c([2H])c([2H])c(-c3c([2H])c([2H])c([2H])c(-n4c5ccccc5c5cc(-n6c7ccccc7c7c([Si](c8ccccc8)(c8ccccc8)c8ccccc8)cccc76)ccc54)c3[2H])c2[2H])c([2H])c1[2H] ; [2H]c1c([2H])c([2H])c2c(c1[2H])c1c([2H])c(-n3c4c([2H])c([2H])c([2H])c([2H])c4c4c([Si](c5ccccc5)(c5ccccc5)c5ccccc5)c([2H])c([2H])c([2H])c43)c([2H])c([2H])c1n2-c1cccc(-c2cccc(-c3ccccc3)c2)c1 ; [2H]c1c([2H])c([2H])c(-c2cccc([Si](c3ccccc3)(c3ccccc3)c3cccc4c3c3ccccc3n4-c3ccc4c(c3)c3ccccc3n4-c3ccccc3)c2)c([2H])c1[2H]] 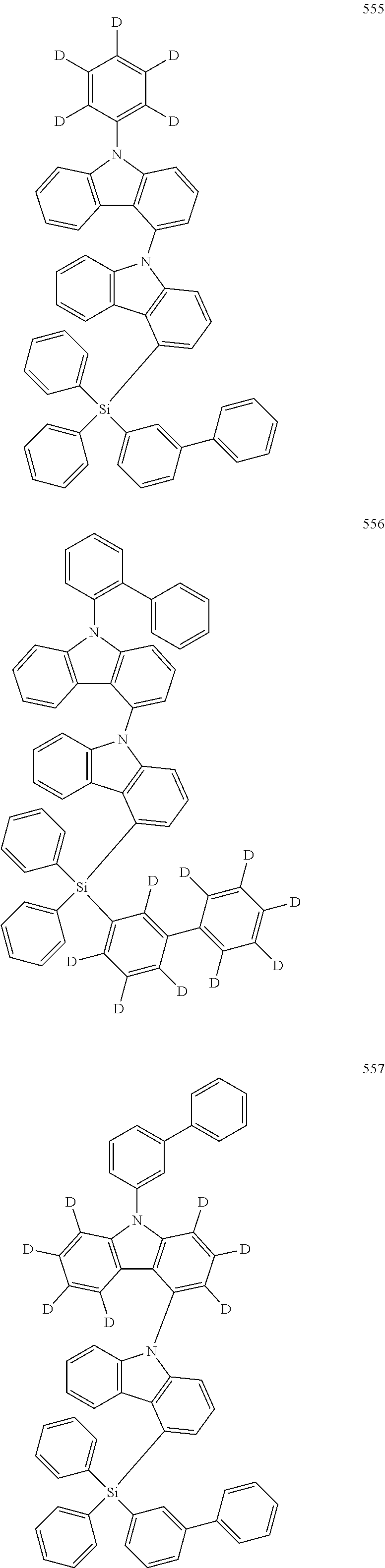 [SMILES: [2H]c1c([2H])c([2H])c(-n2c3ccccc3c3c(-n4c5ccccc5c5c([Si](c6ccccc6)(c6ccccc6)c6cccc(-c7ccccc7)c6)cccc54)cccc32)c([2H])c1[2H] ; [2H]c1c([2H])c([2H])c(-c2c([2H])c([2H])c([2H])c([Si](c3ccccc3)(c3ccccc3)c3cccc4c3c3ccccc3n4-c3cccc4c3c3ccccc3n4-c3ccccc3-c3ccccc3)c2[2H])c([2H])c1[2H] ; [2H]c1c([2H])c([2H])c2c(c1[2H])c1c(-n3c4ccccc4c4c([Si](c5ccccc5)(c5ccccc5)c5cccc(-c6ccccc6)c5)cccc43)c([2H])c([2H])c([2H])c1n2-c1cccc(-c2ccccc2)c1] 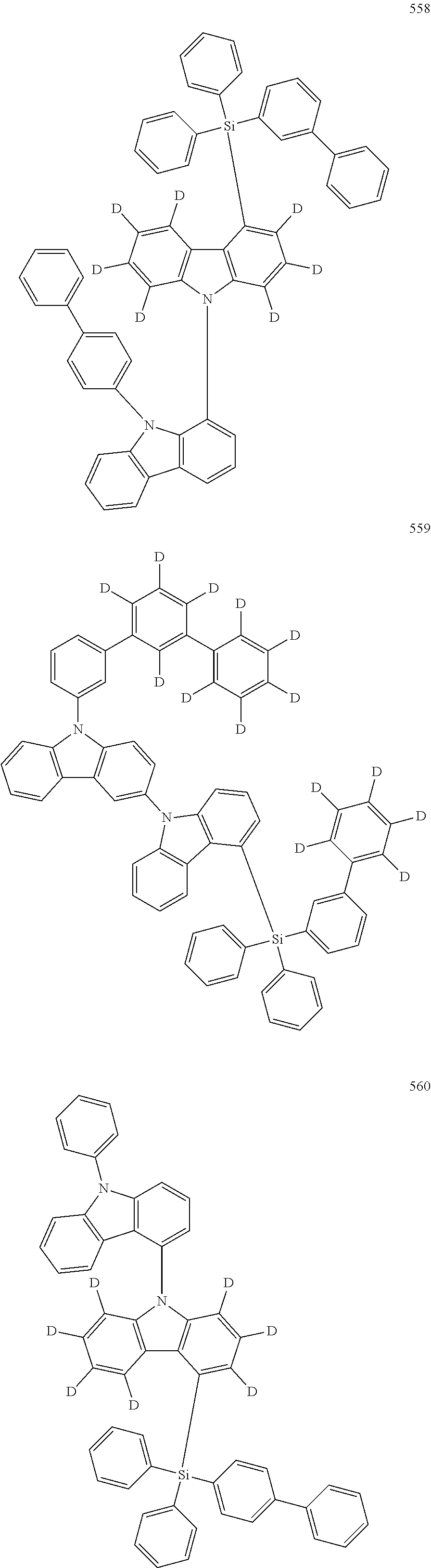 [SMILES: [2H]c1c([2H])c([2H])c2c(c1[2H])c1c([Si](c3ccccc3)(c3ccccc3)c3cccc(-c4ccccc4)c3)c([2H])c([2H])c([2H])c1n2-c1cccc2c3ccccc3n(-c3ccc(-c4ccccc4)cc3)c12 ; [2H]c1c([2H])c([2H])c(-c2cccc([Si](c3ccccc3)(c3ccccc3)c3cccc4c3c3ccccc3n4-c3ccc4c(c3)c3ccccc3n4-c3cccc(-c4c([2H])c([2H])c([2H])c(-c5c([2H])c([2H])c([2H])c([2H])c5[2H])c4[2H])c3)c2)c([2H])c1[2H] ; [2H]c1c([2H])c([2H])c2c(c1[2H])c1c([Si](c3ccccc3)(c3ccccc3)c3ccc(-c4ccccc4)cc3)c([2H])c([2H])c([2H])c1n2-c1cccc2c1c1ccccc1n2-c1ccccc1] 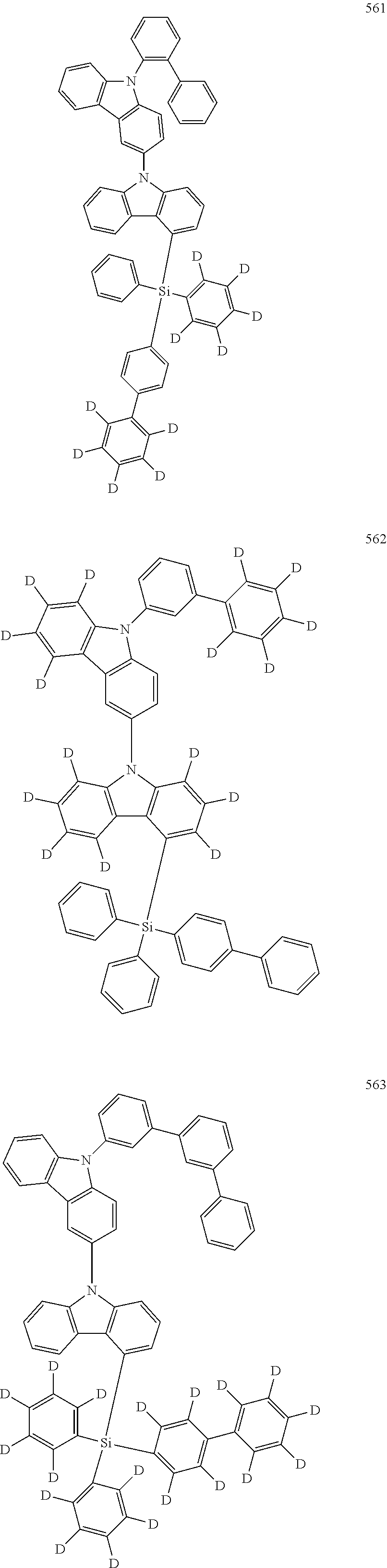 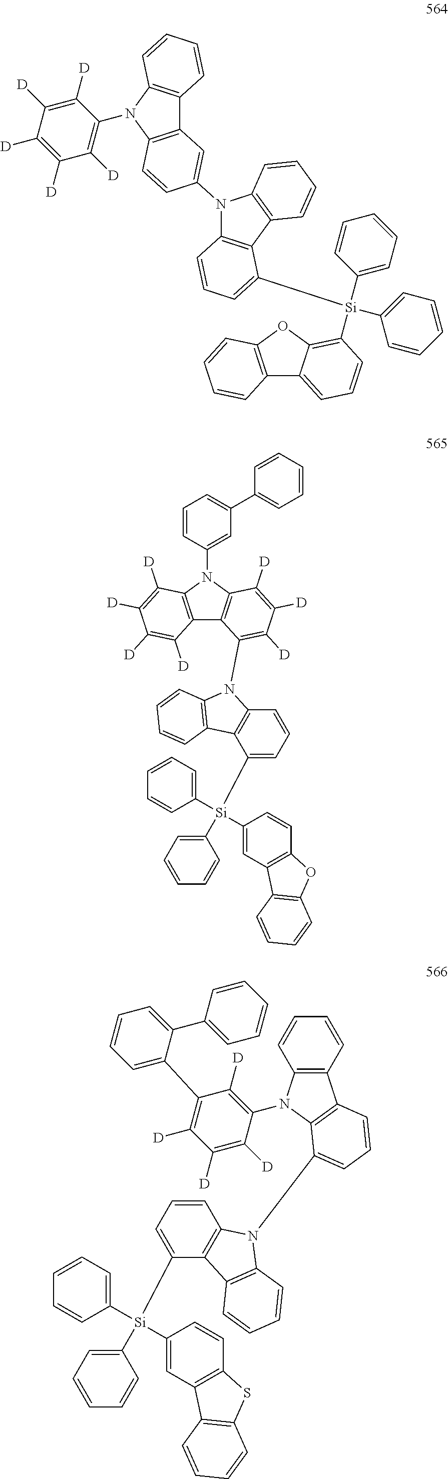 [SMILES: [2H]c1c([2H])c([2H])c(-n2c3ccccc3c3cc(-n4c5ccccc5c5c([Si](c6ccccc6)(c6ccccc6)c6cccc7c6oc6ccccc67)cccc54)ccc32)c([2H])c1[2H] ; [2H]c1c([2H])c([2H])c2c(c1[2H])c1c(-n3c4ccccc4c4c([Si](c5ccccc5)(c5ccccc5)c5ccc6oc7ccccc7c6c5)cccc43)c([2H])c([2H])c([2H])c1n2-c1cccc(-c2ccccc2)c1 ; [2H]c1c([2H])c(-c2ccccc2-c2ccccc2)c([2H])c(-n2c3ccccc3c3cccc(-n4c5ccccc5c5c([Si](c6ccccc6)(c6ccccc6)c6ccc7sc8ccccc8c7c6)cccc54)c32)c1[2H]] 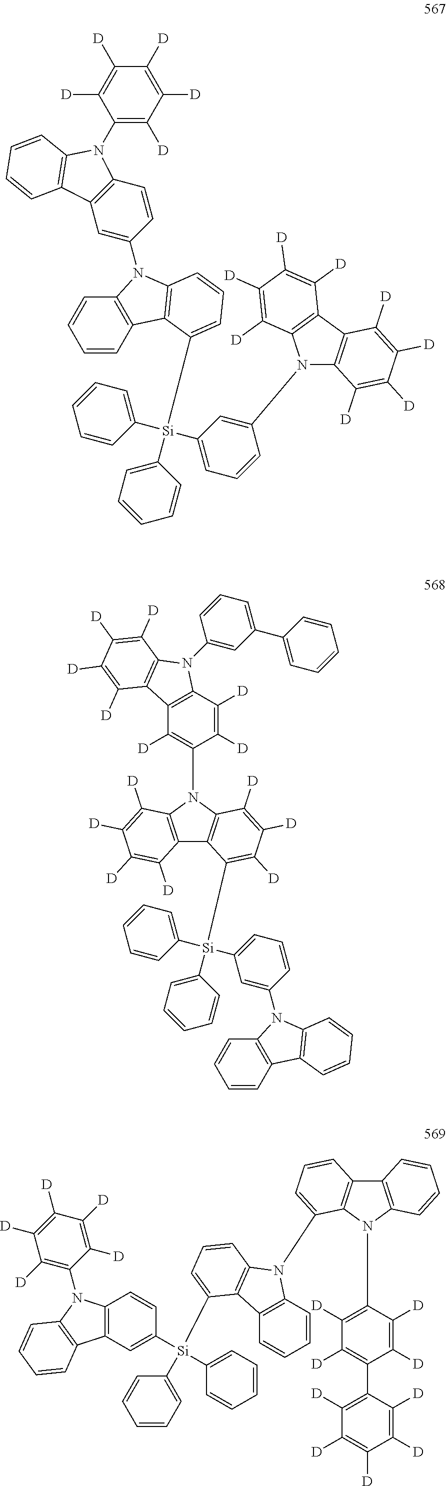 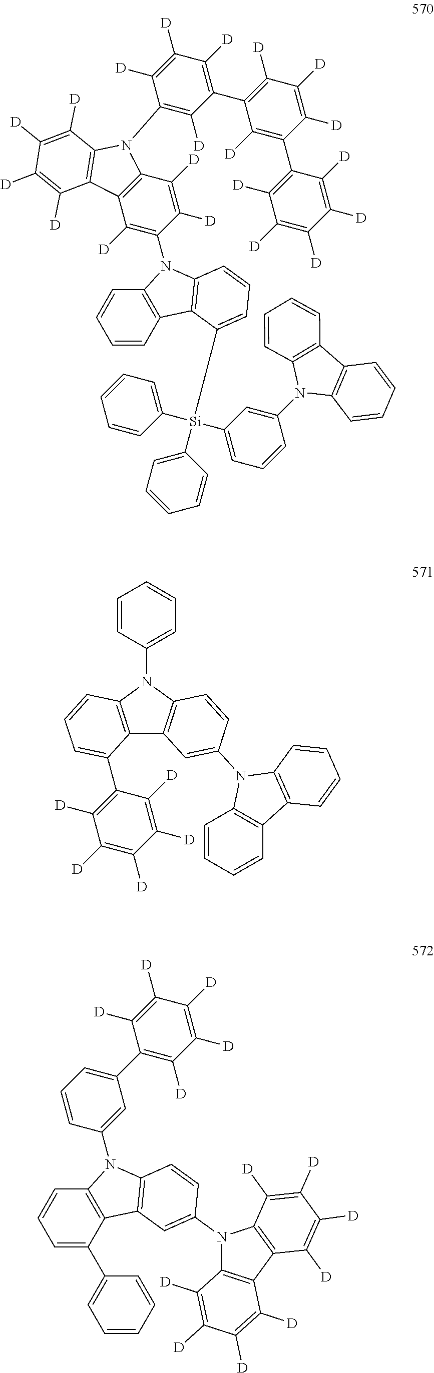 [SMILES: [2H]c1c([2H])c([2H])c(-c2c([2H])c([2H])c([2H])c(-c3c([2H])c([2H])c([2H])c(-n4c5c([2H])c([2H])c([2H])c([2H])c5c5c([2H])c(-n6c7ccccc7c7c([Si](c8ccccc8)(c8ccccc8)c8cccc(-n9c%10ccccc%10c%10ccccc%109)c8)cccc76)c([2H])c([2H])c54)c3[2H])c2[2H])c([2H])c1[2H] ; [2H]c1c([2H])c([2H])c(-c2cccc3c2c2cc(-n4c5ccccc5c5ccccc54)ccc2n3-c2ccccc2)c([2H])c1[2H] ; [2H]c1c([2H])c([2H])c(-c2cccc(-n3c4ccc(-n5c6c([2H])c([2H])c([2H])c([2H])c6c6c([2H])c([2H])c([2H])c([2H])c65)cc4c4c(-c5ccccc5)cccc43)c2)c([2H])c1[2H]] 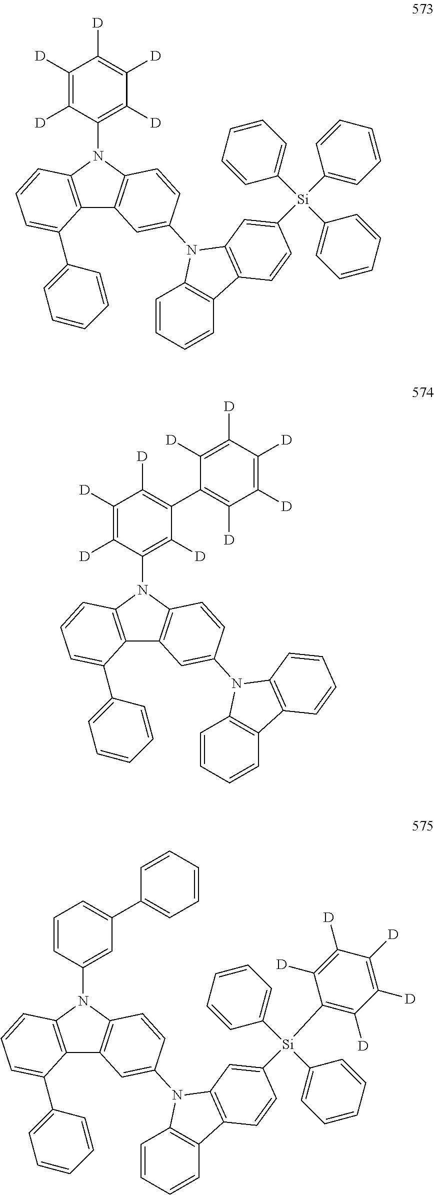 [SMILES: [2H]c1c([2H])c([2H])c(-n2c3ccc(-n4c5ccccc5c5ccc([Si](c6ccccc6)(c6ccccc6)c6ccccc6)cc54)cc3c3c(-c4ccccc4)cccc32)c([2H])c1[2H] ; [2H]c1c([2H])c([2H])c(-c2c([2H])c([2H])c([2H])c(-n3c4ccc(-n5c6ccccc6c6ccccc65)cc4c4c(-c5ccccc5)cccc43)c2[2H])c([2H])c1[2H] ; [2H]c1c([2H])c([2H])c([Si](c2ccccc2)(c2ccccc2)c2ccc3c4ccccc4n(-c4ccc5c(c4)c4c(-c6ccccc6)cccc4n5-c4cccc(-c5ccccc5)c4)c3c2)c([2H])c1[2H]] 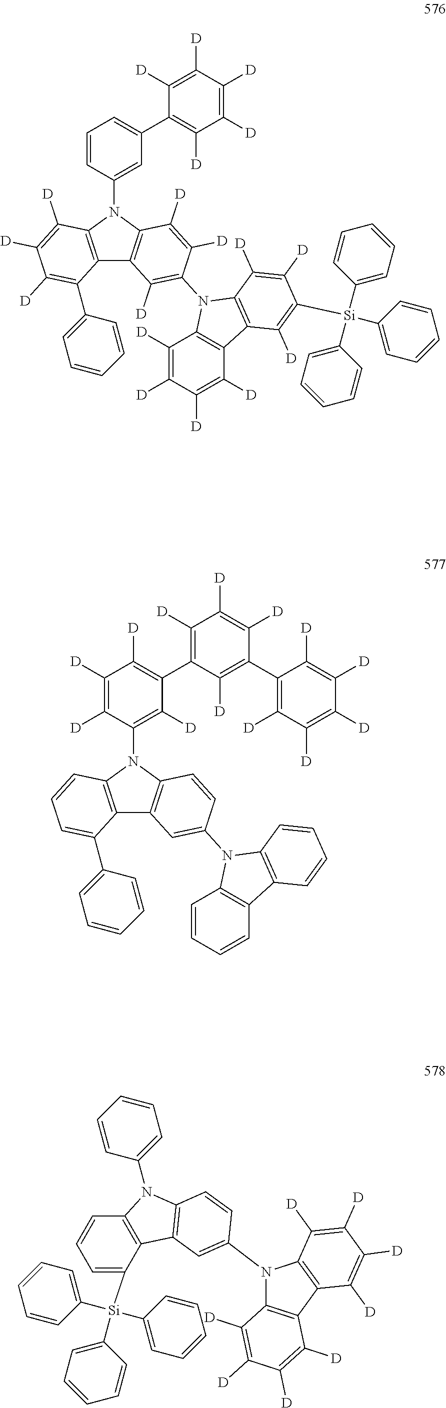 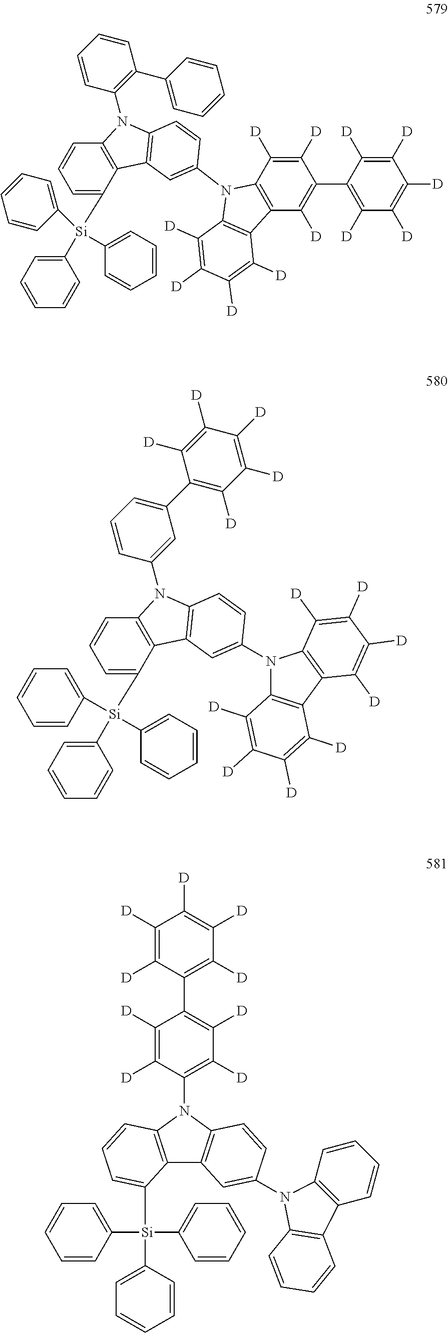 [SMILES: [2H]c1c([2H])c([2H])c(-c2c([2H])c([2H])c3c(c2[2H])c2c([2H])c([2H])c([2H])c([2H])c2n3-c2ccc3c(c2)c2c([Si](c4ccccc4)(c4ccccc4)c4ccccc4)cccc2n3-c2ccccc2-c2ccccc2)c([2H])c1[2H] ; [2H]c1c([2H])c([2H])c(-c2cccc(-n3c4ccc(-n5c6c([2H])c([2H])c([2H])c([2H])c6c6c([2H])c([2H])c([2H])c([2H])c65)cc4c4c([Si](c5ccccc5)(c5ccccc5)c5ccccc5)cccc43)c2)c([2H])c1[2H] ; [2H]c1c([2H])c([2H])c(-c2c([2H])c([2H])c(-n3c4ccc(-n5c6ccccc6c6ccccc65)cc4c4c([Si](c5ccccc5)(c5ccccc5)c5ccccc5)cccc43)c([2H])c2[2H])c([2H])c1[2H]] 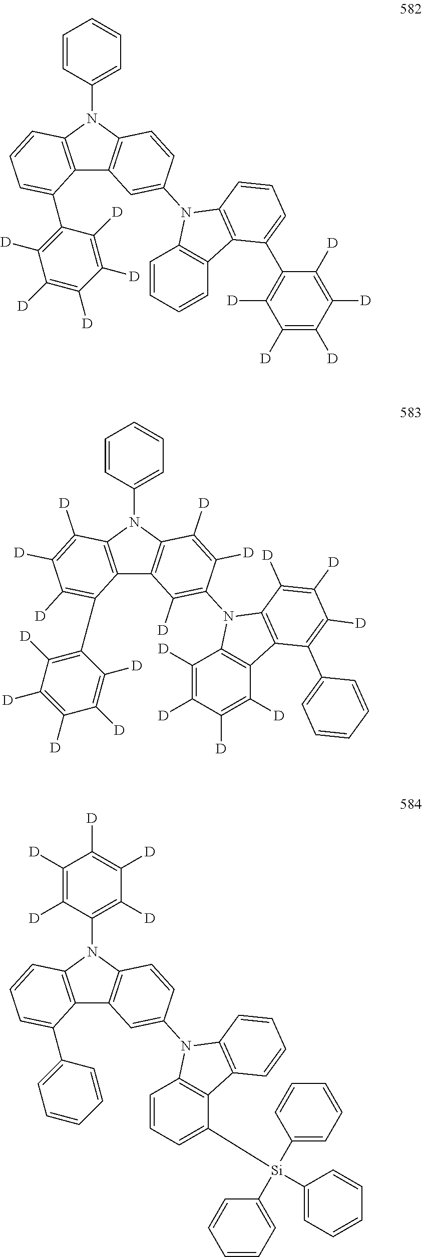 [SMILES: [2H]c1c([2H])c([2H])c(-c2cccc3c2c2ccccc2n3-c2ccc3c(c2)c2c(-c4c([2H])c([2H])c([2H])c([2H])c4[2H])cccc2n3-c2ccccc2)c([2H])c1[2H] ; [2H]c1c([2H])c([2H])c(-c2c([2H])c([2H])c([2H])c3c2c2c([2H])c(-n4c5c([2H])c([2H])c([2H])c([2H])c5c5c(-c6ccccc6)c([2H])c([2H])c([2H])c54)c([2H])c([2H])c2n3-c2ccccc2)c([2H])c1[2H] ; [2H]c1c([2H])c([2H])c(-n2c3ccc(-n4c5ccccc5c5c([Si](c6ccccc6)(c6ccccc6)c6ccccc6)cccc54)cc3c3c(-c4ccccc4)cccc32)c([2H])c1[2H]] 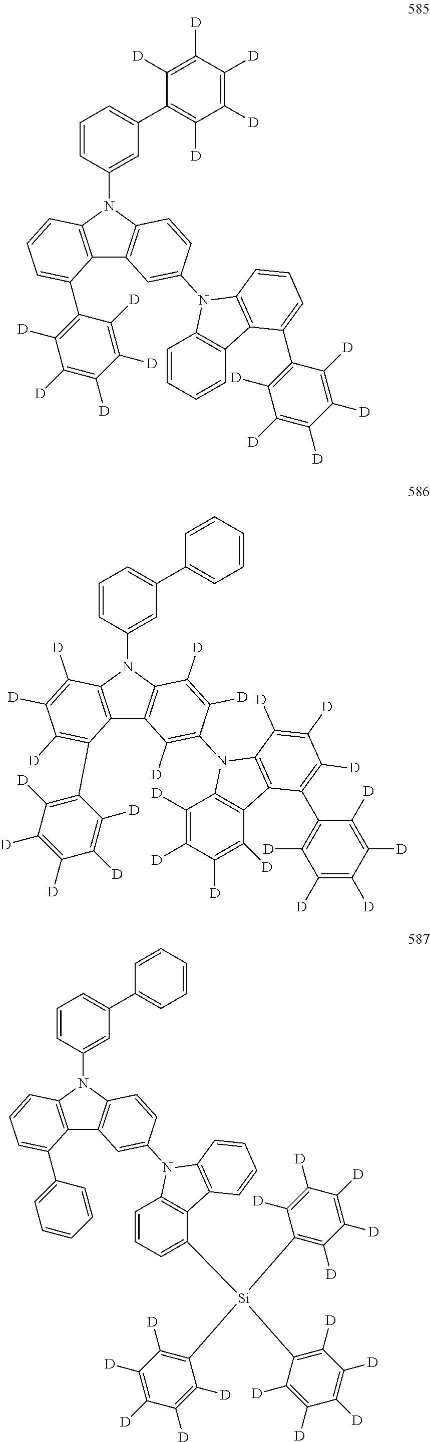 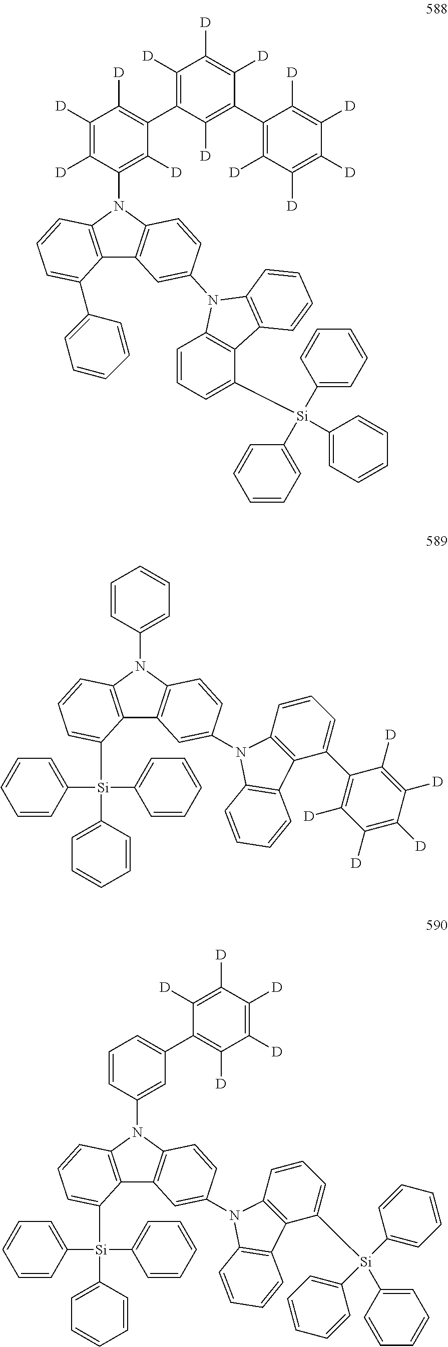 [SMILES: [2H]c1c([2H])c([2H])c(-c2c([2H])c([2H])c([2H])c(-c3c([2H])c([2H])c([2H])c(-n4c5ccc(-n6c7ccccc7c7c([Si](c8ccccc8)(c8ccccc8)c8ccccc8)cccc76)cc5c5c(-c6ccccc6)cccc54)c3[2H])c2[2H])c([2H])c1[2H] ; [2H]c1c([2H])c([2H])c(-c2cccc3c2c2ccccc2n3-c2ccc3c(c2)c2c([Si](c4ccccc4)(c4ccccc4)c4ccccc4)cccc2n3-c2ccccc2)c([2H])c1[2H] ; [2H]c1c([2H])c([2H])c(-c2cccc(-n3c4ccc(-n5c6ccccc6c6c([Si](c7ccccc7)(c7ccccc7)c7ccccc7)cccc65)cc4c4c([Si](c5ccccc5)(c5ccccc5)c5ccccc5)cccc43)c2)c([2H])c1[2H]] 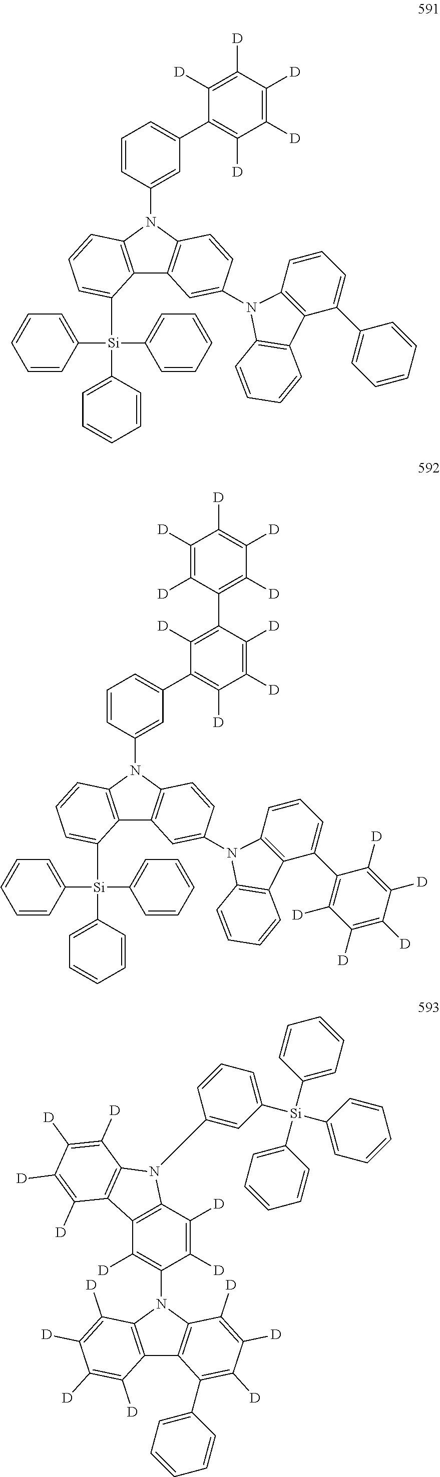 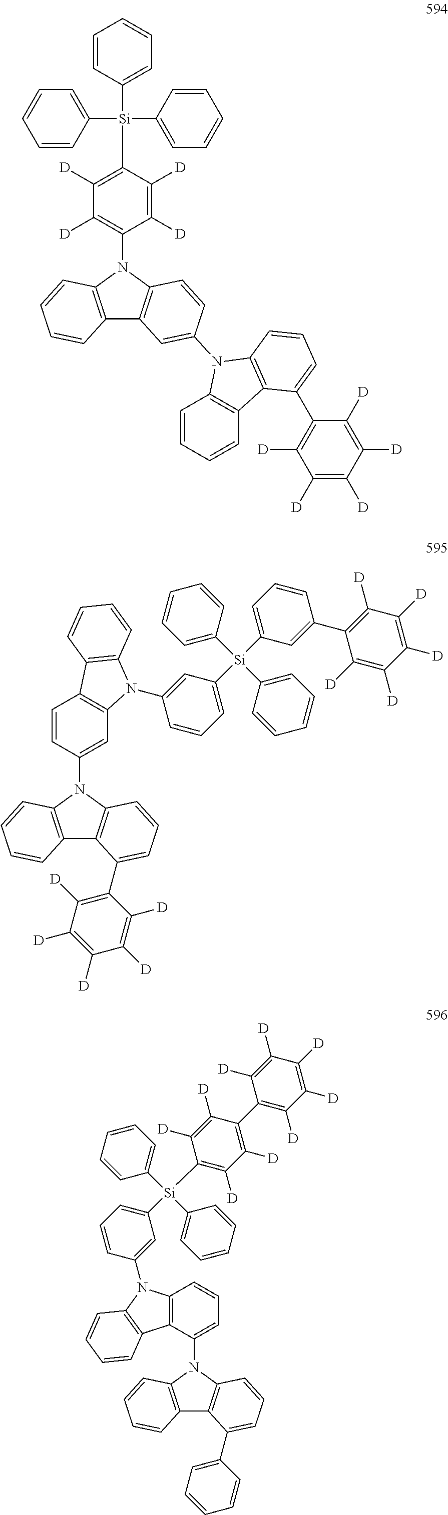 [SMILES: [2H]c1c([2H])c([2H])c(-c2cccc3c2c2ccccc2n3-c2ccc3c(c2)c2ccccc2n3-c2c([2H])c([2H])c([Si](c3ccccc3)(c3ccccc3)c3ccccc3)c([2H])c2[2H])c([2H])c1[2H] ; [2H]c1c([2H])c([2H])c(-c2cccc([Si](c3ccccc3)(c3ccccc3)c3cccc(-n4c5ccccc5c5ccc(-n6c7ccccc7c7c(-c8c([2H])c([2H])c([2H])c([2H])c8[2H])cccc76)cc54)c3)c2)c([2H])c1[2H] ; [2H]c1c([2H])c([2H])c(-c2c([2H])c([2H])c([Si](c3ccccc3)(c3ccccc3)c3cccc(-n4c5ccccc5c5c(-n6c7ccccc7c7c(-c8ccccc8)cccc76)cccc54)c3)c([2H])c2[2H])c([2H])c1[2H]] 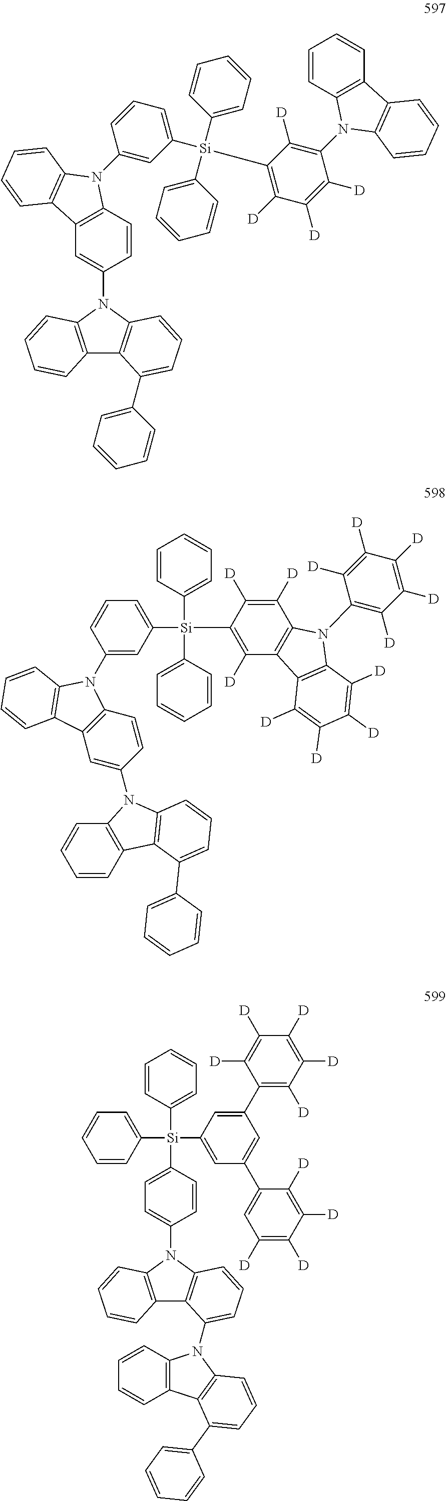 [SMILES: [2H]c1c([2H])c(-n2c3ccccc3c3ccccc32)c([2H])c([Si](c2ccccc2)(c2ccccc2)c2cccc(-n3c4ccccc4c4cc(-n5c6ccccc6c6c(-c7ccccc7)cccc65)ccc43)c2)c1[2H] ; [2H]c1c([2H])c([2H])c(-n2c3c([2H])c([2H])c([2H])c([2H])c3c3c([2H])c([Si](c4ccccc4)(c4ccccc4)c4cccc(-n5c6ccccc6c6cc(-n7c8ccccc8c8c(-c9ccccc9)cccc87)ccc65)c4)c([2H])c([2H])c32)c([2H])c1[2H] ; [2H]c1cc(-c2cc(-c3c([2H])c([2H])c([2H])c([2H])c3[2H])cc([Si](c3ccccc3)(c3ccccc3)c3ccc(-n4c5ccccc5c5c(-n6c7ccccc7c7c(-c8ccccc8)cccc76)cccc54)cc3)c2)c([2H])c([2H])c1[2H]] 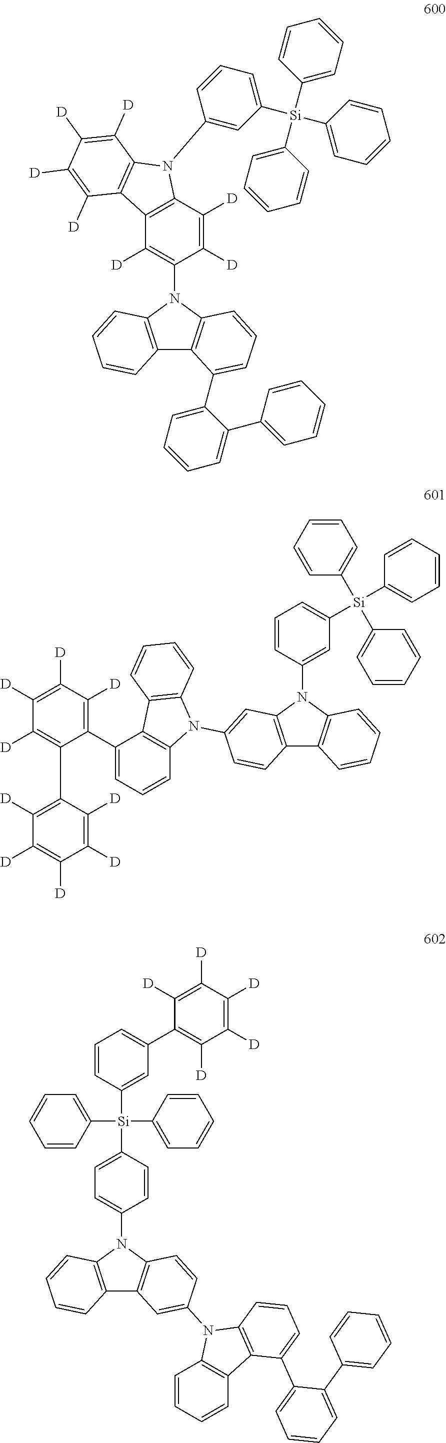 [SMILES: [2H]c1c([2H])c([2H])c2c(c1[2H])c1c([2H])c(-n3c4ccccc4c4c(-c5ccccc5-c5ccccc5)cccc43)c([2H])c([2H])c1n2-c1cccc([Si](c2ccccc2)(c2ccccc2)c2ccccc2)c1 ; [2H]c1c([2H])c([2H])c(-c2c([2H])c([2H])c([2H])c([2H])c2-c2cccc3c2c2ccccc2n3-c2ccc3c4ccccc4n(-c4cccc([Si](c5ccccc5)(c5ccccc5)c5ccccc5)c4)c3c2)c([2H])c1[2H] ; [2H]c1c([2H])c([2H])c(-c2cccc([Si](c3ccccc3)(c3ccccc3)c3ccc(-n4c5ccccc5c5cc(-n6c7ccccc7c7c(-c8ccccc8-c8ccccc8)cccc76)ccc54)cc3)c2)c([2H])c1[2H]] 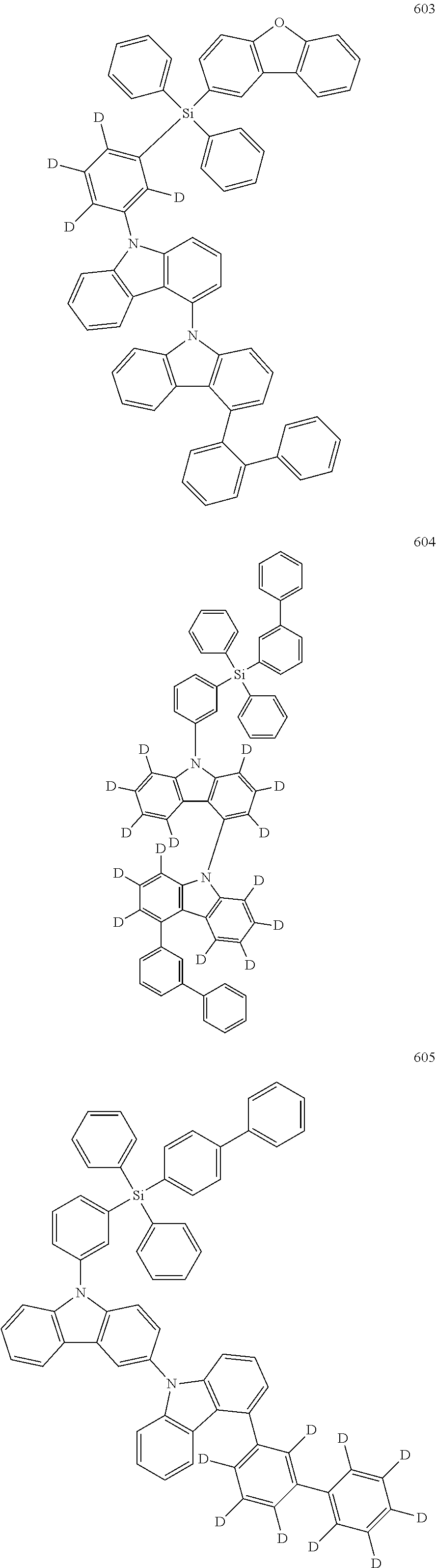 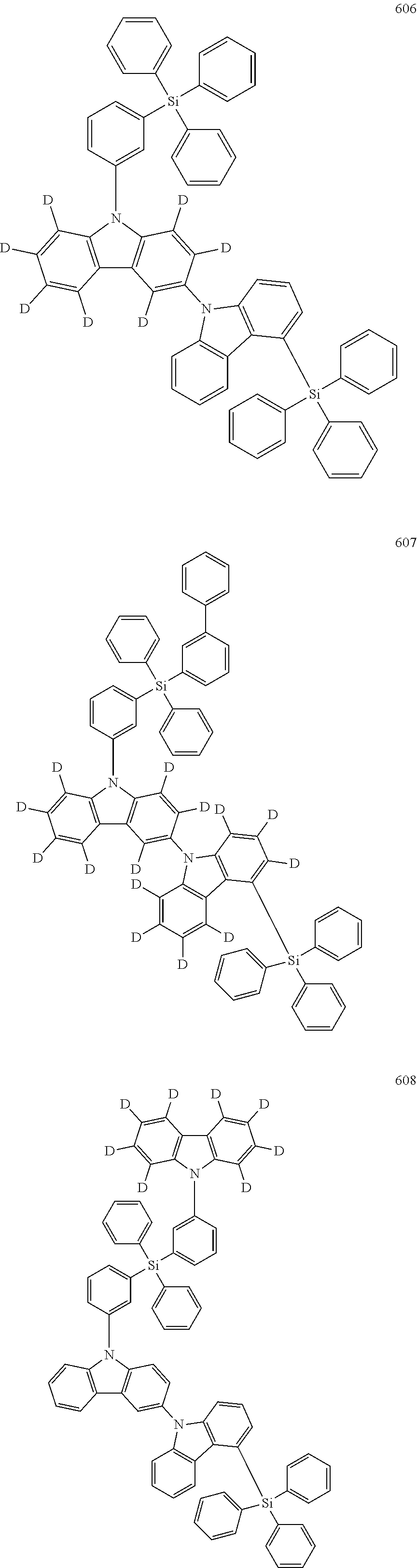 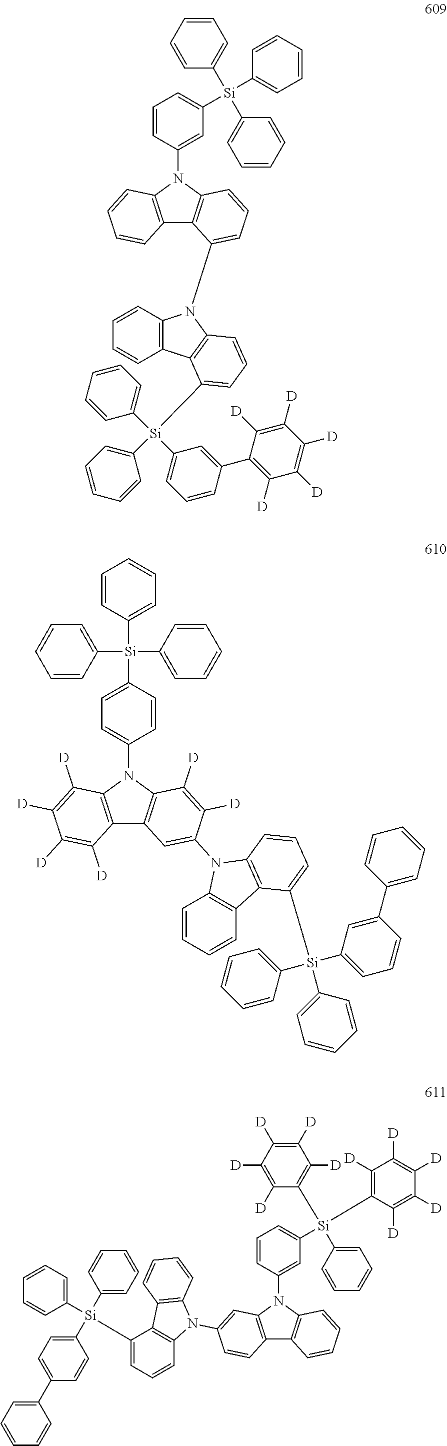 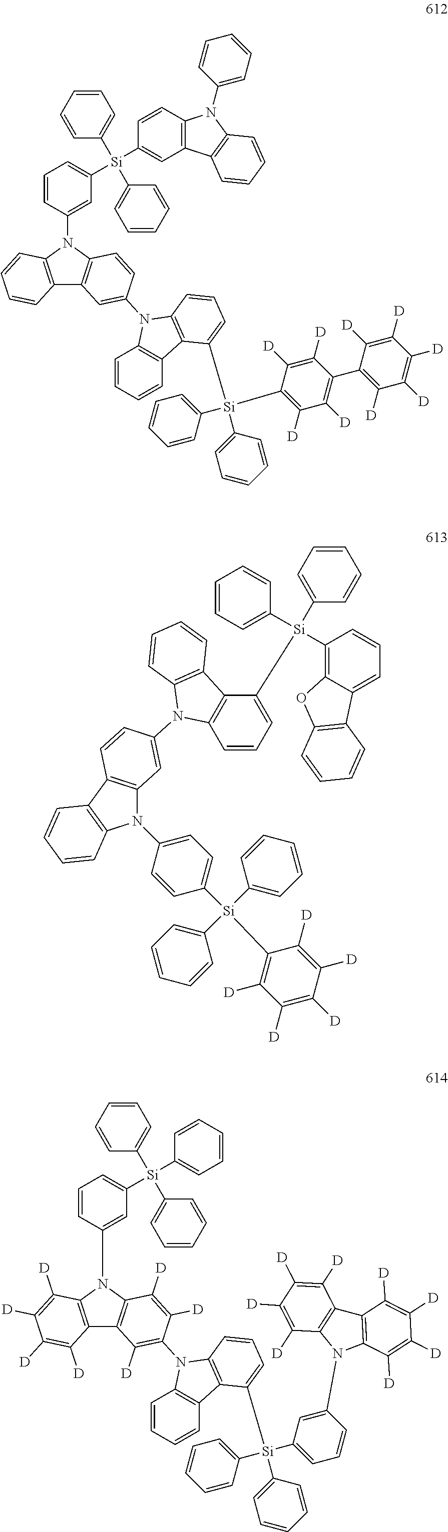 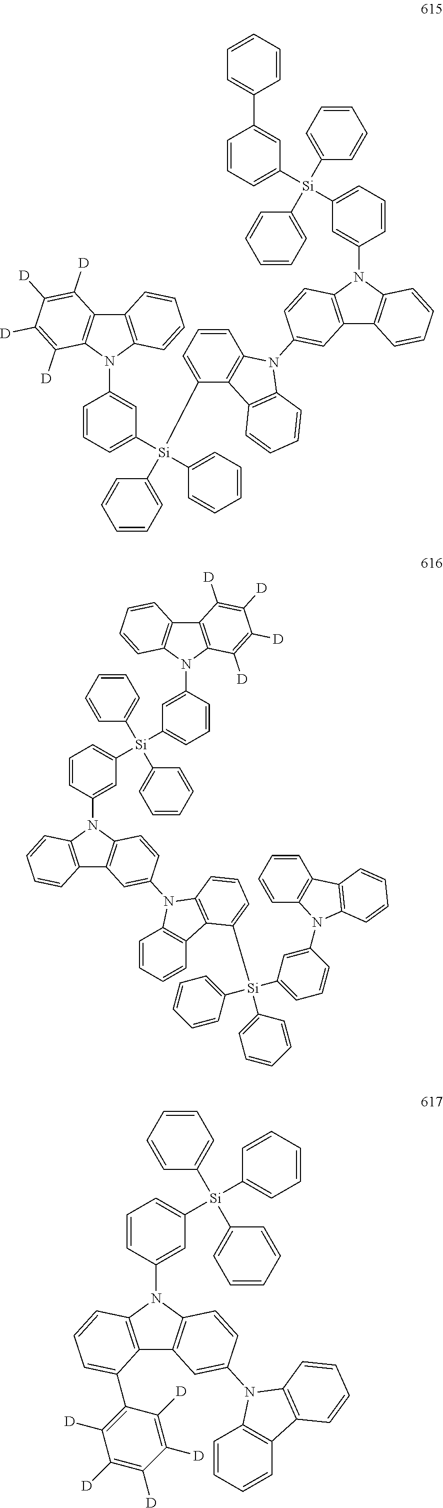 [SMILES: [2H]c1c([2H])c([2H])c2c(c1[2H])c1ccccc1n2-c1cccc([Si](c2ccccc2)(c2ccccc2)c2cccc3c2c2ccccc2n3-c2ccc3c(c2)c2ccccc2n3-c2cccc([Si](c3ccccc3)(c3ccccc3)c3cccc(-c4ccccc4)c3)c2)c1 ; [2H]c1c([2H])c([2H])c2c(c1[2H])c1ccccc1n2-c1cccc([Si](c2ccccc2)(c2ccccc2)c2cccc(-n3c4ccccc4c4cc(-n5c6ccccc6c6c([Si](c7ccccc7)(c7ccccc7)c7cccc(-n8c9ccccc9c9ccccc98)c7)cccc65)ccc43)c2)c1 ; [2H]c1c([2H])c([2H])c(-c2cccc3c2c2cc(-n4c5ccccc5c5ccccc54)ccc2n3-c2cccc([Si](c3ccccc3)(c3ccccc3)c3ccccc3)c2)c([2H])c1[2H]] 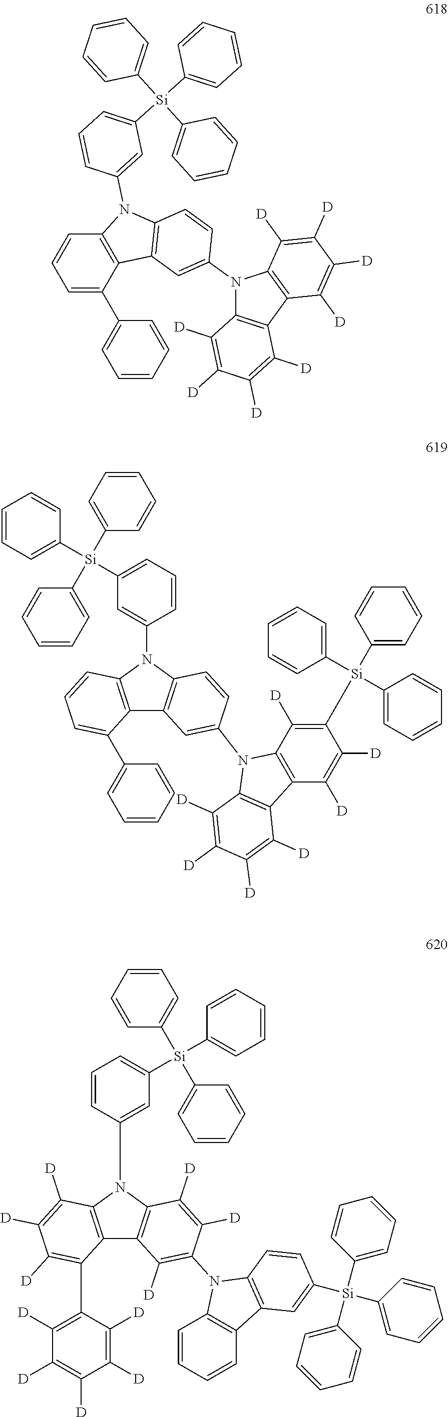 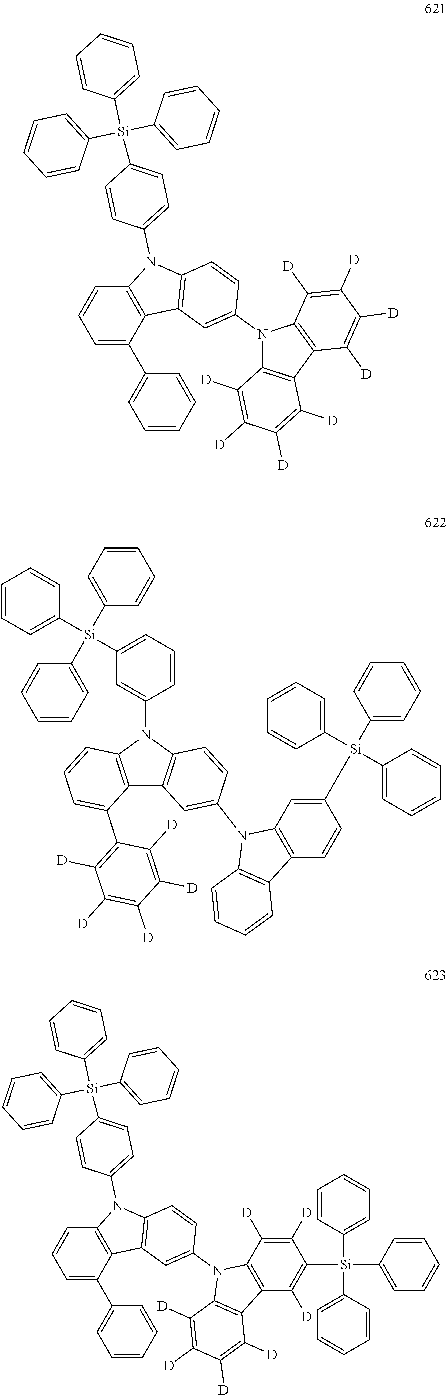 [SMILES: [2H]c1c([2H])c([2H])c2c(c1[2H])c1c([2H])c([2H])c([2H])c([2H])c1n2-c1ccc2c(c1)c1c(-c3ccccc3)cccc1n2-c1ccc([Si](c2ccccc2)(c2ccccc2)c2ccccc2)cc1 ; [2H]c1c([2H])c([2H])c(-c2cccc3c2c2cc(-n4c5ccccc5c5ccc([Si](c6ccccc6)(c6ccccc6)c6ccccc6)cc54)ccc2n3-c2cccc([Si](c3ccccc3)(c3ccccc3)c3ccccc3)c2)c([2H])c1[2H] ; [2H]c1c([2H])c([2H])c2c(c1[2H])c1c([2H])c([Si](c3ccccc3)(c3ccccc3)c3ccccc3)c([2H])c([2H])c1n2-c1ccc2c(c1)c1c(-c3ccccc3)cccc1n2-c1ccc([Si](c2ccccc2)(c2ccccc2)c2ccccc2)cc1] 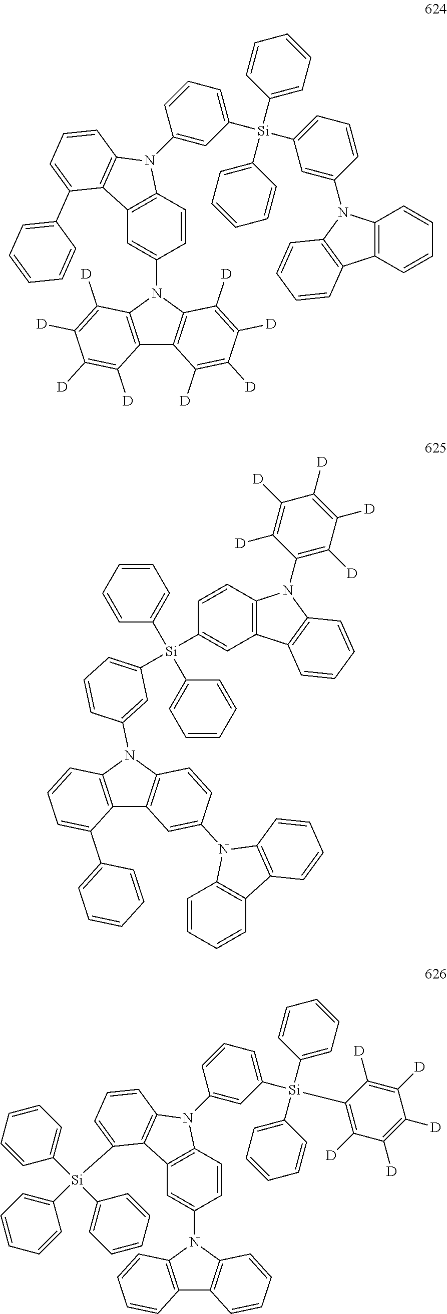 [SMILES: [2H]c1c([2H])c([2H])c2c(c1[2H])c1c([2H])c([2H])c([2H])c([2H])c1n2-c1ccc2c(c1)c1c(-c3ccccc3)cccc1n2-c1cccc([Si](c2ccccc2)(c2ccccc2)c2cccc(-n3c4ccccc4c4ccccc43)c2)c1 ; [2H]c1c([2H])c([2H])c(-n2c3ccccc3c3cc([Si](c4ccccc4)(c4ccccc4)c4cccc(-n5c6ccc(-n7c8ccccc8c8ccccc87)cc6c6c(-c7ccccc7)cccc65)c4)ccc32)c([2H])c1[2H] ; [2H]c1c([2H])c([2H])c([Si](c2ccccc2)(c2ccccc2)c2cccc(-n3c4ccc(-n5c6ccccc6c6ccccc65)cc4c4c([Si](c5ccccc5)(c5ccccc5)c5ccccc5)cccc43)c2)c([2H])c1[2H]] 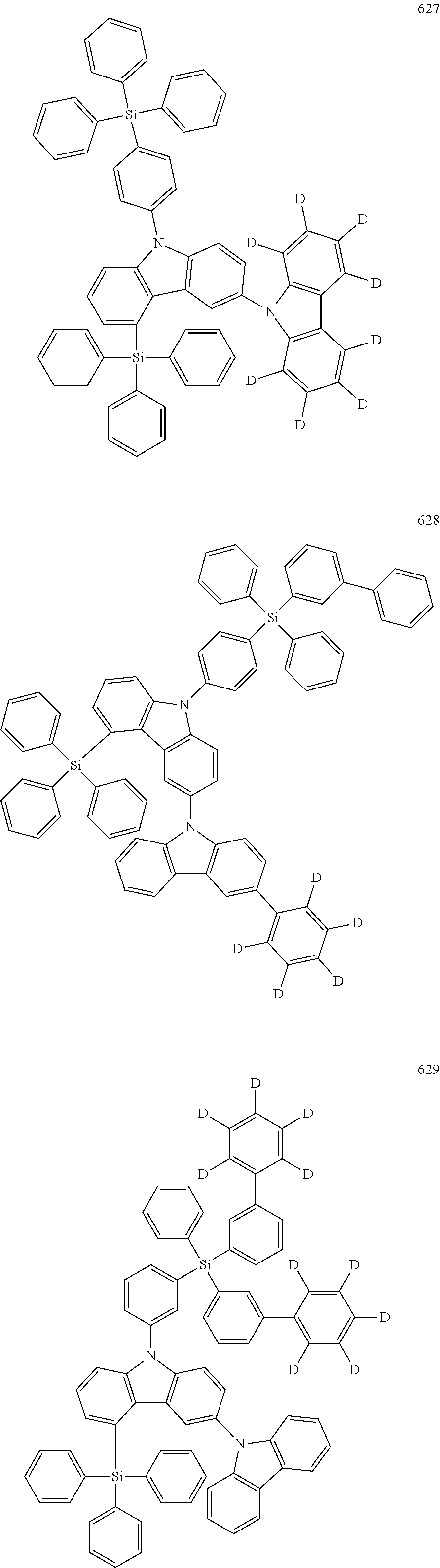 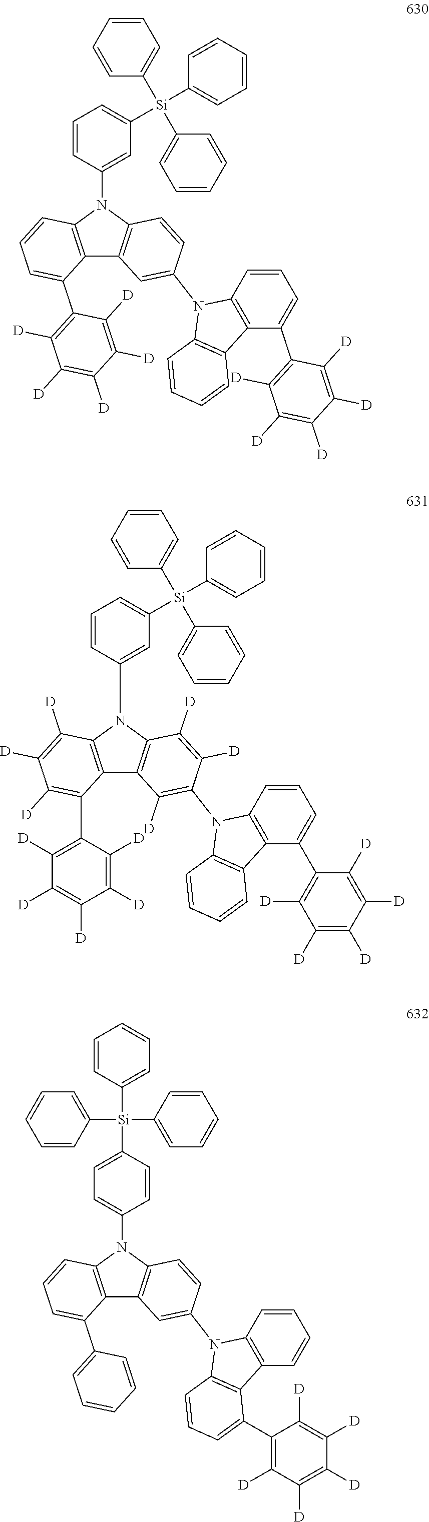 [SMILES: [2H]c1c([2H])c([2H])c(-c2cccc3c2c2ccccc2n3-c2ccc3c(c2)c2c(-c4c([2H])c([2H])c([2H])c([2H])c4[2H])cccc2n3-c2cccc([Si](c3ccccc3)(c3ccccc3)c3ccccc3)c2)c([2H])c1[2H] ; [2H]c1c([2H])c([2H])c(-c2cccc3c2c2ccccc2n3-c2c([2H])c([2H])c3c(c2[2H])c2c(-c4c([2H])c([2H])c([2H])c([2H])c4[2H])c([2H])c([2H])c([2H])c2n3-c2cccc([Si](c3ccccc3)(c3ccccc3)c3ccccc3)c2)c([2H])c1[2H] ; [2H]c1c([2H])c([2H])c(-c2cccc3c2c2ccccc2n3-c2ccc3c(c2)c2c(-c4ccccc4)cccc2n3-c2ccc([Si](c3ccccc3)(c3ccccc3)c3ccccc3)cc2)c([2H])c1[2H]] 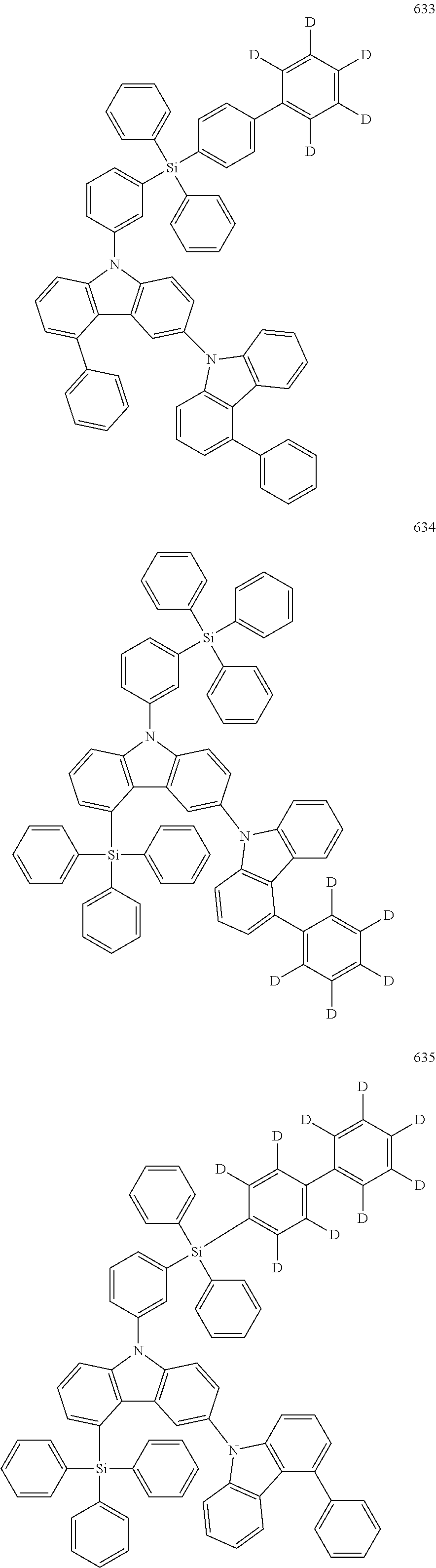 [SMILES: [2H]c1c([2H])c([2H])c(-c2ccc([Si](c3ccccc3)(c3ccccc3)c3cccc(-n4c5ccc(-n6c7ccccc7c7c(-c8ccccc8)cccc76)cc5c5c(-c6ccccc6)cccc54)c3)cc2)c([2H])c1[2H] ; [2H]c1c([2H])c([2H])c(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccccc3)c2)c([2H])c1[2H] ; [2H]c1c([2H])c([2H])c(-c2c([2H])c([2H])c([Si](c3ccccc3)(c3ccccc3)c3cccc(-n4c5ccc(-n6c7ccccc7c7c(-c8ccccc8)cccc76)cc5c5c([Si](c6ccccc6)(c6ccccc6)c6ccccc6)cccc54)c3)c([2H])c2[2H])c([2H])c1[2H]] 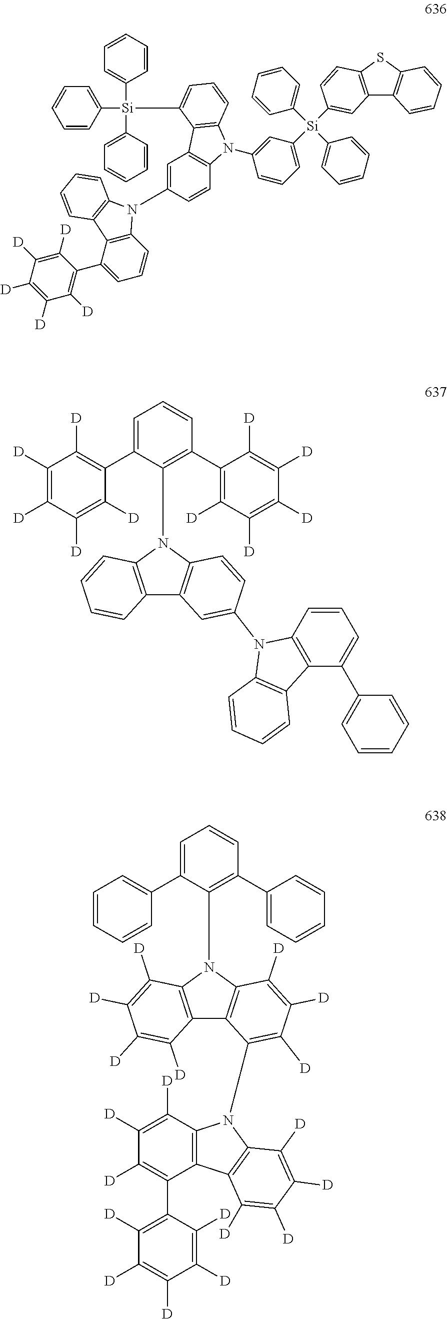 [SMILES: [2H]c1c([2H])c([2H])c(-c2cccc3c2c2ccccc2n3-c2ccc3c(c2)c2c([Si](c4ccccc4)(c4ccccc4)c4ccccc4)cccc2n3-c2cccc([Si](c3ccccc3)(c3ccccc3)c3ccc4sc5ccccc5c4c3)c2)c([2H])c1[2H] ; [2H]c1c([2H])c([2H])c(-c2cccc(-c3c([2H])c([2H])c([2H])c([2H])c3[2H])c2-n2c3ccccc3c3cc(-n4c5ccccc5c5c(-c6ccccc6)cccc54)ccc32)c([2H])c1[2H] ; [2H]c1c([2H])c([2H])c(-c2c([2H])c([2H])c([2H])c3c2c2c([2H])c([2H])c([2H])c([2H])c2n3-c2c([2H])c([2H])c([2H])c3c2c2c([2H])c([2H])c([2H])c([2H])c2n3-c2c(-c3ccccc3)cccc2-c2ccccc2)c([2H])c1[2H]] 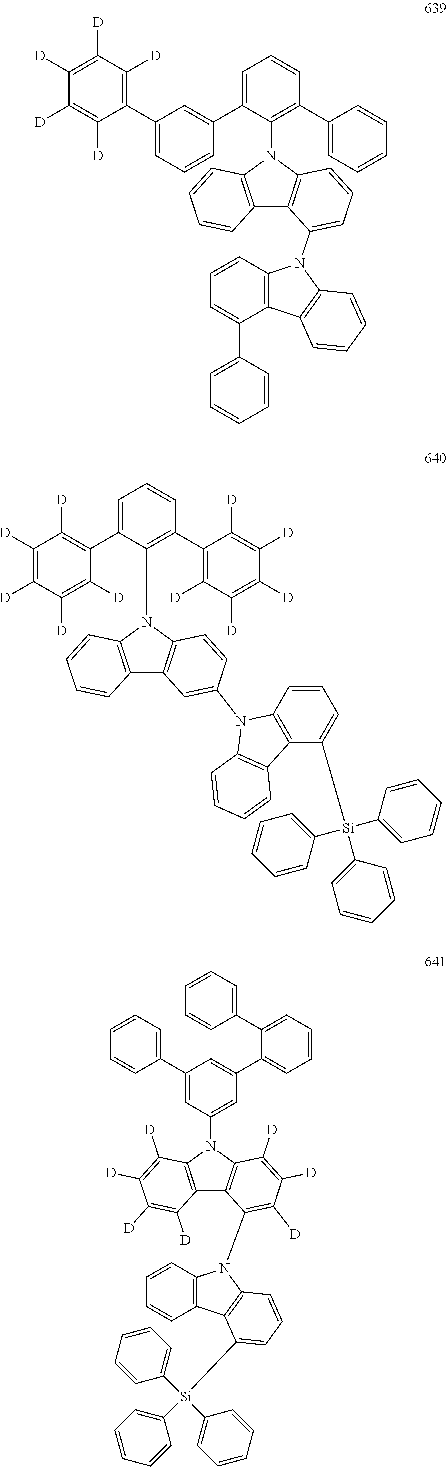 [SMILES: [2H]c1c([2H])c([2H])c(-c2cccc(-c3cccc(-c4ccccc4)c3-n3c4ccccc4c4c(-n5c6ccccc6c6c(-c7ccccc7)cccc65)cccc43)c2)c([2H])c1[2H] ; [2H]c1c([2H])c([2H])c(-c2cccc(-c3c([2H])c([2H])c([2H])c([2H])c3[2H])c2-n2c3ccccc3c3cc(-n4c5ccccc5c5c([Si](c6ccccc6)(c6ccccc6)c6ccccc6)cccc54)ccc32)c([2H])c1[2H] ; [2H]c1c([2H])c([2H])c2c(c1[2H])c1c(-n3c4ccccc4c4c([Si](c5ccccc5)(c5ccccc5)c5ccccc5)cccc43)c([2H])c([2H])c([2H])c1n2-c1cc(-c2ccccc2)cc(-c2ccccc2-c2ccccc2)c1] 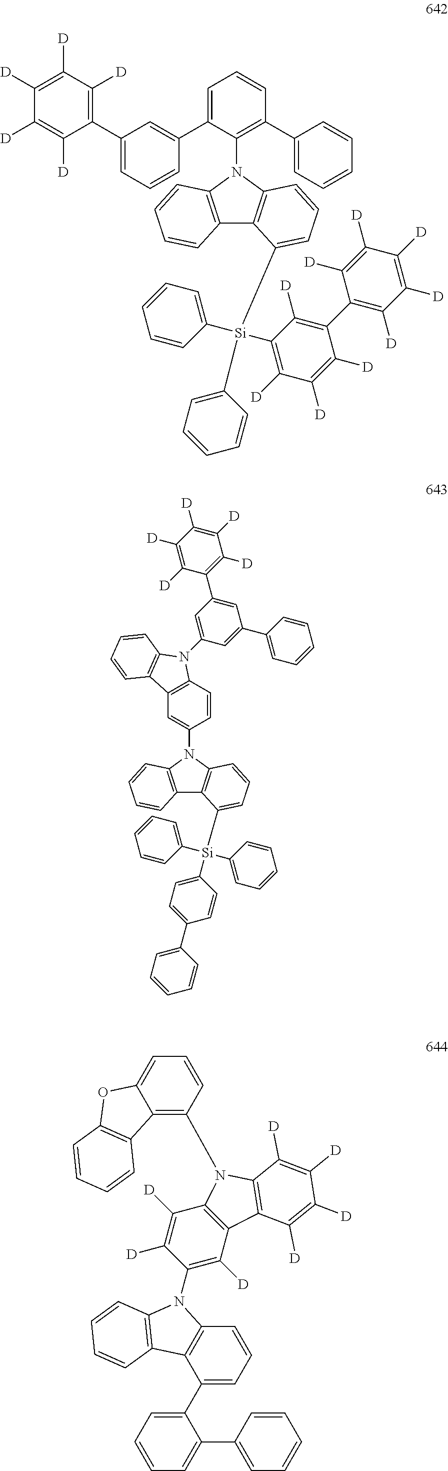 [SMILES: [2H]c1c([2H])c([2H])c(-c2cccc(-c3cccc(-c4ccccc4)c3-n3c4ccccc4c4c([Si](c5ccccc5)(c5ccccc5)c5c([2H])c([2H])c([2H])c(-c6c([2H])c([2H])c([2H])c([2H])c6[2H])c5[2H])cccc43)c2)c([2H])c1[2H] ; [2H]c1c([2H])c([2H])c(-c2cc(-c3ccccc3)cc(-n3c4ccccc4c4cc(-n5c6ccccc6c6c([Si](c7ccccc7)(c7ccccc7)c7ccc(-c8ccccc8)cc7)cccc65)ccc43)c2)c([2H])c1[2H] ; [2H]c1c([2H])c([2H])c2c(c1[2H])c1c([2H])c(-n3c4ccccc4c4c(-c5ccccc5-c5ccccc5)cccc43)c([2H])c([2H])c1n2-c1cccc2oc3ccccc3c12] 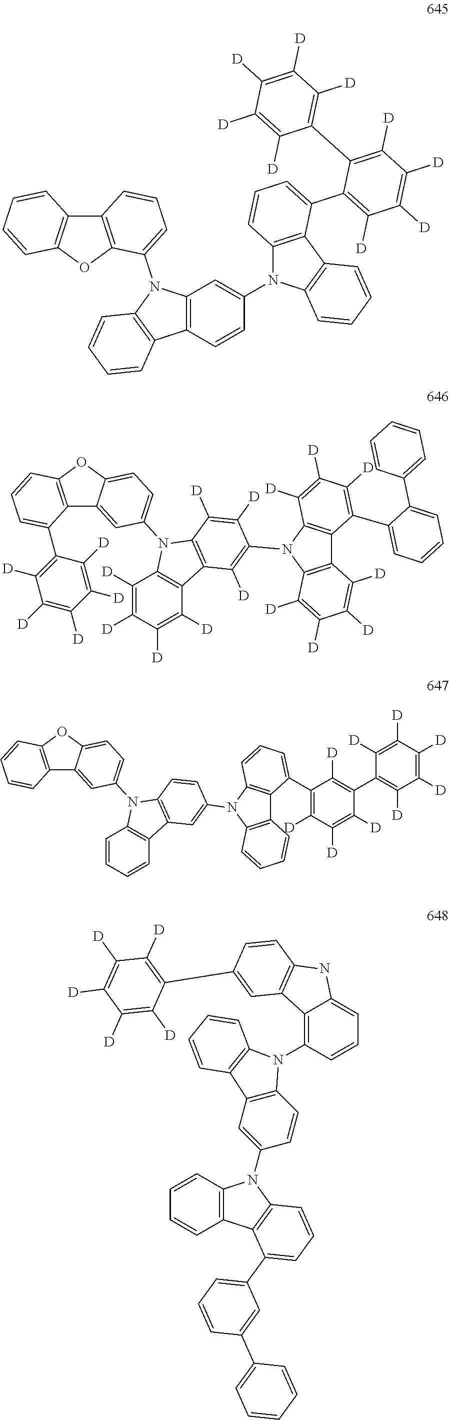 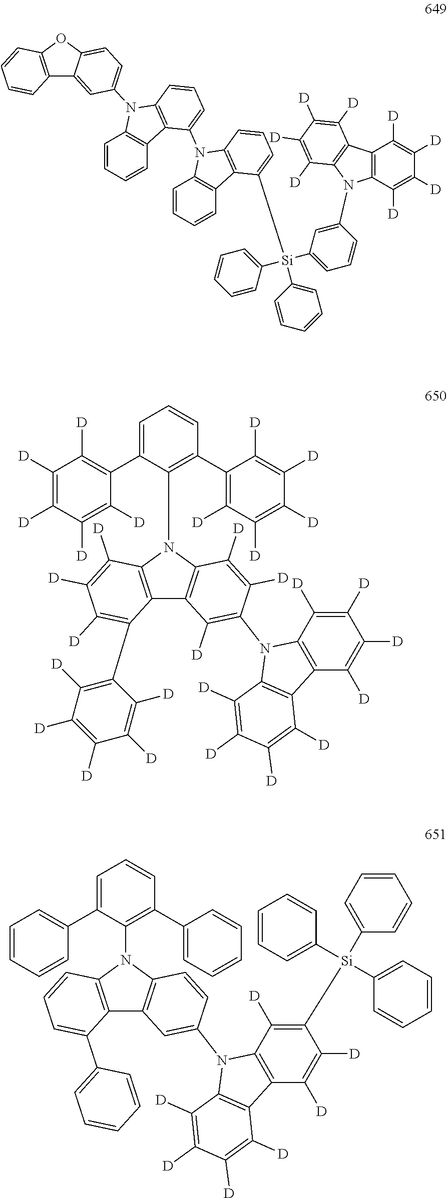 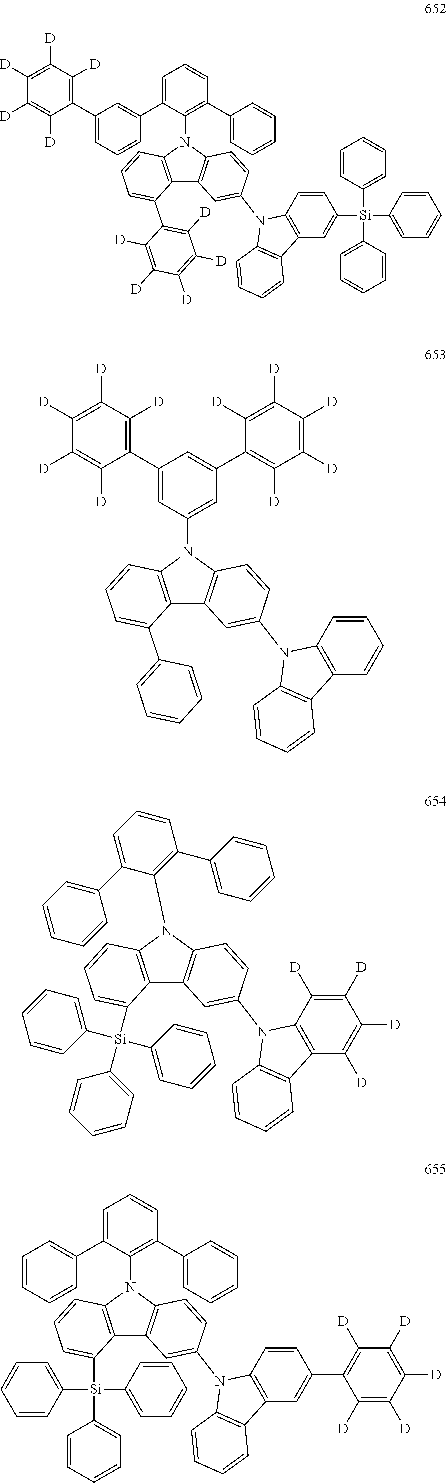 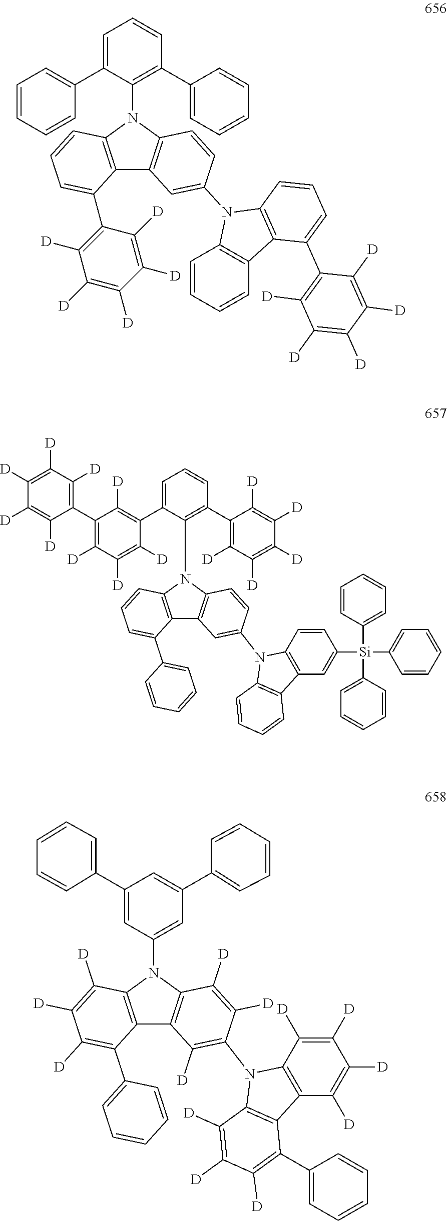 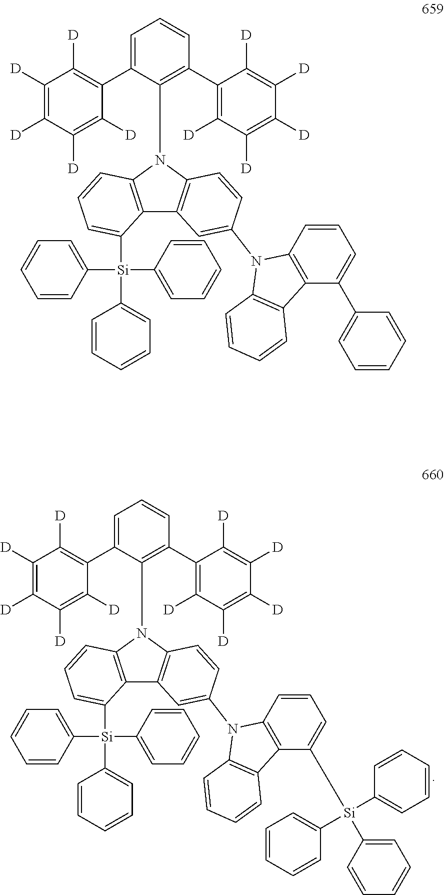 [SMILES: [2H]c1c([2H])c([2H])c(-c2cccc(-c3c([2H])c([2H])c([2H])c([2H])c3[2H])c2-n2c3ccc(-n4c5ccccc5c5c(-c6ccccc6)cccc54)cc3c3c([Si](c4ccccc4)(c4ccccc4)c4ccccc4)cccc32)c([2H])c1[2H] ; [2H]c1c([2H])c([2H])c(-c2cccc(-c3c([2H])c([2H])c([2H])c([2H])c3[2H])c2-n2c3ccc(-n4c5ccccc5c5c([Si](c6ccccc6)(c6ccccc6)c6ccccc6)cccc54)cc3c3c([Si](c4ccccc4)(c4ccccc4)c4ccccc4)cccc32)c([2H])c1[2H]]
